(19) United States
(12) Patent Application Publication
(10) Pub. No.: US 2025/0031568 A1
(43) Pub. Date: Jan. 23, 2025

(54) ORGANIC COMPOUND AND ORGANIC ELECTROLUMINESCENT DEVICE INCLUDING THE SAME

(71) Applicant: MATERIAL SCIENCE CO.,LTD., Seoul (KR)

(72) Inventors: Hani JEON, Seoul (KR); Tae Wan LEE, Seoul (KR); Jie SONG, Seoul (KR); Sang Hoon HONG, Seoul (KR)

(21) Appl. No.: 18/826,802

(22) Filed: Sep. 6, 2024

Related U.S. Application Data
(63) Continuation of application No. PCT/KR2023/002369, filed on Feb. 20, 2023.

(30) Foreign Application Priority Data
Mar. 10, 2022 (KR) 10-2022-0029756
Feb. 16, 2023 (KR) 10-2023-0020712

(51) Int. Cl. H10K 85/60; C07D 405/10; C07D 405/14; C07D 409/10; C07D 409/14; H10K 50/15

(52) U.S. Cl. CPC H10K 85/636; C07D 405/10; C07D 405/14; C07D 409/10; C07D 409/14; H10K 85/633; H10K 50/15; H10K 85/615; H10K 85/626; H10K 85/6572; H10K 85/6574; H10K 85/6576

(57) ABSTRACT

The present invention relates to a novel organic compound and an organic electroluminescent device comprising the same. More specifically, the present invention provides an organic electroluminescent device which has a low driving voltage and is significantly improved in terms of device efficiency characteristics and lifetime.

### CROSS-REFERENCE TO RELATED APPLICATION

The present application is a Continuation of PCT application number PCT/KR2023/002369, filed on Feb. 20, 2023, which is based upon and claims the benefit of priorities to Korean Patent Application Nos. 10-2023-0020712, filed on Feb. 16, 2023, and 10-2022-0029756 filed on Mar. 10, 2022, in the Korean Intellectual Property Office. All of the aforementioned applications are hereby incorporated by reference in their entireties.

### TECHNICAL FIELD

The present invention relates to an organic compound and an organic electroluminescent device including the same.

### BACKGROUND

Since organic electroluminescent devices have a simpler structure than other flat panel display devices such as current liquid crystal displays (LCDs), plasma display panels (PDPs), and field emission displays (FEDs), various advantages in a manufacturing process, high brightness and excellent viewing angle characteristics, and a fast response speed and a low driving voltage, the organic electroluminescent devices are being actively developed to be used as flat displays, such as wall-mounted TVs, or light sources, such as backlight of displays, lighting, and billboards.

In an organic electroluminescent device, generally, when a voltage is applied, holes injected from a positive electrode and electrons injected from a negative electrode are recombined to form an exciton, which is an electron-hole pair, and the energy of the exciton is transmitted to a light emitting material and converted into light.

To increase the efficiency and stability of the organic electroluminescent device, research for an organic material for a multi-layered organic electroluminescent device has been actively conducted since the low-voltage organic electroluminescent device in which the organic thin film is formed between two opposite electrodes was reported to by C. W. Tang, et al. of Eastman Kodak (C. W. Tang and S.A. Vanslyke, Applied Physics Letters, Volume 51, Pages 913, 1987).

Generally, the organic electroluminescent device has a structure which includes a negative electrode (electron injection electrode), a positive electrode (hole injection electrode), and one or more organic layers between the two electrodes. In this case, in the organic electroluminescent device, a hole injection layer (HIL), a hole transport layer (HTL), a light emitting layer (EML), an electron transport layer (ETL), or an electron injection layer (EIL) may be sequentially stacked from the positive electrode, and to increase the efficiency of the light emitting layer, an electron blocking layer (EBL) or a hole blocking layer (HBL) may be added in front of or behind the light emitting layer.

The reason why the organic electroluminescent device is manufactured in a multi-layer thin film structure is to stabilize an interface between the electrode and the organic material and increase luminous efficiency.

In particular, in the case of organic compounds used as materials for multi-layer thin films, since there is a large difference in movement speeds of holes and electrons according to their characteristics, holes and electrons may be effectively transported to the light emitting layer only when hole transport layers and electron transport layers containing appropriate compounds are used, and thus densities of the holes and the electrons may be balanced to excellently increase luminous efficiency.

Therefore, since the characteristics of the organic compound components contained in each layer of the organic thin film layer not only greatly affects a driving voltage, luminous efficiency, luminance, and lifetime of the device, but also affect the efficiency or lifetime of a finally produced display, it is important to use specific organic materials appropriate for the multi-layer structure in the organic electroluminescent devices. Therefore, research for the components included in each layer of the organic thin film layer is actively being conducted.

### DOCUMENTS OF RELATED ART

### Patent Documents

(Patent Document 1) U.S. Pat. No. 8,207,526 B2
(Patent Document 2) KR10-2019-0128179 A

The present invention is directed to providing an organic electroluminescent device which includes a novel organic compound and has a low driving voltage and excellent device efficiency characteristics and lifetime characteristics.

### SUMMARY

To achieve the object, the present invention may relate to a compound represented by Chemical Formula 1 below.

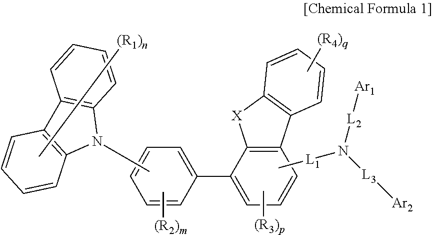

wherein,
n is an integer from 0 to 8,
m and q are the same as or different from each other and are each independently an integer from 0 to 4,
p is an integer from 0 to 2,
X is O or S,
L₁ to L₃ are the same as or different from each other and are each independently selected from the group consisting of a single bond, a substituted or unsubstituted arylene group having 6 to 30 carbon atoms, a substituted or unsubstituted heteroarylene group having 1 to 30 carbon atoms, a substituted or unsubstituted alkylene group having 1 to 10 carbon atoms, a substituted or unsubstituted cycloalkylene group having 3 to 10 carbon atoms, a substituted or unsubstituted alkenylene group having 2 to 10 carbon atoms, a substituted or unsubstituted cycloalkenylene group having 3 to 10 carbon atoms, a substituted or unsubstituted heteroalkylene group having 1 to 10 carbon atoms, a substituted or unsubstituted heterocycloalkylene group having 2 to 10 carbon atoms, a substituted or unsubstituted heteroalkenylene group having 2 to 10 carbon atoms, and a substituted or unsubstituted heterocycloalkenylene group having 2 to 10 carbon atoms,

Ar₁ and Ar₂ are selected from the group consisting of a substituted or unsubstituted aryl group having 6 to 30 carbon atoms, a substituted or unsubstituted heteroaryl group having 1 to 30 carbon atoms, a substituted or unsubstituted alkyl group having 1 to 20 carbon atoms, a substituted or unsubstituted cycloalkyl group having 3 to 20 carbon atoms, a substituted or unsubstituted heteroalkyl group having 1 to 20 carbon atoms, a substituted or unsubstituted heterocycloalkyl group having 2 to 20 carbon atoms, a substituted or unsubstituted aralkyl group having 6 to 30 carbon atoms, a substituted or unsubstituted alkenyl group having 2 to 20 carbon atoms, a substituted or unsubstituted cycloalkenyl group having 3 to 20 carbon atoms, and a substituted or unsubstituted heteroalkenyl groups having 2 to 20 carbon atoms,
R₁ to R₄ are the same as or different from each other, are each independently selected from the group consisting of hydrogen, deuterium, a cyano group, a nitro group, a halogen group, a hydroxy group, a substituted or unsubstituted alkylthio group having 1 to 4 carbon atoms, a substituted or unsubstituted alkyl group having 1 to 30 carbon atoms, a substituted or unsubstituted cycloalkyl group having 3 to 20 carbon atoms, a substituted or unsubstituted alkenyl group having 2 to 30 carbon atoms, a substituted or unsubstituted alkynyl group having 2 to 24 carbon atoms, a substituted or unsubstituted aralkyl group having 7 to 30 carbon atoms, a substituted or unsubstituted aryl group having 6 to 30 carbon atoms, a substituted or unsubstituted heteroaryl group having 1 to 60 carbon atoms, a substituted or unsubstituted heteroarylalkyl group having 2 to 30 carbon atoms, a substituted or unsubstituted alkoxy group having 1 to 30 carbon atoms, a substituted or unsubstituted alkylamino group having 1 to 30 carbon atoms, a substituted or unsubstituted arylamino group having 6 to 30 carbon atoms, a substituted or unsubstituted aralkylamino group having 7 to 30 carbon atoms, a substituted or unsubstituted heteroarylamino group having 1 to 24 carbon atoms, a substituted or unsubstituted alkylsilyl group having 1 to 30 carbon atoms, a substituted or unsubstituted arylsilyl group having 6 to 30 carbon atoms, and a substituted or unsubstituted aryloxy group having 6 to 30 carbon atoms.

In addition, the present invention relates to an organic electroluminescent device including a first electrode, a second electrode facing the first electrode, and one or more organic layer interposed between the first electrode and the second electrode, wherein the one or more organic layers include a compound represented by Chemical Formula 1.

In the present invention, “hydrogen” is hydrogen, protium, deuterium, or tritium, unless otherwise specified.

In the present invention, “halogen group” indicates fluorine, chlorine, bromine, or iodine.

In the present invention, “alkyl” indicates a monovalent substituent derived from a linear or branched saturated hydrocarbon having 1 to 40 carbon atoms. Examples thereof include methyl, ethyl, propyl, isobutyl, sec-butyl, pentyl, iso-amyl, hexyl, and the like, but are not limited thereto.

In the present invention, “alkenyl” indicates a monovalent substituent derived from a linear or branched unsaturated hydrocarbon having one or more carbon-carbon double bonds and 2 to 40 carbon atoms. Examples thereof include vinyl, allyl, isopropenyl, 2-butenyl, and the like, but are not limited thereto.

In the present invention, “alkynyl” indicates a monovalent substituent derived from a linear or branched unsaturated hydrocarbon having one or more carbon-carbon triple bond and 2 to 40 carbon atoms. Examples thereof include ethynyl, 2-propynyl, and the like, but are not limited thereto.

In the present invention, “alkylthio” indicates the above-described alkyl group bonded through a sulfur linkage (—S—).

In the present invention, “aryl” indicates a monovalent substituent derived from an aromatic hydrocarbon having 6 to 60 carbon atoms, which is a single ring or a combination of two or more rings. In addition, forms in which two or more rings are simply pendant or condensed, specifically, a naphthyl group, an anthracenyl group, a phenanthryl group, a triphenyl group, a pyrenyl group, a phenalenyl group, a perylenyl group, a chrysenyl group, a fluorenyl group, and the like may also be included, but the present invention is not limited thereto. The fluorenyl group may be substituted, and adjacent groups may be bonded to form a ring.

In the present invention, “heteroaryl” indicates a monovalent substituent derived from a monoheterocyclic or polyheterocyclic aromatic hydrocarbon having 6 to 30 carbon atoms. In this case, one or more carbons, preferably, 1 to 3 carbons of the ring are substituted with a heteroatom such as N, O, S or Se. In addition, a form in which two or more rings are simply pendant or condensed may be included, and a condensed form with an aryl group may also be included. Examples of such heteroaryls may include 6-membered monocyclic rings such as pyridyl, pyrazinyl, pyrimidinyl, pyridazinyl, and triazinyl, polycyclic rings such as phenoxathiinyl, indolizinyl, indolyl, purinyl, quinolyl, benzothiazole, and carbazolyl, 2-furanyl, N-imidazolyl, 2-isoxazolyl, 2-pyridinyl, 2-pyrimidinyl, and the like, but are not limited thereto.

In the present invention, “aryloxy” indicates a monovalent substituent represented by RO—, in which R indicates aryl having 6 to 60 carbon atoms. Examples of such aryloxy may include phenyloxy, naphthyloxy, diphenyloxy, and the like, but are not limited thereto.

In the present invention, “alkyloxy” indicates a monovalent substituent represented by R′O—, in which R′ indicates alkyl having 1 to 40 carbon atoms and may include a linear, branched, or cyclic structure. Examples of alkyloxy may include methoxy, ethoxy, n-propoxy, 1-propoxy, t-butoxy, n-butoxy, and pentoxy, but are not limited thereto.

In the present invention, “alkoxy” may be linear, branched chain, or ring chain. The number of carbon atoms of alkoxy is not especially limited, but is preferably 1 to 20. Specifically, the alkoxy may be methoxy, ethoxy, n-propoxy, isopropoxy, i-propyloxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy, and the like, but is not limited thereto.

In the present invention, “aralkyl” indicates an aryl-alkyl group in which aryl and alkyl are described above. Preferred aralkyl includes lower alkyl groups. Non-limiting examples of suitable aralkyl groups include benzyl, 2-phenethyl, and naphthalenylmethyl. Bonding to the parent moiety is made via the alkyl.

In the present invention, “arylamino group” indicates an amine substituted with an aryl group having 6 to 30 carbon atoms.

In the present invention, “alkylamino group” indicates an amine substituted with an alkyl group having 1 to 30 carbon atoms.

In the present invention, “aralkylamino group” indicates an amine substituted with an aryl-alkyl group having 6 to 30 carbon atoms.

In the present invention, “heteroarylamino group” indicates an amine group substituted with an aryl group and heterocyclic group having 6 to 30 carbon atoms.

In the present invention, “heteroaralkyl group” indicates an aryl-alkyl group substituted with a heterocyclic group.

In the present invention, “cycloalkyl” indicates a monovalent substituent derived from a monocyclic or polycyclic non-aromatic hydrocarbon having 3 to 40 carbon atoms. Examples of such cycloalkyl may include cyclopropyl, cyclobutyl, cyclopentyl, cyclohexyl, norbornyl, adamantine, and the like, but are not limited thereto.

In the present invention, “heterocycloalkyl” indicates a monovalent substituent derived from a non-aromatic hydrocarbon having 3 to 40 carbon atoms, and one or more carbons, preferably, 1 to 3 carbons of the ring are substituted with a hetero atom such as N, O, S or Se. Examples of such heterocycloalkyl may include morpholine, piperazine, and the like, but are not limited thereto.

In the present invention, “alkylsilyl” indicates silyl substituted with alkyl having 1 to 40 carbon atoms, and “arylsilyl” indicates silyl substituted with aryl having 6 to 60 carbon atoms.

In the present invention, “condensed ring” indicates a condensed aliphatic ring, a condensed aromatic ring, a condensed heteroaliphatic ring, a condensed heteroaromatic ring, or a combination thereof.

In the present invention, “forming a ring by being bonded with adjacent groups” indicates forming a substituted or unsubstituted aliphatic hydrocarbon ring; a substituted or unsubstituted aromatic hydrocarbon ring; a substituted or unsubstituted aliphatic heterocycle; a substituted or unsubstituted aromatic heterocycle; or a condensed ring thereof by being bonded with adjacent groups.

In the present invention, examples of “aromatic hydrocarbon rings” may include a phenyl group, a naphthyl group, an anthracenyl group and the like, but are not limited thereto.

In the present invention, “aliphatic heterocycle” indicates an aliphatic ring including one or more heteroatoms.

In the present invention, “aromatic heterocycle” indicates an aromatic ring including one or more heteroatoms.

In the present invention, “substitution” indicates substituting a hydrogen atom bonded to a carbon atom of a compound with another substituent, and a location to be substituted is not limited as long as it is a location at which the hydrogen atom is substituted, that is, a location at which the substituent may be substituted, and when two or more substituents are substituted, the two or more substituents may be the same as or different from each other. The substituent may be substituted with one or more substituents selected from the group consisting of deuterium, a cyano group, a nitro group, a halogen group, a hydroxy group, an alkyl group having 1 to 30 carbon atoms, an alkenyl group having 2 to 30 carbon atoms, an alkynyl group having 2 to 24 carbon atoms, a heteroalkyl group having 2 to 30 carbon atoms, an aralkyl group having 6 to 30 carbon atoms, an aryl group having 5 to 30 carbon atoms, a heteroaryl group having 2 to 30 carbon atoms, a heteroarylalkyl group having 3 to 30 carbon atoms, an alkoxy group having 1 to 30 carbon atoms, an alkylamino group having 1 to 30 carbon atoms, an arylamino group having 6 to 30 carbon atoms, an aralkylamino group having 6 to 30 carbon atoms, a heteroarylamino group having 2 to 24 carbon atoms, a substituted or unsubstituted alkylsilyl group having 1 to 30 carbon atoms, a substituted or unsubstituted arylsilyl group having 6 to 30 carbon atoms, and a substituted or unsubstituted aryloxy group having 6 to 30 carbon atoms, but is not limited to the above examples.

### Advantageous Effects

The present invention can provide the novel organic compound and can have excellent interface characteristics with adjacent layers and excellent chemical stability when used as a material for an organic electroluminescent device.

In addition, the present invention can provide the organic electroluminescent device which includes the novel organic compound and has a low driving voltage and excellent device efficiency characteristics and lifetime characteristics.

### MODE FOR INVENTION

The present invention relates to a compound represented by Chemical Formula 1 below.

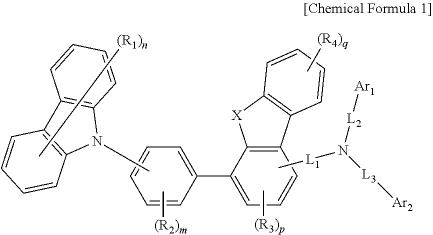

wherein,
n is an integer from 0 to 8,
m and q are the same as or different from each other and are each independently an integer from 0 to 4,
p is an integer from 0 to 2,
X is O or S,
L₁ to L₃ are the same as or different from each other and are each independently selected from the group consisting of a single bond, a substituted or unsubstituted arylene group having 6 to 30 carbon atoms, a substituted or unsubstituted heteroarylene group having 1 to 30 carbon atoms, a substituted or unsubstituted alkylene group having 1 to 10 carbon atoms, a substituted or unsubstituted cycloalkylene group having 3 to 10 carbon atoms, a substituted or unsubstituted alkenylene group having 2 to 10 carbon atoms, a substituted or unsubstituted cycloalkenylene group having 3 to 10 carbon atoms, a substituted or unsubstituted heteroalkylene group having 1 to 10 carbon atoms, a substituted or unsubstituted heterocycloalkylene group having 2 to 10 carbon atoms, a substituted or unsubstituted heteroalkenylene group having 2 to 10 carbon atoms, and a substituted or unsubstituted heterocycloalkenylene group having 2 to 10 carbon atoms,
Ar₁ and Ar₂ are the same as or different from each other and are each independently selected from the group consisting of a substituted or unsubstituted aryl group having 6 to 30 carbon atoms, a substituted or unsubstituted heteroaryl group having 1 to 30 carbon atoms, a substituted or unsubstituted alkyl group having 1 to 20 carbon atoms, a substituted or unsubstituted cycloalkyl group having 3 to 20 carbon atoms, a substituted or unsubstituted heteroalkyl group having 1 to 20 carbon atoms, a substituted or unsubstituted heterocycloalkyl group having 2 to 20 carbon atoms, a substituted or unsubstituted aralkyl group having 6 to 30 carbon atoms, a substituted or unsubstituted alkenyl group having 2 to 20 carbon atoms, a substituted or unsubstituted cycloalkenyl group having 3 to 20 carbon atoms, and a substituted or unsubstituted heteroalkenyl groups having 2 to 20 carbon atoms,
R₁ to R₄ are the same as or different from each other and are each independently selected from the group consisting of hydrogen, deuterium, a cyano group, a nitro group, a halogen group, a hydroxy group, a substituted or unsubstituted alkylthio group having 1 to 4 carbon atoms, a substituted or unsubstituted alkyl group having 1 to 30 carbon atoms, a substituted or unsubstituted cycloalkyl group having 3 to 20 carbon atoms, a substituted or unsubstituted alkenyl group having 2 to 30 carbon atoms, a substituted or unsubstituted alkynyl group having 2 to 24 carbon atoms, a substituted or unsubstituted aralkyl group having 7 to 30 carbon atoms, a substituted or unsubstituted aryl group having 6 to 30 carbon atoms, a substituted or unsubstituted heteroaryl group having 1 to 60 carbon atoms, a substituted or unsubstituted heteroarylalkyl group having 2 to 30 carbon atoms, a substituted or unsubstituted alkoxy group having 1 to 30 carbon atoms, a substituted or unsubstituted alkylamino group having 1 to 30 carbon atoms, a substituted or unsubstituted arylamino group having 6 to 30 carbon atoms, a substituted or unsubstituted aralkylamino group having 7 to 30 carbon atoms, a substituted or unsubstituted heteroarylamino group having 1 to 24 carbon atoms, a substituted or unsubstituted alkylsilyl group having 1 to 30 carbon atoms, a substituted or unsubstituted arylsilyl group having 6 to 30 carbon atoms, and a substituted or unsubstituted aryloxy group having 6 to 30 carbon atoms.

### DETAILED DESCRIPTION

Hereinafter, embodiments of the present invention will be described in detail so that those skilled in the art can easily carry out the present invention. However, the present invention may be implemented in various different forms and is not limited to the embodiments described herein.

A novel organic compound according to the present invention may have excellent interface characteristics with adjacent layers and excellent chemical stability, and in particular, have a HOMO energy level at which hole transport is easy and thus may be used as a material for a hole transport auxiliary layer of the organic electroluminescent device with excellent hole transport characteristics to a light emitting layer.

Specifically, a compound represented by Chemical Formula 1 below is as follows:

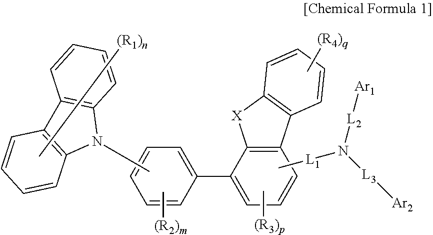

wherein,
n is an integer from 0 to 8,
m and q are the same as or different from each other and are each independently an integer from 0 to 4,
p is an integer from 0 to 2,
X is O or S,
L₁ to L₃ are the same as or different from each other and are each independently selected from the group consisting of a single bond, a substituted or unsubstituted arylene group having 6 to 30 carbon atoms, a substituted or unsubstituted heteroarylene group having 1 to 30 carbon atoms, a substituted or unsubstituted alkylene group having 1 to 10 carbon atoms, a substituted or unsubstituted cycloalkylene group having 3 to 10 carbon atoms, a substituted or unsubstituted alkenylene group having 2 to 10 carbon atoms, a substituted or unsubstituted cycloalkenylene group having 3 to 10 carbon atoms, a substituted or unsubstituted heteroalkylene group having 1 to 10 carbon atoms, a substituted or unsubstituted heterocycloalkylene group having 2 to 10 carbon atoms, a substituted or unsubstituted heteroalkenylene group having 2 to 10 carbon atoms, and a substituted or unsubstituted heterocycloalkenylene group having 2 to 10 carbon atoms,
Ar₁ and Ar₂ are the same as or different from each other and are each independently selected from the group consisting of a substituted or unsubstituted aryl group having 6 to 30 carbon atoms, a substituted or unsubstituted heteroaryl group having 1 to 30 carbon atoms, a substituted or unsubstituted alkyl group having 1 to 20 carbon atoms, a substituted or unsubstituted cycloalkyl group having 3 to 20 carbon atoms, a substituted or unsubstituted heteroalkyl group having 1 to 20 carbon atoms, a substituted or unsubstituted heterocycloalkyl group having 2 to 20 carbon atoms, a substituted or unsubstituted aralkyl group having 6 to 30 carbon atoms, a substituted or unsubstituted alkenyl group having 2 to 20 carbon atoms, a substituted or unsubstituted cycloalkenyl group having 3 to 20 carbon atoms, and a substituted or unsubstituted heteroalkenyl groups having 2 to 20 carbon atoms,
R₁ to R₄ are the same as or different from each other and are each independently selected from the group consisting of hydrogen, deuterium, a cyano group, a nitro group, a halogen group, a hydroxy group, a substituted or unsubstituted alkylthio group having 1 to 4 carbon atoms, a substituted or unsubstituted alkyl group having 1 to 30 carbon atoms, a substituted or unsubstituted cycloalkyl group having 3 to 20 carbon atoms, a substituted or unsubstituted alkenyl group having 2 to 30 carbon atoms, a substituted or unsubstituted alkynyl group having 2 to 24 carbon atoms, a substituted or unsubstituted aralkyl group having 7 to 30 carbon atoms, a substituted or unsubstituted aryl group having 6 to 30 carbon atoms, a substituted or unsubstituted heteroaryl group having 1 to 60 carbon atoms, a substituted or unsubstituted heteroarylalkyl group having 2 to 30 carbon atoms, a substituted or unsubstituted alkoxy group having 1 to 30 carbon atoms, a substituted or unsubstituted alkylamino group having 1 to 30 carbon atoms, a substituted or unsubstituted arylamino group having 6 to 30 carbon atoms, a substituted or unsubstituted aralkylamino group having 7 to 30 carbon atoms, a substituted or unsubstituted heteroarylamino group having 1 to 24 carbon atoms, a substituted or unsubstituted alkylsilyl group having 1 to 30 carbon atoms, a substituted or unsubstituted arylsilyl group having 6 to 30 carbon atoms, and a substituted or unsubstituted aryloxy group having 6 to 30 carbon atoms.

The compound represented by Chemical Formula 1 may be compounds represented by Chemical Formulas 2 to 4 below:

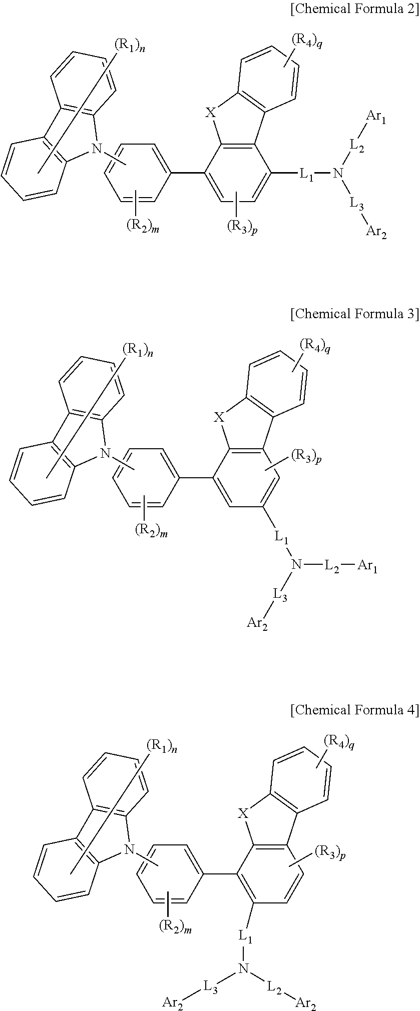

wherein,
n, m, p, q, X, L₁ to L₃, Ar₁, Ar₂, and R₁ to R₄ are same as defined in Chemical Formula 1.

The compound represented by Chemical Formula 2 may be compounds represented by Chemical Formulas 5 to 7 below:

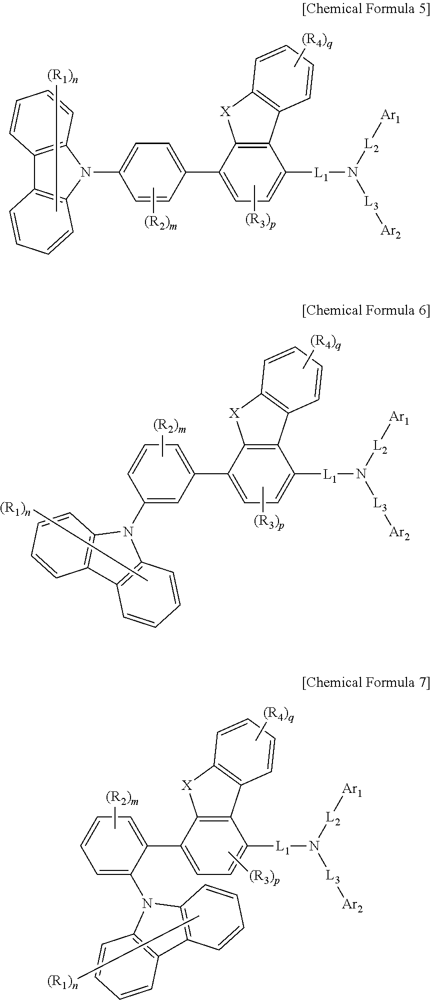

wherein,
n, m, p, q, X, L₁ to L₃, Ar₁, Ar₂, and R₁ to R₄ are same as defined in Chemical Formula 1.

The compound represented by Chemical Formula 3 may be compounds represented by Chemical Formulas 8 to 10 below:

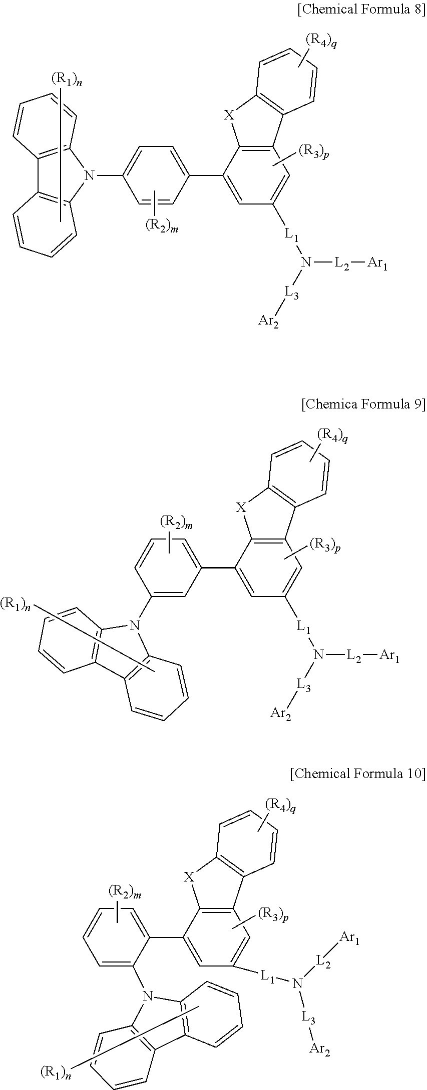

wherein,
n, m, p, q, X, L₁ to L₃, Ar₁, Ar₂, and R₁ to R₄ are same as defined in Chemical Formula 1.

The compound represented by Chemical Formula 4 may be compounds represented by Chemical Formulas 11 to 13 below:

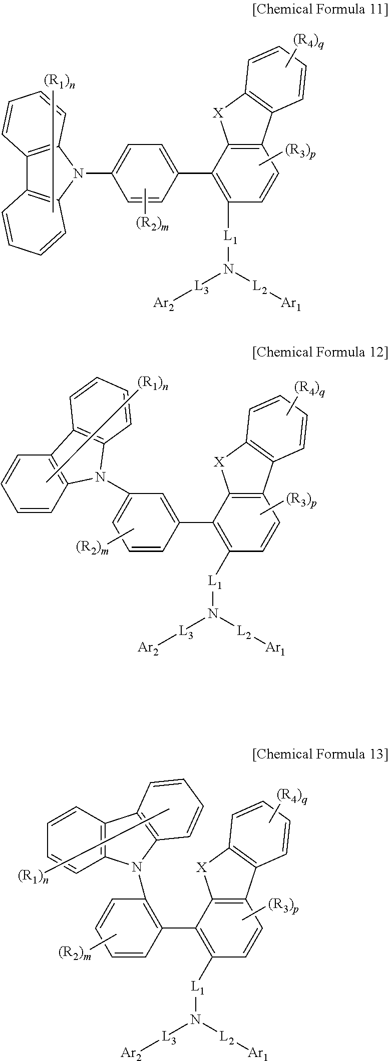

wherein,
n, m, p, q, X, L₁ to L₃, Ar₁, Ar₂, and R₁ to R₄ are same as defined in Chemical Formula 1.
L₁ to L₃ are the same as or different from each other and are each independently be selected from the group consisting of a single bond, a substituted or unsubstituted arylene group having 6 to 30 carbon atoms, and a substituted or unsubstituted heteroarylene group having 1 to 30 carbon atoms.
Ar₁ and Ar₂ are the same as or different from each other and are each independently may be selected from the group consisting of compounds represented by Chemical Formulas 14 to 17 below:

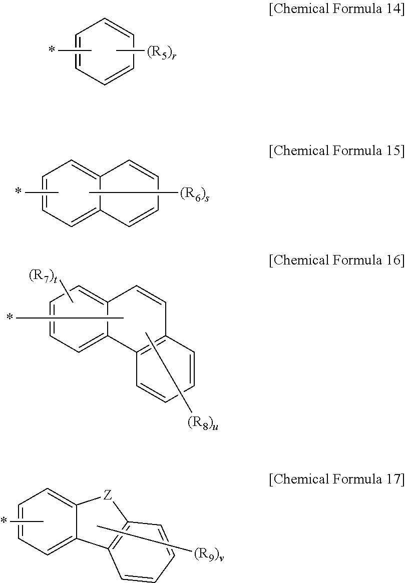

wherein,
* indicates a portion which is bonded,
r is an integer from 0 to 5,
s and v are the same as or different from each other and are each independently an integer from 0 to 7,
t is an integer from 0 to 4,
u is an integer from 0 to 6,
Z is C(R₁₀)(R₁₁), N(R₁₂), O or S,
R₅ to R₁₂ are the same as or different from each other and are each independently selected from the group consisting of hydrogen, deuterium, a cyano group, a nitro group, a halogen group, a hydroxy group, a substituted or unsubstituted alkylthio group having 1 to 4 carbon atoms, a substituted or unsubstituted alkyl group having 1 to 30 carbon atoms, a substituted or unsubstituted cycloalkyl group having 3 to 20 carbon atoms, a substituted or unsubstituted alkenyl group having 2 to 30 carbon atoms, a substituted or unsubstituted alkynyl group having 2 to 24 carbon atoms, a substituted or unsubstituted aralkyl group having 7 to 30 carbon atoms, a substituted or unsubstituted aryl group having 6 to 30 carbon atoms, a substituted or unsubstituted heteroaryl group having 1 to 60 carbon atoms, a substituted or unsubstituted heteroarylalkyl group having 2 to 30 carbon atoms, a substituted or unsubstituted alkoxy group having 1 to 30 carbon atoms, a substituted or unsubstituted alkylamino group having 1 to 30 carbon atoms, a substituted or unsubstituted arylamino group having 6 to 30 carbon atoms, a substituted or unsubstituted aralkylamino group having 7 to 30 carbon atoms, a substituted or unsubstituted heteroarylamino group having 1 to 24 carbon atoms, a substituted or unsubstituted alkylsilyl group having 1 to 30 carbon atoms, a substituted or unsubstituted arylsilyl group having 6 to 30 carbon atoms, and a substituted or unsubstituted aryloxy group having 6 to 30 carbon atoms.
Ar₁ and Ar₂ are the same as or different from each other and are each independently selected from the group consisting of a substituted or unsubstituted phenylene group, a substituted or unsubstituted biphenylene group, a substituted or unsubstituted terphenylene group, a substituted or unsubstituted naphthalenyl group, a substituted or unsubstituted phenanthrenyl group, a substituted or unsubstituted fluorene group, a substituted or unsubstituted spirofluorene group, a substituted or unsubstituted dibenzofuran group, a substituted or unsubstituted dibenzothiophene group, a substituted or unsubstituted furan group, and a substituted or unsubstituted carbazole group.

The compound represented by Chemical Formula 1 according to the present invention is selected from the group consisting of the following compounds, but is not limited thereto:

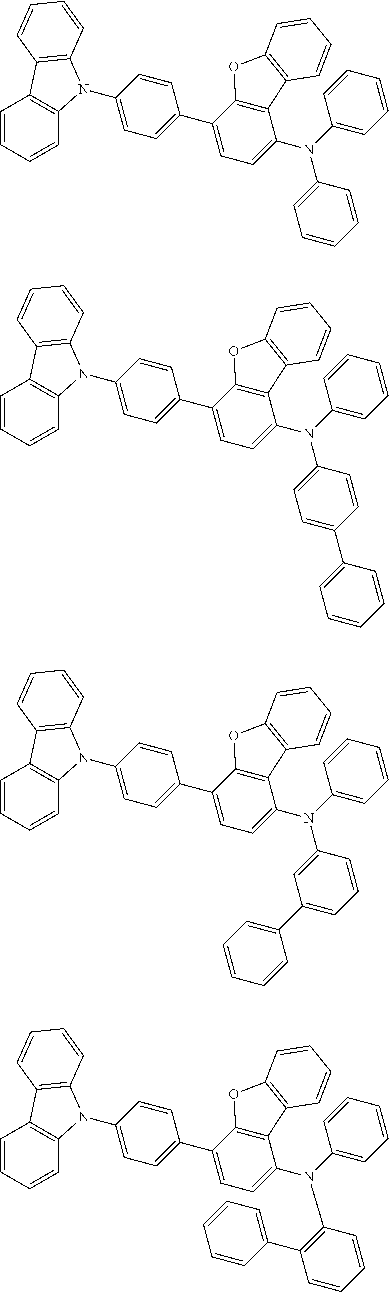 [SMILES: c1ccc(N(c2ccccc2)c2ccc(-c3ccc(-n4c5ccccc5c5ccccc54)cc3)c3oc4ccccc4c23)cc1 ; c1ccc(-c2ccc(N(c3ccccc3)c3ccc(-c4ccc(-n5c6ccccc6c6ccccc65)cc4)c4oc5ccccc5c34)cc2)cc1 ; c1ccc(-c2cccc(N(c3ccccc3)c3ccc(-c4ccc(-n5c6ccccc6c6ccccc65)cc4)c4oc5ccccc5c34)c2)cc1 ; c1ccc(-c2ccccc2N(c2ccccc2)c2ccc(-c3ccc(-n4c5ccccc5c5ccccc54)cc3)c3oc4ccccc4c23)cc1]

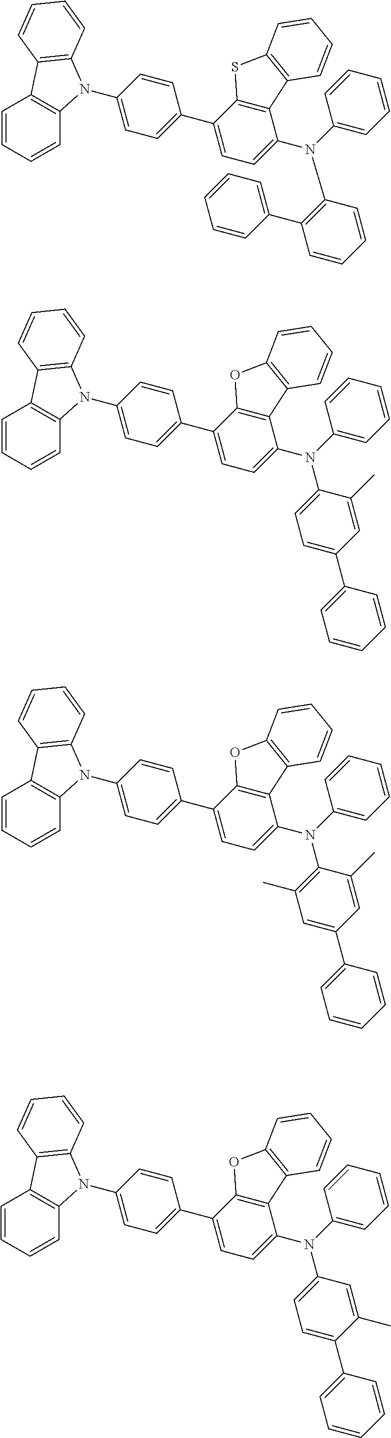 [SMILES: c1ccc(-c2ccccc2N(c2ccccc2)c2ccc(-c3ccc(-n4c5ccccc5c5ccccc54)cc3)c3sc4ccccc4c23)cc1 ; Cc1cc(-c2ccccc2)ccc1N(c1ccccc1)c1ccc(-c2ccc(-n3c4ccccc4c4ccccc43)cc2)c2oc3ccccc3c12 ; Cc1cc(-c2ccccc2)cc(C)c1N(c1ccccc1)c1ccc(-c2ccc(-n3c4ccccc4c4ccccc43)cc2)c2oc3ccccc3c12 ; Cc1cc(N(c2ccccc2)c2ccc(-c3ccc(-n4c5ccccc5c5ccccc54)cc3)c3oc4ccccc4c23)ccc1-c1ccccc1]

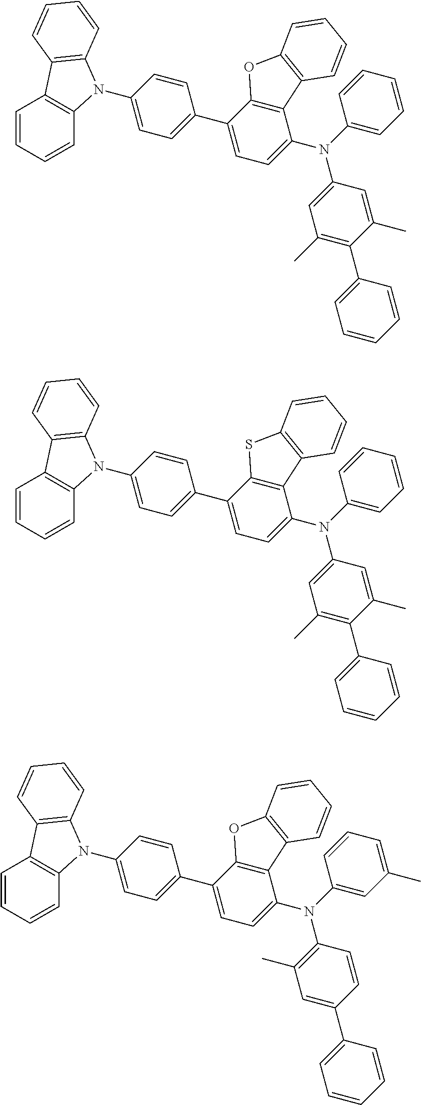 [SMILES: Cc1cc(N(c2ccccc2)c2ccc(-c3ccc(-n4c5ccccc5c5ccccc54)cc3)c3oc4ccccc4c23)cc(C)c1-c1ccccc1 ; Cc1cc(N(c2ccccc2)c2ccc(-c3ccc(-n4c5ccccc5c5ccccc54)cc3)c3sc4ccccc4c23)cc(C)c1-c1ccccc1 ; Cc1cccc(N(c2ccc(-c3ccccc3)cc2C)c2ccc(-c3ccc(-n4c5ccccc5c5ccccc54)cc3)c3oc4ccccc4c23)c1]

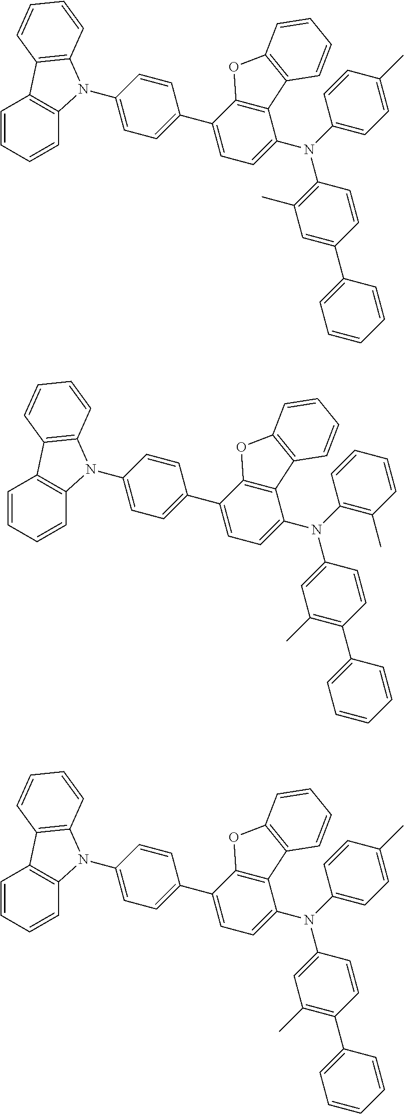 [SMILES: Cc1ccc(N(c2ccc(-c3ccccc3)cc2C)c2ccc(-c3ccc(-n4c5ccccc5c5ccccc54)cc3)c3oc4ccccc4c23)cc1 ; Cc1cc(N(c2ccccc2C)c2ccc(-c3ccc(-n4c5ccccc5c5ccccc54)cc3)c3oc4ccccc4c23)ccc1-c1ccccc1 ; Cc1ccc(N(c2ccc(-c3ccccc3)c(C)c2)c2ccc(-c3ccc(-n4c5ccccc5c5ccccc54)cc3)c3oc4ccccc4c23)cc1]

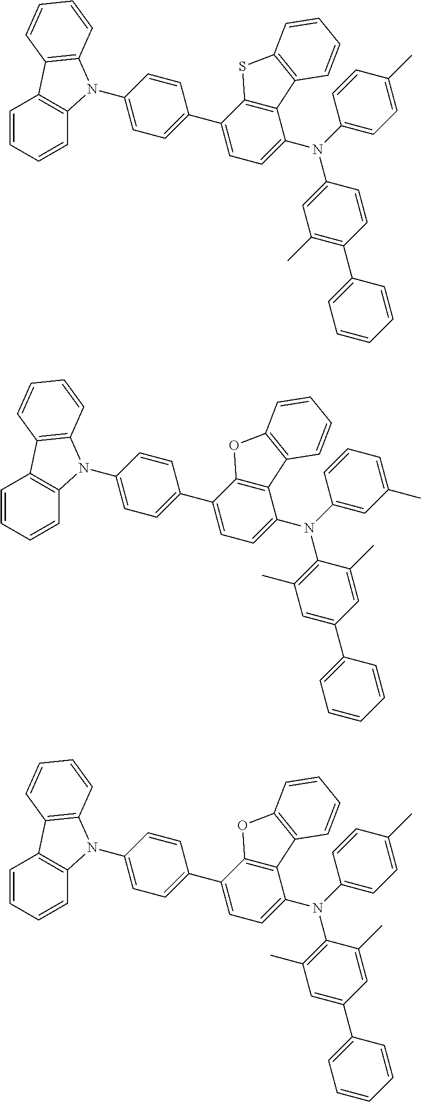 [SMILES: Cc1ccc(N(c2ccc(-c3ccccc3)c(C)c2)c2ccc(-c3ccc(-n4c5ccccc5c5ccccc54)cc3)c3sc4ccccc4c23)cc1 ; Cc1cccc(N(c2c(C)cc(-c3ccccc3)cc2C)c2ccc(-c3ccc(-n4c5ccccc5c5ccccc54)cc3)c3oc4ccccc4c23)c1 ; Cc1ccc(N(c2c(C)cc(-c3ccccc3)cc2C)c2ccc(-c3ccc(-n4c5ccccc5c5ccccc54)cc3)c3oc4ccccc4c23)cc1]

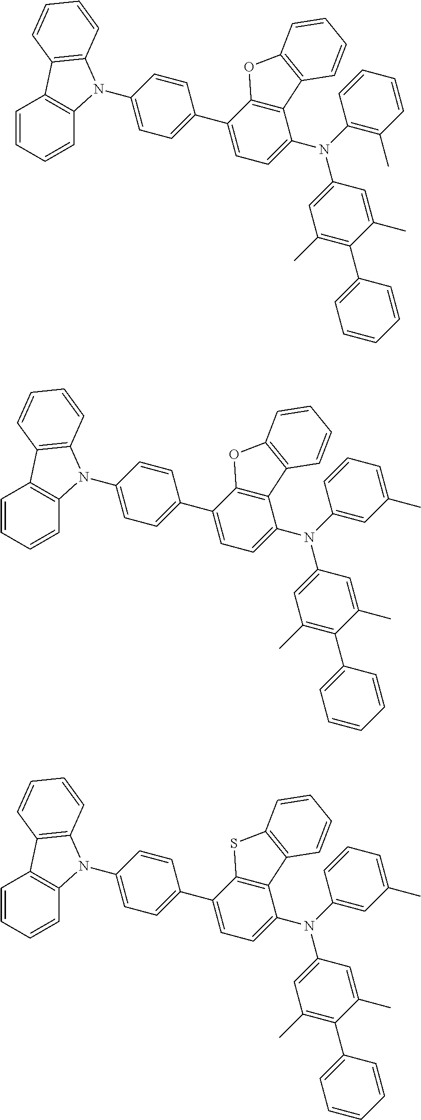 [SMILES: Cc1ccccc1N(c1cc(C)c(-c2ccccc2)c(C)c1)c1ccc(-c2ccc(-n3c4ccccc4c4ccccc43)cc2)c2oc3ccccc3c12 ; Cc1cccc(N(c2cc(C)c(-c3ccccc3)c(C)c2)c2ccc(-c3ccc(-n4c5ccccc5c5ccccc54)cc3)c3oc4ccccc4c23)c1 ; Cc1cccc(N(c2cc(C)c(-c3ccccc3)c(C)c2)c2ccc(-c3ccc(-n4c5ccccc5c5ccccc54)cc3)c3sc4ccccc4c23)c1]

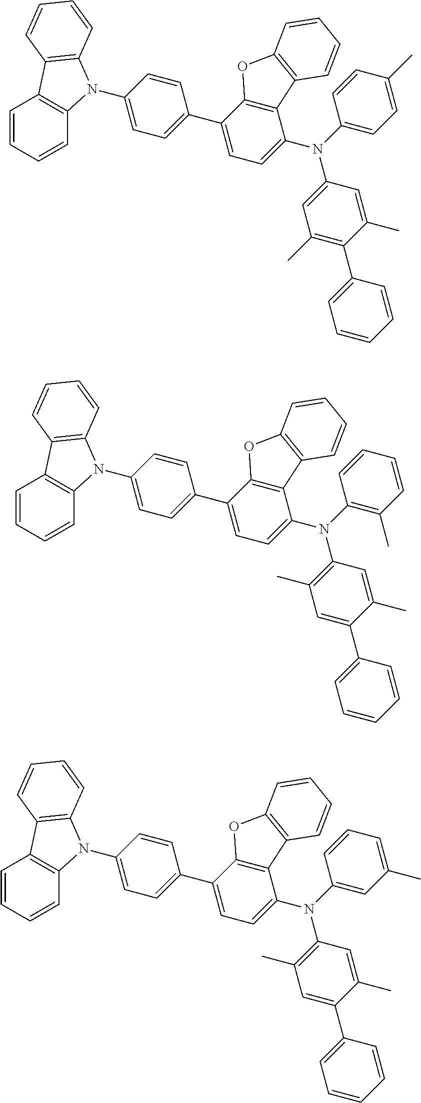 [SMILES: Cc1ccc(N(c2cc(C)c(-c3ccccc3)c(C)c2)c2ccc(-c3ccc(-n4c5ccccc5c5ccccc54)cc3)c3oc4ccccc4c23)cc1 ; Cc1cc(N(c2ccccc2C)c2ccc(-c3ccc(-n4c5ccccc5c5ccccc54)cc3)c3oc4ccccc4c23)c(C)cc1-c1ccccc1 ; Cc1cccc(N(c2cc(C)c(-c3ccccc3)cc2C)c2ccc(-c3ccc(-n4c5ccccc5c5ccccc54)cc3)c3oc4ccccc4c23)c1]

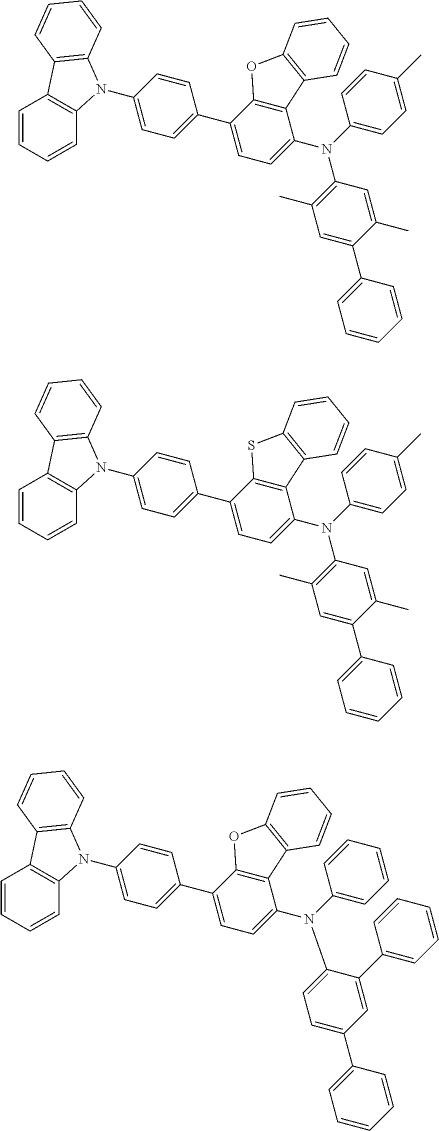 [SMILES: Cc1ccc(N(c2cc(C)c(-c3ccccc3)cc2C)c2ccc(-c3ccc(-n4c5ccccc5c5ccccc54)cc3)c3oc4ccccc4c23)cc1 ; Cc1ccc(N(c2cc(C)c(-c3ccccc3)cc2C)c2ccc(-c3ccc(-n4c5ccccc5c5ccccc54)cc3)c3sc4ccccc4c23)cc1 ; c1ccc(-c2ccc(N(c3ccccc3)c3ccc(-c4ccc(-n5c6ccccc6c6ccccc65)cc4)c4oc5ccccc5c34)c(-c3ccccc3)c2)cc1]

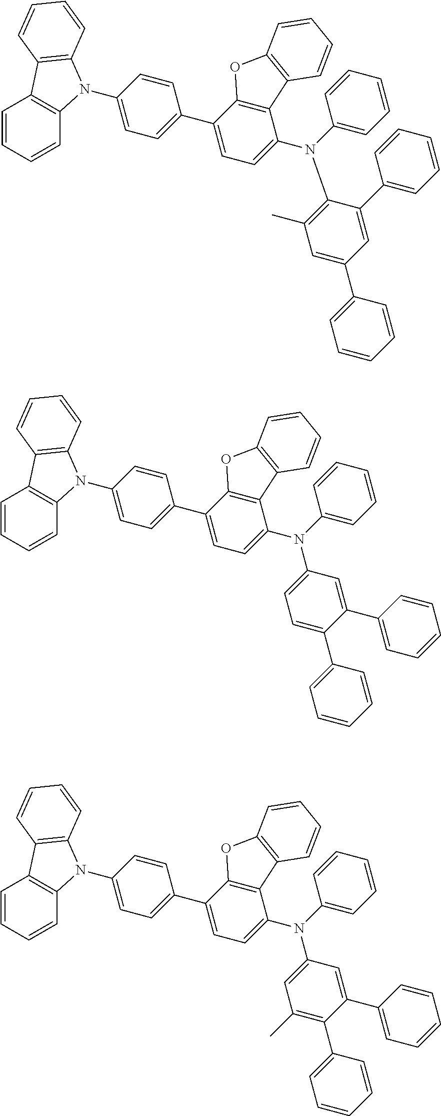 [SMILES: Cc1cc(-c2ccccc2)cc(-c2ccccc2)c1N(c1ccccc1)c1ccc(-c2ccc(-n3c4ccccc4c4ccccc43)cc2)c2oc3ccccc3c12 ; c1ccc(-c2ccc(N(c3ccccc3)c3ccc(-c4ccc(-n5c6ccccc6c6ccccc65)cc4)c4oc5ccccc5c34)cc2-c2ccccc2)cc1 ; Cc1cc(N(c2ccccc2)c2ccc(-c3ccc(-n4c5ccccc5c5ccccc54)cc3)c3oc4ccccc4c23)cc(-c2ccccc2)c1-c1ccccc1]

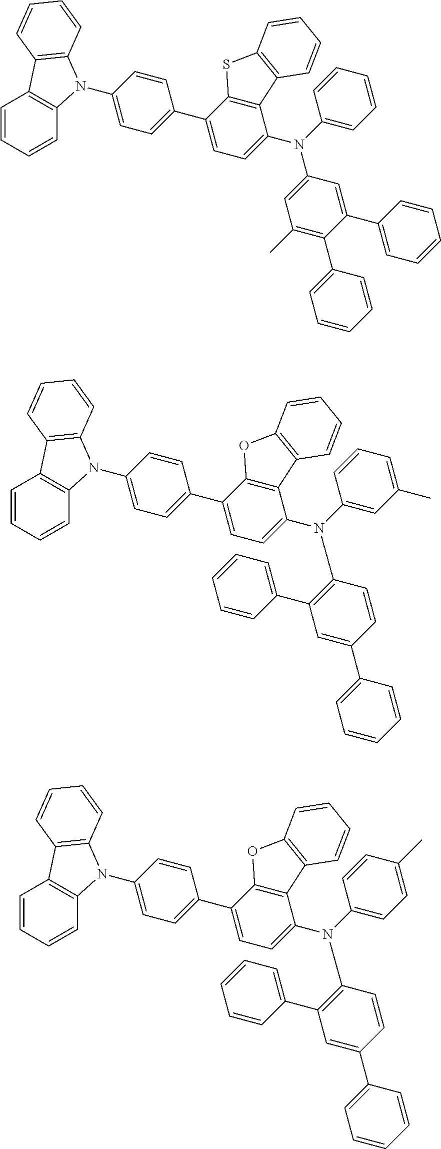 [SMILES: Cc1cc(N(c2ccccc2)c2ccc(-c3ccc(-n4c5ccccc5c5ccccc54)cc3)c3sc4ccccc4c23)cc(-c2ccccc2)c1-c1ccccc1 ; Cc1cccc(N(c2ccc(-c3ccccc3)cc2-c2ccccc2)c2ccc(-c3ccc(-n4c5ccccc5c5ccccc54)cc3)c3oc4ccccc4c23)c1 ; Cc1ccc(N(c2ccc(-c3ccccc3)cc2-c2ccccc2)c2ccc(-c3ccc(-n4c5ccccc5c5ccccc54)cc3)c3oc4ccccc4c23)cc1]

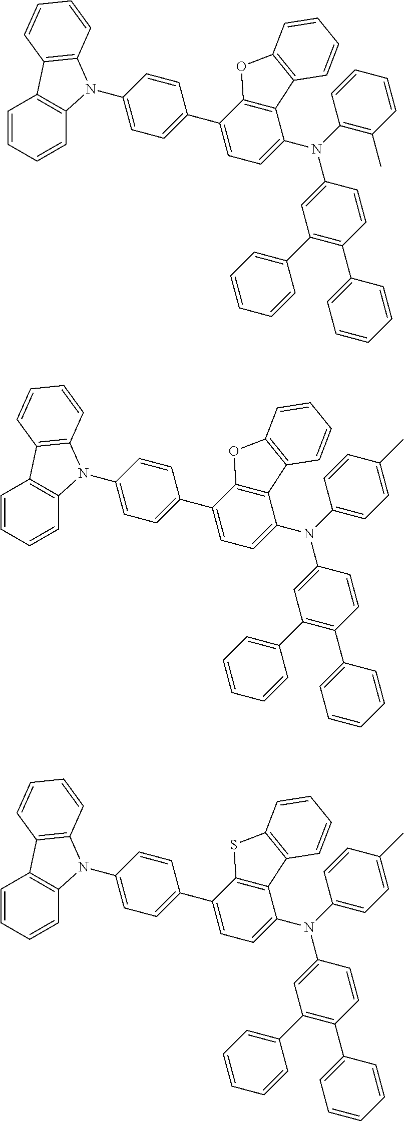 [SMILES: Cc1ccccc1N(c1ccc(-c2ccccc2)c(-c2ccccc2)c1)c1ccc(-c2ccc(-n3c4ccccc4c4ccccc43)cc2)c2oc3ccccc3c12 ; Cc1ccc(N(c2ccc(-c3ccccc3)c(-c3ccccc3)c2)c2ccc(-c3ccc(-n4c5ccccc5c5ccccc54)cc3)c3oc4ccccc4c23)cc1 ; Cc1ccc(N(c2ccc(-c3ccccc3)c(-c3ccccc3)c2)c2ccc(-c3ccc(-n4c5ccccc5c5ccccc54)cc3)c3sc4ccccc4c23)cc1]

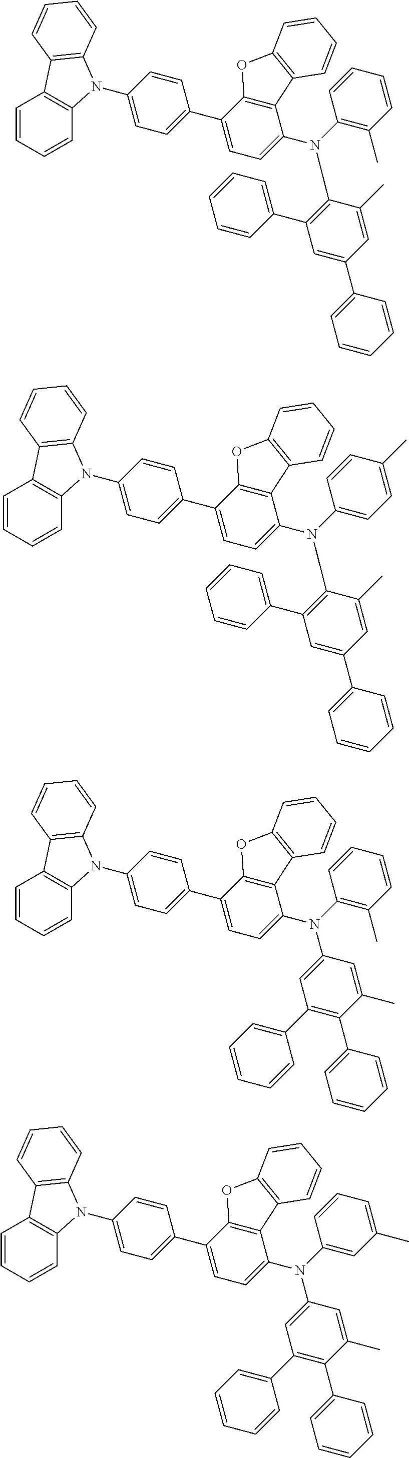 [SMILES: Cc1ccccc1N(c1c(C)cc(-c2ccccc2)cc1-c1ccccc1)c1ccc(-c2ccc(-n3c4ccccc4c4ccccc43)cc2)c2oc3ccccc3c12 ; Cc1ccc(N(c2c(C)cc(-c3ccccc3)cc2-c2ccccc2)c2ccc(-c3ccc(-n4c5ccccc5c5ccccc54)cc3)c3oc4ccccc4c23)cc1 ; Cc1ccccc1N(c1cc(C)c(-c2ccccc2)c(-c2ccccc2)c1)c1ccc(-c2ccc(-n3c4ccccc4c4ccccc43)cc2)c2oc3ccccc3c12 ; Cc1cccc(N(c2cc(C)c(-c3ccccc3)c(-c3ccccc3)c2)c2ccc(-c3ccc(-n4c5ccccc5c5ccccc54)cc3)c3oc4ccccc4c23)c1]

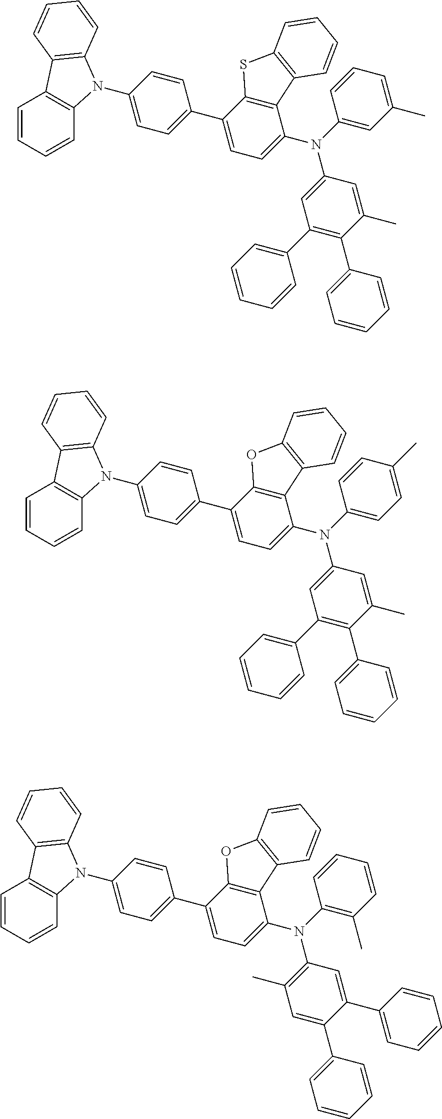 [SMILES: Cc1cccc(N(c2cc(C)c(-c3ccccc3)c(-c3ccccc3)c2)c2ccc(-c3ccc(-n4c5ccccc5c5ccccc54)cc3)c3sc4ccccc4c23)c1 ; Cc1ccc(N(c2cc(C)c(-c3ccccc3)c(-c3ccccc3)c2)c2ccc(-c3ccc(-n4c5ccccc5c5ccccc54)cc3)c3oc4ccccc4c23)cc1 ; Cc1ccccc1N(c1cc(-c2ccccc2)c(-c2ccccc2)cc1C)c1ccc(-c2ccc(-n3c4ccccc4c4ccccc43)cc2)c2oc3ccccc3c12]

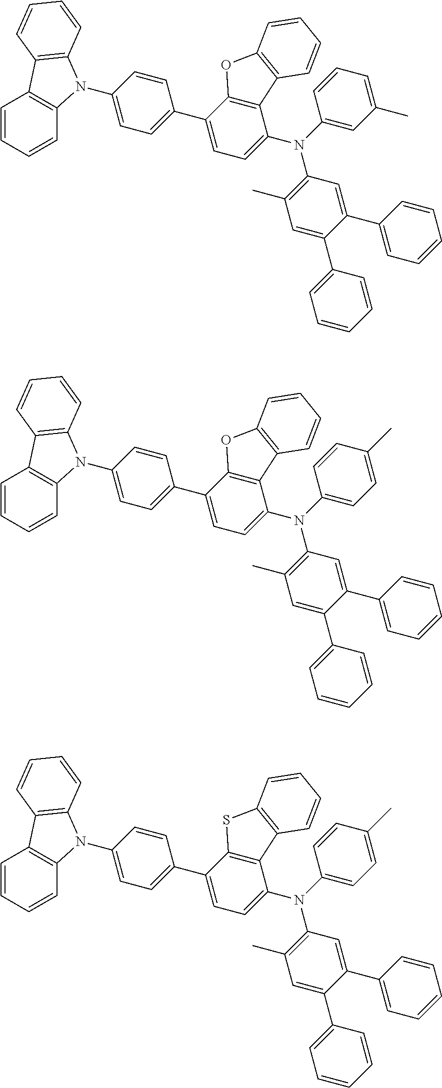 [SMILES: Cc1cccc(N(c2cc(-c3ccccc3)c(-c3ccccc3)cc2C)c2ccc(-c3ccc(-n4c5ccccc5c5ccccc54)cc3)c3oc4ccccc4c23)c1 ; Cc1ccc(N(c2cc(-c3ccccc3)c(-c3ccccc3)cc2C)c2ccc(-c3ccc(-n4c5ccccc5c5ccccc54)cc3)c3oc4ccccc4c23)cc1 ; Cc1ccc(N(c2cc(-c3ccccc3)c(-c3ccccc3)cc2C)c2ccc(-c3ccc(-n4c5ccccc5c5ccccc54)cc3)c3sc4ccccc4c23)cc1]

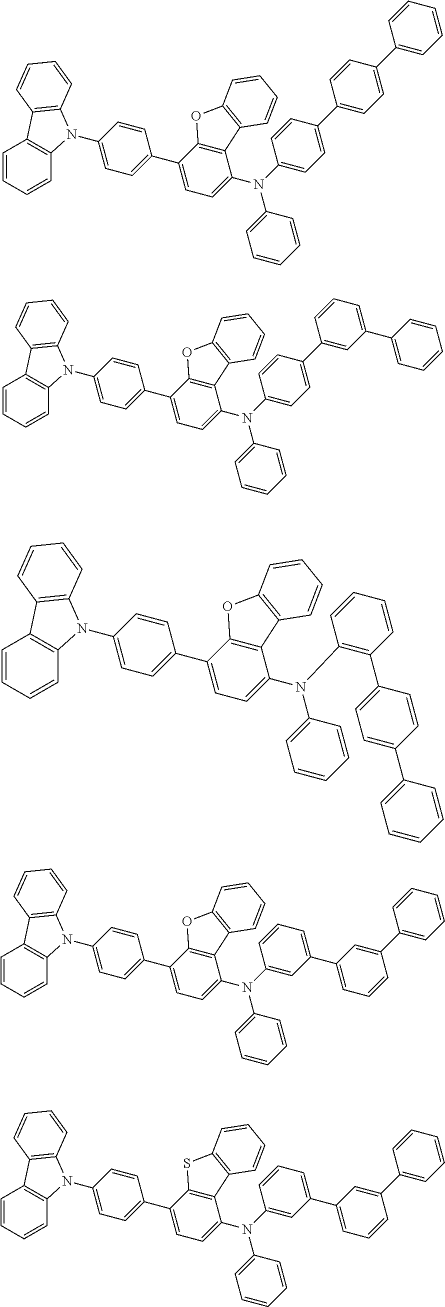 [SMILES: c1ccc(-c2ccc(-c3ccc(N(c4ccccc4)c4ccc(-c5ccc(-n6c7ccccc7c7ccccc76)cc5)c5oc6ccccc6c45)cc3)cc2)cc1 ; c1ccc(-c2cccc(-c3ccc(N(c4ccccc4)c4ccc(-c5ccc(-n6c7ccccc7c7ccccc76)cc5)c5oc6ccccc6c45)cc3)c2)cc1 ; c1ccc(-c2ccc(-c3ccccc3N(c3ccccc3)c3ccc(-c4ccc(-n5c6ccccc6c6ccccc65)cc4)c4oc5ccccc5c34)cc2)cc1 ; c1ccc(-c2cccc(-c3cccc(N(c4ccccc4)c4ccc(-c5ccc(-n6c7ccccc7c7ccccc76)cc5)c5oc6ccccc6c45)c3)c2)cc1 ; c1ccc(-c2cccc(-c3cccc(N(c4ccccc4)c4ccc(-c5ccc(-n6c7ccccc7c7ccccc76)cc5)c5sc6ccccc6c45)c3)c2)cc1]

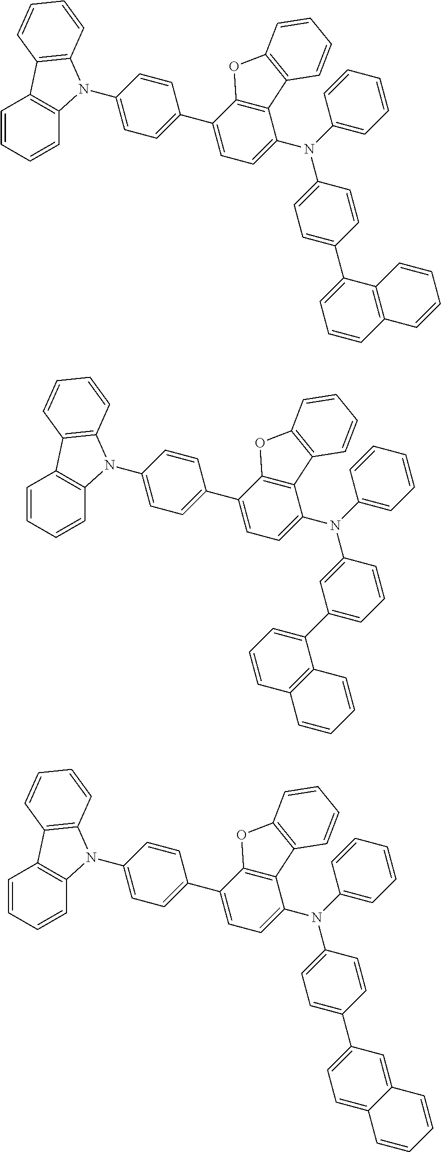 [SMILES: c1ccc(N(c2ccc(-c3cccc4ccccc34)cc2)c2ccc(-c3ccc(-n4c5ccccc5c5ccccc54)cc3)c3oc4ccccc4c23)cc1 ; c1ccc(N(c2cccc(-c3cccc4ccccc34)c2)c2ccc(-c3ccc(-n4c5ccccc5c5ccccc54)cc3)c3oc4ccccc4c23)cc1 ; c1ccc(N(c2ccc(-c3ccc4ccccc4c3)cc2)c2ccc(-c3ccc(-n4c5ccccc5c5ccccc54)cc3)c3oc4ccccc4c23)cc1]

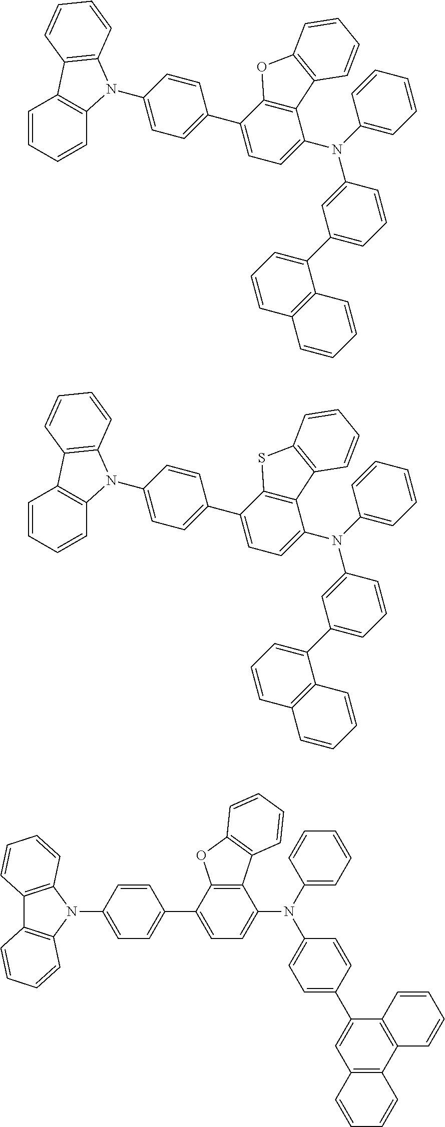 [SMILES: c1ccc(N(c2cccc(-c3cccc4ccccc34)c2)c2ccc(-c3ccc(-n4c5ccccc5c5ccccc54)cc3)c3oc4ccccc4c23)cc1 ; c1ccc(N(c2cccc(-c3cccc4ccccc34)c2)c2ccc(-c3ccc(-n4c5ccccc5c5ccccc54)cc3)c3sc4ccccc4c23)cc1 ; c1ccc(N(c2ccc(-c3cc4ccccc4c4ccccc34)cc2)c2ccc(-c3ccc(-n4c5ccccc5c5ccccc54)cc3)c3oc4ccccc4c23)cc1]

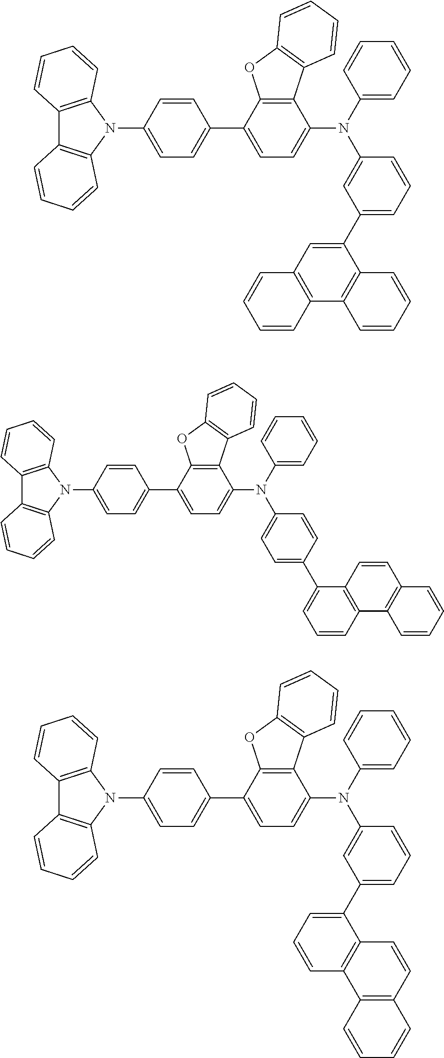

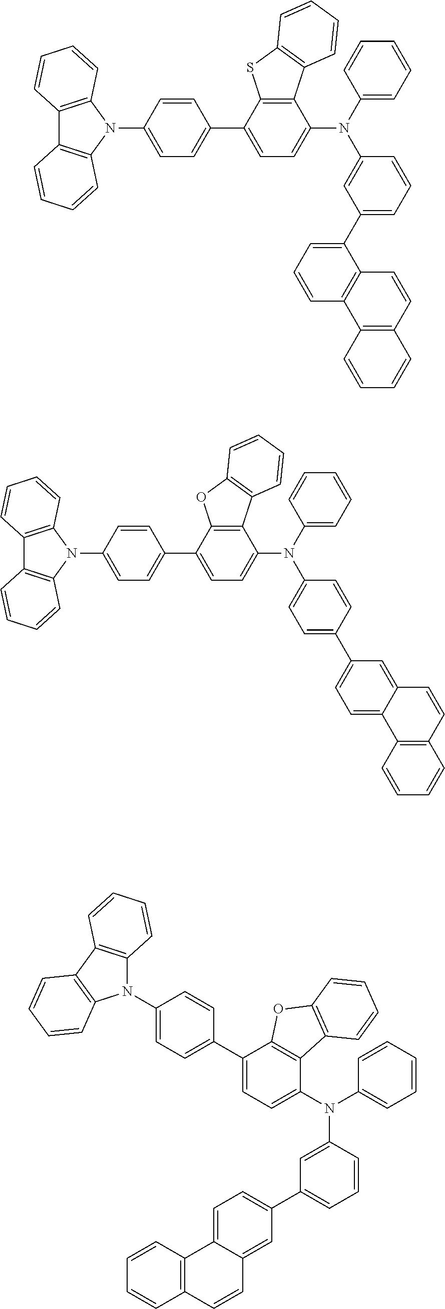 [SMILES: c1ccc(N(c2cccc(-c3cccc4c3ccc3ccccc34)c2)c2ccc(-c3ccc(-n4c5ccccc5c5ccccc54)cc3)c3sc4ccccc4c23)cc1 ; c1ccc(N(c2ccc(-c3ccc4c(ccc5ccccc54)c3)cc2)c2ccc(-c3ccc(-n4c5ccccc5c5ccccc54)cc3)c3oc4ccccc4c23)cc1 ; c1ccc(N(c2cccc(-c3ccc4c(ccc5ccccc54)c3)c2)c2ccc(-c3ccc(-n4c5ccccc5c5ccccc54)cc3)c3oc4ccccc4c23)cc1]

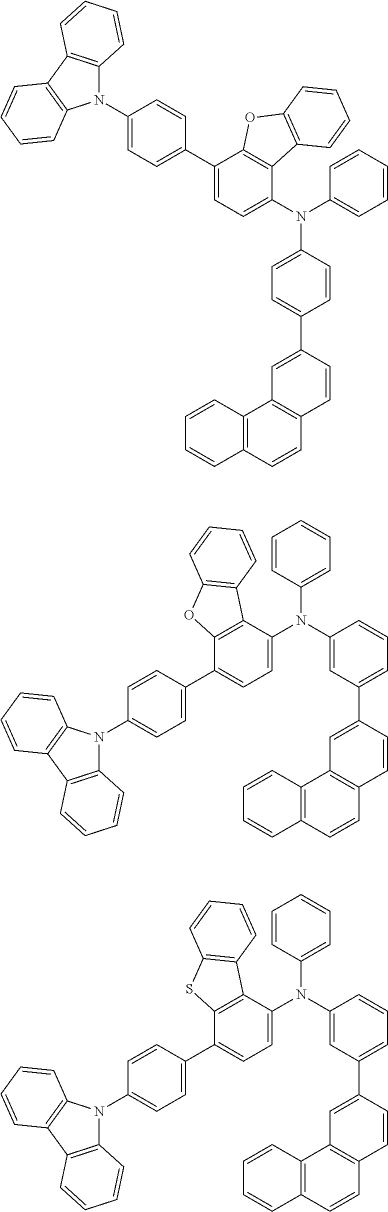 [SMILES: c1ccc(N(c2ccc(-c3ccc4ccc5ccccc5c4c3)cc2)c2ccc(-c3ccc(-n4c5ccccc5c5ccccc54)cc3)c3oc4ccccc4c23)cc1 ; c1ccc(N(c2cccc(-c3ccc4ccc5ccccc5c4c3)c2)c2ccc(-c3ccc(-n4c5ccccc5c5ccccc54)cc3)c3oc4ccccc4c23)cc1 ; c1ccc(N(c2cccc(-c3ccc4ccc5ccccc5c4c3)c2)c2ccc(-c3ccc(-n4c5ccccc5c5ccccc54)cc3)c3sc4ccccc4c23)cc1]

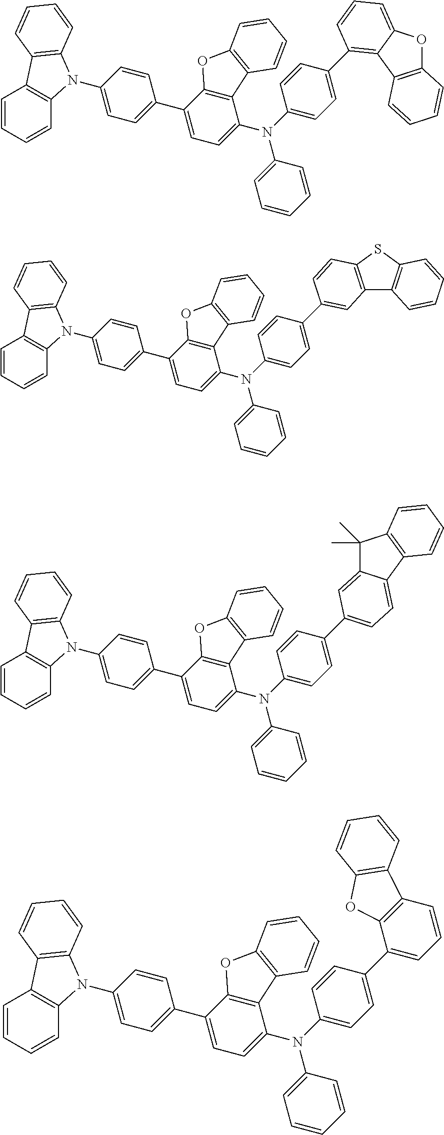 [SMILES: c1ccc(N(c2ccc(-c3cccc4oc5ccccc5c34)cc2)c2ccc(-c3ccc(-n4c5ccccc5c5ccccc54)cc3)c3oc4ccccc4c23)cc1 ; c1ccc(N(c2ccc(-c3ccc4sc5ccccc5c4c3)cc2)c2ccc(-c3ccc(-n4c5ccccc5c5ccccc54)cc3)c3oc4ccccc4c23)cc1 ; CC1(C)c2ccccc2-c2ccc(-c3ccc(N(c4ccccc4)c4ccc(-c5ccc(-n6c7ccccc7c7ccccc76)cc5)c5oc6ccccc6c45)cc3)cc21 ; c1ccc(N(c2ccc(-c3cccc4c3oc3ccccc34)cc2)c2ccc(-c3ccc(-n4c5ccccc5c5ccccc54)cc3)c3oc4ccccc4c23)cc1]

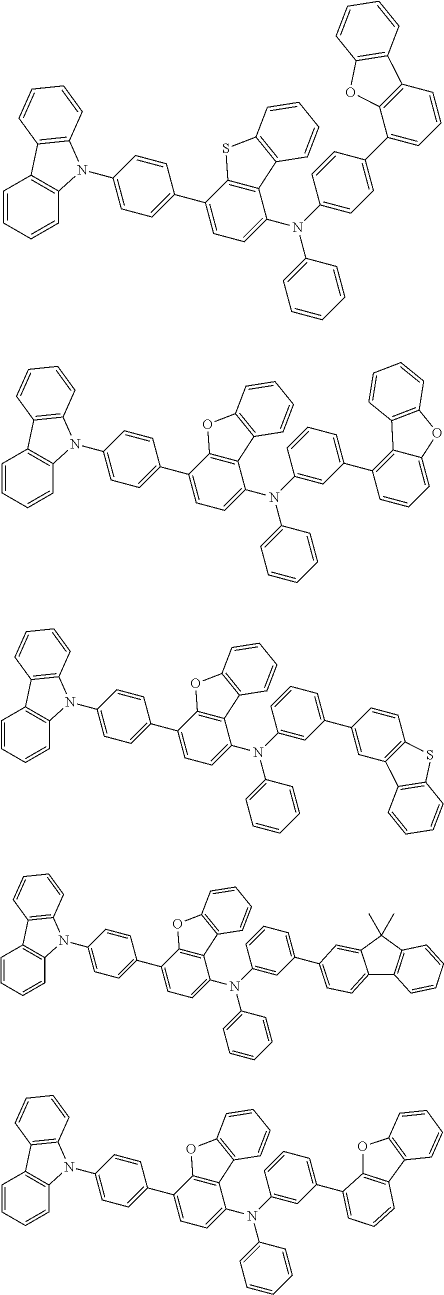 [SMILES: c1ccc(N(c2ccc(-c3cccc4c3oc3ccccc34)cc2)c2ccc(-c3ccc(-n4c5ccccc5c5ccccc54)cc3)c3sc4ccccc4c23)cc1 ; c1ccc(N(c2cccc(-c3cccc4oc5ccccc5c34)c2)c2ccc(-c3ccc(-n4c5ccccc5c5ccccc54)cc3)c3oc4ccccc4c23)cc1 ; c1ccc(N(c2cccc(-c3ccc4sc5ccccc5c4c3)c2)c2ccc(-c3ccc(-n4c5ccccc5c5ccccc54)cc3)c3oc4ccccc4c23)cc1 ; CC1(C)c2ccccc2-c2ccc(-c3cccc(N(c4ccccc4)c4ccc(-c5ccc(-n6c7ccccc7c7ccccc76)cc5)c5oc6ccccc6c45)c3)cc21 ; c1ccc(N(c2cccc(-c3cccc4c3oc3ccccc34)c2)c2ccc(-c3ccc(-n4c5ccccc5c5ccccc54)cc3)c3oc4ccccc4c23)cc1]

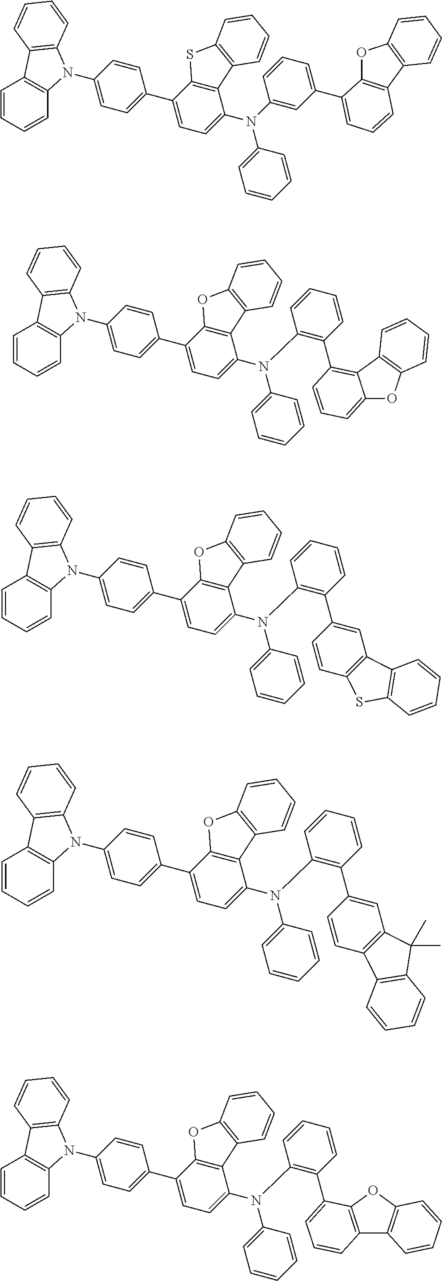 [SMILES: c1ccc(N(c2cccc(-c3cccc4c3oc3ccccc34)c2)c2ccc(-c3ccc(-n4c5ccccc5c5ccccc54)cc3)c3sc4ccccc4c23)cc1 ; c1ccc(N(c2ccccc2-c2cccc3oc4ccccc4c23)c2ccc(-c3ccc(-n4c5ccccc5c5ccccc54)cc3)c3oc4ccccc4c23)cc1 ; c1ccc(N(c2ccccc2-c2ccc3sc4ccccc4c3c2)c2ccc(-c3ccc(-n4c5ccccc5c5ccccc54)cc3)c3oc4ccccc4c23)cc1 ; CC1(C)c2ccccc2-c2ccc(-c3ccccc3N(c3ccccc3)c3ccc(-c4ccc(-n5c6ccccc6c6ccccc65)cc4)c4oc5ccccc5c34)cc21 ; c1ccc(N(c2ccccc2-c2cccc3c2oc2ccccc23)c2ccc(-c3ccc(-n4c5ccccc5c5ccccc54)cc3)c3oc4ccccc4c23)cc1]

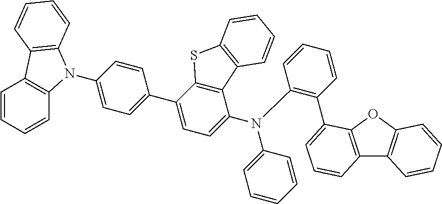 [SMILES: c1ccc(N(c2ccccc2-c2cccc3c2oc2ccccc23)c2ccc(-c3ccc(-n4c5ccccc5c5ccccc54)cc3)c3sc4ccccc4c23)cc1]

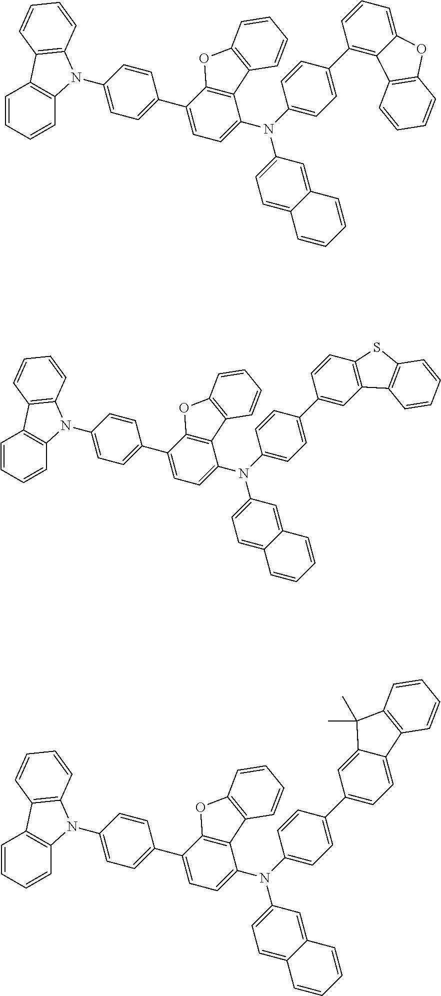 [SMILES: c1ccc2cc(N(c3ccc(-c4cccc5oc6ccccc6c45)cc3)c3ccc(-c4ccc(-n5c6ccccc6c6ccccc65)cc4)c4oc5ccccc5c34)ccc2c1 ; c1ccc2cc(N(c3ccc(-c4ccc5sc6ccccc6c5c4)cc3)c3ccc(-c4ccc(-n5c6ccccc6c6ccccc65)cc4)c4oc5ccccc5c34)ccc2c1 ; CC1(C)c2ccccc2-c2ccc(-c3ccc(N(c4ccc5ccccc5c4)c4ccc(-c5ccc(-n6c7ccccc7c7ccccc76)cc5)c5oc6ccccc6c45)cc3)cc21]

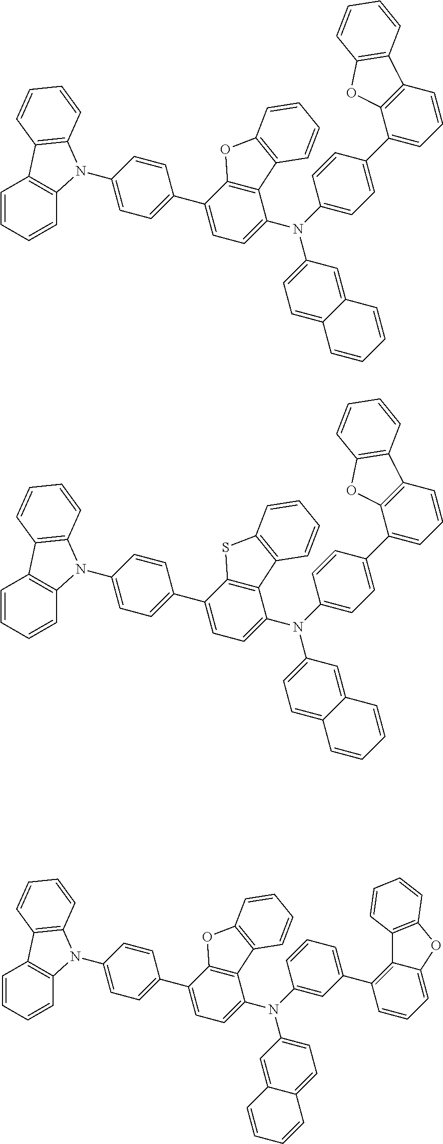

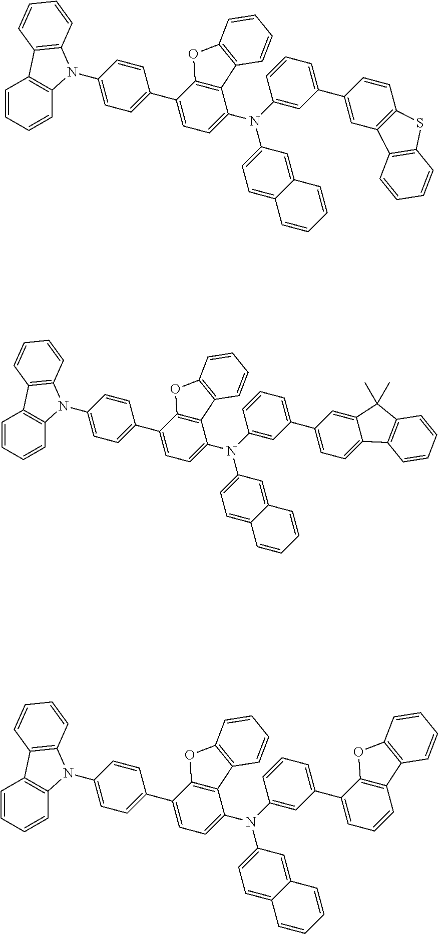 [SMILES: c1cc(-c2ccc3sc4ccccc4c3c2)cc(N(c2ccc3ccccc3c2)c2ccc(-c3ccc(-n4c5ccccc5c5ccccc54)cc3)c3oc4ccccc4c23)c1 ; CC1(C)c2ccccc2-c2ccc(-c3cccc(N(c4ccc5ccccc5c4)c4ccc(-c5ccc(-n6c7ccccc7c7ccccc76)cc5)c5oc6ccccc6c45)c3)cc21 ; c1cc(-c2cccc3c2oc2ccccc23)cc(N(c2ccc3ccccc3c2)c2ccc(-c3ccc(-n4c5ccccc5c5ccccc54)cc3)c3oc4ccccc4c23)c1]

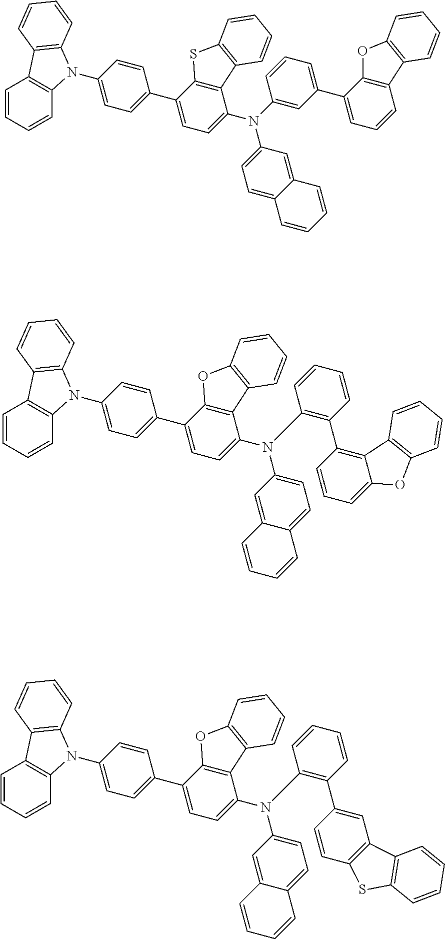 [SMILES: c1cc(-c2cccc3c2oc2ccccc23)cc(N(c2ccc3ccccc3c2)c2ccc(-c3ccc(-n4c5ccccc5c5ccccc54)cc3)c3sc4ccccc4c23)c1 ; c1ccc(N(c2ccc3ccccc3c2)c2ccc(-c3ccc(-n4c5ccccc5c5ccccc54)cc3)c3oc4ccccc4c23)c(-c2cccc3oc4ccccc4c23)c1 ; c1ccc(N(c2ccc3ccccc3c2)c2ccc(-c3ccc(-n4c5ccccc5c5ccccc54)cc3)c3oc4ccccc4c23)c(-c2ccc3sc4ccccc4c3c2)c1]

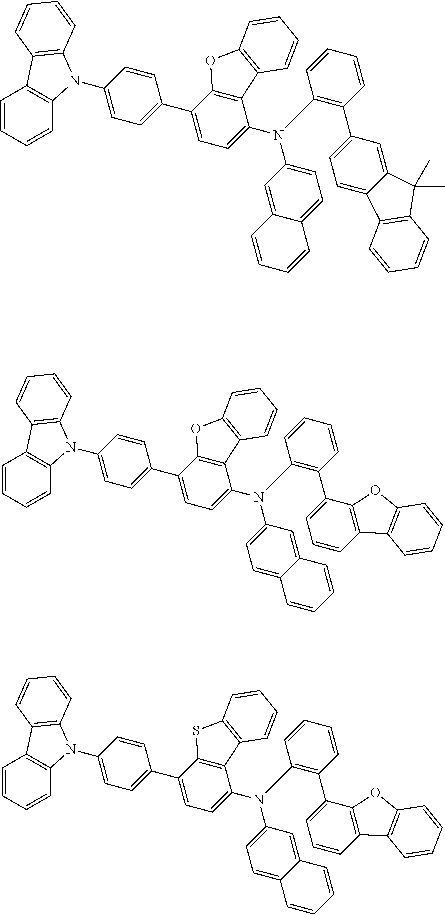 [SMILES: CC1(C)c2ccccc2-c2ccc(-c3ccccc3N(c3ccc4ccccc4c3)c3ccc(-c4ccc(-n5c6ccccc6c6ccccc65)cc4)c4oc5ccccc5c34)cc21 ; c1ccc(N(c2ccc3ccccc3c2)c2ccc(-c3ccc(-n4c5ccccc5c5ccccc54)cc3)c3oc4ccccc4c23)c(-c2cccc3c2oc2ccccc23)c1 ; c1ccc(N(c2ccc3ccccc3c2)c2ccc(-c3ccc(-n4c5ccccc5c5ccccc54)cc3)c3sc4ccccc4c23)c(-c2cccc3c2oc2ccccc23)c1]

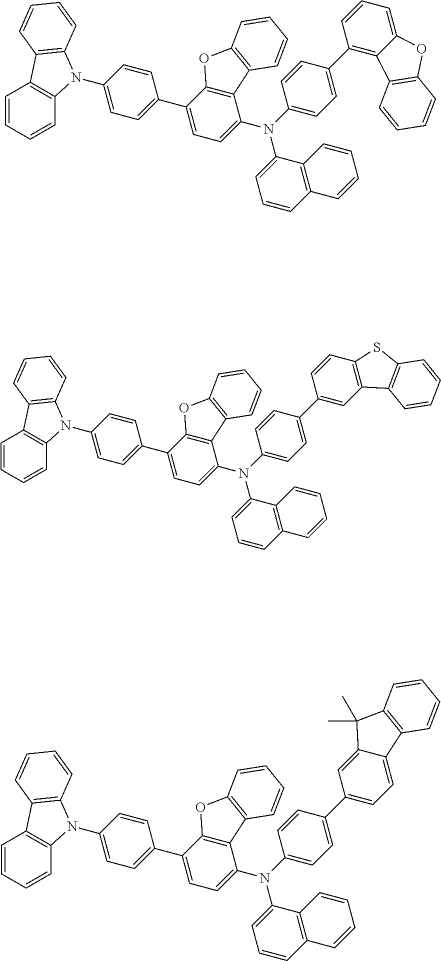 [SMILES: c1ccc2c(N(c3ccc(-c4cccc5oc6ccccc6c45)cc3)c3ccc(-c4ccc(-n5c6ccccc6c6ccccc65)cc4)c4oc5ccccc5c34)cccc2c1 ; c1ccc2c(N(c3ccc(-c4ccc5sc6ccccc6c5c4)cc3)c3ccc(-c4ccc(-n5c6ccccc6c6ccccc65)cc4)c4oc5ccccc5c34)cccc2c1 ; CC1(C)c2ccccc2-c2ccc(-c3ccc(N(c4cccc5ccccc45)c4ccc(-c5ccc(-n6c7ccccc7c7ccccc76)cc5)c5oc6ccccc6c45)cc3)cc21]

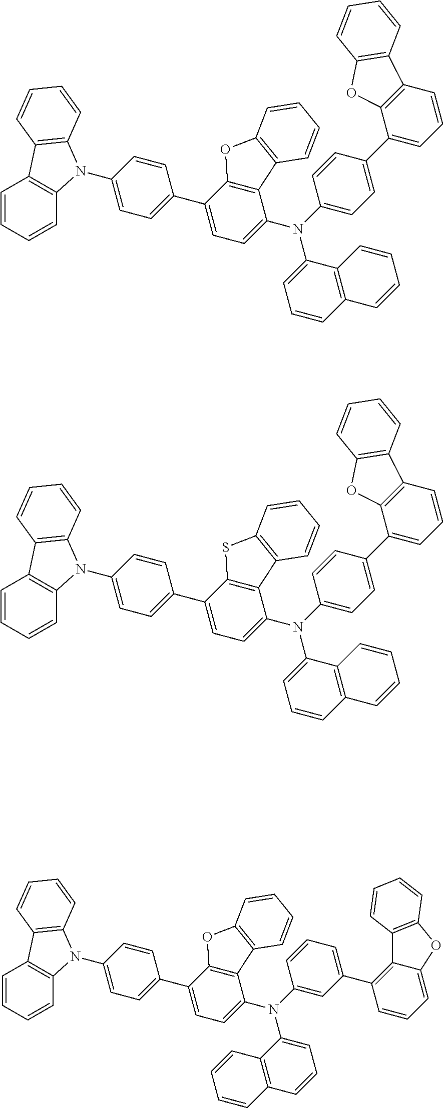 [SMILES: c1ccc2c(N(c3ccc(-c4cccc5c4oc4ccccc45)cc3)c3ccc(-c4ccc(-n5c6ccccc6c6ccccc65)cc4)c4oc5ccccc5c34)cccc2c1 ; c1ccc2c(N(c3ccc(-c4cccc5c4oc4ccccc45)cc3)c3ccc(-c4ccc(-n5c6ccccc6c6ccccc65)cc4)c4sc5ccccc5c34)cccc2c1 ; c1cc(-c2cccc3oc4ccccc4c23)cc(N(c2cccc3ccccc23)c2ccc(-c3ccc(-n4c5ccccc5c5ccccc54)cc3)c3oc4ccccc4c23)c1]

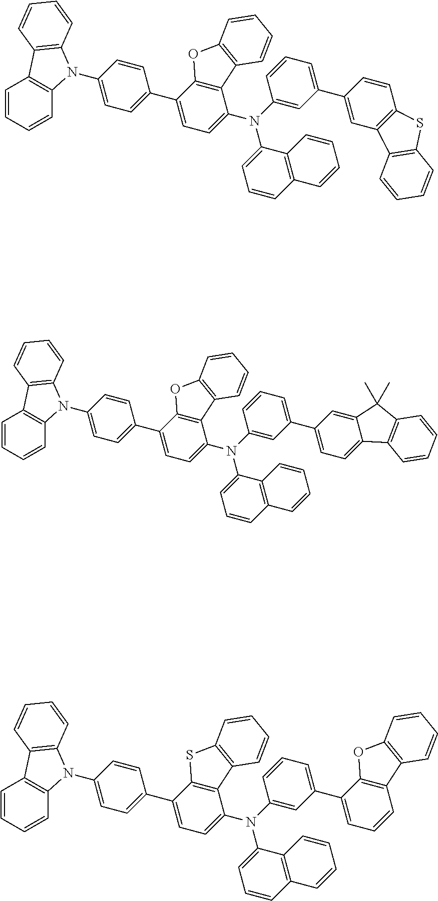 [SMILES: c1cc(-c2ccc3sc4ccccc4c3c2)cc(N(c2cccc3ccccc23)c2ccc(-c3ccc(-n4c5ccccc5c5ccccc54)cc3)c3oc4ccccc4c23)c1 ; CC1(C)c2ccccc2-c2ccc(-c3cccc(N(c4cccc5ccccc45)c4ccc(-c5ccc(-n6c7ccccc7c7ccccc76)cc5)c5oc6ccccc6c45)c3)cc21 ; c1cc(-c2cccc3c2oc2ccccc23)cc(N(c2cccc3ccccc23)c2ccc(-c3ccc(-n4c5ccccc5c5ccccc54)cc3)c3sc4ccccc4c23)c1]

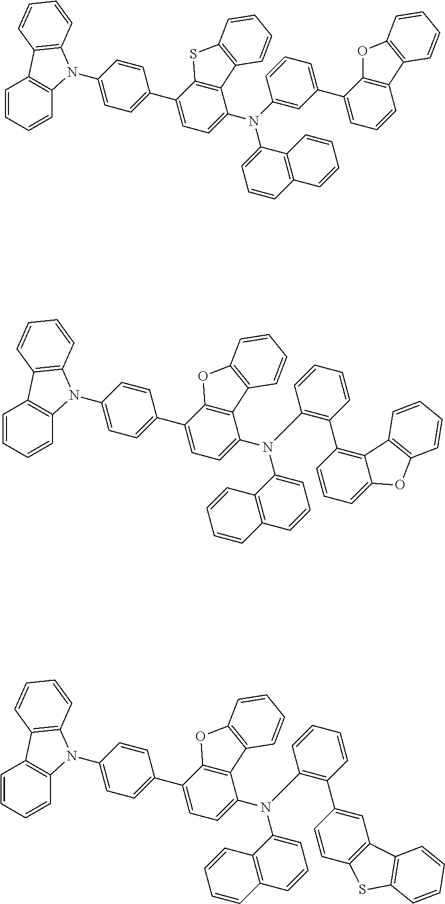 [SMILES: c1cc(-c2cccc3c2oc2ccccc23)cc(N(c2cccc3ccccc23)c2ccc(-c3ccc(-n4c5ccccc5c5ccccc54)cc3)c3sc4ccccc4c23)c1 ; c1ccc(N(c2cccc3ccccc23)c2ccc(-c3ccc(-n4c5ccccc5c5ccccc54)cc3)c3oc4ccccc4c23)c(-c2cccc3oc4ccccc4c23)c1 ; c1ccc(N(c2cccc3ccccc23)c2ccc(-c3ccc(-n4c5ccccc5c5ccccc54)cc3)c3oc4ccccc4c23)c(-c2ccc3sc4ccccc4c3c2)c1]

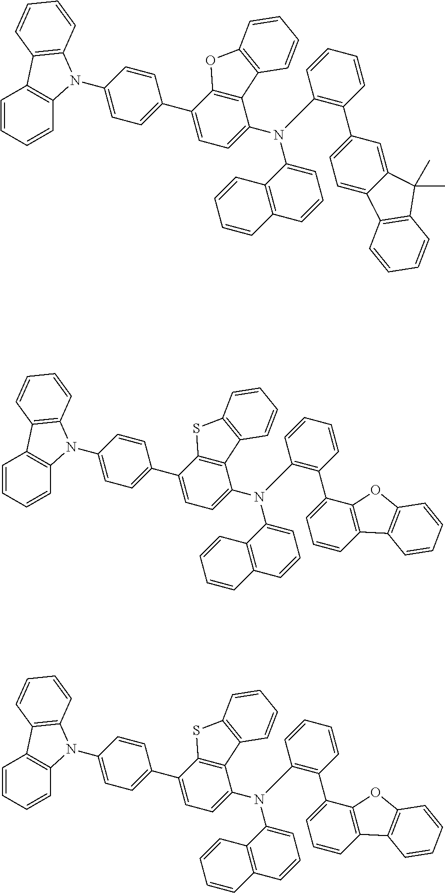 [SMILES: CC1(C)c2ccccc2-c2ccc(-c3ccccc3N(c3cccc4ccccc34)c3ccc(-c4ccc(-n5c6ccccc6c6ccccc65)cc4)c4oc5ccccc5c34)cc21 ; c1ccc(N(c2cccc3ccccc23)c2ccc(-c3ccc(-n4c5ccccc5c5ccccc54)cc3)c3sc4ccccc4c23)c(-c2cccc3c2oc2ccccc23)c1 ; c1ccc(N(c2cccc3ccccc23)c2ccc(-c3ccc(-n4c5ccccc5c5ccccc54)cc3)c3sc4ccccc4c23)c(-c2cccc3c2oc2ccccc23)c1]

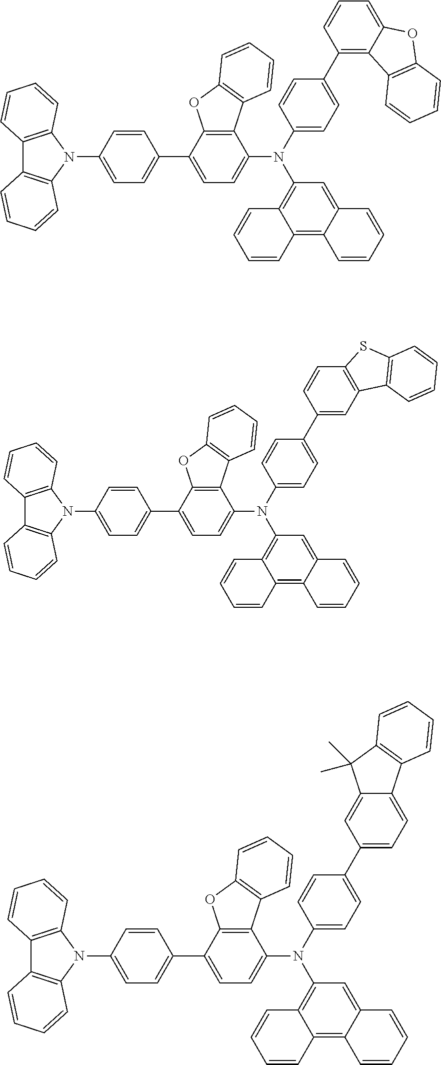 [SMILES: c1ccc2c(c1)cc(N(c1ccc(-c3cccc4oc5ccccc5c34)cc1)c1ccc(-c3ccc(-n4c5ccccc5c5ccccc54)cc3)c3oc4ccccc4c13)c1ccccc12 ; c1ccc2c(c1)cc(N(c1ccc(-c3ccc4sc5ccccc5c4c3)cc1)c1ccc(-c3ccc(-n4c5ccccc5c5ccccc54)cc3)c3oc4ccccc4c13)c1ccccc12 ; CC1(C)c2ccccc2-c2ccc(-c3ccc(N(c4cc5ccccc5c5ccccc45)c4ccc(-c5ccc(-n6c7ccccc7c7ccccc76)cc5)c5oc6ccccc6c45)cc3)cc21]

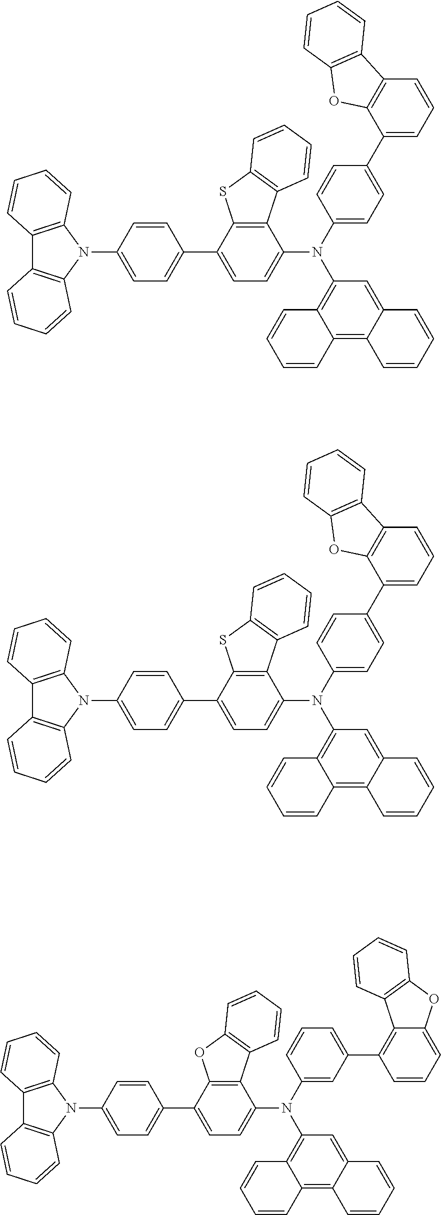 [SMILES: c1ccc2c(c1)cc(N(c1ccc(-c3cccc4c3oc3ccccc34)cc1)c1ccc(-c3ccc(-n4c5ccccc5c5ccccc54)cc3)c3sc4ccccc4c13)c1ccccc12 ; c1ccc2c(c1)cc(N(c1ccc(-c3cccc4c3oc3ccccc34)cc1)c1ccc(-c3ccc(-n4c5ccccc5c5ccccc54)cc3)c3sc4ccccc4c13)c1ccccc12 ; c1cc(-c2cccc3oc4ccccc4c23)cc(N(c2cc3ccccc3c3ccccc23)c2ccc(-c3ccc(-n4c5ccccc5c5ccccc54)cc3)c3oc4ccccc4c23)c1]

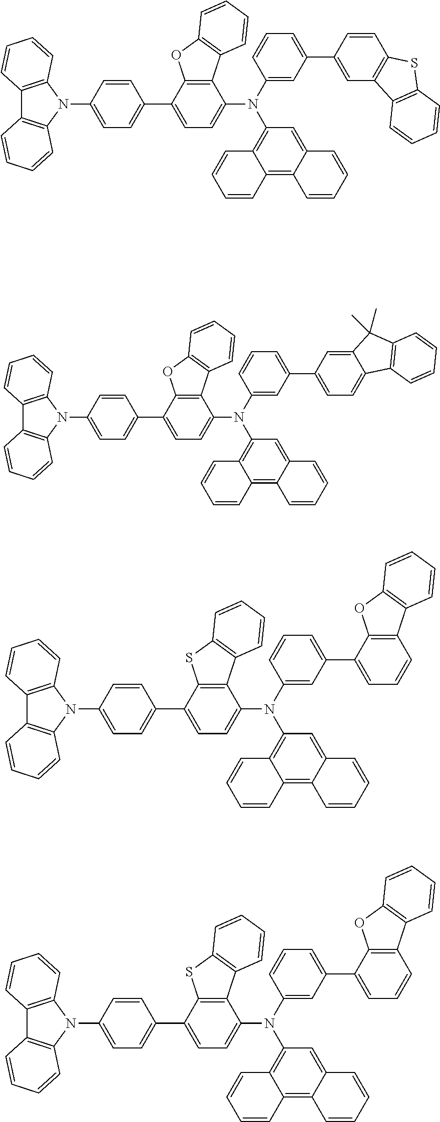 [SMILES: c1cc(-c2ccc3sc4ccccc4c3c2)cc(N(c2cc3ccccc3c3ccccc23)c2ccc(-c3ccc(-n4c5ccccc5c5ccccc54)cc3)c3oc4ccccc4c23)c1 ; CC1(C)c2ccccc2-c2ccc(-c3cccc(N(c4cc5ccccc5c5ccccc45)c4ccc(-c5ccc(-n6c7ccccc7c7ccccc76)cc5)c5oc6ccccc6c45)c3)cc21 ; c1cc(-c2cccc3c2oc2ccccc23)cc(N(c2cc3ccccc3c3ccccc23)c2ccc(-c3ccc(-n4c5ccccc5c5ccccc54)cc3)c3sc4ccccc4c23)c1 ; c1cc(-c2cccc3c2oc2ccccc23)cc(N(c2cc3ccccc3c3ccccc23)c2ccc(-c3ccc(-n4c5ccccc5c5ccccc54)cc3)c3sc4ccccc4c23)c1]

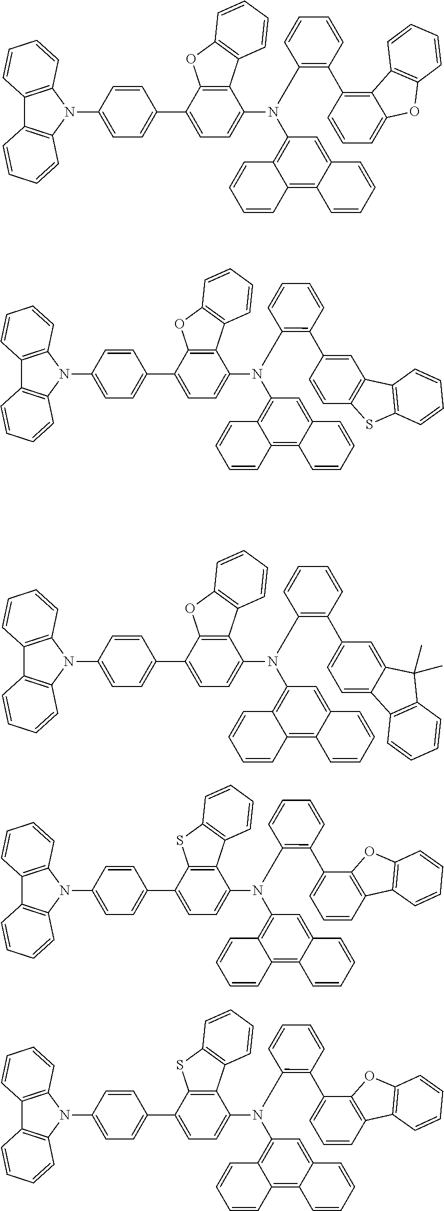

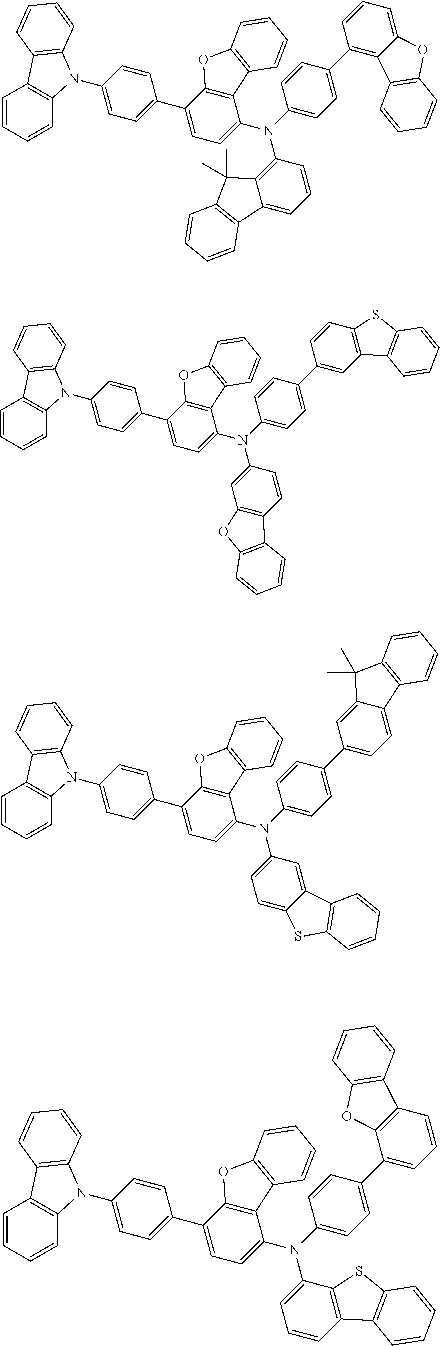 [SMILES: CC1(C)c2ccccc2-c2cccc(N(c3ccc(-c4cccc5oc6ccccc6c45)cc3)c3ccc(-c4ccc(-n5c6ccccc6c6ccccc65)cc4)c4oc5ccccc5c34)c21 ; c1ccc2c(c1)oc1cc(N(c3ccc(-c4ccc5sc6ccccc6c5c4)cc3)c3ccc(-c4ccc(-n5c6ccccc6c6ccccc65)cc4)c4oc5ccccc5c34)ccc12 ; CC1(C)c2ccccc2-c2ccc(-c3ccc(N(c4ccc5sc6ccccc6c5c4)c4ccc(-c5ccc(-n6c7ccccc7c7ccccc76)cc5)c5oc6ccccc6c45)cc3)cc21 ; c1ccc2c(c1)oc1c(-c3ccc(N(c4cccc5c4sc4ccccc45)c4ccc(-c5ccc(-n6c7ccccc7c7ccccc76)cc5)c5oc6ccccc6c45)cc3)cccc12]

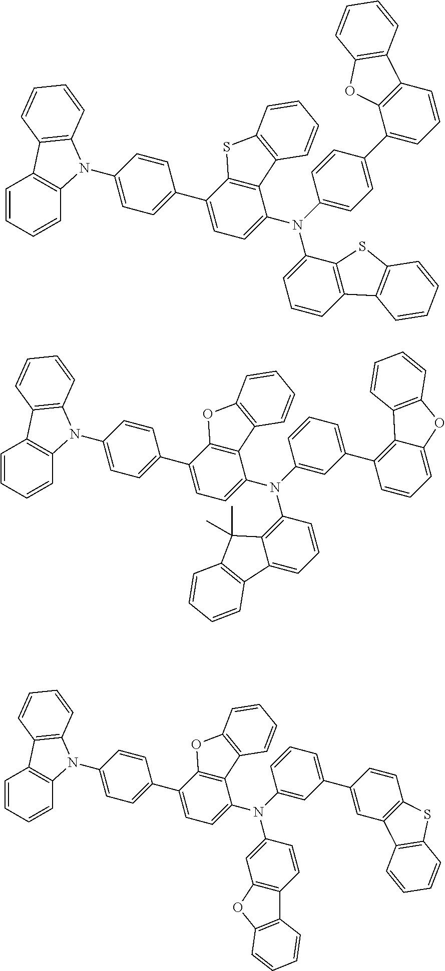

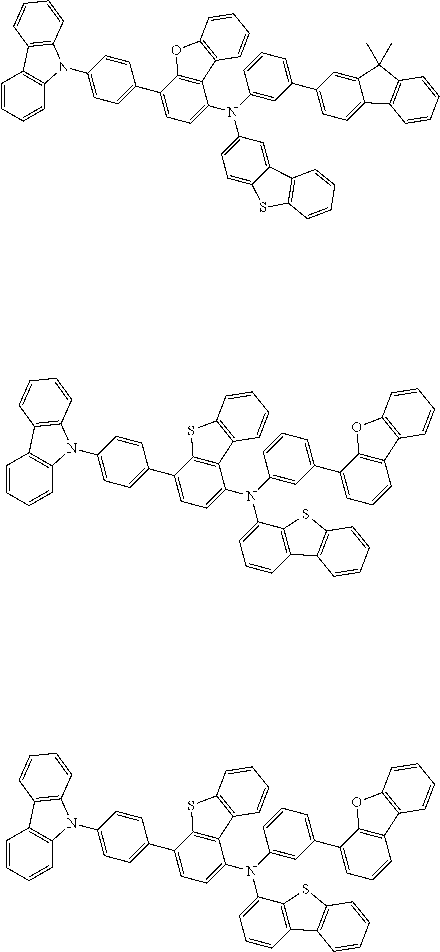 [SMILES: CC1(C)c2ccccc2-c2ccc(-c3cccc(N(c4ccc5sc6ccccc6c5c4)c4ccc(-c5ccc(-n6c7ccccc7c7ccccc76)cc5)c5oc6ccccc6c45)c3)cc21 ; c1cc(-c2cccc3c2oc2ccccc23)cc(N(c2cccc3c2sc2ccccc23)c2ccc(-c3ccc(-n4c5ccccc5c5ccccc54)cc3)c3sc4ccccc4c23)c1 ; c1cc(-c2cccc3c2oc2ccccc23)cc(N(c2cccc3c2sc2ccccc23)c2ccc(-c3ccc(-n4c5ccccc5c5ccccc54)cc3)c3sc4ccccc4c23)c1]

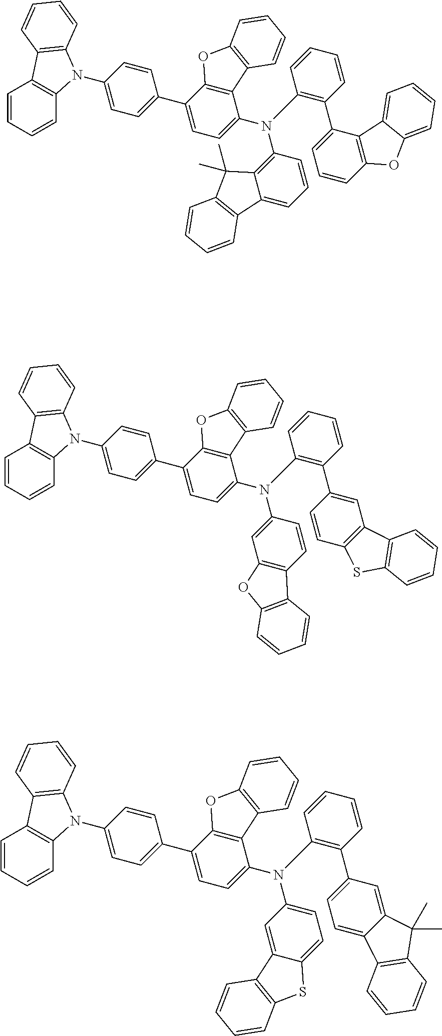 [SMILES: CC1(C)c2ccccc2-c2cccc(N(c3ccccc3-c3cccc4oc5ccccc5c34)c3ccc(-c4ccc(-n5c6ccccc6c6ccccc65)cc4)c4oc5ccccc5c34)c21 ; c1ccc(N(c2ccc3c(c2)oc2ccccc23)c2ccc(-c3ccc(-n4c5ccccc5c5ccccc54)cc3)c3oc4ccccc4c23)c(-c2ccc3sc4ccccc4c3c2)c1 ; CC1(C)c2ccccc2-c2ccc(-c3ccccc3N(c3ccc4sc5ccccc5c4c3)c3ccc(-c4ccc(-n5c6ccccc6c6ccccc65)cc4)c4oc5ccccc5c34)cc21]

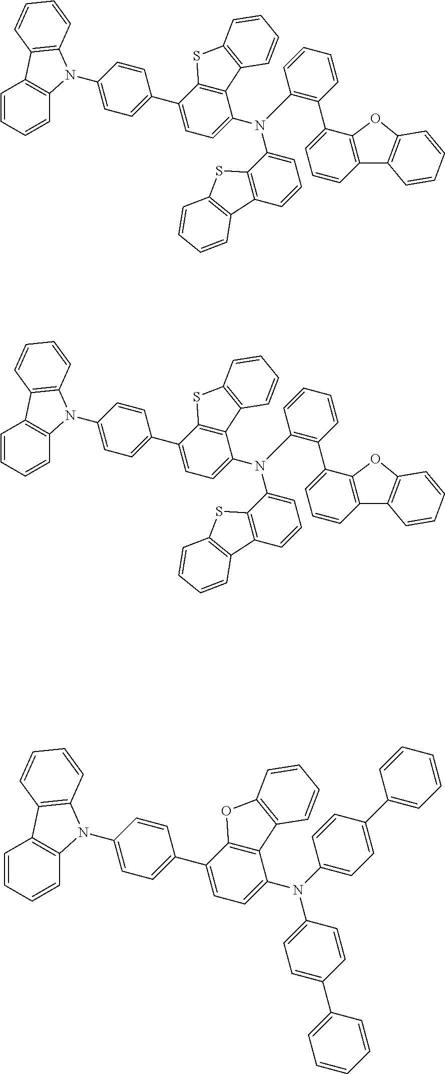 [SMILES: c1ccc(N(c2cccc3c2sc2ccccc23)c2ccc(-c3ccc(-n4c5ccccc5c5ccccc54)cc3)c3sc4ccccc4c23)c(-c2cccc3c2oc2ccccc23)c1 ; c1ccc(N(c2cccc3c2sc2ccccc23)c2ccc(-c3ccc(-n4c5ccccc5c5ccccc54)cc3)c3sc4ccccc4c23)c(-c2cccc3c2oc2ccccc23)c1 ; c1ccc(-c2ccc(N(c3ccc(-c4ccccc4)cc3)c3ccc(-c4ccc(-n5c6ccccc6c6ccccc65)cc4)c4oc5ccccc5c34)cc2)cc1]

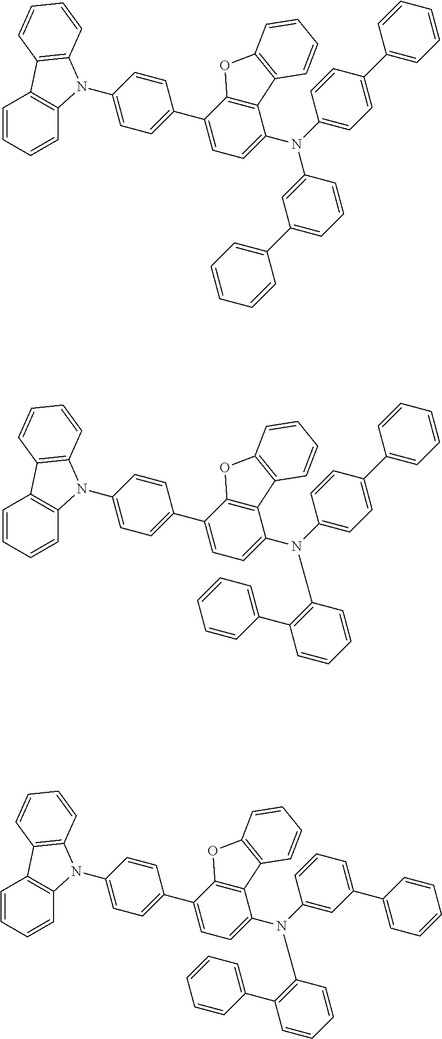 [SMILES: c1ccc(-c2ccc(N(c3cccc(-c4ccccc4)c3)c3ccc(-c4ccc(-n5c6ccccc6c6ccccc65)cc4)c4oc5ccccc5c34)cc2)cc1 ; c1ccc(-c2ccc(N(c3ccccc3-c3ccccc3)c3ccc(-c4ccc(-n5c6ccccc6c6ccccc65)cc4)c4oc5ccccc5c34)cc2)cc1 ; c1ccc(-c2cccc(N(c3ccccc3-c3ccccc3)c3ccc(-c4ccc(-n5c6ccccc6c6ccccc65)cc4)c4oc5ccccc5c34)c2)cc1]

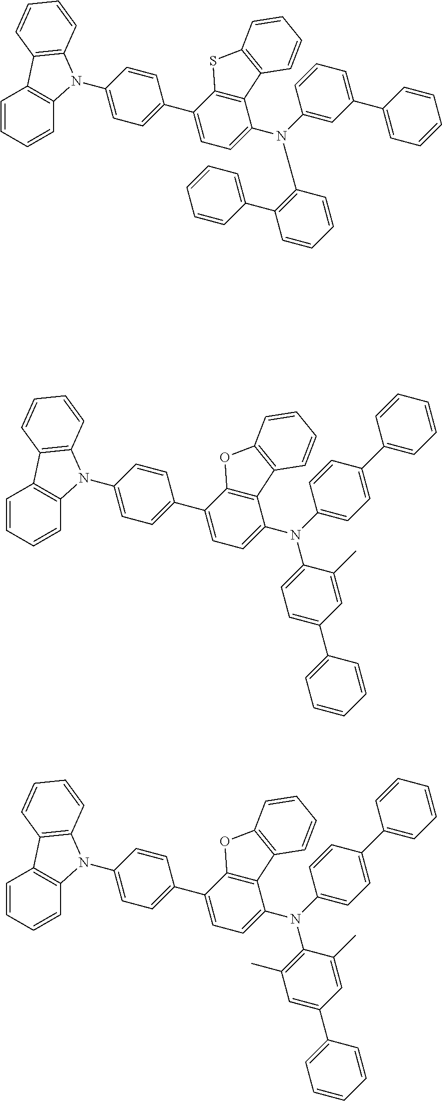 [SMILES: c1ccc(-c2cccc(N(c3ccccc3-c3ccccc3)c3ccc(-c4ccc(-n5c6ccccc6c6ccccc65)cc4)c4sc5ccccc5c34)c2)cc1 ; Cc1cc(-c2ccccc2)ccc1N(c1ccc(-c2ccccc2)cc1)c1ccc(-c2ccc(-n3c4ccccc4c4ccccc43)cc2)c2oc3ccccc3c12 ; Cc1cc(-c2ccccc2)cc(C)c1N(c1ccc(-c2ccccc2)cc1)c1ccc(-c2ccc(-n3c4ccccc4c4ccccc43)cc2)c2oc3ccccc3c12]

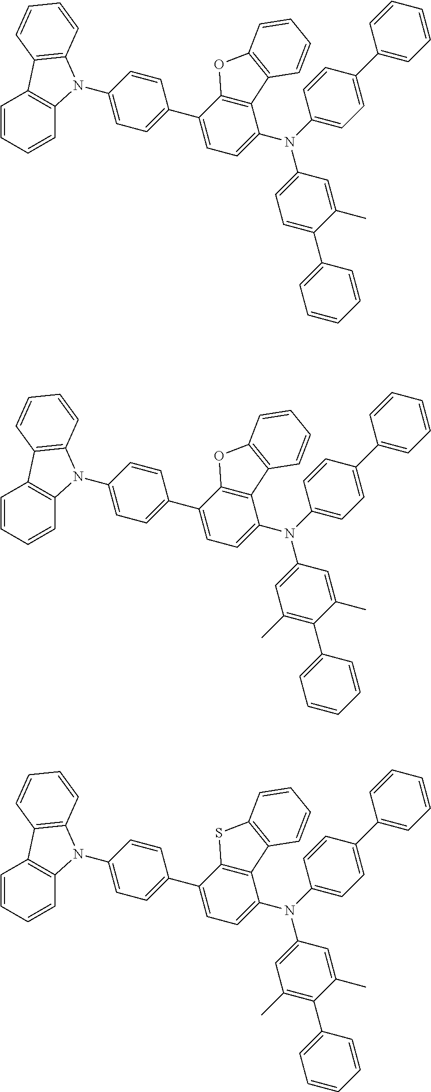 [SMILES: Cc1cc(N(c2ccc(-c3ccccc3)cc2)c2ccc(-c3ccc(-n4c5ccccc5c5ccccc54)cc3)c3oc4ccccc4c23)ccc1-c1ccccc1 ; Cc1cc(N(c2ccc(-c3ccccc3)cc2)c2ccc(-c3ccc(-n4c5ccccc5c5ccccc54)cc3)c3oc4ccccc4c23)cc(C)c1-c1ccccc1 ; Cc1cc(N(c2ccc(-c3ccccc3)cc2)c2ccc(-c3ccc(-n4c5ccccc5c5ccccc54)cc3)c3sc4ccccc4c23)cc(C)c1-c1ccccc1]

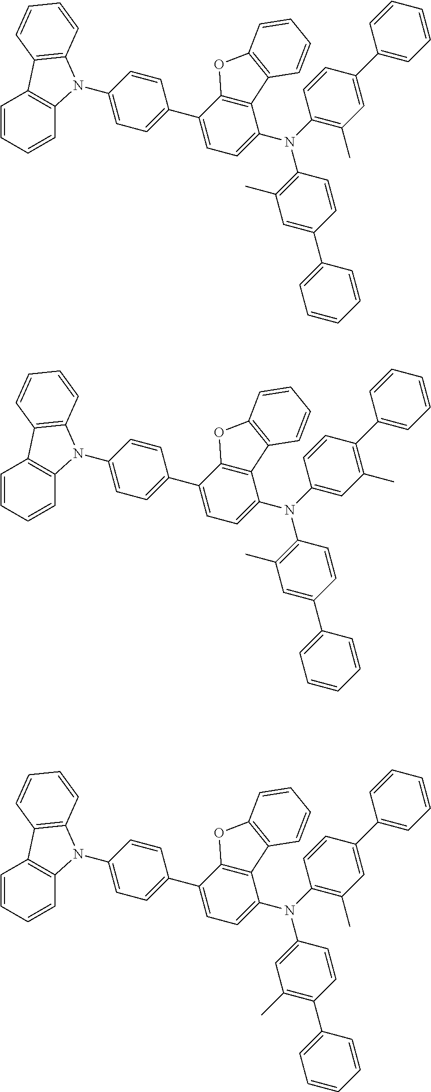 [SMILES: Cc1cc(-c2ccccc2)ccc1N(c1ccc(-c2ccccc2)cc1C)c1ccc(-c2ccc(-n3c4ccccc4c4ccccc43)cc2)c2oc3ccccc3c12 ; Cc1cc(N(c2ccc(-c3ccccc3)cc2C)c2ccc(-c3ccc(-n4c5ccccc5c5ccccc54)cc3)c3oc4ccccc4c23)ccc1-c1ccccc1 ; Cc1cc(N(c2ccc(-c3ccccc3)cc2C)c2ccc(-c3ccc(-n4c5ccccc5c5ccccc54)cc3)c3oc4ccccc4c23)ccc1-c1ccccc1]

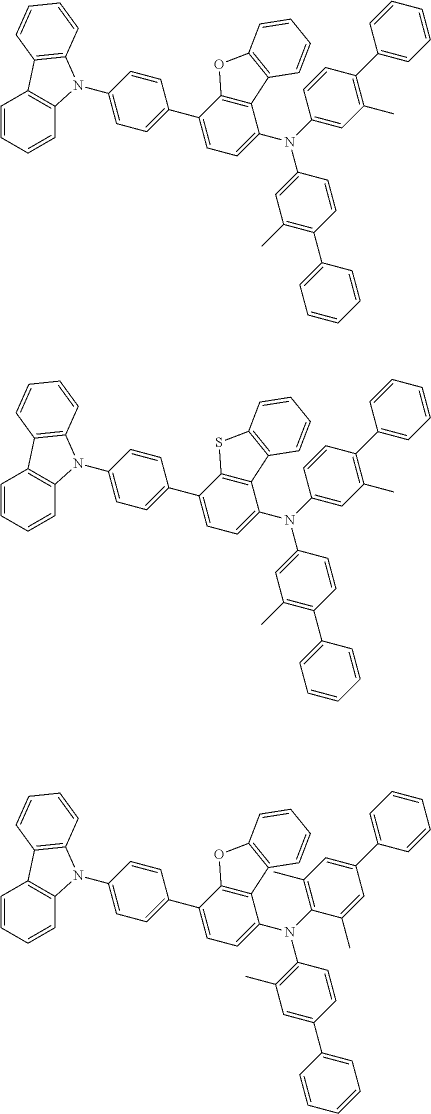 [SMILES: Cc1cc(N(c2ccc(-c3ccccc3)c(C)c2)c2ccc(-c3ccc(-n4c5ccccc5c5ccccc54)cc3)c3oc4ccccc4c23)ccc1-c1ccccc1 ; Cc1cc(N(c2ccc(-c3ccccc3)c(C)c2)c2ccc(-c3ccc(-n4c5ccccc5c5ccccc54)cc3)c3sc4ccccc4c23)ccc1-c1ccccc1 ; Cc1cc(-c2ccccc2)ccc1N(c1c(C)cc(-c2ccccc2)cc1C)c1ccc(-c2ccc(-n3c4ccccc4c4ccccc43)cc2)c2oc3ccccc3c12]

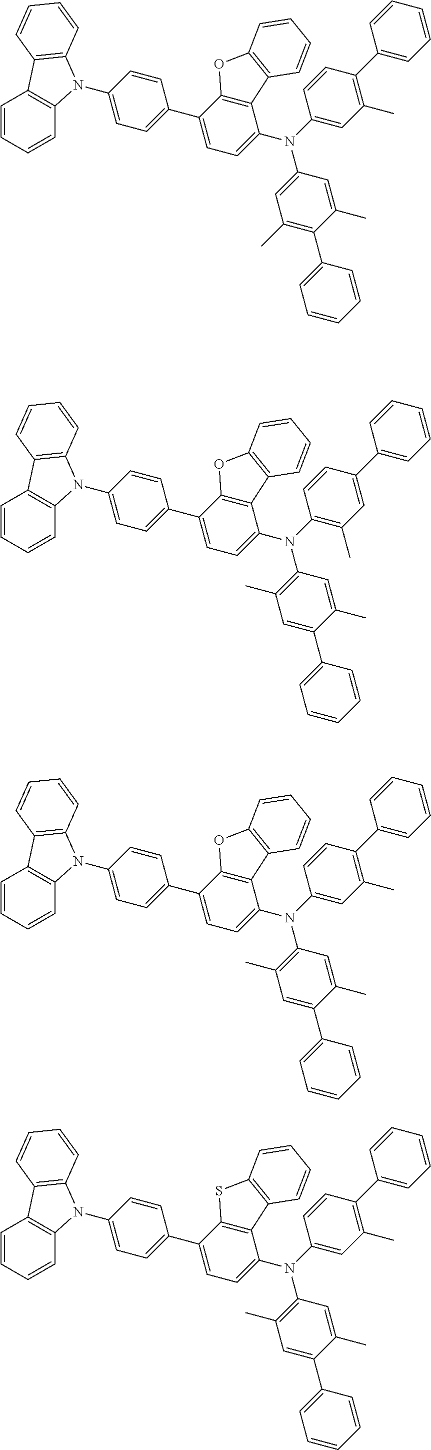 [SMILES: Cc1cc(N(c2cc(C)c(-c3ccccc3)c(C)c2)c2ccc(-c3ccc(-n4c5ccccc5c5ccccc54)cc3)c3oc4ccccc4c23)ccc1-c1ccccc1 ; Cc1cc(N(c2ccc(-c3ccccc3)cc2C)c2ccc(-c3ccc(-n4c5ccccc5c5ccccc54)cc3)c3oc4ccccc4c23)c(C)cc1-c1ccccc1 ; Cc1cc(N(c2cc(C)c(-c3ccccc3)cc2C)c2ccc(-c3ccc(-n4c5ccccc5c5ccccc54)cc3)c3oc4ccccc4c23)ccc1-c1ccccc1 ; Cc1cc(N(c2cc(C)c(-c3ccccc3)cc2C)c2ccc(-c3ccc(-n4c5ccccc5c5ccccc54)cc3)c3sc4ccccc4c23)ccc1-c1ccccc1]

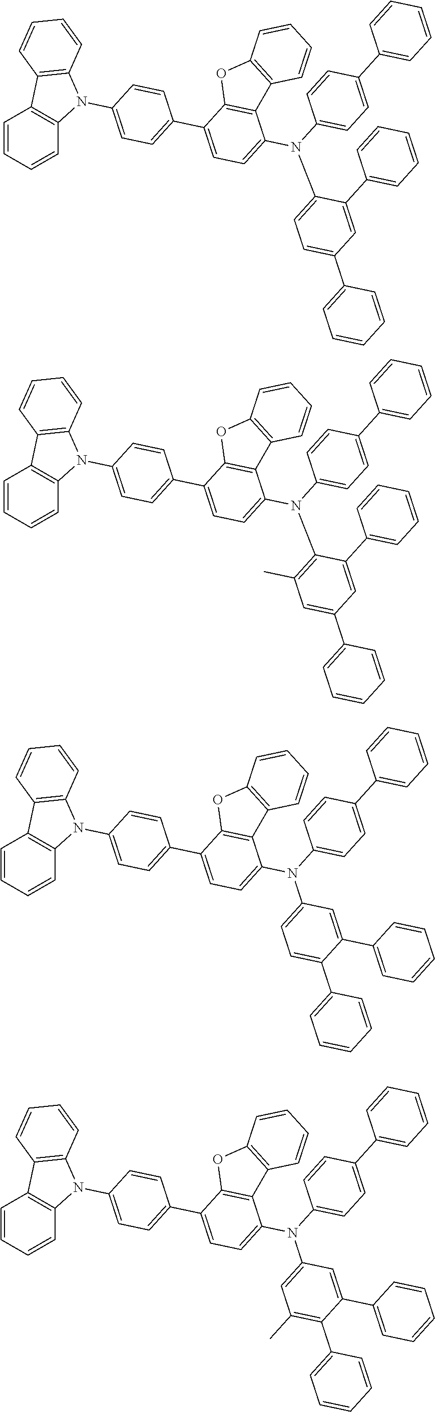

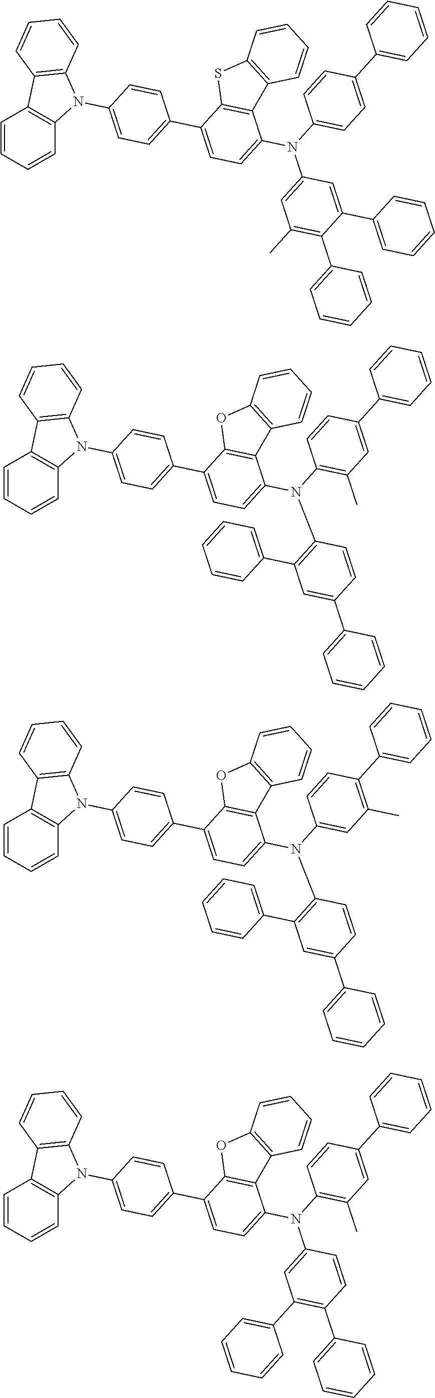

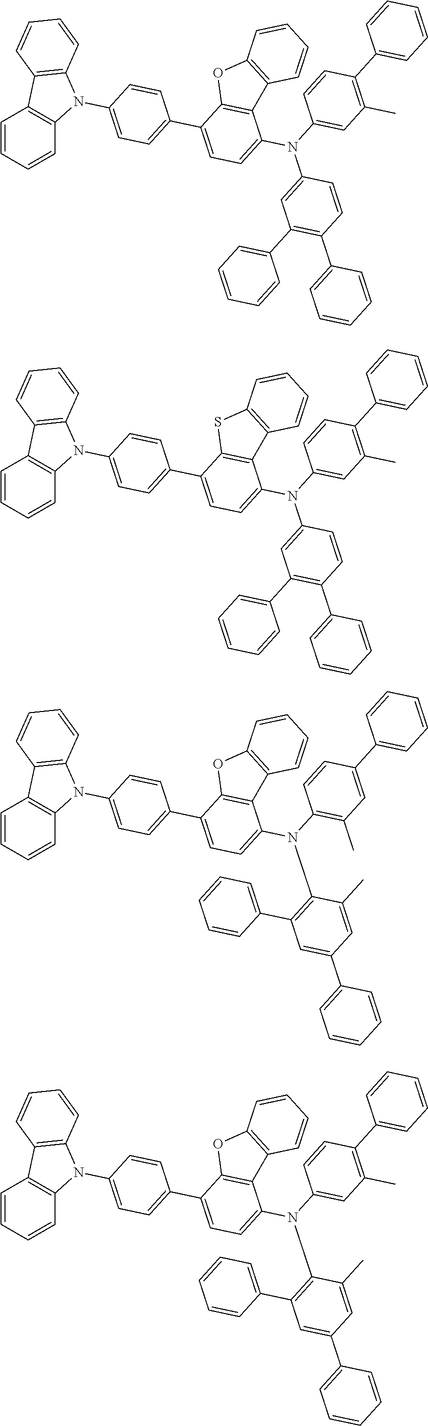

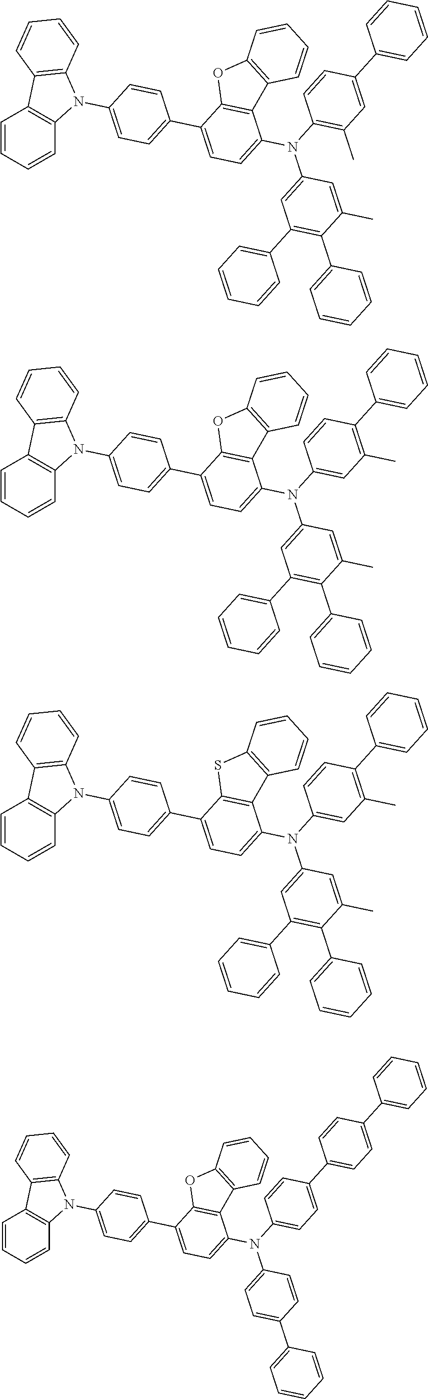

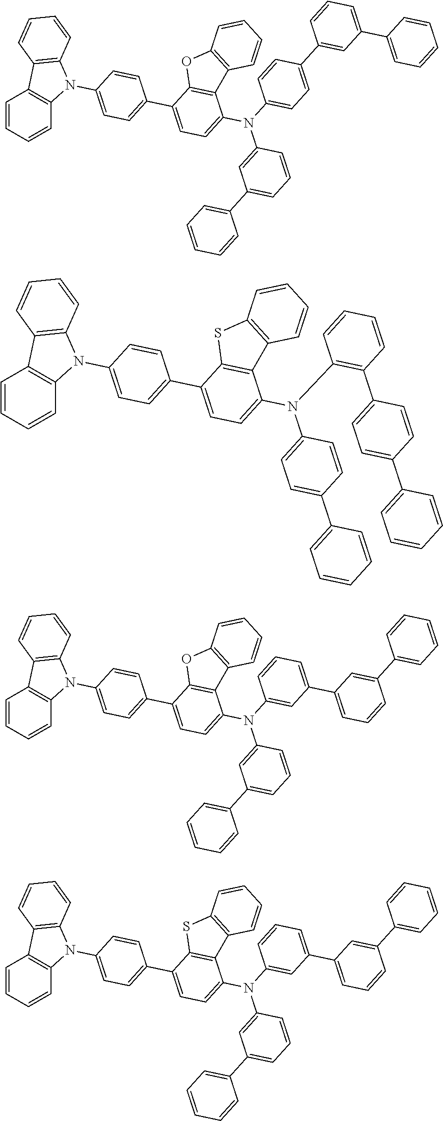 [SMILES: c1ccc(-c2cccc(-c3ccc(N(c4cccc(-c5ccccc5)c4)c4ccc(-c5ccc(-n6c7ccccc7c7ccccc76)cc5)c5oc6ccccc6c45)cc3)c2)cc1 ; c1ccc(-c2ccc(-c3ccccc3N(c3ccc(-c4ccccc4)cc3)c3ccc(-c4ccc(-n5c6ccccc6c6ccccc65)cc4)c4sc5ccccc5c34)cc2)cc1 ; c1ccc(-c2cccc(-c3cccc(N(c4cccc(-c5ccccc5)c4)c4ccc(-c5ccc(-n6c7ccccc7c7ccccc76)cc5)c5oc6ccccc6c45)c3)c2)cc1 ; c1ccc(-c2cccc(-c3cccc(N(c4cccc(-c5ccccc5)c4)c4ccc(-c5ccc(-n6c7ccccc7c7ccccc76)cc5)c5sc6ccccc6c45)c3)c2)cc1]

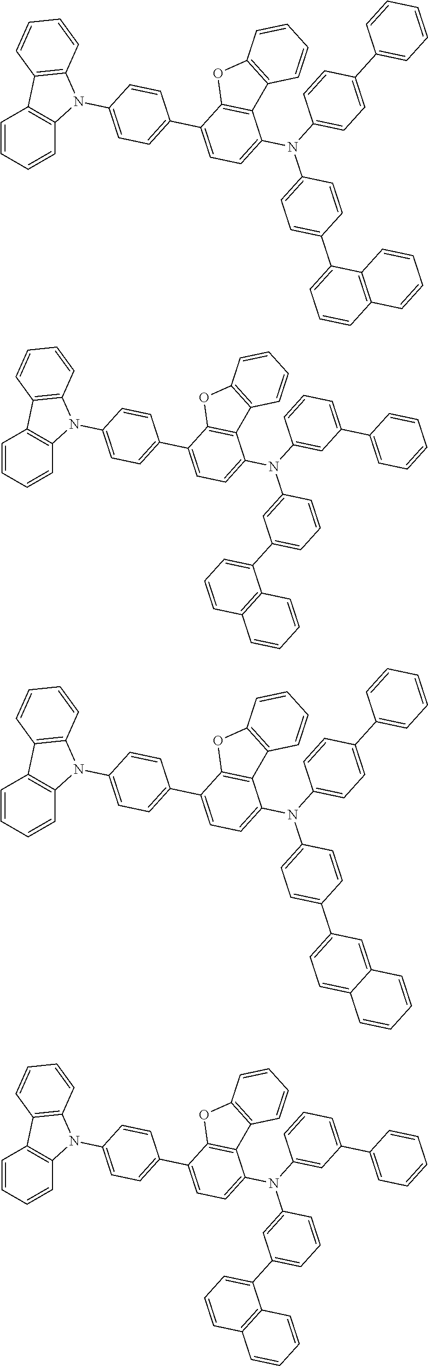 [SMILES: c1ccc(-c2ccc(N(c3ccc(-c4cccc5ccccc45)cc3)c3ccc(-c4ccc(-n5c6ccccc6c6ccccc65)cc4)c4oc5ccccc5c34)cc2)cc1 ; c1ccc(-c2cccc(N(c3cccc(-c4cccc5ccccc45)c3)c3ccc(-c4ccc(-n5c6ccccc6c6ccccc65)cc4)c4oc5ccccc5c34)c2)cc1 ; c1ccc(-c2ccc(N(c3ccc(-c4ccc5ccccc5c4)cc3)c3ccc(-c4ccc(-n5c6ccccc6c6ccccc65)cc4)c4oc5ccccc5c34)cc2)cc1 ; c1ccc(-c2cccc(N(c3cccc(-c4cccc5ccccc45)c3)c3ccc(-c4ccc(-n5c6ccccc6c6ccccc65)cc4)c4oc5ccccc5c34)c2)cc1]

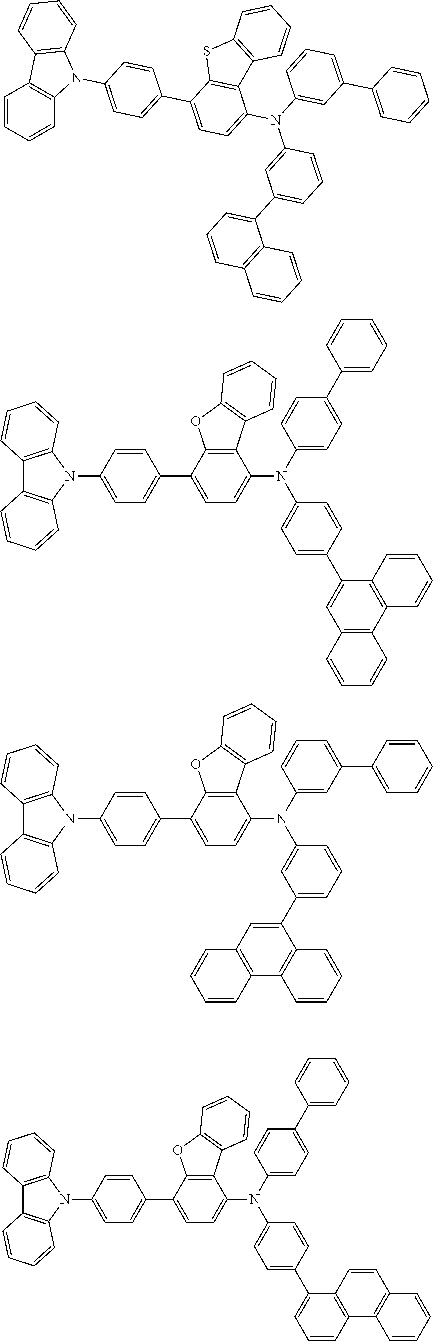 [SMILES: c1ccc(-c2cccc(N(c3cccc(-c4cccc5ccccc45)c3)c3ccc(-c4ccc(-n5c6ccccc6c6ccccc65)cc4)c4sc5ccccc5c34)c2)cc1 ; c1ccc(-c2ccc(N(c3ccc(-c4cc5ccccc5c5ccccc45)cc3)c3ccc(-c4ccc(-n5c6ccccc6c6ccccc65)cc4)c4oc5ccccc5c34)cc2)cc1 ; c1ccc(-c2cccc(N(c3cccc(-c4cc5ccccc5c5ccccc45)c3)c3ccc(-c4ccc(-n5c6ccccc6c6ccccc65)cc4)c4oc5ccccc5c34)c2)cc1 ; c1ccc(-c2ccc(N(c3ccc(-c4cccc5c4ccc4ccccc45)cc3)c3ccc(-c4ccc(-n5c6ccccc6c6ccccc65)cc4)c4oc5ccccc5c34)cc2)cc1]

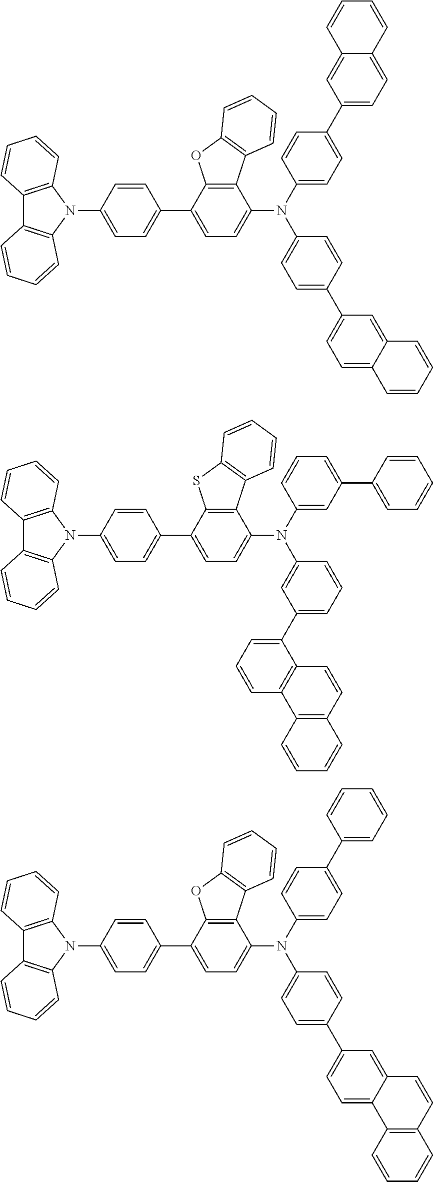

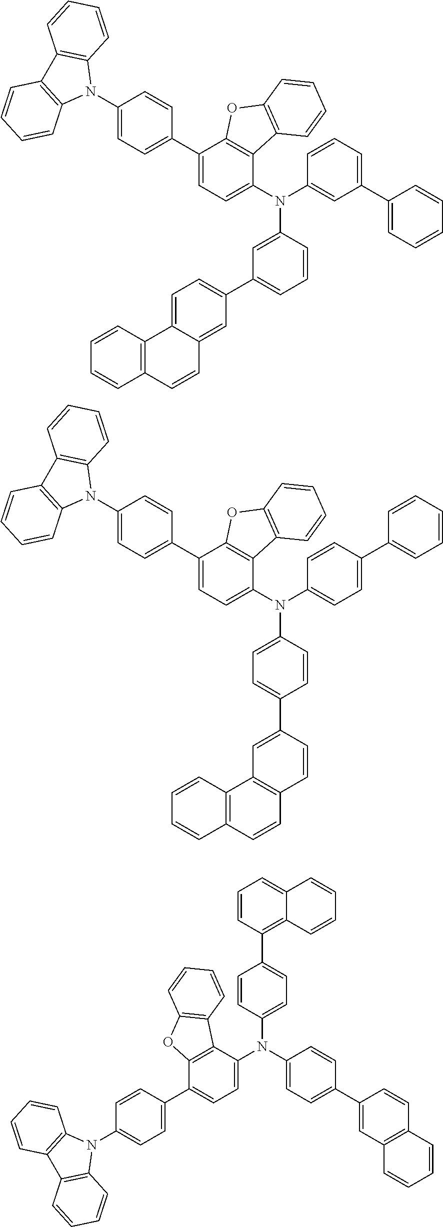

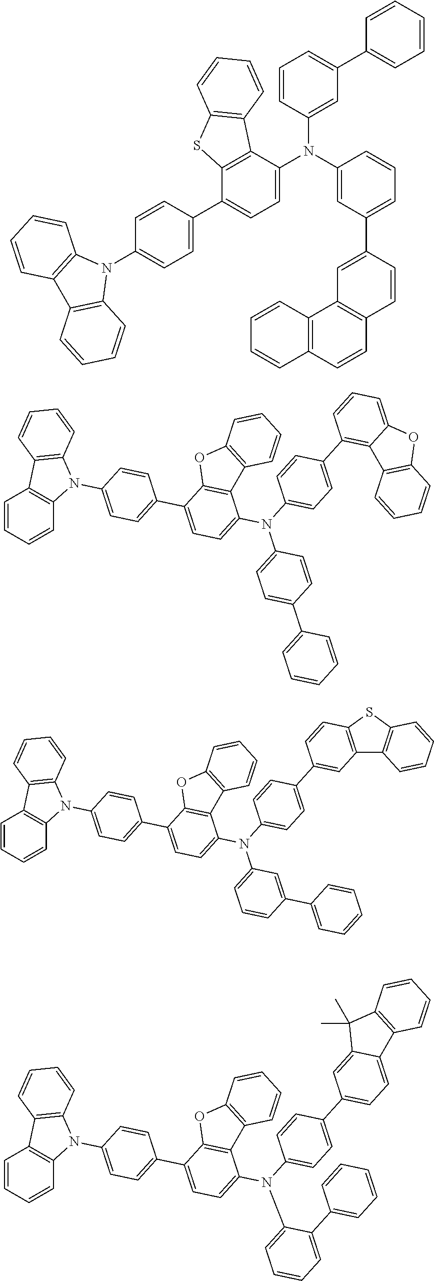

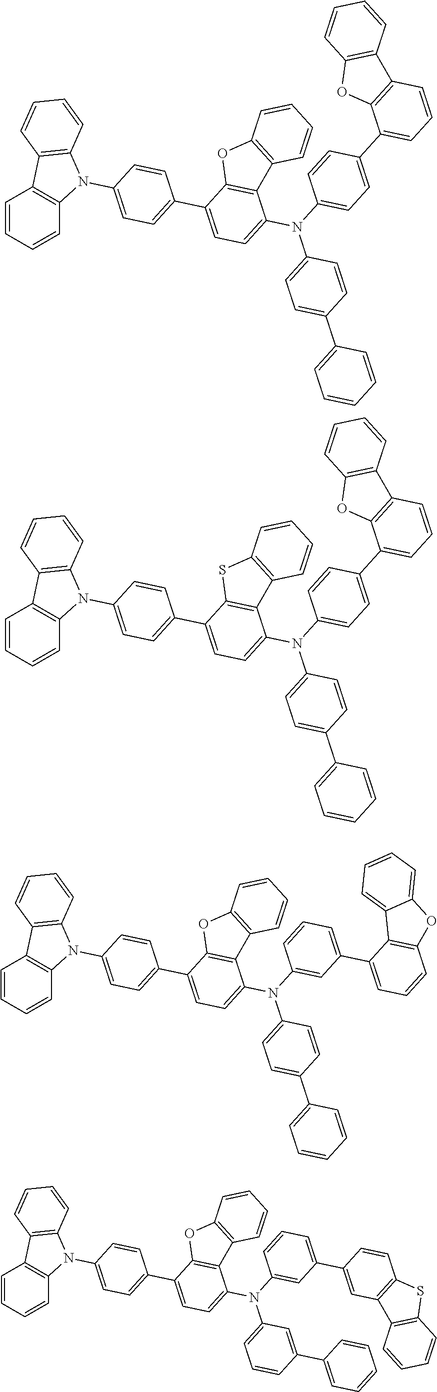

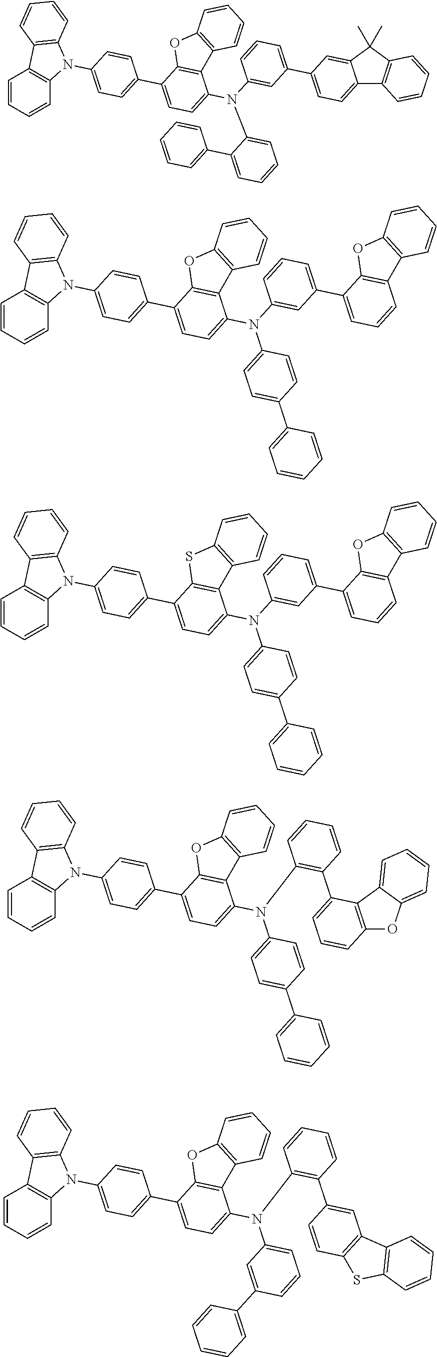

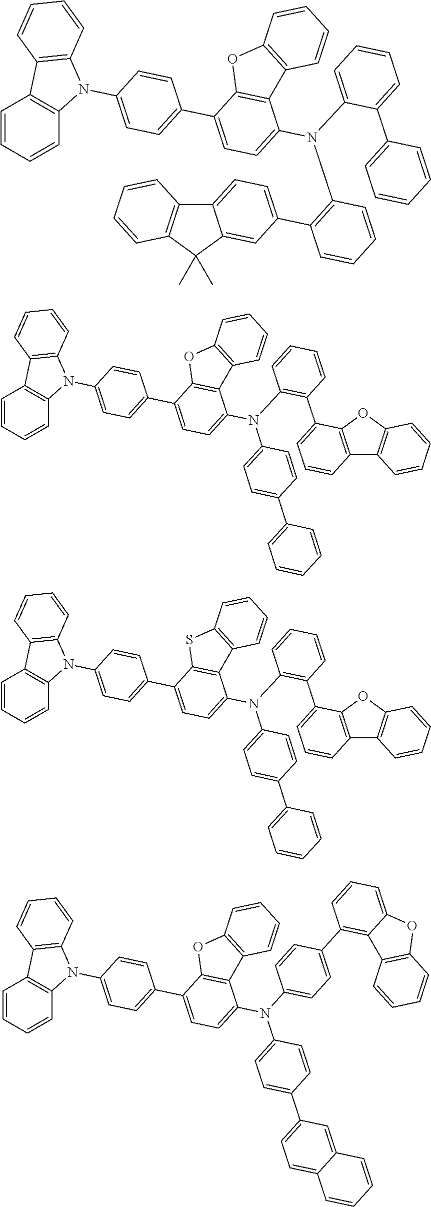

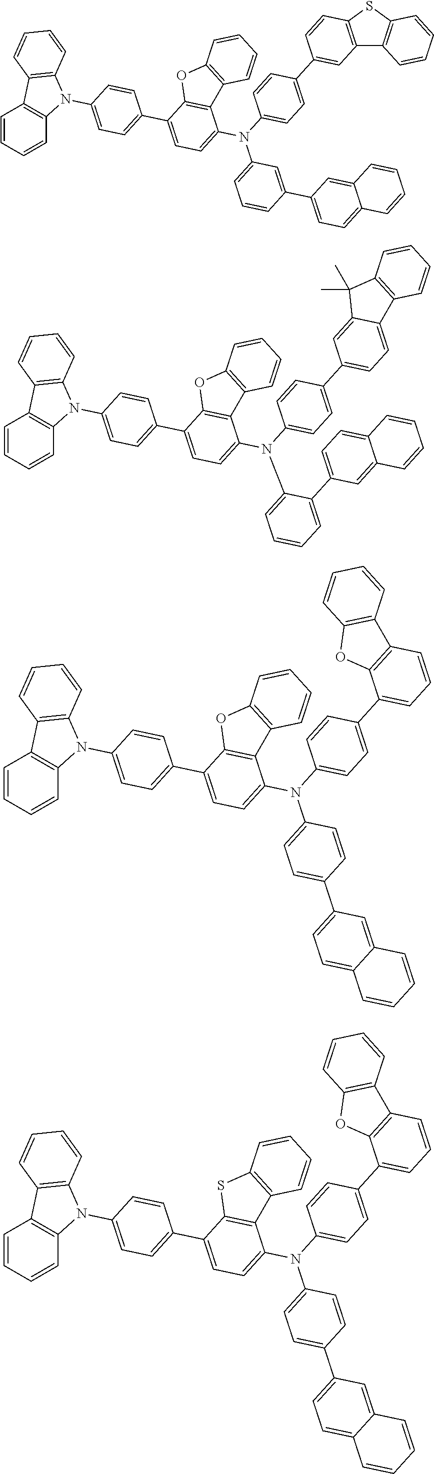

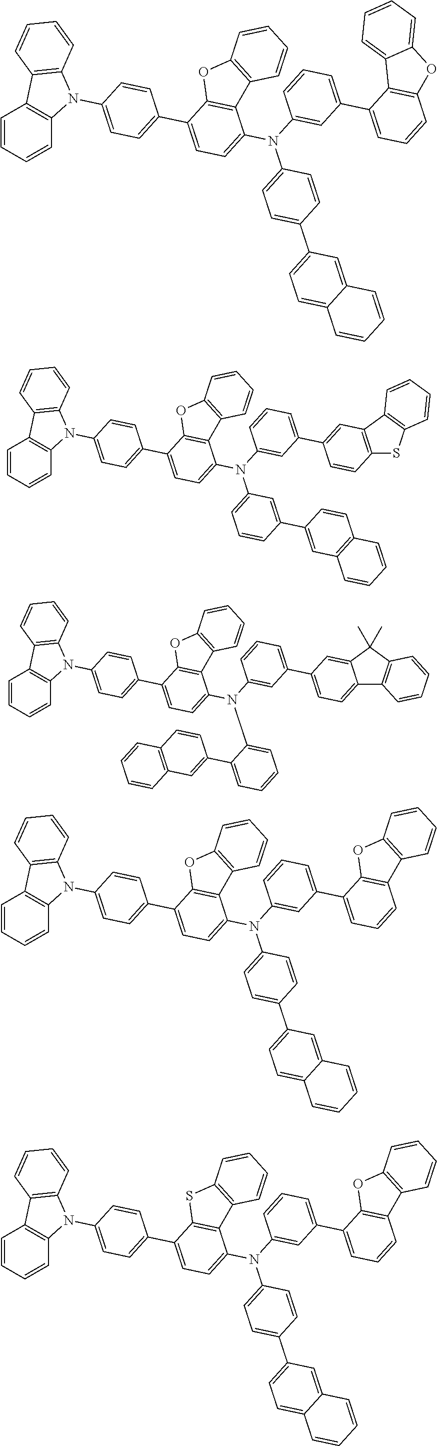

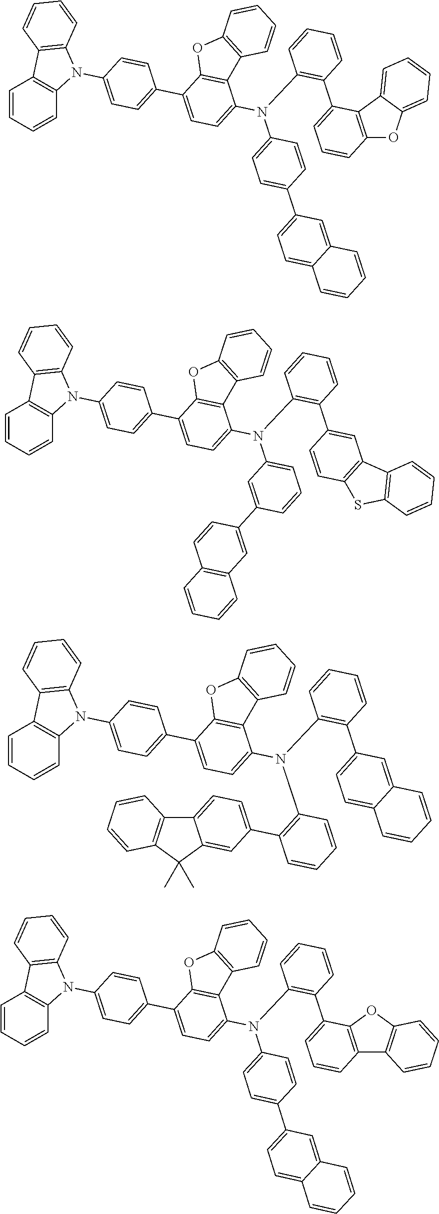 [SMILES: c1ccc(N(c2ccc(-c3ccc4ccccc4c3)cc2)c2ccc(-c3ccc(-n4c5ccccc5c5ccccc54)cc3)c3oc4ccccc4c23)c(-c2cccc3oc4ccccc4c23)c1 ; c1cc(-c2ccc3ccccc3c2)cc(N(c2ccccc2-c2ccc3sc4ccccc4c3c2)c2ccc(-c3ccc(-n4c5ccccc5c5ccccc54)cc3)c3oc4ccccc4c23)c1 ; CC1(C)c2ccccc2-c2ccc(-c3ccccc3N(c3ccccc3-c3ccc4ccccc4c3)c3ccc(-c4ccc(-n5c6ccccc6c6ccccc65)cc4)c4oc5ccccc5c34)cc21 ; c1ccc(N(c2ccc(-c3ccc4ccccc4c3)cc2)c2ccc(-c3ccc(-n4c5ccccc5c5ccccc54)cc3)c3oc4ccccc4c23)c(-c2cccc3c2oc2ccccc23)c1]

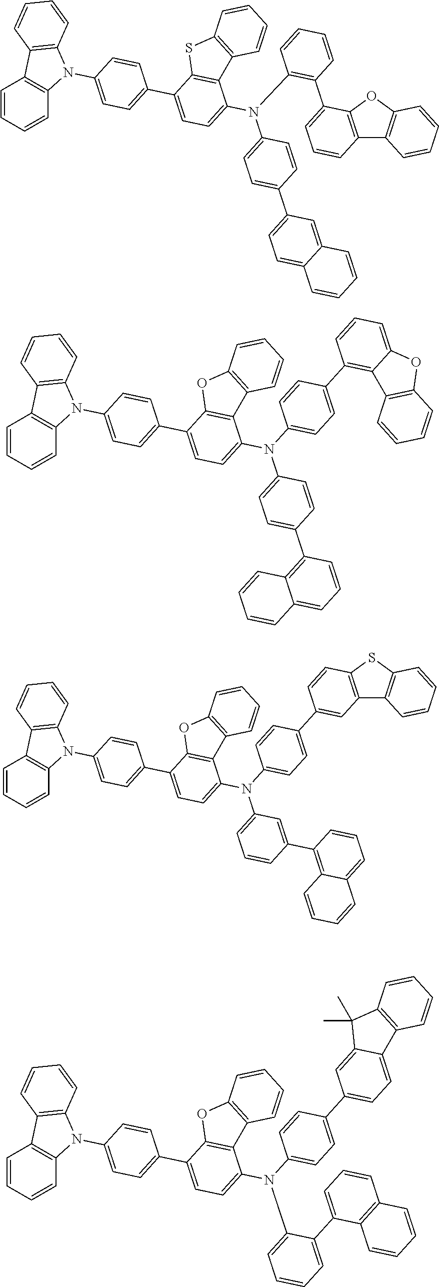

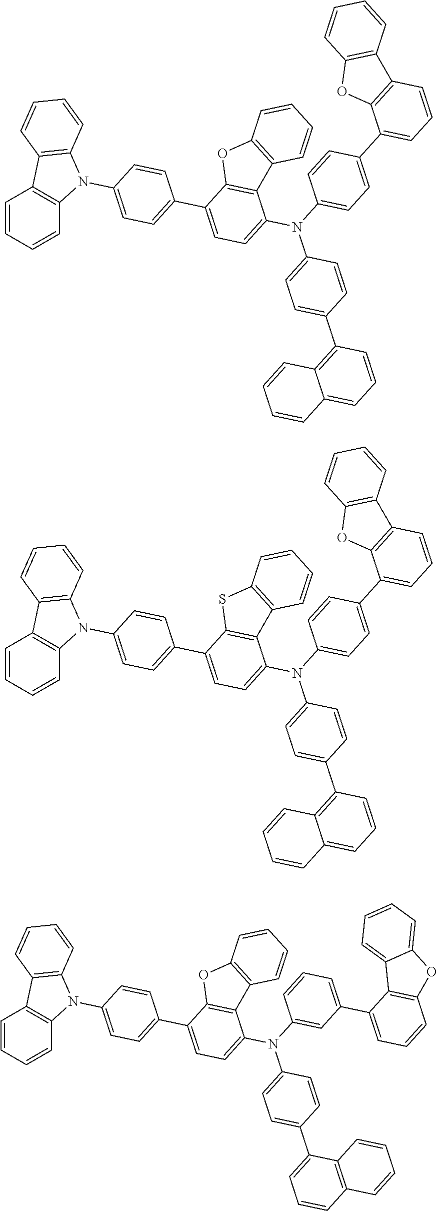

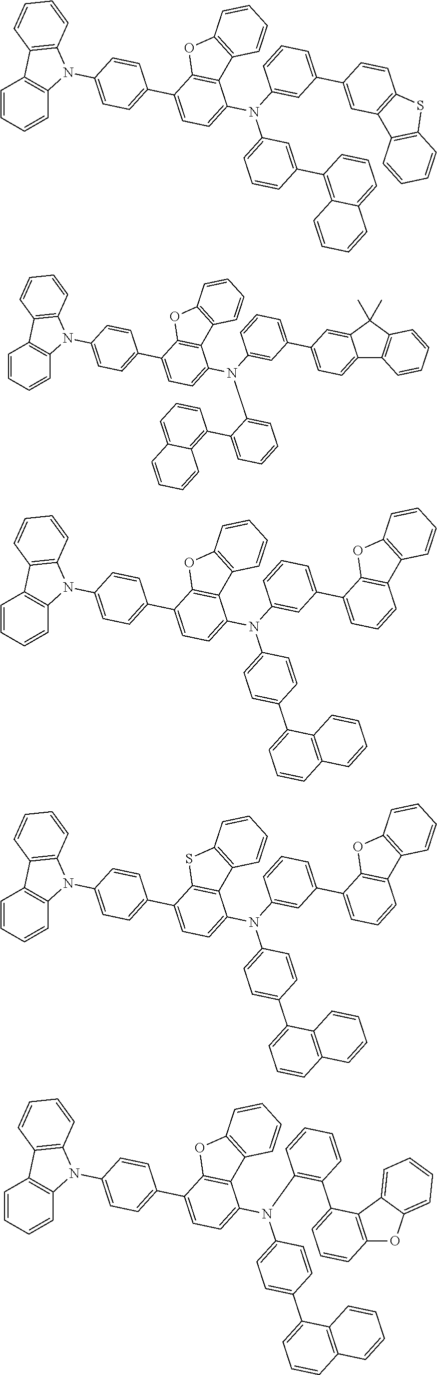

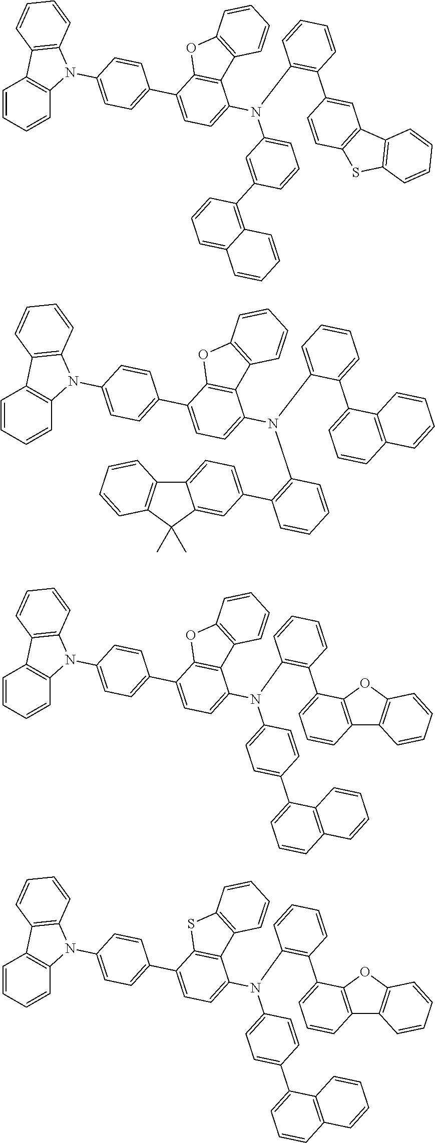

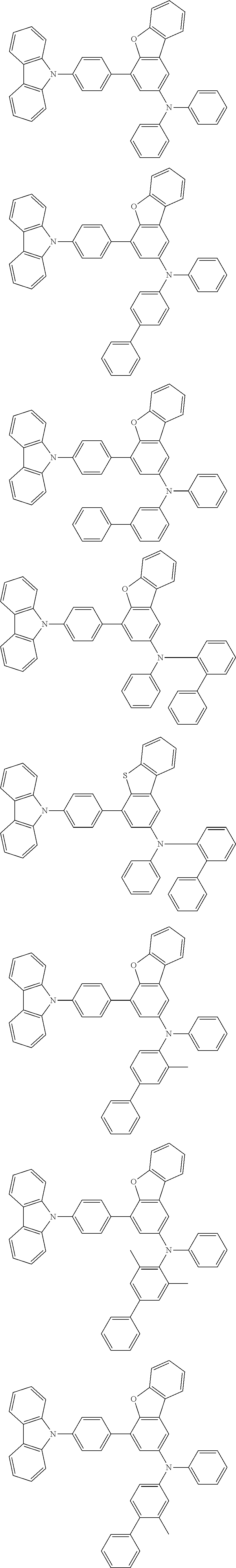

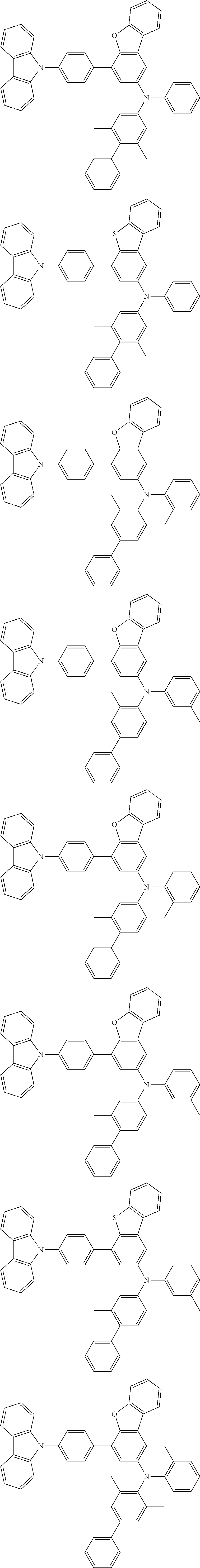

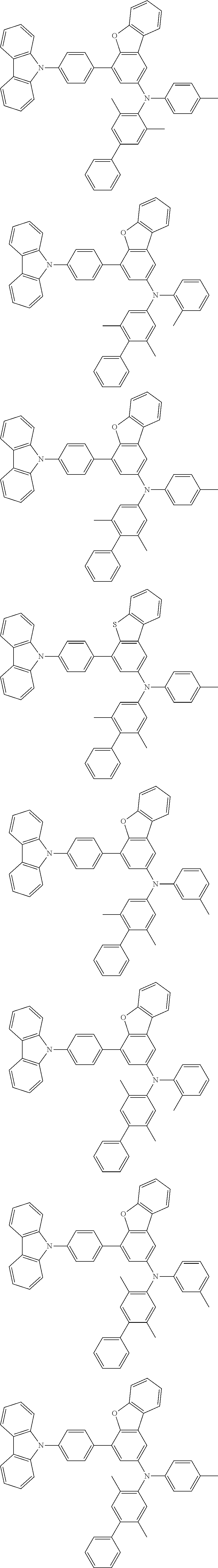

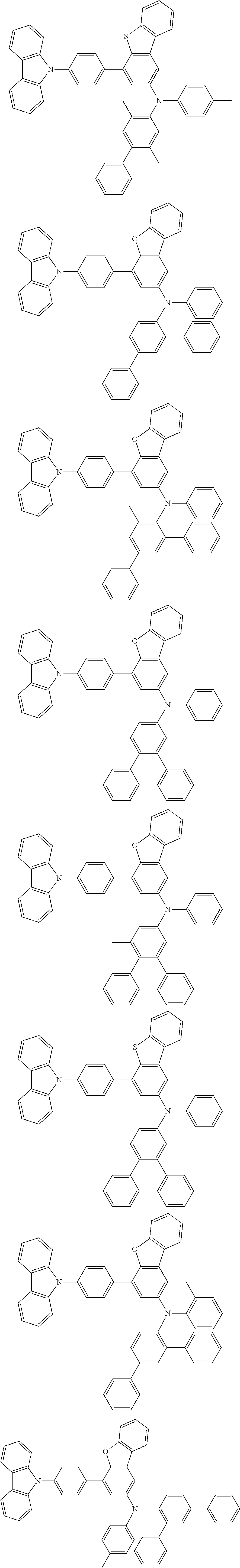

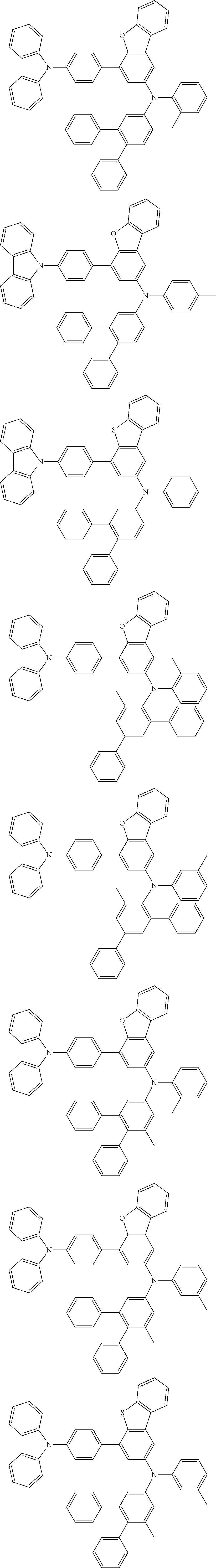

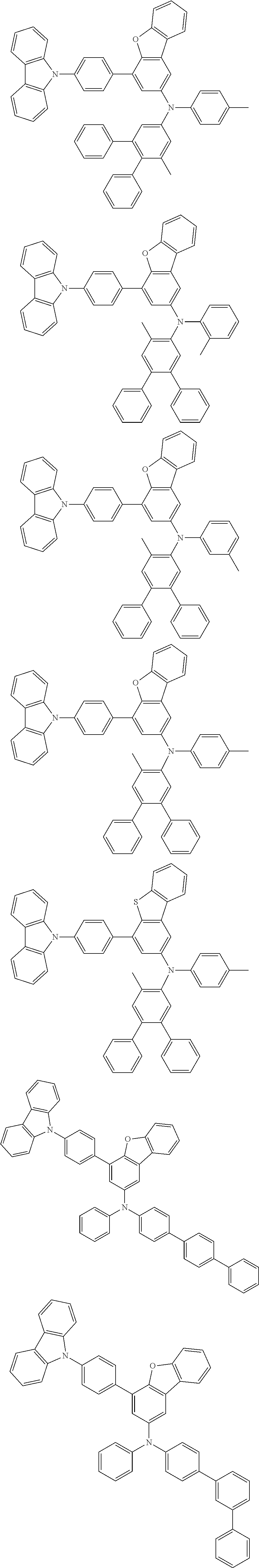

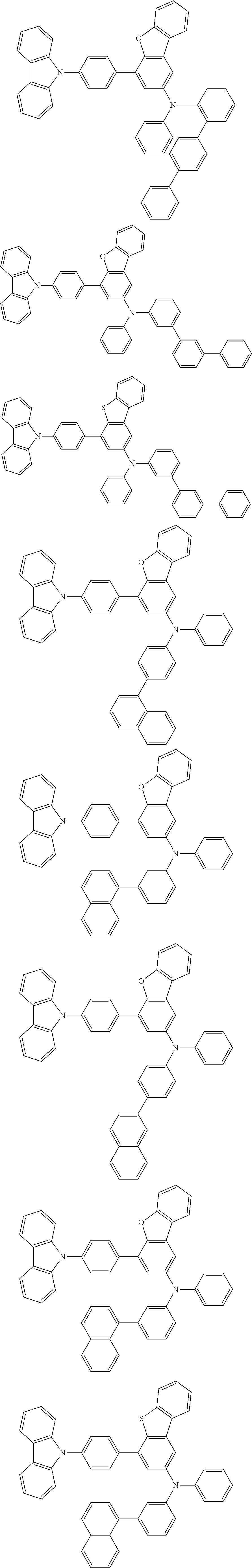

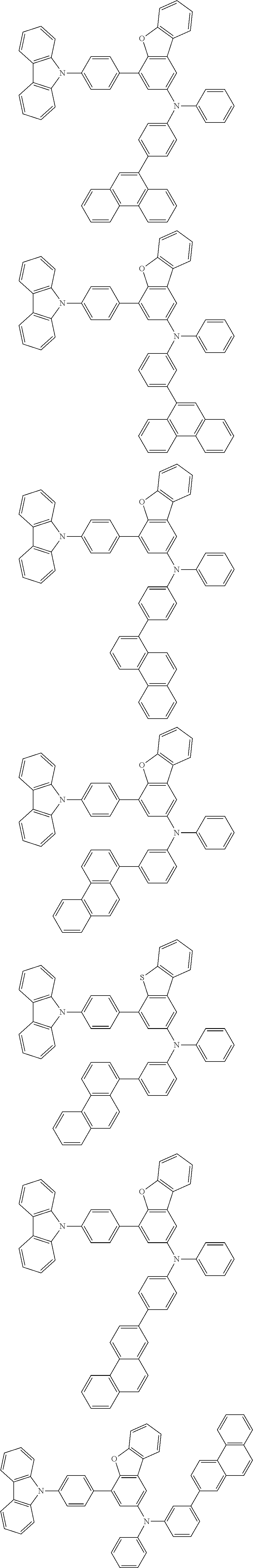

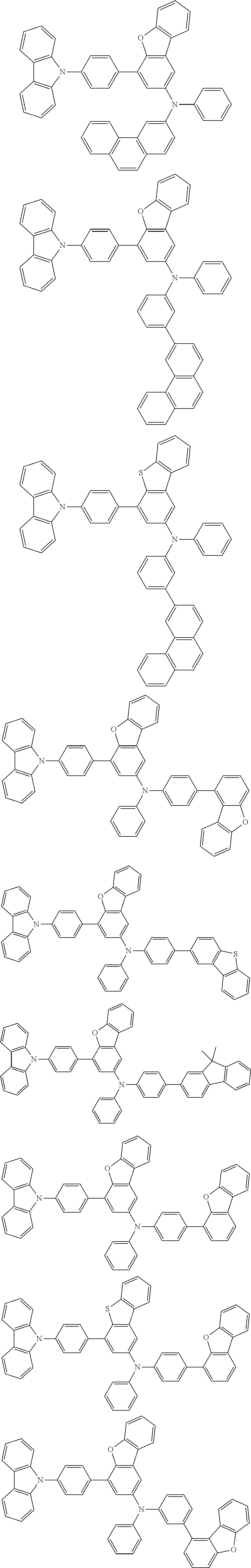

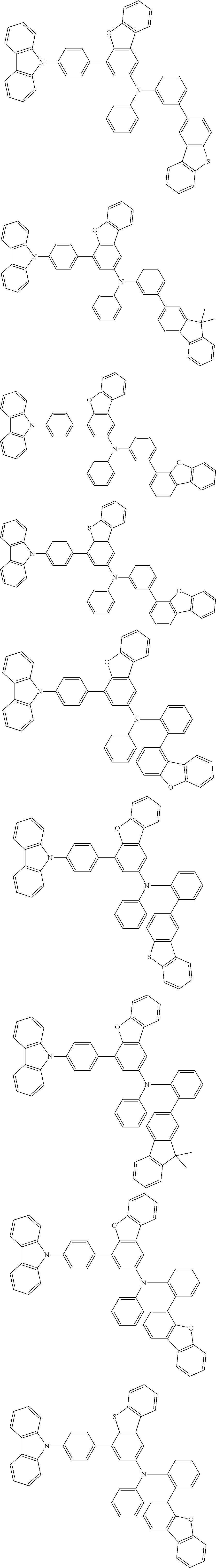

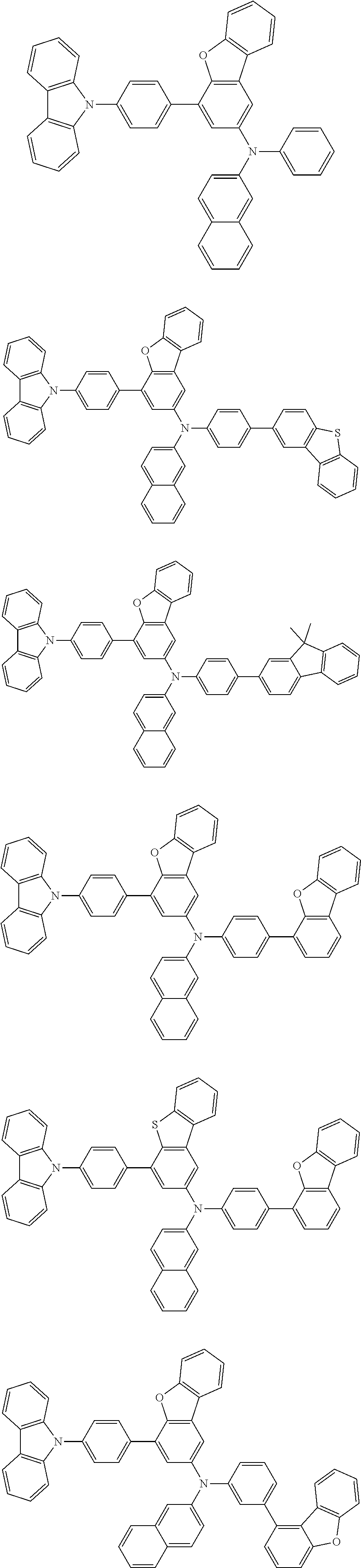

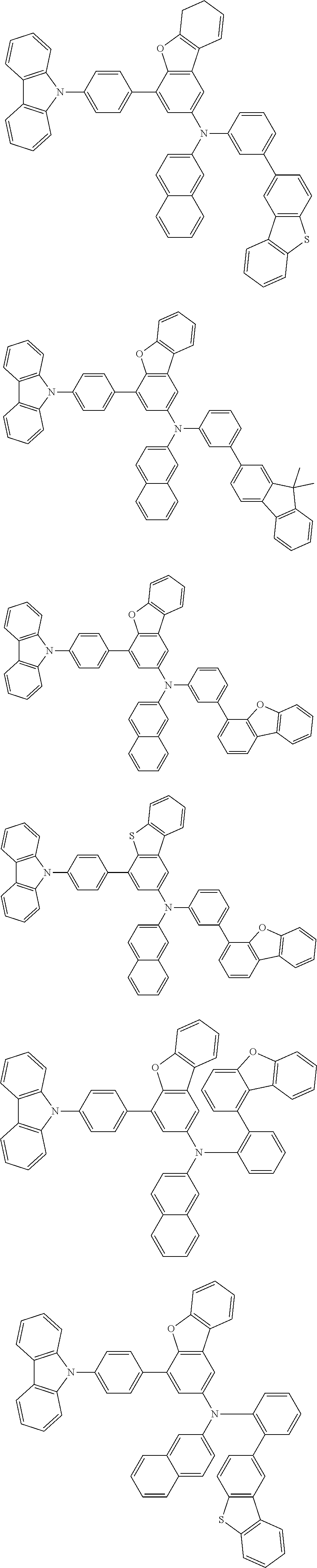

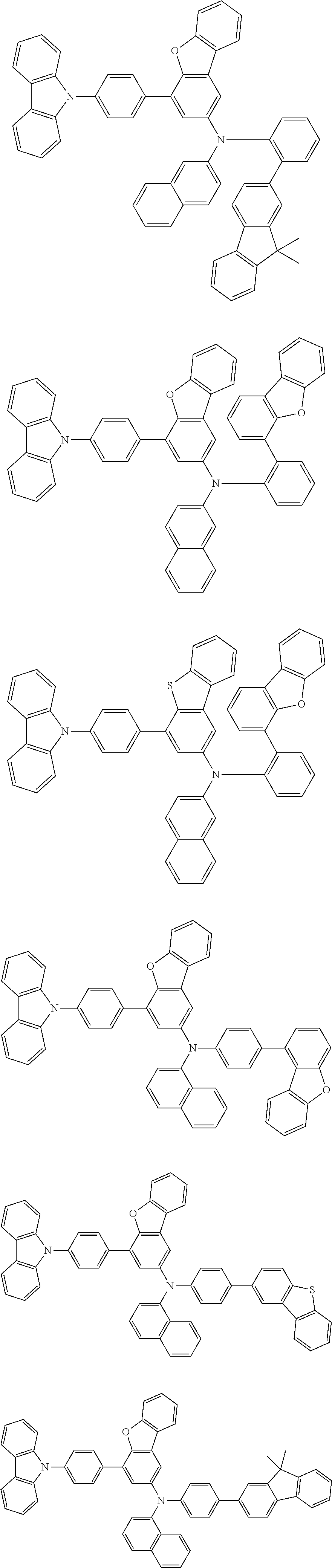

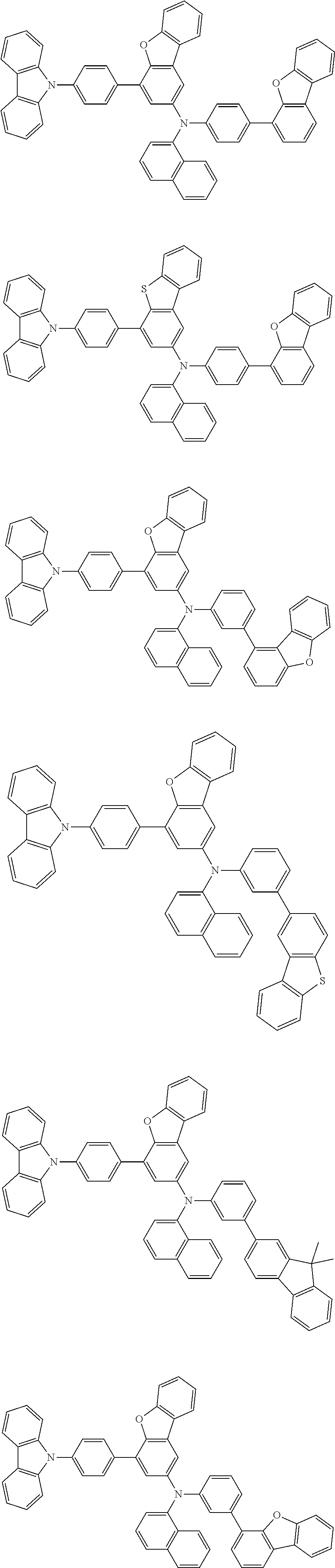

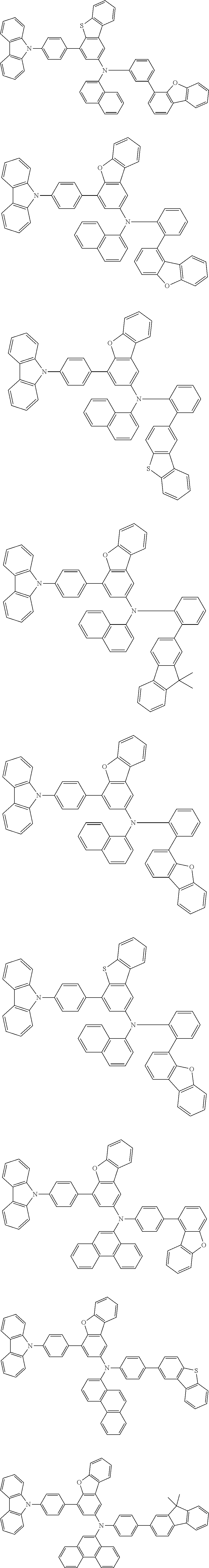

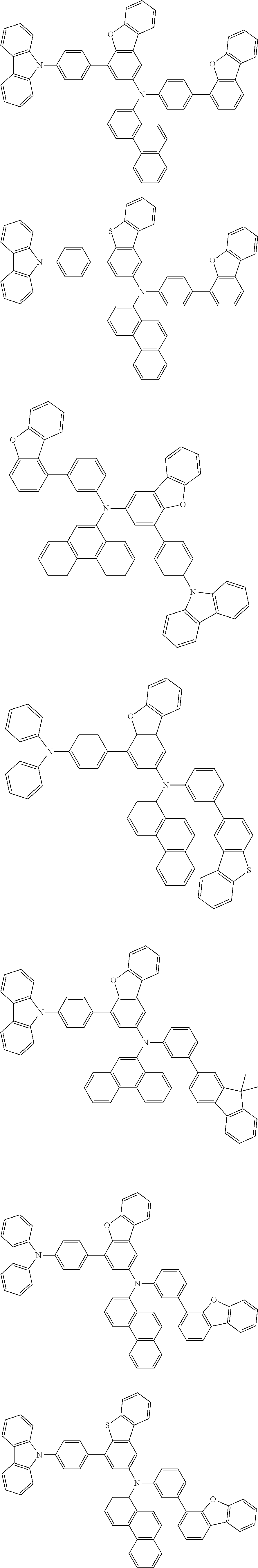

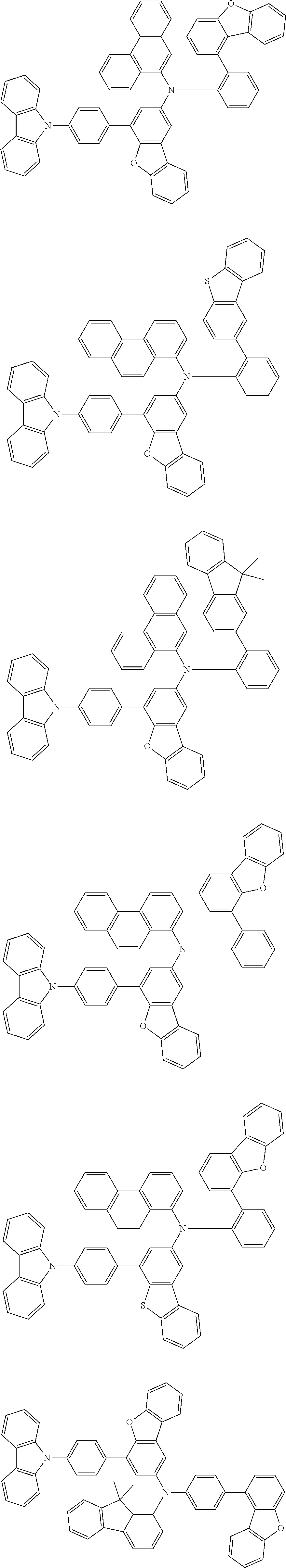

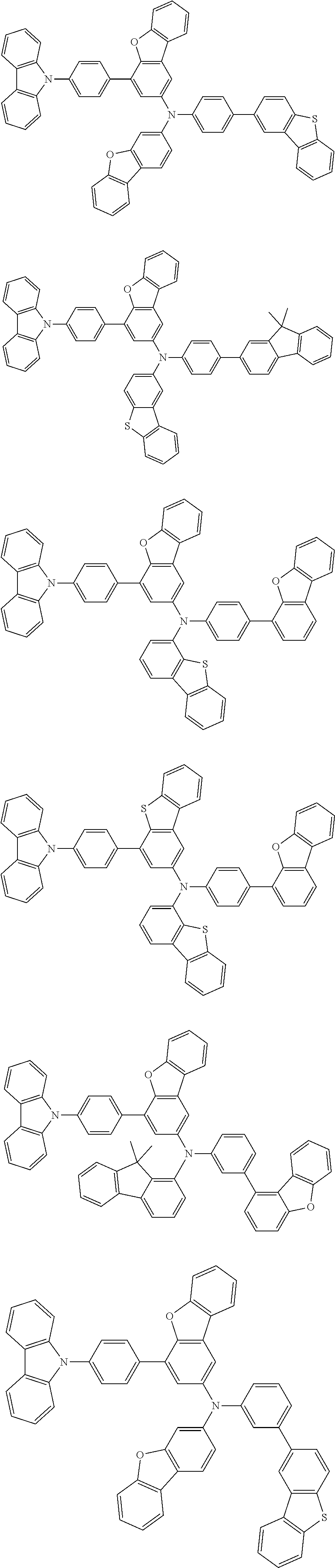

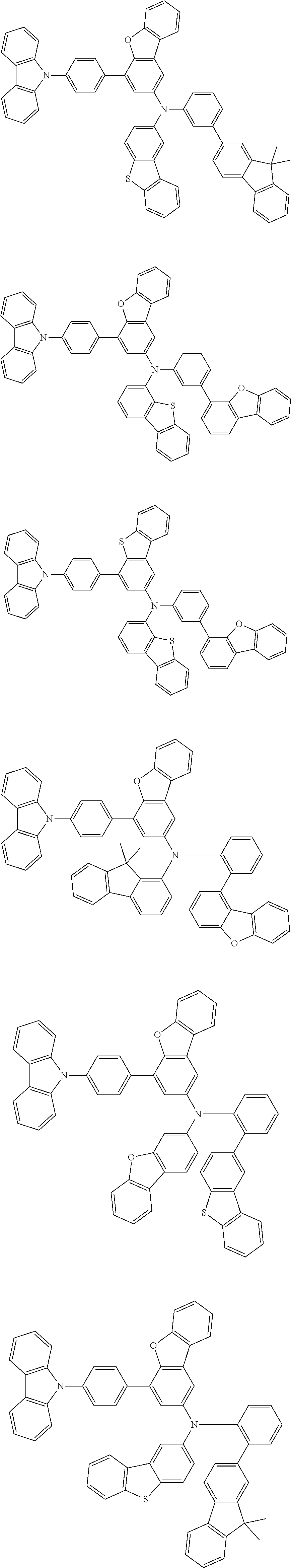

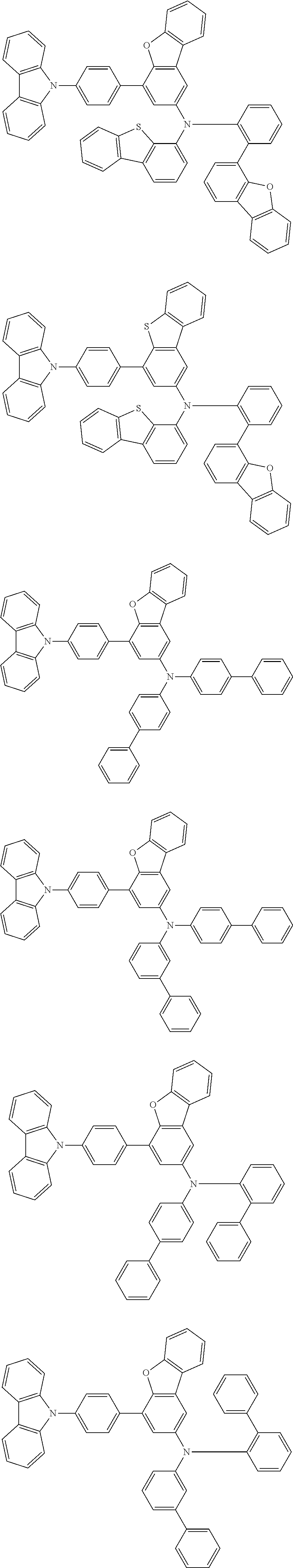

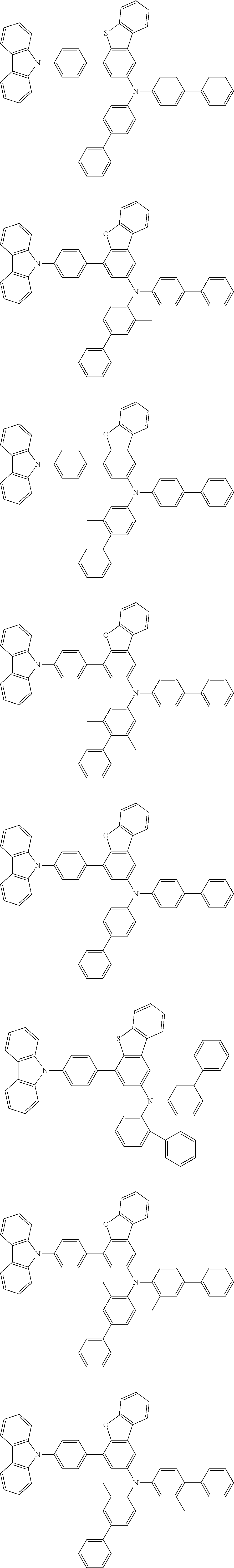

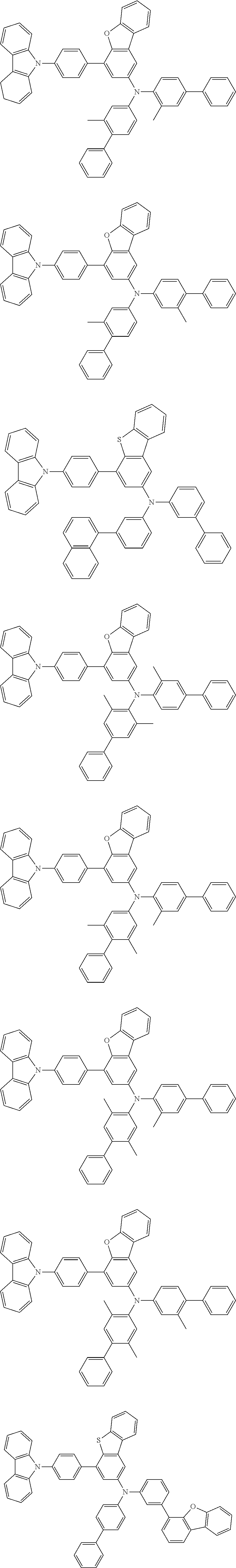

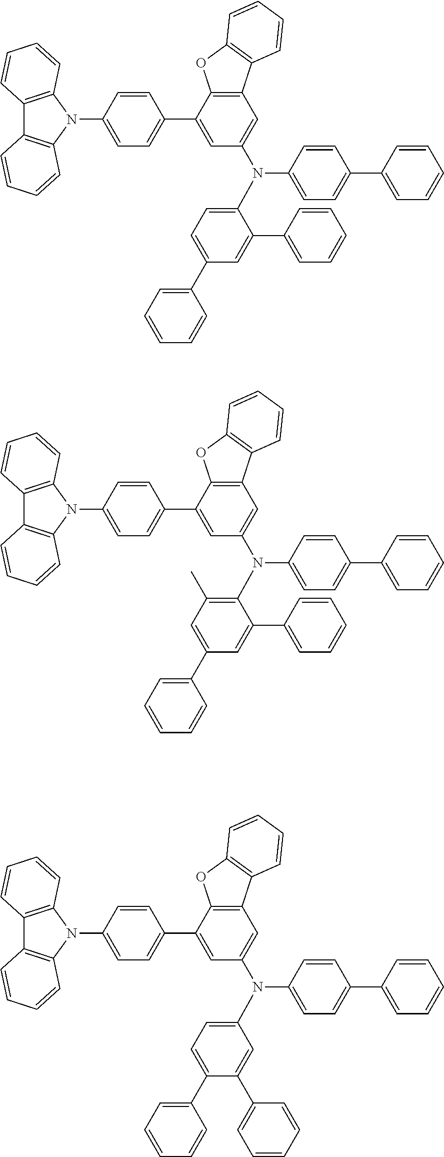 [SMILES: c1ccc(-c2ccc(N(c3cc(-c4ccc(-n5c6ccccc6c6ccccc65)cc4)c4oc5ccccc5c4c3)c3ccc(-c4ccccc4)cc3-c3ccccc3)cc2)cc1 ; Cc1cc(-c2ccccc2)cc(-c2ccccc2)c1N(c1ccc(-c2ccccc2)cc1)c1cc(-c2ccc(-n3c4ccccc4c4ccccc43)cc2)c2oc3ccccc3c2c1 ; c1ccc(-c2ccc(N(c3ccc(-c4ccccc4)c(-c4ccccc4)c3)c3cc(-c4ccc(-n5c6ccccc6c6ccccc65)cc4)c4oc5ccccc5c4c3)cc2)cc1]

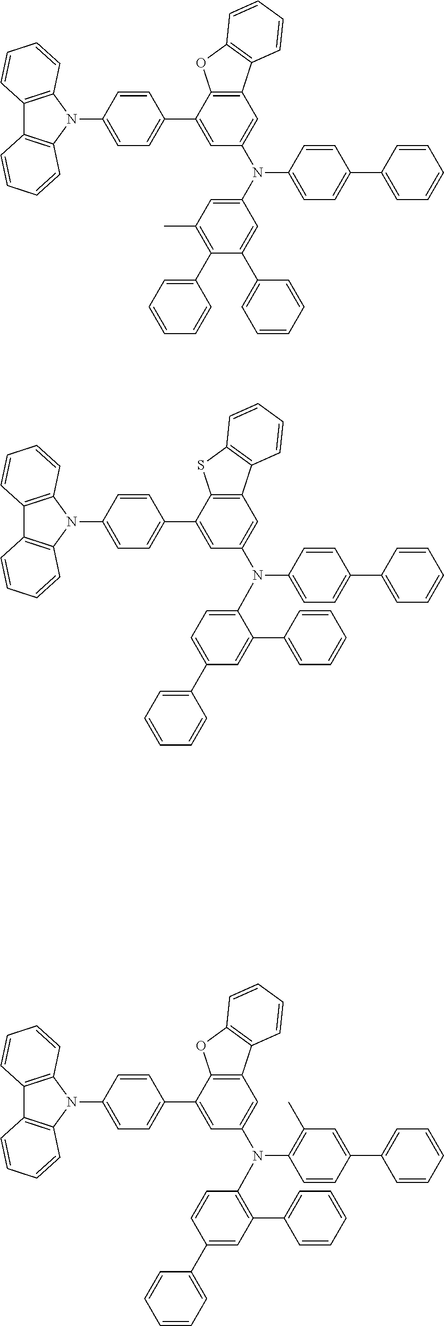 [SMILES: Cc1cc(N(c2ccc(-c3ccccc3)cc2)c2cc(-c3ccc(-n4c5ccccc5c5ccccc54)cc3)c3oc4ccccc4c3c2)cc(-c2ccccc2)c1-c1ccccc1 ; c1ccc(-c2ccc(N(c3cc(-c4ccc(-n5c6ccccc6c6ccccc65)cc4)c4sc5ccccc5c4c3)c3ccc(-c4ccccc4)cc3-c3ccccc3)cc2)cc1 ; Cc1cc(-c2ccccc2)ccc1N(c1cc(-c2ccc(-n3c4ccccc4c4ccccc43)cc2)c2oc3ccccc3c2c1)c1ccc(-c2ccccc2)cc1-c1ccccc1]

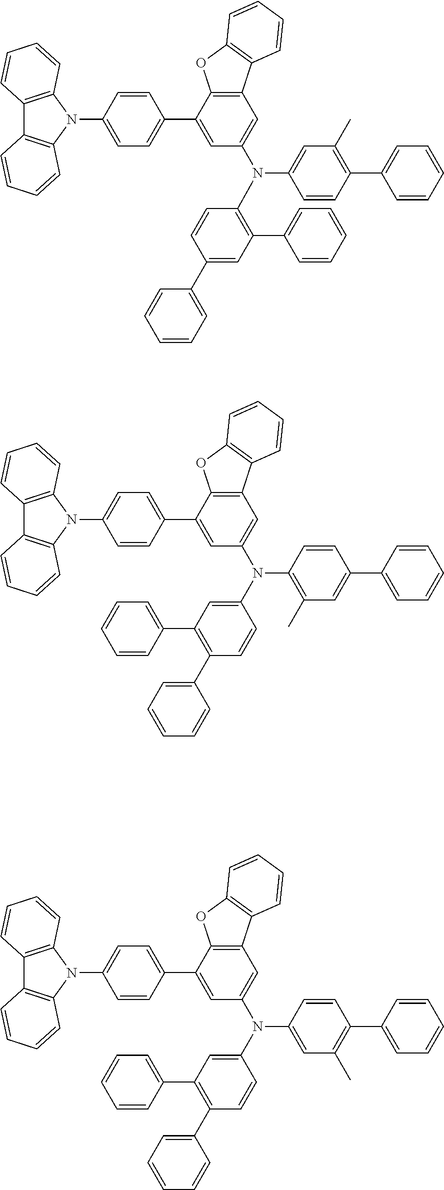 [SMILES: Cc1cc(N(c2cc(-c3ccc(-n4c5ccccc5c5ccccc54)cc3)c3oc4ccccc4c3c2)c2ccc(-c3ccccc3)cc2-c2ccccc2)ccc1-c1ccccc1 ; Cc1cc(-c2ccccc2)ccc1N(c1ccc(-c2ccccc2)c(-c2ccccc2)c1)c1cc(-c2ccc(-n3c4ccccc4c4ccccc43)cc2)c2oc3ccccc3c2c1 ; Cc1cc(N(c2ccc(-c3ccccc3)c(-c3ccccc3)c2)c2cc(-c3ccc(-n4c5ccccc5c5ccccc54)cc3)c3oc4ccccc4c3c2)ccc1-c1ccccc1]

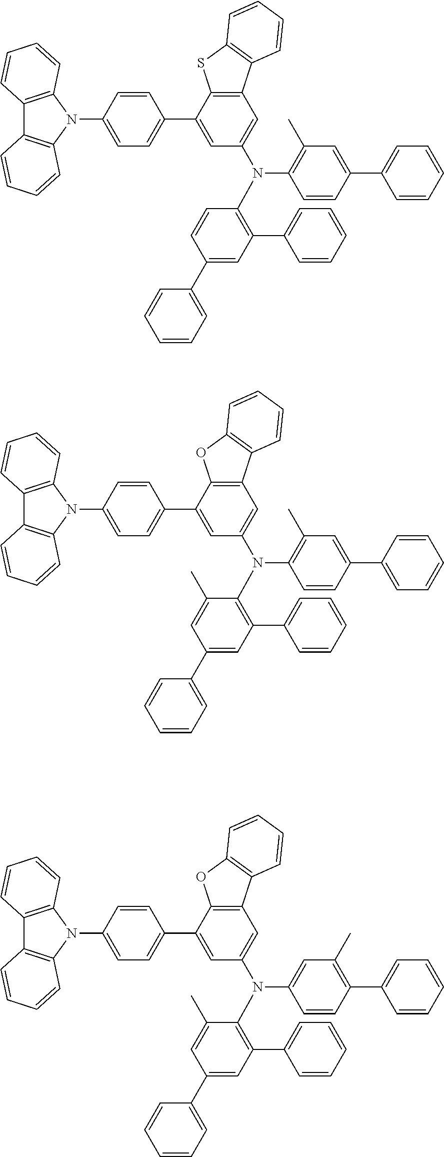 [SMILES: Cc1cc(-c2ccccc2)ccc1N(c1cc(-c2ccc(-n3c4ccccc4c4ccccc43)cc2)c2sc3ccccc3c2c1)c1ccc(-c2ccccc2)cc1-c1ccccc1 ; Cc1cc(-c2ccccc2)ccc1N(c1cc(-c2ccc(-n3c4ccccc4c4ccccc43)cc2)c2oc3ccccc3c2c1)c1c(C)cc(-c2ccccc2)cc1-c1ccccc1 ; Cc1cc(N(c2cc(-c3ccc(-n4c5ccccc5c5ccccc54)cc3)c3oc4ccccc4c3c2)c2c(C)cc(-c3ccccc3)cc2-c2ccccc2)ccc1-c1ccccc1]

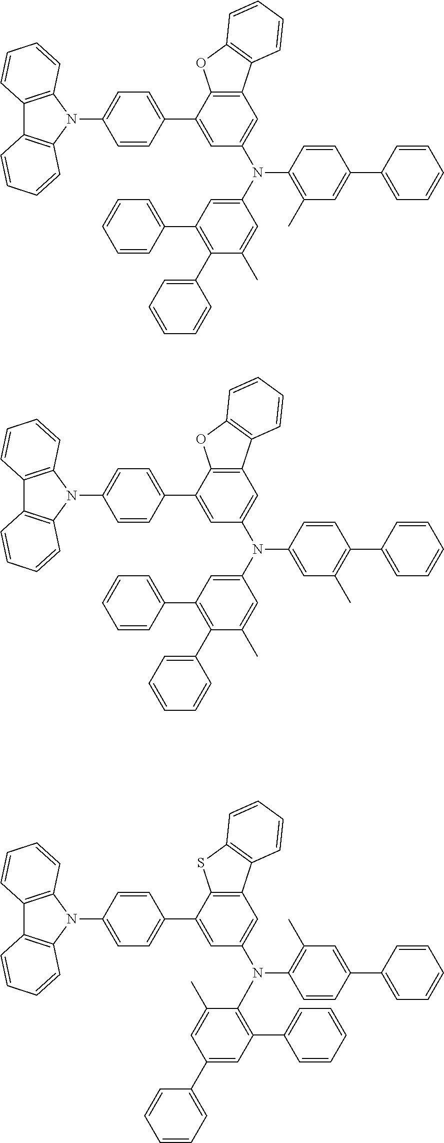 [SMILES: Cc1cc(-c2ccccc2)ccc1N(c1cc(C)c(-c2ccccc2)c(-c2ccccc2)c1)c1cc(-c2ccc(-n3c4ccccc4c4ccccc43)cc2)c2oc3ccccc3c2c1 ; Cc1cc(N(c2cc(C)c(-c3ccccc3)c(-c3ccccc3)c2)c2cc(-c3ccc(-n4c5ccccc5c5ccccc54)cc3)c3oc4ccccc4c3c2)ccc1-c1ccccc1 ; Cc1cc(-c2ccccc2)ccc1N(c1cc(-c2ccc(-n3c4ccccc4c4ccccc43)cc2)c2sc3ccccc3c2c1)c1c(C)cc(-c2ccccc2)cc1-c1ccccc1]

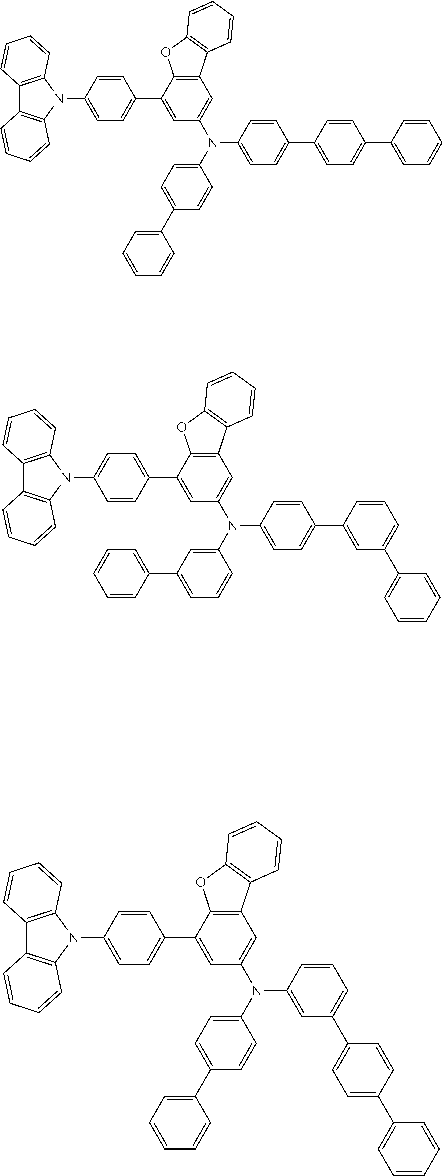 [SMILES: c1ccc(-c2ccc(-c3ccc(N(c4ccc(-c5ccccc5)cc4)c4cc(-c5ccc(-n6c7ccccc7c7ccccc76)cc5)c5oc6ccccc6c5c4)cc3)cc2)cc1 ; c1ccc(-c2cccc(-c3ccc(N(c4cccc(-c5ccccc5)c4)c4cc(-c5ccc(-n6c7ccccc7c7ccccc76)cc5)c5oc6ccccc6c5c4)cc3)c2)cc1 ; c1ccc(-c2ccc(-c3cccc(N(c4ccc(-c5ccccc5)cc4)c4cc(-c5ccc(-n6c7ccccc7c7ccccc76)cc5)c5oc6ccccc6c5c4)c3)cc2)cc1]

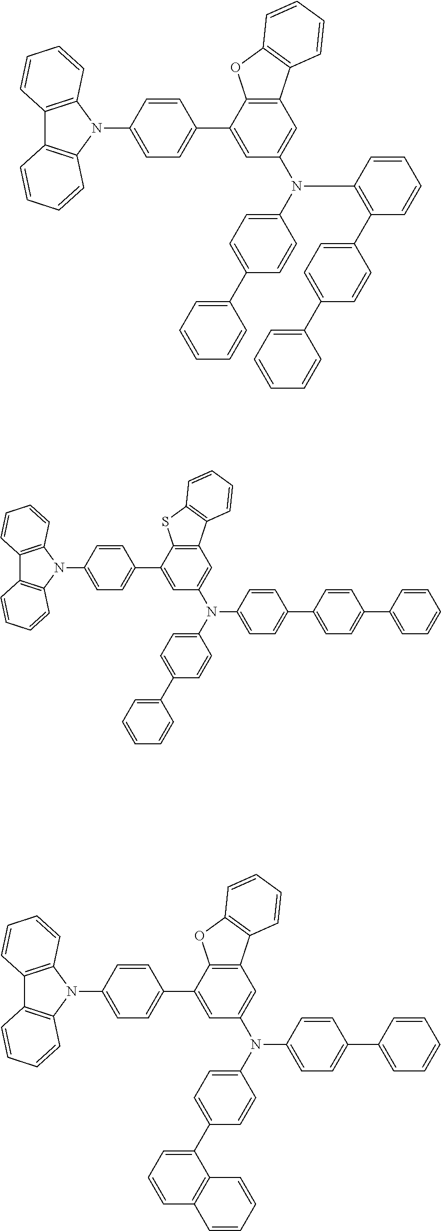 [SMILES: c1ccc(-c2ccc(-c3ccccc3N(c3ccc(-c4ccccc4)cc3)c3cc(-c4ccc(-n5c6ccccc6c6ccccc65)cc4)c4oc5ccccc5c4c3)cc2)cc1 ; c1ccc(-c2ccc(-c3ccc(N(c4ccc(-c5ccccc5)cc4)c4cc(-c5ccc(-n6c7ccccc7c7ccccc76)cc5)c5sc6ccccc6c5c4)cc3)cc2)cc1 ; c1ccc(-c2ccc(N(c3ccc(-c4cccc5ccccc45)cc3)c3cc(-c4ccc(-n5c6ccccc6c6ccccc65)cc4)c4oc5ccccc5c4c3)cc2)cc1]

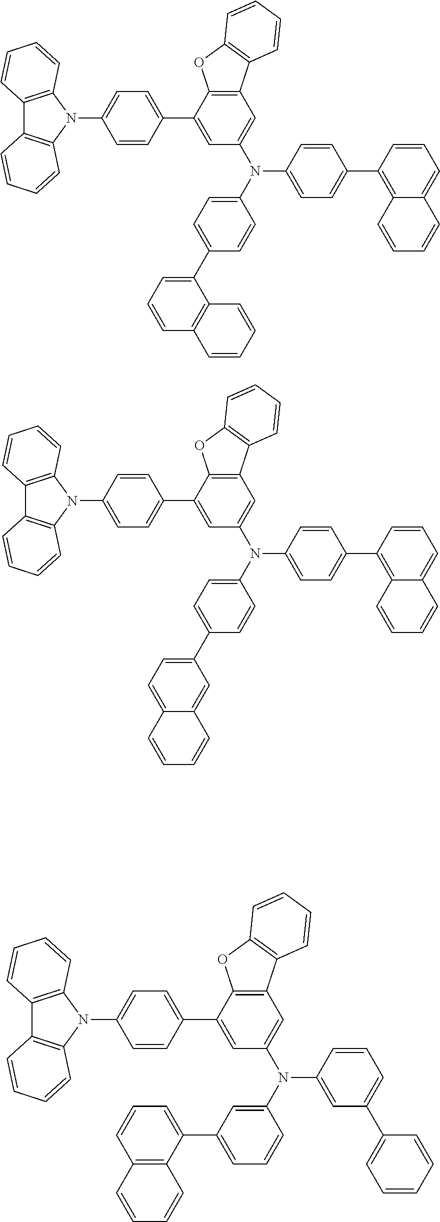

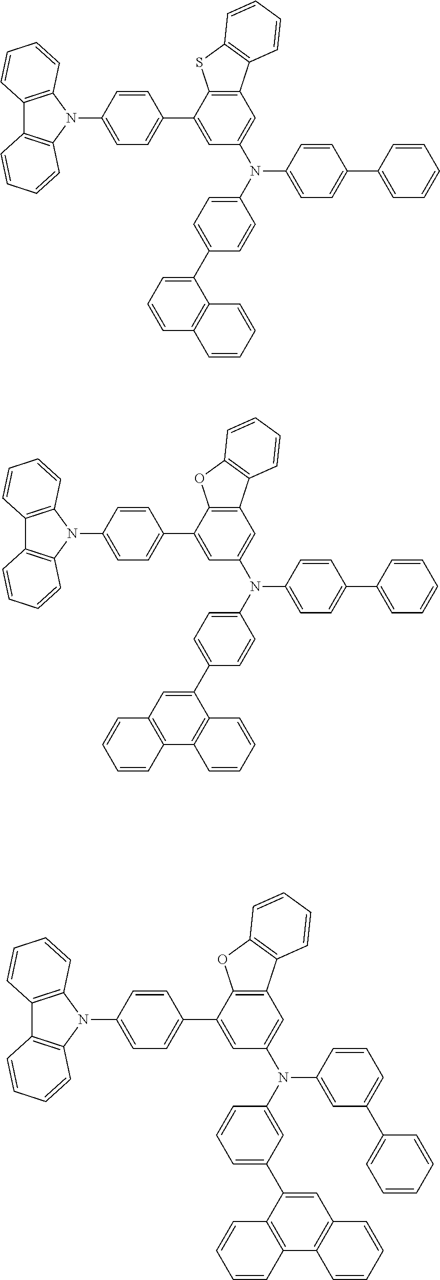 [SMILES: c1ccc(-c2ccc(N(c3ccc(-c4cccc5ccccc45)cc3)c3cc(-c4ccc(-n5c6ccccc6c6ccccc65)cc4)c4sc5ccccc5c4c3)cc2)cc1 ; c1ccc(-c2ccc(N(c3ccc(-c4cc5ccccc5c5ccccc45)cc3)c3cc(-c4ccc(-n5c6ccccc6c6ccccc65)cc4)c4oc5ccccc5c4c3)cc2)cc1 ; c1ccc(-c2cccc(N(c3cccc(-c4cc5ccccc5c5ccccc45)c3)c3cc(-c4ccc(-n5c6ccccc6c6ccccc65)cc4)c4oc5ccccc5c4c3)c2)cc1]

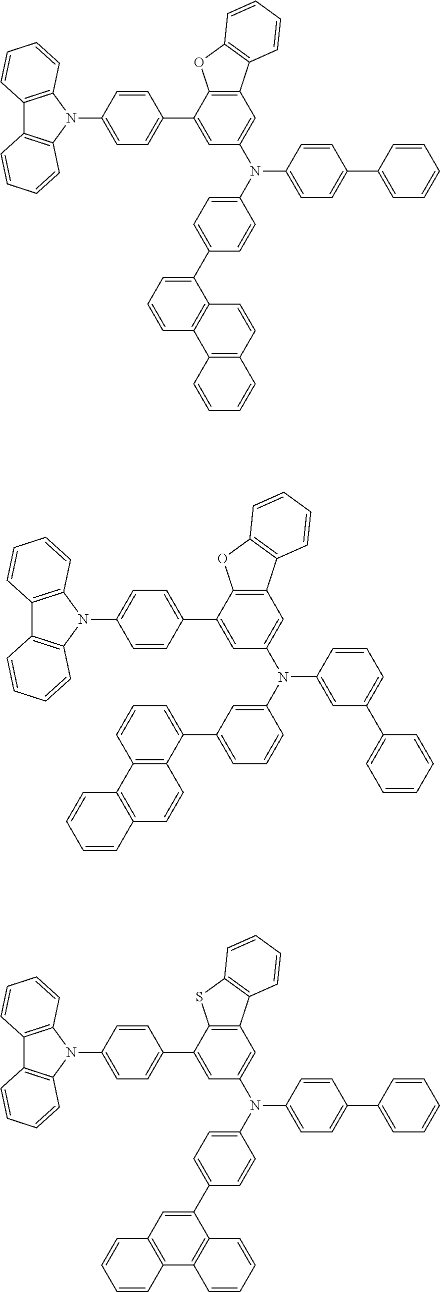 [SMILES: c1ccc(-c2ccc(N(c3ccc(-c4cccc5c4ccc4ccccc45)cc3)c3cc(-c4ccc(-n5c6ccccc6c6ccccc65)cc4)c4oc5ccccc5c4c3)cc2)cc1 ; c1ccc(-c2cccc(N(c3cccc(-c4cccc5c4ccc4ccccc45)c3)c3cc(-c4ccc(-n5c6ccccc6c6ccccc65)cc4)c4oc5ccccc5c4c3)c2)cc1 ; c1ccc(-c2ccc(N(c3ccc(-c4cc5ccccc5c5ccccc45)cc3)c3cc(-c4ccc(-n5c6ccccc6c6ccccc65)cc4)c4sc5ccccc5c4c3)cc2)cc1]

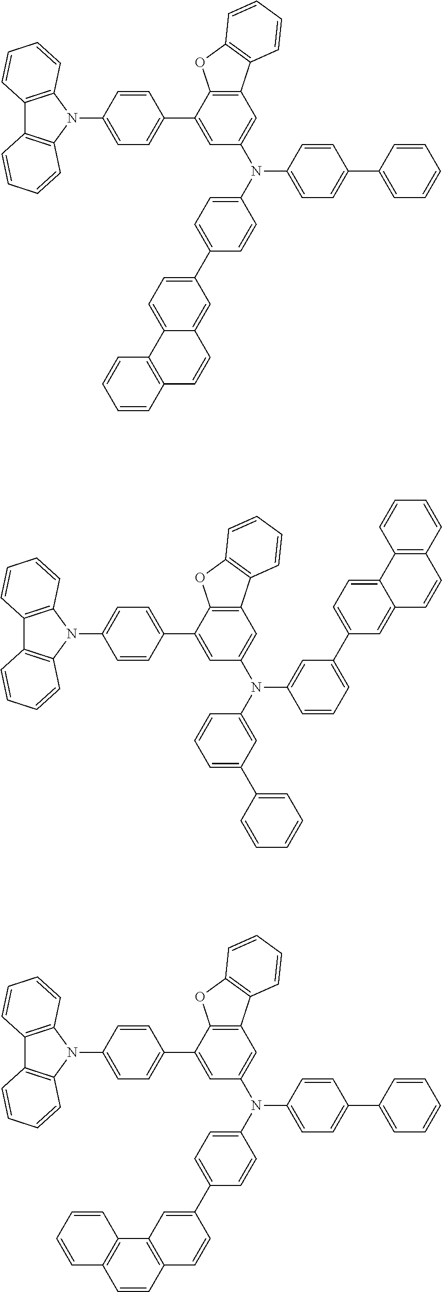 [SMILES: c1ccc(-c2ccc(N(c3ccc(-c4ccc5c(ccc6ccccc65)c4)cc3)c3cc(-c4ccc(-n5c6ccccc6c6ccccc65)cc4)c4oc5ccccc5c4c3)cc2)cc1 ; c1ccc(-c2cccc(N(c3cccc(-c4ccc5c(ccc6ccccc65)c4)c3)c3cc(-c4ccc(-n5c6ccccc6c6ccccc65)cc4)c4oc5ccccc5c4c3)c2)cc1 ; c1ccc(-c2ccc(N(c3ccc(-c4ccc5ccc6ccccc6c5c4)cc3)c3cc(-c4ccc(-n5c6ccccc6c6ccccc65)cc4)c4oc5ccccc5c4c3)cc2)cc1]

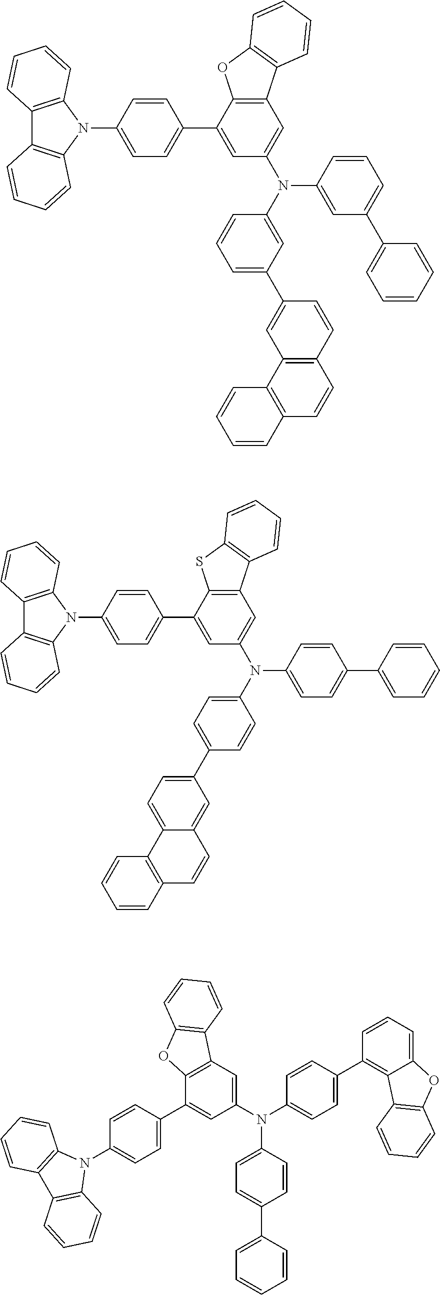 [SMILES: c1ccc(-c2cccc(N(c3cccc(-c4ccc5ccc6ccccc6c5c4)c3)c3cc(-c4ccc(-n5c6ccccc6c6ccccc65)cc4)c4oc5ccccc5c4c3)c2)cc1 ; c1ccc(-c2ccc(N(c3ccc(-c4ccc5c(ccc6ccccc65)c4)cc3)c3cc(-c4ccc(-n5c6ccccc6c6ccccc65)cc4)c4sc5ccccc5c4c3)cc2)cc1 ; c1ccc(-c2ccc(N(c3ccc(-c4cccc5oc6ccccc6c45)cc3)c3cc(-c4ccc(-n5c6ccccc6c6ccccc65)cc4)c4oc5ccccc5c4c3)cc2)cc1]

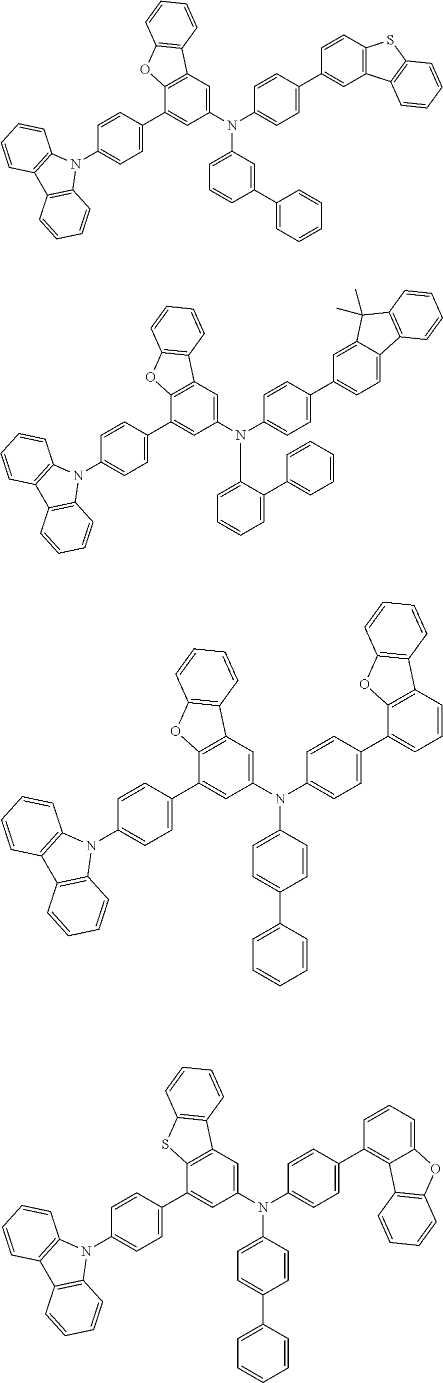 [SMILES: c1ccc(-c2cccc(N(c3ccc(-c4ccc5sc6ccccc6c5c4)cc3)c3cc(-c4ccc(-n5c6ccccc6c6ccccc65)cc4)c4oc5ccccc5c4c3)c2)cc1 ; CC1(C)c2ccccc2-c2ccc(-c3ccc(N(c4cc(-c5ccc(-n6c7ccccc7c7ccccc76)cc5)c5oc6ccccc6c5c4)c4ccccc4-c4ccccc4)cc3)cc21 ; c1ccc(-c2ccc(N(c3ccc(-c4cccc5c4oc4ccccc45)cc3)c3cc(-c4ccc(-n5c6ccccc6c6ccccc65)cc4)c4oc5ccccc5c4c3)cc2)cc1 ; c1ccc(-c2ccc(N(c3ccc(-c4cccc5oc6ccccc6c45)cc3)c3cc(-c4ccc(-n5c6ccccc6c6ccccc65)cc4)c4sc5ccccc5c4c3)cc2)cc1]

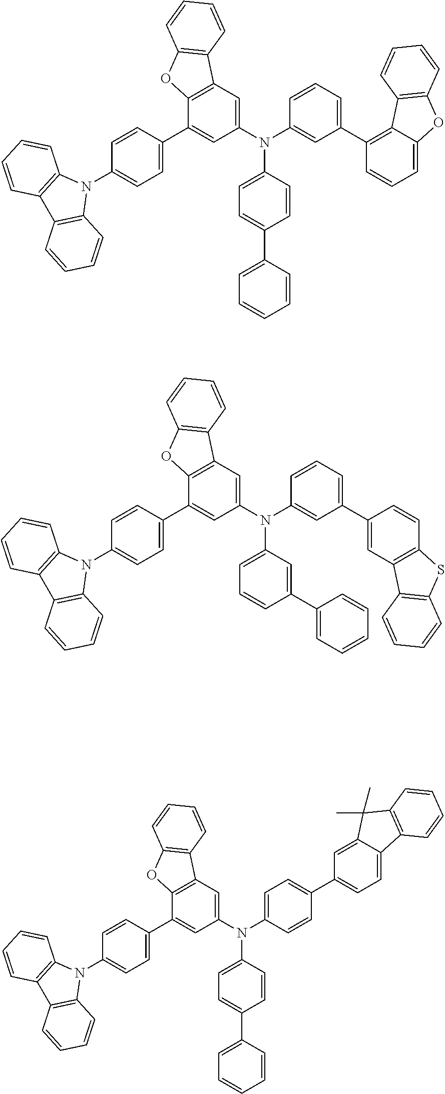 [SMILES: c1ccc(-c2ccc(N(c3cccc(-c4cccc5oc6ccccc6c45)c3)c3cc(-c4ccc(-n5c6ccccc6c6ccccc65)cc4)c4oc5ccccc5c4c3)cc2)cc1 ; c1ccc(-c2cccc(N(c3cccc(-c4ccc5sc6ccccc6c5c4)c3)c3cc(-c4ccc(-n5c6ccccc6c6ccccc65)cc4)c4oc5ccccc5c4c3)c2)cc1 ; CC1(C)c2ccccc2-c2ccc(-c3ccc(N(c4ccc(-c5ccccc5)cc4)c4cc(-c5ccc(-n6c7ccccc7c7ccccc76)cc5)c5oc6ccccc6c5c4)cc3)cc21]

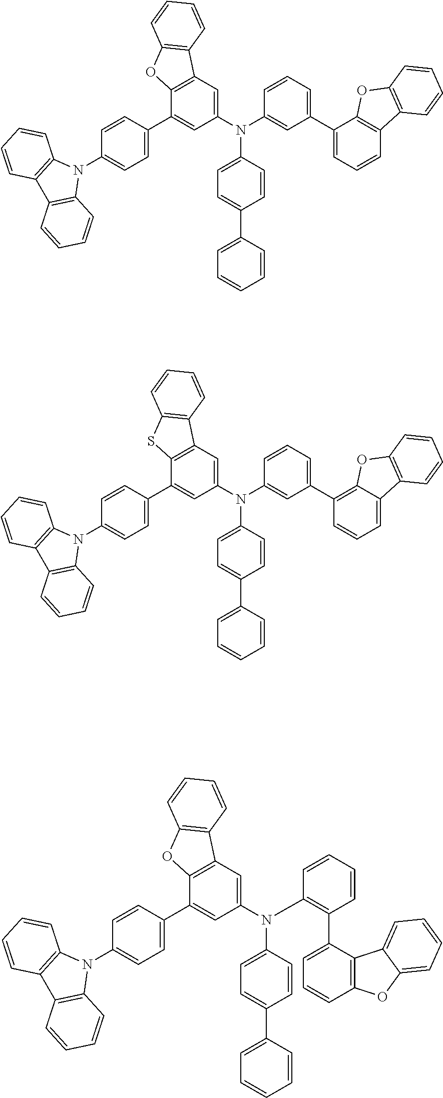 [SMILES: c1ccc(-c2ccc(N(c3cccc(-c4cccc5c4oc4ccccc45)c3)c3cc(-c4ccc(-n5c6ccccc6c6ccccc65)cc4)c4oc5ccccc5c4c3)cc2)cc1 ; c1ccc(-c2ccc(N(c3cccc(-c4cccc5c4oc4ccccc45)c3)c3cc(-c4ccc(-n5c6ccccc6c6ccccc65)cc4)c4sc5ccccc5c4c3)cc2)cc1 ; c1ccc(-c2ccc(N(c3cc(-c4ccc(-n5c6ccccc6c6ccccc65)cc4)c4oc5ccccc5c4c3)c3ccccc3-c3cccc4oc5ccccc5c34)cc2)cc1]

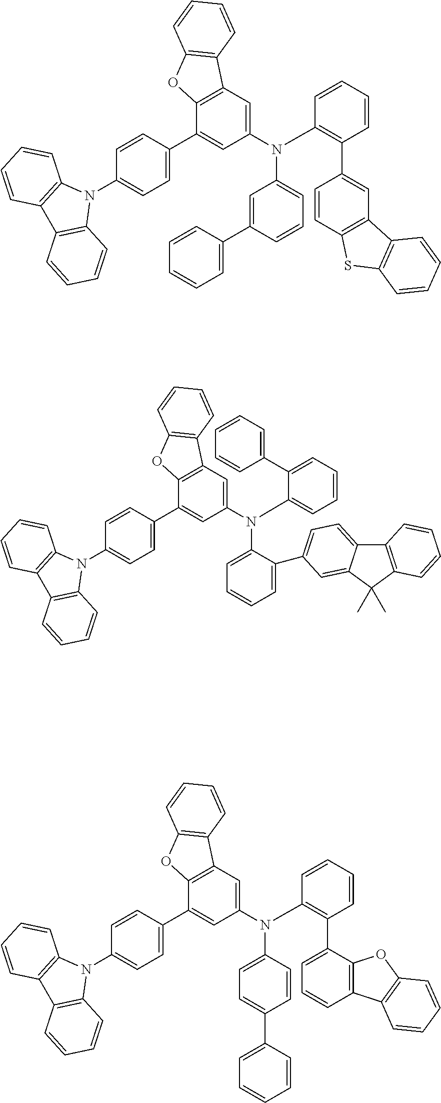 [SMILES: c1ccc(-c2cccc(N(c3cc(-c4ccc(-n5c6ccccc6c6ccccc65)cc4)c4oc5ccccc5c4c3)c3ccccc3-c3ccc4sc5ccccc5c4c3)c2)cc1 ; CC1(C)c2ccccc2-c2ccc(-c3ccccc3N(c3cc(-c4ccc(-n5c6ccccc6c6ccccc65)cc4)c4oc5ccccc5c4c3)c3ccccc3-c3ccccc3)cc21 ; c1ccc(-c2ccc(N(c3cc(-c4ccc(-n5c6ccccc6c6ccccc65)cc4)c4oc5ccccc5c4c3)c3ccccc3-c3cccc4c3oc3ccccc34)cc2)cc1]

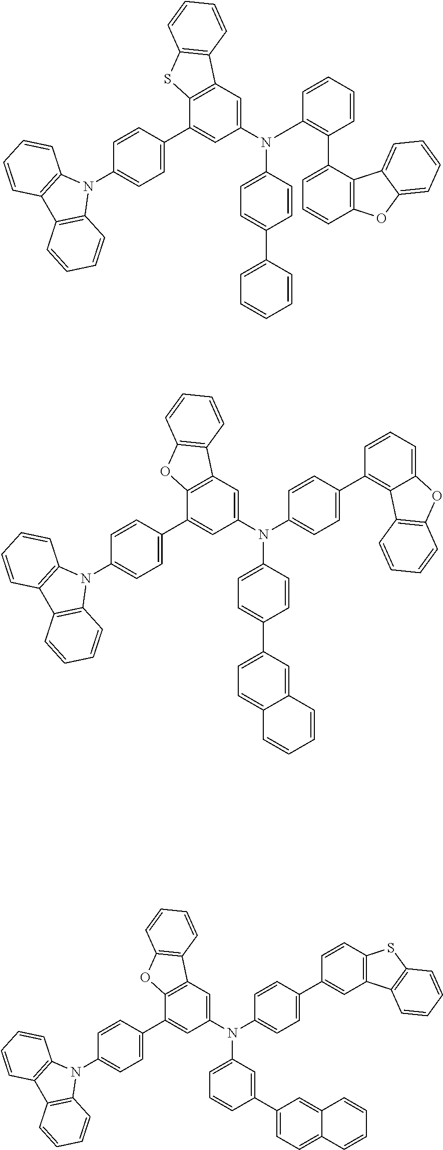 [SMILES: c1ccc(-c2ccc(N(c3cc(-c4ccc(-n5c6ccccc6c6ccccc65)cc4)c4sc5ccccc5c4c3)c3ccccc3-c3cccc4oc5ccccc5c34)cc2)cc1 ; c1ccc2cc(-c3ccc(N(c4ccc(-c5cccc6oc7ccccc7c56)cc4)c4cc(-c5ccc(-n6c7ccccc7c7ccccc76)cc5)c5oc6ccccc6c5c4)cc3)ccc2c1 ; c1cc(-c2ccc3ccccc3c2)cc(N(c2ccc(-c3ccc4sc5ccccc5c4c3)cc2)c2cc(-c3ccc(-n4c5ccccc5c5ccccc54)cc3)c3oc4ccccc4c3c2)c1]

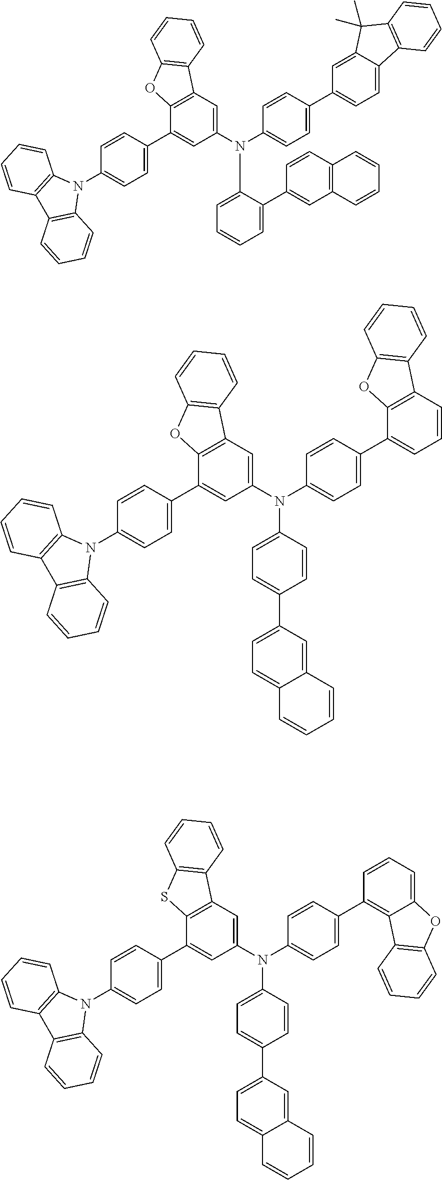 [SMILES: CC1(C)c2ccccc2-c2ccc(-c3ccc(N(c4cc(-c5ccc(-n6c7ccccc7c7ccccc76)cc5)c5oc6ccccc6c5c4)c4ccccc4-c4ccc5ccccc5c4)cc3)cc21 ; c1ccc2cc(-c3ccc(N(c4ccc(-c5cccc6c5oc5ccccc56)cc4)c4cc(-c5ccc(-n6c7ccccc7c7ccccc76)cc5)c5oc6ccccc6c5c4)cc3)ccc2c1 ; c1ccc2cc(-c3ccc(N(c4ccc(-c5cccc6oc7ccccc7c56)cc4)c4cc(-c5ccc(-n6c7ccccc7c7ccccc76)cc5)c5sc6ccccc6c5c4)cc3)ccc2c1]

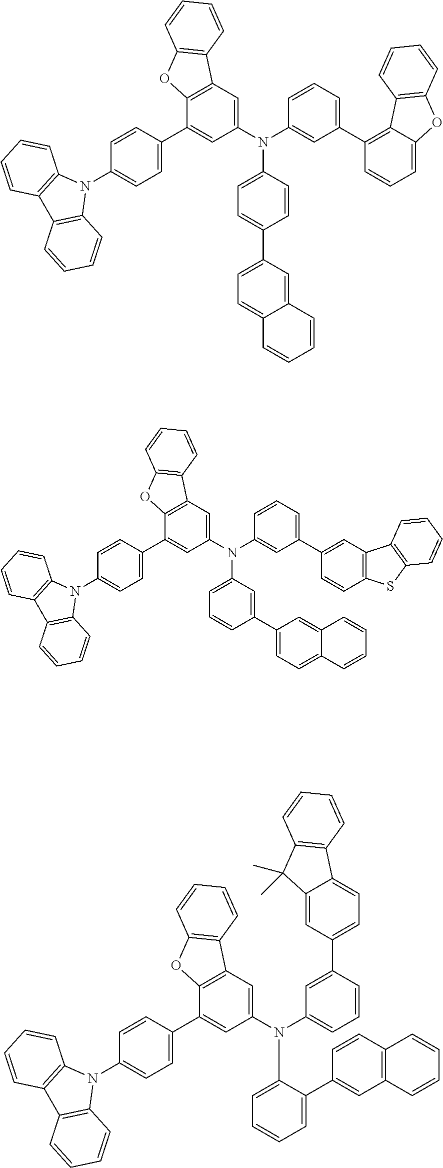 [SMILES: c1cc(-c2cccc3oc4ccccc4c23)cc(N(c2ccc(-c3ccc4ccccc4c3)cc2)c2cc(-c3ccc(-n4c5ccccc5c5ccccc54)cc3)c3oc4ccccc4c3c2)c1 ; c1cc(-c2ccc3ccccc3c2)cc(N(c2cccc(-c3ccc4sc5ccccc5c4c3)c2)c2cc(-c3ccc(-n4c5ccccc5c5ccccc54)cc3)c3oc4ccccc4c3c2)c1 ; CC1(C)c2ccccc2-c2ccc(-c3cccc(N(c4cc(-c5ccc(-n6c7ccccc7c7ccccc76)cc5)c5oc6ccccc6c5c4)c4ccccc4-c4ccc5ccccc5c4)c3)cc21]

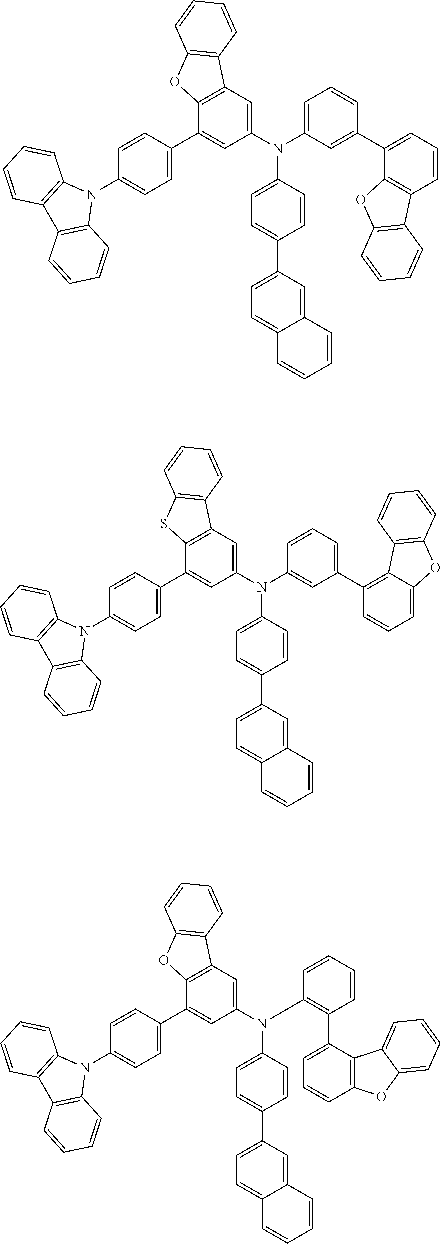 [SMILES: c1cc(-c2cccc3c2oc2ccccc23)cc(N(c2ccc(-c3ccc4ccccc4c3)cc2)c2cc(-c3ccc(-n4c5ccccc5c5ccccc54)cc3)c3oc4ccccc4c3c2)c1 ; c1cc(-c2cccc3oc4ccccc4c23)cc(N(c2ccc(-c3ccc4ccccc4c3)cc2)c2cc(-c3ccc(-n4c5ccccc5c5ccccc54)cc3)c3sc4ccccc4c3c2)c1 ; c1ccc(N(c2ccc(-c3ccc4ccccc4c3)cc2)c2cc(-c3ccc(-n4c5ccccc5c5ccccc54)cc3)c3oc4ccccc4c3c2)c(-c2cccc3oc4ccccc4c23)c1]

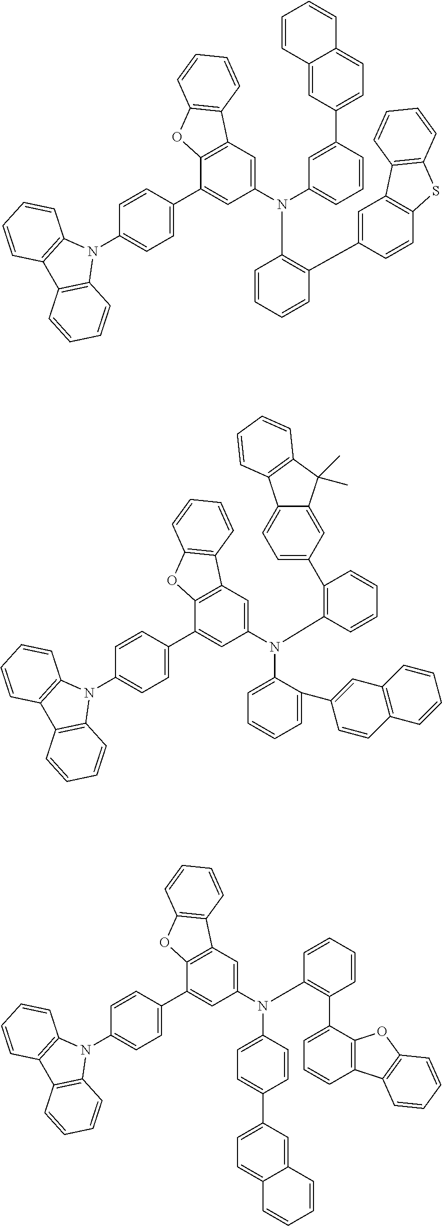 [SMILES: c1cc(-c2ccc3ccccc3c2)cc(N(c2cc(-c3ccc(-n4c5ccccc5c5ccccc54)cc3)c3oc4ccccc4c3c2)c2ccccc2-c2ccc3sc4ccccc4c3c2)c1 ; CC1(C)c2ccccc2-c2ccc(-c3ccccc3N(c3cc(-c4ccc(-n5c6ccccc6c6ccccc65)cc4)c4oc5ccccc5c4c3)c3ccccc3-c3ccc4ccccc4c3)cc21 ; c1ccc(N(c2ccc(-c3ccc4ccccc4c3)cc2)c2cc(-c3ccc(-n4c5ccccc5c5ccccc54)cc3)c3oc4ccccc4c3c2)c(-c2cccc3c2oc2ccccc23)c1]

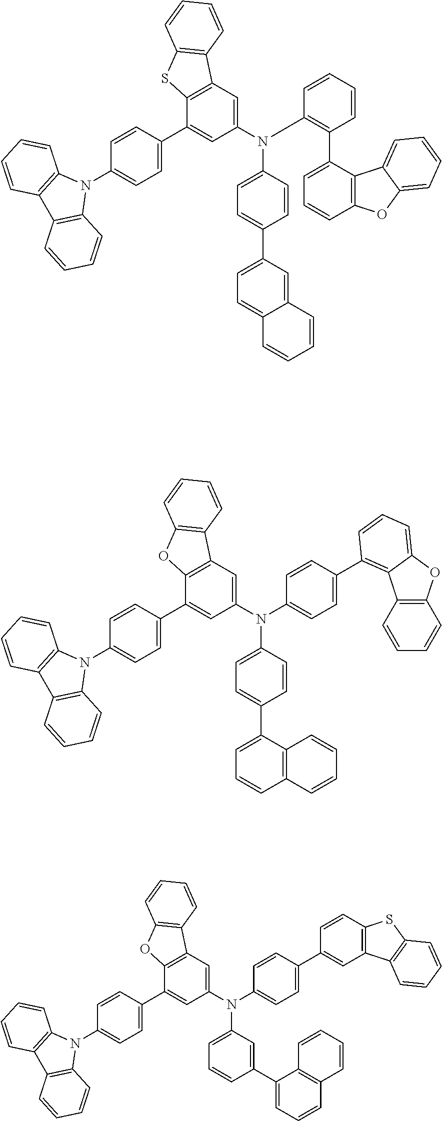 [SMILES: c1ccc(N(c2ccc(-c3ccc4ccccc4c3)cc2)c2cc(-c3ccc(-n4c5ccccc5c5ccccc54)cc3)c3sc4ccccc4c3c2)c(-c2cccc3oc4ccccc4c23)c1 ; c1ccc2c(-c3ccc(N(c4ccc(-c5cccc6oc7ccccc7c56)cc4)c4cc(-c5ccc(-n6c7ccccc7c7ccccc76)cc5)c5oc6ccccc6c5c4)cc3)cccc2c1 ; c1cc(-c2cccc3ccccc23)cc(N(c2ccc(-c3ccc4sc5ccccc5c4c3)cc2)c2cc(-c3ccc(-n4c5ccccc5c5ccccc54)cc3)c3oc4ccccc4c3c2)c1]

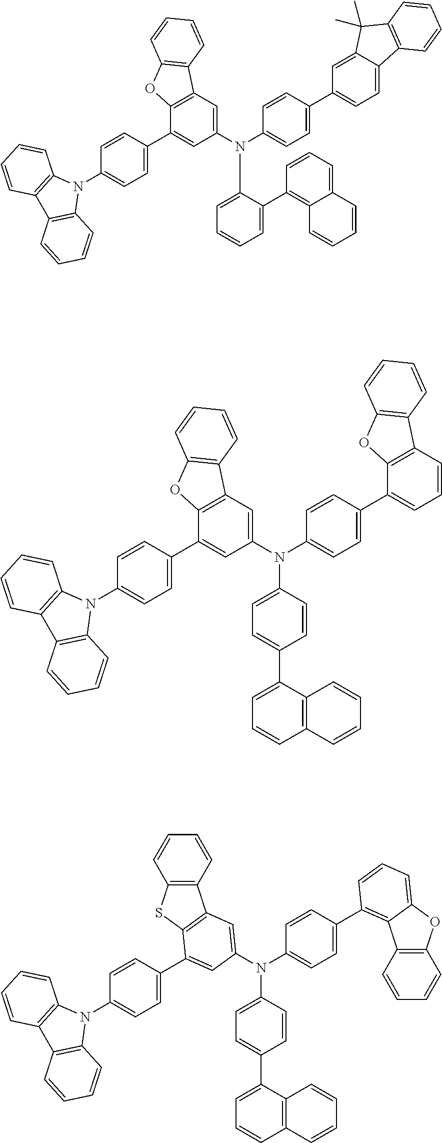 [SMILES: CC1(C)c2ccccc2-c2ccc(-c3ccc(N(c4cc(-c5ccc(-n6c7ccccc7c7ccccc76)cc5)c5oc6ccccc6c5c4)c4ccccc4-c4cccc5ccccc45)cc3)cc21 ; c1ccc2c(-c3ccc(N(c4ccc(-c5cccc6c5oc5ccccc56)cc4)c4cc(-c5ccc(-n6c7ccccc7c7ccccc76)cc5)c5oc6ccccc6c5c4)cc3)cccc2c1 ; c1ccc2c(-c3ccc(N(c4ccc(-c5cccc6oc7ccccc7c56)cc4)c4cc(-c5ccc(-n6c7ccccc7c7ccccc76)cc5)c5sc6ccccc6c5c4)cc3)cccc2c1]

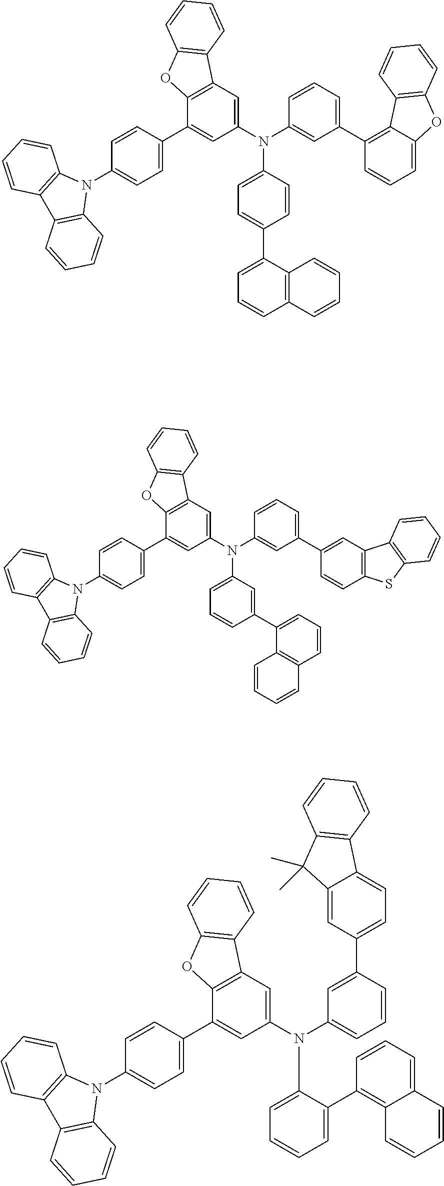 [SMILES: c1cc(-c2cccc3oc4ccccc4c23)cc(N(c2ccc(-c3cccc4ccccc34)cc2)c2cc(-c3ccc(-n4c5ccccc5c5ccccc54)cc3)c3oc4ccccc4c3c2)c1 ; c1cc(-c2ccc3sc4ccccc4c3c2)cc(N(c2cccc(-c3cccc4ccccc34)c2)c2cc(-c3ccc(-n4c5ccccc5c5ccccc54)cc3)c3oc4ccccc4c3c2)c1 ; CC1(C)c2ccccc2-c2ccc(-c3cccc(N(c4cc(-c5ccc(-n6c7ccccc7c7ccccc76)cc5)c5oc6ccccc6c5c4)c4ccccc4-c4cccc5ccccc45)c3)cc21]

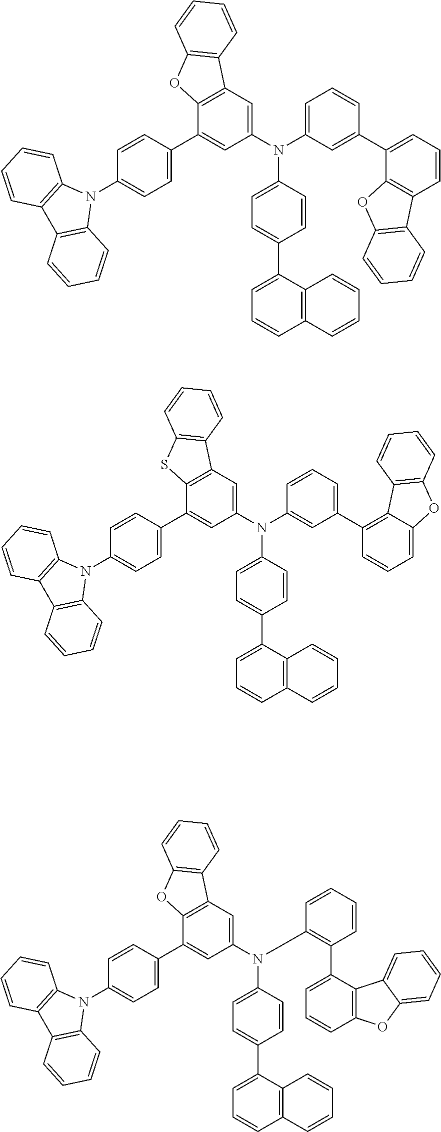 [SMILES: c1cc(-c2cccc3c2oc2ccccc23)cc(N(c2ccc(-c3cccc4ccccc34)cc2)c2cc(-c3ccc(-n4c5ccccc5c5ccccc54)cc3)c3oc4ccccc4c3c2)c1 ; c1cc(-c2cccc3oc4ccccc4c23)cc(N(c2ccc(-c3cccc4ccccc34)cc2)c2cc(-c3ccc(-n4c5ccccc5c5ccccc54)cc3)c3sc4ccccc4c3c2)c1 ; c1ccc(N(c2ccc(-c3cccc4ccccc34)cc2)c2cc(-c3ccc(-n4c5ccccc5c5ccccc54)cc3)c3oc4ccccc4c3c2)c(-c2cccc3oc4ccccc4c23)c1]

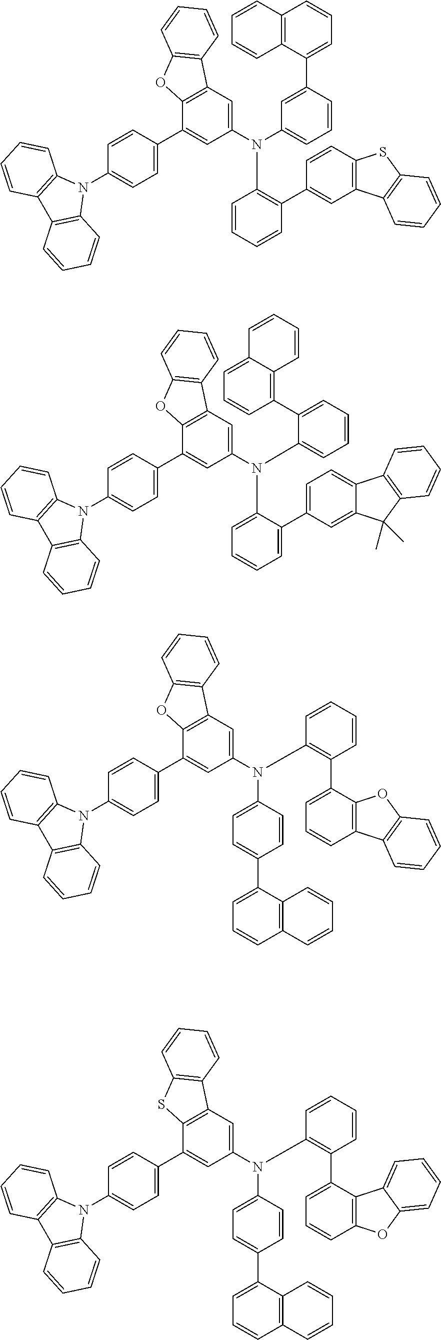 [SMILES: c1cc(-c2cccc3ccccc23)cc(N(c2cc(-c3ccc(-n4c5ccccc5c5ccccc54)cc3)c3oc4ccccc4c3c2)c2ccccc2-c2ccc3sc4ccccc4c3c2)c1 ; CC1(C)c2ccccc2-c2ccc(-c3ccccc3N(c3cc(-c4ccc(-n5c6ccccc6c6ccccc65)cc4)c4oc5ccccc5c4c3)c3ccccc3-c3cccc4ccccc34)cc21 ; c1ccc(N(c2ccc(-c3cccc4ccccc34)cc2)c2cc(-c3ccc(-n4c5ccccc5c5ccccc54)cc3)c3oc4ccccc4c3c2)c(-c2cccc3c2oc2ccccc23)c1 ; c1ccc(N(c2ccc(-c3cccc4ccccc34)cc2)c2cc(-c3ccc(-n4c5ccccc5c5ccccc54)cc3)c3sc4ccccc4c3c2)c(-c2cccc3oc4ccccc4c23)c1]

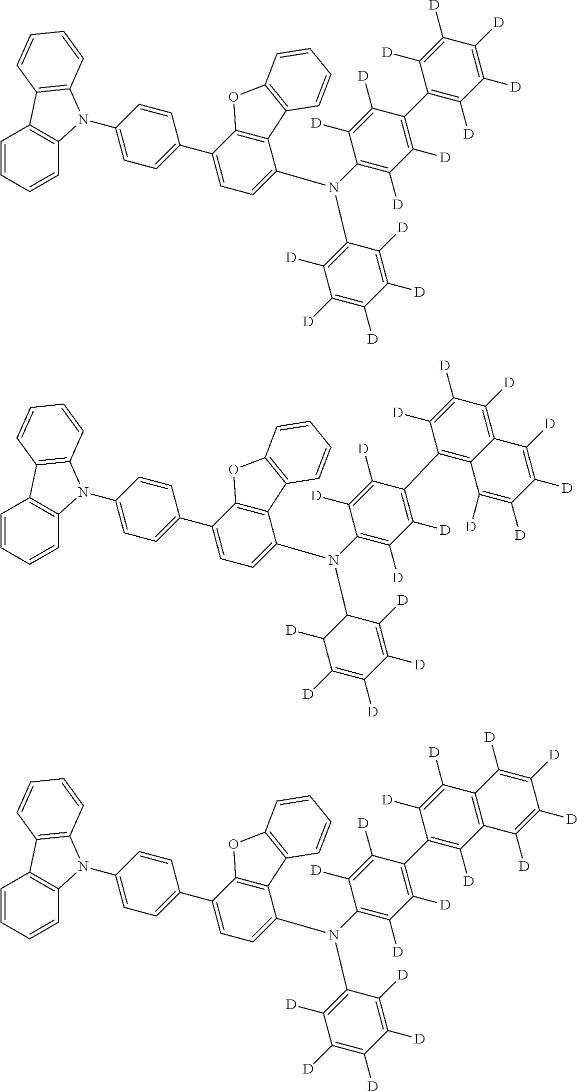

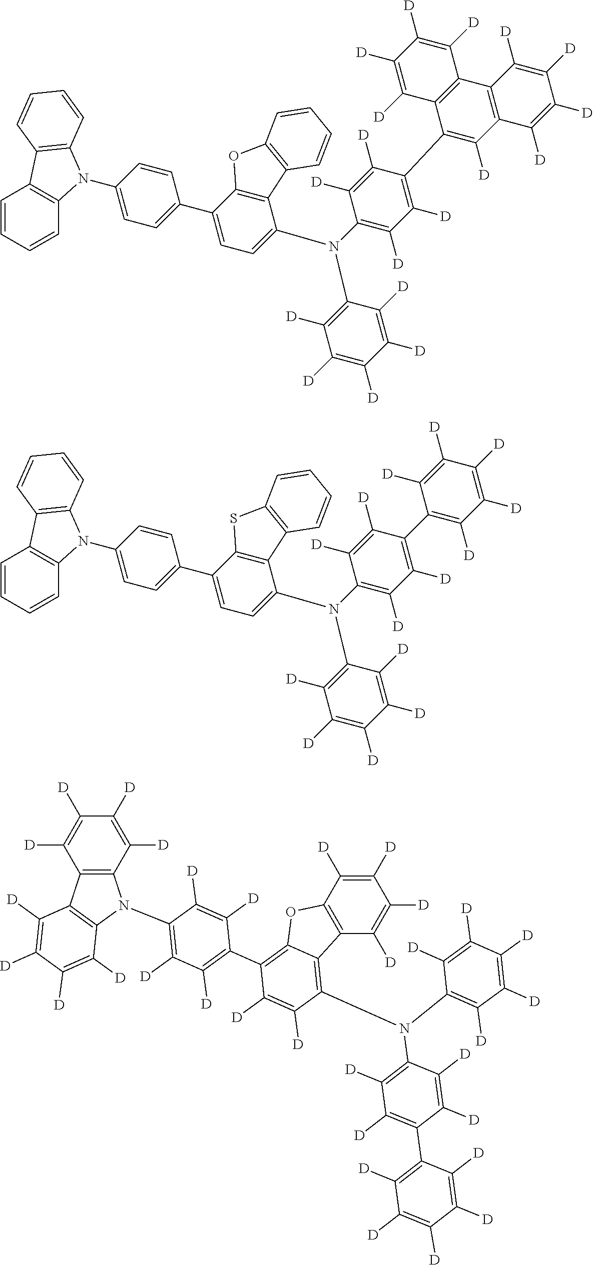

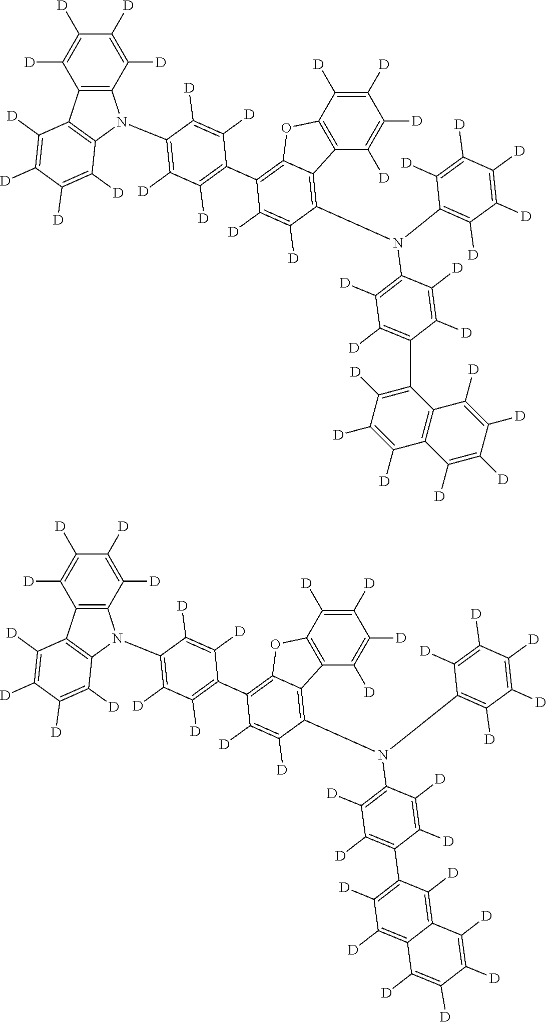

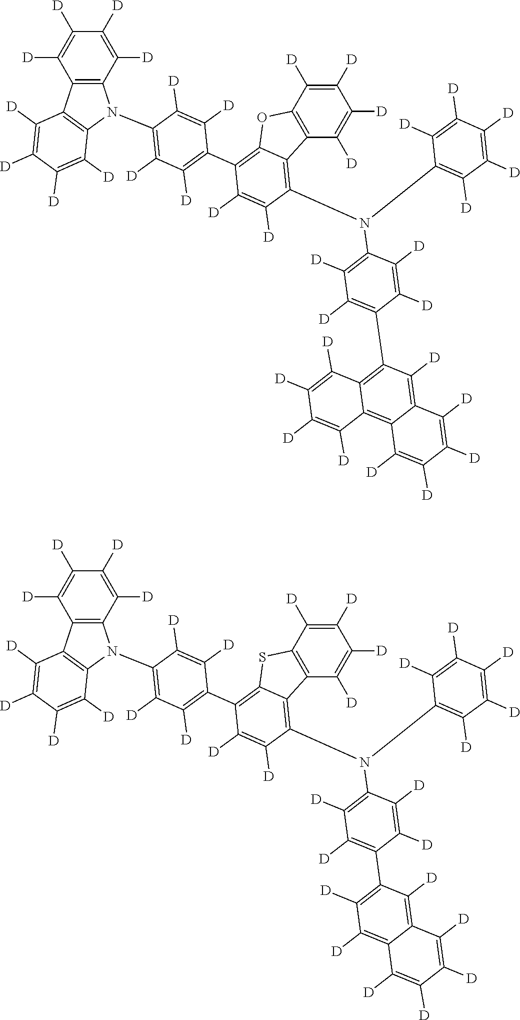

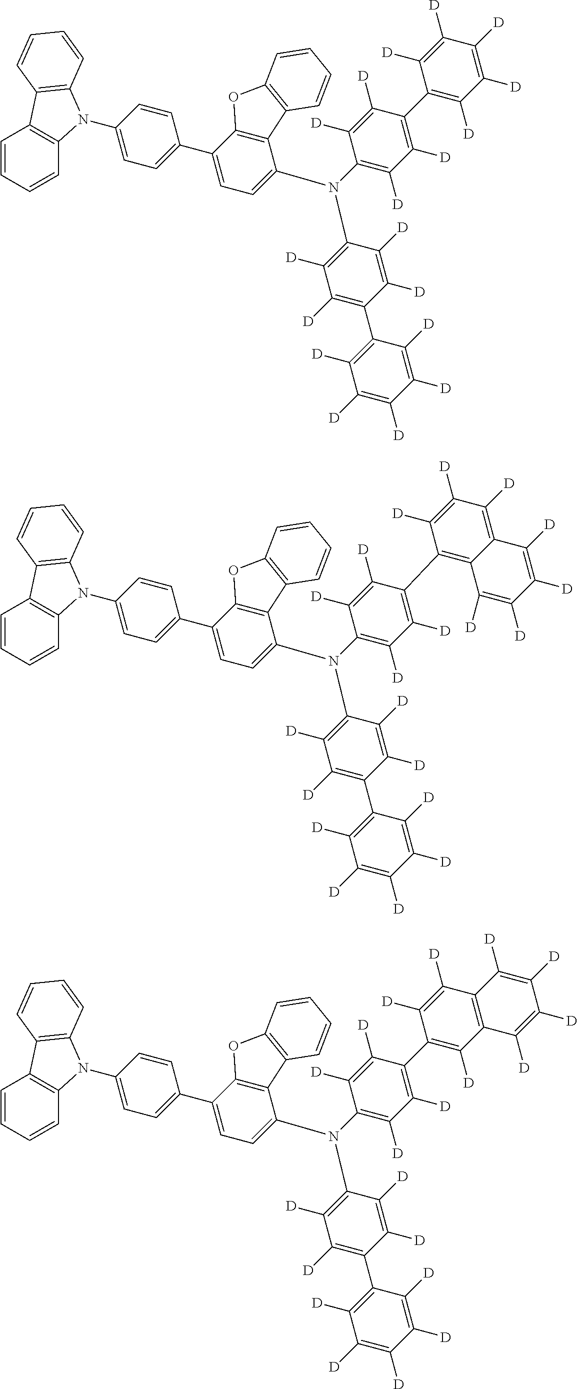

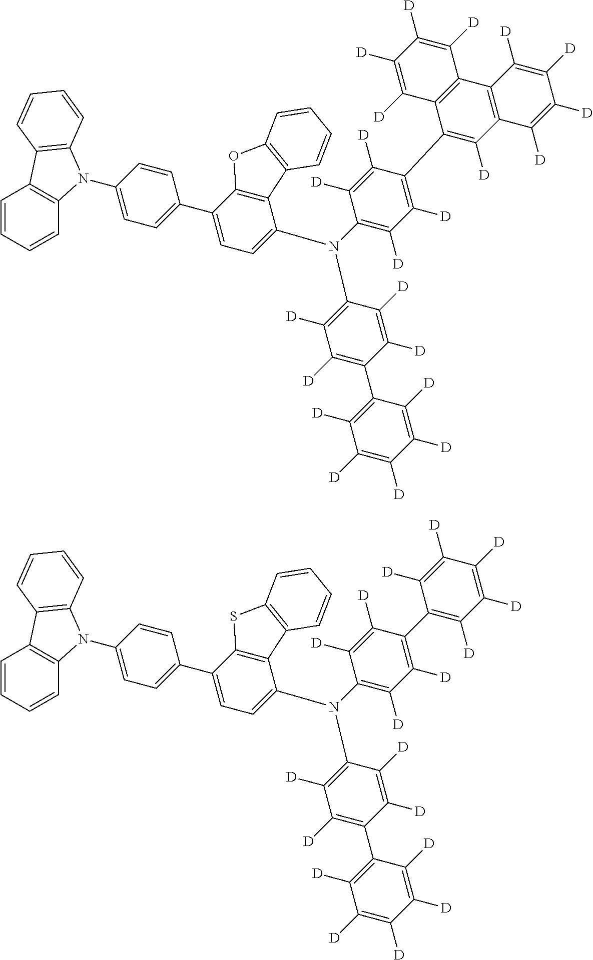 [SMILES: [2H]c1c([2H])c([2H])c(-c2c([2H])c([2H])c(N(c3c([2H])c([2H])c(-c4c([2H])c5c([2H])c([2H])c([2H])c([2H])c5c5c([2H])c([2H])c([2H])c([2H])c45)c([2H])c3[2H])c3ccc(-c4ccc(-n5c6ccccc6c6ccccc65)cc4)c4oc5ccccc5c34)c([2H])c2[2H])c([2H])c1[2H] ; [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3ccc(-c4ccc(-n5c6ccccc6c6ccccc65)cc4)c4sc5ccccc5c34)c([2H])c2[2H])c([2H])c1[2H]]

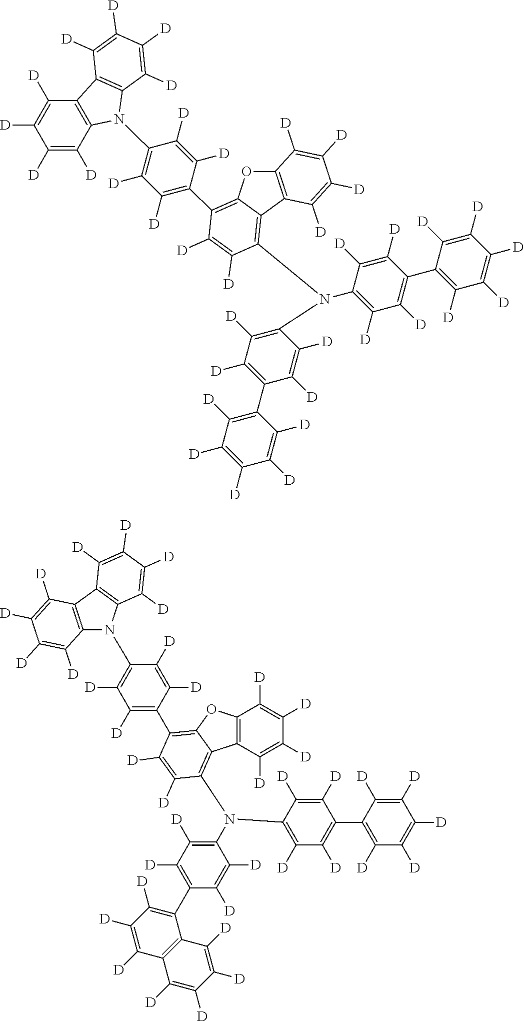

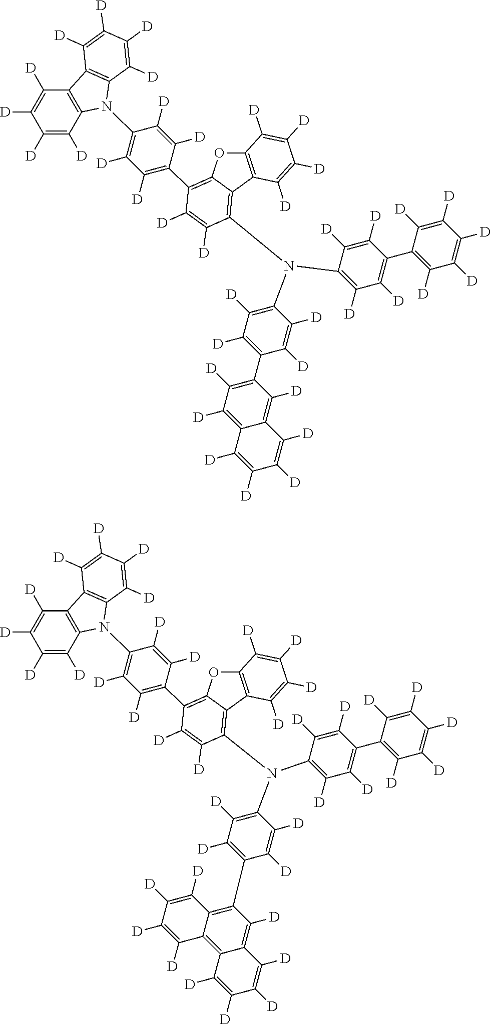

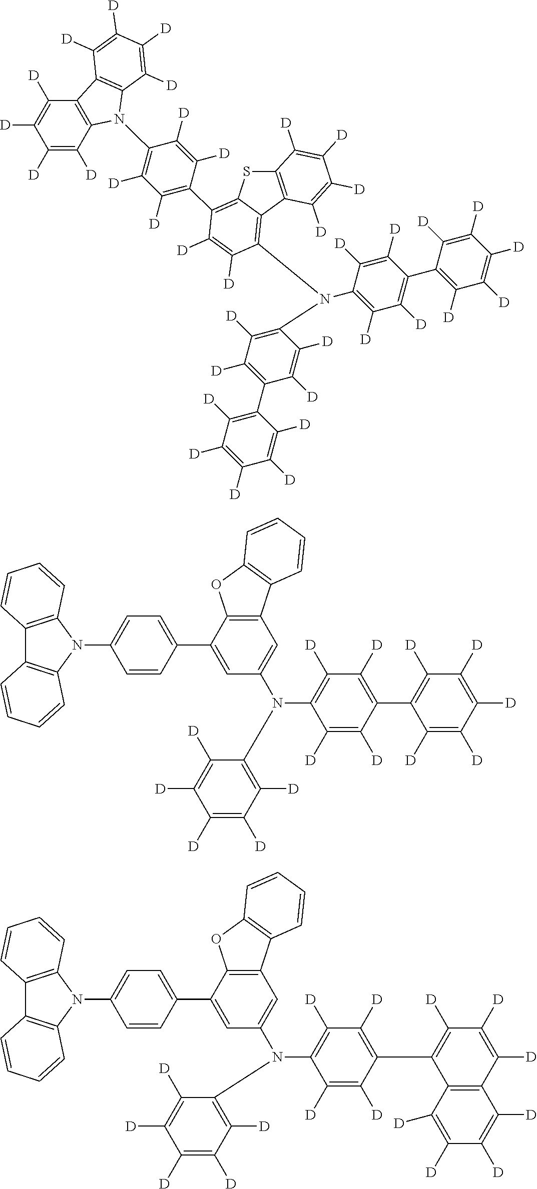

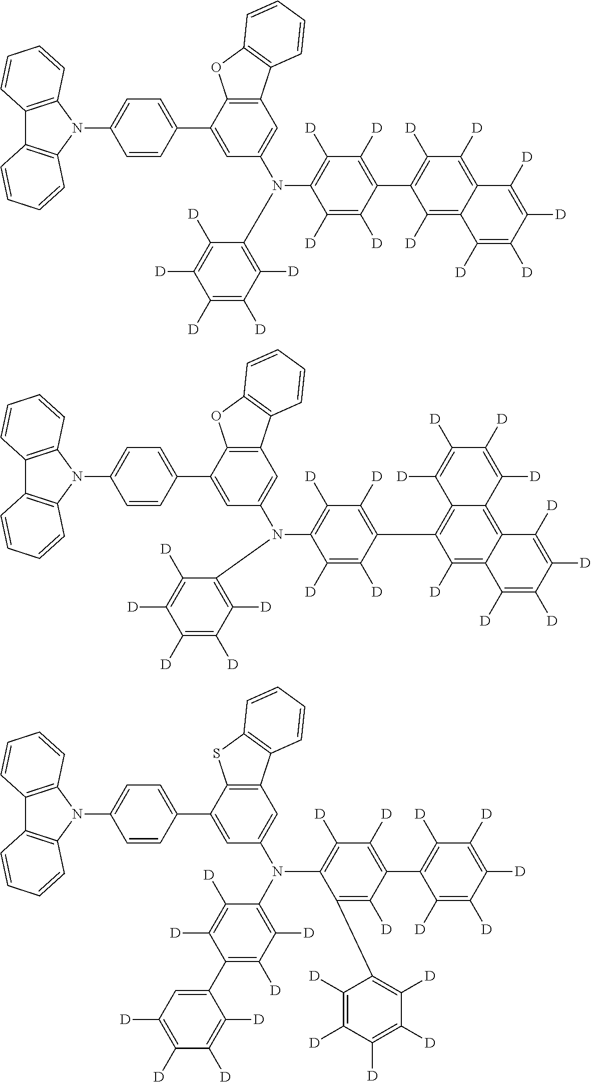

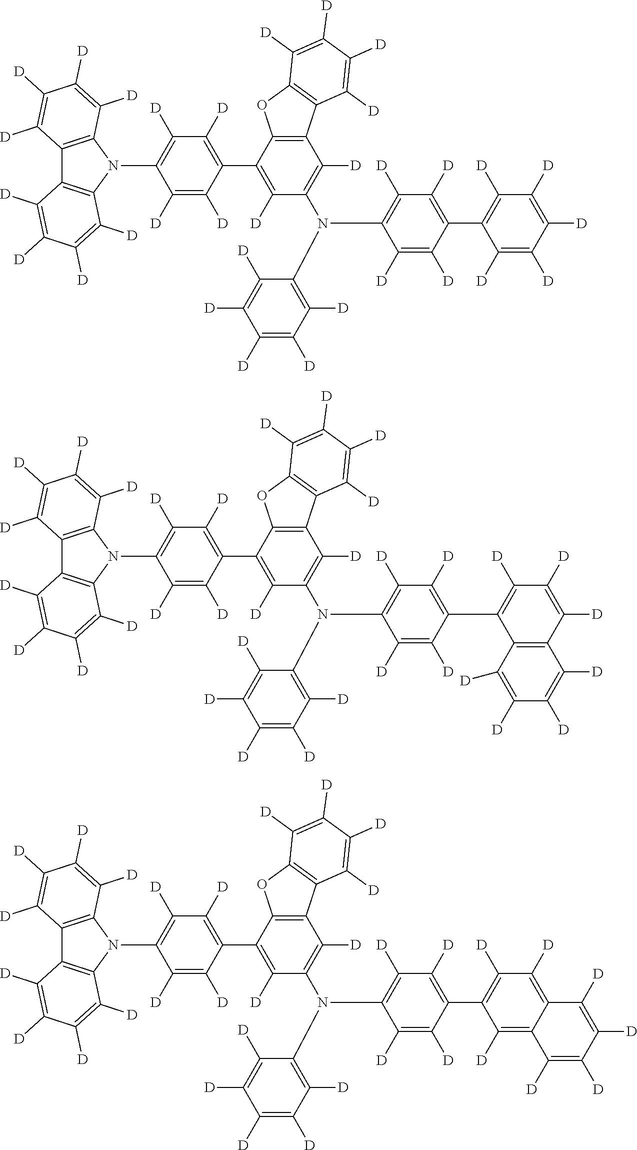

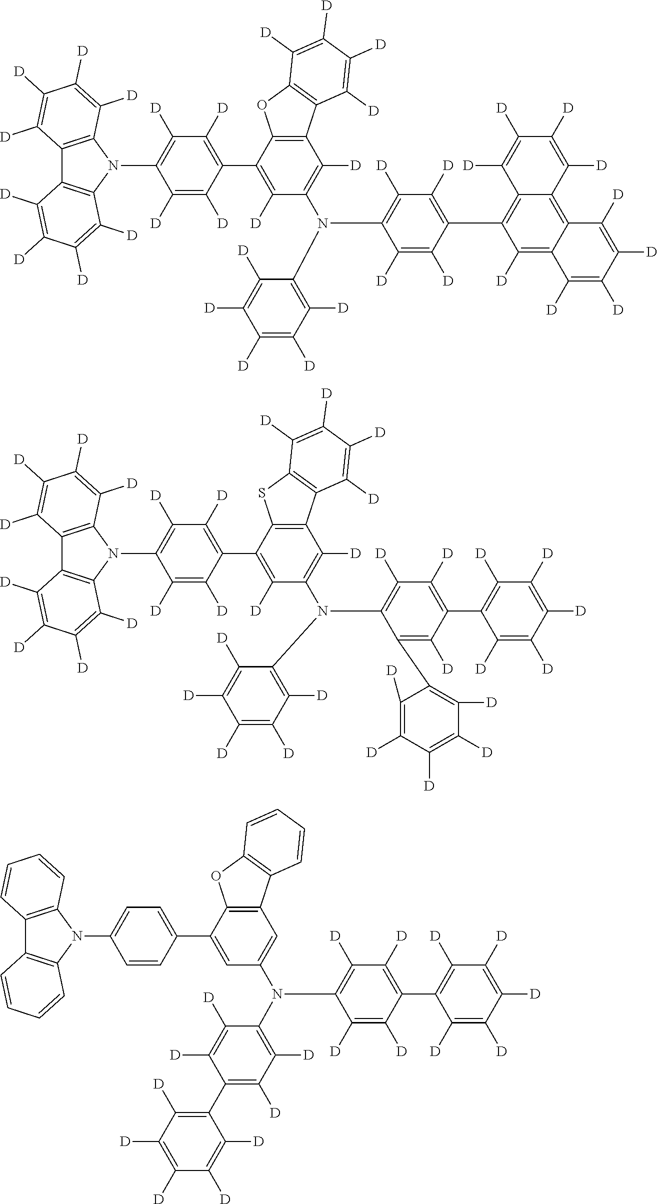

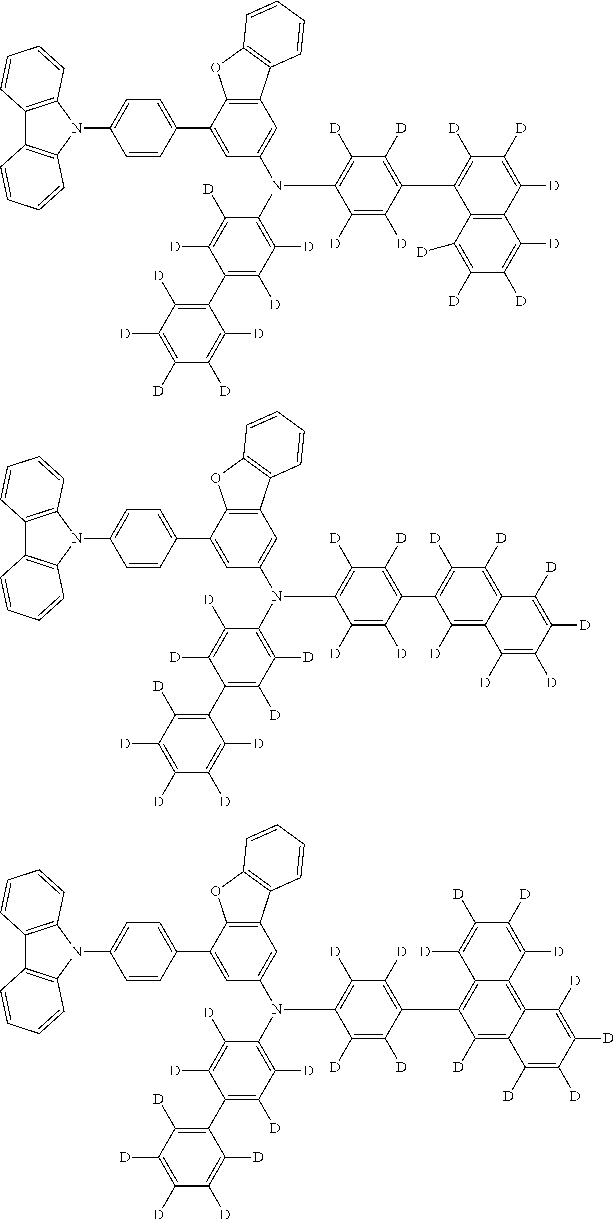

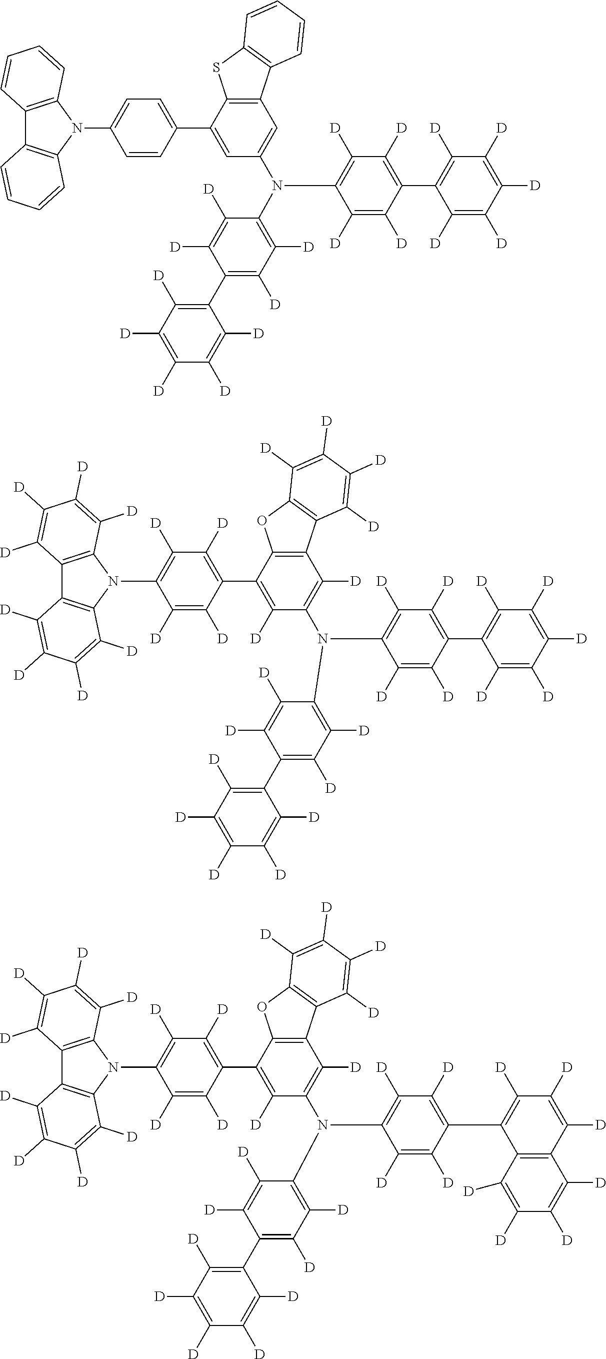

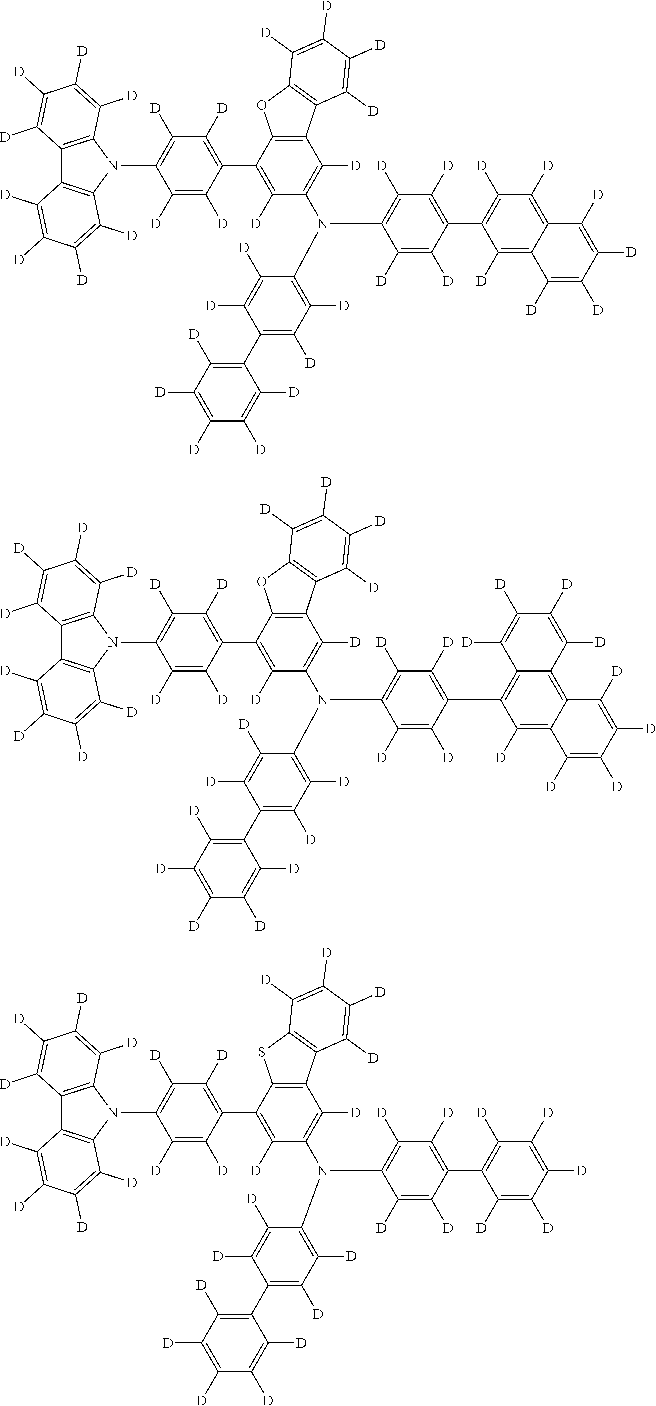

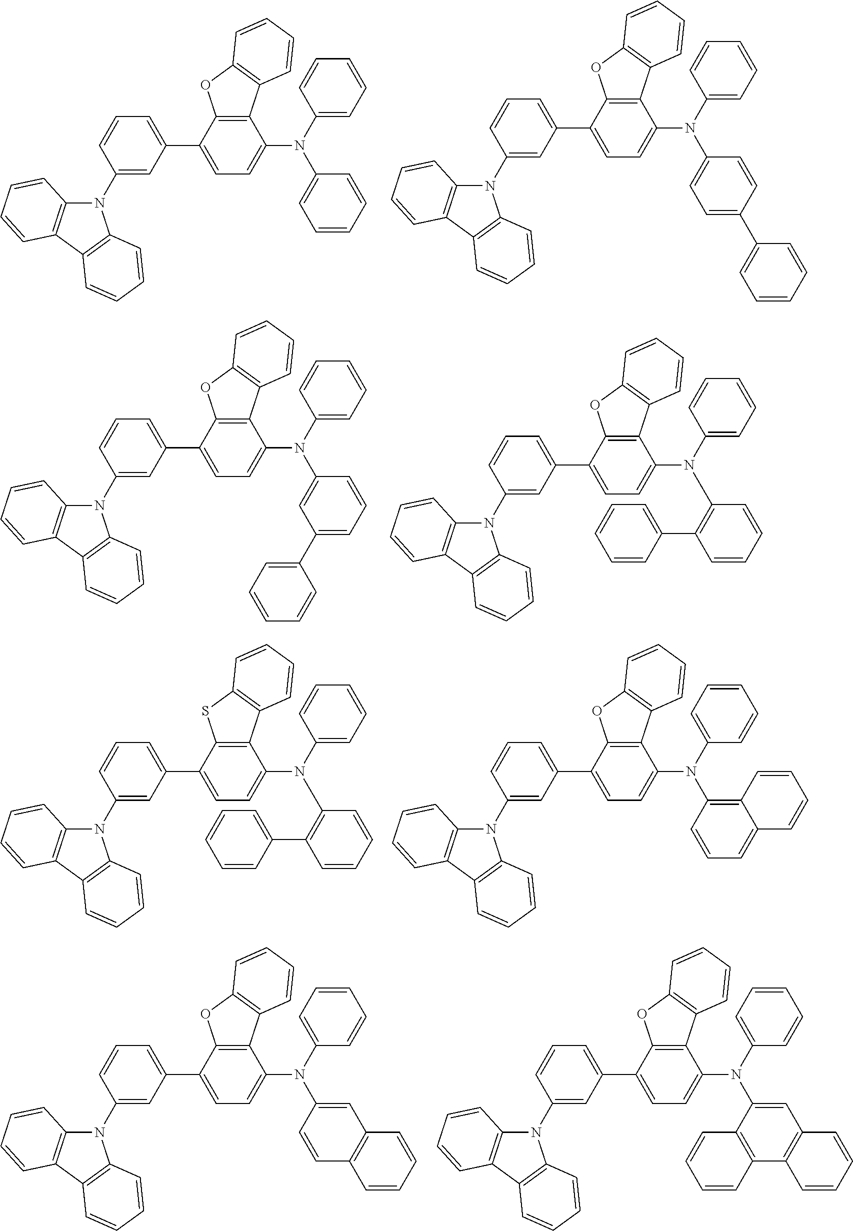

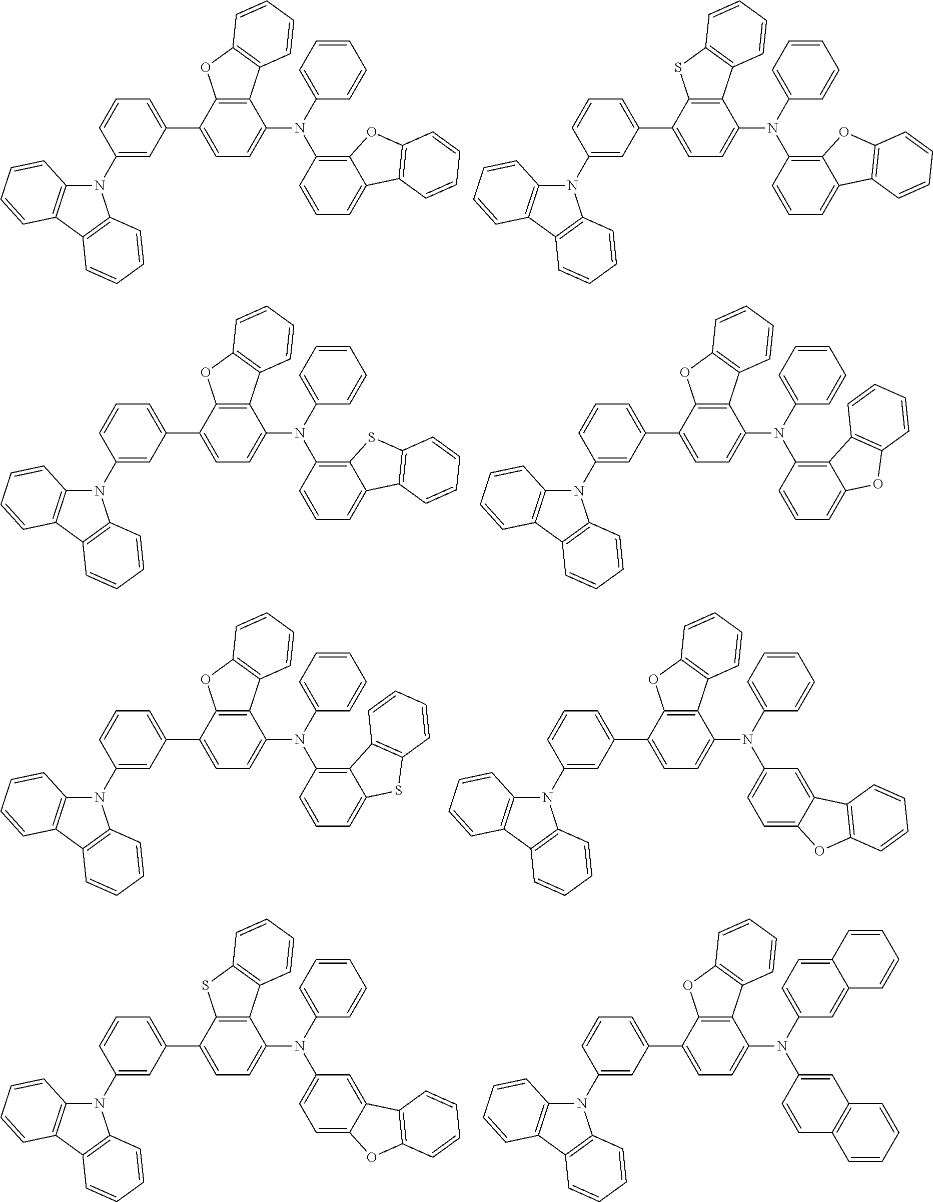

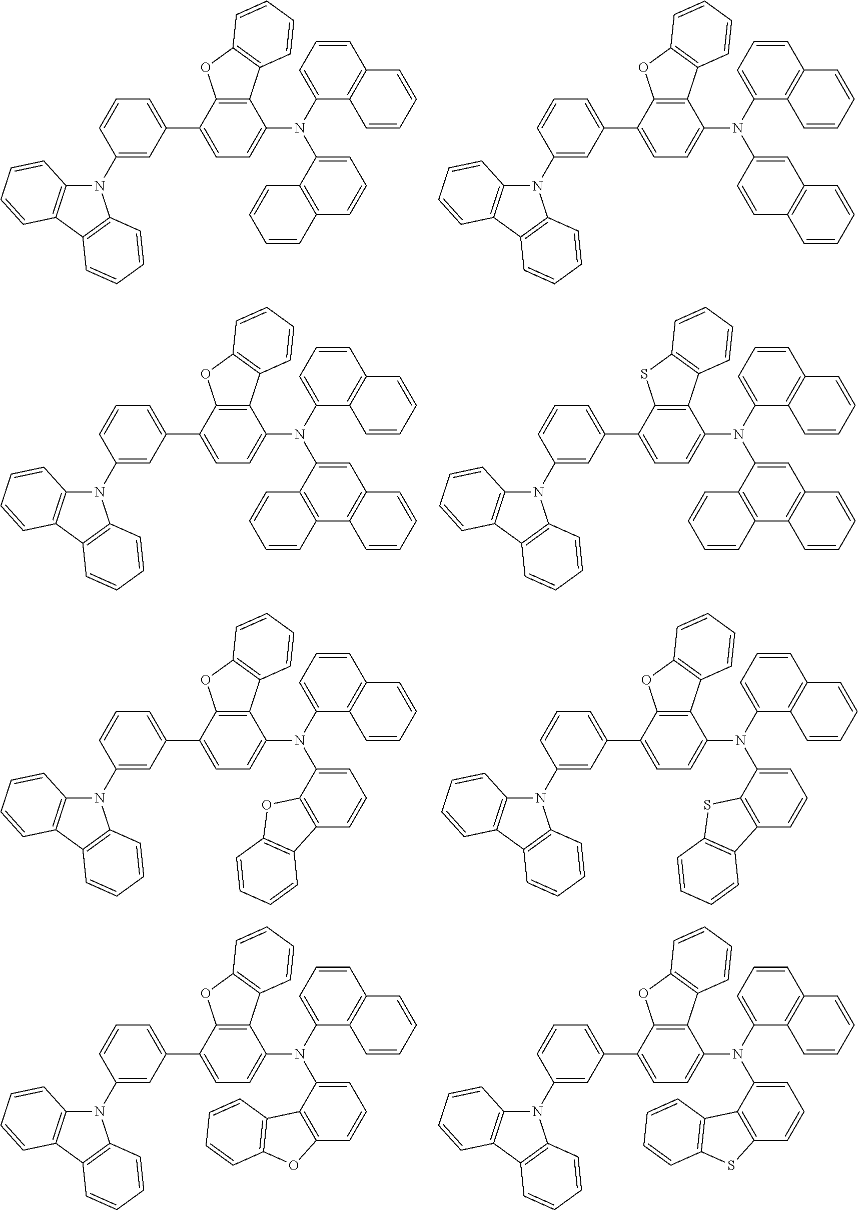

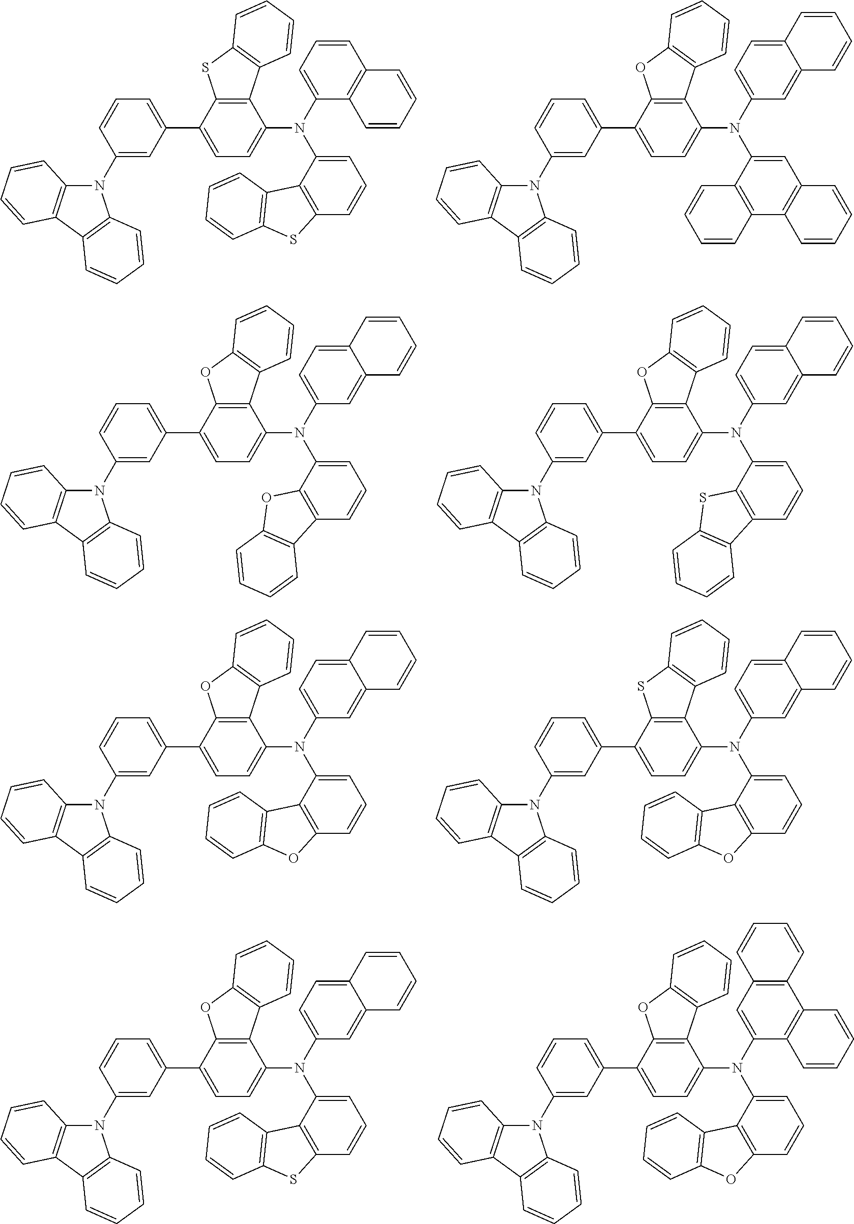

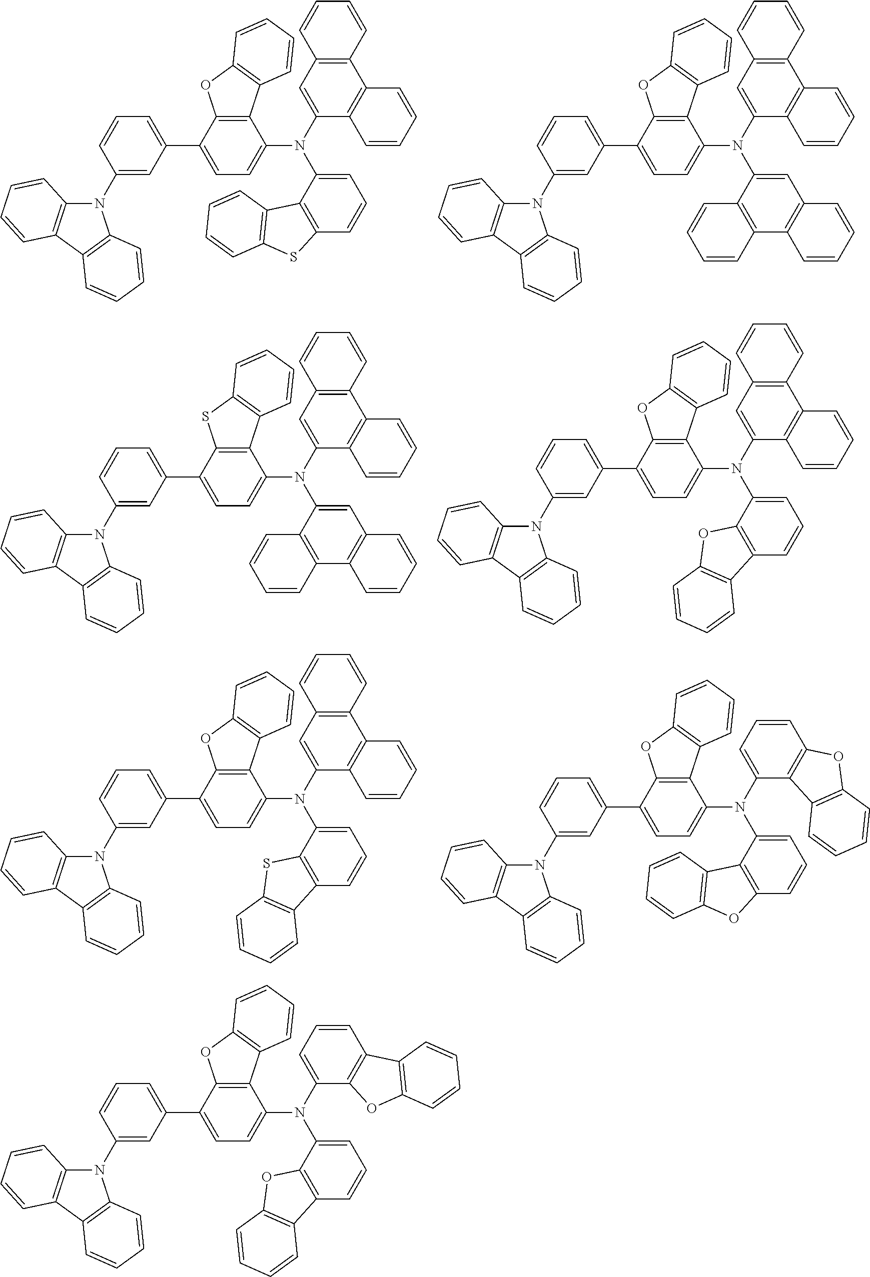

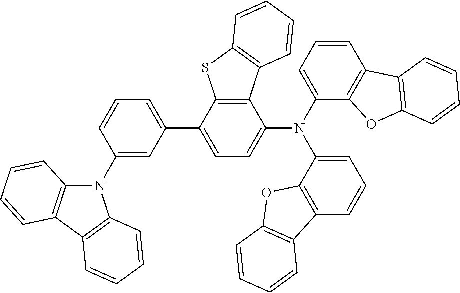 [SMILES: c1cc(-c2ccc(N(c3cccc4c3oc3ccccc34)c3cccc4c3oc3ccccc34)c3c2sc2ccccc23)cc(-n2c3ccccc3c3ccccc32)c1]

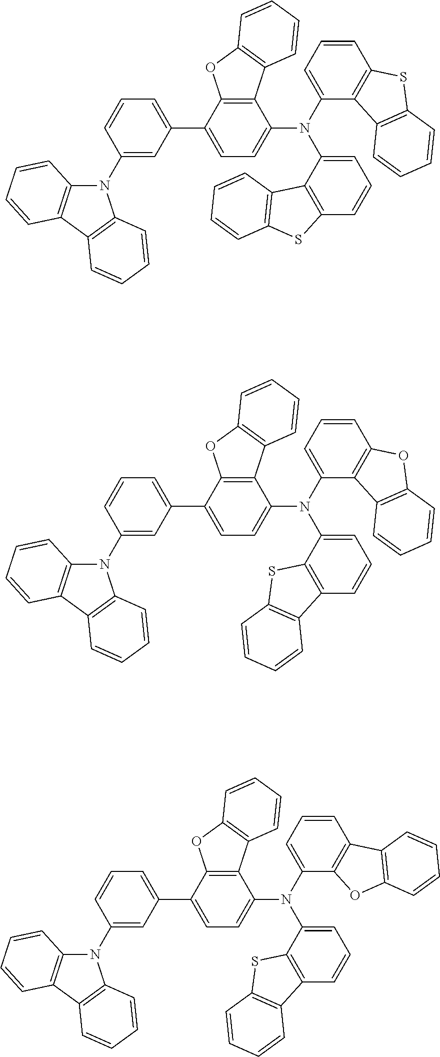 [SMILES: c1cc(-c2ccc(N(c3cccc4sc5ccccc5c34)c3cccc4sc5ccccc5c34)c3c2oc2ccccc23)cc(-n2c3ccccc3c3ccccc32)c1 ; c1cc(-c2ccc(N(c3cccc4c3sc3ccccc34)c3cccc4oc5ccccc5c34)c3c2oc2ccccc23)cc(-n2c3ccccc3c3ccccc32)c1 ; c1cc(-c2ccc(N(c3cccc4c3oc3ccccc34)c3cccc4c3sc3ccccc34)c3c2oc2ccccc23)cc(-n2c3ccccc3c3ccccc32)c1]

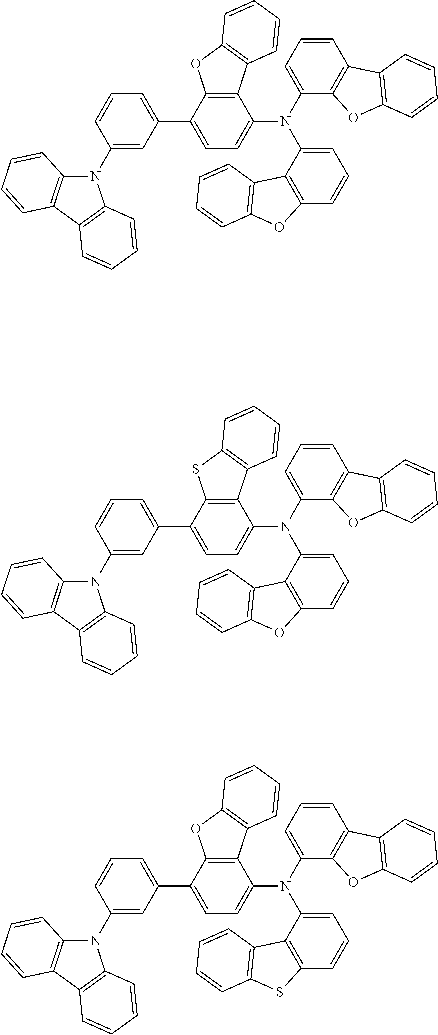 [SMILES: c1cc(-c2ccc(N(c3cccc4c3oc3ccccc34)c3cccc4oc5ccccc5c34)c3c2oc2ccccc23)cc(-n2c3ccccc3c3ccccc32)c1 ; c1cc(-c2ccc(N(c3cccc4c3oc3ccccc34)c3cccc4oc5ccccc5c34)c3c2sc2ccccc23)cc(-n2c3ccccc3c3ccccc32)c1 ; c1cc(-c2ccc(N(c3cccc4c3oc3ccccc34)c3cccc4sc5ccccc5c34)c3c2oc2ccccc23)cc(-n2c3ccccc3c3ccccc32)c1]

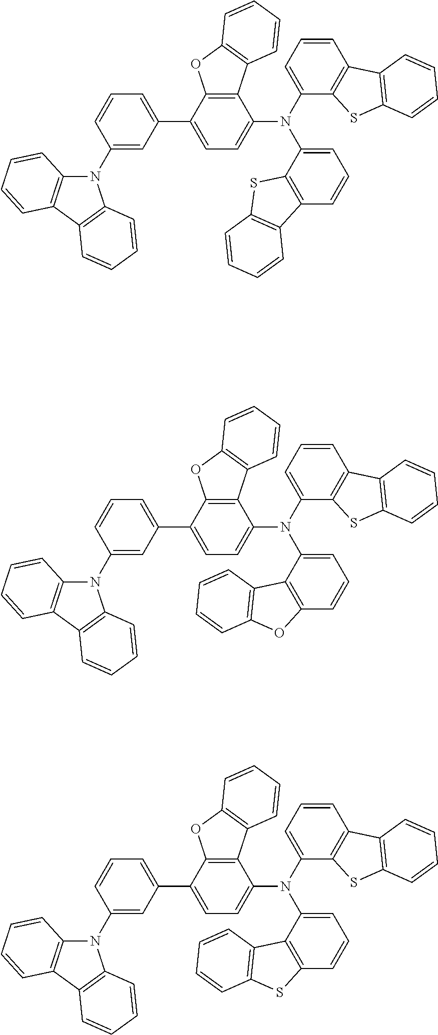 [SMILES: c1cc(-c2ccc(N(c3cccc4c3sc3ccccc34)c3cccc4c3sc3ccccc34)c3c2oc2ccccc23)cc(-n2c3ccccc3c3ccccc32)c1 ; c1cc(-c2ccc(N(c3cccc4c3sc3ccccc34)c3cccc4oc5ccccc5c34)c3c2oc2ccccc23)cc(-n2c3ccccc3c3ccccc32)c1 ; c1cc(-c2ccc(N(c3cccc4c3sc3ccccc34)c3cccc4sc5ccccc5c34)c3c2oc2ccccc23)cc(-n2c3ccccc3c3ccccc32)c1]

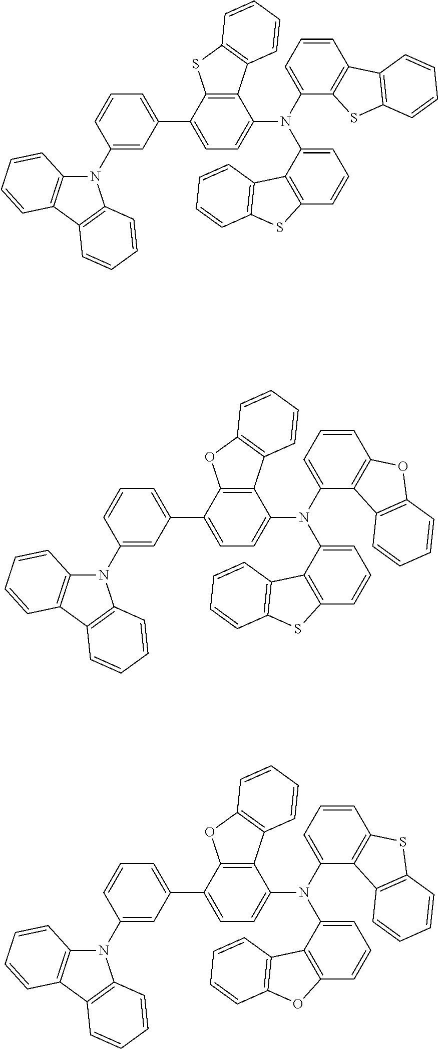 [SMILES: c1cc(-c2ccc(N(c3cccc4c3sc3ccccc34)c3cccc4sc5ccccc5c34)c3c2sc2ccccc23)cc(-n2c3ccccc3c3ccccc32)c1 ; c1cc(-c2ccc(N(c3cccc4oc5ccccc5c34)c3cccc4sc5ccccc5c34)c3c2oc2ccccc23)cc(-n2c3ccccc3c3ccccc32)c1 ; c1cc(-c2ccc(N(c3cccc4oc5ccccc5c34)c3cccc4sc5ccccc5c34)c3c2oc2ccccc23)cc(-n2c3ccccc3c3ccccc32)c1]

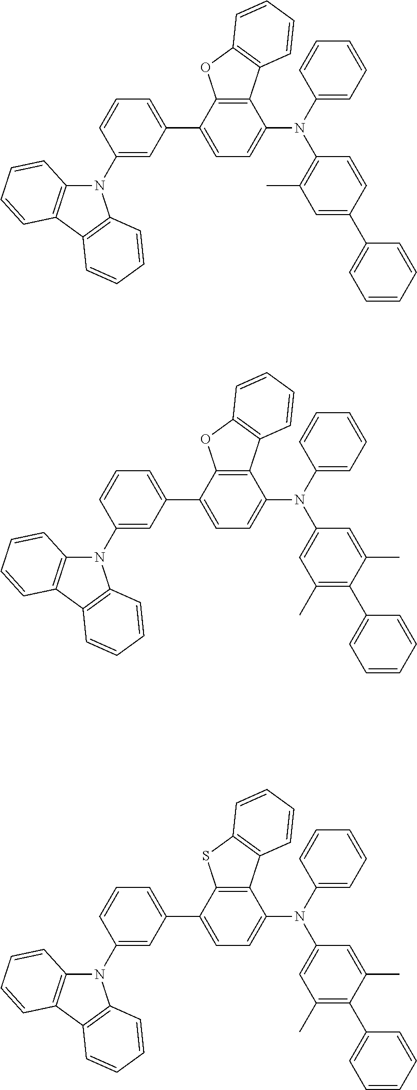 [SMILES: Cc1cc(-c2ccccc2)ccc1N(c1ccccc1)c1ccc(-c2cccc(-n3c4ccccc4c4ccccc43)c2)c2oc3ccccc3c12 ; Cc1cc(N(c2ccccc2)c2ccc(-c3cccc(-n4c5ccccc5c5ccccc54)c3)c3oc4ccccc4c23)cc(C)c1-c1ccccc1 ; Cc1cc(N(c2ccccc2)c2ccc(-c3cccc(-n4c5ccccc5c5ccccc54)c3)c3sc4ccccc4c23)cc(C)c1-c1ccccc1]

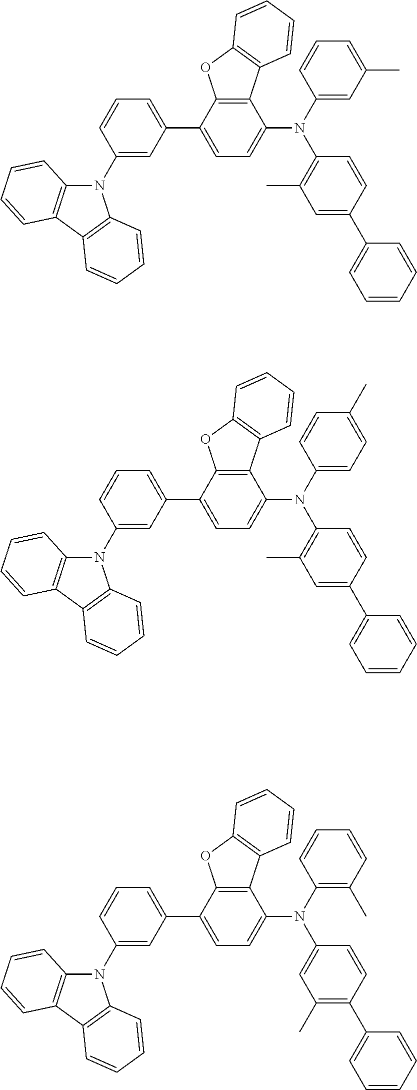 [SMILES: Cc1cccc(N(c2ccc(-c3ccccc3)cc2C)c2ccc(-c3cccc(-n4c5ccccc5c5ccccc54)c3)c3oc4ccccc4c23)c1 ; Cc1ccc(N(c2ccc(-c3ccccc3)cc2C)c2ccc(-c3cccc(-n4c5ccccc5c5ccccc54)c3)c3oc4ccccc4c23)cc1 ; Cc1cc(N(c2ccccc2C)c2ccc(-c3cccc(-n4c5ccccc5c5ccccc54)c3)c3oc4ccccc4c23)ccc1-c1ccccc1]

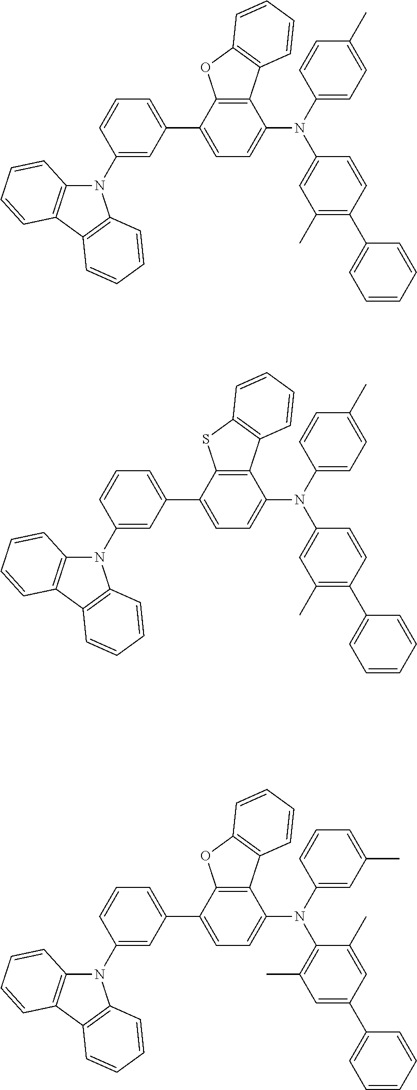 [SMILES: Cc1ccc(N(c2ccc(-c3ccccc3)c(C)c2)c2ccc(-c3cccc(-n4c5ccccc5c5ccccc54)c3)c3oc4ccccc4c23)cc1 ; Cc1ccc(N(c2ccc(-c3ccccc3)c(C)c2)c2ccc(-c3cccc(-n4c5ccccc5c5ccccc54)c3)c3sc4ccccc4c23)cc1 ; Cc1cccc(N(c2c(C)cc(-c3ccccc3)cc2C)c2ccc(-c3cccc(-n4c5ccccc5c5ccccc54)c3)c3oc4ccccc4c23)c1]

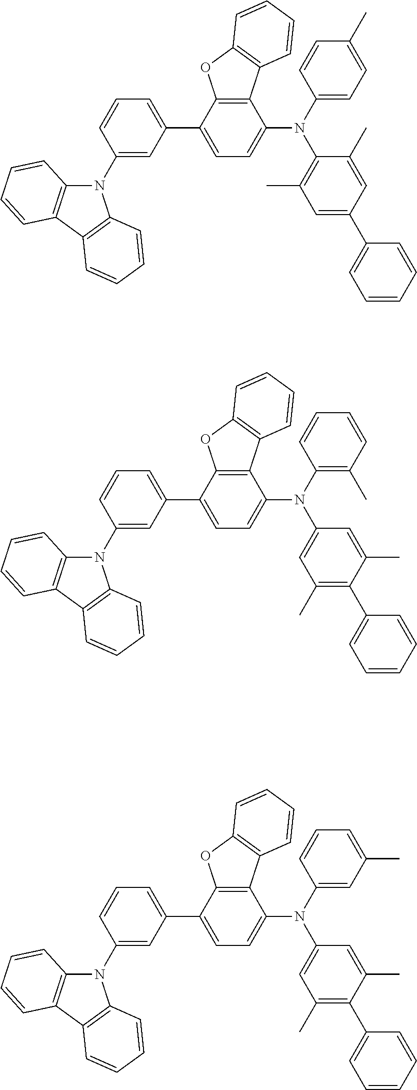 [SMILES: Cc1ccc(N(c2c(C)cc(-c3ccccc3)cc2C)c2ccc(-c3cccc(-n4c5ccccc5c5ccccc54)c3)c3oc4ccccc4c23)cc1 ; Cc1ccccc1N(c1cc(C)c(-c2ccccc2)c(C)c1)c1ccc(-c2cccc(-n3c4ccccc4c4ccccc43)c2)c2oc3ccccc3c12 ; Cc1cccc(N(c2cc(C)c(-c3ccccc3)c(C)c2)c2ccc(-c3cccc(-n4c5ccccc5c5ccccc54)c3)c3oc4ccccc4c23)c1]

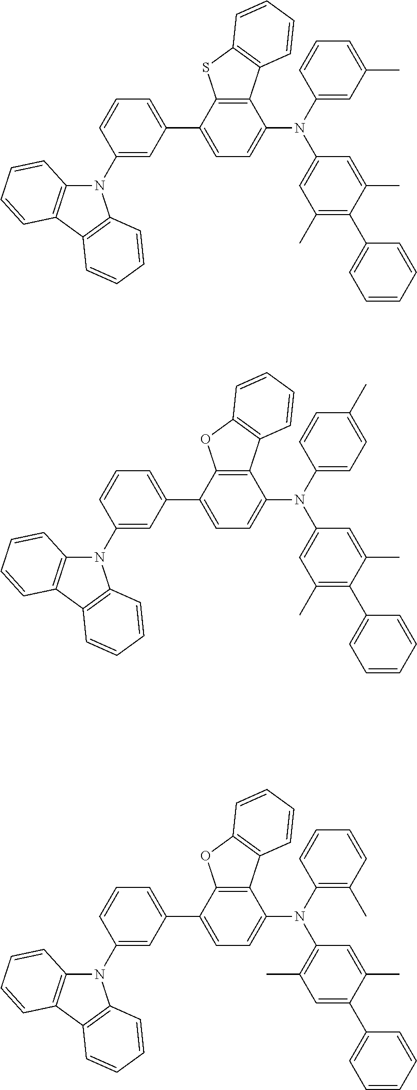 [SMILES: Cc1cccc(N(c2cc(C)c(-c3ccccc3)c(C)c2)c2ccc(-c3cccc(-n4c5ccccc5c5ccccc54)c3)c3sc4ccccc4c23)c1 ; Cc1ccc(N(c2cc(C)c(-c3ccccc3)c(C)c2)c2ccc(-c3cccc(-n4c5ccccc5c5ccccc54)c3)c3oc4ccccc4c23)cc1 ; Cc1cc(N(c2ccccc2C)c2ccc(-c3cccc(-n4c5ccccc5c5ccccc54)c3)c3oc4ccccc4c23)c(C)cc1-c1ccccc1]

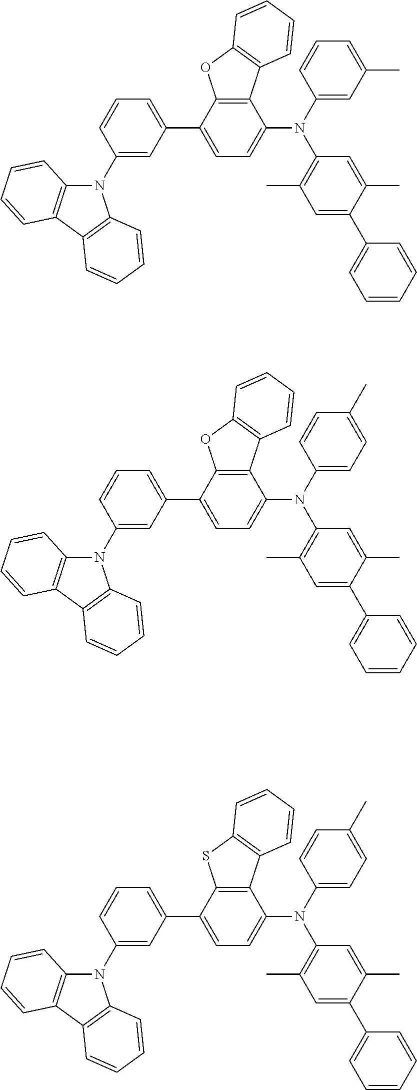 [SMILES: Cc1cccc(N(c2cc(C)c(-c3ccccc3)cc2C)c2ccc(-c3cccc(-n4c5ccccc5c5ccccc54)c3)c3oc4ccccc4c23)c1 ; Cc1ccc(N(c2cc(C)c(-c3ccccc3)cc2C)c2ccc(-c3cccc(-n4c5ccccc5c5ccccc54)c3)c3oc4ccccc4c23)cc1 ; Cc1ccc(N(c2cc(C)c(-c3ccccc3)cc2C)c2ccc(-c3cccc(-n4c5ccccc5c5ccccc54)c3)c3sc4ccccc4c23)cc1]

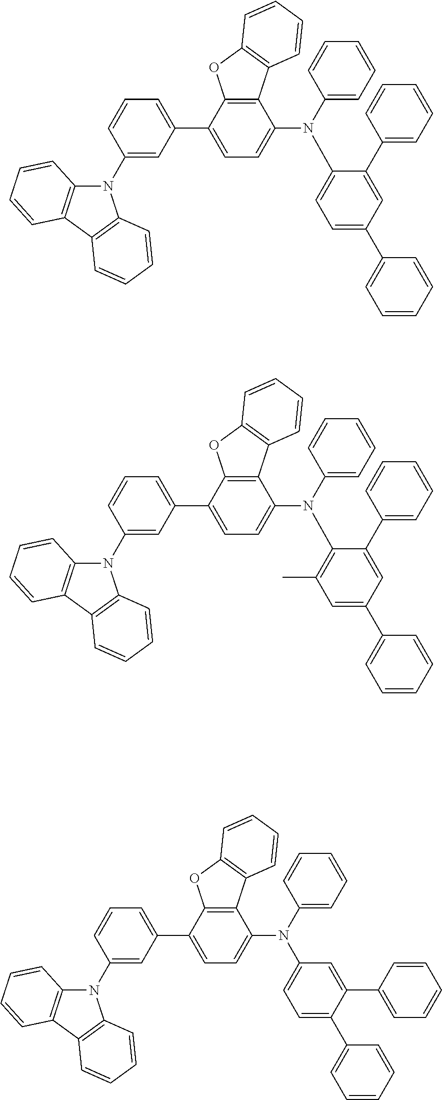 [SMILES: c1ccc(-c2ccc(N(c3ccccc3)c3ccc(-c4cccc(-n5c6ccccc6c6ccccc65)c4)c4oc5ccccc5c34)c(-c3ccccc3)c2)cc1 ; Cc1cc(-c2ccccc2)cc(-c2ccccc2)c1N(c1ccccc1)c1ccc(-c2cccc(-n3c4ccccc4c4ccccc43)c2)c2oc3ccccc3c12 ; c1ccc(-c2ccc(N(c3ccccc3)c3ccc(-c4cccc(-n5c6ccccc6c6ccccc65)c4)c4oc5ccccc5c34)cc2-c2ccccc2)cc1]

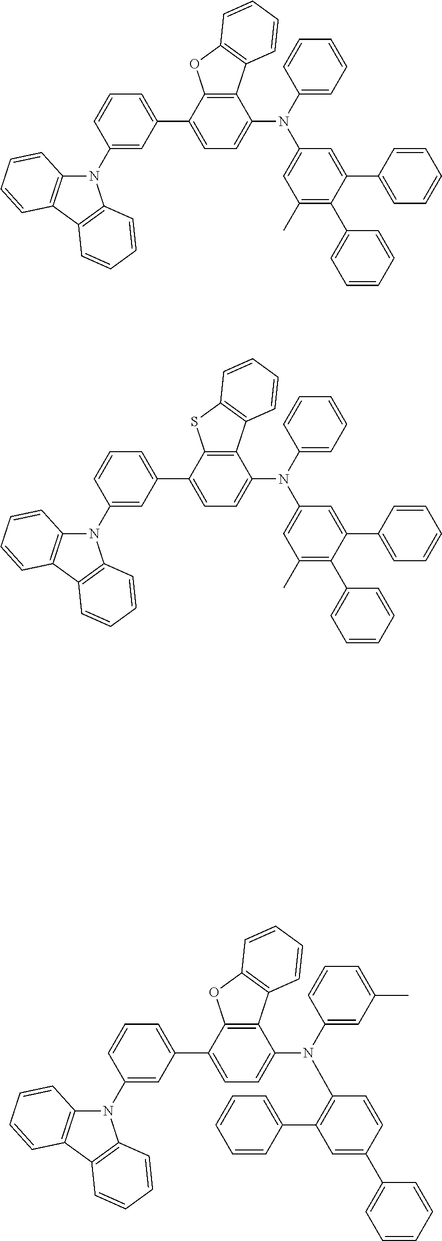 [SMILES: Cc1cc(N(c2ccccc2)c2ccc(-c3cccc(-n4c5ccccc5c5ccccc54)c3)c3oc4ccccc4c23)cc(-c2ccccc2)c1-c1ccccc1 ; Cc1cc(N(c2ccccc2)c2ccc(-c3cccc(-n4c5ccccc5c5ccccc54)c3)c3sc4ccccc4c23)cc(-c2ccccc2)c1-c1ccccc1 ; Cc1cccc(N(c2ccc(-c3ccccc3)cc2-c2ccccc2)c2ccc(-c3cccc(-n4c5ccccc5c5ccccc54)c3)c3oc4ccccc4c23)c1]

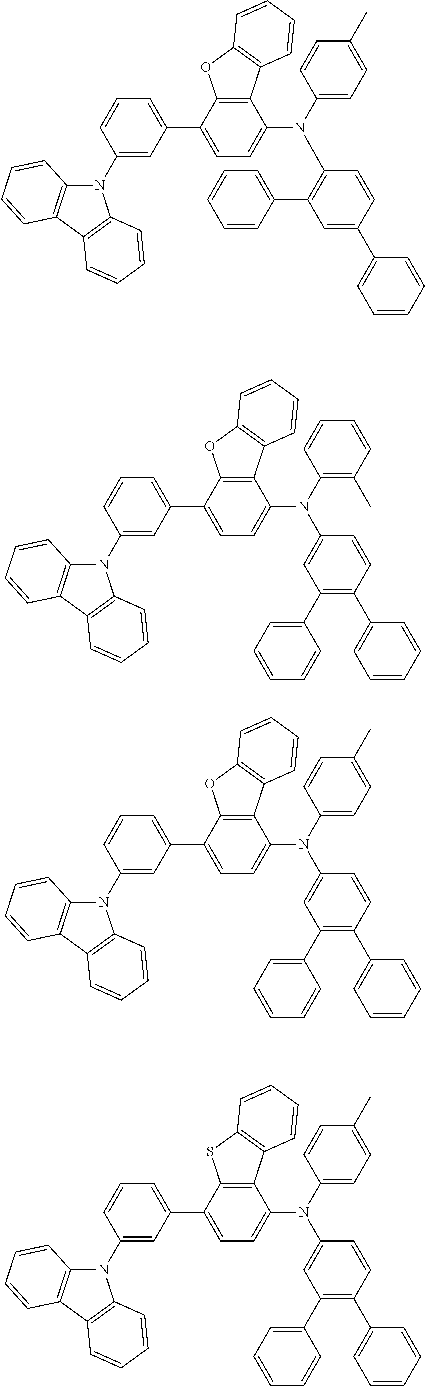

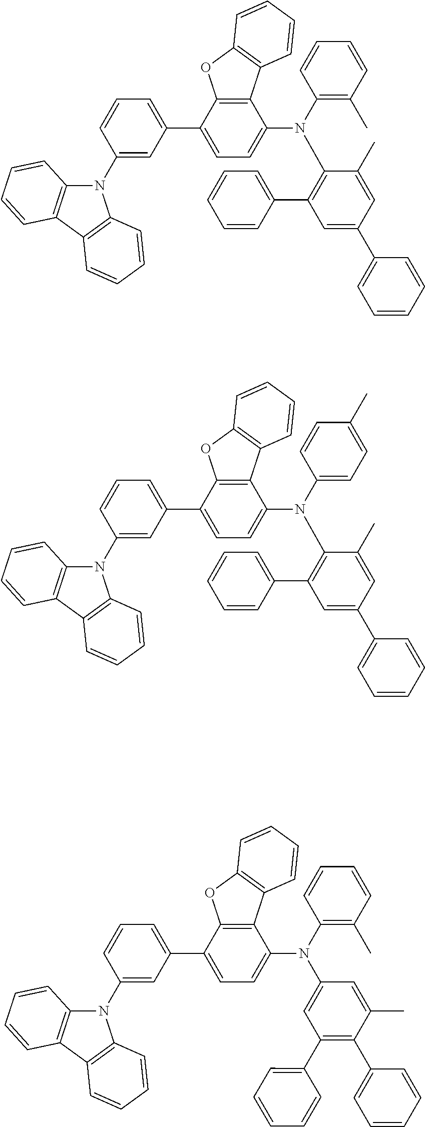 [SMILES: Cc1ccccc1N(c1c(C)cc(-c2ccccc2)cc1-c1ccccc1)c1ccc(-c2cccc(-n3c4ccccc4c4ccccc43)c2)c2oc3ccccc3c12 ; Cc1ccc(N(c2c(C)cc(-c3ccccc3)cc2-c2ccccc2)c2ccc(-c3cccc(-n4c5ccccc5c5ccccc54)c3)c3oc4ccccc4c23)cc1 ; Cc1ccccc1N(c1cc(C)c(-c2ccccc2)c(-c2ccccc2)c1)c1ccc(-c2cccc(-n3c4ccccc4c4ccccc43)c2)c2oc3ccccc3c12]

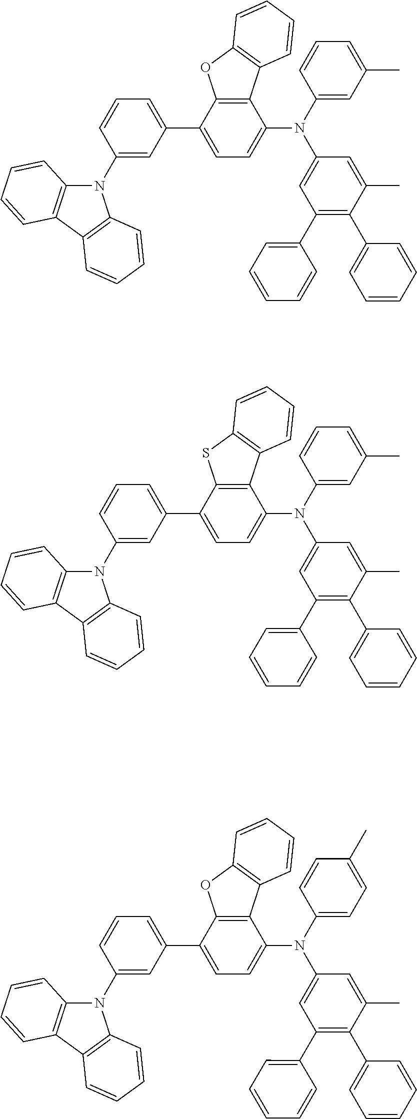 [SMILES: Cc1cccc(N(c2cc(C)c(-c3ccccc3)c(-c3ccccc3)c2)c2ccc(-c3cccc(-n4c5ccccc5c5ccccc54)c3)c3oc4ccccc4c23)c1 ; Cc1cccc(N(c2cc(C)c(-c3ccccc3)c(-c3ccccc3)c2)c2ccc(-c3cccc(-n4c5ccccc5c5ccccc54)c3)c3sc4ccccc4c23)c1 ; Cc1ccc(N(c2cc(C)c(-c3ccccc3)c(-c3ccccc3)c2)c2ccc(-c3cccc(-n4c5ccccc5c5ccccc54)c3)c3oc4ccccc4c23)cc1]

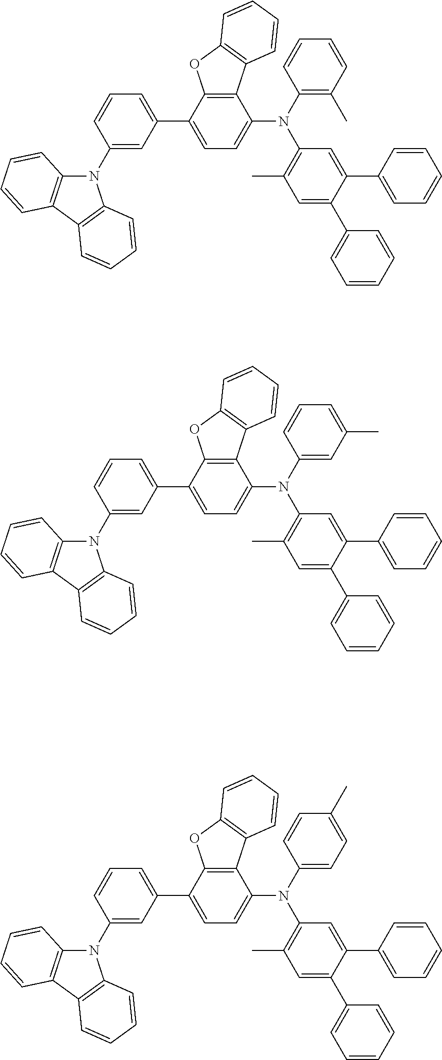 [SMILES: Cc1ccccc1N(c1cc(-c2ccccc2)c(-c2ccccc2)cc1C)c1ccc(-c2cccc(-n3c4ccccc4c4ccccc43)c2)c2oc3ccccc3c12 ; Cc1cccc(N(c2cc(-c3ccccc3)c(-c3ccccc3)cc2C)c2ccc(-c3cccc(-n4c5ccccc5c5ccccc54)c3)c3oc4ccccc4c23)c1 ; Cc1ccc(N(c2cc(-c3ccccc3)c(-c3ccccc3)cc2C)c2ccc(-c3cccc(-n4c5ccccc5c5ccccc54)c3)c3oc4ccccc4c23)cc1]

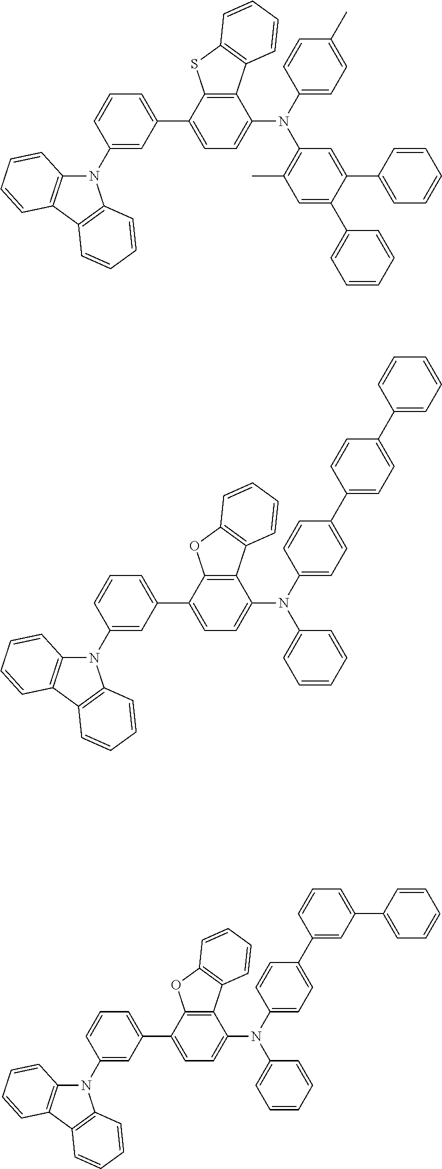 [SMILES: Cc1ccc(N(c2cc(-c3ccccc3)c(-c3ccccc3)cc2C)c2ccc(-c3cccc(-n4c5ccccc5c5ccccc54)c3)c3sc4ccccc4c23)cc1 ; c1ccc(-c2ccc(-c3ccc(N(c4ccccc4)c4ccc(-c5cccc(-n6c7ccccc7c7ccccc76)c5)c5oc6ccccc6c45)cc3)cc2)cc1 ; c1ccc(-c2cccc(-c3ccc(N(c4ccccc4)c4ccc(-c5cccc(-n6c7ccccc7c7ccccc76)c5)c5oc6ccccc6c45)cc3)c2)cc1]

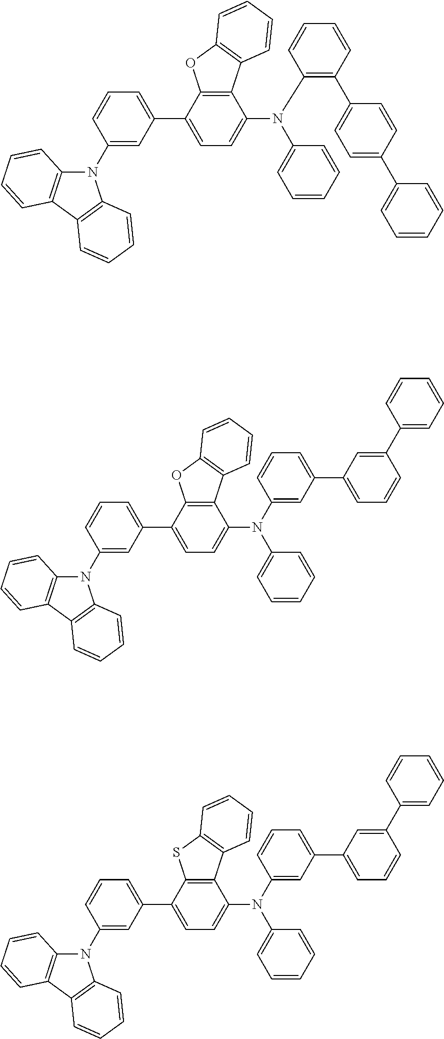 [SMILES: c1ccc(-c2ccc(-c3ccccc3N(c3ccccc3)c3ccc(-c4cccc(-n5c6ccccc6c6ccccc65)c4)c4oc5ccccc5c34)cc2)cc1 ; c1ccc(-c2cccc(-c3cccc(N(c4ccccc4)c4ccc(-c5cccc(-n6c7ccccc7c7ccccc76)c5)c5oc6ccccc6c45)c3)c2)cc1 ; c1ccc(-c2cccc(-c3cccc(N(c4ccccc4)c4ccc(-c5cccc(-n6c7ccccc7c7ccccc76)c5)c5sc6ccccc6c45)c3)c2)cc1]

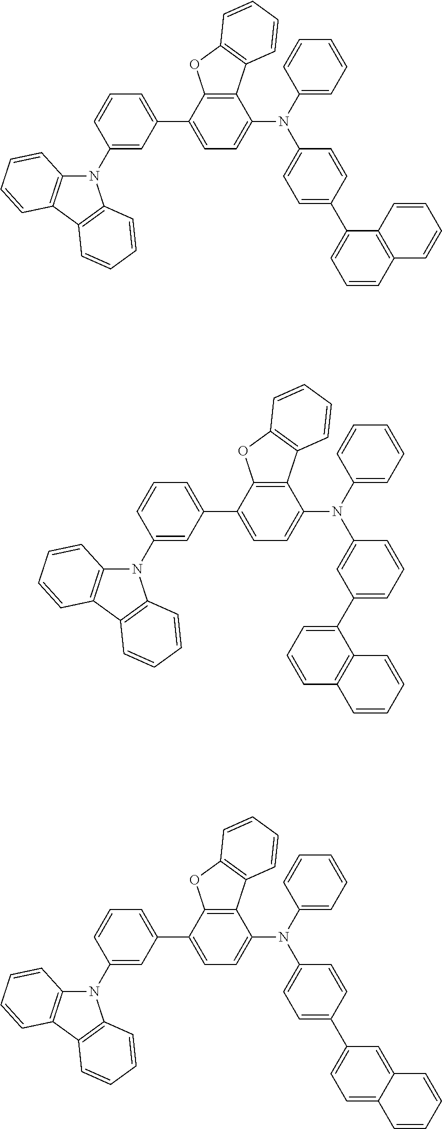 [SMILES: c1ccc(N(c2ccc(-c3cccc4ccccc34)cc2)c2ccc(-c3cccc(-n4c5ccccc5c5ccccc54)c3)c3oc4ccccc4c23)cc1 ; c1ccc(N(c2cccc(-c3cccc4ccccc34)c2)c2ccc(-c3cccc(-n4c5ccccc5c5ccccc54)c3)c3oc4ccccc4c23)cc1 ; c1ccc(N(c2ccc(-c3ccc4ccccc4c3)cc2)c2ccc(-c3cccc(-n4c5ccccc5c5ccccc54)c3)c3oc4ccccc4c23)cc1]

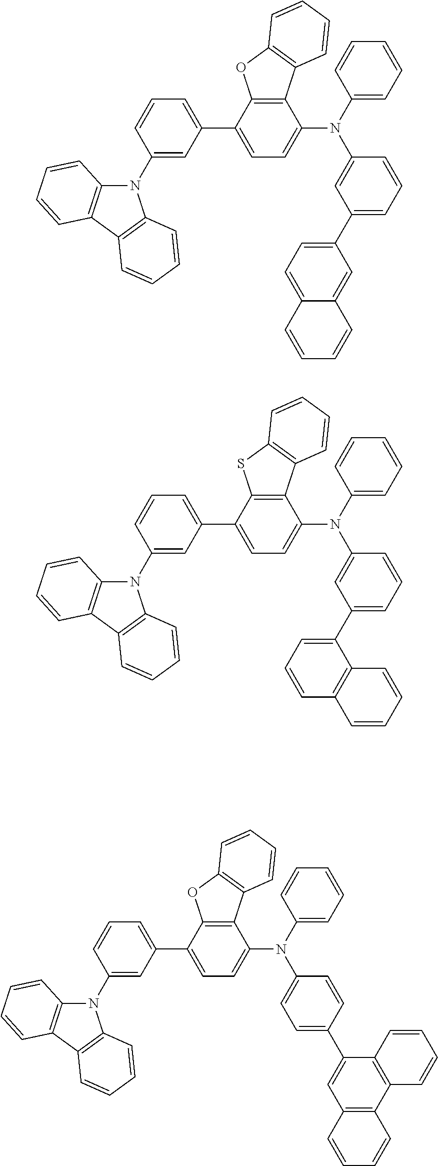

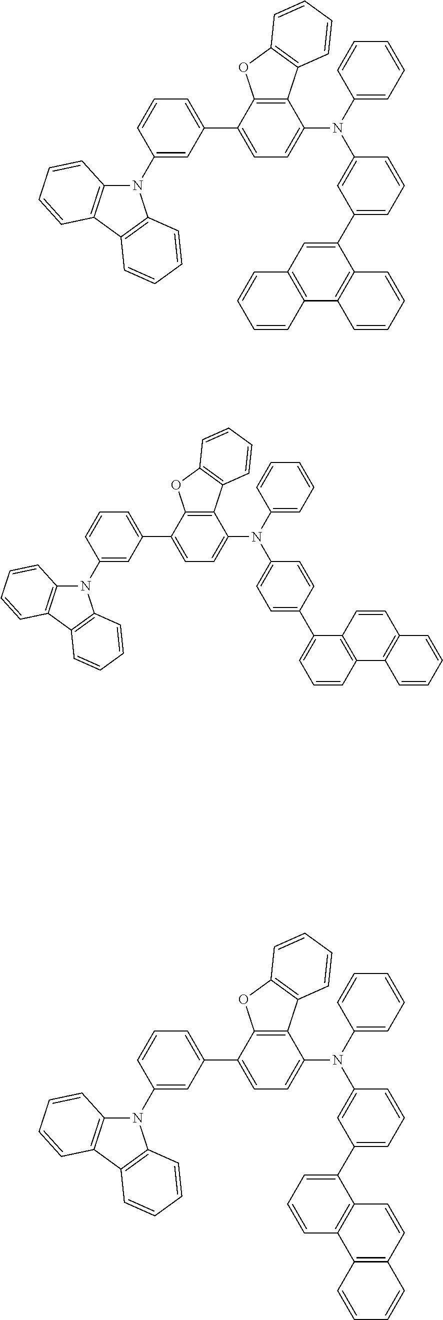 [SMILES: c1ccc(N(c2cccc(-c3cc4ccccc4c4ccccc34)c2)c2ccc(-c3cccc(-n4c5ccccc5c5ccccc54)c3)c3oc4ccccc4c23)cc1 ; c1ccc(N(c2ccc(-c3cccc4c3ccc3ccccc34)cc2)c2ccc(-c3cccc(-n4c5ccccc5c5ccccc54)c3)c3oc4ccccc4c23)cc1 ; c1ccc(N(c2cccc(-c3cccc4c3ccc3ccccc34)c2)c2ccc(-c3cccc(-n4c5ccccc5c5ccccc54)c3)c3oc4ccccc4c23)cc1]

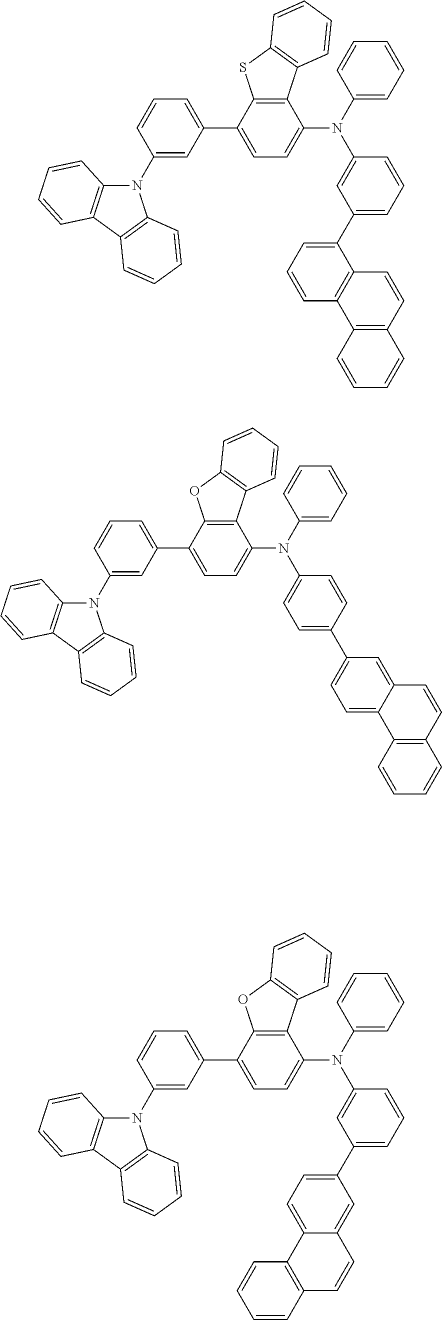 [SMILES: c1ccc(N(c2cccc(-c3cccc4c3ccc3ccccc34)c2)c2ccc(-c3cccc(-n4c5ccccc5c5ccccc54)c3)c3sc4ccccc4c23)cc1 ; c1ccc(N(c2ccc(-c3ccc4c(ccc5ccccc54)c3)cc2)c2ccc(-c3cccc(-n4c5ccccc5c5ccccc54)c3)c3oc4ccccc4c23)cc1 ; c1ccc(N(c2cccc(-c3ccc4c(ccc5ccccc54)c3)c2)c2ccc(-c3cccc(-n4c5ccccc5c5ccccc54)c3)c3oc4ccccc4c23)cc1]

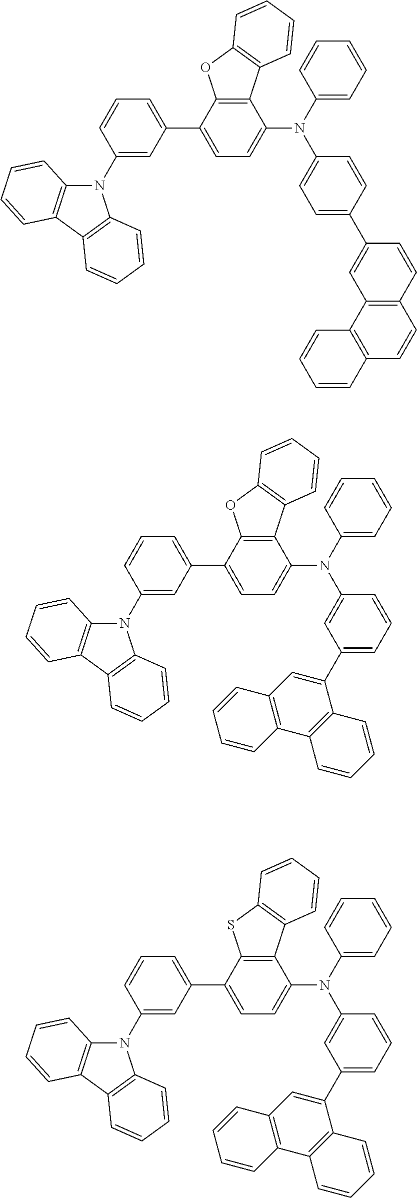 [SMILES: c1ccc(N(c2ccc(-c3ccc4ccc5ccccc5c4c3)cc2)c2ccc(-c3cccc(-n4c5ccccc5c5ccccc54)c3)c3oc4ccccc4c23)cc1 ; c1ccc(N(c2cccc(-c3cc4ccccc4c4ccccc34)c2)c2ccc(-c3cccc(-n4c5ccccc5c5ccccc54)c3)c3oc4ccccc4c23)cc1 ; c1ccc(N(c2cccc(-c3cc4ccccc4c4ccccc34)c2)c2ccc(-c3cccc(-n4c5ccccc5c5ccccc54)c3)c3sc4ccccc4c23)cc1]

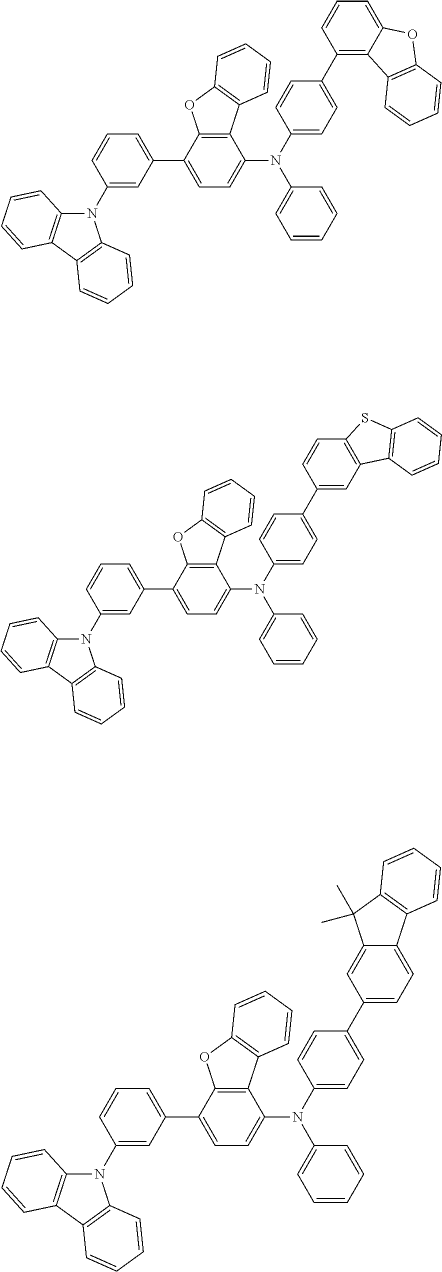 [SMILES: c1ccc(N(c2ccc(-c3cccc4oc5ccccc5c34)cc2)c2ccc(-c3cccc(-n4c5ccccc5c5ccccc54)c3)c3oc4ccccc4c23)cc1 ; c1ccc(N(c2ccc(-c3ccc4sc5ccccc5c4c3)cc2)c2ccc(-c3cccc(-n4c5ccccc5c5ccccc54)c3)c3oc4ccccc4c23)cc1 ; CC1(C)c2ccccc2-c2ccc(-c3ccc(N(c4ccccc4)c4ccc(-c5cccc(-n6c7ccccc7c7ccccc76)c5)c5oc6ccccc6c45)cc3)cc21]

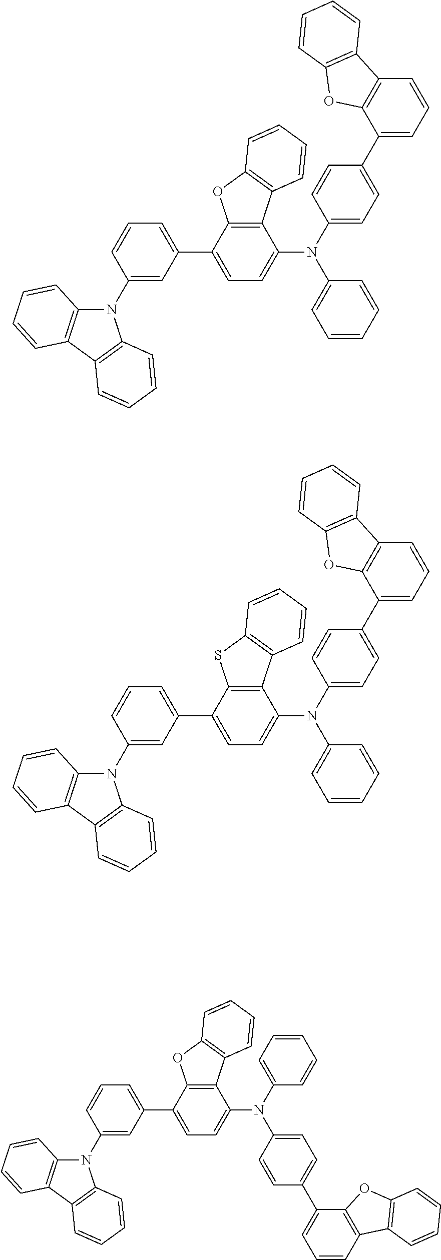 [SMILES: c1ccc(N(c2ccc(-c3cccc4c3oc3ccccc34)cc2)c2ccc(-c3cccc(-n4c5ccccc5c5ccccc54)c3)c3oc4ccccc4c23)cc1 ; c1ccc(N(c2ccc(-c3cccc4c3oc3ccccc34)cc2)c2ccc(-c3cccc(-n4c5ccccc5c5ccccc54)c3)c3sc4ccccc4c23)cc1 ; c1ccc(N(c2ccc(-c3cccc4c3oc3ccccc34)cc2)c2ccc(-c3cccc(-n4c5ccccc5c5ccccc54)c3)c3oc4ccccc4c23)cc1]

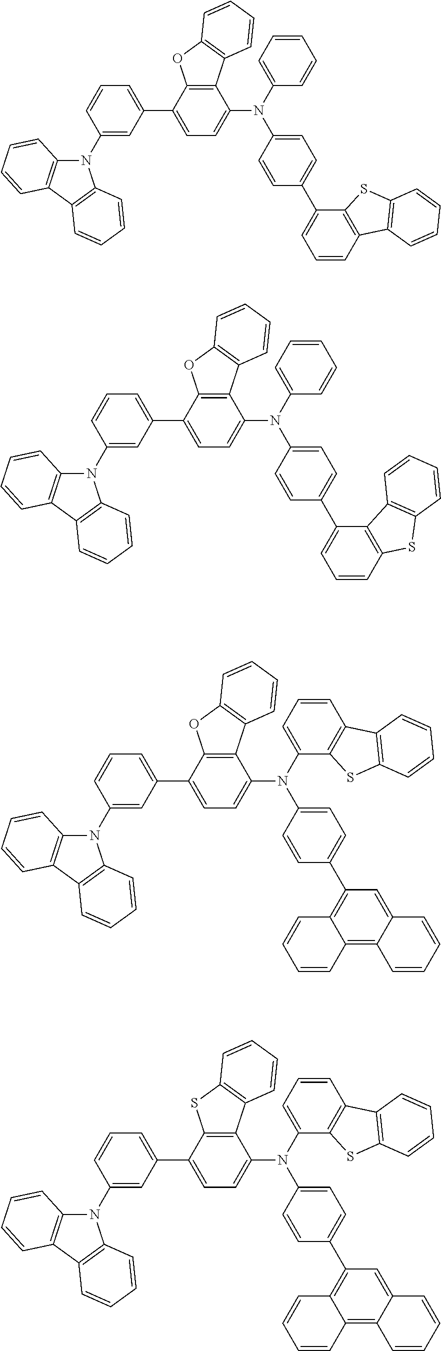 [SMILES: c1ccc(N(c2ccc(-c3cccc4c3sc3ccccc34)cc2)c2ccc(-c3cccc(-n4c5ccccc5c5ccccc54)c3)c3oc4ccccc4c23)cc1 ; c1ccc(N(c2ccc(-c3cccc4sc5ccccc5c34)cc2)c2ccc(-c3cccc(-n4c5ccccc5c5ccccc54)c3)c3oc4ccccc4c23)cc1 ; c1cc(-c2ccc(N(c3ccc(-c4cc5ccccc5c5ccccc45)cc3)c3cccc4c3sc3ccccc34)c3c2oc2ccccc23)cc(-n2c3ccccc3c3ccccc32)c1 ; c1cc(-c2ccc(N(c3ccc(-c4cc5ccccc5c5ccccc45)cc3)c3cccc4c3sc3ccccc34)c3c2sc2ccccc23)cc(-n2c3ccccc3c3ccccc32)c1]

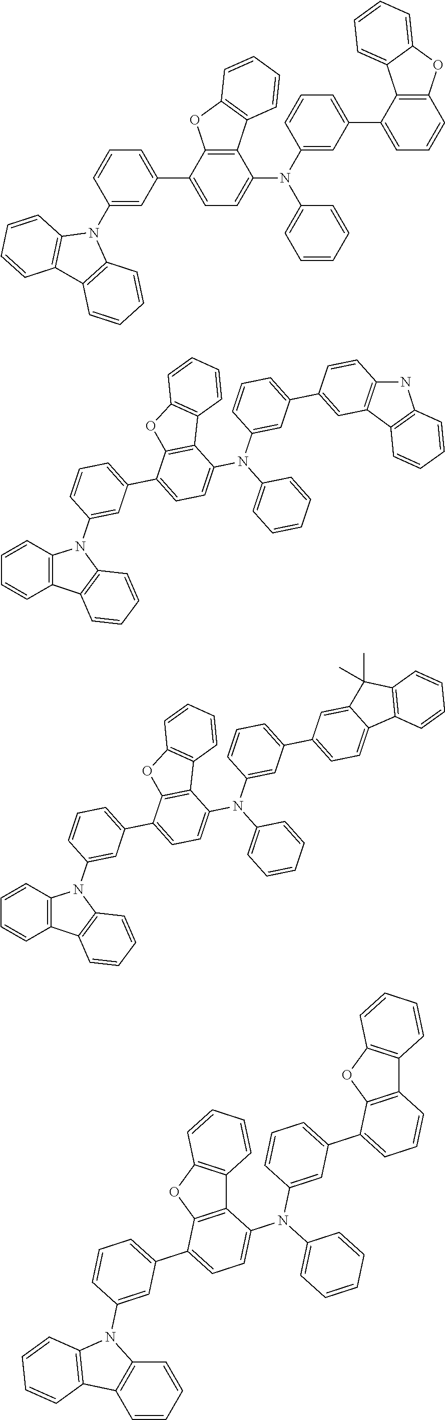 [SMILES: c1ccc(N(c2cccc(-c3cccc4oc5ccccc5c34)c2)c2ccc(-c3cccc(-n4c5ccccc5c5ccccc54)c3)c3oc4ccccc4c23)cc1 ; c1ccc(N(c2cccc(-c3ccc4[nH]c5ccccc5c4c3)c2)c2ccc(-c3cccc(-n4c5ccccc5c5ccccc54)c3)c3oc4ccccc4c23)cc1 ; CC1(C)c2ccccc2-c2ccc(-c3cccc(N(c4ccccc4)c4ccc(-c5cccc(-n6c7ccccc7c7ccccc76)c5)c5oc6ccccc6c45)c3)cc21 ; c1ccc(N(c2cccc(-c3cccc4c3oc3ccccc34)c2)c2ccc(-c3cccc(-n4c5ccccc5c5ccccc54)c3)c3oc4ccccc4c23)cc1]

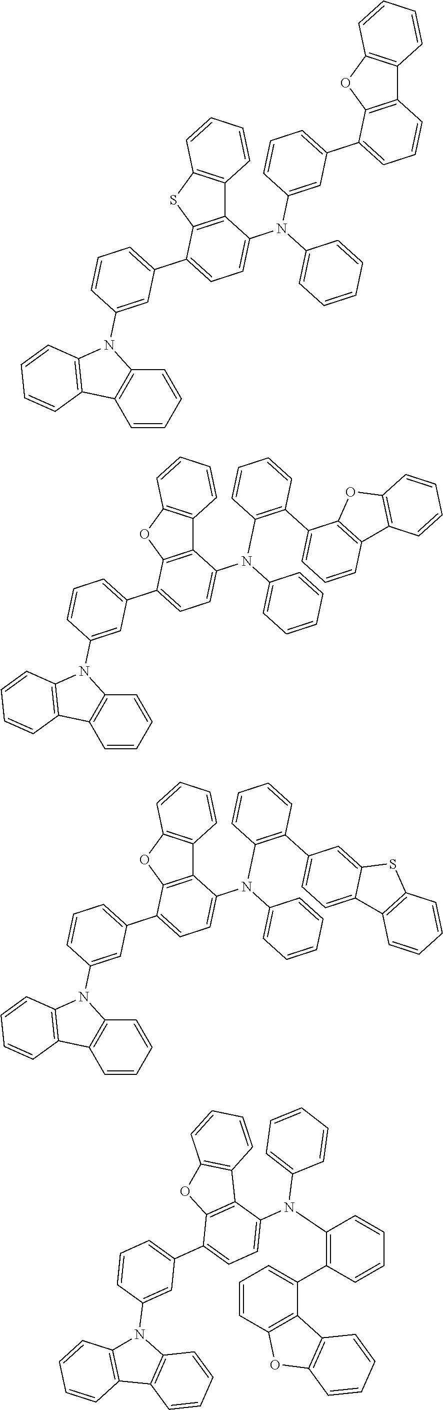

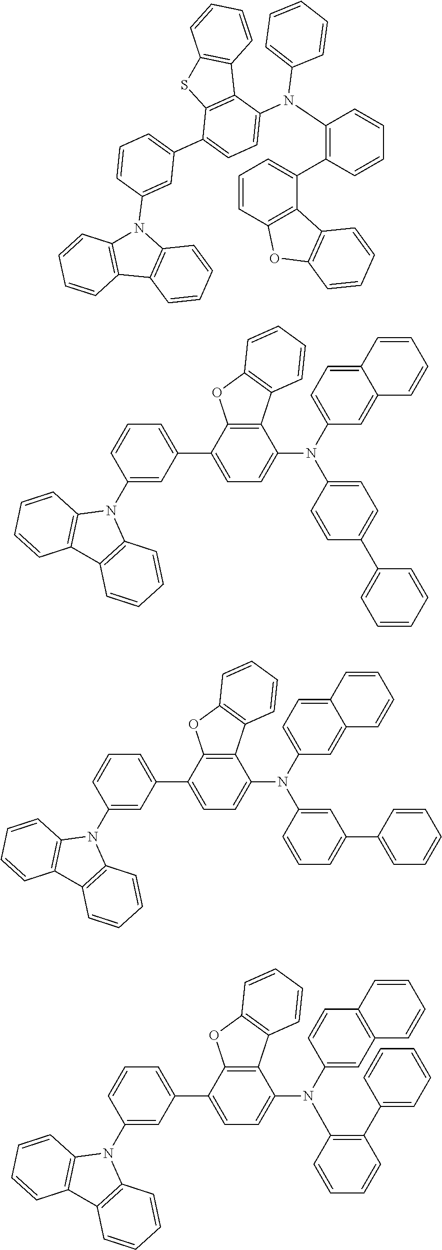

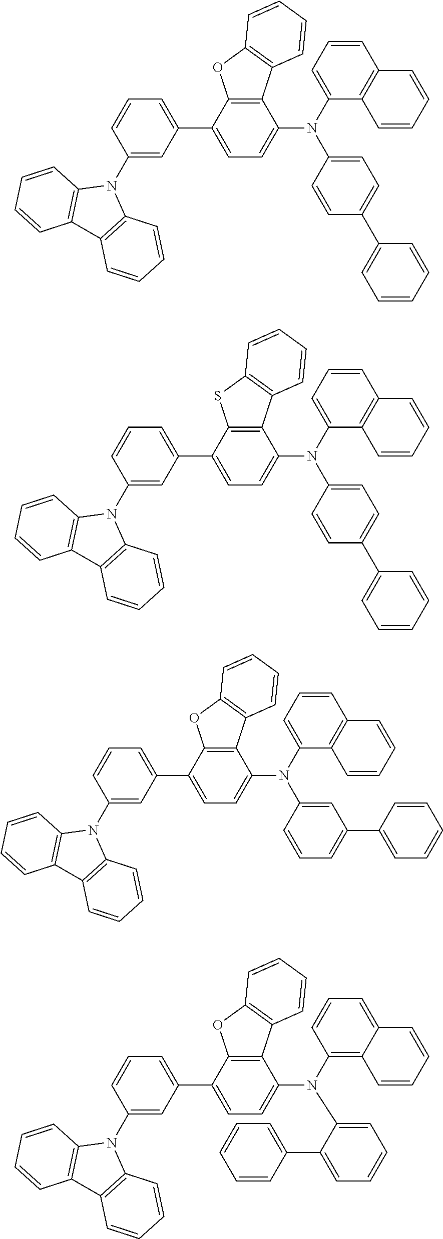 [SMILES: c1ccc(-c2ccc(N(c3cccc4ccccc34)c3ccc(-c4cccc(-n5c6ccccc6c6ccccc65)c4)c4oc5ccccc5c34)cc2)cc1 ; c1ccc(-c2ccc(N(c3cccc4ccccc34)c3ccc(-c4cccc(-n5c6ccccc6c6ccccc65)c4)c4sc5ccccc5c34)cc2)cc1 ; c1ccc(-c2cccc(N(c3cccc4ccccc34)c3ccc(-c4cccc(-n5c6ccccc6c6ccccc65)c4)c4oc5ccccc5c34)c2)cc1 ; c1ccc(-c2ccccc2N(c2cccc3ccccc23)c2ccc(-c3cccc(-n4c5ccccc5c5ccccc54)c3)c3oc4ccccc4c23)cc1]

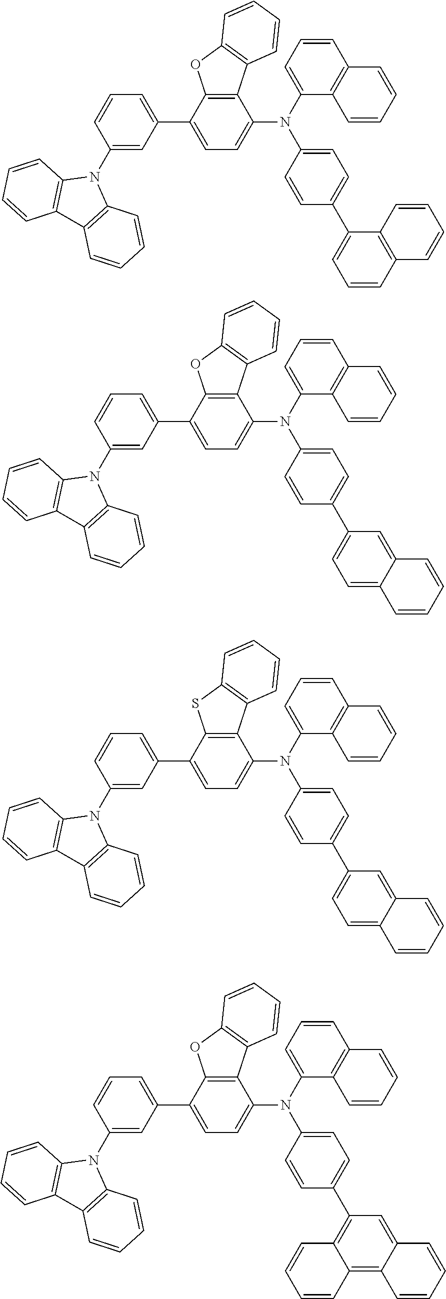 [SMILES: c1cc(-c2ccc(N(c3ccc(-c4cccc5ccccc45)cc3)c3cccc4ccccc34)c3c2oc2ccccc23)cc(-n2c3ccccc3c3ccccc32)c1 ; c1cc(-c2ccc(N(c3ccc(-c4ccc5ccccc5c4)cc3)c3cccc4ccccc34)c3c2oc2ccccc23)cc(-n2c3ccccc3c3ccccc32)c1 ; c1cc(-c2ccc(N(c3ccc(-c4ccc5ccccc5c4)cc3)c3cccc4ccccc34)c3c2sc2ccccc23)cc(-n2c3ccccc3c3ccccc32)c1 ; c1cc(-c2ccc(N(c3ccc(-c4cc5ccccc5c5ccccc45)cc3)c3cccc4ccccc34)c3c2oc2ccccc23)cc(-n2c3ccccc3c3ccccc32)c1]

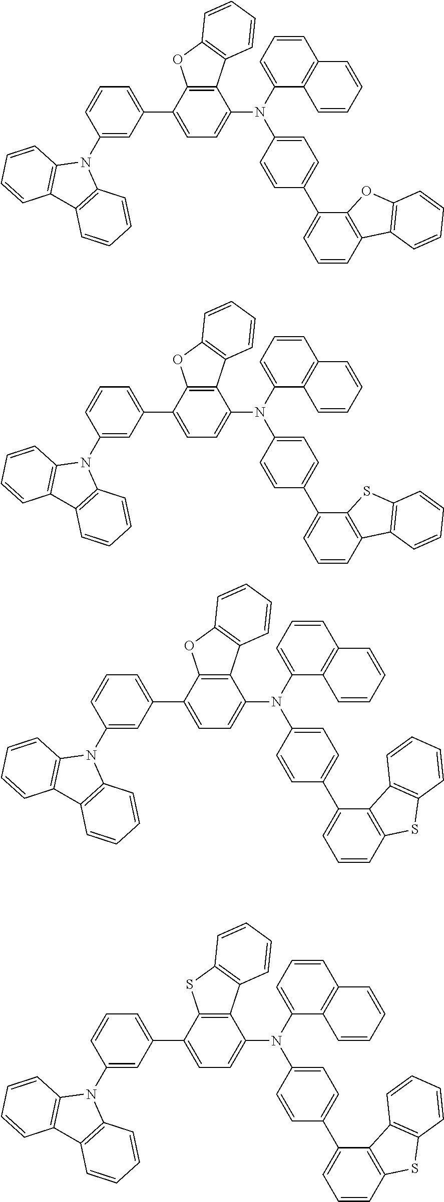 [SMILES: c1cc(-c2ccc(N(c3ccc(-c4cccc5c4oc4ccccc45)cc3)c3cccc4ccccc34)c3c2oc2ccccc23)cc(-n2c3ccccc3c3ccccc32)c1 ; c1cc(-c2ccc(N(c3ccc(-c4cccc5c4sc4ccccc45)cc3)c3cccc4ccccc34)c3c2oc2ccccc23)cc(-n2c3ccccc3c3ccccc32)c1 ; c1cc(-c2ccc(N(c3ccc(-c4cccc5sc6ccccc6c45)cc3)c3cccc4ccccc34)c3c2oc2ccccc23)cc(-n2c3ccccc3c3ccccc32)c1 ; c1cc(-c2ccc(N(c3ccc(-c4cccc5sc6ccccc6c45)cc3)c3cccc4ccccc34)c3c2sc2ccccc23)cc(-n2c3ccccc3c3ccccc32)c1]

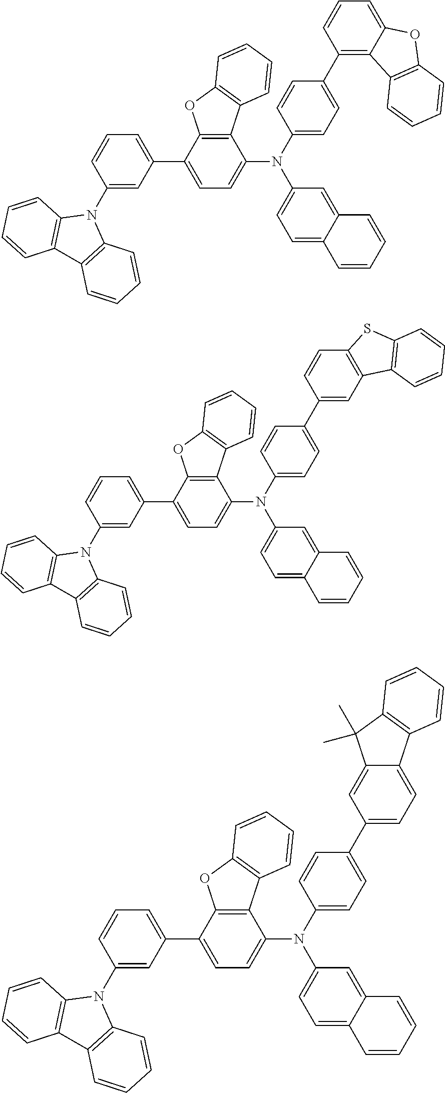 [SMILES: c1cc(-c2ccc(N(c3ccc(-c4cccc5oc6ccccc6c45)cc3)c3ccc4ccccc4c3)c3c2oc2ccccc23)cc(-n2c3ccccc3c3ccccc32)c1 ; c1cc(-c2ccc(N(c3ccc(-c4ccc5sc6ccccc6c5c4)cc3)c3ccc4ccccc4c3)c3c2oc2ccccc23)cc(-n2c3ccccc3c3ccccc32)c1 ; CC1(C)c2ccccc2-c2ccc(-c3ccc(N(c4ccc5ccccc5c4)c4ccc(-c5cccc(-n6c7ccccc7c7ccccc76)c5)c5oc6ccccc6c45)cc3)cc21]

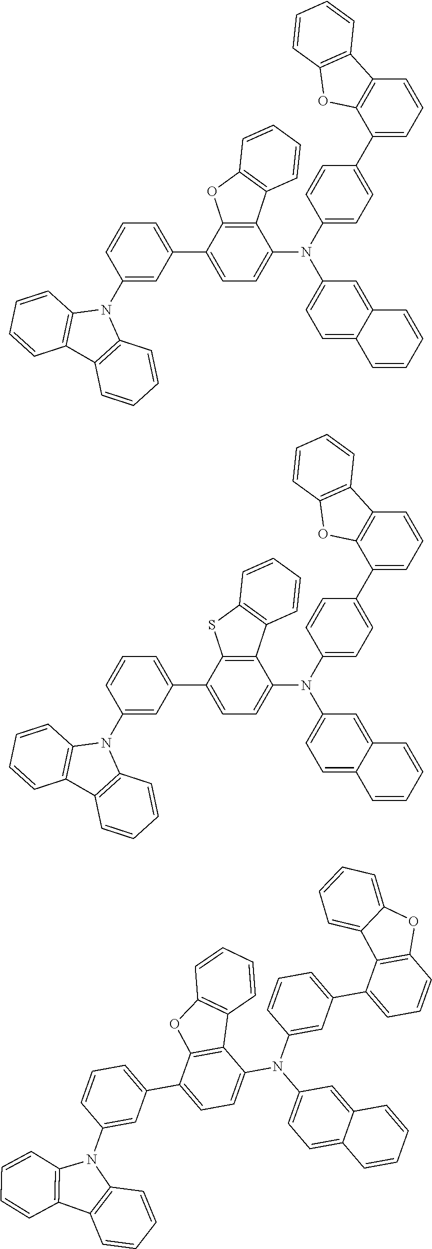 [SMILES: c1cc(-c2ccc(N(c3ccc(-c4cccc5c4oc4ccccc45)cc3)c3ccc4ccccc4c3)c3c2oc2ccccc23)cc(-n2c3ccccc3c3ccccc32)c1 ; c1cc(-c2ccc(N(c3ccc(-c4cccc5c4oc4ccccc45)cc3)c3ccc4ccccc4c3)c3c2sc2ccccc23)cc(-n2c3ccccc3c3ccccc32)c1 ; c1cc(-c2cccc3oc4ccccc4c23)cc(N(c2ccc3ccccc3c2)c2ccc(-c3cccc(-n4c5ccccc5c5ccccc54)c3)c3oc4ccccc4c23)c1]

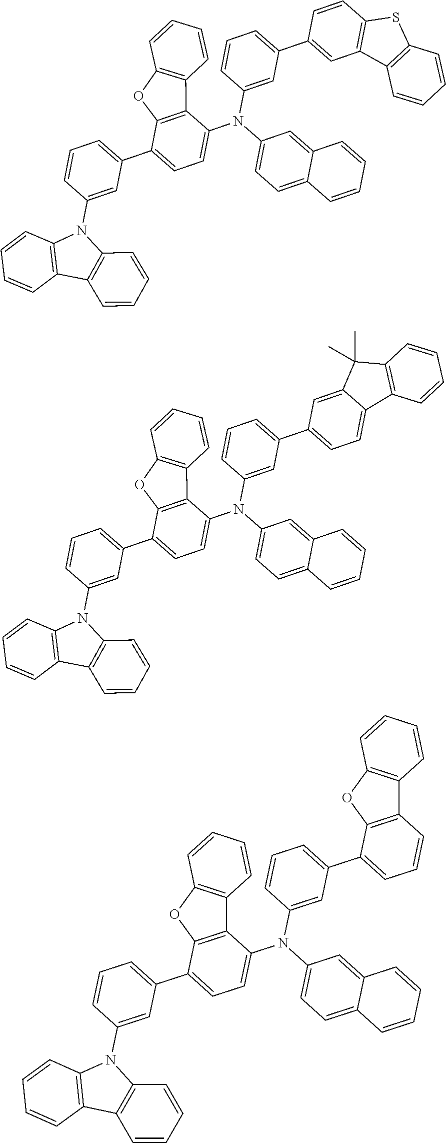 [SMILES: c1cc(-c2ccc3sc4ccccc4c3c2)cc(N(c2ccc3ccccc3c2)c2ccc(-c3cccc(-n4c5ccccc5c5ccccc54)c3)c3oc4ccccc4c23)c1 ; CC1(C)c2ccccc2-c2ccc(-c3cccc(N(c4ccc5ccccc5c4)c4ccc(-c5cccc(-n6c7ccccc7c7ccccc76)c5)c5oc6ccccc6c45)c3)cc21 ; c1cc(-c2cccc3c2oc2ccccc23)cc(N(c2ccc3ccccc3c2)c2ccc(-c3cccc(-n4c5ccccc5c5ccccc54)c3)c3oc4ccccc4c23)c1]

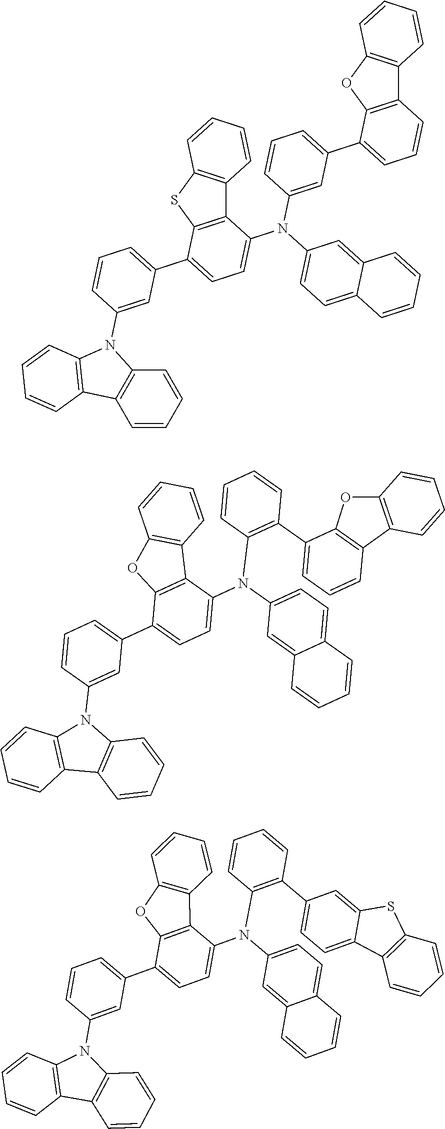

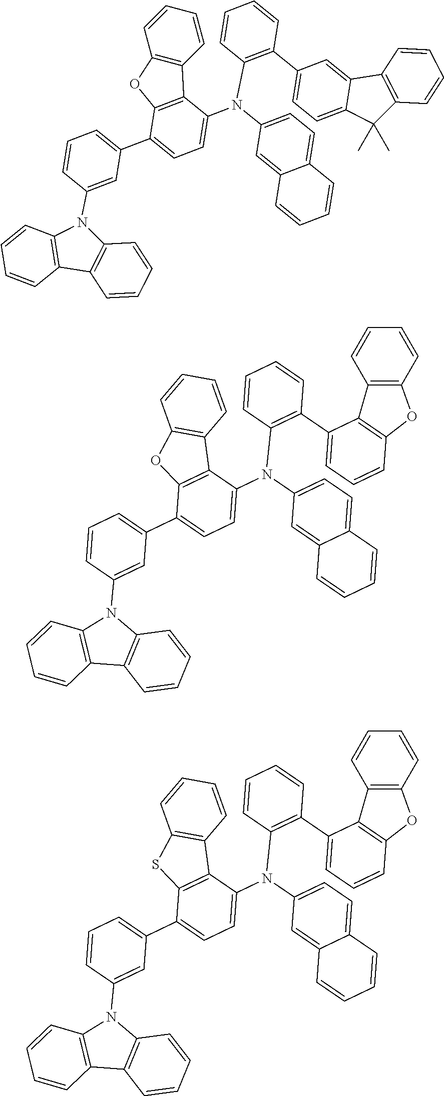 [SMILES: CC1(C)c2ccccc2-c2cc(-c3ccccc3N(c3ccc4ccccc4c3)c3ccc(-c4cccc(-n5c6ccccc6c6ccccc65)c4)c4oc5ccccc5c34)ccc21 ; c1cc(-c2ccc(N(c3ccc4ccccc4c3)c3ccccc3-c3cccc4oc5ccccc5c34)c3c2oc2ccccc23)cc(-n2c3ccccc3c3ccccc32)c1 ; c1cc(-c2ccc(N(c3ccc4ccccc4c3)c3ccccc3-c3cccc4oc5ccccc5c34)c3c2sc2ccccc23)cc(-n2c3ccccc3c3ccccc32)c1]

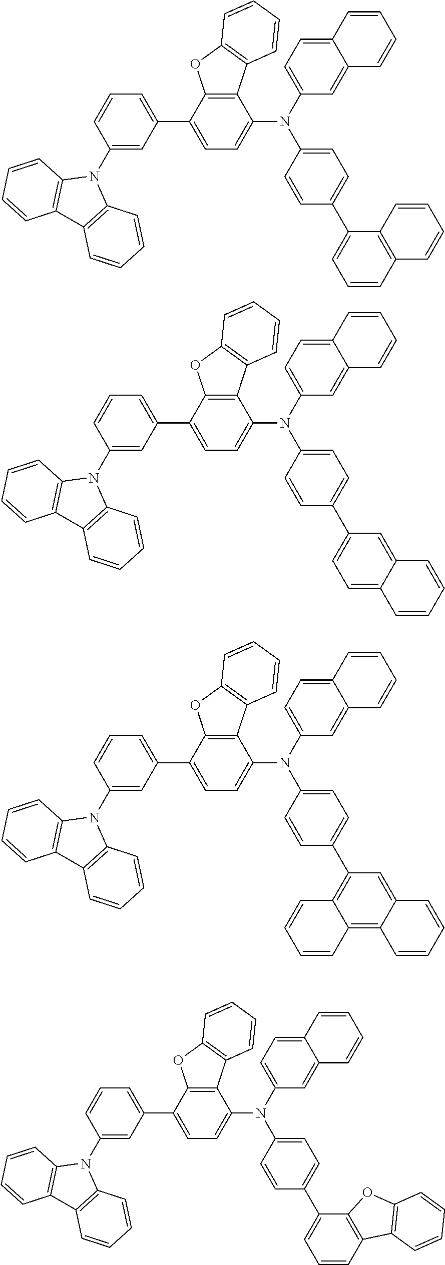

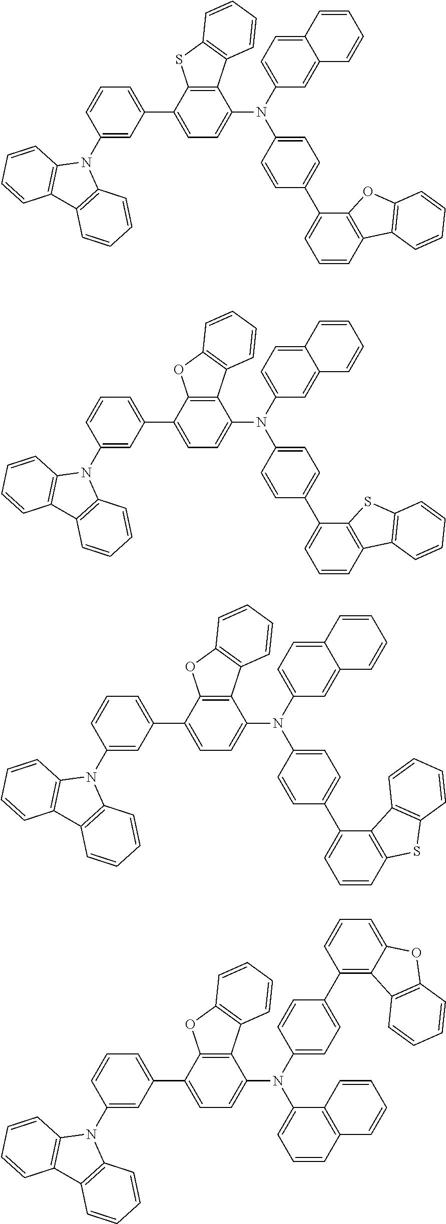

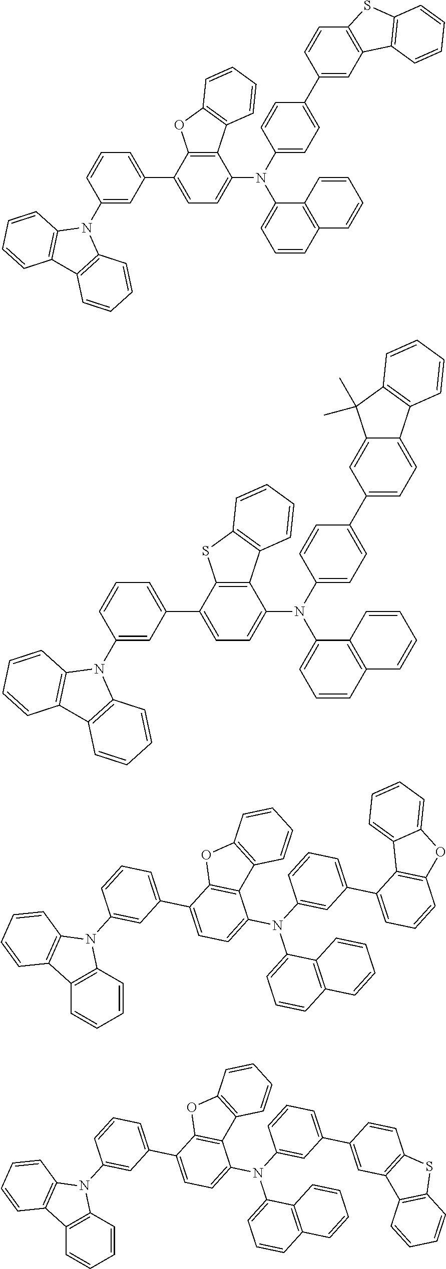 [SMILES: c1cc(-c2ccc(N(c3ccc(-c4ccc5sc6ccccc6c5c4)cc3)c3cccc4ccccc34)c3c2oc2ccccc23)cc(-n2c3ccccc3c3ccccc32)c1 ; CC1(C)c2ccccc2-c2ccc(-c3ccc(N(c4cccc5ccccc45)c4ccc(-c5cccc(-n6c7ccccc7c7ccccc76)c5)c5sc6ccccc6c45)cc3)cc21 ; c1cc(-c2cccc3oc4ccccc4c23)cc(N(c2cccc3ccccc23)c2ccc(-c3cccc(-n4c5ccccc5c5ccccc54)c3)c3oc4ccccc4c23)c1 ; c1cc(-c2ccc3sc4ccccc4c3c2)cc(N(c2cccc3ccccc23)c2ccc(-c3cccc(-n4c5ccccc5c5ccccc54)c3)c3oc4ccccc4c23)c1]

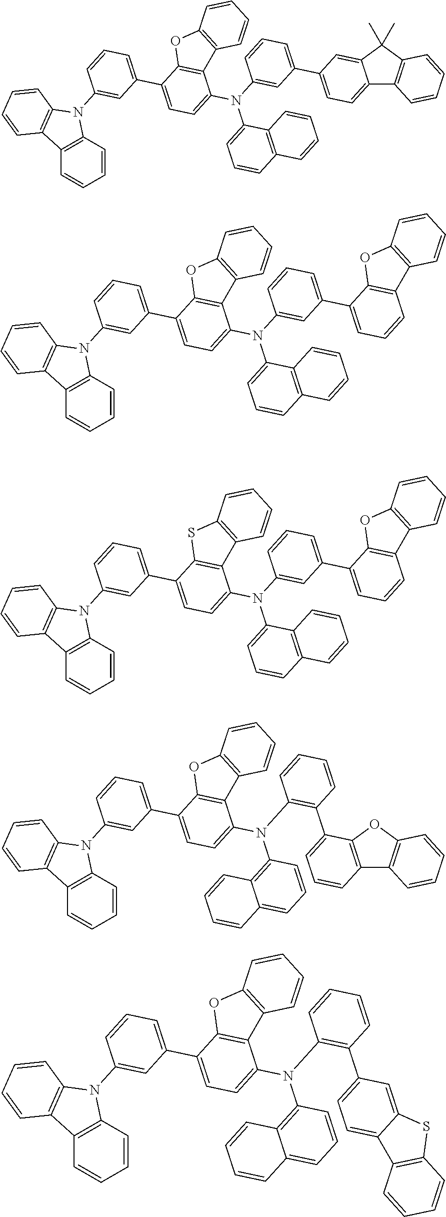

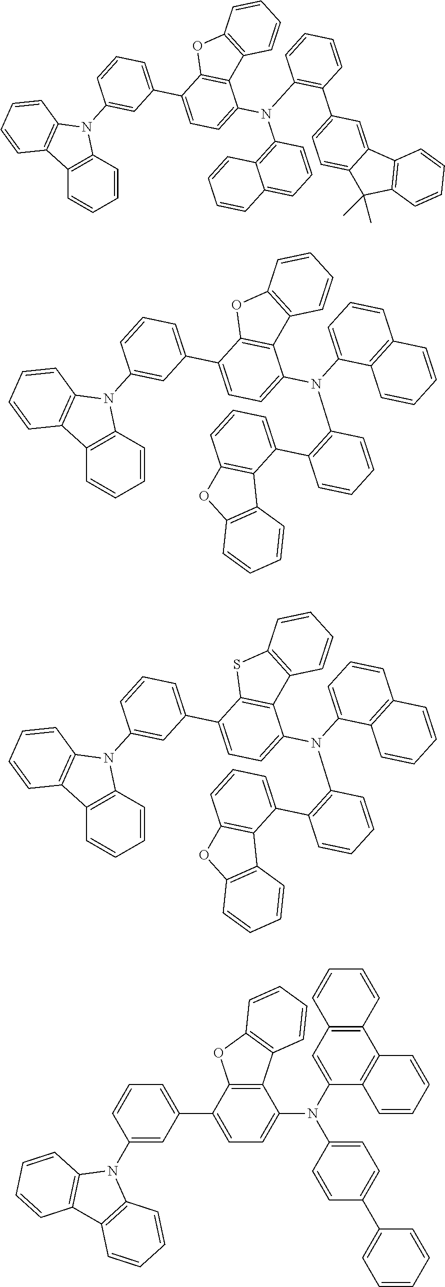 [SMILES: CC1(C)c2ccccc2-c2cc(-c3ccccc3N(c3cccc4ccccc34)c3ccc(-c4cccc(-n5c6ccccc6c6ccccc65)c4)c4oc5ccccc5c34)ccc21 ; c1cc(-c2ccc(N(c3ccccc3-c3cccc4oc5ccccc5c34)c3cccc4ccccc34)c3c2oc2ccccc23)cc(-n2c3ccccc3c3ccccc32)c1 ; c1cc(-c2ccc(N(c3ccccc3-c3cccc4oc5ccccc5c34)c3cccc4ccccc34)c3c2sc2ccccc23)cc(-n2c3ccccc3c3ccccc32)c1 ; c1ccc(-c2ccc(N(c3cc4ccccc4c4ccccc34)c3ccc(-c4cccc(-n5c6ccccc6c6ccccc65)c4)c4oc5ccccc5c34)cc2)cc1]

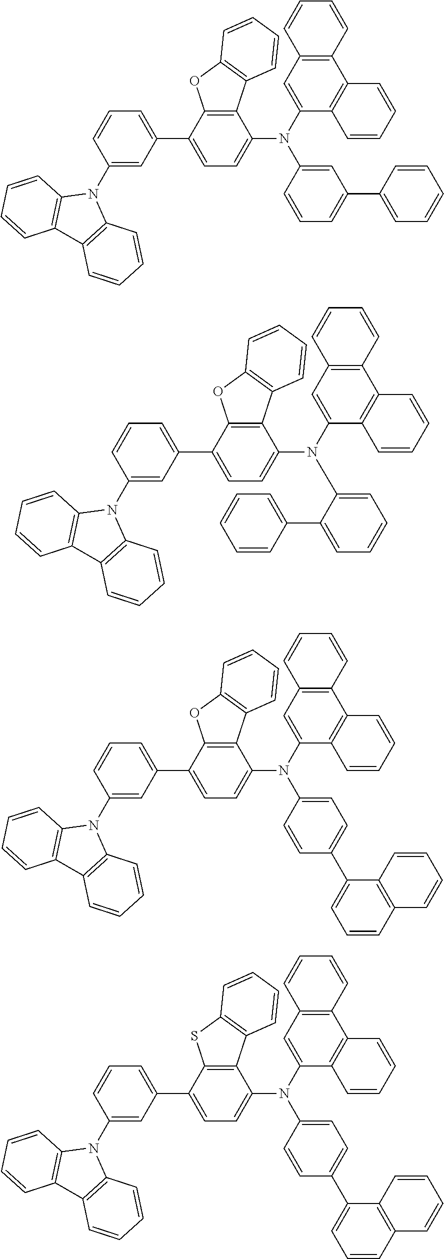

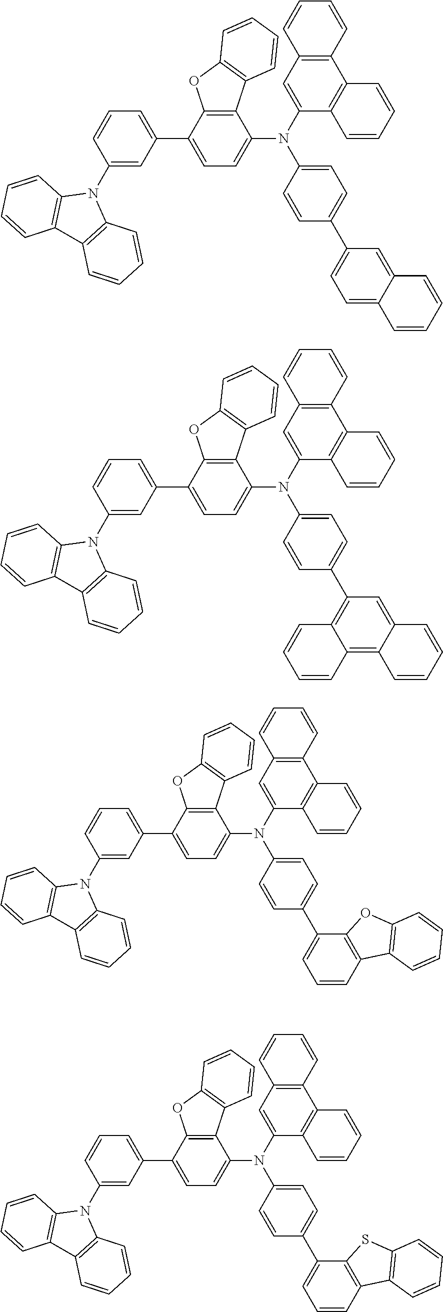

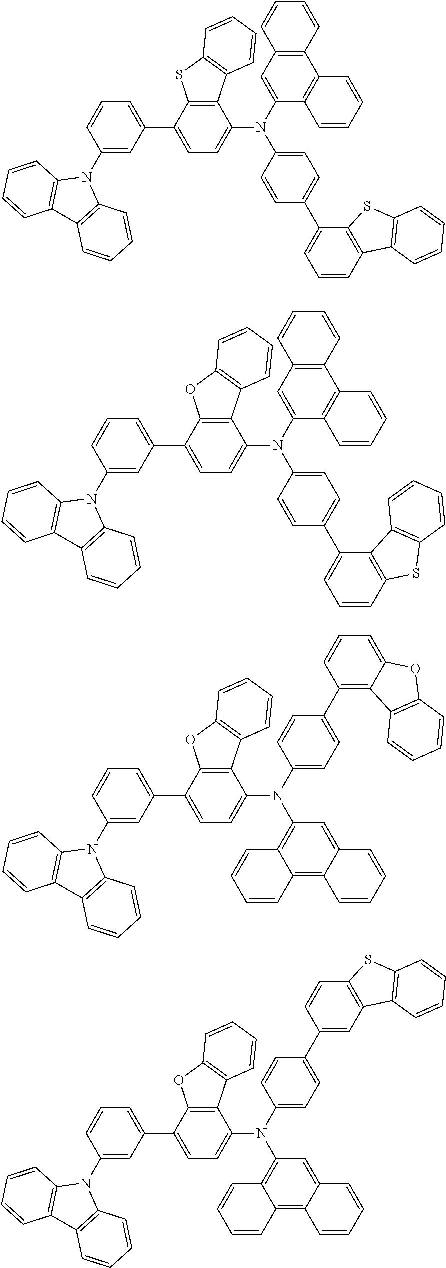

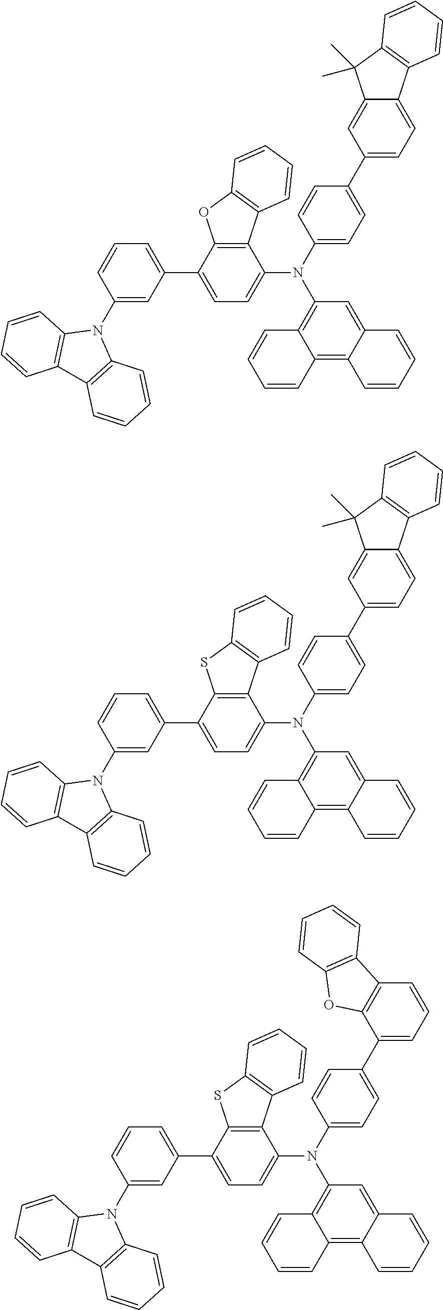 [SMILES: CC1(C)c2ccccc2-c2ccc(-c3ccc(N(c4cc5ccccc5c5ccccc45)c4ccc(-c5cccc(-n6c7ccccc7c7ccccc76)c5)c5oc6ccccc6c45)cc3)cc21 ; CC1(C)c2ccccc2-c2ccc(-c3ccc(N(c4cc5ccccc5c5ccccc45)c4ccc(-c5cccc(-n6c7ccccc7c7ccccc76)c5)c5sc6ccccc6c45)cc3)cc21 ; c1cc(-c2ccc(N(c3ccc(-c4cccc5c4oc4ccccc45)cc3)c3cc4ccccc4c4ccccc34)c3c2sc2ccccc23)cc(-n2c3ccccc3c3ccccc32)c1]

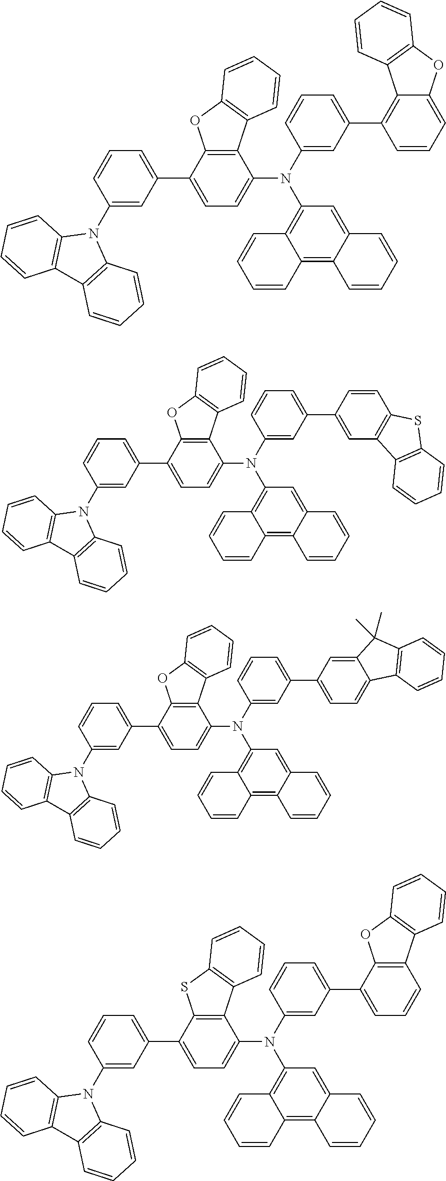 [SMILES: c1cc(-c2cccc3oc4ccccc4c23)cc(N(c2cc3ccccc3c3ccccc23)c2ccc(-c3cccc(-n4c5ccccc5c5ccccc54)c3)c3oc4ccccc4c23)c1 ; c1cc(-c2ccc3sc4ccccc4c3c2)cc(N(c2cc3ccccc3c3ccccc23)c2ccc(-c3cccc(-n4c5ccccc5c5ccccc54)c3)c3oc4ccccc4c23)c1 ; CC1(C)c2ccccc2-c2ccc(-c3cccc(N(c4cc5ccccc5c5ccccc45)c4ccc(-c5cccc(-n6c7ccccc7c7ccccc76)c5)c5oc6ccccc6c45)c3)cc21 ; c1cc(-c2cccc3c2oc2ccccc23)cc(N(c2cc3ccccc3c3ccccc23)c2ccc(-c3cccc(-n4c5ccccc5c5ccccc54)c3)c3sc4ccccc4c23)c1]

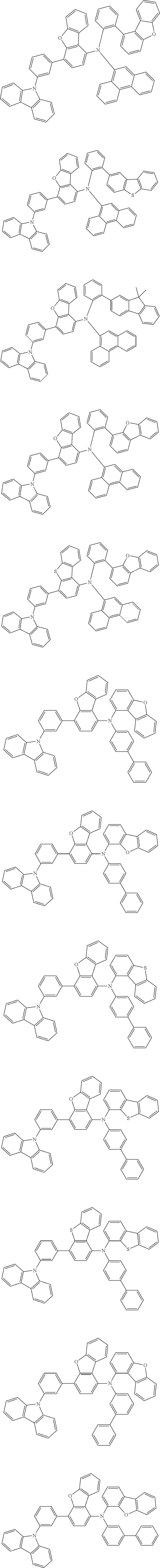

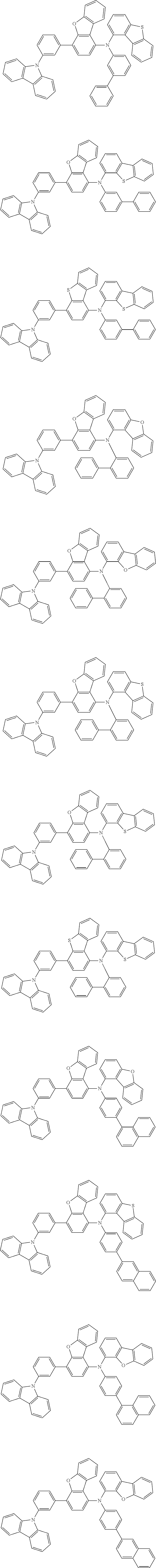

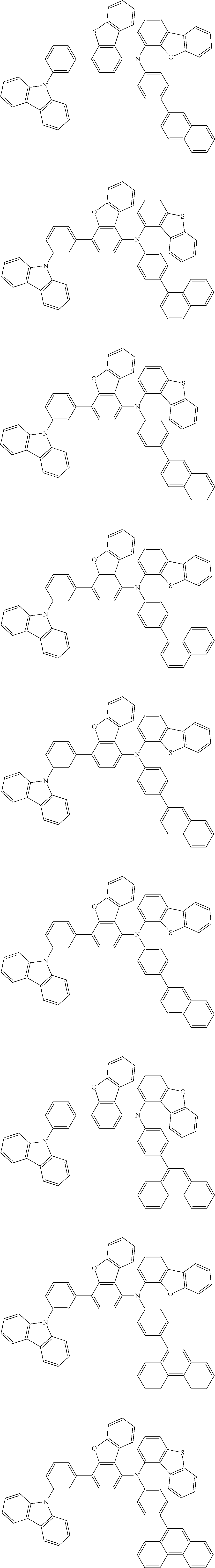

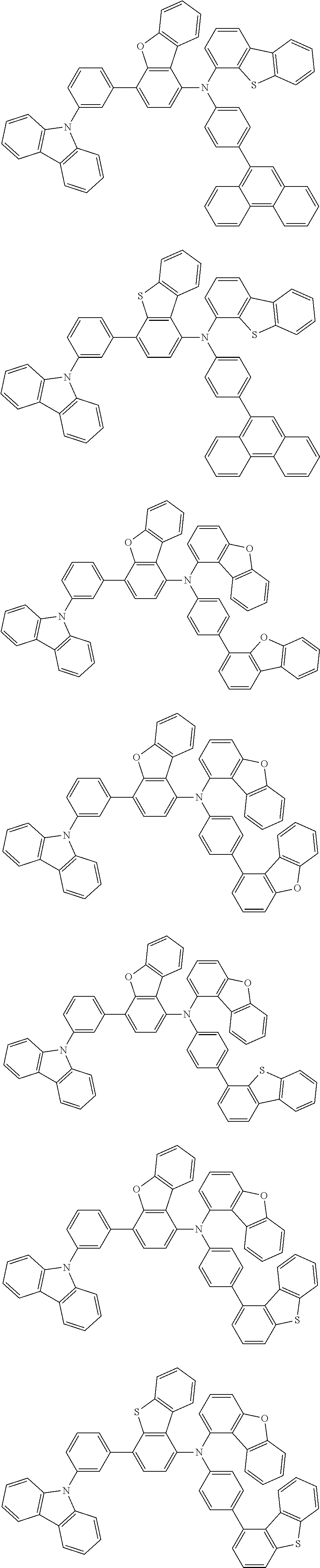

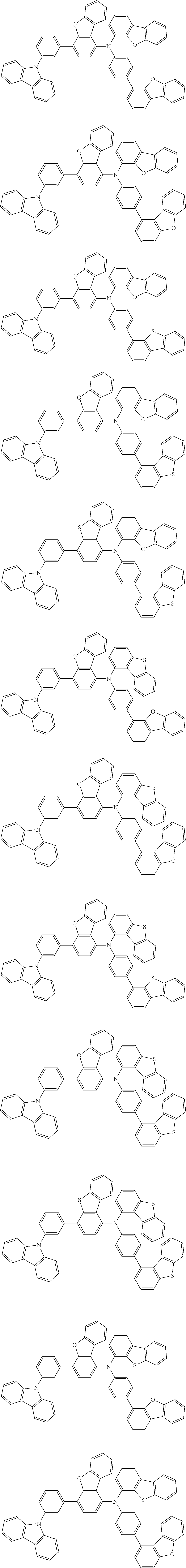

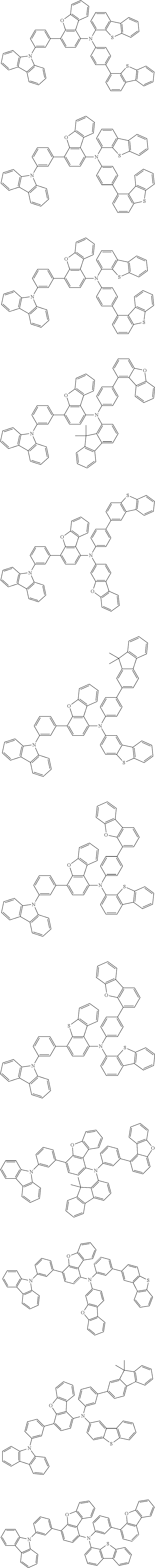

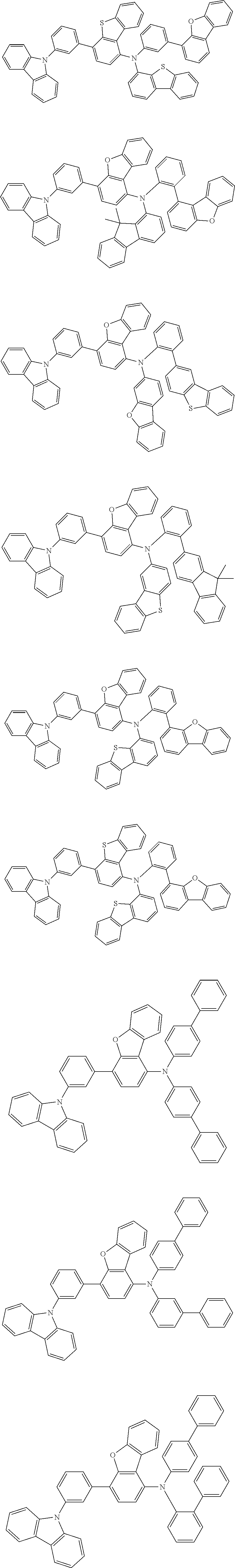

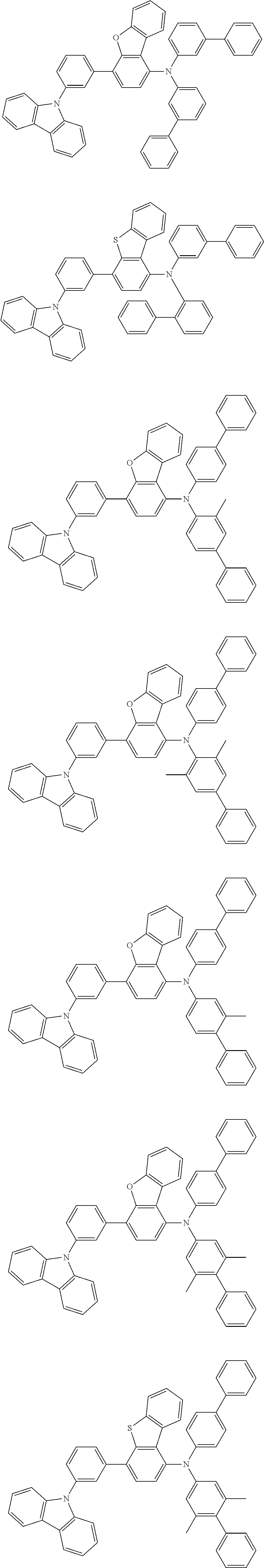

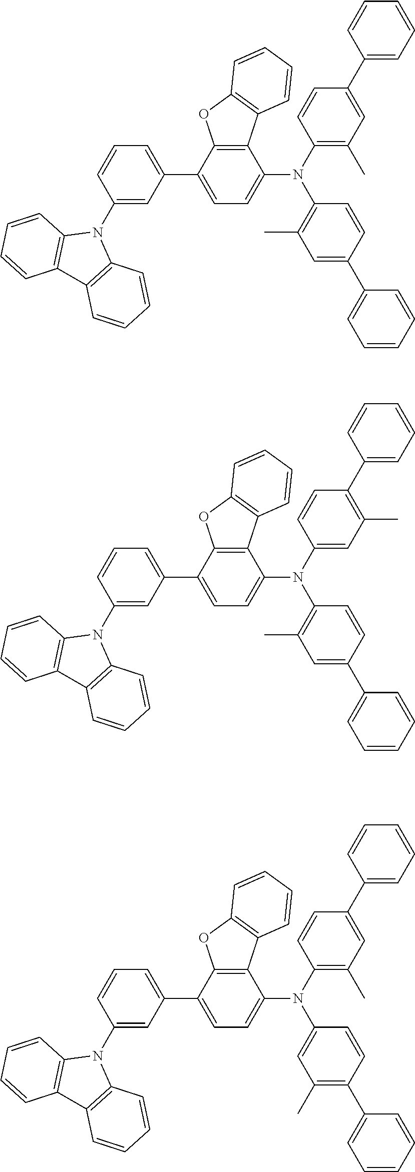 [SMILES: Cc1cc(-c2ccccc2)ccc1N(c1ccc(-c2ccccc2)cc1C)c1ccc(-c2cccc(-n3c4ccccc4c4ccccc43)c2)c2oc3ccccc3c12 ; Cc1cc(N(c2ccc(-c3ccccc3)cc2C)c2ccc(-c3cccc(-n4c5ccccc5c5ccccc54)c3)c3oc4ccccc4c23)ccc1-c1ccccc1 ; Cc1cc(N(c2ccc(-c3ccccc3)cc2C)c2ccc(-c3cccc(-n4c5ccccc5c5ccccc54)c3)c3oc4ccccc4c23)ccc1-c1ccccc1]

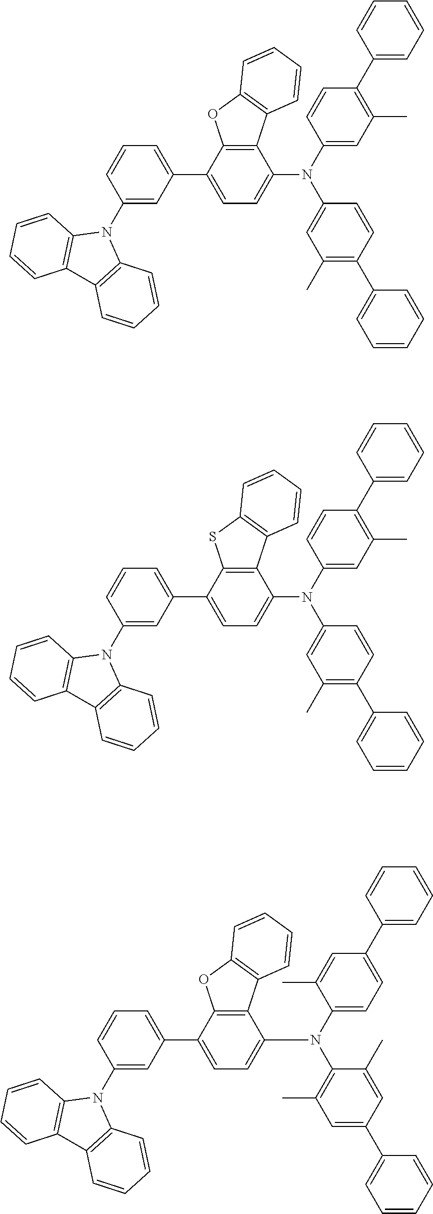 [SMILES: Cc1cc(N(c2ccc(-c3ccccc3)c(C)c2)c2ccc(-c3cccc(-n4c5ccccc5c5ccccc54)c3)c3oc4ccccc4c23)ccc1-c1ccccc1 ; Cc1cc(N(c2ccc(-c3ccccc3)c(C)c2)c2ccc(-c3cccc(-n4c5ccccc5c5ccccc54)c3)c3sc4ccccc4c23)ccc1-c1ccccc1 ; Cc1cc(-c2ccccc2)ccc1N(c1c(C)cc(-c2ccccc2)cc1C)c1ccc(-c2cccc(-n3c4ccccc4c4ccccc43)c2)c2oc3ccccc3c12]

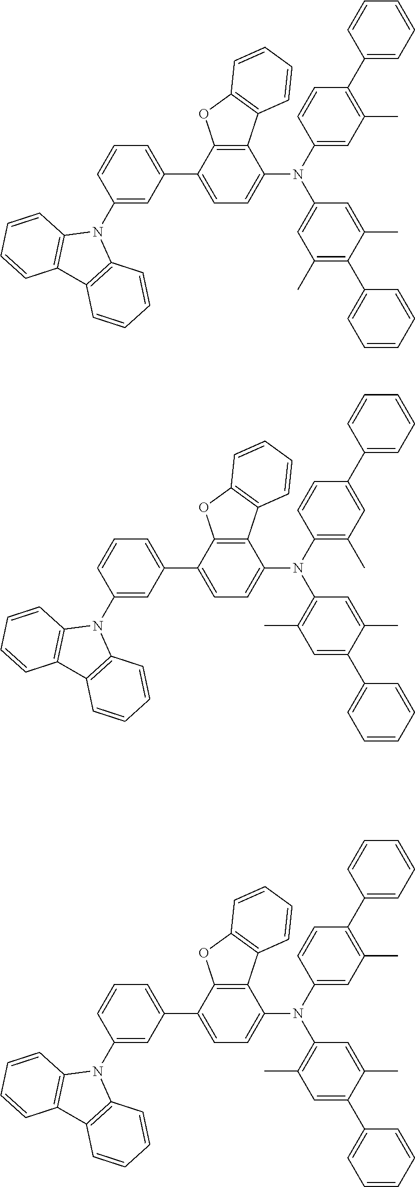 [SMILES: Cc1cc(N(c2cc(C)c(-c3ccccc3)c(C)c2)c2ccc(-c3cccc(-n4c5ccccc5c5ccccc54)c3)c3oc4ccccc4c23)ccc1-c1ccccc1 ; Cc1cc(N(c2ccc(-c3ccccc3)cc2C)c2ccc(-c3cccc(-n4c5ccccc5c5ccccc54)c3)c3oc4ccccc4c23)c(C)cc1-c1ccccc1 ; Cc1cc(N(c2cc(C)c(-c3ccccc3)cc2C)c2ccc(-c3cccc(-n4c5ccccc5c5ccccc54)c3)c3oc4ccccc4c23)ccc1-c1ccccc1]

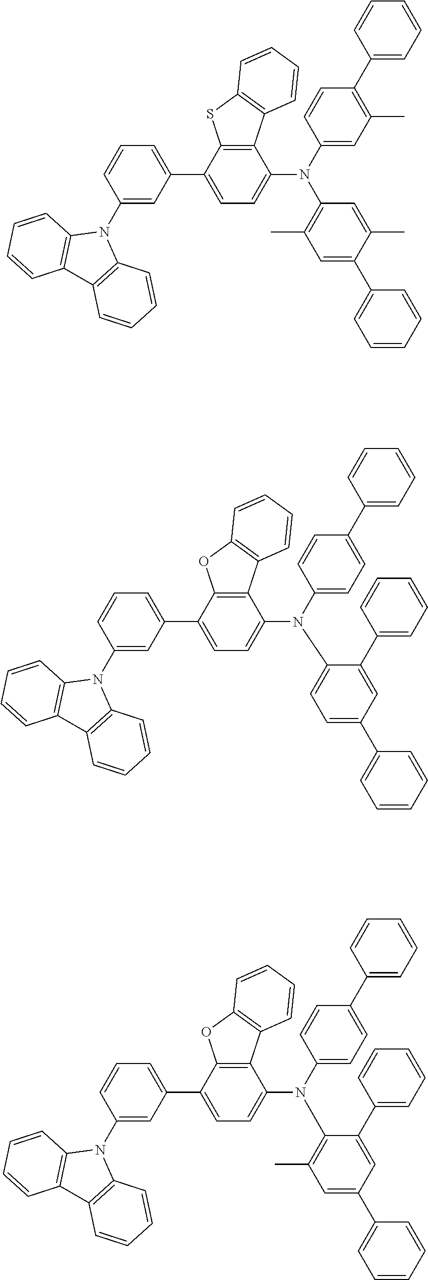 [SMILES: Cc1cc(N(c2cc(C)c(-c3ccccc3)cc2C)c2ccc(-c3cccc(-n4c5ccccc5c5ccccc54)c3)c3sc4ccccc4c23)ccc1-c1ccccc1 ; c1ccc(-c2ccc(N(c3ccc(-c4ccccc4)cc3-c3ccccc3)c3ccc(-c4cccc(-n5c6ccccc6c6ccccc65)c4)c4oc5ccccc5c34)cc2)cc1 ; Cc1cc(-c2ccccc2)cc(-c2ccccc2)c1N(c1ccc(-c2ccccc2)cc1)c1ccc(-c2cccc(-n3c4ccccc4c4ccccc43)c2)c2oc3ccccc3c12]

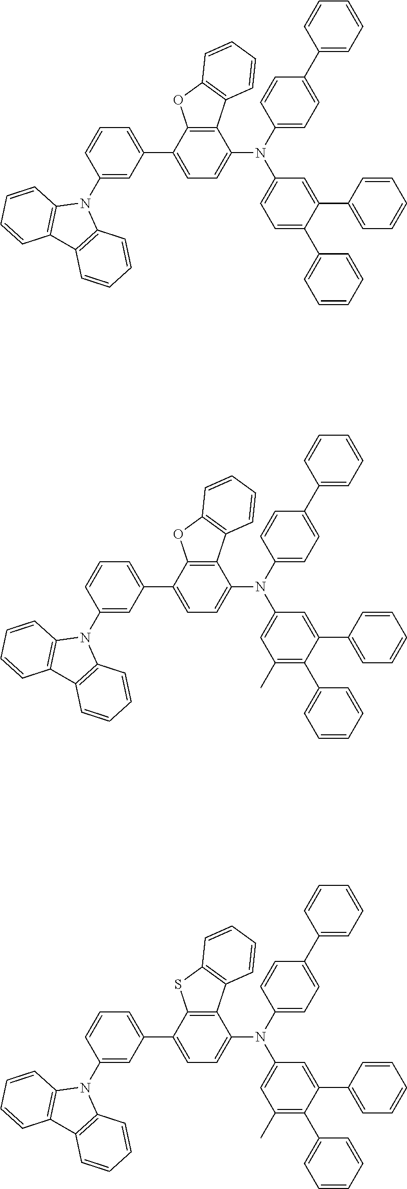 [SMILES: c1ccc(-c2ccc(N(c3ccc(-c4ccccc4)c(-c4ccccc4)c3)c3ccc(-c4cccc(-n5c6ccccc6c6ccccc65)c4)c4oc5ccccc5c34)cc2)cc1 ; Cc1cc(N(c2ccc(-c3ccccc3)cc2)c2ccc(-c3cccc(-n4c5ccccc5c5ccccc54)c3)c3oc4ccccc4c23)cc(-c2ccccc2)c1-c1ccccc1 ; Cc1cc(N(c2ccc(-c3ccccc3)cc2)c2ccc(-c3cccc(-n4c5ccccc5c5ccccc54)c3)c3sc4ccccc4c23)cc(-c2ccccc2)c1-c1ccccc1]

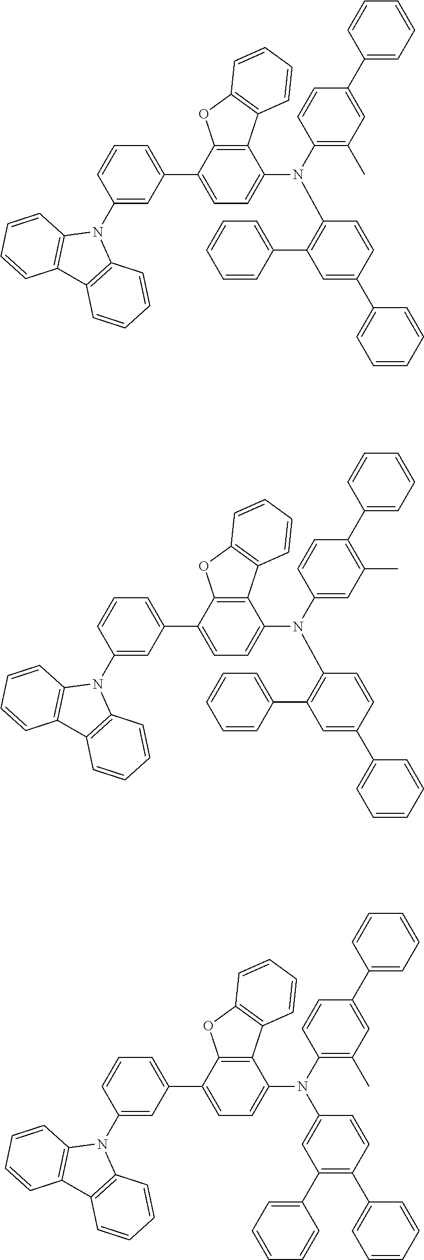 [SMILES: Cc1cc(-c2ccccc2)ccc1N(c1ccc(-c2ccccc2)cc1-c1ccccc1)c1ccc(-c2cccc(-n3c4ccccc4c4ccccc43)c2)c2oc3ccccc3c12 ; Cc1cc(N(c2ccc(-c3ccccc3)cc2-c2ccccc2)c2ccc(-c3cccc(-n4c5ccccc5c5ccccc54)c3)c3oc4ccccc4c23)ccc1-c1ccccc1 ; Cc1cc(-c2ccccc2)ccc1N(c1ccc(-c2ccccc2)c(-c2ccccc2)c1)c1ccc(-c2cccc(-n3c4ccccc4c4ccccc43)c2)c2oc3ccccc3c12]

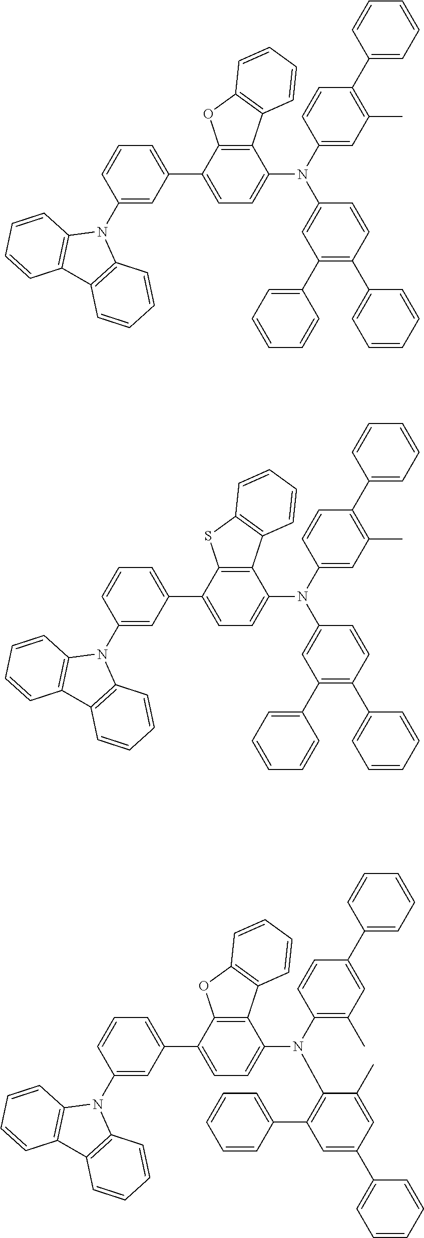 [SMILES: Cc1cc(N(c2ccc(-c3ccccc3)c(-c3ccccc3)c2)c2ccc(-c3cccc(-n4c5ccccc5c5ccccc54)c3)c3oc4ccccc4c23)ccc1-c1ccccc1 ; Cc1cc(N(c2ccc(-c3ccccc3)c(-c3ccccc3)c2)c2ccc(-c3cccc(-n4c5ccccc5c5ccccc54)c3)c3sc4ccccc4c23)ccc1-c1ccccc1 ; Cc1cc(-c2ccccc2)ccc1N(c1c(C)cc(-c2ccccc2)cc1-c1ccccc1)c1ccc(-c2cccc(-n3c4ccccc4c4ccccc43)c2)c2oc3ccccc3c12]

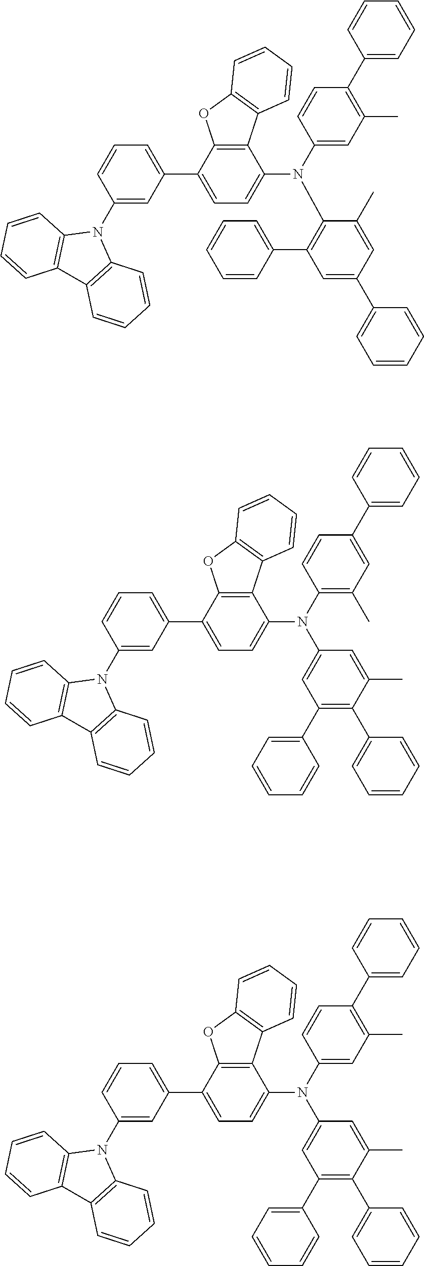 [SMILES: Cc1cc(N(c2c(C)cc(-c3ccccc3)cc2-c2ccccc2)c2ccc(-c3cccc(-n4c5ccccc5c5ccccc54)c3)c3oc4ccccc4c23)ccc1-c1ccccc1 ; Cc1cc(-c2ccccc2)ccc1N(c1cc(C)c(-c2ccccc2)c(-c2ccccc2)c1)c1ccc(-c2cccc(-n3c4ccccc4c4ccccc43)c2)c2oc3ccccc3c12 ; Cc1cc(N(c2cc(C)c(-c3ccccc3)c(-c3ccccc3)c2)c2ccc(-c3cccc(-n4c5ccccc5c5ccccc54)c3)c3oc4ccccc4c23)ccc1-c1ccccc1]

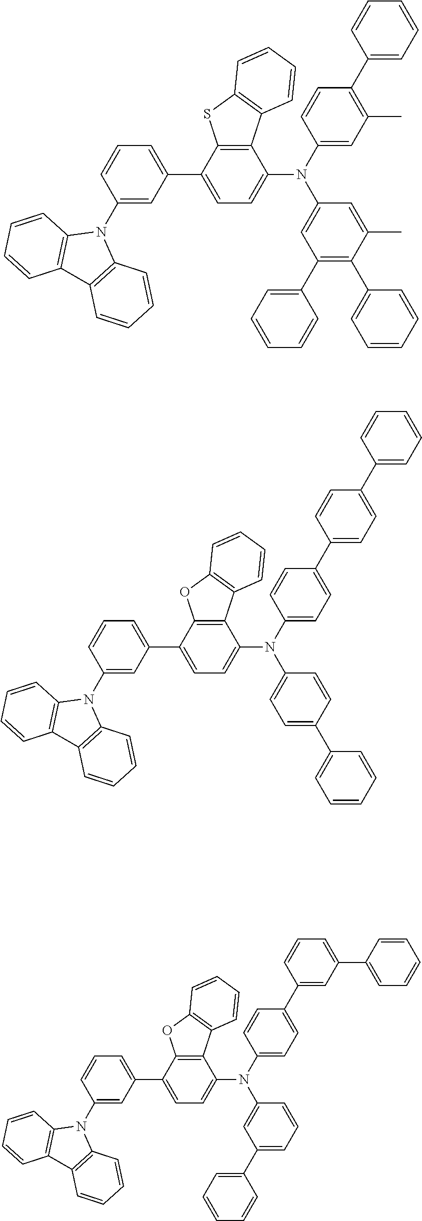 [SMILES: Cc1cc(N(c2cc(C)c(-c3ccccc3)c(-c3ccccc3)c2)c2ccc(-c3cccc(-n4c5ccccc5c5ccccc54)c3)c3sc4ccccc4c23)ccc1-c1ccccc1 ; c1ccc(-c2ccc(-c3ccc(N(c4ccc(-c5ccccc5)cc4)c4ccc(-c5cccc(-n6c7ccccc7c7ccccc76)c5)c5oc6ccccc6c45)cc3)cc2)cc1 ; c1ccc(-c2cccc(-c3ccc(N(c4cccc(-c5ccccc5)c4)c4ccc(-c5cccc(-n6c7ccccc7c7ccccc76)c5)c5oc6ccccc6c45)cc3)c2)cc1]

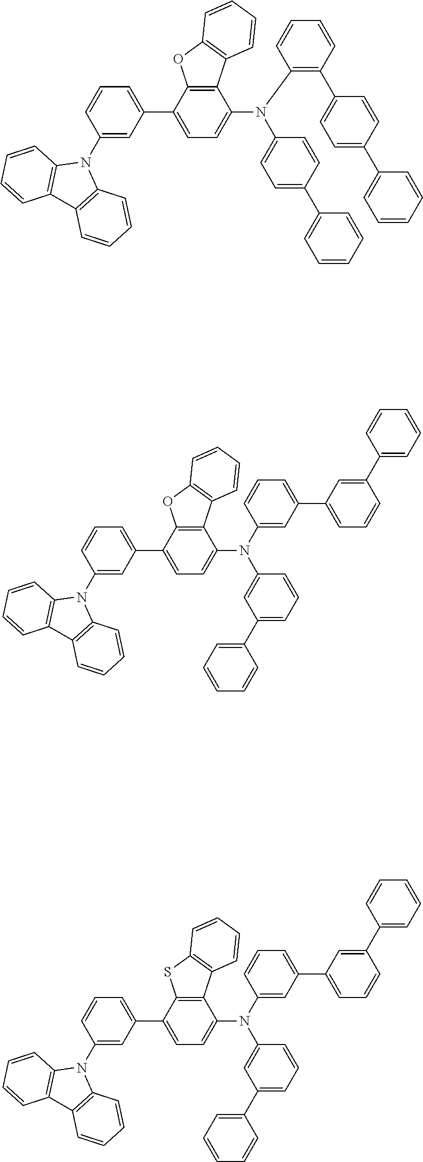 [SMILES: c1ccc(-c2ccc(-c3ccccc3N(c3ccc(-c4ccccc4)cc3)c3ccc(-c4cccc(-n5c6ccccc6c6ccccc65)c4)c4oc5ccccc5c34)cc2)cc1 ; c1ccc(-c2cccc(-c3cccc(N(c4cccc(-c5ccccc5)c4)c4ccc(-c5cccc(-n6c7ccccc7c7ccccc76)c5)c5oc6ccccc6c45)c3)c2)cc1 ; c1ccc(-c2cccc(-c3cccc(N(c4cccc(-c5ccccc5)c4)c4ccc(-c5cccc(-n6c7ccccc7c7ccccc76)c5)c5sc6ccccc6c45)c3)c2)cc1]

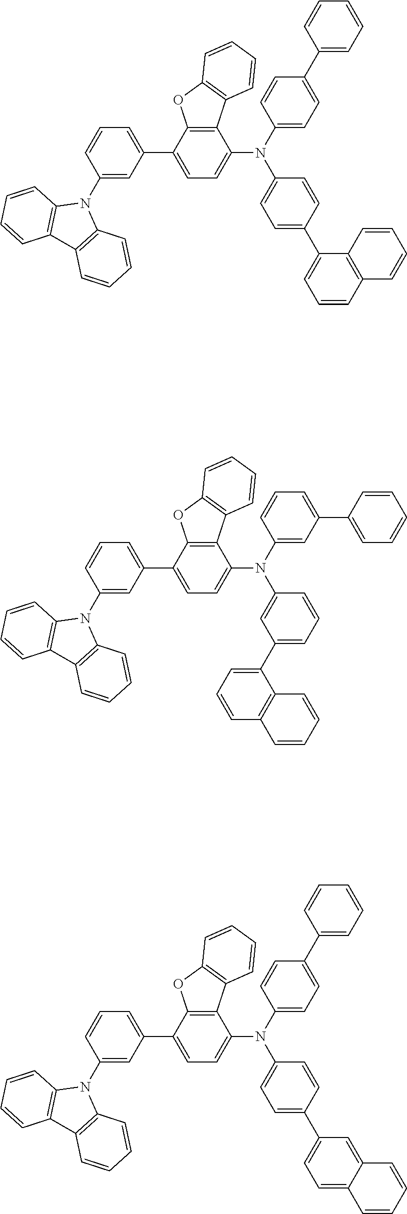 [SMILES: c1ccc(-c2ccc(N(c3ccc(-c4cccc5ccccc45)cc3)c3ccc(-c4cccc(-n5c6ccccc6c6ccccc65)c4)c4oc5ccccc5c34)cc2)cc1 ; c1ccc(-c2cccc(N(c3cccc(-c4cccc5ccccc45)c3)c3ccc(-c4cccc(-n5c6ccccc6c6ccccc65)c4)c4oc5ccccc5c34)c2)cc1 ; c1ccc(-c2ccc(N(c3ccc(-c4ccc5ccccc5c4)cc3)c3ccc(-c4cccc(-n5c6ccccc6c6ccccc65)c4)c4oc5ccccc5c34)cc2)cc1]

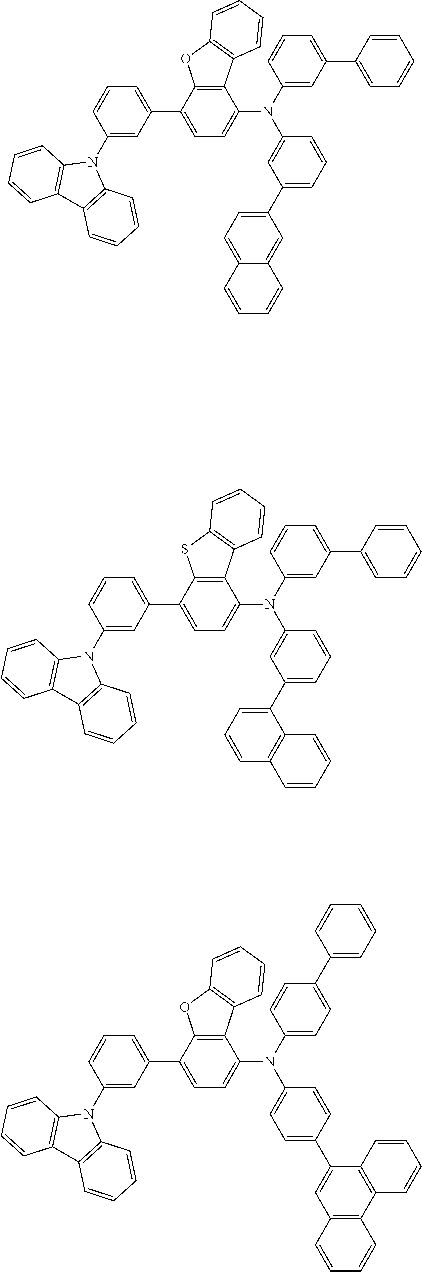 [SMILES: c1ccc(-c2cccc(N(c3cccc(-c4ccc5ccccc5c4)c3)c3ccc(-c4cccc(-n5c6ccccc6c6ccccc65)c4)c4oc5ccccc5c34)c2)cc1 ; c1ccc(-c2cccc(N(c3cccc(-c4cccc5ccccc45)c3)c3ccc(-c4cccc(-n5c6ccccc6c6ccccc65)c4)c4sc5ccccc5c34)c2)cc1 ; c1ccc(-c2ccc(N(c3ccc(-c4cc5ccccc5c5ccccc45)cc3)c3ccc(-c4cccc(-n5c6ccccc6c6ccccc65)c4)c4oc5ccccc5c34)cc2)cc1]

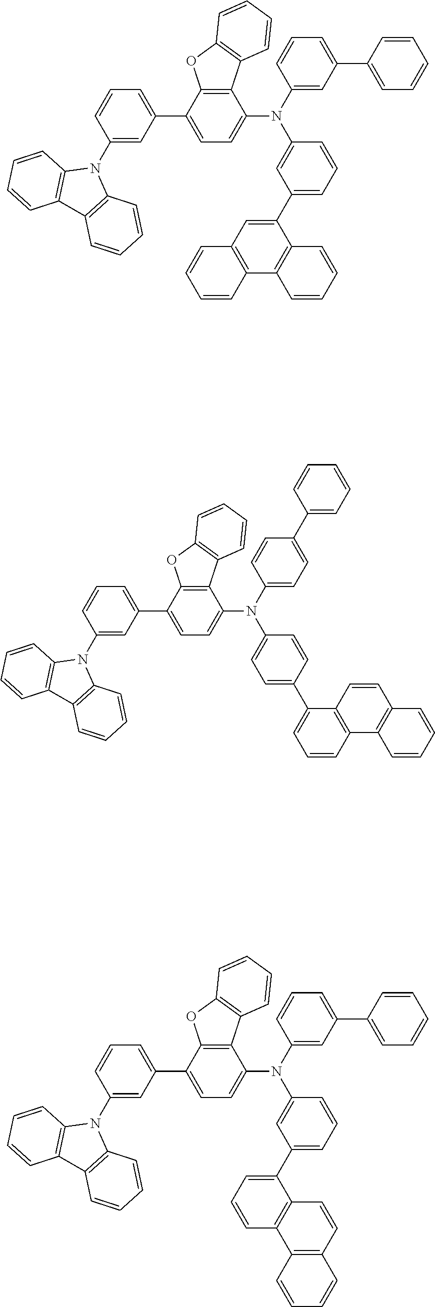 [SMILES: c1ccc(-c2cccc(N(c3cccc(-c4cc5ccccc5c5ccccc45)c3)c3ccc(-c4cccc(-n5c6ccccc6c6ccccc65)c4)c4oc5ccccc5c34)c2)cc1 ; c1ccc(-c2ccc(N(c3ccc(-c4cccc5c4ccc4ccccc45)cc3)c3ccc(-c4cccc(-n5c6ccccc6c6ccccc65)c4)c4oc5ccccc5c34)cc2)cc1 ; c1ccc(-c2cccc(N(c3cccc(-c4cccc5c4ccc4ccccc45)c3)c3ccc(-c4cccc(-n5c6ccccc6c6ccccc65)c4)c4oc5ccccc5c34)c2)cc1]

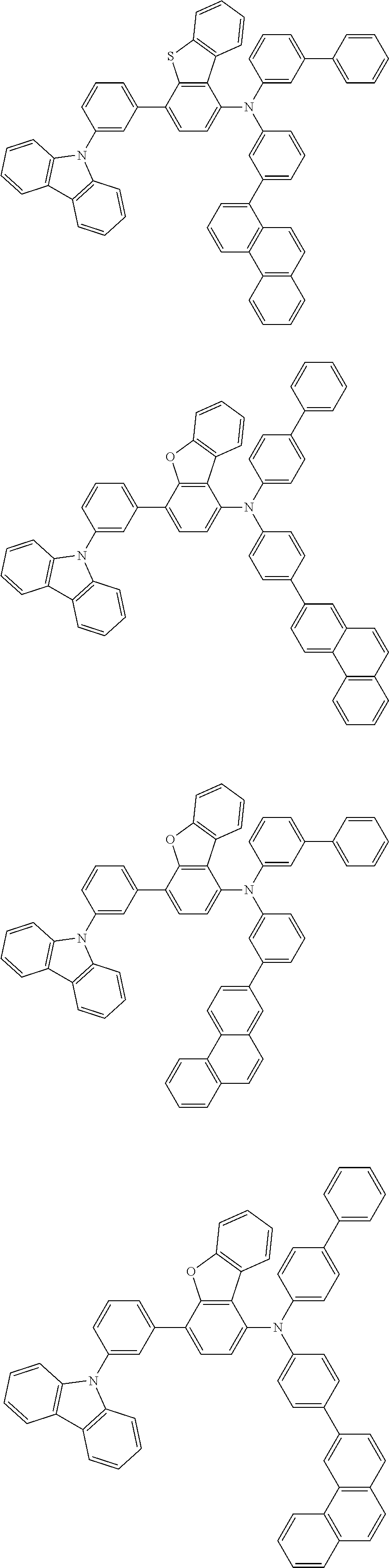 [SMILES: c1ccc(-c2cccc(N(c3cccc(-c4cccc5c4ccc4ccccc45)c3)c3ccc(-c4cccc(-n5c6ccccc6c6ccccc65)c4)c4sc5ccccc5c34)c2)cc1 ; c1ccc(-c2ccc(N(c3ccc(-c4ccc5c(ccc6ccccc65)c4)cc3)c3ccc(-c4cccc(-n5c6ccccc6c6ccccc65)c4)c4oc5ccccc5c34)cc2)cc1 ; c1ccc(-c2cccc(N(c3cccc(-c4ccc5c(ccc6ccccc65)c4)c3)c3ccc(-c4cccc(-n5c6ccccc6c6ccccc65)c4)c4oc5ccccc5c34)c2)cc1 ; c1ccc(-c2ccc(N(c3ccc(-c4ccc5ccc6ccccc6c5c4)cc3)c3ccc(-c4cccc(-n5c6ccccc6c6ccccc65)c4)c4oc5ccccc5c34)cc2)cc1]

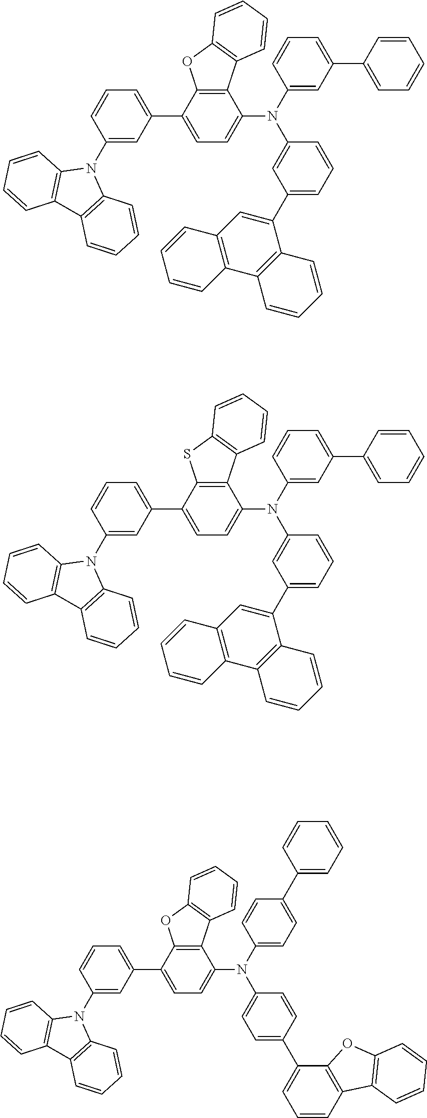 [SMILES: c1ccc(-c2cccc(N(c3cccc(-c4cc5ccccc5c5ccccc45)c3)c3ccc(-c4cccc(-n5c6ccccc6c6ccccc65)c4)c4oc5ccccc5c34)c2)cc1 ; c1ccc(-c2cccc(N(c3cccc(-c4cc5ccccc5c5ccccc45)c3)c3ccc(-c4cccc(-n5c6ccccc6c6ccccc65)c4)c4sc5ccccc5c34)c2)cc1 ; c1ccc(-c2ccc(N(c3ccc(-c4cccc5c4oc4ccccc45)cc3)c3ccc(-c4cccc(-n5c6ccccc6c6ccccc65)c4)c4oc5ccccc5c34)cc2)cc1]

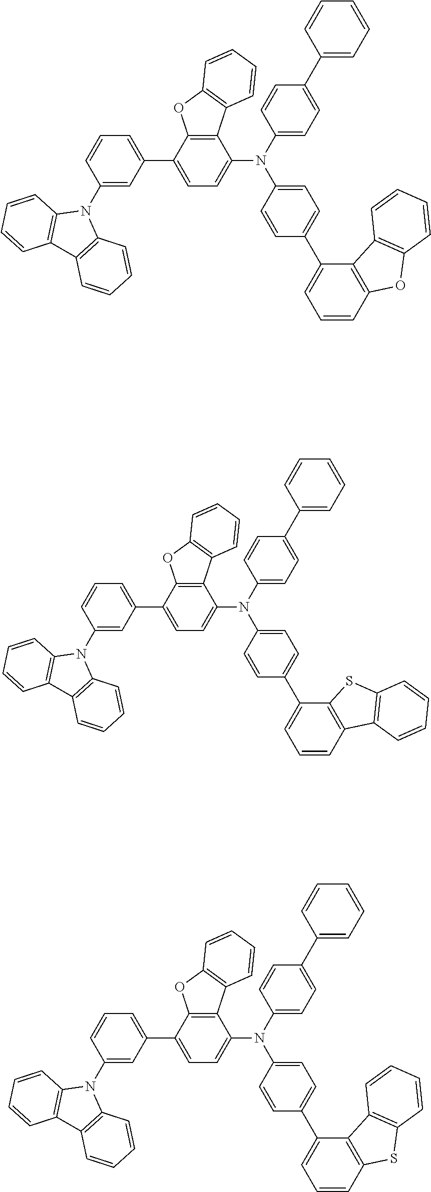 [SMILES: c1ccc(-c2ccc(N(c3ccc(-c4cccc5oc6ccccc6c45)cc3)c3ccc(-c4cccc(-n5c6ccccc6c6ccccc65)c4)c4oc5ccccc5c34)cc2)cc1 ; c1ccc(-c2ccc(N(c3ccc(-c4cccc5c4sc4ccccc45)cc3)c3ccc(-c4cccc(-n5c6ccccc6c6ccccc65)c4)c4oc5ccccc5c34)cc2)cc1 ; c1ccc(-c2ccc(N(c3ccc(-c4cccc5sc6ccccc6c45)cc3)c3ccc(-c4cccc(-n5c6ccccc6c6ccccc65)c4)c4oc5ccccc5c34)cc2)cc1]

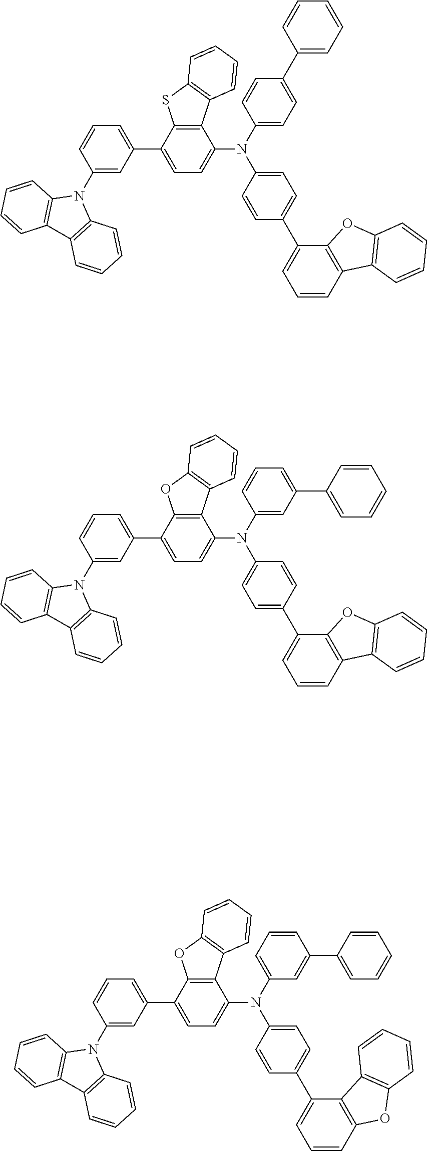 [SMILES: c1ccc(-c2ccc(N(c3ccc(-c4cccc5c4oc4ccccc45)cc3)c3ccc(-c4cccc(-n5c6ccccc6c6ccccc65)c4)c4sc5ccccc5c34)cc2)cc1 ; c1ccc(-c2cccc(N(c3ccc(-c4cccc5c4oc4ccccc45)cc3)c3ccc(-c4cccc(-n5c6ccccc6c6ccccc65)c4)c4oc5ccccc5c34)c2)cc1 ; c1ccc(-c2cccc(N(c3ccc(-c4cccc5oc6ccccc6c45)cc3)c3ccc(-c4cccc(-n5c6ccccc6c6ccccc65)c4)c4oc5ccccc5c34)c2)cc1]

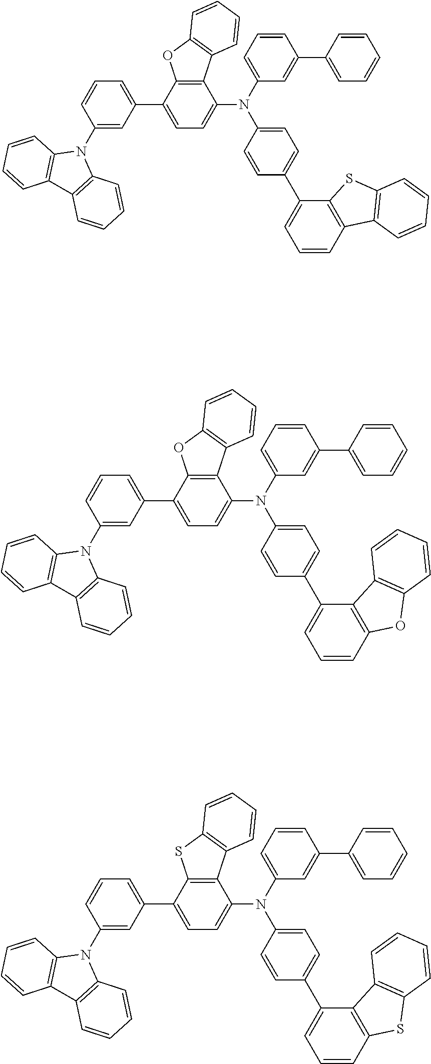 [SMILES: c1ccc(-c2cccc(N(c3ccc(-c4cccc5c4sc4ccccc45)cc3)c3ccc(-c4cccc(-n5c6ccccc6c6ccccc65)c4)c4oc5ccccc5c34)c2)cc1 ; c1ccc(-c2cccc(N(c3ccc(-c4cccc5oc6ccccc6c45)cc3)c3ccc(-c4cccc(-n5c6ccccc6c6ccccc65)c4)c4oc5ccccc5c34)c2)cc1 ; c1ccc(-c2cccc(N(c3ccc(-c4cccc5sc6ccccc6c45)cc3)c3ccc(-c4cccc(-n5c6ccccc6c6ccccc65)c4)c4sc5ccccc5c34)c2)cc1]

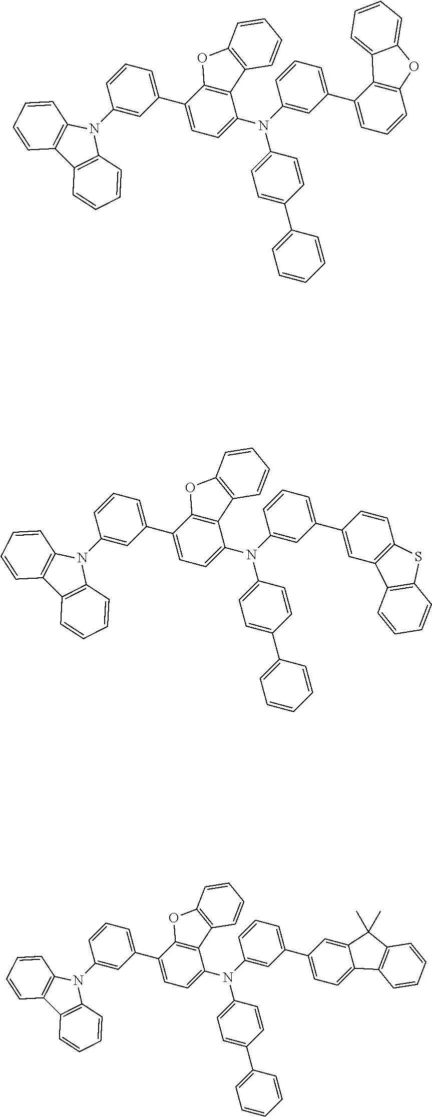 [SMILES: c1ccc(-c2ccc(N(c3cccc(-c4cccc5oc6ccccc6c45)c3)c3ccc(-c4cccc(-n5c6ccccc6c6ccccc65)c4)c4oc5ccccc5c34)cc2)cc1 ; c1ccc(-c2ccc(N(c3cccc(-c4ccc5sc6ccccc6c5c4)c3)c3ccc(-c4cccc(-n5c6ccccc6c6ccccc65)c4)c4oc5ccccc5c34)cc2)cc1 ; CC1(C)c2ccccc2-c2ccc(-c3cccc(N(c4ccc(-c5ccccc5)cc4)c4ccc(-c5cccc(-n6c7ccccc7c7ccccc76)c5)c5oc6ccccc6c45)c3)cc21]

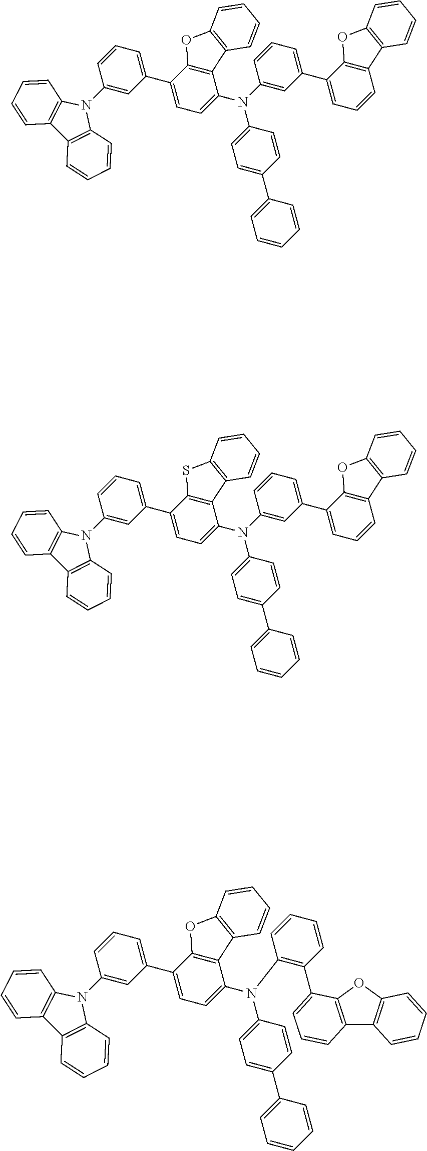 [SMILES: c1ccc(-c2ccc(N(c3cccc(-c4cccc5c4oc4ccccc45)c3)c3ccc(-c4cccc(-n5c6ccccc6c6ccccc65)c4)c4oc5ccccc5c34)cc2)cc1 ; c1ccc(-c2ccc(N(c3cccc(-c4cccc5c4oc4ccccc45)c3)c3ccc(-c4cccc(-n5c6ccccc6c6ccccc65)c4)c4sc5ccccc5c34)cc2)cc1 ; c1ccc(-c2ccc(N(c3ccccc3-c3cccc4c3oc3ccccc34)c3ccc(-c4cccc(-n5c6ccccc6c6ccccc65)c4)c4oc5ccccc5c34)cc2)cc1]

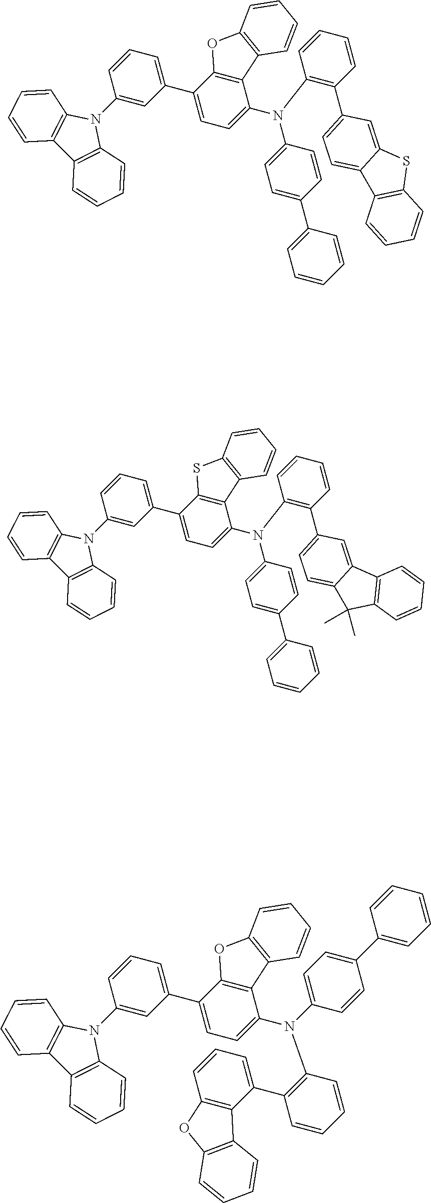 [SMILES: c1ccc(-c2ccc(N(c3ccccc3-c3ccc4c(c3)sc3ccccc34)c3ccc(-c4cccc(-n5c6ccccc6c6ccccc65)c4)c4oc5ccccc5c34)cc2)cc1 ; CC1(C)c2ccccc2-c2cc(-c3ccccc3N(c3ccc(-c4ccccc4)cc3)c3ccc(-c4cccc(-n5c6ccccc6c6ccccc65)c4)c4sc5ccccc5c34)ccc21 ; c1ccc(-c2ccc(N(c3ccccc3-c3cccc4oc5ccccc5c34)c3ccc(-c4cccc(-n5c6ccccc6c6ccccc65)c4)c4oc5ccccc5c34)cc2)cc1]

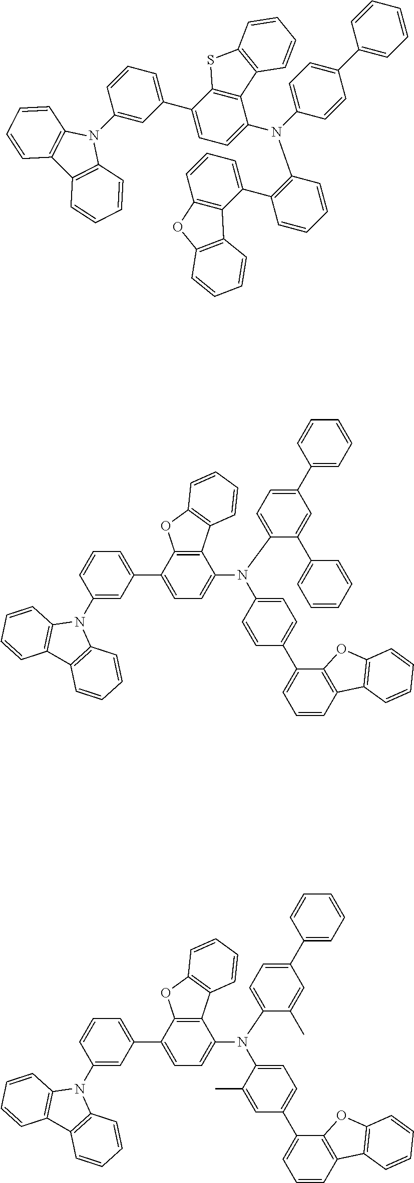 [SMILES: c1ccc(-c2ccc(N(c3ccccc3-c3cccc4oc5ccccc5c34)c3ccc(-c4cccc(-n5c6ccccc6c6ccccc65)c4)c4sc5ccccc5c34)cc2)cc1 ; c1ccc(-c2ccc(N(c3ccc(-c4cccc5c4oc4ccccc45)cc3)c3ccc(-c4cccc(-n5c6ccccc6c6ccccc65)c4)c4oc5ccccc5c34)c(-c3ccccc3)c2)cc1 ; Cc1cc(-c2ccccc2)ccc1N(c1ccc(-c2cccc3c2oc2ccccc23)cc1C)c1ccc(-c2cccc(-n3c4ccccc4c4ccccc43)c2)c2oc3ccccc3c12]

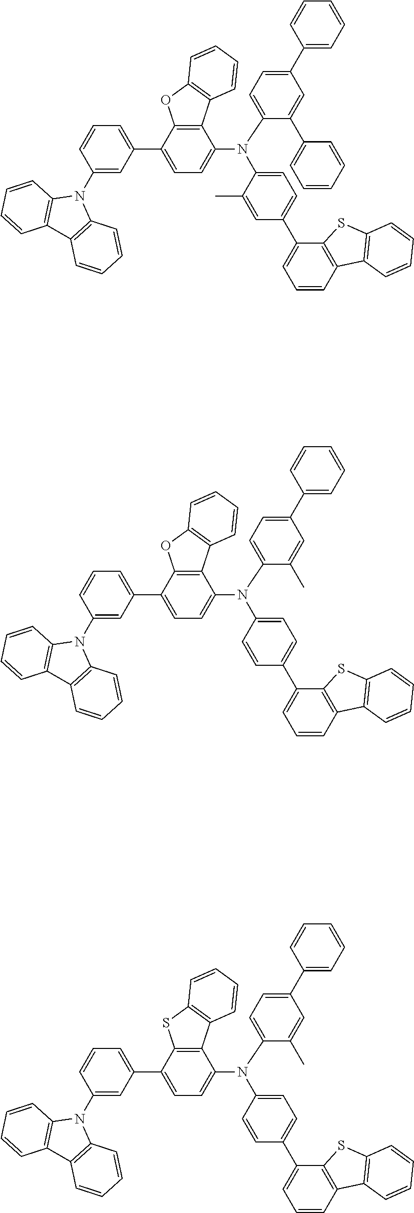 [SMILES: Cc1cc(-c2cccc3c2sc2ccccc23)ccc1N(c1ccc(-c2ccccc2)cc1-c1ccccc1)c1ccc(-c2cccc(-n3c4ccccc4c4ccccc43)c2)c2oc3ccccc3c12 ; Cc1cc(-c2ccccc2)ccc1N(c1ccc(-c2cccc3c2sc2ccccc23)cc1)c1ccc(-c2cccc(-n3c4ccccc4c4ccccc43)c2)c2oc3ccccc3c12 ; Cc1cc(-c2ccccc2)ccc1N(c1ccc(-c2cccc3c2sc2ccccc23)cc1)c1ccc(-c2cccc(-n3c4ccccc4c4ccccc43)c2)c2sc3ccccc3c12]

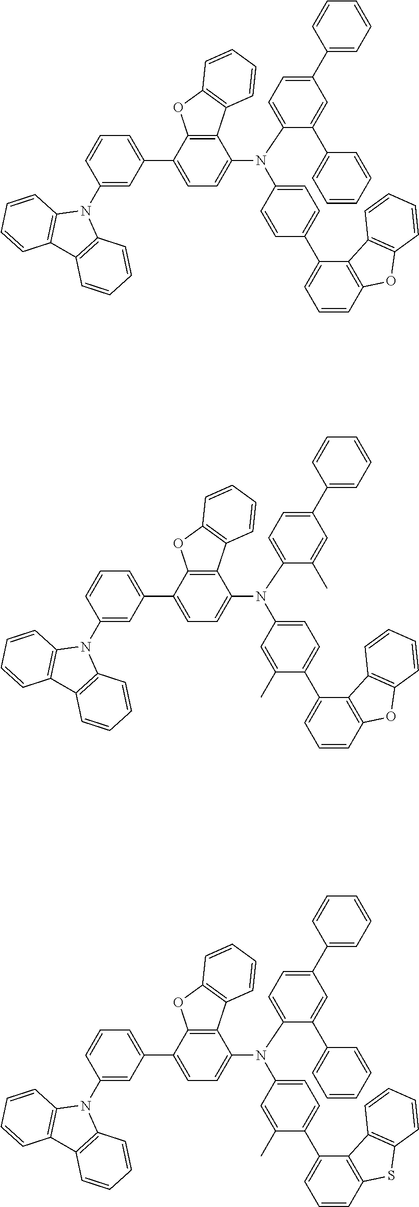 [SMILES: c1ccc(-c2ccc(N(c3ccc(-c4cccc5oc6ccccc6c45)cc3)c3ccc(-c4cccc(-n5c6ccccc6c6ccccc65)c4)c4oc5ccccc5c34)c(-c3ccccc3)c2)cc1 ; Cc1cc(N(c2ccc(-c3ccccc3)cc2C)c2ccc(-c3cccc(-n4c5ccccc5c5ccccc54)c3)c3oc4ccccc4c23)ccc1-c1cccc2oc3ccccc3c12 ; Cc1cc(N(c2ccc(-c3ccccc3)cc2-c2ccccc2)c2ccc(-c3cccc(-n4c5ccccc5c5ccccc54)c3)c3oc4ccccc4c23)ccc1-c1cccc2sc3ccccc3c12]

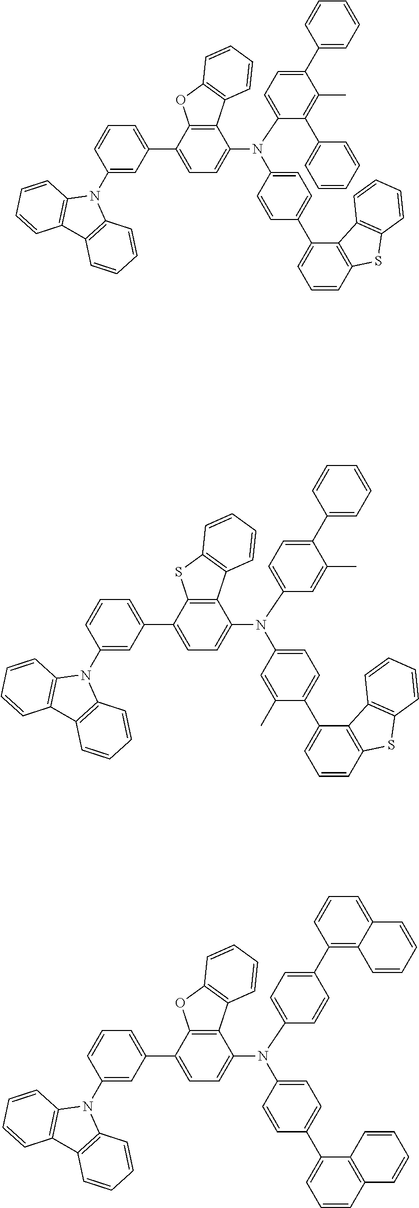 [SMILES: Cc1c(-c2ccccc2)ccc(N(c2ccc(-c3cccc4sc5ccccc5c34)cc2)c2ccc(-c3cccc(-n4c5ccccc5c5ccccc54)c3)c3oc4ccccc4c23)c1-c1ccccc1 ; Cc1cc(N(c2ccc(-c3cccc4sc5ccccc5c34)c(C)c2)c2ccc(-c3cccc(-n4c5ccccc5c5ccccc54)c3)c3sc4ccccc4c23)ccc1-c1ccccc1 ; c1cc(-c2ccc(N(c3ccc(-c4cccc5ccccc45)cc3)c3ccc(-c4cccc5ccccc45)cc3)c3c2oc2ccccc23)cc(-n2c3ccccc3c3ccccc32)c1]

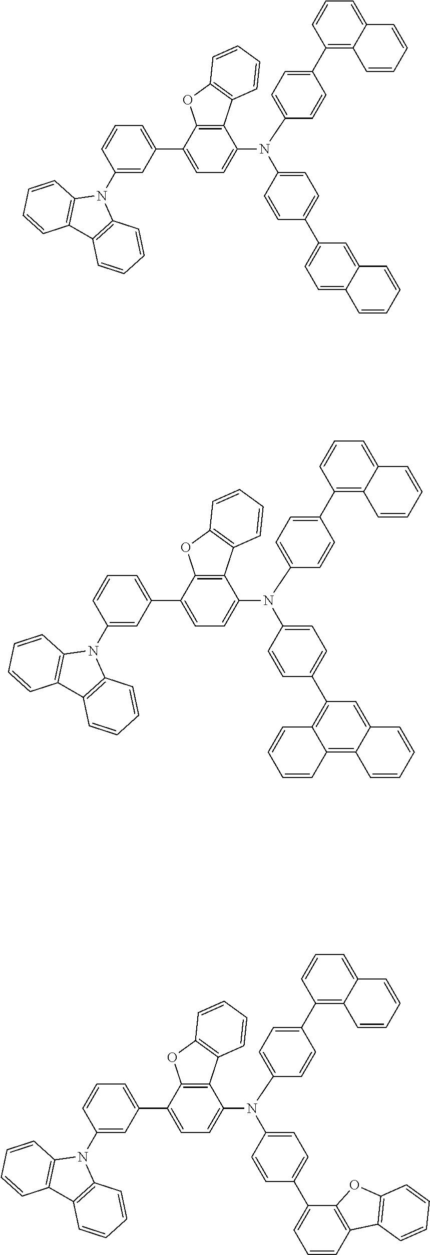 [SMILES: c1cc(-c2ccc(N(c3ccc(-c4ccc5ccccc5c4)cc3)c3ccc(-c4cccc5ccccc45)cc3)c3c2oc2ccccc23)cc(-n2c3ccccc3c3ccccc32)c1 ; c1cc(-c2ccc(N(c3ccc(-c4cccc5ccccc45)cc3)c3ccc(-c4cc5ccccc5c5ccccc45)cc3)c3c2oc2ccccc23)cc(-n2c3ccccc3c3ccccc32)c1 ; c1cc(-c2ccc(N(c3ccc(-c4cccc5ccccc45)cc3)c3ccc(-c4cccc5c4oc4ccccc45)cc3)c3c2oc2ccccc23)cc(-n2c3ccccc3c3ccccc32)c1]

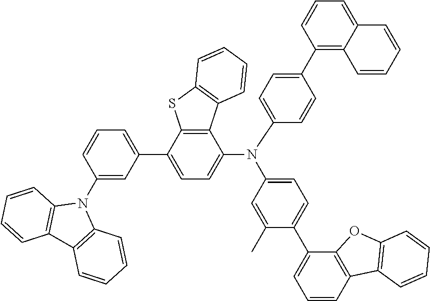 [SMILES: Cc1cc(N(c2ccc(-c3cccc4ccccc34)cc2)c2ccc(-c3cccc(-n4c5ccccc5c5ccccc54)c3)c3sc4ccccc4c23)ccc1-c1cccc2c1oc1ccccc12]

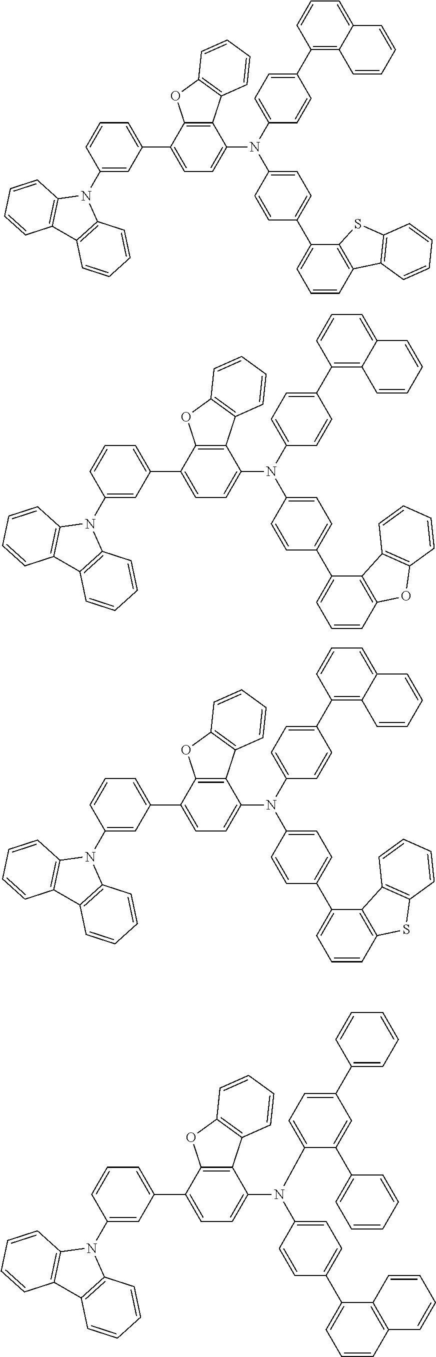

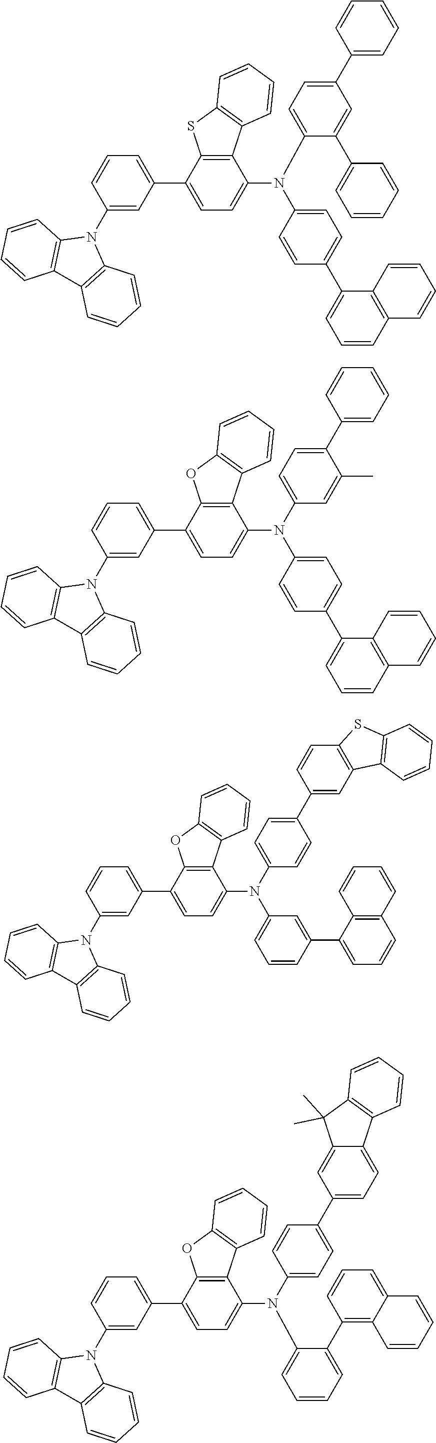

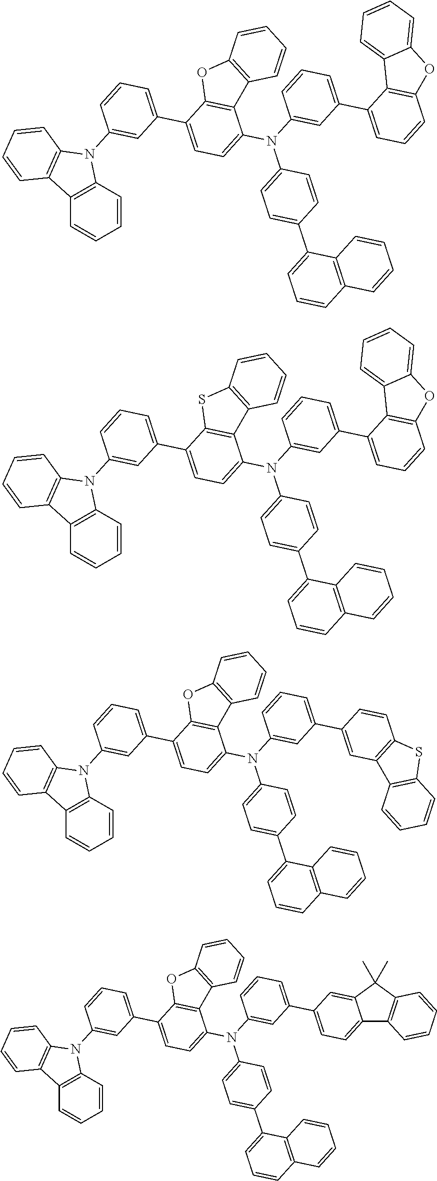 [SMILES: c1cc(-c2cccc3oc4ccccc4c23)cc(N(c2ccc(-c3cccc4ccccc34)cc2)c2ccc(-c3cccc(-n4c5ccccc5c5ccccc54)c3)c3oc4ccccc4c23)c1 ; c1cc(-c2cccc3oc4ccccc4c23)cc(N(c2ccc(-c3cccc4ccccc34)cc2)c2ccc(-c3cccc(-n4c5ccccc5c5ccccc54)c3)c3sc4ccccc4c23)c1 ; c1cc(-c2ccc3sc4ccccc4c3c2)cc(N(c2ccc(-c3cccc4ccccc34)cc2)c2ccc(-c3cccc(-n4c5ccccc5c5ccccc54)c3)c3oc4ccccc4c23)c1 ; CC1(C)c2ccccc2-c2ccc(-c3cccc(N(c4ccc(-c5cccc6ccccc56)cc4)c4ccc(-c5cccc(-n6c7ccccc7c7ccccc76)c5)c5oc6ccccc6c45)c3)cc21]

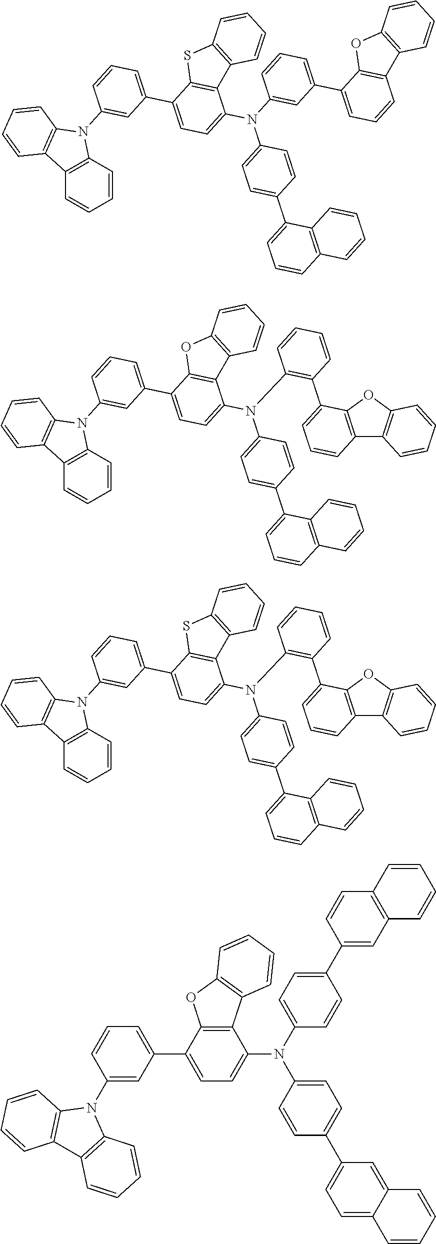

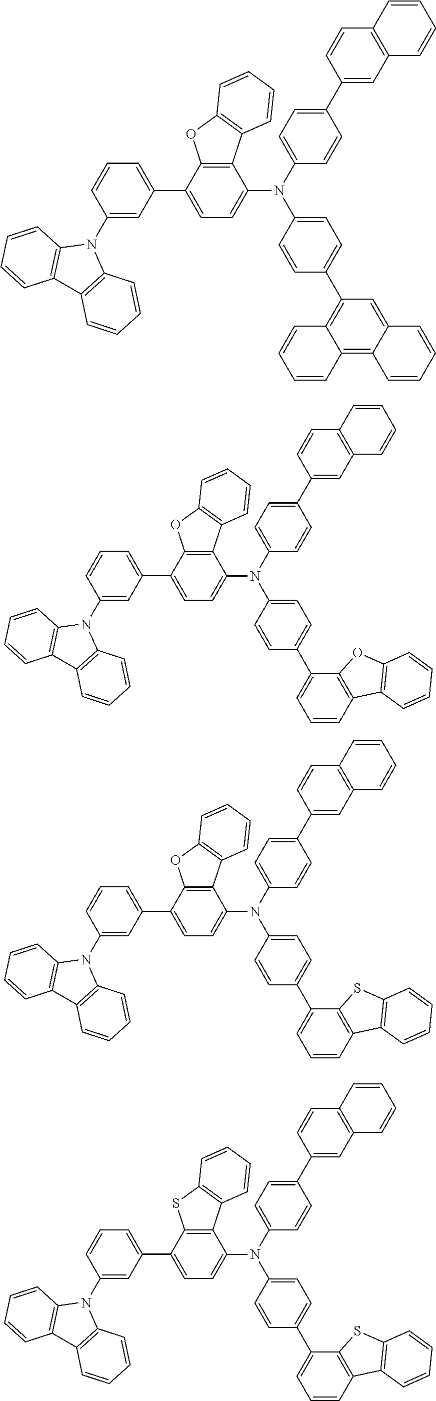

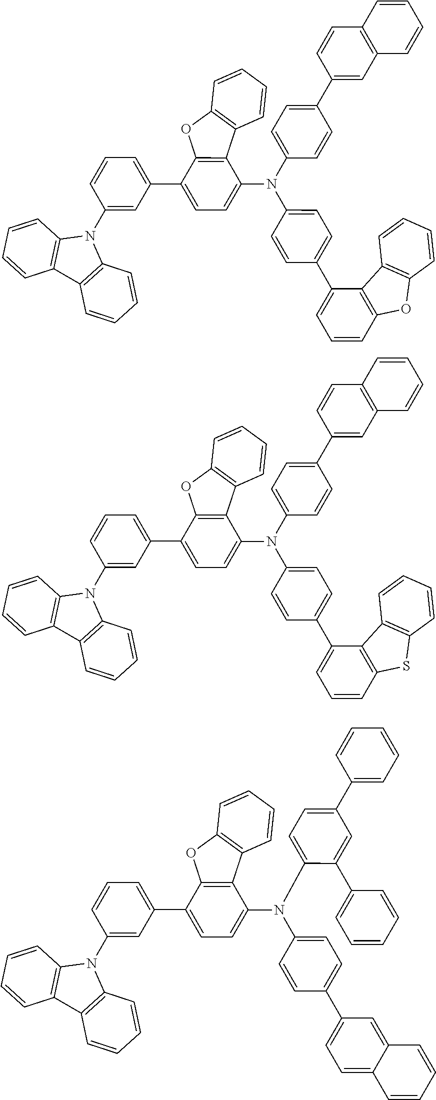

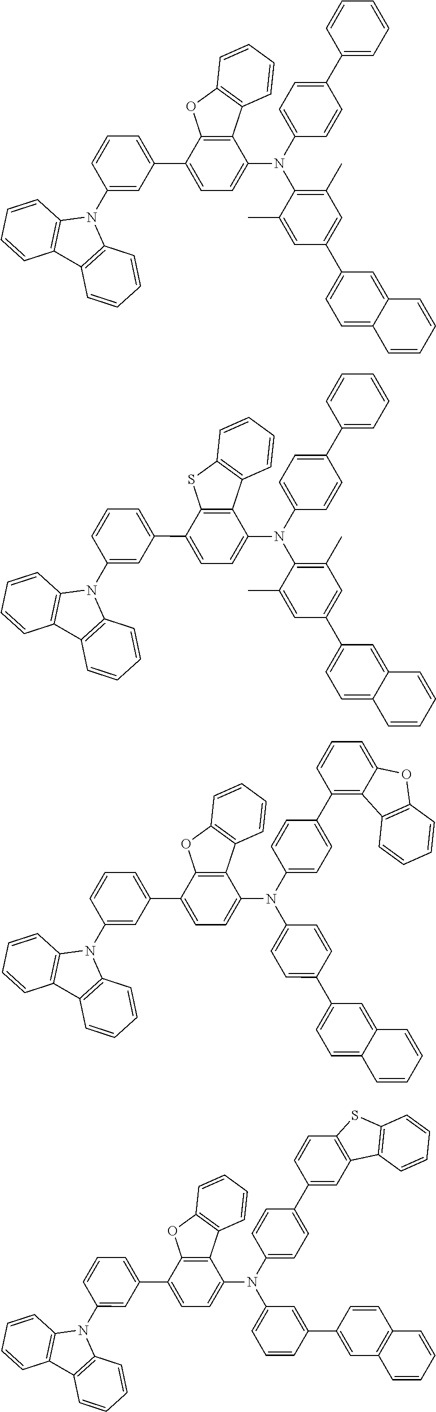

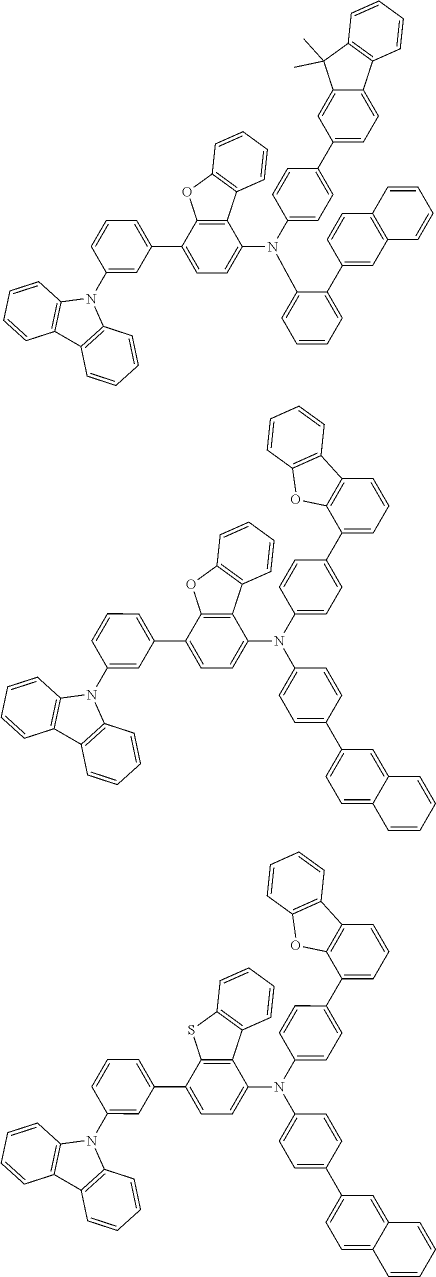 [SMILES: CC1(C)c2ccccc2-c2ccc(-c3ccc(N(c4ccccc4-c4ccc5ccccc5c4)c4ccc(-c5cccc(-n6c7ccccc7c7ccccc76)c5)c5oc6ccccc6c45)cc3)cc21 ; c1cc(-c2ccc(N(c3ccc(-c4ccc5ccccc5c4)cc3)c3ccc(-c4cccc5c4oc4ccccc45)cc3)c3c2oc2ccccc23)cc(-n2c3ccccc3c3ccccc32)c1 ; c1cc(-c2ccc(N(c3ccc(-c4ccc5ccccc5c4)cc3)c3ccc(-c4cccc5c4oc4ccccc45)cc3)c3c2sc2ccccc23)cc(-n2c3ccccc3c3ccccc32)c1]

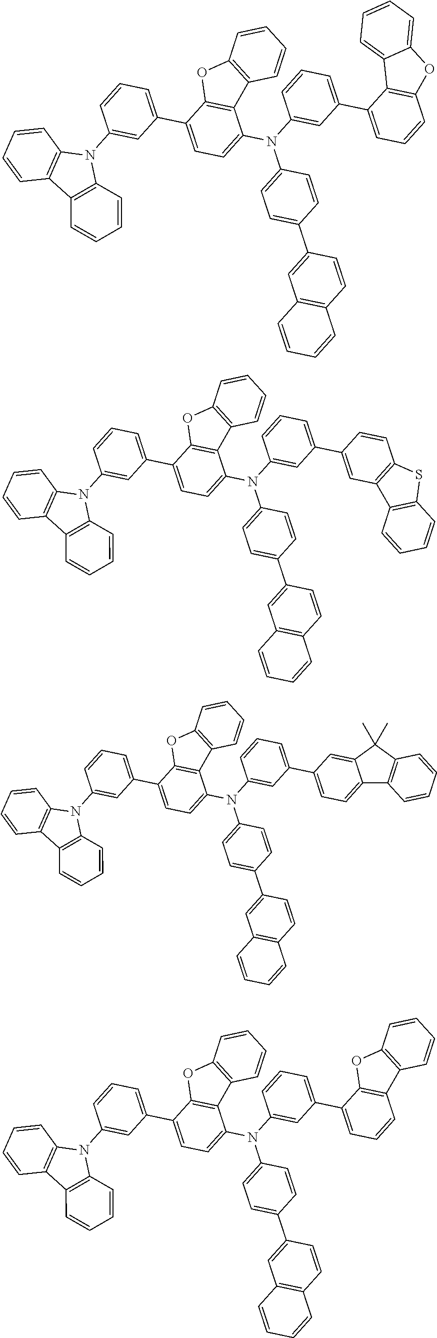 [SMILES: c1cc(-c2cccc3oc4ccccc4c23)cc(N(c2ccc(-c3ccc4ccccc4c3)cc2)c2ccc(-c3cccc(-n4c5ccccc5c5ccccc54)c3)c3oc4ccccc4c23)c1 ; c1cc(-c2ccc3sc4ccccc4c3c2)cc(N(c2ccc(-c3ccc4ccccc4c3)cc2)c2ccc(-c3cccc(-n4c5ccccc5c5ccccc54)c3)c3oc4ccccc4c23)c1 ; CC1(C)c2ccccc2-c2ccc(-c3cccc(N(c4ccc(-c5ccc6ccccc6c5)cc4)c4ccc(-c5cccc(-n6c7ccccc7c7ccccc76)c5)c5oc6ccccc6c45)c3)cc21 ; c1cc(-c2cccc3c2oc2ccccc23)cc(N(c2ccc(-c3ccc4ccccc4c3)cc2)c2ccc(-c3cccc(-n4c5ccccc5c5ccccc54)c3)c3oc4ccccc4c23)c1]

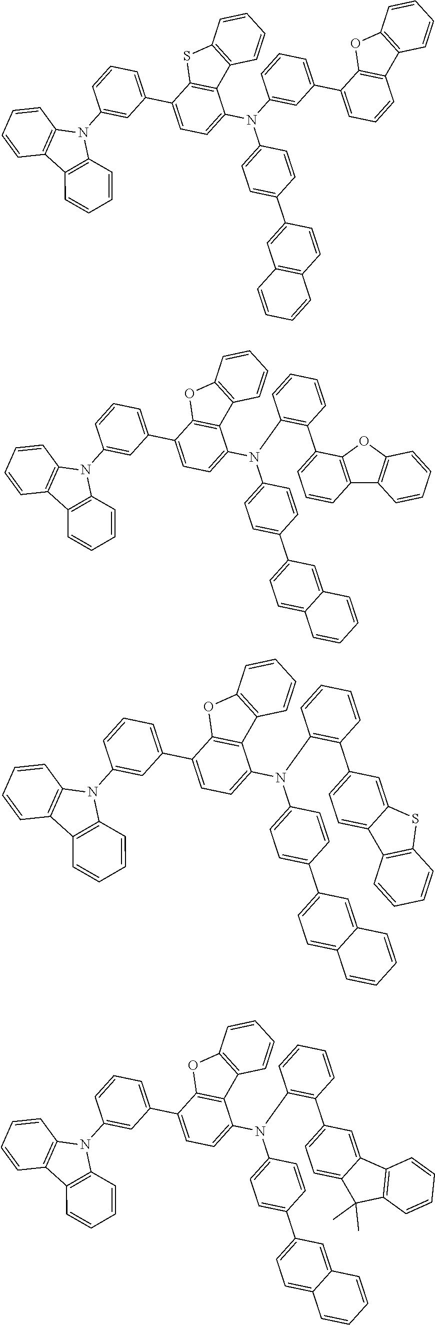 [SMILES: c1cc(-c2cccc3c2oc2ccccc23)cc(N(c2ccc(-c3ccc4ccccc4c3)cc2)c2ccc(-c3cccc(-n4c5ccccc5c5ccccc54)c3)c3sc4ccccc4c23)c1 ; c1cc(-c2ccc(N(c3ccc(-c4ccc5ccccc5c4)cc3)c3ccccc3-c3cccc4c3oc3ccccc34)c3c2oc2ccccc23)cc(-n2c3ccccc3c3ccccc32)c1 ; c1cc(-c2ccc(N(c3ccc(-c4ccc5ccccc5c4)cc3)c3ccccc3-c3ccc4c(c3)sc3ccccc34)c3c2oc2ccccc23)cc(-n2c3ccccc3c3ccccc32)c1 ; CC1(C)c2ccccc2-c2cc(-c3ccccc3N(c3ccc(-c4ccc5ccccc5c4)cc3)c3ccc(-c4cccc(-n5c6ccccc6c6ccccc65)c4)c4oc5ccccc5c34)ccc21]

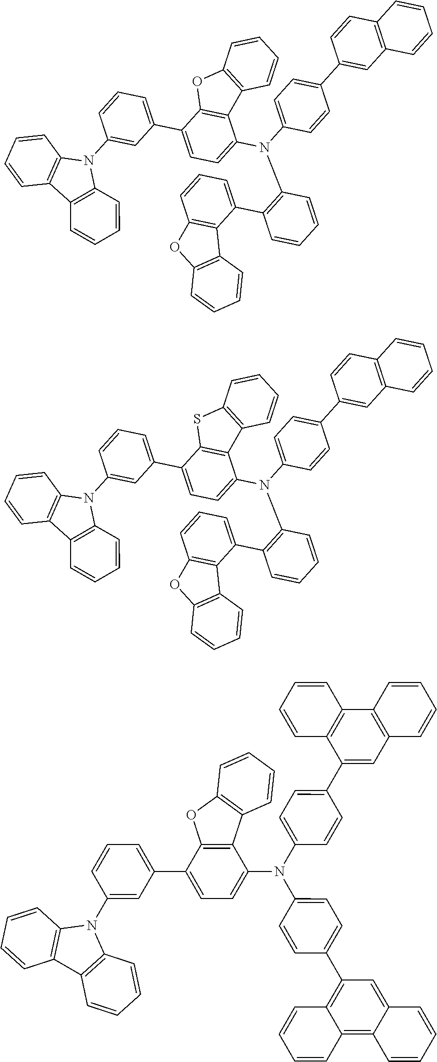 [SMILES: c1cc(-c2ccc(N(c3ccc(-c4ccc5ccccc5c4)cc3)c3ccccc3-c3cccc4oc5ccccc5c34)c3c2oc2ccccc23)cc(-n2c3ccccc3c3ccccc32)c1 ; c1cc(-c2ccc(N(c3ccc(-c4ccc5ccccc5c4)cc3)c3ccccc3-c3cccc4oc5ccccc5c34)c3c2sc2ccccc23)cc(-n2c3ccccc3c3ccccc32)c1 ; c1cc(-c2ccc(N(c3ccc(-c4cc5ccccc5c5ccccc45)cc3)c3ccc(-c4cc5ccccc5c5ccccc45)cc3)c3c2oc2ccccc23)cc(-n2c3ccccc3c3ccccc32)c1]

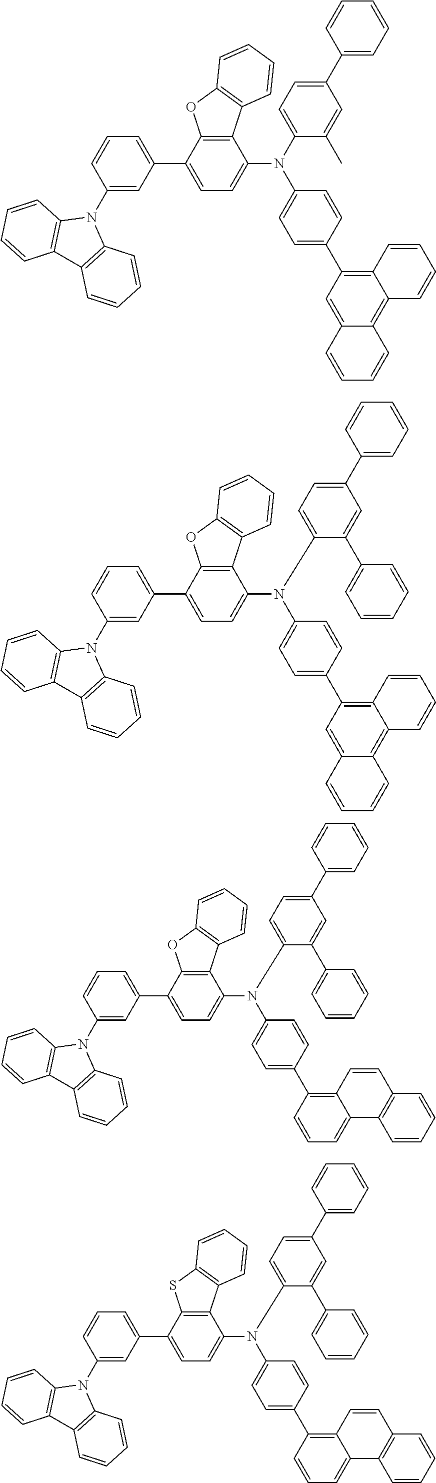

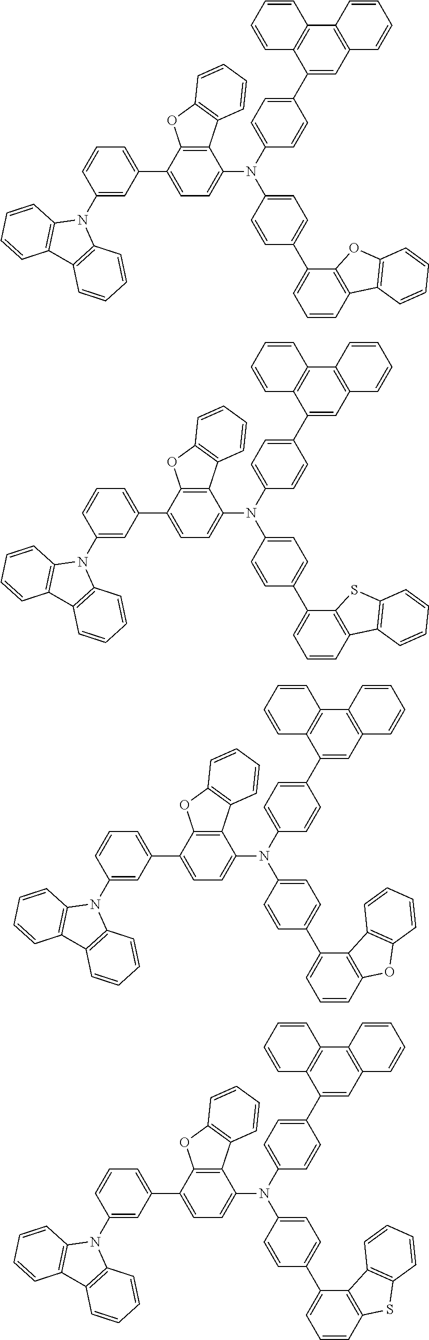

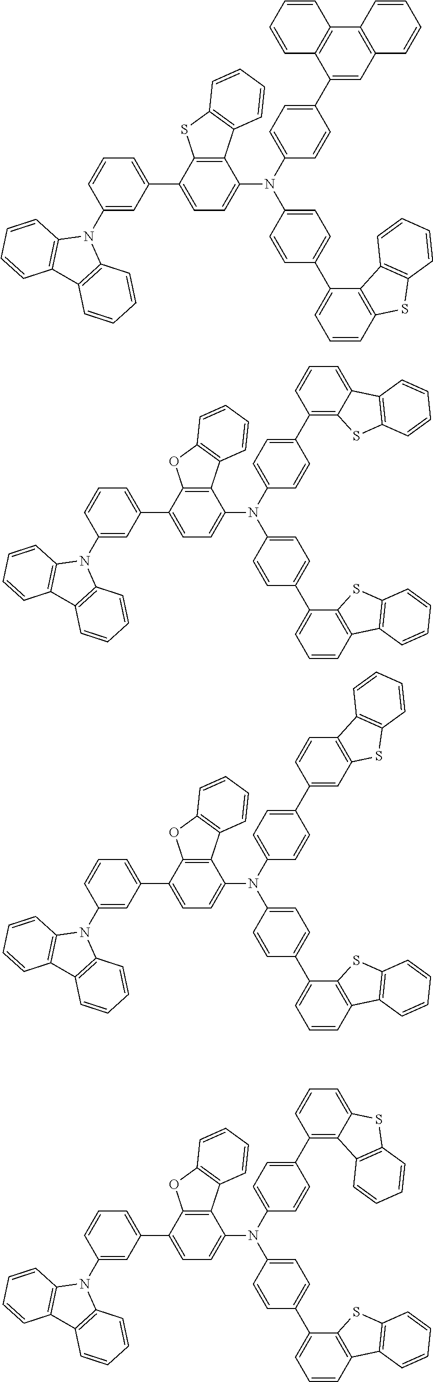

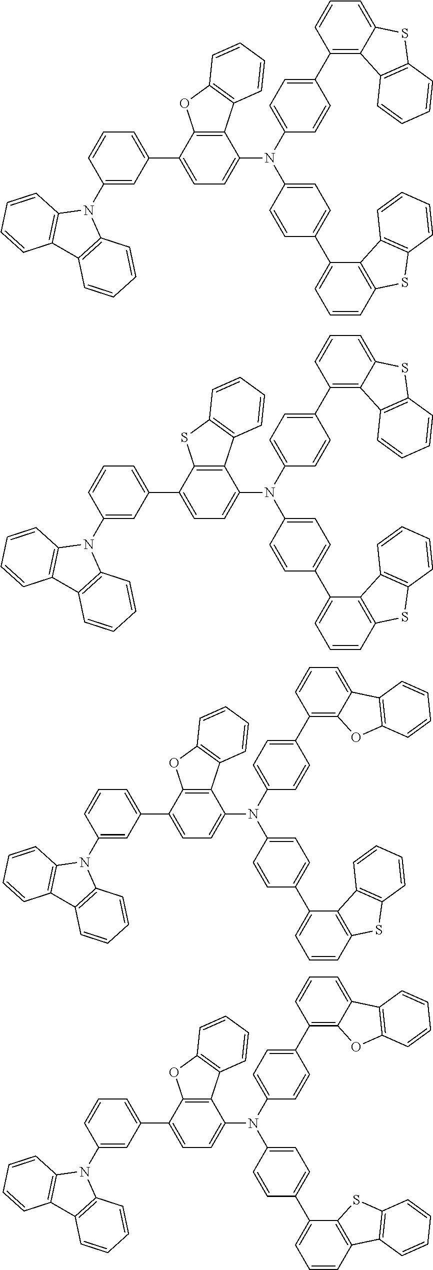

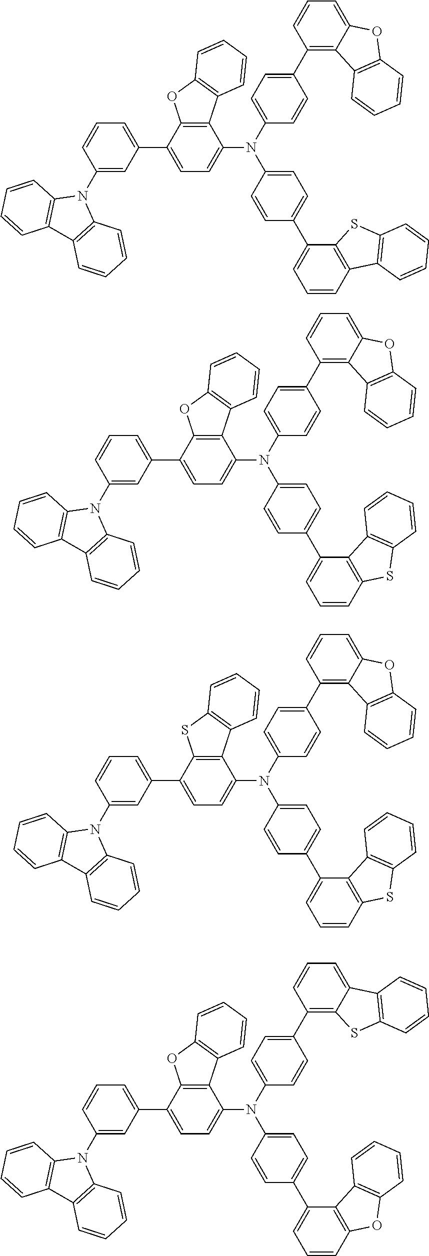

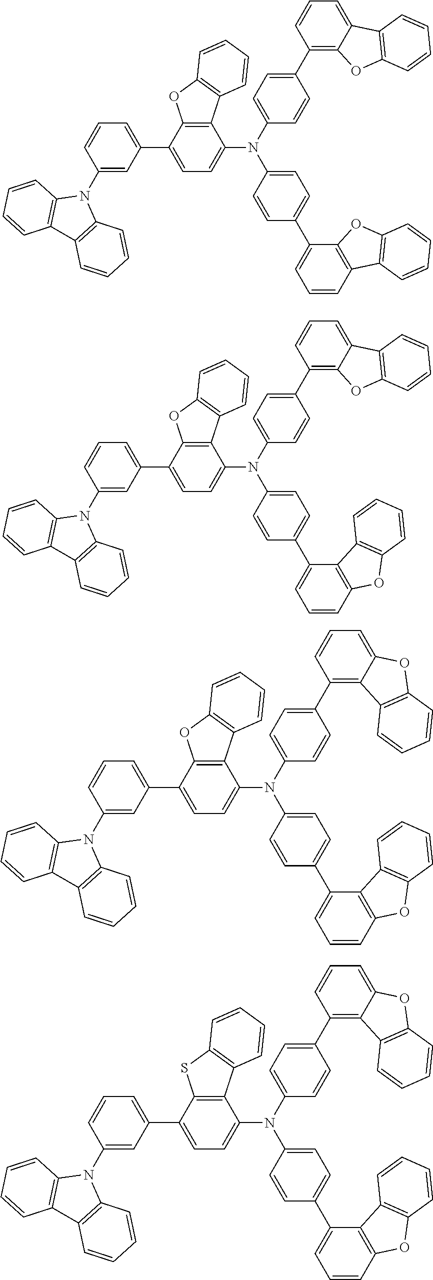

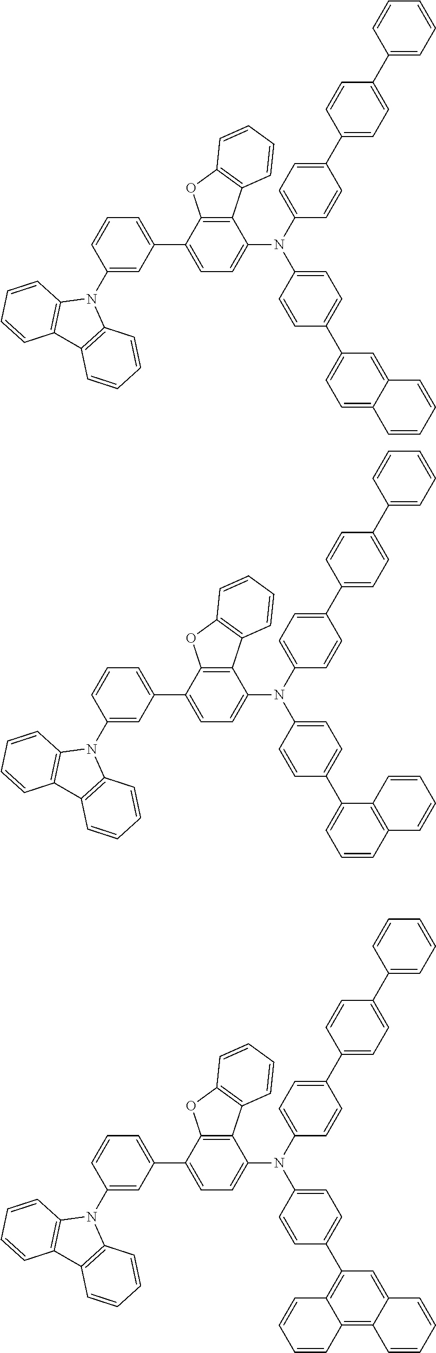

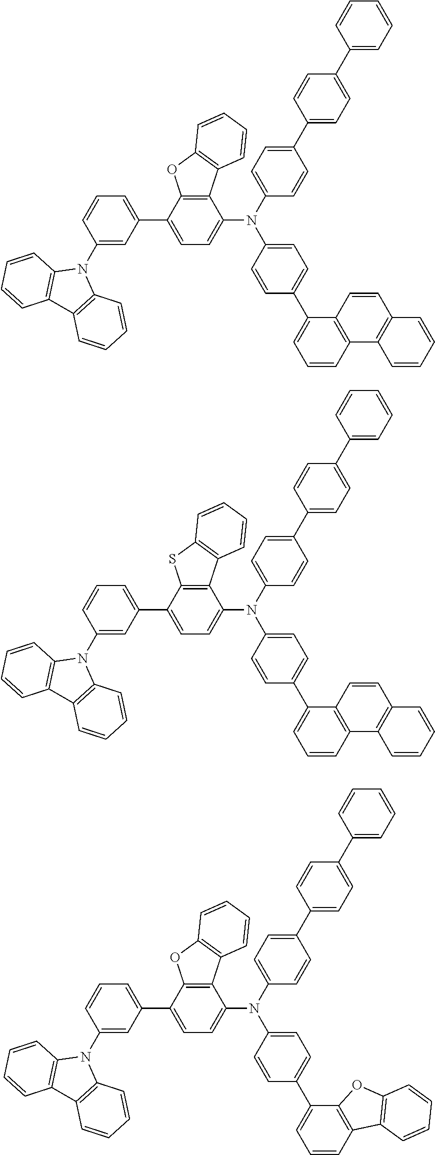

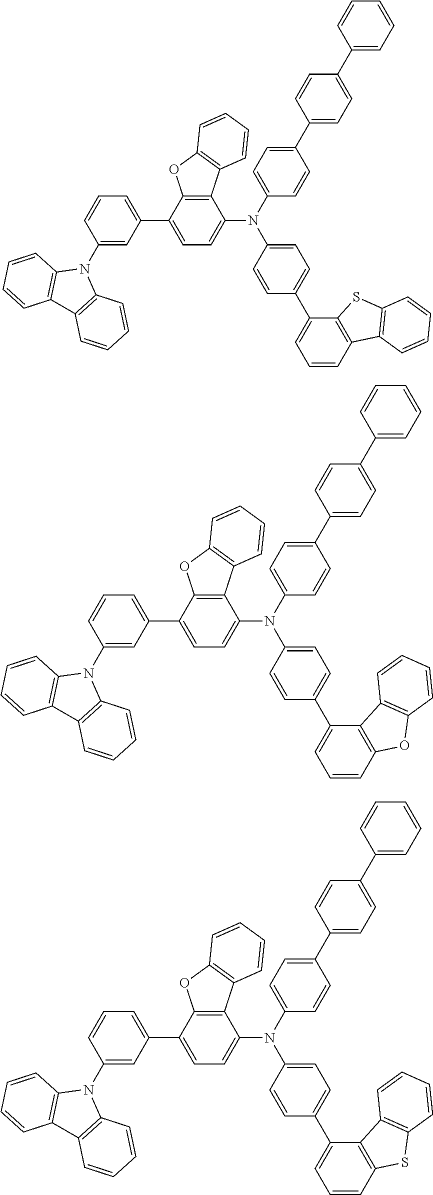

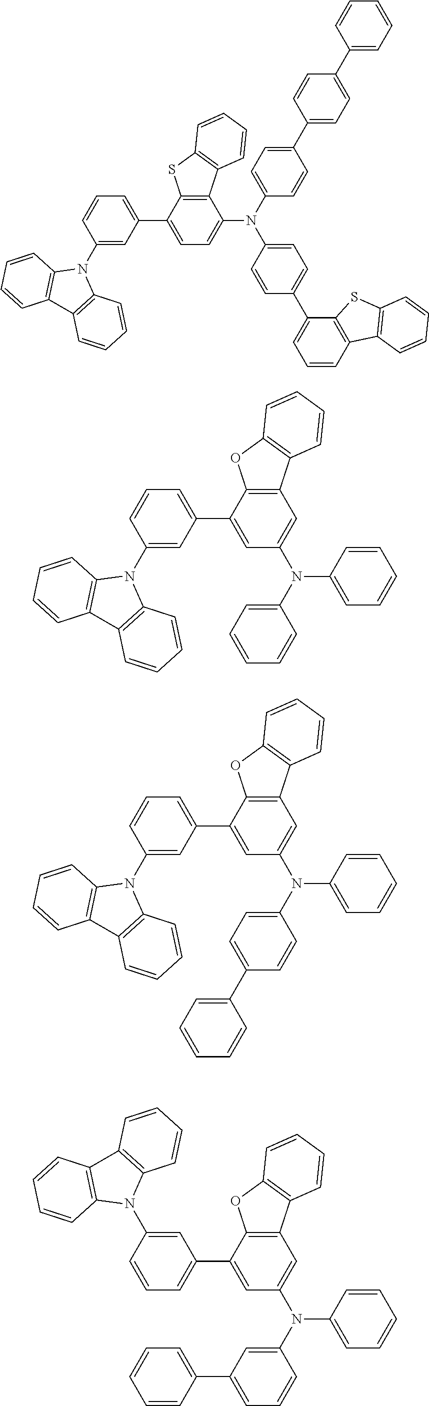

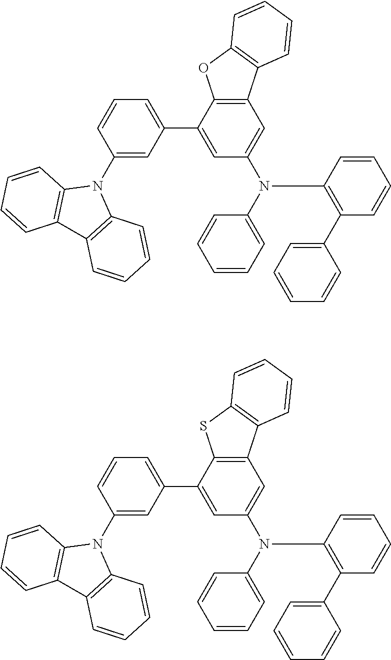 [SMILES: c1ccc(-c2ccccc2N(c2ccccc2)c2cc(-c3cccc(-n4c5ccccc5c5ccccc54)c3)c3oc4ccccc4c3c2)cc1 ; c1ccc(-c2ccccc2N(c2ccccc2)c2cc(-c3cccc(-n4c5ccccc5c5ccccc54)c3)c3sc4ccccc4c3c2)cc1]

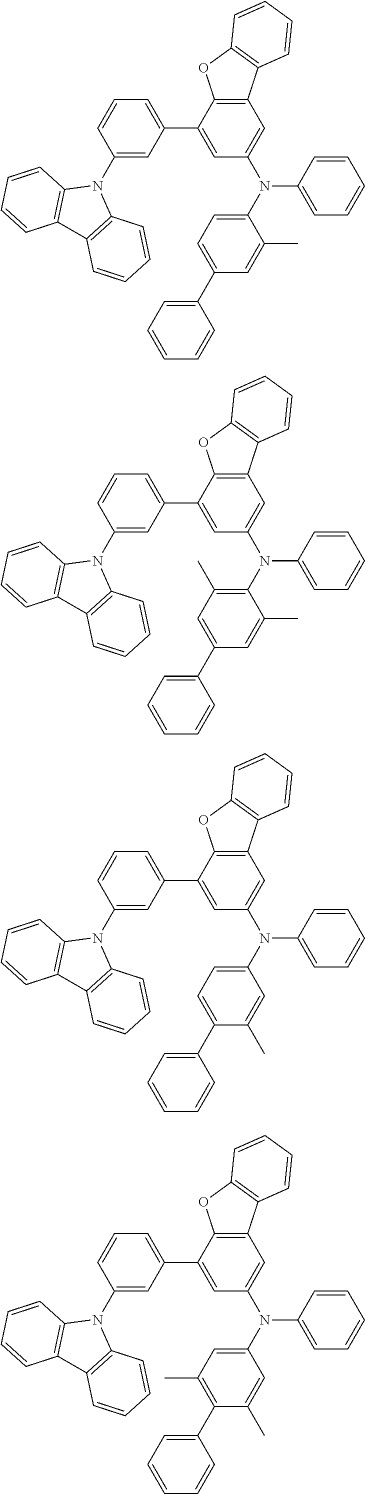 [SMILES: Cc1cc(-c2ccccc2)ccc1N(c1ccccc1)c1cc(-c2cccc(-n3c4ccccc4c4ccccc43)c2)c2oc3ccccc3c2c1 ; Cc1cc(-c2ccccc2)cc(C)c1N(c1ccccc1)c1cc(-c2cccc(-n3c4ccccc4c4ccccc43)c2)c2oc3ccccc3c2c1 ; Cc1cc(N(c2ccccc2)c2cc(-c3cccc(-n4c5ccccc5c5ccccc54)c3)c3oc4ccccc4c3c2)ccc1-c1ccccc1 ; Cc1cc(N(c2ccccc2)c2cc(-c3cccc(-n4c5ccccc5c5ccccc54)c3)c3oc4ccccc4c3c2)cc(C)c1-c1ccccc1]

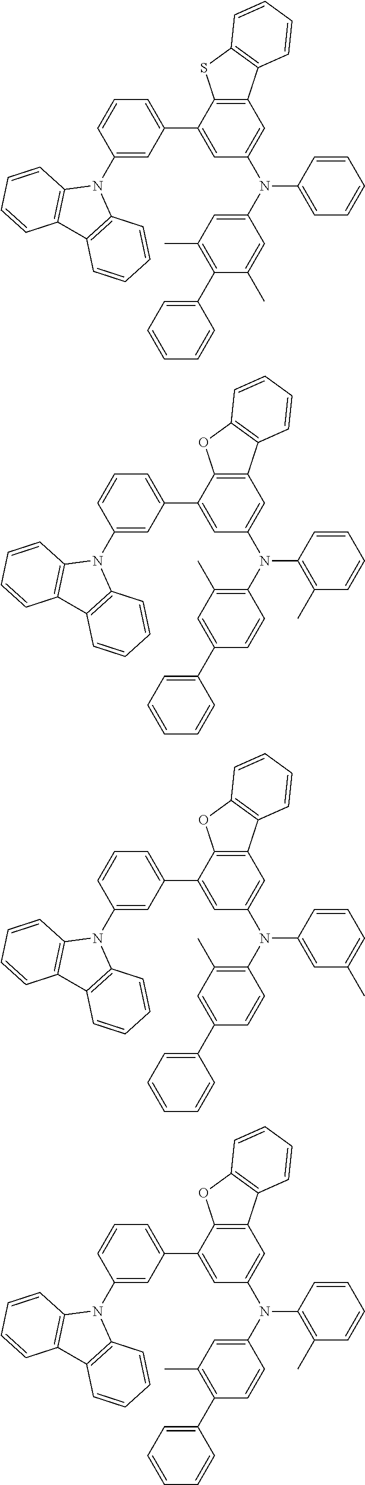 [SMILES: Cc1cc(N(c2ccccc2)c2cc(-c3cccc(-n4c5ccccc5c5ccccc54)c3)c3sc4ccccc4c3c2)cc(C)c1-c1ccccc1 ; Cc1ccccc1N(c1cc(-c2cccc(-n3c4ccccc4c4ccccc43)c2)c2oc3ccccc3c2c1)c1ccc(-c2ccccc2)cc1C ; Cc1cccc(N(c2cc(-c3cccc(-n4c5ccccc5c5ccccc54)c3)c3oc4ccccc4c3c2)c2ccc(-c3ccccc3)cc2C)c1 ; Cc1cc(N(c2cc(-c3cccc(-n4c5ccccc5c5ccccc54)c3)c3oc4ccccc4c3c2)c2ccccc2C)ccc1-c1ccccc1]

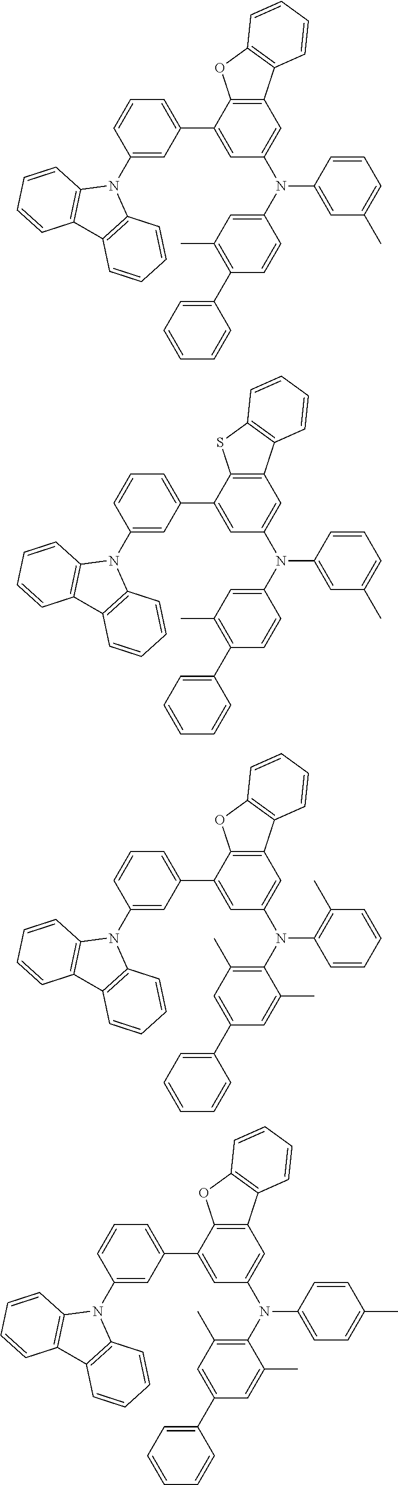

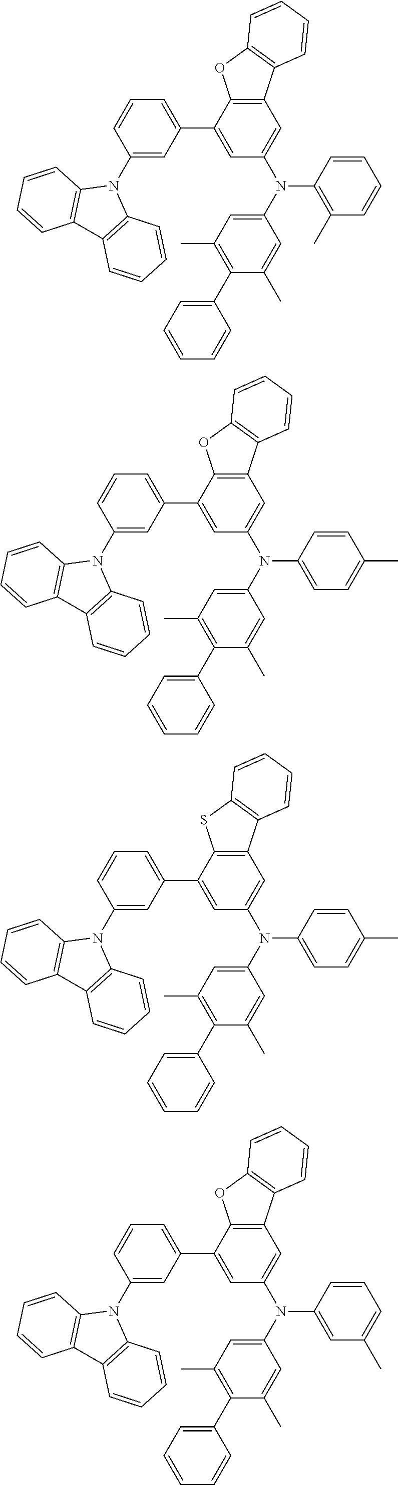

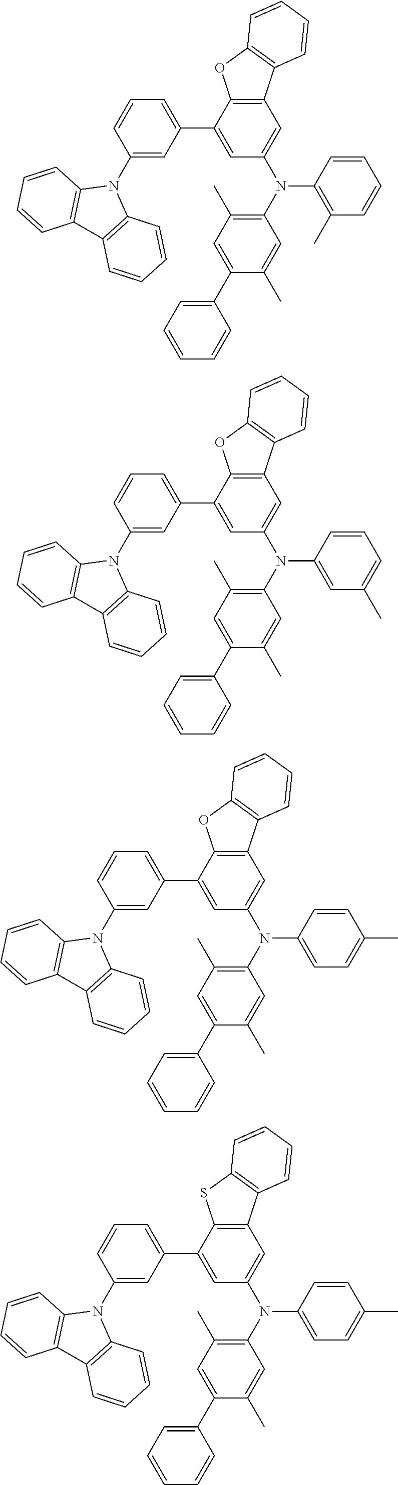

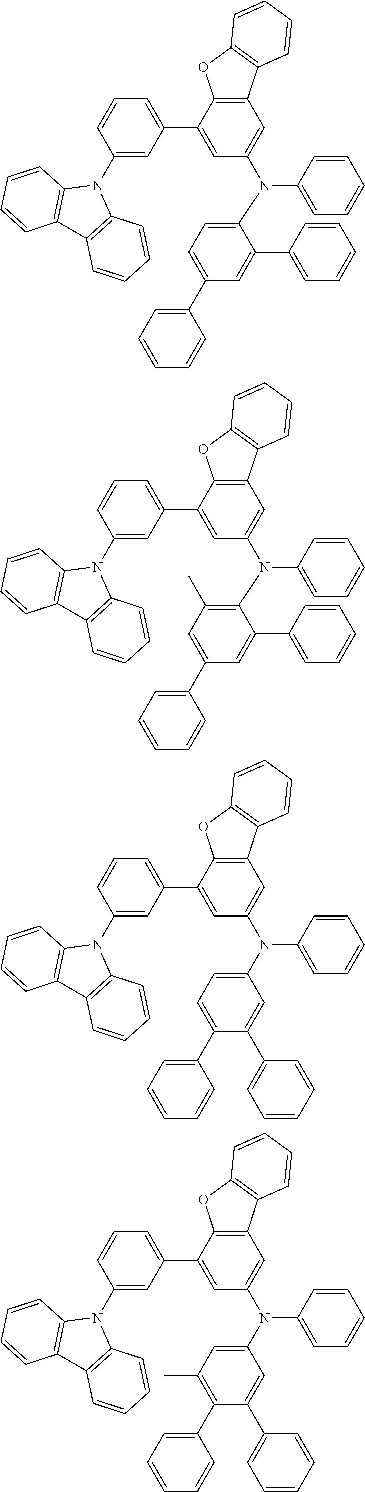

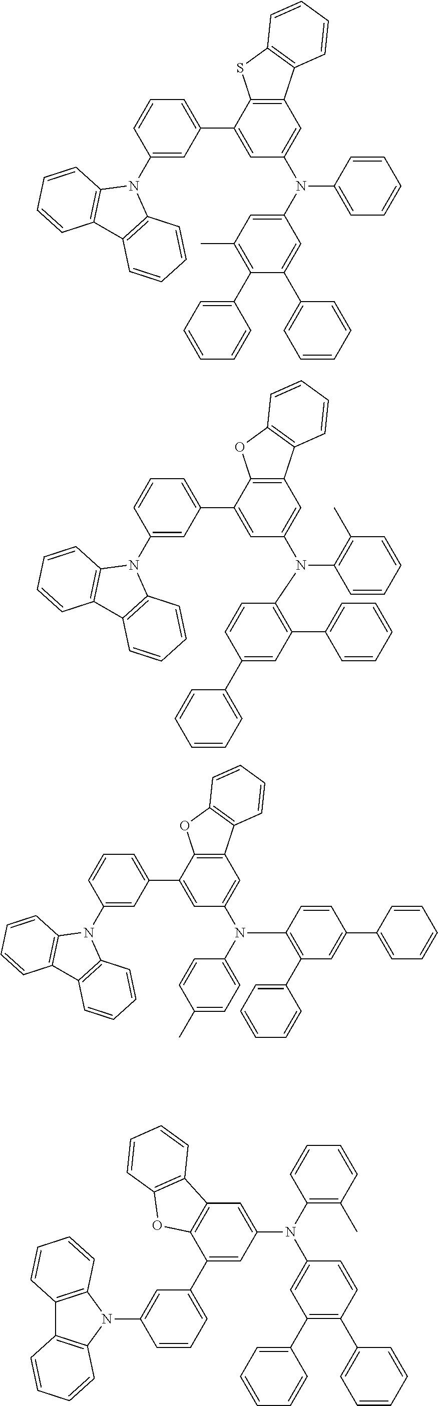

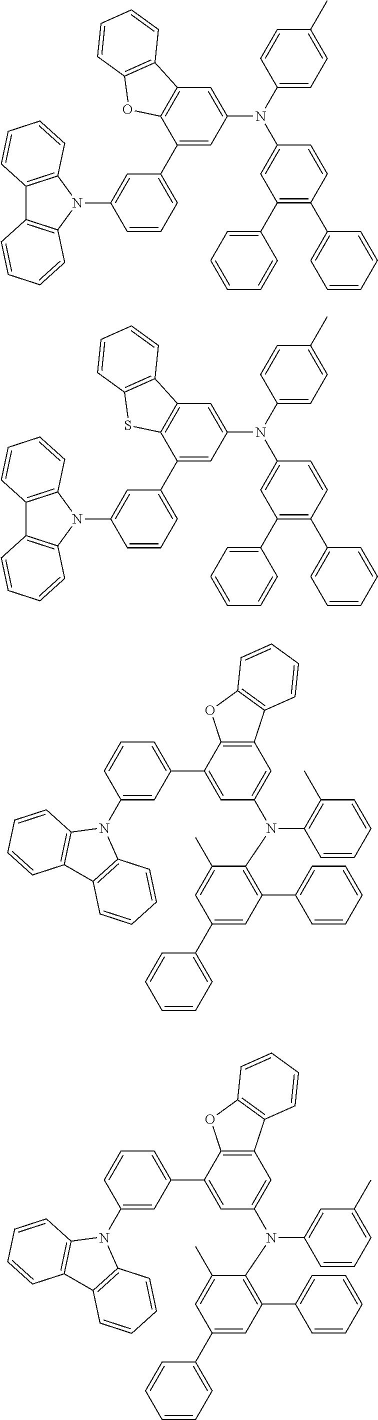

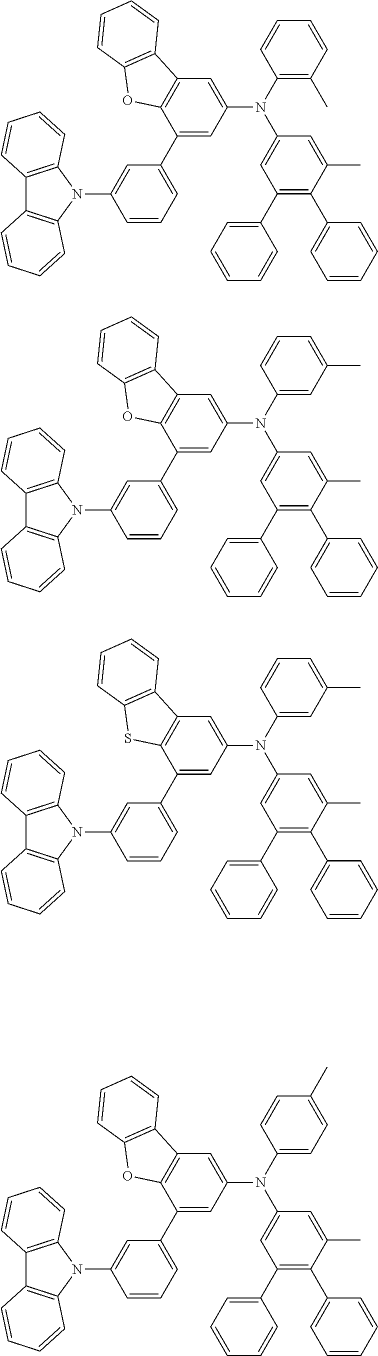 [SMILES: Cc1ccccc1N(c1cc(C)c(-c2ccccc2)c(-c2ccccc2)c1)c1cc(-c2cccc(-n3c4ccccc4c4ccccc43)c2)c2oc3ccccc3c2c1 ; Cc1cccc(N(c2cc(C)c(-c3ccccc3)c(-c3ccccc3)c2)c2cc(-c3cccc(-n4c5ccccc5c5ccccc54)c3)c3oc4ccccc4c3c2)c1 ; Cc1cccc(N(c2cc(C)c(-c3ccccc3)c(-c3ccccc3)c2)c2cc(-c3cccc(-n4c5ccccc5c5ccccc54)c3)c3sc4ccccc4c3c2)c1 ; Cc1ccc(N(c2cc(C)c(-c3ccccc3)c(-c3ccccc3)c2)c2cc(-c3cccc(-n4c5ccccc5c5ccccc54)c3)c3oc4ccccc4c3c2)cc1]

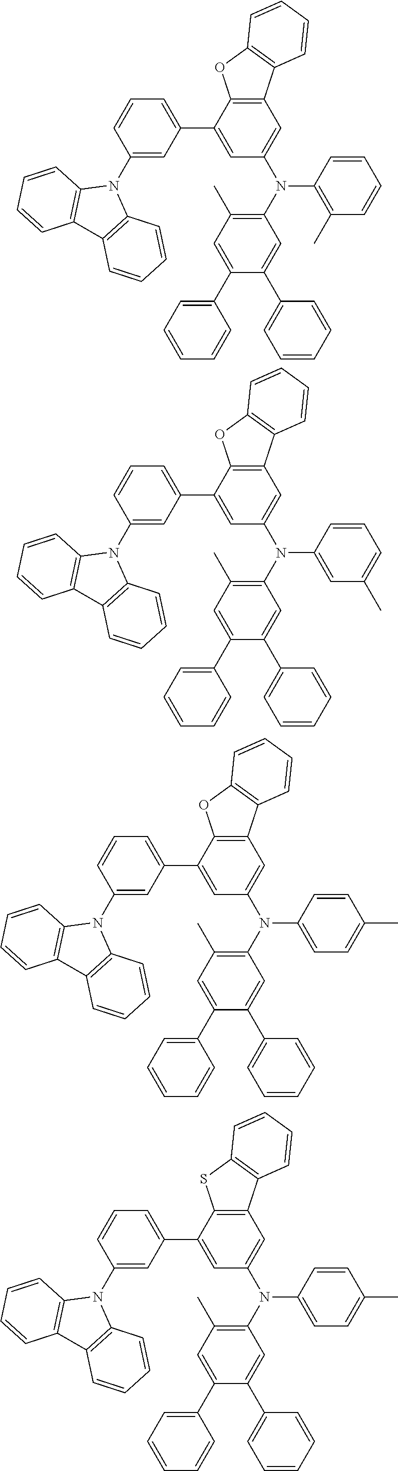

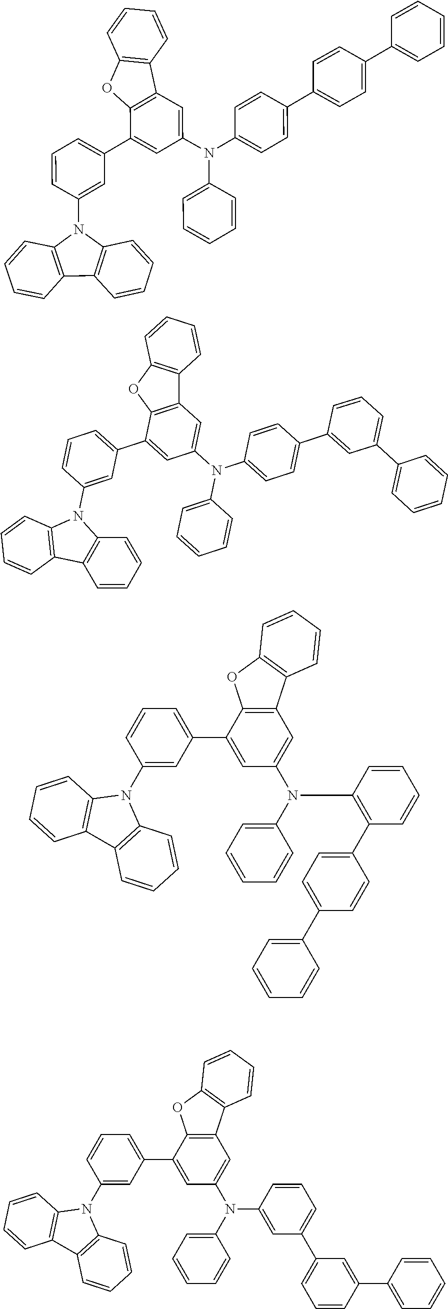

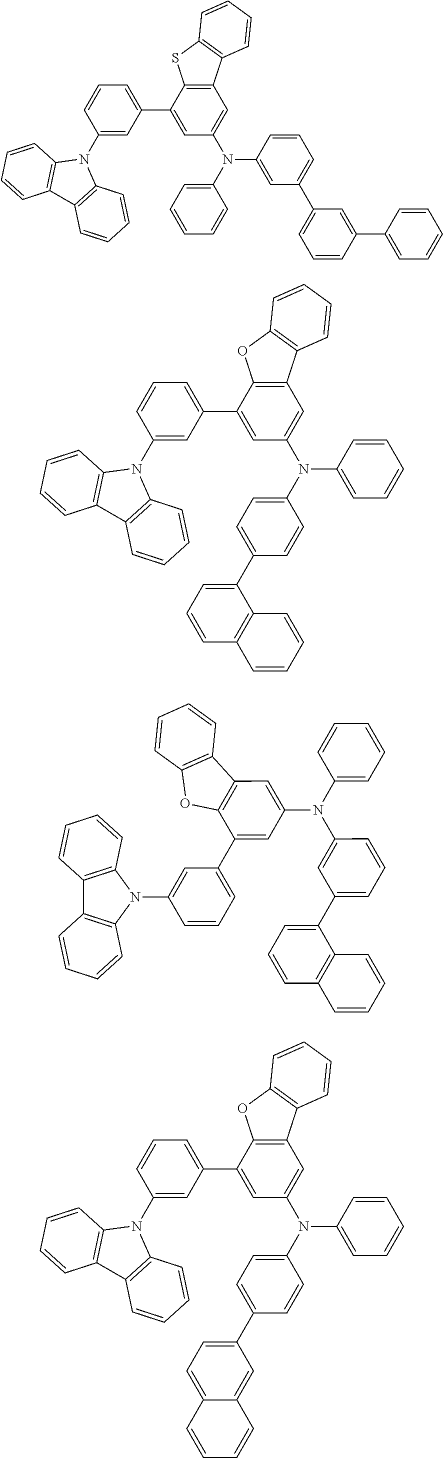

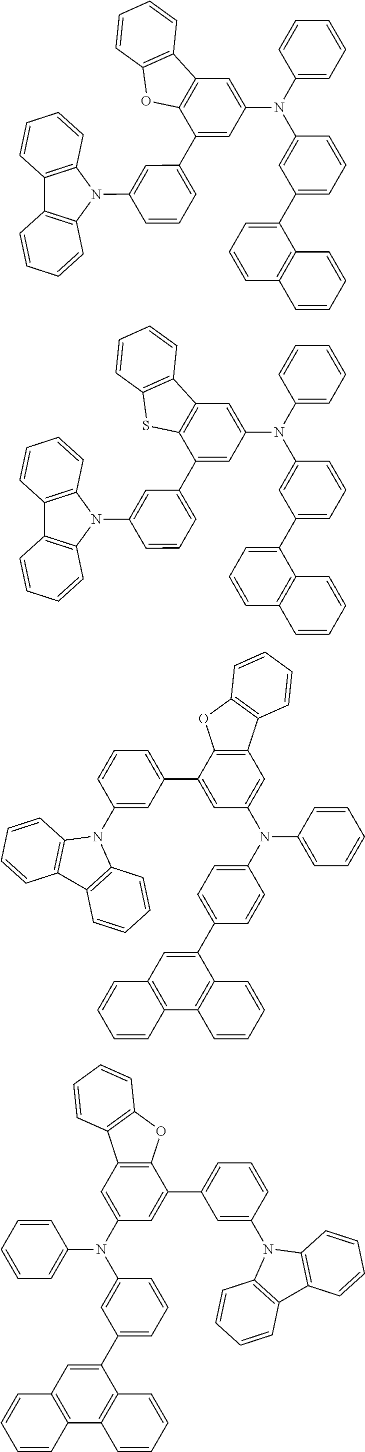

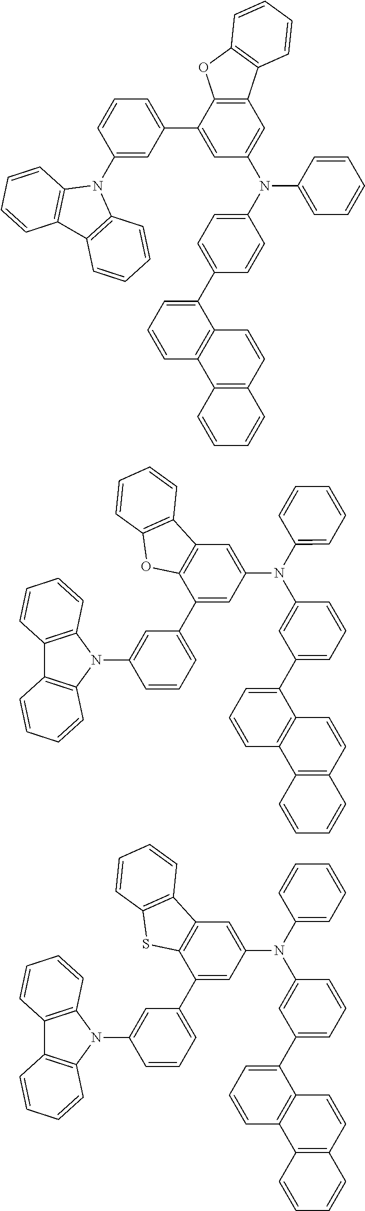

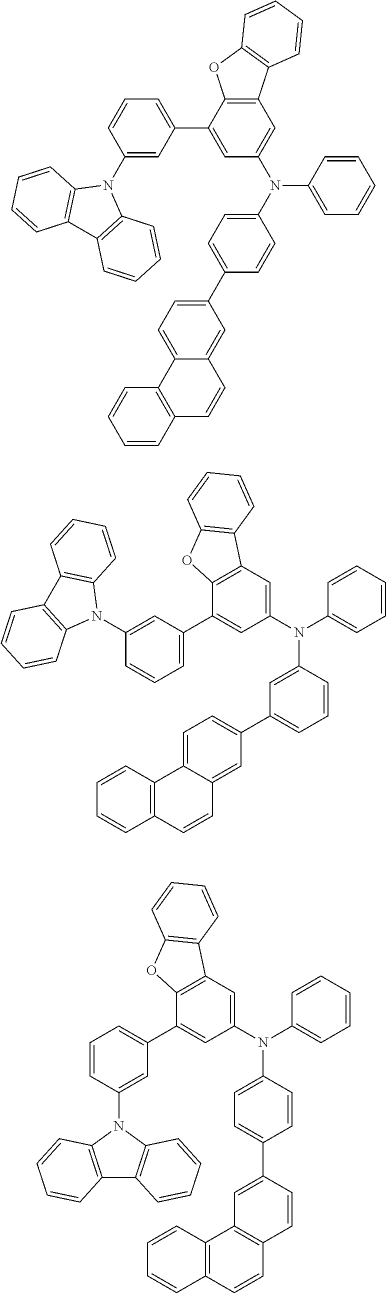 [SMILES: c1ccc(N(c2ccc(-c3ccc4c(ccc5ccccc54)c3)cc2)c2cc(-c3cccc(-n4c5ccccc5c5ccccc54)c3)c3oc4ccccc4c3c2)cc1 ; c1ccc(N(c2cccc(-c3ccc4c(ccc5ccccc54)c3)c2)c2cc(-c3cccc(-n4c5ccccc5c5ccccc54)c3)c3oc4ccccc4c3c2)cc1 ; c1ccc(N(c2ccc(-c3ccc4ccc5ccccc5c4c3)cc2)c2cc(-c3cccc(-n4c5ccccc5c5ccccc54)c3)c3oc4ccccc4c3c2)cc1]

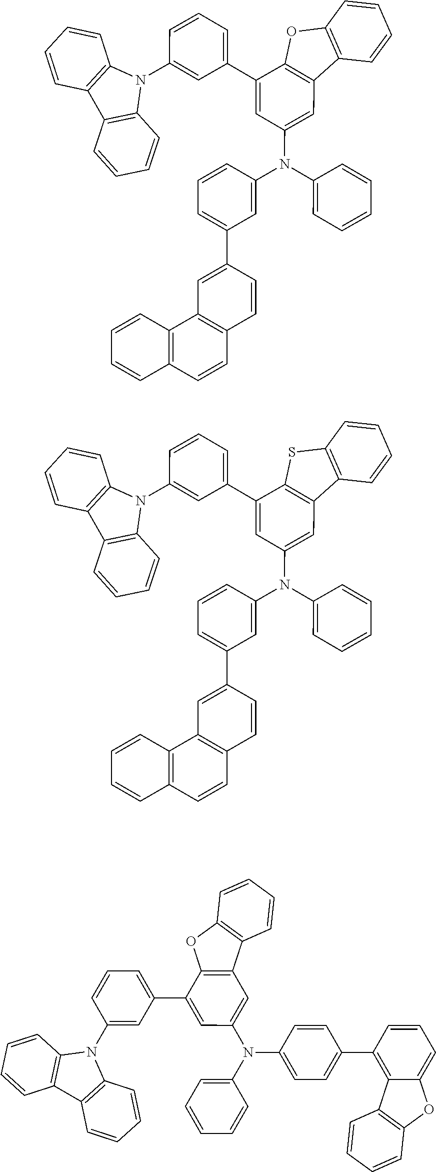 [SMILES: c1ccc(N(c2cccc(-c3ccc4ccc5ccccc5c4c3)c2)c2cc(-c3cccc(-n4c5ccccc5c5ccccc54)c3)c3oc4ccccc4c3c2)cc1 ; c1ccc(N(c2cccc(-c3ccc4ccc5ccccc5c4c3)c2)c2cc(-c3cccc(-n4c5ccccc5c5ccccc54)c3)c3sc4ccccc4c3c2)cc1 ; c1ccc(N(c2ccc(-c3cccc4oc5ccccc5c34)cc2)c2cc(-c3cccc(-n4c5ccccc5c5ccccc54)c3)c3oc4ccccc4c3c2)cc1]

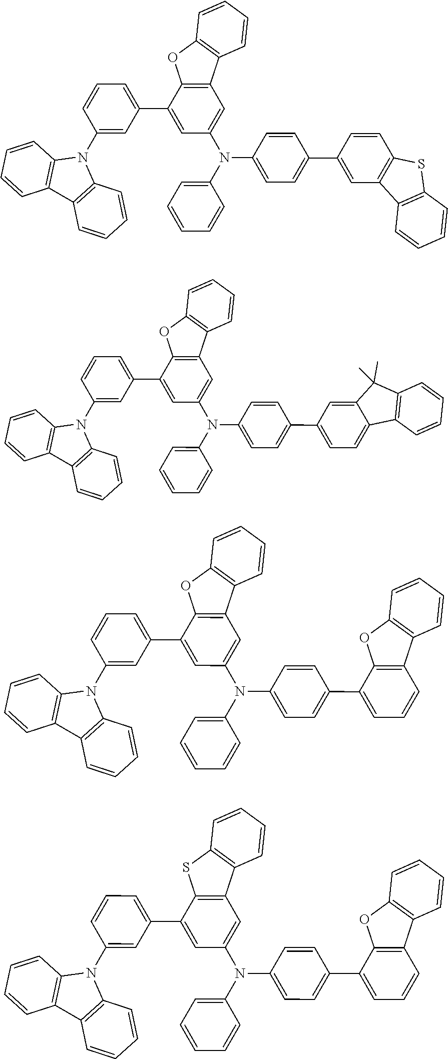 [SMILES: c1ccc(N(c2ccc(-c3ccc4sc5ccccc5c4c3)cc2)c2cc(-c3cccc(-n4c5ccccc5c5ccccc54)c3)c3oc4ccccc4c3c2)cc1 ; CC1(C)c2ccccc2-c2ccc(-c3ccc(N(c4ccccc4)c4cc(-c5cccc(-n6c7ccccc7c7ccccc76)c5)c5oc6ccccc6c5c4)cc3)cc21 ; c1ccc(N(c2ccc(-c3cccc4c3oc3ccccc34)cc2)c2cc(-c3cccc(-n4c5ccccc5c5ccccc54)c3)c3oc4ccccc4c3c2)cc1 ; c1ccc(N(c2ccc(-c3cccc4c3oc3ccccc34)cc2)c2cc(-c3cccc(-n4c5ccccc5c5ccccc54)c3)c3sc4ccccc4c3c2)cc1]

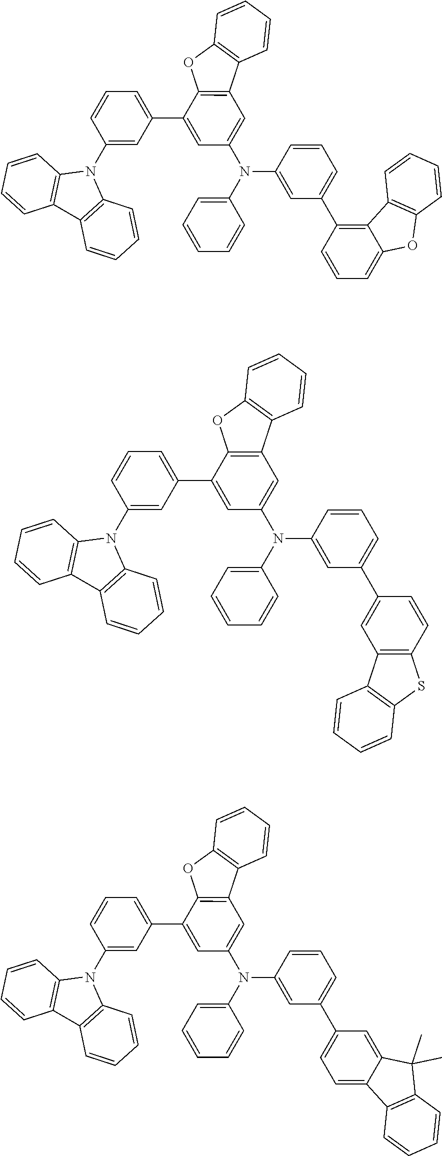 [SMILES: c1ccc(N(c2cccc(-c3cccc4oc5ccccc5c34)c2)c2cc(-c3cccc(-n4c5ccccc5c5ccccc54)c3)c3oc4ccccc4c3c2)cc1 ; c1ccc(N(c2cccc(-c3ccc4sc5ccccc5c4c3)c2)c2cc(-c3cccc(-n4c5ccccc5c5ccccc54)c3)c3oc4ccccc4c3c2)cc1 ; CC1(C)c2ccccc2-c2ccc(-c3cccc(N(c4ccccc4)c4cc(-c5cccc(-n6c7ccccc7c7ccccc76)c5)c5oc6ccccc6c5c4)c3)cc21]

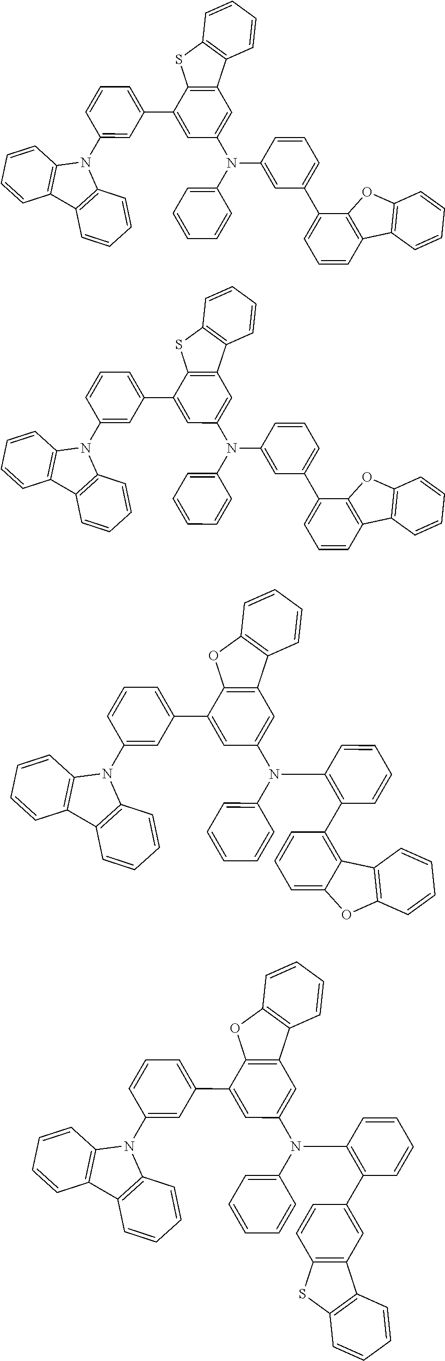 [SMILES: c1ccc(N(c2cccc(-c3cccc4c3oc3ccccc34)c2)c2cc(-c3cccc(-n4c5ccccc5c5ccccc54)c3)c3sc4ccccc4c3c2)cc1 ; c1ccc(N(c2cccc(-c3cccc4c3oc3ccccc34)c2)c2cc(-c3cccc(-n4c5ccccc5c5ccccc54)c3)c3sc4ccccc4c3c2)cc1 ; c1ccc(N(c2cc(-c3cccc(-n4c5ccccc5c5ccccc54)c3)c3oc4ccccc4c3c2)c2ccccc2-c2cccc3oc4ccccc4c23)cc1 ; c1ccc(N(c2cc(-c3cccc(-n4c5ccccc5c5ccccc54)c3)c3oc4ccccc4c3c2)c2ccccc2-c2ccc3sc4ccccc4c3c2)cc1]

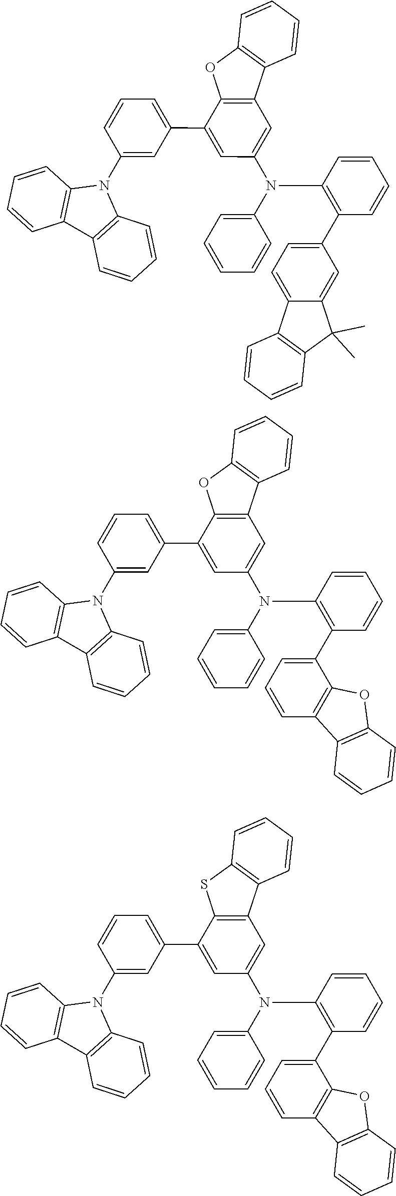

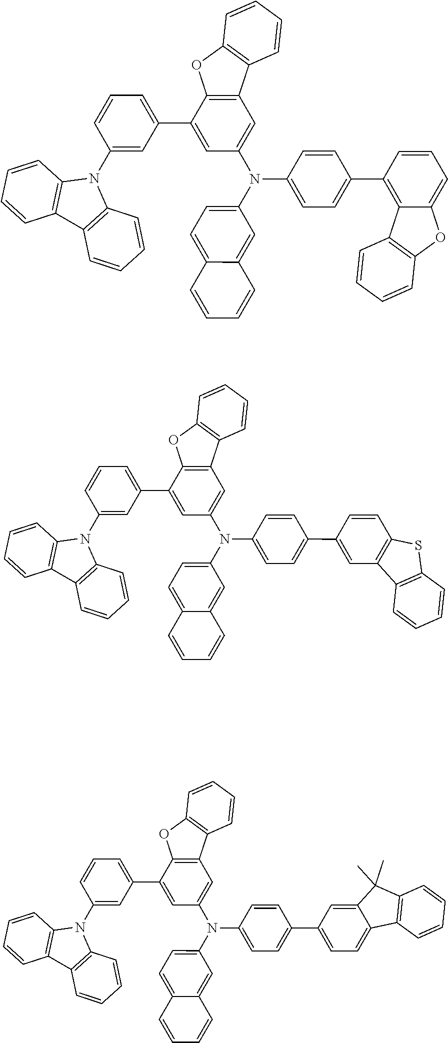 [SMILES: c1cc(-c2cc(N(c3ccc(-c4cccc5oc6ccccc6c45)cc3)c3ccc4ccccc4c3)cc3c2oc2ccccc23)cc(-n2c3ccccc3c3ccccc32)c1 ; c1cc(-c2cc(N(c3ccc(-c4ccc5sc6ccccc6c5c4)cc3)c3ccc4ccccc4c3)cc3c2oc2ccccc23)cc(-n2c3ccccc3c3ccccc32)c1 ; CC1(C)c2ccccc2-c2ccc(-c3ccc(N(c4ccc5ccccc5c4)c4cc(-c5cccc(-n6c7ccccc7c7ccccc76)c5)c5oc6ccccc6c5c4)cc3)cc21]

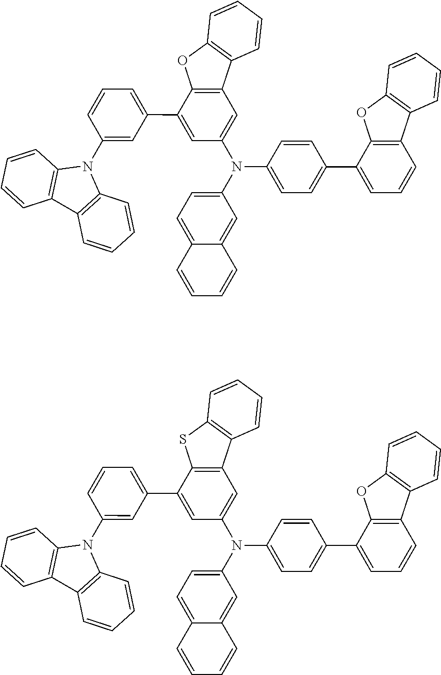 [SMILES: c1cc(-c2cc(N(c3ccc(-c4cccc5c4oc4ccccc45)cc3)c3ccc4ccccc4c3)cc3c2oc2ccccc23)cc(-n2c3ccccc3c3ccccc32)c1 ; c1cc(-c2cc(N(c3ccc(-c4cccc5c4oc4ccccc45)cc3)c3ccc4ccccc4c3)cc3c2sc2ccccc23)cc(-n2c3ccccc3c3ccccc32)c1]

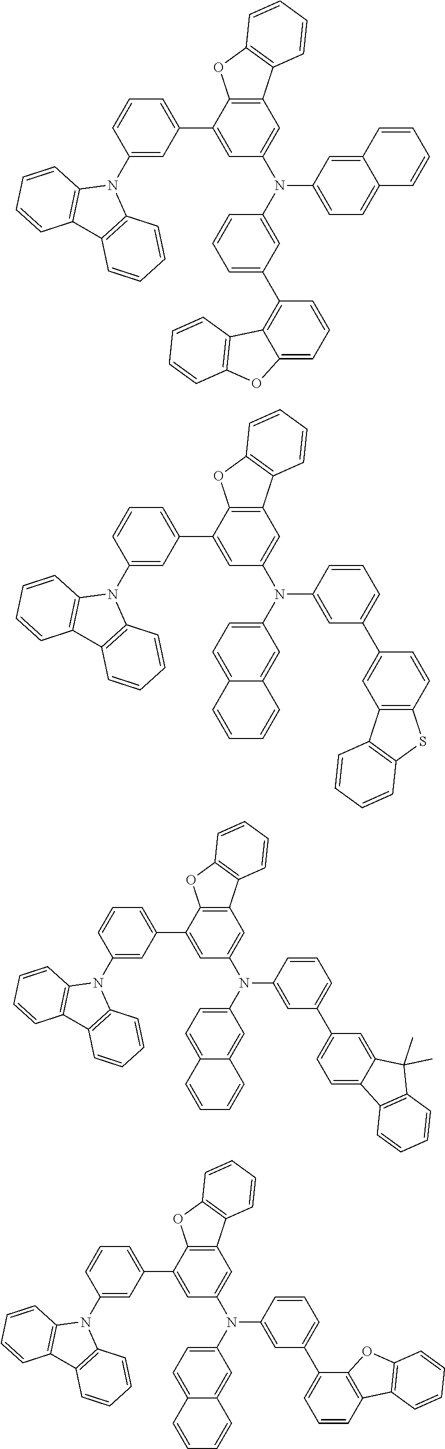

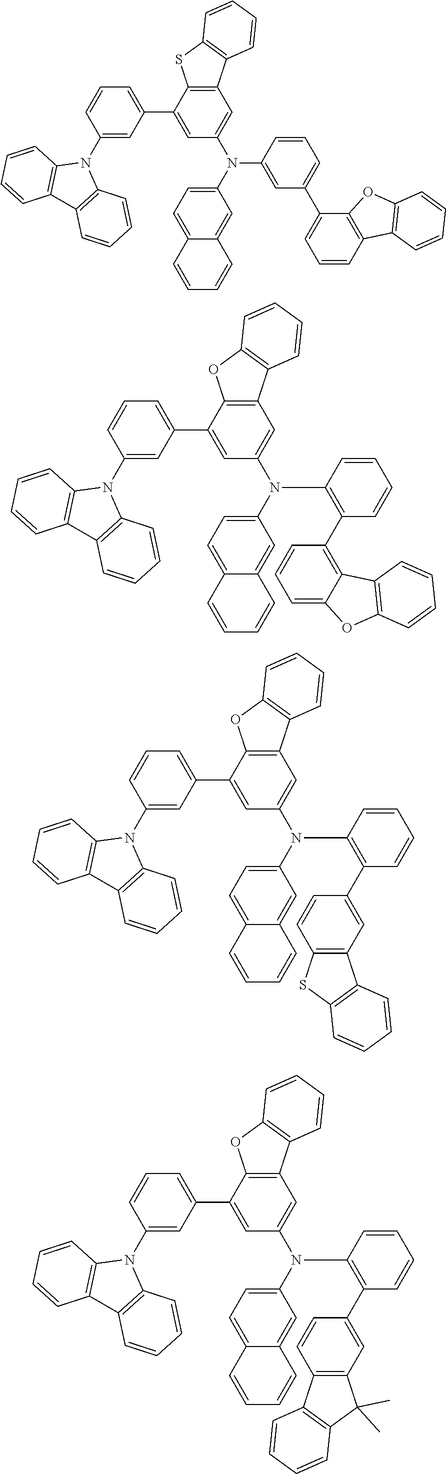

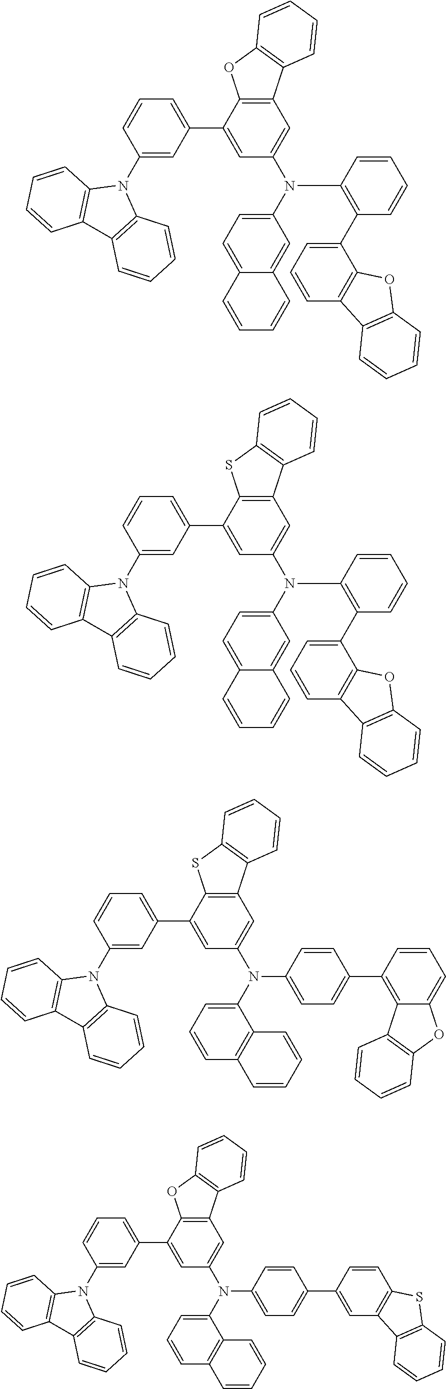 [SMILES: c1cc(-c2cc(N(c3ccc4ccccc4c3)c3ccccc3-c3cccc4c3oc3ccccc34)cc3c2oc2ccccc23)cc(-n2c3ccccc3c3ccccc32)c1 ; c1cc(-c2cc(N(c3ccc4ccccc4c3)c3ccccc3-c3cccc4c3oc3ccccc34)cc3c2sc2ccccc23)cc(-n2c3ccccc3c3ccccc32)c1 ; c1cc(-c2cc(N(c3ccc(-c4cccc5oc6ccccc6c45)cc3)c3cccc4ccccc34)cc3c2sc2ccccc23)cc(-n2c3ccccc3c3ccccc32)c1 ; c1cc(-c2cc(N(c3ccc(-c4ccc5sc6ccccc6c5c4)cc3)c3cccc4ccccc34)cc3c2oc2ccccc23)cc(-n2c3ccccc3c3ccccc32)c1]

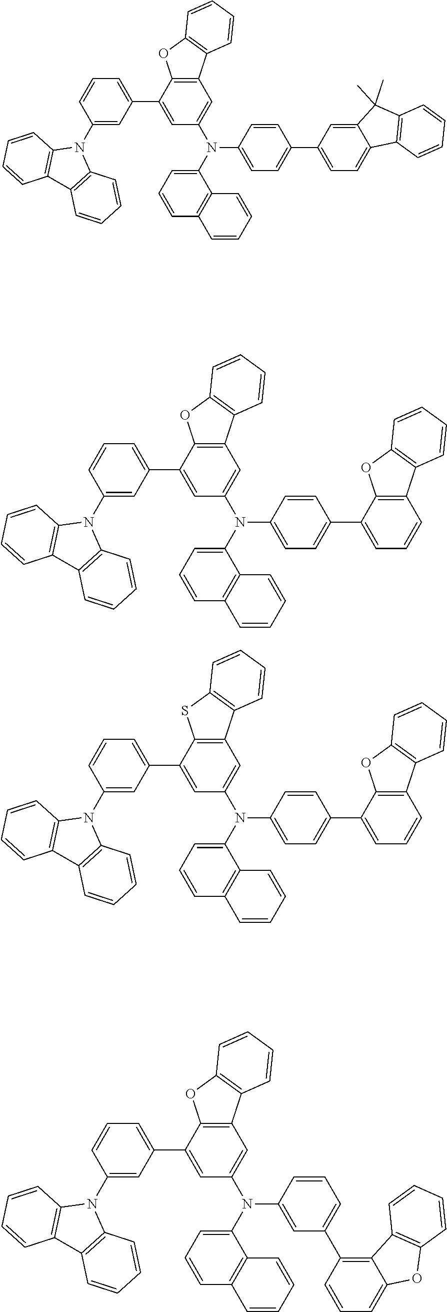

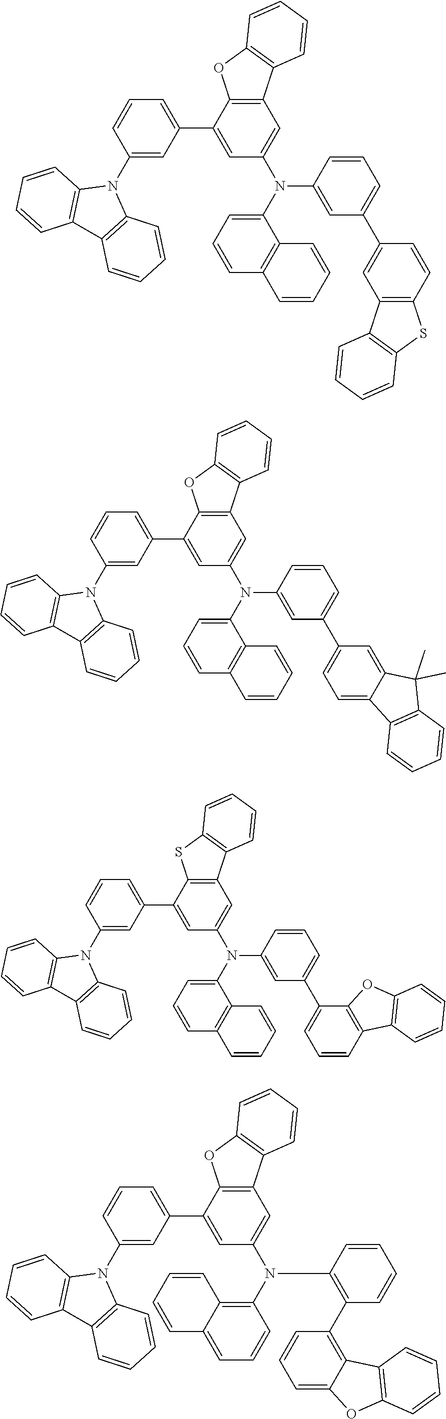

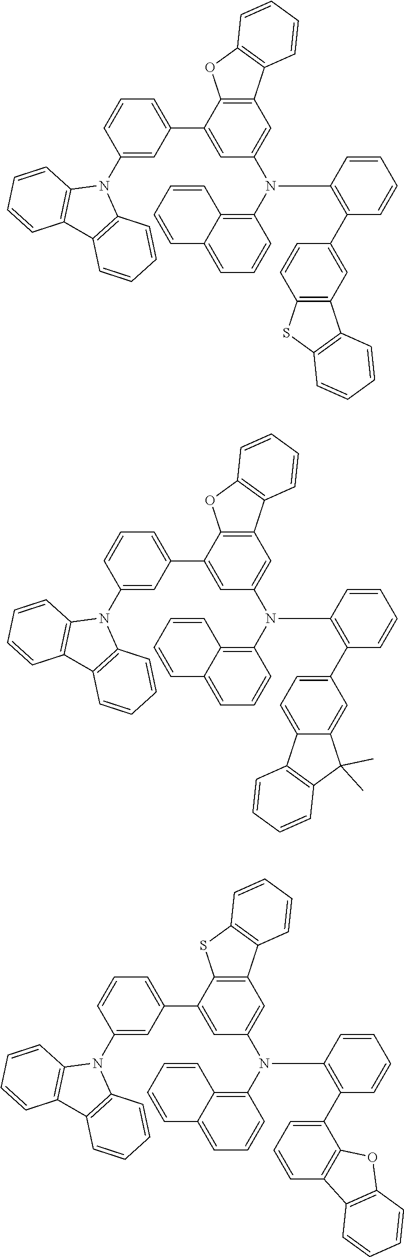 [SMILES: c1cc(-c2cc(N(c3ccccc3-c3ccc4sc5ccccc5c4c3)c3cccc4ccccc34)cc3c2oc2ccccc23)cc(-n2c3ccccc3c3ccccc32)c1 ; CC1(C)c2ccccc2-c2ccc(-c3ccccc3N(c3cc(-c4cccc(-n5c6ccccc6c6ccccc65)c4)c4oc5ccccc5c4c3)c3cccc4ccccc34)cc21 ; c1cc(-c2cc(N(c3ccccc3-c3cccc4c3oc3ccccc34)c3cccc4ccccc34)cc3c2sc2ccccc23)cc(-n2c3ccccc3c3ccccc32)c1]

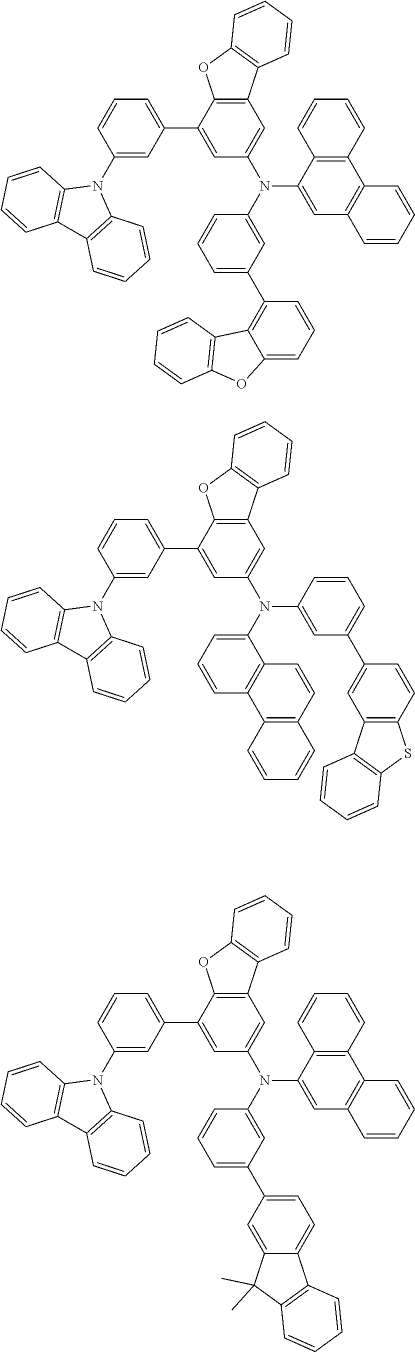

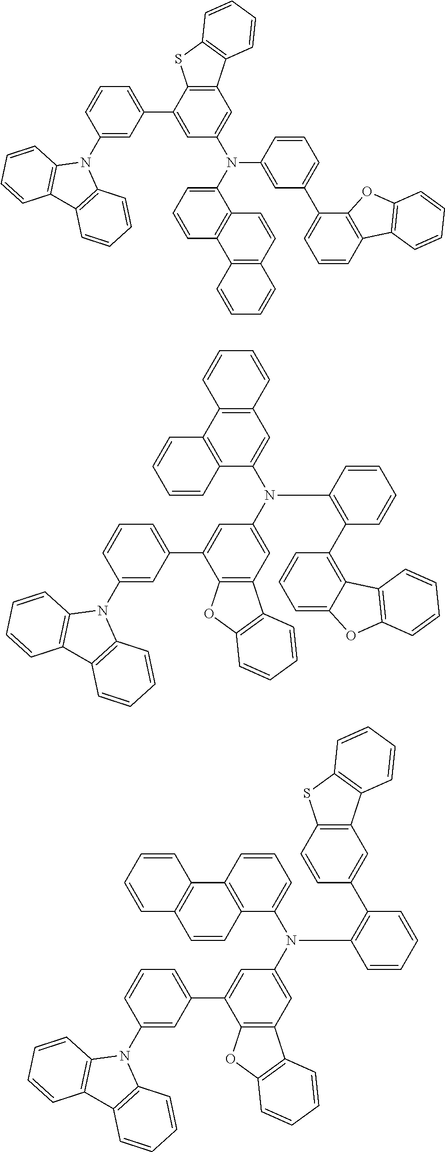

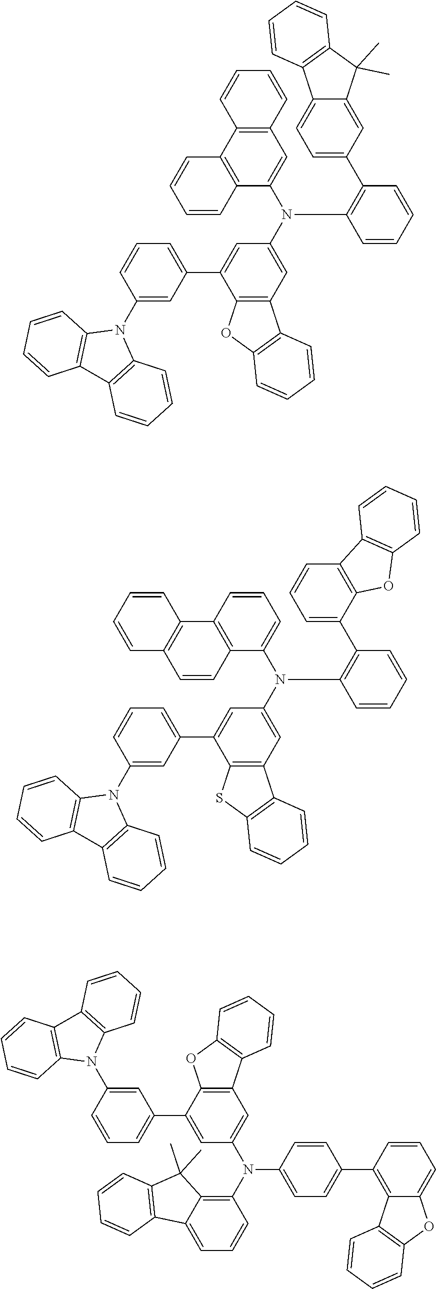 [SMILES: CC1(C)c2ccccc2-c2ccc(-c3ccccc3N(c3cc(-c4cccc(-n5c6ccccc6c6ccccc65)c4)c4oc5ccccc5c4c3)c3cc4ccccc4c4ccccc34)cc21 ; c1cc(-c2cc(N(c3ccccc3-c3cccc4c3oc3ccccc34)c3cccc4c3ccc3ccccc34)cc3c2sc2ccccc23)cc(-n2c3ccccc3c3ccccc32)c1 ; CC1(C)c2ccccc2-c2cccc(N(c3ccc(-c4cccc5oc6ccccc6c45)cc3)c3cc(-c4cccc(-n5c6ccccc6c6ccccc65)c4)c4oc5ccccc5c4c3)c21]

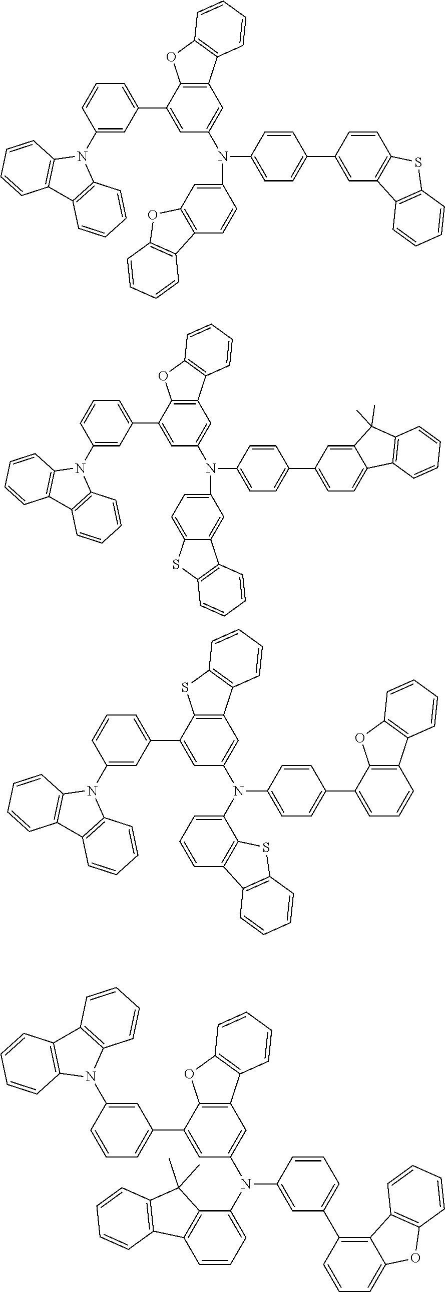

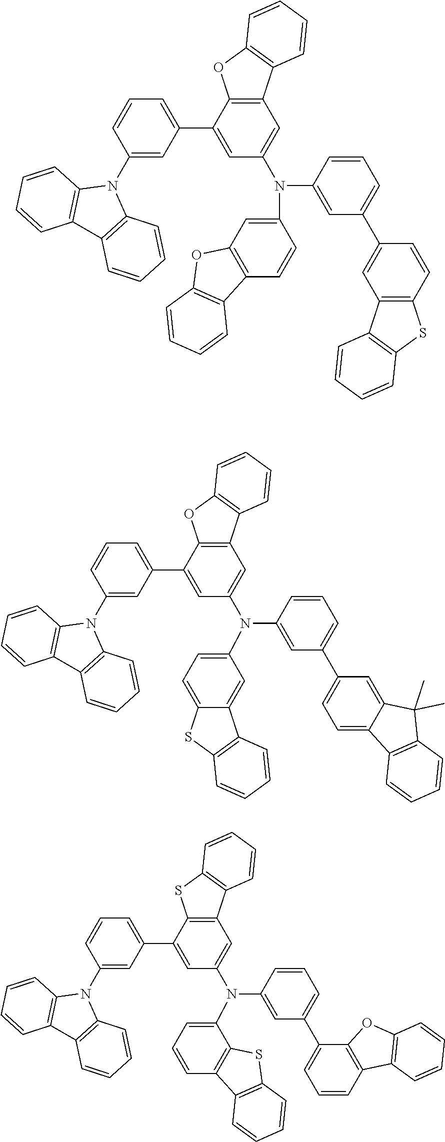

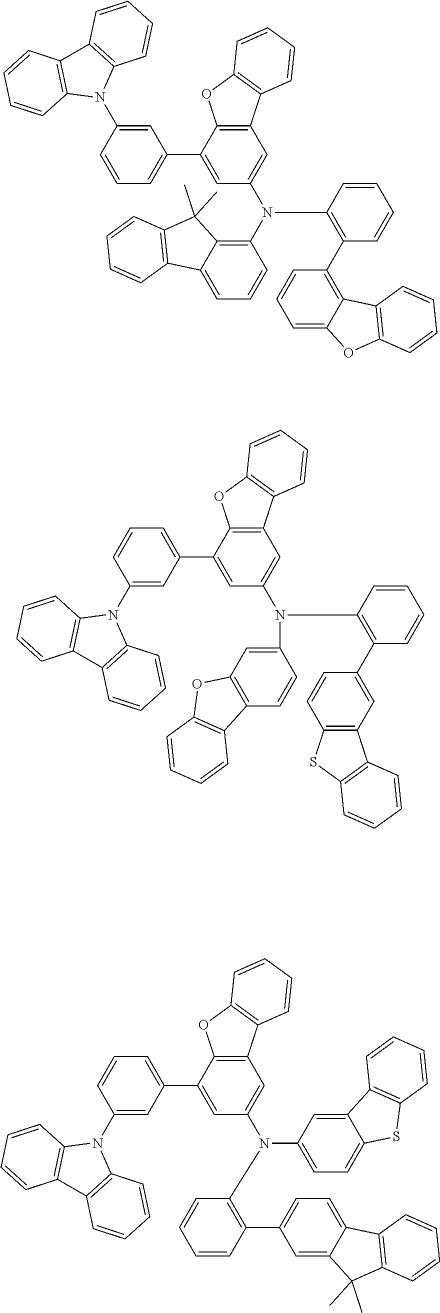 [SMILES: CC1(C)c2ccccc2-c2cccc(N(c3cc(-c4cccc(-n5c6ccccc6c6ccccc65)c4)c4oc5ccccc5c4c3)c3ccccc3-c3cccc4oc5ccccc5c34)c21 ; c1cc(-c2cc(N(c3ccc4c(c3)oc3ccccc34)c3ccccc3-c3ccc4sc5ccccc5c4c3)cc3c2oc2ccccc23)cc(-n2c3ccccc3c3ccccc32)c1 ; CC1(C)c2ccccc2-c2ccc(-c3ccccc3N(c3ccc4sc5ccccc5c4c3)c3cc(-c4cccc(-n5c6ccccc6c6ccccc65)c4)c4oc5ccccc5c4c3)cc21]

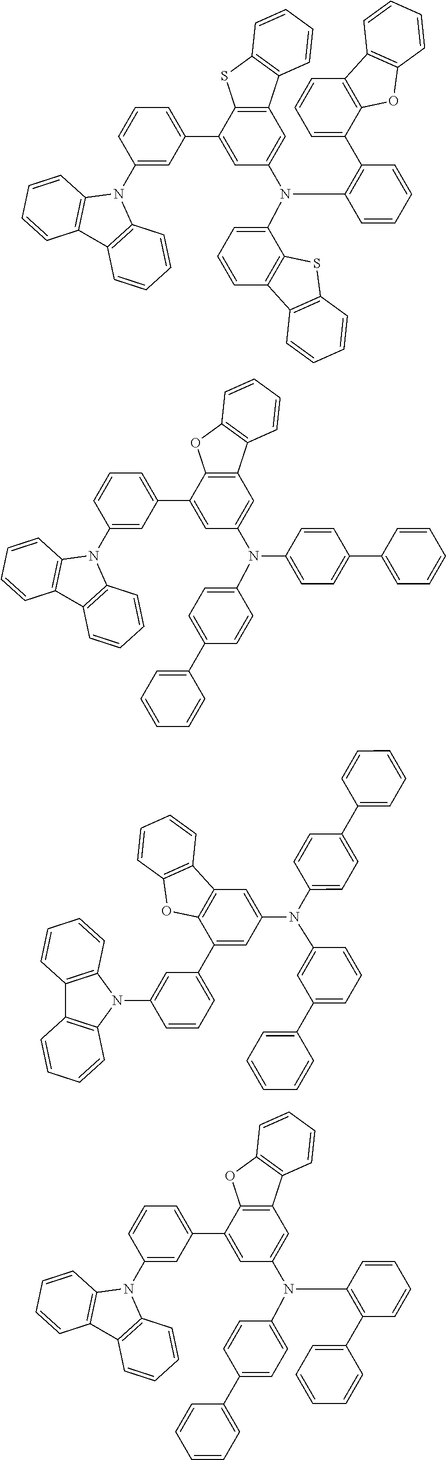

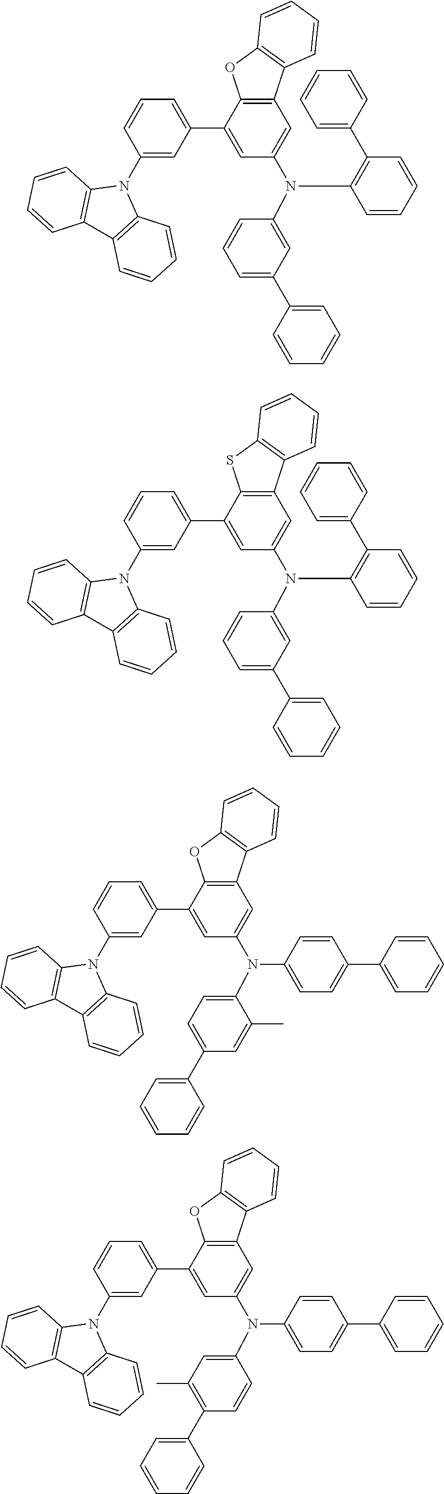

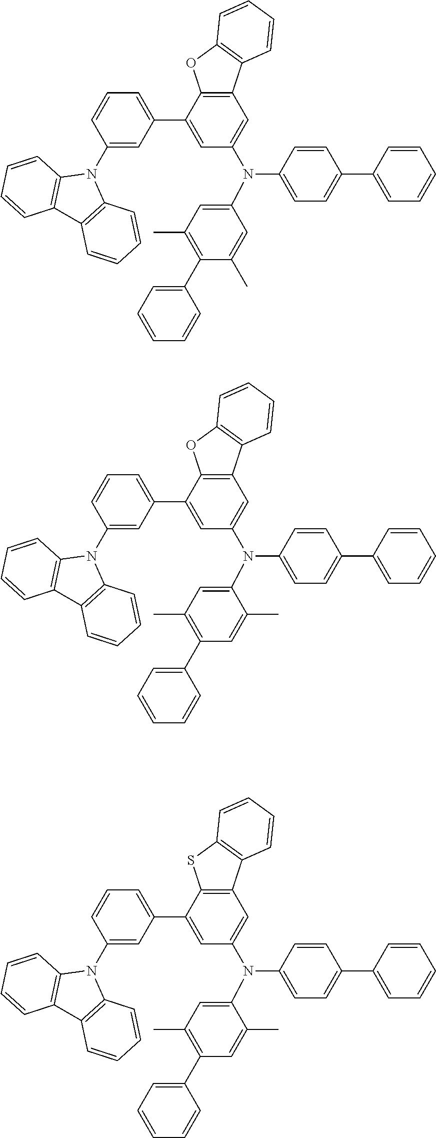 [SMILES: Cc1cc(N(c2ccc(-c3ccccc3)cc2)c2cc(-c3cccc(-n4c5ccccc5c5ccccc54)c3)c3oc4ccccc4c3c2)cc(C)c1-c1ccccc1 ; Cc1cc(N(c2ccc(-c3ccccc3)cc2)c2cc(-c3cccc(-n4c5ccccc5c5ccccc54)c3)c3oc4ccccc4c3c2)c(C)cc1-c1ccccc1 ; Cc1cc(N(c2ccc(-c3ccccc3)cc2)c2cc(-c3cccc(-n4c5ccccc5c5ccccc54)c3)c3sc4ccccc4c3c2)c(C)cc1-c1ccccc1]

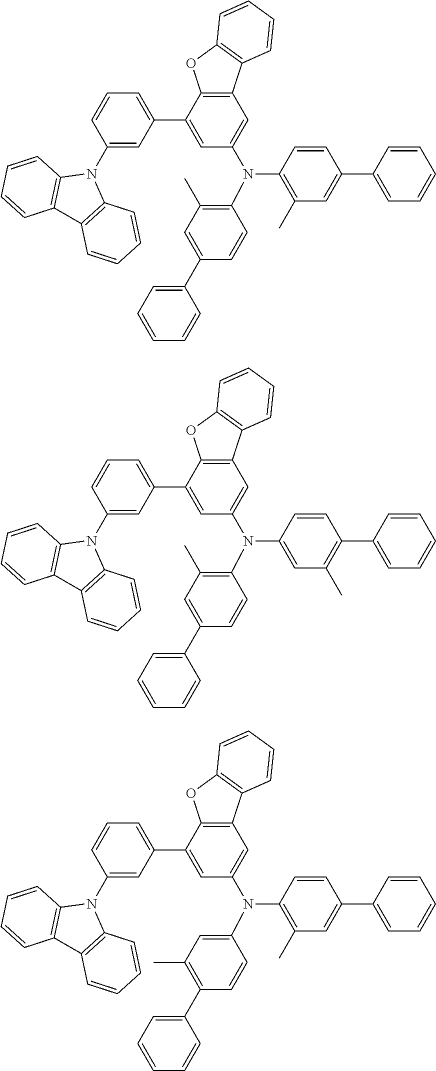 [SMILES: Cc1cc(-c2ccccc2)ccc1N(c1cc(-c2cccc(-n3c4ccccc4c4ccccc43)c2)c2oc3ccccc3c2c1)c1ccc(-c2ccccc2)cc1C ; Cc1cc(N(c2cc(-c3cccc(-n4c5ccccc5c5ccccc54)c3)c3oc4ccccc4c3c2)c2ccc(-c3ccccc3)cc2C)ccc1-c1ccccc1 ; Cc1cc(N(c2cc(-c3cccc(-n4c5ccccc5c5ccccc54)c3)c3oc4ccccc4c3c2)c2ccc(-c3ccccc3)cc2C)ccc1-c1ccccc1]

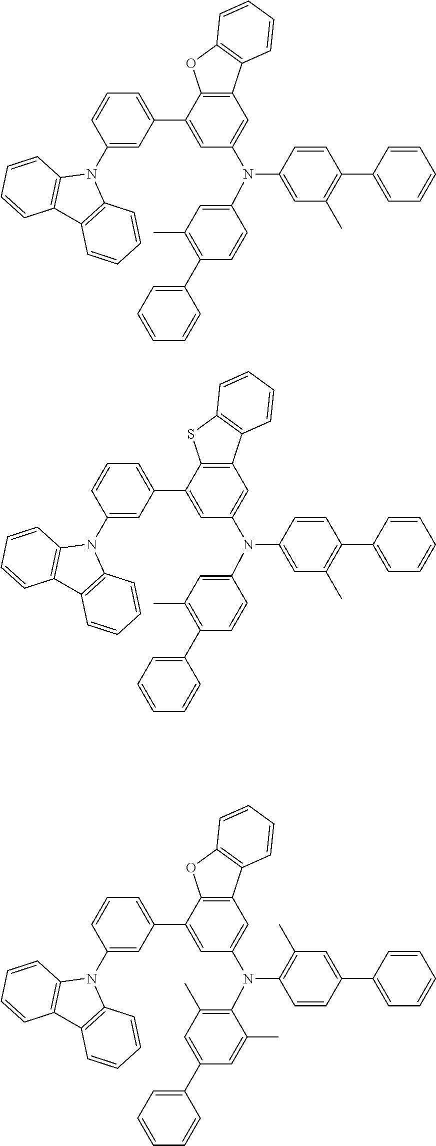 [SMILES: Cc1cc(N(c2ccc(-c3ccccc3)c(C)c2)c2cc(-c3cccc(-n4c5ccccc5c5ccccc54)c3)c3oc4ccccc4c3c2)ccc1-c1ccccc1 ; Cc1cc(N(c2ccc(-c3ccccc3)c(C)c2)c2cc(-c3cccc(-n4c5ccccc5c5ccccc54)c3)c3sc4ccccc4c3c2)ccc1-c1ccccc1 ; Cc1cc(-c2ccccc2)ccc1N(c1cc(-c2cccc(-n3c4ccccc4c4ccccc43)c2)c2oc3ccccc3c2c1)c1c(C)cc(-c2ccccc2)cc1C]

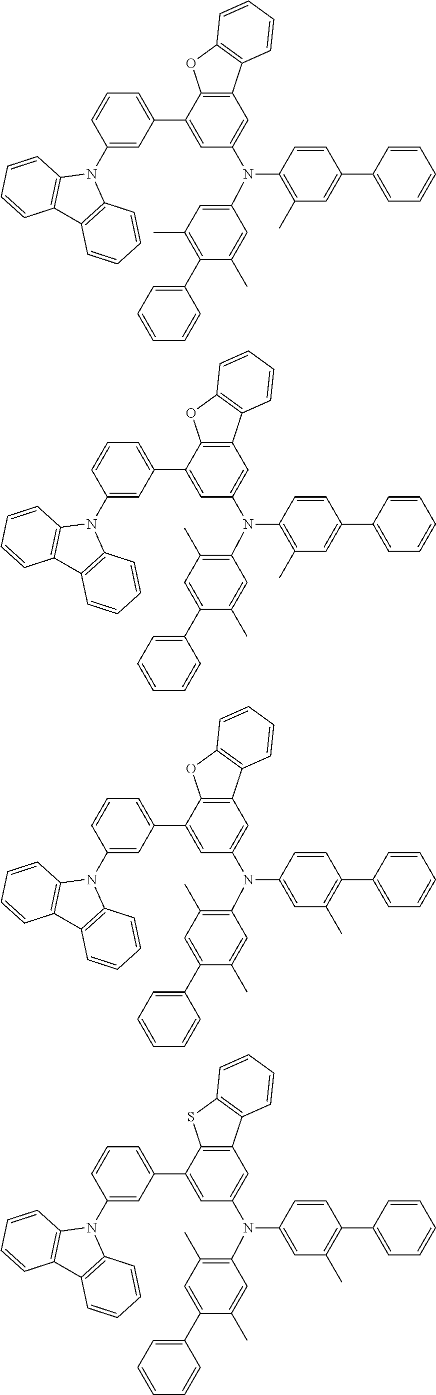

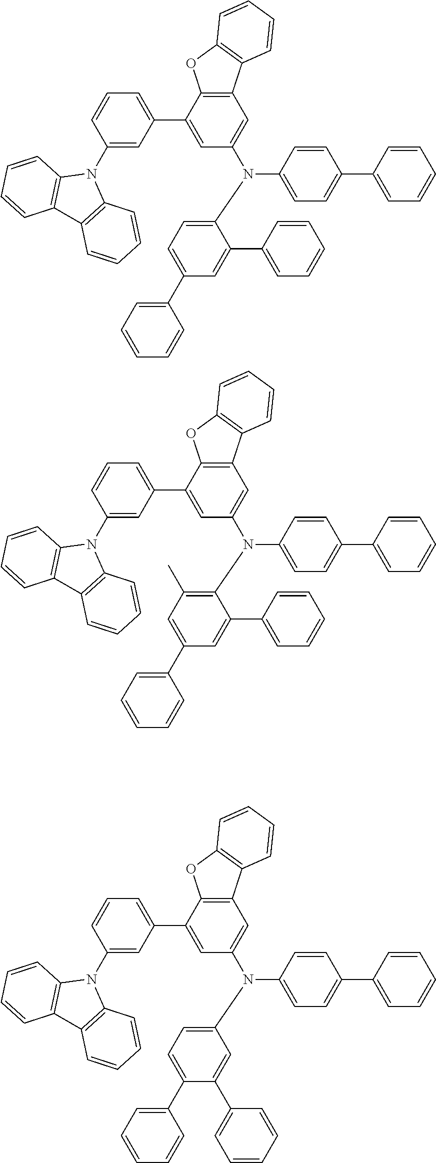 [SMILES: c1ccc(-c2ccc(N(c3cc(-c4cccc(-n5c6ccccc6c6ccccc65)c4)c4oc5ccccc5c4c3)c3ccc(-c4ccccc4)cc3-c3ccccc3)cc2)cc1 ; Cc1cc(-c2ccccc2)cc(-c2ccccc2)c1N(c1ccc(-c2ccccc2)cc1)c1cc(-c2cccc(-n3c4ccccc4c4ccccc43)c2)c2oc3ccccc3c2c1 ; c1ccc(-c2ccc(N(c3ccc(-c4ccccc4)c(-c4ccccc4)c3)c3cc(-c4cccc(-n5c6ccccc6c6ccccc65)c4)c4oc5ccccc5c4c3)cc2)cc1]

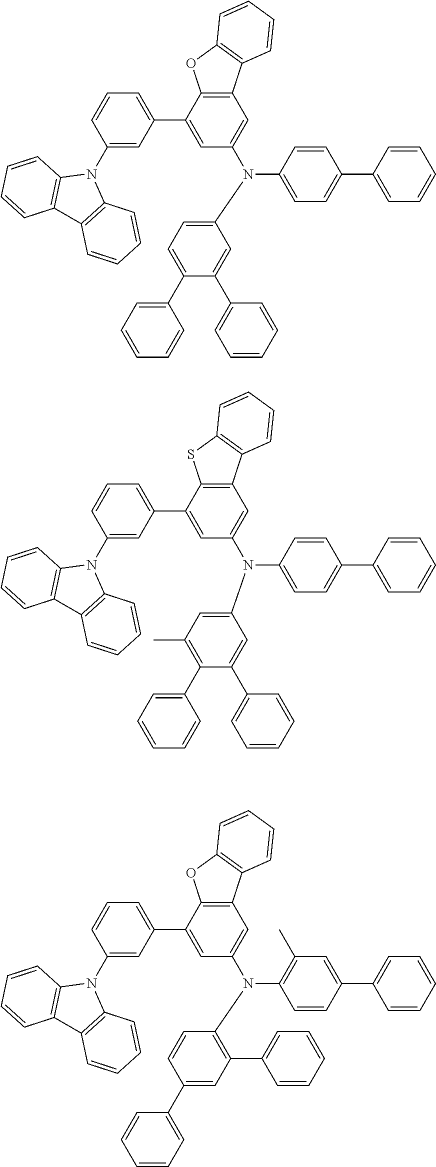

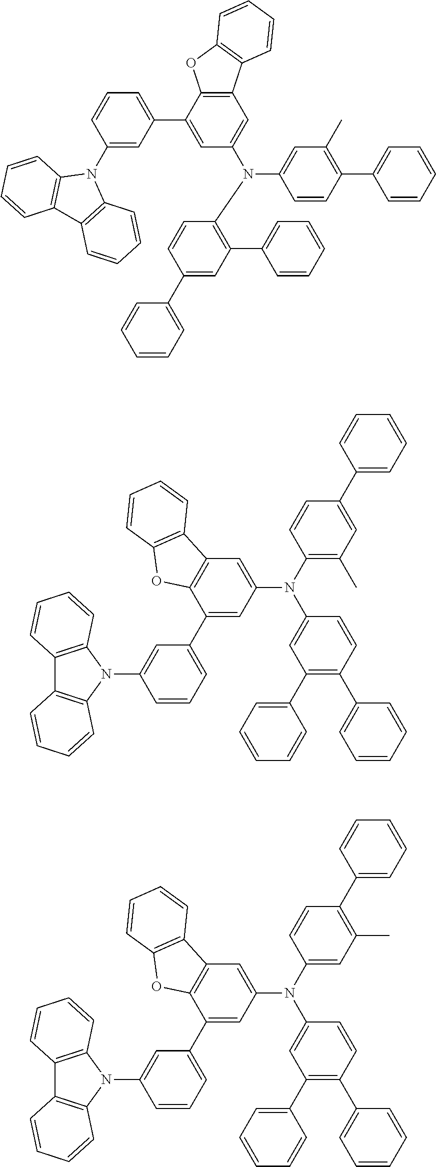 [SMILES: Cc1cc(N(c2cc(-c3cccc(-n4c5ccccc5c5ccccc54)c3)c3oc4ccccc4c3c2)c2ccc(-c3ccccc3)cc2-c2ccccc2)ccc1-c1ccccc1 ; Cc1cc(-c2ccccc2)ccc1N(c1ccc(-c2ccccc2)c(-c2ccccc2)c1)c1cc(-c2cccc(-n3c4ccccc4c4ccccc43)c2)c2oc3ccccc3c2c1 ; Cc1cc(N(c2ccc(-c3ccccc3)c(-c3ccccc3)c2)c2cc(-c3cccc(-n4c5ccccc5c5ccccc54)c3)c3oc4ccccc4c3c2)ccc1-c1ccccc1]

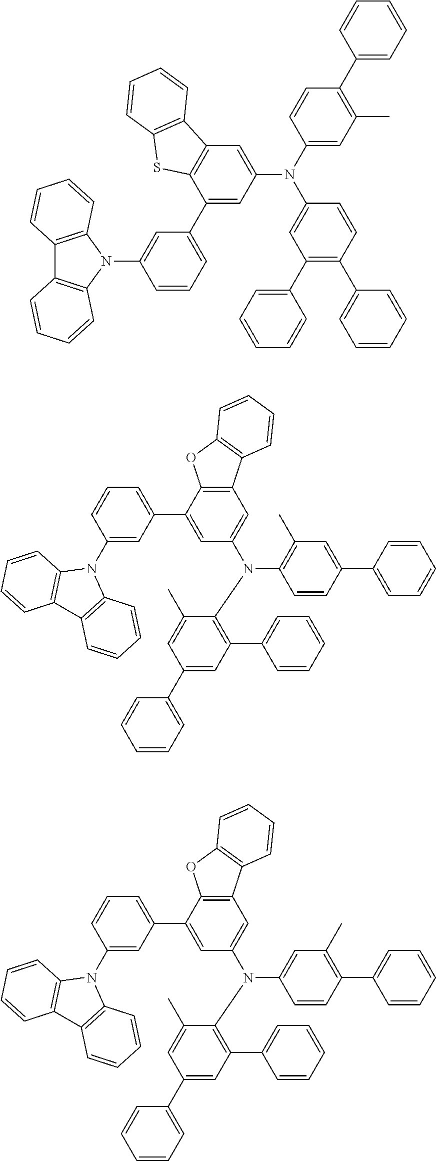 [SMILES: Cc1cc(N(c2ccc(-c3ccccc3)c(-c3ccccc3)c2)c2cc(-c3cccc(-n4c5ccccc5c5ccccc54)c3)c3sc4ccccc4c3c2)ccc1-c1ccccc1 ; Cc1cc(-c2ccccc2)ccc1N(c1cc(-c2cccc(-n3c4ccccc4c4ccccc43)c2)c2oc3ccccc3c2c1)c1c(C)cc(-c2ccccc2)cc1-c1ccccc1 ; Cc1cc(N(c2cc(-c3cccc(-n4c5ccccc5c5ccccc54)c3)c3oc4ccccc4c3c2)c2c(C)cc(-c3ccccc3)cc2-c2ccccc2)ccc1-c1ccccc1]

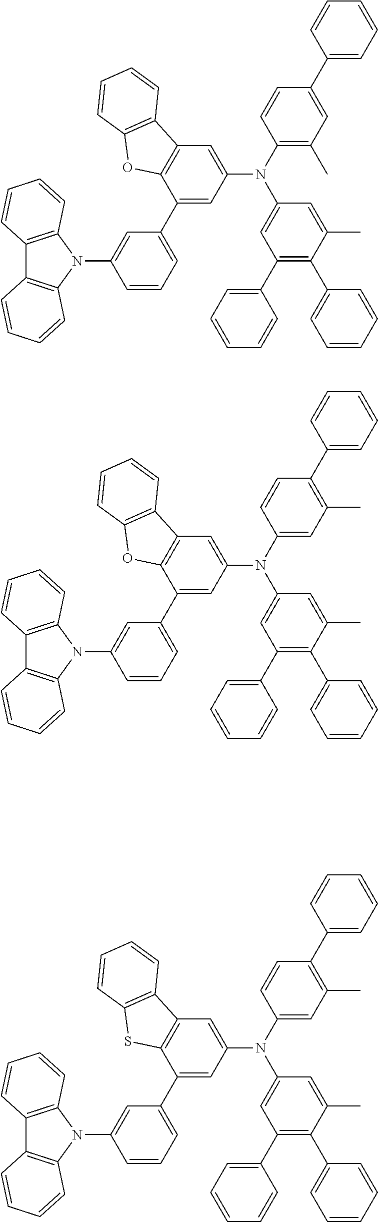

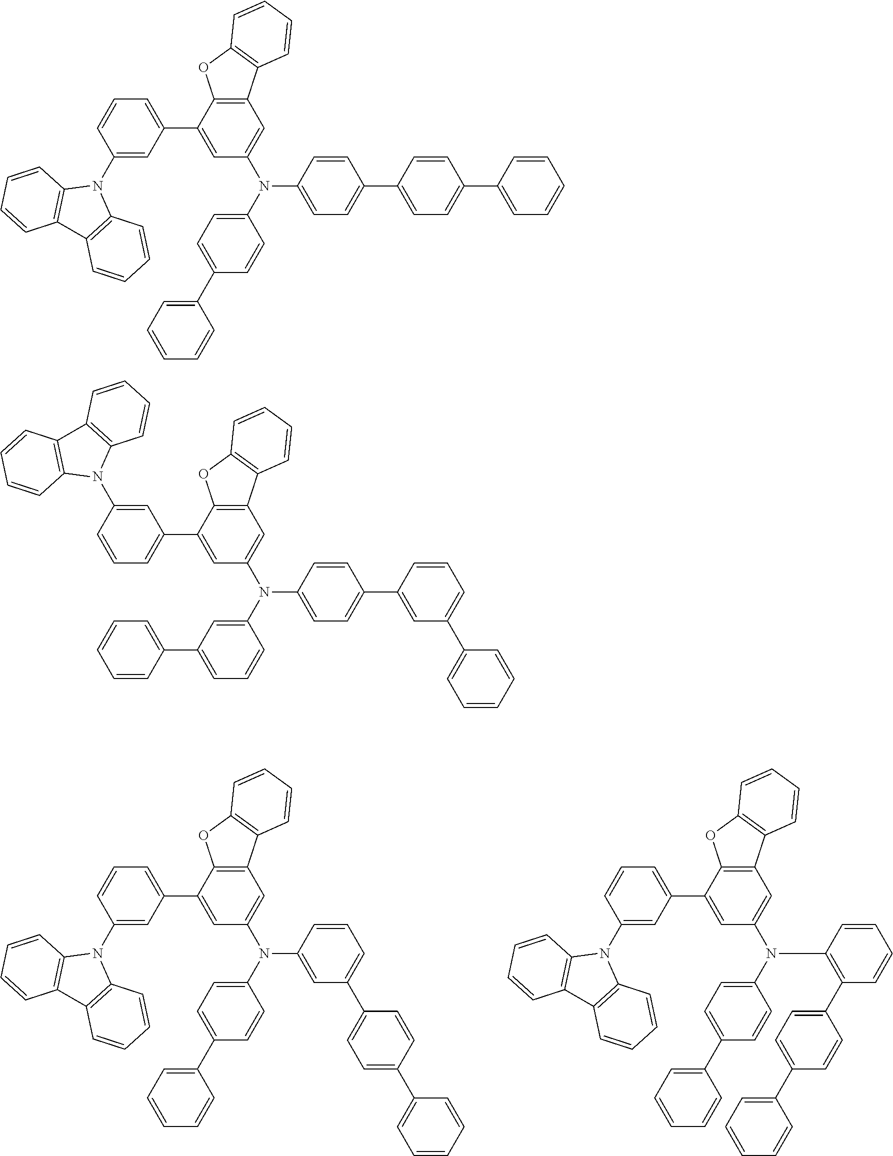

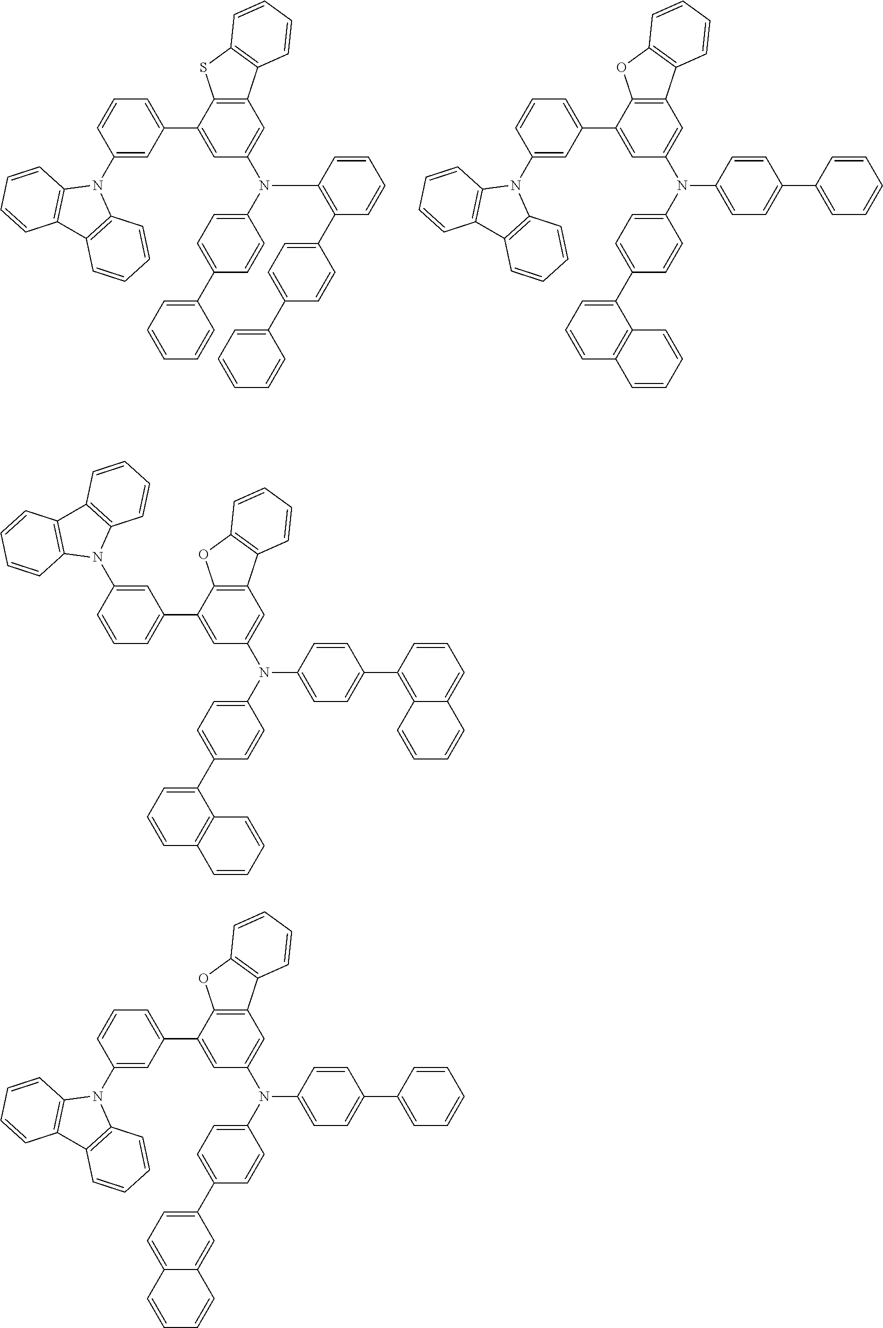

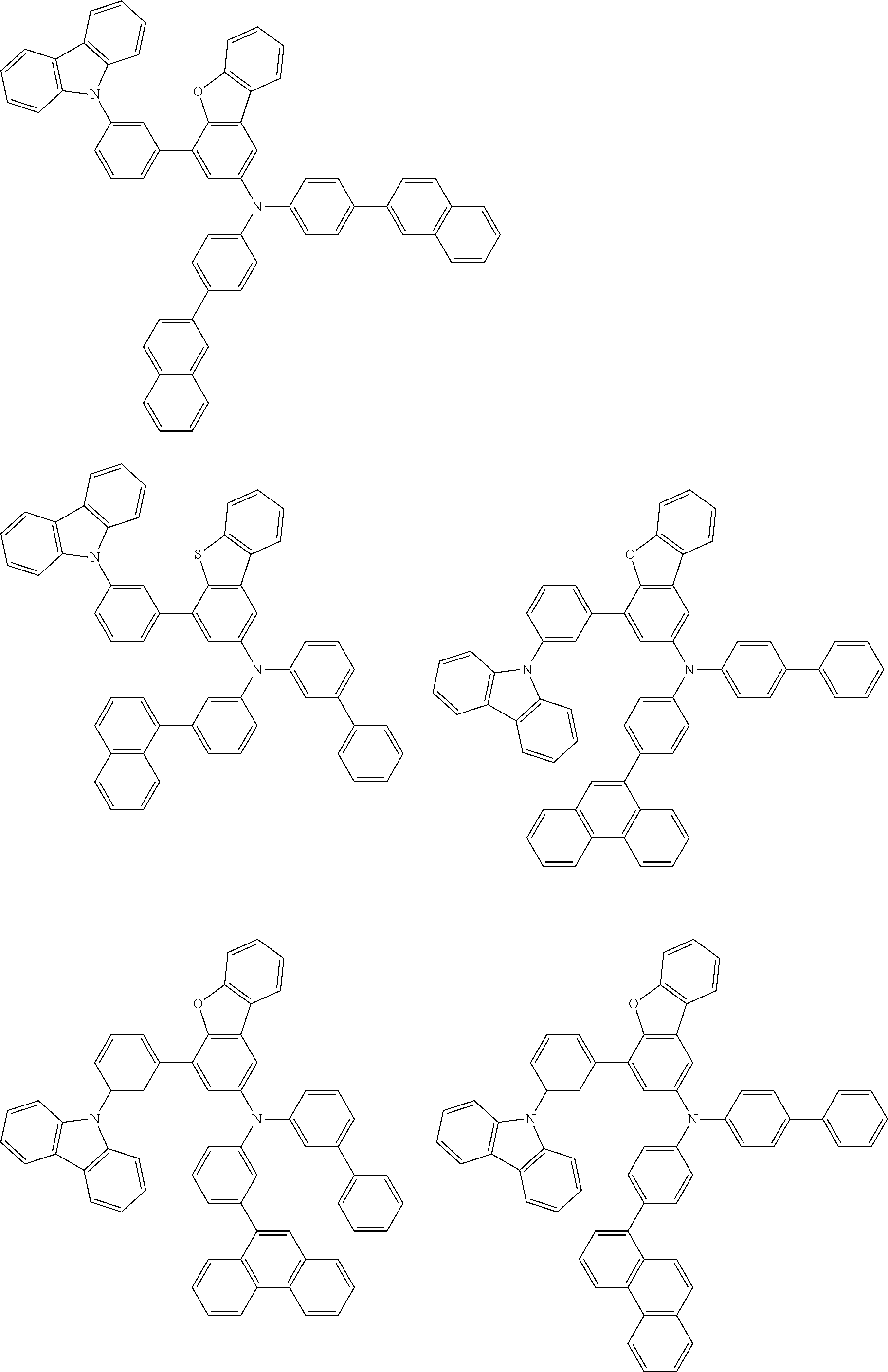

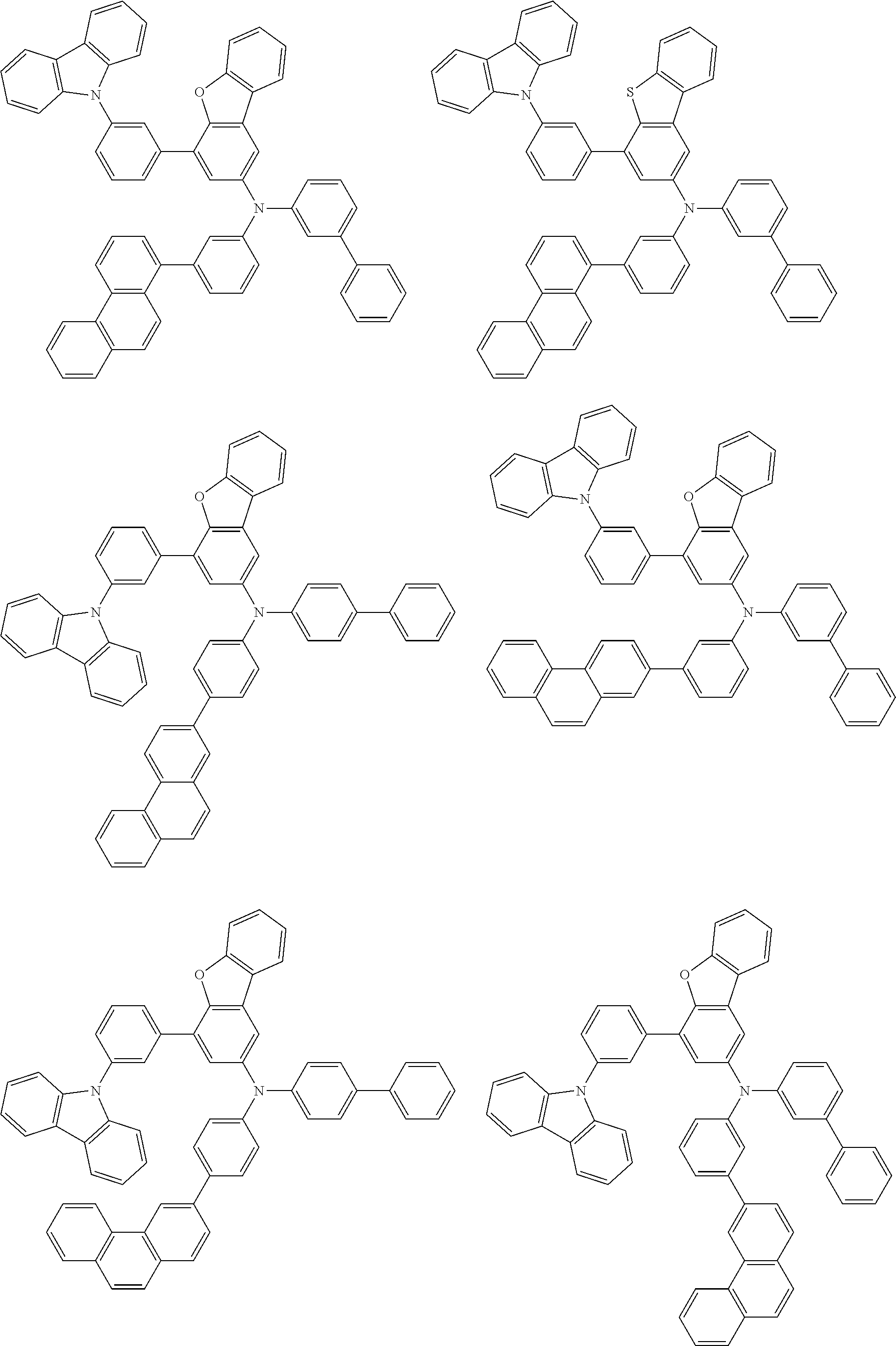

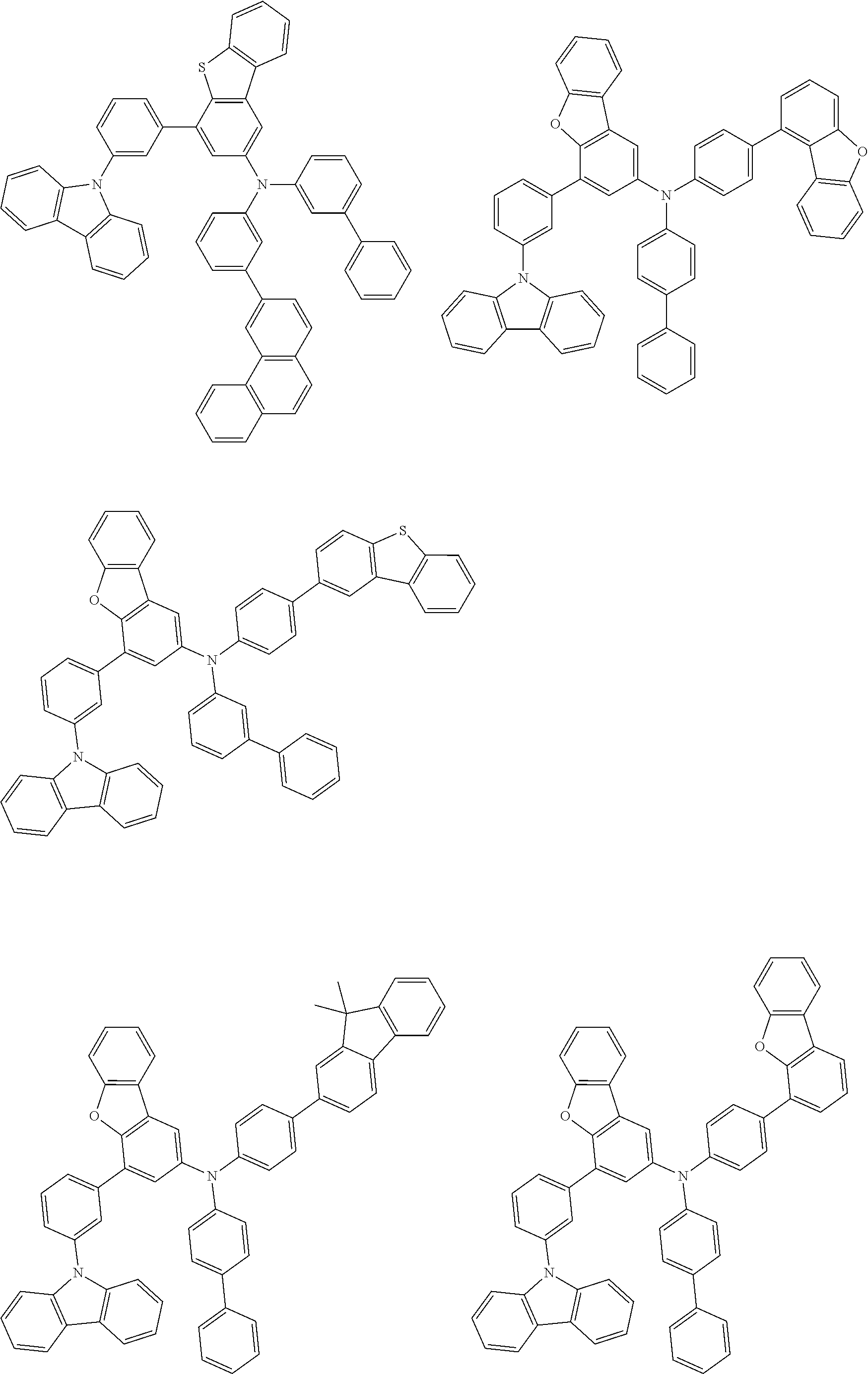

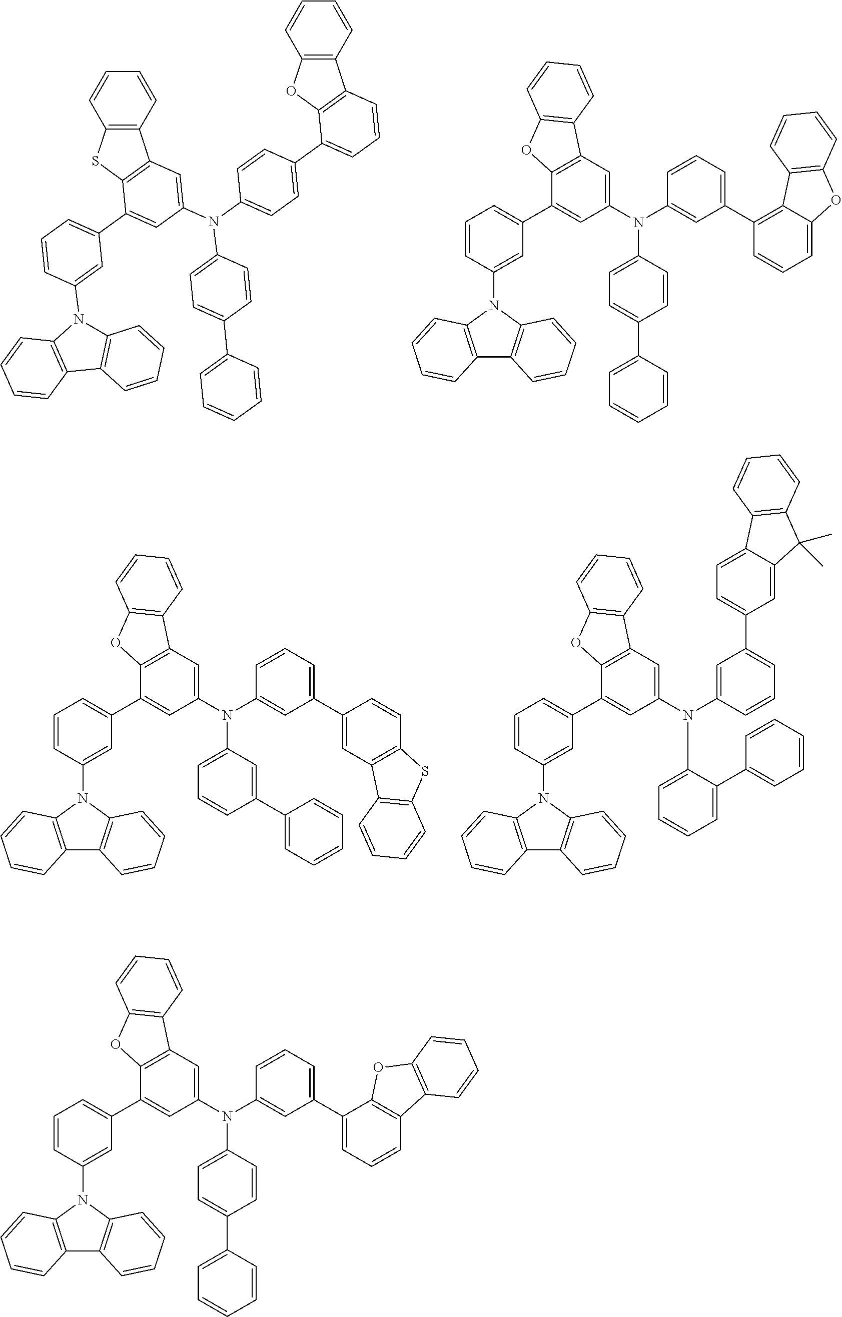

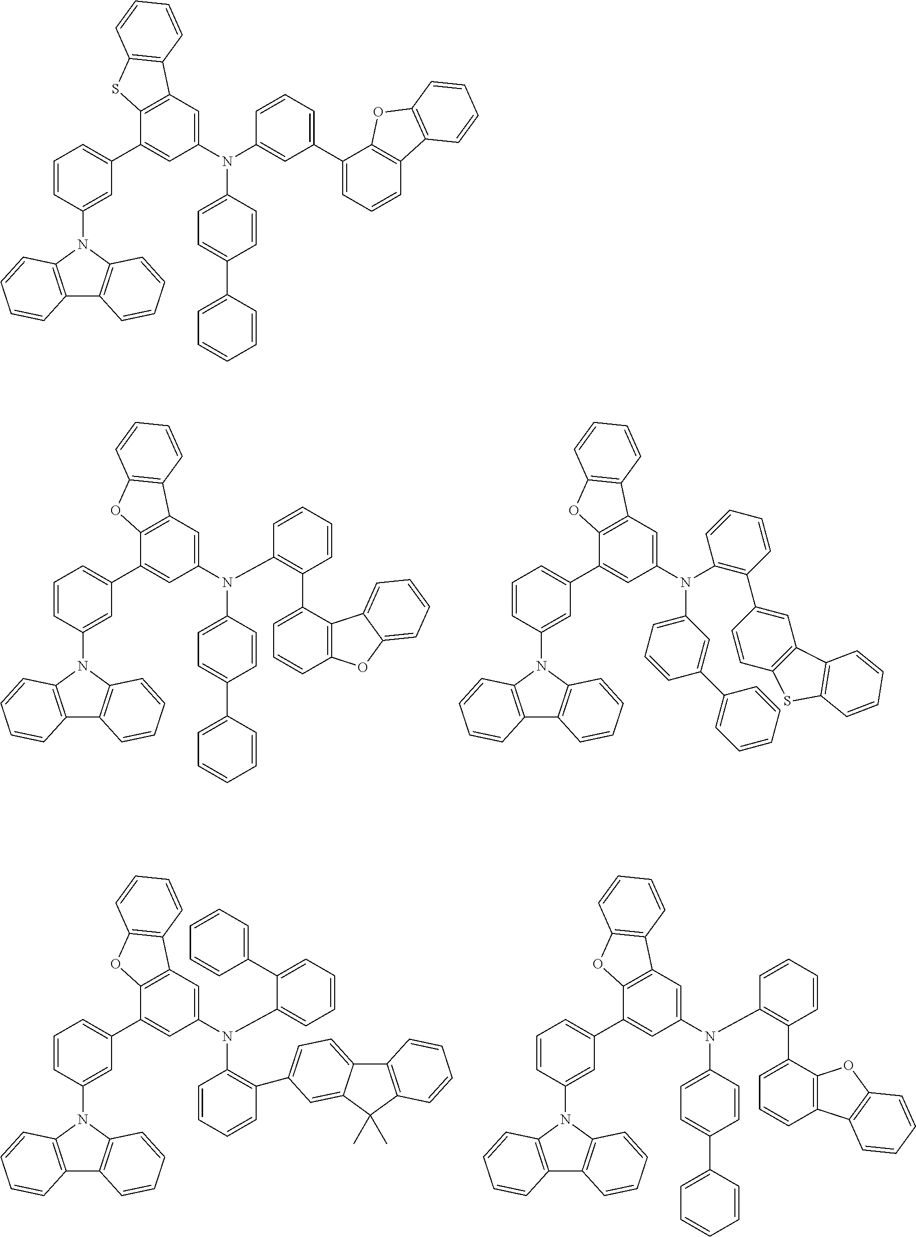

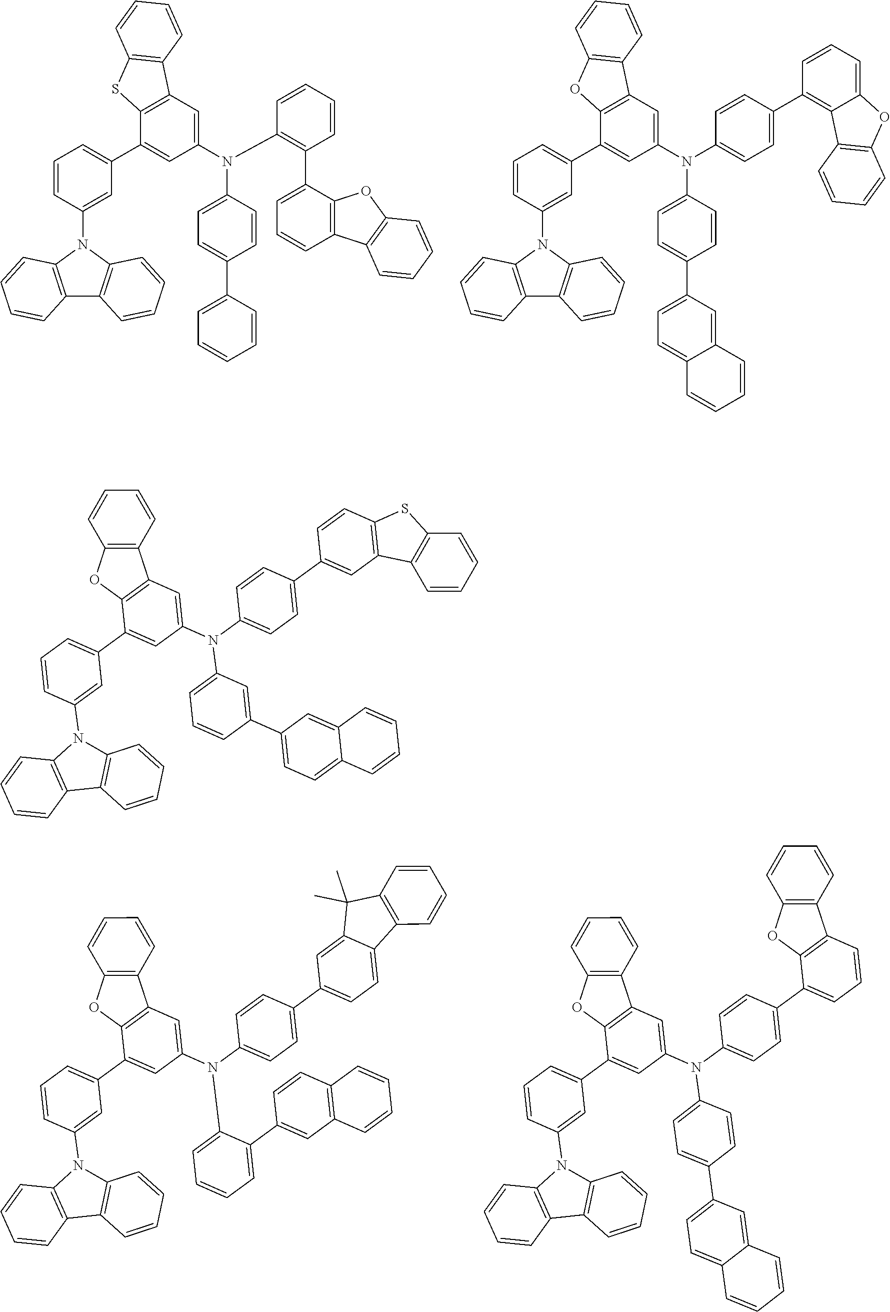

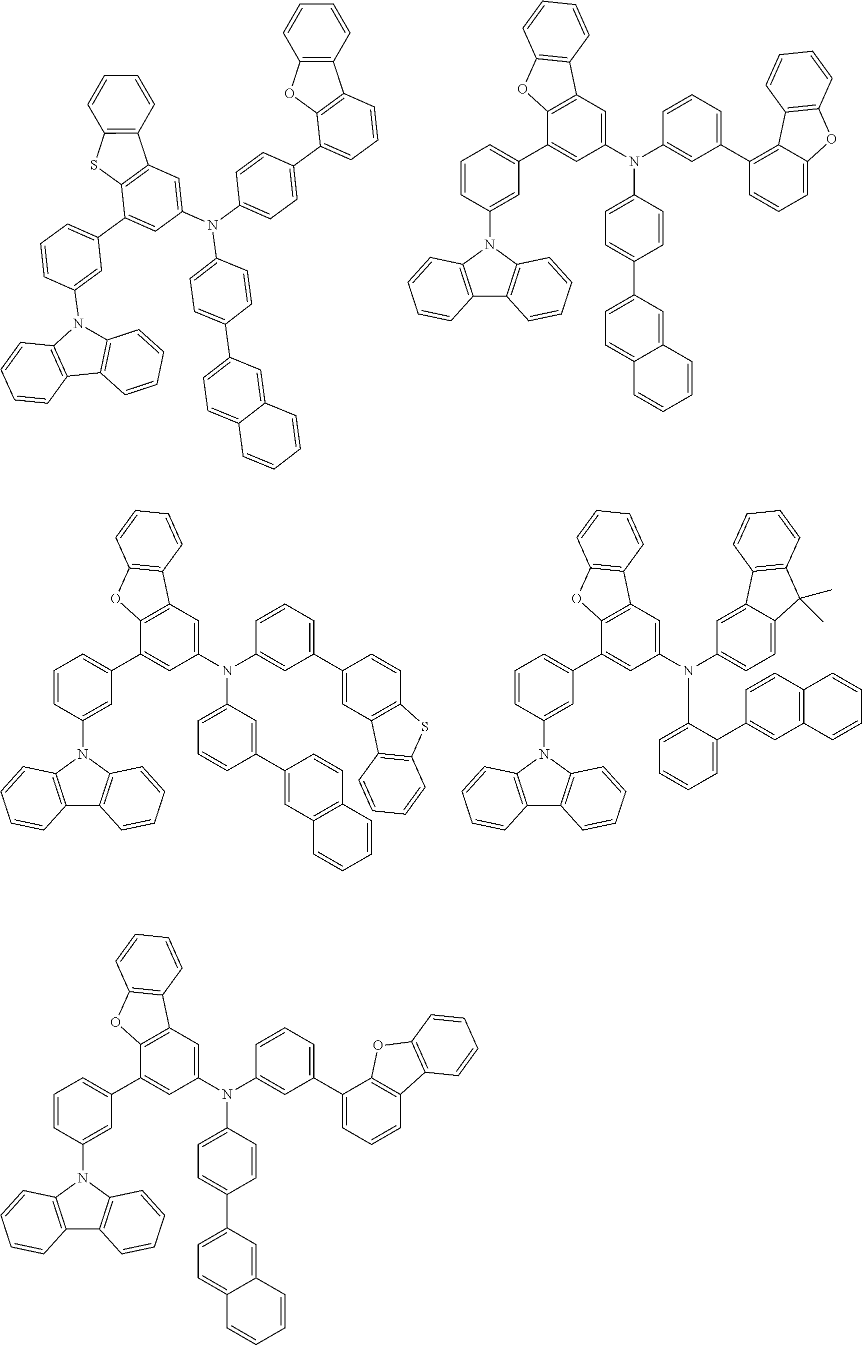

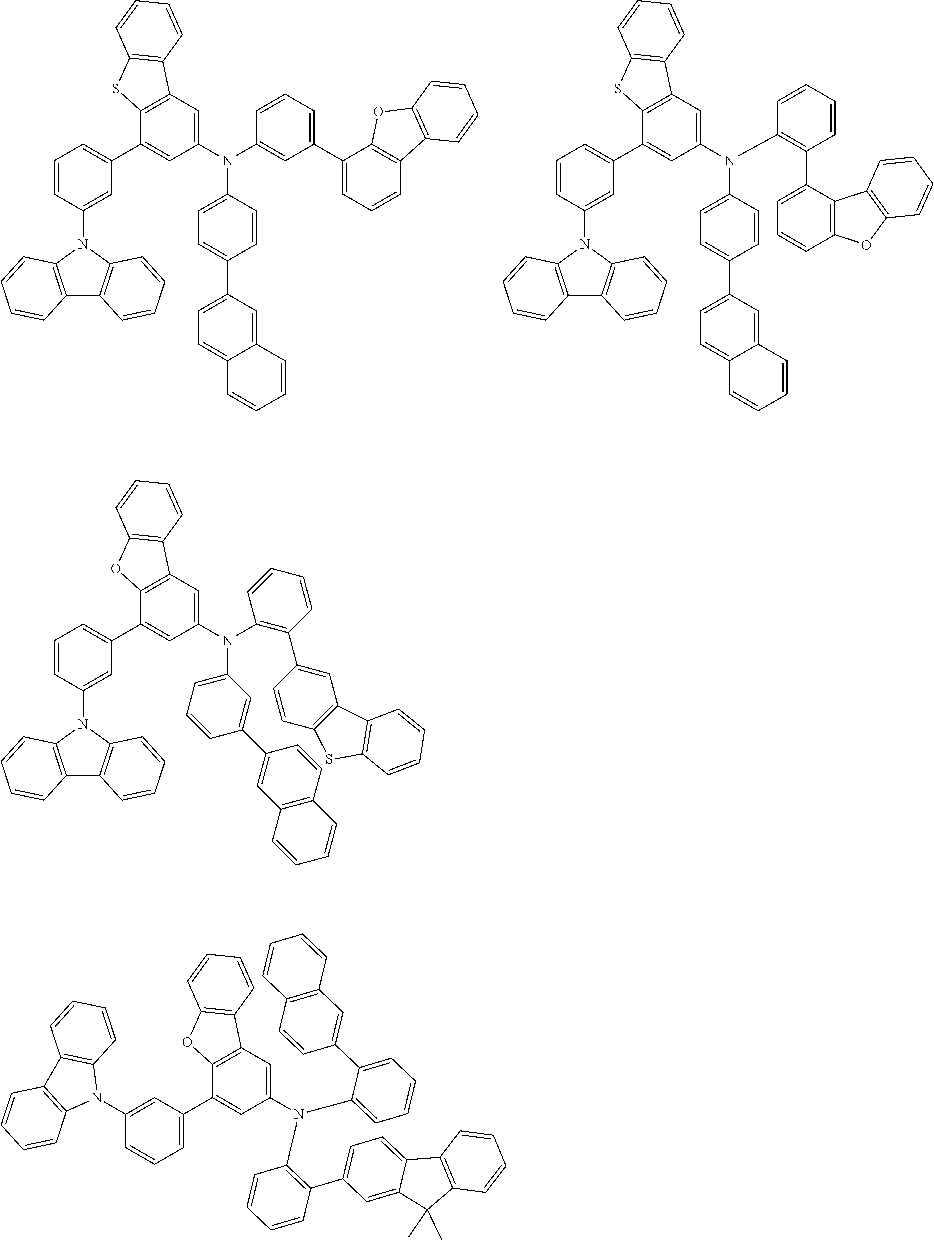 [SMILES: c1cc(-c2cccc3c2oc2ccccc23)cc(N(c2ccc(-c3ccc4ccccc4c3)cc2)c2cc(-c3cccc(-n4c5ccccc5c5ccccc54)c3)c3sc4ccccc4c3c2)c1 ; c1cc(-c2cc(N(c3ccc(-c4ccc5ccccc5c4)cc3)c3ccccc3-c3cccc4oc5ccccc5c34)cc3c2sc2ccccc23)cc(-n2c3ccccc3c3ccccc32)c1 ; c1cc(-c2ccc3ccccc3c2)cc(N(c2cc(-c3cccc(-n4c5ccccc5c5ccccc54)c3)c3oc4ccccc4c3c2)c2ccccc2-c2ccc3sc4ccccc4c3c2)c1 ; CC1(C)c2ccccc2-c2ccc(-c3ccccc3N(c3cc(-c4cccc(-n5c6ccccc6c6ccccc65)c4)c4oc5ccccc5c4c3)c3ccccc3-c3ccc4ccccc4c3)cc21]

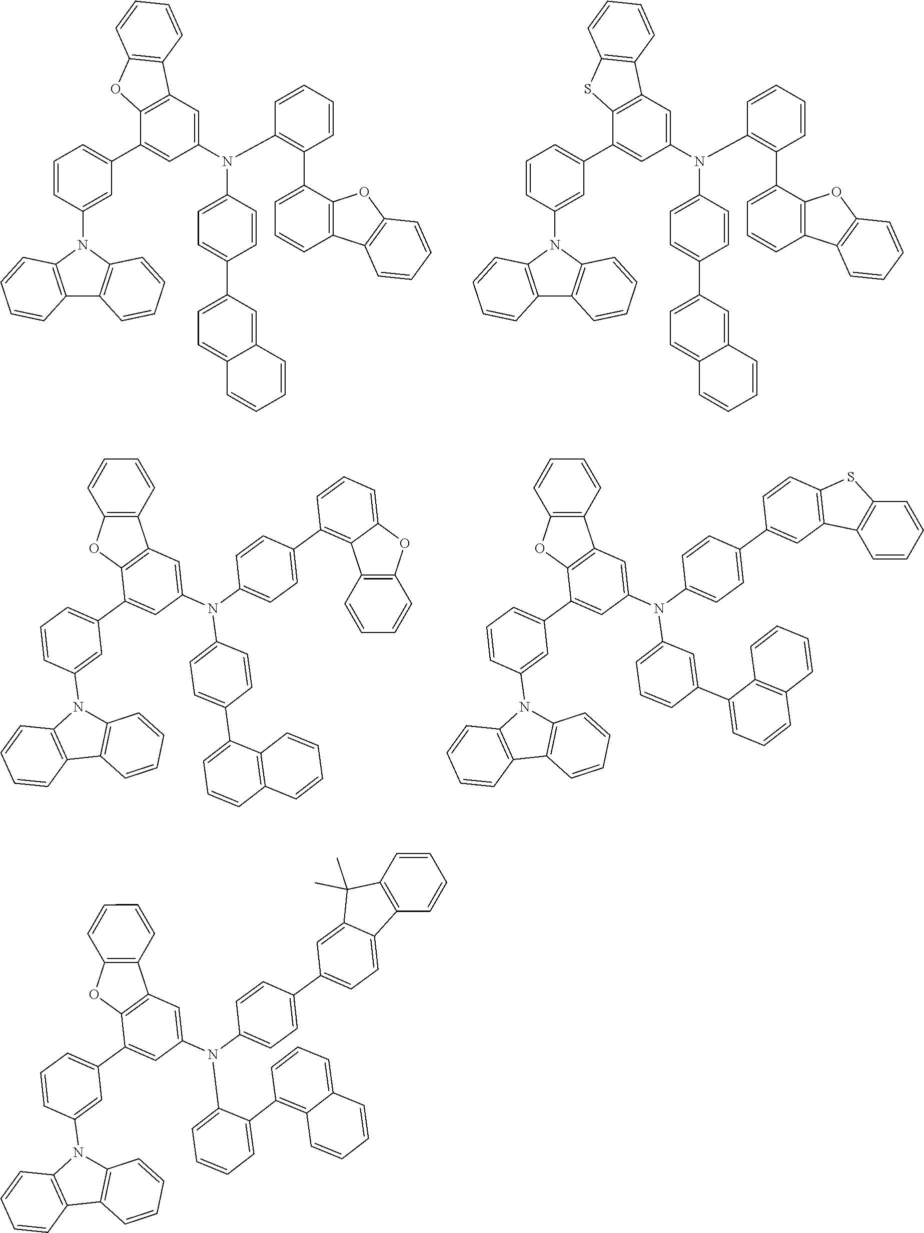

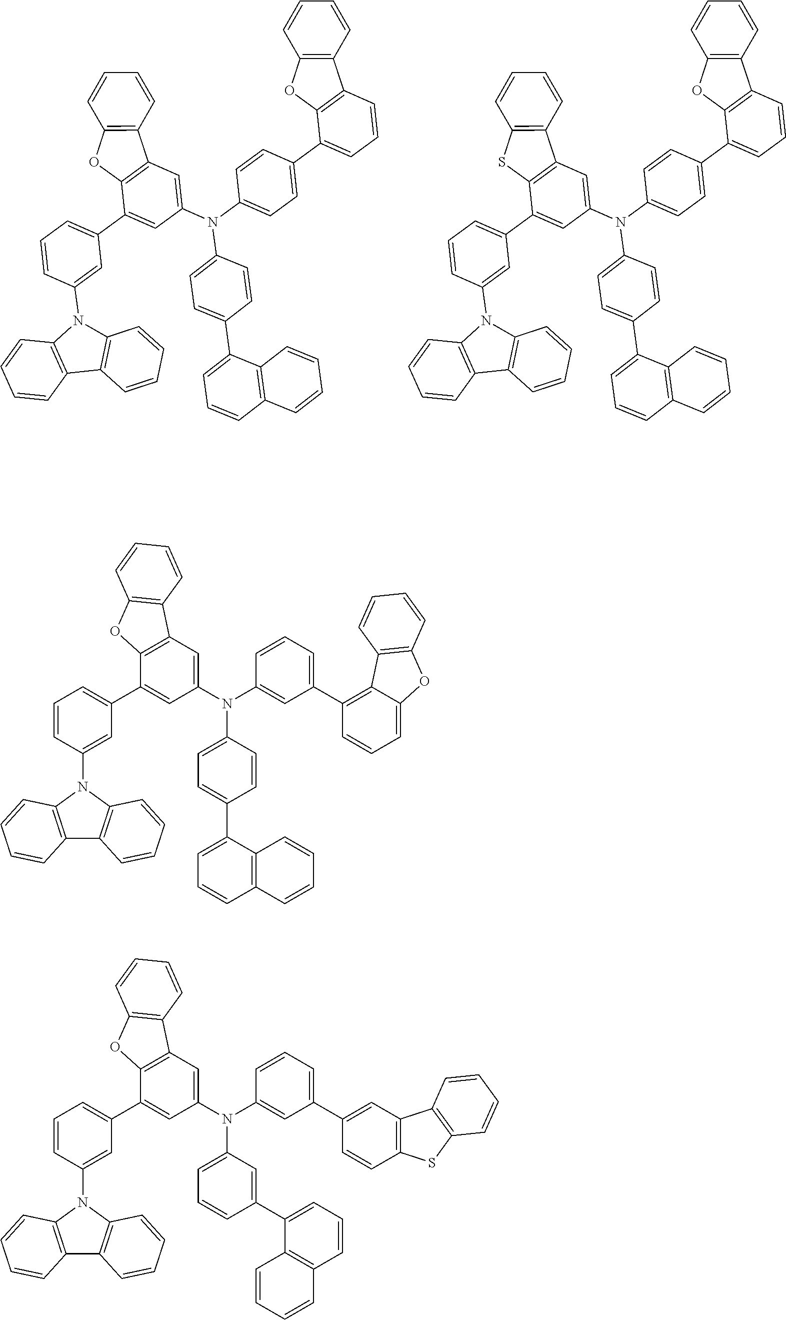 [SMILES: c1cc(-c2cc(N(c3ccc(-c4cccc5ccccc45)cc3)c3ccc(-c4cccc5c4oc4ccccc45)cc3)cc3c2oc2ccccc23)cc(-n2c3ccccc3c3ccccc32)c1 ; c1cc(-c2cc(N(c3ccc(-c4cccc5ccccc45)cc3)c3ccc(-c4cccc5c4oc4ccccc45)cc3)cc3c2sc2ccccc23)cc(-n2c3ccccc3c3ccccc32)c1 ; c1cc(-c2cccc3oc4ccccc4c23)cc(N(c2ccc(-c3cccc4ccccc34)cc2)c2cc(-c3cccc(-n4c5ccccc5c5ccccc54)c3)c3oc4ccccc4c3c2)c1 ; c1cc(-c2ccc3sc4ccccc4c3c2)cc(N(c2cccc(-c3cccc4ccccc34)c2)c2cc(-c3cccc(-n4c5ccccc5c5ccccc54)c3)c3oc4ccccc4c3c2)c1]

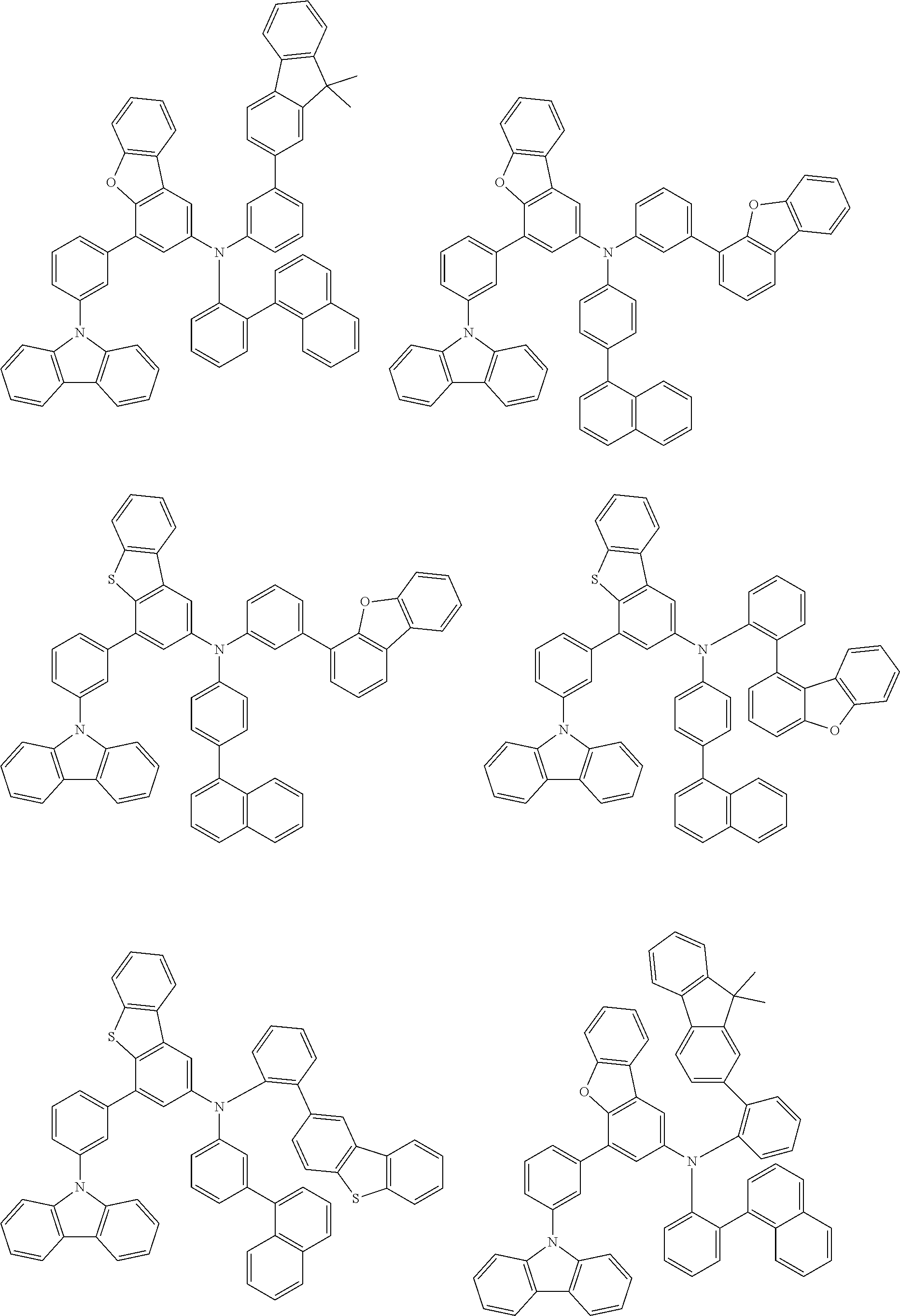

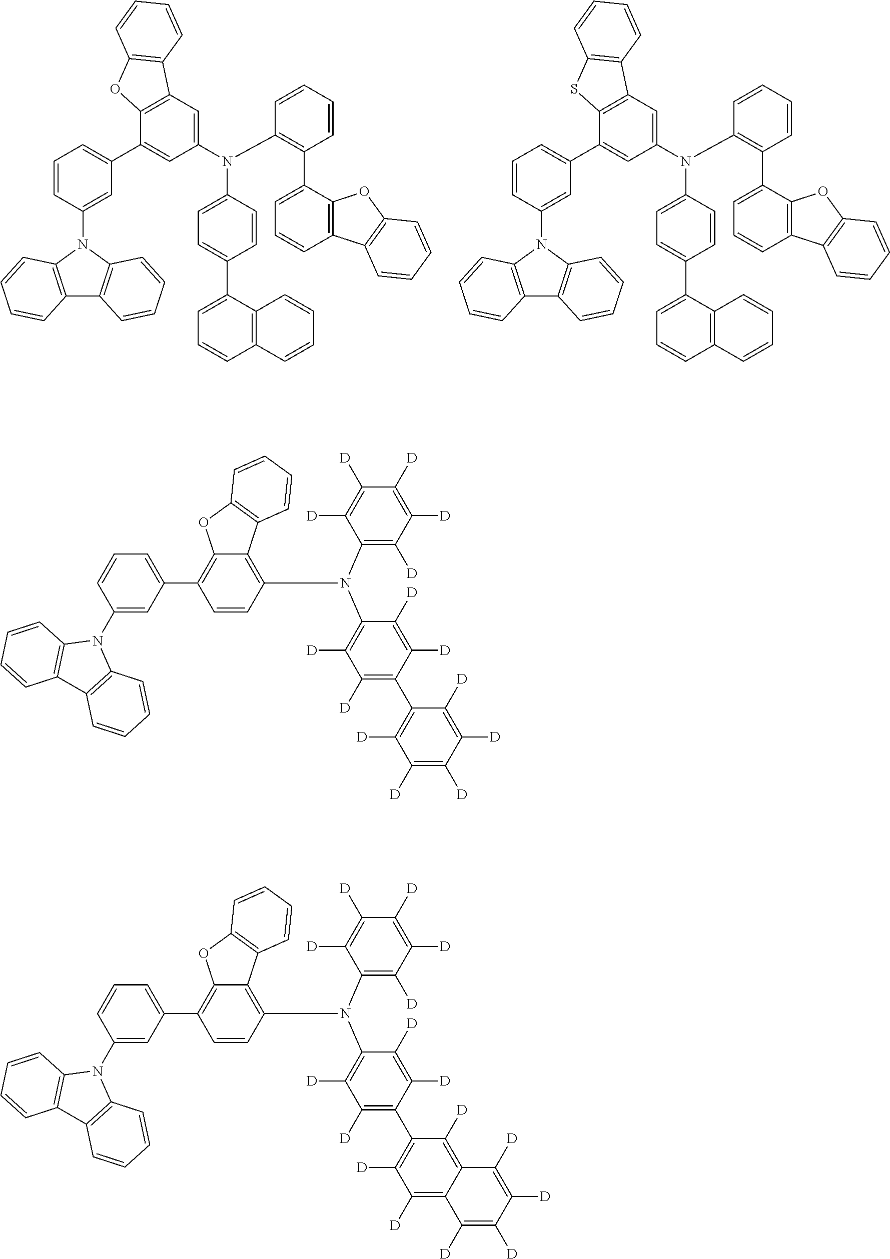

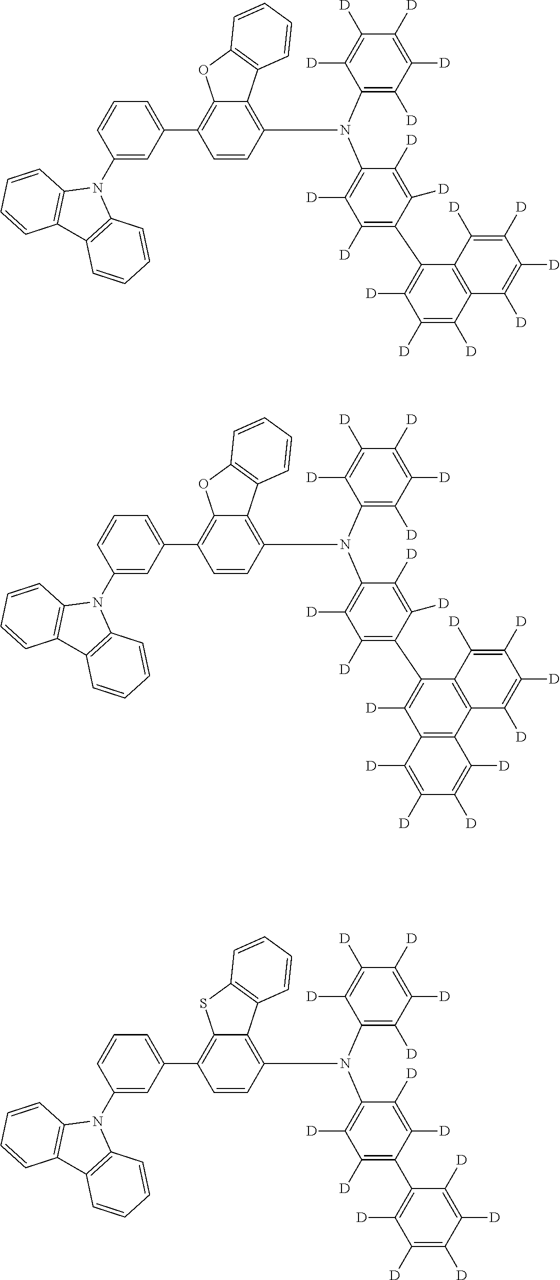

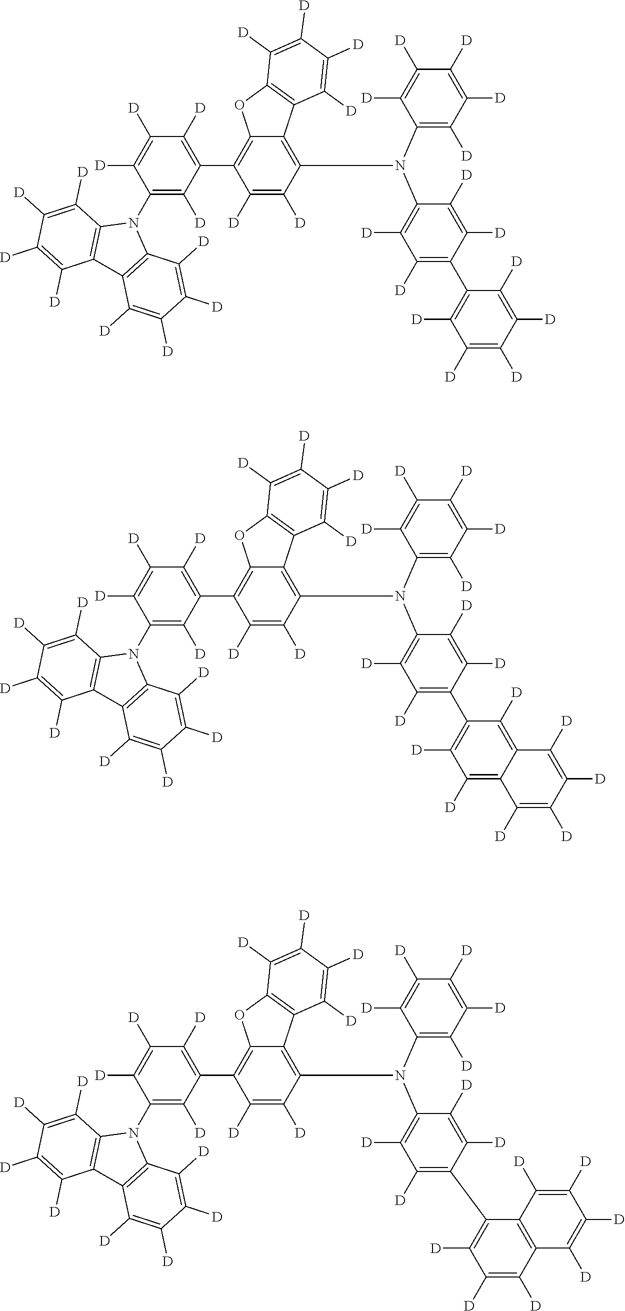

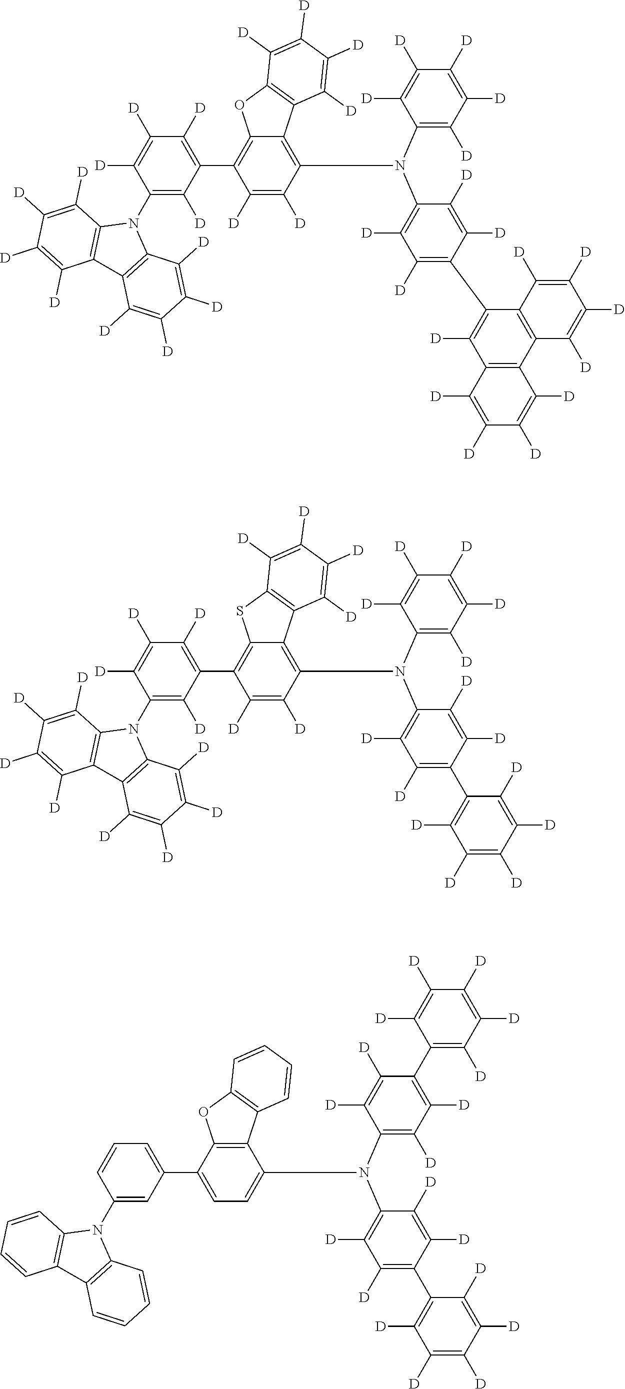

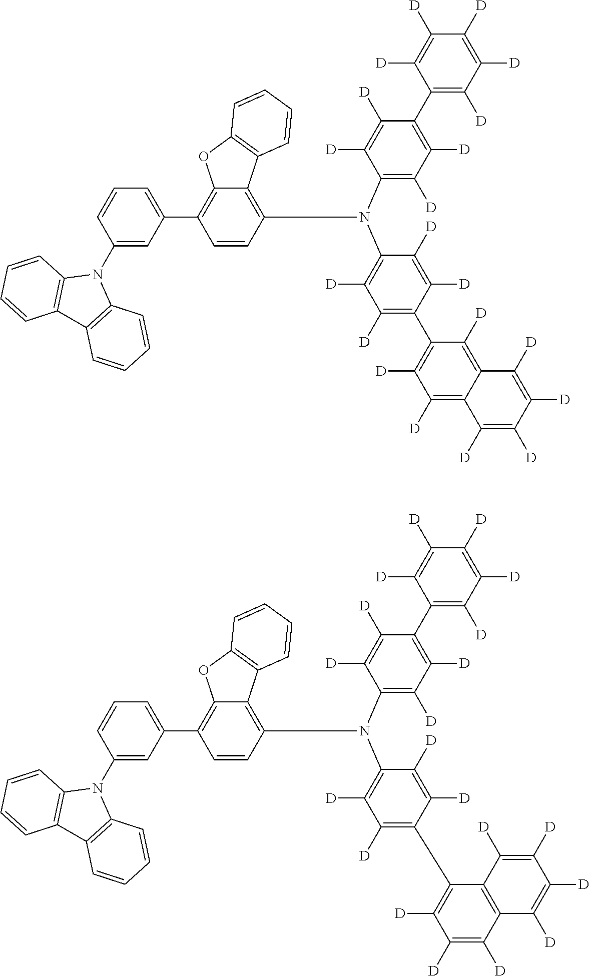 [SMILES: [2H]c1c([2H])c([2H])c(-c2c([2H])c([2H])c(N(c3c([2H])c([2H])c(-c4c([2H])c([2H])c5c([2H])c([2H])c([2H])c([2H])c5c4[2H])c([2H])c3[2H])c3ccc(-c4cccc(-n5c6ccccc6c6ccccc65)c4)c4oc5ccccc5c34)c([2H])c2[2H])c([2H])c1[2H] ; [2H]c1c([2H])c([2H])c(-c2c([2H])c([2H])c(N(c3c([2H])c([2H])c(-c4c([2H])c([2H])c([2H])c5c([2H])c([2H])c([2H])c([2H])c45)c([2H])c3[2H])c3ccc(-c4cccc(-n5c6ccccc6c6ccccc65)c4)c4oc5ccccc5c34)c([2H])c2[2H])c([2H])c1[2H]]

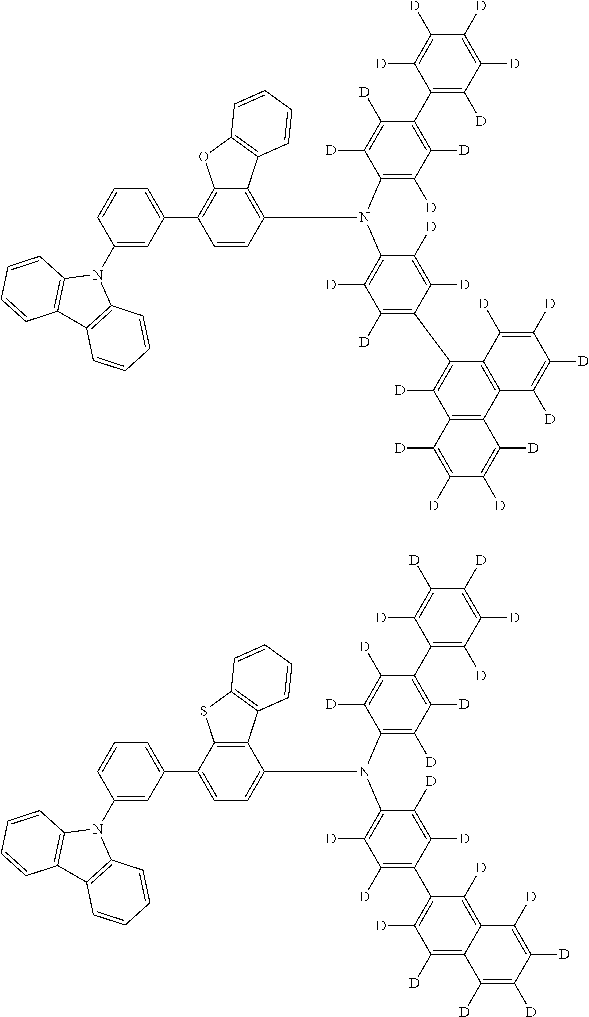 [SMILES: [2H]c1c([2H])c([2H])c(-c2c([2H])c([2H])c(N(c3c([2H])c([2H])c(-c4c([2H])c5c([2H])c([2H])c([2H])c([2H])c5c5c([2H])c([2H])c([2H])c([2H])c45)c([2H])c3[2H])c3ccc(-c4cccc(-n5c6ccccc6c6ccccc65)c4)c4oc5ccccc5c34)c([2H])c2[2H])c([2H])c1[2H] ; [2H]c1c([2H])c([2H])c(-c2c([2H])c([2H])c(N(c3c([2H])c([2H])c(-c4c([2H])c([2H])c5c([2H])c([2H])c([2H])c([2H])c5c4[2H])c([2H])c3[2H])c3ccc(-c4cccc(-n5c6ccccc6c6ccccc65)c4)c4sc5ccccc5c34)c([2H])c2[2H])c([2H])c1[2H]]

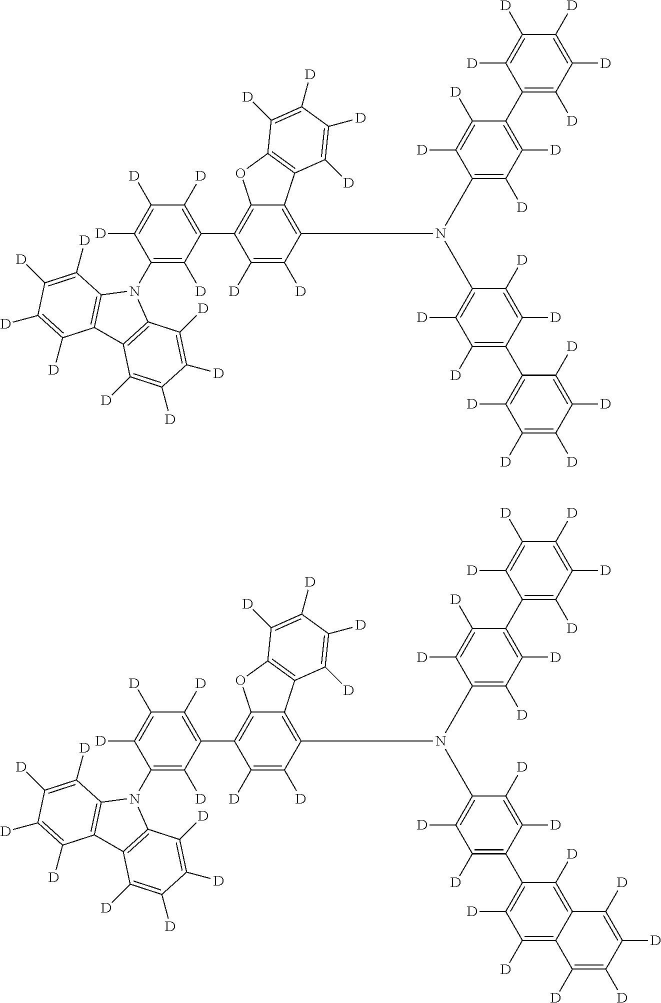

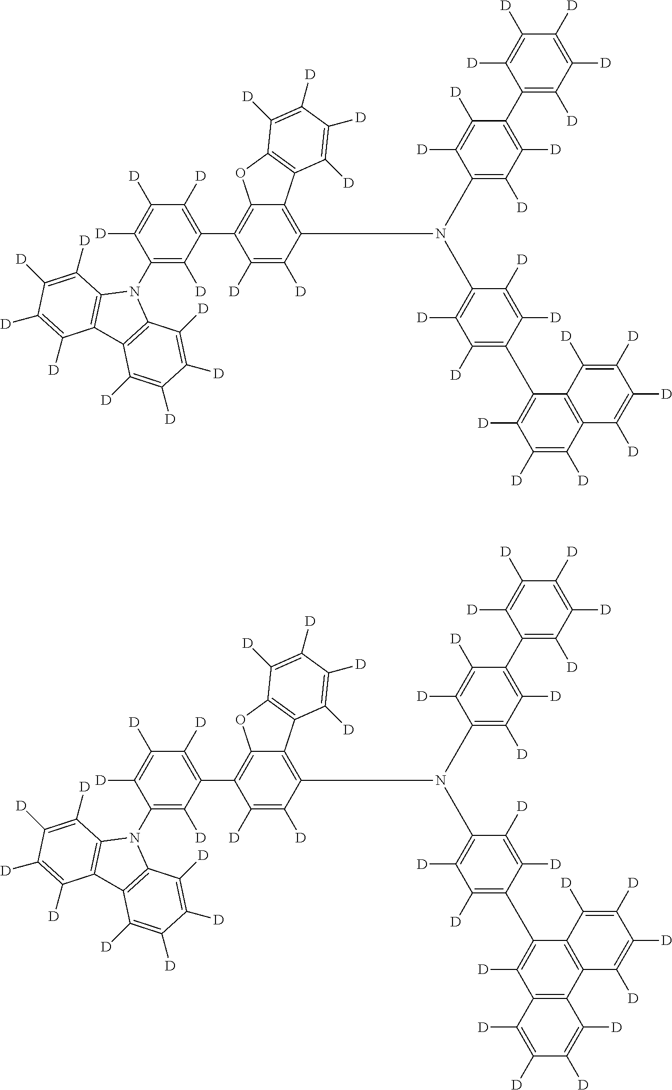

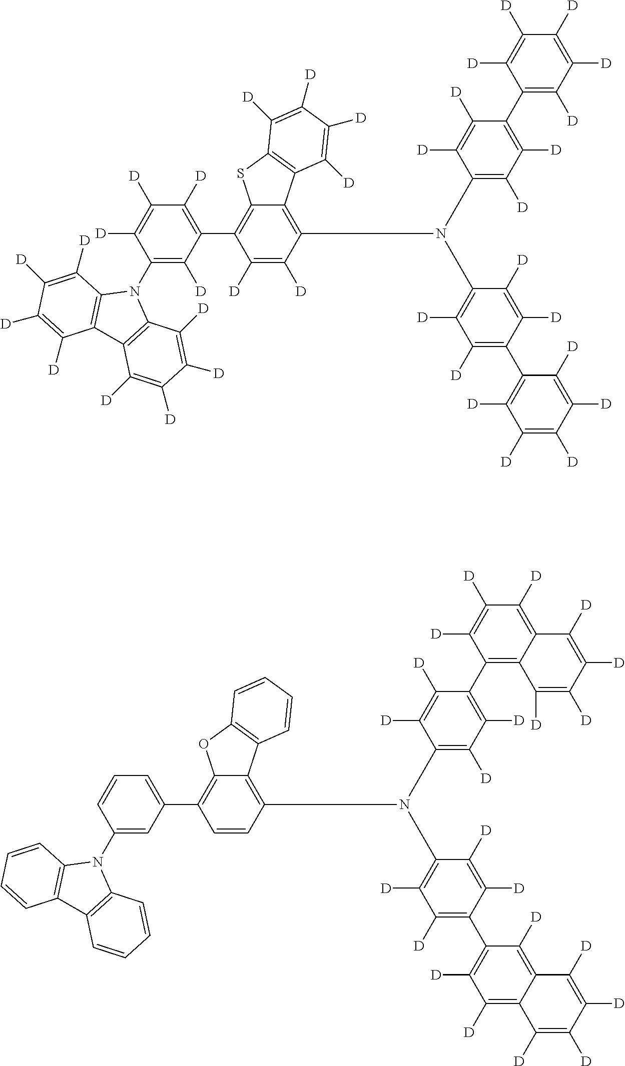

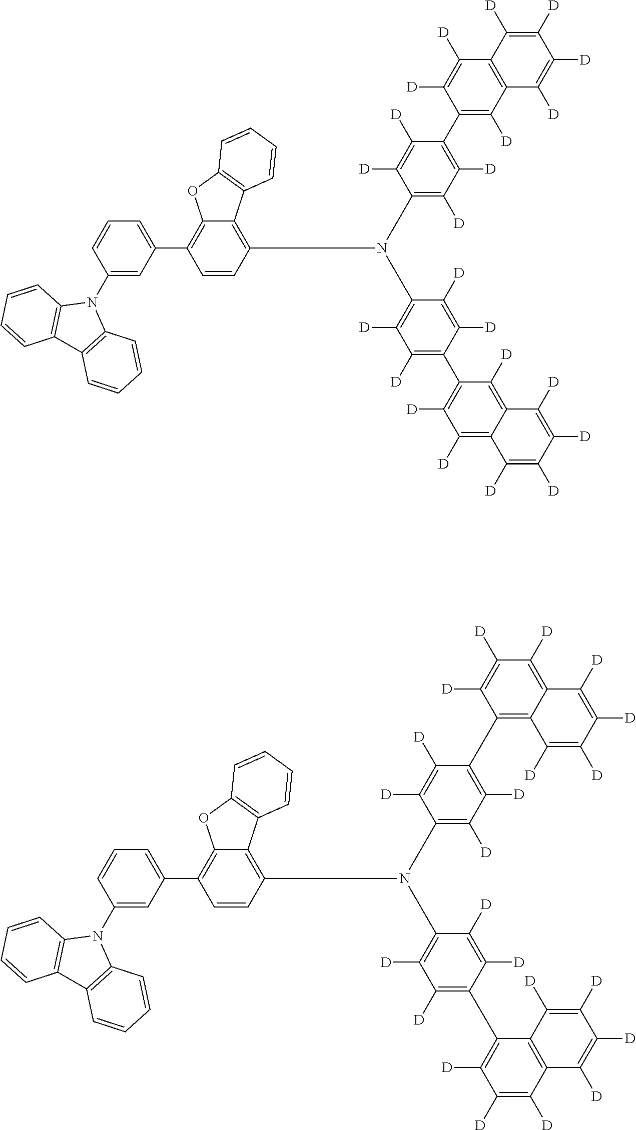 [SMILES: [2H]c1c([2H])c(N(c2c([2H])c([2H])c(-c3c([2H])c([2H])c4c([2H])c([2H])c([2H])c([2H])c4c3[2H])c([2H])c2[2H])c2ccc(-c3cccc(-n4c5ccccc5c5ccccc54)c3)c3oc4ccccc4c23)c([2H])c([2H])c1-c1c([2H])c([2H])c2c([2H])c([2H])c([2H])c([2H])c2c1[2H] ; [2H]c1c([2H])c(N(c2c([2H])c([2H])c(-c3c([2H])c([2H])c([2H])c4c([2H])c([2H])c([2H])c([2H])c34)c([2H])c2[2H])c2ccc(-c3cccc(-n4c5ccccc5c5ccccc54)c3)c3oc4ccccc4c23)c([2H])c([2H])c1-c1c([2H])c([2H])c([2H])c2c([2H])c([2H])c([2H])c([2H])c12]

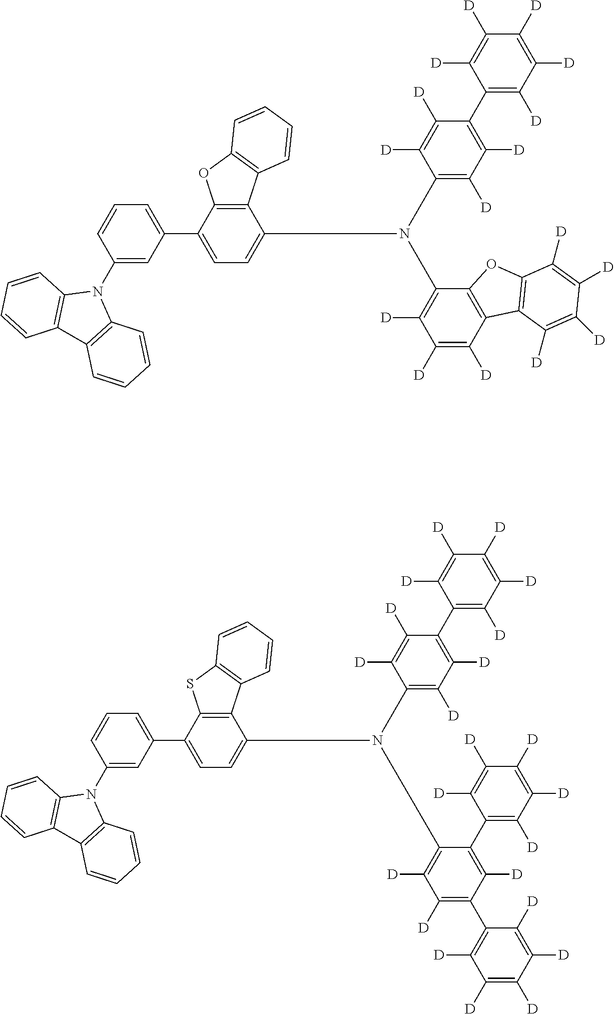 [SMILES: [2H]c1c([2H])c([2H])c(-c2c([2H])c([2H])c(N(c3c([2H])c([2H])c([2H])c4c3oc3c([2H])c([2H])c([2H])c([2H])c34)c3ccc(-c4cccc(-n5c6ccccc6c6ccccc65)c4)c4oc5ccccc5c34)c([2H])c2[2H])c([2H])c1[2H] ; [2H]c1c([2H])c([2H])c(-c2c([2H])c([2H])c(N(c3c([2H])c([2H])c(-c4c([2H])c([2H])c([2H])c([2H])c4[2H])c([2H])c3-c3c([2H])c([2H])c([2H])c([2H])c3[2H])c3ccc(-c4cccc(-n5c6ccccc6c6ccccc65)c4)c4sc5ccccc5c34)c([2H])c2[2H])c([2H])c1[2H]]

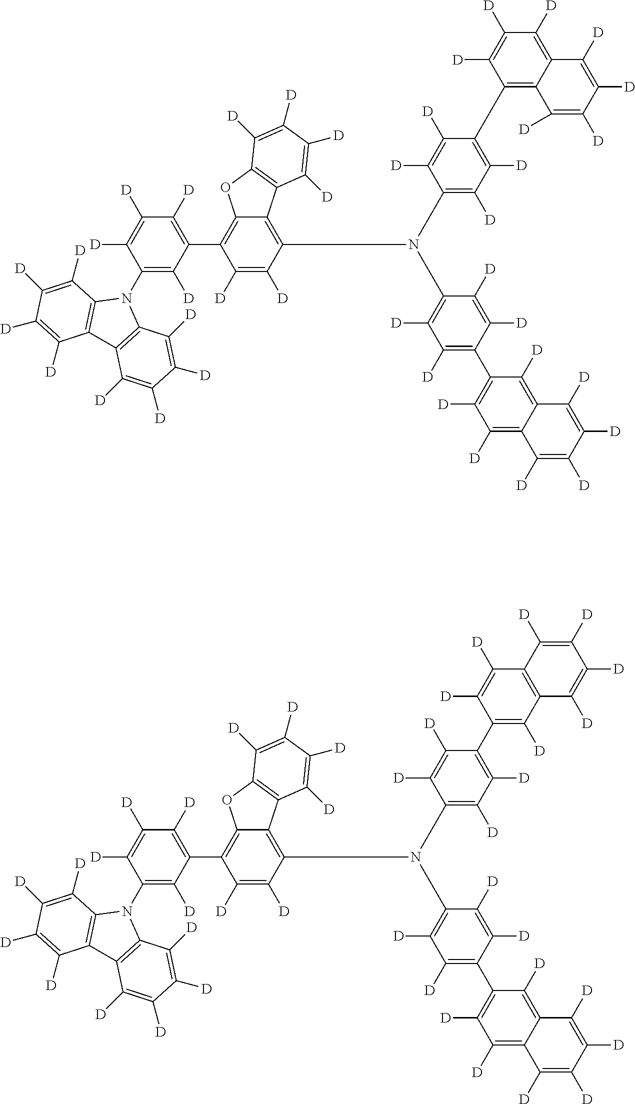

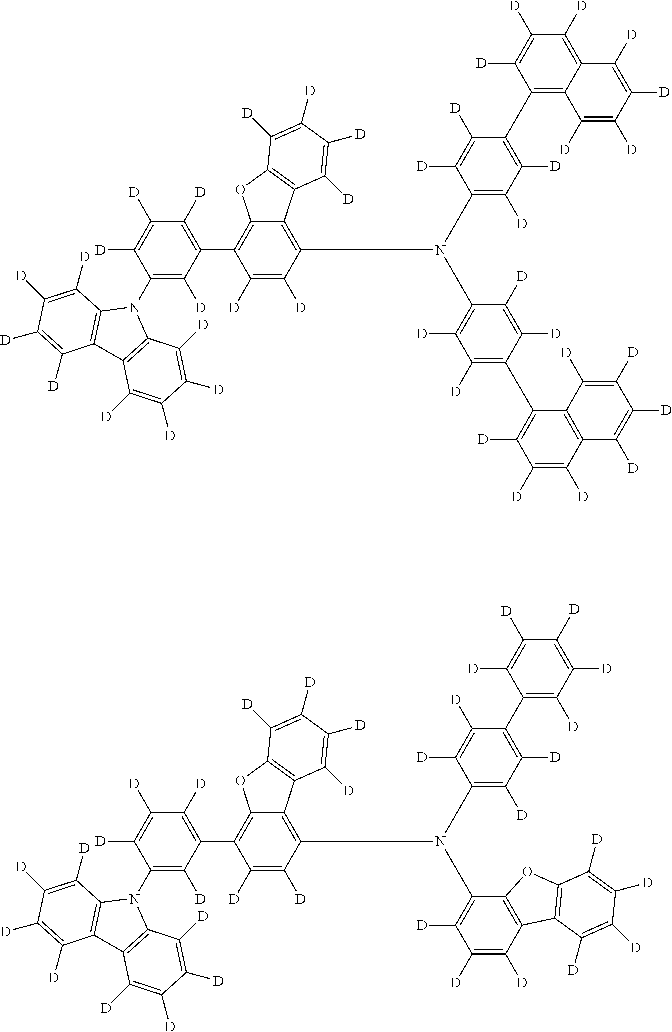

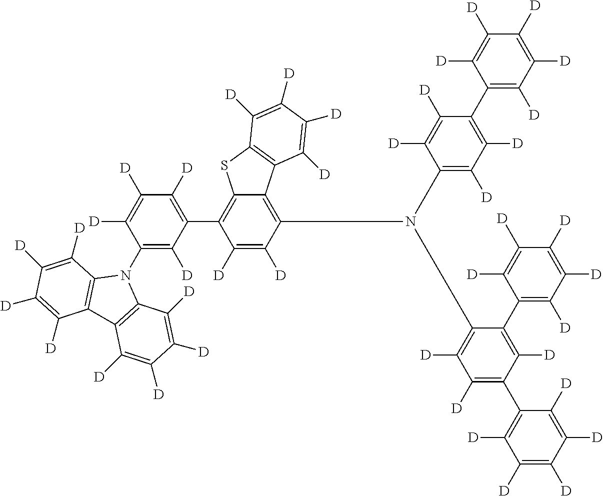 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3-c3c([2H])c([2H])c([2H])c([2H])c3[2H])c3c([2H])c([2H])c(-c4c([2H])c([2H])c([2H])c(-n5c6c([2H])c([2H])c([2H])c([2H])c6c6c([2H])c([2H])c([2H])c([2H])c65)c4[2H])c4sc5c([2H])c([2H])c([2H])c([2H])c5c34)c([2H])c2[2H])c([2H])c1[2H]]

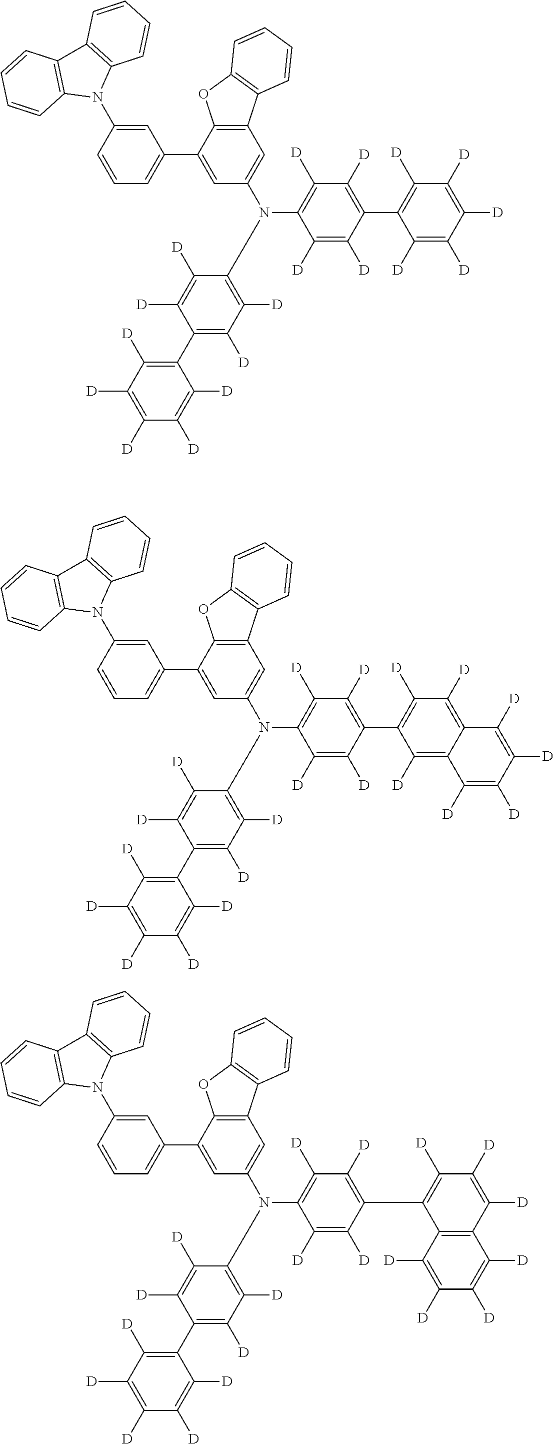

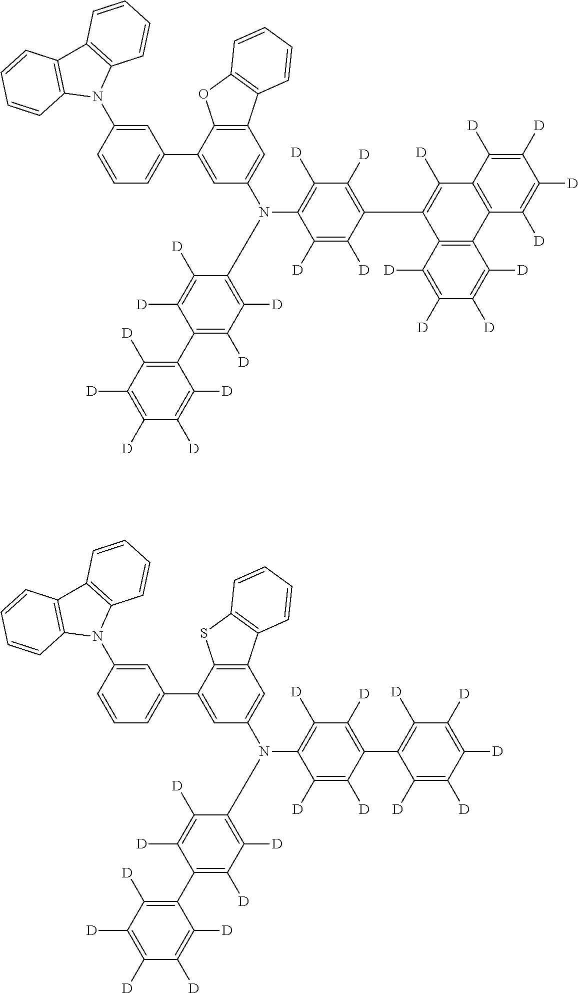 [SMILES: [2H]c1c([2H])c([2H])c(-c2c([2H])c([2H])c(N(c3cc(-c4cccc(-n5c6ccccc6c6ccccc65)c4)c4oc5ccccc5c4c3)c3c([2H])c([2H])c(-c4c([2H])c5c([2H])c([2H])c([2H])c([2H])c5c5c([2H])c([2H])c([2H])c([2H])c45)c([2H])c3[2H])c([2H])c2[2H])c([2H])c1[2H] ; [2H]c1c([2H])c([2H])c(-c2c([2H])c([2H])c(N(c3cc(-c4cccc(-n5c6ccccc6c6ccccc65)c4)c4sc5ccccc5c4c3)c3c([2H])c([2H])c(-c4c([2H])c([2H])c([2H])c([2H])c4[2H])c([2H])c3[2H])c([2H])c2[2H])c([2H])c1[2H]]

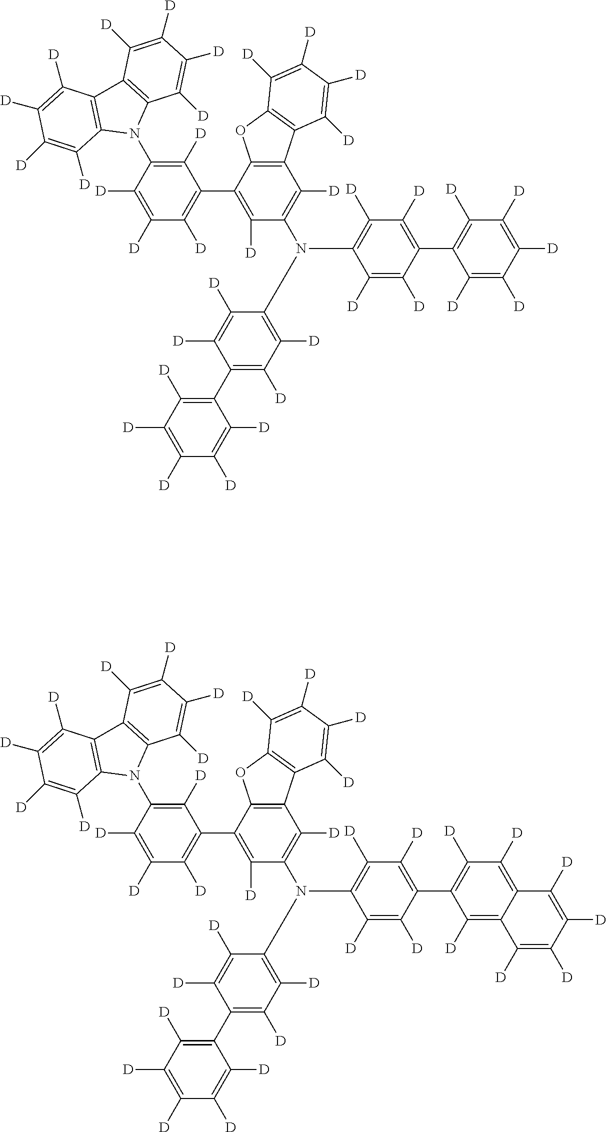

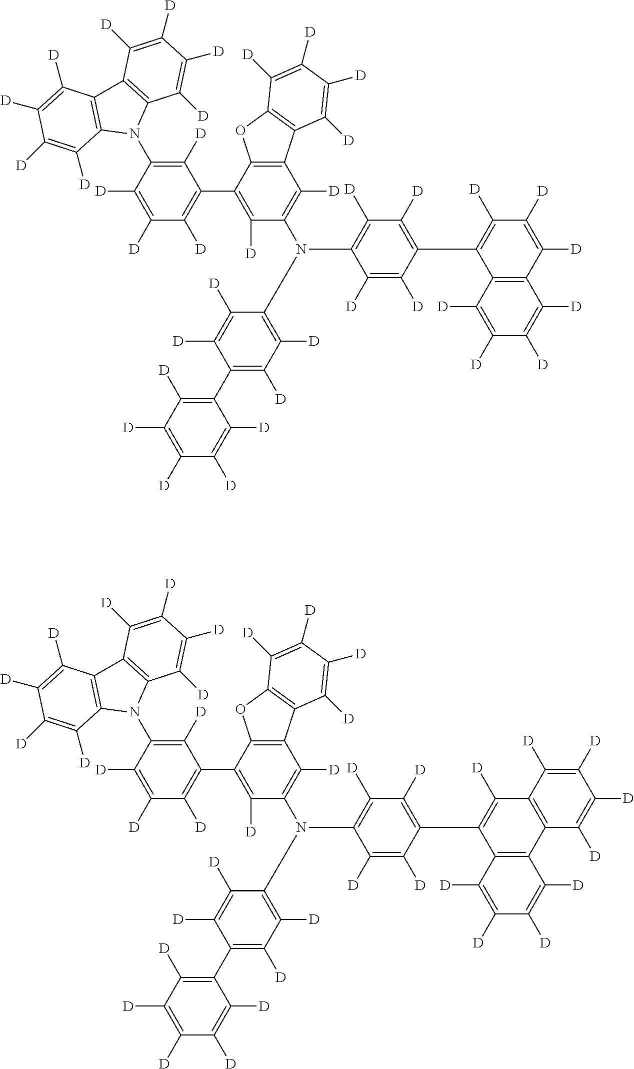

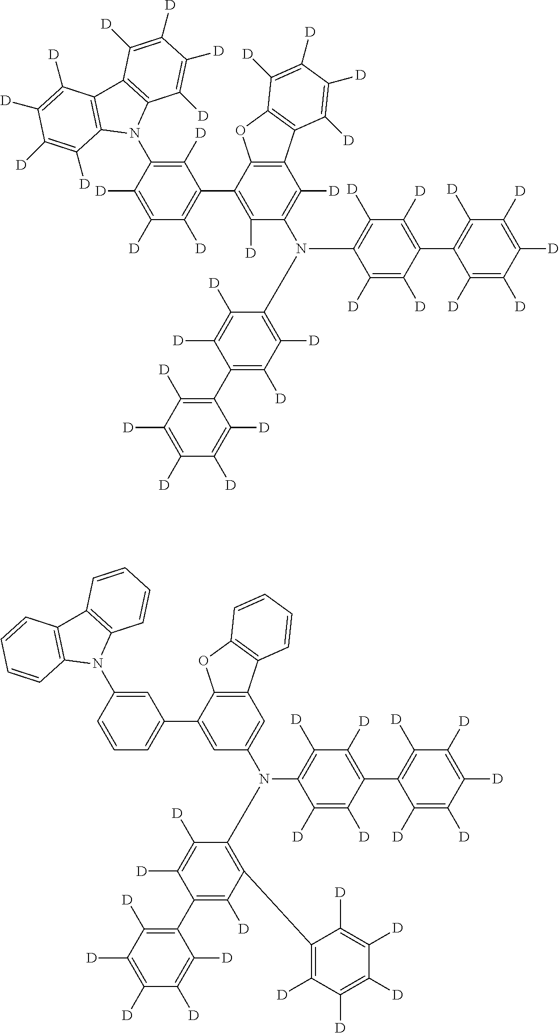

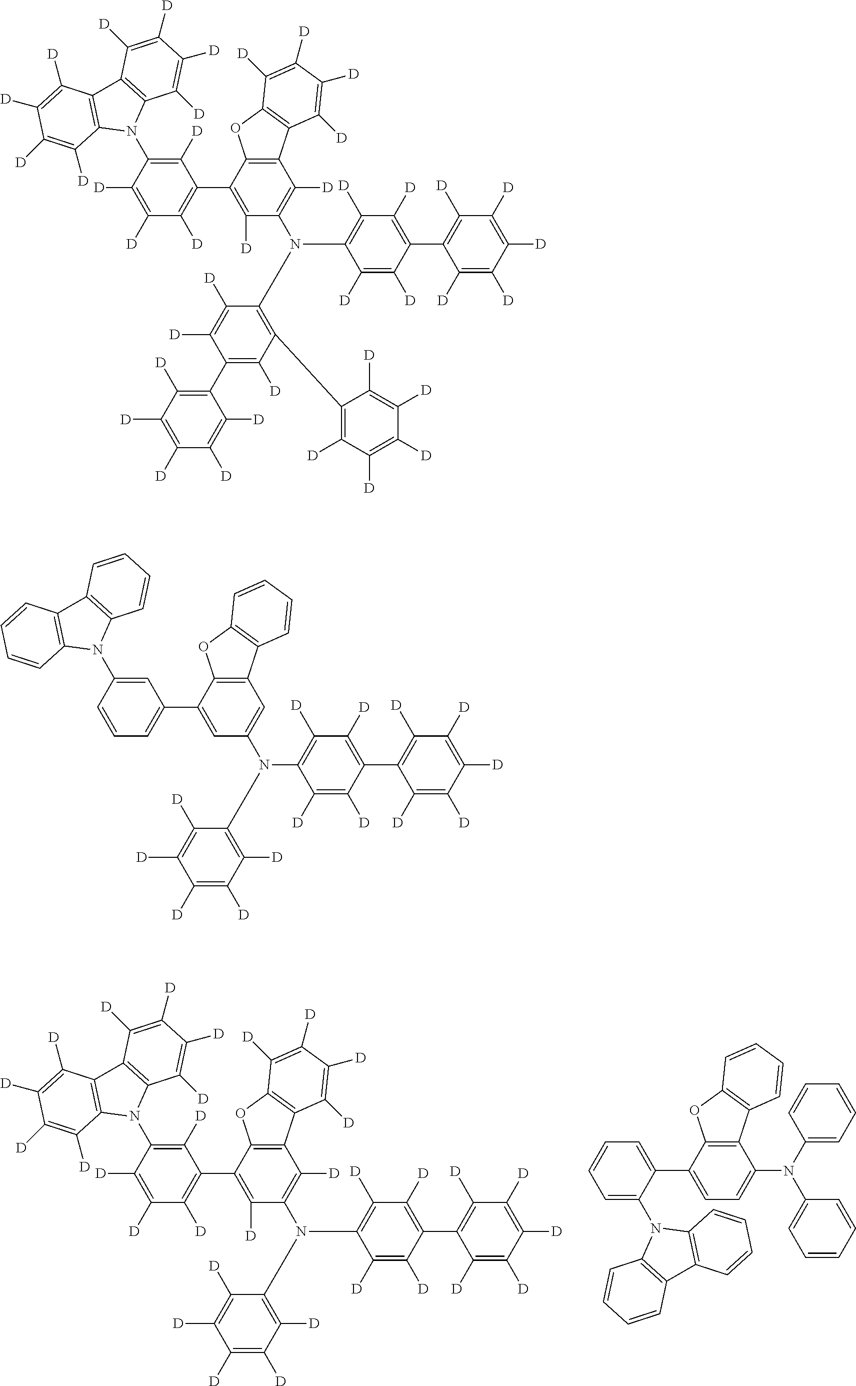

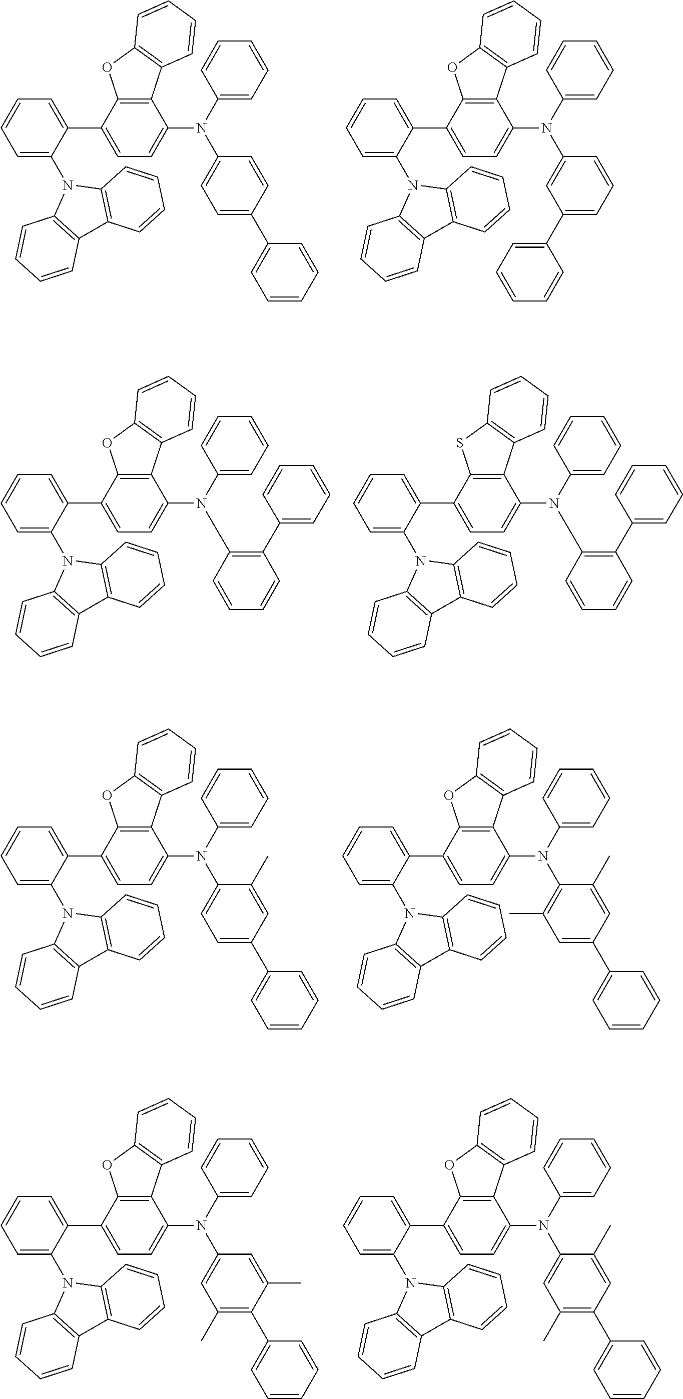

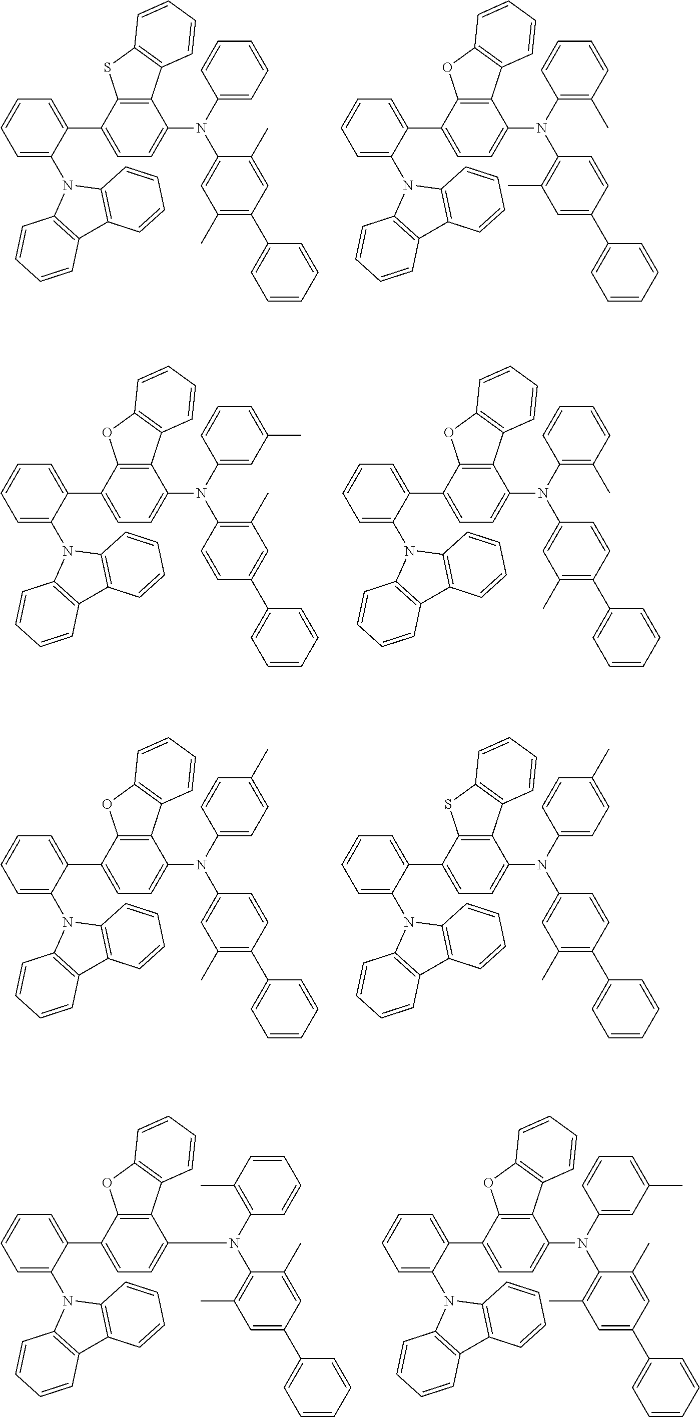

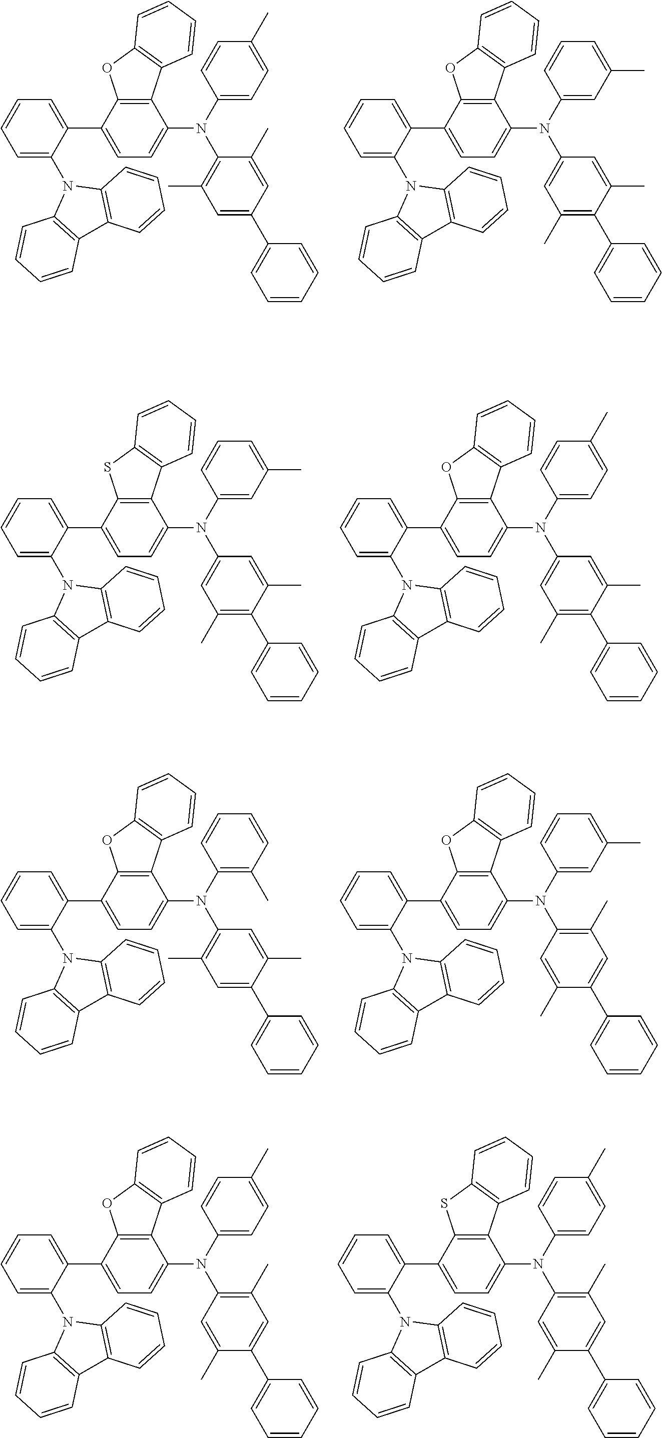

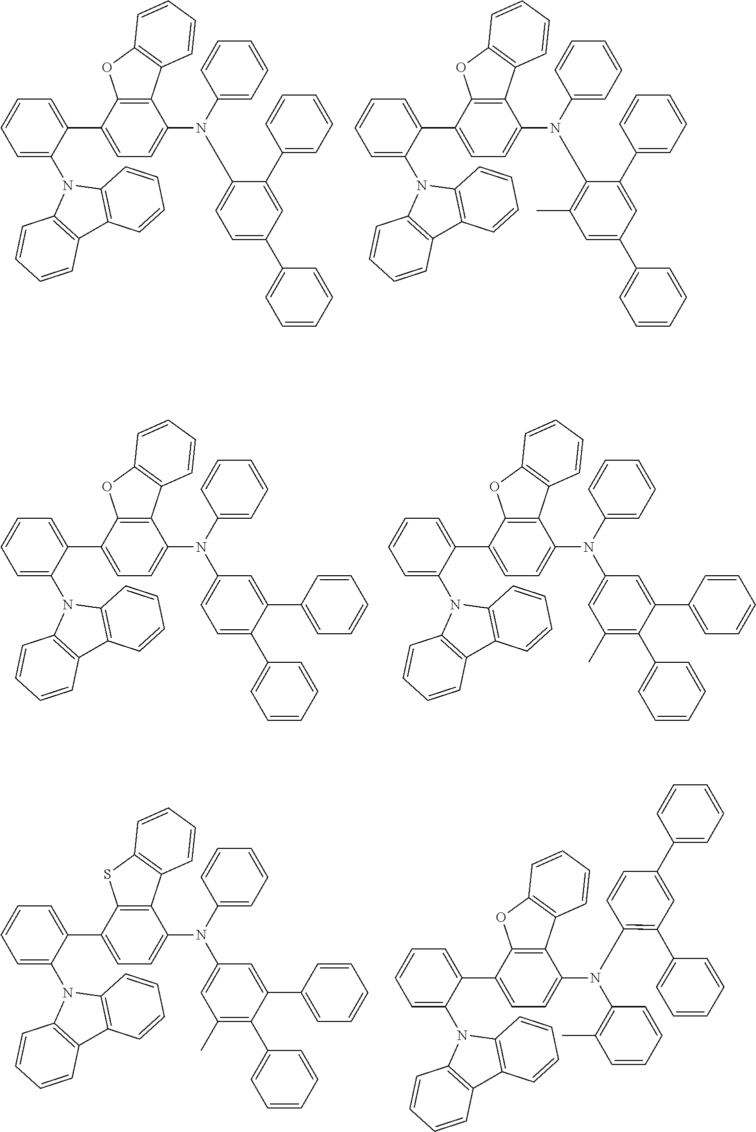

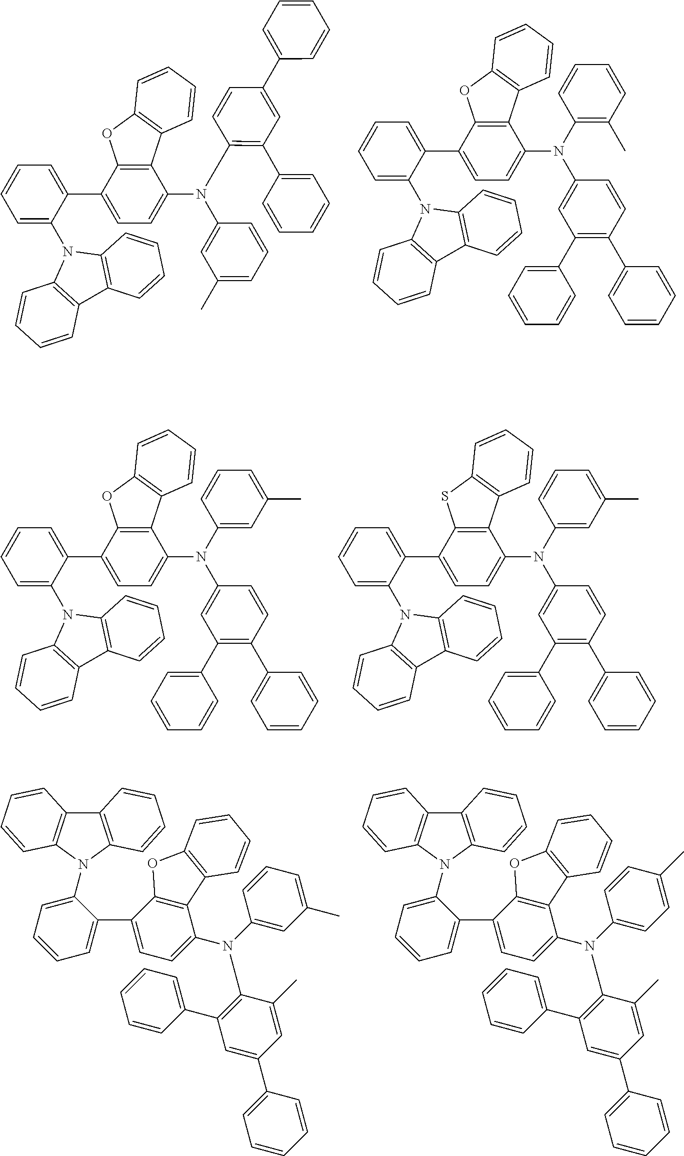

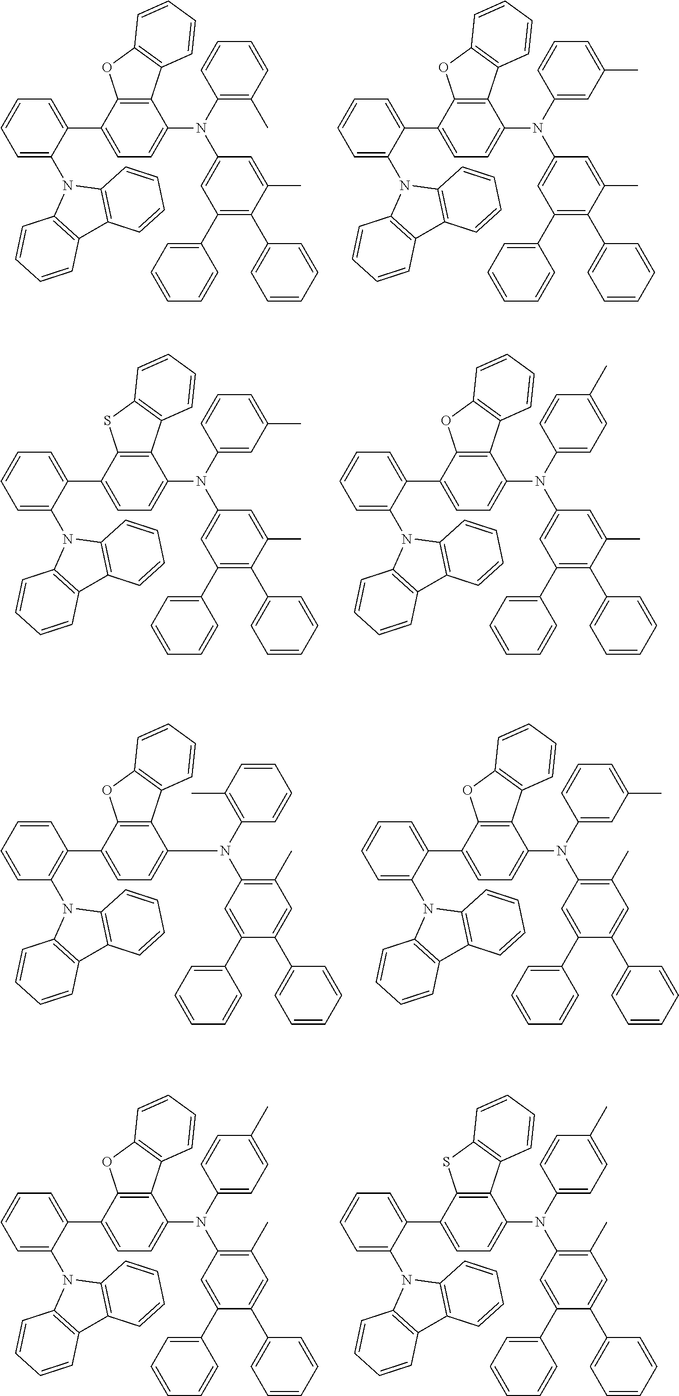

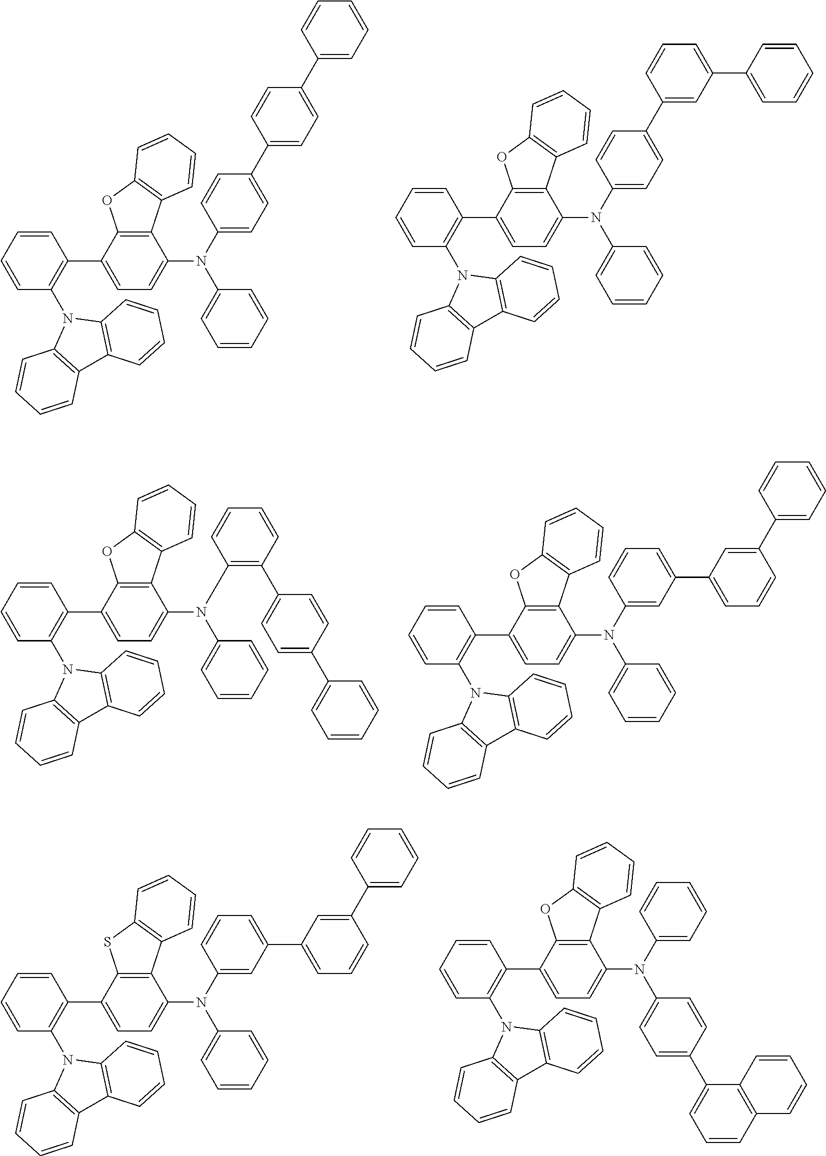

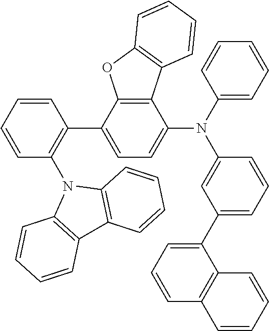 [SMILES: c1ccc(N(c2cccc(-c3cccc4ccccc34)c2)c2ccc(-c3ccccc3-n3c4ccccc4c4ccccc43)c3oc4ccccc4c23)cc1]

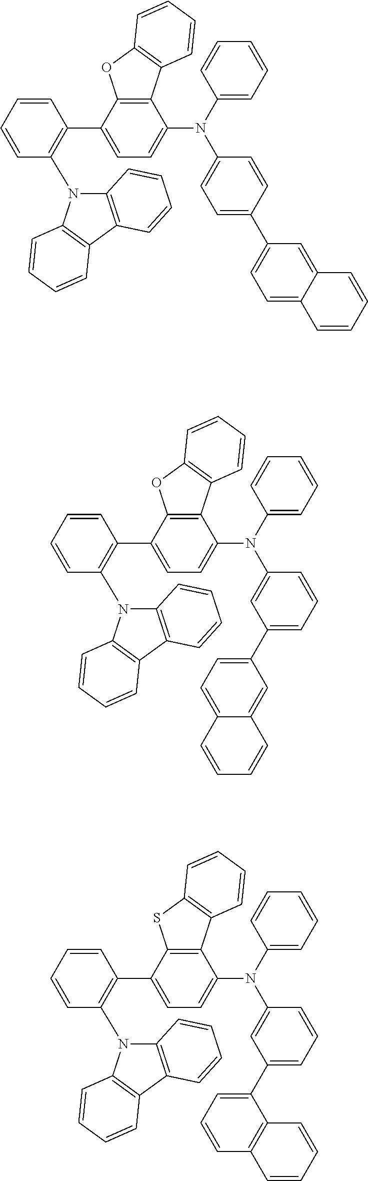 [SMILES: c1ccc(N(c2ccc(-c3ccc4ccccc4c3)cc2)c2ccc(-c3ccccc3-n3c4ccccc4c4ccccc43)c3oc4ccccc4c23)cc1 ; c1ccc(N(c2cccc(-c3ccc4ccccc4c3)c2)c2ccc(-c3ccccc3-n3c4ccccc4c4ccccc43)c3oc4ccccc4c23)cc1 ; c1ccc(N(c2cccc(-c3cccc4ccccc34)c2)c2ccc(-c3ccccc3-n3c4ccccc4c4ccccc43)c3sc4ccccc4c23)cc1]

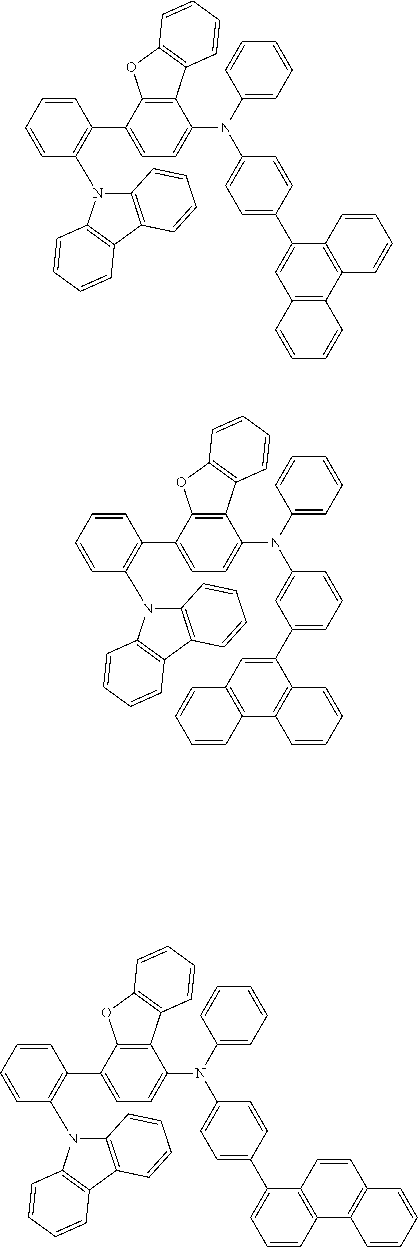 [SMILES: c1ccc(N(c2ccc(-c3cc4ccccc4c4ccccc34)cc2)c2ccc(-c3ccccc3-n3c4ccccc4c4ccccc43)c3oc4ccccc4c23)cc1 ; c1ccc(N(c2cccc(-c3cc4ccccc4c4ccccc34)c2)c2ccc(-c3ccccc3-n3c4ccccc4c4ccccc43)c3oc4ccccc4c23)cc1 ; c1ccc(N(c2ccc(-c3cccc4c3ccc3ccccc34)cc2)c2ccc(-c3ccccc3-n3c4ccccc4c4ccccc43)c3oc4ccccc4c23)cc1]

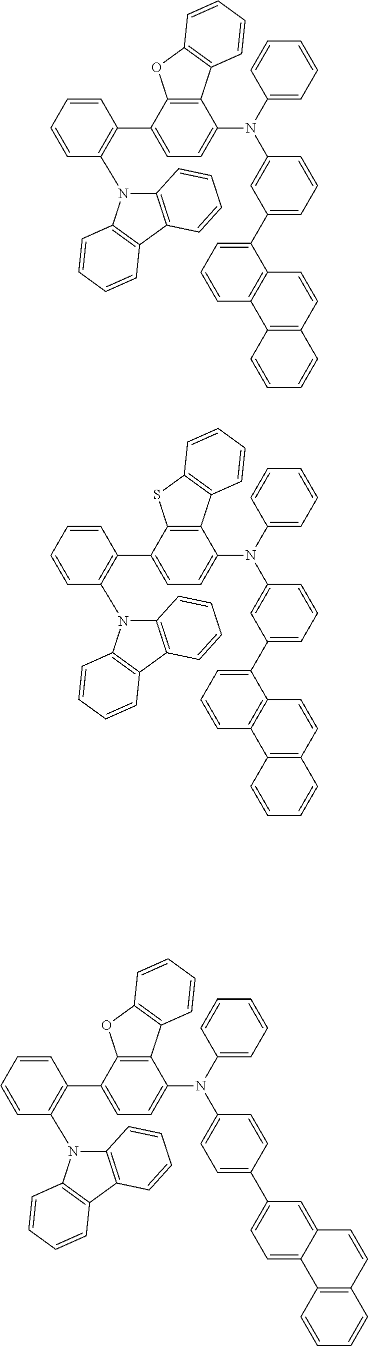 [SMILES: c1ccc(N(c2cccc(-c3cccc4c3ccc3ccccc34)c2)c2ccc(-c3ccccc3-n3c4ccccc4c4ccccc43)c3oc4ccccc4c23)cc1 ; c1ccc(N(c2cccc(-c3cccc4c3ccc3ccccc34)c2)c2ccc(-c3ccccc3-n3c4ccccc4c4ccccc43)c3sc4ccccc4c23)cc1 ; c1ccc(N(c2ccc(-c3ccc4c(ccc5ccccc54)c3)cc2)c2ccc(-c3ccccc3-n3c4ccccc4c4ccccc43)c3oc4ccccc4c23)cc1]

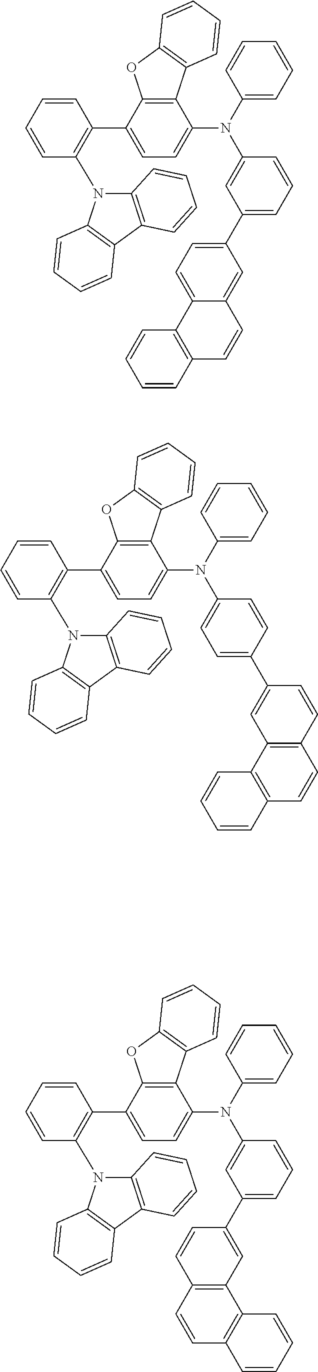 [SMILES: c1ccc(N(c2cccc(-c3ccc4c(ccc5ccccc54)c3)c2)c2ccc(-c3ccccc3-n3c4ccccc4c4ccccc43)c3oc4ccccc4c23)cc1 ; c1ccc(N(c2ccc(-c3ccc4ccc5ccccc5c4c3)cc2)c2ccc(-c3ccccc3-n3c4ccccc4c4ccccc43)c3oc4ccccc4c23)cc1 ; c1ccc(N(c2cccc(-c3ccc4ccc5ccccc5c4c3)c2)c2ccc(-c3ccccc3-n3c4ccccc4c4ccccc43)c3oc4ccccc4c23)cc1]

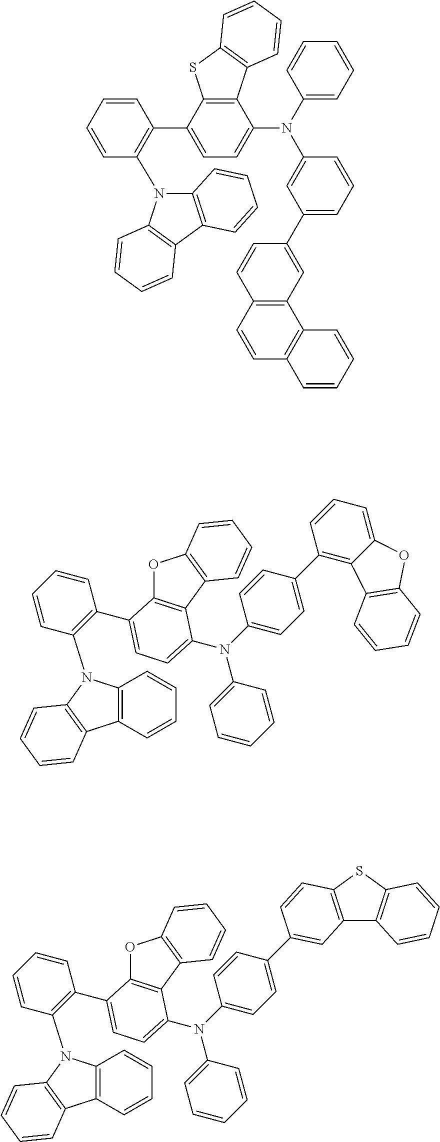 [SMILES: c1ccc(N(c2cccc(-c3ccc4ccc5ccccc5c4c3)c2)c2ccc(-c3ccccc3-n3c4ccccc4c4ccccc43)c3sc4ccccc4c23)cc1 ; c1ccc(N(c2ccc(-c3cccc4oc5ccccc5c34)cc2)c2ccc(-c3ccccc3-n3c4ccccc4c4ccccc43)c3oc4ccccc4c23)cc1 ; c1ccc(N(c2ccc(-c3ccc4sc5ccccc5c4c3)cc2)c2ccc(-c3ccccc3-n3c4ccccc4c4ccccc43)c3oc4ccccc4c23)cc1]

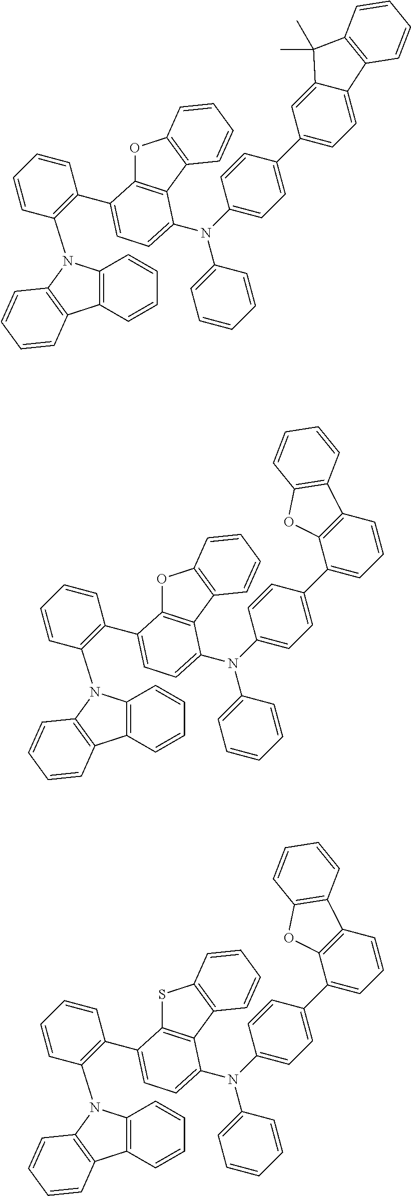 [SMILES: CC1(C)c2ccccc2-c2ccc(-c3ccc(N(c4ccccc4)c4ccc(-c5ccccc5-n5c6ccccc6c6ccccc65)c5oc6ccccc6c45)cc3)cc21 ; c1ccc(N(c2ccc(-c3cccc4c3oc3ccccc34)cc2)c2ccc(-c3ccccc3-n3c4ccccc4c4ccccc43)c3oc4ccccc4c23)cc1 ; c1ccc(N(c2ccc(-c3cccc4c3oc3ccccc34)cc2)c2ccc(-c3ccccc3-n3c4ccccc4c4ccccc43)c3sc4ccccc4c23)cc1]

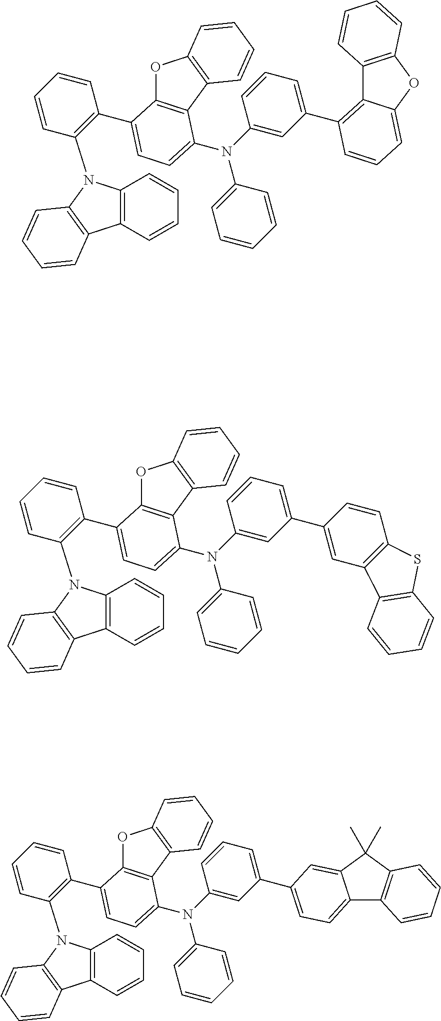 [SMILES: c1ccc(N(c2cccc(-c3cccc4oc5ccccc5c34)c2)c2ccc(-c3ccccc3-n3c4ccccc4c4ccccc43)c3oc4ccccc4c23)cc1 ; c1ccc(N(c2cccc(-c3ccc4sc5ccccc5c4c3)c2)c2ccc(-c3ccccc3-n3c4ccccc4c4ccccc43)c3oc4ccccc4c23)cc1 ; CC1(C)c2ccccc2-c2ccc(-c3cccc(N(c4ccccc4)c4ccc(-c5ccccc5-n5c6ccccc6c6ccccc65)c5oc6ccccc6c45)c3)cc21]

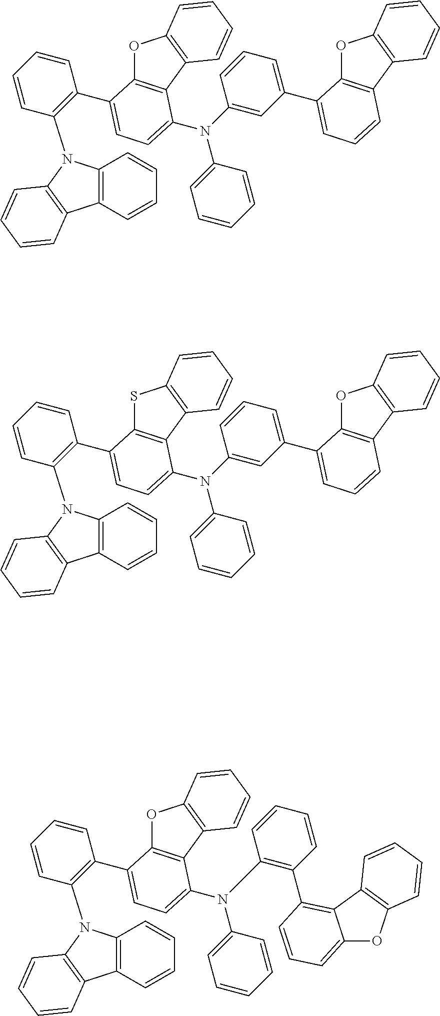 [SMILES: c1ccc(N(c2cccc(-c3cccc4c3oc3ccccc34)c2)c2ccc(-c3ccccc3-n3c4ccccc4c4ccccc43)c3oc4ccccc4c23)cc1 ; c1ccc(N(c2cccc(-c3cccc4c3oc3ccccc34)c2)c2ccc(-c3ccccc3-n3c4ccccc4c4ccccc43)c3sc4ccccc4c23)cc1 ; c1ccc(N(c2ccccc2-c2cccc3oc4ccccc4c23)c2ccc(-c3ccccc3-n3c4ccccc4c4ccccc43)c3oc4ccccc4c23)cc1]

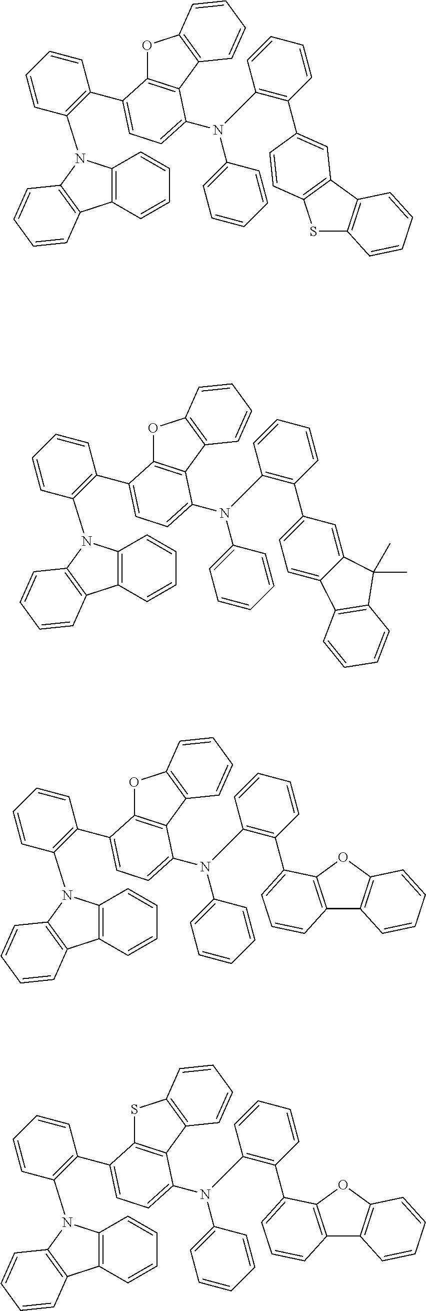 [SMILES: c1ccc(N(c2ccccc2-c2ccc3sc4ccccc4c3c2)c2ccc(-c3ccccc3-n3c4ccccc4c4ccccc43)c3oc4ccccc4c23)cc1 ; CC1(C)c2ccccc2-c2ccc(-c3ccccc3N(c3ccccc3)c3ccc(-c4ccccc4-n4c5ccccc5c5ccccc54)c4oc5ccccc5c34)cc21 ; c1ccc(N(c2ccccc2-c2cccc3c2oc2ccccc23)c2ccc(-c3ccccc3-n3c4ccccc4c4ccccc43)c3oc4ccccc4c23)cc1 ; c1ccc(N(c2ccccc2-c2cccc3c2oc2ccccc23)c2ccc(-c3ccccc3-n3c4ccccc4c4ccccc43)c3sc4ccccc4c23)cc1]

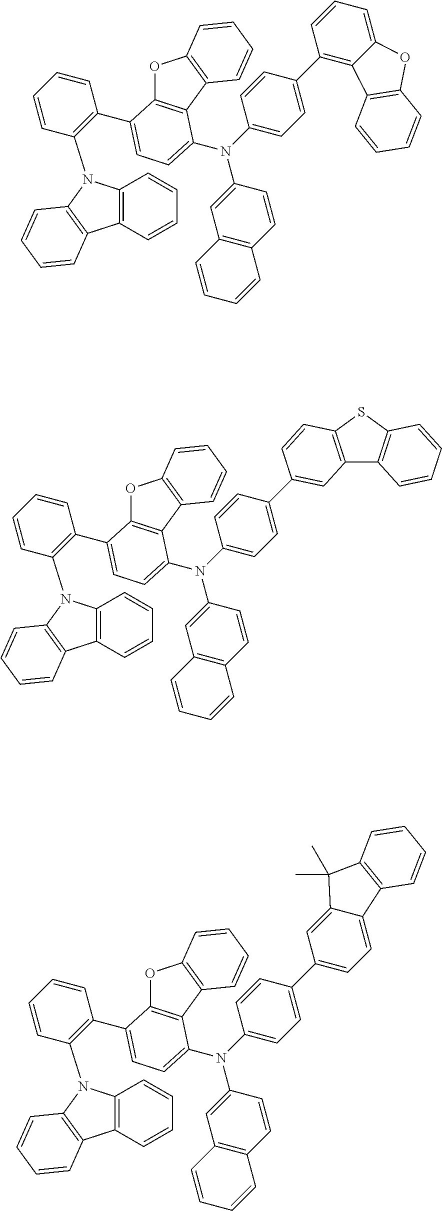 [SMILES: c1ccc(-n2c3ccccc3c3ccccc32)c(-c2ccc(N(c3ccc(-c4cccc5oc6ccccc6c45)cc3)c3ccc4ccccc4c3)c3c2oc2ccccc23)c1 ; c1ccc(-n2c3ccccc3c3ccccc32)c(-c2ccc(N(c3ccc(-c4ccc5sc6ccccc6c5c4)cc3)c3ccc4ccccc4c3)c3c2oc2ccccc23)c1 ; CC1(C)c2ccccc2-c2ccc(-c3ccc(N(c4ccc5ccccc5c4)c4ccc(-c5ccccc5-n5c6ccccc6c6ccccc65)c5oc6ccccc6c45)cc3)cc21]

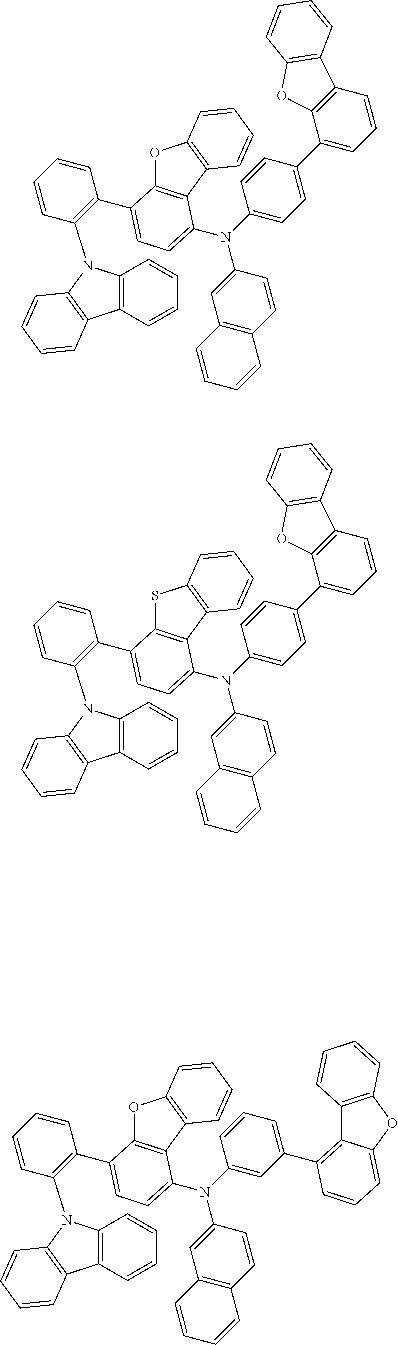 [SMILES: c1ccc(-n2c3ccccc3c3ccccc32)c(-c2ccc(N(c3ccc(-c4cccc5c4oc4ccccc45)cc3)c3ccc4ccccc4c3)c3c2oc2ccccc23)c1 ; c1ccc(-n2c3ccccc3c3ccccc32)c(-c2ccc(N(c3ccc(-c4cccc5c4oc4ccccc45)cc3)c3ccc4ccccc4c3)c3c2sc2ccccc23)c1 ; c1cc(-c2cccc3oc4ccccc4c23)cc(N(c2ccc3ccccc3c2)c2ccc(-c3ccccc3-n3c4ccccc4c4ccccc43)c3oc4ccccc4c23)c1]

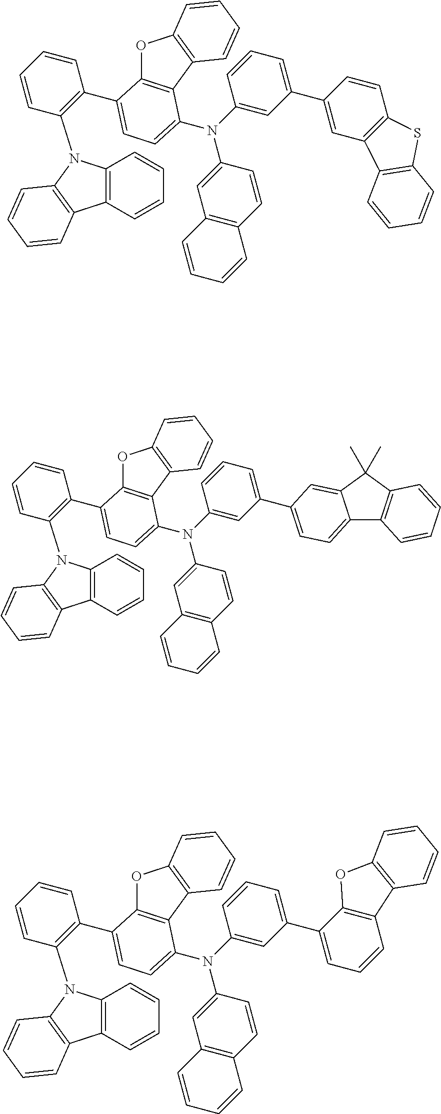 [SMILES: c1cc(-c2ccc3sc4ccccc4c3c2)cc(N(c2ccc3ccccc3c2)c2ccc(-c3ccccc3-n3c4ccccc4c4ccccc43)c3oc4ccccc4c23)c1 ; CC1(C)c2ccccc2-c2ccc(-c3cccc(N(c4ccc5ccccc5c4)c4ccc(-c5ccccc5-n5c6ccccc6c6ccccc65)c5oc6ccccc6c45)c3)cc21 ; c1cc(-c2cccc3c2oc2ccccc23)cc(N(c2ccc3ccccc3c2)c2ccc(-c3ccccc3-n3c4ccccc4c4ccccc43)c3oc4ccccc4c23)c1]

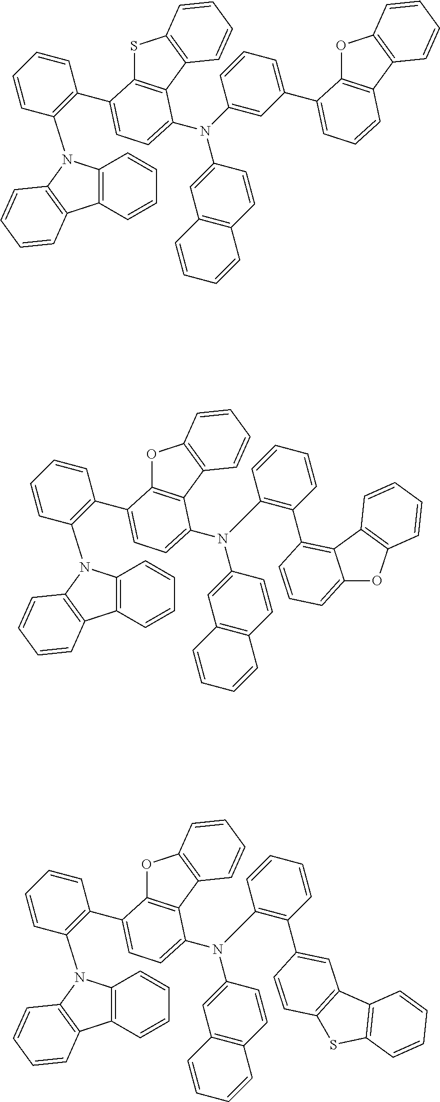 [SMILES: c1cc(-c2cccc3c2oc2ccccc23)cc(N(c2ccc3ccccc3c2)c2ccc(-c3ccccc3-n3c4ccccc4c4ccccc43)c3sc4ccccc4c23)c1 ; c1ccc(N(c2ccc3ccccc3c2)c2ccc(-c3ccccc3-n3c4ccccc4c4ccccc43)c3oc4ccccc4c23)c(-c2cccc3oc4ccccc4c23)c1 ; c1ccc(N(c2ccc3ccccc3c2)c2ccc(-c3ccccc3-n3c4ccccc4c4ccccc43)c3oc4ccccc4c23)c(-c2ccc3sc4ccccc4c3c2)c1]

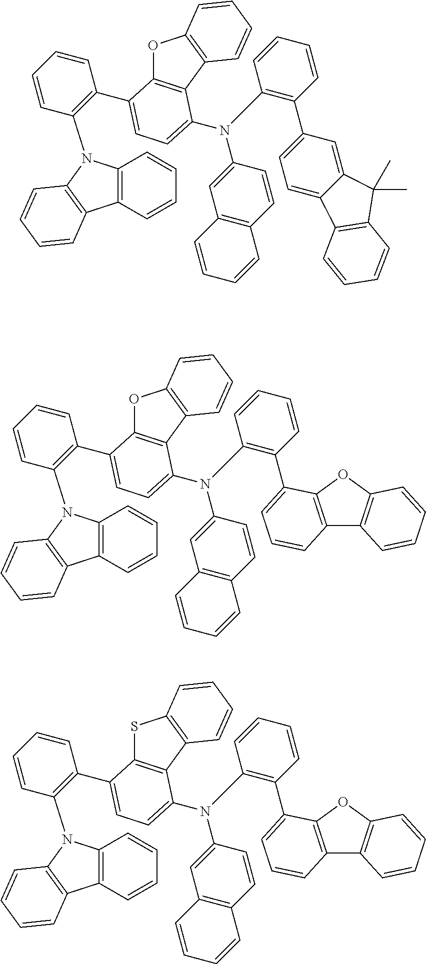

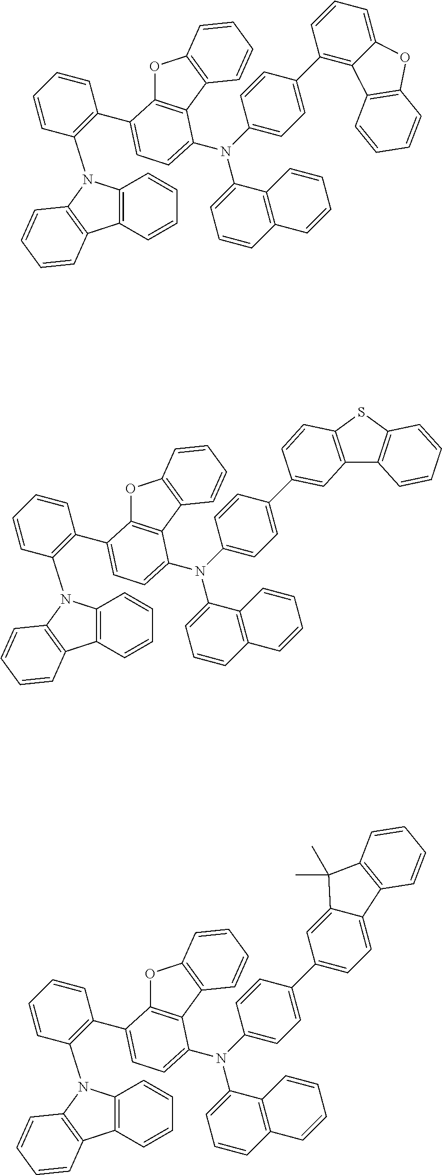 [SMILES: c1ccc(-n2c3ccccc3c3ccccc32)c(-c2ccc(N(c3ccc(-c4cccc5oc6ccccc6c45)cc3)c3cccc4ccccc34)c3c2oc2ccccc23)c1 ; c1ccc(-n2c3ccccc3c3ccccc32)c(-c2ccc(N(c3ccc(-c4ccc5sc6ccccc6c5c4)cc3)c3cccc4ccccc34)c3c2oc2ccccc23)c1 ; CC1(C)c2ccccc2-c2ccc(-c3ccc(N(c4cccc5ccccc45)c4ccc(-c5ccccc5-n5c6ccccc6c6ccccc65)c5oc6ccccc6c45)cc3)cc21]

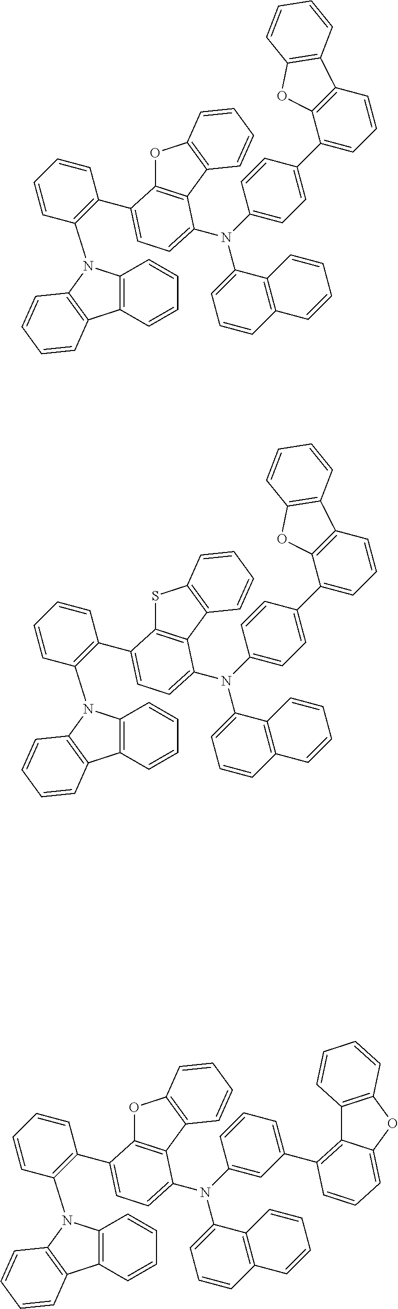 [SMILES: c1ccc(-n2c3ccccc3c3ccccc32)c(-c2ccc(N(c3ccc(-c4cccc5c4oc4ccccc45)cc3)c3cccc4ccccc34)c3c2oc2ccccc23)c1 ; c1ccc(-n2c3ccccc3c3ccccc32)c(-c2ccc(N(c3ccc(-c4cccc5c4oc4ccccc45)cc3)c3cccc4ccccc34)c3c2sc2ccccc23)c1 ; c1cc(-c2cccc3oc4ccccc4c23)cc(N(c2cccc3ccccc23)c2ccc(-c3ccccc3-n3c4ccccc4c4ccccc43)c3oc4ccccc4c23)c1]

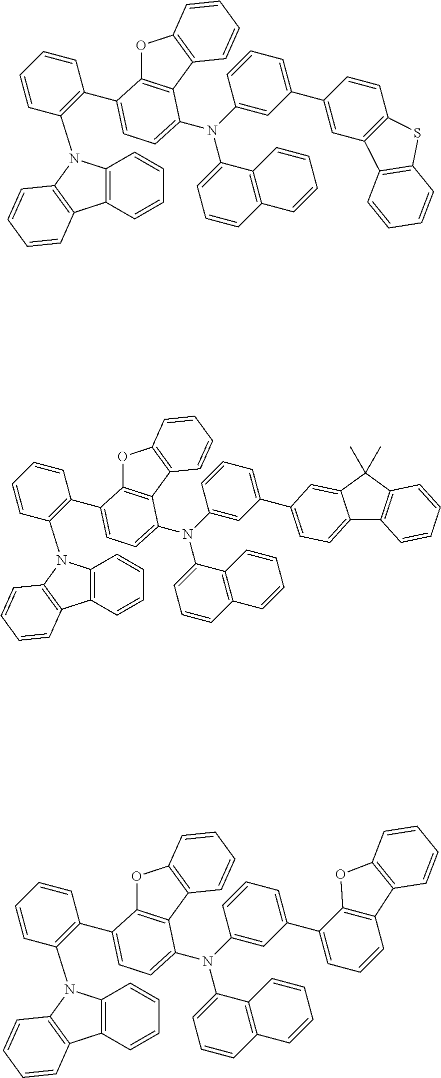 [SMILES: c1cc(-c2ccc3sc4ccccc4c3c2)cc(N(c2cccc3ccccc23)c2ccc(-c3ccccc3-n3c4ccccc4c4ccccc43)c3oc4ccccc4c23)c1 ; CC1(C)c2ccccc2-c2ccc(-c3cccc(N(c4cccc5ccccc45)c4ccc(-c5ccccc5-n5c6ccccc6c6ccccc65)c5oc6ccccc6c45)c3)cc21 ; c1cc(-c2cccc3c2oc2ccccc23)cc(N(c2cccc3ccccc23)c2ccc(-c3ccccc3-n3c4ccccc4c4ccccc43)c3oc4ccccc4c23)c1]

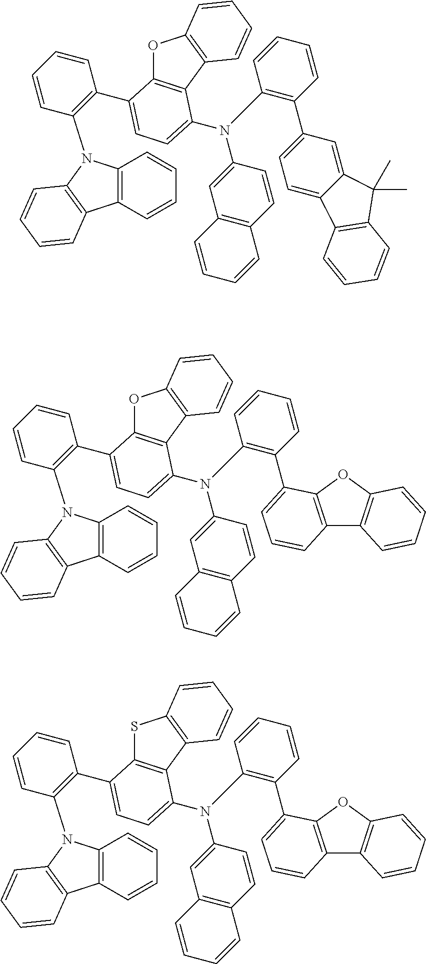

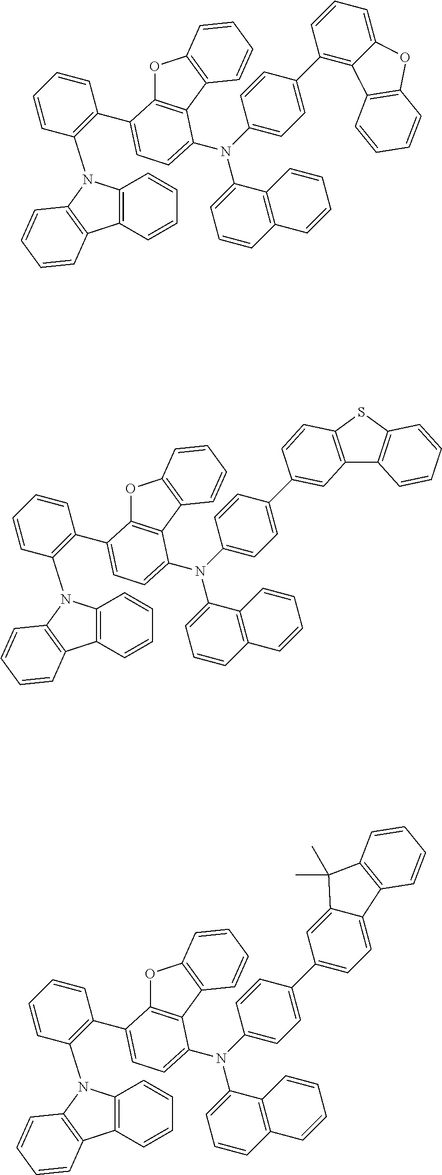 [SMILES: c1ccc(-n2c3ccccc3c3ccccc32)c(-c2ccc(N(c3ccc(-c4cccc5oc6ccccc6c45)cc3)c3cccc4ccccc34)c3c2oc2ccccc23)c1 ; c1ccc(-n2c3ccccc3c3ccccc32)c(-c2ccc(N(c3ccc(-c4ccc5sc6ccccc6c5c4)cc3)c3cccc4ccccc34)c3c2oc2ccccc23)c1 ; CC1(C)c2ccccc2-c2ccc(-c3ccc(N(c4cccc5ccccc45)c4ccc(-c5ccccc5-n5c6ccccc6c6ccccc65)c5oc6ccccc6c45)cc3)cc21]

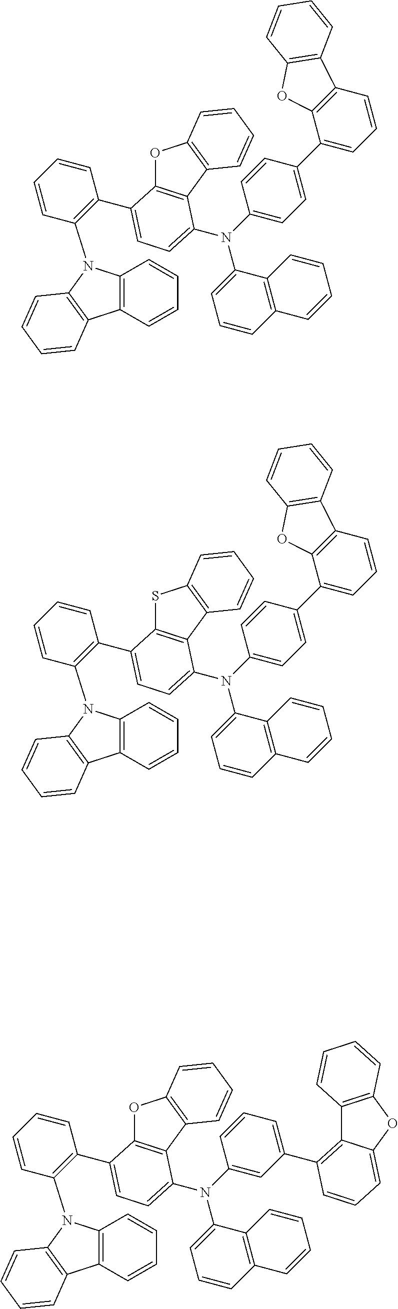 [SMILES: c1ccc(-n2c3ccccc3c3ccccc32)c(-c2ccc(N(c3ccc(-c4cccc5c4oc4ccccc45)cc3)c3cccc4ccccc34)c3c2oc2ccccc23)c1 ; c1ccc(-n2c3ccccc3c3ccccc32)c(-c2ccc(N(c3ccc(-c4cccc5c4oc4ccccc45)cc3)c3cccc4ccccc34)c3c2sc2ccccc23)c1 ; c1cc(-c2cccc3oc4ccccc4c23)cc(N(c2cccc3ccccc23)c2ccc(-c3ccccc3-n3c4ccccc4c4ccccc43)c3oc4ccccc4c23)c1]

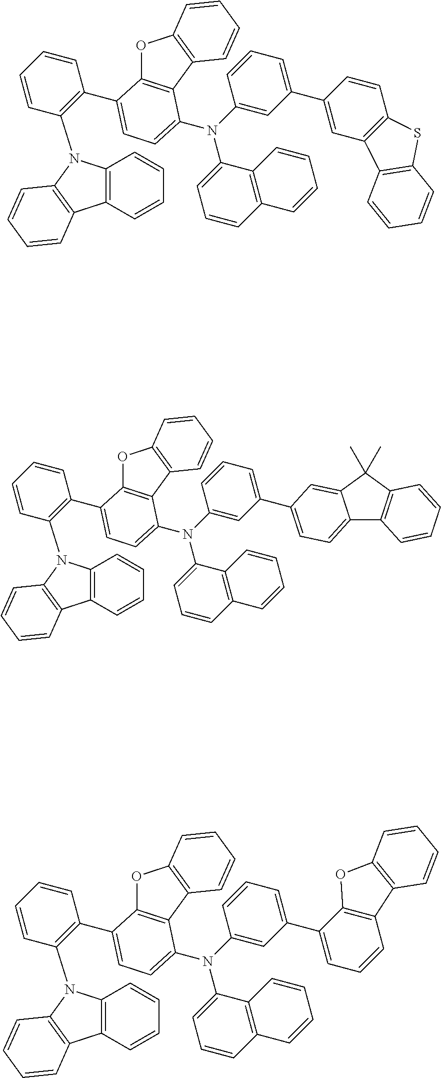 [SMILES: c1cc(-c2ccc3sc4ccccc4c3c2)cc(N(c2cccc3ccccc23)c2ccc(-c3ccccc3-n3c4ccccc4c4ccccc43)c3oc4ccccc4c23)c1 ; CC1(C)c2ccccc2-c2ccc(-c3cccc(N(c4cccc5ccccc45)c4ccc(-c5ccccc5-n5c6ccccc6c6ccccc65)c5oc6ccccc6c45)c3)cc21 ; c1cc(-c2cccc3c2oc2ccccc23)cc(N(c2cccc3ccccc23)c2ccc(-c3ccccc3-n3c4ccccc4c4ccccc43)c3oc4ccccc4c23)c1]

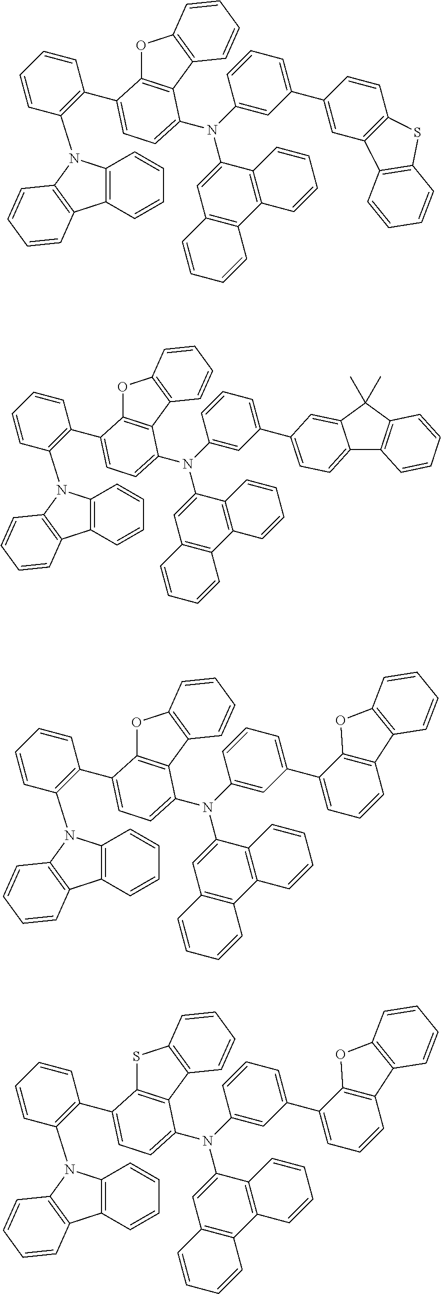 [SMILES: c1cc(-c2ccc3sc4ccccc4c3c2)cc(N(c2cc3ccccc3c3ccccc23)c2ccc(-c3ccccc3-n3c4ccccc4c4ccccc43)c3oc4ccccc4c23)c1 ; CC1(C)c2ccccc2-c2ccc(-c3cccc(N(c4cc5ccccc5c5ccccc45)c4ccc(-c5ccccc5-n5c6ccccc6c6ccccc65)c5oc6ccccc6c45)c3)cc21 ; c1cc(-c2cccc3c2oc2ccccc23)cc(N(c2cc3ccccc3c3ccccc23)c2ccc(-c3ccccc3-n3c4ccccc4c4ccccc43)c3oc4ccccc4c23)c1 ; c1cc(-c2cccc3c2oc2ccccc23)cc(N(c2cc3ccccc3c3ccccc23)c2ccc(-c3ccccc3-n3c4ccccc4c4ccccc43)c3sc4ccccc4c23)c1]

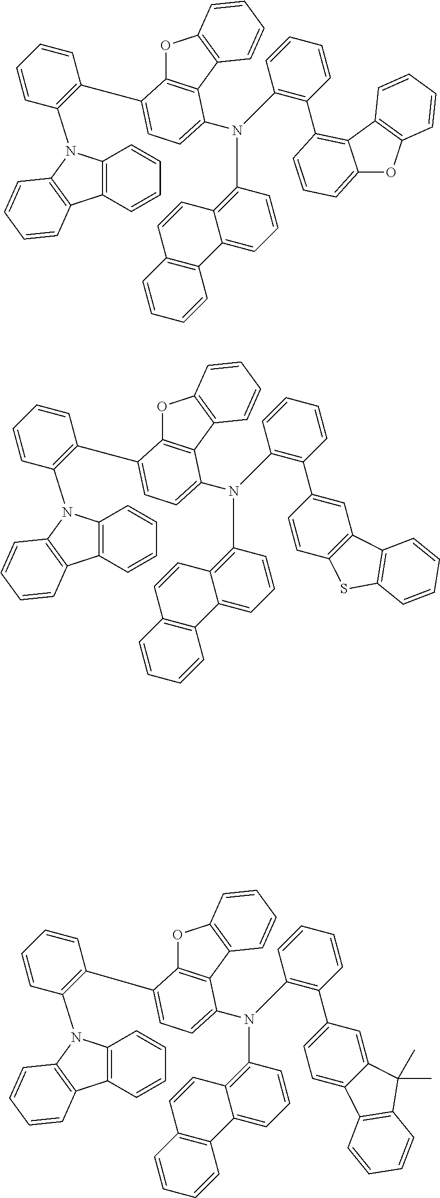 [SMILES: c1ccc(N(c2cccc3c2ccc2ccccc23)c2ccc(-c3ccccc3-n3c4ccccc4c4ccccc43)c3oc4ccccc4c23)c(-c2cccc3oc4ccccc4c23)c1 ; c1ccc(N(c2cccc3c2ccc2ccccc23)c2ccc(-c3ccccc3-n3c4ccccc4c4ccccc43)c3oc4ccccc4c23)c(-c2ccc3sc4ccccc4c3c2)c1 ; CC1(C)c2ccccc2-c2ccc(-c3ccccc3N(c3cccc4c3ccc3ccccc34)c3ccc(-c4ccccc4-n4c5ccccc5c5ccccc54)c4oc5ccccc5c34)cc21]

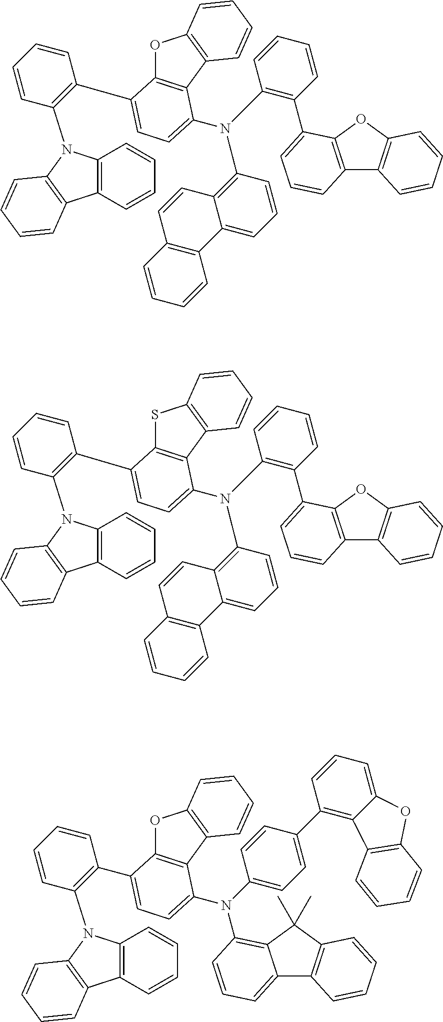 [SMILES: c1ccc(N(c2cccc3c2ccc2ccccc23)c2ccc(-c3ccccc3-n3c4ccccc4c4ccccc43)c3oc4ccccc4c23)c(-c2cccc3c2oc2ccccc23)c1 ; c1ccc(N(c2cccc3c2ccc2ccccc23)c2ccc(-c3ccccc3-n3c4ccccc4c4ccccc43)c3sc4ccccc4c23)c(-c2cccc3c2oc2ccccc23)c1 ; CC1(C)c2ccccc2-c2cccc(N(c3ccc(-c4cccc5oc6ccccc6c45)cc3)c3ccc(-c4ccccc4-n4c5ccccc5c5ccccc54)c4oc5ccccc5c34)c21]

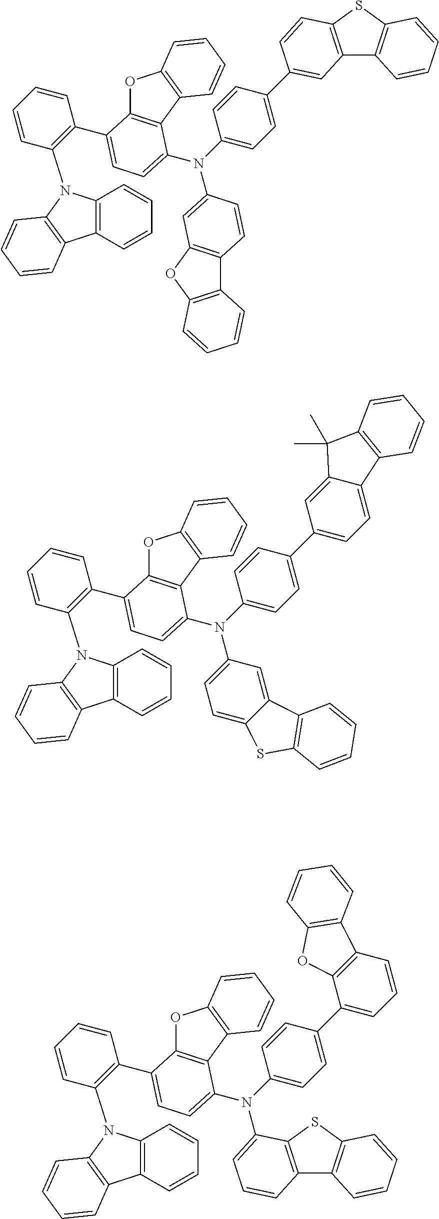 [SMILES: c1ccc(-n2c3ccccc3c3ccccc32)c(-c2ccc(N(c3ccc(-c4ccc5sc6ccccc6c5c4)cc3)c3ccc4c(c3)oc3ccccc34)c3c2oc2ccccc23)c1 ; CC1(C)c2ccccc2-c2ccc(-c3ccc(N(c4ccc5sc6ccccc6c5c4)c4ccc(-c5ccccc5-n5c6ccccc6c6ccccc65)c5oc6ccccc6c45)cc3)cc21 ; c1ccc(-n2c3ccccc3c3ccccc32)c(-c2ccc(N(c3ccc(-c4cccc5c4oc4ccccc45)cc3)c3cccc4c3sc3ccccc34)c3c2oc2ccccc23)c1]

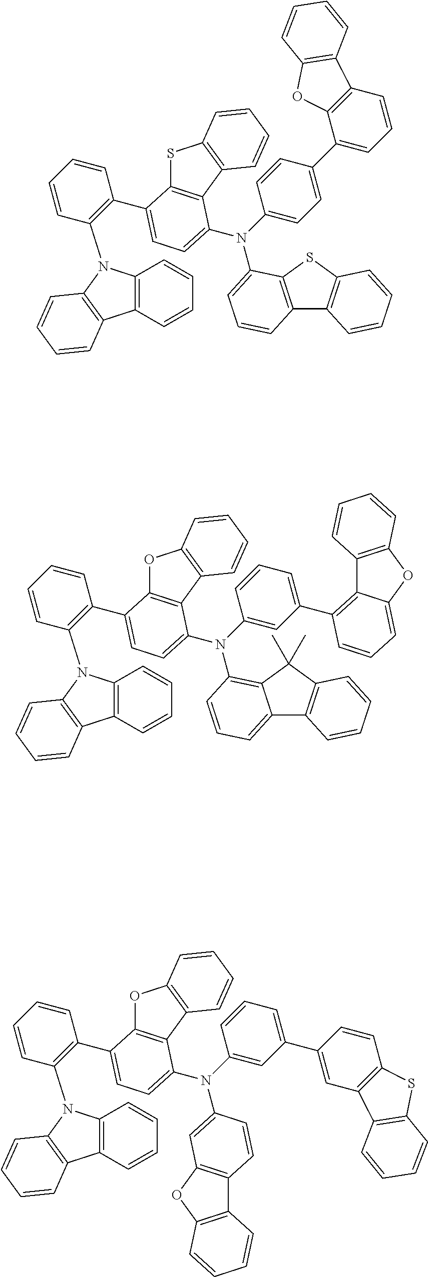 [SMILES: c1ccc(-n2c3ccccc3c3ccccc32)c(-c2ccc(N(c3ccc(-c4cccc5c4oc4ccccc45)cc3)c3cccc4c3sc3ccccc34)c3c2sc2ccccc23)c1 ; CC1(C)c2ccccc2-c2cccc(N(c3cccc(-c4cccc5oc6ccccc6c45)c3)c3ccc(-c4ccccc4-n4c5ccccc5c5ccccc54)c4oc5ccccc5c34)c21 ; c1cc(-c2ccc3sc4ccccc4c3c2)cc(N(c2ccc3c(c2)oc2ccccc23)c2ccc(-c3ccccc3-n3c4ccccc4c4ccccc43)c3oc4ccccc4c23)c1]

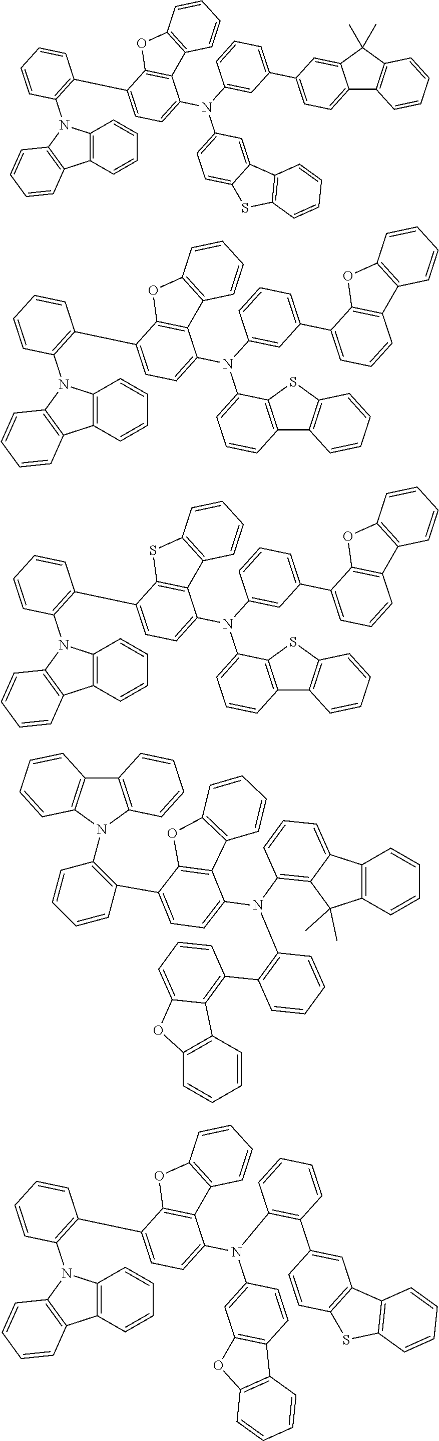

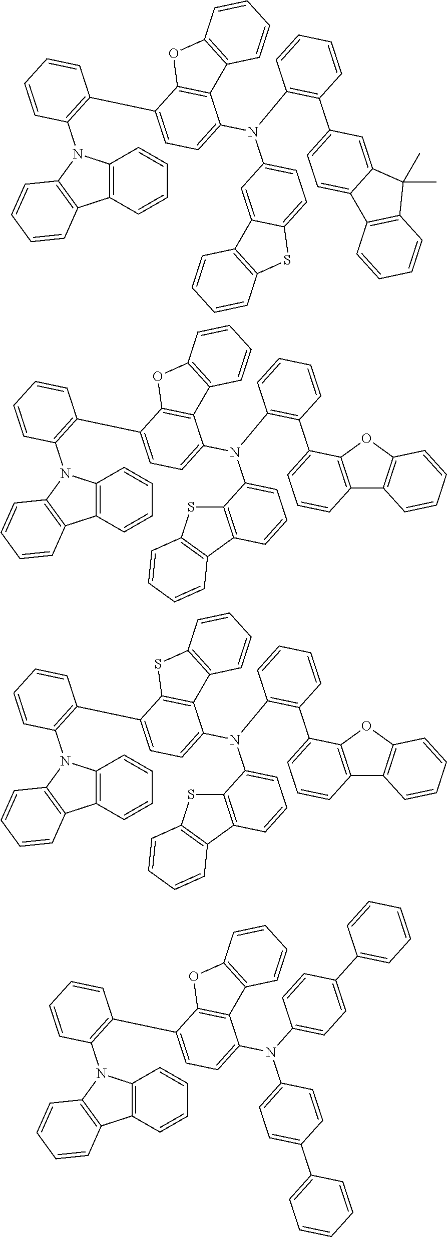

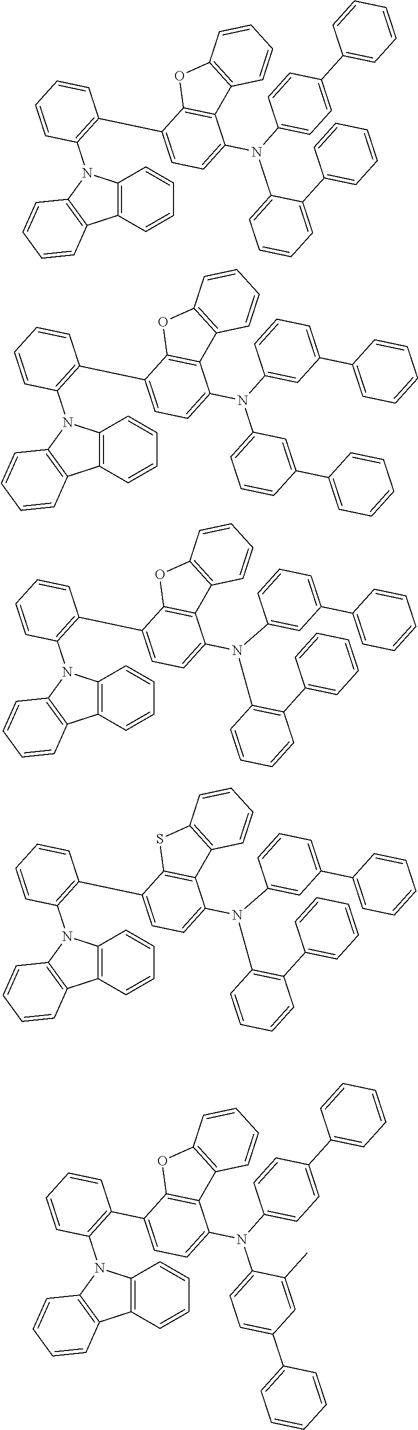

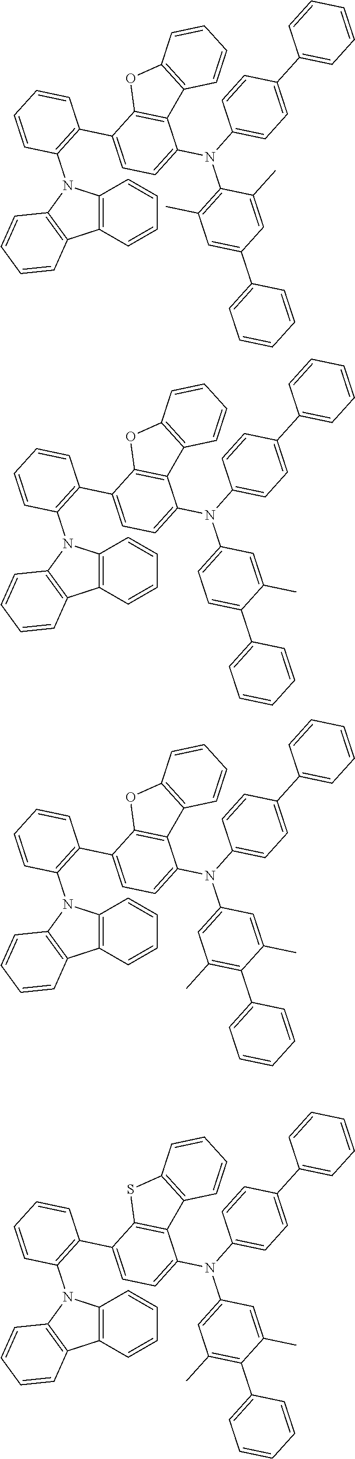

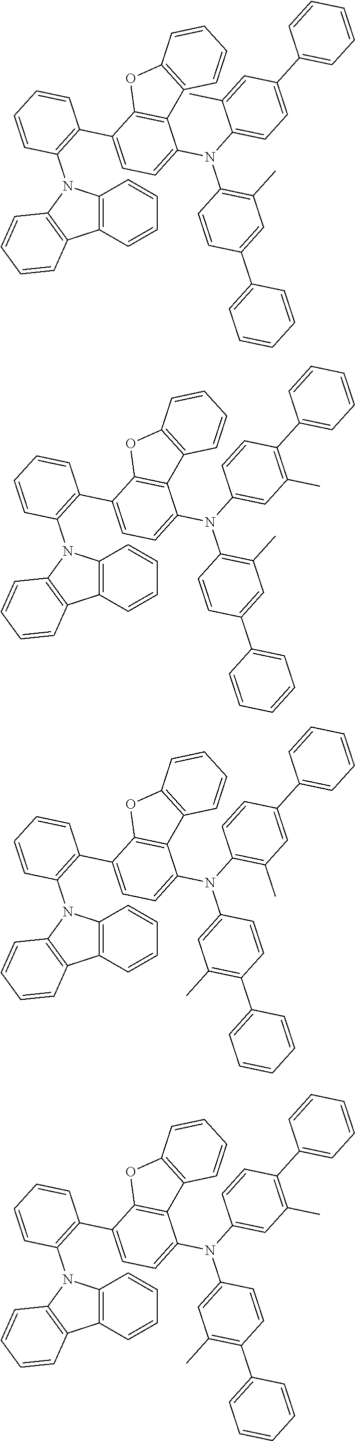

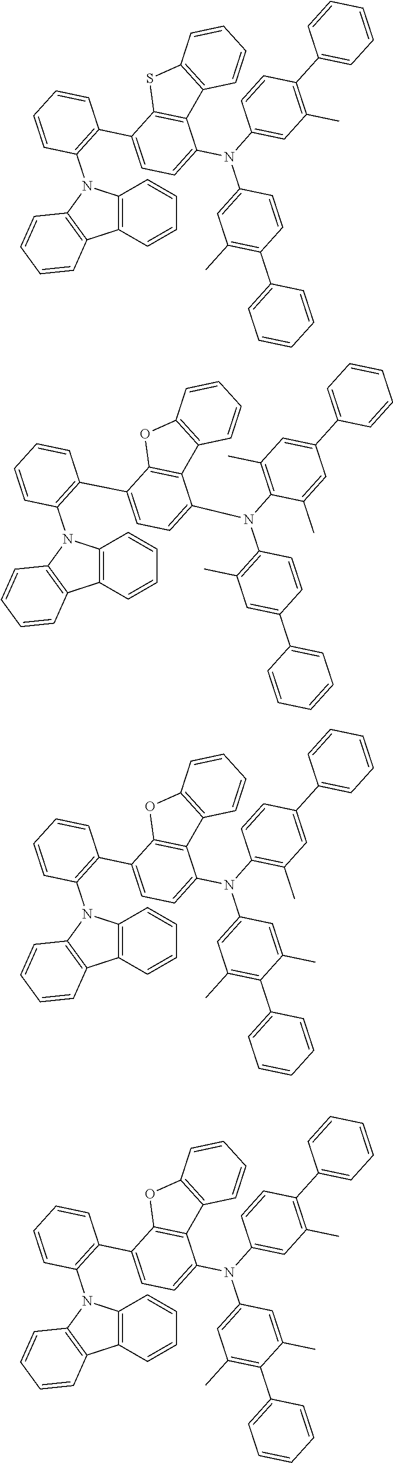

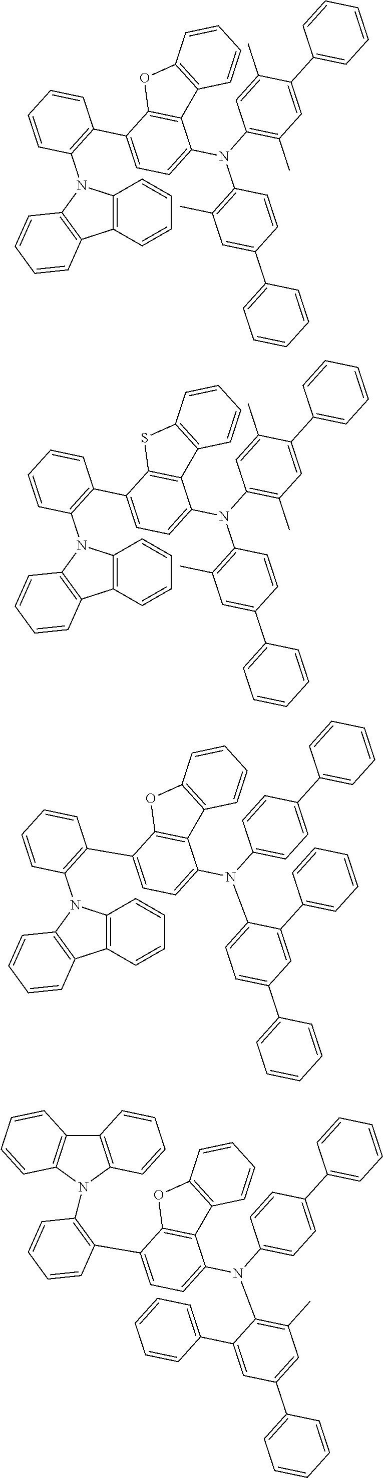

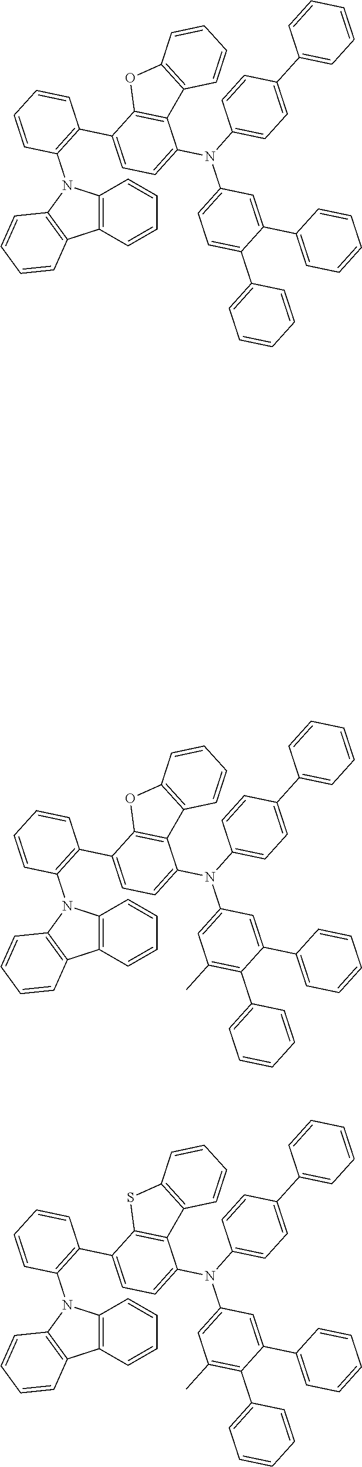 [SMILES: c1ccc(-c2ccc(N(c3ccc(-c4ccccc4)c(-c4ccccc4)c3)c3ccc(-c4ccccc4-n4c5ccccc5c5ccccc54)c4oc5ccccc5c34)cc2)cc1 ; Cc1cc(N(c2ccc(-c3ccccc3)cc2)c2ccc(-c3ccccc3-n3c4ccccc4c4ccccc43)c3oc4ccccc4c23)cc(-c2ccccc2)c1-c1ccccc1 ; Cc1cc(N(c2ccc(-c3ccccc3)cc2)c2ccc(-c3ccccc3-n3c4ccccc4c4ccccc43)c3sc4ccccc4c23)cc(-c2ccccc2)c1-c1ccccc1]

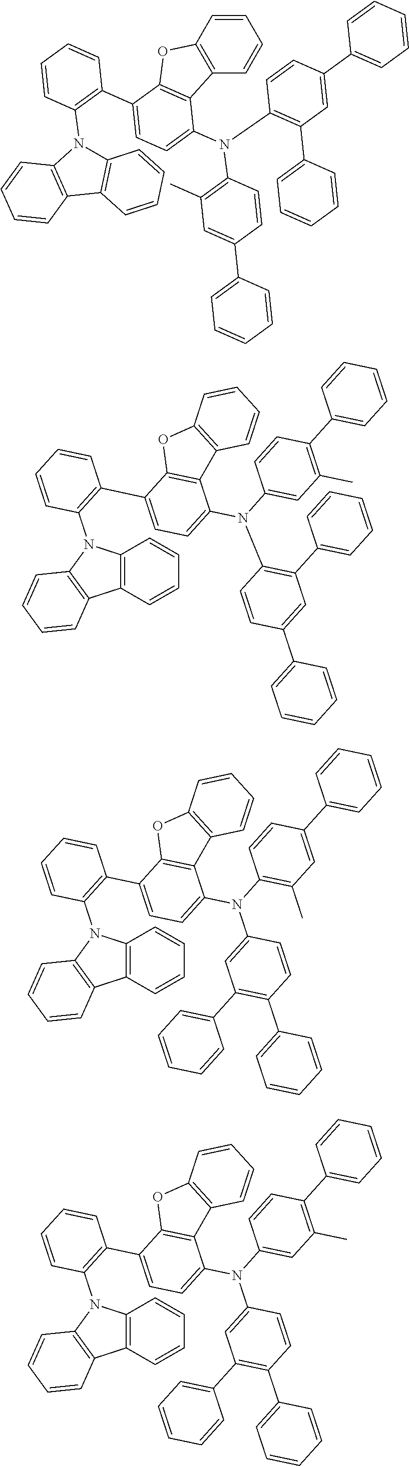

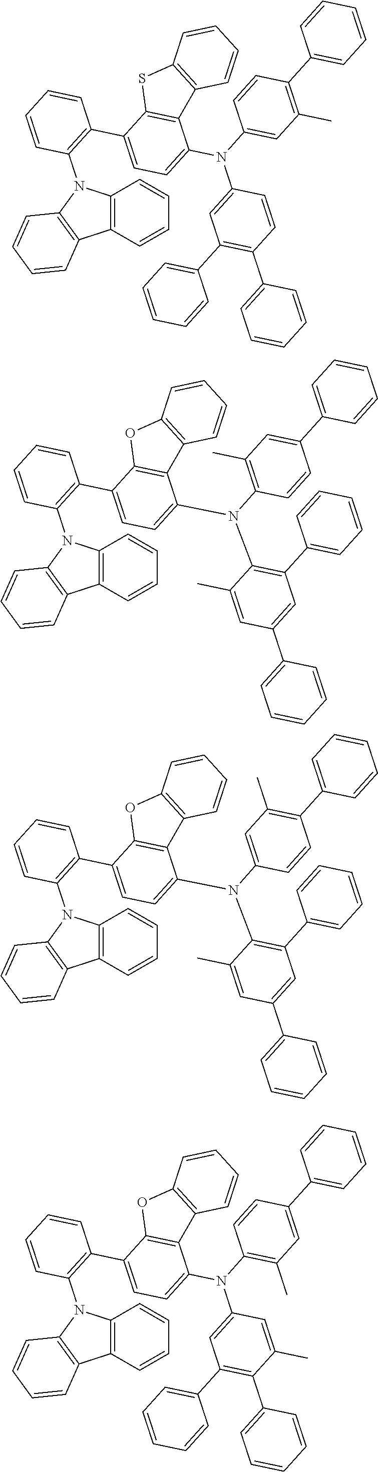

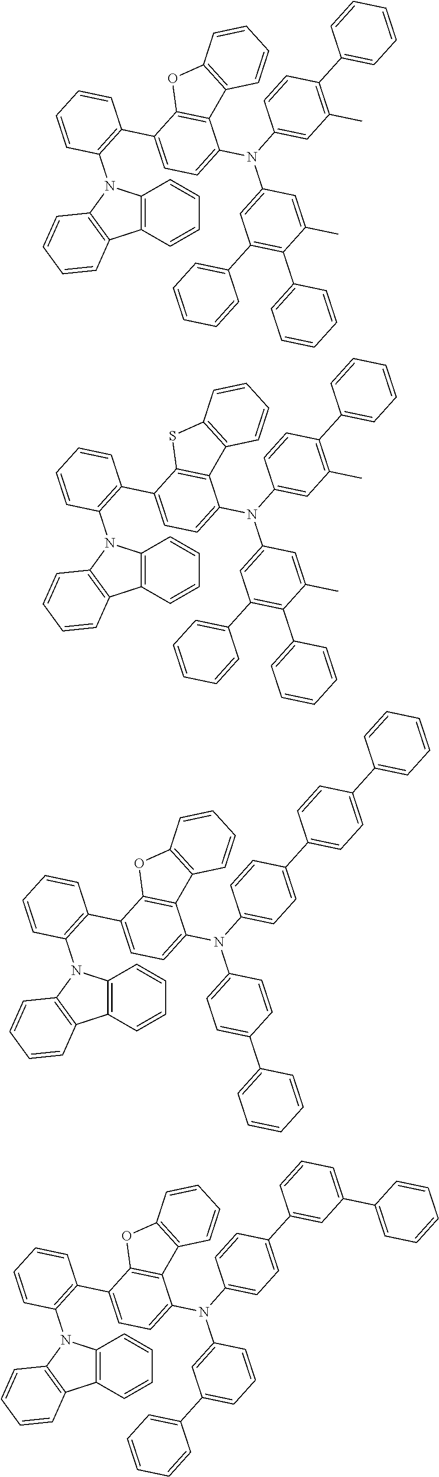

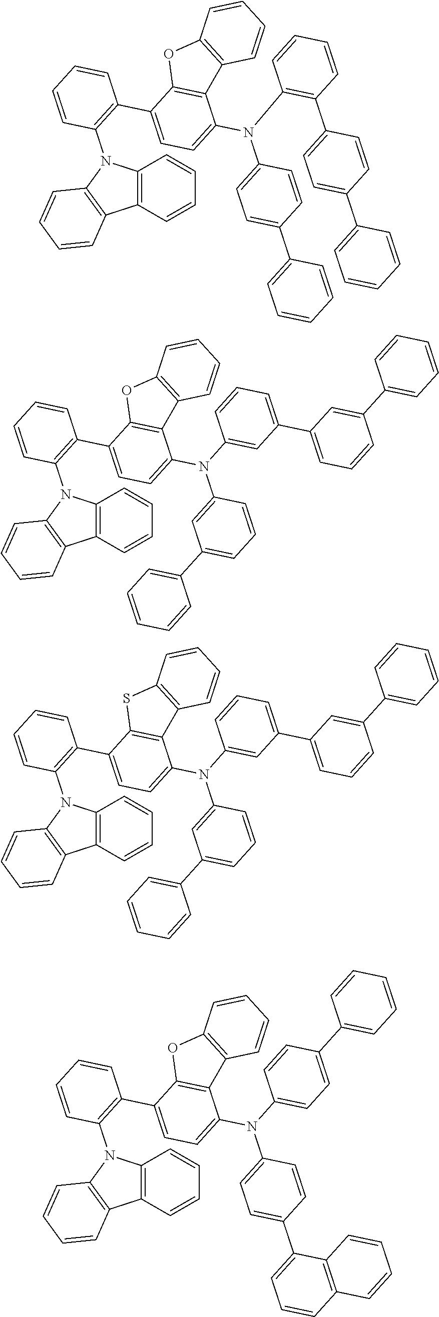

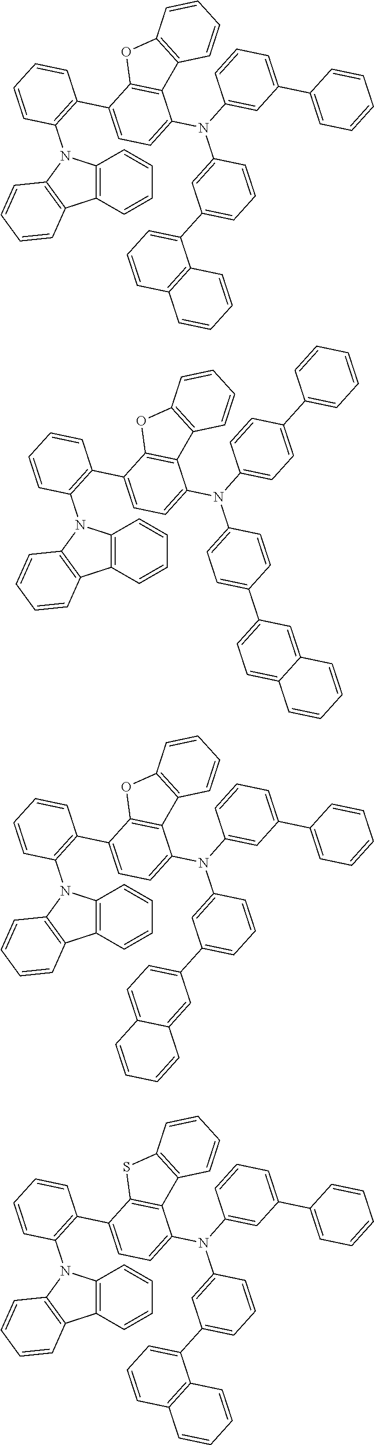

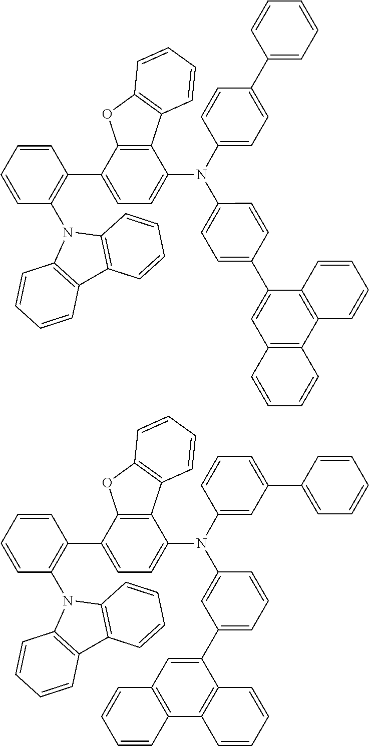 [SMILES: c1ccc(-c2ccc(N(c3ccc(-c4cc5ccccc5c5ccccc45)cc3)c3ccc(-c4ccccc4-n4c5ccccc5c5ccccc54)c4oc5ccccc5c34)cc2)cc1 ; c1ccc(-c2cccc(N(c3cccc(-c4cc5ccccc5c5ccccc45)c3)c3ccc(-c4ccccc4-n4c5ccccc5c5ccccc54)c4oc5ccccc5c34)c2)cc1]

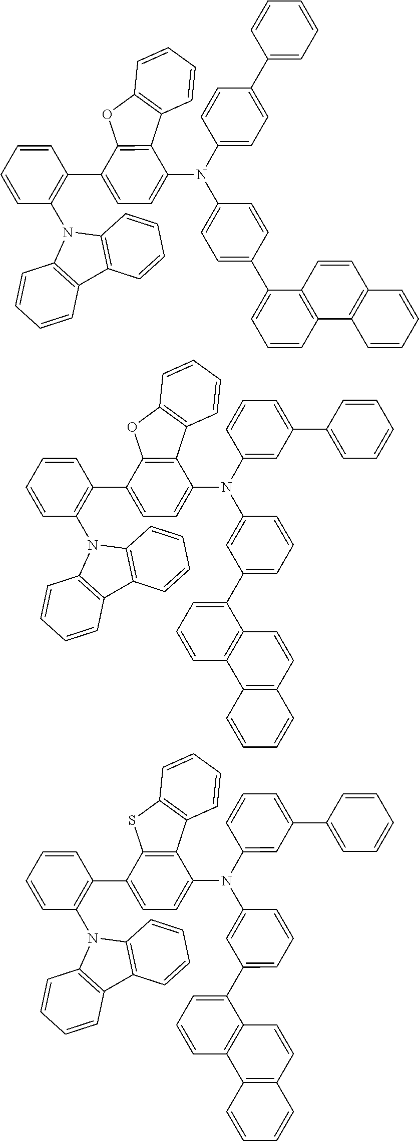

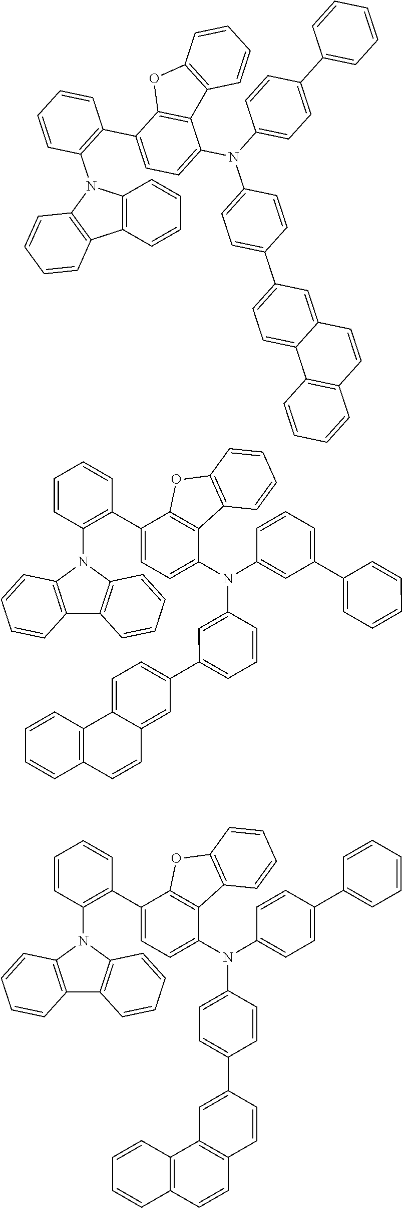

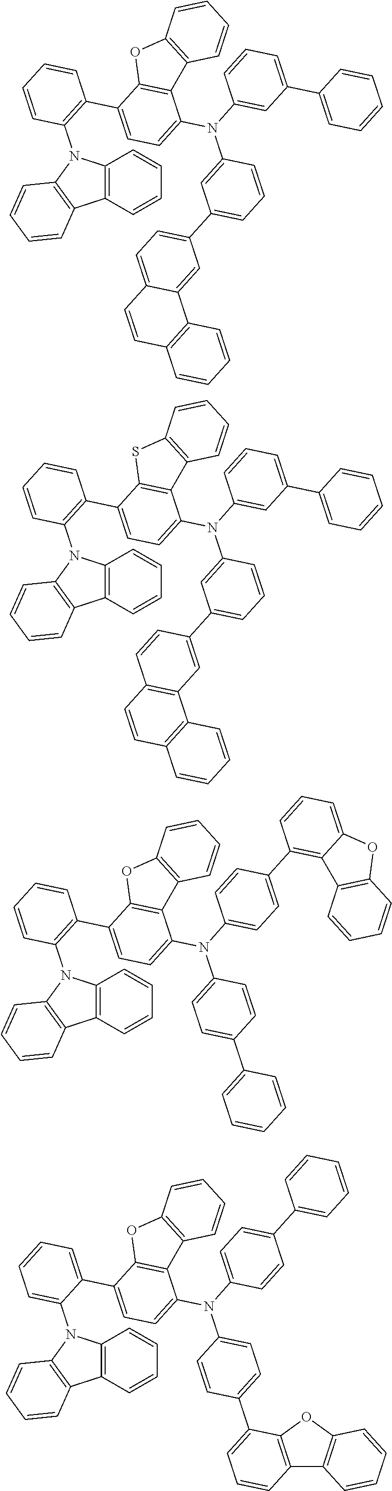

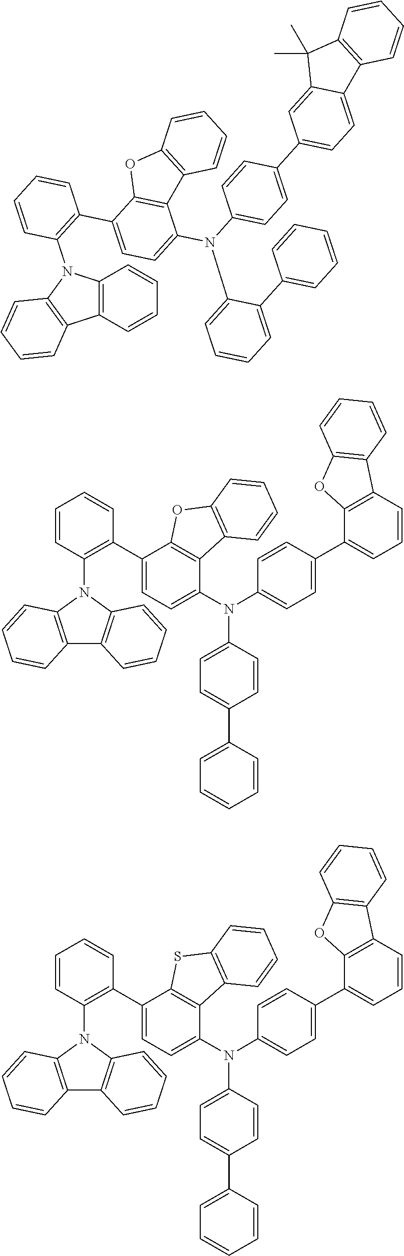 [SMILES: CC1(C)c2ccccc2-c2ccc(-c3ccc(N(c4ccccc4-c4ccccc4)c4ccc(-c5ccccc5-n5c6ccccc6c6ccccc65)c5oc6ccccc6c45)cc3)cc21 ; c1ccc(-c2ccc(N(c3ccc(-c4cccc5c4oc4ccccc45)cc3)c3ccc(-c4ccccc4-n4c5ccccc5c5ccccc54)c4oc5ccccc5c34)cc2)cc1 ; c1ccc(-c2ccc(N(c3ccc(-c4cccc5c4oc4ccccc45)cc3)c3ccc(-c4ccccc4-n4c5ccccc5c5ccccc54)c4sc5ccccc5c34)cc2)cc1]

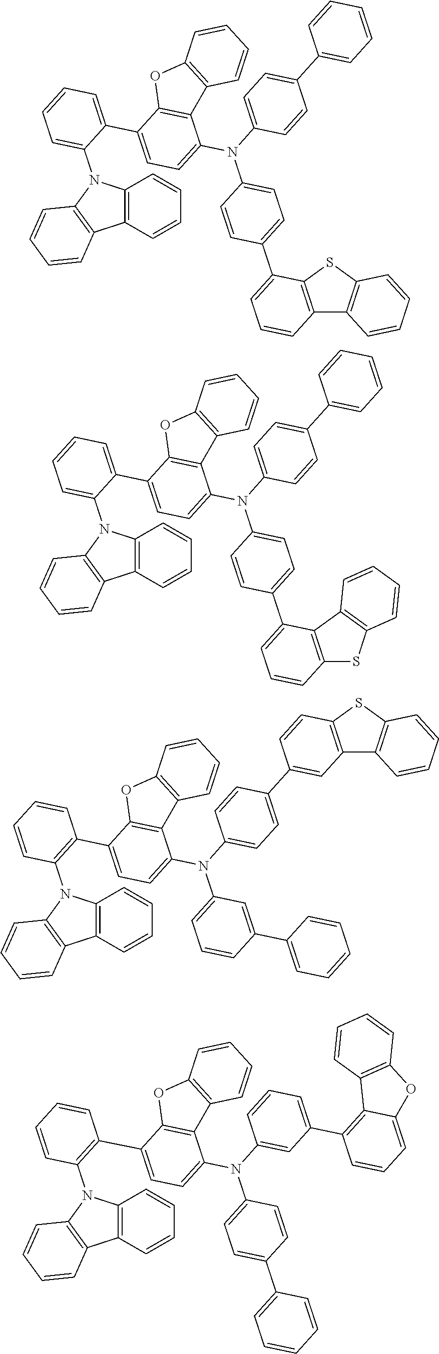

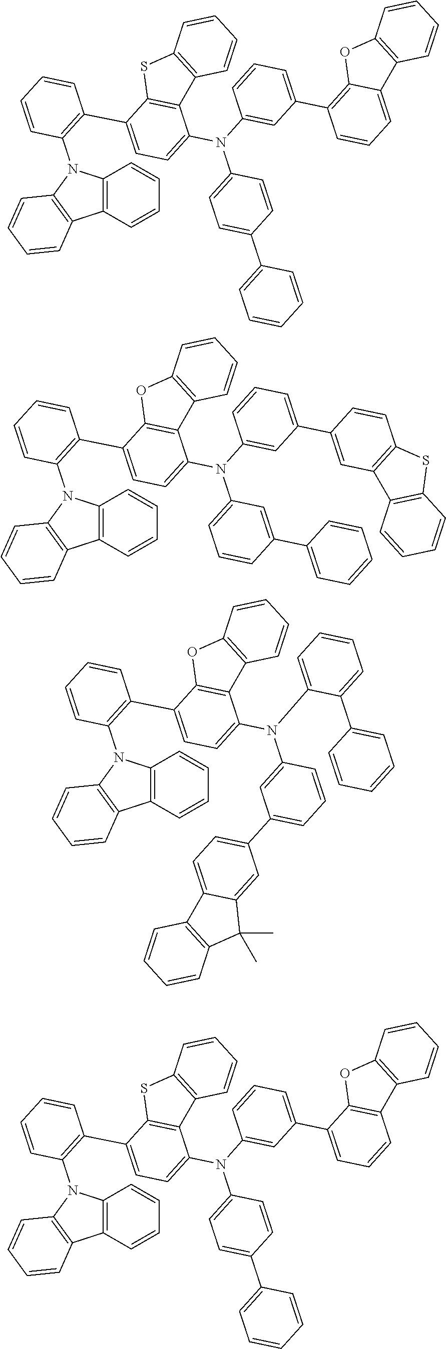

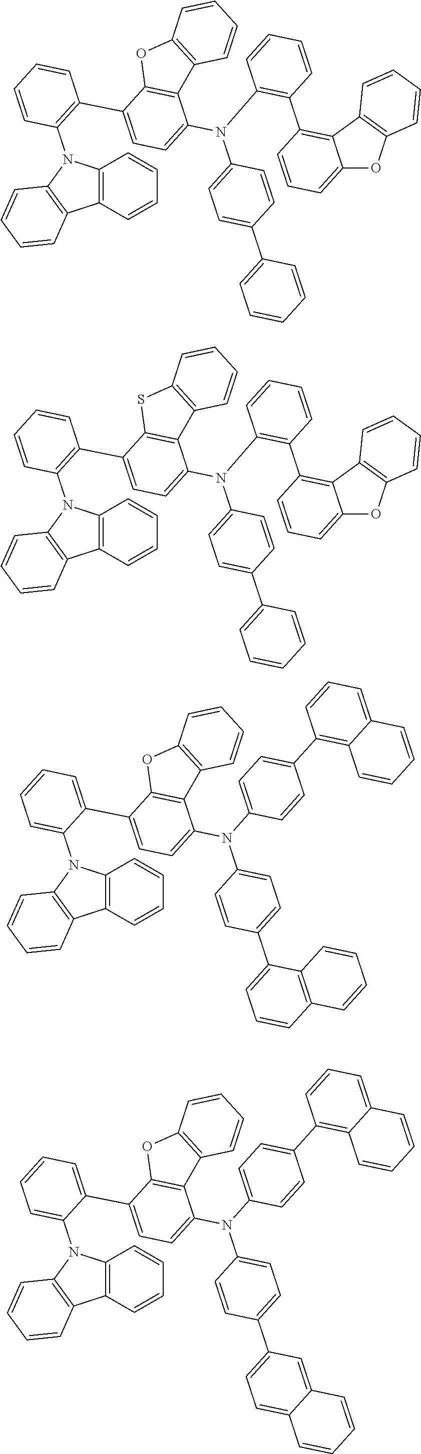

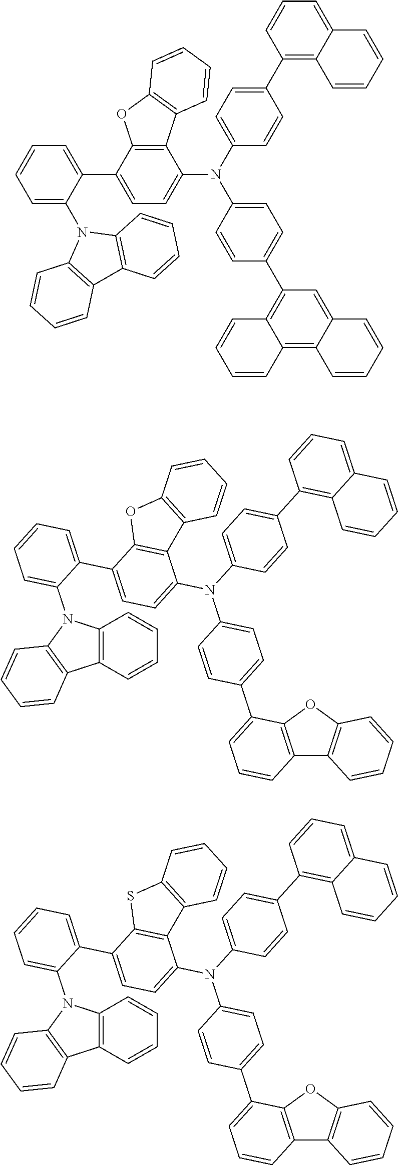

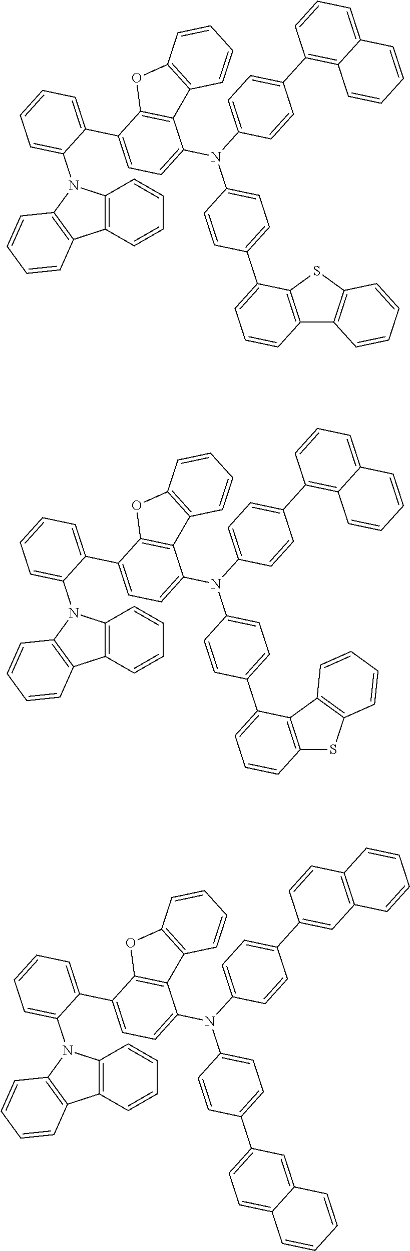 [SMILES: c1ccc(-n2c3ccccc3c3ccccc32)c(-c2ccc(N(c3ccc(-c4cccc5ccccc45)cc3)c3ccc(-c4cccc5c4sc4ccccc45)cc3)c3c2oc2ccccc23)c1 ; c1ccc(-n2c3ccccc3c3ccccc32)c(-c2ccc(N(c3ccc(-c4cccc5ccccc45)cc3)c3ccc(-c4cccc5sc6ccccc6c45)cc3)c3c2oc2ccccc23)c1 ; c1ccc(-n2c3ccccc3c3ccccc32)c(-c2ccc(N(c3ccc(-c4ccc5ccccc5c4)cc3)c3ccc(-c4ccc5ccccc5c4)cc3)c3c2oc2ccccc23)c1]

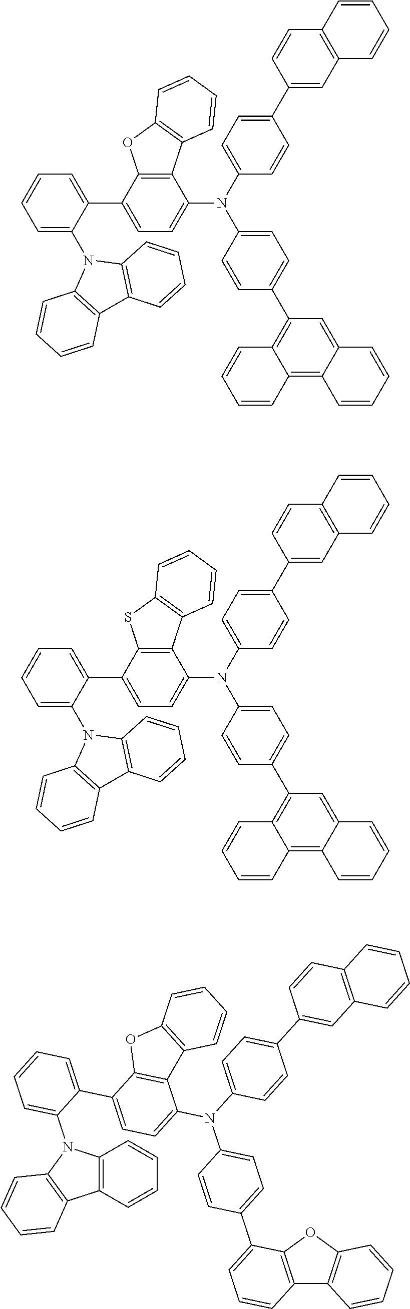 [SMILES: c1ccc(-n2c3ccccc3c3ccccc32)c(-c2ccc(N(c3ccc(-c4ccc5ccccc5c4)cc3)c3ccc(-c4cc5ccccc5c5ccccc45)cc3)c3c2oc2ccccc23)c1 ; c1ccc(-n2c3ccccc3c3ccccc32)c(-c2ccc(N(c3ccc(-c4ccc5ccccc5c4)cc3)c3ccc(-c4cc5ccccc5c5ccccc45)cc3)c3c2sc2ccccc23)c1 ; c1ccc(-n2c3ccccc3c3ccccc32)c(-c2ccc(N(c3ccc(-c4ccc5ccccc5c4)cc3)c3ccc(-c4cccc5c4oc4ccccc45)cc3)c3c2oc2ccccc23)c1]

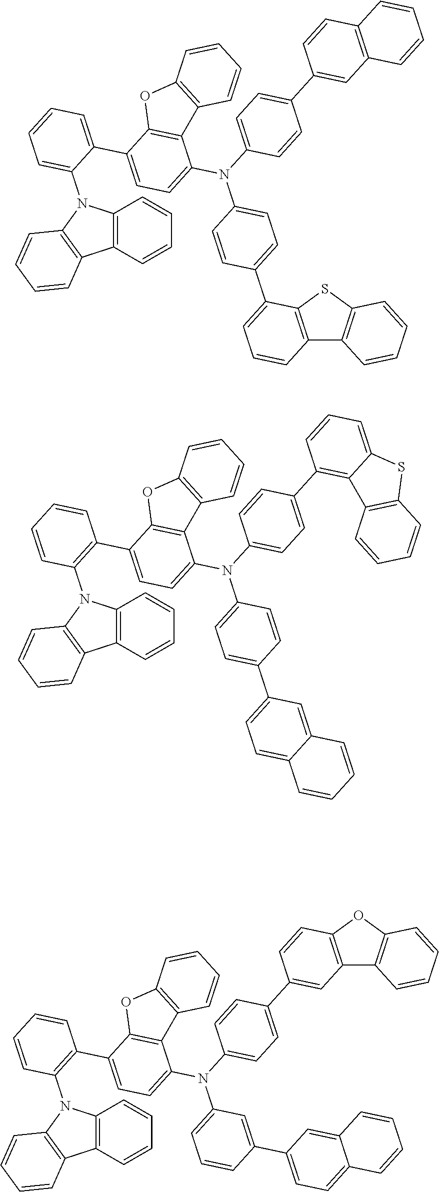 [SMILES: c1ccc(-n2c3ccccc3c3ccccc32)c(-c2ccc(N(c3ccc(-c4ccc5ccccc5c4)cc3)c3ccc(-c4cccc5c4sc4ccccc45)cc3)c3c2oc2ccccc23)c1 ; c1ccc(-n2c3ccccc3c3ccccc32)c(-c2ccc(N(c3ccc(-c4ccc5ccccc5c4)cc3)c3ccc(-c4cccc5sc6ccccc6c45)cc3)c3c2oc2ccccc23)c1 ; c1cc(-c2ccc3ccccc3c2)cc(N(c2ccc(-c3ccc4oc5ccccc5c4c3)cc2)c2ccc(-c3ccccc3-n3c4ccccc4c4ccccc43)c3oc4ccccc4c23)c1]

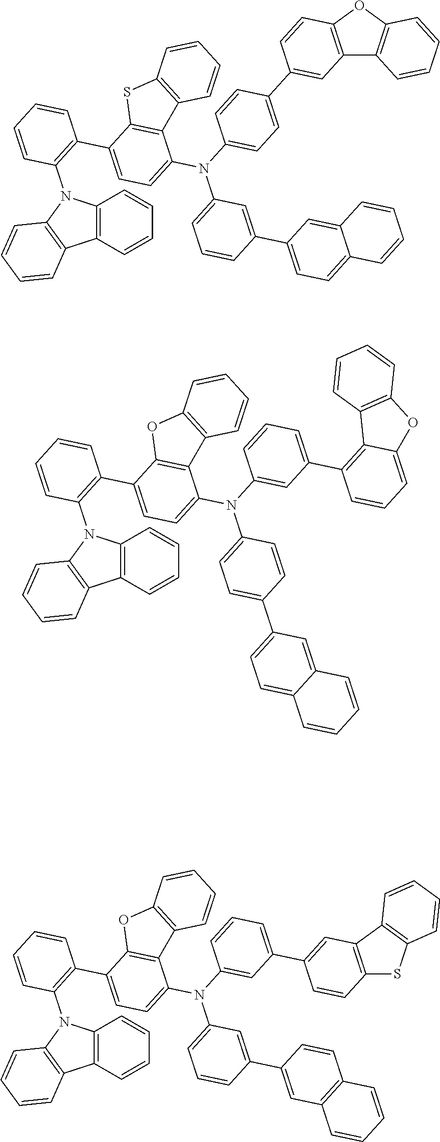 [SMILES: c1cc(-c2ccc3ccccc3c2)cc(N(c2ccc(-c3ccc4oc5ccccc5c4c3)cc2)c2ccc(-c3ccccc3-n3c4ccccc4c4ccccc43)c3sc4ccccc4c23)c1 ; c1cc(-c2cccc3oc4ccccc4c23)cc(N(c2ccc(-c3ccc4ccccc4c3)cc2)c2ccc(-c3ccccc3-n3c4ccccc4c4ccccc43)c3oc4ccccc4c23)c1 ; c1cc(-c2ccc3ccccc3c2)cc(N(c2cccc(-c3ccc4sc5ccccc5c4c3)c2)c2ccc(-c3ccccc3-n3c4ccccc4c4ccccc43)c3oc4ccccc4c23)c1]

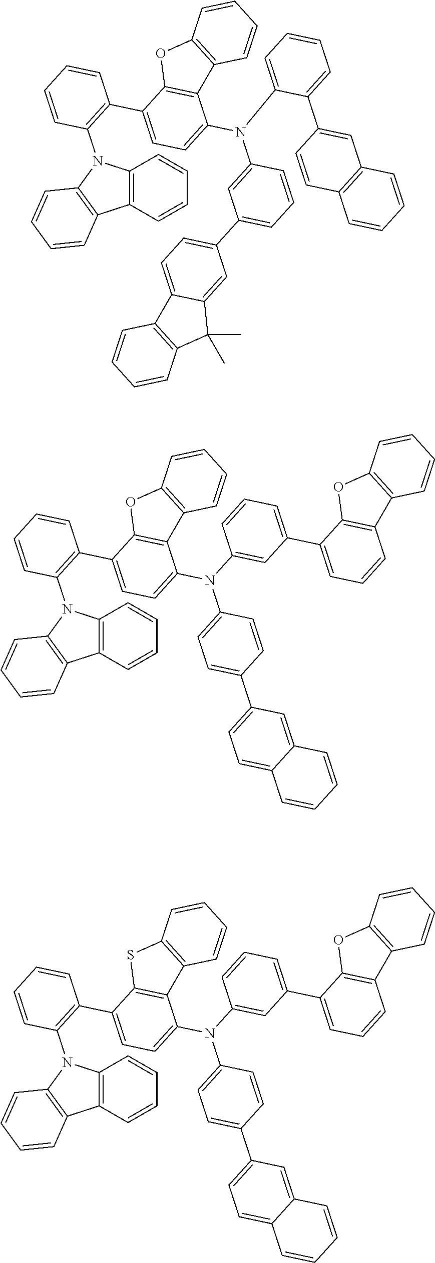 [SMILES: CC1(C)c2ccccc2-c2ccc(-c3cccc(N(c4ccccc4-c4ccc5ccccc5c4)c4ccc(-c5ccccc5-n5c6ccccc6c6ccccc65)c5oc6ccccc6c45)c3)cc21 ; c1cc(-c2cccc3c2oc2ccccc23)cc(N(c2ccc(-c3ccc4ccccc4c3)cc2)c2ccc(-c3ccccc3-n3c4ccccc4c4ccccc43)c3oc4ccccc4c23)c1 ; c1cc(-c2cccc3c2oc2ccccc23)cc(N(c2ccc(-c3ccc4ccccc4c3)cc2)c2ccc(-c3ccccc3-n3c4ccccc4c4ccccc43)c3sc4ccccc4c23)c1]

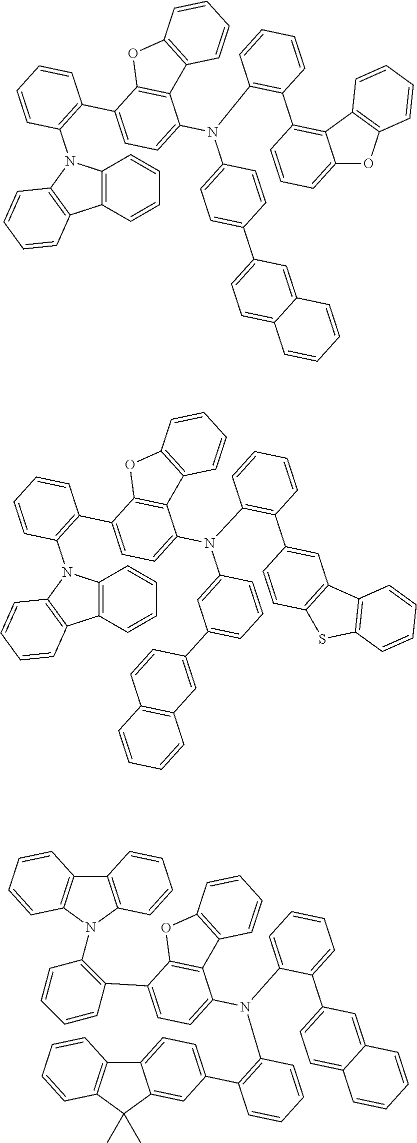 [SMILES: c1ccc(N(c2ccc(-c3ccc4ccccc4c3)cc2)c2ccc(-c3ccccc3-n3c4ccccc4c4ccccc43)c3oc4ccccc4c23)c(-c2cccc3oc4ccccc4c23)c1 ; c1cc(-c2ccc3ccccc3c2)cc(N(c2ccccc2-c2ccc3sc4ccccc4c3c2)c2ccc(-c3ccccc3-n3c4ccccc4c4ccccc43)c3oc4ccccc4c23)c1 ; CC1(C)c2ccccc2-c2ccc(-c3ccccc3N(c3ccccc3-c3ccc4ccccc4c3)c3ccc(-c4ccccc4-n4c5ccccc5c5ccccc54)c4oc5ccccc5c34)cc21]

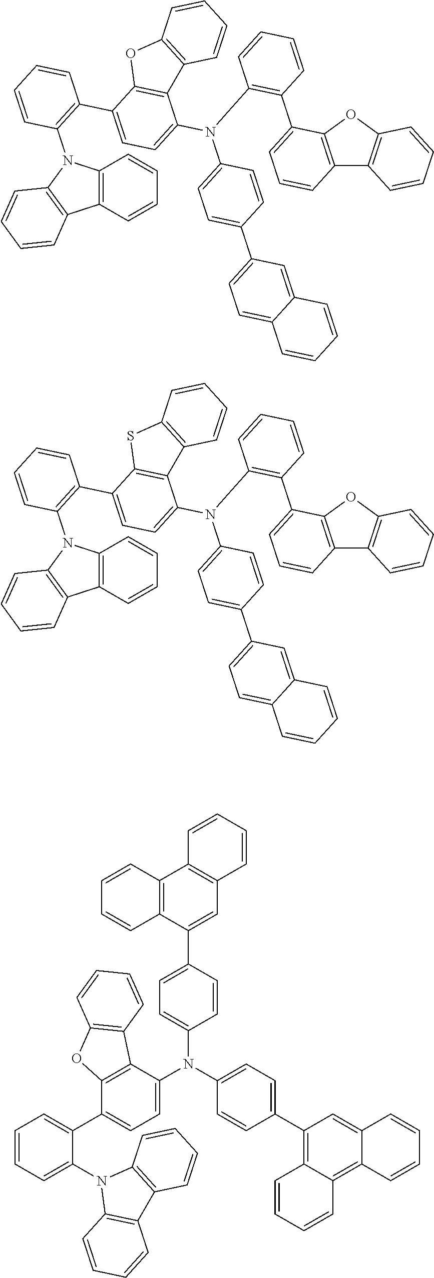 [SMILES: c1ccc(N(c2ccc(-c3ccc4ccccc4c3)cc2)c2ccc(-c3ccccc3-n3c4ccccc4c4ccccc43)c3oc4ccccc4c23)c(-c2cccc3c2oc2ccccc23)c1 ; c1ccc(N(c2ccc(-c3ccc4ccccc4c3)cc2)c2ccc(-c3ccccc3-n3c4ccccc4c4ccccc43)c3sc4ccccc4c23)c(-c2cccc3c2oc2ccccc23)c1 ; c1ccc(-n2c3ccccc3c3ccccc32)c(-c2ccc(N(c3ccc(-c4cc5ccccc5c5ccccc45)cc3)c3ccc(-c4cc5ccccc5c5ccccc45)cc3)c3c2oc2ccccc23)c1]

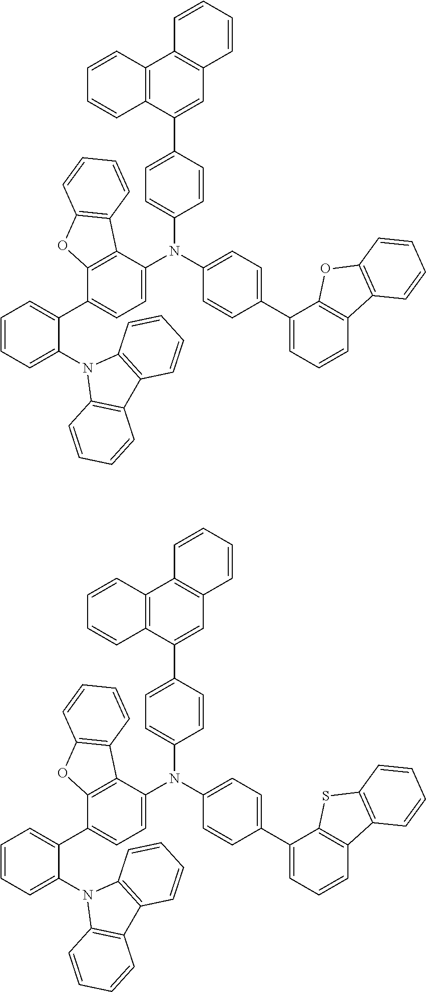 [SMILES: c1ccc(-n2c3ccccc3c3ccccc32)c(-c2ccc(N(c3ccc(-c4cc5ccccc5c5ccccc45)cc3)c3ccc(-c4cccc5c4oc4ccccc45)cc3)c3c2oc2ccccc23)c1 ; c1ccc(-n2c3ccccc3c3ccccc32)c(-c2ccc(N(c3ccc(-c4cc5ccccc5c5ccccc45)cc3)c3ccc(-c4cccc5c4sc4ccccc45)cc3)c3c2oc2ccccc23)c1]

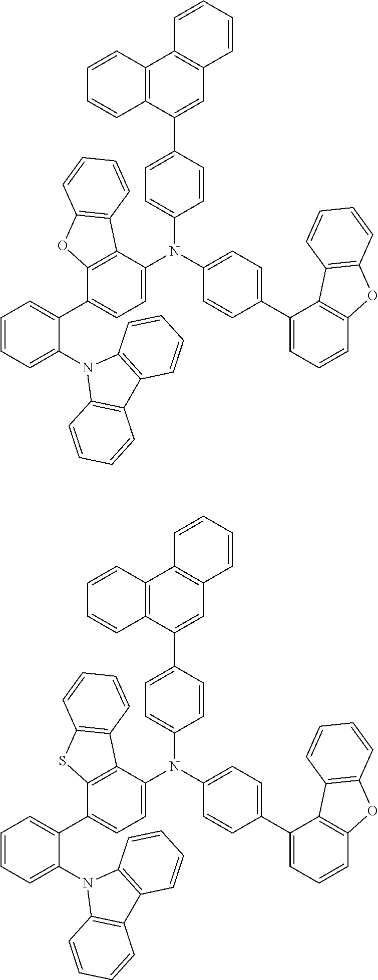 [SMILES: c1ccc(-n2c3ccccc3c3ccccc32)c(-c2ccc(N(c3ccc(-c4cc5ccccc5c5ccccc45)cc3)c3ccc(-c4cccc5oc6ccccc6c45)cc3)c3c2oc2ccccc23)c1 ; c1ccc(-n2c3ccccc3c3ccccc32)c(-c2ccc(N(c3ccc(-c4cc5ccccc5c5ccccc45)cc3)c3ccc(-c4cccc5oc6ccccc6c45)cc3)c3c2sc2ccccc23)c1]

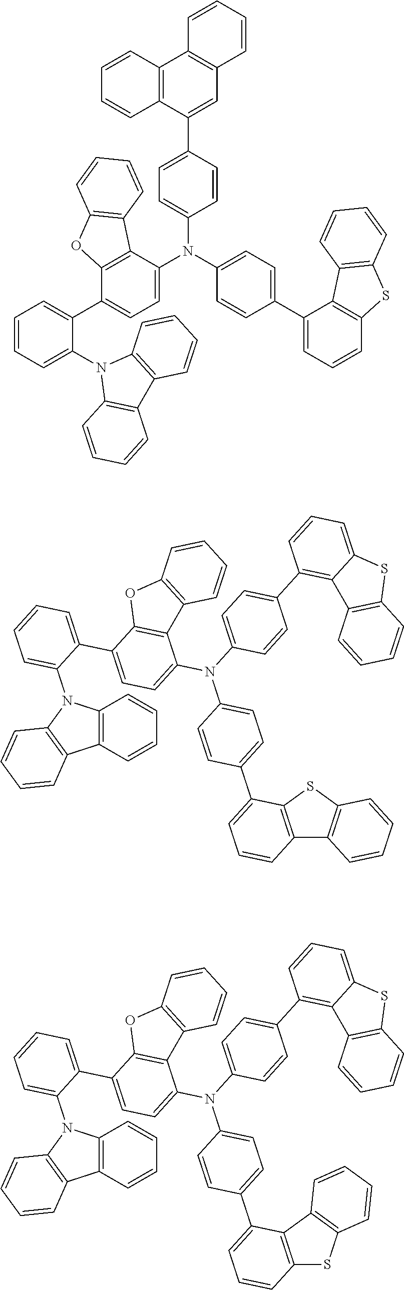 [SMILES: c1ccc(-n2c3ccccc3c3ccccc32)c(-c2ccc(N(c3ccc(-c4cc5ccccc5c5ccccc45)cc3)c3ccc(-c4cccc5sc6ccccc6c45)cc3)c3c2oc2ccccc23)c1 ; c1ccc(-n2c3ccccc3c3ccccc32)c(-c2ccc(N(c3ccc(-c4cccc5c4sc4ccccc45)cc3)c3ccc(-c4cccc5sc6ccccc6c45)cc3)c3c2oc2ccccc23)c1 ; c1ccc(-n2c3ccccc3c3ccccc32)c(-c2ccc(N(c3ccc(-c4cccc5sc6ccccc6c45)cc3)c3ccc(-c4cccc5sc6ccccc6c45)cc3)c3c2oc2ccccc23)c1]

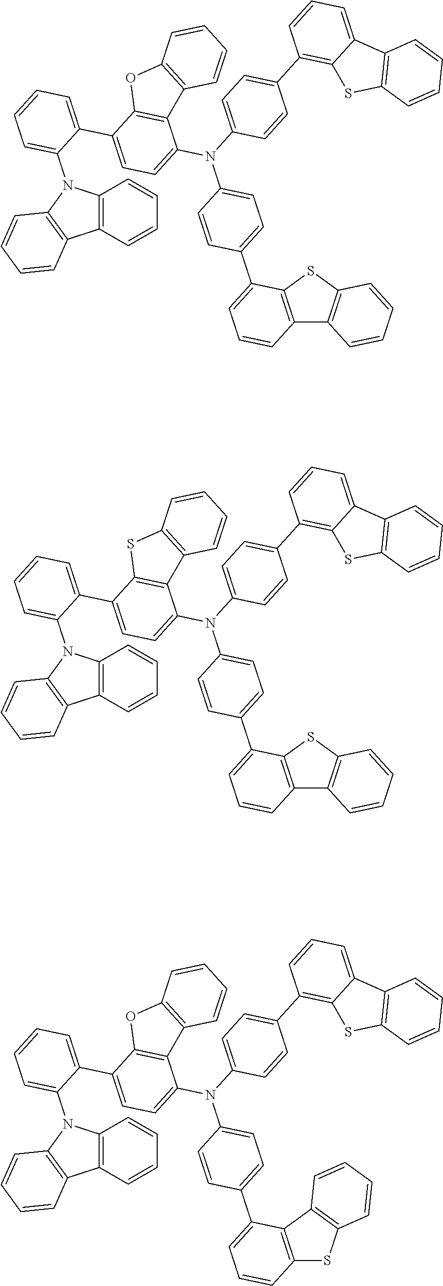 [SMILES: c1ccc(-n2c3ccccc3c3ccccc32)c(-c2ccc(N(c3ccc(-c4cccc5c4sc4ccccc45)cc3)c3ccc(-c4cccc5c4sc4ccccc45)cc3)c3c2oc2ccccc23)c1 ; c1ccc(-n2c3ccccc3c3ccccc32)c(-c2ccc(N(c3ccc(-c4cccc5c4sc4ccccc45)cc3)c3ccc(-c4cccc5c4sc4ccccc45)cc3)c3c2sc2ccccc23)c1 ; c1ccc(-n2c3ccccc3c3ccccc32)c(-c2ccc(N(c3ccc(-c4cccc5c4sc4ccccc45)cc3)c3ccc(-c4cccc5sc6ccccc6c45)cc3)c3c2oc2ccccc23)c1]

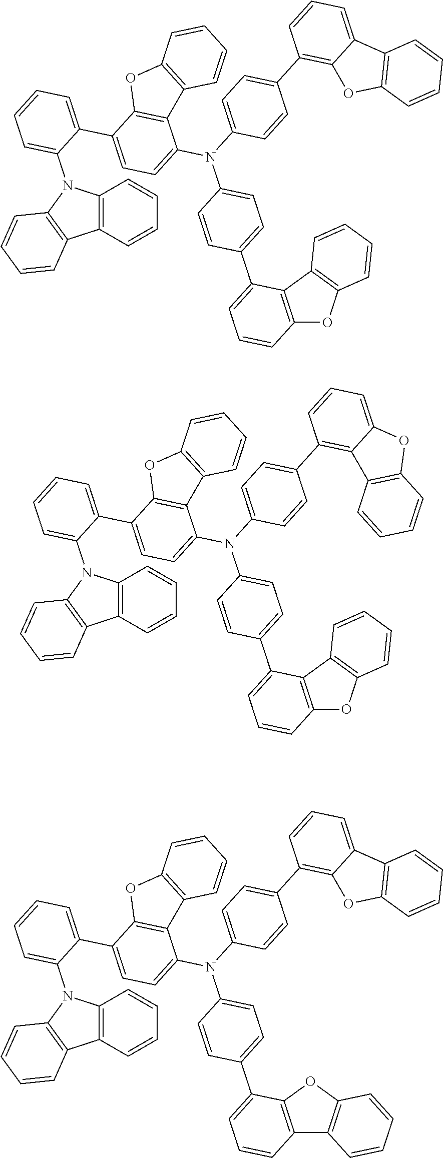 [SMILES: c1ccc(-n2c3ccccc3c3ccccc32)c(-c2ccc(N(c3ccc(-c4cccc5c4oc4ccccc45)cc3)c3ccc(-c4cccc5oc6ccccc6c45)cc3)c3c2oc2ccccc23)c1 ; c1ccc(-n2c3ccccc3c3ccccc32)c(-c2ccc(N(c3ccc(-c4cccc5oc6ccccc6c45)cc3)c3ccc(-c4cccc5oc6ccccc6c45)cc3)c3c2oc2ccccc23)c1 ; c1ccc(-n2c3ccccc3c3ccccc32)c(-c2ccc(N(c3ccc(-c4cccc5c4oc4ccccc45)cc3)c3ccc(-c4cccc5c4oc4ccccc45)cc3)c3c2oc2ccccc23)c1]

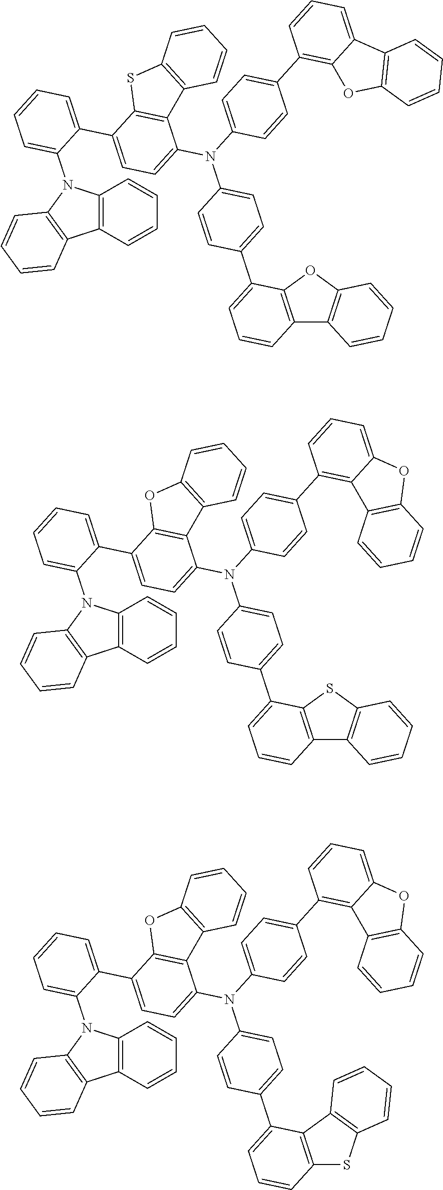 [SMILES: c1ccc(-n2c3ccccc3c3ccccc32)c(-c2ccc(N(c3ccc(-c4cccc5c4oc4ccccc45)cc3)c3ccc(-c4cccc5c4oc4ccccc45)cc3)c3c2sc2ccccc23)c1 ; c1ccc(-n2c3ccccc3c3ccccc32)c(-c2ccc(N(c3ccc(-c4cccc5c4sc4ccccc45)cc3)c3ccc(-c4cccc5oc6ccccc6c45)cc3)c3c2oc2ccccc23)c1 ; c1ccc(-n2c3ccccc3c3ccccc32)c(-c2ccc(N(c3ccc(-c4cccc5oc6ccccc6c45)cc3)c3ccc(-c4cccc5sc6ccccc6c45)cc3)c3c2oc2ccccc23)c1]

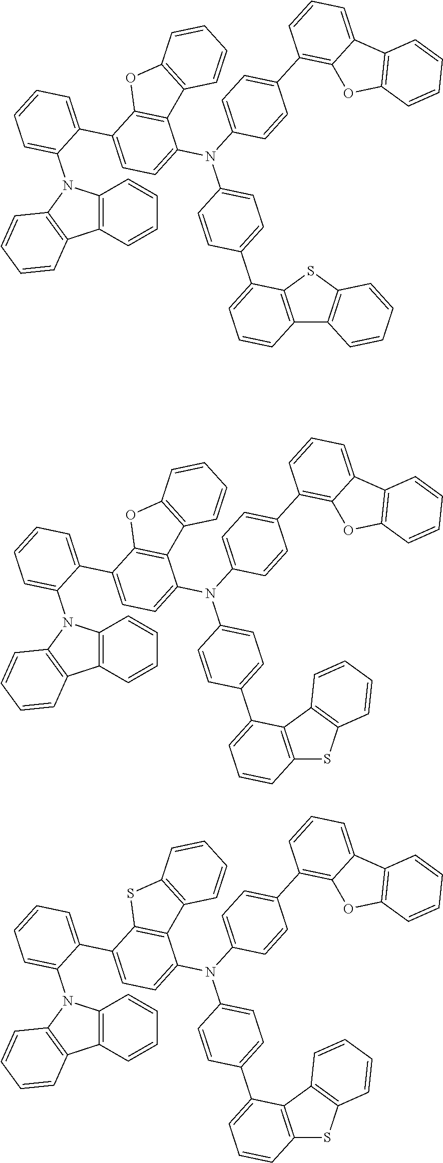 [SMILES: c1ccc(-n2c3ccccc3c3ccccc32)c(-c2ccc(N(c3ccc(-c4cccc5c4oc4ccccc45)cc3)c3ccc(-c4cccc5c4sc4ccccc45)cc3)c3c2oc2ccccc23)c1 ; c1ccc(-n2c3ccccc3c3ccccc32)c(-c2ccc(N(c3ccc(-c4cccc5c4oc4ccccc45)cc3)c3ccc(-c4cccc5sc6ccccc6c45)cc3)c3c2oc2ccccc23)c1 ; c1ccc(-n2c3ccccc3c3ccccc32)c(-c2ccc(N(c3ccc(-c4cccc5c4oc4ccccc45)cc3)c3ccc(-c4cccc5sc6ccccc6c45)cc3)c3c2sc2ccccc23)c1]

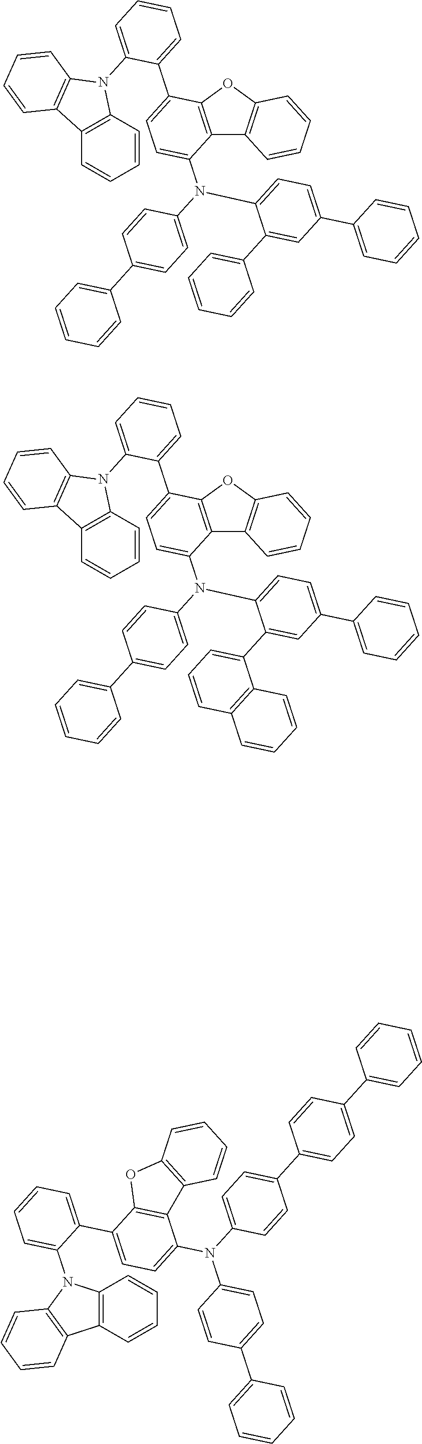 [SMILES: c1ccc(-c2ccc(N(c3ccc(-c4ccccc4)cc3-c3ccccc3)c3ccc(-c4ccccc4-n4c5ccccc5c5ccccc54)c4oc5ccccc5c34)cc2)cc1 ; c1ccc(-c2ccc(N(c3ccc(-c4ccccc4)cc3-c3cccc4ccccc34)c3ccc(-c4ccccc4-n4c5ccccc5c5ccccc54)c4oc5ccccc5c34)cc2)cc1 ; c1ccc(-c2ccc(-c3ccc(N(c4ccc(-c5ccccc5)cc4)c4ccc(-c5ccccc5-n5c6ccccc6c6ccccc65)c5oc6ccccc6c45)cc3)cc2)cc1]

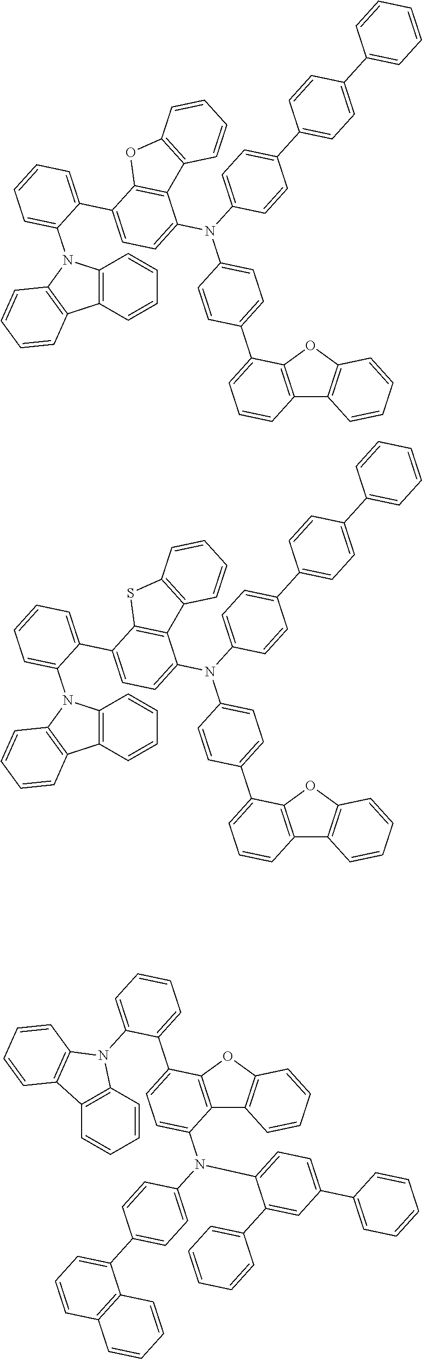

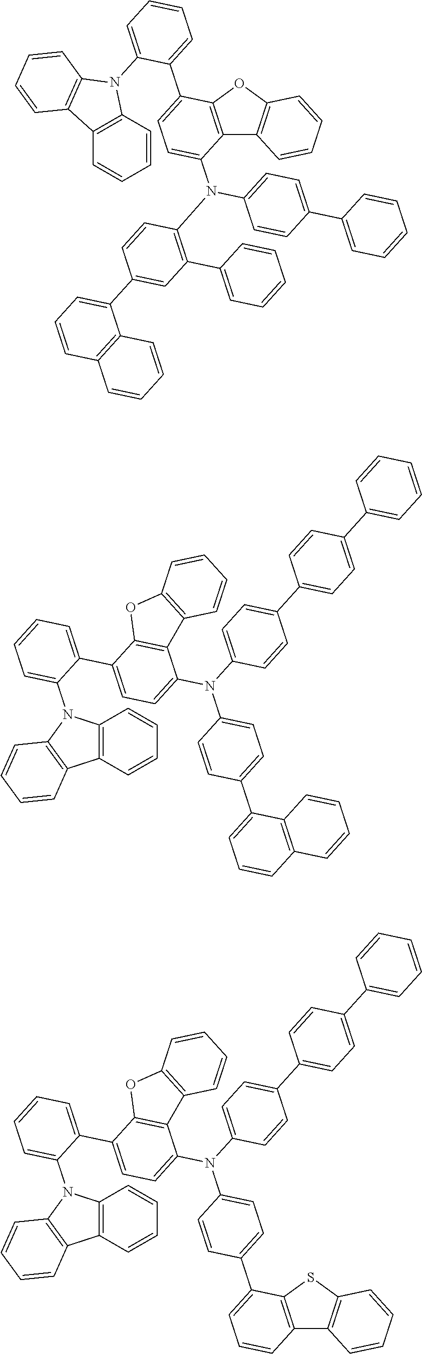 [SMILES: c1ccc(-c2ccc(N(c3ccc(-c4cccc5ccccc45)cc3-c3ccccc3)c3ccc(-c4ccccc4-n4c5ccccc5c5ccccc54)c4oc5ccccc5c34)cc2)cc1 ; c1ccc(-c2ccc(-c3ccc(N(c4ccc(-c5cccc6ccccc56)cc4)c4ccc(-c5ccccc5-n5c6ccccc6c6ccccc65)c5oc6ccccc6c45)cc3)cc2)cc1 ; c1ccc(-c2ccc(-c3ccc(N(c4ccc(-c5cccc6c5sc5ccccc56)cc4)c4ccc(-c5ccccc5-n5c6ccccc6c6ccccc65)c5oc6ccccc6c45)cc3)cc2)cc1]

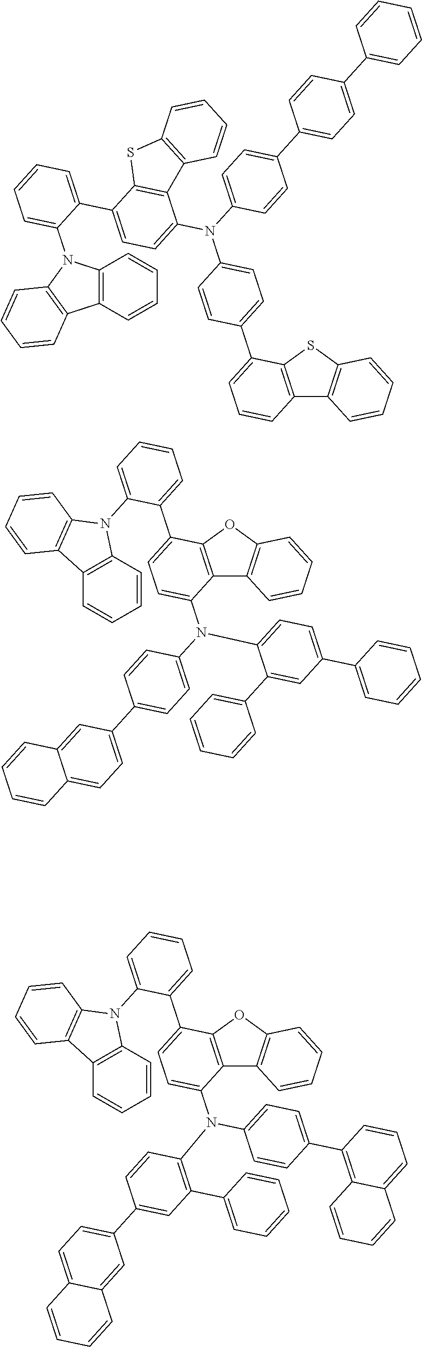 [SMILES: c1ccc(-c2ccc(-c3ccc(N(c4ccc(-c5cccc6c5sc5ccccc56)cc4)c4ccc(-c5ccccc5-n5c6ccccc6c6ccccc65)c5sc6ccccc6c45)cc3)cc2)cc1 ; c1ccc(-c2ccc(N(c3ccc(-c4ccc5ccccc5c4)cc3)c3ccc(-c4ccccc4-n4c5ccccc5c5ccccc54)c4oc5ccccc5c34)c(-c3ccccc3)c2)cc1 ; c1ccc(-c2cc(-c3ccc4ccccc4c3)ccc2N(c2ccc(-c3cccc4ccccc34)cc2)c2ccc(-c3ccccc3-n3c4ccccc4c4ccccc43)c3oc4ccccc4c23)cc1]

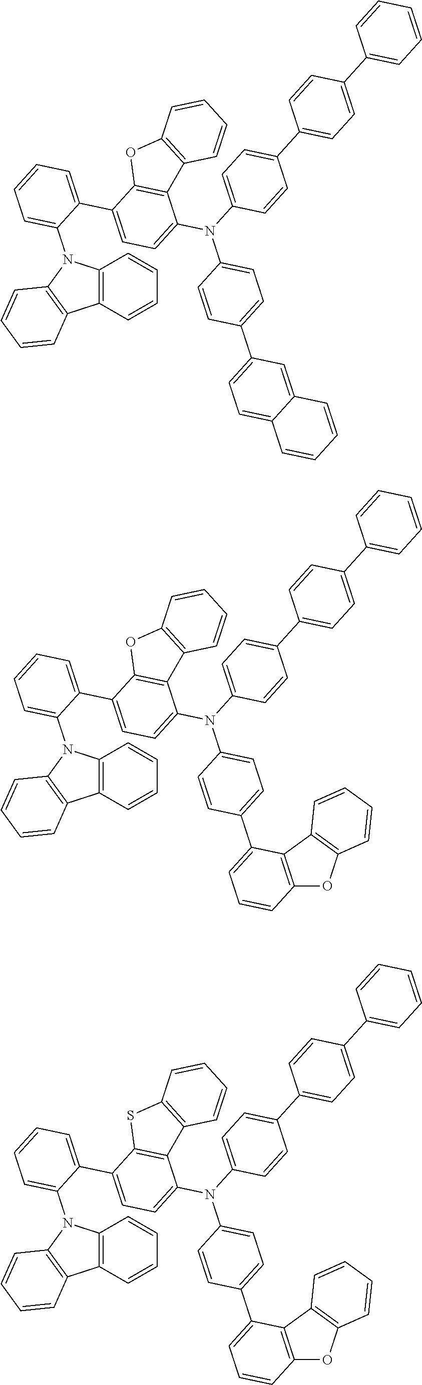 [SMILES: c1ccc(-c2ccc(-c3ccc(N(c4ccc(-c5ccc6ccccc6c5)cc4)c4ccc(-c5ccccc5-n5c6ccccc6c6ccccc65)c5oc6ccccc6c45)cc3)cc2)cc1 ; c1ccc(-c2ccc(-c3ccc(N(c4ccc(-c5cccc6oc7ccccc7c56)cc4)c4ccc(-c5ccccc5-n5c6ccccc6c6ccccc65)c5oc6ccccc6c45)cc3)cc2)cc1 ; c1ccc(-c2ccc(-c3ccc(N(c4ccc(-c5cccc6oc7ccccc7c56)cc4)c4ccc(-c5ccccc5-n5c6ccccc6c6ccccc65)c5sc6ccccc6c45)cc3)cc2)cc1]

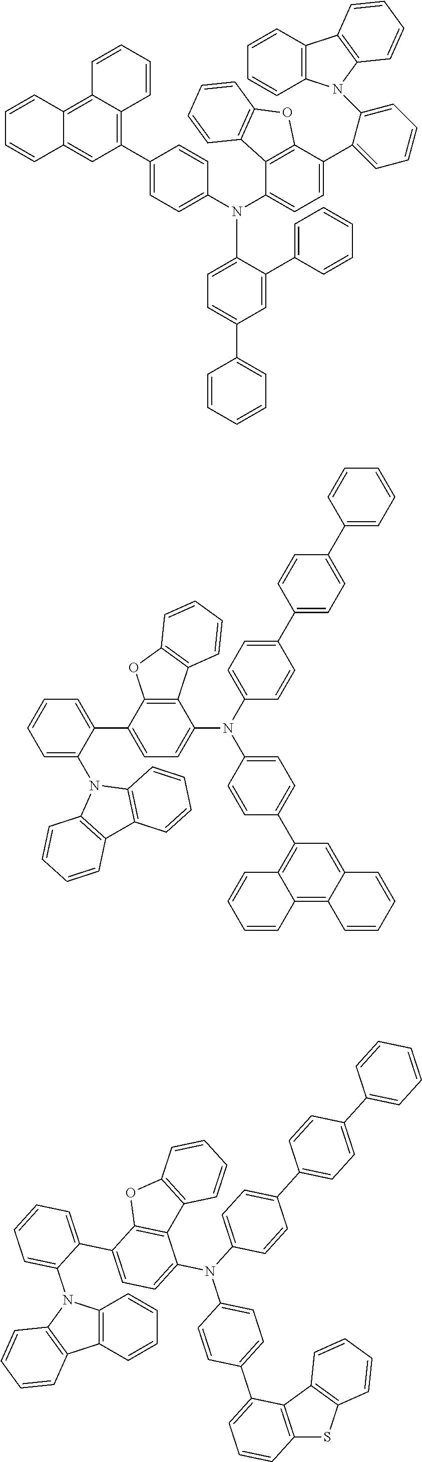 [SMILES: c1ccc(-c2ccc(N(c3ccc(-c4cc5ccccc5c5ccccc45)cc3)c3ccc(-c4ccccc4-n4c5ccccc5c5ccccc54)c4oc5ccccc5c34)c(-c3ccccc3)c2)cc1 ; c1ccc(-c2ccc(-c3ccc(N(c4ccc(-c5cc6ccccc6c6ccccc56)cc4)c4ccc(-c5ccccc5-n5c6ccccc6c6ccccc65)c5oc6ccccc6c45)cc3)cc2)cc1 ; c1ccc(-c2ccc(-c3ccc(N(c4ccc(-c5cccc6sc7ccccc7c56)cc4)c4ccc(-c5ccccc5-n5c6ccccc6c6ccccc65)c5oc6ccccc6c45)cc3)cc2)cc1]

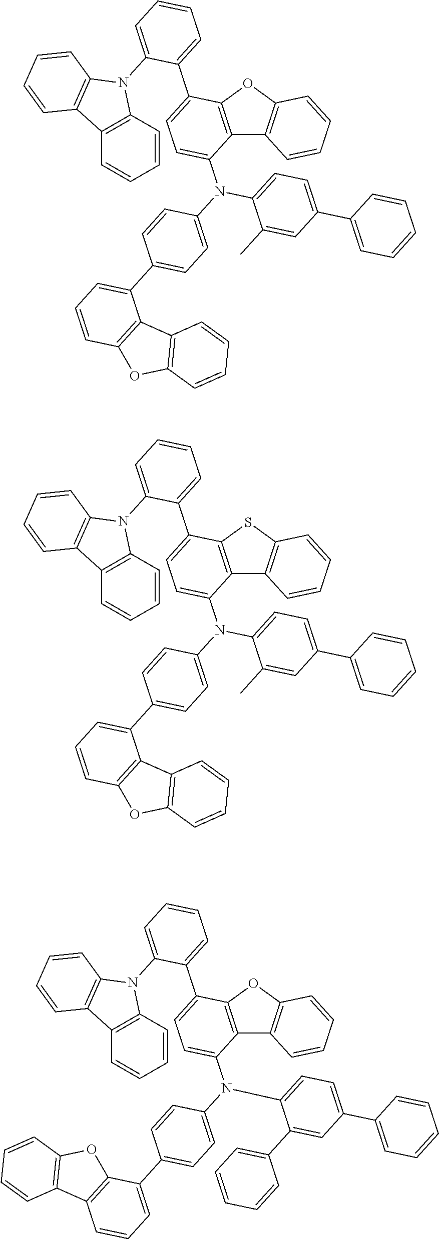 [SMILES: Cc1cc(-c2ccccc2)ccc1N(c1ccc(-c2cccc3oc4ccccc4c23)cc1)c1ccc(-c2ccccc2-n2c3ccccc3c3ccccc32)c2oc3ccccc3c12 ; Cc1cc(-c2ccccc2)ccc1N(c1ccc(-c2cccc3oc4ccccc4c23)cc1)c1ccc(-c2ccccc2-n2c3ccccc3c3ccccc32)c2sc3ccccc3c12 ; c1ccc(-c2ccc(N(c3ccc(-c4cccc5c4oc4ccccc45)cc3)c3ccc(-c4ccccc4-n4c5ccccc5c5ccccc54)c4oc5ccccc5c34)c(-c3ccccc3)c2)cc1]

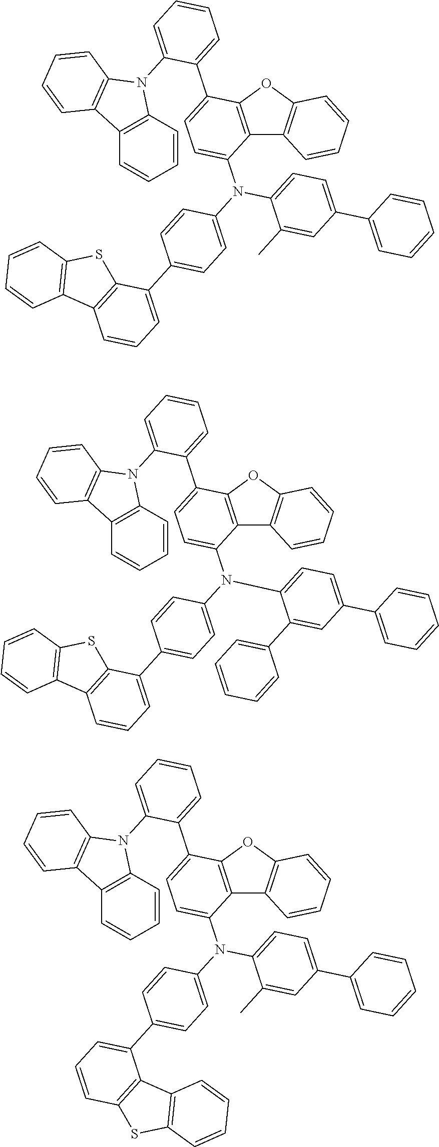

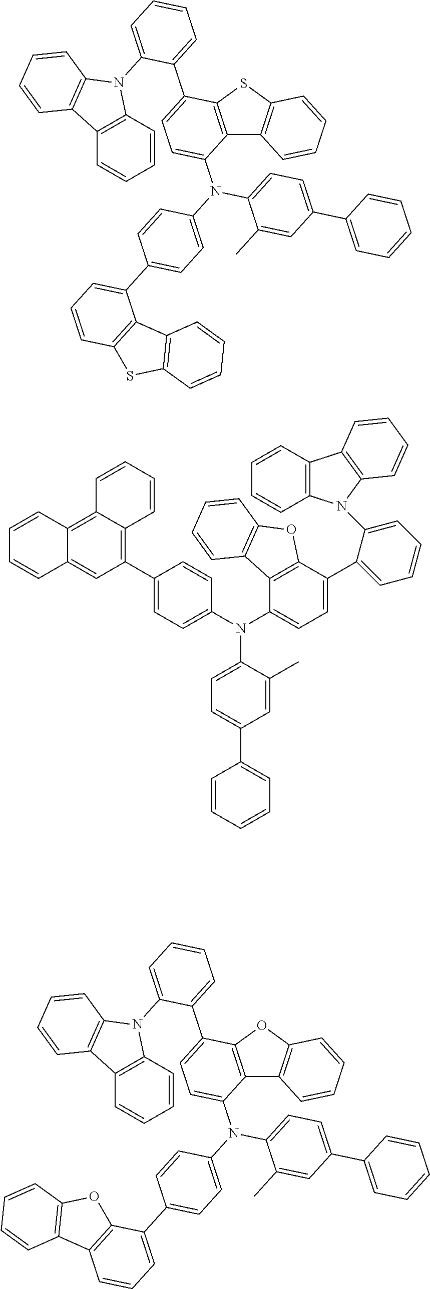 [SMILES: Cc1cc(-c2ccccc2)ccc1N(c1ccc(-c2cccc3sc4ccccc4c23)cc1)c1ccc(-c2ccccc2-n2c3ccccc3c3ccccc32)c2sc3ccccc3c12 ; Cc1cc(-c2ccccc2)ccc1N(c1ccc(-c2cc3ccccc3c3ccccc23)cc1)c1ccc(-c2ccccc2-n2c3ccccc3c3ccccc32)c2oc3ccccc3c12 ; Cc1cc(-c2ccccc2)ccc1N(c1ccc(-c2cccc3c2oc2ccccc23)cc1)c1ccc(-c2ccccc2-n2c3ccccc3c3ccccc32)c2oc3ccccc3c12]

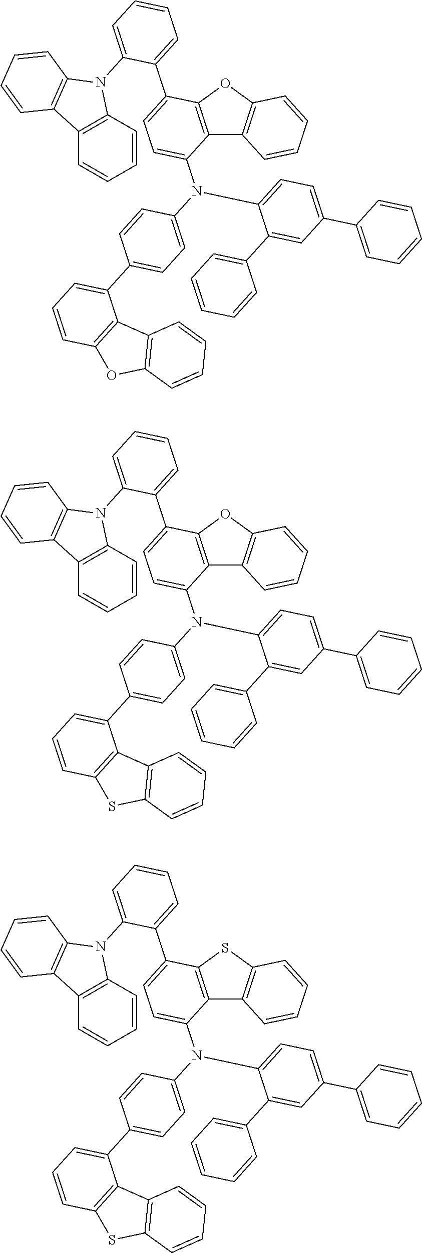 [SMILES: c1ccc(-c2ccc(N(c3ccc(-c4cccc5oc6ccccc6c45)cc3)c3ccc(-c4ccccc4-n4c5ccccc5c5ccccc54)c4oc5ccccc5c34)c(-c3ccccc3)c2)cc1 ; c1ccc(-c2ccc(N(c3ccc(-c4cccc5sc6ccccc6c45)cc3)c3ccc(-c4ccccc4-n4c5ccccc5c5ccccc54)c4oc5ccccc5c34)c(-c3ccccc3)c2)cc1 ; c1ccc(-c2ccc(N(c3ccc(-c4cccc5sc6ccccc6c45)cc3)c3ccc(-c4ccccc4-n4c5ccccc5c5ccccc54)c4sc5ccccc5c34)c(-c3ccccc3)c2)cc1]

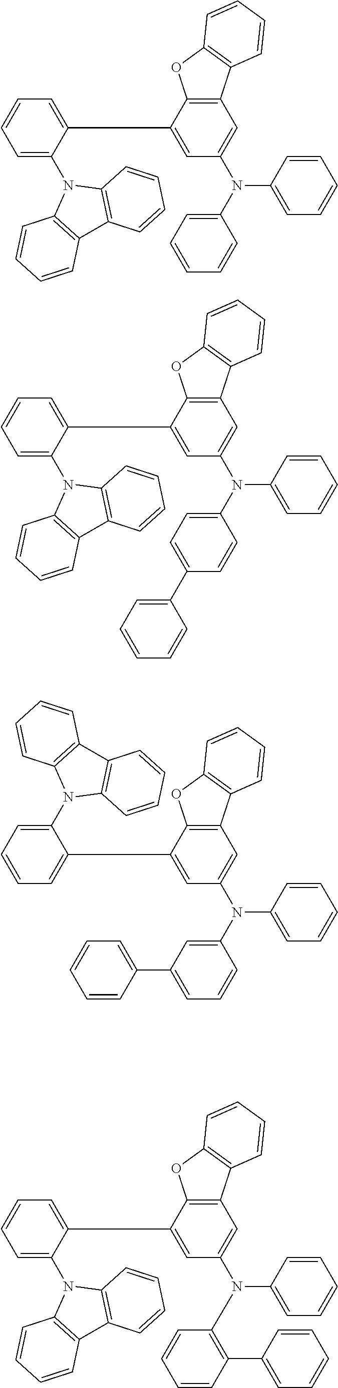

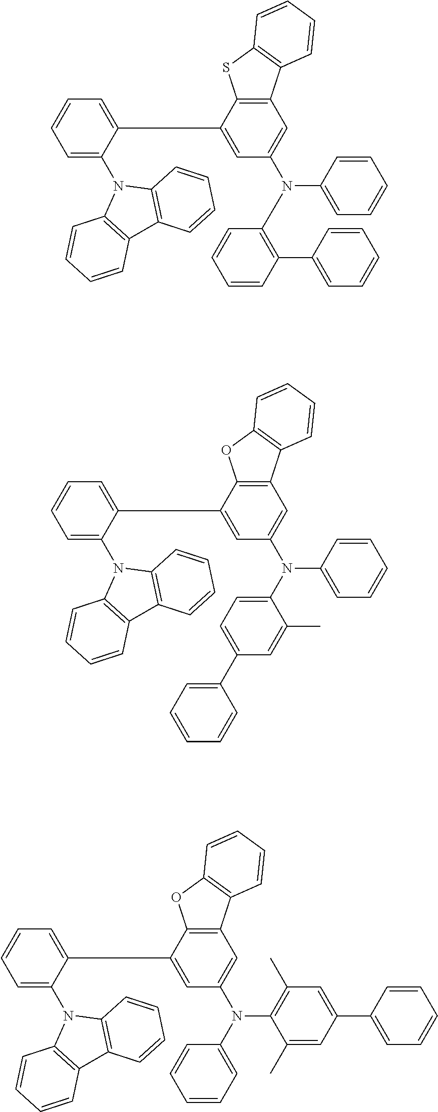 [SMILES: c1ccc(-c2ccccc2N(c2ccccc2)c2cc(-c3ccccc3-n3c4ccccc4c4ccccc43)c3sc4ccccc4c3c2)cc1 ; Cc1cc(-c2ccccc2)ccc1N(c1ccccc1)c1cc(-c2ccccc2-n2c3ccccc3c3ccccc32)c2oc3ccccc3c2c1 ; Cc1cc(-c2ccccc2)cc(C)c1N(c1ccccc1)c1cc(-c2ccccc2-n2c3ccccc3c3ccccc32)c2oc3ccccc3c2c1]

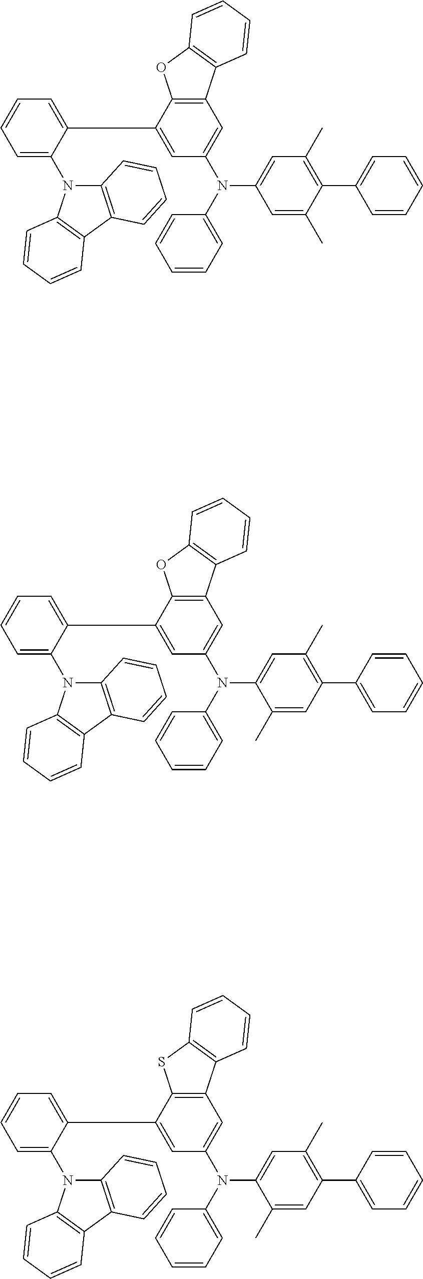 [SMILES: Cc1cc(N(c2ccccc2)c2cc(-c3ccccc3-n3c4ccccc4c4ccccc43)c3oc4ccccc4c3c2)cc(C)c1-c1ccccc1 ; Cc1cc(N(c2ccccc2)c2cc(-c3ccccc3-n3c4ccccc4c4ccccc43)c3oc4ccccc4c3c2)c(C)cc1-c1ccccc1 ; Cc1cc(N(c2ccccc2)c2cc(-c3ccccc3-n3c4ccccc4c4ccccc43)c3sc4ccccc4c3c2)c(C)cc1-c1ccccc1]

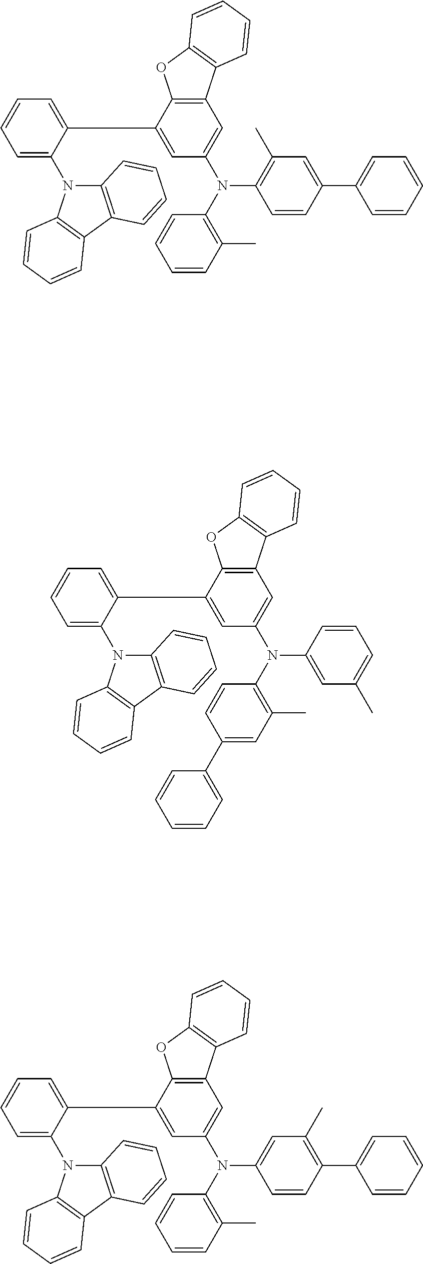 [SMILES: Cc1ccccc1N(c1cc(-c2ccccc2-n2c3ccccc3c3ccccc32)c2oc3ccccc3c2c1)c1ccc(-c2ccccc2)cc1C ; Cc1cccc(N(c2cc(-c3ccccc3-n3c4ccccc4c4ccccc43)c3oc4ccccc4c3c2)c2ccc(-c3ccccc3)cc2C)c1 ; Cc1cc(N(c2cc(-c3ccccc3-n3c4ccccc4c4ccccc43)c3oc4ccccc4c3c2)c2ccccc2C)ccc1-c1ccccc1]

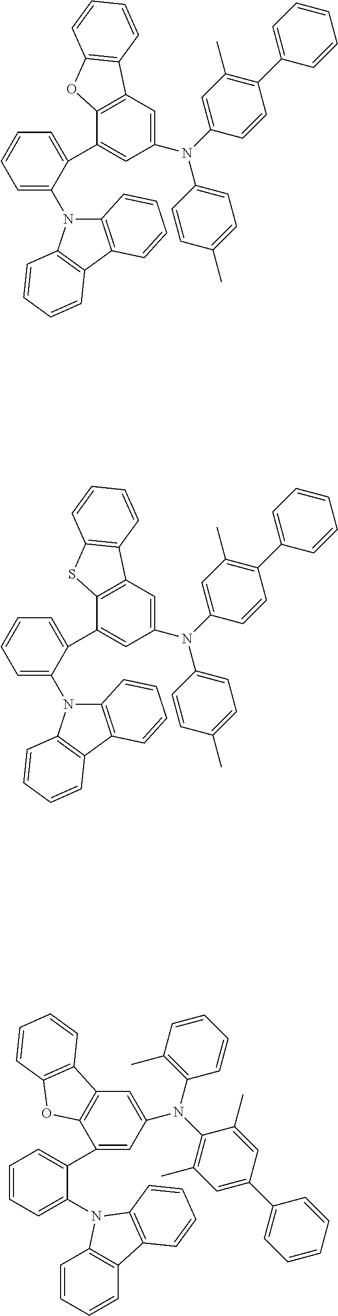 [SMILES: Cc1ccc(N(c2ccc(-c3ccccc3)c(C)c2)c2cc(-c3ccccc3-n3c4ccccc4c4ccccc43)c3oc4ccccc4c3c2)cc1 ; Cc1ccc(N(c2ccc(-c3ccccc3)c(C)c2)c2cc(-c3ccccc3-n3c4ccccc4c4ccccc43)c3sc4ccccc4c3c2)cc1 ; Cc1ccccc1N(c1cc(-c2ccccc2-n2c3ccccc3c3ccccc32)c2oc3ccccc3c2c1)c1c(C)cc(-c2ccccc2)cc1C]

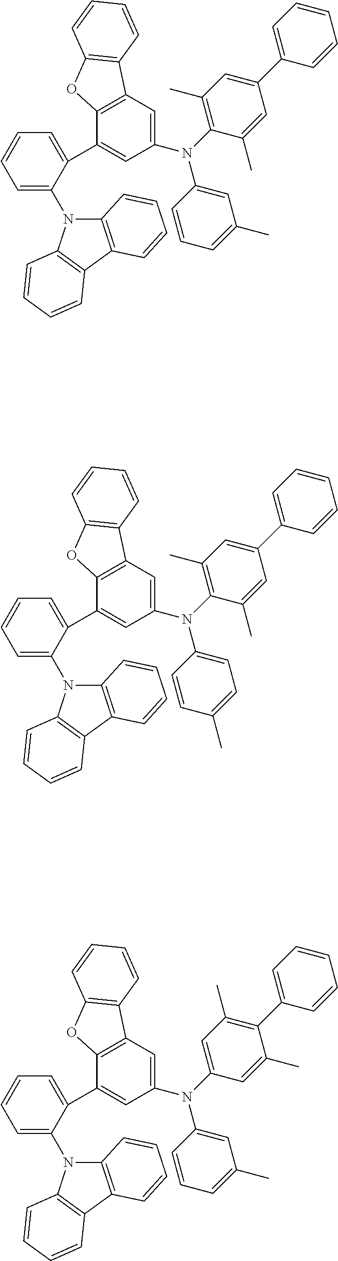 [SMILES: Cc1cccc(N(c2cc(-c3ccccc3-n3c4ccccc4c4ccccc43)c3oc4ccccc4c3c2)c2c(C)cc(-c3ccccc3)cc2C)c1 ; Cc1ccc(N(c2cc(-c3ccccc3-n3c4ccccc4c4ccccc43)c3oc4ccccc4c3c2)c2c(C)cc(-c3ccccc3)cc2C)cc1 ; Cc1cccc(N(c2cc(C)c(-c3ccccc3)c(C)c2)c2cc(-c3ccccc3-n3c4ccccc4c4ccccc43)c3oc4ccccc4c3c2)c1]

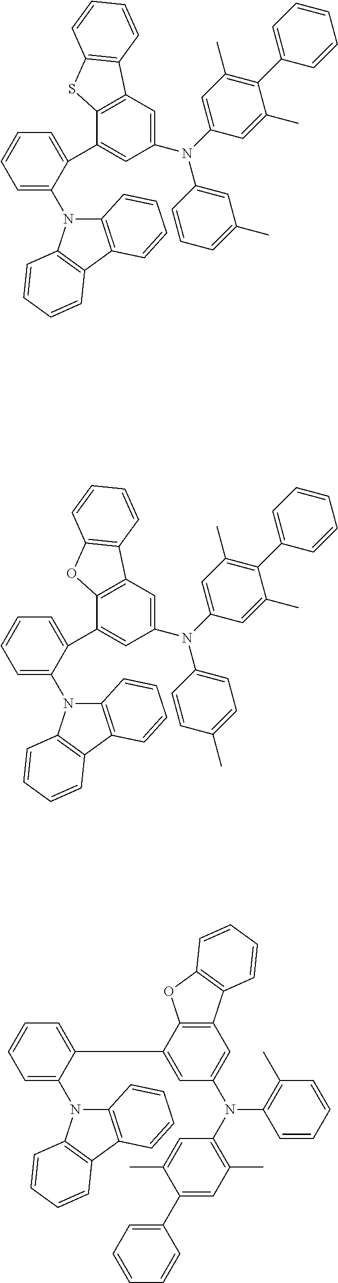 [SMILES: Cc1cccc(N(c2cc(C)c(-c3ccccc3)c(C)c2)c2cc(-c3ccccc3-n3c4ccccc4c4ccccc43)c3sc4ccccc4c3c2)c1 ; Cc1ccc(N(c2cc(C)c(-c3ccccc3)c(C)c2)c2cc(-c3ccccc3-n3c4ccccc4c4ccccc43)c3oc4ccccc4c3c2)cc1 ; Cc1cc(N(c2cc(-c3ccccc3-n3c4ccccc4c4ccccc43)c3oc4ccccc4c3c2)c2ccccc2C)c(C)cc1-c1ccccc1]

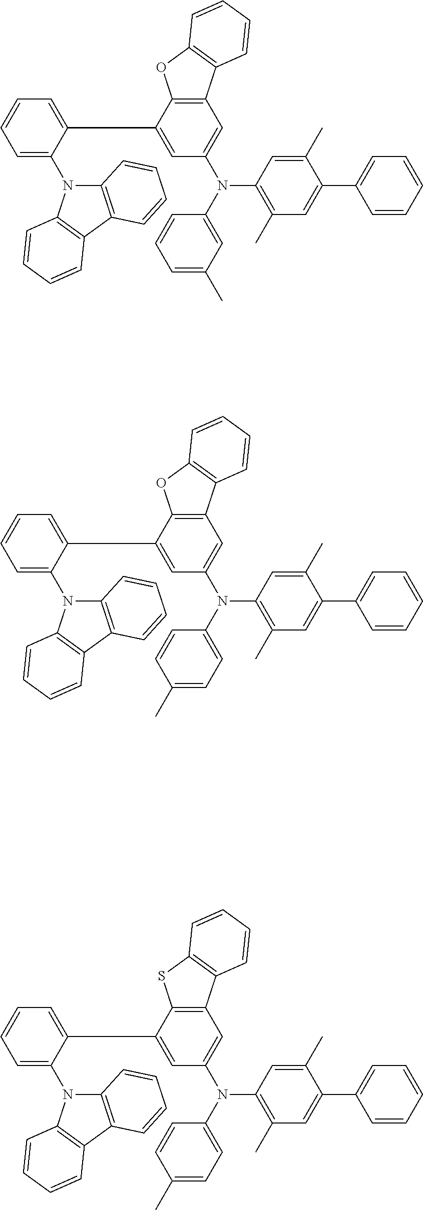 [SMILES: Cc1cccc(N(c2cc(-c3ccccc3-n3c4ccccc4c4ccccc43)c3oc4ccccc4c3c2)c2cc(C)c(-c3ccccc3)cc2C)c1 ; Cc1ccc(N(c2cc(-c3ccccc3-n3c4ccccc4c4ccccc43)c3oc4ccccc4c3c2)c2cc(C)c(-c3ccccc3)cc2C)cc1 ; Cc1ccc(N(c2cc(-c3ccccc3-n3c4ccccc4c4ccccc43)c3sc4ccccc4c3c2)c2cc(C)c(-c3ccccc3)cc2C)cc1]

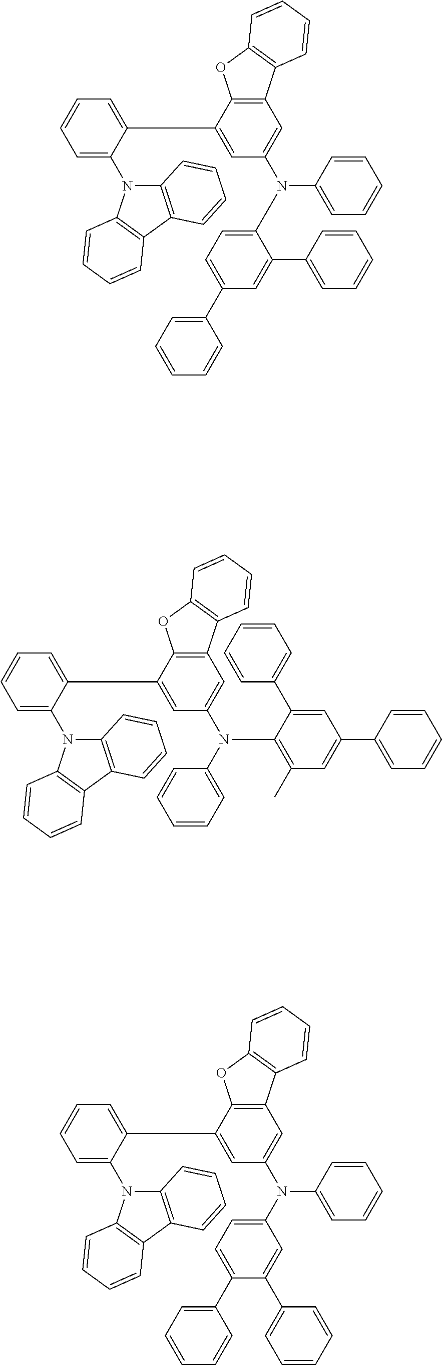 [SMILES: c1ccc(-c2ccc(N(c3ccccc3)c3cc(-c4ccccc4-n4c5ccccc5c5ccccc54)c4oc5ccccc5c4c3)c(-c3ccccc3)c2)cc1 ; Cc1cc(-c2ccccc2)cc(-c2ccccc2)c1N(c1ccccc1)c1cc(-c2ccccc2-n2c3ccccc3c3ccccc32)c2oc3ccccc3c2c1 ; c1ccc(-c2ccc(N(c3ccccc3)c3cc(-c4ccccc4-n4c5ccccc5c5ccccc54)c4oc5ccccc5c4c3)cc2-c2ccccc2)cc1]

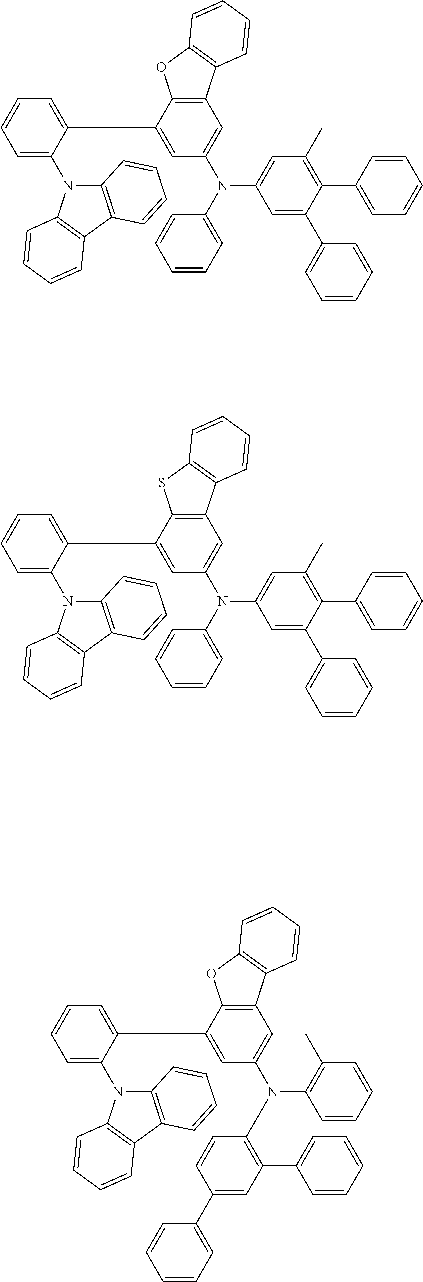 [SMILES: Cc1cc(N(c2ccccc2)c2cc(-c3ccccc3-n3c4ccccc4c4ccccc43)c3oc4ccccc4c3c2)cc(-c2ccccc2)c1-c1ccccc1 ; Cc1cc(N(c2ccccc2)c2cc(-c3ccccc3-n3c4ccccc4c4ccccc43)c3sc4ccccc4c3c2)cc(-c2ccccc2)c1-c1ccccc1 ; Cc1ccccc1N(c1cc(-c2ccccc2-n2c3ccccc3c3ccccc32)c2oc3ccccc3c2c1)c1ccc(-c2ccccc2)cc1-c1ccccc1]

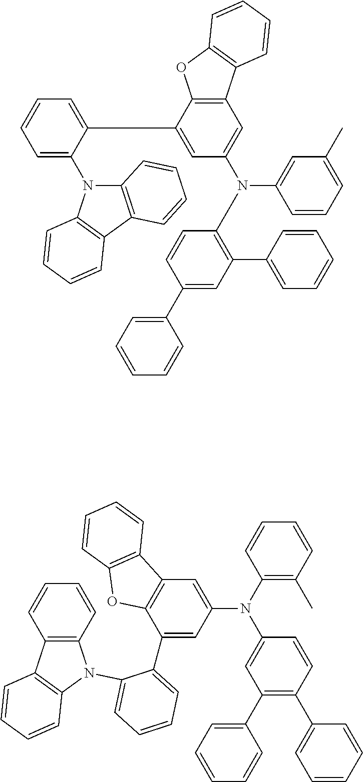 [SMILES: Cc1cccc(N(c2cc(-c3ccccc3-n3c4ccccc4c4ccccc43)c3oc4ccccc4c3c2)c2ccc(-c3ccccc3)cc2-c2ccccc2)c1 ; Cc1ccccc1N(c1ccc(-c2ccccc2)c(-c2ccccc2)c1)c1cc(-c2ccccc2-n2c3ccccc3c3ccccc32)c2oc3ccccc3c2c1]

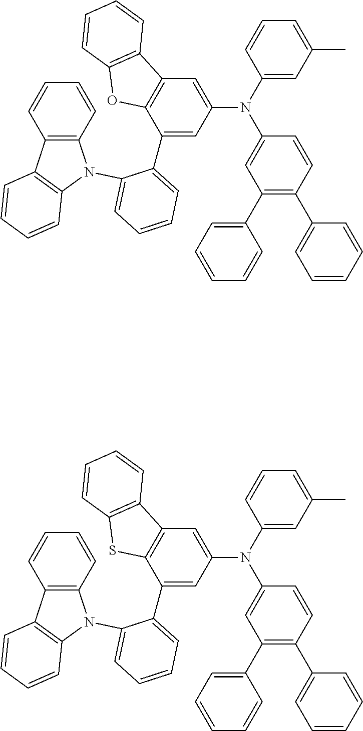 [SMILES: Cc1cccc(N(c2ccc(-c3ccccc3)c(-c3ccccc3)c2)c2cc(-c3ccccc3-n3c4ccccc4c4ccccc43)c3oc4ccccc4c3c2)c1 ; Cc1cccc(N(c2ccc(-c3ccccc3)c(-c3ccccc3)c2)c2cc(-c3ccccc3-n3c4ccccc4c4ccccc43)c3sc4ccccc4c3c2)c1]

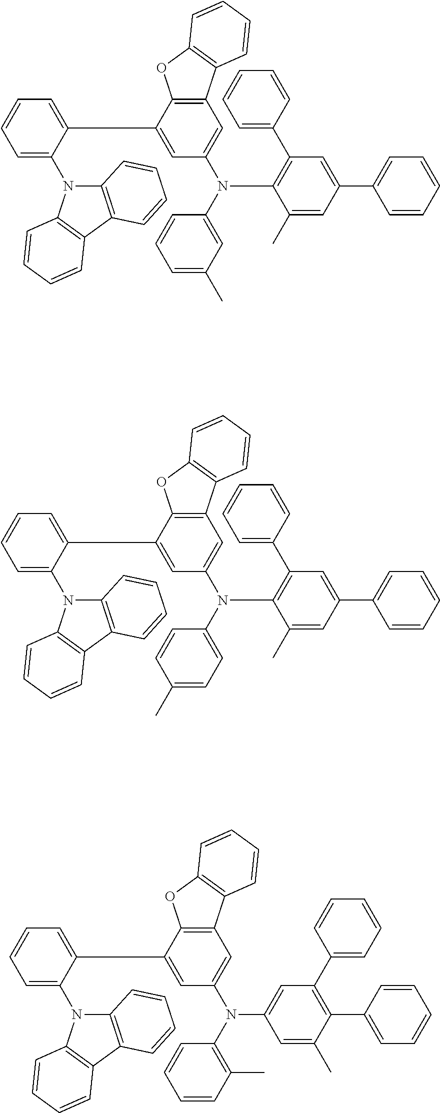 [SMILES: Cc1cccc(N(c2cc(-c3ccccc3-n3c4ccccc4c4ccccc43)c3oc4ccccc4c3c2)c2c(C)cc(-c3ccccc3)cc2-c2ccccc2)c1 ; Cc1ccc(N(c2cc(-c3ccccc3-n3c4ccccc4c4ccccc43)c3oc4ccccc4c3c2)c2c(C)cc(-c3ccccc3)cc2-c2ccccc2)cc1 ; Cc1ccccc1N(c1cc(C)c(-c2ccccc2)c(-c2ccccc2)c1)c1cc(-c2ccccc2-n2c3ccccc3c3ccccc32)c2oc3ccccc3c2c1]

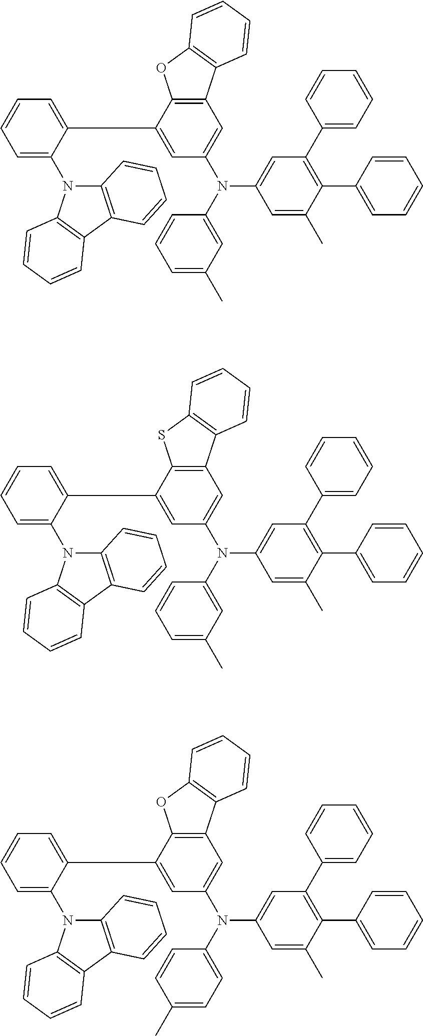 [SMILES: Cc1cccc(N(c2cc(C)c(-c3ccccc3)c(-c3ccccc3)c2)c2cc(-c3ccccc3-n3c4ccccc4c4ccccc43)c3oc4ccccc4c3c2)c1 ; Cc1cccc(N(c2cc(C)c(-c3ccccc3)c(-c3ccccc3)c2)c2cc(-c3ccccc3-n3c4ccccc4c4ccccc43)c3sc4ccccc4c3c2)c1 ; Cc1ccc(N(c2cc(C)c(-c3ccccc3)c(-c3ccccc3)c2)c2cc(-c3ccccc3-n3c4ccccc4c4ccccc43)c3oc4ccccc4c3c2)cc1]

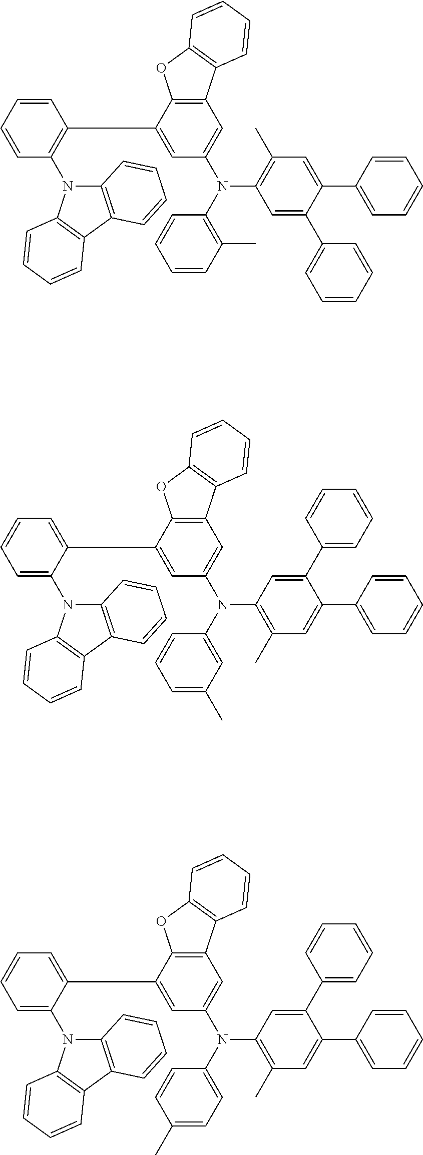 [SMILES: Cc1ccccc1N(c1cc(-c2ccccc2-n2c3ccccc3c3ccccc32)c2oc3ccccc3c2c1)c1cc(-c2ccccc2)c(-c2ccccc2)cc1C ; Cc1cccc(N(c2cc(-c3ccccc3-n3c4ccccc4c4ccccc43)c3oc4ccccc4c3c2)c2cc(-c3ccccc3)c(-c3ccccc3)cc2C)c1 ; Cc1ccc(N(c2cc(-c3ccccc3-n3c4ccccc4c4ccccc43)c3oc4ccccc4c3c2)c2cc(-c3ccccc3)c(-c3ccccc3)cc2C)cc1]

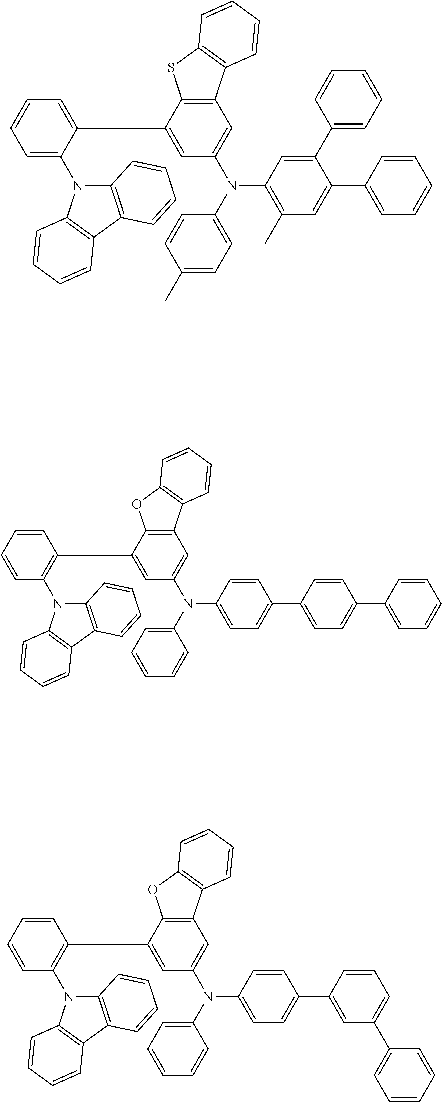 [SMILES: Cc1ccc(N(c2cc(-c3ccccc3-n3c4ccccc4c4ccccc43)c3sc4ccccc4c3c2)c2cc(-c3ccccc3)c(-c3ccccc3)cc2C)cc1 ; c1ccc(-c2ccc(-c3ccc(N(c4ccccc4)c4cc(-c5ccccc5-n5c6ccccc6c6ccccc65)c5oc6ccccc6c5c4)cc3)cc2)cc1 ; c1ccc(-c2cccc(-c3ccc(N(c4ccccc4)c4cc(-c5ccccc5-n5c6ccccc6c6ccccc65)c5oc6ccccc6c5c4)cc3)c2)cc1]

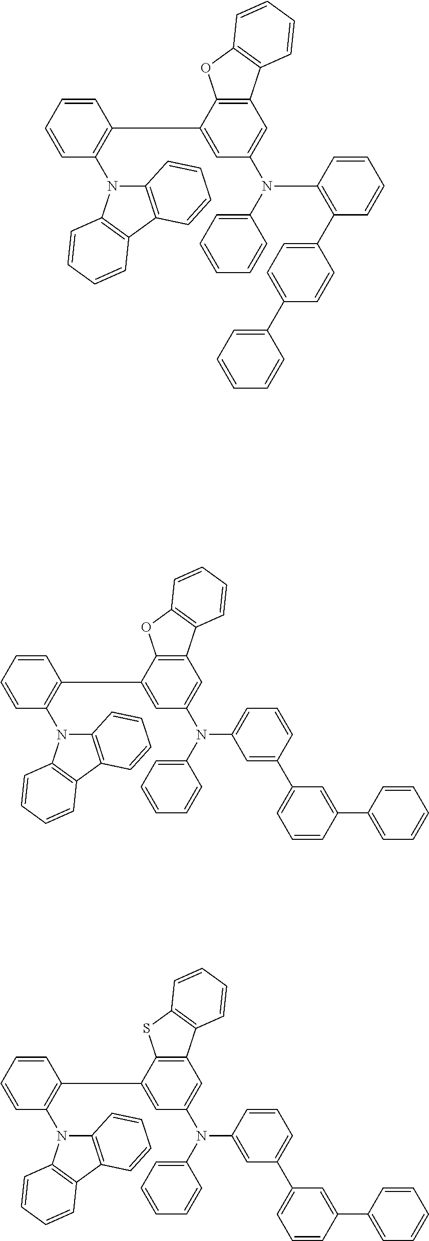 [SMILES: c1ccc(-c2ccc(-c3ccccc3N(c3ccccc3)c3cc(-c4ccccc4-n4c5ccccc5c5ccccc54)c4oc5ccccc5c4c3)cc2)cc1 ; c1ccc(-c2cccc(-c3cccc(N(c4ccccc4)c4cc(-c5ccccc5-n5c6ccccc6c6ccccc65)c5oc6ccccc6c5c4)c3)c2)cc1 ; c1ccc(-c2cccc(-c3cccc(N(c4ccccc4)c4cc(-c5ccccc5-n5c6ccccc6c6ccccc65)c5sc6ccccc6c5c4)c3)c2)cc1]

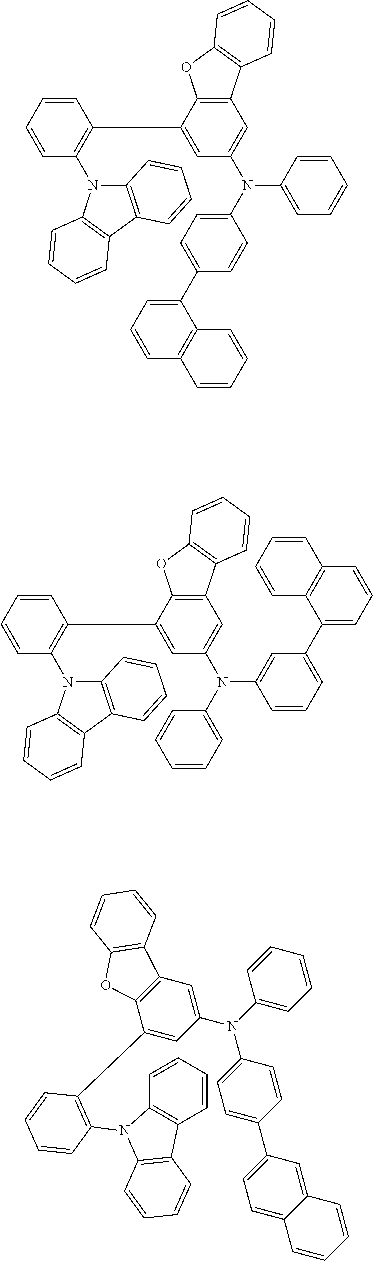 [SMILES: c1ccc(N(c2ccc(-c3cccc4ccccc34)cc2)c2cc(-c3ccccc3-n3c4ccccc4c4ccccc43)c3oc4ccccc4c3c2)cc1 ; c1ccc(N(c2cccc(-c3cccc4ccccc34)c2)c2cc(-c3ccccc3-n3c4ccccc4c4ccccc43)c3oc4ccccc4c3c2)cc1 ; c1ccc(N(c2ccc(-c3ccc4ccccc4c3)cc2)c2cc(-c3ccccc3-n3c4ccccc4c4ccccc43)c3oc4ccccc4c3c2)cc1]

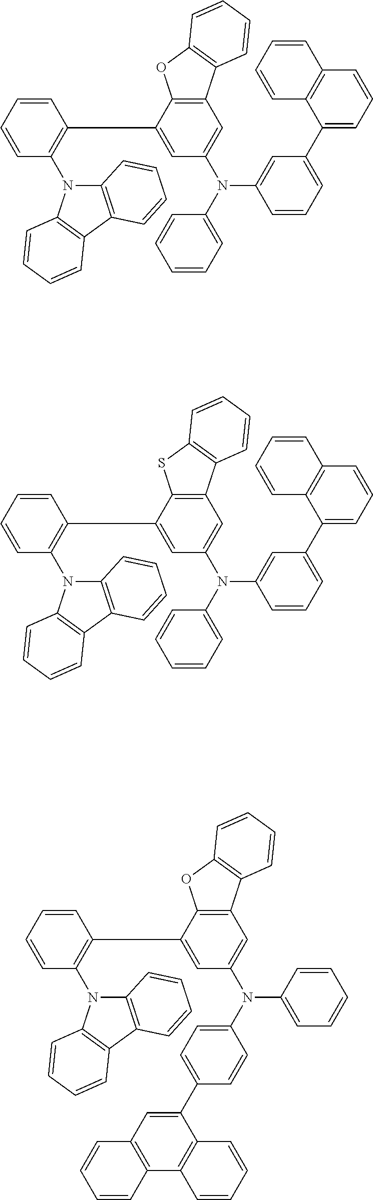 [SMILES: c1ccc(N(c2cccc(-c3cccc4ccccc34)c2)c2cc(-c3ccccc3-n3c4ccccc4c4ccccc43)c3oc4ccccc4c3c2)cc1 ; c1ccc(N(c2cccc(-c3cccc4ccccc34)c2)c2cc(-c3ccccc3-n3c4ccccc4c4ccccc43)c3sc4ccccc4c3c2)cc1 ; c1ccc(N(c2ccc(-c3cc4ccccc4c4ccccc34)cc2)c2cc(-c3ccccc3-n3c4ccccc4c4ccccc43)c3oc4ccccc4c3c2)cc1]

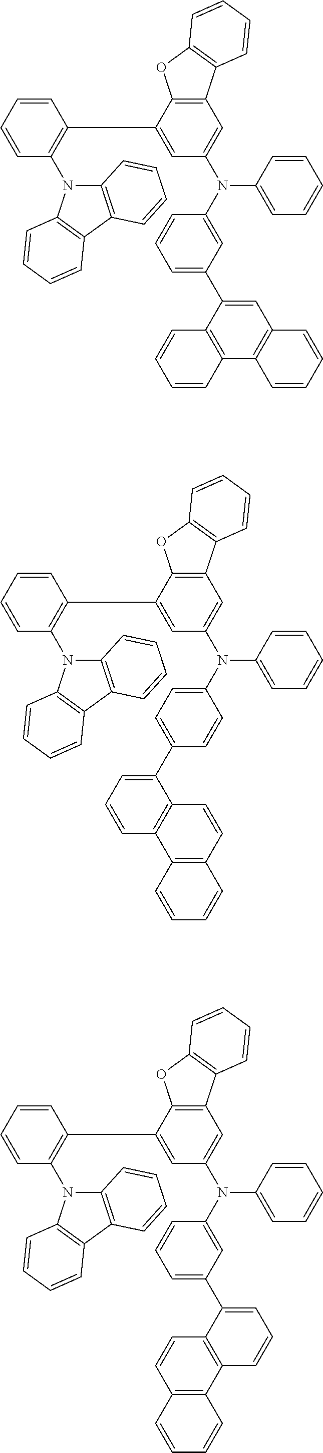 [SMILES: c1ccc(N(c2cccc(-c3cc4ccccc4c4ccccc34)c2)c2cc(-c3ccccc3-n3c4ccccc4c4ccccc43)c3oc4ccccc4c3c2)cc1 ; c1ccc(N(c2ccc(-c3cccc4c3ccc3ccccc34)cc2)c2cc(-c3ccccc3-n3c4ccccc4c4ccccc43)c3oc4ccccc4c3c2)cc1 ; c1ccc(N(c2cccc(-c3cccc4c3ccc3ccccc34)c2)c2cc(-c3ccccc3-n3c4ccccc4c4ccccc43)c3oc4ccccc4c3c2)cc1]

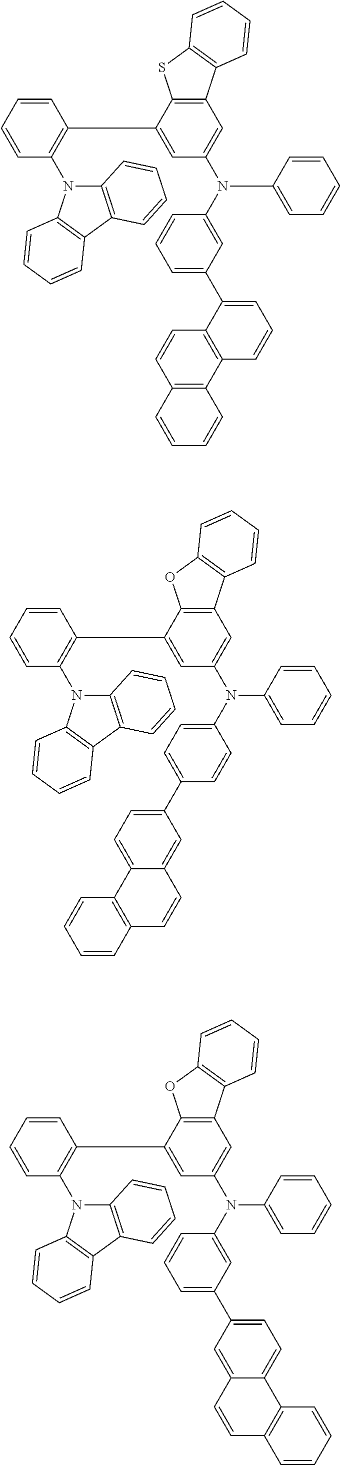 [SMILES: c1ccc(N(c2cccc(-c3cccc4c3ccc3ccccc34)c2)c2cc(-c3ccccc3-n3c4ccccc4c4ccccc43)c3sc4ccccc4c3c2)cc1 ; c1ccc(N(c2ccc(-c3ccc4c(ccc5ccccc54)c3)cc2)c2cc(-c3ccccc3-n3c4ccccc4c4ccccc43)c3oc4ccccc4c3c2)cc1 ; c1ccc(N(c2cccc(-c3ccc4c(ccc5ccccc54)c3)c2)c2cc(-c3ccccc3-n3c4ccccc4c4ccccc43)c3oc4ccccc4c3c2)cc1]

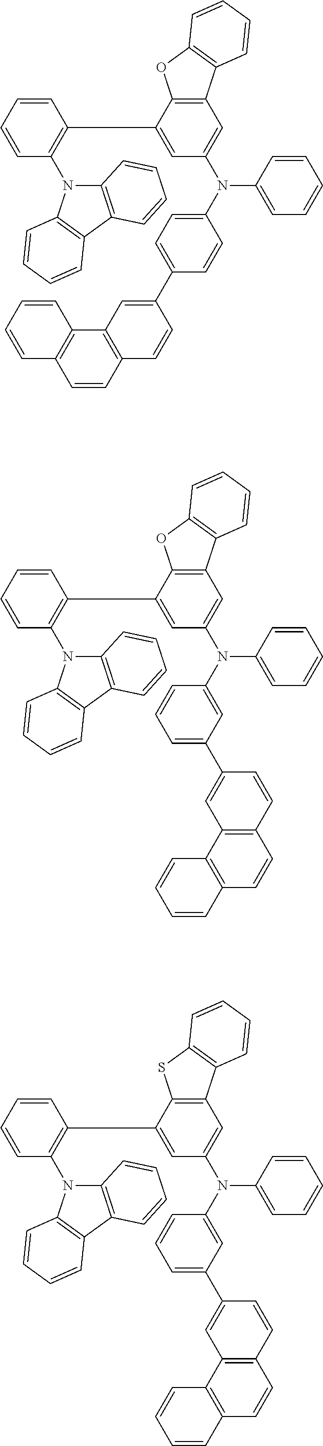 [SMILES: c1ccc(N(c2ccc(-c3ccc4ccc5ccccc5c4c3)cc2)c2cc(-c3ccccc3-n3c4ccccc4c4ccccc43)c3oc4ccccc4c3c2)cc1 ; c1ccc(N(c2cccc(-c3ccc4ccc5ccccc5c4c3)c2)c2cc(-c3ccccc3-n3c4ccccc4c4ccccc43)c3oc4ccccc4c3c2)cc1 ; c1ccc(N(c2cccc(-c3ccc4ccc5ccccc5c4c3)c2)c2cc(-c3ccccc3-n3c4ccccc4c4ccccc43)c3sc4ccccc4c3c2)cc1]

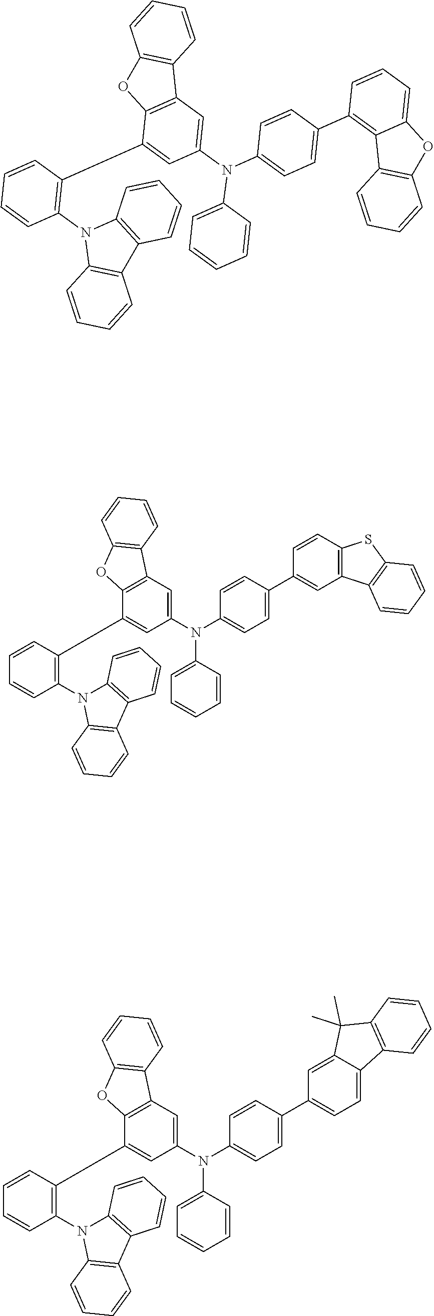 [SMILES: c1ccc(N(c2ccc(-c3cccc4oc5ccccc5c34)cc2)c2cc(-c3ccccc3-n3c4ccccc4c4ccccc43)c3oc4ccccc4c3c2)cc1 ; c1ccc(N(c2ccc(-c3ccc4sc5ccccc5c4c3)cc2)c2cc(-c3ccccc3-n3c4ccccc4c4ccccc43)c3oc4ccccc4c3c2)cc1 ; CC1(C)c2ccccc2-c2ccc(-c3ccc(N(c4ccccc4)c4cc(-c5ccccc5-n5c6ccccc6c6ccccc65)c5oc6ccccc6c5c4)cc3)cc21]

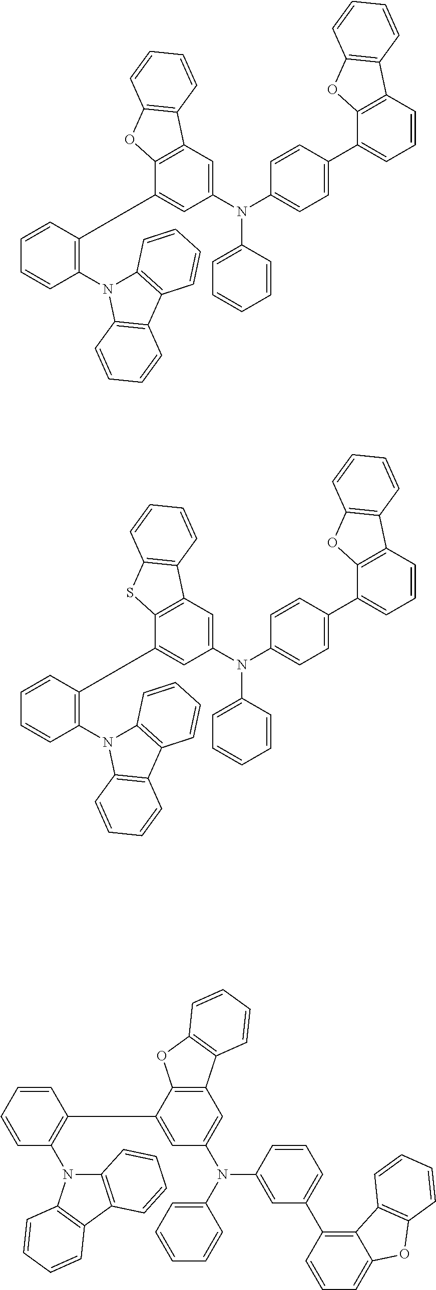 [SMILES: c1ccc(N(c2ccc(-c3cccc4c3oc3ccccc34)cc2)c2cc(-c3ccccc3-n3c4ccccc4c4ccccc43)c3oc4ccccc4c3c2)cc1 ; c1ccc(N(c2ccc(-c3cccc4c3oc3ccccc34)cc2)c2cc(-c3ccccc3-n3c4ccccc4c4ccccc43)c3sc4ccccc4c3c2)cc1 ; c1ccc(N(c2cccc(-c3cccc4oc5ccccc5c34)c2)c2cc(-c3ccccc3-n3c4ccccc4c4ccccc43)c3oc4ccccc4c3c2)cc1]

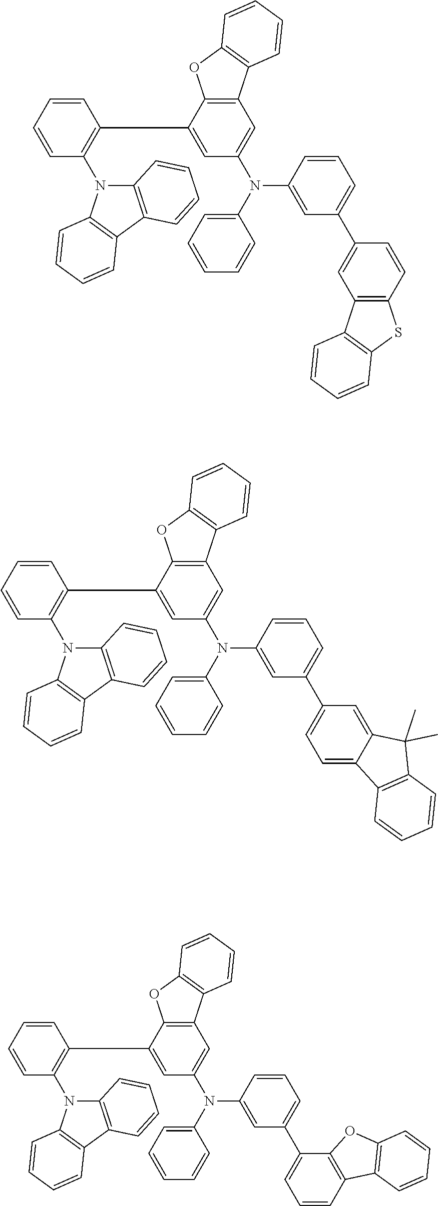 [SMILES: c1ccc(N(c2cccc(-c3ccc4sc5ccccc5c4c3)c2)c2cc(-c3ccccc3-n3c4ccccc4c4ccccc43)c3oc4ccccc4c3c2)cc1 ; CC1(C)c2ccccc2-c2ccc(-c3cccc(N(c4ccccc4)c4cc(-c5ccccc5-n5c6ccccc6c6ccccc65)c5oc6ccccc6c5c4)c3)cc21 ; c1ccc(N(c2cccc(-c3cccc4c3oc3ccccc34)c2)c2cc(-c3ccccc3-n3c4ccccc4c4ccccc43)c3oc4ccccc4c3c2)cc1]

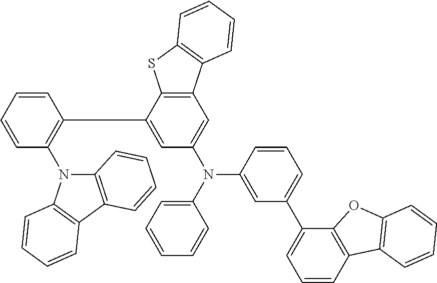 [SMILES: c1ccc(N(c2cccc(-c3cccc4c3oc3ccccc34)c2)c2cc(-c3ccccc3-n3c4ccccc4c4ccccc43)c3sc4ccccc4c3c2)cc1]

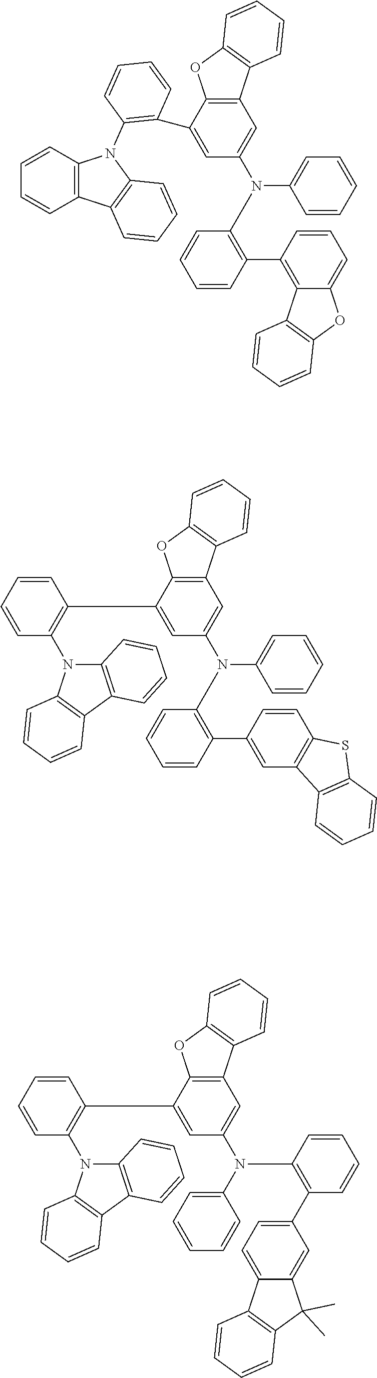 [SMILES: c1ccc(N(c2cc(-c3ccccc3-n3c4ccccc4c4ccccc43)c3oc4ccccc4c3c2)c2ccccc2-c2cccc3oc4ccccc4c23)cc1 ; c1ccc(N(c2cc(-c3ccccc3-n3c4ccccc4c4ccccc43)c3oc4ccccc4c3c2)c2ccccc2-c2ccc3sc4ccccc4c3c2)cc1 ; CC1(C)c2ccccc2-c2ccc(-c3ccccc3N(c3ccccc3)c3cc(-c4ccccc4-n4c5ccccc5c5ccccc54)c4oc5ccccc5c4c3)cc21]

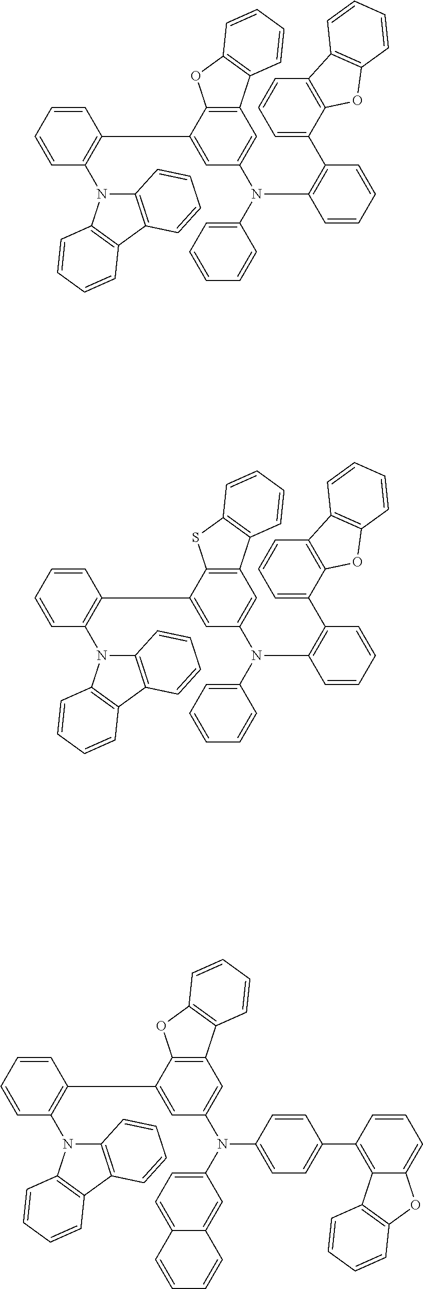 [SMILES: c1ccc(N(c2cc(-c3ccccc3-n3c4ccccc4c4ccccc43)c3oc4ccccc4c3c2)c2ccccc2-c2cccc3c2oc2ccccc23)cc1 ; c1ccc(N(c2cc(-c3ccccc3-n3c4ccccc4c4ccccc43)c3sc4ccccc4c3c2)c2ccccc2-c2cccc3c2oc2ccccc23)cc1 ; c1ccc(-n2c3ccccc3c3ccccc32)c(-c2cc(N(c3ccc(-c4cccc5oc6ccccc6c45)cc3)c3ccc4ccccc4c3)cc3c2oc2ccccc23)c1]

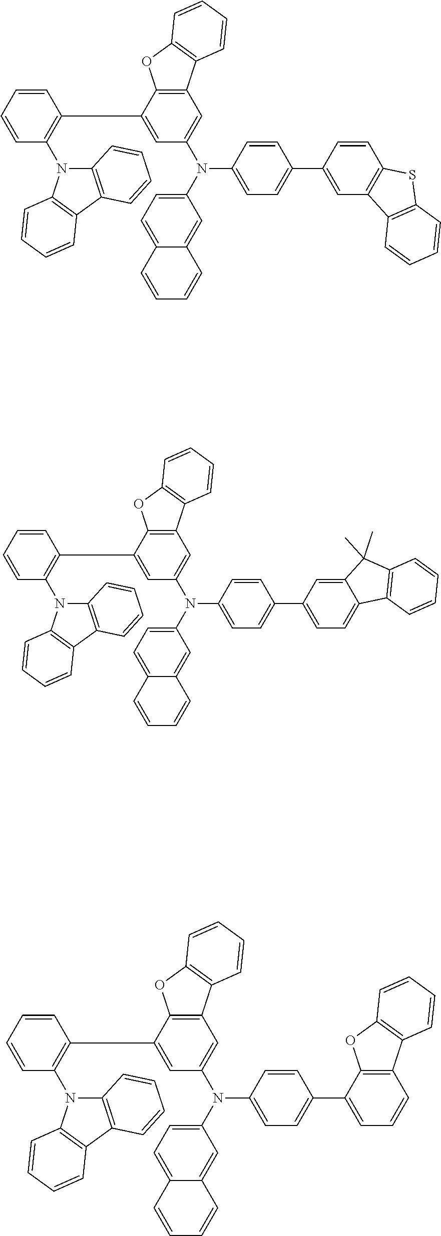 [SMILES: c1ccc(-n2c3ccccc3c3ccccc32)c(-c2cc(N(c3ccc(-c4ccc5sc6ccccc6c5c4)cc3)c3ccc4ccccc4c3)cc3c2oc2ccccc23)c1 ; CC1(C)c2ccccc2-c2ccc(-c3ccc(N(c4ccc5ccccc5c4)c4cc(-c5ccccc5-n5c6ccccc6c6ccccc65)c5oc6ccccc6c5c4)cc3)cc21 ; c1ccc(-n2c3ccccc3c3ccccc32)c(-c2cc(N(c3ccc(-c4cccc5c4oc4ccccc45)cc3)c3ccc4ccccc4c3)cc3c2oc2ccccc23)c1]

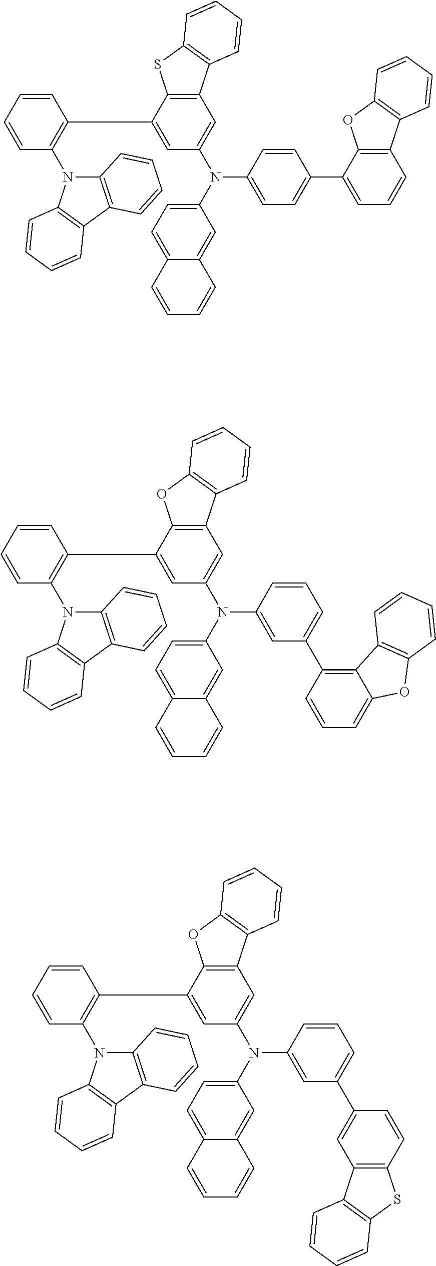 [SMILES: c1ccc(-n2c3ccccc3c3ccccc32)c(-c2cc(N(c3ccc(-c4cccc5c4oc4ccccc45)cc3)c3ccc4ccccc4c3)cc3c2sc2ccccc23)c1 ; c1cc(-c2cccc3oc4ccccc4c23)cc(N(c2ccc3ccccc3c2)c2cc(-c3ccccc3-n3c4ccccc4c4ccccc43)c3oc4ccccc4c3c2)c1 ; c1cc(-c2ccc3sc4ccccc4c3c2)cc(N(c2ccc3ccccc3c2)c2cc(-c3ccccc3-n3c4ccccc4c4ccccc43)c3oc4ccccc4c3c2)c1]

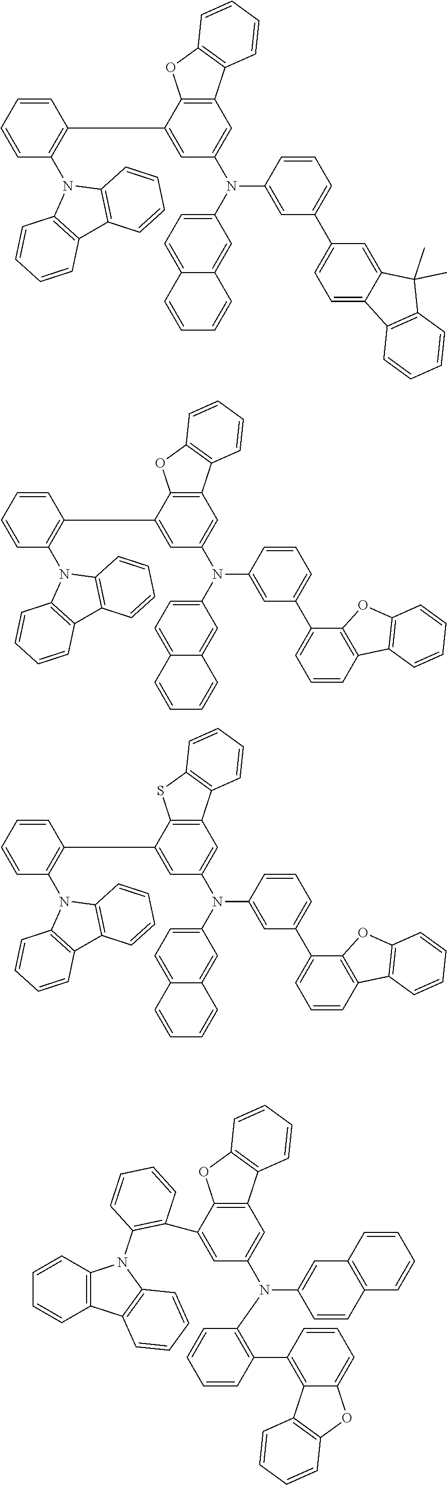

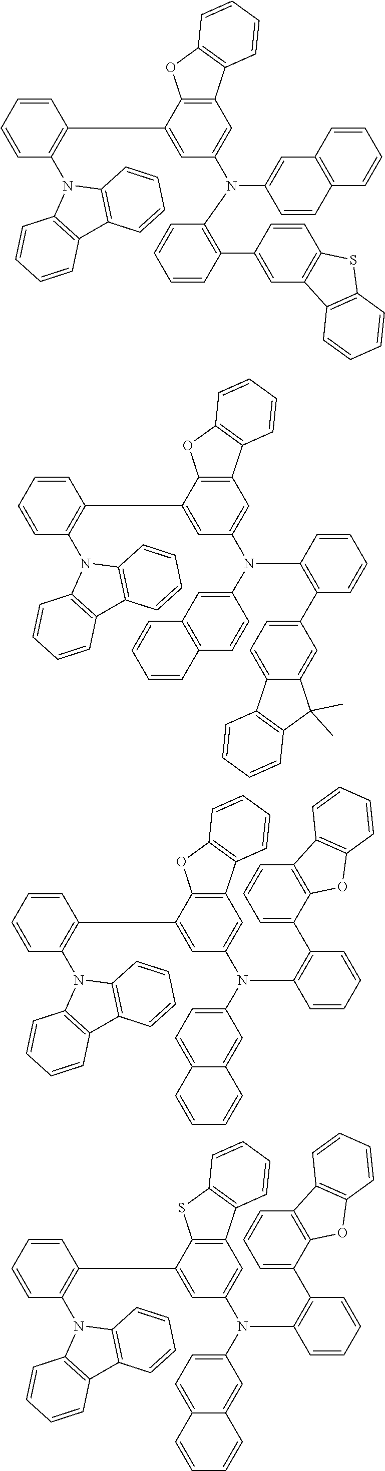

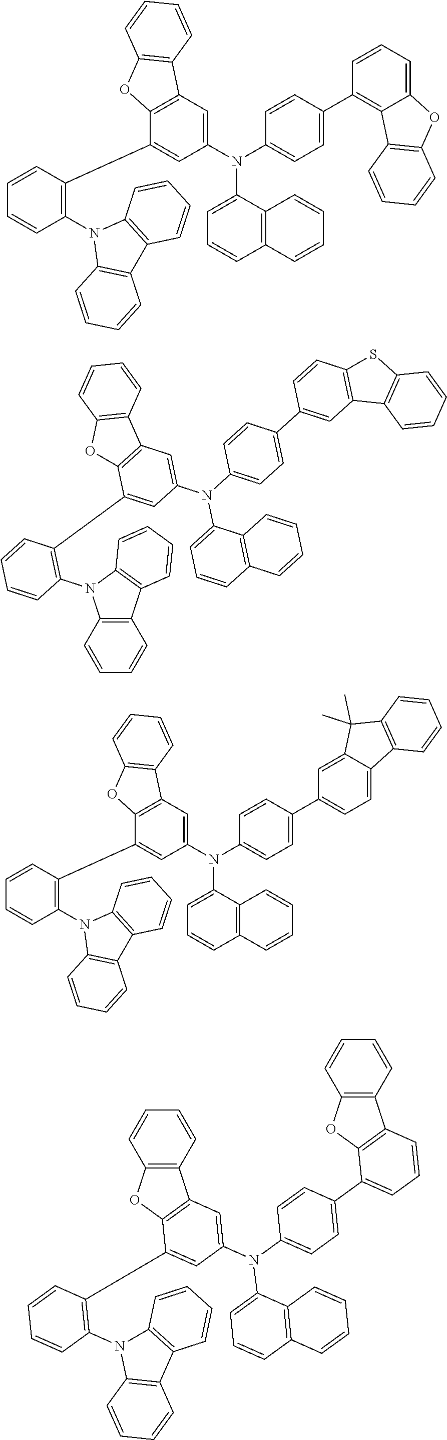

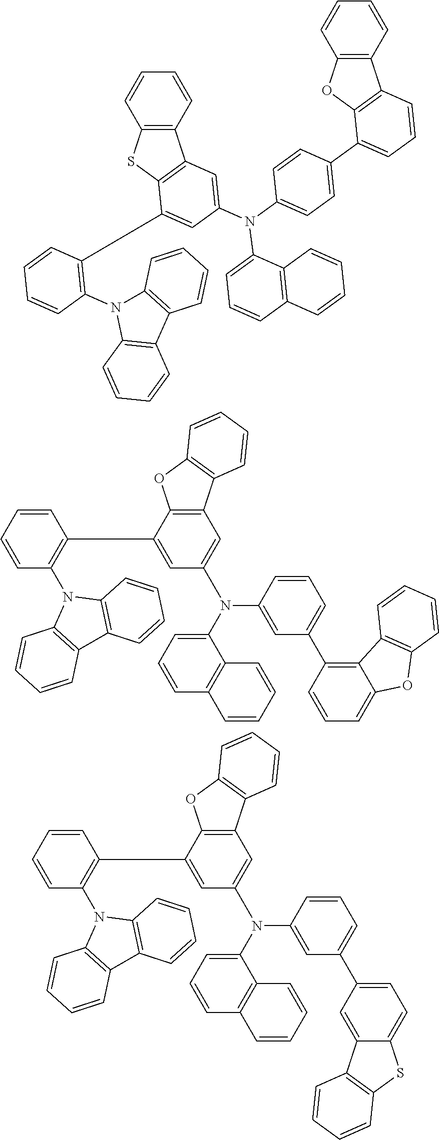

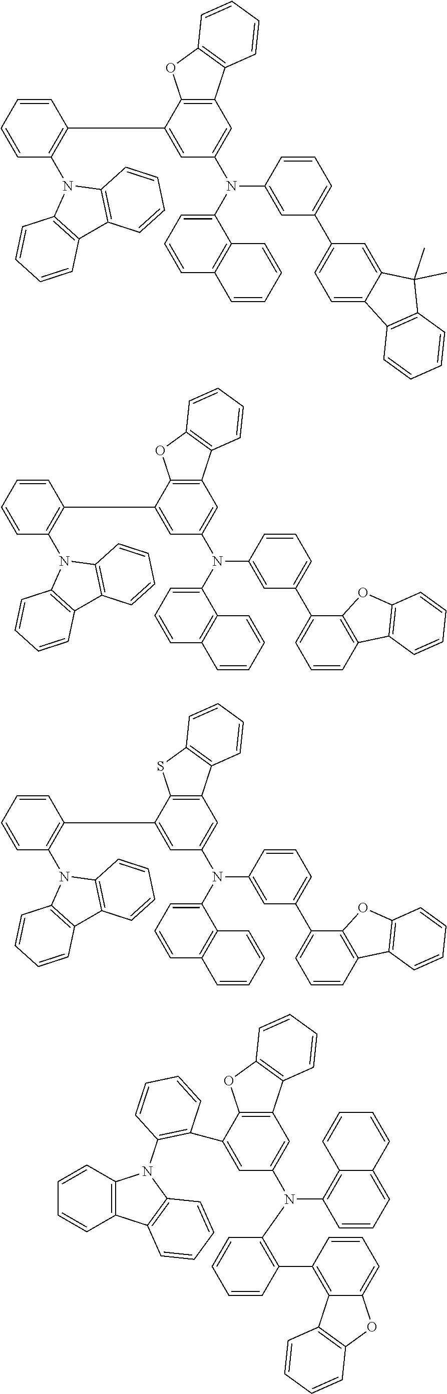

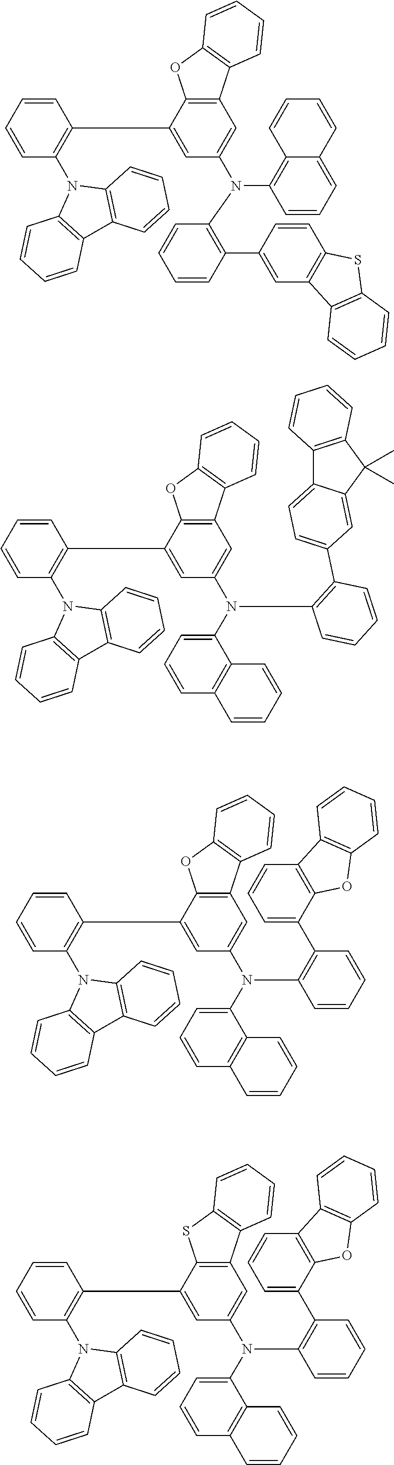

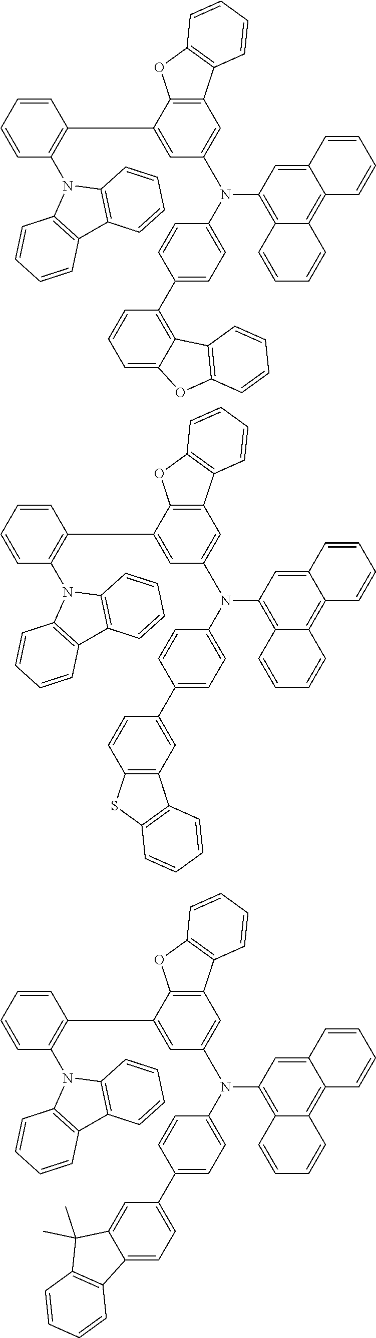

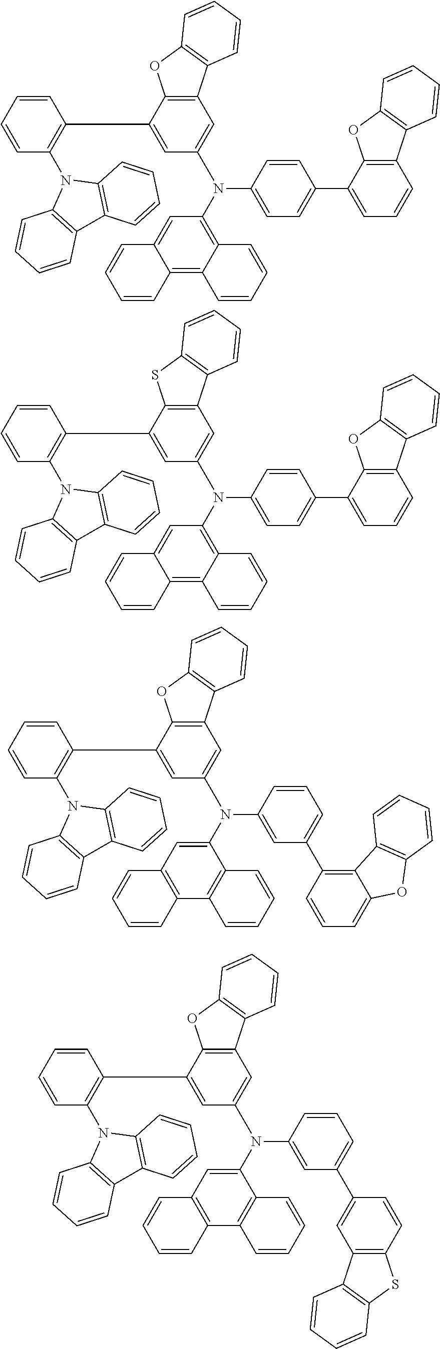

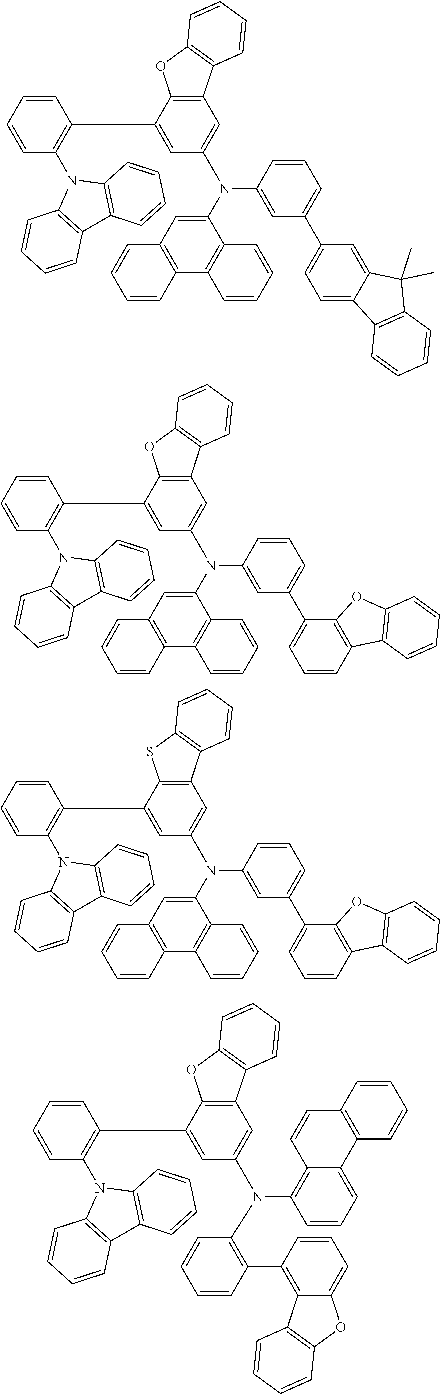

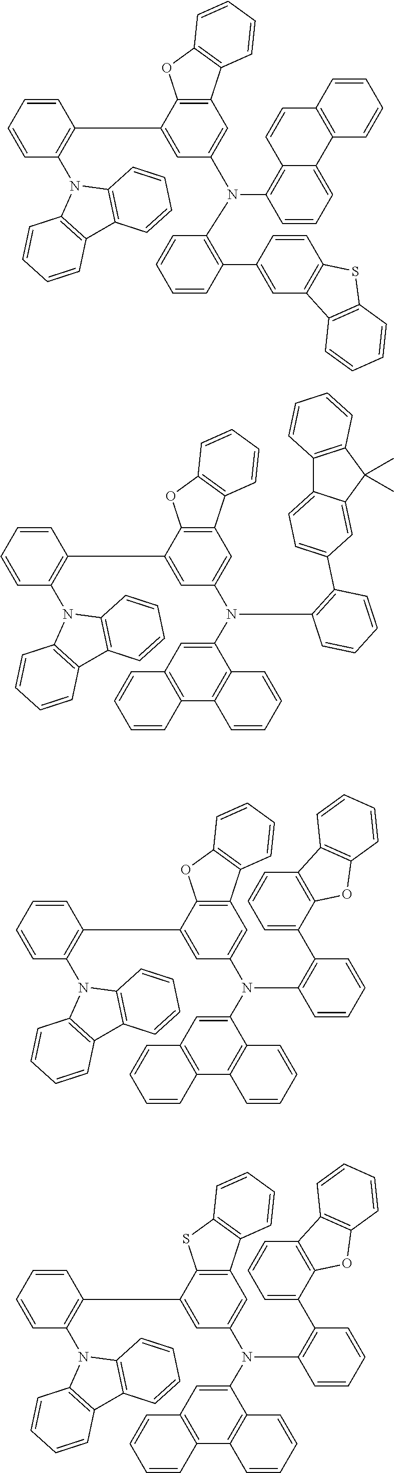

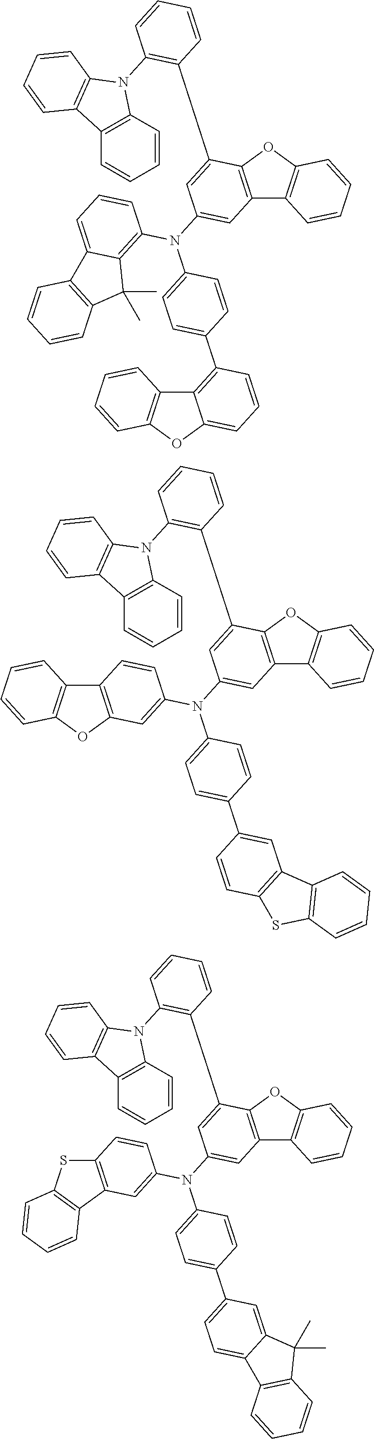

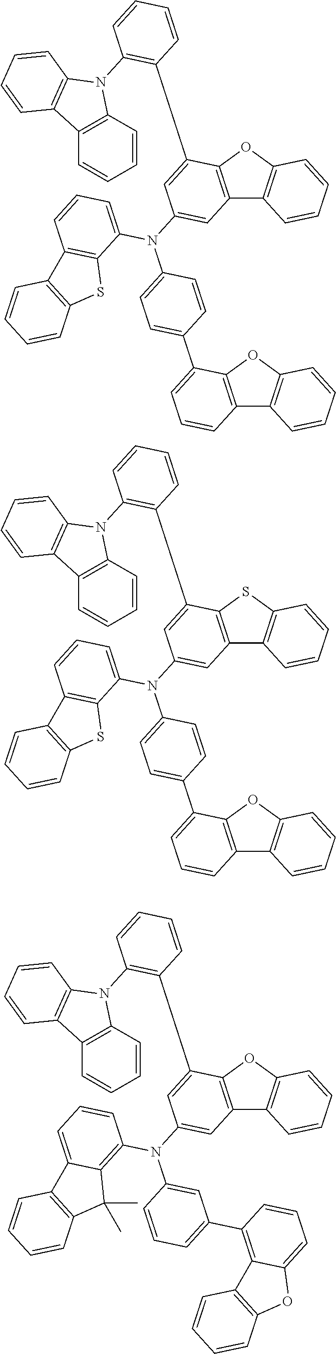

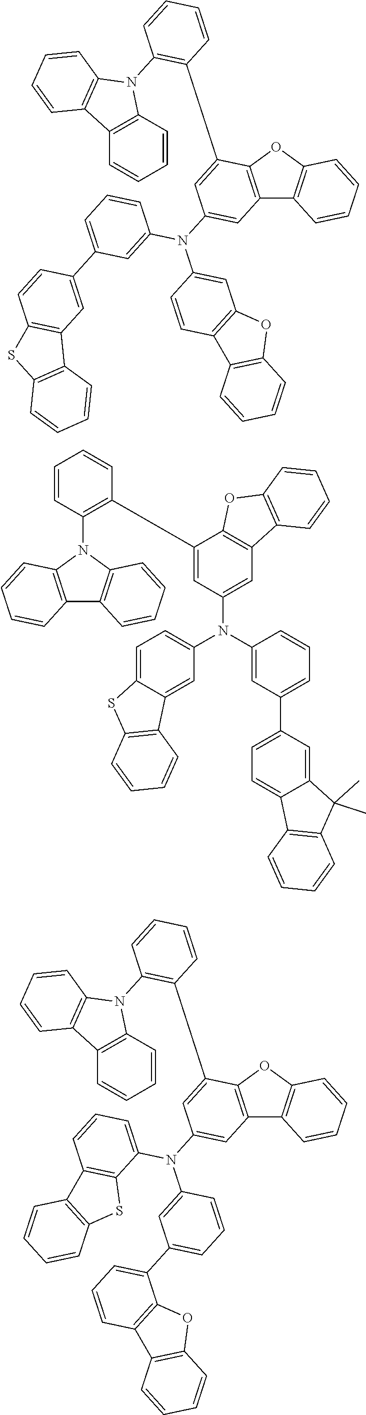

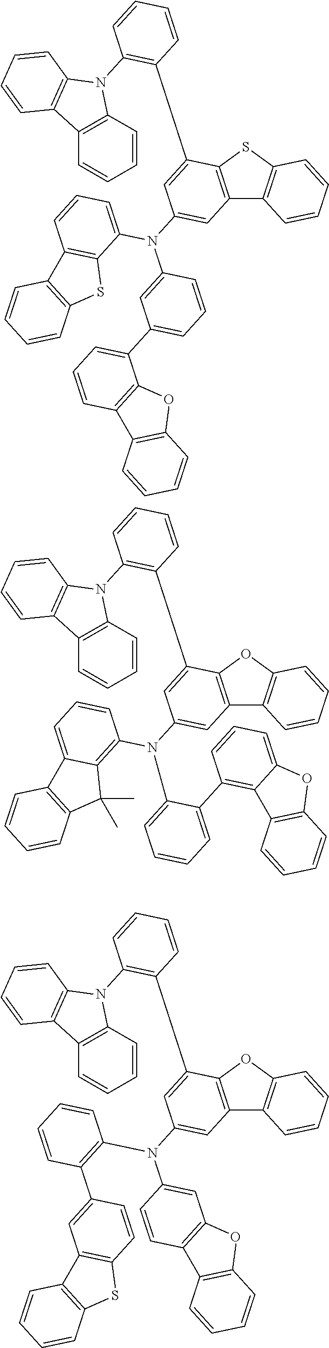

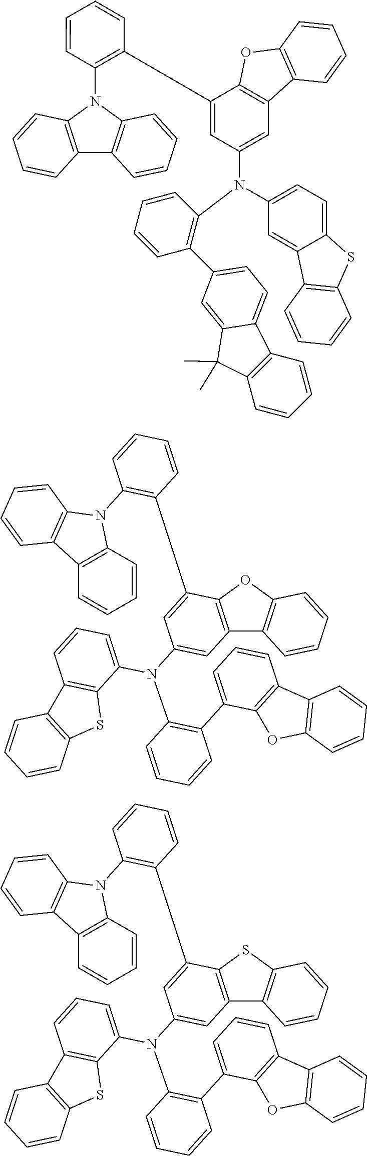

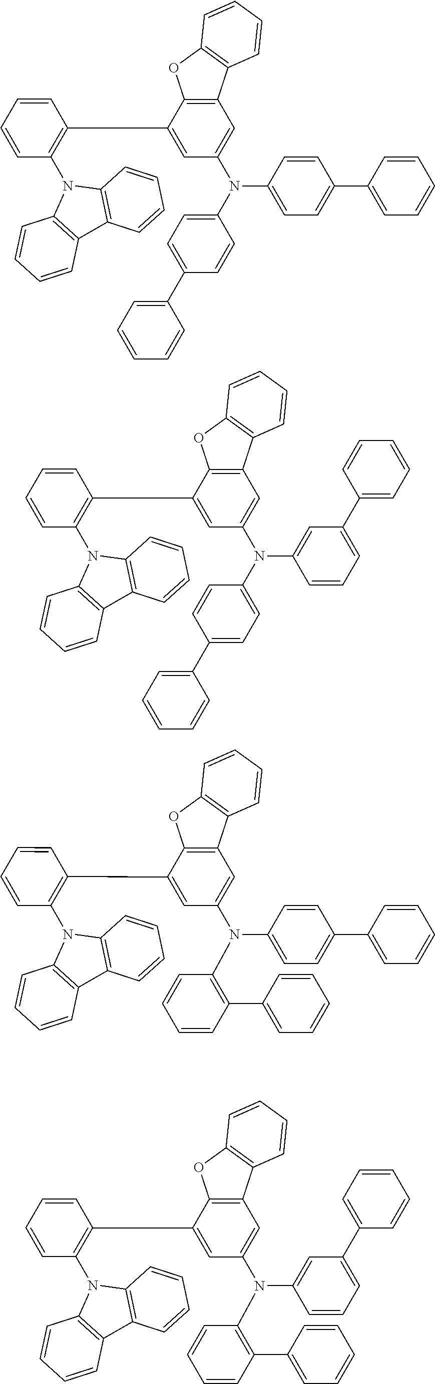

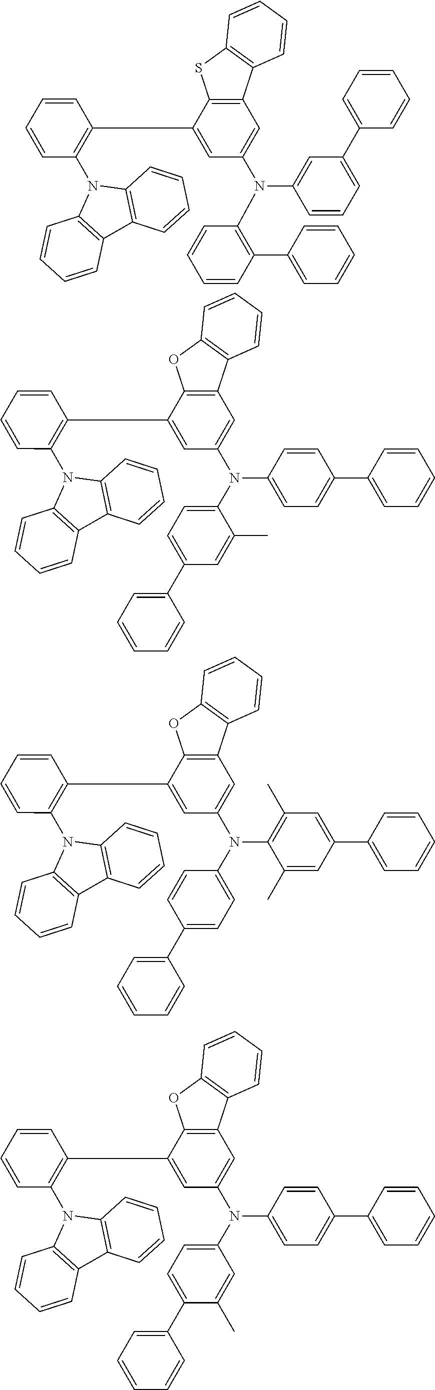

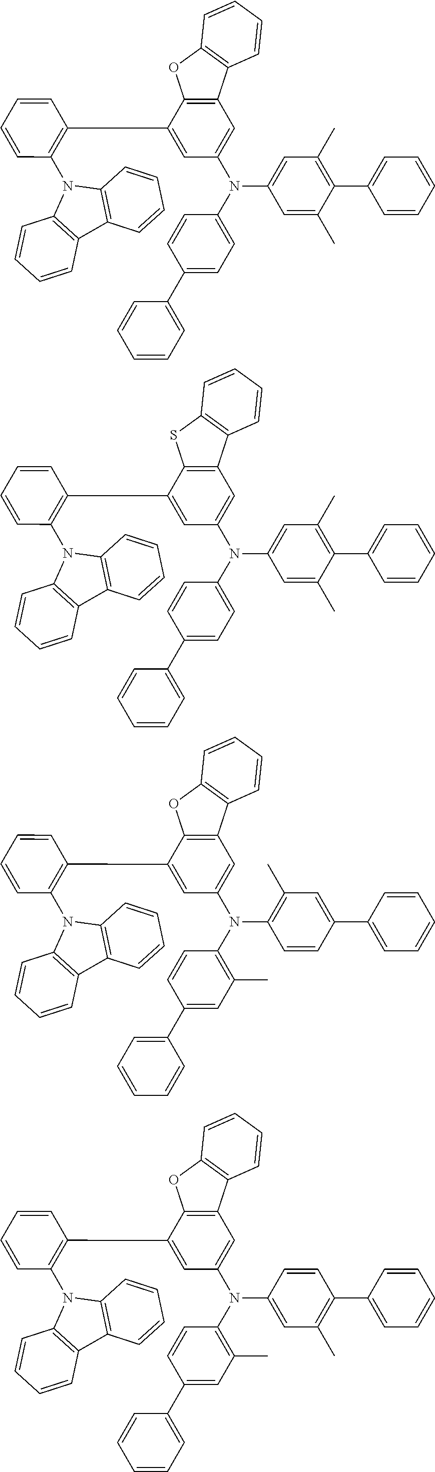

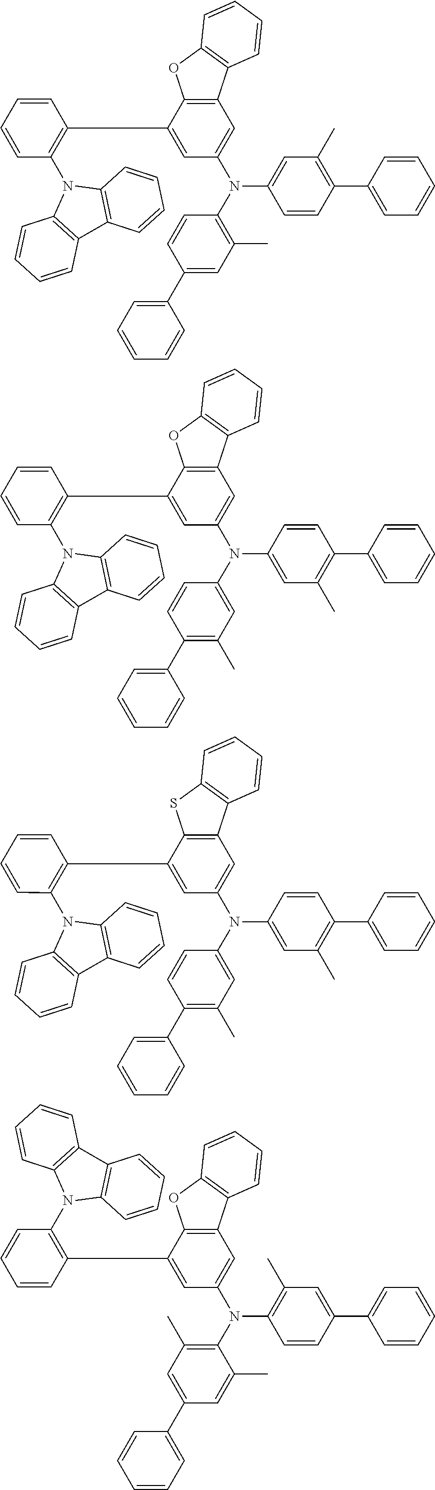

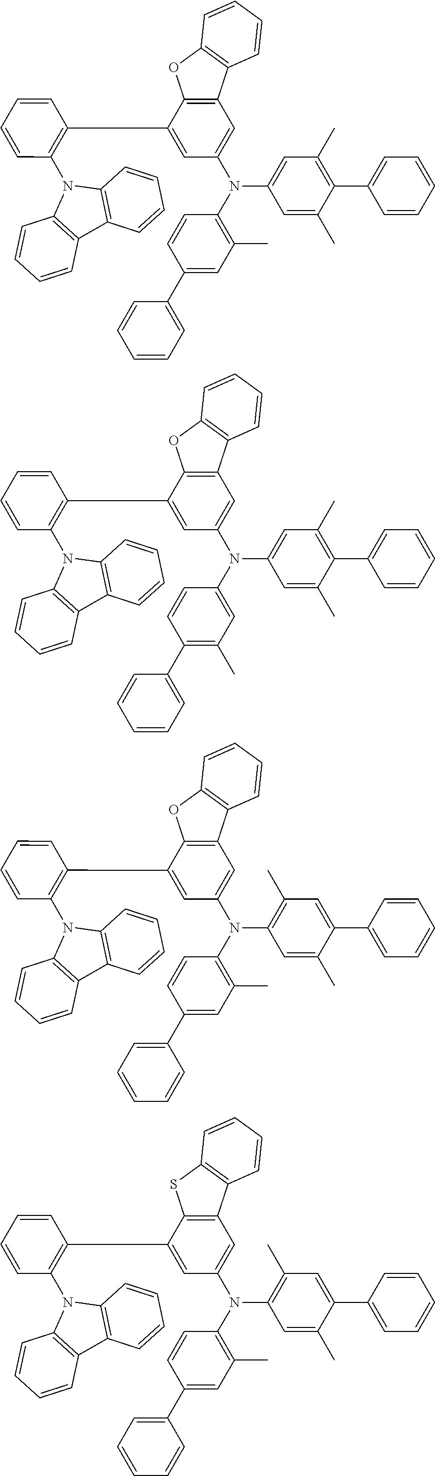

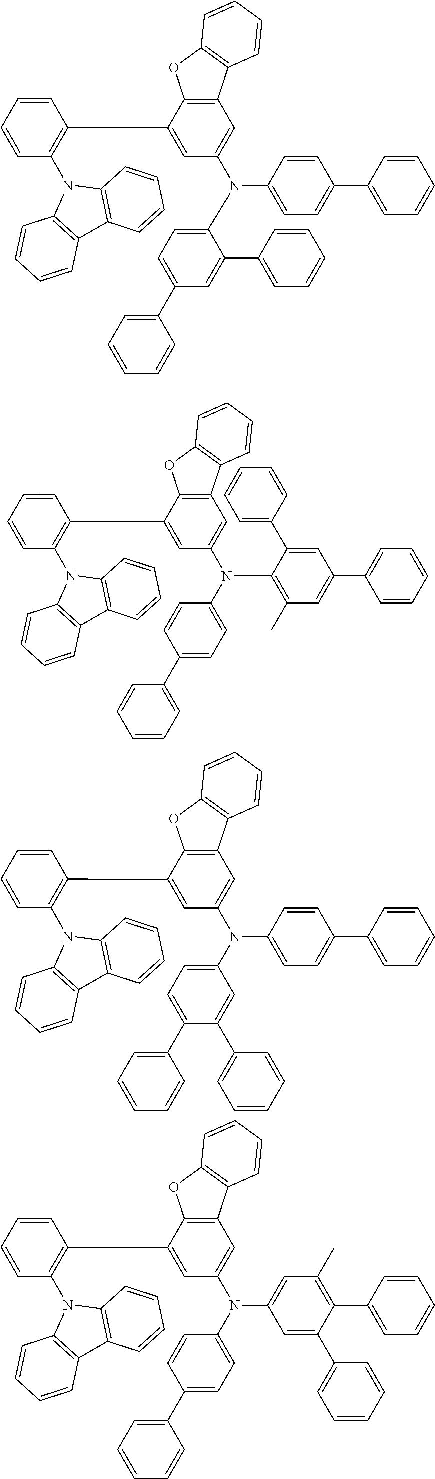

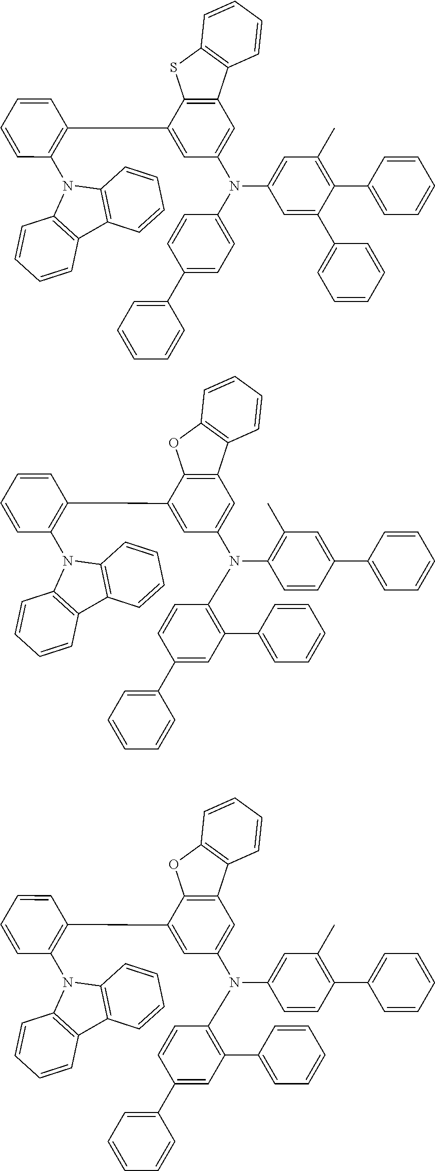 [SMILES: Cc1cc(N(c2ccc(-c3ccccc3)cc2)c2cc(-c3ccccc3-n3c4ccccc4c4ccccc43)c3sc4ccccc4c3c2)cc(-c2ccccc2)c1-c1ccccc1 ; Cc1cc(-c2ccccc2)ccc1N(c1cc(-c2ccccc2-n2c3ccccc3c3ccccc32)c2oc3ccccc3c2c1)c1ccc(-c2ccccc2)cc1-c1ccccc1 ; Cc1cc(N(c2cc(-c3ccccc3-n3c4ccccc4c4ccccc43)c3oc4ccccc4c3c2)c2ccc(-c3ccccc3)cc2-c2ccccc2)ccc1-c1ccccc1]

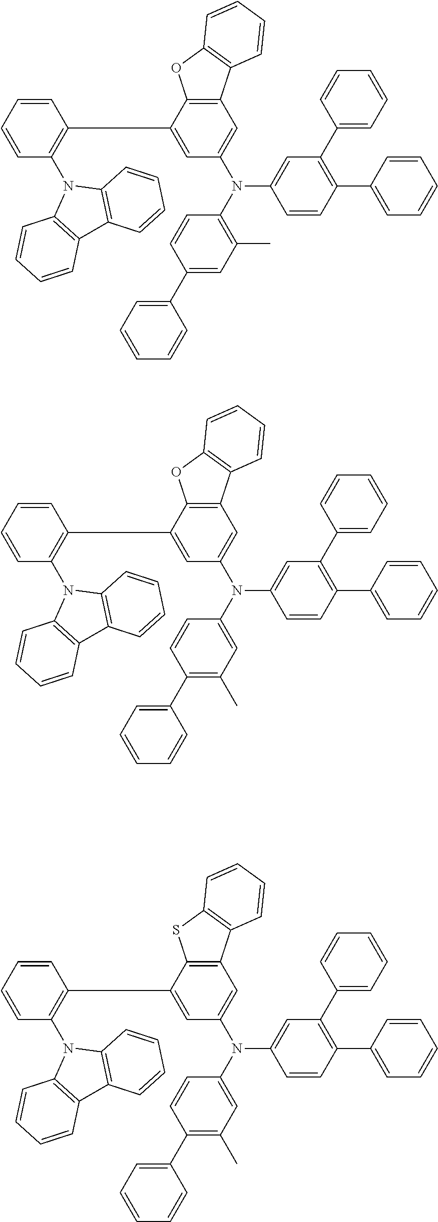 [SMILES: Cc1cc(-c2ccccc2)ccc1N(c1ccc(-c2ccccc2)c(-c2ccccc2)c1)c1cc(-c2ccccc2-n2c3ccccc3c3ccccc32)c2oc3ccccc3c2c1 ; Cc1cc(N(c2ccc(-c3ccccc3)c(-c3ccccc3)c2)c2cc(-c3ccccc3-n3c4ccccc4c4ccccc43)c3oc4ccccc4c3c2)ccc1-c1ccccc1 ; Cc1cc(N(c2ccc(-c3ccccc3)c(-c3ccccc3)c2)c2cc(-c3ccccc3-n3c4ccccc4c4ccccc43)c3sc4ccccc4c3c2)ccc1-c1ccccc1]

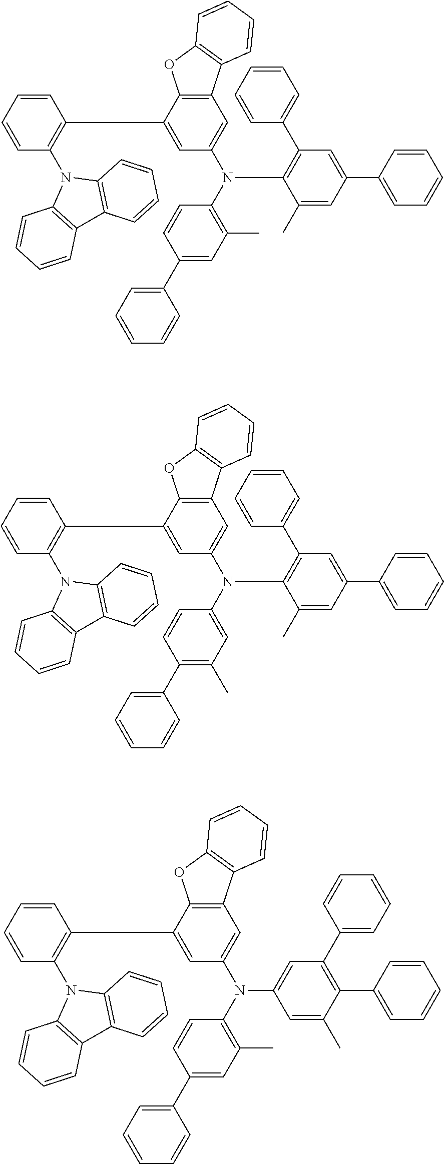 [SMILES: Cc1cc(-c2ccccc2)ccc1N(c1cc(-c2ccccc2-n2c3ccccc3c3ccccc32)c2oc3ccccc3c2c1)c1c(C)cc(-c2ccccc2)cc1-c1ccccc1 ; Cc1cc(N(c2cc(-c3ccccc3-n3c4ccccc4c4ccccc43)c3oc4ccccc4c3c2)c2c(C)cc(-c3ccccc3)cc2-c2ccccc2)ccc1-c1ccccc1 ; Cc1cc(-c2ccccc2)ccc1N(c1cc(C)c(-c2ccccc2)c(-c2ccccc2)c1)c1cc(-c2ccccc2-n2c3ccccc3c3ccccc32)c2oc3ccccc3c2c1]

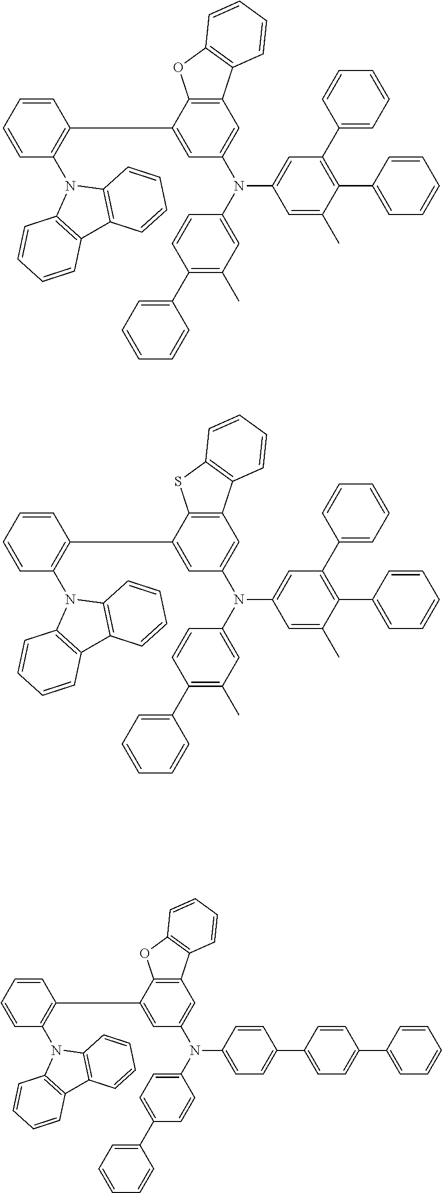 [SMILES: Cc1cc(N(c2cc(C)c(-c3ccccc3)c(-c3ccccc3)c2)c2cc(-c3ccccc3-n3c4ccccc4c4ccccc43)c3oc4ccccc4c3c2)ccc1-c1ccccc1 ; Cc1cc(N(c2cc(C)c(-c3ccccc3)c(-c3ccccc3)c2)c2cc(-c3ccccc3-n3c4ccccc4c4ccccc43)c3sc4ccccc4c3c2)ccc1-c1ccccc1 ; c1ccc(-c2ccc(-c3ccc(N(c4ccc(-c5ccccc5)cc4)c4cc(-c5ccccc5-n5c6ccccc6c6ccccc65)c5oc6ccccc6c5c4)cc3)cc2)cc1]

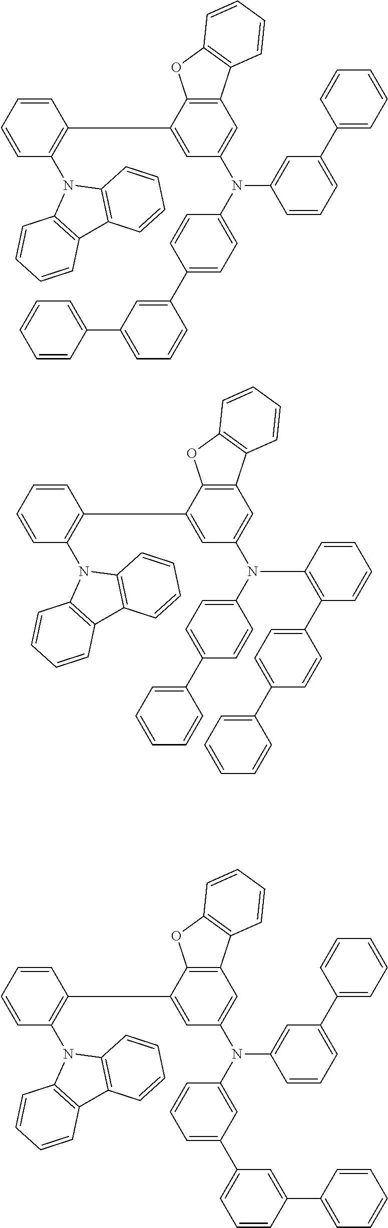 [SMILES: c1ccc(-c2cccc(-c3ccc(N(c4cccc(-c5ccccc5)c4)c4cc(-c5ccccc5-n5c6ccccc6c6ccccc65)c5oc6ccccc6c5c4)cc3)c2)cc1 ; c1ccc(-c2ccc(-c3ccccc3N(c3ccc(-c4ccccc4)cc3)c3cc(-c4ccccc4-n4c5ccccc5c5ccccc54)c4oc5ccccc5c4c3)cc2)cc1 ; c1ccc(-c2cccc(-c3cccc(N(c4cccc(-c5ccccc5)c4)c4cc(-c5ccccc5-n5c6ccccc6c6ccccc65)c5oc6ccccc6c5c4)c3)c2)cc1]

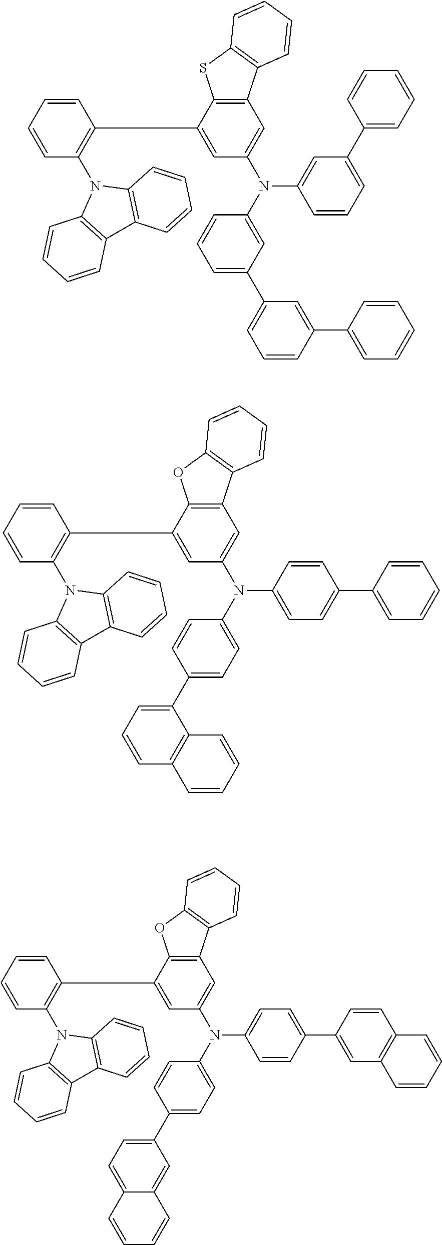 [SMILES: c1ccc(-c2cccc(-c3cccc(N(c4cccc(-c5ccccc5)c4)c4cc(-c5ccccc5-n5c6ccccc6c6ccccc65)c5sc6ccccc6c5c4)c3)c2)cc1 ; c1ccc(-c2ccc(N(c3ccc(-c4cccc5ccccc45)cc3)c3cc(-c4ccccc4-n4c5ccccc5c5ccccc54)c4oc5ccccc5c4c3)cc2)cc1 ; c1ccc(-n2c3ccccc3c3ccccc32)c(-c2cc(N(c3ccc(-c4ccc5ccccc5c4)cc3)c3ccc(-c4ccc5ccccc5c4)cc3)cc3c2oc2ccccc23)c1]

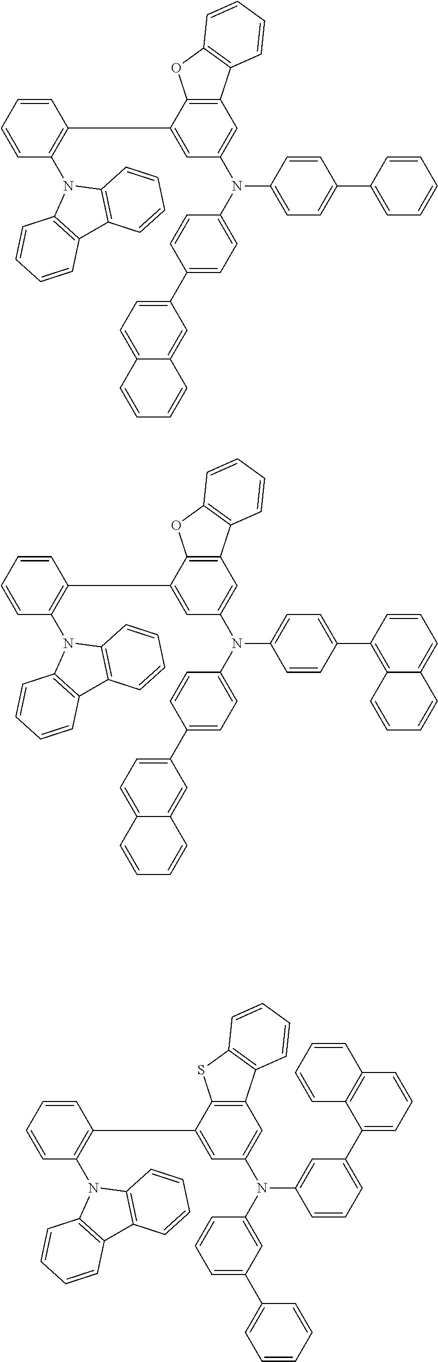 [SMILES: c1ccc(-c2ccc(N(c3ccc(-c4ccc5ccccc5c4)cc3)c3cc(-c4ccccc4-n4c5ccccc5c5ccccc54)c4oc5ccccc5c4c3)cc2)cc1 ; c1ccc(-n2c3ccccc3c3ccccc32)c(-c2cc(N(c3ccc(-c4ccc5ccccc5c4)cc3)c3ccc(-c4cccc5ccccc45)cc3)cc3c2oc2ccccc23)c1 ; c1ccc(-c2cccc(N(c3cccc(-c4cccc5ccccc45)c3)c3cc(-c4ccccc4-n4c5ccccc5c5ccccc54)c4sc5ccccc5c4c3)c2)cc1]

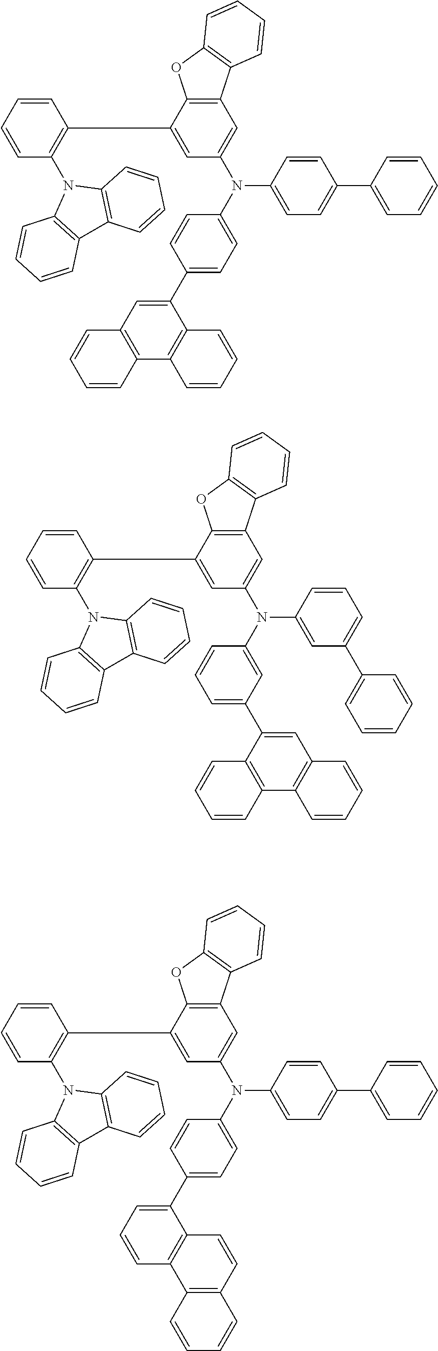 [SMILES: c1ccc(-c2ccc(N(c3ccc(-c4cc5ccccc5c5ccccc45)cc3)c3cc(-c4ccccc4-n4c5ccccc5c5ccccc54)c4oc5ccccc5c4c3)cc2)cc1 ; c1ccc(-c2cccc(N(c3cccc(-c4cc5ccccc5c5ccccc45)c3)c3cc(-c4ccccc4-n4c5ccccc5c5ccccc54)c4oc5ccccc5c4c3)c2)cc1 ; c1ccc(-c2ccc(N(c3ccc(-c4cccc5c4ccc4ccccc45)cc3)c3cc(-c4ccccc4-n4c5ccccc5c5ccccc54)c4oc5ccccc5c4c3)cc2)cc1]

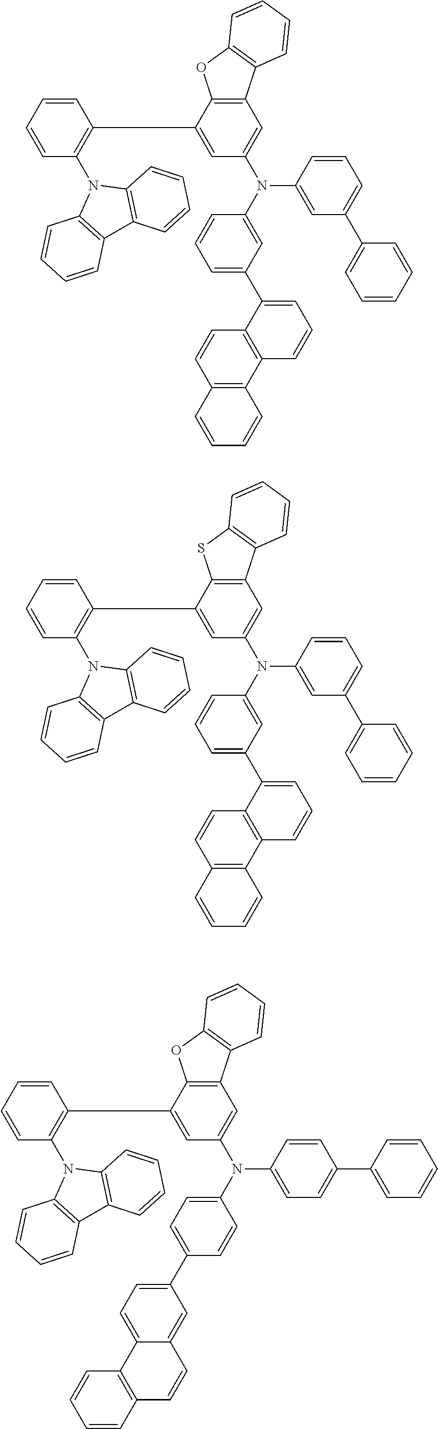

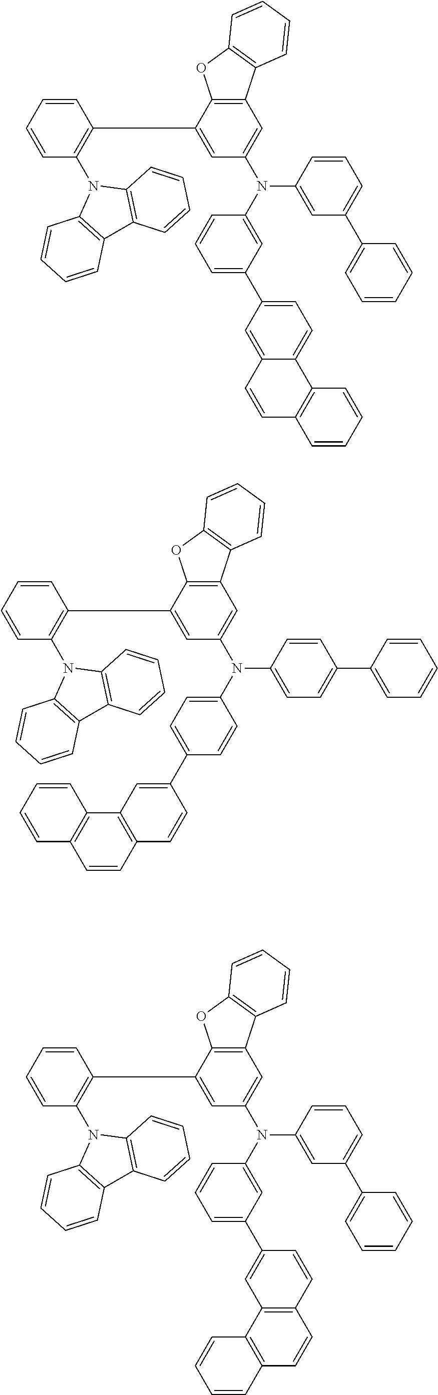 [SMILES: c1ccc(-c2cccc(N(c3cccc(-c4ccc5c(ccc6ccccc65)c4)c3)c3cc(-c4ccccc4-n4c5ccccc5c5ccccc54)c4oc5ccccc5c4c3)c2)cc1 ; c1ccc(-c2ccc(N(c3ccc(-c4ccc5ccc6ccccc6c5c4)cc3)c3cc(-c4ccccc4-n4c5ccccc5c5ccccc54)c4oc5ccccc5c4c3)cc2)cc1 ; c1ccc(-c2cccc(N(c3cccc(-c4ccc5ccc6ccccc6c5c4)c3)c3cc(-c4ccccc4-n4c5ccccc5c5ccccc54)c4oc5ccccc5c4c3)c2)cc1]

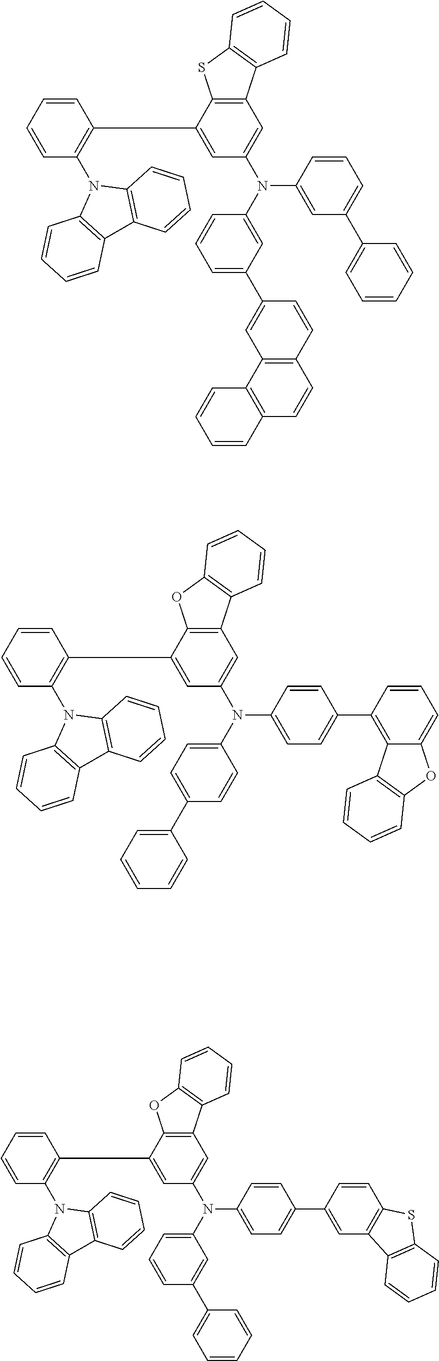 [SMILES: c1ccc(-c2cccc(N(c3cccc(-c4ccc5ccc6ccccc6c5c4)c3)c3cc(-c4ccccc4-n4c5ccccc5c5ccccc54)c4sc5ccccc5c4c3)c2)cc1 ; c1ccc(-c2ccc(N(c3ccc(-c4cccc5oc6ccccc6c45)cc3)c3cc(-c4ccccc4-n4c5ccccc5c5ccccc54)c4oc5ccccc5c4c3)cc2)cc1 ; c1ccc(-c2cccc(N(c3ccc(-c4ccc5sc6ccccc6c5c4)cc3)c3cc(-c4ccccc4-n4c5ccccc5c5ccccc54)c4oc5ccccc5c4c3)c2)cc1]

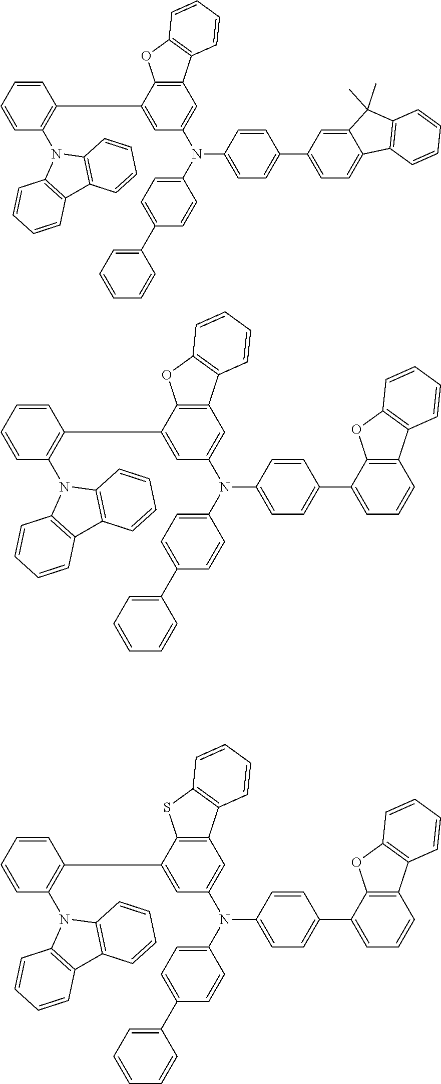 [SMILES: CC1(C)c2ccccc2-c2ccc(-c3ccc(N(c4ccc(-c5ccccc5)cc4)c4cc(-c5ccccc5-n5c6ccccc6c6ccccc65)c5oc6ccccc6c5c4)cc3)cc21 ; c1ccc(-c2ccc(N(c3ccc(-c4cccc5c4oc4ccccc45)cc3)c3cc(-c4ccccc4-n4c5ccccc5c5ccccc54)c4oc5ccccc5c4c3)cc2)cc1 ; c1ccc(-c2ccc(N(c3ccc(-c4cccc5c4oc4ccccc45)cc3)c3cc(-c4ccccc4-n4c5ccccc5c5ccccc54)c4sc5ccccc5c4c3)cc2)cc1]

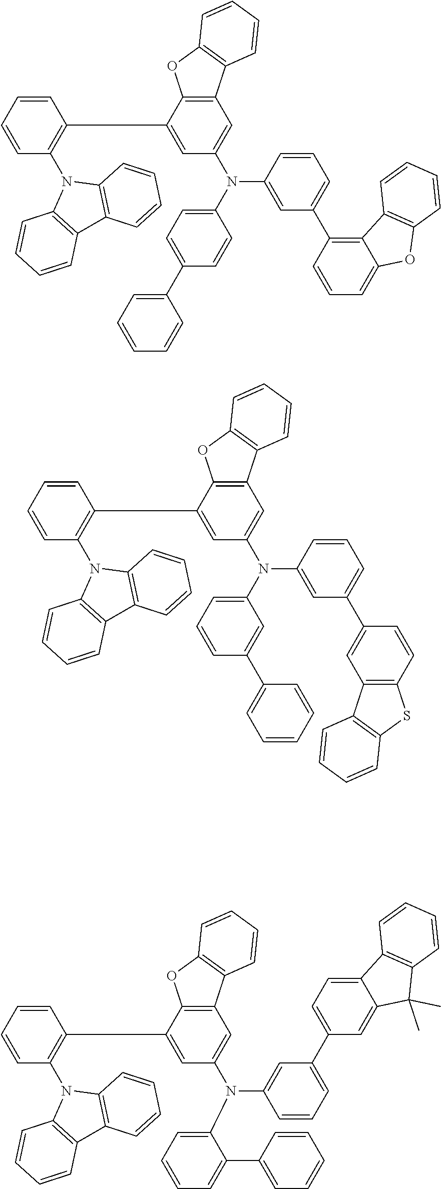 [SMILES: c1ccc(-c2ccc(N(c3cccc(-c4cccc5oc6ccccc6c45)c3)c3cc(-c4ccccc4-n4c5ccccc5c5ccccc54)c4oc5ccccc5c4c3)cc2)cc1 ; c1ccc(-c2cccc(N(c3cccc(-c4ccc5sc6ccccc6c5c4)c3)c3cc(-c4ccccc4-n4c5ccccc5c5ccccc54)c4oc5ccccc5c4c3)c2)cc1 ; CC1(C)c2ccccc2-c2ccc(-c3cccc(N(c4cc(-c5ccccc5-n5c6ccccc6c6ccccc65)c5oc6ccccc6c5c4)c4ccccc4-c4ccccc4)c3)cc21]

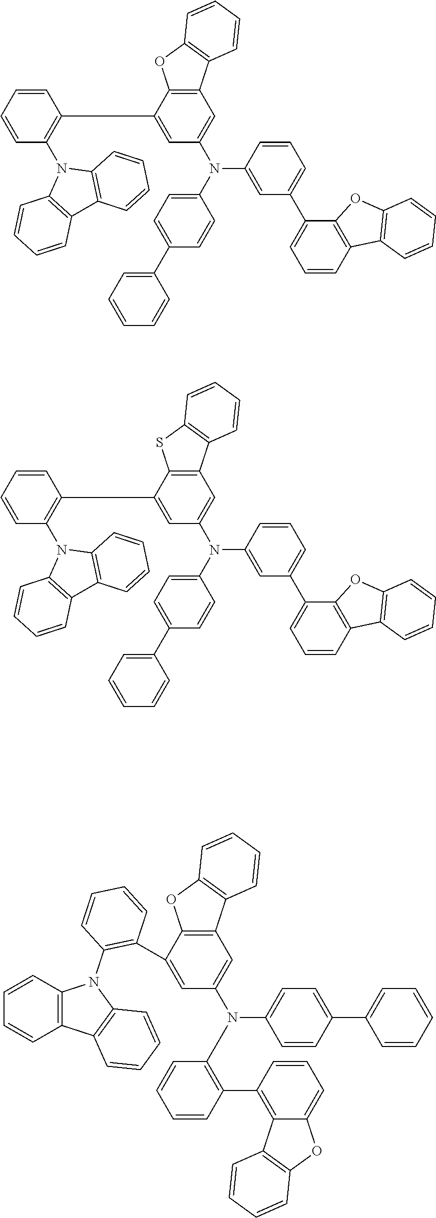 [SMILES: c1ccc(-c2ccc(N(c3cccc(-c4cccc5c4oc4ccccc45)c3)c3cc(-c4ccccc4-n4c5ccccc5c5ccccc54)c4oc5ccccc5c4c3)cc2)cc1 ; c1ccc(-c2ccc(N(c3cccc(-c4cccc5c4oc4ccccc45)c3)c3cc(-c4ccccc4-n4c5ccccc5c5ccccc54)c4sc5ccccc5c4c3)cc2)cc1 ; c1ccc(-c2ccc(N(c3cc(-c4ccccc4-n4c5ccccc5c5ccccc54)c4oc5ccccc5c4c3)c3ccccc3-c3cccc4oc5ccccc5c34)cc2)cc1]

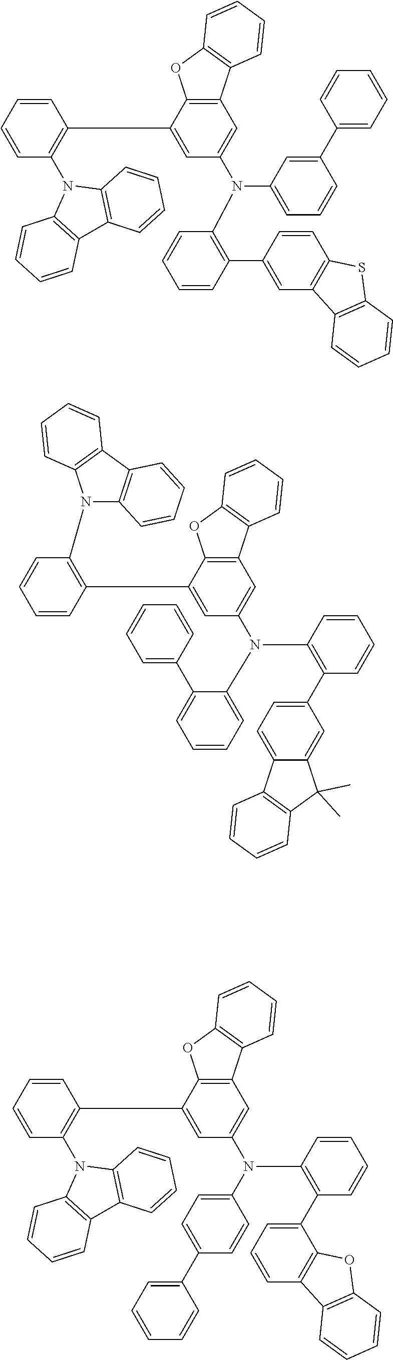 [SMILES: c1ccc(-c2cccc(N(c3cc(-c4ccccc4-n4c5ccccc5c5ccccc54)c4oc5ccccc5c4c3)c3ccccc3-c3ccc4sc5ccccc5c4c3)c2)cc1 ; CC1(C)c2ccccc2-c2ccc(-c3ccccc3N(c3cc(-c4ccccc4-n4c5ccccc5c5ccccc54)c4oc5ccccc5c4c3)c3ccccc3-c3ccccc3)cc21 ; c1ccc(-c2ccc(N(c3cc(-c4ccccc4-n4c5ccccc5c5ccccc54)c4oc5ccccc5c4c3)c3ccccc3-c3cccc4c3oc3ccccc34)cc2)cc1]

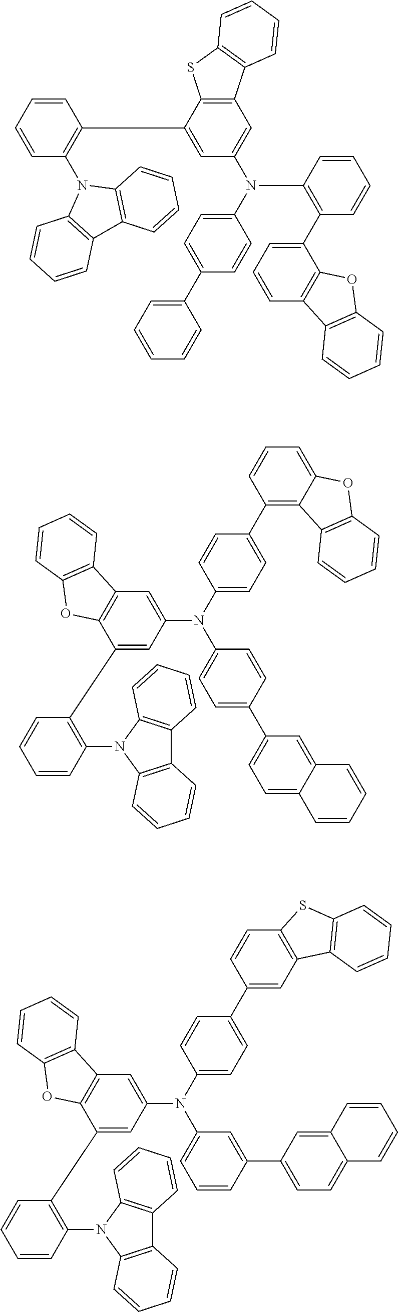 [SMILES: c1ccc(-c2ccc(N(c3cc(-c4ccccc4-n4c5ccccc5c5ccccc54)c4sc5ccccc5c4c3)c3ccccc3-c3cccc4c3oc3ccccc34)cc2)cc1 ; c1ccc(-n2c3ccccc3c3ccccc32)c(-c2cc(N(c3ccc(-c4ccc5ccccc5c4)cc3)c3ccc(-c4cccc5oc6ccccc6c45)cc3)cc3c2oc2ccccc23)c1 ; c1cc(-c2ccc3ccccc3c2)cc(N(c2ccc(-c3ccc4sc5ccccc5c4c3)cc2)c2cc(-c3ccccc3-n3c4ccccc4c4ccccc43)c3oc4ccccc4c3c2)c1]

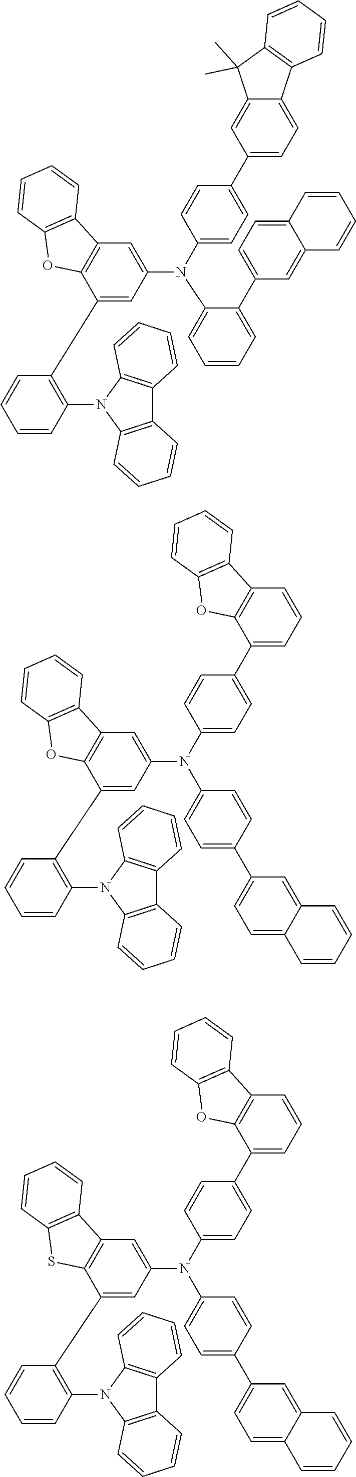 [SMILES: CC1(C)c2ccccc2-c2ccc(-c3ccc(N(c4cc(-c5ccccc5-n5c6ccccc6c6ccccc65)c5oc6ccccc6c5c4)c4ccccc4-c4ccc5ccccc5c4)cc3)cc21 ; c1ccc(-n2c3ccccc3c3ccccc32)c(-c2cc(N(c3ccc(-c4ccc5ccccc5c4)cc3)c3ccc(-c4cccc5c4oc4ccccc45)cc3)cc3c2oc2ccccc23)c1 ; c1ccc(-n2c3ccccc3c3ccccc32)c(-c2cc(N(c3ccc(-c4ccc5ccccc5c4)cc3)c3ccc(-c4cccc5c4oc4ccccc45)cc3)cc3c2sc2ccccc23)c1]

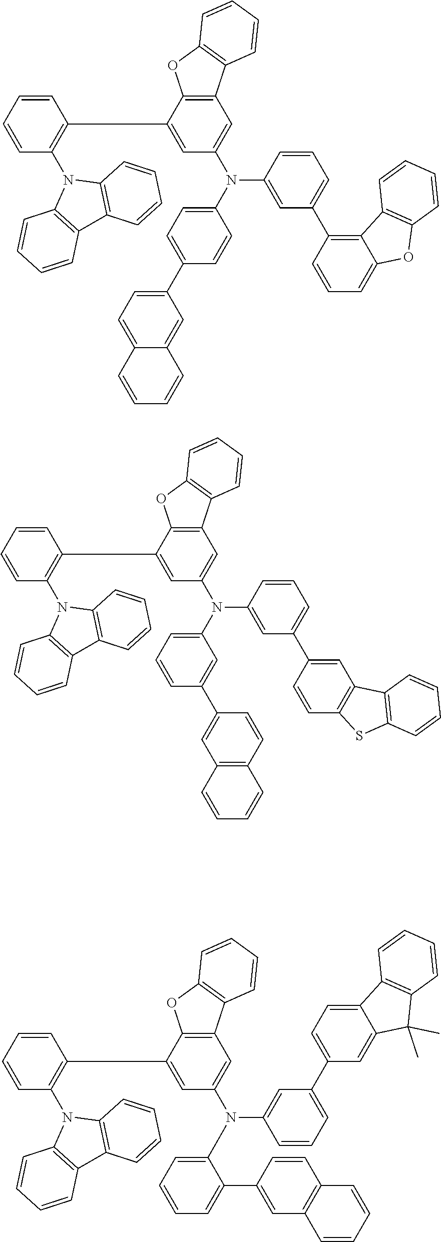 [SMILES: c1cc(-c2cccc3oc4ccccc4c23)cc(N(c2ccc(-c3ccc4ccccc4c3)cc2)c2cc(-c3ccccc3-n3c4ccccc4c4ccccc43)c3oc4ccccc4c3c2)c1 ; c1cc(-c2ccc3ccccc3c2)cc(N(c2cccc(-c3ccc4sc5ccccc5c4c3)c2)c2cc(-c3ccccc3-n3c4ccccc4c4ccccc43)c3oc4ccccc4c3c2)c1 ; CC1(C)c2ccccc2-c2ccc(-c3cccc(N(c4cc(-c5ccccc5-n5c6ccccc6c6ccccc65)c5oc6ccccc6c5c4)c4ccccc4-c4ccc5ccccc5c4)c3)cc21]

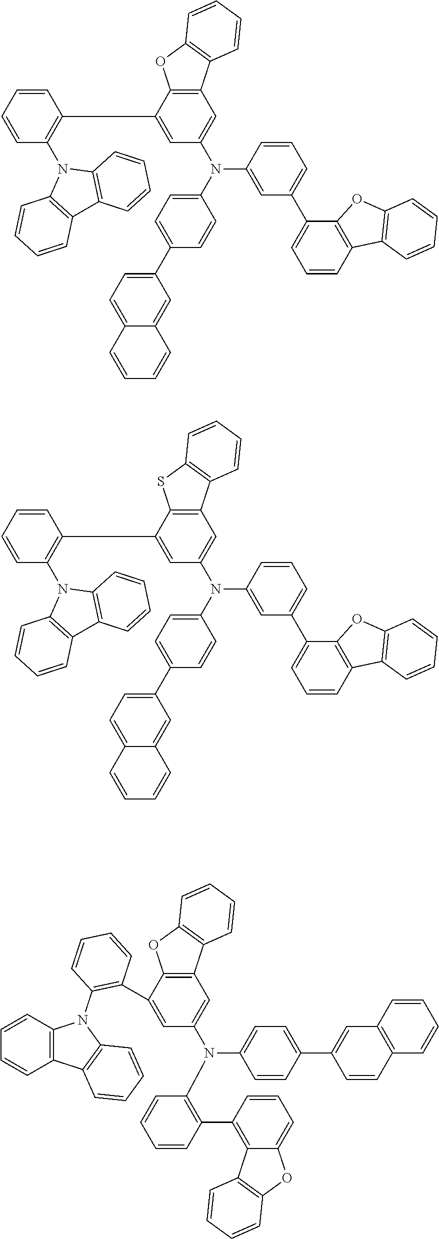 [SMILES: c1cc(-c2cccc3c2oc2ccccc23)cc(N(c2ccc(-c3ccc4ccccc4c3)cc2)c2cc(-c3ccccc3-n3c4ccccc4c4ccccc43)c3oc4ccccc4c3c2)c1 ; c1cc(-c2cccc3c2oc2ccccc23)cc(N(c2ccc(-c3ccc4ccccc4c3)cc2)c2cc(-c3ccccc3-n3c4ccccc4c4ccccc43)c3sc4ccccc4c3c2)c1 ; c1ccc(N(c2ccc(-c3ccc4ccccc4c3)cc2)c2cc(-c3ccccc3-n3c4ccccc4c4ccccc43)c3oc4ccccc4c3c2)c(-c2cccc3oc4ccccc4c23)c1]

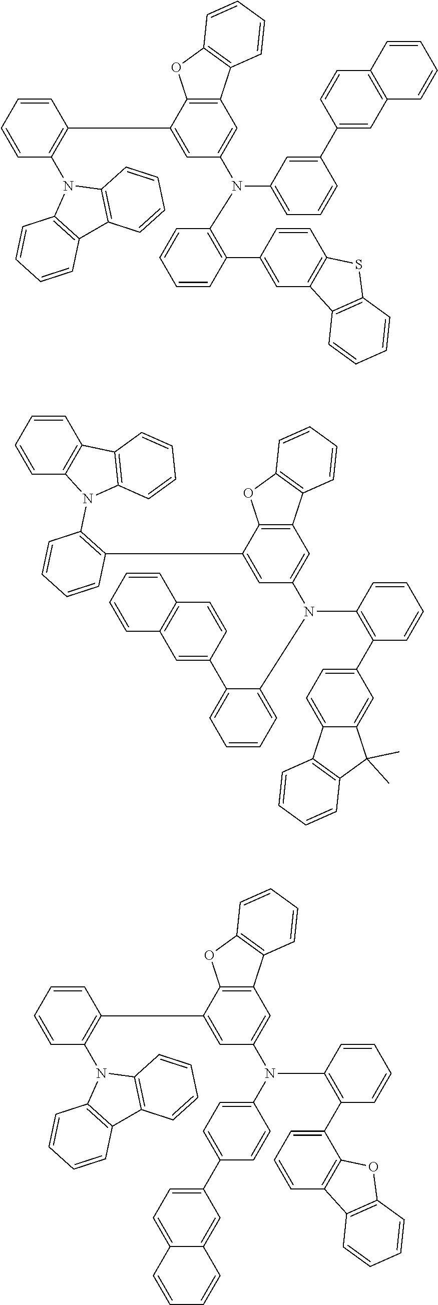 [SMILES: c1cc(-c2ccc3ccccc3c2)cc(N(c2cc(-c3ccccc3-n3c4ccccc4c4ccccc43)c3oc4ccccc4c3c2)c2ccccc2-c2ccc3sc4ccccc4c3c2)c1 ; CC1(C)c2ccccc2-c2ccc(-c3ccccc3N(c3cc(-c4ccccc4-n4c5ccccc5c5ccccc54)c4oc5ccccc5c4c3)c3ccccc3-c3ccc4ccccc4c3)cc21 ; c1ccc(N(c2ccc(-c3ccc4ccccc4c3)cc2)c2cc(-c3ccccc3-n3c4ccccc4c4ccccc43)c3oc4ccccc4c3c2)c(-c2cccc3c2oc2ccccc23)c1]

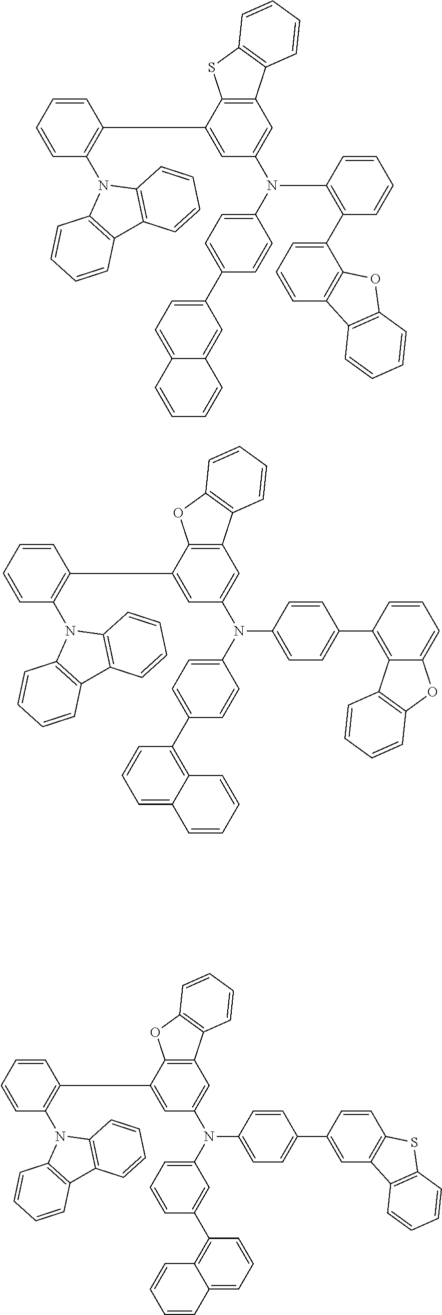

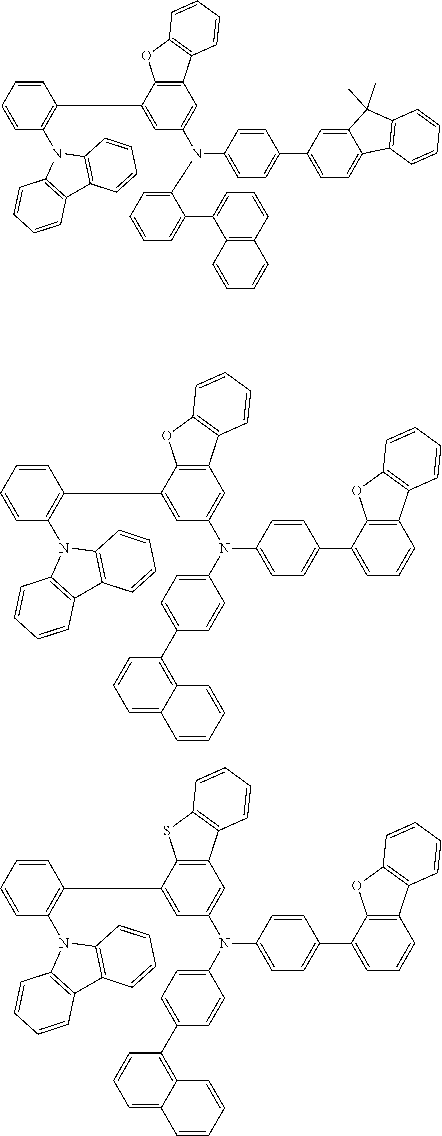

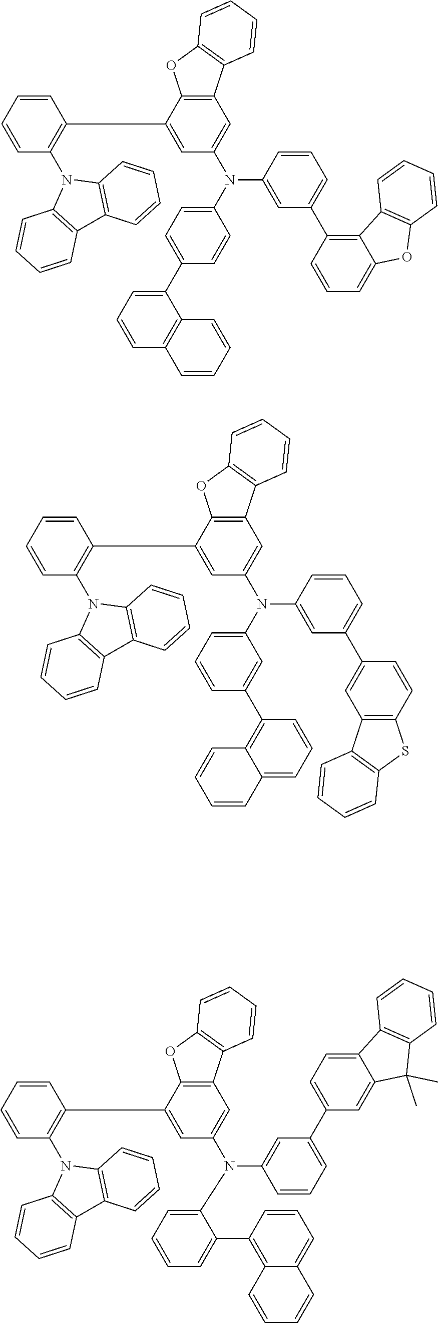 [SMILES: c1cc(-c2cccc3oc4ccccc4c23)cc(N(c2ccc(-c3cccc4ccccc34)cc2)c2cc(-c3ccccc3-n3c4ccccc4c4ccccc43)c3oc4ccccc4c3c2)c1 ; c1cc(-c2ccc3sc4ccccc4c3c2)cc(N(c2cccc(-c3cccc4ccccc34)c2)c2cc(-c3ccccc3-n3c4ccccc4c4ccccc43)c3oc4ccccc4c3c2)c1 ; CC1(C)c2ccccc2-c2ccc(-c3cccc(N(c4cc(-c5ccccc5-n5c6ccccc6c6ccccc65)c5oc6ccccc6c5c4)c4ccccc4-c4cccc5ccccc45)c3)cc21]

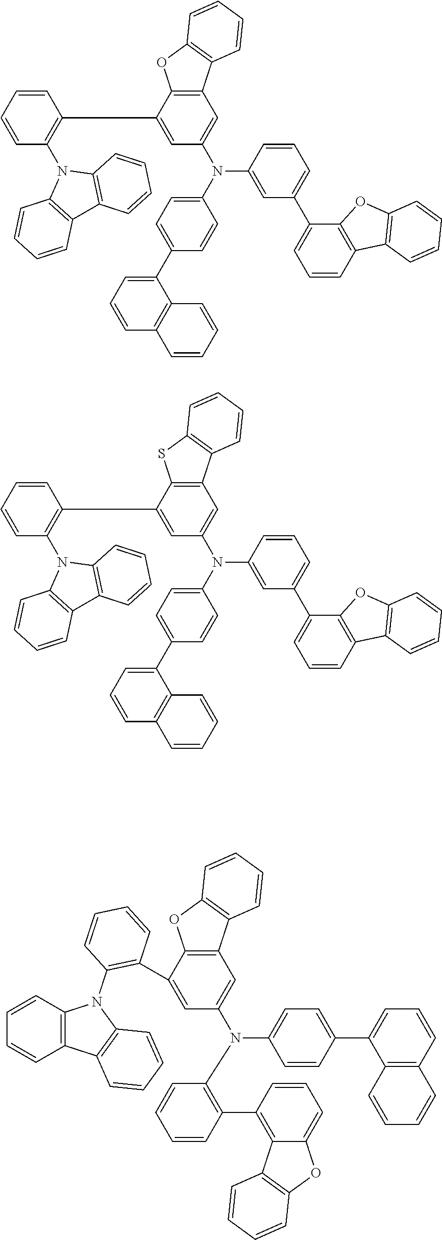

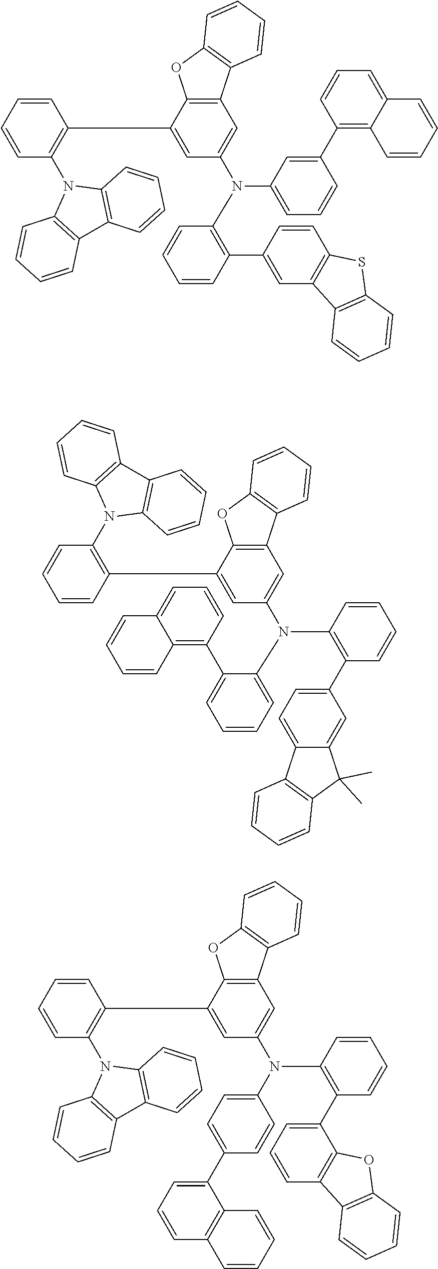

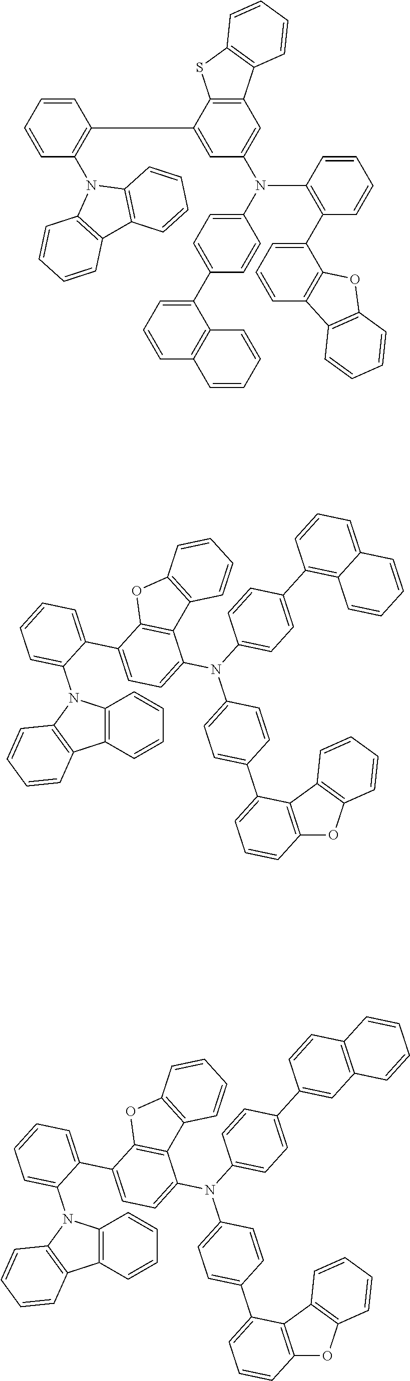 [SMILES: c1ccc(N(c2ccc(-c3cccc4ccccc34)cc2)c2cc(-c3ccccc3-n3c4ccccc4c4ccccc43)c3sc4ccccc4c3c2)c(-c2cccc3c2oc2ccccc23)c1 ; c1ccc(-n2c3ccccc3c3ccccc32)c(-c2ccc(N(c3ccc(-c4cccc5ccccc45)cc3)c3ccc(-c4cccc5oc6ccccc6c45)cc3)c3c2oc2ccccc23)c1 ; c1ccc(-n2c3ccccc3c3ccccc32)c(-c2ccc(N(c3ccc(-c4ccc5ccccc5c4)cc3)c3ccc(-c4cccc5oc6ccccc6c45)cc3)c3c2oc2ccccc23)c1]

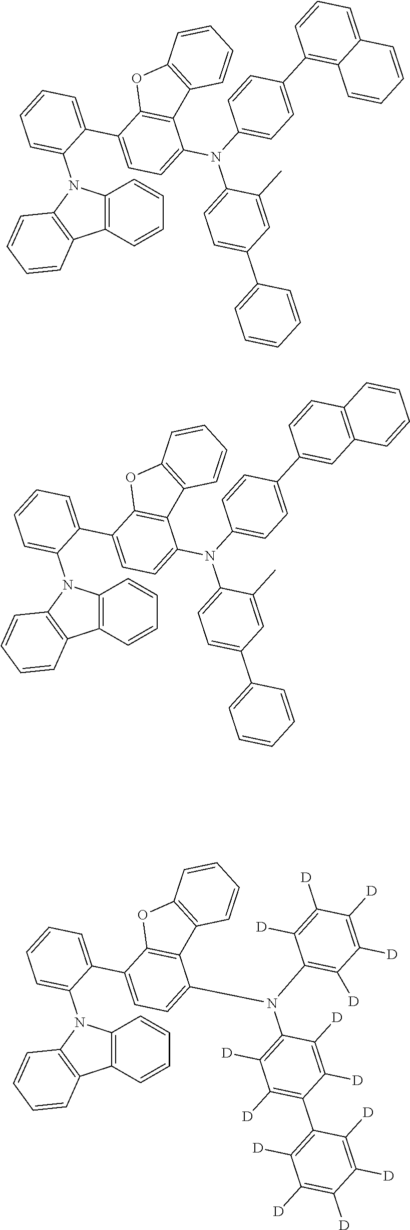 [SMILES: Cc1cc(-c2ccccc2)ccc1N(c1ccc(-c2cccc3ccccc23)cc1)c1ccc(-c2ccccc2-n2c3ccccc3c3ccccc32)c2oc3ccccc3c12 ; Cc1cc(-c2ccccc2)ccc1N(c1ccc(-c2ccc3ccccc3c2)cc1)c1ccc(-c2ccccc2-n2c3ccccc3c3ccccc32)c2oc3ccccc3c12 ; [2H]c1c([2H])c([2H])c(-c2c([2H])c([2H])c(N(c3c([2H])c([2H])c([2H])c([2H])c3[2H])c3ccc(-c4ccccc4-n4c5ccccc5c5ccccc54)c4oc5ccccc5c34)c([2H])c2[2H])c([2H])c1[2H]]

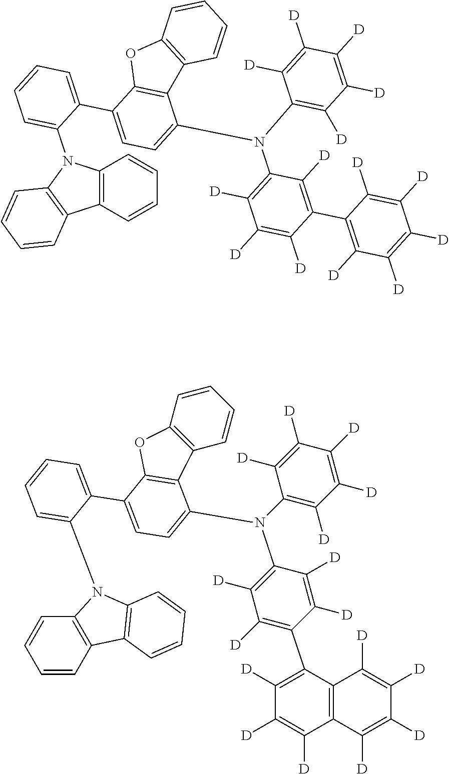 [SMILES: [2H]c1c([2H])c([2H])c(-c2c([2H])c([2H])c([2H])c(N(c3c([2H])c([2H])c([2H])c([2H])c3[2H])c3ccc(-c4ccccc4-n4c5ccccc5c5ccccc54)c4oc5ccccc5c34)c2[2H])c([2H])c1[2H] ; [2H]c1c([2H])c([2H])c(N(c2c([2H])c([2H])c(-c3c([2H])c([2H])c([2H])c4c([2H])c([2H])c([2H])c([2H])c34)c([2H])c2[2H])c2ccc(-c3ccccc3-n3c4ccccc4c4ccccc43)c3oc4ccccc4c23)c([2H])c1[2H]]

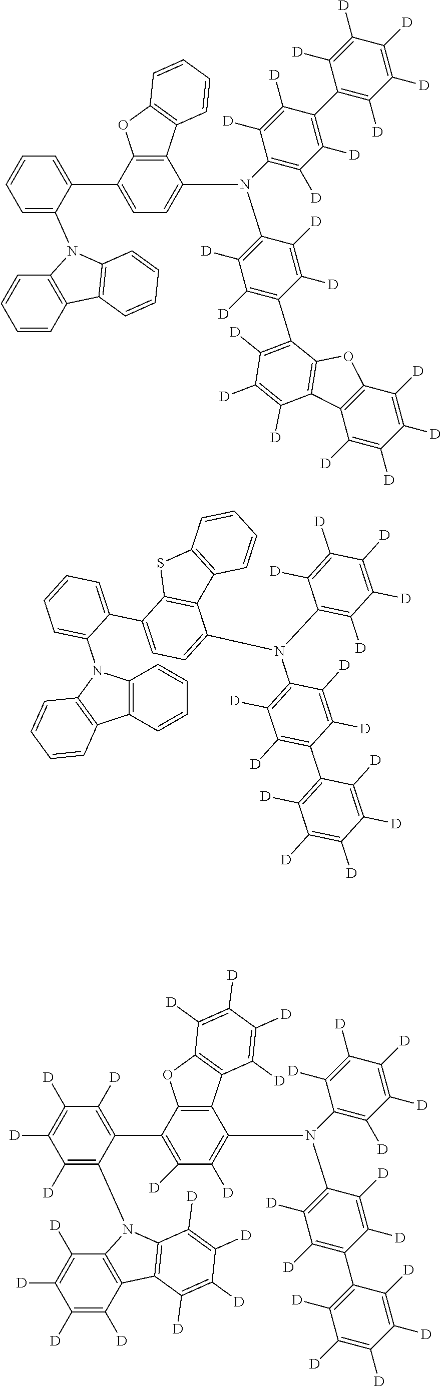

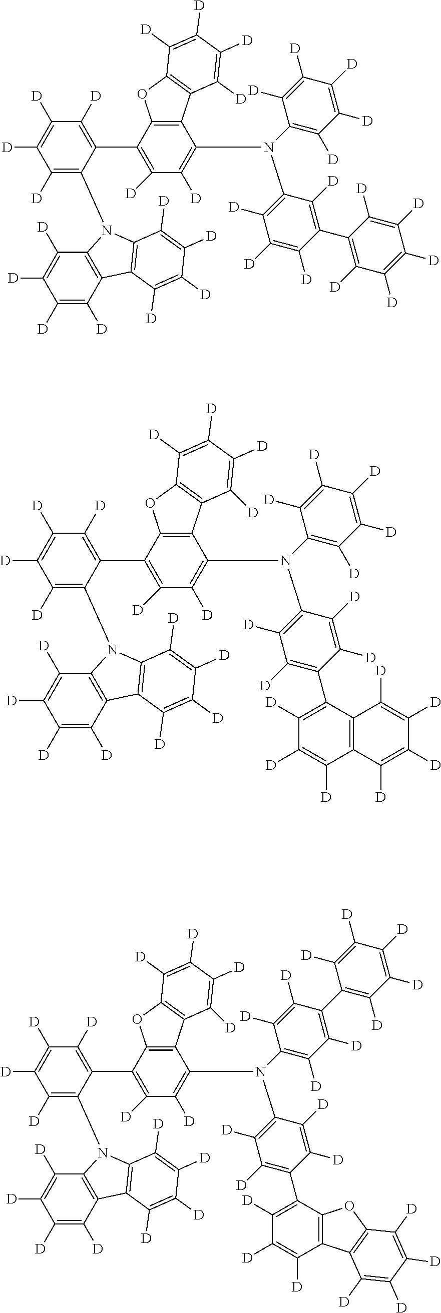

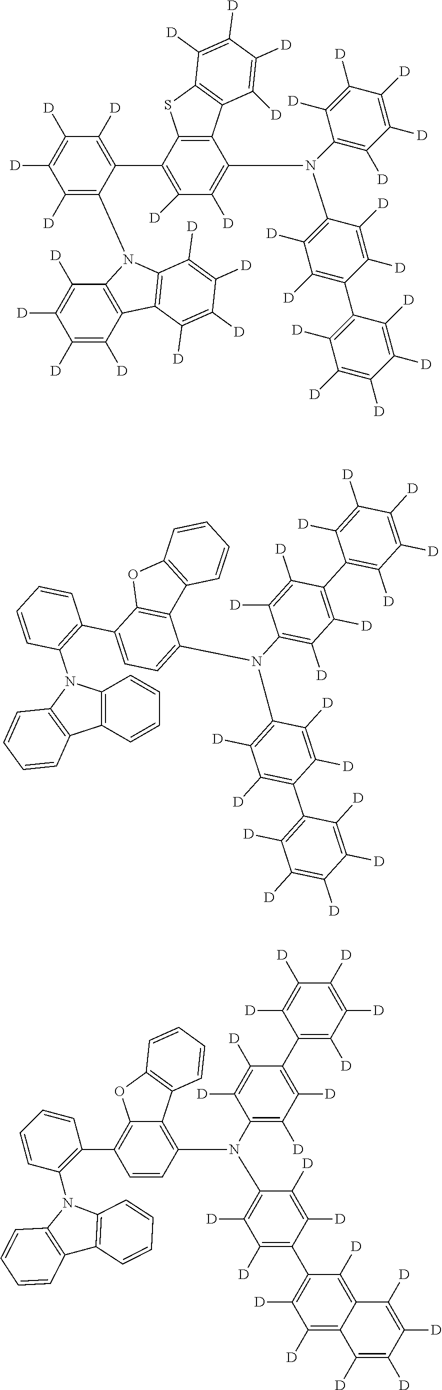

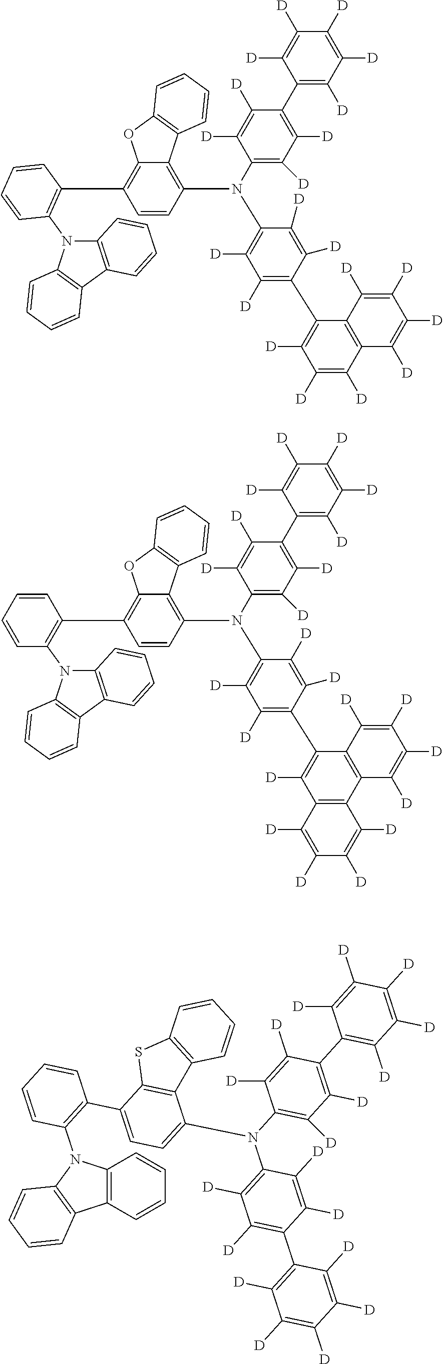

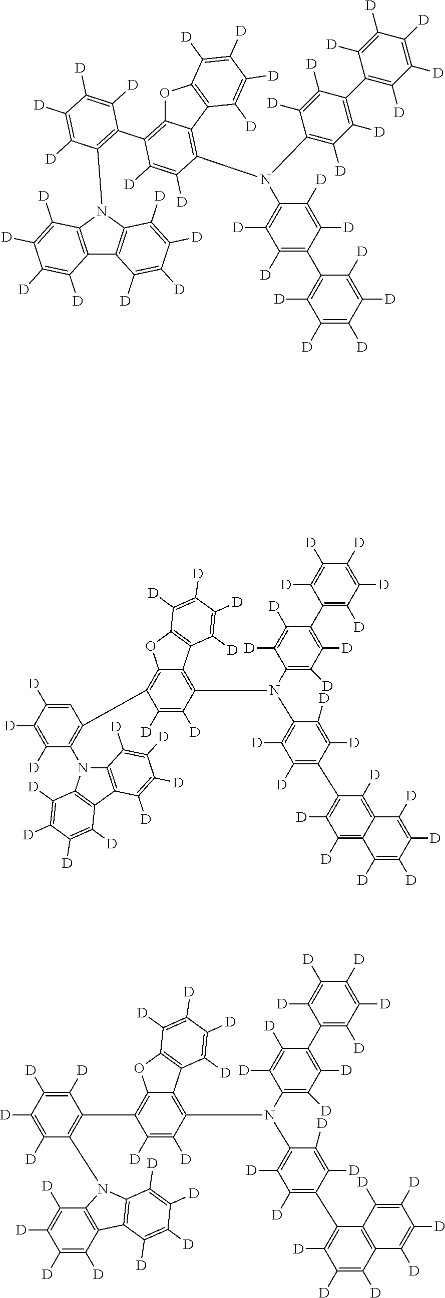

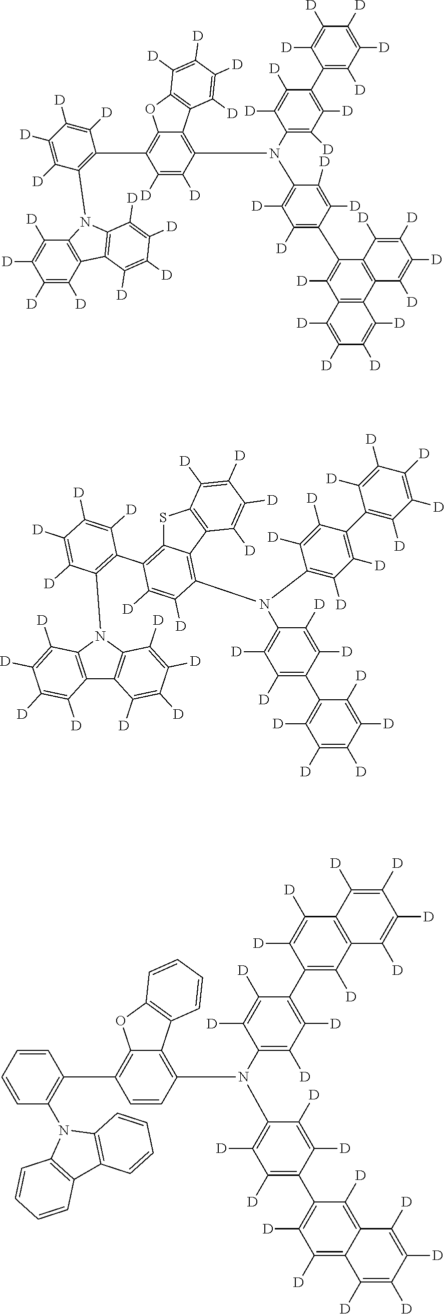

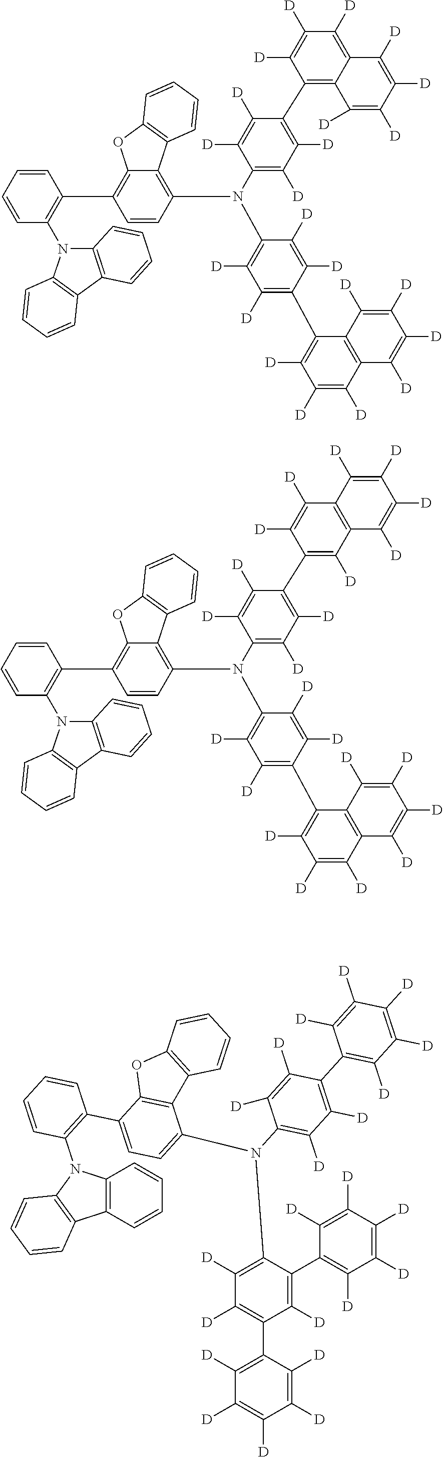

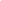

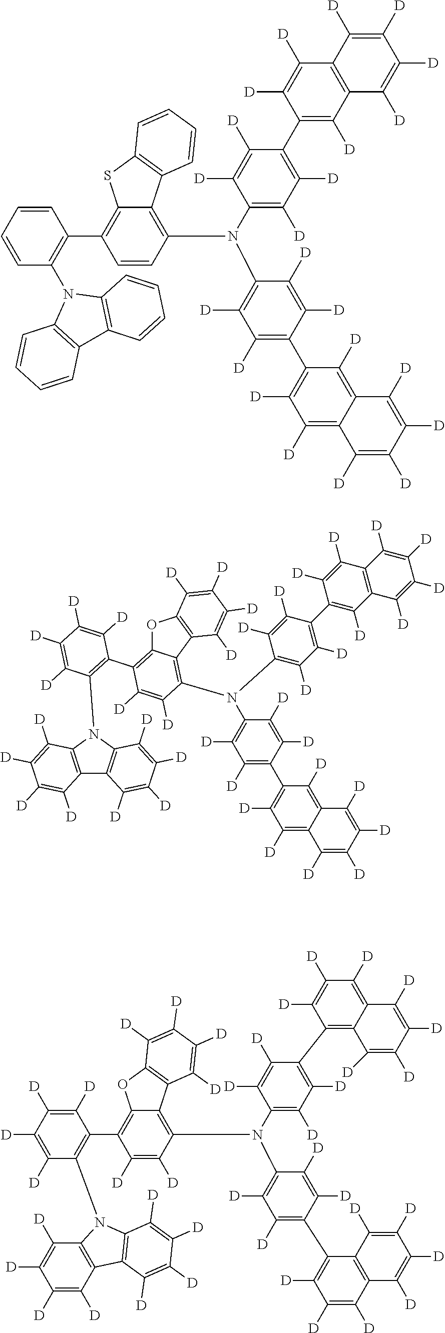

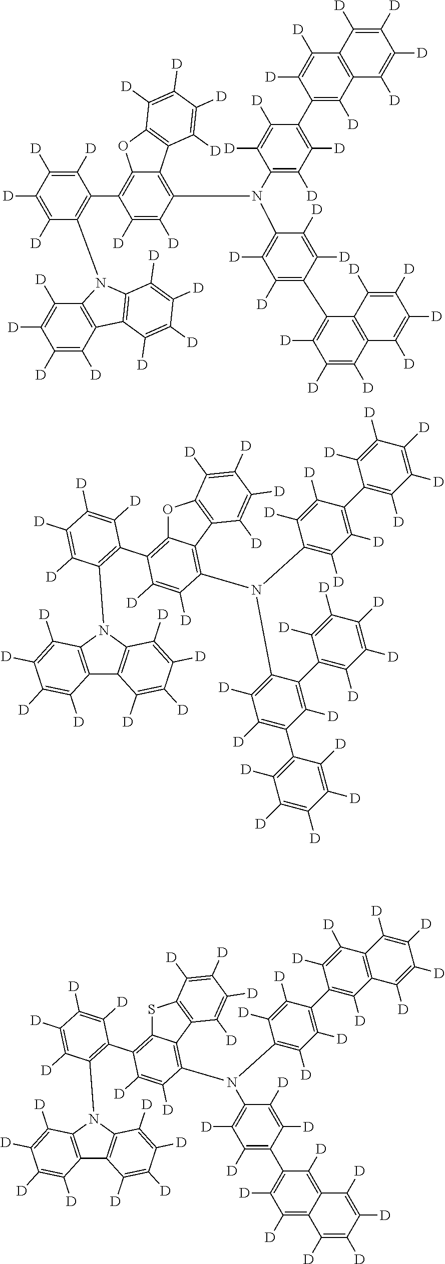

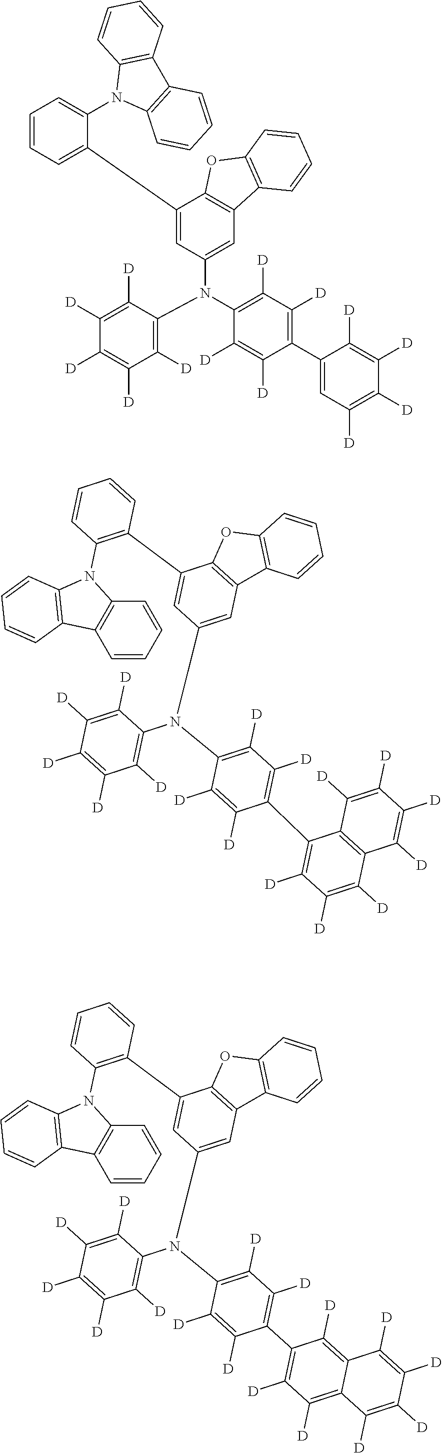

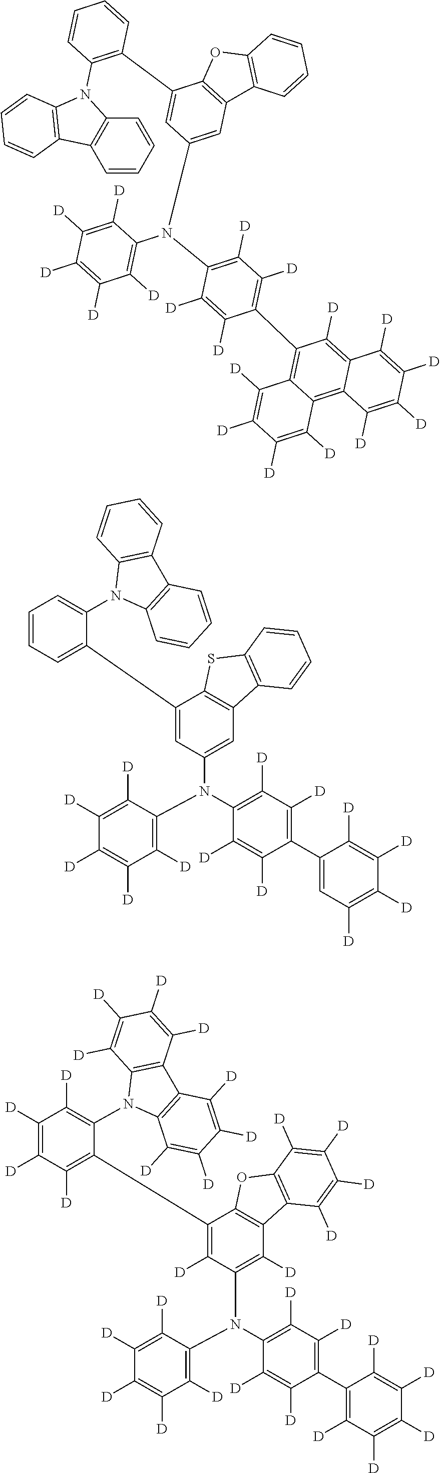

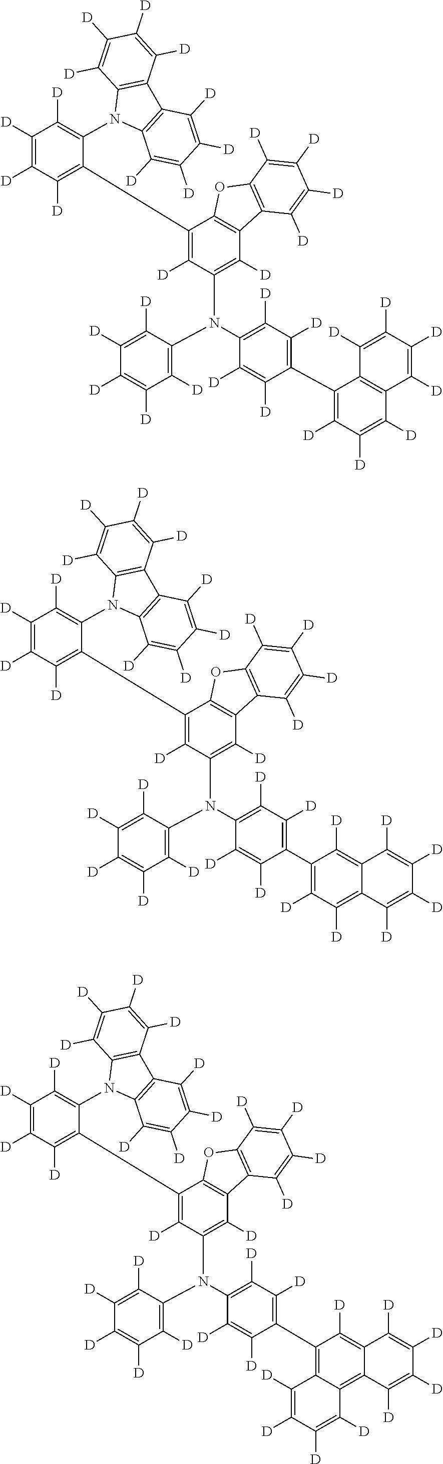

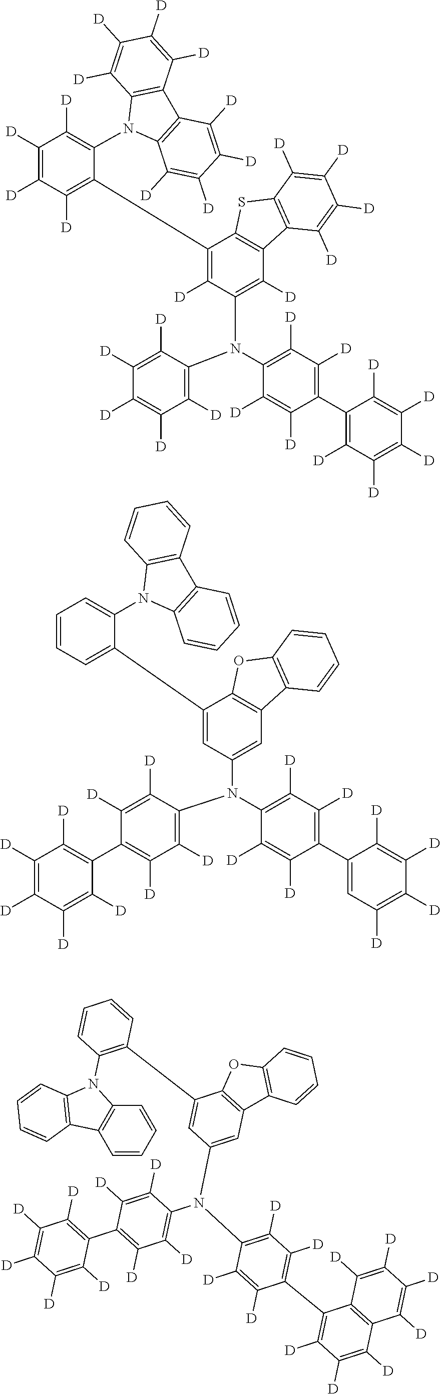

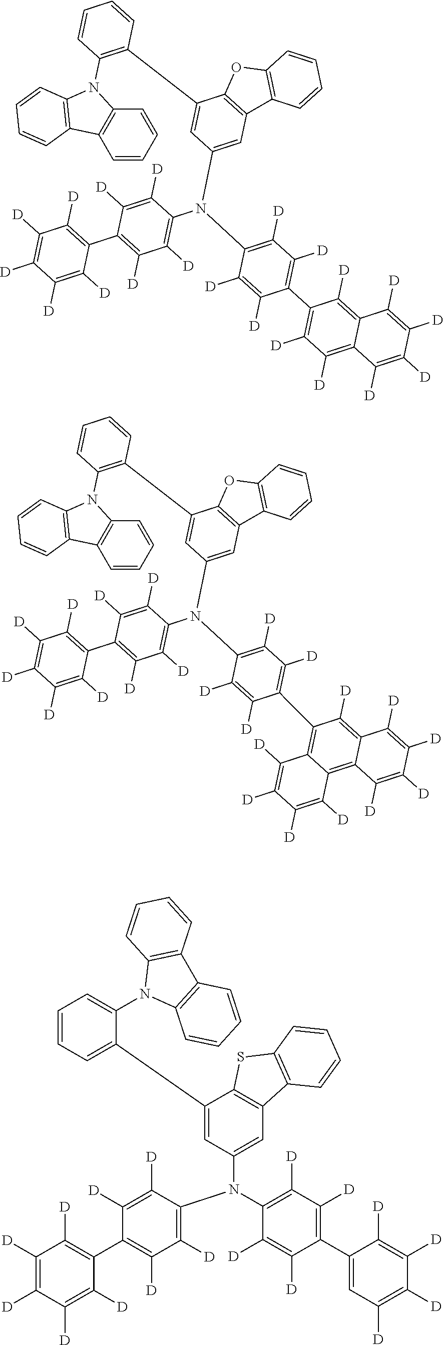

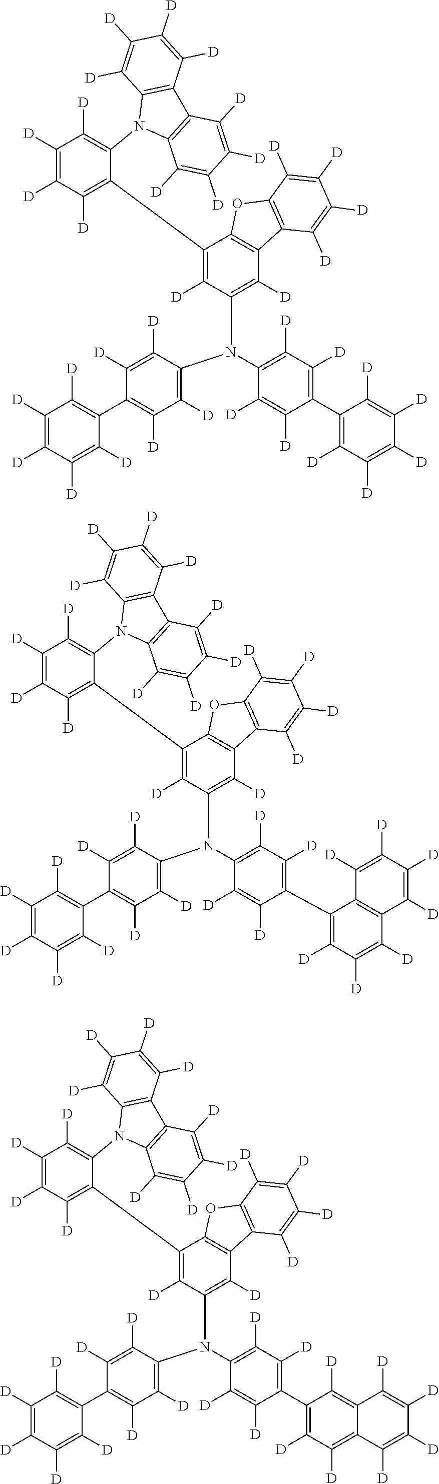

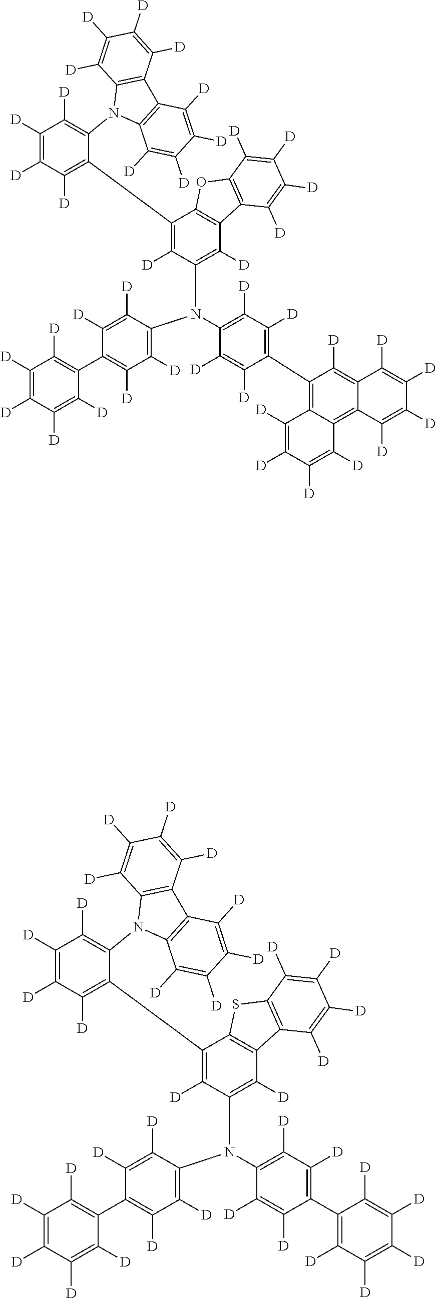

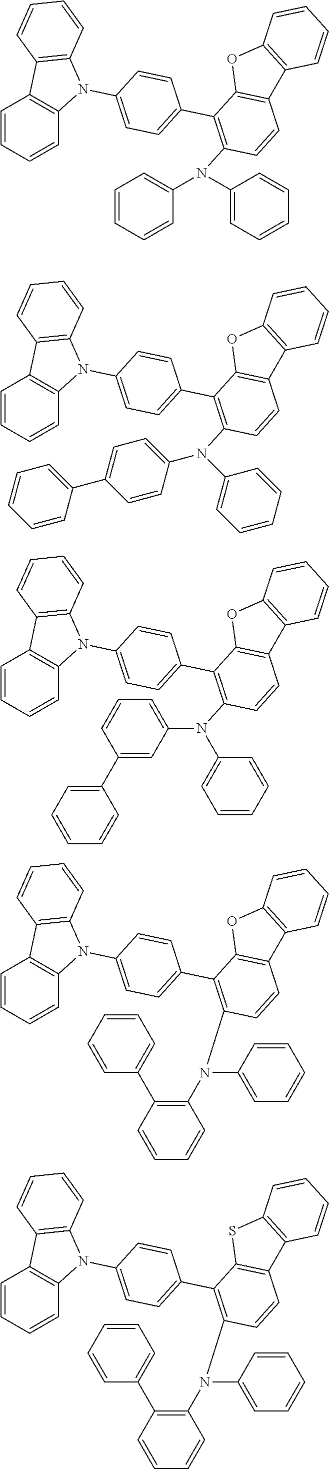

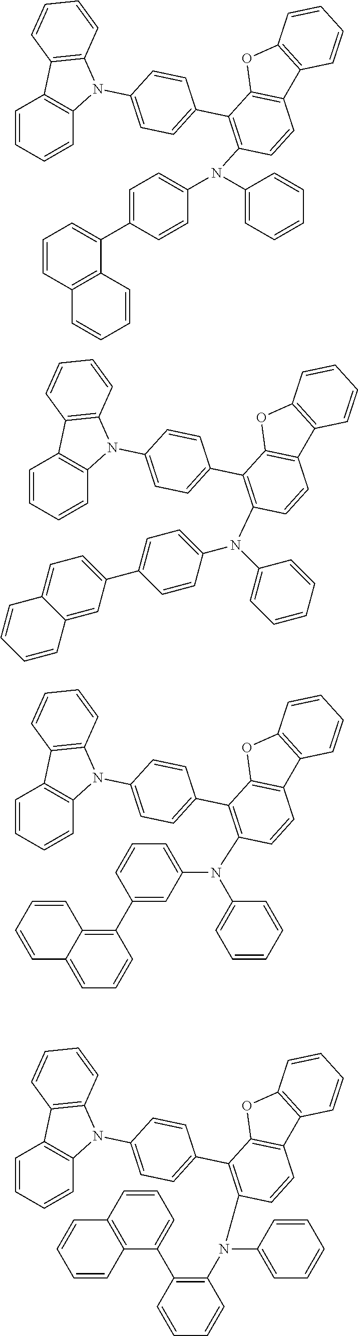

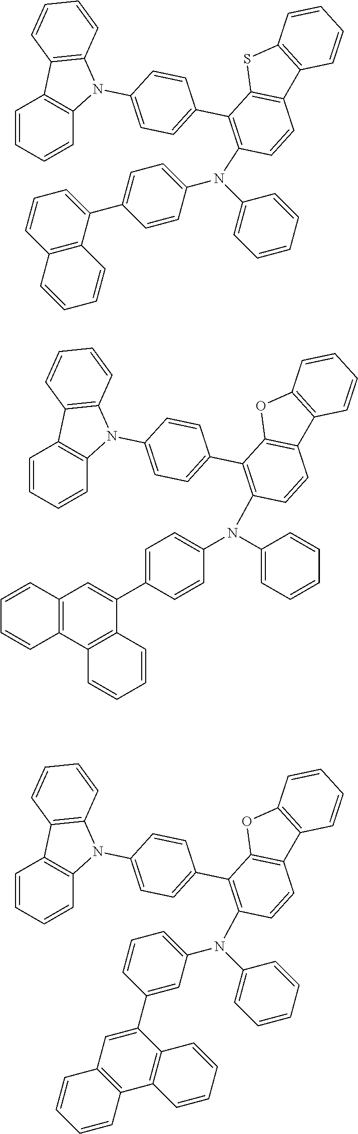

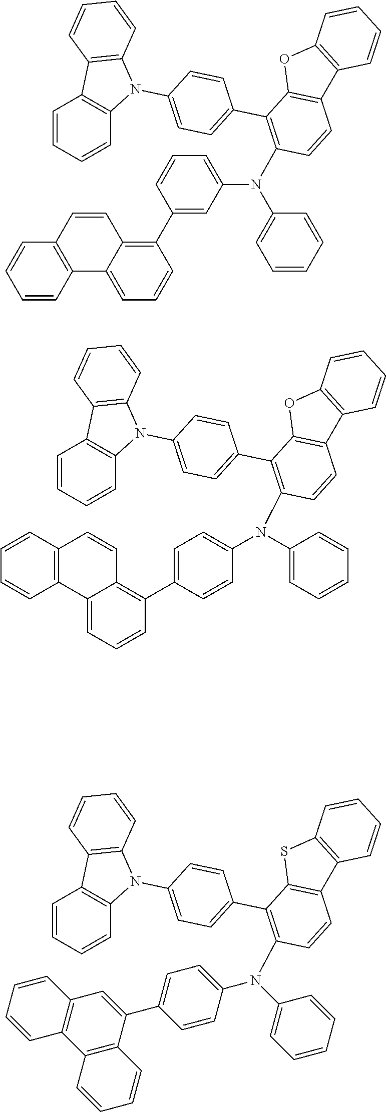

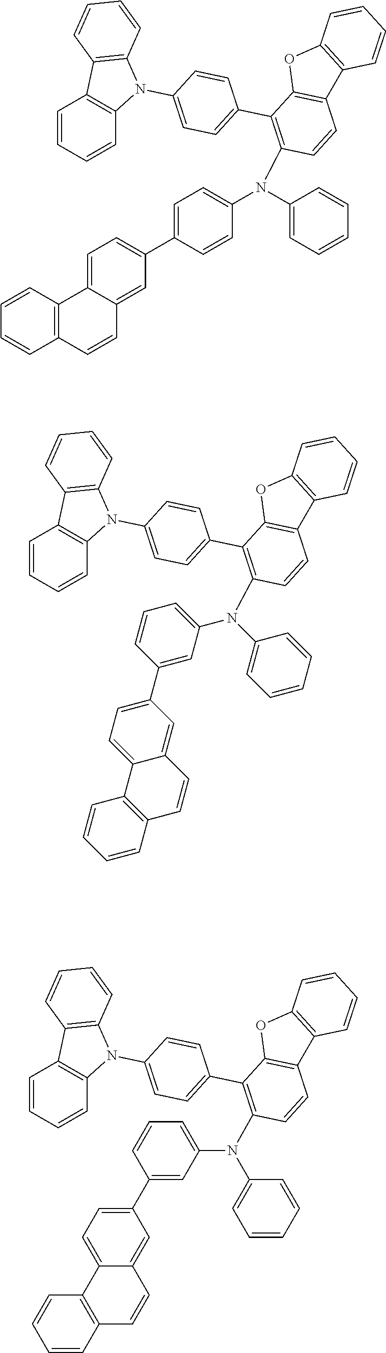 [SMILES: c1ccc(N(c2ccc(-c3ccc4c(ccc5ccccc54)c3)cc2)c2ccc3c(oc4ccccc43)c2-c2ccc(-n3c4ccccc4c4ccccc43)cc2)cc1 ; c1ccc(N(c2cccc(-c3ccc4c(ccc5ccccc54)c3)c2)c2ccc3c(oc4ccccc43)c2-c2ccc(-n3c4ccccc4c4ccccc43)cc2)cc1 ; c1ccc(N(c2cccc(-c3ccc4c(ccc5ccccc54)c3)c2)c2ccc3c(oc4ccccc43)c2-c2ccc(-n3c4ccccc4c4ccccc43)cc2)cc1]

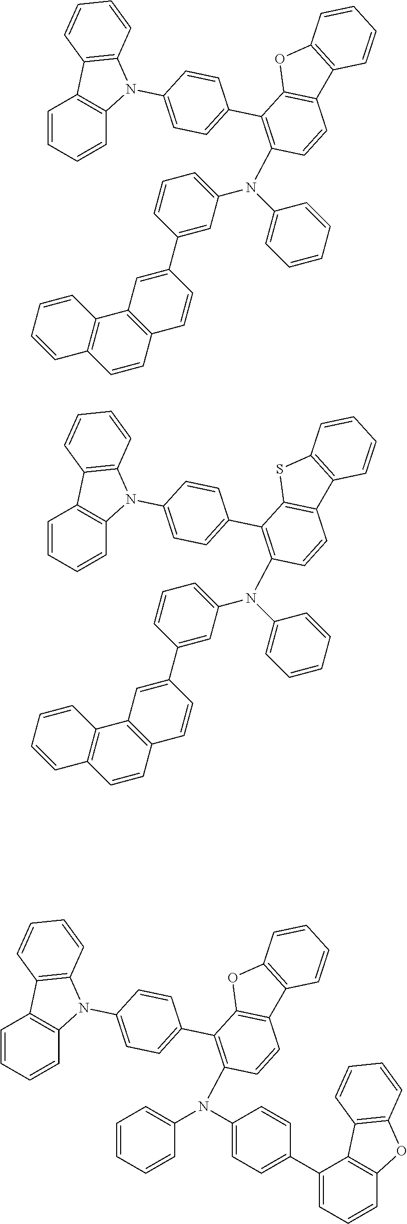

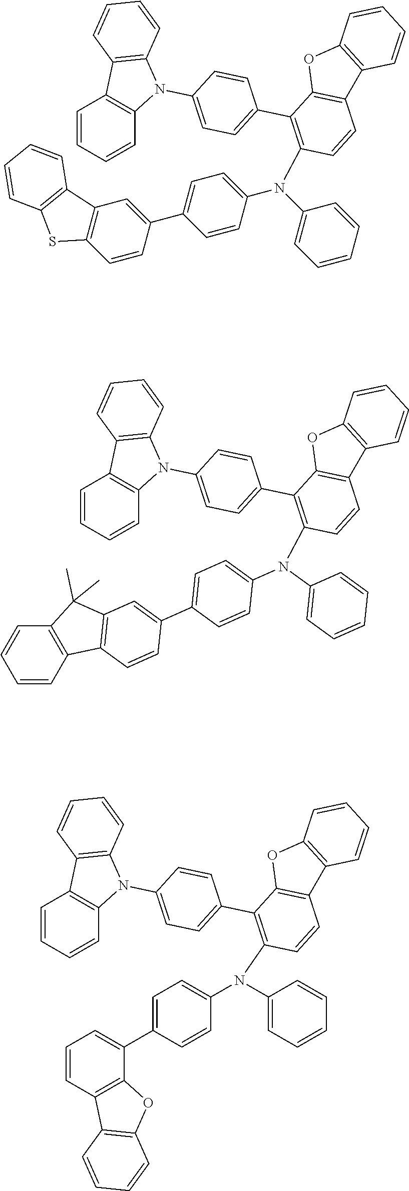 [SMILES: c1ccc(N(c2ccc(-c3ccc4sc5ccccc5c4c3)cc2)c2ccc3c(oc4ccccc43)c2-c2ccc(-n3c4ccccc4c4ccccc43)cc2)cc1 ; CC1(C)c2ccccc2-c2ccc(-c3ccc(N(c4ccccc4)c4ccc5c(oc6ccccc65)c4-c4ccc(-n5c6ccccc6c6ccccc65)cc4)cc3)cc21 ; c1ccc(N(c2ccc(-c3cccc4c3oc3ccccc34)cc2)c2ccc3c(oc4ccccc43)c2-c2ccc(-n3c4ccccc4c4ccccc43)cc2)cc1]

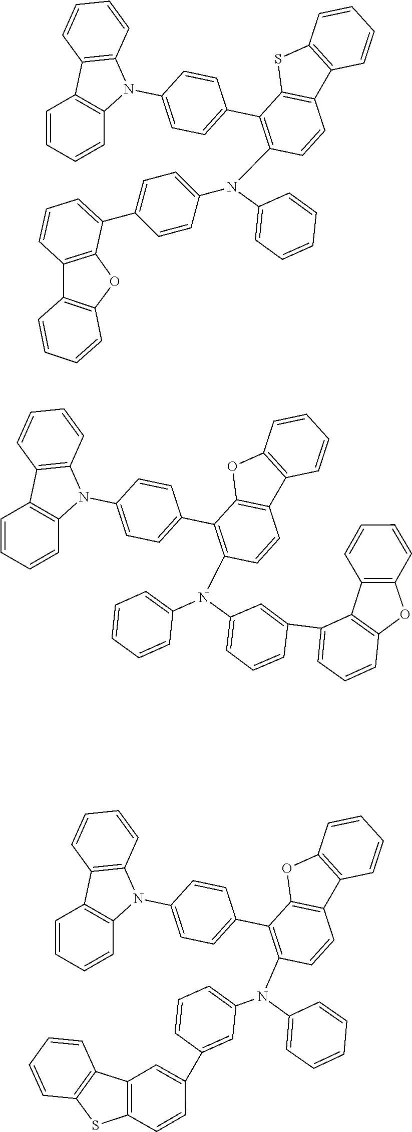 [SMILES: c1ccc(N(c2ccc(-c3cccc4c3oc3ccccc34)cc2)c2ccc3c(sc4ccccc43)c2-c2ccc(-n3c4ccccc4c4ccccc43)cc2)cc1 ; c1ccc(N(c2cccc(-c3cccc4oc5ccccc5c34)c2)c2ccc3c(oc4ccccc43)c2-c2ccc(-n3c4ccccc4c4ccccc43)cc2)cc1 ; c1ccc(N(c2cccc(-c3ccc4sc5ccccc5c4c3)c2)c2ccc3c(oc4ccccc43)c2-c2ccc(-n3c4ccccc4c4ccccc43)cc2)cc1]

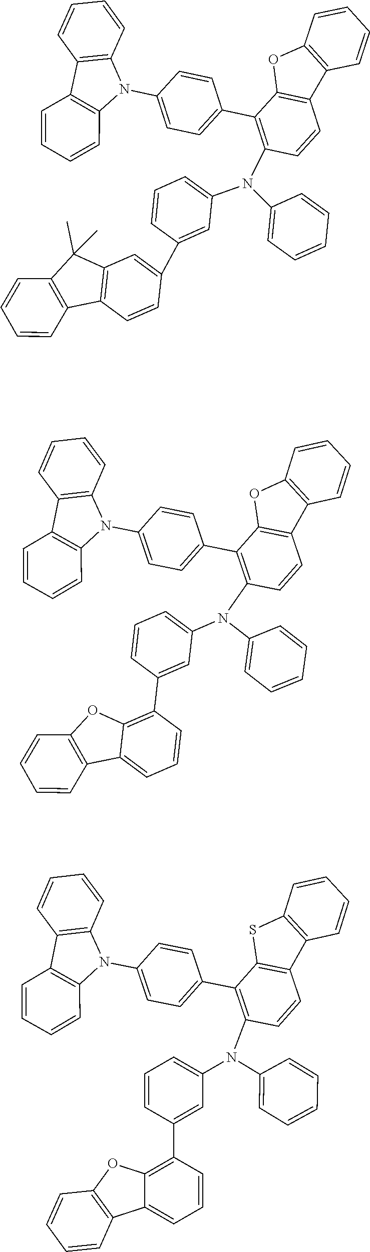 [SMILES: CC1(C)c2ccccc2-c2ccc(-c3cccc(N(c4ccccc4)c4ccc5c(oc6ccccc65)c4-c4ccc(-n5c6ccccc6c6ccccc65)cc4)c3)cc21 ; c1ccc(N(c2cccc(-c3cccc4c3oc3ccccc34)c2)c2ccc3c(oc4ccccc43)c2-c2ccc(-n3c4ccccc4c4ccccc43)cc2)cc1 ; c1ccc(N(c2cccc(-c3cccc4c3oc3ccccc34)c2)c2ccc3c(sc4ccccc43)c2-c2ccc(-n3c4ccccc4c4ccccc43)cc2)cc1]

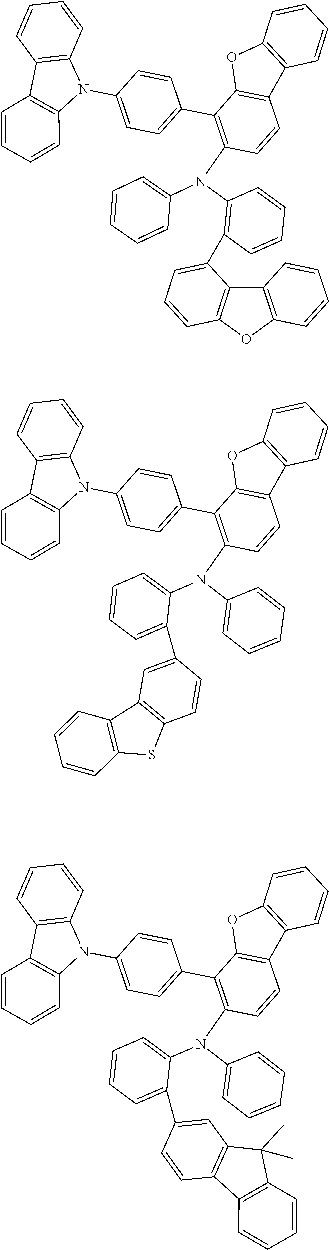 [SMILES: c1ccc(N(c2ccccc2-c2cccc3oc4ccccc4c23)c2ccc3c(oc4ccccc43)c2-c2ccc(-n3c4ccccc4c4ccccc43)cc2)cc1 ; c1ccc(N(c2ccccc2-c2ccc3sc4ccccc4c3c2)c2ccc3c(oc4ccccc43)c2-c2ccc(-n3c4ccccc4c4ccccc43)cc2)cc1 ; CC1(C)c2ccccc2-c2ccc(-c3ccccc3N(c3ccccc3)c3ccc4c(oc5ccccc54)c3-c3ccc(-n4c5ccccc5c5ccccc54)cc3)cc21]

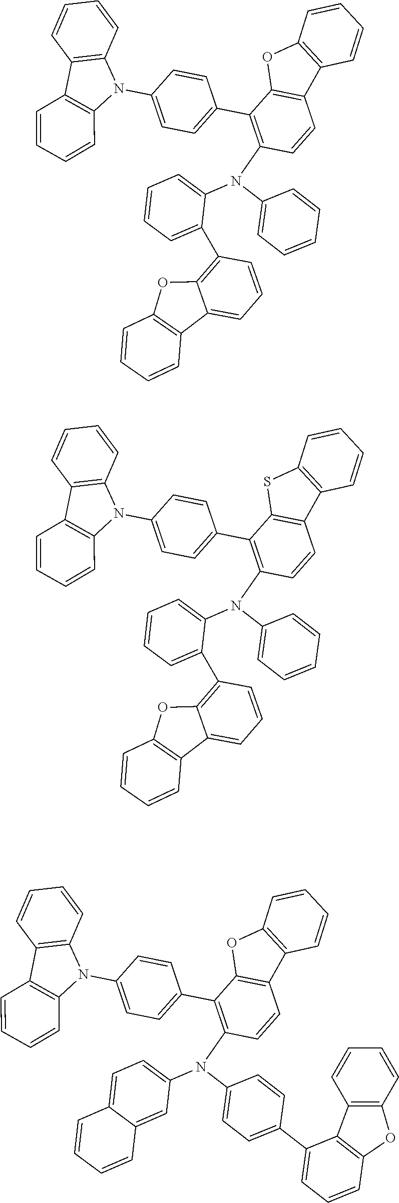 [SMILES: c1ccc(N(c2ccccc2-c2cccc3c2oc2ccccc23)c2ccc3c(oc4ccccc43)c2-c2ccc(-n3c4ccccc4c4ccccc43)cc2)cc1 ; c1ccc(N(c2ccccc2-c2cccc3c2oc2ccccc23)c2ccc3c(sc4ccccc43)c2-c2ccc(-n3c4ccccc4c4ccccc43)cc2)cc1 ; c1ccc2cc(N(c3ccc(-c4cccc5oc6ccccc6c45)cc3)c3ccc4c(oc5ccccc54)c3-c3ccc(-n4c5ccccc5c5ccccc54)cc3)ccc2c1]

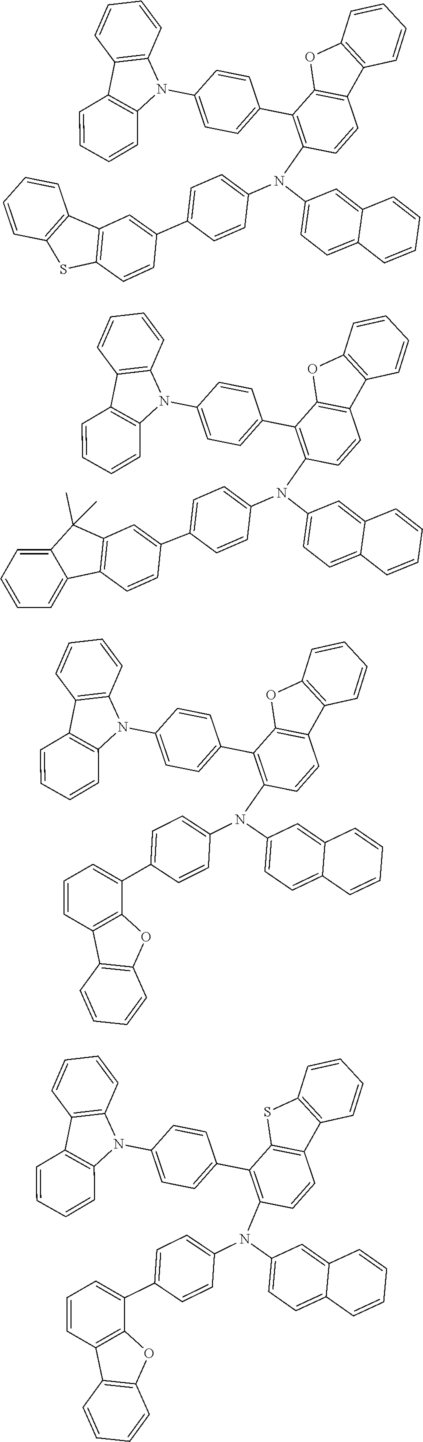

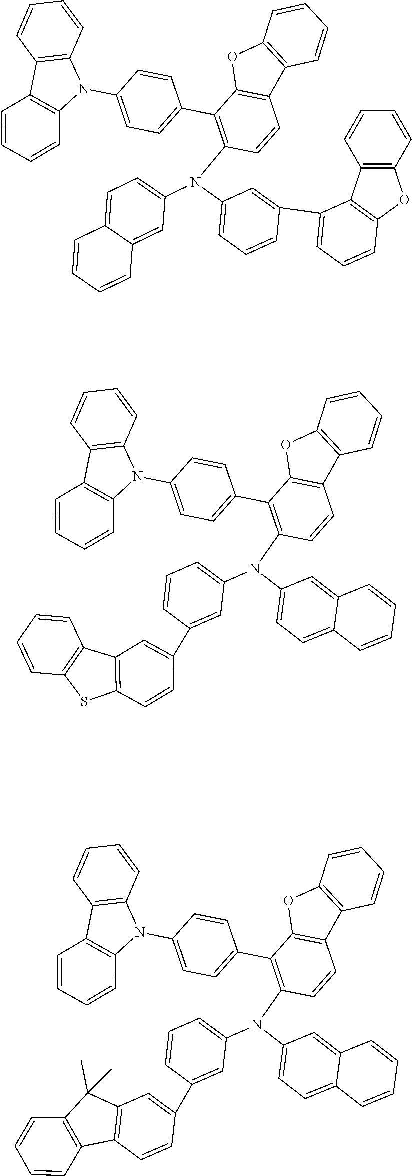 [SMILES: c1cc(-c2cccc3oc4ccccc4c23)cc(N(c2ccc3ccccc3c2)c2ccc3c(oc4ccccc43)c2-c2ccc(-n3c4ccccc4c4ccccc43)cc2)c1 ; c1cc(-c2ccc3sc4ccccc4c3c2)cc(N(c2ccc3ccccc3c2)c2ccc3c(oc4ccccc43)c2-c2ccc(-n3c4ccccc4c4ccccc43)cc2)c1 ; CC1(C)c2ccccc2-c2ccc(-c3cccc(N(c4ccc5ccccc5c4)c4ccc5c(oc6ccccc65)c4-c4ccc(-n5c6ccccc6c6ccccc65)cc4)c3)cc21]

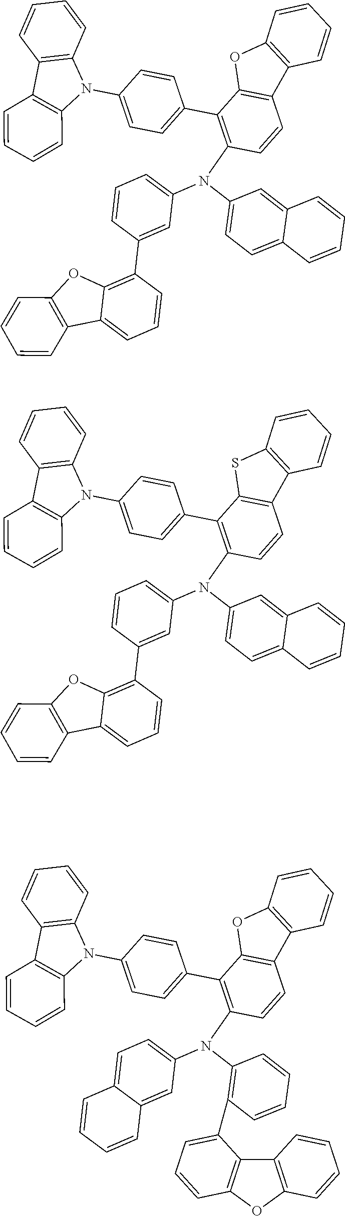 [SMILES: c1cc(-c2cccc3c2oc2ccccc23)cc(N(c2ccc3ccccc3c2)c2ccc3c(oc4ccccc43)c2-c2ccc(-n3c4ccccc4c4ccccc43)cc2)c1 ; c1cc(-c2cccc3c2oc2ccccc23)cc(N(c2ccc3ccccc3c2)c2ccc3c(sc4ccccc43)c2-c2ccc(-n3c4ccccc4c4ccccc43)cc2)c1 ; c1ccc(N(c2ccc3ccccc3c2)c2ccc3c(oc4ccccc43)c2-c2ccc(-n3c4ccccc4c4ccccc43)cc2)c(-c2cccc3oc4ccccc4c23)c1]

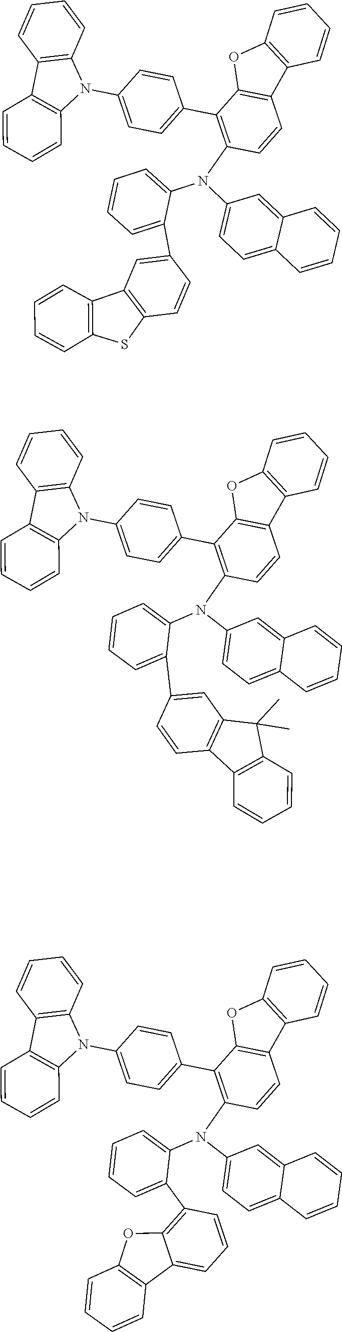 [SMILES: c1ccc(N(c2ccc3ccccc3c2)c2ccc3c(oc4ccccc43)c2-c2ccc(-n3c4ccccc4c4ccccc43)cc2)c(-c2ccc3sc4ccccc4c3c2)c1 ; CC1(C)c2ccccc2-c2ccc(-c3ccccc3N(c3ccc4ccccc4c3)c3ccc4c(oc5ccccc54)c3-c3ccc(-n4c5ccccc5c5ccccc54)cc3)cc21 ; c1ccc(N(c2ccc3ccccc3c2)c2ccc3c(oc4ccccc43)c2-c2ccc(-n3c4ccccc4c4ccccc43)cc2)c(-c2cccc3c2oc2ccccc23)c1]

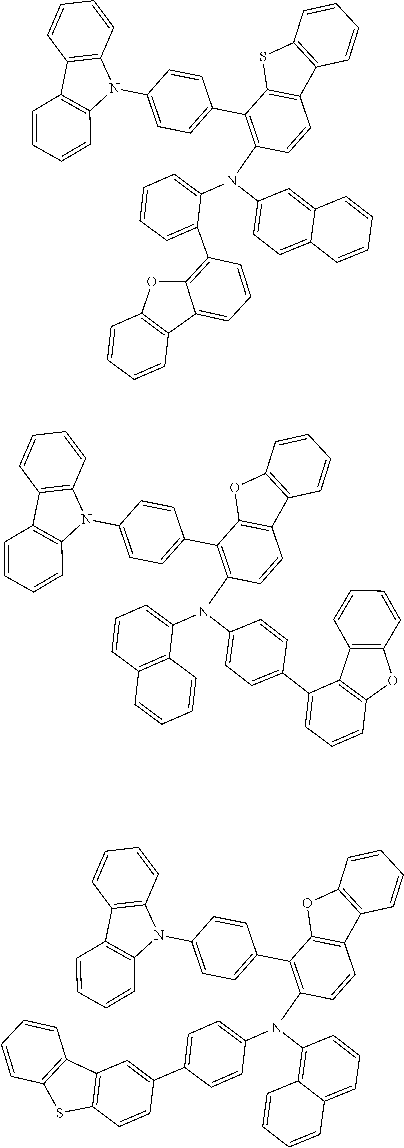 [SMILES: c1ccc(N(c2ccc3ccccc3c2)c2ccc3c(sc4ccccc43)c2-c2ccc(-n3c4ccccc4c4ccccc43)cc2)c(-c2cccc3c2oc2ccccc23)c1 ; c1ccc2c(N(c3ccc(-c4cccc5oc6ccccc6c45)cc3)c3ccc4c(oc5ccccc54)c3-c3ccc(-n4c5ccccc5c5ccccc54)cc3)cccc2c1 ; c1ccc2c(N(c3ccc(-c4ccc5sc6ccccc6c5c4)cc3)c3ccc4c(oc5ccccc54)c3-c3ccc(-n4c5ccccc5c5ccccc54)cc3)cccc2c1]

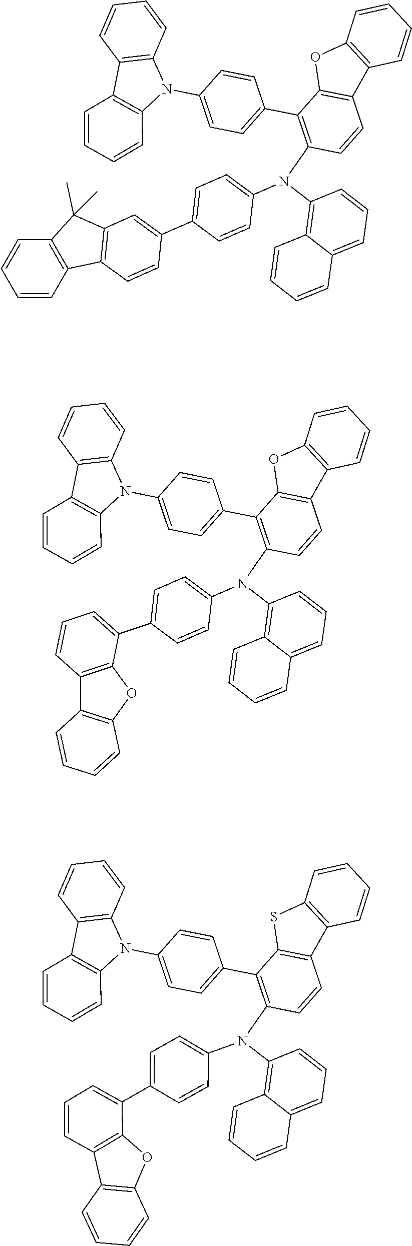 [SMILES: CC1(C)c2ccccc2-c2ccc(-c3ccc(N(c4ccc5c(oc6ccccc65)c4-c4ccc(-n5c6ccccc6c6ccccc65)cc4)c4cccc5ccccc45)cc3)cc21 ; c1ccc2c(N(c3ccc(-c4cccc5c4oc4ccccc45)cc3)c3ccc4c(oc5ccccc54)c3-c3ccc(-n4c5ccccc5c5ccccc54)cc3)cccc2c1 ; c1ccc2c(N(c3ccc(-c4cccc5c4oc4ccccc45)cc3)c3ccc4c(sc5ccccc54)c3-c3ccc(-n4c5ccccc5c5ccccc54)cc3)cccc2c1]

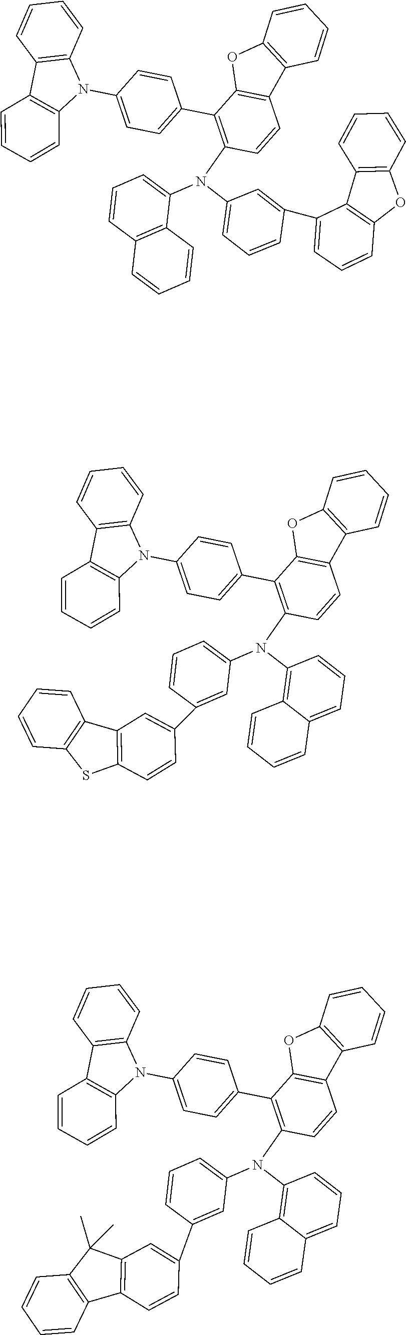 [SMILES: c1cc(-c2cccc3oc4ccccc4c23)cc(N(c2ccc3c(oc4ccccc43)c2-c2ccc(-n3c4ccccc4c4ccccc43)cc2)c2cccc3ccccc23)c1 ; c1cc(-c2ccc3sc4ccccc4c3c2)cc(N(c2ccc3c(oc4ccccc43)c2-c2ccc(-n3c4ccccc4c4ccccc43)cc2)c2cccc3ccccc23)c1 ; CC1(C)c2ccccc2-c2ccc(-c3cccc(N(c4ccc5c(oc6ccccc65)c4-c4ccc(-n5c6ccccc6c6ccccc65)cc4)c4cccc5ccccc45)c3)cc21]

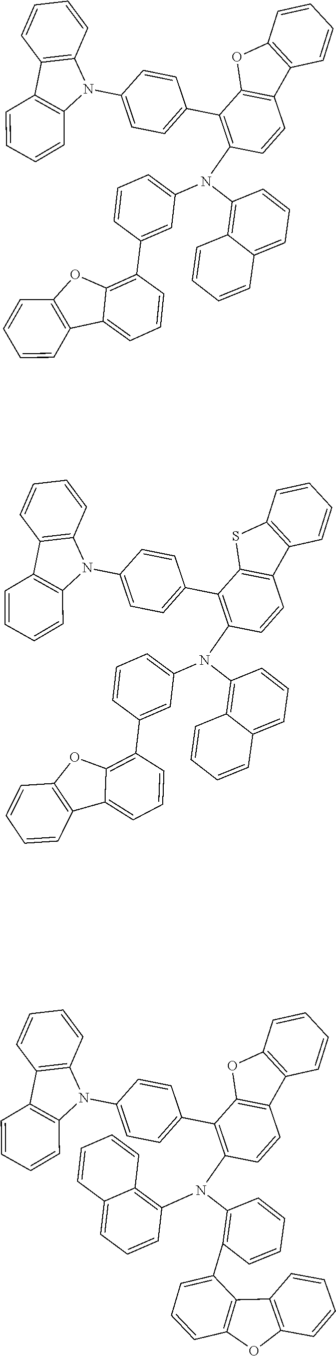 [SMILES: c1cc(-c2cccc3c2oc2ccccc23)cc(N(c2ccc3c(oc4ccccc43)c2-c2ccc(-n3c4ccccc4c4ccccc43)cc2)c2cccc3ccccc23)c1 ; c1cc(-c2cccc3c2oc2ccccc23)cc(N(c2ccc3c(sc4ccccc43)c2-c2ccc(-n3c4ccccc4c4ccccc43)cc2)c2cccc3ccccc23)c1 ; c1ccc(N(c2ccc3c(oc4ccccc43)c2-c2ccc(-n3c4ccccc4c4ccccc43)cc2)c2cccc3ccccc23)c(-c2cccc3oc4ccccc4c23)c1]

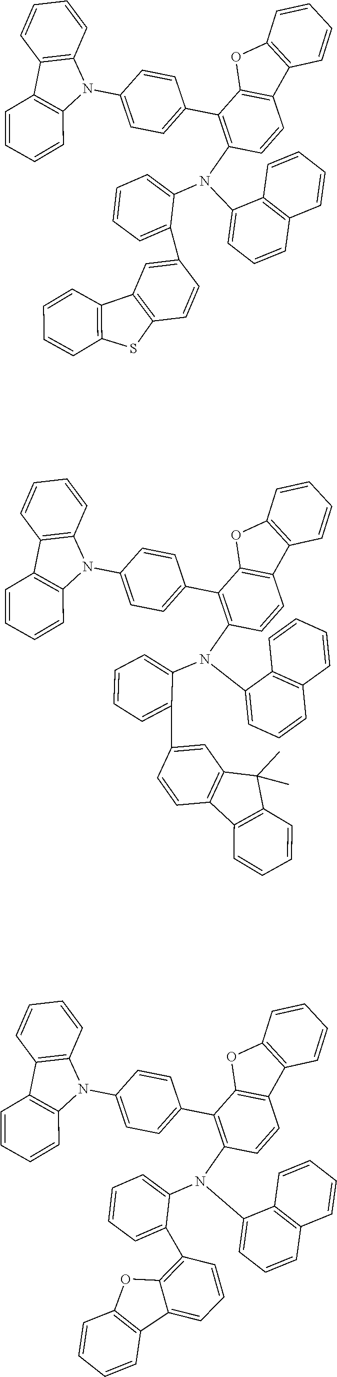 [SMILES: c1ccc(N(c2ccc3c(oc4ccccc43)c2-c2ccc(-n3c4ccccc4c4ccccc43)cc2)c2cccc3ccccc23)c(-c2ccc3sc4ccccc4c3c2)c1 ; CC1(C)c2ccccc2-c2ccc(-c3ccccc3N(c3ccc4c(oc5ccccc54)c3-c3ccc(-n4c5ccccc5c5ccccc54)cc3)c3cccc4ccccc34)cc21 ; c1ccc(N(c2ccc3c(oc4ccccc43)c2-c2ccc(-n3c4ccccc4c4ccccc43)cc2)c2cccc3ccccc23)c(-c2cccc3c2oc2ccccc23)c1]

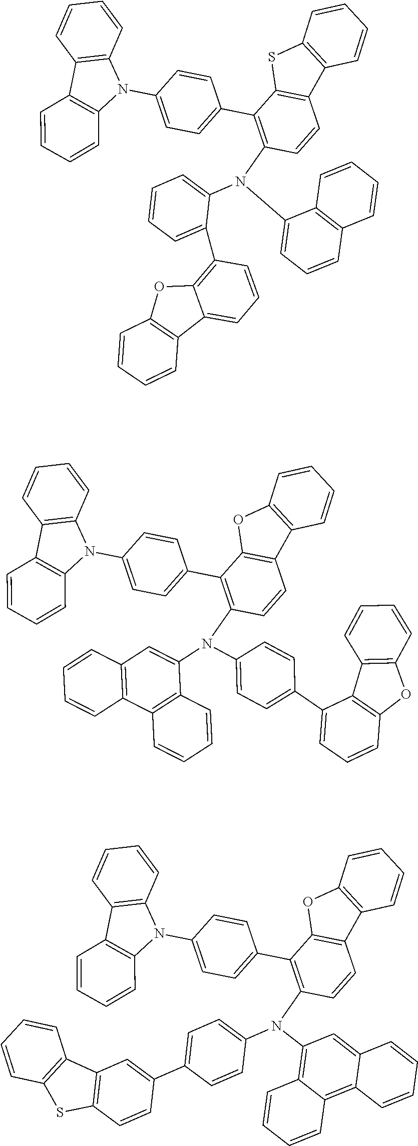 [SMILES: c1ccc(N(c2ccc3c(sc4ccccc43)c2-c2ccc(-n3c4ccccc4c4ccccc43)cc2)c2cccc3ccccc23)c(-c2cccc3c2oc2ccccc23)c1 ; c1ccc2c(c1)cc(N(c1ccc(-c3cccc4oc5ccccc5c34)cc1)c1ccc3c(oc4ccccc43)c1-c1ccc(-n3c4ccccc4c4ccccc43)cc1)c1ccccc12 ; c1ccc2c(c1)cc(N(c1ccc(-c3ccc4sc5ccccc5c4c3)cc1)c1ccc3c(oc4ccccc43)c1-c1ccc(-n3c4ccccc4c4ccccc43)cc1)c1ccccc12]

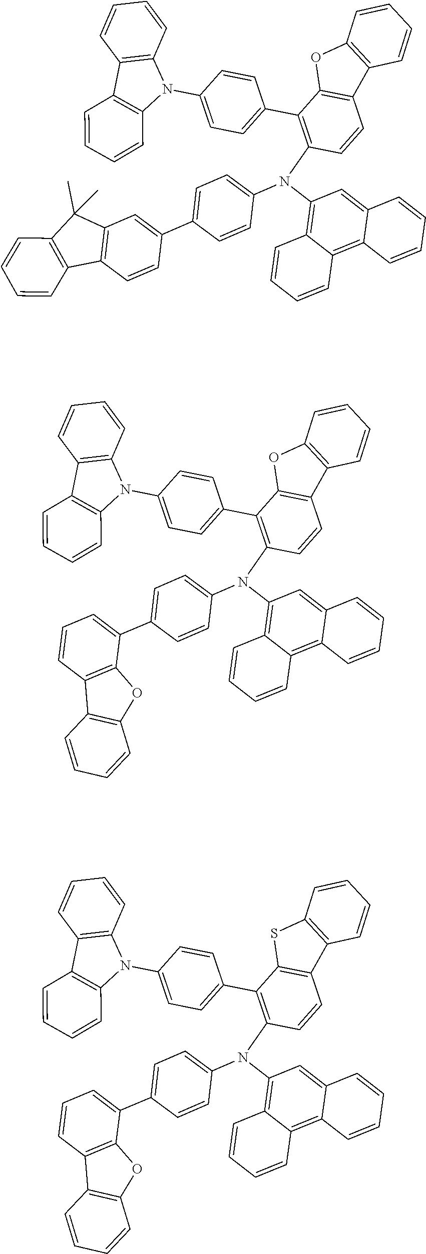 [SMILES: CC1(C)c2ccccc2-c2ccc(-c3ccc(N(c4ccc5c(oc6ccccc65)c4-c4ccc(-n5c6ccccc6c6ccccc65)cc4)c4cc5ccccc5c5ccccc45)cc3)cc21 ; c1ccc2c(c1)cc(N(c1ccc(-c3cccc4c3oc3ccccc34)cc1)c1ccc3c(oc4ccccc43)c1-c1ccc(-n3c4ccccc4c4ccccc43)cc1)c1ccccc12 ; c1ccc2c(c1)cc(N(c1ccc(-c3cccc4c3oc3ccccc34)cc1)c1ccc3c(sc4ccccc43)c1-c1ccc(-n3c4ccccc4c4ccccc43)cc1)c1ccccc12]

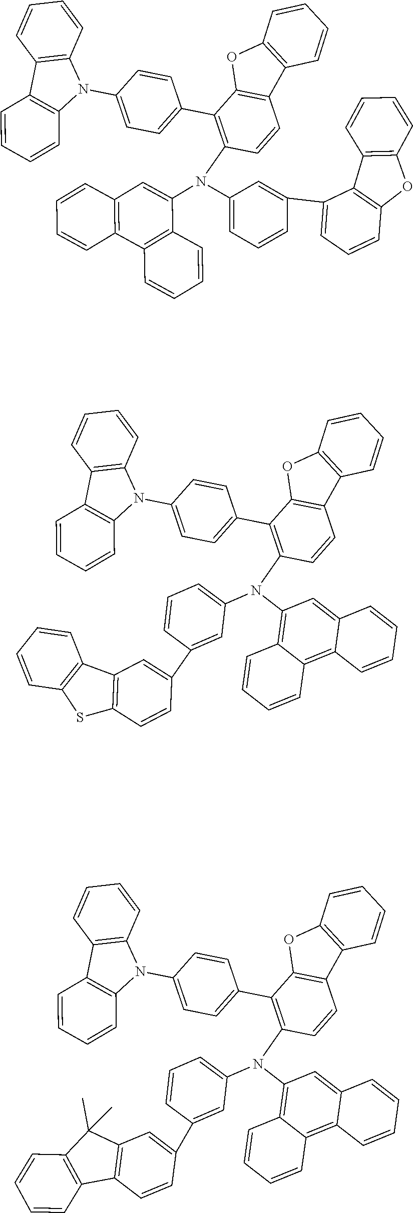 [SMILES: c1cc(-c2cccc3oc4ccccc4c23)cc(N(c2ccc3c(oc4ccccc43)c2-c2ccc(-n3c4ccccc4c4ccccc43)cc2)c2cc3ccccc3c3ccccc23)c1 ; c1cc(-c2ccc3sc4ccccc4c3c2)cc(N(c2ccc3c(oc4ccccc43)c2-c2ccc(-n3c4ccccc4c4ccccc43)cc2)c2cc3ccccc3c3ccccc23)c1 ; CC1(C)c2ccccc2-c2ccc(-c3cccc(N(c4ccc5c(oc6ccccc65)c4-c4ccc(-n5c6ccccc6c6ccccc65)cc4)c4cc5ccccc5c5ccccc45)c3)cc21]

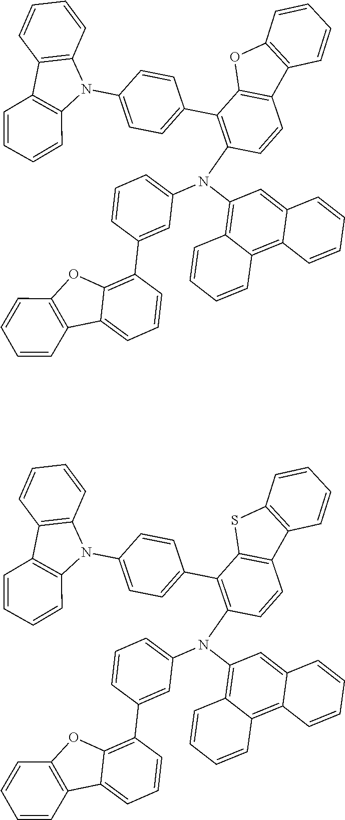 [SMILES: c1cc(-c2cccc3c2oc2ccccc23)cc(N(c2ccc3c(oc4ccccc43)c2-c2ccc(-n3c4ccccc4c4ccccc43)cc2)c2cc3ccccc3c3ccccc23)c1 ; c1cc(-c2cccc3c2oc2ccccc23)cc(N(c2ccc3c(sc4ccccc43)c2-c2ccc(-n3c4ccccc4c4ccccc43)cc2)c2cc3ccccc3c3ccccc23)c1]

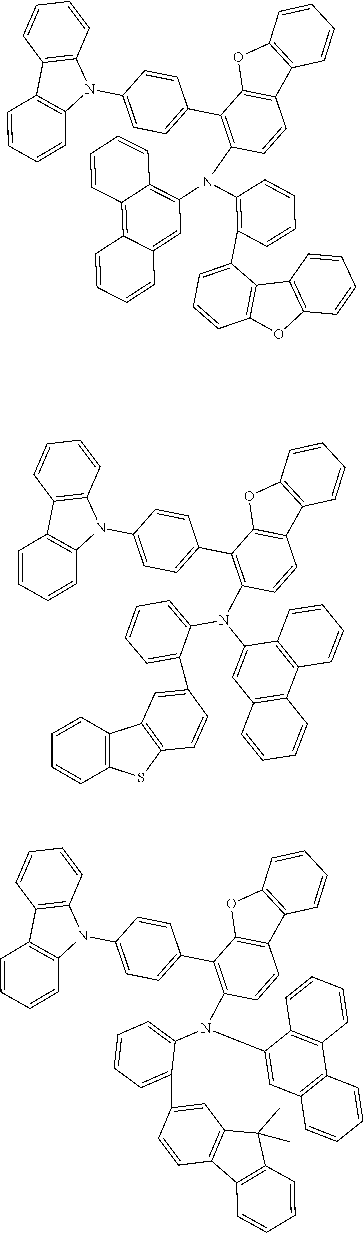 [SMILES: c1ccc(N(c2ccc3c(oc4ccccc43)c2-c2ccc(-n3c4ccccc4c4ccccc43)cc2)c2cc3ccccc3c3ccccc23)c(-c2cccc3oc4ccccc4c23)c1 ; c1ccc(N(c2ccc3c(oc4ccccc43)c2-c2ccc(-n3c4ccccc4c4ccccc43)cc2)c2cc3ccccc3c3ccccc23)c(-c2ccc3sc4ccccc4c3c2)c1 ; CC1(C)c2ccccc2-c2ccc(-c3ccccc3N(c3ccc4c(oc5ccccc54)c3-c3ccc(-n4c5ccccc5c5ccccc54)cc3)c3cc4ccccc4c4ccccc34)cc21]

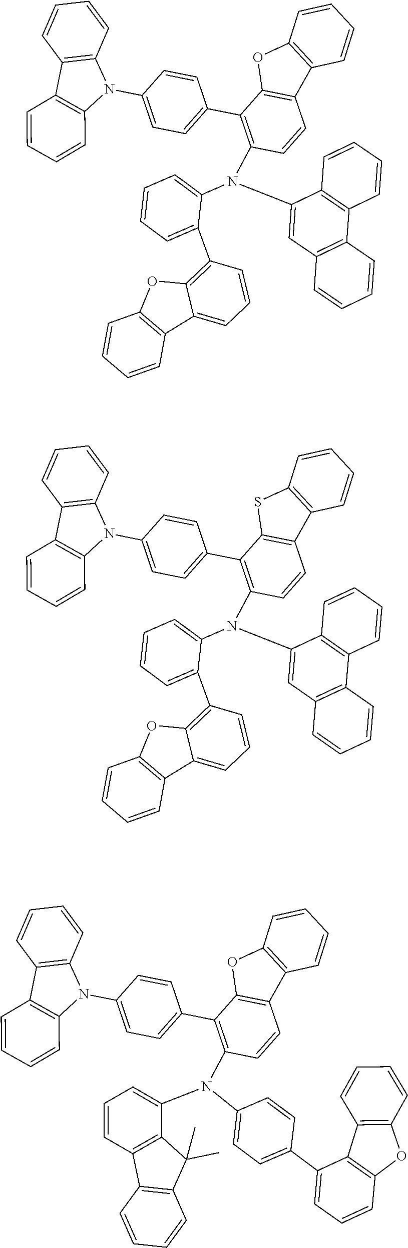 [SMILES: c1ccc(N(c2ccc3c(oc4ccccc43)c2-c2ccc(-n3c4ccccc4c4ccccc43)cc2)c2cc3ccccc3c3ccccc23)c(-c2cccc3c2oc2ccccc23)c1 ; c1ccc(N(c2ccc3c(sc4ccccc43)c2-c2ccc(-n3c4ccccc4c4ccccc43)cc2)c2cc3ccccc3c3ccccc23)c(-c2cccc3c2oc2ccccc23)c1 ; CC1(C)c2ccccc2-c2cccc(N(c3ccc(-c4cccc5oc6ccccc6c45)cc3)c3ccc4c(oc5ccccc54)c3-c3ccc(-n4c5ccccc5c5ccccc54)cc3)c21]

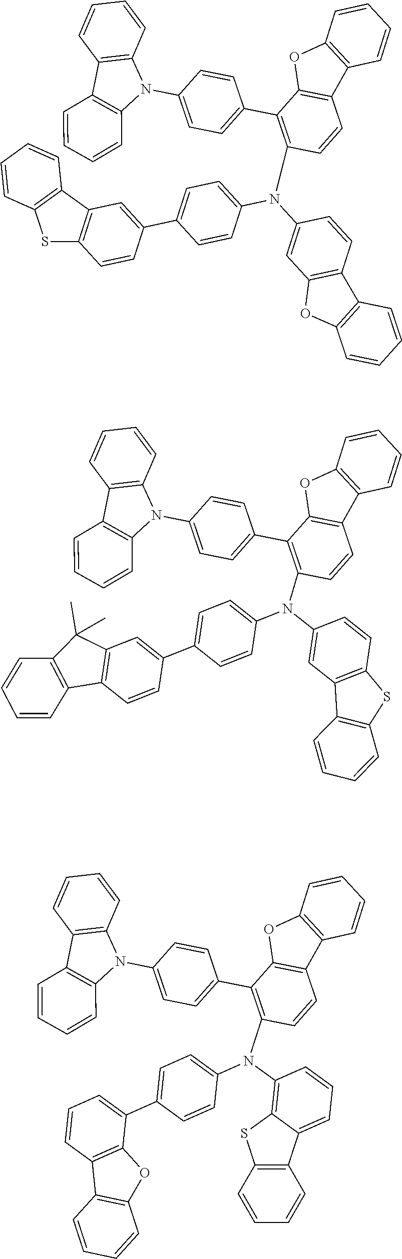 [SMILES: c1ccc2c(c1)oc1cc(N(c3ccc(-c4ccc5sc6ccccc6c5c4)cc3)c3ccc4c(oc5ccccc54)c3-c3ccc(-n4c5ccccc5c5ccccc54)cc3)ccc12 ; CC1(C)c2ccccc2-c2ccc(-c3ccc(N(c4ccc5sc6ccccc6c5c4)c4ccc5c(oc6ccccc65)c4-c4ccc(-n5c6ccccc6c6ccccc65)cc4)cc3)cc21 ; c1ccc2c(c1)oc1c(-c3ccc(N(c4ccc5c(oc6ccccc65)c4-c4ccc(-n5c6ccccc6c6ccccc65)cc4)c4cccc5c4sc4ccccc45)cc3)cccc12]

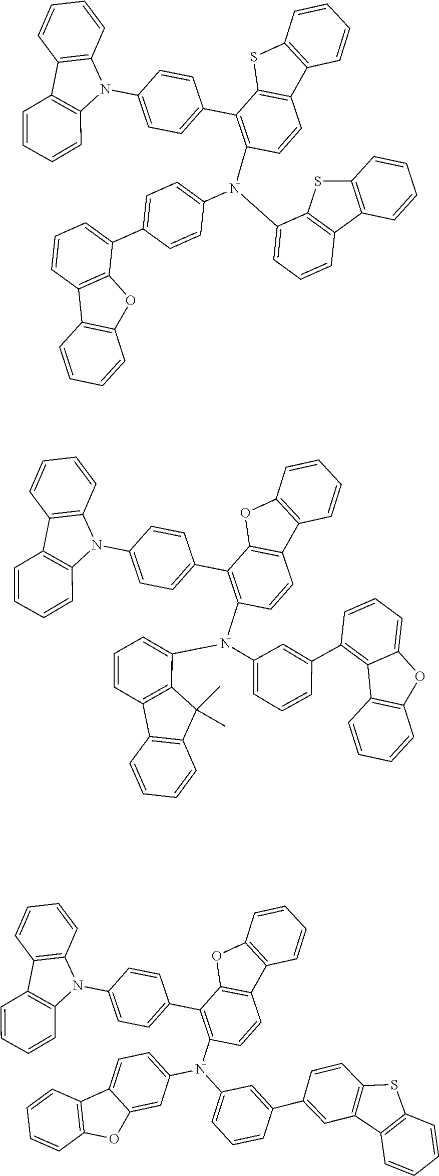 [SMILES: c1ccc2c(c1)oc1c(-c3ccc(N(c4ccc5c(sc6ccccc65)c4-c4ccc(-n5c6ccccc6c6ccccc65)cc4)c4cccc5c4sc4ccccc45)cc3)cccc12 ; CC1(C)c2ccccc2-c2cccc(N(c3cccc(-c4cccc5oc6ccccc6c45)c3)c3ccc4c(oc5ccccc54)c3-c3ccc(-n4c5ccccc5c5ccccc54)cc3)c21 ; c1cc(-c2ccc3sc4ccccc4c3c2)cc(N(c2ccc3c(c2)oc2ccccc23)c2ccc3c(oc4ccccc43)c2-c2ccc(-n3c4ccccc4c4ccccc43)cc2)c1]

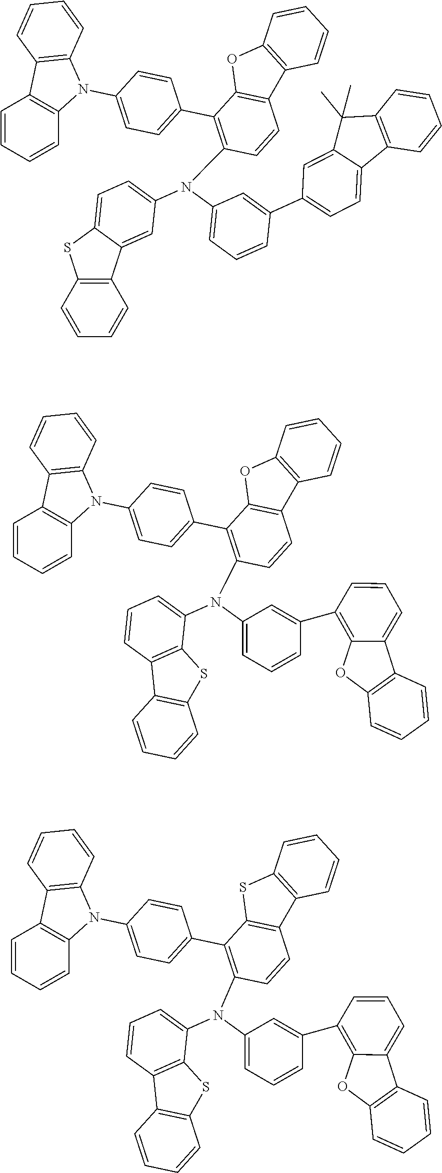 [SMILES: CC1(C)c2ccccc2-c2ccc(-c3cccc(N(c4ccc5sc6ccccc6c5c4)c4ccc5c(oc6ccccc65)c4-c4ccc(-n5c6ccccc6c6ccccc65)cc4)c3)cc21 ; c1cc(-c2cccc3c2oc2ccccc23)cc(N(c2ccc3c(oc4ccccc43)c2-c2ccc(-n3c4ccccc4c4ccccc43)cc2)c2cccc3c2sc2ccccc23)c1 ; c1cc(-c2cccc3c2oc2ccccc23)cc(N(c2ccc3c(sc4ccccc43)c2-c2ccc(-n3c4ccccc4c4ccccc43)cc2)c2cccc3c2sc2ccccc23)c1]

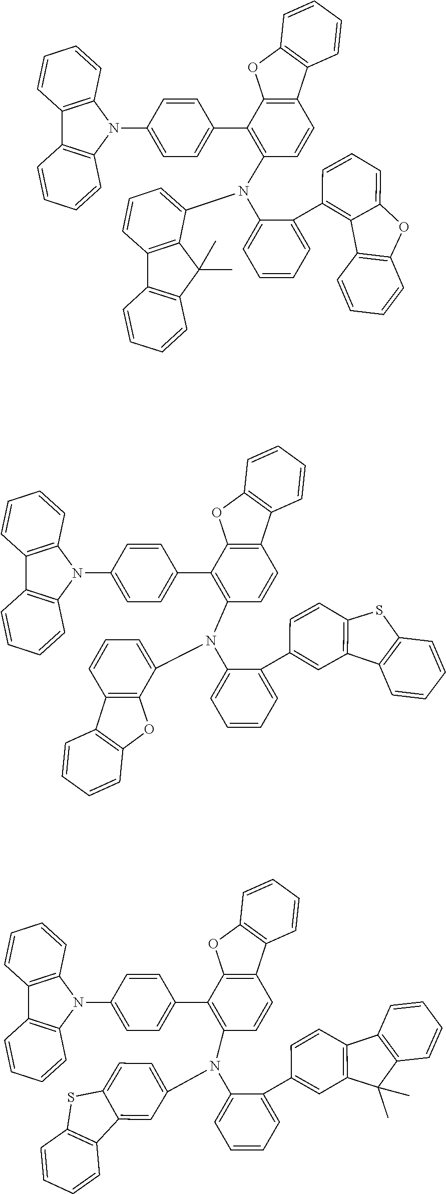 [SMILES: CC1(C)c2ccccc2-c2cccc(N(c3ccccc3-c3cccc4oc5ccccc5c34)c3ccc4c(oc5ccccc54)c3-c3ccc(-n4c5ccccc5c5ccccc54)cc3)c21 ; c1ccc(N(c2ccc3c(oc4ccccc43)c2-c2ccc(-n3c4ccccc4c4ccccc43)cc2)c2cccc3c2oc2ccccc23)c(-c2ccc3sc4ccccc4c3c2)c1 ; CC1(C)c2ccccc2-c2ccc(-c3ccccc3N(c3ccc4sc5ccccc5c4c3)c3ccc4c(oc5ccccc54)c3-c3ccc(-n4c5ccccc5c5ccccc54)cc3)cc21]

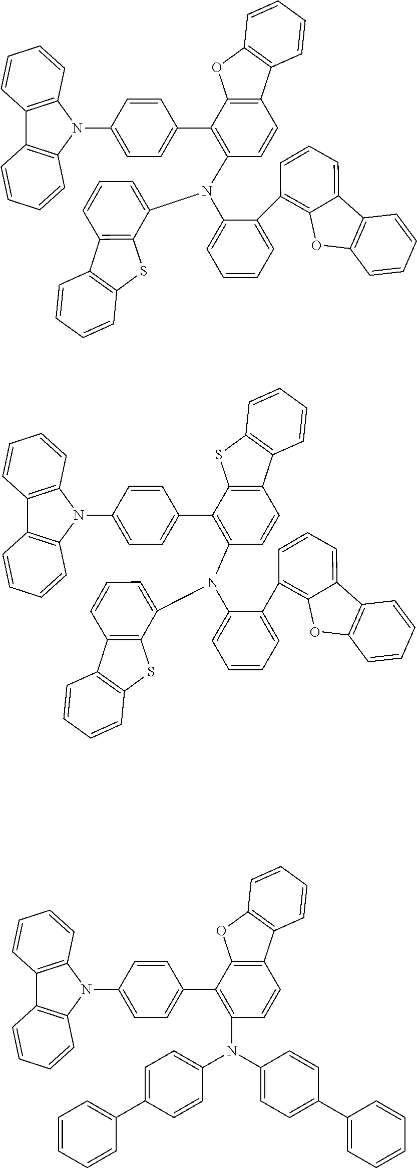 [SMILES: c1ccc(N(c2ccc3c(oc4ccccc43)c2-c2ccc(-n3c4ccccc4c4ccccc43)cc2)c2cccc3c2sc2ccccc23)c(-c2cccc3c2oc2ccccc23)c1 ; c1ccc(N(c2ccc3c(sc4ccccc43)c2-c2ccc(-n3c4ccccc4c4ccccc43)cc2)c2cccc3c2sc2ccccc23)c(-c2cccc3c2oc2ccccc23)c1 ; c1ccc(-c2ccc(N(c3ccc(-c4ccccc4)cc3)c3ccc4c(oc5ccccc54)c3-c3ccc(-n4c5ccccc5c5ccccc54)cc3)cc2)cc1]

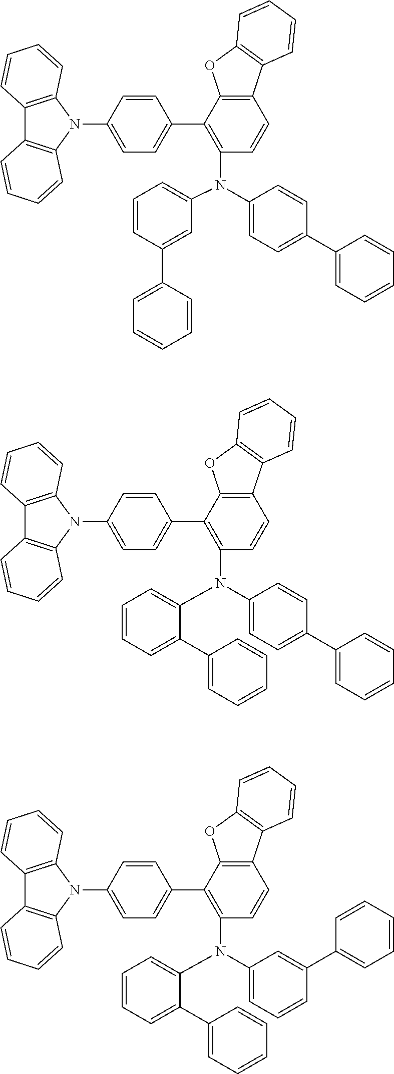 [SMILES: c1ccc(-c2ccc(N(c3cccc(-c4ccccc4)c3)c3ccc4c(oc5ccccc54)c3-c3ccc(-n4c5ccccc5c5ccccc54)cc3)cc2)cc1 ; c1ccc(-c2ccc(N(c3ccccc3-c3ccccc3)c3ccc4c(oc5ccccc54)c3-c3ccc(-n4c5ccccc5c5ccccc54)cc3)cc2)cc1 ; c1ccc(-c2cccc(N(c3ccccc3-c3ccccc3)c3ccc4c(oc5ccccc54)c3-c3ccc(-n4c5ccccc5c5ccccc54)cc3)c2)cc1]

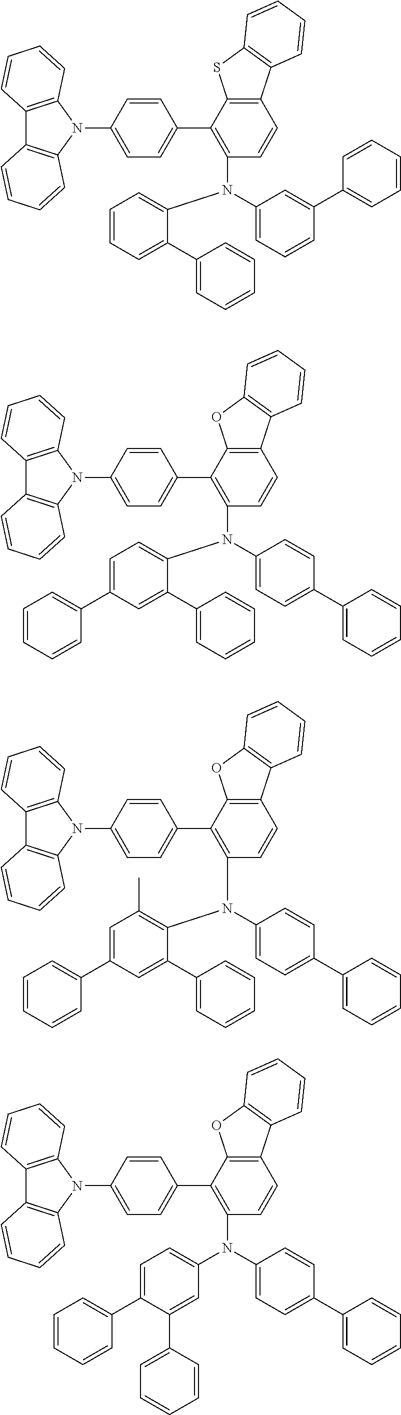 [SMILES: c1ccc(-c2cccc(N(c3ccccc3-c3ccccc3)c3ccc4c(sc5ccccc54)c3-c3ccc(-n4c5ccccc5c5ccccc54)cc3)c2)cc1 ; c1ccc(-c2ccc(N(c3ccc(-c4ccccc4)cc3-c3ccccc3)c3ccc4c(oc5ccccc54)c3-c3ccc(-n4c5ccccc5c5ccccc54)cc3)cc2)cc1 ; Cc1cc(-c2ccccc2)cc(-c2ccccc2)c1N(c1ccc(-c2ccccc2)cc1)c1ccc2c(oc3ccccc32)c1-c1ccc(-n2c3ccccc3c3ccccc32)cc1 ; c1ccc(-c2ccc(N(c3ccc(-c4ccccc4)c(-c4ccccc4)c3)c3ccc4c(oc5ccccc54)c3-c3ccc(-n4c5ccccc5c5ccccc54)cc3)cc2)cc1]

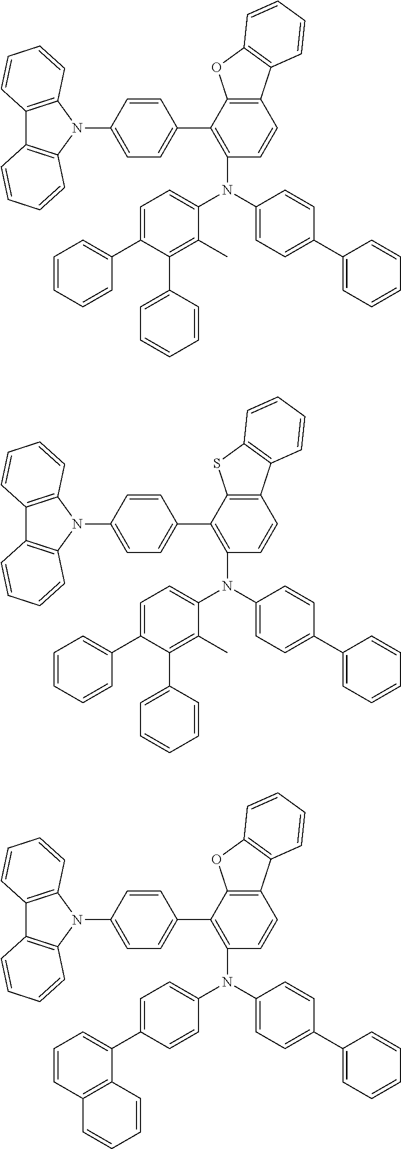 [SMILES: Cc1c(N(c2ccc(-c3ccccc3)cc2)c2ccc3c(oc4ccccc43)c2-c2ccc(-n3c4ccccc4c4ccccc43)cc2)ccc(-c2ccccc2)c1-c1ccccc1 ; Cc1c(N(c2ccc(-c3ccccc3)cc2)c2ccc3c(sc4ccccc43)c2-c2ccc(-n3c4ccccc4c4ccccc43)cc2)ccc(-c2ccccc2)c1-c1ccccc1 ; c1ccc(-c2ccc(N(c3ccc(-c4cccc5ccccc45)cc3)c3ccc4c(oc5ccccc54)c3-c3ccc(-n4c5ccccc5c5ccccc54)cc3)cc2)cc1]

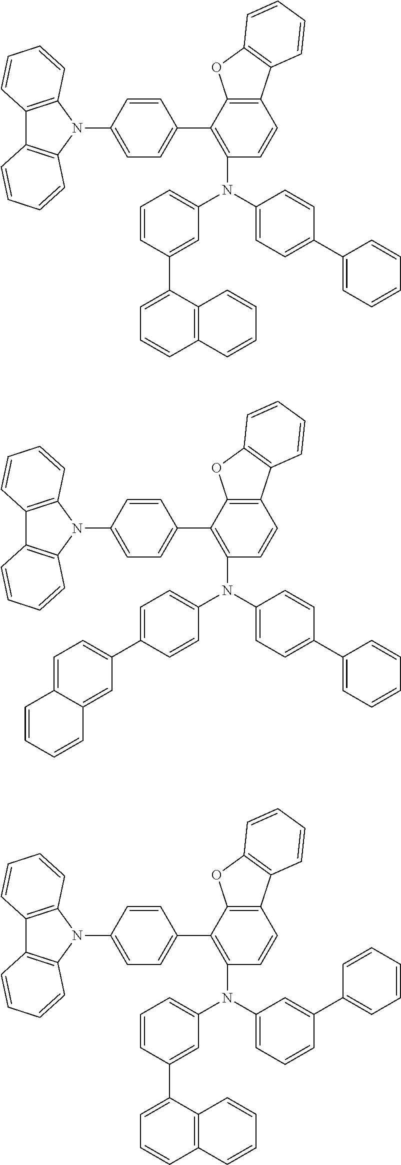 [SMILES: c1ccc(-c2ccc(N(c3cccc(-c4cccc5ccccc45)c3)c3ccc4c(oc5ccccc54)c3-c3ccc(-n4c5ccccc5c5ccccc54)cc3)cc2)cc1 ; c1ccc(-c2ccc(N(c3ccc(-c4ccc5ccccc5c4)cc3)c3ccc4c(oc5ccccc54)c3-c3ccc(-n4c5ccccc5c5ccccc54)cc3)cc2)cc1 ; c1ccc(-c2cccc(N(c3cccc(-c4cccc5ccccc45)c3)c3ccc4c(oc5ccccc54)c3-c3ccc(-n4c5ccccc5c5ccccc54)cc3)c2)cc1]

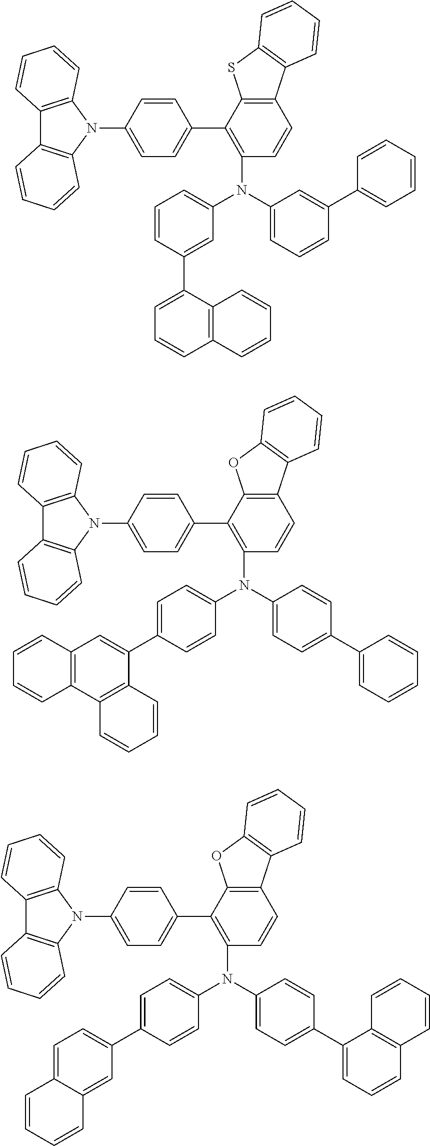 [SMILES: c1ccc(-c2cccc(N(c3cccc(-c4cccc5ccccc45)c3)c3ccc4c(sc5ccccc54)c3-c3ccc(-n4c5ccccc5c5ccccc54)cc3)c2)cc1 ; c1ccc(-c2ccc(N(c3ccc(-c4cc5ccccc5c5ccccc45)cc3)c3ccc4c(oc5ccccc54)c3-c3ccc(-n4c5ccccc5c5ccccc54)cc3)cc2)cc1 ; c1ccc2cc(-c3ccc(N(c4ccc(-c5cccc6ccccc56)cc4)c4ccc5c(oc6ccccc65)c4-c4ccc(-n5c6ccccc6c6ccccc65)cc4)cc3)ccc2c1]

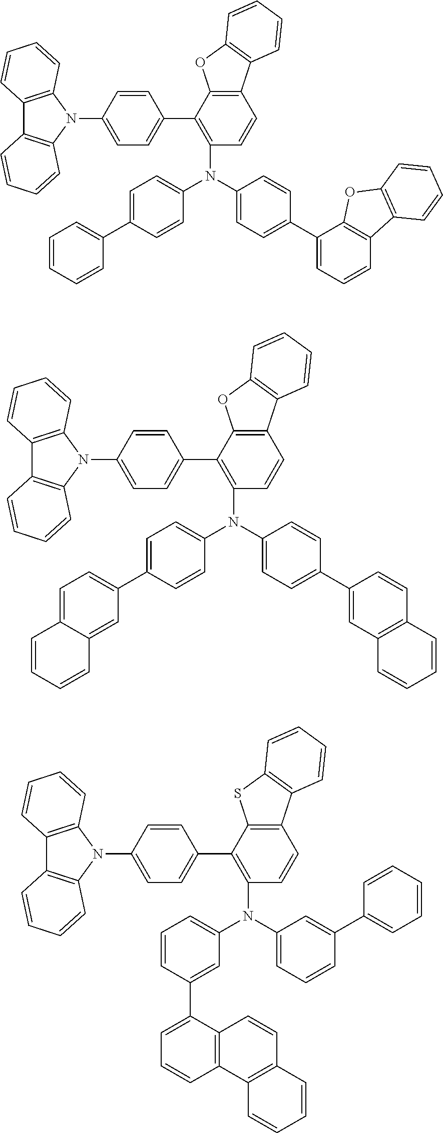 [SMILES: c1ccc(-c2ccc(N(c3ccc(-c4cccc5c4oc4ccccc45)cc3)c3ccc4c(oc5ccccc54)c3-c3ccc(-n4c5ccccc5c5ccccc54)cc3)cc2)cc1 ; c1ccc2cc(-c3ccc(N(c4ccc(-c5ccc6ccccc6c5)cc4)c4ccc5c(oc6ccccc65)c4-c4ccc(-n5c6ccccc6c6ccccc65)cc4)cc3)ccc2c1 ; c1ccc(-c2cccc(N(c3cccc(-c4cccc5c4ccc4ccccc45)c3)c3ccc4c(sc5ccccc54)c3-c3ccc(-n4c5ccccc5c5ccccc54)cc3)c2)cc1]

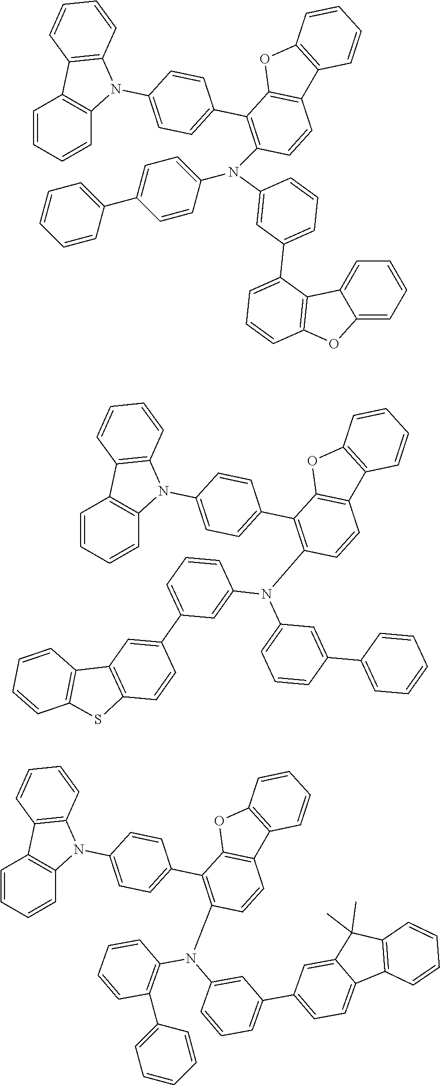 [SMILES: c1ccc(-c2ccc(N(c3cccc(-c4cccc5oc6ccccc6c45)c3)c3ccc4c(oc5ccccc54)c3-c3ccc(-n4c5ccccc5c5ccccc54)cc3)cc2)cc1 ; c1ccc(-c2cccc(N(c3cccc(-c4ccc5sc6ccccc6c5c4)c3)c3ccc4c(oc5ccccc54)c3-c3ccc(-n4c5ccccc5c5ccccc54)cc3)c2)cc1 ; CC1(C)c2ccccc2-c2ccc(-c3cccc(N(c4ccccc4-c4ccccc4)c4ccc5c(oc6ccccc65)c4-c4ccc(-n5c6ccccc6c6ccccc65)cc4)c3)cc21]

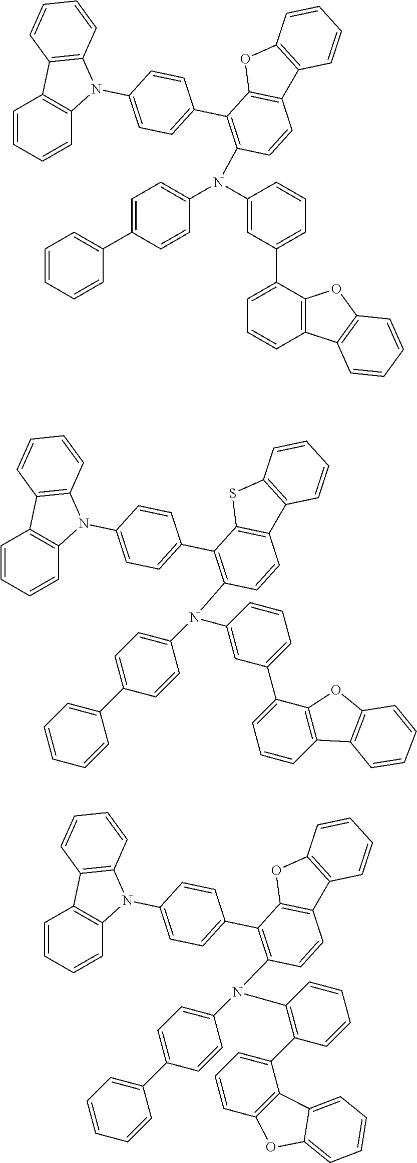 [SMILES: c1ccc(-c2ccc(N(c3cccc(-c4cccc5c4oc4ccccc45)c3)c3ccc4c(oc5ccccc54)c3-c3ccc(-n4c5ccccc5c5ccccc54)cc3)cc2)cc1 ; c1ccc(-c2ccc(N(c3cccc(-c4cccc5c4oc4ccccc45)c3)c3ccc4c(sc5ccccc54)c3-c3ccc(-n4c5ccccc5c5ccccc54)cc3)cc2)cc1 ; c1ccc(-c2ccc(N(c3ccccc3-c3cccc4oc5ccccc5c34)c3ccc4c(oc5ccccc54)c3-c3ccc(-n4c5ccccc5c5ccccc54)cc3)cc2)cc1]

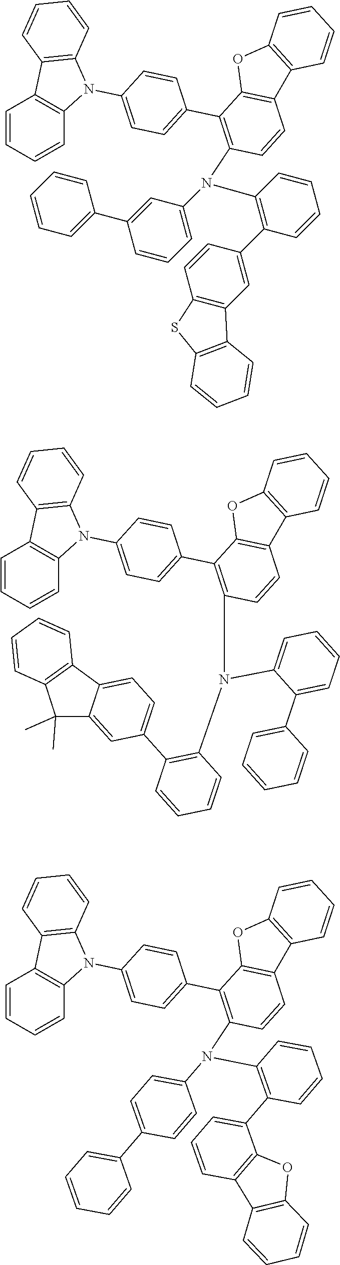 [SMILES: c1ccc(-c2cccc(N(c3ccccc3-c3ccc4sc5ccccc5c4c3)c3ccc4c(oc5ccccc54)c3-c3ccc(-n4c5ccccc5c5ccccc54)cc3)c2)cc1 ; CC1(C)c2ccccc2-c2ccc(-c3ccccc3N(c3ccccc3-c3ccccc3)c3ccc4c(oc5ccccc54)c3-c3ccc(-n4c5ccccc5c5ccccc54)cc3)cc21 ; c1ccc(-c2ccc(N(c3ccccc3-c3cccc4c3oc3ccccc34)c3ccc4c(oc5ccccc54)c3-c3ccc(-n4c5ccccc5c5ccccc54)cc3)cc2)cc1]

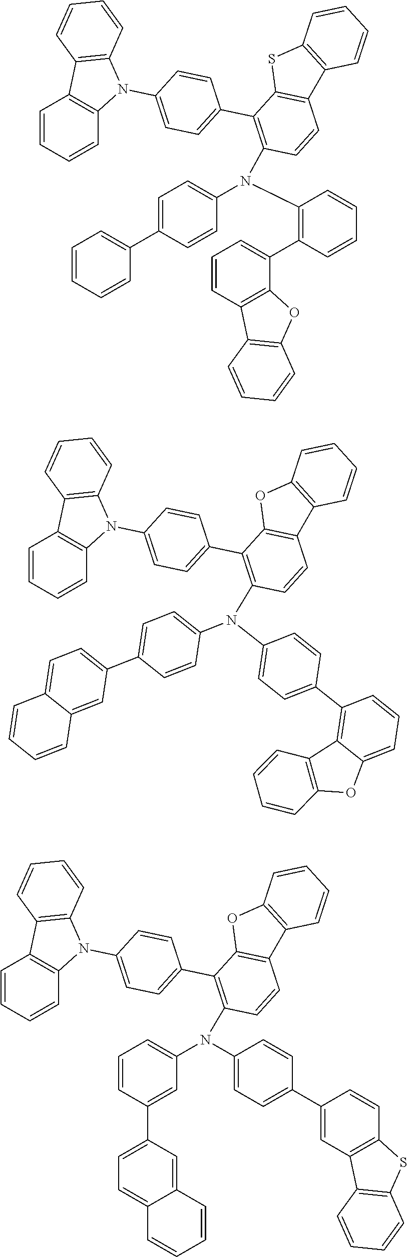 [SMILES: c1ccc(-c2ccc(N(c3ccccc3-c3cccc4c3oc3ccccc34)c3ccc4c(sc5ccccc54)c3-c3ccc(-n4c5ccccc5c5ccccc54)cc3)cc2)cc1 ; c1ccc2cc(-c3ccc(N(c4ccc(-c5cccc6oc7ccccc7c56)cc4)c4ccc5c(oc6ccccc65)c4-c4ccc(-n5c6ccccc6c6ccccc65)cc4)cc3)ccc2c1 ; c1cc(-c2ccc3ccccc3c2)cc(N(c2ccc(-c3ccc4sc5ccccc5c4c3)cc2)c2ccc3c(oc4ccccc43)c2-c2ccc(-n3c4ccccc4c4ccccc43)cc2)c1]

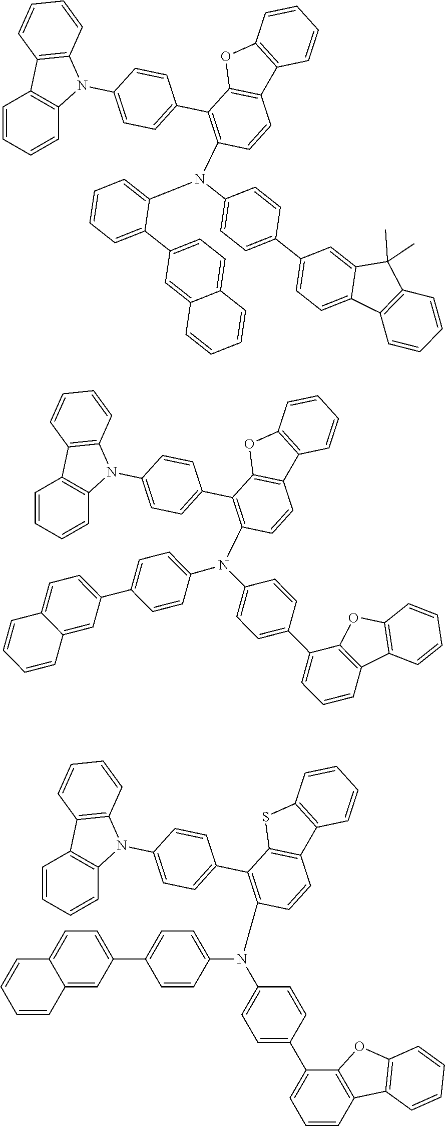 [SMILES: CC1(C)c2ccccc2-c2ccc(-c3ccc(N(c4ccccc4-c4ccc5ccccc5c4)c4ccc5c(oc6ccccc65)c4-c4ccc(-n5c6ccccc6c6ccccc65)cc4)cc3)cc21 ; c1ccc2cc(-c3ccc(N(c4ccc(-c5cccc6c5oc5ccccc56)cc4)c4ccc5c(oc6ccccc65)c4-c4ccc(-n5c6ccccc6c6ccccc65)cc4)cc3)ccc2c1 ; c1ccc2cc(-c3ccc(N(c4ccc(-c5cccc6c5oc5ccccc56)cc4)c4ccc5c(sc6ccccc65)c4-c4ccc(-n5c6ccccc6c6ccccc65)cc4)cc3)ccc2c1]

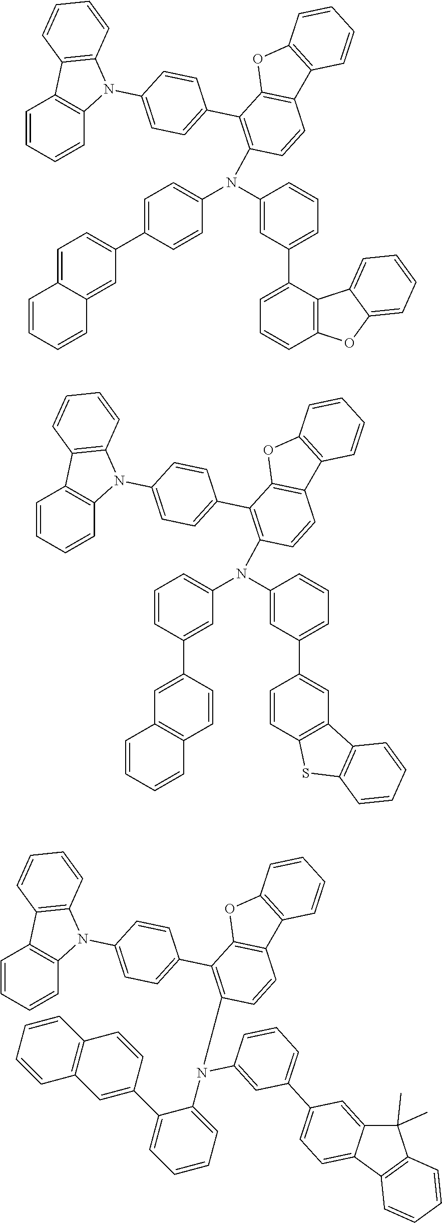 [SMILES: c1cc(-c2cccc3oc4ccccc4c23)cc(N(c2ccc(-c3ccc4ccccc4c3)cc2)c2ccc3c(oc4ccccc43)c2-c2ccc(-n3c4ccccc4c4ccccc43)cc2)c1 ; c1cc(-c2ccc3ccccc3c2)cc(N(c2cccc(-c3ccc4sc5ccccc5c4c3)c2)c2ccc3c(oc4ccccc43)c2-c2ccc(-n3c4ccccc4c4ccccc43)cc2)c1 ; CC1(C)c2ccccc2-c2ccc(-c3cccc(N(c4ccccc4-c4ccc5ccccc5c4)c4ccc5c(oc6ccccc65)c4-c4ccc(-n5c6ccccc6c6ccccc65)cc4)c3)cc21]

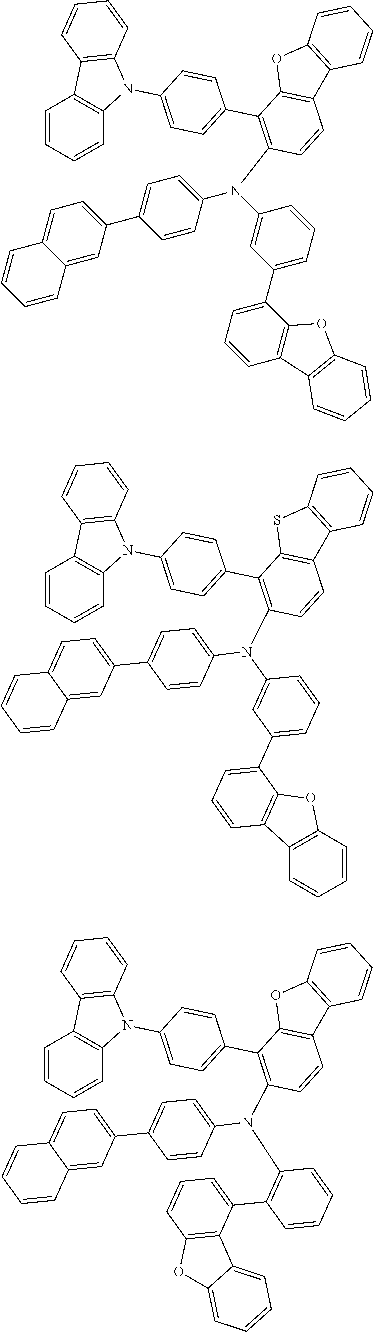 [SMILES: c1cc(-c2cccc3c2oc2ccccc23)cc(N(c2ccc(-c3ccc4ccccc4c3)cc2)c2ccc3c(oc4ccccc43)c2-c2ccc(-n3c4ccccc4c4ccccc43)cc2)c1 ; c1cc(-c2cccc3c2oc2ccccc23)cc(N(c2ccc(-c3ccc4ccccc4c3)cc2)c2ccc3c(sc4ccccc43)c2-c2ccc(-n3c4ccccc4c4ccccc43)cc2)c1 ; c1ccc(N(c2ccc(-c3ccc4ccccc4c3)cc2)c2ccc3c(oc4ccccc43)c2-c2ccc(-n3c4ccccc4c4ccccc43)cc2)c(-c2cccc3oc4ccccc4c23)c1]

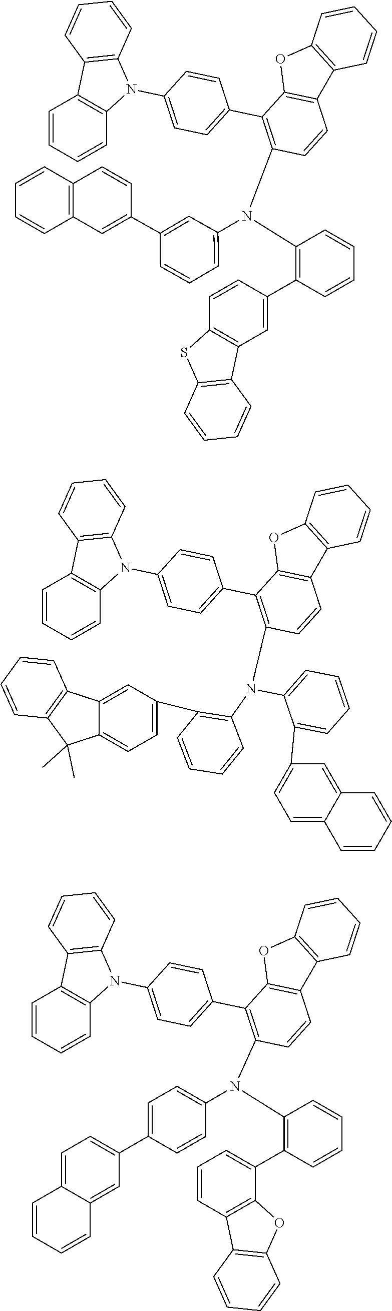 [SMILES: c1cc(-c2ccc3ccccc3c2)cc(N(c2ccccc2-c2ccc3sc4ccccc4c3c2)c2ccc3c(oc4ccccc43)c2-c2ccc(-n3c4ccccc4c4ccccc43)cc2)c1 ; CC1(C)c2ccccc2-c2cc(-c3ccccc3N(c3ccccc3-c3ccc4ccccc4c3)c3ccc4c(oc5ccccc54)c3-c3ccc(-n4c5ccccc5c5ccccc54)cc3)ccc21 ; c1ccc(N(c2ccc(-c3ccc4ccccc4c3)cc2)c2ccc3c(oc4ccccc43)c2-c2ccc(-n3c4ccccc4c4ccccc43)cc2)c(-c2cccc3c2oc2ccccc23)c1]

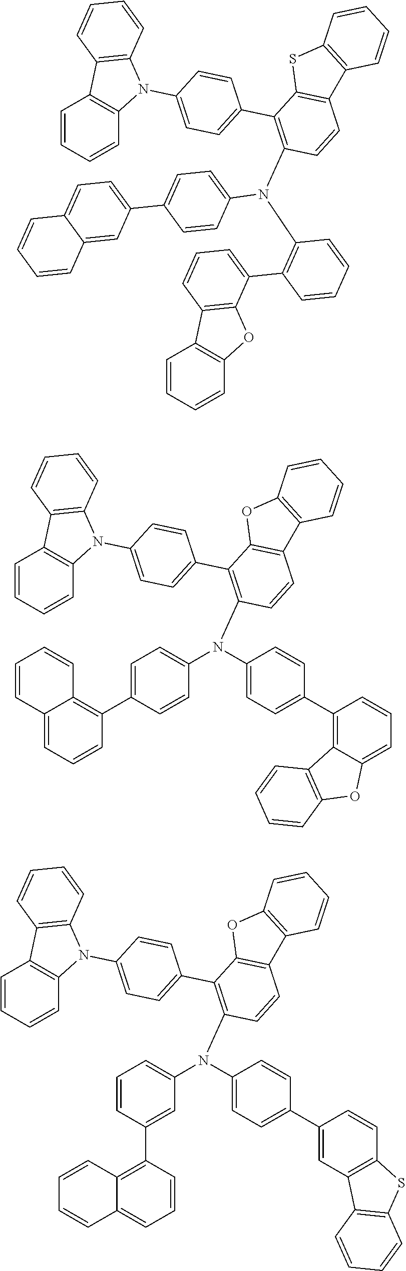

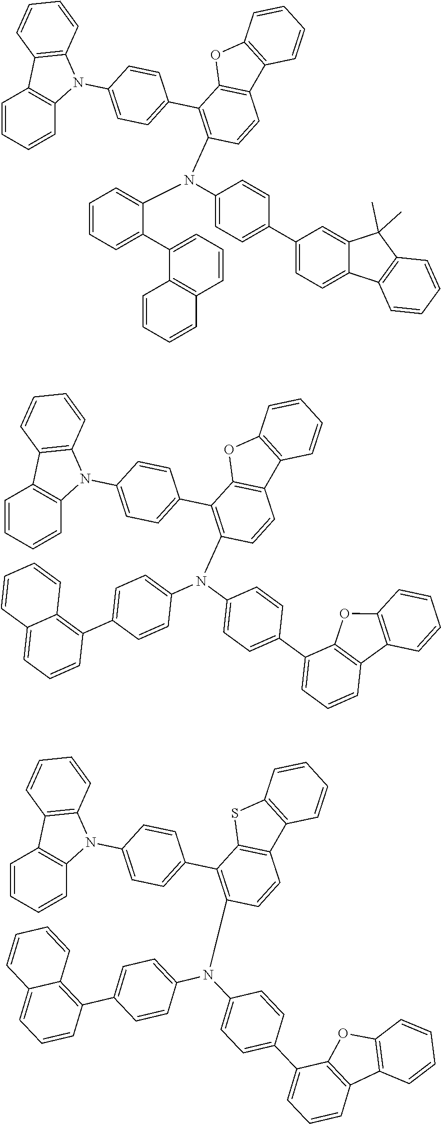 [SMILES: CC1(C)c2ccccc2-c2ccc(-c3ccc(N(c4ccccc4-c4cccc5ccccc45)c4ccc5c(oc6ccccc65)c4-c4ccc(-n5c6ccccc6c6ccccc65)cc4)cc3)cc21 ; c1ccc2c(-c3ccc(N(c4ccc(-c5cccc6c5oc5ccccc56)cc4)c4ccc5c(oc6ccccc65)c4-c4ccc(-n5c6ccccc6c6ccccc65)cc4)cc3)cccc2c1 ; c1ccc2c(-c3ccc(N(c4ccc(-c5cccc6c5oc5ccccc56)cc4)c4ccc5c(sc6ccccc65)c4-c4ccc(-n5c6ccccc6c6ccccc65)cc4)cc3)cccc2c1]

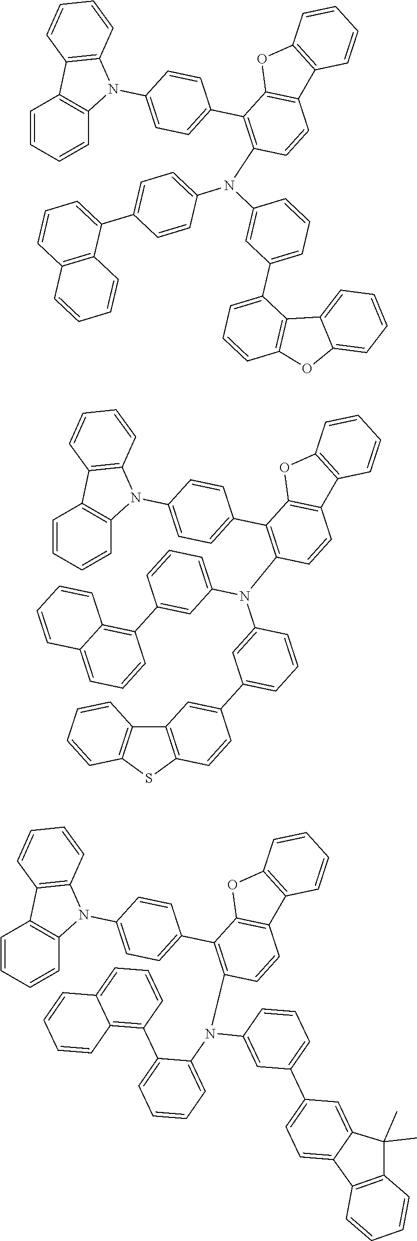 [SMILES: c1cc(-c2cccc3oc4ccccc4c23)cc(N(c2ccc(-c3cccc4ccccc34)cc2)c2ccc3c(oc4ccccc43)c2-c2ccc(-n3c4ccccc4c4ccccc43)cc2)c1 ; c1cc(-c2ccc3sc4ccccc4c3c2)cc(N(c2cccc(-c3cccc4ccccc34)c2)c2ccc3c(oc4ccccc43)c2-c2ccc(-n3c4ccccc4c4ccccc43)cc2)c1 ; CC1(C)c2ccccc2-c2ccc(-c3cccc(N(c4ccccc4-c4cccc5ccccc45)c4ccc5c(oc6ccccc65)c4-c4ccc(-n5c6ccccc6c6ccccc65)cc4)c3)cc21]

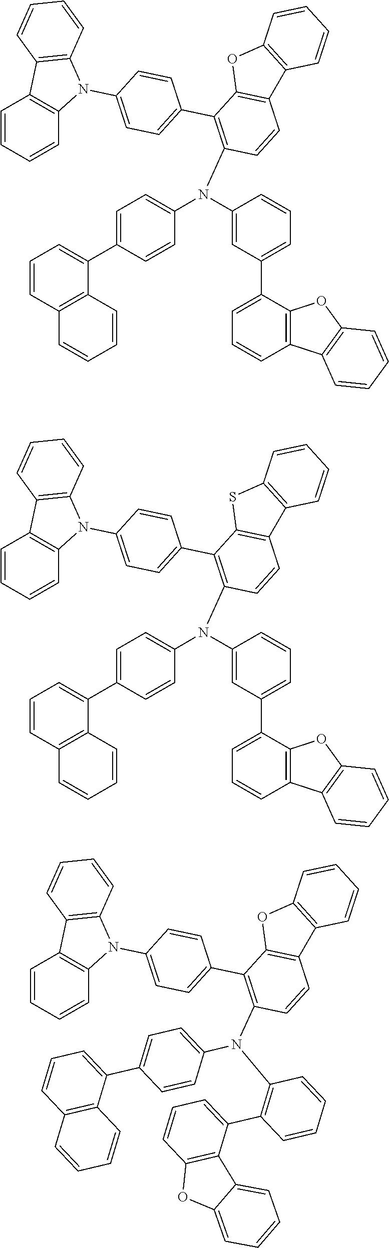

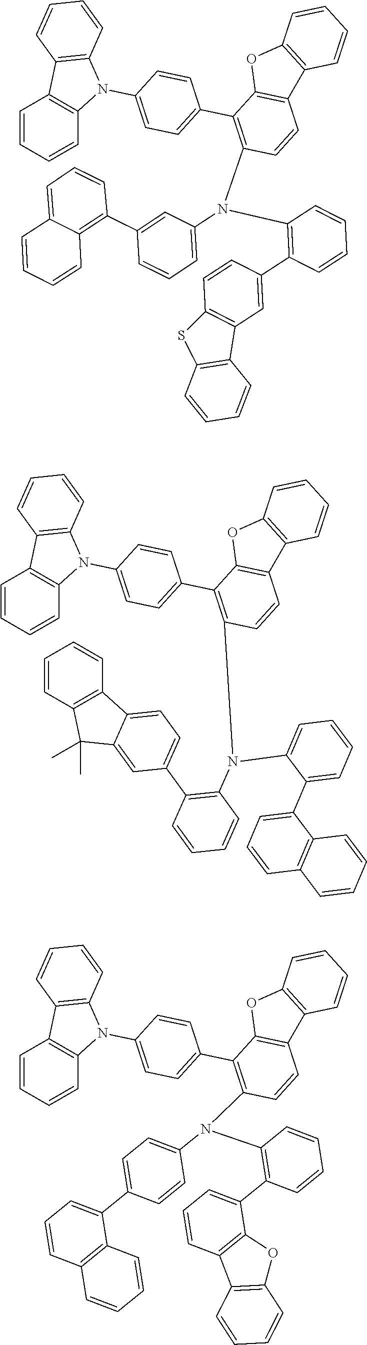 [SMILES: c1cc(-c2cccc3ccccc23)cc(N(c2ccccc2-c2ccc3sc4ccccc4c3c2)c2ccc3c(oc4ccccc43)c2-c2ccc(-n3c4ccccc4c4ccccc43)cc2)c1 ; CC1(C)c2ccccc2-c2ccc(-c3ccccc3N(c3ccccc3-c3cccc4ccccc34)c3ccc4c(oc5ccccc54)c3-c3ccc(-n4c5ccccc5c5ccccc54)cc3)cc21 ; c1ccc(N(c2ccc(-c3cccc4ccccc34)cc2)c2ccc3c(oc4ccccc43)c2-c2ccc(-n3c4ccccc4c4ccccc43)cc2)c(-c2cccc3c2oc2ccccc23)c1]

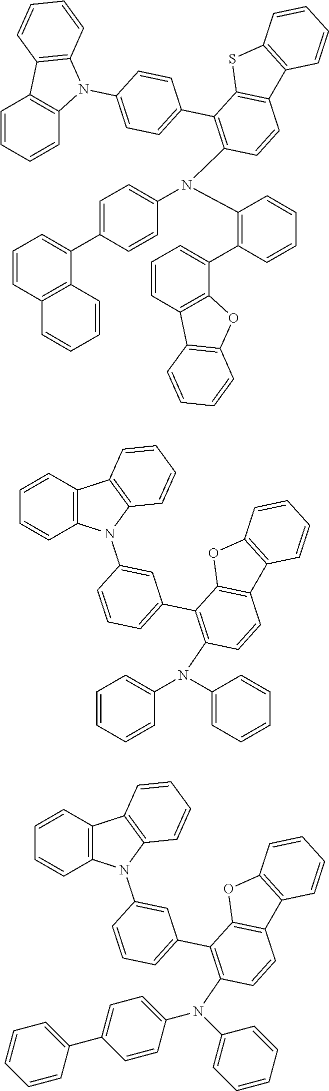

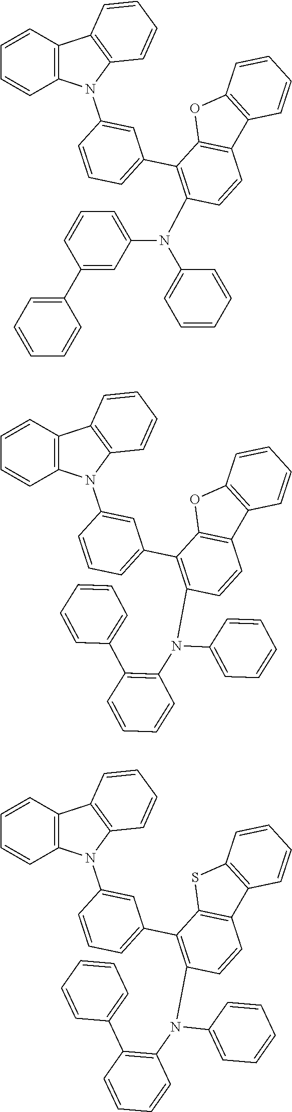

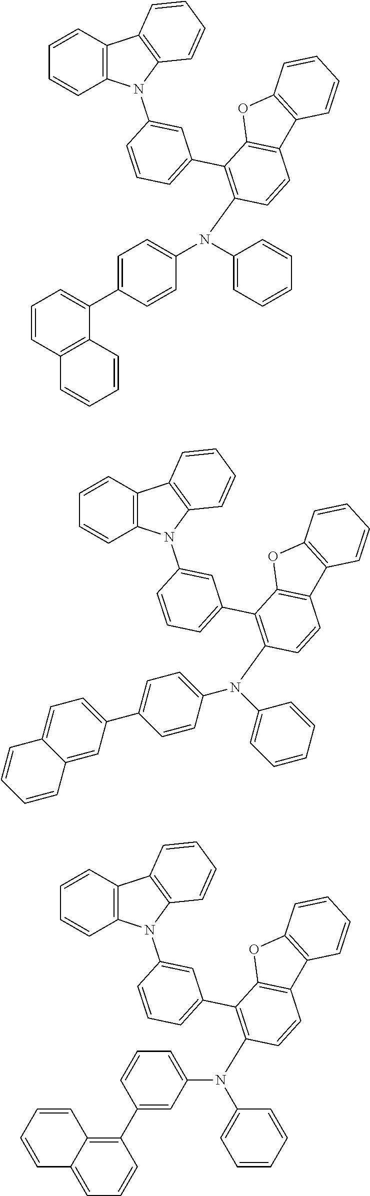 [SMILES: c1ccc(N(c2ccc(-c3cccc4ccccc34)cc2)c2ccc3c(oc4ccccc43)c2-c2cccc(-n3c4ccccc4c4ccccc43)c2)cc1 ; c1ccc(N(c2ccc(-c3ccc4ccccc4c3)cc2)c2ccc3c(oc4ccccc43)c2-c2cccc(-n3c4ccccc4c4ccccc43)c2)cc1 ; c1ccc(N(c2cccc(-c3cccc4ccccc34)c2)c2ccc3c(oc4ccccc43)c2-c2cccc(-n3c4ccccc4c4ccccc43)c2)cc1]

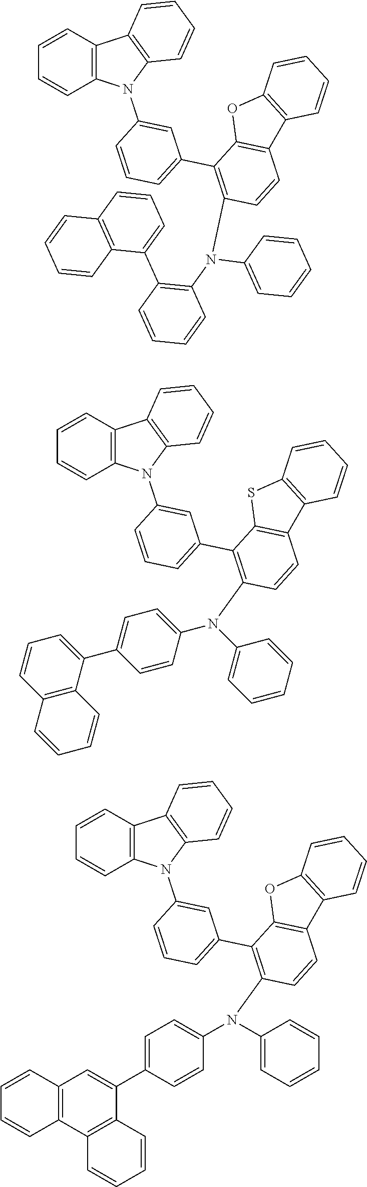

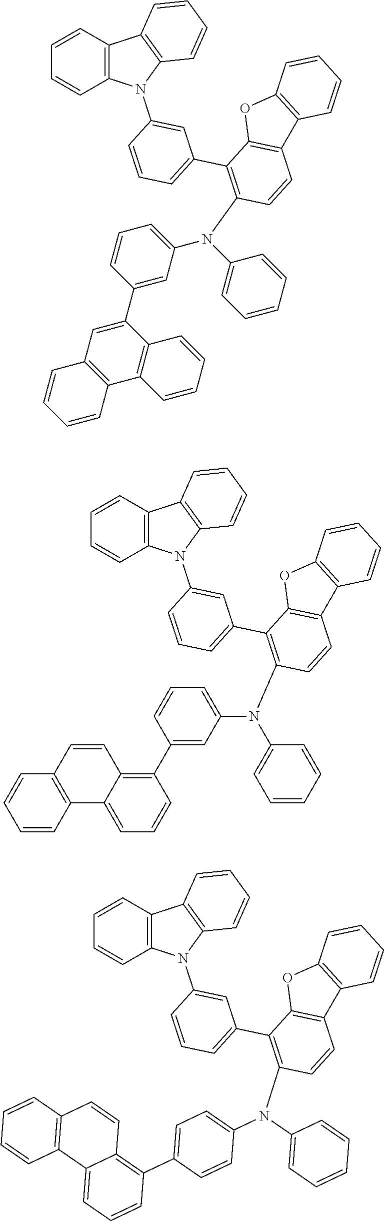 [SMILES: c1ccc(N(c2cccc(-c3cc4ccccc4c4ccccc34)c2)c2ccc3c(oc4ccccc43)c2-c2cccc(-n3c4ccccc4c4ccccc43)c2)cc1 ; c1ccc(N(c2cccc(-c3cccc4c3ccc3ccccc34)c2)c2ccc3c(oc4ccccc43)c2-c2cccc(-n3c4ccccc4c4ccccc43)c2)cc1 ; c1ccc(N(c2ccc(-c3cccc4c3ccc3ccccc34)cc2)c2ccc3c(oc4ccccc43)c2-c2cccc(-n3c4ccccc4c4ccccc43)c2)cc1]

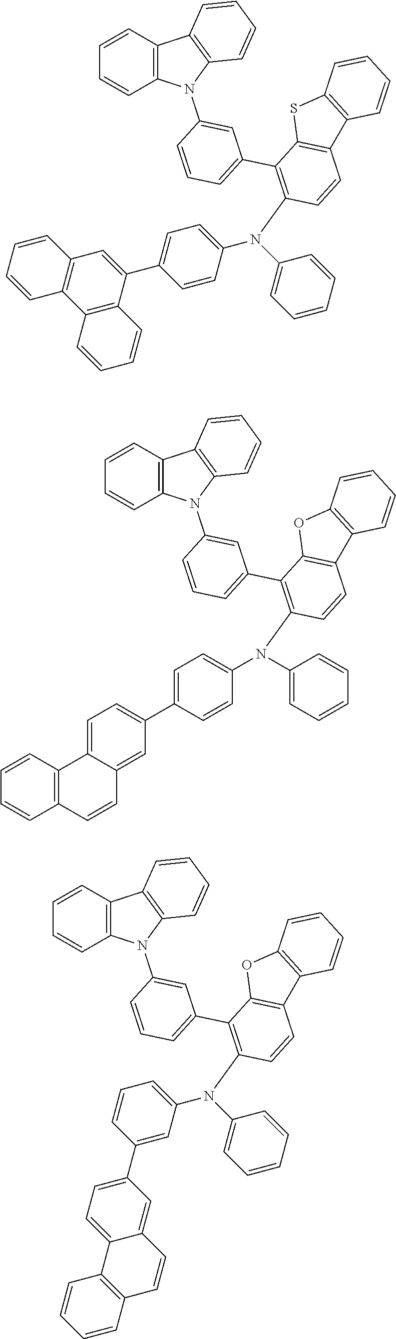 [SMILES: c1ccc(N(c2ccc(-c3cc4ccccc4c4ccccc34)cc2)c2ccc3c(sc4ccccc43)c2-c2cccc(-n3c4ccccc4c4ccccc43)c2)cc1 ; c1ccc(N(c2ccc(-c3ccc4c(ccc5ccccc54)c3)cc2)c2ccc3c(oc4ccccc43)c2-c2cccc(-n3c4ccccc4c4ccccc43)c2)cc1 ; c1ccc(N(c2cccc(-c3ccc4c(ccc5ccccc54)c3)c2)c2ccc3c(oc4ccccc43)c2-c2cccc(-n3c4ccccc4c4ccccc43)c2)cc1]

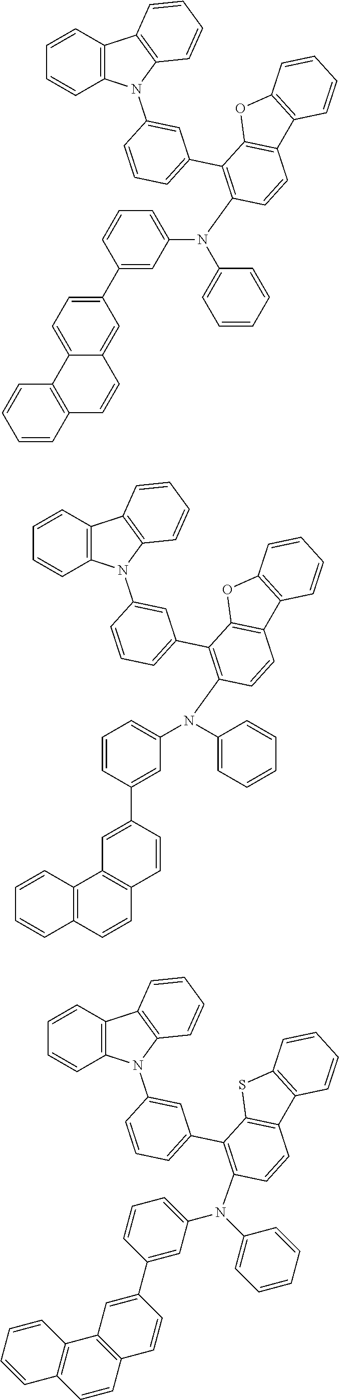 [SMILES: c1ccc(N(c2cccc(-c3ccc4c(ccc5ccccc54)c3)c2)c2ccc3c(oc4ccccc43)c2-c2cccc(-n3c4ccccc4c4ccccc43)c2)cc1 ; c1ccc(N(c2cccc(-c3ccc4ccc5ccccc5c4c3)c2)c2ccc3c(oc4ccccc43)c2-c2cccc(-n3c4ccccc4c4ccccc43)c2)cc1 ; c1ccc(N(c2cccc(-c3ccc4ccc5ccccc5c4c3)c2)c2ccc3c(sc4ccccc43)c2-c2cccc(-n3c4ccccc4c4ccccc43)c2)cc1]

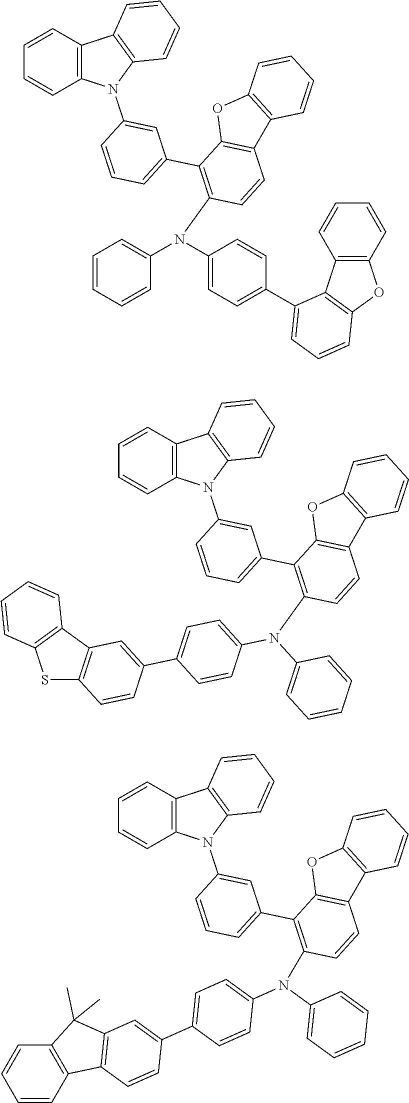 [SMILES: c1ccc(N(c2ccc(-c3cccc4oc5ccccc5c34)cc2)c2ccc3c(oc4ccccc43)c2-c2cccc(-n3c4ccccc4c4ccccc43)c2)cc1 ; c1ccc(N(c2ccc(-c3ccc4sc5ccccc5c4c3)cc2)c2ccc3c(oc4ccccc43)c2-c2cccc(-n3c4ccccc4c4ccccc43)c2)cc1 ; CC1(C)c2ccccc2-c2ccc(-c3ccc(N(c4ccccc4)c4ccc5c(oc6ccccc65)c4-c4cccc(-n5c6ccccc6c6ccccc65)c4)cc3)cc21]

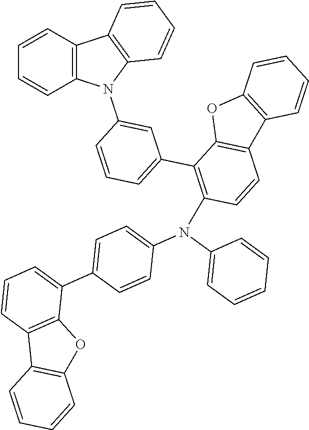 [SMILES: c1ccc(N(c2ccc(-c3cccc4c3oc3ccccc34)cc2)c2ccc3c(oc4ccccc43)c2-c2cccc(-n3c4ccccc4c4ccccc43)c2)cc1]

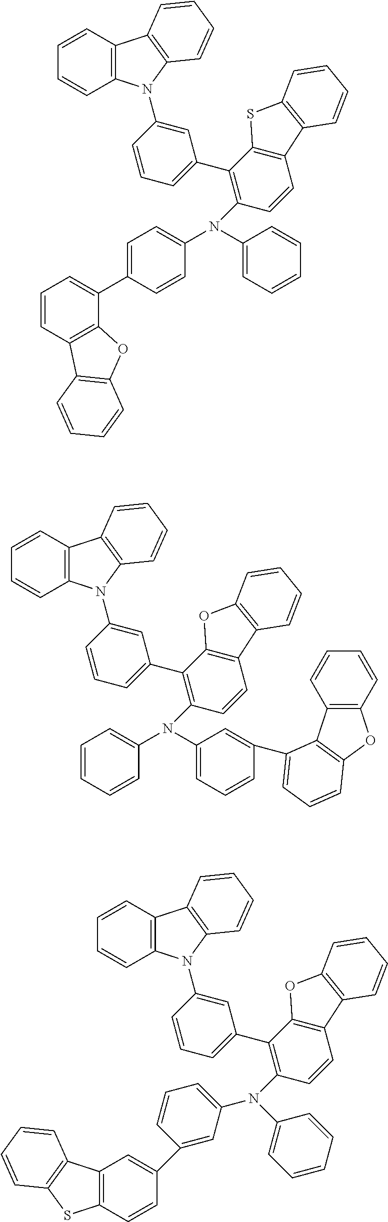 [SMILES: c1ccc(N(c2ccc(-c3cccc4c3oc3ccccc34)cc2)c2ccc3c(sc4ccccc43)c2-c2cccc(-n3c4ccccc4c4ccccc43)c2)cc1 ; c1ccc(N(c2cccc(-c3cccc4oc5ccccc5c34)c2)c2ccc3c(oc4ccccc43)c2-c2cccc(-n3c4ccccc4c4ccccc43)c2)cc1 ; c1ccc(N(c2cccc(-c3ccc4sc5ccccc5c4c3)c2)c2ccc3c(oc4ccccc43)c2-c2cccc(-n3c4ccccc4c4ccccc43)c2)cc1]

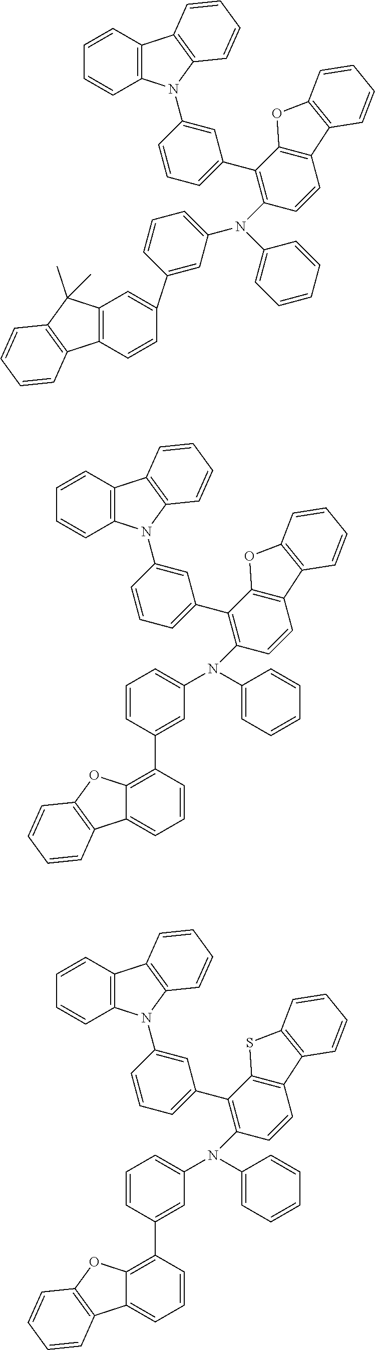 [SMILES: CC1(C)c2ccccc2-c2ccc(-c3cccc(N(c4ccccc4)c4ccc5c(oc6ccccc65)c4-c4cccc(-n5c6ccccc6c6ccccc65)c4)c3)cc21 ; c1ccc(N(c2cccc(-c3cccc4c3oc3ccccc34)c2)c2ccc3c(oc4ccccc43)c2-c2cccc(-n3c4ccccc4c4ccccc43)c2)cc1 ; c1ccc(N(c2cccc(-c3cccc4c3oc3ccccc34)c2)c2ccc3c(sc4ccccc43)c2-c2cccc(-n3c4ccccc4c4ccccc43)c2)cc1]

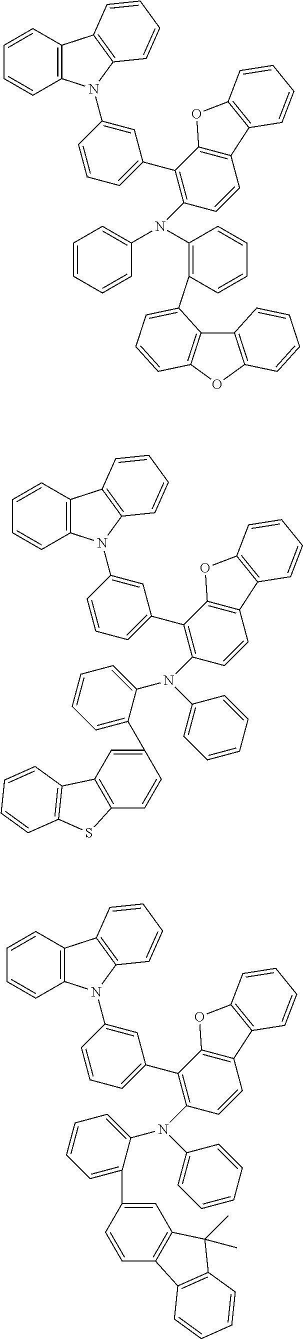 [SMILES: c1ccc(N(c2ccccc2-c2cccc3oc4ccccc4c23)c2ccc3c(oc4ccccc43)c2-c2cccc(-n3c4ccccc4c4ccccc43)c2)cc1 ; c1ccc(N(c2ccccc2-c2ccc3sc4ccccc4c3c2)c2ccc3c(oc4ccccc43)c2-c2cccc(-n3c4ccccc4c4ccccc43)c2)cc1 ; CC1(C)c2ccccc2-c2ccc(-c3ccccc3N(c3ccccc3)c3ccc4c(oc5ccccc54)c3-c3cccc(-n4c5ccccc5c5ccccc54)c3)cc21]

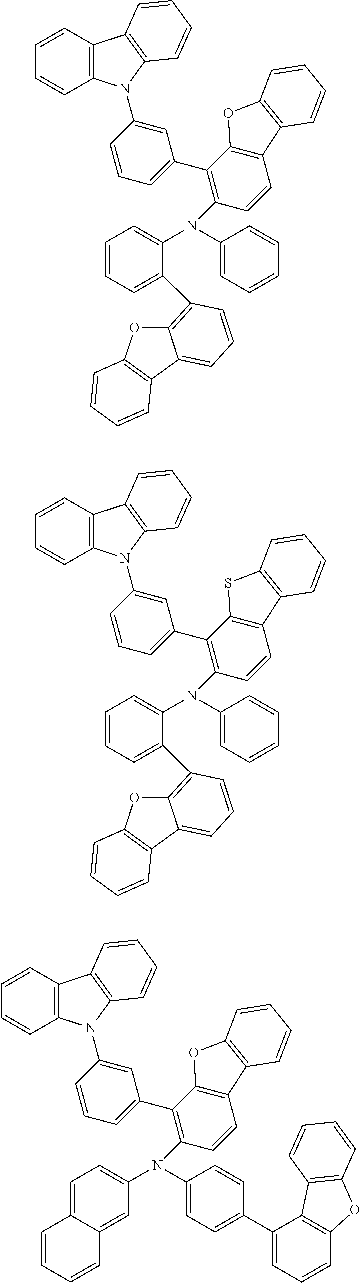 [SMILES: c1ccc(N(c2ccccc2-c2cccc3c2oc2ccccc23)c2ccc3c(oc4ccccc43)c2-c2cccc(-n3c4ccccc4c4ccccc43)c2)cc1 ; c1ccc(N(c2ccccc2-c2cccc3c2oc2ccccc23)c2ccc3c(sc4ccccc43)c2-c2cccc(-n3c4ccccc4c4ccccc43)c2)cc1 ; c1cc(-c2c(N(c3ccc(-c4cccc5oc6ccccc6c45)cc3)c3ccc4ccccc4c3)ccc3c2oc2ccccc23)cc(-n2c3ccccc3c3ccccc32)c1]

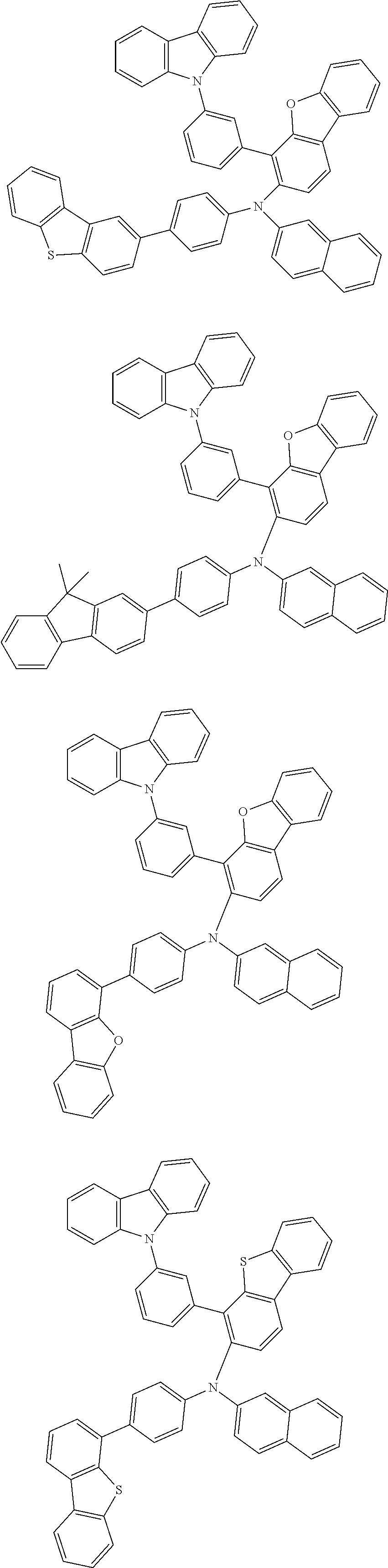 [SMILES: c1cc(-c2c(N(c3ccc(-c4ccc5sc6ccccc6c5c4)cc3)c3ccc4ccccc4c3)ccc3c2oc2ccccc23)cc(-n2c3ccccc3c3ccccc32)c1 ; CC1(C)c2ccccc2-c2ccc(-c3ccc(N(c4ccc5ccccc5c4)c4ccc5c(oc6ccccc65)c4-c4cccc(-n5c6ccccc6c6ccccc65)c4)cc3)cc21 ; c1cc(-c2c(N(c3ccc(-c4cccc5c4oc4ccccc45)cc3)c3ccc4ccccc4c3)ccc3c2oc2ccccc23)cc(-n2c3ccccc3c3ccccc32)c1 ; c1cc(-c2c(N(c3ccc(-c4cccc5c4sc4ccccc45)cc3)c3ccc4ccccc4c3)ccc3c2sc2ccccc23)cc(-n2c3ccccc3c3ccccc32)c1]

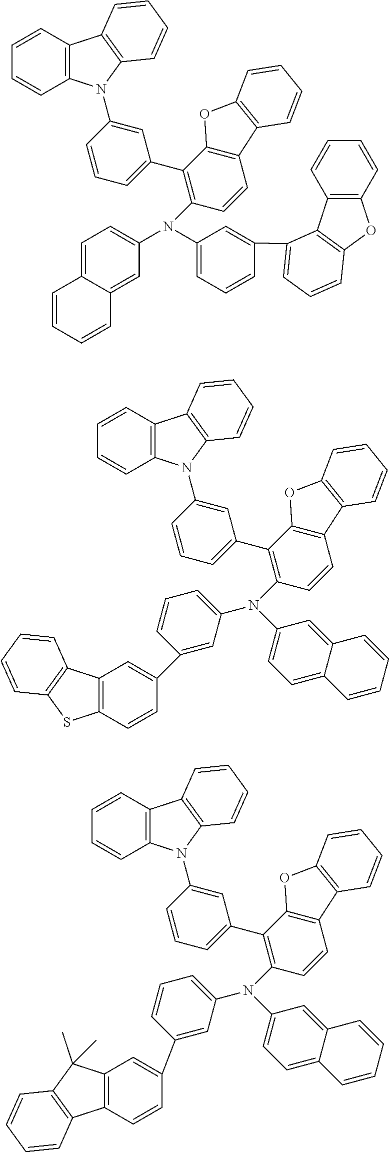 [SMILES: c1cc(-c2cccc3oc4ccccc4c23)cc(N(c2ccc3ccccc3c2)c2ccc3c(oc4ccccc43)c2-c2cccc(-n3c4ccccc4c4ccccc43)c2)c1 ; c1cc(-c2ccc3sc4ccccc4c3c2)cc(N(c2ccc3ccccc3c2)c2ccc3c(oc4ccccc43)c2-c2cccc(-n3c4ccccc4c4ccccc43)c2)c1 ; CC1(C)c2ccccc2-c2ccc(-c3cccc(N(c4ccc5ccccc5c4)c4ccc5c(oc6ccccc65)c4-c4cccc(-n5c6ccccc6c6ccccc65)c4)c3)cc21]

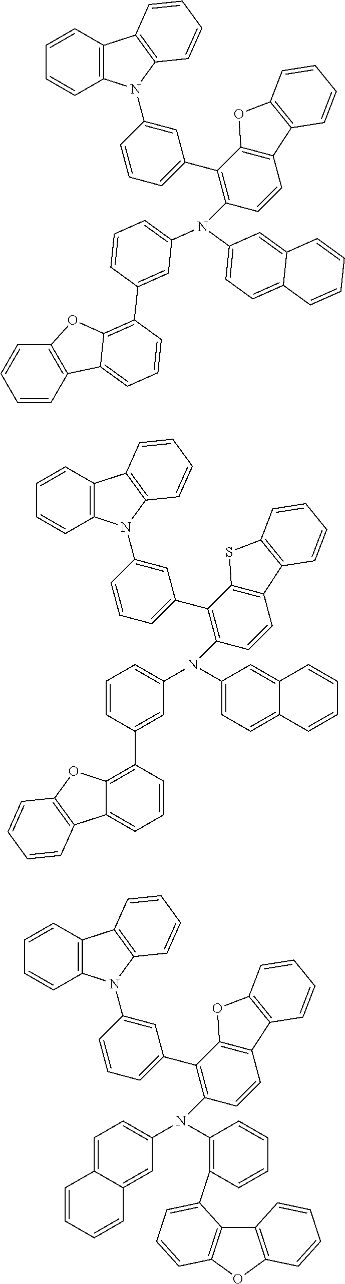 [SMILES: c1cc(-c2cccc3c2oc2ccccc23)cc(N(c2ccc3ccccc3c2)c2ccc3c(oc4ccccc43)c2-c2cccc(-n3c4ccccc4c4ccccc43)c2)c1 ; c1cc(-c2cccc3c2oc2ccccc23)cc(N(c2ccc3ccccc3c2)c2ccc3c(sc4ccccc43)c2-c2cccc(-n3c4ccccc4c4ccccc43)c2)c1 ; c1cc(-c2c(N(c3ccc4ccccc4c3)c3ccccc3-c3cccc4oc5ccccc5c34)ccc3c2oc2ccccc23)cc(-n2c3ccccc3c3ccccc32)c1]

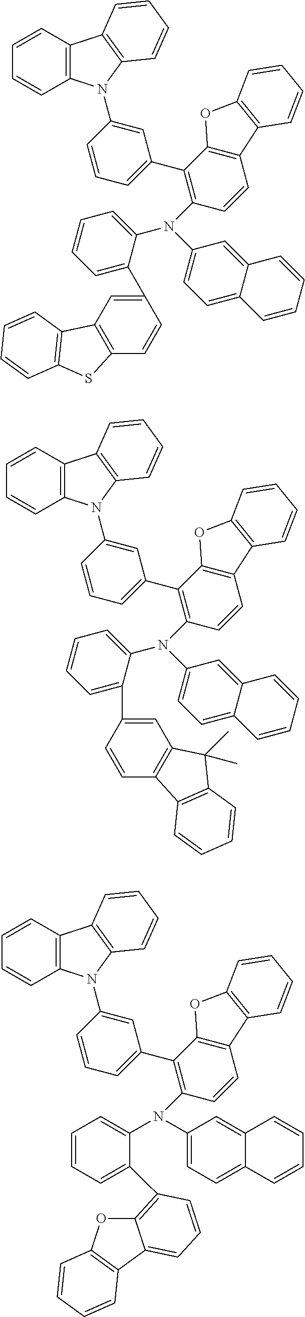 [SMILES: c1cc(-c2c(N(c3ccc4ccccc4c3)c3ccccc3-c3ccc4sc5ccccc5c4c3)ccc3c2oc2ccccc23)cc(-n2c3ccccc3c3ccccc32)c1 ; CC1(C)c2ccccc2-c2ccc(-c3ccccc3N(c3ccc4ccccc4c3)c3ccc4c(oc5ccccc54)c3-c3cccc(-n4c5ccccc5c5ccccc54)c3)cc21 ; c1cc(-c2c(N(c3ccc4ccccc4c3)c3ccccc3-c3cccc4c3oc3ccccc34)ccc3c2oc2ccccc23)cc(-n2c3ccccc3c3ccccc32)c1]

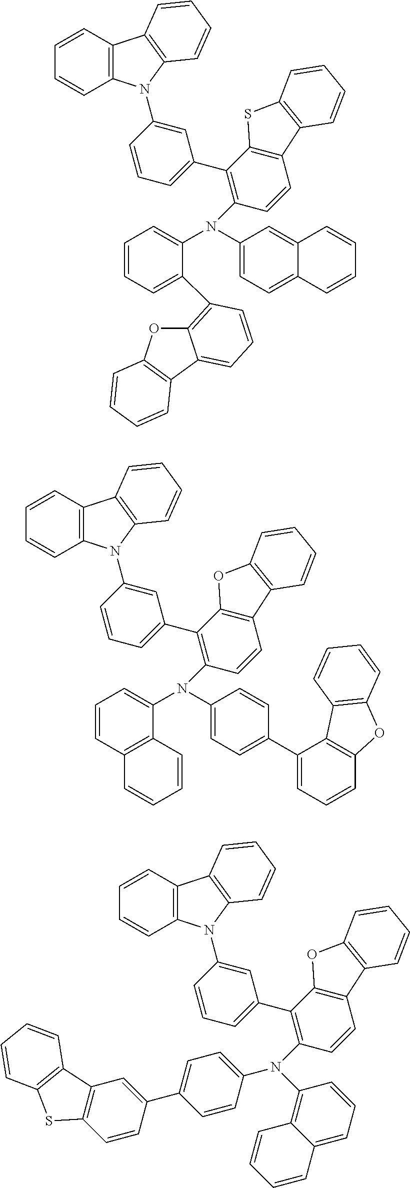

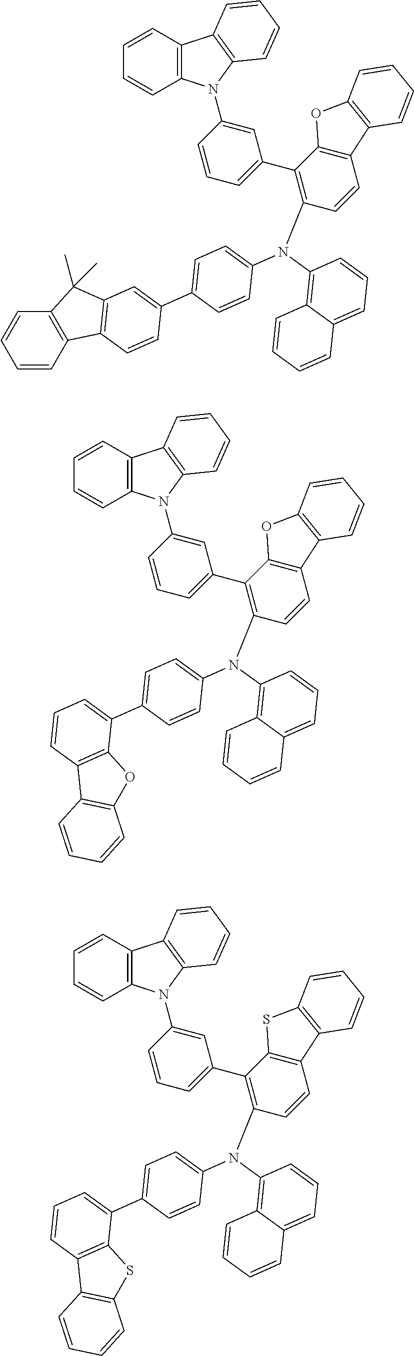 [SMILES: CC1(C)c2ccccc2-c2ccc(-c3ccc(N(c4ccc5c(oc6ccccc65)c4-c4cccc(-n5c6ccccc6c6ccccc65)c4)c4cccc5ccccc45)cc3)cc21 ; c1cc(-c2c(N(c3ccc(-c4cccc5c4oc4ccccc45)cc3)c3cccc4ccccc34)ccc3c2oc2ccccc23)cc(-n2c3ccccc3c3ccccc32)c1 ; c1cc(-c2c(N(c3ccc(-c4cccc5c4sc4ccccc45)cc3)c3cccc4ccccc34)ccc3c2sc2ccccc23)cc(-n2c3ccccc3c3ccccc32)c1]

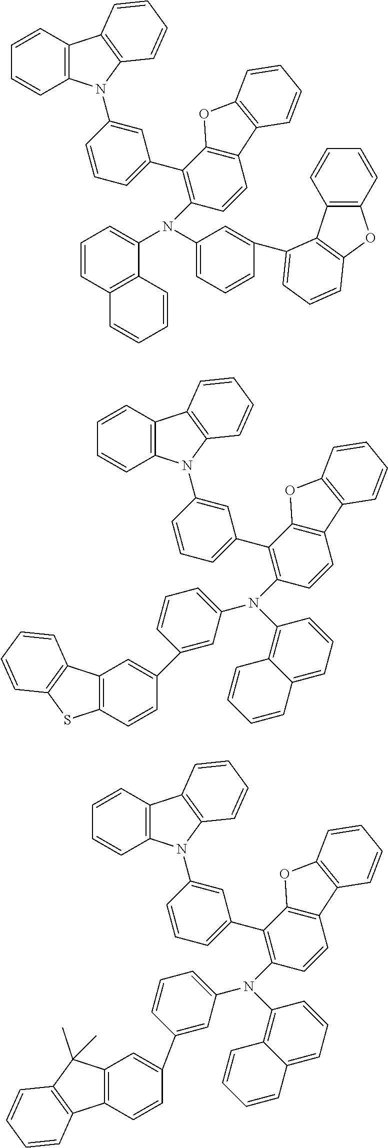

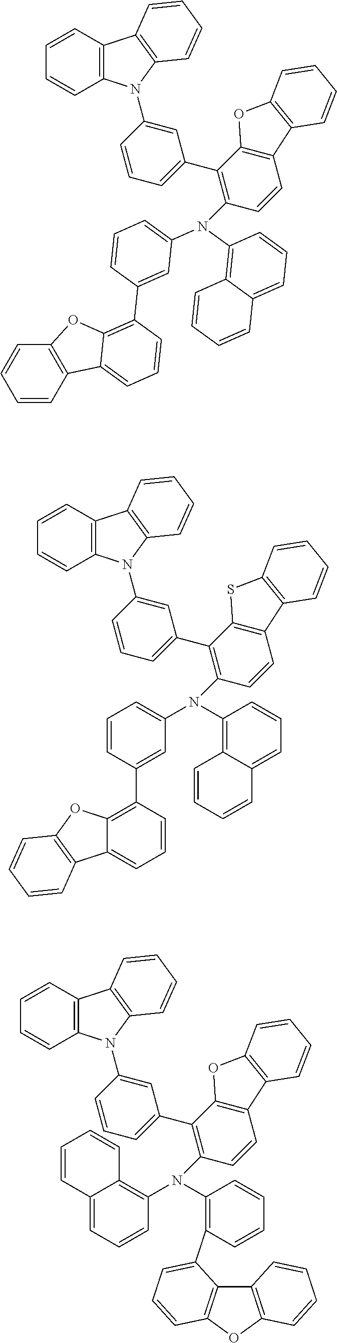 [SMILES: c1cc(-c2cccc3c2oc2ccccc23)cc(N(c2ccc3c(oc4ccccc43)c2-c2cccc(-n3c4ccccc4c4ccccc43)c2)c2cccc3ccccc23)c1 ; c1cc(-c2cccc3c2oc2ccccc23)cc(N(c2ccc3c(sc4ccccc43)c2-c2cccc(-n3c4ccccc4c4ccccc43)c2)c2cccc3ccccc23)c1 ; c1cc(-c2c(N(c3ccccc3-c3cccc4oc5ccccc5c34)c3cccc4ccccc34)ccc3c2oc2ccccc23)cc(-n2c3ccccc3c3ccccc32)c1]

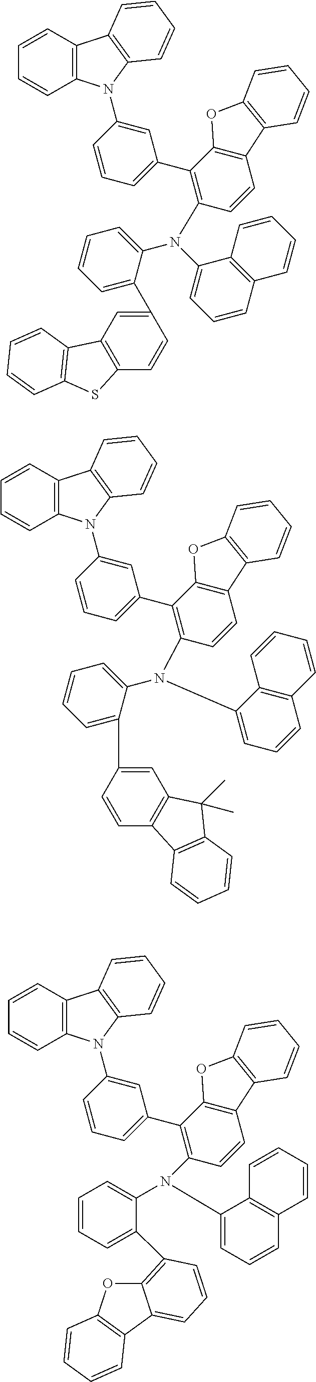

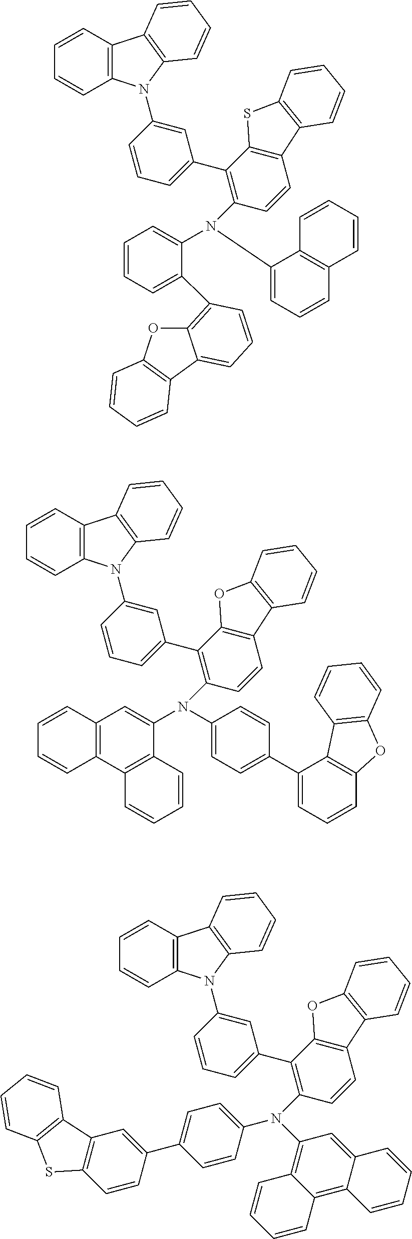 [SMILES: c1cc(-c2c(N(c3ccccc3-c3cccc4c3oc3ccccc34)c3cccc4ccccc34)ccc3c2sc2ccccc23)cc(-n2c3ccccc3c3ccccc32)c1 ; c1cc(-c2c(N(c3ccc(-c4cccc5oc6ccccc6c45)cc3)c3cc4ccccc4c4ccccc34)ccc3c2oc2ccccc23)cc(-n2c3ccccc3c3ccccc32)c1 ; c1cc(-c2c(N(c3ccc(-c4ccc5sc6ccccc6c5c4)cc3)c3cc4ccccc4c4ccccc34)ccc3c2oc2ccccc23)cc(-n2c3ccccc3c3ccccc32)c1]

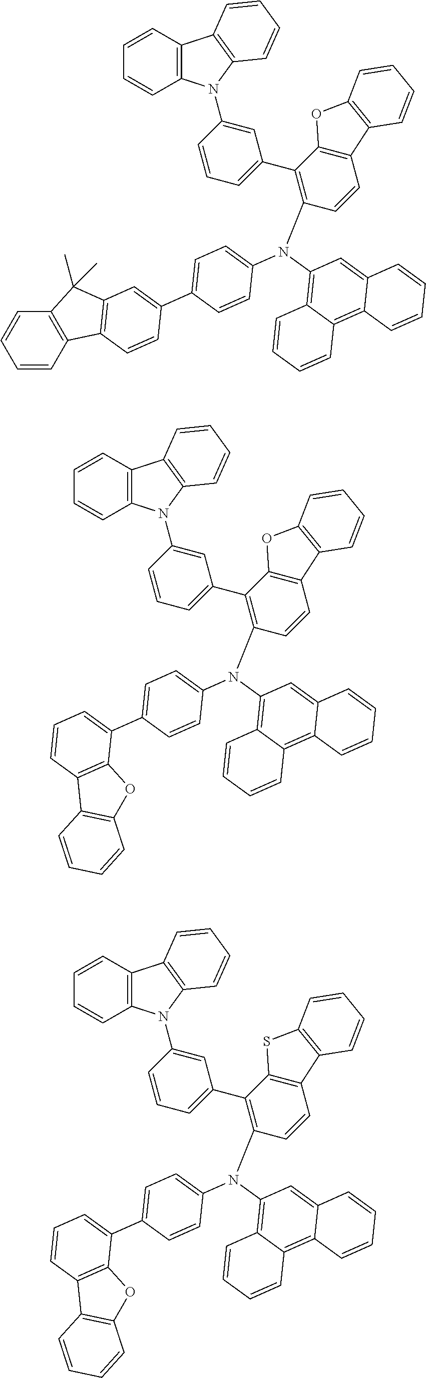 [SMILES: CC1(C)c2ccccc2-c2ccc(-c3ccc(N(c4ccc5c(oc6ccccc65)c4-c4cccc(-n5c6ccccc6c6ccccc65)c4)c4cc5ccccc5c5ccccc45)cc3)cc21 ; c1cc(-c2c(N(c3ccc(-c4cccc5c4oc4ccccc45)cc3)c3cc4ccccc4c4ccccc34)ccc3c2oc2ccccc23)cc(-n2c3ccccc3c3ccccc32)c1 ; c1cc(-c2c(N(c3ccc(-c4cccc5c4oc4ccccc45)cc3)c3cc4ccccc4c4ccccc34)ccc3c2sc2ccccc23)cc(-n2c3ccccc3c3ccccc32)c1]

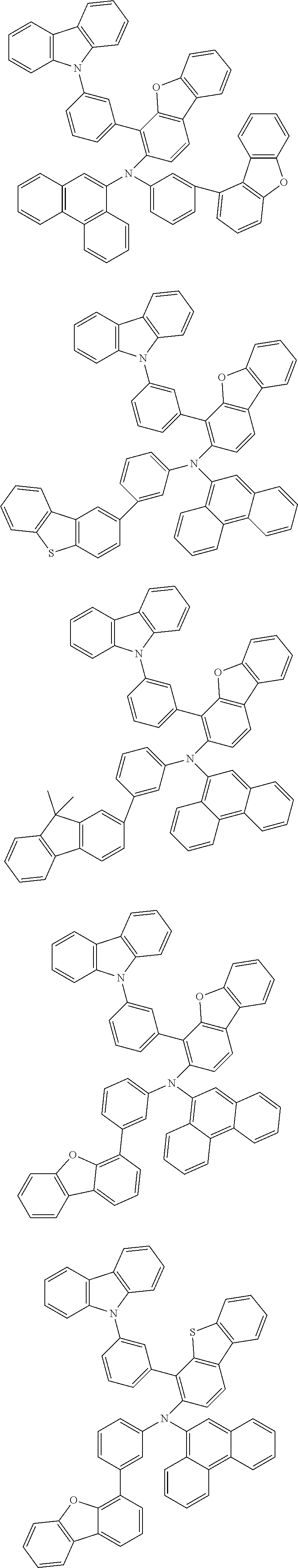

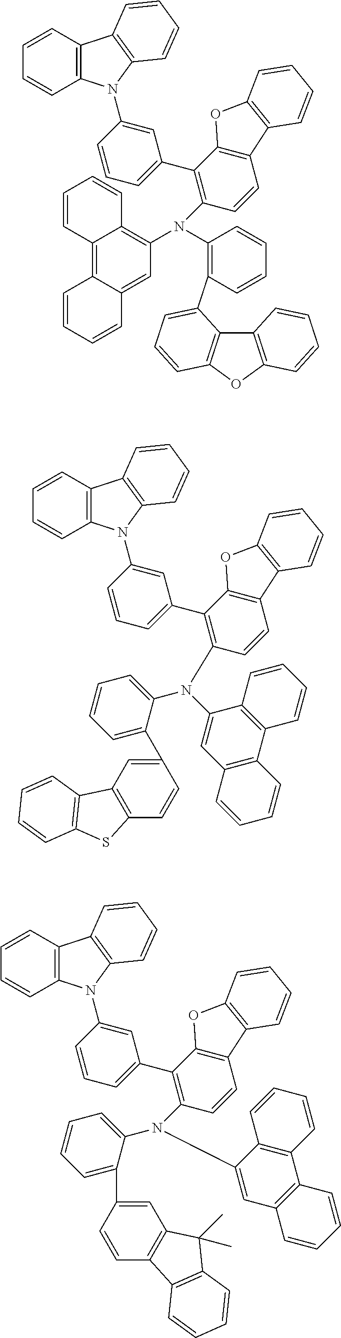 [SMILES: c1cc(-c2c(N(c3ccccc3-c3cccc4oc5ccccc5c34)c3cc4ccccc4c4ccccc34)ccc3c2oc2ccccc23)cc(-n2c3ccccc3c3ccccc32)c1 ; c1cc(-c2c(N(c3ccccc3-c3ccc4sc5ccccc5c4c3)c3cc4ccccc4c4ccccc34)ccc3c2oc2ccccc23)cc(-n2c3ccccc3c3ccccc32)c1 ; CC1(C)c2ccccc2-c2ccc(-c3ccccc3N(c3ccc4c(oc5ccccc54)c3-c3cccc(-n4c5ccccc5c5ccccc54)c3)c3cc4ccccc4c4ccccc34)cc21]

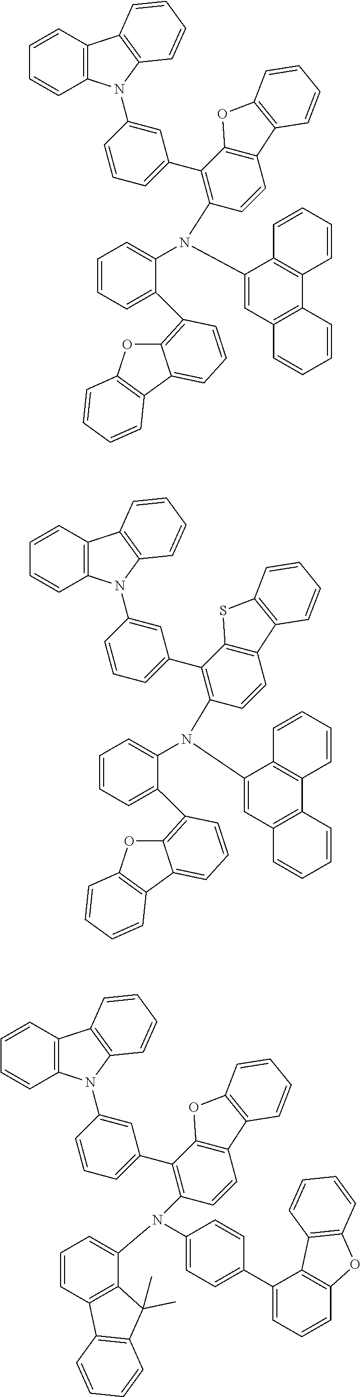 [SMILES: c1cc(-c2c(N(c3ccccc3-c3cccc4c3oc3ccccc34)c3cc4ccccc4c4ccccc34)ccc3c2oc2ccccc23)cc(-n2c3ccccc3c3ccccc32)c1 ; c1cc(-c2c(N(c3ccccc3-c3cccc4c3oc3ccccc34)c3cc4ccccc4c4ccccc34)ccc3c2sc2ccccc23)cc(-n2c3ccccc3c3ccccc32)c1 ; CC1(C)c2ccccc2-c2cccc(N(c3ccc(-c4cccc5oc6ccccc6c45)cc3)c3ccc4c(oc5ccccc54)c3-c3cccc(-n4c5ccccc5c5ccccc54)c3)c21]

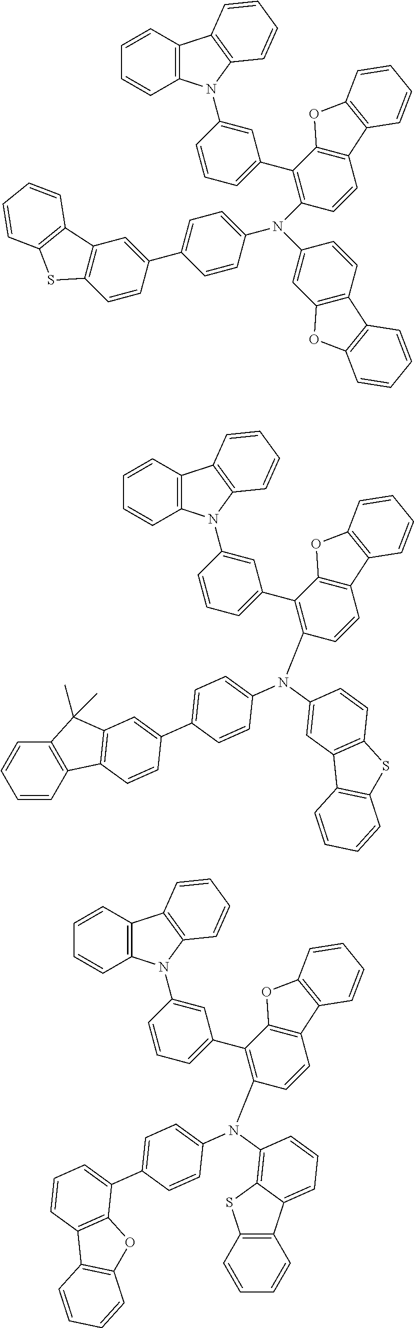 [SMILES: c1cc(-c2c(N(c3ccc(-c4ccc5sc6ccccc6c5c4)cc3)c3ccc4c(c3)oc3ccccc34)ccc3c2oc2ccccc23)cc(-n2c3ccccc3c3ccccc32)c1 ; CC1(C)c2ccccc2-c2ccc(-c3ccc(N(c4ccc5sc6ccccc6c5c4)c4ccc5c(oc6ccccc65)c4-c4cccc(-n5c6ccccc6c6ccccc65)c4)cc3)cc21 ; c1cc(-c2c(N(c3ccc(-c4cccc5c4oc4ccccc45)cc3)c3cccc4c3sc3ccccc34)ccc3c2oc2ccccc23)cc(-n2c3ccccc3c3ccccc32)c1]

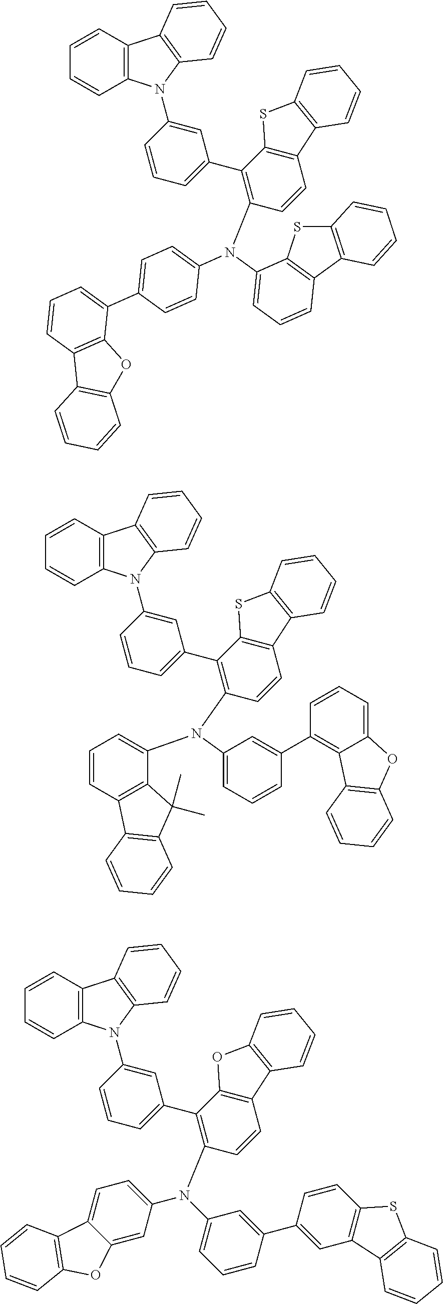 [SMILES: c1cc(-c2c(N(c3ccc(-c4cccc5c4oc4ccccc45)cc3)c3cccc4c3sc3ccccc34)ccc3c2sc2ccccc23)cc(-n2c3ccccc3c3ccccc32)c1 ; CC1(C)c2ccccc2-c2cccc(N(c3cccc(-c4cccc5oc6ccccc6c45)c3)c3ccc4c(sc5ccccc54)c3-c3cccc(-n4c5ccccc5c5ccccc54)c3)c21 ; c1cc(-c2ccc3sc4ccccc4c3c2)cc(N(c2ccc3c(c2)oc2ccccc23)c2ccc3c(oc4ccccc43)c2-c2cccc(-n3c4ccccc4c4ccccc43)c2)c1]

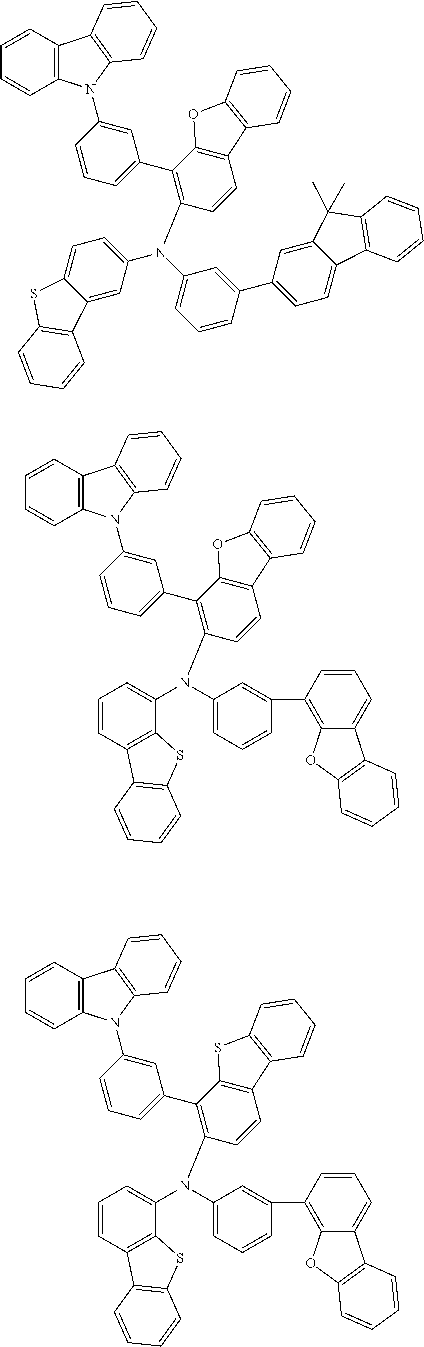 [SMILES: CC1(C)c2ccccc2-c2ccc(-c3cccc(N(c4ccc5sc6ccccc6c5c4)c4ccc5c(oc6ccccc65)c4-c4cccc(-n5c6ccccc6c6ccccc65)c4)c3)cc21 ; c1cc(-c2cccc3c2oc2ccccc23)cc(N(c2ccc3c(oc4ccccc43)c2-c2cccc(-n3c4ccccc4c4ccccc43)c2)c2cccc3c2sc2ccccc23)c1 ; c1cc(-c2cccc3c2oc2ccccc23)cc(N(c2ccc3c(sc4ccccc43)c2-c2cccc(-n3c4ccccc4c4ccccc43)c2)c2cccc3c2sc2ccccc23)c1]

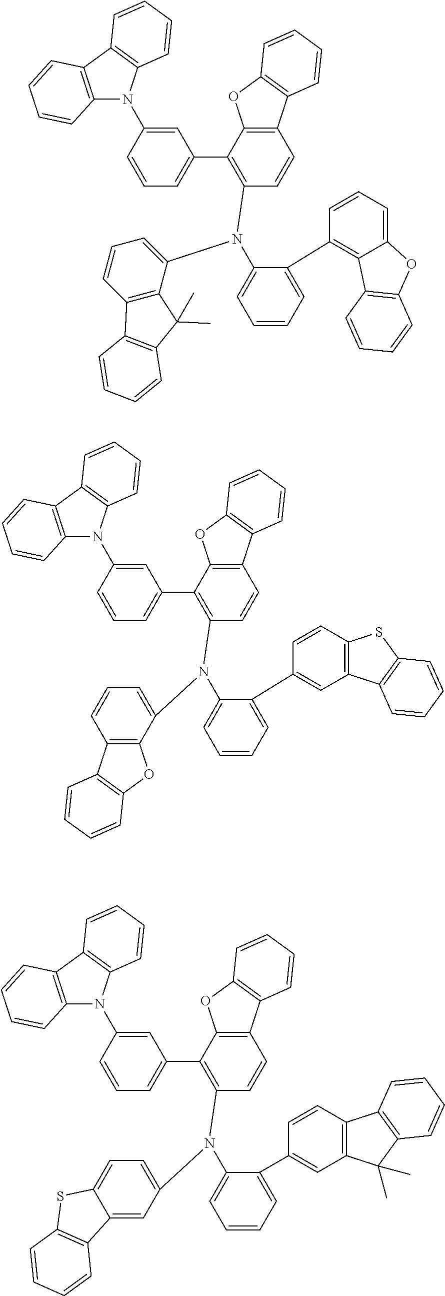

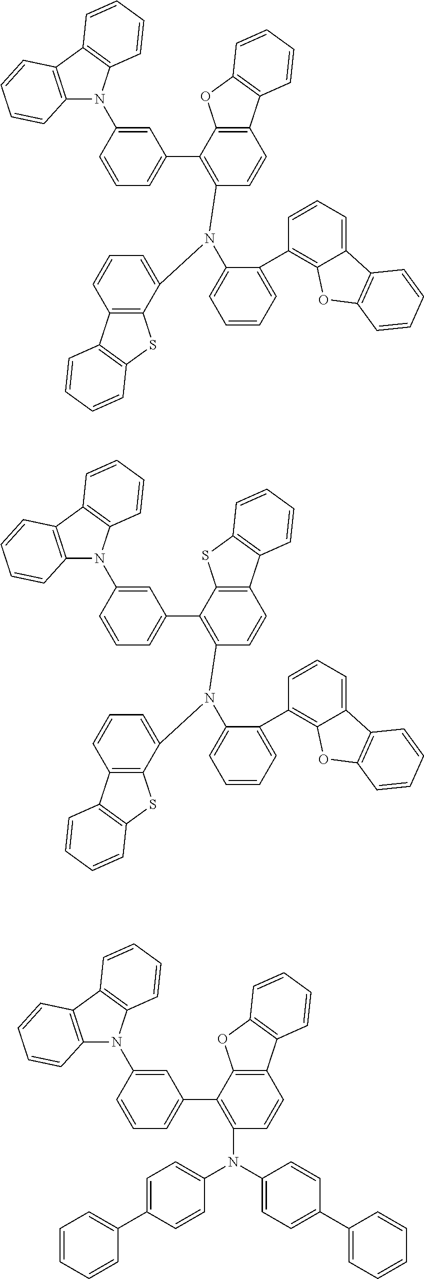 [SMILES: c1cc(-c2c(N(c3ccccc3-c3cccc4c3oc3ccccc34)c3cccc4c3sc3ccccc34)ccc3c2oc2ccccc23)cc(-n2c3ccccc3c3ccccc32)c1 ; c1cc(-c2c(N(c3ccccc3-c3cccc4c3oc3ccccc34)c3cccc4c3sc3ccccc34)ccc3c2sc2ccccc23)cc(-n2c3ccccc3c3ccccc32)c1 ; c1ccc(-c2ccc(N(c3ccc(-c4ccccc4)cc3)c3ccc4c(oc5ccccc54)c3-c3cccc(-n4c5ccccc5c5ccccc54)c3)cc2)cc1]

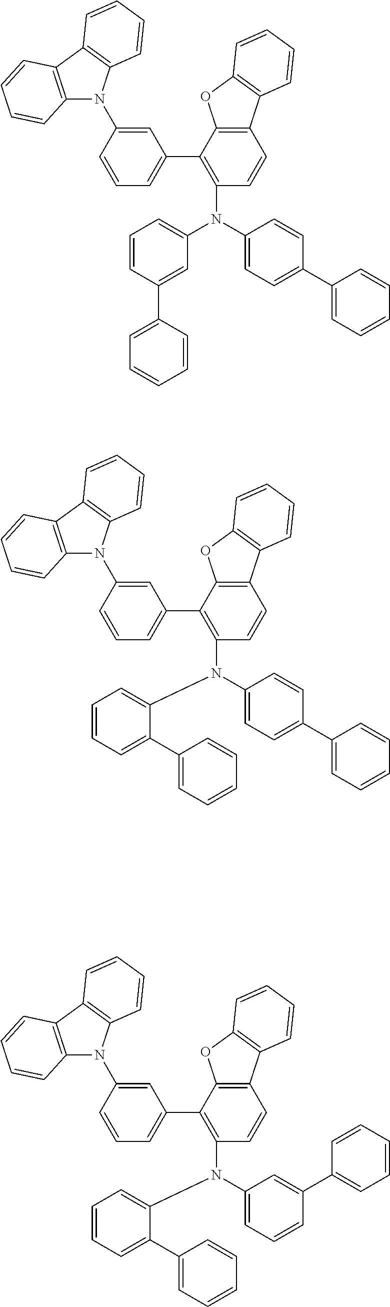 [SMILES: c1ccc(-c2ccc(N(c3cccc(-c4ccccc4)c3)c3ccc4c(oc5ccccc54)c3-c3cccc(-n4c5ccccc5c5ccccc54)c3)cc2)cc1 ; c1ccc(-c2ccc(N(c3ccccc3-c3ccccc3)c3ccc4c(oc5ccccc54)c3-c3cccc(-n4c5ccccc5c5ccccc54)c3)cc2)cc1 ; c1ccc(-c2cccc(N(c3ccccc3-c3ccccc3)c3ccc4c(oc5ccccc54)c3-c3cccc(-n4c5ccccc5c5ccccc54)c3)c2)cc1]

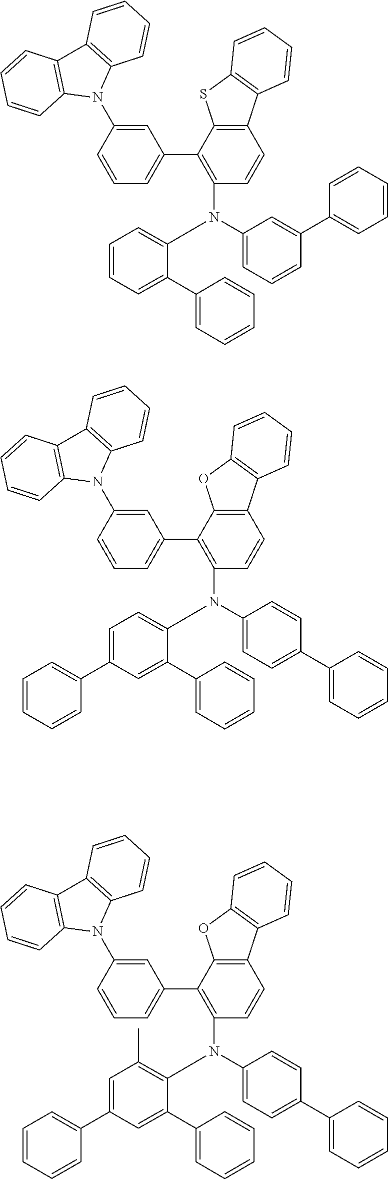 [SMILES: c1ccc(-c2cccc(N(c3ccccc3-c3ccccc3)c3ccc4c(sc5ccccc54)c3-c3cccc(-n4c5ccccc5c5ccccc54)c3)c2)cc1 ; c1ccc(-c2ccc(N(c3ccc(-c4ccccc4)cc3-c3ccccc3)c3ccc4c(oc5ccccc54)c3-c3cccc(-n4c5ccccc5c5ccccc54)c3)cc2)cc1 ; Cc1cc(-c2ccccc2)cc(-c2ccccc2)c1N(c1ccc(-c2ccccc2)cc1)c1ccc2c(oc3ccccc32)c1-c1cccc(-n2c3ccccc3c3ccccc32)c1]

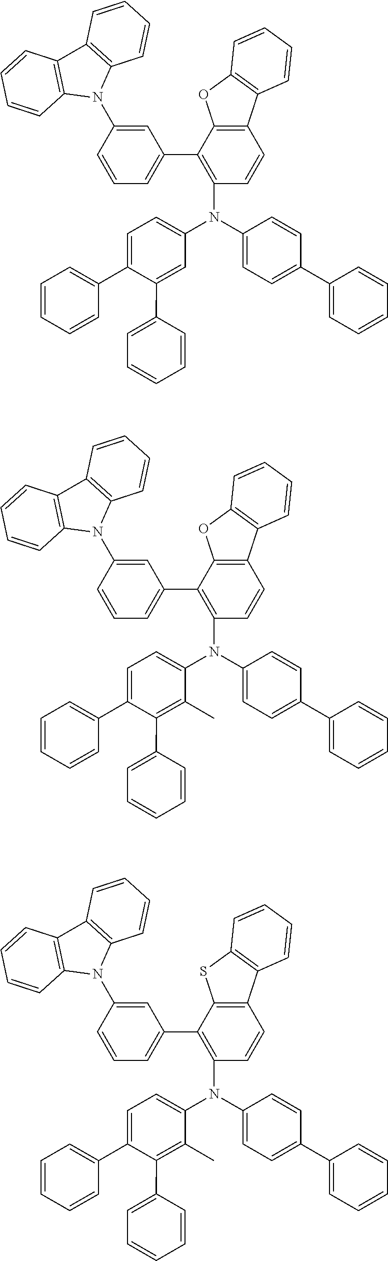 [SMILES: c1ccc(-c2ccc(N(c3ccc(-c4ccccc4)c(-c4ccccc4)c3)c3ccc4c(oc5ccccc54)c3-c3cccc(-n4c5ccccc5c5ccccc54)c3)cc2)cc1 ; Cc1c(N(c2ccc(-c3ccccc3)cc2)c2ccc3c(oc4ccccc43)c2-c2cccc(-n3c4ccccc4c4ccccc43)c2)ccc(-c2ccccc2)c1-c1ccccc1 ; Cc1c(N(c2ccc(-c3ccccc3)cc2)c2ccc3c(sc4ccccc43)c2-c2cccc(-n3c4ccccc4c4ccccc43)c2)ccc(-c2ccccc2)c1-c1ccccc1]

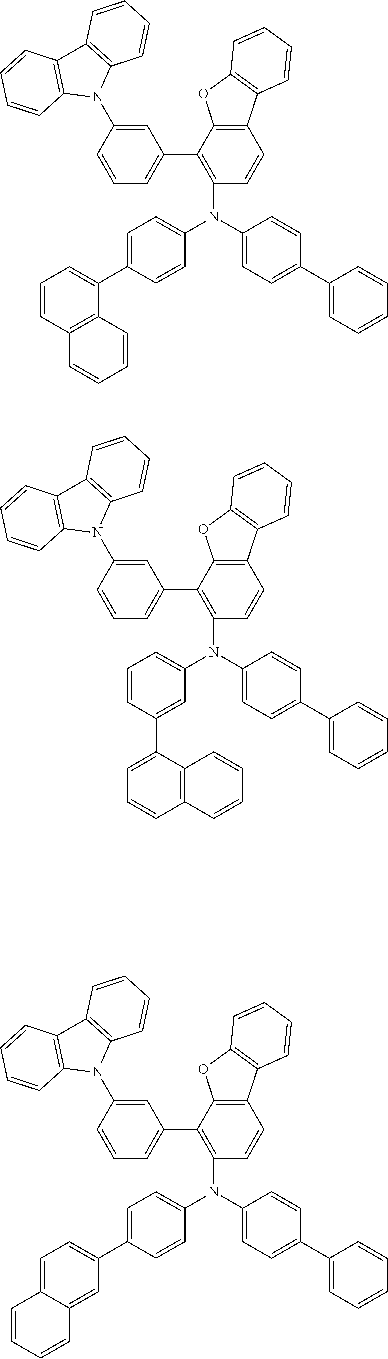 [SMILES: c1ccc(-c2ccc(N(c3ccc(-c4cccc5ccccc45)cc3)c3ccc4c(oc5ccccc54)c3-c3cccc(-n4c5ccccc5c5ccccc54)c3)cc2)cc1 ; c1ccc(-c2ccc(N(c3cccc(-c4cccc5ccccc45)c3)c3ccc4c(oc5ccccc54)c3-c3cccc(-n4c5ccccc5c5ccccc54)c3)cc2)cc1 ; c1ccc(-c2ccc(N(c3ccc(-c4ccc5ccccc5c4)cc3)c3ccc4c(oc5ccccc54)c3-c3cccc(-n4c5ccccc5c5ccccc54)c3)cc2)cc1]

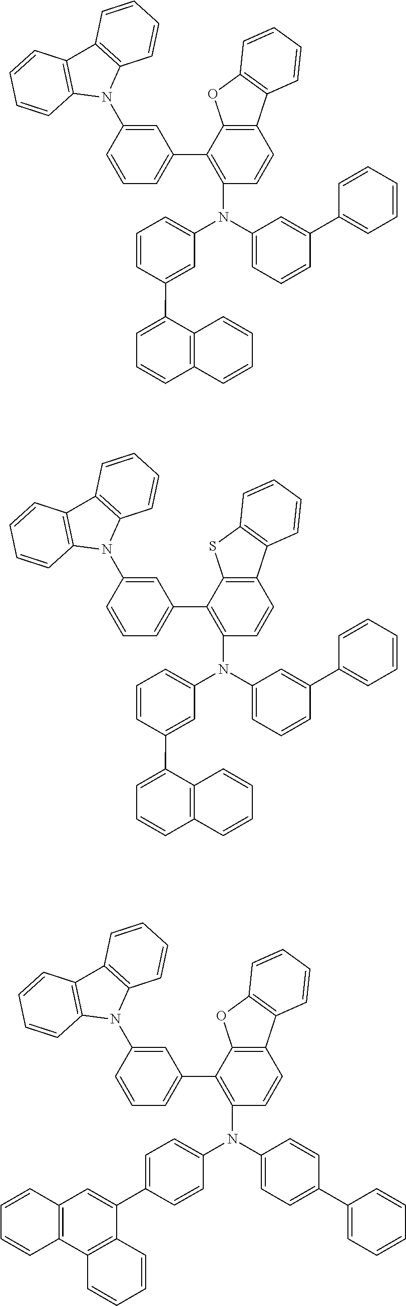 [SMILES: c1ccc(-c2cccc(N(c3cccc(-c4cccc5ccccc45)c3)c3ccc4c(oc5ccccc54)c3-c3cccc(-n4c5ccccc5c5ccccc54)c3)c2)cc1 ; c1ccc(-c2cccc(N(c3cccc(-c4cccc5ccccc45)c3)c3ccc4c(sc5ccccc54)c3-c3cccc(-n4c5ccccc5c5ccccc54)c3)c2)cc1 ; c1ccc(-c2ccc(N(c3ccc(-c4cc5ccccc5c5ccccc45)cc3)c3ccc4c(oc5ccccc54)c3-c3cccc(-n4c5ccccc5c5ccccc54)c3)cc2)cc1]

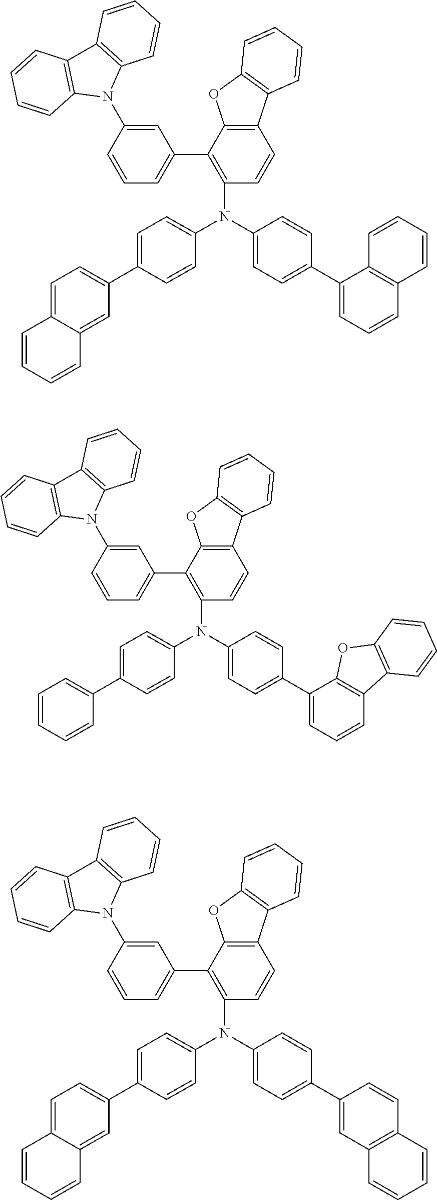 [SMILES: c1cc(-c2c(N(c3ccc(-c4ccc5ccccc5c4)cc3)c3ccc(-c4cccc5ccccc45)cc3)ccc3c2oc2ccccc23)cc(-n2c3ccccc3c3ccccc32)c1 ; c1ccc(-c2ccc(N(c3ccc(-c4cccc5c4oc4ccccc45)cc3)c3ccc4c(oc5ccccc54)c3-c3cccc(-n4c5ccccc5c5ccccc54)c3)cc2)cc1 ; c1cc(-c2c(N(c3ccc(-c4ccc5ccccc5c4)cc3)c3ccc(-c4ccc5ccccc5c4)cc3)ccc3c2oc2ccccc23)cc(-n2c3ccccc3c3ccccc32)c1]

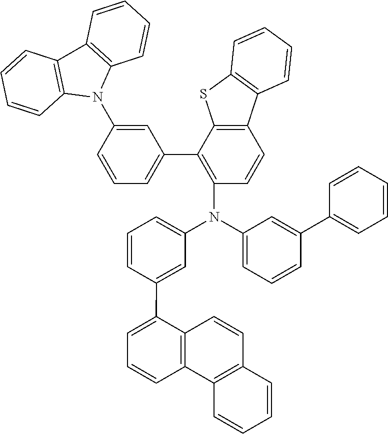 [SMILES: c1ccc(-c2cccc(N(c3cccc(-c4cccc5c4ccc4ccccc45)c3)c3ccc4c(sc5ccccc54)c3-c3cccc(-n4c5ccccc5c5ccccc54)c3)c2)cc1]

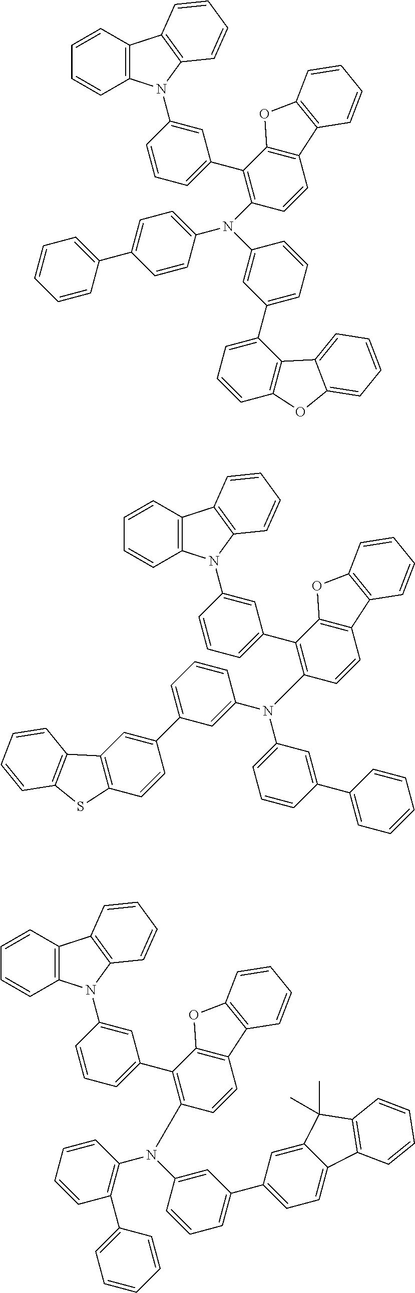 [SMILES: c1ccc(-c2ccc(N(c3cccc(-c4cccc5oc6ccccc6c45)c3)c3ccc4c(oc5ccccc54)c3-c3cccc(-n4c5ccccc5c5ccccc54)c3)cc2)cc1 ; c1ccc(-c2cccc(N(c3cccc(-c4ccc5sc6ccccc6c5c4)c3)c3ccc4c(oc5ccccc54)c3-c3cccc(-n4c5ccccc5c5ccccc54)c3)c2)cc1 ; CC1(C)c2ccccc2-c2ccc(-c3cccc(N(c4ccccc4-c4ccccc4)c4ccc5c(oc6ccccc65)c4-c4cccc(-n5c6ccccc6c6ccccc65)c4)c3)cc21]

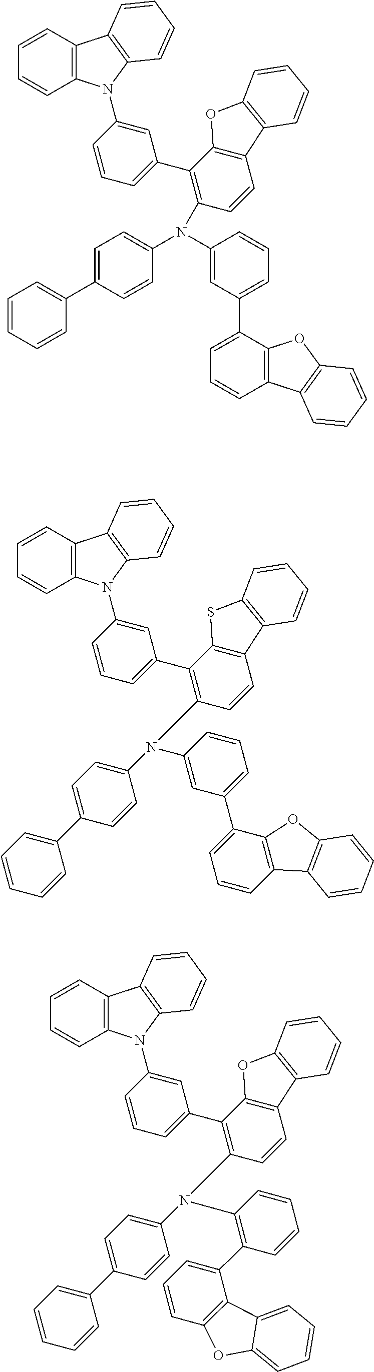 [SMILES: c1ccc(-c2ccc(N(c3cccc(-c4cccc5c4oc4ccccc45)c3)c3ccc4c(oc5ccccc54)c3-c3cccc(-n4c5ccccc5c5ccccc54)c3)cc2)cc1 ; c1ccc(-c2ccc(N(c3cccc(-c4cccc5c4oc4ccccc45)c3)c3ccc4c(sc5ccccc54)c3-c3cccc(-n4c5ccccc5c5ccccc54)c3)cc2)cc1 ; c1ccc(-c2ccc(N(c3ccccc3-c3cccc4oc5ccccc5c34)c3ccc4c(oc5ccccc54)c3-c3cccc(-n4c5ccccc5c5ccccc54)c3)cc2)cc1]

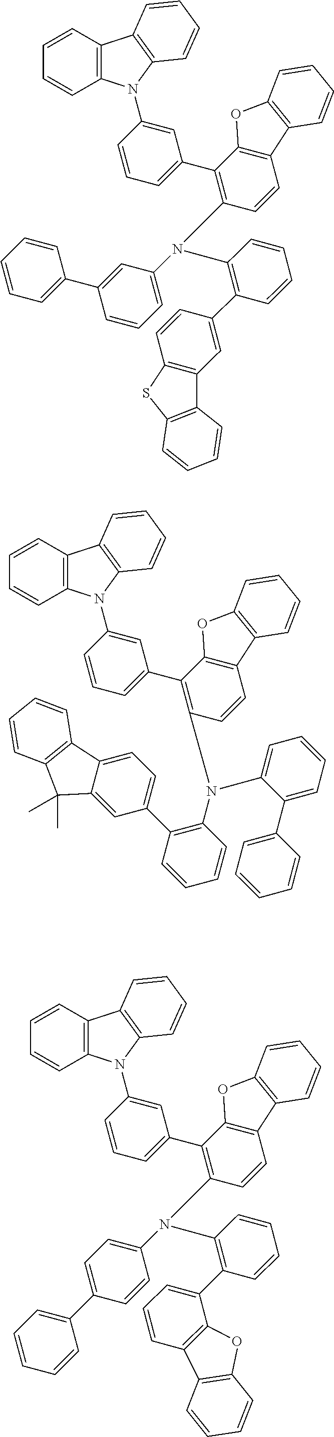 [SMILES: c1ccc(-c2cccc(N(c3ccccc3-c3ccc4sc5ccccc5c4c3)c3ccc4c(oc5ccccc54)c3-c3cccc(-n4c5ccccc5c5ccccc54)c3)c2)cc1 ; CC1(C)c2ccccc2-c2ccc(-c3ccccc3N(c3ccccc3-c3ccccc3)c3ccc4c(oc5ccccc54)c3-c3cccc(-n4c5ccccc5c5ccccc54)c3)cc21 ; c1ccc(-c2ccc(N(c3ccccc3-c3cccc4c3oc3ccccc34)c3ccc4c(oc5ccccc54)c3-c3cccc(-n4c5ccccc5c5ccccc54)c3)cc2)cc1]

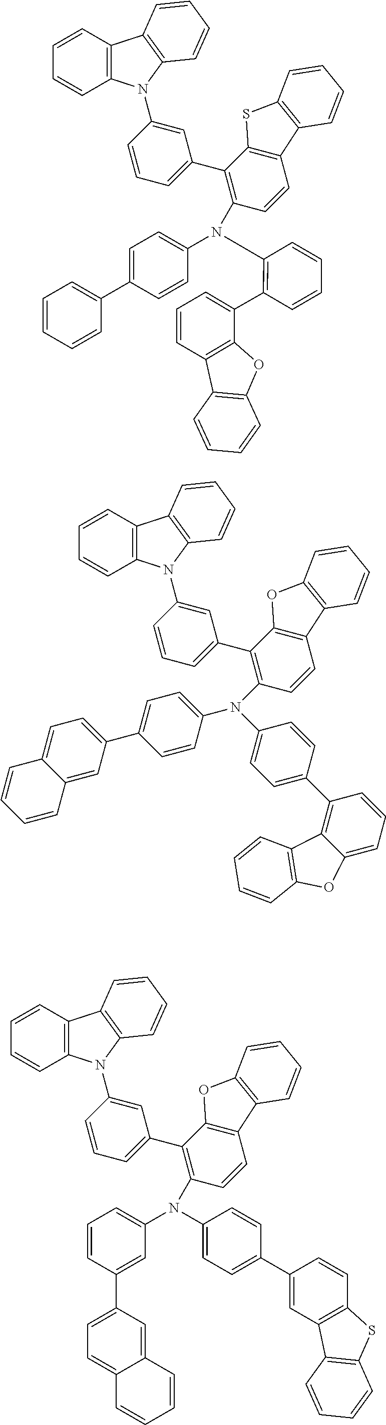

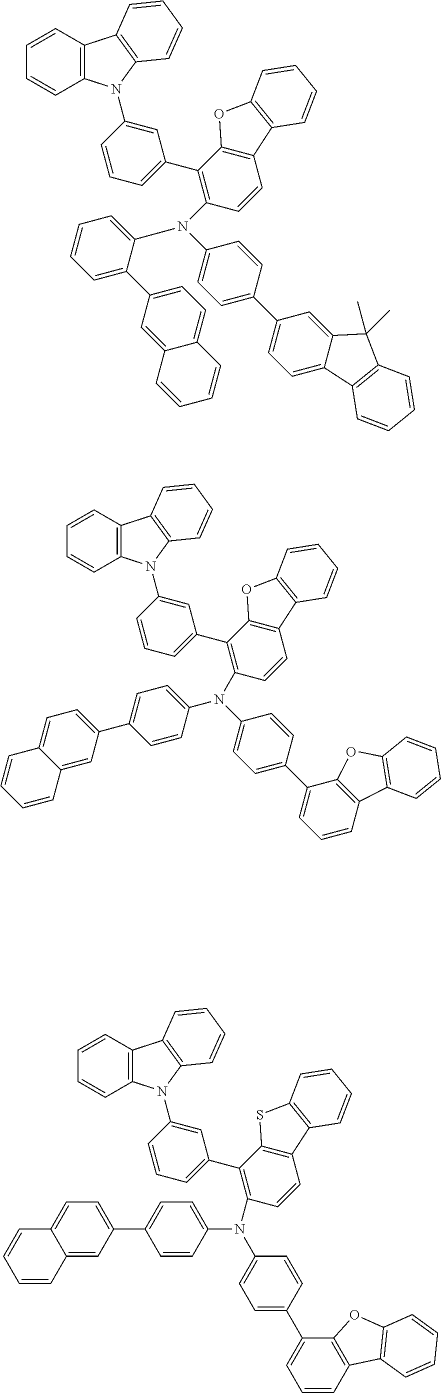 [SMILES: CC1(C)c2ccccc2-c2ccc(-c3ccc(N(c4ccccc4-c4ccc5ccccc5c4)c4ccc5c(oc6ccccc65)c4-c4cccc(-n5c6ccccc6c6ccccc65)c4)cc3)cc21 ; c1cc(-c2c(N(c3ccc(-c4ccc5ccccc5c4)cc3)c3ccc(-c4cccc5c4oc4ccccc45)cc3)ccc3c2oc2ccccc23)cc(-n2c3ccccc3c3ccccc32)c1 ; c1cc(-c2c(N(c3ccc(-c4ccc5ccccc5c4)cc3)c3ccc(-c4cccc5c4oc4ccccc45)cc3)ccc3c2sc2ccccc23)cc(-n2c3ccccc3c3ccccc32)c1]

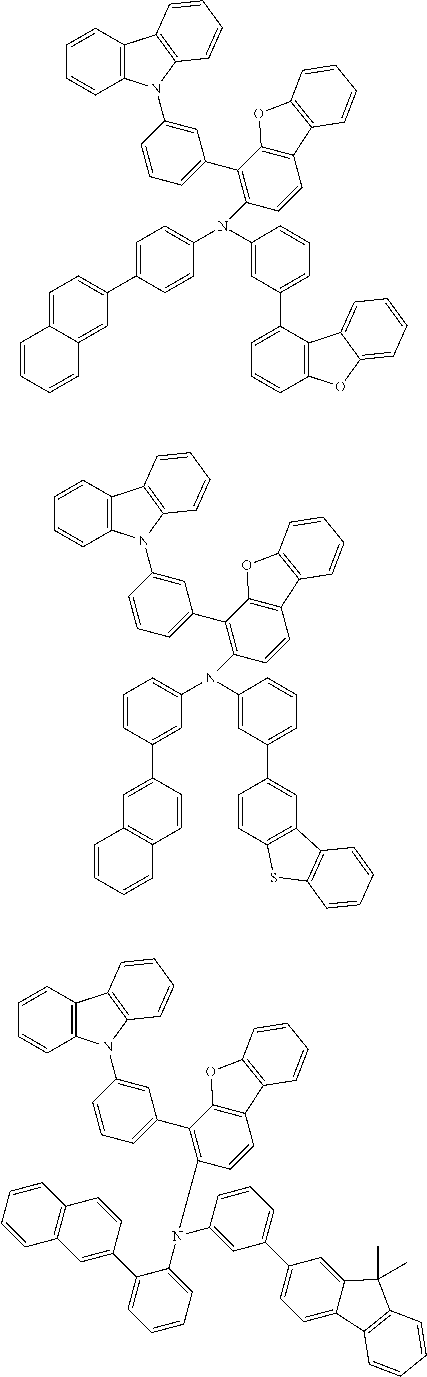 [SMILES: c1cc(-c2cccc3oc4ccccc4c23)cc(N(c2ccc(-c3ccc4ccccc4c3)cc2)c2ccc3c(oc4ccccc43)c2-c2cccc(-n3c4ccccc4c4ccccc43)c2)c1 ; c1cc(-c2ccc3ccccc3c2)cc(N(c2cccc(-c3ccc4sc5ccccc5c4c3)c2)c2ccc3c(oc4ccccc43)c2-c2cccc(-n3c4ccccc4c4ccccc43)c2)c1 ; CC1(C)c2ccccc2-c2ccc(-c3cccc(N(c4ccccc4-c4ccc5ccccc5c4)c4ccc5c(oc6ccccc65)c4-c4cccc(-n5c6ccccc6c6ccccc65)c4)c3)cc21]

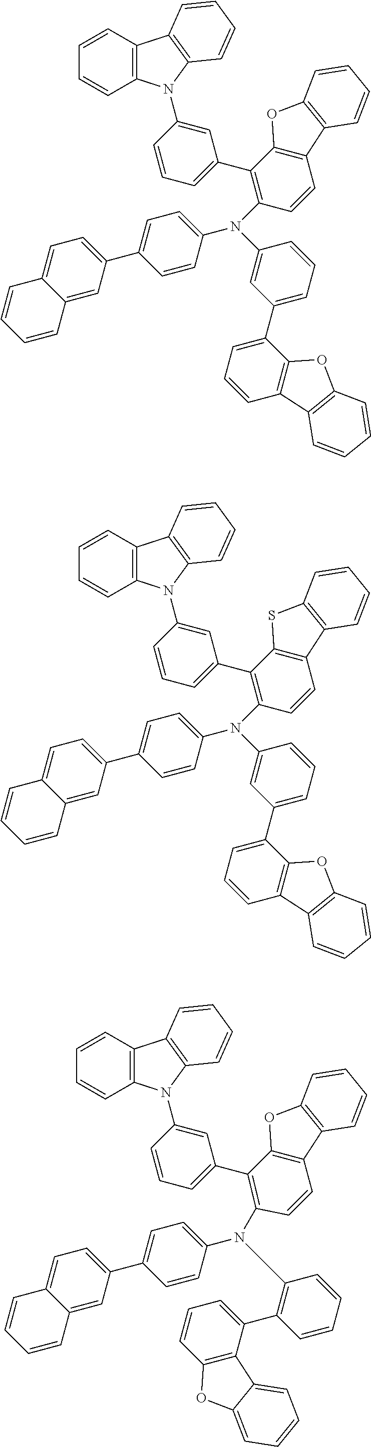 [SMILES: c1cc(-c2cccc3c2oc2ccccc23)cc(N(c2ccc(-c3ccc4ccccc4c3)cc2)c2ccc3c(oc4ccccc43)c2-c2cccc(-n3c4ccccc4c4ccccc43)c2)c1 ; c1cc(-c2cccc3c2oc2ccccc23)cc(N(c2ccc(-c3ccc4ccccc4c3)cc2)c2ccc3c(sc4ccccc43)c2-c2cccc(-n3c4ccccc4c4ccccc43)c2)c1 ; c1cc(-c2c(N(c3ccc(-c4ccc5ccccc5c4)cc3)c3ccccc3-c3cccc4oc5ccccc5c34)ccc3c2oc2ccccc23)cc(-n2c3ccccc3c3ccccc32)c1]

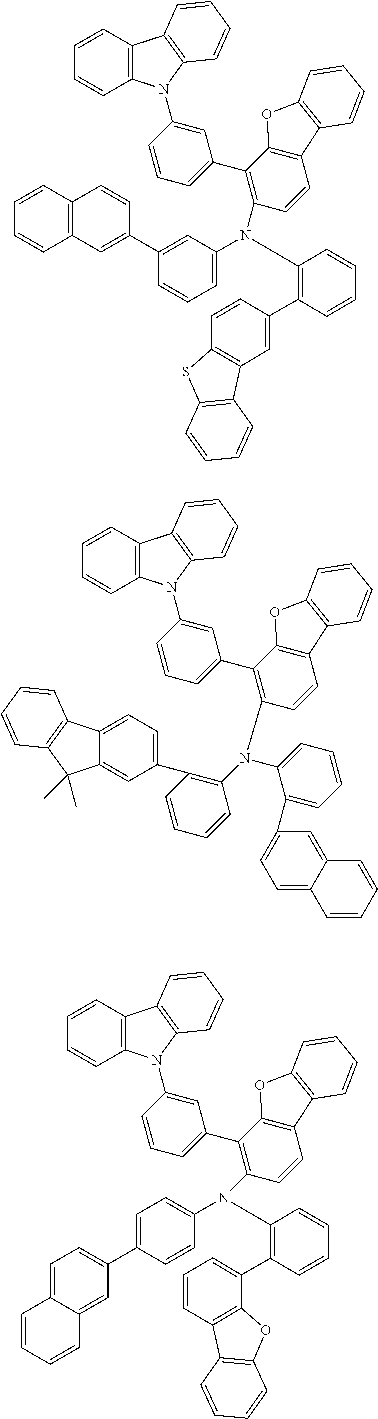

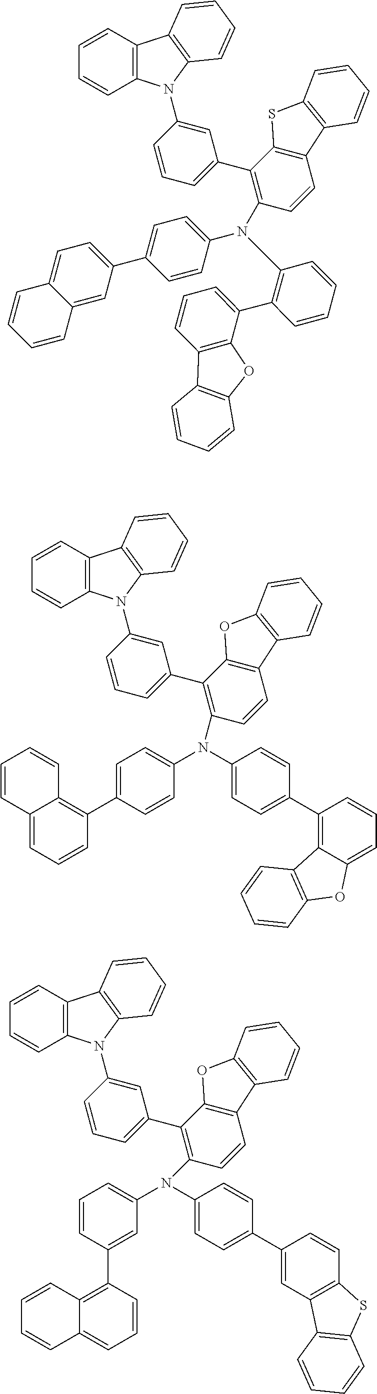 [SMILES: c1cc(-c2c(N(c3ccc(-c4ccc5ccccc5c4)cc3)c3ccccc3-c3cccc4c3oc3ccccc34)ccc3c2sc2ccccc23)cc(-n2c3ccccc3c3ccccc32)c1 ; c1cc(-c2c(N(c3ccc(-c4cccc5ccccc45)cc3)c3ccc(-c4cccc5oc6ccccc6c45)cc3)ccc3c2oc2ccccc23)cc(-n2c3ccccc3c3ccccc32)c1 ; c1cc(-c2cccc3ccccc23)cc(N(c2ccc(-c3ccc4sc5ccccc5c4c3)cc2)c2ccc3c(oc4ccccc43)c2-c2cccc(-n3c4ccccc4c4ccccc43)c2)c1]

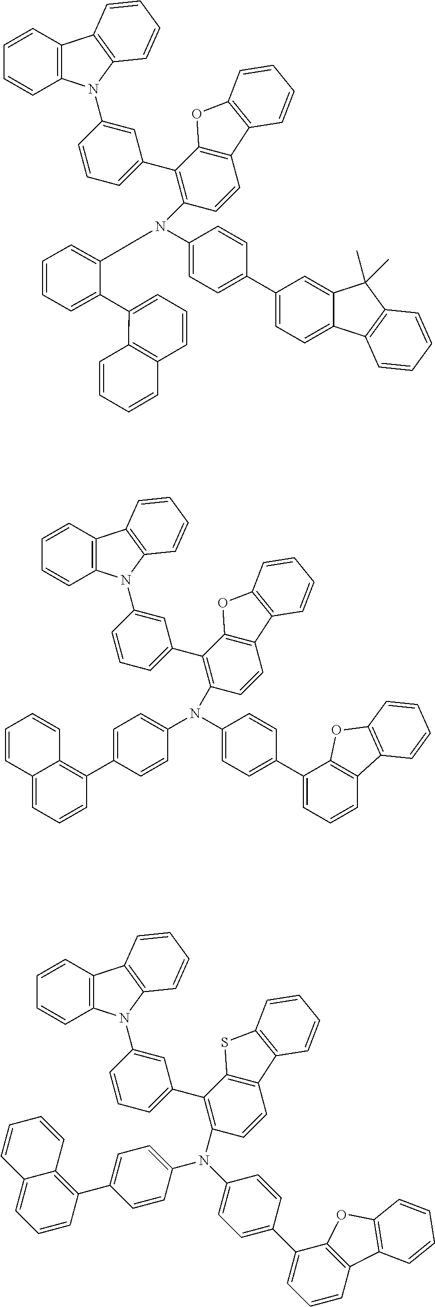 [SMILES: CC1(C)c2ccccc2-c2ccc(-c3ccc(N(c4ccccc4-c4cccc5ccccc45)c4ccc5c(oc6ccccc65)c4-c4cccc(-n5c6ccccc6c6ccccc65)c4)cc3)cc21 ; c1cc(-c2c(N(c3ccc(-c4cccc5ccccc45)cc3)c3ccc(-c4cccc5c4oc4ccccc45)cc3)ccc3c2oc2ccccc23)cc(-n2c3ccccc3c3ccccc32)c1 ; c1cc(-c2c(N(c3ccc(-c4cccc5ccccc45)cc3)c3ccc(-c4cccc5c4oc4ccccc45)cc3)ccc3c2sc2ccccc23)cc(-n2c3ccccc3c3ccccc32)c1]

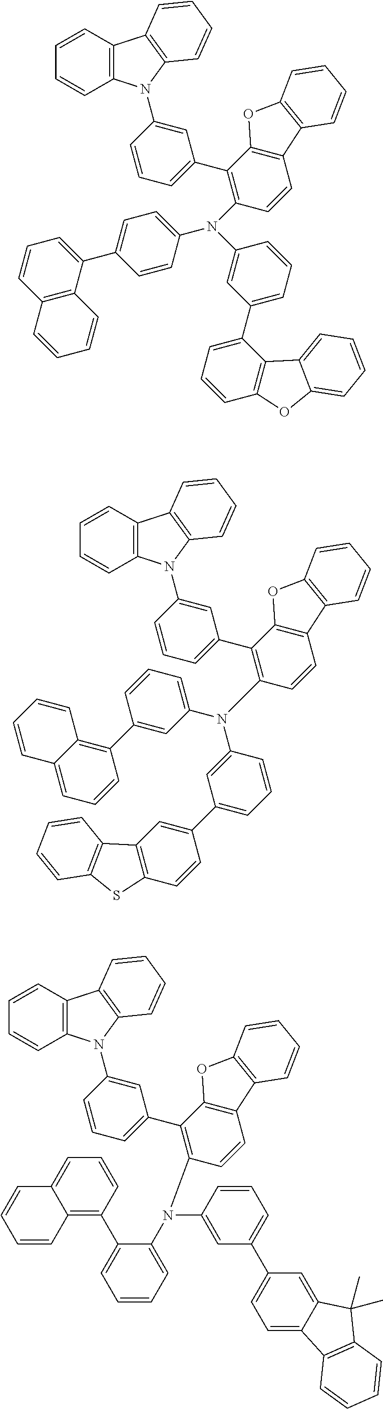 [SMILES: c1cc(-c2cccc3oc4ccccc4c23)cc(N(c2ccc(-c3cccc4ccccc34)cc2)c2ccc3c(oc4ccccc43)c2-c2cccc(-n3c4ccccc4c4ccccc43)c2)c1 ; c1cc(-c2ccc3sc4ccccc4c3c2)cc(N(c2cccc(-c3cccc4ccccc34)c2)c2ccc3c(oc4ccccc43)c2-c2cccc(-n3c4ccccc4c4ccccc43)c2)c1 ; CC1(C)c2ccccc2-c2ccc(-c3cccc(N(c4ccccc4-c4cccc5ccccc45)c4ccc5c(oc6ccccc65)c4-c4cccc(-n5c6ccccc6c6ccccc65)c4)c3)cc21]

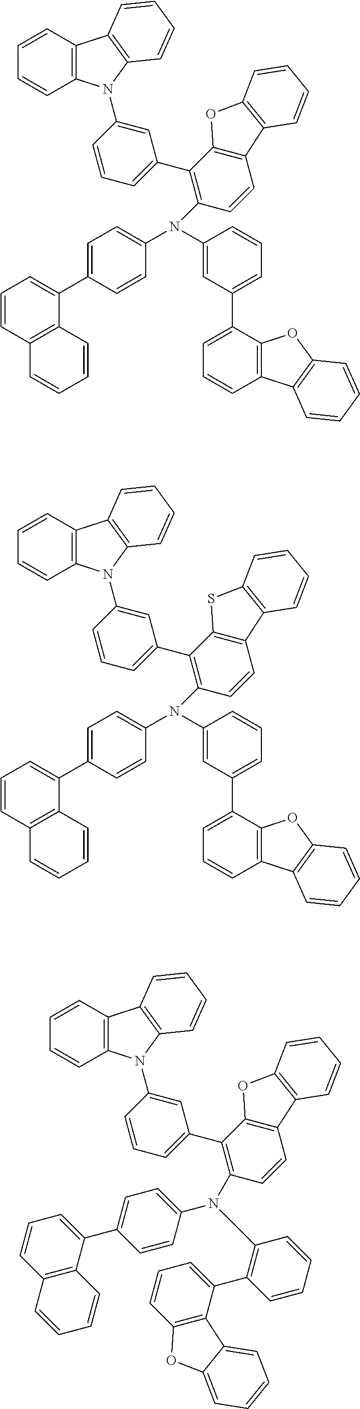 [SMILES: c1cc(-c2cccc3c2oc2ccccc23)cc(N(c2ccc(-c3cccc4ccccc34)cc2)c2ccc3c(oc4ccccc43)c2-c2cccc(-n3c4ccccc4c4ccccc43)c2)c1 ; c1cc(-c2cccc3c2oc2ccccc23)cc(N(c2ccc(-c3cccc4ccccc34)cc2)c2ccc3c(sc4ccccc43)c2-c2cccc(-n3c4ccccc4c4ccccc43)c2)c1 ; c1cc(-c2c(N(c3ccc(-c4cccc5ccccc45)cc3)c3ccccc3-c3cccc4oc5ccccc5c34)ccc3c2oc2ccccc23)cc(-n2c3ccccc3c3ccccc32)c1]

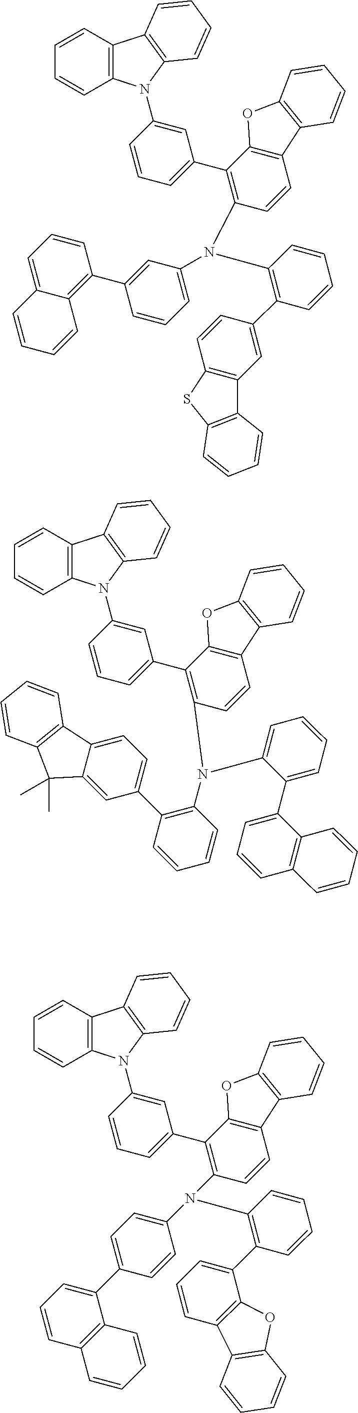 [SMILES: c1cc(-c2cccc3ccccc23)cc(N(c2ccccc2-c2ccc3sc4ccccc4c3c2)c2ccc3c(oc4ccccc43)c2-c2cccc(-n3c4ccccc4c4ccccc43)c2)c1 ; CC1(C)c2ccccc2-c2ccc(-c3ccccc3N(c3ccccc3-c3cccc4ccccc34)c3ccc4c(oc5ccccc54)c3-c3cccc(-n4c5ccccc5c5ccccc54)c3)cc21 ; c1cc(-c2c(N(c3ccc(-c4cccc5ccccc45)cc3)c3ccccc3-c3cccc4c3oc3ccccc34)ccc3c2oc2ccccc23)cc(-n2c3ccccc3c3ccccc32)c1]

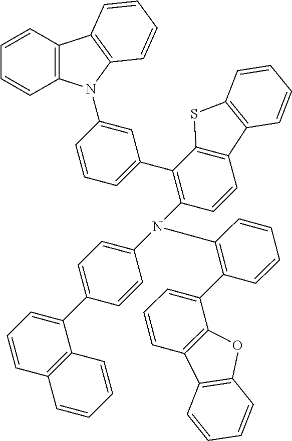 [SMILES: c1cc(-c2c(N(c3ccc(-c4cccc5ccccc45)cc3)c3ccccc3-c3cccc4c3oc3ccccc34)ccc3c2sc2ccccc23)cc(-n2c3ccccc3c3ccccc32)c1]

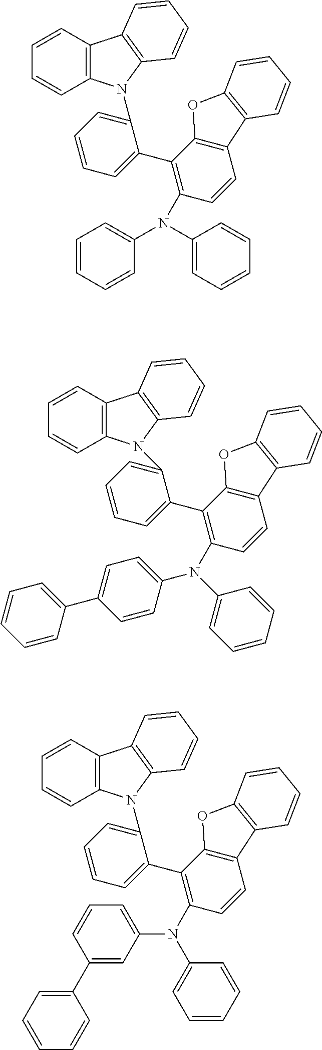 [SMILES: c1ccc(N(c2ccccc2)c2ccc3c(oc4ccccc43)c2-c2ccccc2-n2c3ccccc3c3ccccc32)cc1 ; c1ccc(-c2ccc(N(c3ccccc3)c3ccc4c(oc5ccccc54)c3-c3ccccc3-n3c4ccccc4c4ccccc43)cc2)cc1 ; c1ccc(-c2cccc(N(c3ccccc3)c3ccc4c(oc5ccccc54)c3-c3ccccc3-n3c4ccccc4c4ccccc43)c2)cc1]

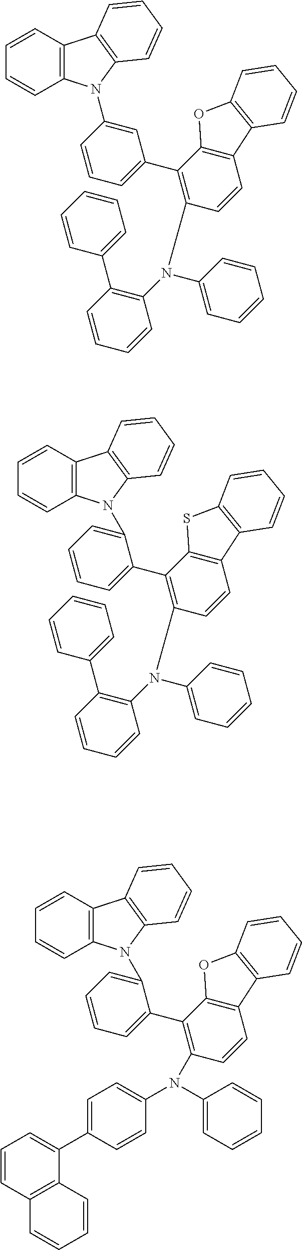 [SMILES: c1ccc(-c2ccccc2N(c2ccccc2)c2ccc3c(oc4ccccc43)c2-c2cccc(-n3c4ccccc4c4ccccc43)c2)cc1 ; c1ccc(-c2ccccc2N(c2ccccc2)c2ccc3c(sc4ccccc43)c2-c2ccccc2-n2c3ccccc3c3ccccc32)cc1 ; c1ccc(N(c2ccc(-c3cccc4ccccc34)cc2)c2ccc3c(oc4ccccc43)c2-c2ccccc2-n2c3ccccc3c3ccccc32)cc1]

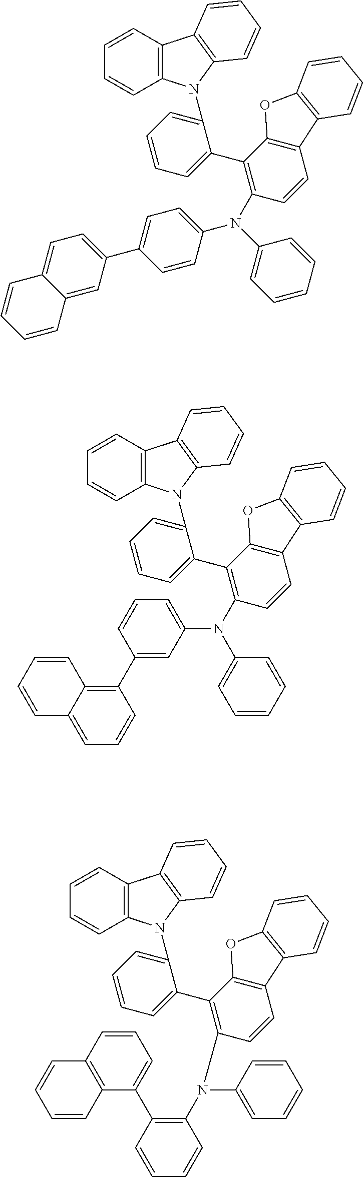 [SMILES: c1ccc(N(c2ccc(-c3ccc4ccccc4c3)cc2)c2ccc3c(oc4ccccc43)c2-c2ccccc2-n2c3ccccc3c3ccccc32)cc1 ; c1ccc(N(c2cccc(-c3cccc4ccccc34)c2)c2ccc3c(oc4ccccc43)c2-c2ccccc2-n2c3ccccc3c3ccccc32)cc1 ; c1ccc(N(c2ccccc2-c2cccc3ccccc23)c2ccc3c(oc4ccccc43)c2-c2ccccc2-n2c3ccccc3c3ccccc32)cc1]

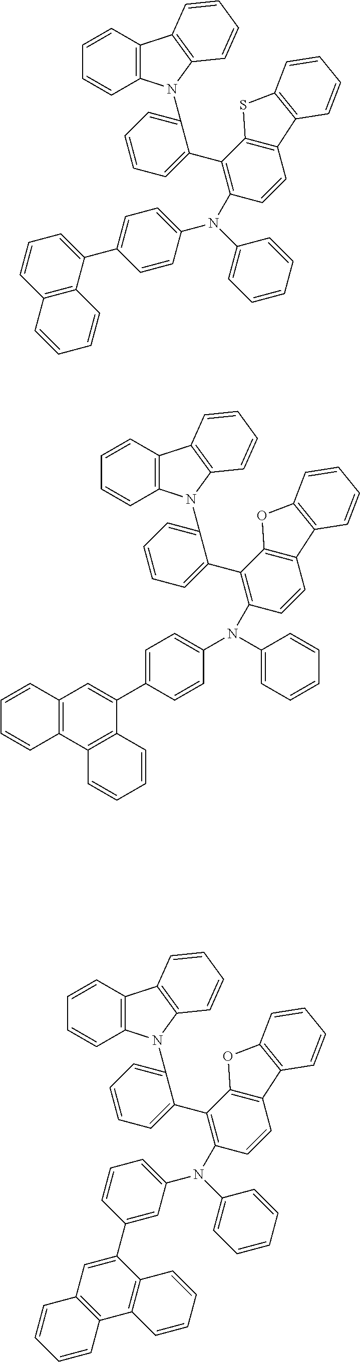 [SMILES: c1ccc(N(c2ccc(-c3cccc4ccccc34)cc2)c2ccc3c(sc4ccccc43)c2-c2ccccc2-n2c3ccccc3c3ccccc32)cc1 ; c1ccc(N(c2ccc(-c3cc4ccccc4c4ccccc34)cc2)c2ccc3c(oc4ccccc43)c2-c2ccccc2-n2c3ccccc3c3ccccc32)cc1 ; c1ccc(N(c2cccc(-c3cc4ccccc4c4ccccc34)c2)c2ccc3c(oc4ccccc43)c2-c2ccccc2-n2c3ccccc3c3ccccc32)cc1]

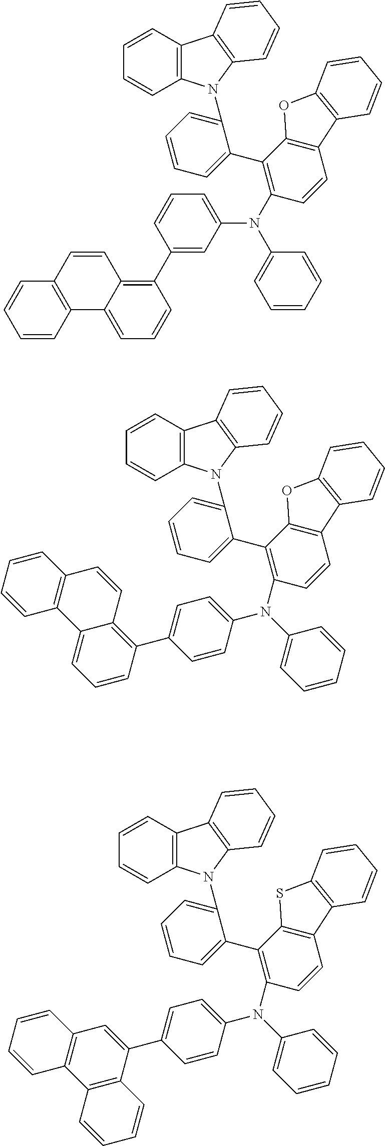 [SMILES: c1ccc(N(c2cccc(-c3cccc4c3ccc3ccccc34)c2)c2ccc3c(oc4ccccc43)c2-c2ccccc2-n2c3ccccc3c3ccccc32)cc1 ; c1ccc(N(c2ccc(-c3cccc4c3ccc3ccccc34)cc2)c2ccc3c(oc4ccccc43)c2-c2ccccc2-n2c3ccccc3c3ccccc32)cc1 ; c1ccc(N(c2ccc(-c3cc4ccccc4c4ccccc34)cc2)c2ccc3c(sc4ccccc43)c2-c2ccccc2-n2c3ccccc3c3ccccc32)cc1]

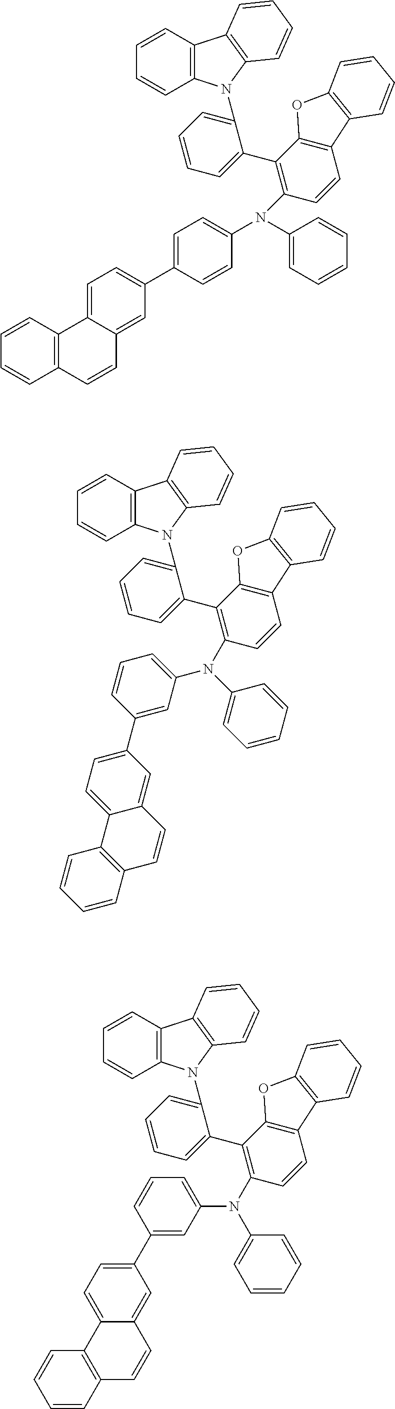 [SMILES: c1ccc(N(c2ccc(-c3ccc4c(ccc5ccccc54)c3)cc2)c2ccc3c(oc4ccccc43)c2-c2ccccc2-n2c3ccccc3c3ccccc32)cc1 ; c1ccc(N(c2cccc(-c3ccc4c(ccc5ccccc54)c3)c2)c2ccc3c(oc4ccccc43)c2-c2ccccc2-n2c3ccccc3c3ccccc32)cc1 ; c1ccc(N(c2cccc(-c3ccc4c(ccc5ccccc54)c3)c2)c2ccc3c(oc4ccccc43)c2-c2ccccc2-n2c3ccccc3c3ccccc32)cc1]

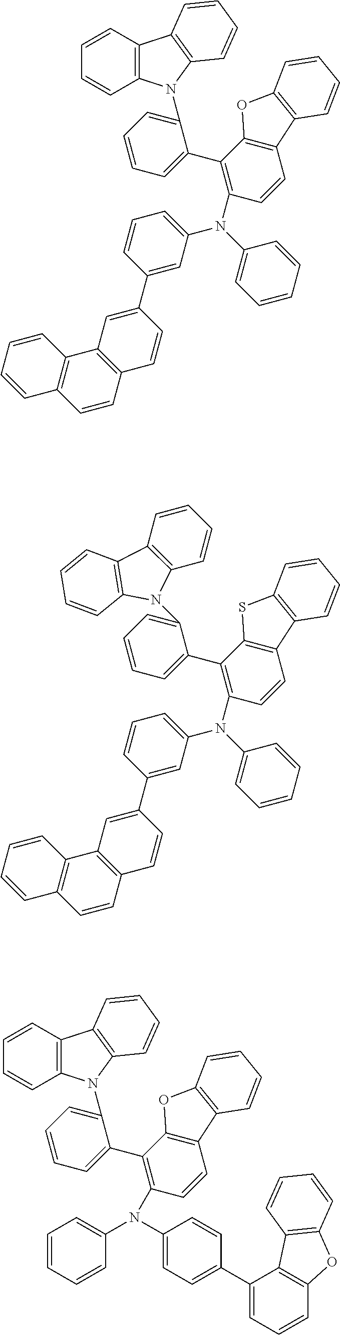 [SMILES: c1ccc(N(c2cccc(-c3ccc4ccc5ccccc5c4c3)c2)c2ccc3c(oc4ccccc43)c2-c2ccccc2-n2c3ccccc3c3ccccc32)cc1 ; c1ccc(N(c2cccc(-c3ccc4ccc5ccccc5c4c3)c2)c2ccc3c(sc4ccccc43)c2-c2ccccc2-n2c3ccccc3c3ccccc32)cc1 ; c1ccc(N(c2ccc(-c3cccc4oc5ccccc5c34)cc2)c2ccc3c(oc4ccccc43)c2-c2ccccc2-n2c3ccccc3c3ccccc32)cc1]

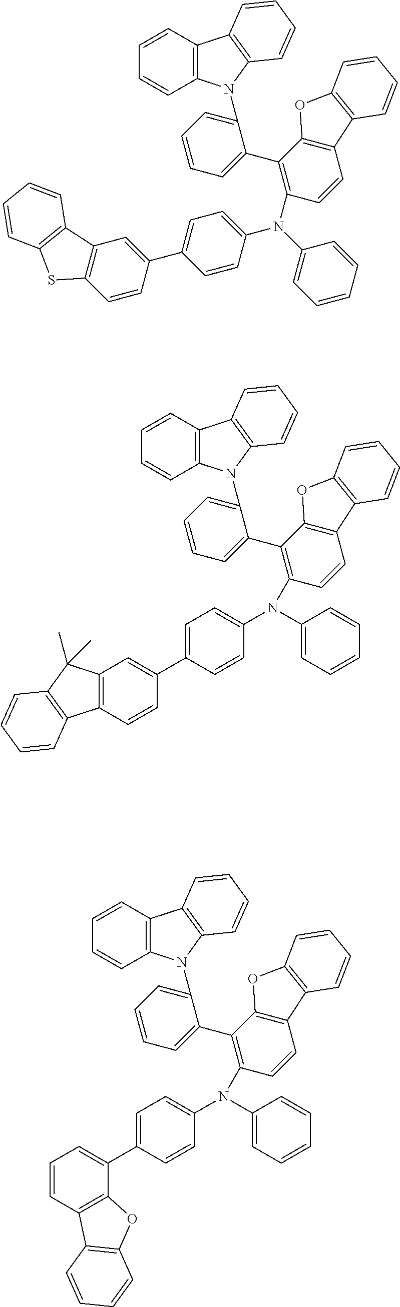 [SMILES: c1ccc(N(c2ccc(-c3ccc4sc5ccccc5c4c3)cc2)c2ccc3c(oc4ccccc43)c2-c2ccccc2-n2c3ccccc3c3ccccc32)cc1 ; CC1(C)c2ccccc2-c2ccc(-c3ccc(N(c4ccccc4)c4ccc5c(oc6ccccc65)c4-c4ccccc4-n4c5ccccc5c5ccccc54)cc3)cc21 ; c1ccc(N(c2ccc(-c3cccc4c3oc3ccccc34)cc2)c2ccc3c(oc4ccccc43)c2-c2ccccc2-n2c3ccccc3c3ccccc32)cc1]

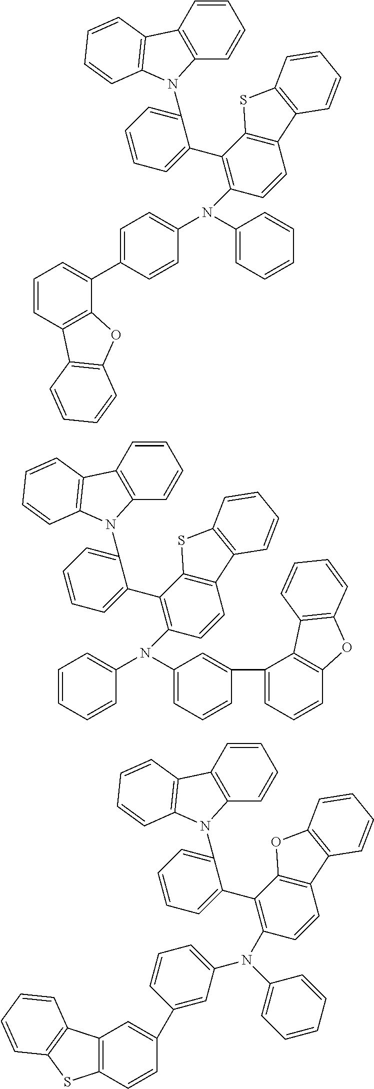

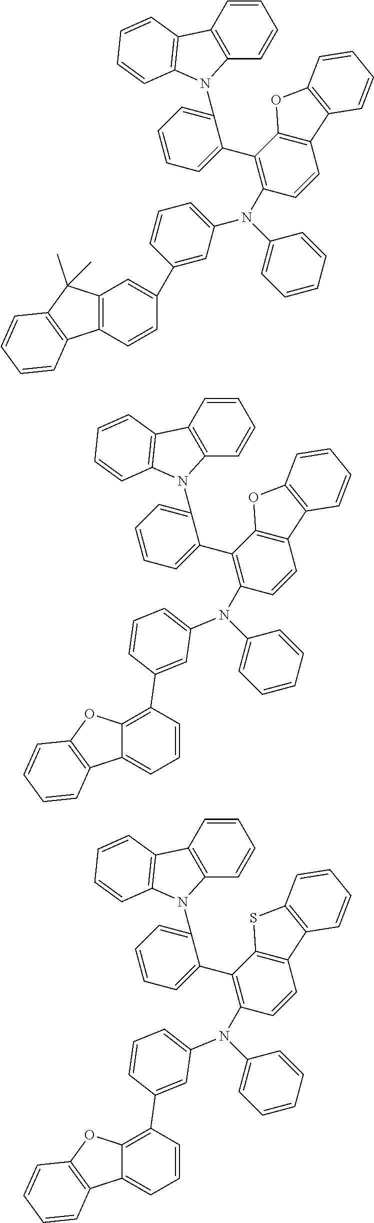 [SMILES: CC1(C)c2ccccc2-c2ccc(-c3cccc(N(c4ccccc4)c4ccc5c(oc6ccccc65)c4-c4ccccc4-n4c5ccccc5c5ccccc54)c3)cc21 ; c1ccc(N(c2cccc(-c3cccc4c3oc3ccccc34)c2)c2ccc3c(oc4ccccc43)c2-c2ccccc2-n2c3ccccc3c3ccccc32)cc1 ; c1ccc(N(c2cccc(-c3cccc4c3oc3ccccc34)c2)c2ccc3c(sc4ccccc43)c2-c2ccccc2-n2c3ccccc3c3ccccc32)cc1]

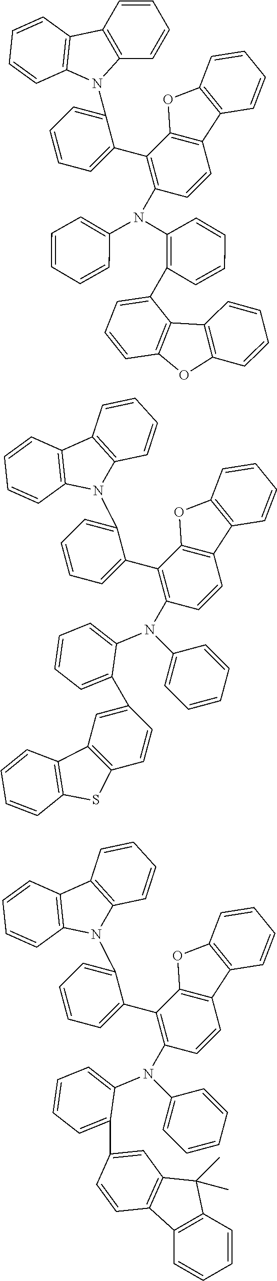

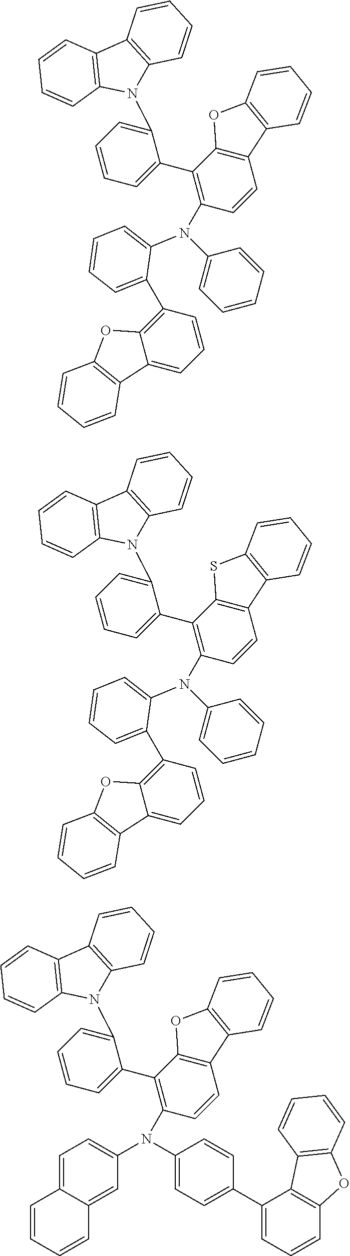

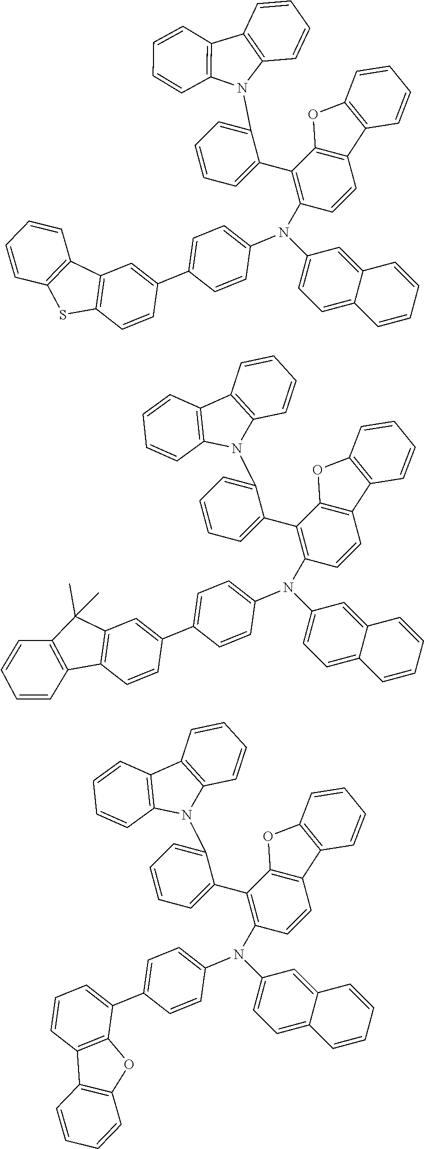 [SMILES: c1ccc(-n2c3ccccc3c3ccccc32)c(-c2c(N(c3ccc(-c4ccc5sc6ccccc6c5c4)cc3)c3ccc4ccccc4c3)ccc3c2oc2ccccc23)c1 ; CC1(C)c2ccccc2-c2ccc(-c3ccc(N(c4ccc5ccccc5c4)c4ccc5c(oc6ccccc65)c4-c4ccccc4-n4c5ccccc5c5ccccc54)cc3)cc21 ; c1ccc(-n2c3ccccc3c3ccccc32)c(-c2c(N(c3ccc(-c4cccc5c4oc4ccccc45)cc3)c3ccc4ccccc4c3)ccc3c2oc2ccccc23)c1]

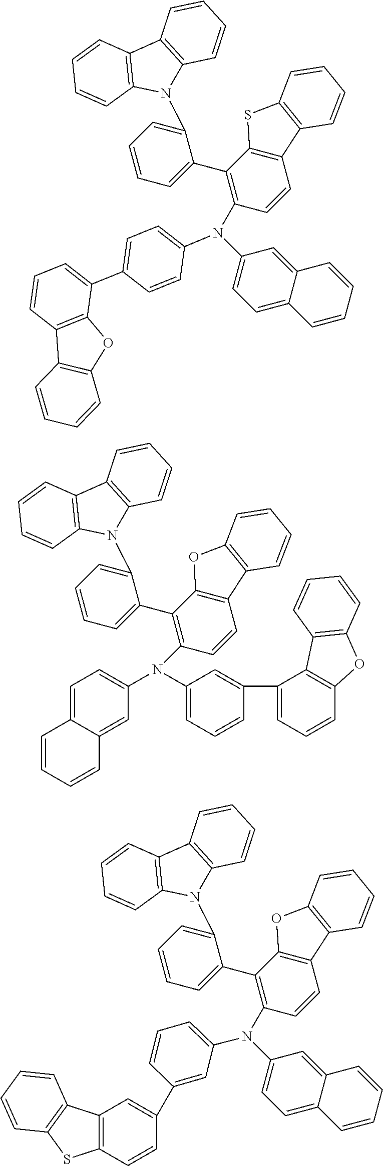

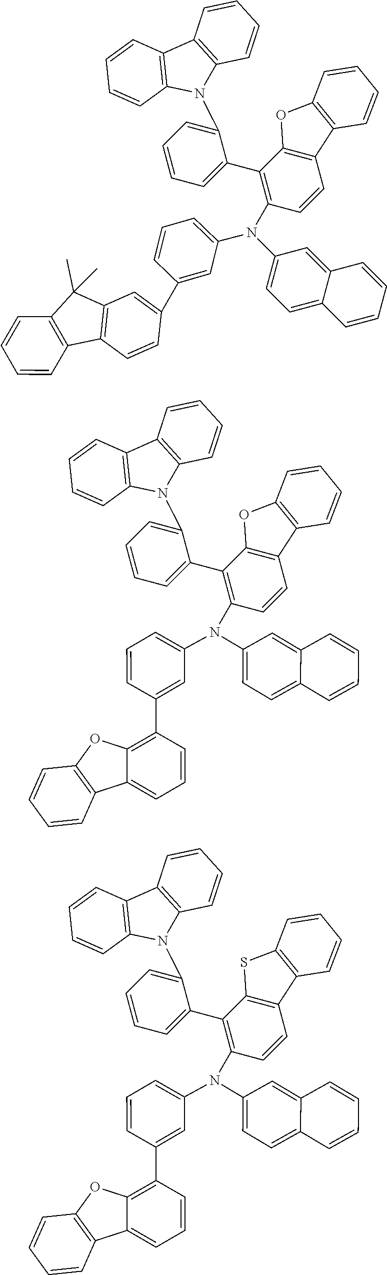 [SMILES: CC1(C)c2ccccc2-c2ccc(-c3cccc(N(c4ccc5ccccc5c4)c4ccc5c(oc6ccccc65)c4-c4ccccc4-n4c5ccccc5c5ccccc54)c3)cc21 ; c1cc(-c2cccc3c2oc2ccccc23)cc(N(c2ccc3ccccc3c2)c2ccc3c(oc4ccccc43)c2-c2ccccc2-n2c3ccccc3c3ccccc32)c1 ; c1cc(-c2cccc3c2oc2ccccc23)cc(N(c2ccc3ccccc3c2)c2ccc3c(sc4ccccc43)c2-c2ccccc2-n2c3ccccc3c3ccccc32)c1]

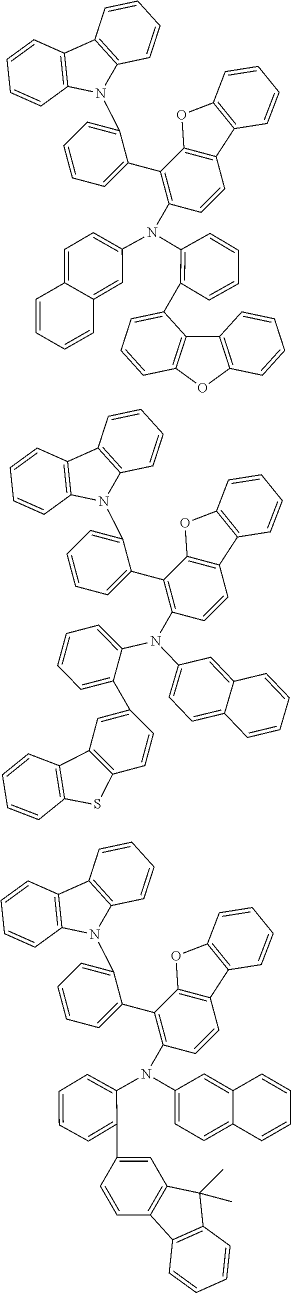

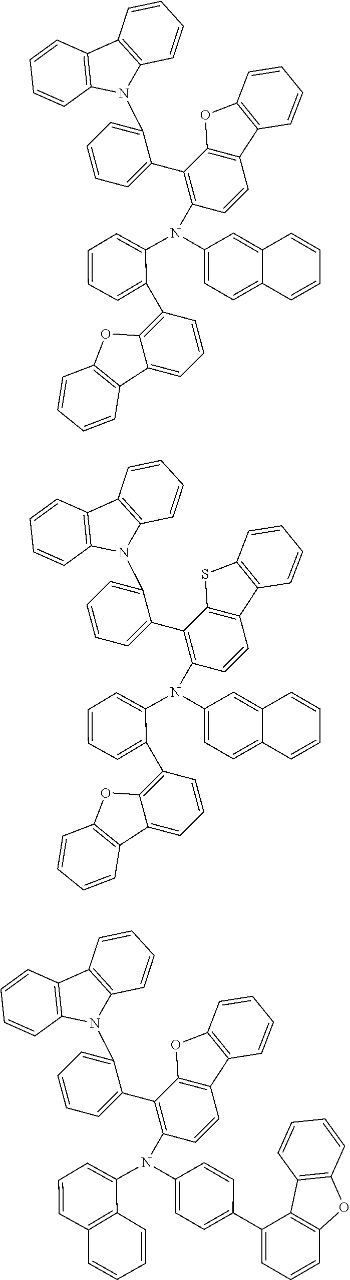 [SMILES: c1ccc(N(c2ccc3ccccc3c2)c2ccc3c(oc4ccccc43)c2-c2ccccc2-n2c3ccccc3c3ccccc32)c(-c2cccc3c2oc2ccccc23)c1 ; c1ccc(N(c2ccc3ccccc3c2)c2ccc3c(sc4ccccc43)c2-c2ccccc2-n2c3ccccc3c3ccccc32)c(-c2cccc3c2oc2ccccc23)c1 ; c1ccc(-n2c3ccccc3c3ccccc32)c(-c2c(N(c3ccc(-c4cccc5oc6ccccc6c45)cc3)c3cccc4ccccc34)ccc3c2oc2ccccc23)c1]

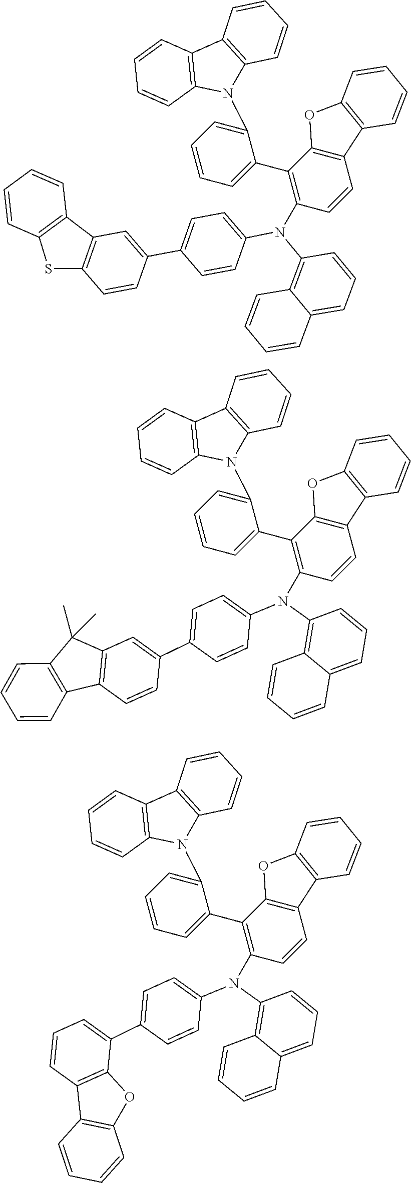

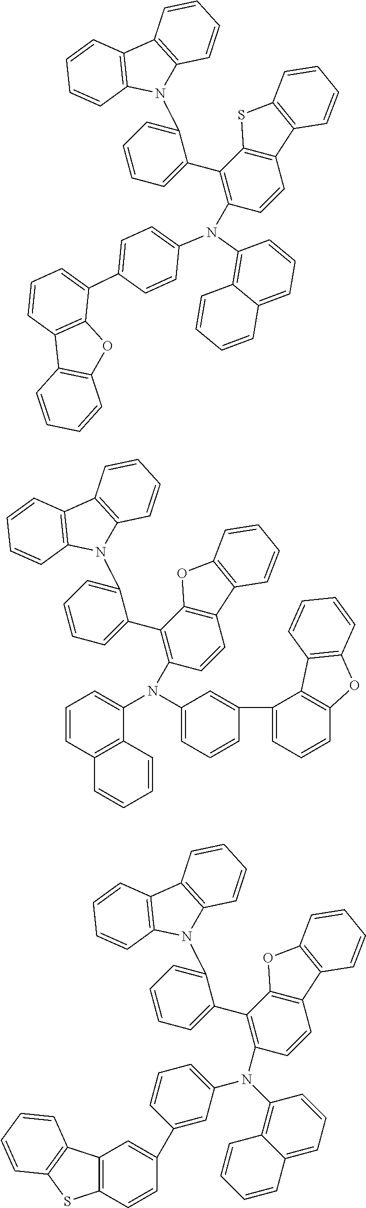

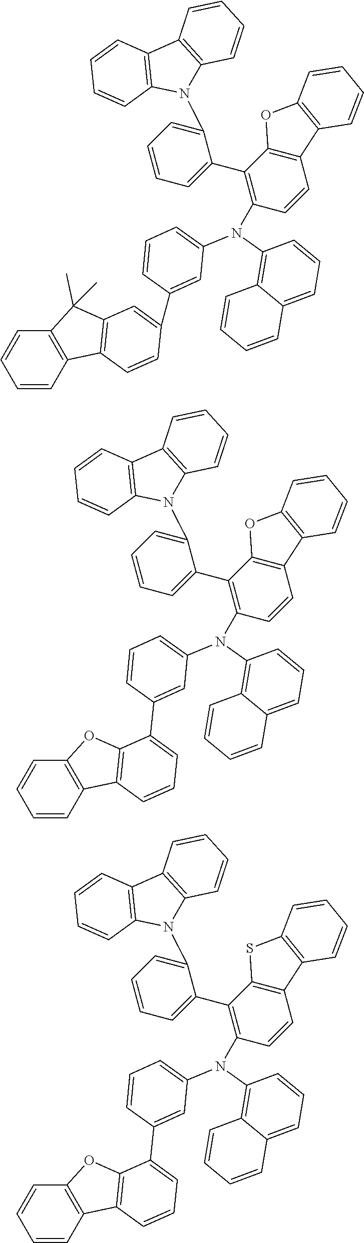 [SMILES: CC1(C)c2ccccc2-c2ccc(-c3cccc(N(c4ccc5c(oc6ccccc65)c4-c4ccccc4-n4c5ccccc5c5ccccc54)c4cccc5ccccc45)c3)cc21 ; c1cc(-c2cccc3c2oc2ccccc23)cc(N(c2ccc3c(oc4ccccc43)c2-c2ccccc2-n2c3ccccc3c3ccccc32)c2cccc3ccccc23)c1 ; c1cc(-c2cccc3c2oc2ccccc23)cc(N(c2ccc3c(sc4ccccc43)c2-c2ccccc2-n2c3ccccc3c3ccccc32)c2cccc3ccccc23)c1]

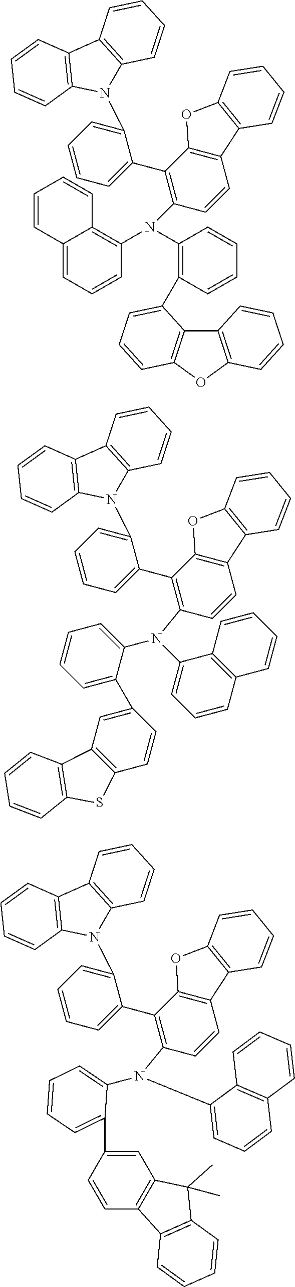

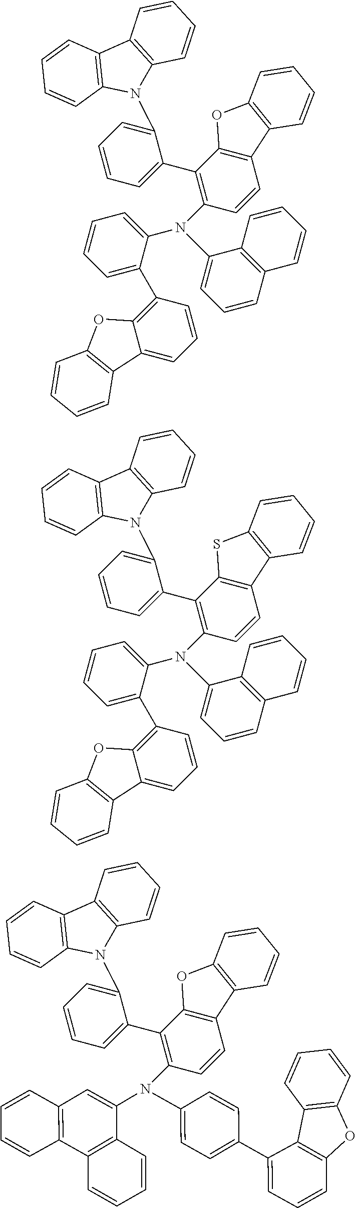

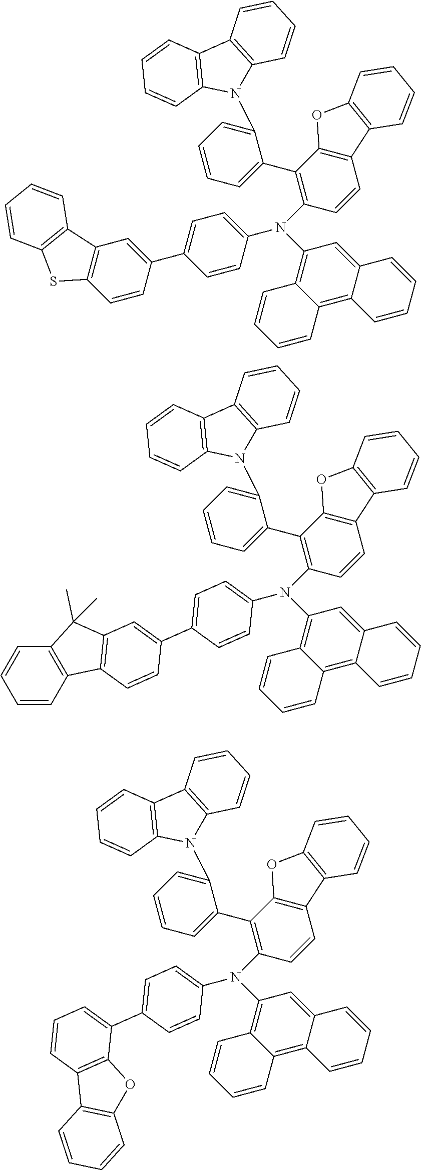

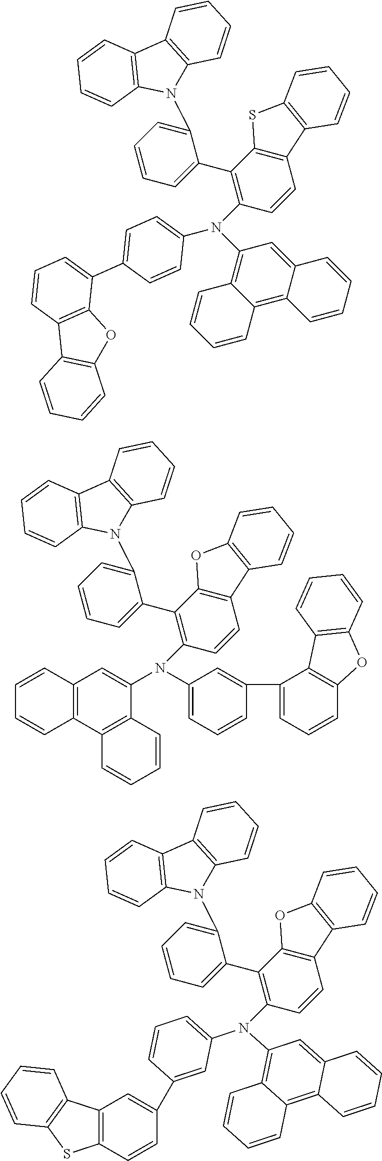 [SMILES: c1ccc(-n2c3ccccc3c3ccccc32)c(-c2c(N(c3ccc(-c4cccc5c4oc4ccccc45)cc3)c3cc4ccccc4c4ccccc34)ccc3c2sc2ccccc23)c1 ; c1cc(-c2cccc3oc4ccccc4c23)cc(N(c2ccc3c(oc4ccccc43)c2-c2ccccc2-n2c3ccccc3c3ccccc32)c2cc3ccccc3c3ccccc23)c1 ; c1cc(-c2ccc3sc4ccccc4c3c2)cc(N(c2ccc3c(oc4ccccc43)c2-c2ccccc2-n2c3ccccc3c3ccccc32)c2cc3ccccc3c3ccccc23)c1]

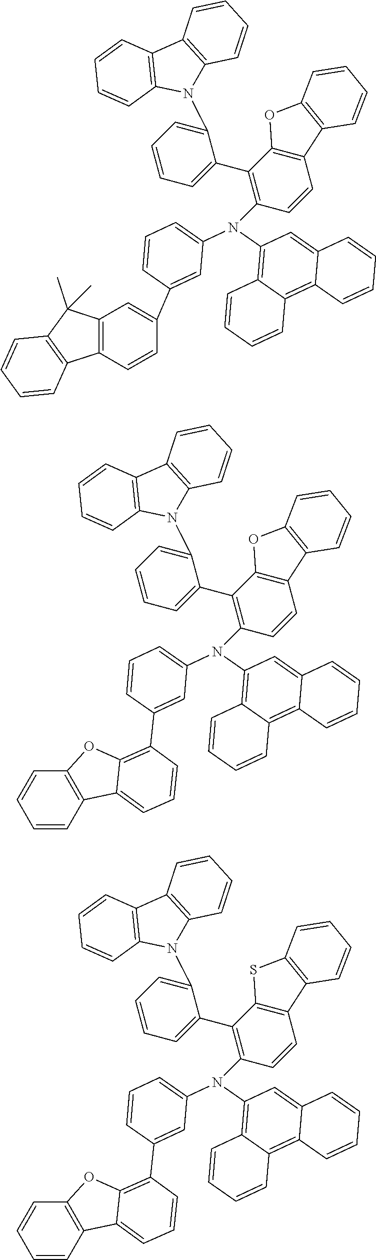 [SMILES: CC1(C)c2ccccc2-c2ccc(-c3cccc(N(c4ccc5c(oc6ccccc65)c4-c4ccccc4-n4c5ccccc5c5ccccc54)c4cc5ccccc5c5ccccc45)c3)cc21 ; c1cc(-c2cccc3c2oc2ccccc23)cc(N(c2ccc3c(oc4ccccc43)c2-c2ccccc2-n2c3ccccc3c3ccccc32)c2cc3ccccc3c3ccccc23)c1 ; c1cc(-c2cccc3c2oc2ccccc23)cc(N(c2ccc3c(sc4ccccc43)c2-c2ccccc2-n2c3ccccc3c3ccccc32)c2cc3ccccc3c3ccccc23)c1]

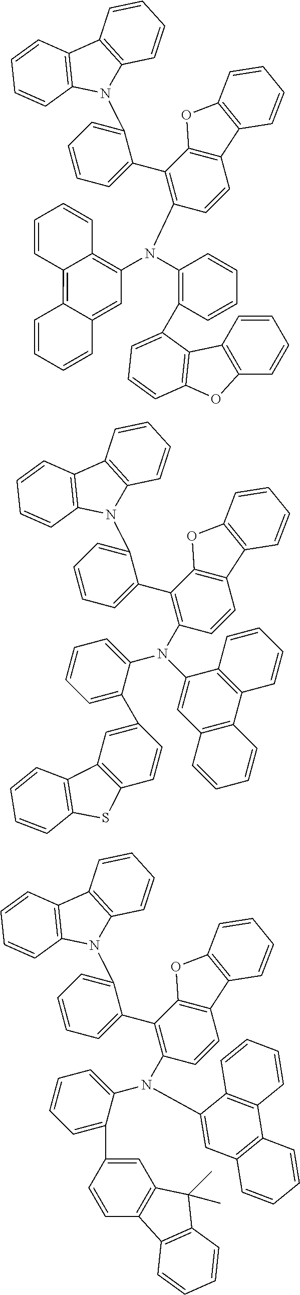

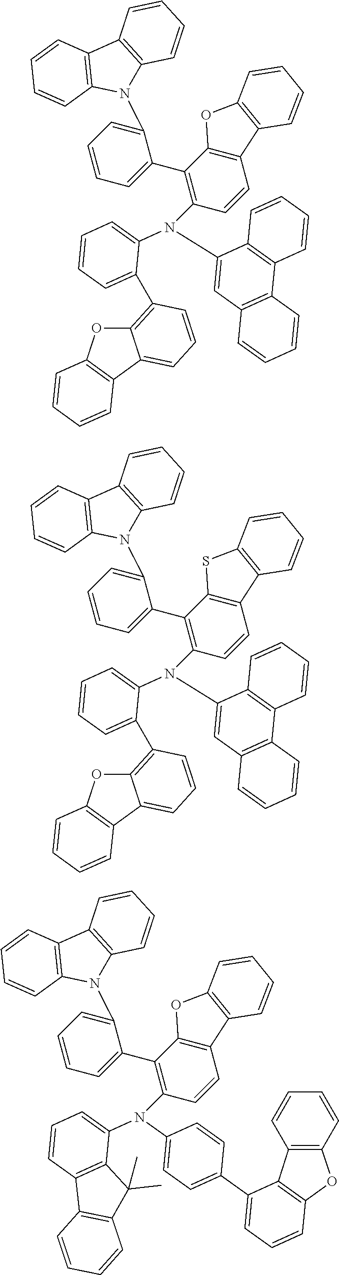

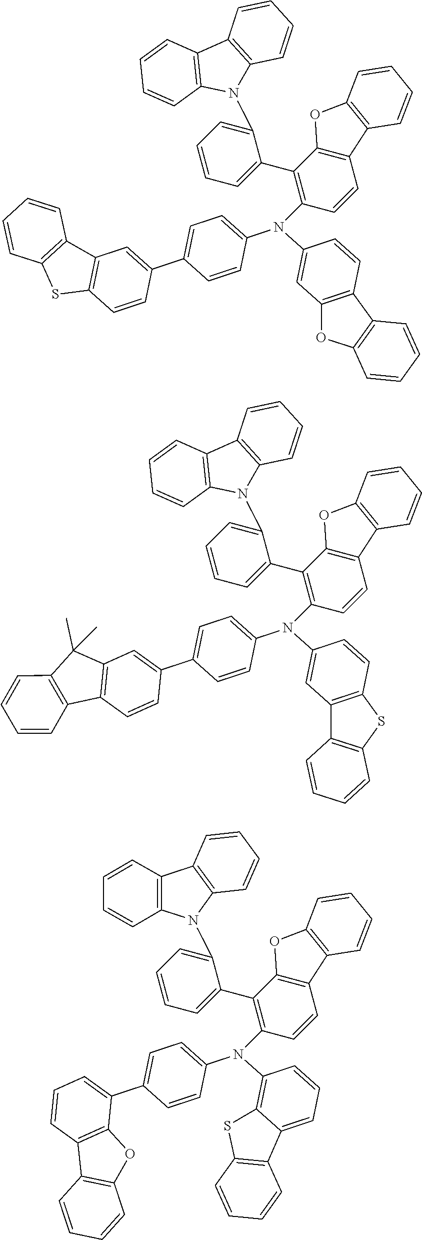 [SMILES: c1ccc(-n2c3ccccc3c3ccccc32)c(-c2c(N(c3ccc(-c4ccc5sc6ccccc6c5c4)cc3)c3ccc4c(c3)oc3ccccc34)ccc3c2oc2ccccc23)c1 ; CC1(C)c2ccccc2-c2ccc(-c3ccc(N(c4ccc5sc6ccccc6c5c4)c4ccc5c(oc6ccccc65)c4-c4ccccc4-n4c5ccccc5c5ccccc54)cc3)cc21 ; c1ccc(-n2c3ccccc3c3ccccc32)c(-c2c(N(c3ccc(-c4cccc5c4oc4ccccc45)cc3)c3cccc4c3sc3ccccc34)ccc3c2oc2ccccc23)c1]

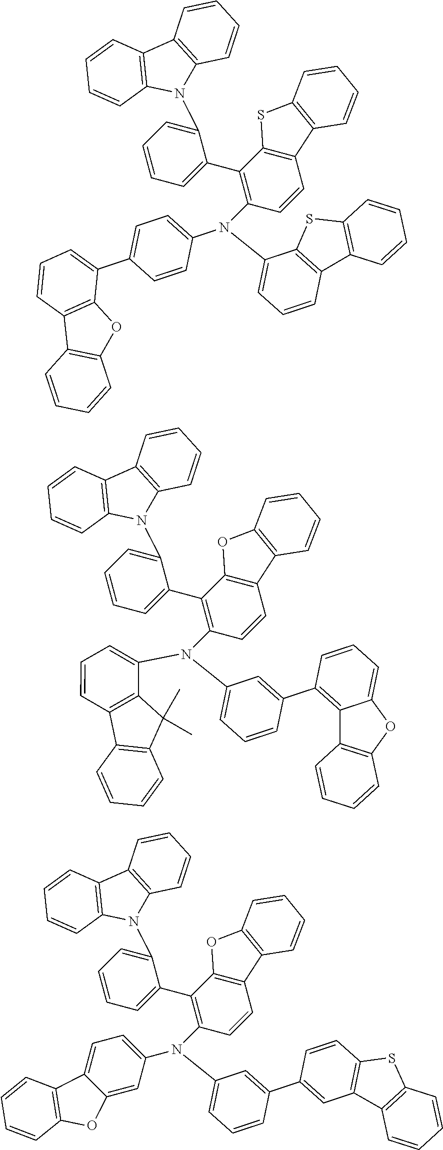

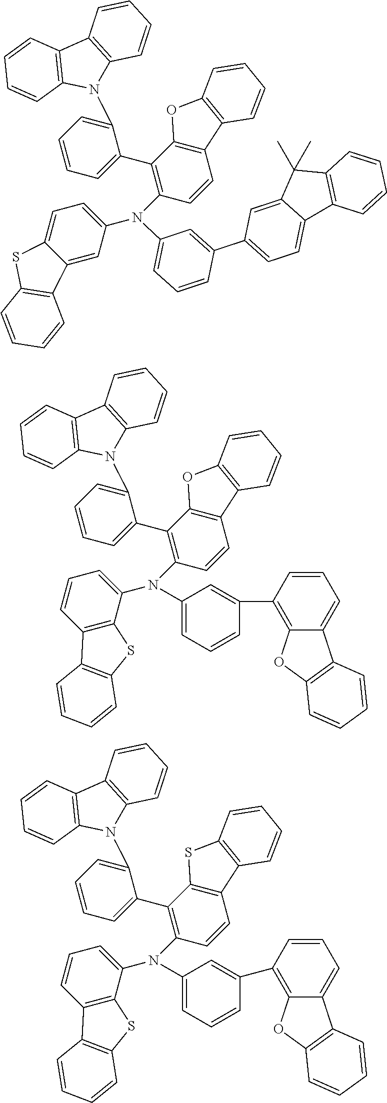

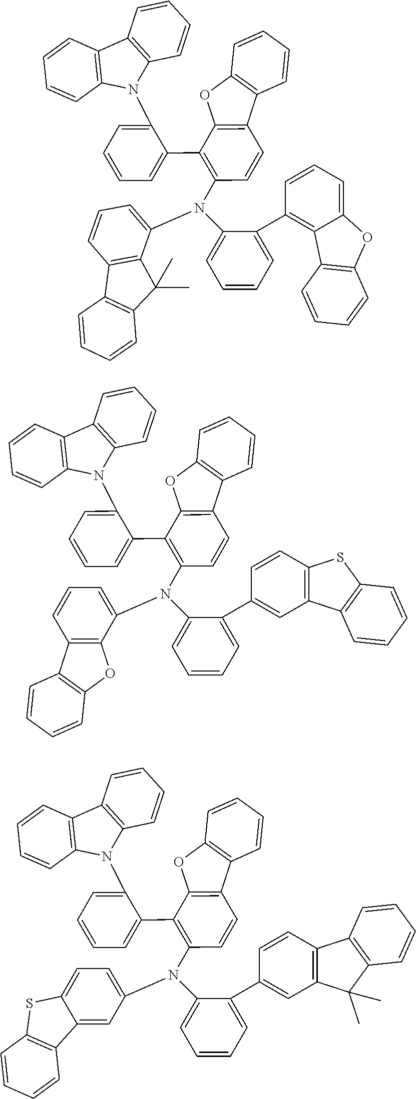

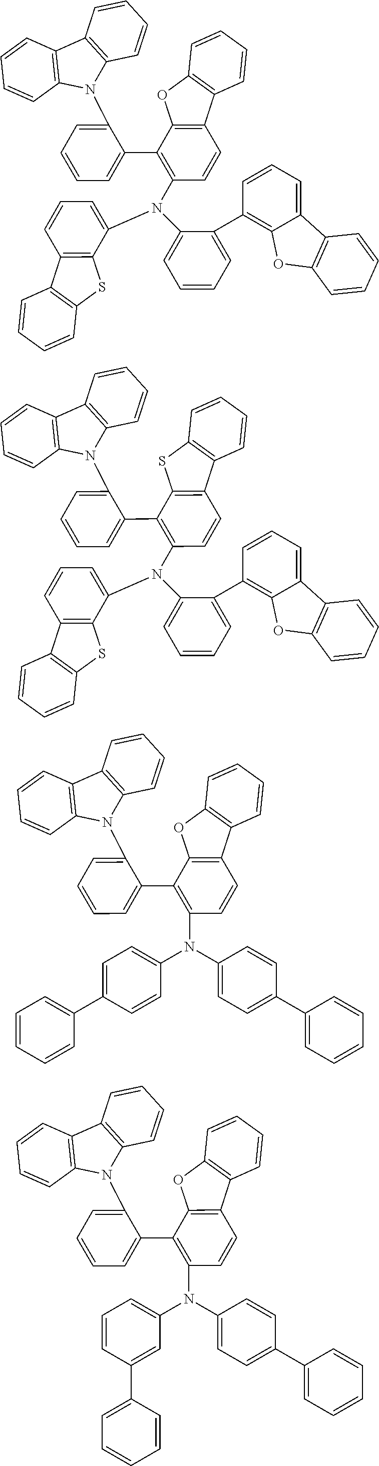

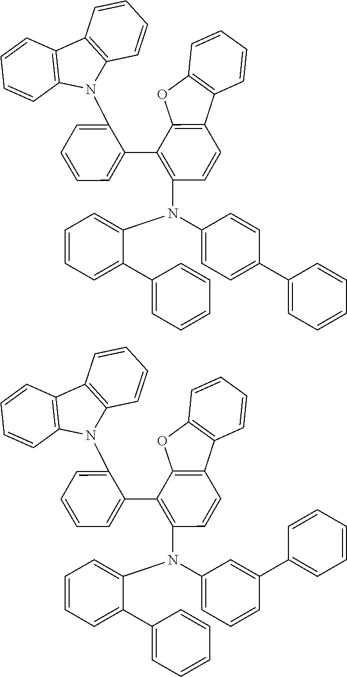

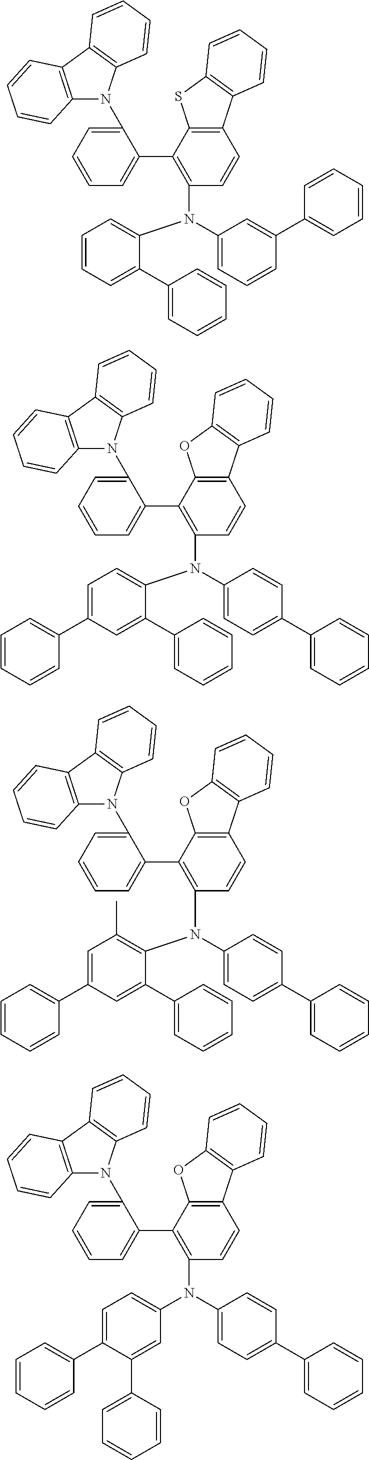 [SMILES: c1ccc(-c2cccc(N(c3ccccc3-c3ccccc3)c3ccc4c(sc5ccccc54)c3-c3ccccc3-n3c4ccccc4c4ccccc43)c2)cc1 ; c1ccc(-c2ccc(N(c3ccc(-c4ccccc4)cc3-c3ccccc3)c3ccc4c(oc5ccccc54)c3-c3ccccc3-n3c4ccccc4c4ccccc43)cc2)cc1 ; Cc1cc(-c2ccccc2)cc(-c2ccccc2)c1N(c1ccc(-c2ccccc2)cc1)c1ccc2c(oc3ccccc32)c1-c1ccccc1-n1c2ccccc2c2ccccc21 ; c1ccc(-c2ccc(N(c3ccc(-c4ccccc4)c(-c4ccccc4)c3)c3ccc4c(oc5ccccc54)c3-c3ccccc3-n3c4ccccc4c4ccccc43)cc2)cc1]

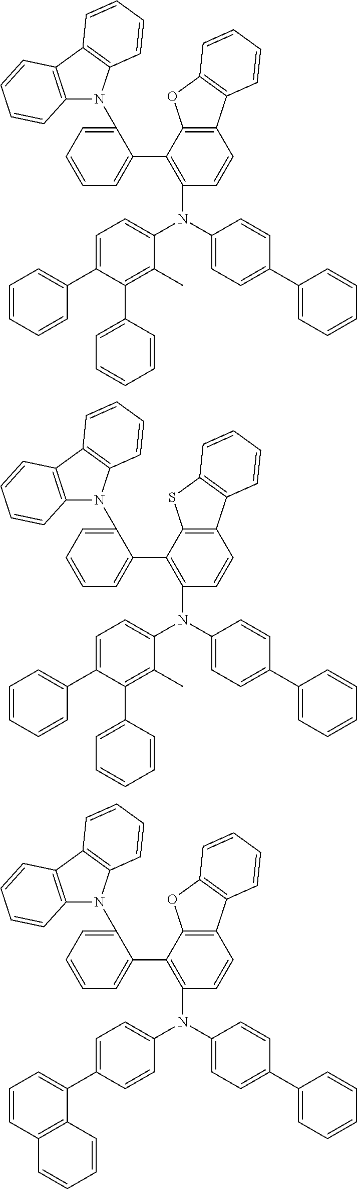

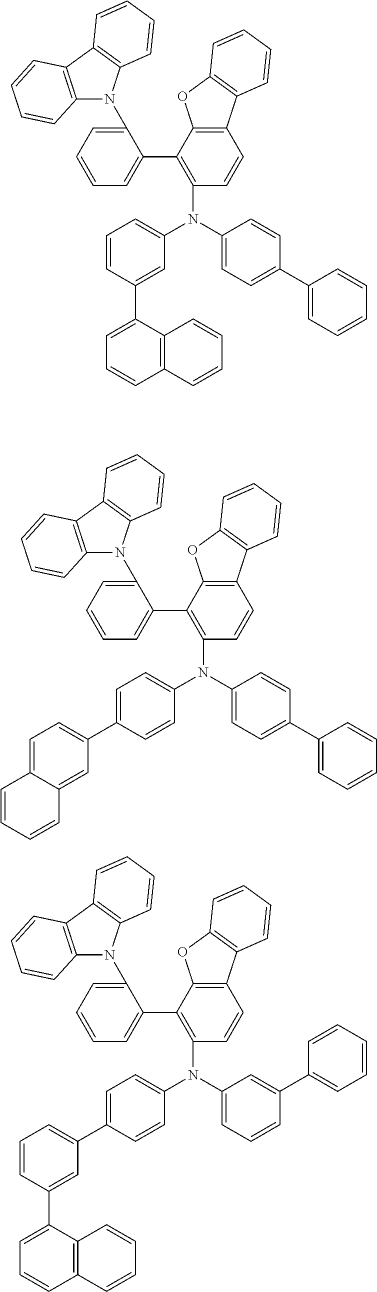 [SMILES: c1ccc(-c2ccc(N(c3cccc(-c4cccc5ccccc45)c3)c3ccc4c(oc5ccccc54)c3-c3ccccc3-n3c4ccccc4c4ccccc43)cc2)cc1 ; c1ccc(-c2ccc(N(c3ccc(-c4ccc5ccccc5c4)cc3)c3ccc4c(oc5ccccc54)c3-c3ccccc3-n3c4ccccc4c4ccccc43)cc2)cc1 ; c1ccc(-c2cccc(N(c3ccc(-c4cccc(-c5cccc6ccccc56)c4)cc3)c3ccc4c(oc5ccccc54)c3-c3ccccc3-n3c4ccccc4c4ccccc43)c2)cc1]

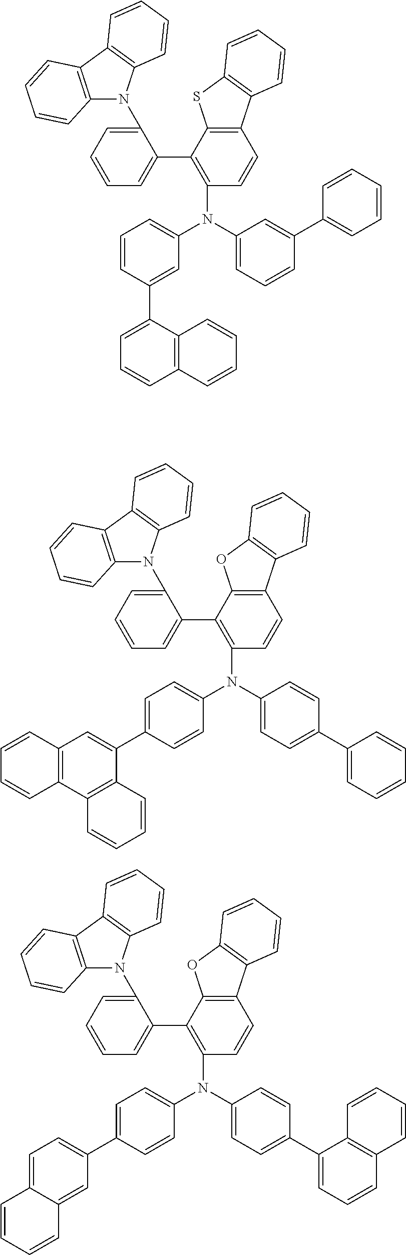

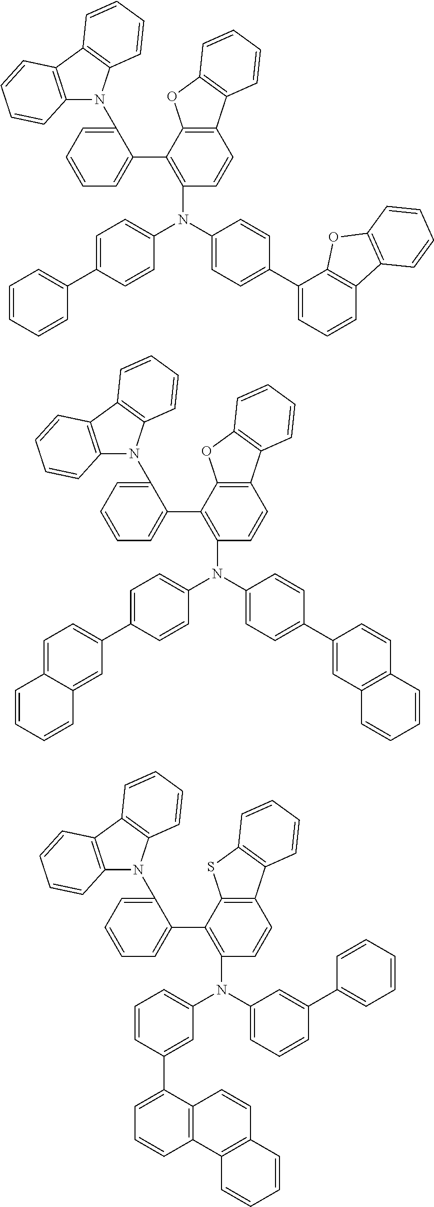 [SMILES: c1ccc(-c2ccc(N(c3ccc(-c4cccc5c4oc4ccccc45)cc3)c3ccc4c(oc5ccccc54)c3-c3ccccc3-n3c4ccccc4c4ccccc43)cc2)cc1 ; c1ccc(-n2c3ccccc3c3ccccc32)c(-c2c(N(c3ccc(-c4ccc5ccccc5c4)cc3)c3ccc(-c4ccc5ccccc5c4)cc3)ccc3c2oc2ccccc23)c1 ; c1ccc(-c2cccc(N(c3cccc(-c4cccc5c4ccc4ccccc45)c3)c3ccc4c(sc5ccccc54)c3-c3ccccc3-n3c4ccccc4c4ccccc43)c2)cc1]

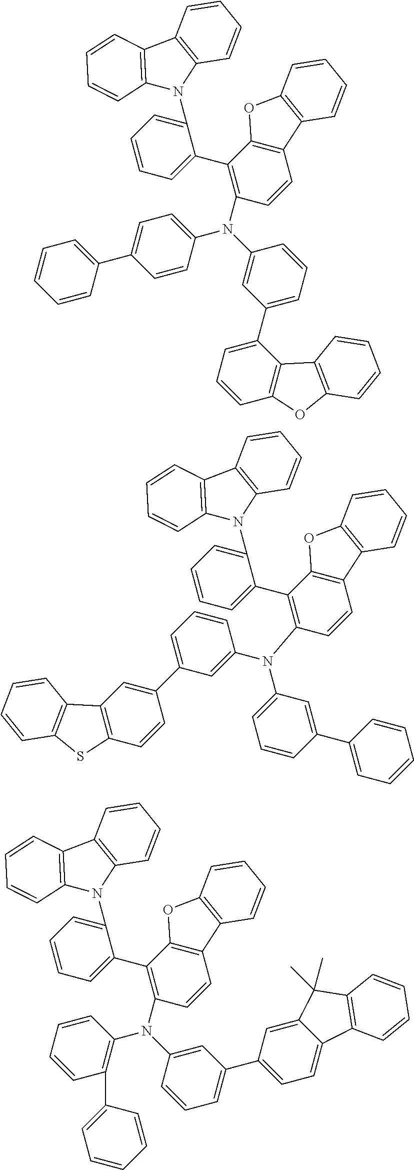

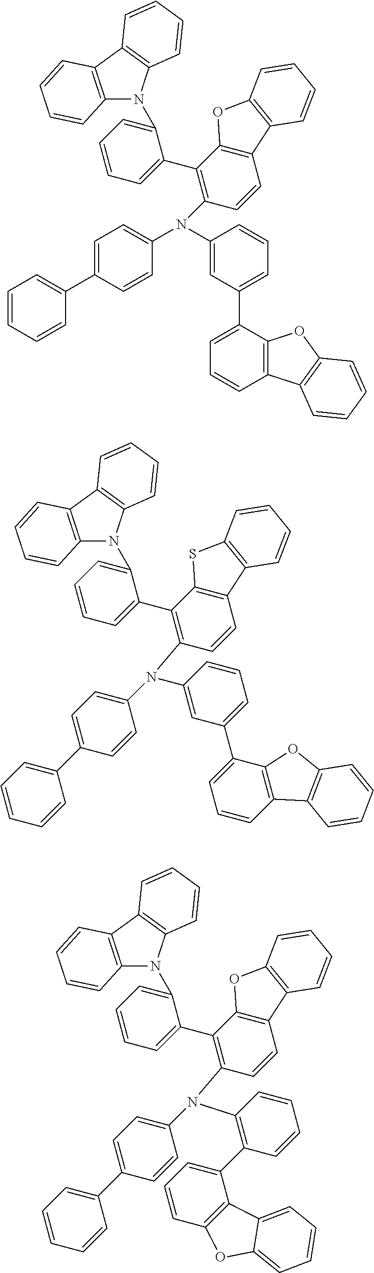 [SMILES: c1ccc(-c2ccc(N(c3cccc(-c4cccc5c4oc4ccccc45)c3)c3ccc4c(oc5ccccc54)c3-c3ccccc3-n3c4ccccc4c4ccccc43)cc2)cc1 ; c1ccc(-c2ccc(N(c3cccc(-c4cccc5c4oc4ccccc45)c3)c3ccc4c(sc5ccccc54)c3-c3ccccc3-n3c4ccccc4c4ccccc43)cc2)cc1 ; c1ccc(-c2ccc(N(c3ccccc3-c3cccc4oc5ccccc5c34)c3ccc4c(oc5ccccc54)c3-c3ccccc3-n3c4ccccc4c4ccccc43)cc2)cc1]

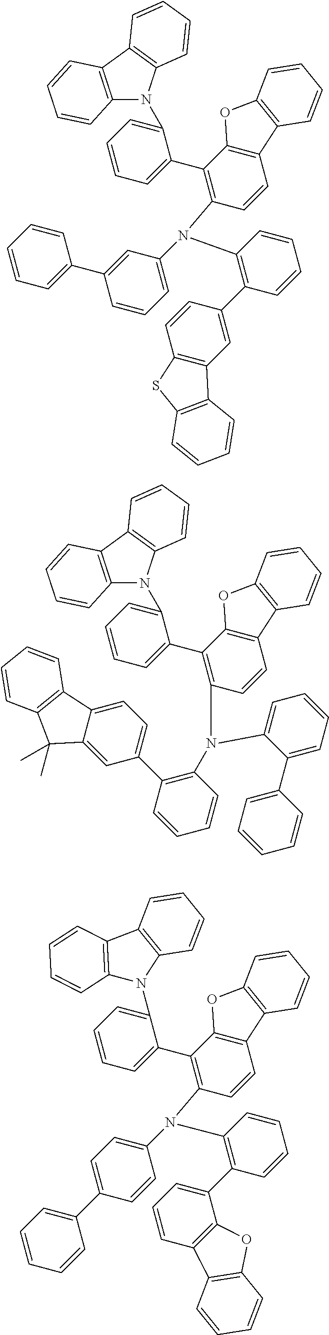

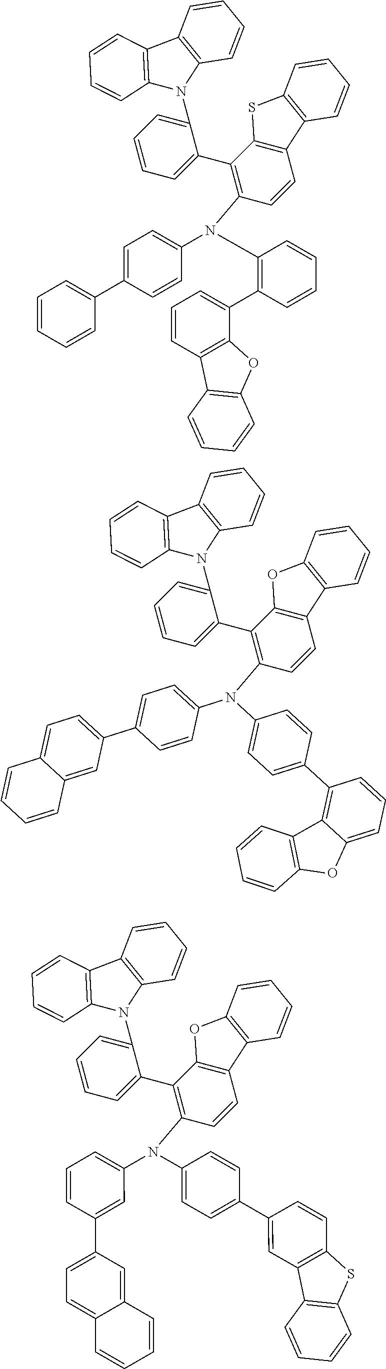

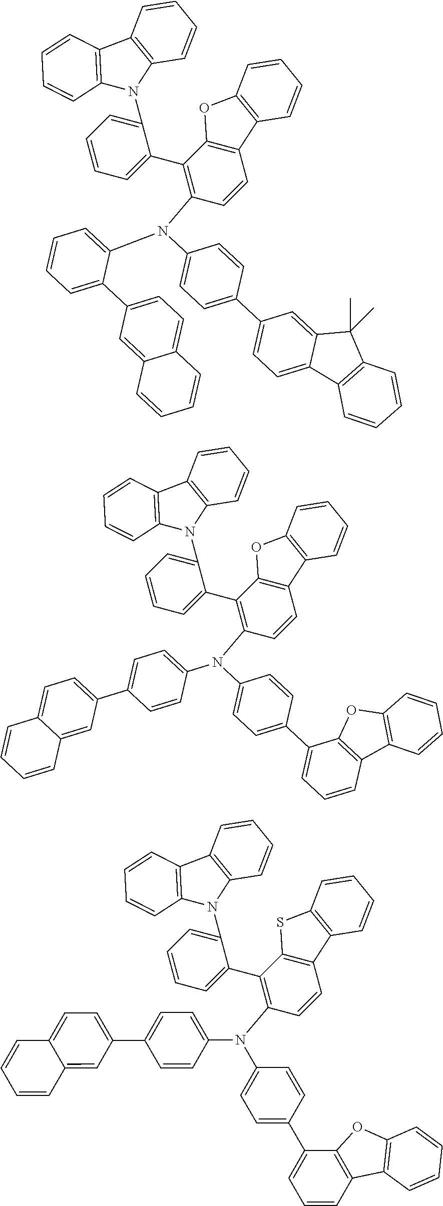 [SMILES: CC1(C)c2ccccc2-c2ccc(-c3ccc(N(c4ccccc4-c4ccc5ccccc5c4)c4ccc5c(oc6ccccc65)c4-c4ccccc4-n4c5ccccc5c5ccccc54)cc3)cc21 ; c1ccc(-n2c3ccccc3c3ccccc32)c(-c2c(N(c3ccc(-c4ccc5ccccc5c4)cc3)c3ccc(-c4cccc5c4oc4ccccc45)cc3)ccc3c2oc2ccccc23)c1 ; c1ccc(-n2c3ccccc3c3ccccc32)c(-c2c(N(c3ccc(-c4ccc5ccccc5c4)cc3)c3ccc(-c4cccc5c4oc4ccccc45)cc3)ccc3c2sc2ccccc23)c1]

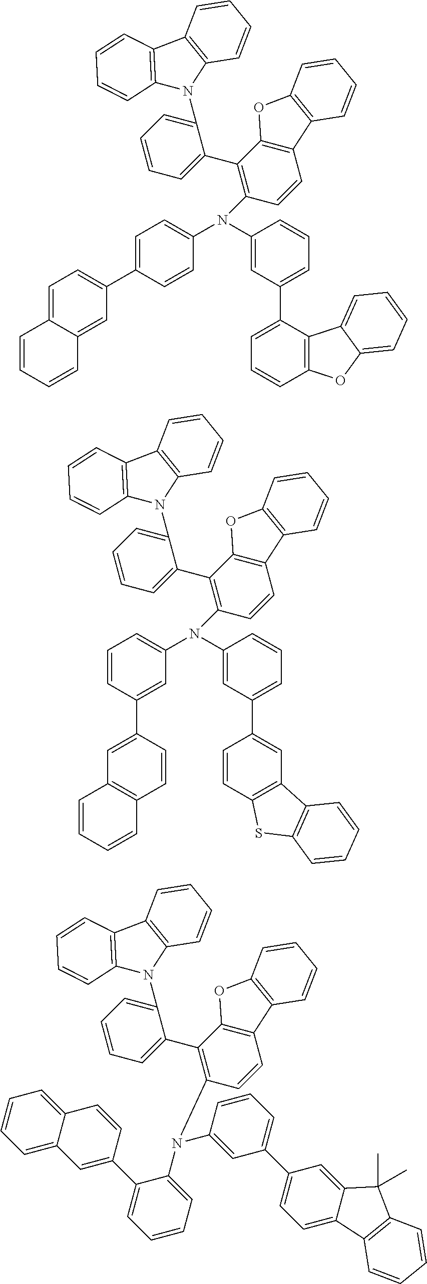 [SMILES: c1cc(-c2cccc3oc4ccccc4c23)cc(N(c2ccc(-c3ccc4ccccc4c3)cc2)c2ccc3c(oc4ccccc43)c2-c2ccccc2-n2c3ccccc3c3ccccc32)c1 ; c1cc(-c2ccc3ccccc3c2)cc(N(c2cccc(-c3ccc4sc5ccccc5c4c3)c2)c2ccc3c(oc4ccccc43)c2-c2ccccc2-n2c3ccccc3c3ccccc32)c1 ; CC1(C)c2ccccc2-c2ccc(-c3cccc(N(c4ccccc4-c4ccc5ccccc5c4)c4ccc5c(oc6ccccc65)c4-c4ccccc4-n4c5ccccc5c5ccccc54)c3)cc21]

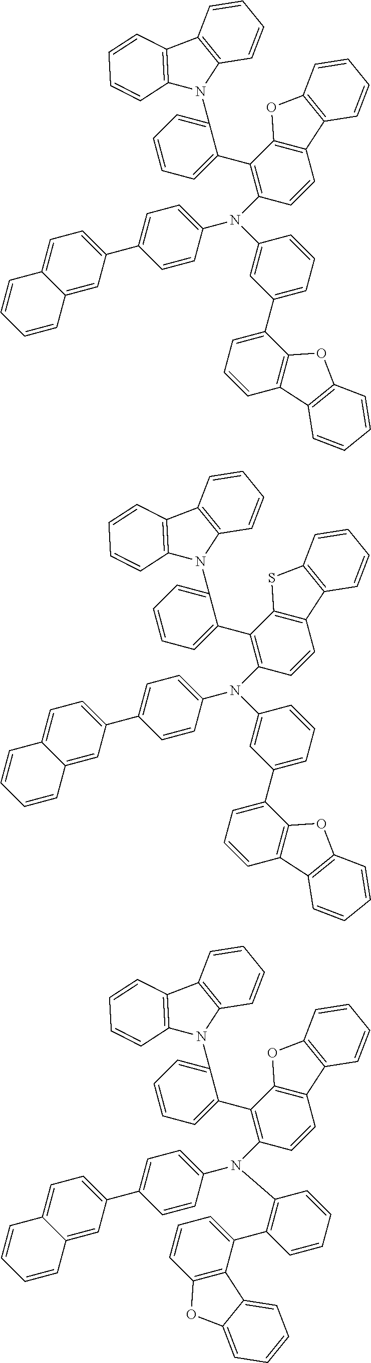 [SMILES: c1cc(-c2cccc3c2oc2ccccc23)cc(N(c2ccc(-c3ccc4ccccc4c3)cc2)c2ccc3c(oc4ccccc43)c2-c2ccccc2-n2c3ccccc3c3ccccc32)c1 ; c1cc(-c2cccc3c2oc2ccccc23)cc(N(c2ccc(-c3ccc4ccccc4c3)cc2)c2ccc3c(sc4ccccc43)c2-c2ccccc2-n2c3ccccc3c3ccccc32)c1 ; c1ccc(N(c2ccc(-c3ccc4ccccc4c3)cc2)c2ccc3c(oc4ccccc43)c2-c2ccccc2-n2c3ccccc3c3ccccc32)c(-c2cccc3oc4ccccc4c23)c1]

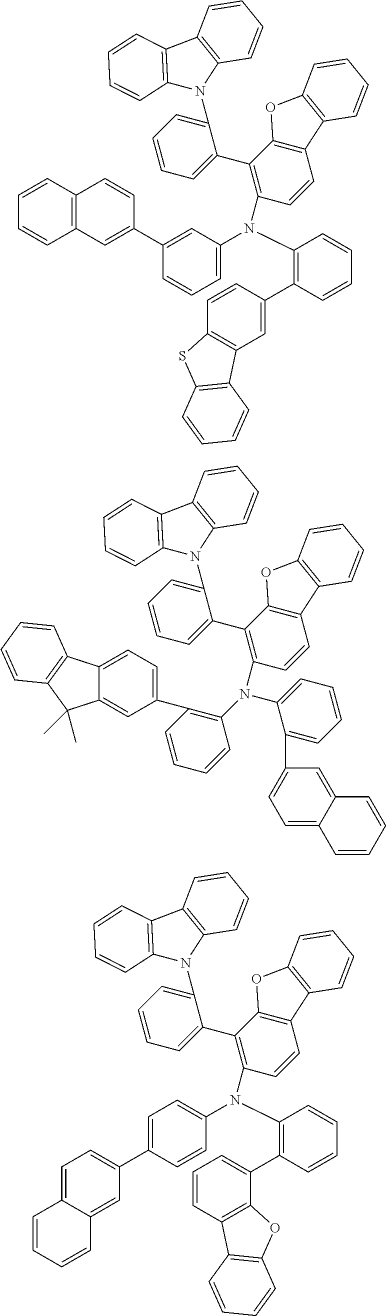

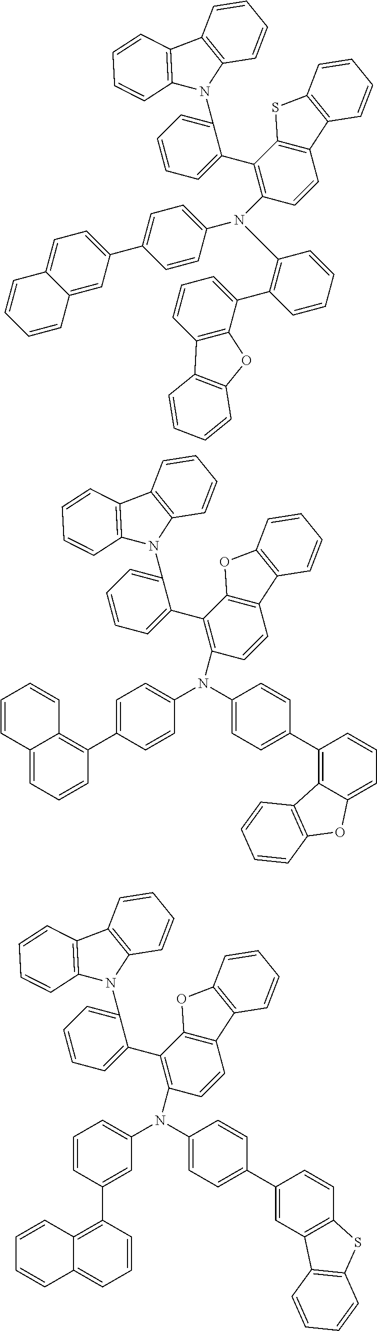

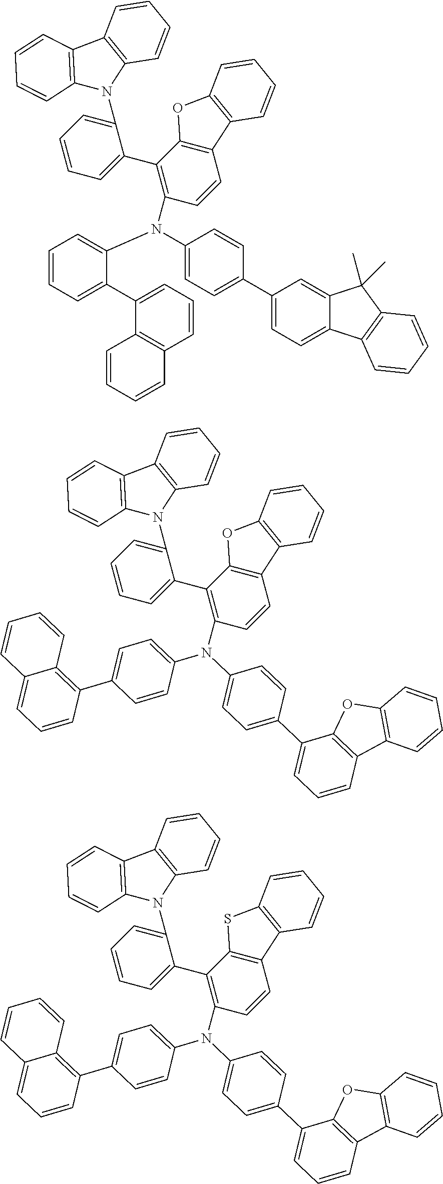

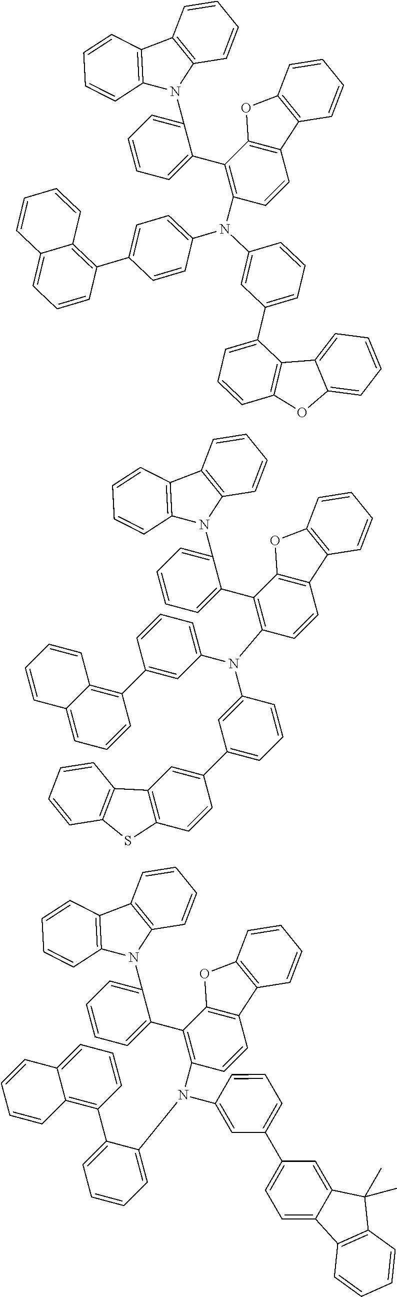

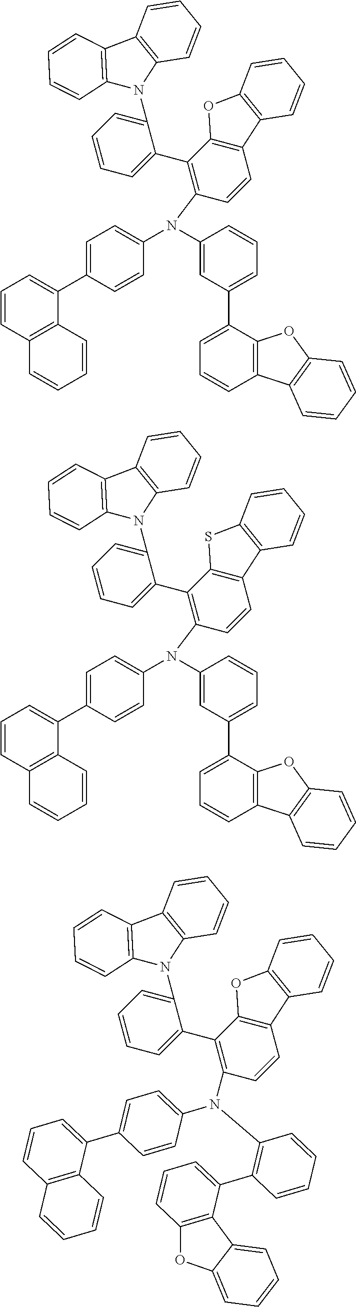 [SMILES: c1cc(-c2cccc3c2oc2ccccc23)cc(N(c2ccc(-c3cccc4ccccc34)cc2)c2ccc3c(oc4ccccc43)c2-c2ccccc2-n2c3ccccc3c3ccccc32)c1 ; c1cc(-c2cccc3c2oc2ccccc23)cc(N(c2ccc(-c3cccc4ccccc34)cc2)c2ccc3c(sc4ccccc43)c2-c2ccccc2-n2c3ccccc3c3ccccc32)c1 ; c1ccc(N(c2ccc(-c3cccc4ccccc34)cc2)c2ccc3c(oc4ccccc43)c2-c2ccccc2-n2c3ccccc3c3ccccc32)c(-c2cccc3oc4ccccc4c23)c1]

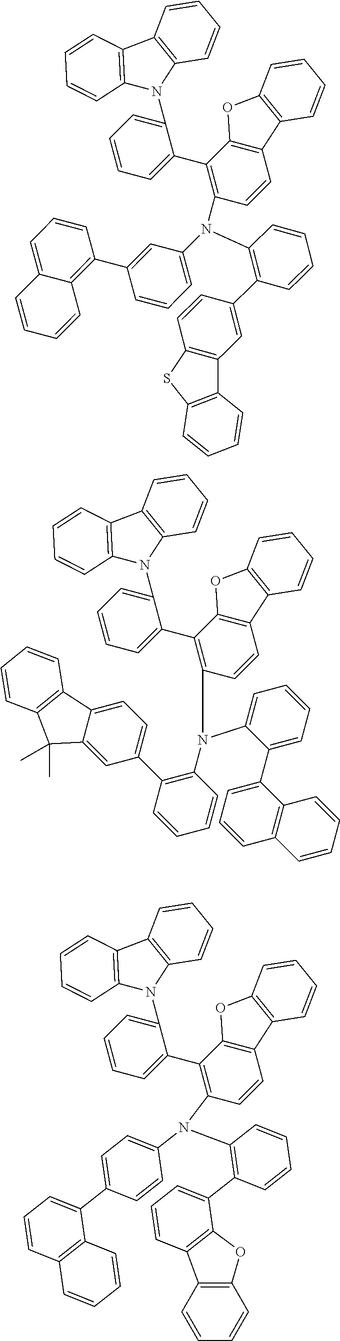

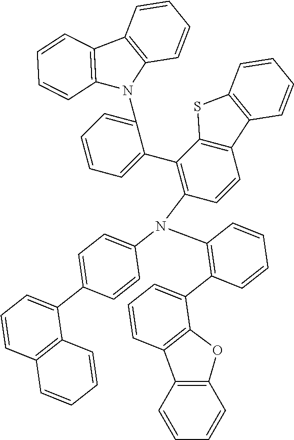 [SMILES: c1ccc(N(c2ccc(-c3cccc4ccccc34)cc2)c2ccc3c(sc4ccccc43)c2-c2ccccc2-n2c3ccccc3c3ccccc32)c(-c2cccc3c2oc2ccccc23)c1]

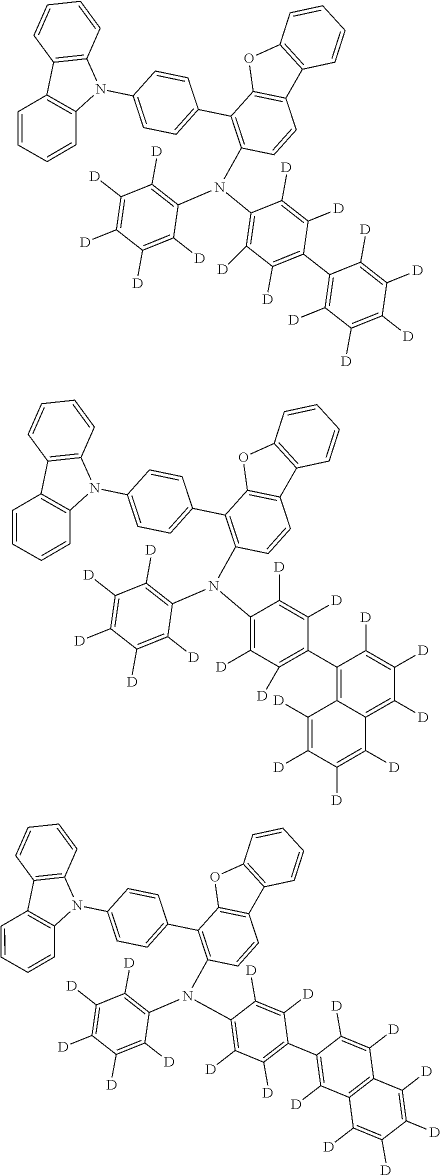

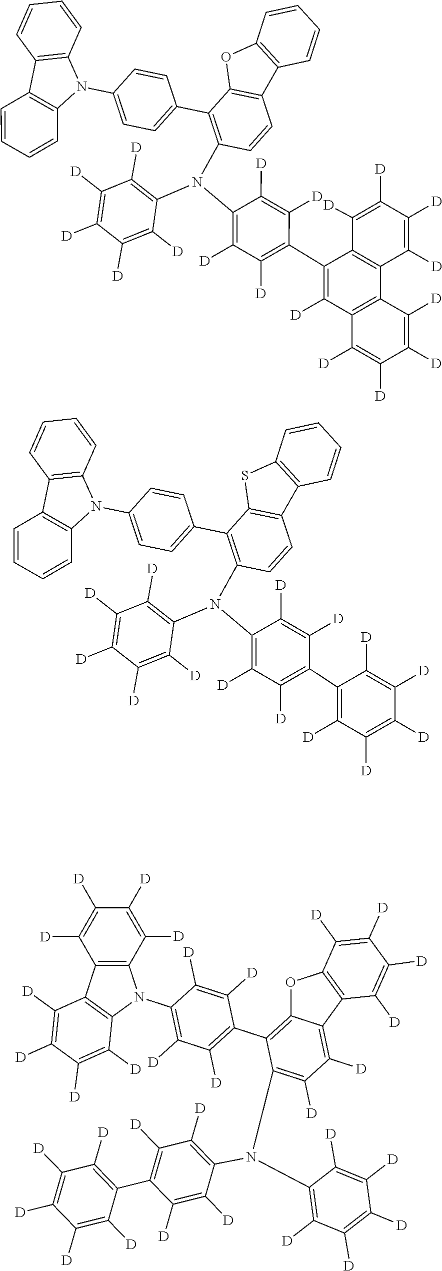

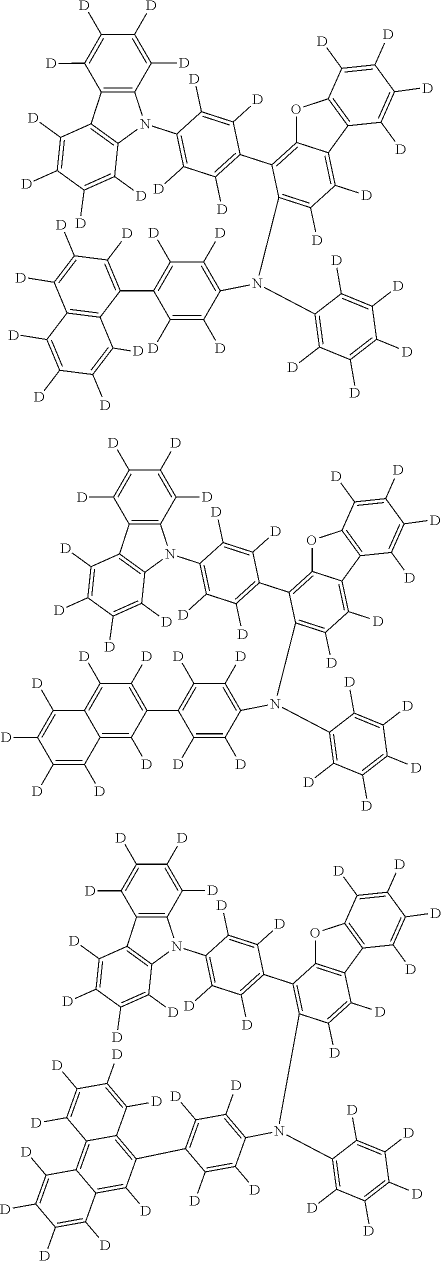

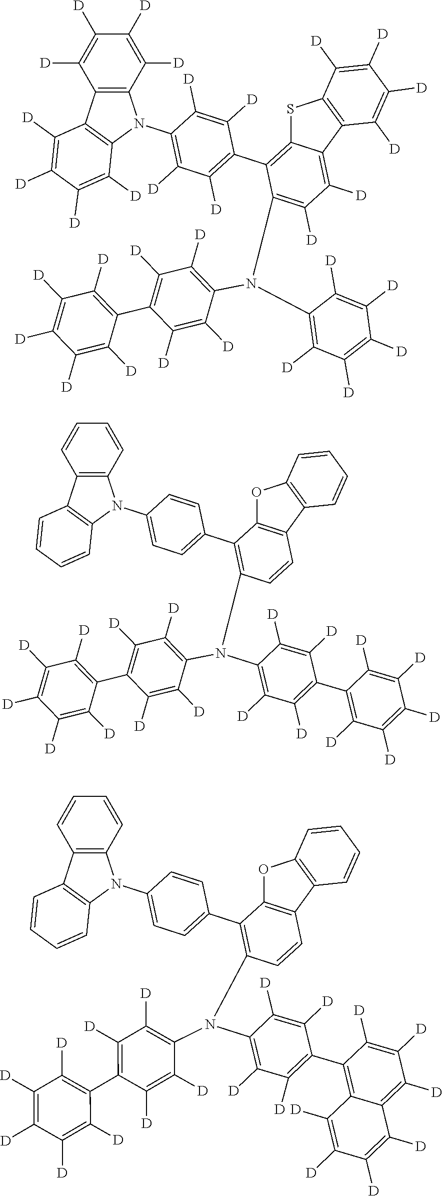

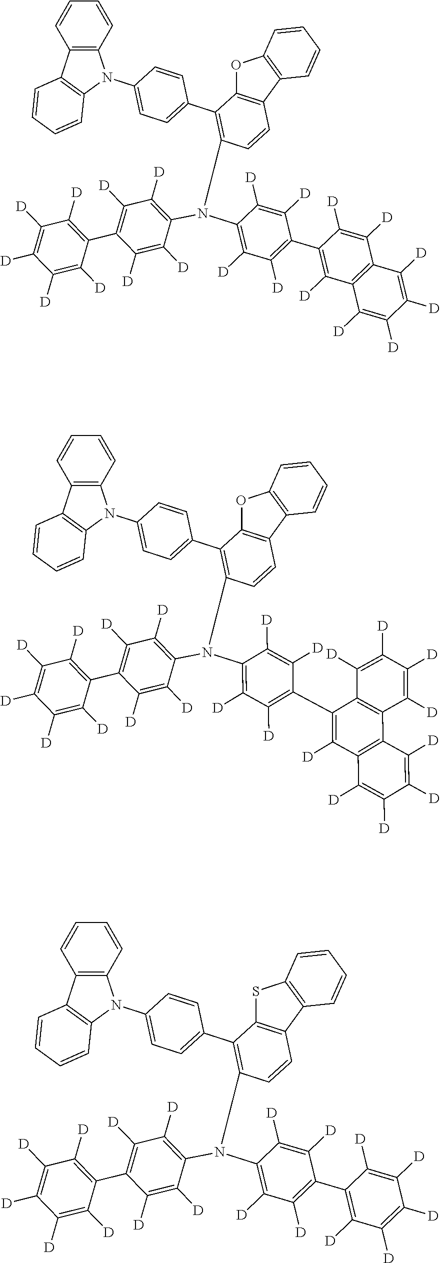

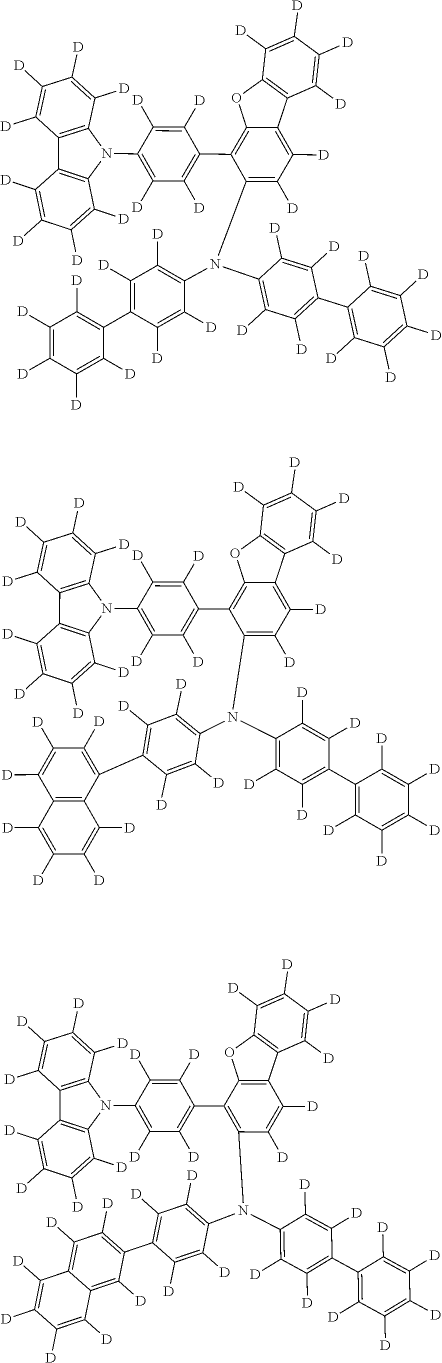

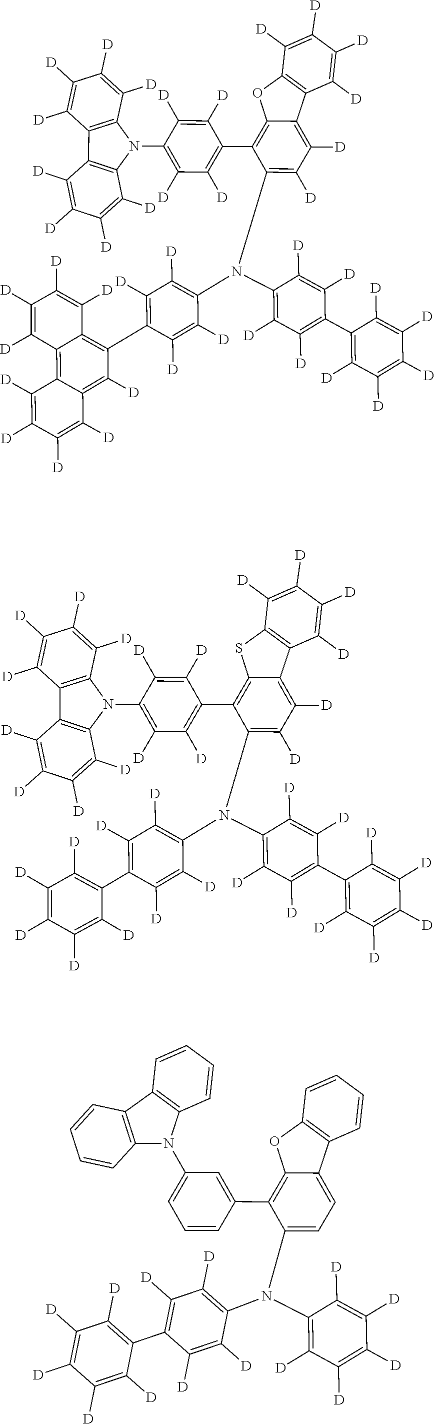

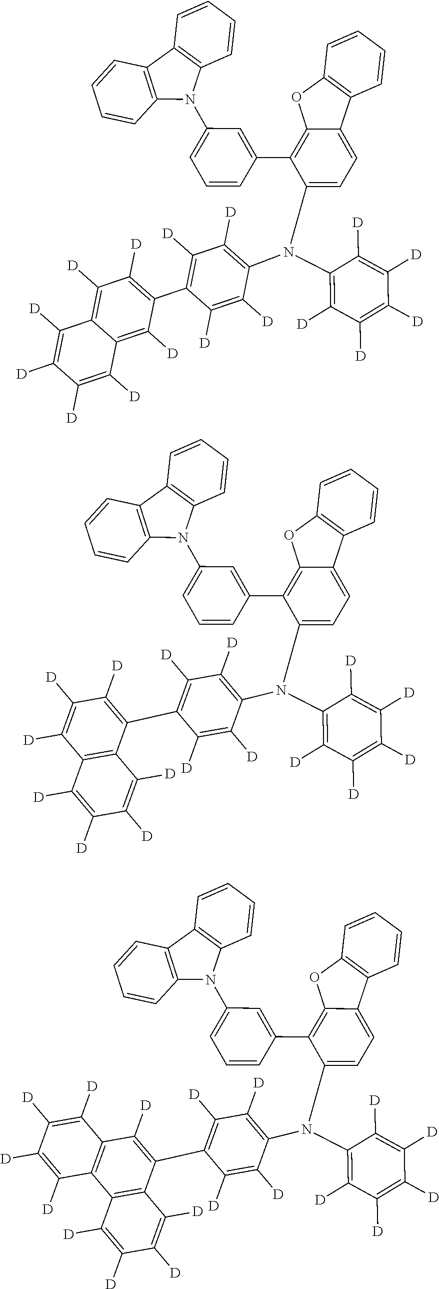

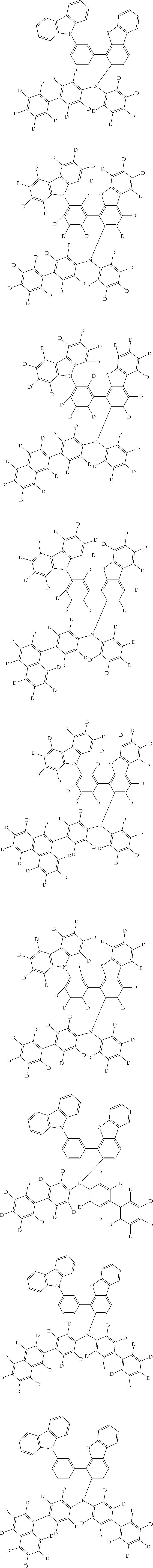

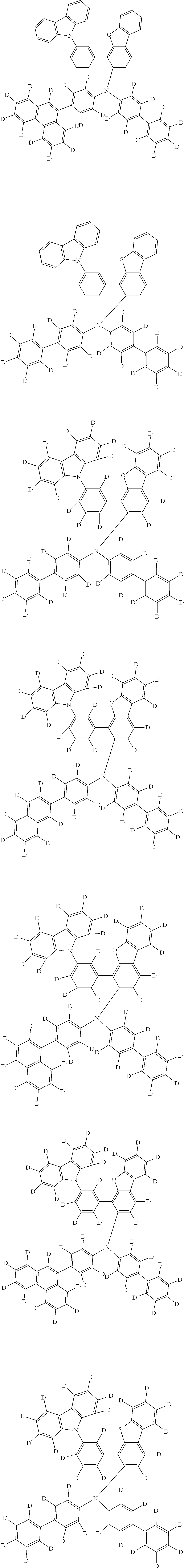

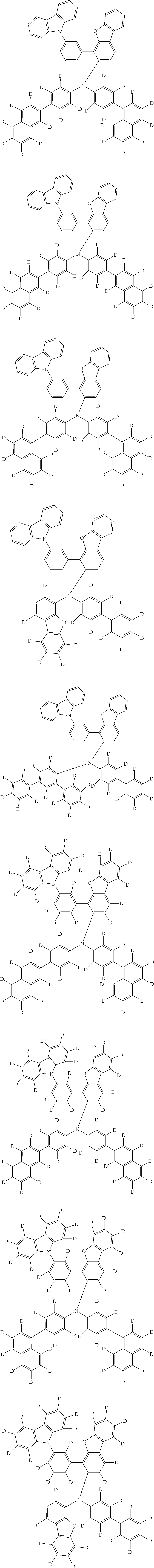

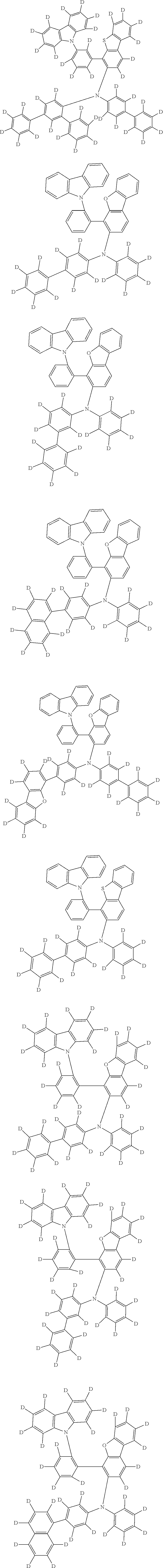

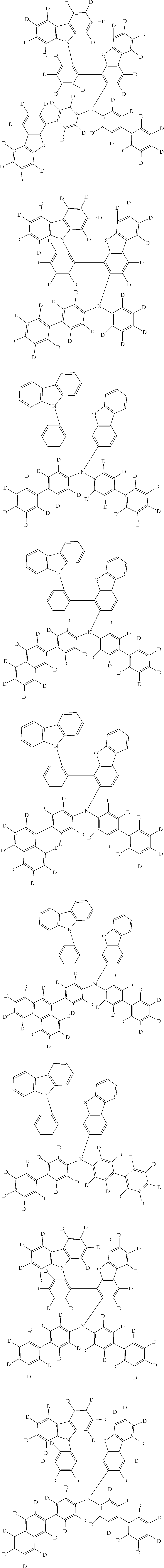

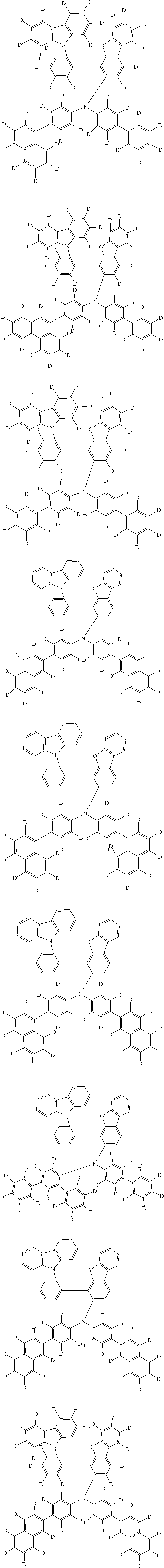

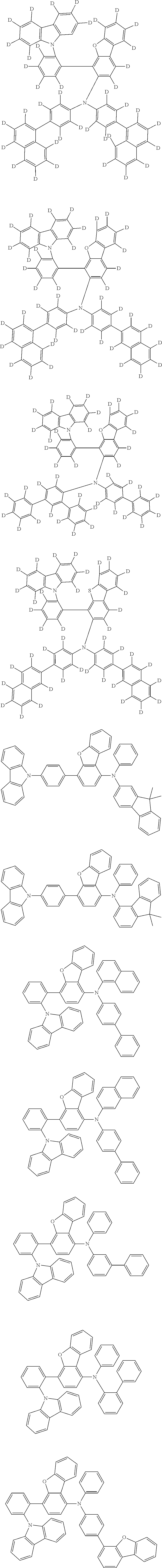

The compound of Chemical Formula 1 of the present invention may be usefully used as a material for a hole transport auxiliary layer.

The compound of Chemical Formula 1 in one embodiment has an amine group bonded to one side and a carbazole group substituted with an aryl group bonded to the other side with respect to dibenzofuran or dibenzothiophene, and thus when used as a material for an organic electroluminescent device, can show equal or excellent characteristics in most device characteristics, such as luminous efficiency and lifetime.

The compound of Chemical Formula 1 in another embodiment has an amine group bonded to one side at any one of positions 1 to 3 of dibenzofuran or dibenzothiophene and a carbazole group substituted with an aryl group bonded to the other side at position 4 of dibenzofuran or dibenzothiophene, and thus when used as a material for an organic electroluminescent device, can show equal or excellent characteristics in most device characteristics, such as luminous efficiency and lifetime.

The carbon positions of the dibenzofuran or dibenzothiophene are as follows.

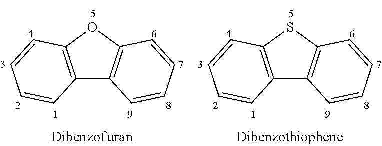

The present invention provides an organic electroluminescent device containing the compound represented by Chemical Formula 1.

The organic compound of the present invention may be usefully used as a material for a hole transport auxiliary layer.

In addition, according to the present invention, in an organic electroluminescent device in which an organic thin film layer including one or more layers including at least a light emitting layer is stacked between a positive electrode and a negative electrode, the organic thin film layer is a hole transport auxiliary layer between the first electrode and the light emitting layer.

The hole transport auxiliary layer may comprise the compound represented by Chemical Formula 1.

The hole transport auxiliary layer may adjust hole injection characteristics by reducing a difference in HOMO energy level between the hole transport layer and the light emitting layer, thereby reducing the accumulation of holes at an interface between the hole transport auxiliary layer and the light emitting layer to reduce a quenching phenomenon in which excitons extinct due to polarons at the interface. Therefore, it is possible to reduce a degradation phenomenon of the device, thereby stabilizing the device and increasing efficiency and lifetime.

The organic electroluminescent device may have a structure in which a positive electrode, a hole injection layer, a hole transport layer, a hole transport auxiliary layer, a light emitting layer, an electron transport layer, an electron injection layer, and a negative electrode are stacked, and an electron transport auxiliary layer may be added if necessary.

Hereinafter, the organic electroluminescent device of the present invention will be described as an example. However, the contents illustrated below do not limit the organic electroluminescent device of the present invention.

The organic electroluminescent device of the present invention may have a structure in which a positive electrode (hole injection electrode), a hole injection layer (HIL), a hole transport layer (HTL), a hole transport auxiliary layer, a light emitting layer (EML), and a negative electrode (electron injection electrode) are sequentially stacked and preferably, may further include a hole transport auxiliary layer between the positive electrode and the light emitting layer, and a hole transport auxiliary layer between the positive electrode and the light emitting layer, and electron transport layer (ETL) and an electron injection layer (EIL) between the negative electrode and the light emitting layer. In addition, an electron transport auxiliary layer may be further included between the negative electrode and the light emitting layer.

In a method of manufacturing an organic electroluminescent device according to the present invention, first, a positive electrode is formed by coating a surface of a substrate with a material for a positive electrode in a typical method. In this case, the substrate used is preferably a glass substrate or a transparent plastic substrate with excellent transparency, surface smoothness, ease of handling, and waterproofness. In addition, indium tin oxide (ITO), indium zinc oxide (IZO), tin oxide (SnO₂), zinc oxide (ZnO), and the like, which are transparent and have excellent conductivity, may be used as a material for a positive electrode.

Next, a hole injection layer is formed by forming a material for a hole injection layer (HIL) on a surface of the positive electrode by vacuum thermal evaporation or spin coating in a typical method. Examples of the material for a hole injection layer may include copper phthalocyanine (CuPc), 4,4′,4″-tris(3-methylphenylamino)triphenylamine (m-MTDATA), 4,4′,4″-tris(3-methylphenyamino)phenoxybenzene (m-MTDAPB), 4,4′,4″-tri(N-carbazolyl)triphenylamine (TCTA) which is starburst type amines, 4,4′,4″-tris(N-(2-naphthyl)-N-phenylamino)-triphenylamine(2-TNATA), or IDE406 available from Idemitsu.

A hole transport layer is formed by forming a material for a hole transport layer on a surface of the hole injection layer by vacuum thermal evaporation or spin coating in a typical method. As the material for a hole transport layer, a commonly used material for a hole transport layer may be used.

A hole transport auxiliary layer may be formed by forming the compound represented by Chemical Formula 1 according to the present invention on a surface of the hole transport layer by vacuum thermal evaporation or spin coating. As described above, in the hole transport auxiliary layer, the compound according to the present invention may be used as the material for a hole transport auxiliary layer, and a commonly used material for a hole transport auxiliary layer may be used to form the hole transport auxiliary layer.

A light emitting layer is formed by forming a material for a light emitting layer (EML) on a surface of the hole transport auxiliary layer by vacuum thermal evaporation or spin coating in a typical method. In this case, a sole light emitting material or light emitting host material among materials for a light emitting layer used may use Tris(8-hydroxyquinolinato)aluminium (Alq₃) or the like for green and use Alq₃, CBP(4,4′-N,N′-dicabazole-biphenyl), PVK(poly(n-vinylcabazole)), ADN(9, 10-di(naphthalene-2-yl)anthracene), TCTA, TPBI(1,3,5-tris(N-phenylbenzimidazole-2-yl)benzene), TBADN(3-tert-butyl-9,10-di(naphth-2-yl)anthracene), E3, DSA(distyrylarylene), or a mixture of two or more thereof for blue, but the present invention is not limited thereto.

In the case of dopant which may be used together with a light emitting host among the materials for a light emitting layer, IDE102 and IDE105 available from Idemitsu company may be used, and as a phosphorescent dopant, tris(2-phenylpyridine)iridium(III)(Ir(ppy)₃), iridium(III)bis[(4,6-difluorophenyl)pyridinato-N,C-2′]picolinate (FIrpic) (reference [Chihaya Adachi, et al., Appl. Phys. Lett., 2001, 79, 2082-2084]), platinium(II)octaethylporphyrin(PtOEP), TBE002 (by Covion company), or the like may be used.

An electron transport layer is formed by forming a material for the electron transport layer (ETL) on a surface of the light emitting layer by vacuum thermal evaporation or spin coating in a typical method. In this case, the material for an electron transport layer used is not especially limited, and Tris(8-hydroxyquinolinato)aluminium(Alq₃) may be preferably used.

Optionally, by additionally forming a hole blocking layer (HBL) between the light emitting layer and the electron transport layer and using the phosphorescent dopant in the light emitting layer, it is possible to prevent triplet excitons or holes from diffusing into the electron transport layer.

The formation of the hole blocking layer may be performed by vacuum thermal evaporation and spin coating of a material for a hole blocking layer in a typical method, and the material for a hole blocking layer is not especially limited, but can preferably use 8-Hydroxyquinolinolato-lithium (Liq), Bis(8-hydroxy-2-methylquinoline)-(4-phenylphenoxy)aluminum (BAlq), bathocuproine(BCP), LiF, or the like.

An electron injection layer is formed by forming a material for an electron injection layer (EIL) on a surface of the electron transport layer by vacuum thermal evaporation or spin coating in a typical method. In this case, materials such as LiF, Liq, Li₂O, BaO, NaCl, and CsF may be used as the material for an electron injection layer.

A negative electrode is formed by forming a negative electrode material on a surface of the electron injection layer by vacuum thermal deposition in a typical method.

In this case, as the negative electrode material used, lithium (Li), aluminum (Al), aluminum-lithium (Al—Li), calcium (Ca), magnesium (Mg), magnesium-indium (Mg—In), and magnesium-silver (Mg—Ag), and the like may be used. In addition, in the case of a top-emitting organic electroluminescent device, a transparent negative electrode through which light may transmit may be formed by using indium tin oxide (ITO) or indium zinc oxide (IZO).

A capping layer CPL may be formed on a surface of the negative electrode using a composition for the formation of the capping layer.

Hereinafter, a method of synthesizing the compounds will be described below as a representative example. However, the method of synthesizing the compounds of the present invention is not limited to the method exemplified below, and the compounds of the present invention may be manufactured by the methods exemplified below and methods known in the art.

### Synthesis Example

### 1. Synthesis of SUB 1

SUB 1 may be synthesized as follows, but is not limited thereto.

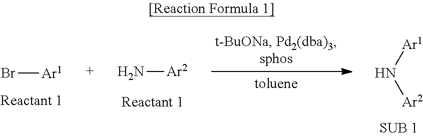

Reactant 1 (89.64 mmol), Reactant 2 (94.12 mmol), t-BuONa (179.28 mmol), Pd₂(dba)₃ (1.79 mmol), sphos (3.58 mmol), and toluene were added, stirred, and refluxed in a 500 mL flask under nitrogen air flow. After the reaction was finished, an organic layer was extracted by using toluene and water. The extracted solution was treated with MgSO₄ to remove remaining moisture, concentrated under reduced pressure, purified by using a column chromatography method, and then recrystallized to obtain SUB 1.

In addition, Ar₁ and Ar₂ substituted with deuterium may be synthesized in the same manner as Reaction Formula 1. The synthesized result of SUB1 is expressed by Table 1 below.

TABLE 1

| | | | | Obtained | |
|---|---|---|---|---|---|
| | | | | amount | |
| Item | Reactant 1 | Reactant 2 | SUB 1 | (Yield) | [M + H]⁺ |
| SUB 1-1 | 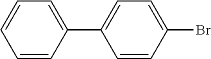 [SMILES: Brc1ccc(-c2ccccc2)cc1] | 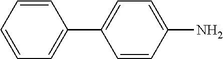 [SMILES: Nc1ccc(-c2ccccc2)cc1] | 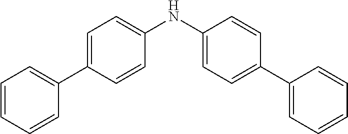 [SMILES: c1ccc(-c2ccc(Nc3ccc(-c4ccccc4)cc3)cc2)cc1] | 25.0 g (87%) | 321.15 |
| SUB 1-2 | 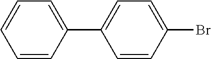 [SMILES: Brc1ccc(-c2ccccc2)cc1] | 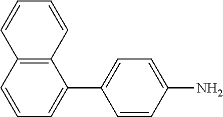 [SMILES: Nc1ccc(-c2cccc3ccccc23)cc1] | 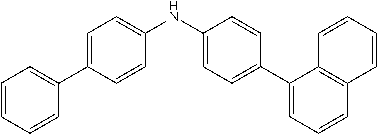 [SMILES: c1ccc(-c2ccc(Nc3ccc(-c4cccc5ccccc45)cc3)cc2)cc1] | 25.8 g (78%) | 371.17 |
| SUB 1-3 | 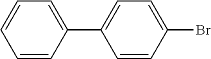 [SMILES: Brc1ccc(-c2ccccc2)cc1] | 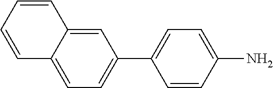 [SMILES: Nc1ccc(-c2ccc3ccccc3c2)cc1] | 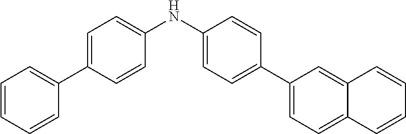 [SMILES: c1ccc(-c2ccc(Nc3ccc(-c4ccc5ccccc5c4)cc3)cc2)cc1] | 30.6 g (86%) | 371.17 |
| SUB 1-4 | 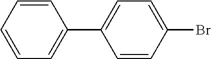 [SMILES: Brc1ccc(-c2ccccc2)cc1] | 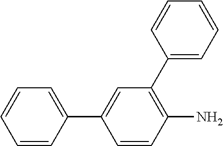 [SMILES: Nc1ccc(-c2ccccc2)cc1-c1ccccc1] | 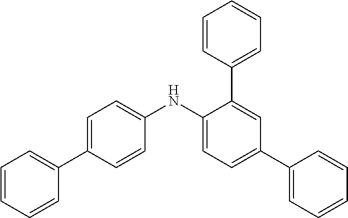 [SMILES: c1ccc(-c2ccc(Nc3ccc(-c4ccccc4)cc3-c3ccccc3)cc2)cc1] | 25.6 g (84%) | 397.18 |
| SUB 1-5 | 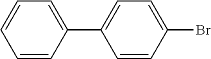 [SMILES: Brc1ccc(-c2ccccc2)cc1] | 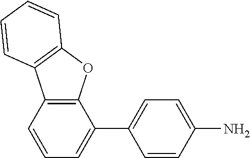 [SMILES: Nc1ccc(-c2cccc3c2oc2ccccc23)cc1] | 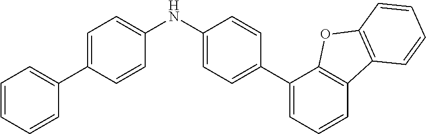 [SMILES: c1ccc(-c2ccc(Nc3ccc(-c4cccc5c4oc4ccccc45)cc3)cc2)cc1] | 27.3 g (74%) | 411.16 |
| SUB 1-6 | 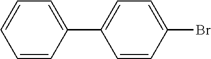 [SMILES: Brc1ccc(-c2ccccc2)cc1] | 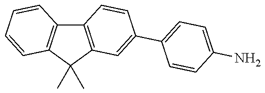 [SMILES: CC1(C)c2ccccc2-c2ccc(-c3ccc(N)cc3)cc21] | 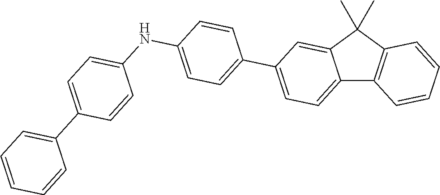 [SMILES: CC1(C)c2ccccc2-c2ccc(-c3ccc(Nc4ccc(-c5ccccc5)cc4)cc3)cc21] | 30.6 g (78%) | 437.21 |
| SUB 1-7 | 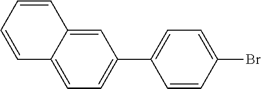 [SMILES: Brc1ccc(-c2ccc3ccccc3c2)cc1] | 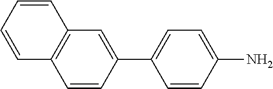 [SMILES: Nc1ccc(-c2ccc3ccccc3c2)cc1] | 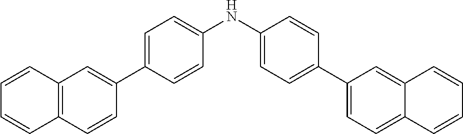 [SMILES: c1ccc2cc(-c3ccc(Nc4ccc(-c5ccc6ccccc6c5)cc4)cc3)ccc2c1] | 24.2 g (81%) | 421.18 |
| SUB 1-8 | 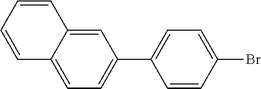 [SMILES: Brc1ccc(-c2ccc3ccccc3c2)cc1] | 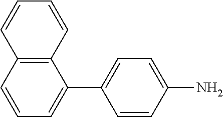 [SMILES: Nc1ccc(-c2cccc3ccccc23)cc1] | 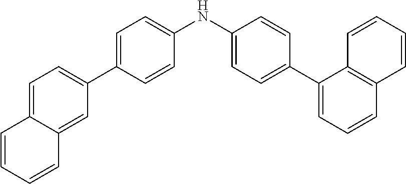 [SMILES: c1ccc2cc(-c3ccc(Nc4ccc(-c5cccc6ccccc56)cc4)cc3)ccc2c1] | 31.7 g (84%) | 421.18 |
| SUB 1-9 | 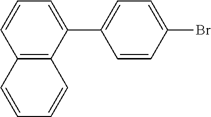 [SMILES: Brc1ccc(-c2cccc3ccccc23)cc1] | 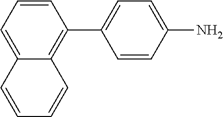 [SMILES: Nc1ccc(-c2cccc3ccccc23)cc1] | 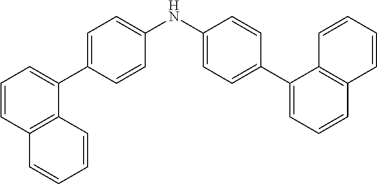 [SMILES: c1ccc2c(-c3ccc(Nc4ccc(-c5cccc6ccccc56)cc4)cc3)cccc2c1] | 31.0 g (81%) | 421.18 |
| SUB 1-10 | 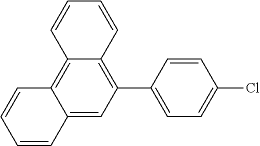 [SMILES: Clc1ccc(-c2cc3ccccc3c3ccccc23)cc1] | 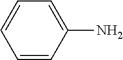 | 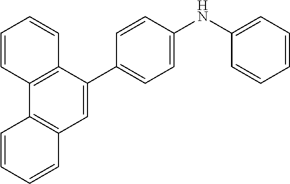 [SMILES: c1ccc(Nc2ccc(-c3cc4ccccc4c4ccccc34)cc2)cc1] | 20.7 g (87%) | 345.15 |
| SUB 1-11 | 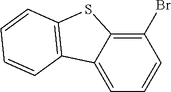 [SMILES: Brc1cccc2c1sc1ccccc12] | 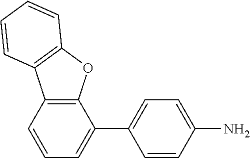 [SMILES: Nc1ccc(-c2cccc3c2oc2ccccc23)cc1] | 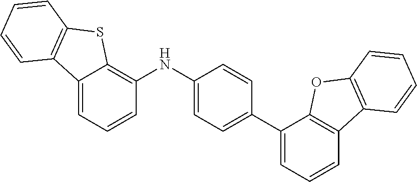 [SMILES: c1ccc2c(c1)oc1c(-c3ccc(Nc4cccc5c4sc4ccccc45)cc3)cccc12] | 31.6 g (80%) | 441.12 |
| SUB 1-12 | 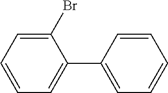 [SMILES: Brc1ccccc1-c1ccccc1] | 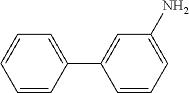 [SMILES: Nc1cccc(-c2ccccc2)c1] | 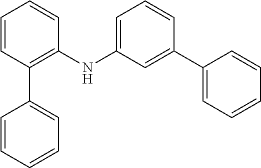 [SMILES: c1ccc(-c2cccc(Nc3ccccc3-c3ccccc3)c2)cc1] | 21.9 g (76%) | 321.15 |
| SUB 1-13 | 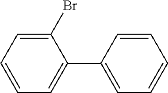 [SMILES: Brc1ccccc1-c1ccccc1] | 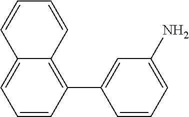 [SMILES: Nc1cccc(-c2cccc3ccccc23)c1] | 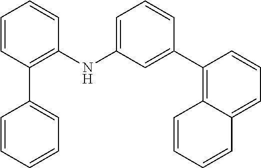 [SMILES: c1ccc(-c2ccccc2Nc2cccc(-c3cccc4ccccc34)c2)cc1] | 270.0 g (81%) | 371.17 |
| SUB 1-14 | 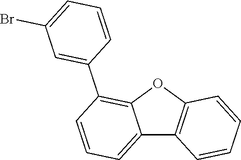 [SMILES: Brc1cccc(-c2cccc3c2oc2ccccc23)c1] | 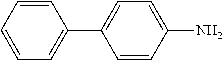 [SMILES: Nc1ccc(-c2ccccc2)cc1] | 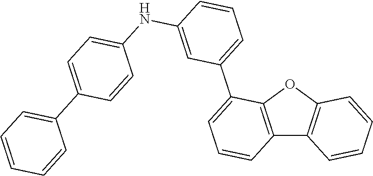 [SMILES: c1ccc(-c2ccc(Nc3cccc(-c4cccc5c4oc4ccccc45)c3)cc2)cc1] | 29.1 g (79%) | 411.16 |
| SUB 1-15 | 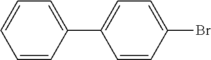 [SMILES: Brc1ccc(-c2ccccc2)cc1] | 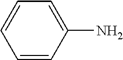 | 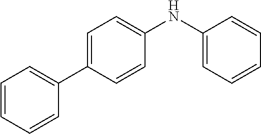 [SMILES: c1ccc(Nc2ccc(-c3ccccc3)cc2)cc1] | 18.7 g (85%) | 245.12 |
| SUB 1-16 | 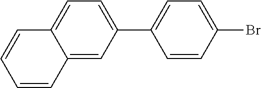 [SMILES: Brc1ccc(-c2ccc3ccccc3c2)cc1] | 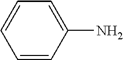 | 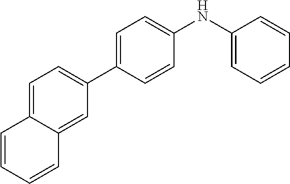 [SMILES: c1ccc(Nc2ccc(-c3ccc4ccccc4c3)cc2)cc1] | 20.1 g (76%) | 295.14 |
| SUB 1-17 | 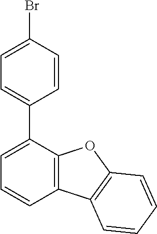 [SMILES: Brc1ccc(-c2cccc3c2oc2ccccc23)cc1] | 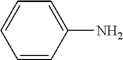 | 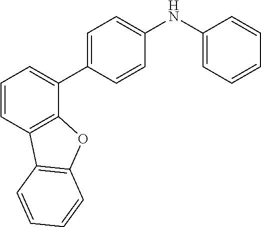 [SMILES: c1ccc(Nc2ccc(-c3cccc4c3oc3ccccc34)cc2)cc1] | 23.4 g (78%) | 335.13 |
| SUB 1-18 | 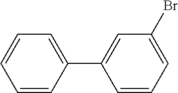 [SMILES: Brc1cccc(-c2ccccc2)c1] | 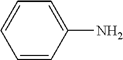 | 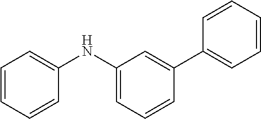 [SMILES: c1ccc(Nc2cccc(-c3ccccc3)c2)cc1] | 15.6 g (71%) | 245.12 |
| SUB 1-19 | 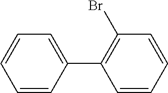 [SMILES: Brc1ccccc1-c1ccccc1] | 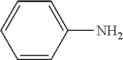 | 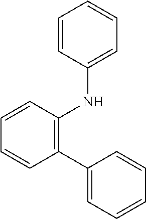 [SMILES: c1ccc(Nc2ccccc2-c2ccccc2)cc1] | 16.7 g (76%) | 245.12 |
| SUB 1-20 | 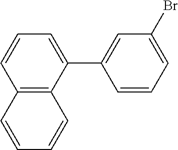 [SMILES: Brc1cccc(-c2cccc3ccccc23)c1] | 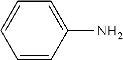 | 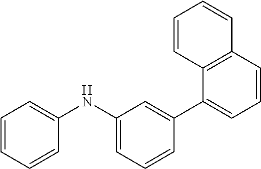 [SMILES: c1ccc(Nc2cccc(-c3cccc4ccccc34)c2)cc1] | 20.9 g (79%) | 295.14 |
| SUB 1-21 | 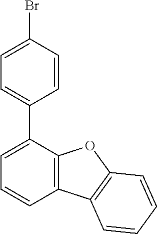 [SMILES: Brc1ccc(-c2cccc3c2oc2ccccc23)cc1] | 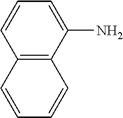 [SMILES: Nc1cccc2ccccc12] | 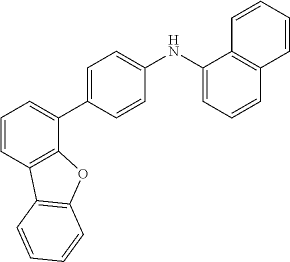 [SMILES: c1ccc2c(Nc3ccc(-c4cccc5c4oc4ccccc45)cc3)cccc2c1] | 28.7 g (83%) | 385.15 |
| SUB 1-22 | 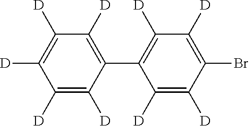 [SMILES: [2H]c1c([2H])c([2H])c(-c2c([2H])c([2H])c(Br)c([2H])c2[2H])c([2H])c1[2H]] | 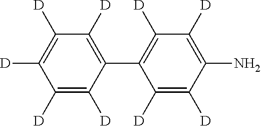 [SMILES: [2H]c1c([2H])c([2H])c(-c2c([2H])c([2H])c(N)c([2H])c2[2H])c([2H])c1[2H]] | 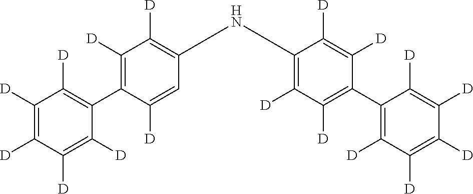 [SMILES: [2H]c1cc(Nc2c([2H])c([2H])c(-c3c([2H])c([2H])c([2H])c([2H])c3[2H])c([2H])c2[2H])c([2H])c([2H])c1-c1c([2H])c([2H])c([2H])c([2H])c1[2H]] | 25.8 g (85%) | 339.26 |
| SUB 1-23 | 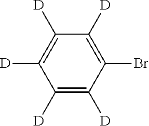 [SMILES: [2H]c1c([2H])c([2H])c(Br)c([2H])c1[2H]] | 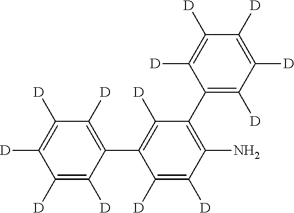 [SMILES: [2H]c1c([2H])c([2H])c(-c2c([2H])c([2H])c(N)c(-c3c([2H])c([2H])c([2H])c([2H])c3[2H])c2[2H])c([2H])c1[2H]] | 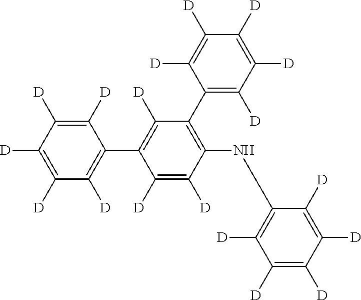 [SMILES: [2H]c1c([2H])c([2H])c(Nc2c([2H])c([2H])c(-c3c([2H])c([2H])c([2H])c([2H])c3[2H])c([2H])c2-c2c([2H])c([2H])c([2H])c([2H])c2[2H])c([2H])c1[2H]] | 24.6 g (81%) | 339.26 |
| SUB 1-24 | 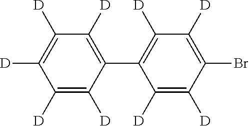 [SMILES: [2H]c1c([2H])c([2H])c(-c2c([2H])c([2H])c(Br)c([2H])c2[2H])c([2H])c1[2H]] | 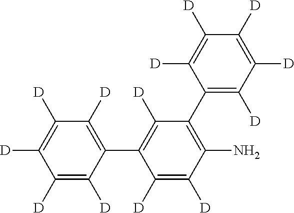 [SMILES: [2H]c1c([2H])c([2H])c(-c2c([2H])c([2H])c(N)c(-c3c([2H])c([2H])c([2H])c([2H])c3[2H])c2[2H])c([2H])c1[2H]] | 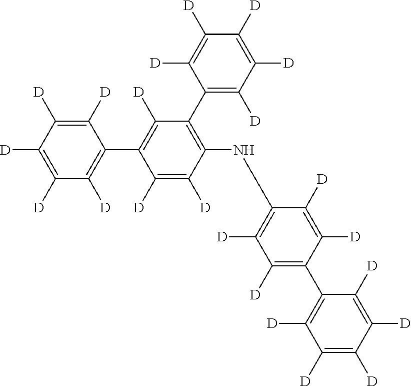 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3-c3c([2H])c([2H])c([2H])c([2H])c3[2H])c([2H])c2[2H])c([2H])c1[2H]] | 30.1 g (80%) | 419.32 |
| SUB 1-25 | 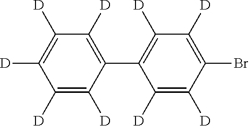 [SMILES: [2H]c1c([2H])c([2H])c(-c2c([2H])c([2H])c(Br)c([2H])c2[2H])c([2H])c1[2H]] | 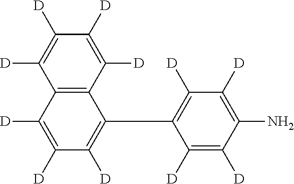 [SMILES: [2H]c1c([2H])c(-c2c([2H])c([2H])c([2H])c3c([2H])c([2H])c([2H])c([2H])c23)c([2H])c([2H])c1N] | 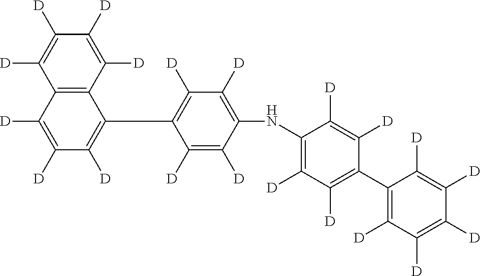 [SMILES: [2H]c1c([2H])c([2H])c(-c2c([2H])c([2H])c(Nc3c([2H])c([2H])c(-c4c([2H])c([2H])c([2H])c5c([2H])c([2H])c([2H])c([2H])c45)c([2H])c3[2H])c([2H])c2[2H])c([2H])c1[2H]] | 27.7 g (79%) | 391.29 |
| SUB 1-26 | 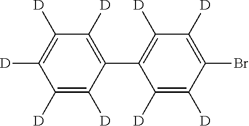 [SMILES: [2H]c1c([2H])c([2H])c(-c2c([2H])c([2H])c(Br)c([2H])c2[2H])c([2H])c1[2H]] | 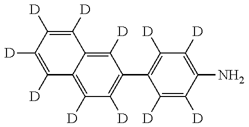 [SMILES: [2H]c1c([2H])c(-c2c([2H])c([2H])c3c([2H])c([2H])c([2H])c([2H])c3c2[2H])c([2H])c([2H])c1N] | 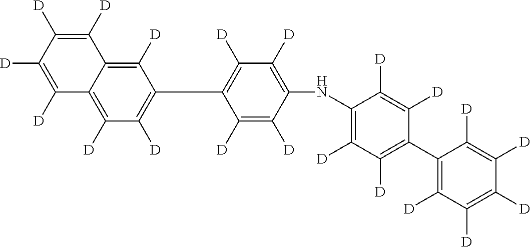 [SMILES: [2H]c1c([2H])c([2H])c(-c2c([2H])c([2H])c(Nc3c([2H])c([2H])c(-c4c([2H])c([2H])c5c([2H])c([2H])c([2H])c([2H])c5c4[2H])c([2H])c3[2H])c([2H])c2[2H])c([2H])c1[2H]] | 27.0 g (77%) | 391.29 |
| SUB 1-27 | 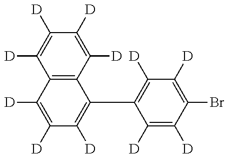 [SMILES: [2H]c1c([2H])c(-c2c([2H])c([2H])c([2H])c3c([2H])c([2H])c([2H])c([2H])c23)c([2H])c([2H])c1Br] | 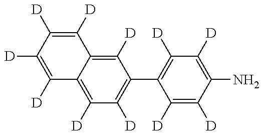 [SMILES: [2H]c1c([2H])c(-c2c([2H])c([2H])c3c([2H])c([2H])c([2H])c([2H])c3c2[2H])c([2H])c([2H])c1N] | 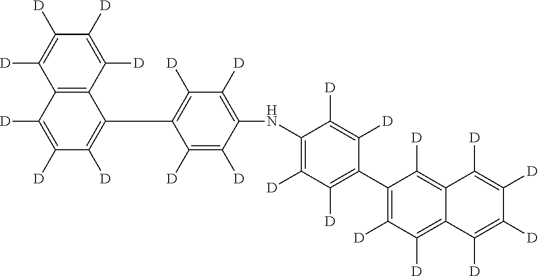 [SMILES: [2H]c1c([2H])c(-c2c([2H])c([2H])c3c([2H])c([2H])c([2H])c([2H])c3c2[2H])c([2H])c([2H])c1Nc1c([2H])c([2H])c(-c2c([2H])c([2H])c([2H])c3c([2H])c([2H])c([2H])c([2H])c23)c([2H])c1[2H]] | 30.2 g (76%) | 443.32 |
| SUB 1-28 | 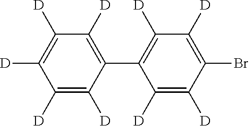 [SMILES: [2H]c1c([2H])c([2H])c(-c2c([2H])c([2H])c(Br)c([2H])c2[2H])c([2H])c1[2H]] | 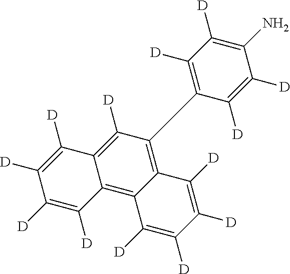 [SMILES: [2H]c1c([2H])c(-c2c([2H])c3c([2H])c([2H])c([2H])c([2H])c3c3c([2H])c([2H])c([2H])c([2H])c23)c([2H])c([2H])c1N] | 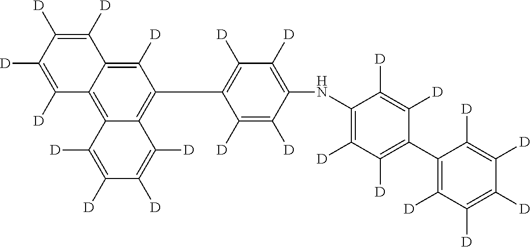 [SMILES: [2H]c1c([2H])c([2H])c(-c2c([2H])c([2H])c(Nc3c([2H])c([2H])c(-c4c([2H])c5c([2H])c([2H])c([2H])c([2H])c5c5c([2H])c([2H])c([2H])c([2H])c45)c([2H])c3[2H])c([2H])c2[2H])c([2H])c1[2H]] | 33.0 g (83%) | 443.32 |
| SUB 1-29 | 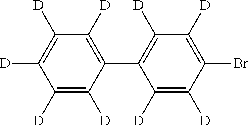 [SMILES: [2H]c1c([2H])c([2H])c(-c2c([2H])c([2H])c(Br)c([2H])c2[2H])c([2H])c1[2H]] | 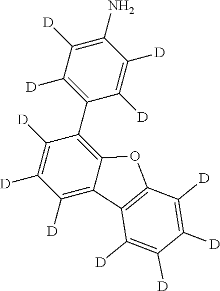 [SMILES: [2H]c1c([2H])c(-c2c([2H])c([2H])c([2H])c3c2oc2c([2H])c([2H])c([2H])c([2H])c23)c([2H])c([2H])c1N] | 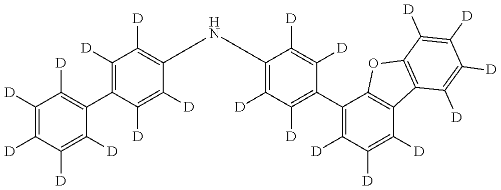 [SMILES: [2H]c1c([2H])c([2H])c(-c2c([2H])c([2H])c(Nc3c([2H])c([2H])c(-c4c([2H])c([2H])c([2H])c5c4oc4c([2H])c([2H])c([2H])c([2H])c45)c([2H])c3[2H])c([2H])c2[2H])c([2H])c1[2H]] | 31.7 g (82%) | 431.29 |
| SUB 1-30 | 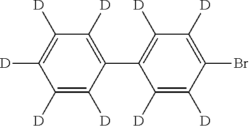 [SMILES: [2H]c1c([2H])c([2H])c(-c2c([2H])c([2H])c(Br)c([2H])c2[2H])c([2H])c1[2H]] | 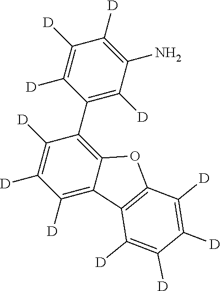 [SMILES: [2H]c1c([2H])c(N)c([2H])c(-c2c([2H])c([2H])c([2H])c3c2oc2c([2H])c([2H])c([2H])c([2H])c23)c1[2H]] | 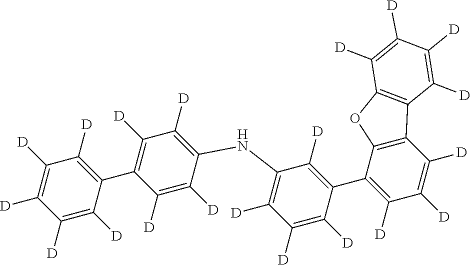 [SMILES: [2H]c1c([2H])c([2H])c(-c2c([2H])c([2H])c(Nc3c([2H])c([2H])c([2H])c(-c4c([2H])c([2H])c([2H])c5c4oc4c([2H])c([2H])c([2H])c([2H])c45)c3[2H])c([2H])c2[2H])c([2H])c1[2H]] | 30.2 g (78%) | 431.29 |
| SUB 1-31 | 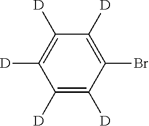 [SMILES: [2H]c1c([2H])c([2H])c(Br)c([2H])c1[2H]] | 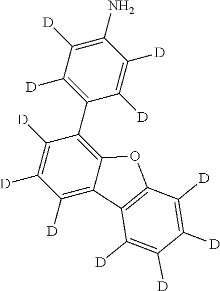 [SMILES: [2H]c1c([2H])c(-c2c([2H])c([2H])c([2H])c3c2oc2c([2H])c([2H])c([2H])c([2H])c23)c([2H])c([2H])c1N] | 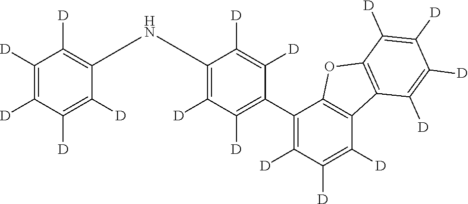 [SMILES: [2H]c1c([2H])c([2H])c(Nc2c([2H])c([2H])c(-c3c([2H])c([2H])c([2H])c4c3oc3c([2H])c([2H])c([2H])c([2H])c34)c([2H])c2[2H])c([2H])c1[2H]] | 26.8 g (85%) | 351.23 |
| SUB 1-32 | 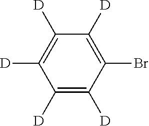 [SMILES: [2H]c1c([2H])c([2H])c(Br)c([2H])c1[2H]] | 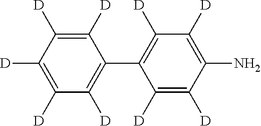 [SMILES: [2H]c1c([2H])c([2H])c(-c2c([2H])c([2H])c(N)c([2H])c2[2H])c([2H])c1[2H]] | 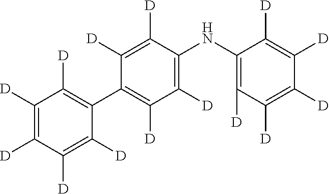 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] | 19.9 g (86%) | 259.21 |
| SUB 1-33 | 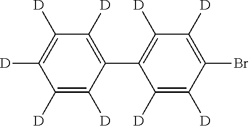 [SMILES: [2H]c1c([2H])c([2H])c(-c2c([2H])c([2H])c(Br)c([2H])c2[2H])c([2H])c1[2H]] | 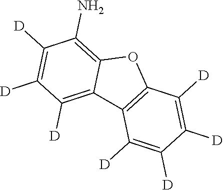 [SMILES: [2H]c1c([2H])c([2H])c2c(oc3c(N)c([2H])c([2H])c([2H])c32)c1[2H]] | 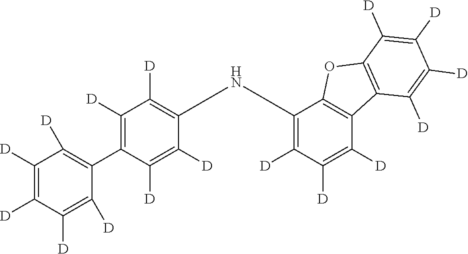 [SMILES: [2H]c1c([2H])c([2H])c(-c2c([2H])c([2H])c(Nc3c([2H])c([2H])c([2H])c4c3oc3c([2H])c([2H])c([2H])c([2H])c34)c([2H])c2[2H])c([2H])c1[2H]] | 25.1 g (80%) | 351.23 |
| SUB 1-34 | 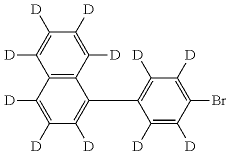 [SMILES: [2H]c1c([2H])c(-c2c([2H])c([2H])c([2H])c3c([2H])c([2H])c([2H])c([2H])c23)c([2H])c([2H])c1Br] | 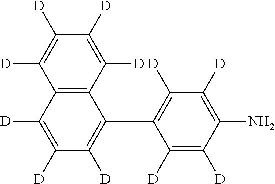 [SMILES: [2H]c1c([2H])c(-c2c([2H])c([2H])c([2H])c3c([2H])c([2H])c([2H])c([2H])c23)c([2H])c([2H])c1N] | 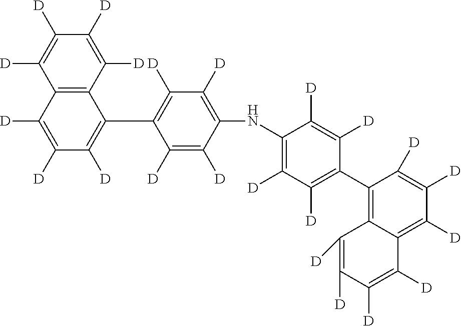 [SMILES: [2H]c1c([2H])c(-c2c([2H])c([2H])c([2H])c3c([2H])c([2H])c([2H])c([2H])c23)c([2H])c([2H])c1Nc1c([2H])c([2H])c(-c2c([2H])c([2H])c([2H])c3c([2H])c([2H])c([2H])c([2H])c23)c([2H])c1[2H]] | 28.2 g (71%) | 443.32 |

### 2. Synthesis of SUB 2

SUB 2 may be synthesized by Reaction Formula 2 below, but is not limited thereto (X may be O or S, Hal¹ may be Br, I, or C1, and Hal² may be Br).

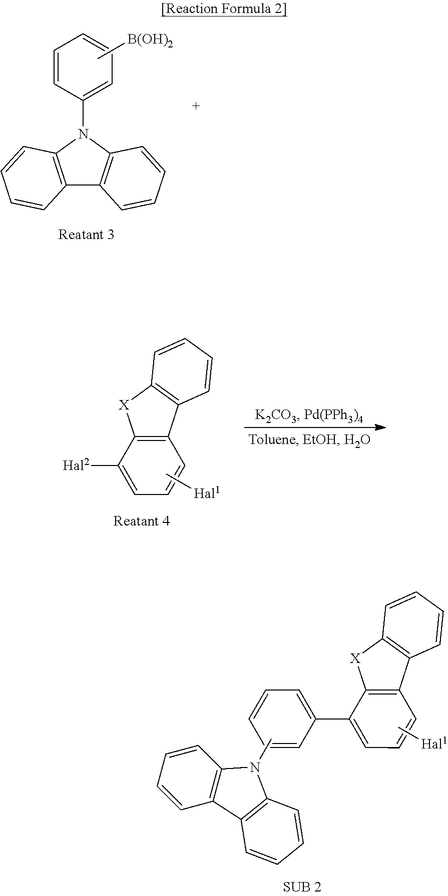

Reactant 3 (174.1 mmol), Reactant 4 (182.8 mmol), K₂CO₃ (348.3 mmol), and Pd(PPh₃)₄ (3.48 mmol) were added to a 3000 mL flask under nitrogen air flow, and after toluene, ethanol, and water were added, stirred, and refluxed. After the reaction was finished, an organic layer was extracted by using toluene and water. The extracted solution was treated with MgSO₄ to remove remaining moisture, concentrated under reduced pressure, purified by using a column chromatography method, and then recrystallized to obtain SUB 2.

In addition, Reactants 3 and 4 substituted with deuterium may be synthesized in the same manner as Reaction Formula 2. The synthesized result of SUB 2 is expressed by Table 2 below.

TABLE 2

| Item | Reactant 3 | Reactant 4 | SUB 2 |
|---|---|---|---|
| SUB 2-1 | 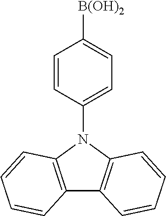 [SMILES: OB(O)c1ccc(-n2c3ccccc3c3ccccc32)cc1] | 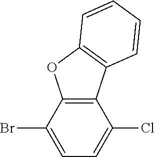 [SMILES: Clc1ccc(Br)c2oc3ccccc3c12] | 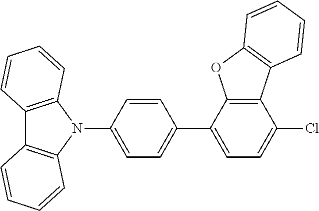 [SMILES: Clc1ccc(-c2ccc(-n3c4ccccc4c4ccccc43)cc2)c2oc3ccccc3c12] |
| Sub 202 | | 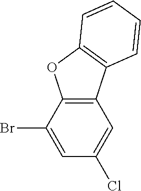 [SMILES: Clc1cc(Br)c2oc3ccccc3c2c1] | 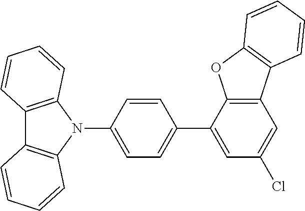 [SMILES: Clc1cc(-c2ccc(-n3c4ccccc4c4ccccc43)cc2)c2oc3ccccc3c2c1] |
| SUB 2-3 | | 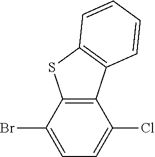 [SMILES: Clc1ccc(Br)c2sc3ccccc3c12] | 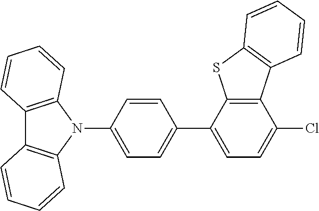 [SMILES: Clc1ccc(-c2ccc(-n3c4ccccc4c4ccccc43)cc2)c2sc3ccccc3c12] |
| SUB 2-4 | | 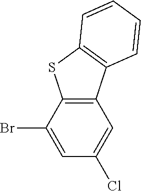 [SMILES: Clc1cc(Br)c2sc3ccccc3c2c1] | 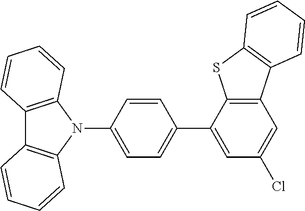 [SMILES: Clc1cc(-c2ccc(-n3c4ccccc4c4ccccc43)cc2)c2sc3ccccc3c2c1] |
| SUB 2-5 | 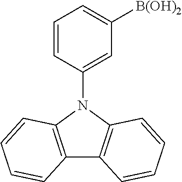 [SMILES: OB(O)c1cccc(-n2c3ccccc3c3ccccc32)c1] | 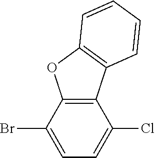 [SMILES: Clc1ccc(Br)c2oc3ccccc3c12] | 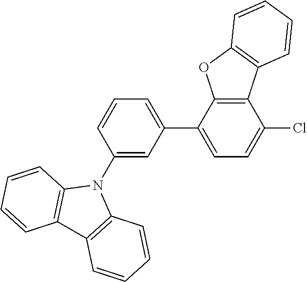 [SMILES: Clc1ccc(-c2cccc(-n3c4ccccc4c4ccccc43)c2)c2oc3ccccc3c12] |
| SUB 2-6 | | 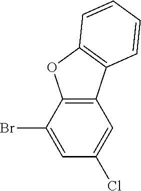 [SMILES: Clc1cc(Br)c2oc3ccccc3c2c1] | 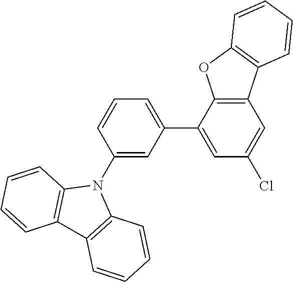 [SMILES: Clc1cc(-c2cccc(-n3c4ccccc4c4ccccc43)c2)c2oc3ccccc3c2c1] |
| SUB 2-7 | | 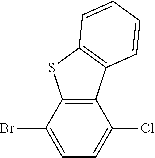 [SMILES: Clc1ccc(Br)c2sc3ccccc3c12] | 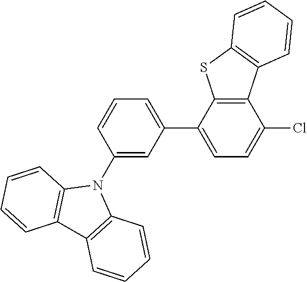 [SMILES: Clc1ccc(-c2cccc(-n3c4ccccc4c4ccccc43)c2)c2sc3ccccc3c12] |
| SUB 2-8 | | 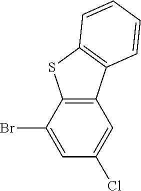 [SMILES: Clc1cc(Br)c2sc3ccccc3c2c1] | 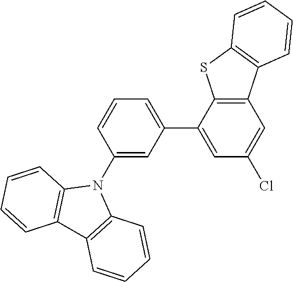 [SMILES: Clc1cc(-c2cccc(-n3c4ccccc4c4ccccc43)c2)c2sc3ccccc3c2c1] |
| SUB 2-9 | 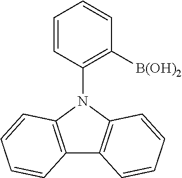 [SMILES: OB(O)c1ccccc1-n1c2ccccc2c2ccccc21] | 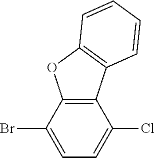 [SMILES: Clc1ccc(Br)c2oc3ccccc3c12] | 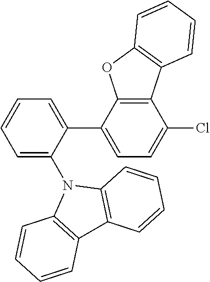 [SMILES: Clc1ccc(-c2ccccc2-n2c3ccccc3c3ccccc32)c2oc3ccccc3c12] |
| SUB 2-10 | | 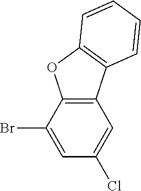 [SMILES: Clc1cc(Br)c2oc3ccccc3c2c1] | 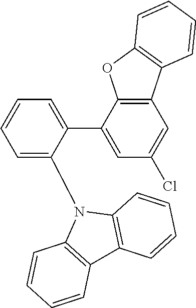 [SMILES: Clc1cc(-c2ccccc2-n2c3ccccc3c3ccccc32)c2oc3ccccc3c2c1] |
| SUB 2-11 | | 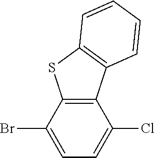 [SMILES: Clc1ccc(Br)c2sc3ccccc3c12] | 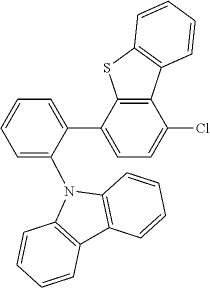 [SMILES: Clc1ccc(-c2ccccc2-n2c3ccccc3c3ccccc32)c2sc3ccccc3c12] |
| SUB 2-12 | | 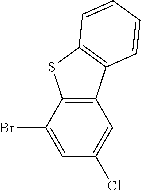 [SMILES: Clc1cc(Br)c2sc3ccccc3c2c1] | 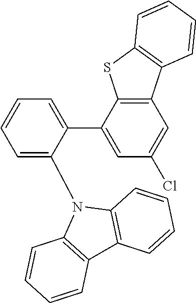 [SMILES: Clc1cc(-c2ccccc2-n2c3ccccc3c3ccccc32)c2sc3ccccc3c2c1] |
| SUB 2-13 | 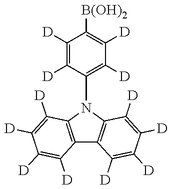 [SMILES: [2H]c1c([2H])c(-n2c3c([2H])c([2H])c([2H])c([2H])c3c3c([2H])c([2H])c([2H])c([2H])c32)c([2H])c([2H])c1B(O)O] | 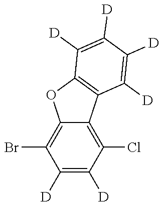 [SMILES: [2H]c1c([2H])c([2H])c2c(oc3c(Br)c([2H])c([2H])c(Cl)c32)c1[2H]] | 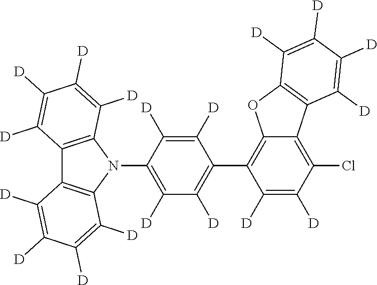 [SMILES: [2H]c1c([2H])c(-n2c3c([2H])c([2H])c([2H])c([2H])c3c3c([2H])c([2H])c([2H])c([2H])c32)c([2H])c([2H])c1-c1c([2H])c([2H])c(Cl)c2c1oc1c([2H])c([2H])c([2H])c([2H])c12] |
| SUB 2-14 | | 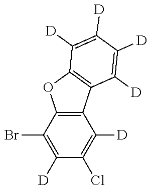 [SMILES: [2H]c1c([2H])c([2H])c2c(oc3c(Br)c([2H])c(Cl)c([2H])c32)c1[2H]] | 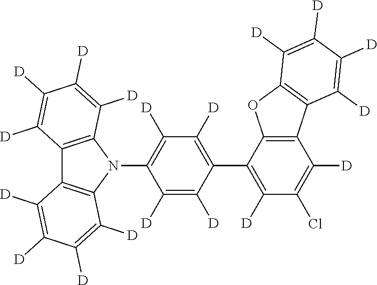 [SMILES: [2H]c1c([2H])c(-n2c3c([2H])c([2H])c([2H])c([2H])c3c3c([2H])c([2H])c([2H])c([2H])c32)c([2H])c([2H])c1-c1c([2H])c(Cl)c([2H])c2c1oc1c([2H])c([2H])c([2H])c([2H])c12] |
| SUB 2-15 | | 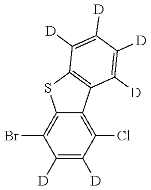 [SMILES: [2H]c1c([2H])c([2H])c2c(sc3c(Br)c([2H])c([2H])c(Cl)c32)c1[2H]] | 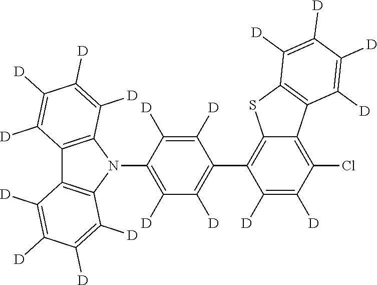 [SMILES: [2H]c1c([2H])c(-n2c3c([2H])c([2H])c([2H])c([2H])c3c3c([2H])c([2H])c([2H])c([2H])c32)c([2H])c([2H])c1-c1c([2H])c([2H])c(Cl)c2c1sc1c([2H])c([2H])c([2H])c([2H])c12] |
| SUB 2-16 | | 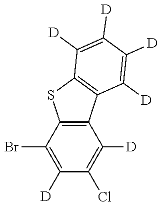 [SMILES: [2H]c1c([2H])c([2H])c2c(sc3c(Br)c([2H])c(Cl)c([2H])c32)c1[2H]] | 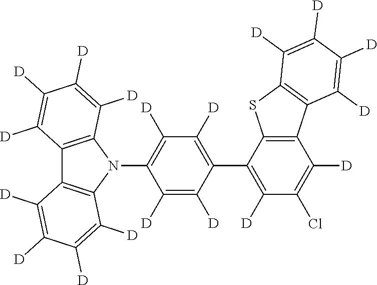 [SMILES: [2H]c1c([2H])c(-n2c3c([2H])c([2H])c([2H])c([2H])c3c3c([2H])c([2H])c([2H])c([2H])c32)c([2H])c([2H])c1-c1c([2H])c(Cl)c([2H])c2c1sc1c([2H])c([2H])c([2H])c([2H])c12] |
| SUB 2-17 | 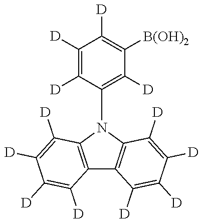 [SMILES: [2H]c1c([2H])c(B(O)O)c([2H])c(-n2c3c([2H])c([2H])c([2H])c([2H])c3c3c([2H])c([2H])c([2H])c([2H])c32)c1[2H]] | 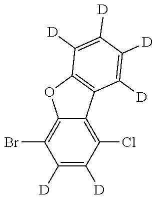 [SMILES: [2H]c1c([2H])c([2H])c2c(oc3c(Br)c([2H])c([2H])c(Cl)c32)c1[2H]] | 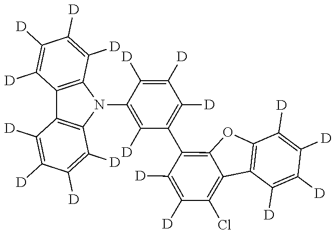 [SMILES: [2H]c1c([2H])c(-c2c([2H])c([2H])c(Cl)c3c2oc2c([2H])c([2H])c([2H])c([2H])c23)c([2H])c(-n2c3c([2H])c([2H])c([2H])c([2H])c3c3c([2H])c([2H])c([2H])c([2H])c32)c1[2H]] |
| SUB 2-18 | | 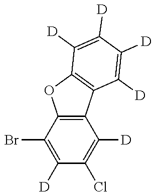 [SMILES: [2H]c1c([2H])c([2H])c2c(oc3c(Br)c([2H])c(Cl)c([2H])c32)c1[2H]] | 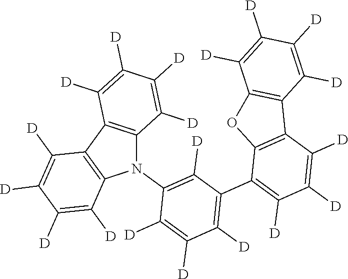 [SMILES: [2H]c1c([2H])c(-c2c([2H])c([2H])c([2H])c3c2oc2c([2H])c([2H])c([2H])c([2H])c23)c([2H])c(-n2c3c([2H])c([2H])c([2H])c([2H])c3c3c([2H])c([2H])c([2H])c([2H])c32)c1[2H]] |
| SUB 2-19 | | 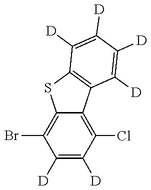 [SMILES: [2H]c1c([2H])c([2H])c2c(sc3c(Br)c([2H])c([2H])c(Cl)c32)c1[2H]] | 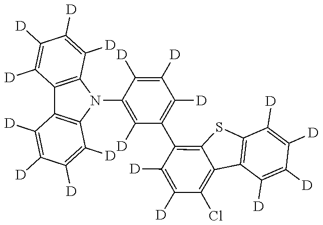 [SMILES: [2H]c1c([2H])c(-c2c([2H])c([2H])c(Cl)c3c2sc2c([2H])c([2H])c([2H])c([2H])c23)c([2H])c(-n2c3c([2H])c([2H])c([2H])c([2H])c3c3c([2H])c([2H])c([2H])c([2H])c32)c1[2H]] |
| SUB 2-20 | | 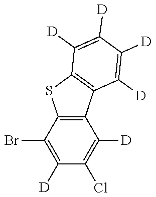 [SMILES: [2H]c1c([2H])c([2H])c2c(sc3c(Br)c([2H])c(Cl)c([2H])c32)c1[2H]] | 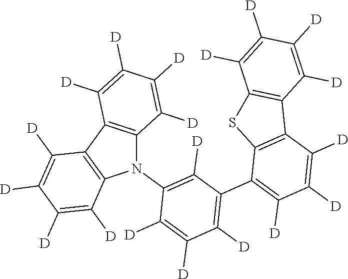 [SMILES: [2H]c1c([2H])c(-c2c([2H])c([2H])c([2H])c3c2sc2c([2H])c([2H])c([2H])c([2H])c23)c([2H])c(-n2c3c([2H])c([2H])c([2H])c([2H])c3c3c([2H])c([2H])c([2H])c([2H])c32)c1[2H]] |
| SUB 2-21 | 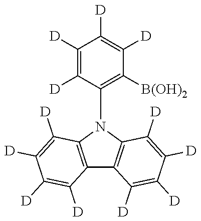 [SMILES: [2H]c1c([2H])c([2H])c(-n2c3c([2H])c([2H])c([2H])c([2H])c3c3c([2H])c([2H])c([2H])c([2H])c32)c(B(O)O)c1[2H]] | 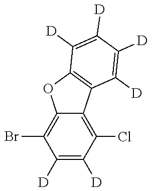 [SMILES: [2H]c1c([2H])c([2H])c2c(oc3c(Br)c([2H])c([2H])c(Cl)c32)c1[2H]] | 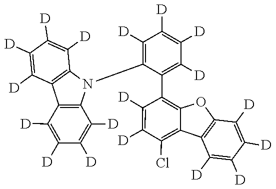 [SMILES: [2H]c1c([2H])c([2H])c(-n2c3c([2H])c([2H])c([2H])c([2H])c3c3c([2H])c([2H])c([2H])c([2H])c32)c(-c2c([2H])c([2H])c(Cl)c3c2oc2c([2H])c([2H])c([2H])c([2H])c23)c1[2H]] |
| SUB 2-22 | | 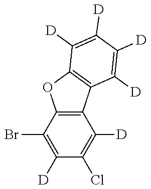 [SMILES: [2H]c1c([2H])c([2H])c2c(oc3c(Br)c([2H])c(Cl)c([2H])c32)c1[2H]] | 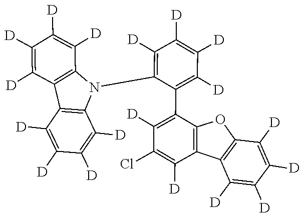 [SMILES: [2H]c1c([2H])c([2H])c(-n2c3c([2H])c([2H])c([2H])c([2H])c3c3c([2H])c([2H])c([2H])c([2H])c32)c(-c2c([2H])c(Cl)c([2H])c3c2oc2c([2H])c([2H])c([2H])c([2H])c23)c1[2H]] |
| SUB 2-23 | | 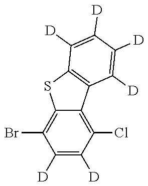 [SMILES: [2H]c1c([2H])c([2H])c2c(sc3c(Br)c([2H])c([2H])c(Cl)c32)c1[2H]] | 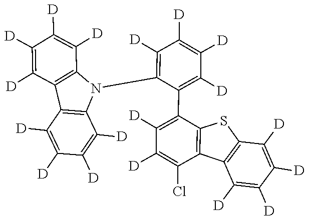 [SMILES: [2H]c1c([2H])c([2H])c(-n2c3c([2H])c([2H])c([2H])c([2H])c3c3c([2H])c([2H])c([2H])c([2H])c32)c(-c2c([2H])c([2H])c(Cl)c3c2sc2c([2H])c([2H])c([2H])c([2H])c23)c1[2H]] |
| SUB 2-24 | | 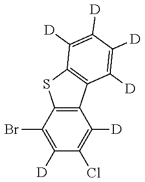 [SMILES: [2H]c1c([2H])c([2H])c2c(sc3c(Br)c([2H])c(Cl)c([2H])c32)c1[2H]] | 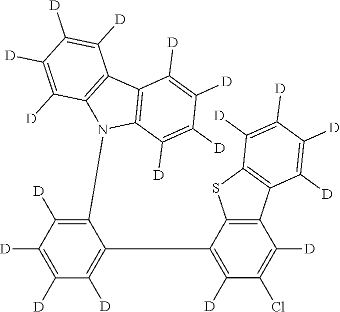 [SMILES: [2H]c1c([2H])c([2H])c(-n2c3c([2H])c([2H])c([2H])c([2H])c3c3c([2H])c([2H])c([2H])c([2H])c32)c(-c2c([2H])c(Cl)c([2H])c3c2sc2c([2H])c([2H])c([2H])c([2H])c23)c1[2H]] |
| SUB 2-25 | 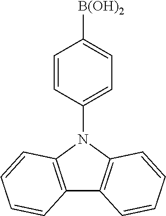 [SMILES: OB(O)c1ccc(-n2c3ccccc3c3ccccc32)cc1] | 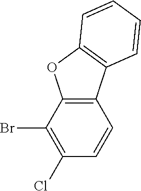 [SMILES: Clc1ccc2c(oc3ccccc32)c1Br] | 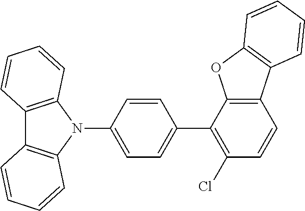 [SMILES: Clc1ccc2c(oc3ccccc32)c1-c1ccc(-n2c3ccccc3c3ccccc32)cc1] |
| SUB 2-26 | | 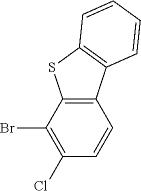 [SMILES: Clc1ccc2c(sc3ccccc32)c1Br] | 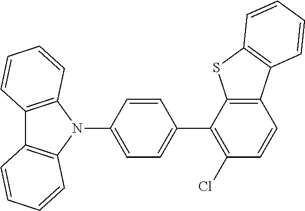 [SMILES: Clc1ccc2c(sc3ccccc32)c1-c1ccc(-n2c3ccccc3c3ccccc32)cc1] |
| SUB 2-27 | 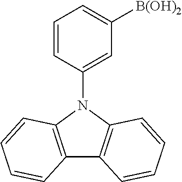 [SMILES: OB(O)c1cccc(-n2c3ccccc3c3ccccc32)c1] | 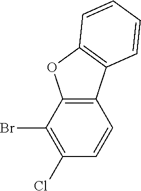 [SMILES: Clc1ccc2c(oc3ccccc32)c1Br] | 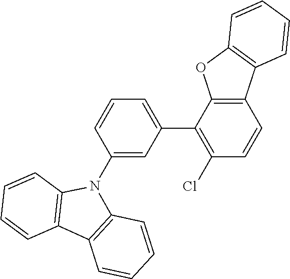 [SMILES: Clc1ccc2c(oc3ccccc32)c1-c1cccc(-n2c3ccccc3c3ccccc32)c1] |
| SUB 2-28 | | 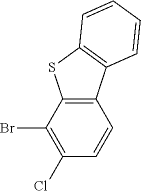 [SMILES: Clc1ccc2c(sc3ccccc32)c1Br] | 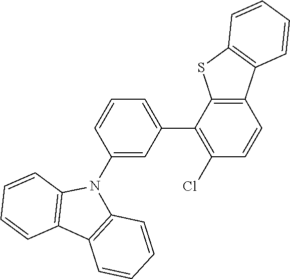 [SMILES: Clc1ccc2c(sc3ccccc32)c1-c1cccc(-n2c3ccccc3c3ccccc32)c1] |
| SUB 2-29 | 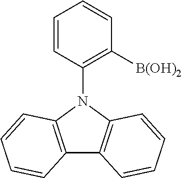 [SMILES: OB(O)c1ccccc1-n1c2ccccc2c2ccccc21] | 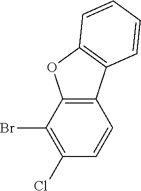 [SMILES: Clc1ccc2c(oc3ccccc32)c1Br] | 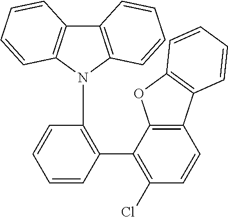 [SMILES: Clc1ccc2c(oc3ccccc32)c1-c1ccccc1-n1c2ccccc2c2ccccc21] |
| SUB 2-30 | | 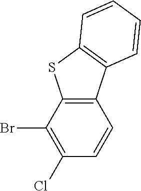 [SMILES: Clc1ccc2c(sc3ccccc32)c1Br] | 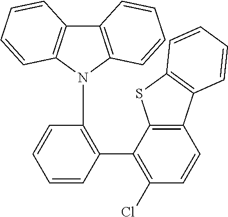 [SMILES: Clc1ccc2c(sc3ccccc32)c1-c1ccccc1-n1c2ccccc2c2ccccc21] |
| SUB 2-31 | 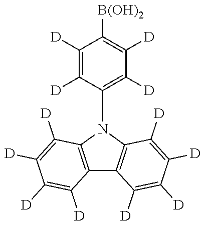 [SMILES: [2H]c1c([2H])c(-n2c3c([2H])c([2H])c([2H])c([2H])c3c3c([2H])c([2H])c([2H])c([2H])c32)c([2H])c([2H])c1B(O)O] | 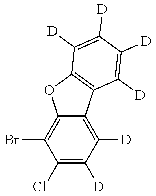 [SMILES: [2H]c1c([2H])c([2H])c2c(oc3c(Br)c(Cl)c([2H])c([2H])c32)c1[2H]] | 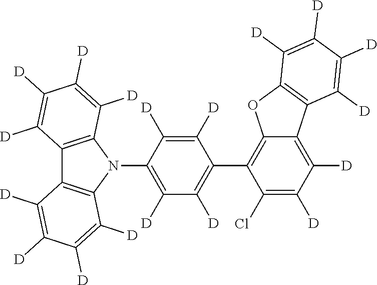 [SMILES: [2H]c1c([2H])c(-n2c3c([2H])c([2H])c([2H])c([2H])c3c3c([2H])c([2H])c([2H])c([2H])c32)c([2H])c([2H])c1-c1c(Cl)c([2H])c([2H])c2c1oc1c([2H])c([2H])c([2H])c([2H])c12] |
| SUB 2-32 | | 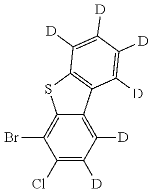 [SMILES: [2H]c1c([2H])c([2H])c2c(sc3c(Br)c(Cl)c([2H])c([2H])c32)c1[2H]] | 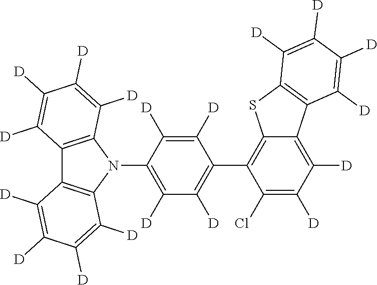 [SMILES: [2H]c1c([2H])c(-n2c3c([2H])c([2H])c([2H])c([2H])c3c3c([2H])c([2H])c([2H])c([2H])c32)c([2H])c([2H])c1-c1c(Cl)c([2H])c([2H])c2c1sc1c([2H])c([2H])c([2H])c([2H])c12] |
| SUB 2-33 | 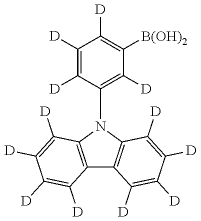 [SMILES: [2H]c1c([2H])c(B(O)O)c([2H])c(-n2c3c([2H])c([2H])c([2H])c([2H])c3c3c([2H])c([2H])c([2H])c([2H])c32)c1[2H]] | 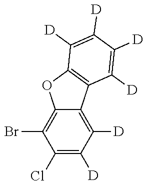 [SMILES: [2H]c1c([2H])c([2H])c2c(oc3c(Br)c(Cl)c([2H])c([2H])c32)c1[2H]] | 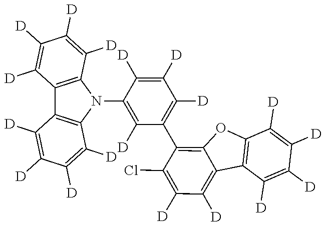 [SMILES: [2H]c1c([2H])c(-c2c(Cl)c([2H])c([2H])c3c2oc2c([2H])c([2H])c([2H])c([2H])c23)c([2H])c(-n2c3c([2H])c([2H])c([2H])c([2H])c3c3c([2H])c([2H])c([2H])c([2H])c32)c1[2H]] |
| SUB 2-34 | | 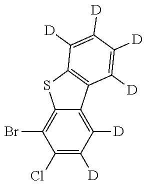 [SMILES: [2H]c1c([2H])c([2H])c2c(sc3c(Br)c(Cl)c([2H])c([2H])c32)c1[2H]] | 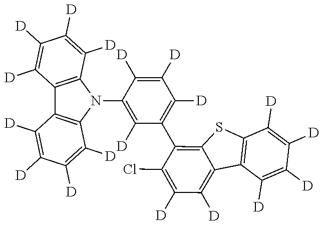 [SMILES: [2H]c1c([2H])c(-c2c(Cl)c([2H])c([2H])c3c2sc2c([2H])c([2H])c([2H])c([2H])c23)c([2H])c(-n2c3c([2H])c([2H])c([2H])c([2H])c3c3c([2H])c([2H])c([2H])c([2H])c32)c1[2H]] |
| SUB 2-35 | 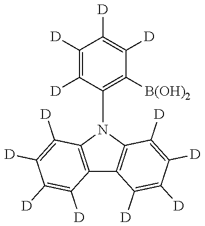 [SMILES: [2H]c1c([2H])c([2H])c(-n2c3c([2H])c([2H])c([2H])c([2H])c3c3c([2H])c([2H])c([2H])c([2H])c32)c(B(O)O)c1[2H]] | 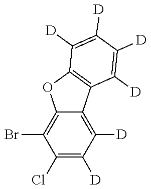 [SMILES: [2H]c1c([2H])c([2H])c2c(oc3c(Br)c(Cl)c([2H])c([2H])c32)c1[2H]] | 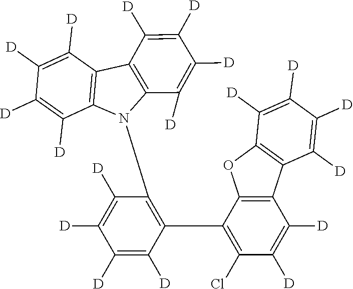 [SMILES: [2H]c1c([2H])c([2H])c(-n2c3c([2H])c([2H])c([2H])c([2H])c3c3c([2H])c([2H])c([2H])c([2H])c32)c(-c2c(Cl)c([2H])c([2H])c3c2oc2c([2H])c([2H])c([2H])c([2H])c23)c1[2H]] |
| SUB 2-36 | | 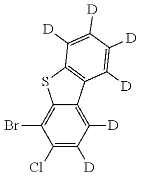 [SMILES: [2H]c1c([2H])c([2H])c2c(sc3c(Br)c(Cl)c([2H])c([2H])c32)c1[2H]] | 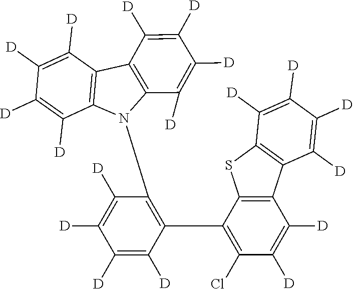 [SMILES: [2H]c1c([2H])c([2H])c(-n2c3c([2H])c([2H])c([2H])c([2H])c3c3c([2H])c([2H])c([2H])c([2H])c32)c(-c2c(Cl)c([2H])c([2H])c3c2sc2c([2H])c([2H])c([2H])c([2H])c23)c1[2H]] |

| Item | Obtained amount (Yield) | [M + H]⁺ |
|---|---|---|
| SUB 2-1 | 69.8 g (86%) | 443.11 |
| SUB 2-2 | 66.5 g (82%) | 443.11 |
| SUB 2-3 | 60.7 g (76%) | 459.08 |
| SUB 2-4 | 55.9 g (70%) | 459.08 |
| SUB 2-5 | 62.6 g (81%) | 443.11 |
| SUB 2-6 | 57.9 g (72%) | 443.11 |
| SUB 2-7 | 64.7 g (81%) | 459.08 |
| SUB 2-8 | 54.3 g (68%) | 459.08 |
| SUB 2-9 | 58.7 g (76%) | 443.11 |
| SUB 2-10 | 54.8 g (68%) | 443.11 |
| SUB 2-11 | 66.3 g (83%) | 459.08 |
| SUB 2-12 | 51.1 g (64%) | 459.08 |
| SUB 2-13 | 35.1 g (85%) | 461.22 |
| SUB 2-14 | 36.0 g (87%) | 461.22 |
| SUB 2-15 | 32.5 g (76%) | 477.20 |
| SUB 2-16 | 31.7 g (74%) | 477.20 |
| SUB 2-17 | 34.7 g (84%) | 461.22 |
| SUB 2-18 | 34.3 g (83%) | 461.22 |
| SUB 2-19 | 30.4 g (71%) | 477.20 |
| SUB 2-20 | 30.8 g (72%) | 477.20 |
| SUB 2-21 | 35.6 g (86%) | 461.22 |
| SUB 2-22 | 36.0 g (87%) | 461.22 |
| SUB 2-23 | 34.2 g (80%) | 477.20 |
| SUB 2-24 | 32.1 g (75%) | 477.20 |
| SUB 2-25 | 30.6 g (77%) | 443.11 |
| SUB 2-26 | 29.2 g (71%) | 459.08 |
| SUB 2-27 | 29.8 g (75%) | 443.11 |
| SUB 2-28 | 28.0 g (68%) | 459.08 |
| SUB 2-29 | 29.0 g (73%) | 443.11 |
| SUB 2-30 | 28.8 g (70%) | 459.08 |
| SUB 2-31 | 29.4 g (71%) | 461.22 |
| SUB 2-32 | 29.9 g (70%) | 477.20 |
| SUB 2-33 | 31.0 g (75%) | 461.22 |
| SUB 2-34 | 30.8 g (72%) | 477.20 |
| SUB 2-35 | 29.4 g (71%) | 461.22 |
| SUB 2-36 | 30.8 g (72%) | 477.20 |

### 3. Synthesis of Product

A compound (product) may be synthesized as follows, but is not limited thereto (X may be O or S, and Hal¹ may be Br, I, or C1).

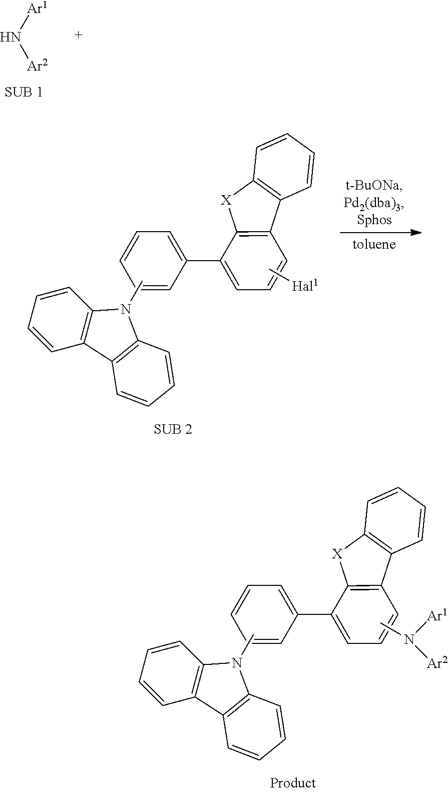

SUB 1 (16.33 mmol), SUB 2 (15.56 mmol), t-BuONa (31.11 mmol), Pd₂(dba)₃ (0.31 mmol), sphos (0.622 mmol), and toluene were added, stirred, and refluxed in a 500 mL flask under nitrogen air flow. After the reaction was finished, an organic layer was extracted by using toluene and water. The extracted solution was treated with MgSO₄ to remove remaining moisture, concentrated under reduced pressure, purified by using a column chromatography method, and then recrystallized to obtain the compound. The synthesized result of the compound (product) is expressed by Tables 3 to 20 below.

TABLE 3

| | | | | Ob- | |
|---|---|---|---|---|---|
| | | | | tained | |
| Com- | SUB 2-1 | | | amount | [M + |
| pound | SUB 2-13 | SUB 1 | Product | (Yield) | H]⁺ |
| 1 | 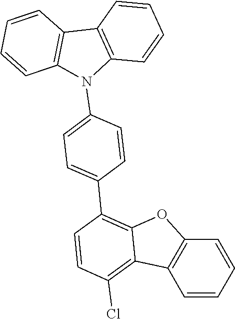 [SMILES: Clc1ccc(-c2ccc(-n3c4ccccc4c4ccccc43)cc2)c2oc3ccccc3c12] | 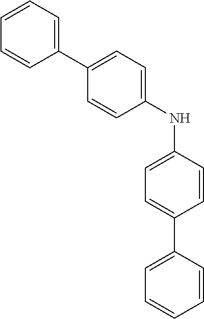 [SMILES: c1ccc(-c2ccc(Nc3ccc(-c4ccccc4)cc3)cc2)cc1] | 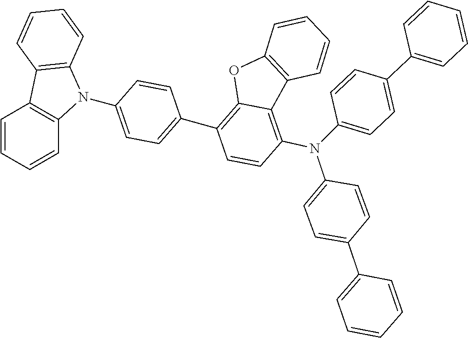 [SMILES: c1ccc(-c2ccc(N(c3ccc(-c4ccccc4)cc3)c3ccc(-c4ccc(-n5c6ccccc6c6ccccc65)cc4)c4oc5ccccc5c34)cc2)cc1] | 9.3 g (82%) | 728.28 |
| 2 | | 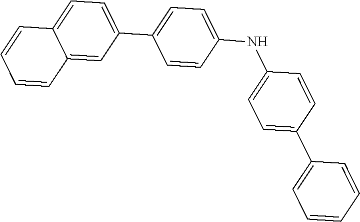 [SMILES: c1ccc(-c2ccc(Nc3ccc(-c4ccc5ccccc5c4)cc3)cc2)cc1] | 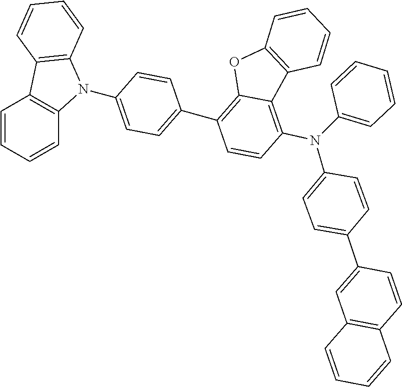 [SMILES: c1ccc(N(c2ccc(-c3ccc4ccccc4c3)cc2)c2ccc(-c3ccc(-n4c5ccccc5c5ccccc54)cc3)c3oc4ccccc4c23)cc1] | 10.3 g (85%) | 778.30 |
| 3 | | 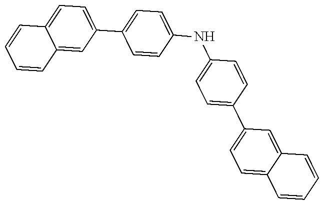 [SMILES: c1ccc2cc(-c3ccc(Nc4ccc(-c5ccc6ccccc6c5)cc4)cc3)ccc2c1] | 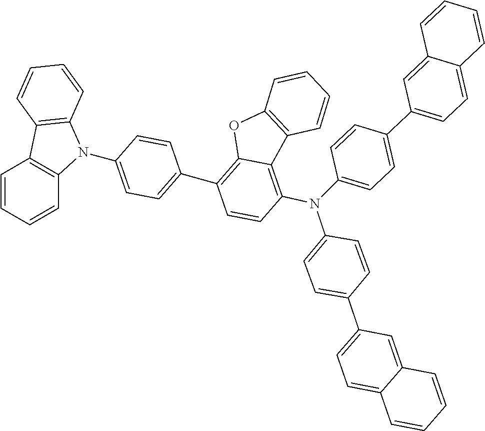 [SMILES: c1ccc2cc(-c3ccc(N(c4ccc(-c5ccc6ccccc6c5)cc4)c4ccc(-c5ccc(-n6c7ccccc7c7ccccc76)cc5)c5oc6ccccc6c45)cc3)ccc2c1] | 9.3 g (72%) | 828.31 |
| 4 | | 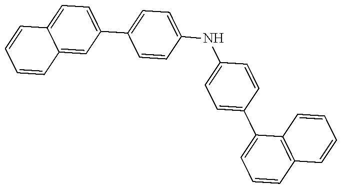 [SMILES: c1ccc2cc(-c3ccc(Nc4ccc(-c5cccc6ccccc56)cc4)cc3)ccc2c1] | 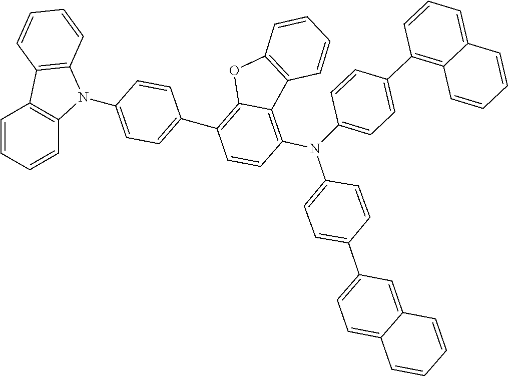 [SMILES: c1ccc2cc(-c3ccc(N(c4ccc(-c5cccc6ccccc56)cc4)c4ccc(-c5ccc(-n6c7ccccc7c7ccccc76)cc5)c5oc6ccccc6c45)cc3)ccc2c1] | 10.7 g (83%) | 828.31 |
| 5 | | 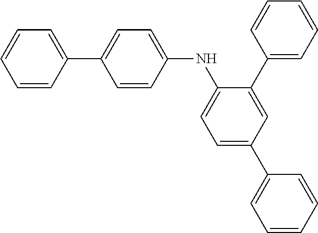 [SMILES: c1ccc(-c2ccc(Nc3ccc(-c4ccccc4)cc3-c3ccccc3)cc2)cc1] | 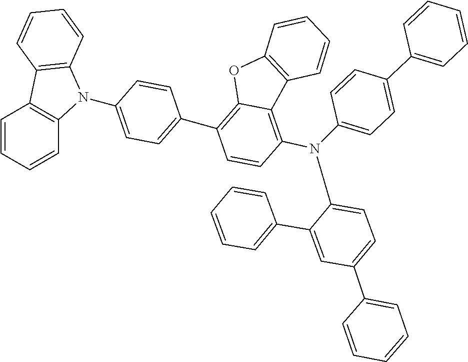 [SMILES: c1ccc(-c2ccc(N(c3ccc(-c4ccccc4)cc3-c3ccccc3)c3ccc(-c4ccc(-n5c6ccccc6c6ccccc65)cc4)c4oc5ccccc5c34)cc2)cc1] | 9.5 g (76%) | 804.31 |
| 6 | | 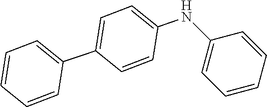 [SMILES: c1ccc(Nc2ccc(-c3ccccc3)cc2)cc1] | 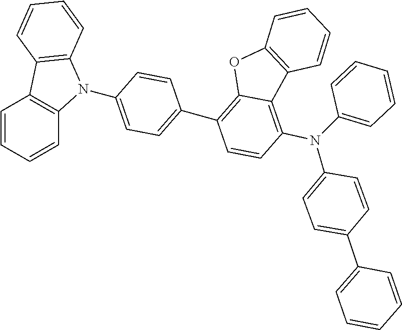 [SMILES: c1ccc(-c2ccc(N(c3ccccc3)c3ccc(-c4ccc(-n5c6ccccc6c6ccccc65)cc4)c4oc5ccccc5c34)cc2)cc1] | 8.6 g (85%) | 625.25 |
| 7 | | 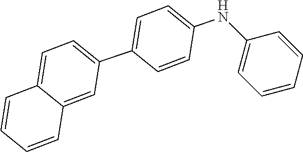 [SMILES: c1ccc(Nc2ccc(-c3ccc4ccccc4c3)cc2)cc1] | 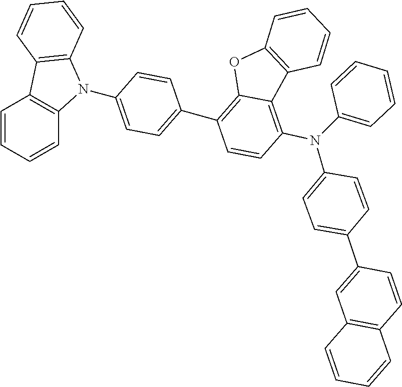 [SMILES: c1ccc(N(c2ccc(-c3ccc4ccccc4c3)cc2)c2ccc(-c3ccc(-n4c5ccccc5c5ccccc54)cc3)c3oc4ccccc4c23)cc1] | 9.4 g (86%) | 702.27 |
| 8 | | 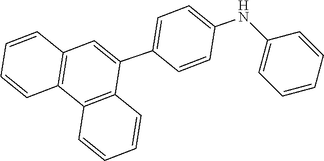 [SMILES: c1ccc(Nc2ccc(-c3cc4ccccc4c4ccccc34)cc2)cc1] | 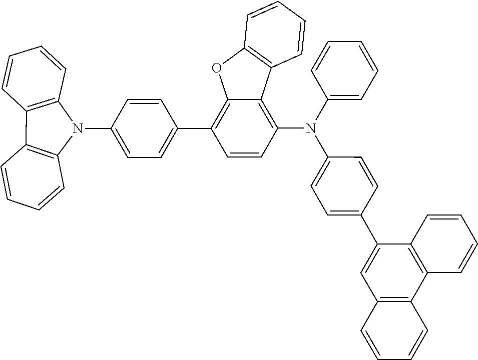 [SMILES: c1ccc(N(c2ccc(-c3cc4ccccc4c4ccccc34)cc2)c2ccc(-c3ccc(-n4c5ccccc5c5ccccc54)cc3)c3oc4ccccc4c23)cc1] | 8.3 g (71%) | 752.28 |
| 9 | | 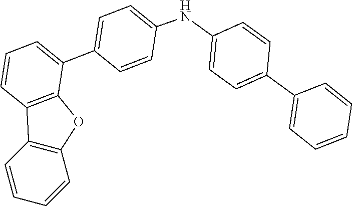 [SMILES: c1ccc(-c2ccc(Nc3ccc(-c4cccc5c4oc4ccccc45)cc3)cc2)cc1] | 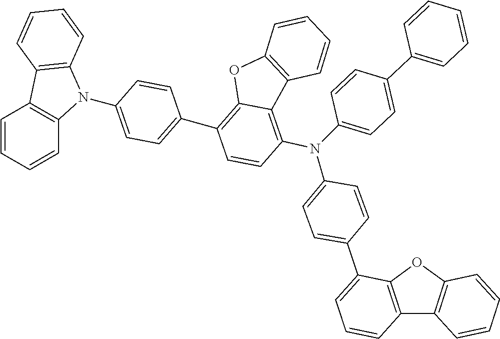 [SMILES: c1ccc(-c2ccc(N(c3ccc(-c4cccc5c4oc4ccccc45)cc3)c3ccc(-c4ccc(-n5c6ccccc6c6ccccc65)cc4)c4oc5ccccc5c34)cc2)cc1] | 9.7 g (76%) | 818.29 |
| 10 | | 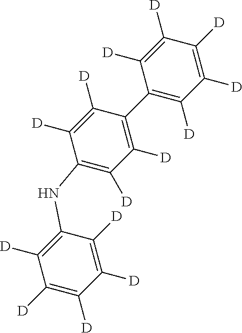 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] | 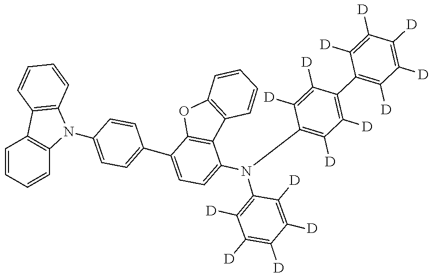 [SMILES: [2H]c1c([2H])c([2H])c(-c2c([2H])c([2H])c(N(c3c([2H])c([2H])c([2H])c([2H])c3[2H])c3ccc(-c4ccc(-n5c6ccccc6c6ccccc65)cc4)c4oc5ccccc5c34)c([2H])c2[2H])c([2H])c1[2H]] | 9.0 g (87%) | 666.34 |
| 11 | | 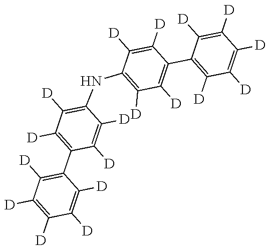 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] | 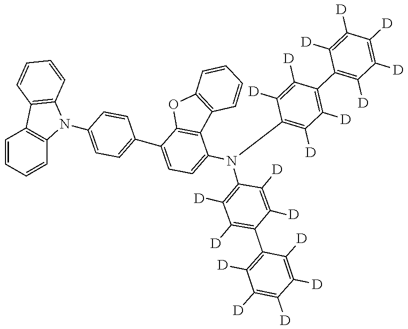 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3ccc(-c4ccc(-n5c6ccccc6c6ccccc65)cc4)c4oc5ccccc5c34)c([2H])c2[2H])c([2H])c1[2H]] | 9.5 g (82%) | 746.40 |
| 12 | | 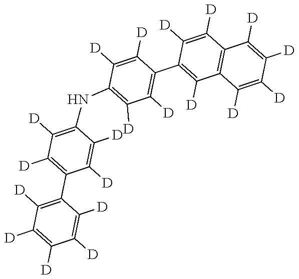 [SMILES: [2H]c1c([2H])c([2H])c(-c2c([2H])c([2H])c(Nc3c([2H])c([2H])c(-c4c([2H])c([2H])c5c([2H])c([2H])c([2H])c([2H])c5c4[2H])c([2H])c3[2H])c([2H])c2[2H])c([2H])c1[2H]] | 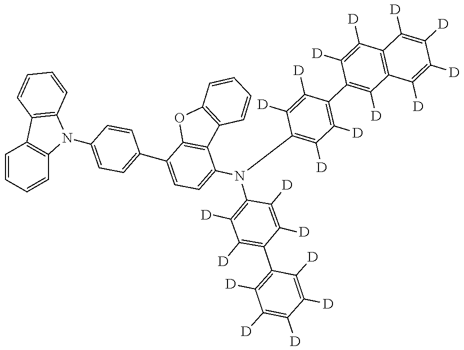 [SMILES: [2H]c1c([2H])c([2H])c(-c2c([2H])c([2H])c(N(c3c([2H])c([2H])c(-c4c([2H])c([2H])c5c([2H])c([2H])c([2H])c([2H])c5c4[2H])c([2H])c3[2H])c3ccc(-c4ccc(-n5c6ccccc6c6ccccc65)cc4)c4oc5ccccc5c34)c([2H])c2[2H])c([2H])c1[2H]] | 9.6 g (77%) | 798.42 |
| 13 | 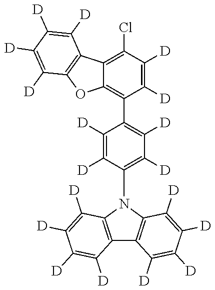 [SMILES: [2H]c1c([2H])c(-n2c3c([2H])c([2H])c([2H])c([2H])c3c3c([2H])c([2H])c([2H])c([2H])c32)c([2H])c([2H])c1-c1c([2H])c([2H])c(Cl)c2c1oc1c([2H])c([2H])c([2H])c([2H])c12] | 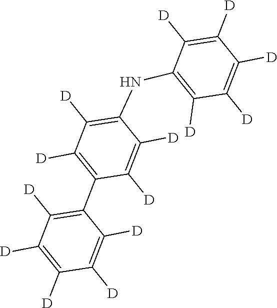 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] | 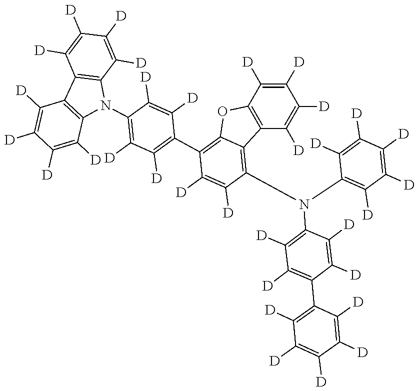 [SMILES: [2H]c1c([2H])c([2H])c(-c2c([2H])c([2H])c(N(c3c([2H])c([2H])c([2H])c([2H])c3[2H])c3c([2H])c([2H])c(-c4c([2H])c([2H])c(-n5c6c([2H])c([2H])c([2H])c([2H])c6c6c([2H])c([2H])c([2H])c([2H])c65)c([2H])c4[2H])c4oc5c([2H])c([2H])c([2H])c([2H])c5c34)c([2H])c2[2H])c([2H])c1[2H]] | 8.4 g (79%) | 684.45 |
| 14 | | 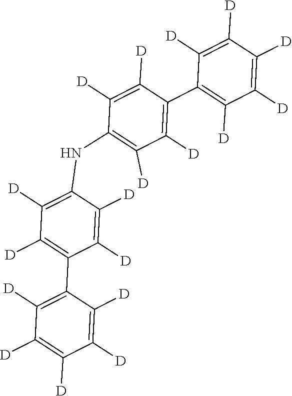 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] | 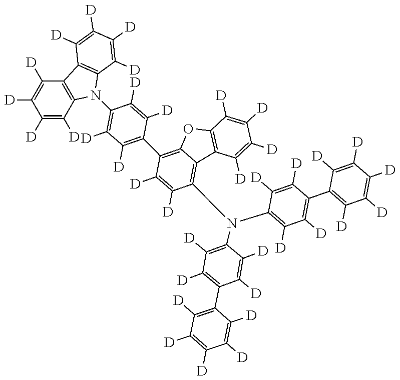 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c(-c4c([2H])c([2H])c(-n5c6c([2H])c([2H])c([2H])c([2H])c6c6c([2H])c([2H])c([2H])c([2H])c65)c([2H])c4[2H])c4oc5c([2H])c([2H])c([2H])c([2H])c5c34)c([2H])c2[2H])c([2H])c1[2H]] | 9.5 g (80%) | 764.51 |
| 15 | | 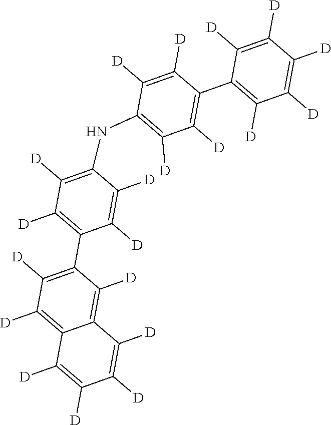 [SMILES: [2H]c1c([2H])c([2H])c(-c2c([2H])c([2H])c(Nc3c([2H])c([2H])c(-c4c([2H])c([2H])c5c([2H])c([2H])c([2H])c([2H])c5c4[2H])c([2H])c3[2H])c([2H])c2[2H])c([2H])c1[2H]] | 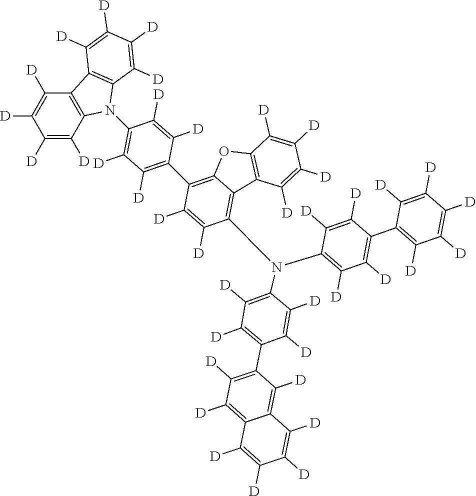 [SMILES: [2H]c1c([2H])c([2H])c(-c2c([2H])c([2H])c(N(c3c([2H])c([2H])c(-c4c([2H])c([2H])c5c([2H])c([2H])c([2H])c([2H])c5c4[2H])c([2H])c3[2H])c3c([2H])c([2H])c(-c4c([2H])c([2H])c(-n5c6c([2H])c([2H])c([2H])c([2H])c6c6c([2H])c([2H])c([2H])c([2H])c65)c([2H])c4[2H])c4oc5c([2H])c([2H])c([2H])c([2H])c5c34)c([2H])c2[2H])c([2H])c1[2H]] | 9.5 g (75%) | 816.54 |

TABLE 4

| | | | | Ob- | |
|---|---|---|---|---|---|
| | | | | tained | |
| Com- | SUB 2-2 | | | amount | [M + |
| pound | SUB 2-14 | SUB 1 | Product | (Yield) | H]⁺ |
| 16 | 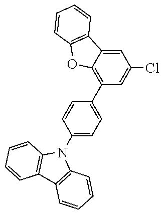 [SMILES: Clc1cc(-c2ccc(-n3c4ccccc4c4ccccc43)cc2)c2oc3ccccc3c2c1] | 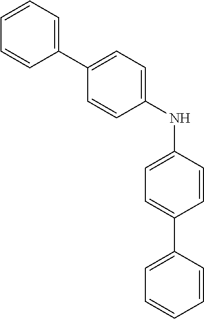 [SMILES: c1ccc(-c2ccc(Nc3ccc(-c4ccccc4)cc3)cc2)cc1] | 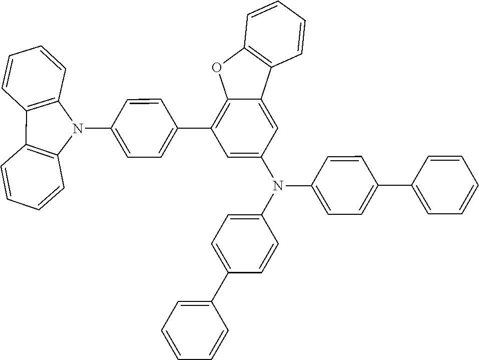 [SMILES: c1ccc(-c2ccc(N(c3ccc(-c4ccccc4)cc3)c3cc(-c4ccc(-n5c6ccccc6c6ccccc65)cc4)c4oc5ccccc5c4c3)cc2)cc1] | 8.6 g (76%) | 728.28 |
| 17 | | 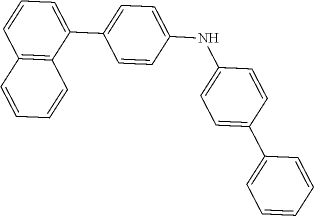 [SMILES: c1ccc(-c2ccc(Nc3ccc(-c4cccc5ccccc45)cc3)cc2)cc1] | 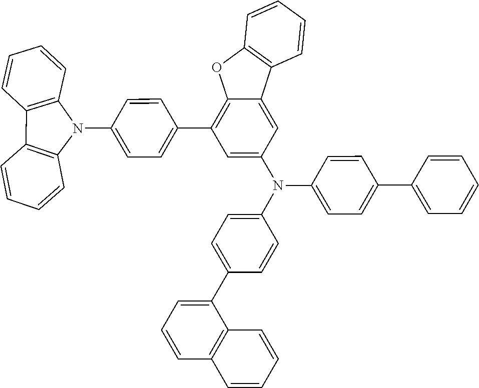 [SMILES: c1ccc(-c2ccc(N(c3ccc(-c4cccc5ccccc45)cc3)c3cc(-c4ccc(-n5c6ccccc6c6ccccc65)cc4)c4oc5ccccc5c4c3)cc2)cc1] | 10.4 g (86%) | 778.30 |
| 18 | | 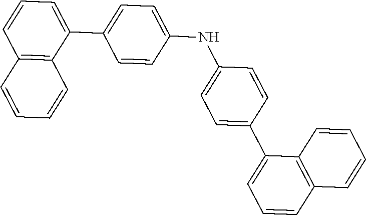 [SMILES: c1ccc2c(-c3ccc(Nc4ccc(-c5cccc6ccccc56)cc4)cc3)cccc2c1] | 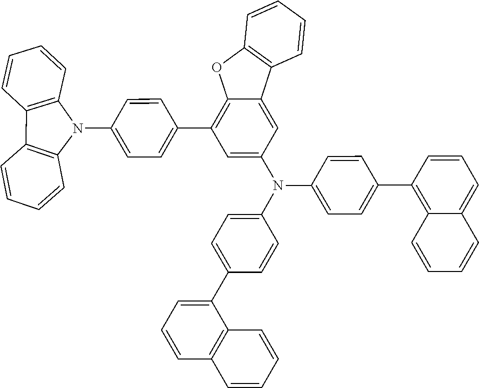 [SMILES: c1ccc2c(-c3ccc(N(c4ccc(-c5cccc6ccccc56)cc4)c4cc(-c5ccc(-n6c7ccccc7c7ccccc76)cc5)c5oc6ccccc6c5c4)cc3)cccc2c1] | 10.4 g (78%) | 828.31 |
| 19 | | 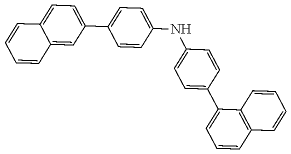 [SMILES: c1ccc2cc(-c3ccc(Nc4ccc(-c5cccc6ccccc56)cc4)cc3)ccc2c1] | 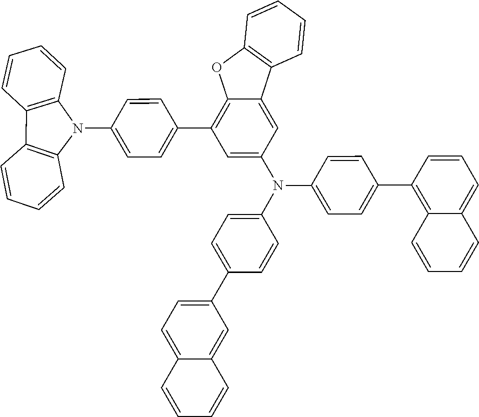 [SMILES: c1ccc2cc(-c3ccc(N(c4ccc(-c5cccc6ccccc56)cc4)c4cc(-c5ccc(-n6c7ccccc7c7ccccc76)cc5)c5oc6ccccc6c5c4)cc3)ccc2c1] | 9.5 g (74%) | 828.31 |
| 20 | | 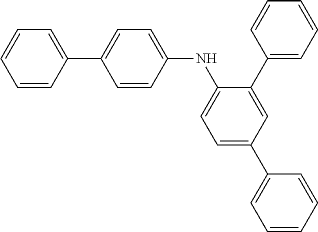 [SMILES: c1ccc(-c2ccc(Nc3ccc(-c4ccccc4)cc3-c3ccccc3)cc2)cc1] | 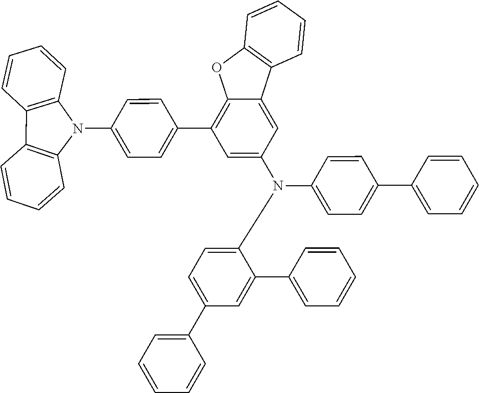 [SMILES: c1ccc(-c2ccc(N(c3cc(-c4ccc(-n5c6ccccc6c6ccccc65)cc4)c4oc5ccccc5c4c3)c3ccc(-c4ccccc4)cc3-c3ccccc3)cc2)cc1] | 9.5 g (76%) | 804.31 |
| 21 | | 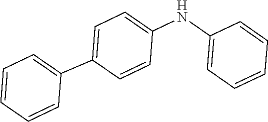 [SMILES: c1ccc(Nc2ccc(-c3ccccc3)cc2)cc1] | 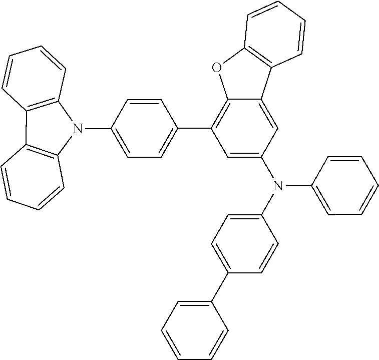 [SMILES: c1ccc(-c2ccc(N(c3ccccc3)c3cc(-c4ccc(-n5c6ccccc6c6ccccc65)cc4)c4oc5ccccc5c4c3)cc2)cc1] | 8.9 g (88%) | 625.25 |
| 22 | | 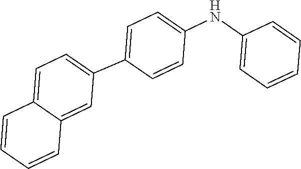 [SMILES: c1ccc(Nc2ccc(-c3ccc4ccccc4c3)cc2)cc1] | 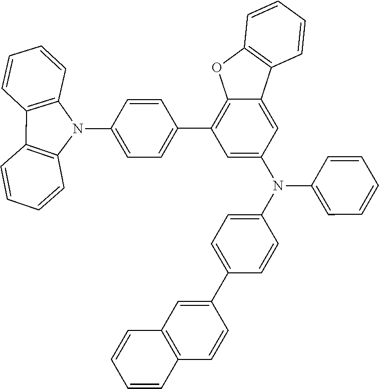 [SMILES: c1ccc(N(c2ccc(-c3ccc4ccccc4c3)cc2)c2cc(-c3ccc(-n4c5ccccc5c5ccccc54)cc3)c3oc4ccccc4c3c2)cc1] | 8.9 g (81%) | 702.27 |
| 23 | | 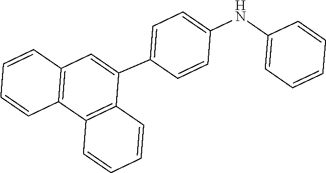 [SMILES: c1ccc(Nc2ccc(-c3cc4ccccc4c4ccccc34)cc2)cc1] | 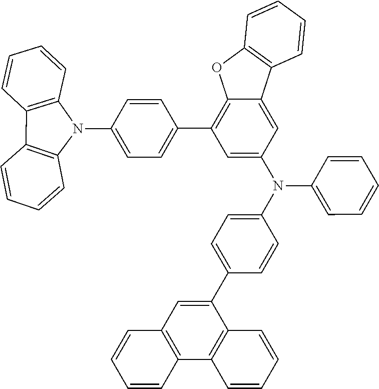 [SMILES: c1ccc(N(c2ccc(-c3cc4ccccc4c4ccccc34)cc2)c2cc(-c3ccc(-n4c5ccccc5c5ccccc54)cc3)c3oc4ccccc4c3c2)cc1] | 10.4 g (89%) | 752.28 |
| 24 | | 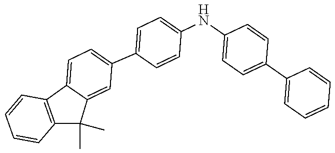 [SMILES: CC1(C)c2ccccc2-c2ccc(-c3ccc(Nc4ccc(-c5ccccc5)cc4)cc3)cc21] | 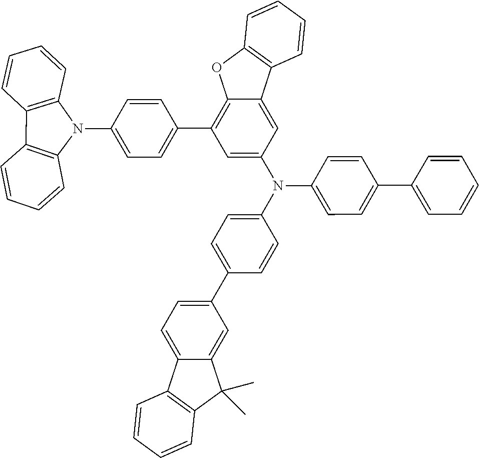 [SMILES: CC1(C)c2ccccc2-c2ccc(-c3ccc(N(c4ccc(-c5ccccc5)cc4)c4cc(-c5ccc(-n6c7ccccc7c7ccccc76)cc5)c5oc6ccccc6c5c4)cc3)cc21] | 10.9 g (83%) | 844.35 |
| 25 | | 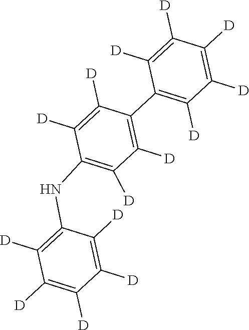 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] | 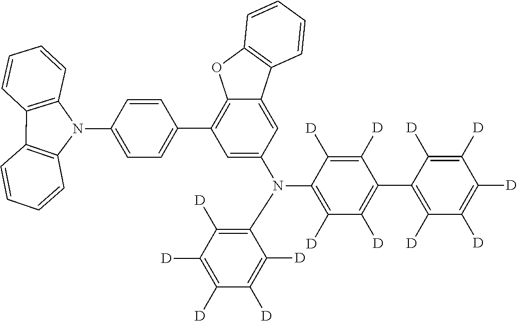 [SMILES: [2H]c1c([2H])c([2H])c(-c2c([2H])c([2H])c(N(c3cc(-c4ccc(-n5c6ccccc6c6ccccc65)cc4)c4oc5ccccc5c4c3)c3c([2H])c([2H])c([2H])c([2H])c3[2H])c([2H])c2[2H])c([2H])c1[2H]] | 8.5 g (82%) | 666.34 |
| 26 | | 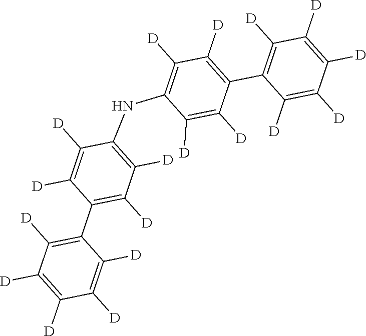 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] | 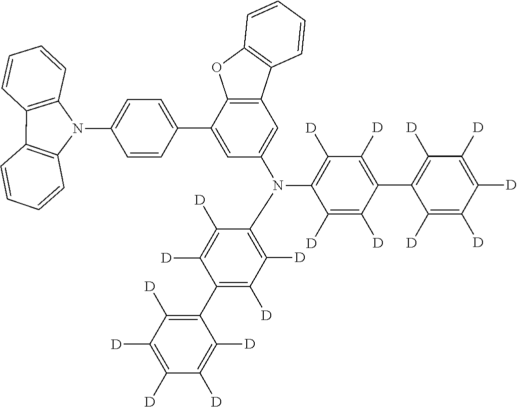 [SMILES: [2H]c1c([2H])c([2H])c(-c2c([2H])c([2H])c(N(c3cc(-c4ccc(-n5c6ccccc6c6ccccc65)cc4)c4oc5ccccc5c4c3)c3c([2H])c([2H])c(-c4c([2H])c([2H])c([2H])c([2H])c4[2H])c([2H])c3[2H])c([2H])c2[2H])c([2H])c1[2H]] | 8.8 g (76%) | 746.40 |
| 27 | | 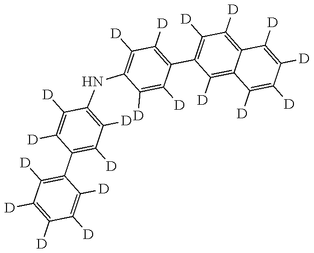 [SMILES: [2H]c1c([2H])c([2H])c(-c2c([2H])c([2H])c(Nc3c([2H])c([2H])c(-c4c([2H])c([2H])c5c([2H])c([2H])c([2H])c([2H])c5c4[2H])c([2H])c3[2H])c([2H])c2[2H])c([2H])c1[2H]] | 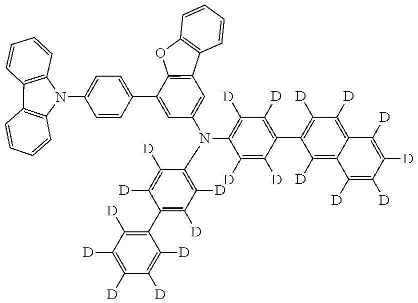 [SMILES: [2H]c1c([2H])c([2H])c(-c2c([2H])c([2H])c(N(c3cc(-c4ccc(-n5c6ccccc6c6ccccc65)cc4)c4oc5ccccc5c4c3)c3c([2H])c([2H])c(-c4c([2H])c([2H])c5c([2H])c([2H])c([2H])c([2H])c5c4[2H])c([2H])c3[2H])c([2H])c2[2H])c([2H])c1[2H]] | 10.1 g (81%) | 798.42 |
| 28 | 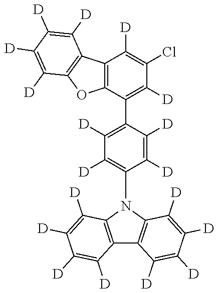 [SMILES: [2H]c1c([2H])c(-n2c3c([2H])c([2H])c([2H])c([2H])c3c3c([2H])c([2H])c([2H])c([2H])c32)c([2H])c([2H])c1-c1c([2H])c(Cl)c([2H])c2c1oc1c([2H])c([2H])c([2H])c([2H])c12] | 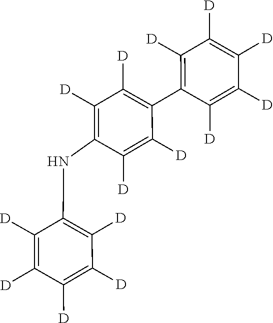 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] | 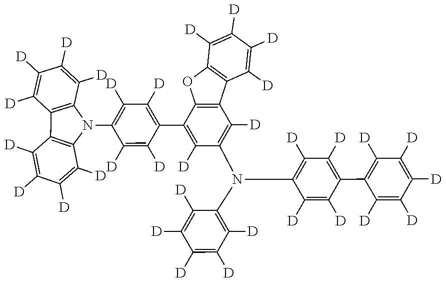 [SMILES: [2H]c1c([2H])c([2H])c(-c2c([2H])c([2H])c(N(c3c([2H])c([2H])c([2H])c([2H])c3[2H])c3c([2H])c(-c4c([2H])c([2H])c(-n5c6c([2H])c([2H])c([2H])c([2H])c6c6c([2H])c([2H])c([2H])c([2H])c65)c([2H])c4[2H])c4oc5c([2H])c([2H])c([2H])c([2H])c5c4c3[2H])c([2H])c2[2H])c([2H])c1[2H]] | 7.5 g (10.7 g) | 684.45 |
| 29 | | 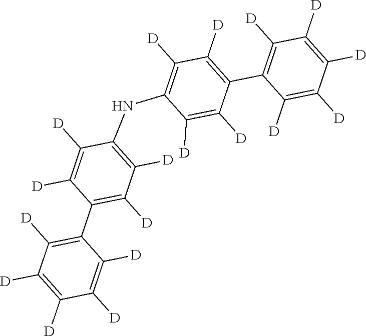 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] | 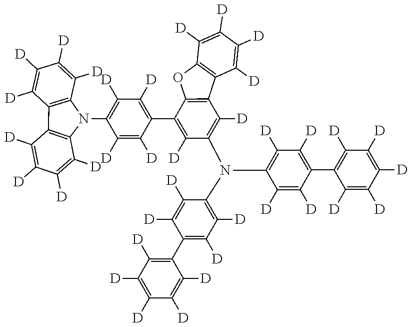 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c(-c4c([2H])c([2H])c(-n5c6c([2H])c([2H])c([2H])c([2H])c6c6c([2H])c([2H])c([2H])c([2H])c65)c([2H])c4[2H])c4oc5c([2H])c([2H])c([2H])c([2H])c5c4c3[2H])c([2H])c2[2H])c([2H])c1[2H]] | 9.2 g (77%) | 764.51 |
| 30 | | 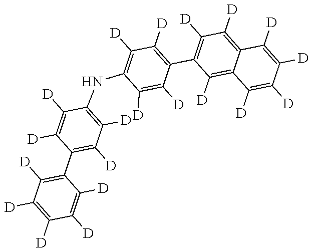 [SMILES: [2H]c1c([2H])c([2H])c(-c2c([2H])c([2H])c(Nc3c([2H])c([2H])c(-c4c([2H])c([2H])c5c([2H])c([2H])c([2H])c([2H])c5c4[2H])c([2H])c3[2H])c([2H])c2[2H])c([2H])c1[2H]] | 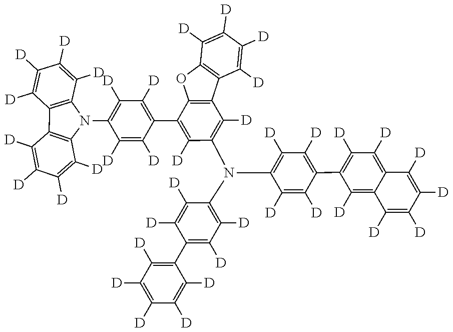 [SMILES: [2H]c1c([2H])c([2H])c(-c2c([2H])c([2H])c(N(c3c([2H])c([2H])c(-c4c([2H])c([2H])c5c([2H])c([2H])c([2H])c([2H])c5c4[2H])c([2H])c3[2H])c3c([2H])c(-c4c([2H])c([2H])c(-n5c6c([2H])c([2H])c([2H])c([2H])c6c6c([2H])c([2H])c([2H])c([2H])c65)c([2H])c4[2H])c4oc5c([2H])c([2H])c([2H])c([2H])c5c4c3[2H])c([2H])c2[2H])c([2H])c1[2H]] | 9.4 g (74%) | 816.54 |

TABLE 5

| | SUB 2-3 | | | |
|---|---|---|---|---|
| Compound | SUB 2-15 | SUB 1 | | |
| 31 | 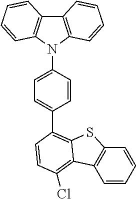 [SMILES: Clc1ccc(-c2ccc(-n3c4ccccc4c4ccccc43)cc2)c2sc3ccccc3c12] | 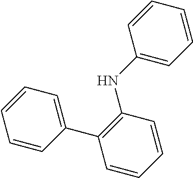 [SMILES: c1ccc(Nc2ccccc2-c2ccccc2)cc1] | | |
| 32 | | 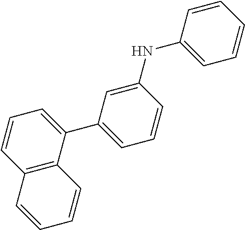 [SMILES: c1ccc(Nc2cccc(-c3cccc4ccccc34)c2)cc1] | | |
| 33 | | 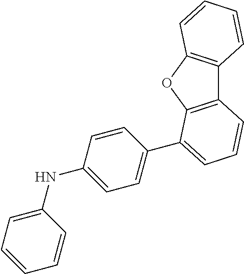 [SMILES: c1ccc(Nc2ccc(-c3cccc4c3oc3ccccc34)cc2)cc1] | | |
| 34 | | 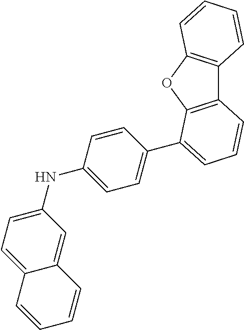 [SMILES: c1ccc2cc(Nc3ccc(-c4cccc5c4oc4ccccc45)cc3)ccc2c1] | | |
| 35 | | 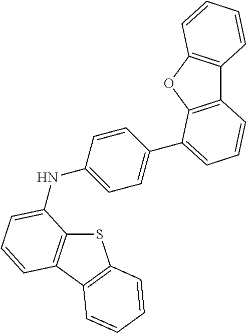 [SMILES: c1ccc2c(c1)oc1c(-c3ccc(Nc4cccc5c4sc4ccccc45)cc3)cccc12] | | |
| 36 | | 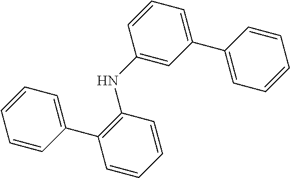 [SMILES: c1ccc(-c2cccc(Nc3ccccc3-c3ccccc3)c2)cc1] | | |
| 37 | | 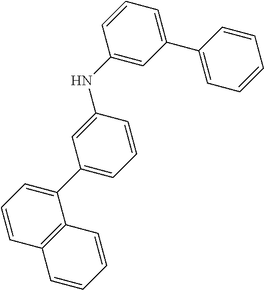 [SMILES: c1ccc(-c2cccc(Nc3cccc(-c4cccc5ccccc45)c3)c2)cc1] | | |
| 38 | | 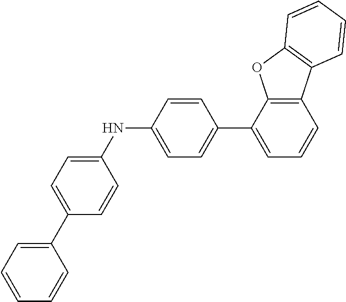 [SMILES: c1ccc(-c2ccc(Nc3ccc(-c4cccc5c4oc4ccccc45)cc3)cc2)cc1] | | |
| 39 | | 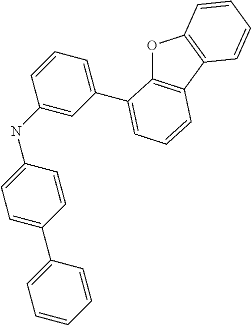 | | |
| 40 | | 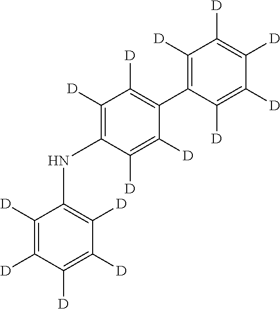 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] | | |
| 41 | | 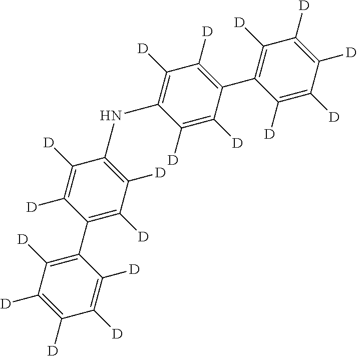 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] | | |
| 42 | 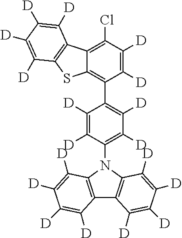 [SMILES: [2H]c1c([2H])c(-n2c3c([2H])c([2H])c([2H])c([2H])c3c3c([2H])c([2H])c([2H])c([2H])c32)c([2H])c([2H])c1-c1c([2H])c([2H])c(Cl)c2c1sc1c([2H])c([2H])c([2H])c([2H])c12] | 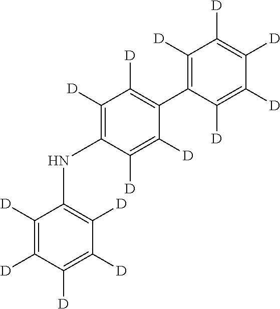 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] | | |
| 43 | | 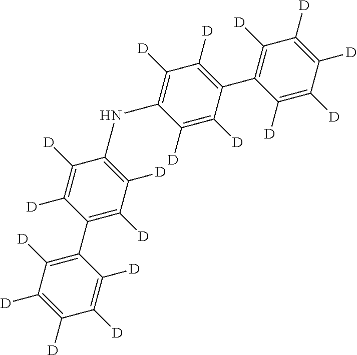 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] | | |
| | | | Obtained | |
| | | | amount | |
| | Compound | Product | (Yield) | [M + H]⁺ |
| | 31 | 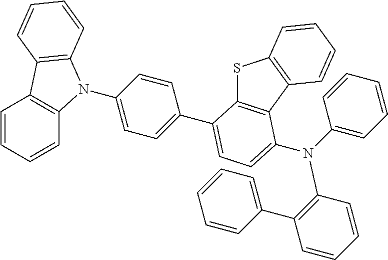 [SMILES: c1ccc(-c2ccccc2N(c2ccccc2)c2ccc(-c3ccc(-n4c5ccccc5c5ccccc54)cc3)c3sc4ccccc4c23)cc1] | 8.1 g (78%) | 668.23 |
| | 32 | 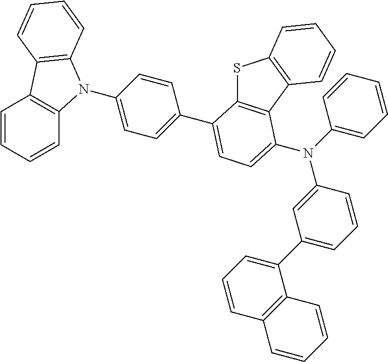 [SMILES: c1ccc(N(c2cccc(-c3cccc4ccccc34)c2)c2ccc(-c3ccc(-n4c5ccccc5c5ccccc54)cc3)c3sc4ccccc4c23)cc1] | 8.9 g (80%) | 718.24 |
| | 33 | 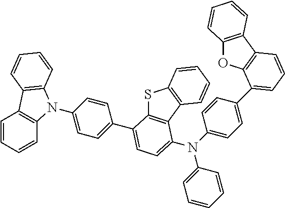 [SMILES: c1ccc(N(c2ccc(-c3cccc4c3oc3ccccc34)cc2)c2ccc(-c3ccc(-n4c5ccccc5c5ccccc54)cc3)c3sc4ccccc4c23)cc1] | 10.0 g (85%) | 758.24 |
| | 34 | 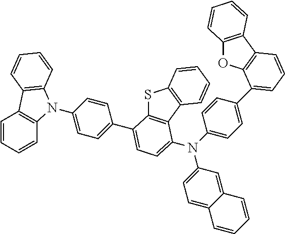 [SMILES: c1ccc2cc(N(c3ccc(-c4cccc5c4oc4ccccc45)cc3)c3ccc(-c4ccc(-n5c6ccccc6c6ccccc65)cc4)c4sc5ccccc5c34)ccc2c1] | 10.6 g (84%) | 808.25 |
| | 35 | 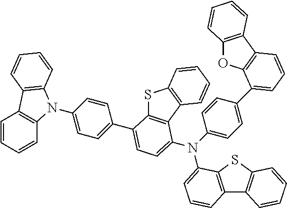 [SMILES: c1ccc2c(c1)oc1c(-c3ccc(N(c4cccc5c4sc4ccccc45)c4ccc(-c5ccc(-n6c7ccccc7c7ccccc76)cc5)c5sc6ccccc6c45)cc3)cccc12] | 10.6 g (79%) | 864.23 |
| | 36 | 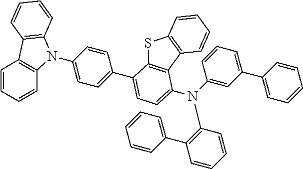 [SMILES: c1ccc(-c2cccc(N(c3ccccc3-c3ccccc3)c3ccc(-c4ccc(-n5c6ccccc6c6ccccc65)cc4)c4sc5ccccc5c34)c2)cc1] | 8.6 g (74%) | 744.26 |
| | 37 | 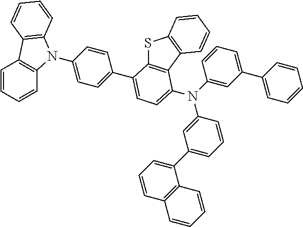 [SMILES: c1ccc(-c2cccc(N(c3cccc(-c4cccc5ccccc45)c3)c3ccc(-c4ccc(-n5c6ccccc6c6ccccc65)cc4)c4sc5ccccc5c34)c2)cc1] | 9.4 g (76%) | 794.28 |
| | 38 | 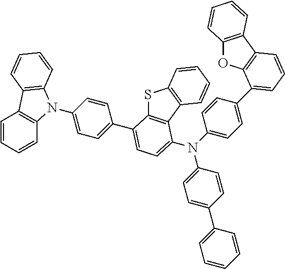 [SMILES: c1ccc(-c2ccc(N(c3ccc(-c4cccc5c4oc4ccccc45)cc3)c3ccc(-c4ccc(-n5c6ccccc6c6ccccc65)cc4)c4sc5ccccc5c34)cc2)cc1] | 10.9 g (84%) | 834.27 |
| | 39 | 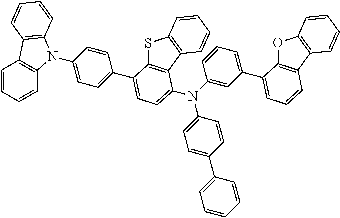 [SMILES: c1ccc(-c2ccc(N(c3cccc(-c4cccc5c4oc4ccccc45)c3)c3ccc(-c4ccc(-n5c6ccccc6c6ccccc65)cc4)c4sc5ccccc5c34)cc2)cc1] | 11.2 g (86%) | 834.27 |
| | 40 | 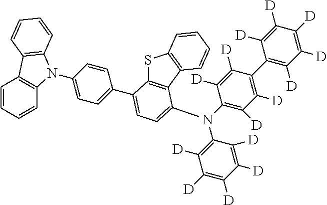 [SMILES: [2H]c1c([2H])c([2H])c(-c2c([2H])c([2H])c(N(c3c([2H])c([2H])c([2H])c([2H])c3[2H])c3ccc(-c4ccc(-n5c6ccccc6c6ccccc65)cc4)c4sc5ccccc5c34)c([2H])c2[2H])c([2H])c1[2H]] | 9.1 g (86%) | 682.32 |
| | 41 | 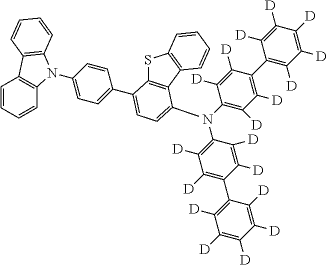 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3ccc(-c4ccc(-n5c6ccccc6c6ccccc65)cc4)c4sc5ccccc5c34)c([2H])c2[2H])c([2H])c1[2H]] | 10.0 g (84%) | 762.37 |
| | 42 | 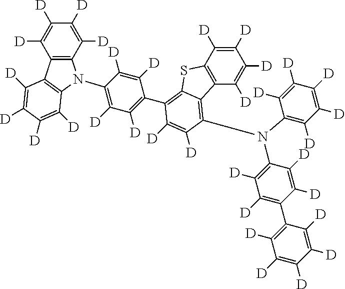 [SMILES: [2H]c1c([2H])c([2H])c(-c2c([2H])c([2H])c(N(c3c([2H])c([2H])c([2H])c([2H])c3[2H])c3c([2H])c([2H])c(-c4c([2H])c([2H])c(-n5c6c([2H])c([2H])c([2H])c([2H])c6c6c([2H])c([2H])c([2H])c([2H])c65)c([2H])c4[2H])c4sc5c([2H])c([2H])c([2H])c([2H])c5c34)c([2H])c2[2H])c([2H])c1[2H]] | 8.3 g (76%) | 700.43 |
| | 43 | 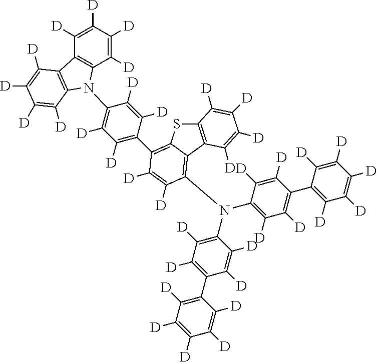 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c(-c4c([2H])c([2H])c(-n5c6c([2H])c([2H])c([2H])c([2H])c6c6c([2H])c([2H])c([2H])c([2H])c65)c([2H])c4[2H])c4sc5c([2H])c([2H])c([2H])c([2H])c5c34)c([2H])c2[2H])c([2H])c1[2H]] | 9.6 g (79%) | 780.49 |

TABLE 6

| | SUB 2-4 | |
|---|---|---|
| Compound | SUB 2-16 | SUB 1 |
| 44 | 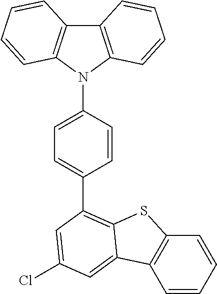 [SMILES: Clc1cc(-c2ccc(-n3c4ccccc4c4ccccc43)cc2)c2sc3ccccc3c2c1] | 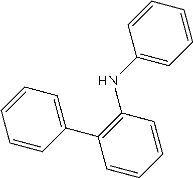 [SMILES: c1ccc(Nc2ccccc2-c2ccccc2)cc1] |
| 45 | | 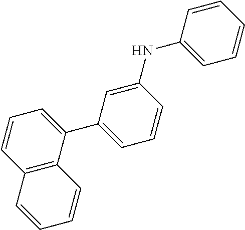 [SMILES: c1ccc(Nc2cccc(-c3cccc4ccccc34)c2)cc1] |
| 46 | | 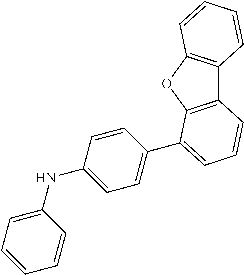 [SMILES: c1ccc(Nc2ccc(-c3cccc4c3oc3ccccc34)cc2)cc1] |
| 47 | | 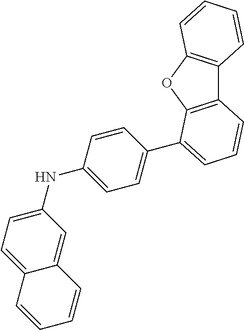 [SMILES: c1ccc2cc(Nc3ccc(-c4cccc5c4oc4ccccc45)cc3)ccc2c1] |
| 48 | | 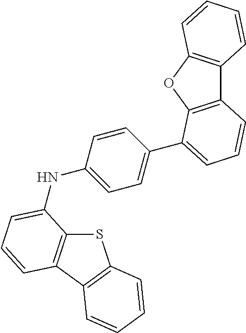 [SMILES: c1ccc2c(c1)oc1c(-c3ccc(Nc4cccc5c4sc4ccccc45)cc3)cccc12] |
| 49 | | 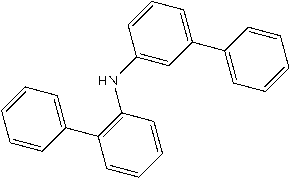 [SMILES: c1ccc(-c2cccc(Nc3ccccc3-c3ccccc3)c2)cc1] |
| 50 | | 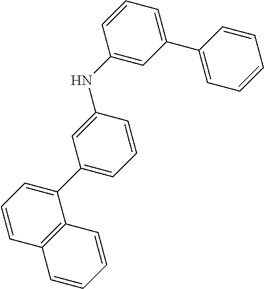 [SMILES: c1ccc(-c2cccc(Nc3cccc(-c4cccc5ccccc45)c3)c2)cc1] |
| 51 | | 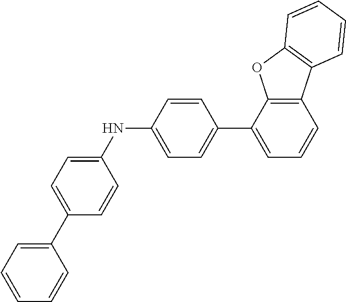 [SMILES: c1ccc(-c2ccc(Nc3ccc(-c4cccc5c4oc4ccccc45)cc3)cc2)cc1] |
| 52 | | 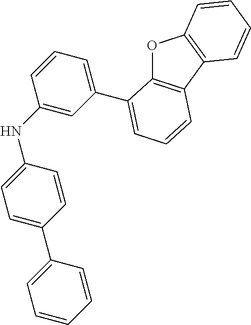 [SMILES: c1ccc(-c2ccc(Nc3cccc(-c4cccc5c4oc4ccccc45)c3)cc2)cc1] |
| 53 | | 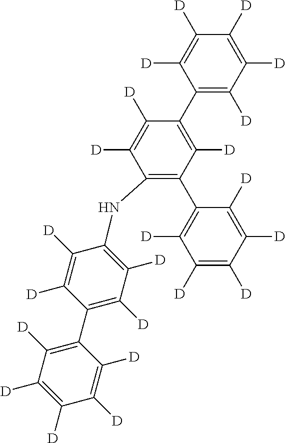 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3-c3c([2H])c([2H])c([2H])c([2H])c3[2H])c([2H])c2[2H])c([2H])c1[2H]] |
| 54 | | 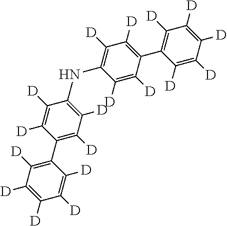 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] |
| 55 | 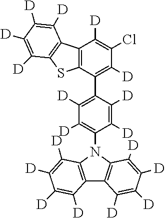 [SMILES: [2H]c1c([2H])c(-n2c3c([2H])c([2H])c([2H])c([2H])c3c3c([2H])c([2H])c([2H])c([2H])c32)c([2H])c([2H])c1-c1c([2H])c(Cl)c([2H])c2c1sc1c([2H])c([2H])c([2H])c([2H])c12] | 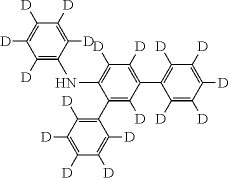 [SMILES: [2H]c1c([2H])c([2H])c(Nc2c([2H])c([2H])c(-c3c([2H])c([2H])c([2H])c([2H])c3[2H])c([2H])c2-c2c([2H])c([2H])c([2H])c([2H])c2[2H])c([2H])c1[2H]] |
| 56 | | 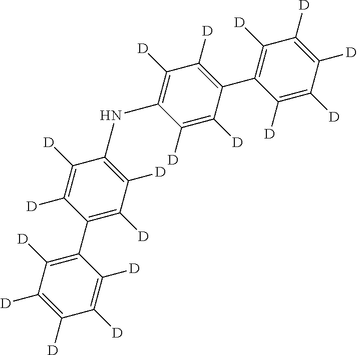 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] |

| | | Obtained | |
|---|---|---|---|
| | | amount | |
| Compound | Product | (Yield) | [M + H]⁺ |
| 44 | 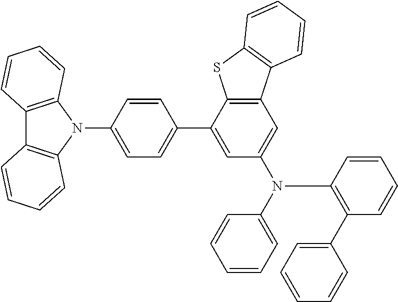 [SMILES: c1ccc(-c2ccccc2N(c2ccccc2)c2cc(-c3ccc(-n4c5ccccc5c5ccccc54)cc3)c3sc4ccccc4c3c2)cc1] | 7.9 g (76%) | 668.23 |
| 45 | 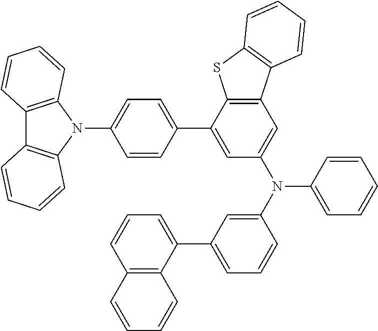 [SMILES: c1ccc(N(c2cccc(-c3cccc4ccccc34)c2)c2cc(-c3ccc(-n4c5ccccc5c5ccccc54)cc3)c3sc4ccccc4c3c2)cc1] | 8.4 g (75%) | 718.24 |
| 46 | 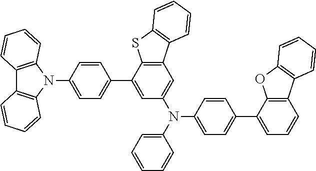 [SMILES: c1ccc(N(c2ccc(-c3cccc4c3oc3ccccc34)cc2)c2cc(-c3ccc(-n4c5ccccc5c5ccccc54)cc3)c3sc4ccccc4c3c2)cc1] | 9.3 g (79%) | 758.24 |
| 47 | 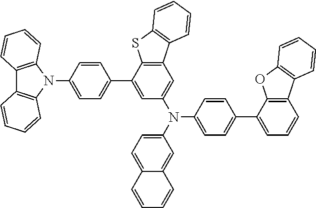 [SMILES: c1ccc2cc(N(c3ccc(-c4cccc5c4oc4ccccc45)cc3)c3cc(-c4ccc(-n5c6ccccc6c6ccccc65)cc4)c4sc5ccccc5c4c3)ccc2c1] | 10.1 g (80%) | 808.25 |
| 48 | 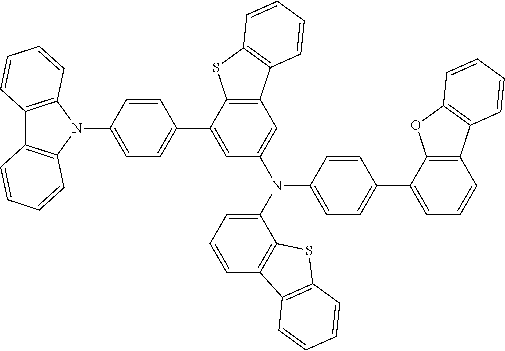 [SMILES: c1ccc2c(c1)oc1c(-c3ccc(N(c4cc(-c5ccc(-n6c7ccccc7c7ccccc76)cc5)c5sc6ccccc6c5c4)c4cccc5c4sc4ccccc45)cc3)cccc12] | 10.0 g (74%) | 864.23 |
| 49 | 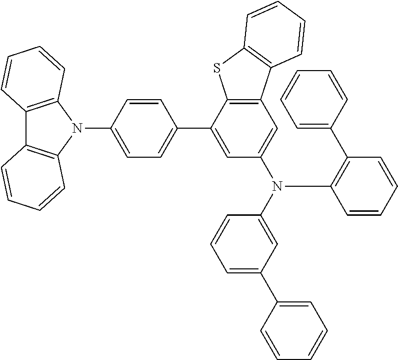 [SMILES: c1ccc(-c2cccc(N(c3cc(-c4ccc(-n5c6ccccc6c6ccccc65)cc4)c4sc5ccccc5c4c3)c3ccccc3-c3ccccc3)c2)cc1] | 8.7 g (75%) | 744.26 |
| 50 | 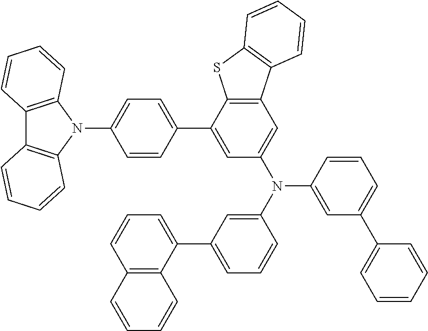 [SMILES: c1ccc(-c2cccc(N(c3cccc(-c4cccc5ccccc45)c3)c3cc(-c4ccc(-n5c6ccccc6c6ccccc65)cc4)c4sc5ccccc5c4c3)c2)cc1] | 9.1 g (74%) | 794.28 |
| 51 | 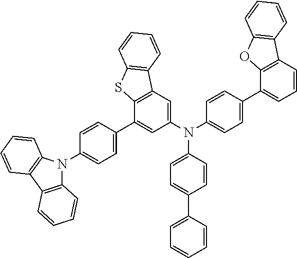 [SMILES: c1ccc(-c2ccc(N(c3ccc(-c4cccc5c4oc4ccccc45)cc3)c3cc(-c4ccc(-n5c6ccccc6c6ccccc65)cc4)c4sc5ccccc5c4c3)cc2)cc1] | 10.0 g (77%) | 834.27 |
| 52 | 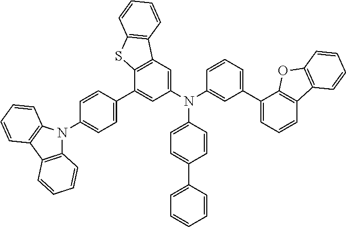 [SMILES: c1ccc(-c2ccc(N(c3cccc(-c4cccc5c4oc4ccccc45)c3)c3cc(-c4ccc(-n5c6ccccc6c6ccccc65)cc4)c4sc5ccccc5c4c3)cc2)cc1] | 10.3 g (79%) | 834.27 |
| 53 | 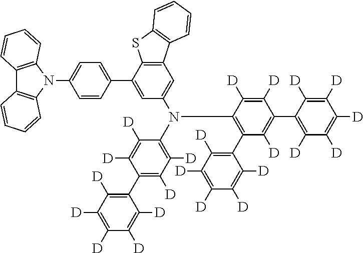 [SMILES: [2H]c1c([2H])c([2H])c(-c2c([2H])c([2H])c(N(c3cc(-c4ccc(-n5c6ccccc6c6ccccc65)cc4)c4sc5ccccc5c4c3)c3c([2H])c([2H])c(-c4c([2H])c([2H])c([2H])c([2H])c4[2H])c([2H])c3-c3c([2H])c([2H])c([2H])c([2H])c3[2H])c([2H])c2[2H])c([2H])c1[2H]] | 10.7 g (80%) | 826.45 |
| 54 | 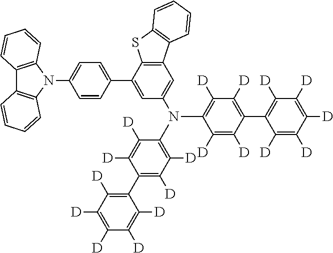 [SMILES: [2H]c1c([2H])c([2H])c(-c2c([2H])c([2H])c(N(c3cc(-c4ccc(-n5c6ccccc6c6ccccc65)cc4)c4sc5ccccc5c4c3)c3c([2H])c([2H])c(-c4c([2H])c([2H])c([2H])c([2H])c4[2H])c([2H])c3[2H])c([2H])c2[2H])c([2H])c1[2H]] | 8.4 g (71%) | 762.37 |
| 55 | 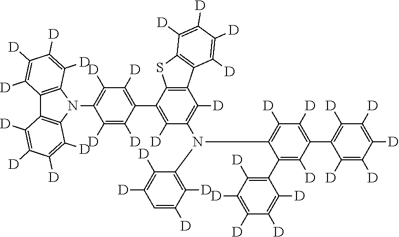 [SMILES: [2H]c1cc([2H])c([2H])c(N(c2c([2H])c([2H])c(-c3c([2H])c([2H])c([2H])c([2H])c3[2H])c([2H])c2-c2c([2H])c([2H])c([2H])c([2H])c2[2H])c2c([2H])c(-c3c([2H])c([2H])c(-n4c5c([2H])c([2H])c([2H])c([2H])c5c5c([2H])c([2H])c([2H])c([2H])c54)c([2H])c3[2H])c3sc4c([2H])c([2H])c([2H])c([2H])c4c3c2[2H])c1[2H]] | 8.9 g (73%) | 780.49 |
| 56 | 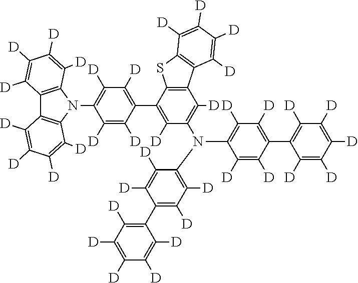 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c(-c4c([2H])c([2H])c(-n5c6c([2H])c([2H])c([2H])c([2H])c6c6c([2H])c([2H])c([2H])c([2H])c65)c([2H])c4[2H])c4sc5c([2H])c([2H])c([2H])c([2H])c5c4c3[2H])c([2H])c2[2H])c([2H])c1[2H]] | 8.7 g (72%) | 780.49 |

TABLE 7

| | SUB 2-5 | | | |
|---|---|---|---|---|
| Compound | SUB 2-17 | SUB 1 | | |
| 57 | 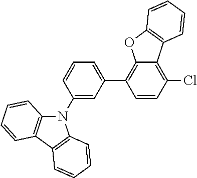 [SMILES: Clc1ccc(-c2cccc(-n3c4ccccc4c4ccccc43)c2)c2oc3ccccc3c12] | 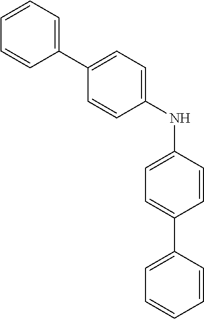 [SMILES: c1ccc(-c2ccc(Nc3ccc(-c4ccccc4)cc3)cc2)cc1] | | |
| 58 | | 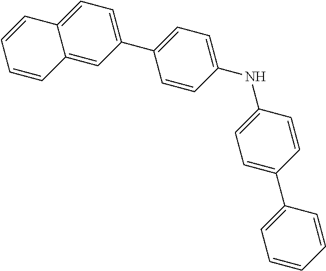 [SMILES: c1ccc(-c2ccc(Nc3ccc(-c4ccc5ccccc5c4)cc3)cc2)cc1] | | |
| 59 | | 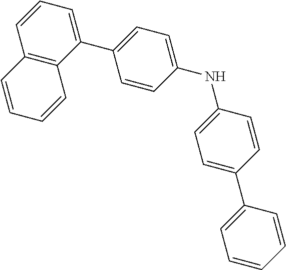 [SMILES: c1ccc(-c2ccc(Nc3ccc(-c4cccc5ccccc45)cc3)cc2)cc1] | | |
| 60 | | 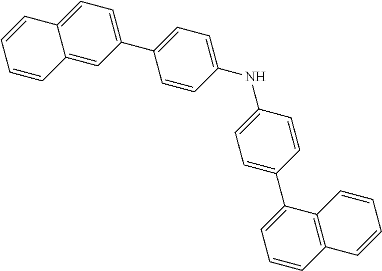 [SMILES: c1ccc2cc(-c3ccc(Nc4ccc(-c5cccc6ccccc56)cc4)cc3)ccc2c1] | | |
| 61 | | 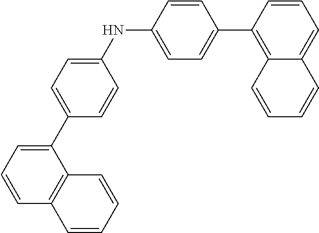 [SMILES: c1ccc2c(-c3ccc(Nc4ccc(-c5cccc6ccccc56)cc4)cc3)cccc2c1] | | |
| 62 | | 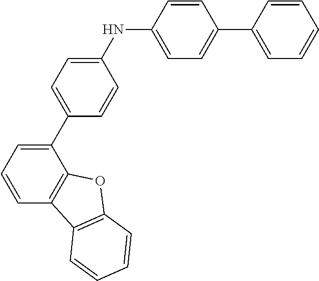 [SMILES: c1ccc(-c2ccc(Nc3ccc(-c4cccc5c4oc4ccccc45)cc3)cc2)cc1] | | |
| 63 | | 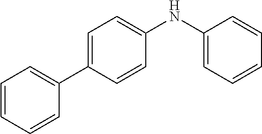 [SMILES: c1ccc(Nc2ccc(-c3ccccc3)cc2)cc1] | | |
| 64 | | 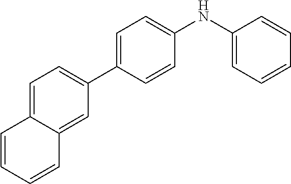 [SMILES: c1ccc(Nc2ccc(-c3ccc4ccccc4c3)cc2)cc1] | | |
| 65 | | 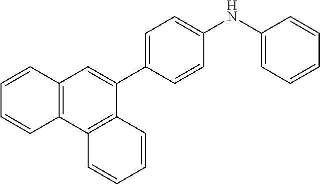 [SMILES: c1ccc(Nc2ccc(-c3cc4ccccc4c4ccccc34)cc2)cc1] | | |
| 66 | | 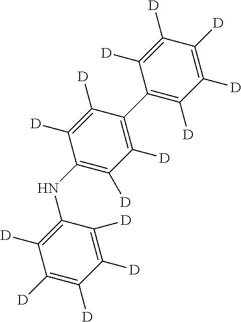 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] | | |
| 67 | | 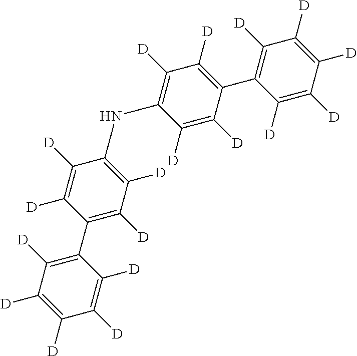 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] | | |
| 68 | | 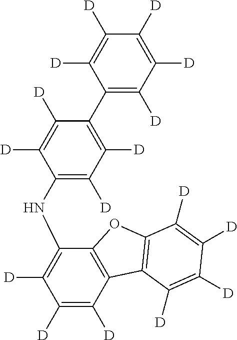 [SMILES: [2H]c1c([2H])c([2H])c(-c2c([2H])c([2H])c(Nc3c([2H])c([2H])c([2H])c4c3oc3c([2H])c([2H])c([2H])c([2H])c34)c([2H])c2[2H])c([2H])c1[2H]] | | |
| 69 | 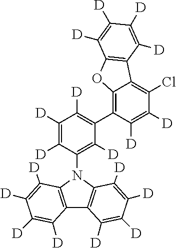 [SMILES: [2H]c1c([2H])c(-c2c([2H])c([2H])c(Cl)c3c2oc2c([2H])c([2H])c([2H])c([2H])c23)c([2H])c(-n2c3c([2H])c([2H])c([2H])c([2H])c3c3c([2H])c([2H])c([2H])c([2H])c32)c1[2H]] | 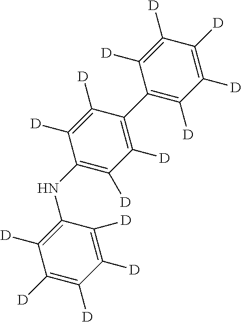 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] | | |
| 70 | | 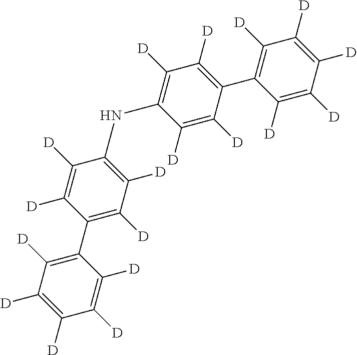 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] | | |
| 71 | | 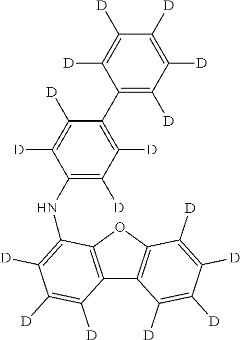 [SMILES: [2H]c1c([2H])c([2H])c(-c2c([2H])c([2H])c(Nc3c([2H])c([2H])c([2H])c4c3oc3c([2H])c([2H])c([2H])c([2H])c34)c([2H])c2[2H])c([2H])c1[2H]] | | |
| | | | Obtained | |
| | | | amount | |
| | Compound | Product | (Yield) | [M + H]⁺ |
| | 57 | 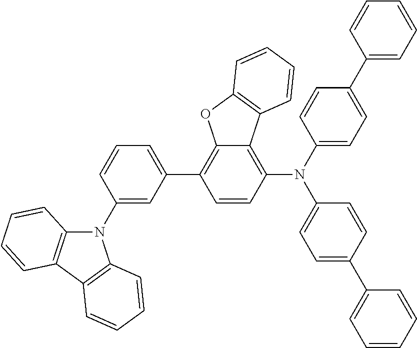 [SMILES: c1ccc(-c2ccc(N(c3ccc(-c4ccccc4)cc3)c3ccc(-c4cccc(-n5c6ccccc6c6ccccc65)c4)c4oc5ccccc5c34)cc2)cc1] | 9.8 g (86%) | 729.29 |
| | 58 | 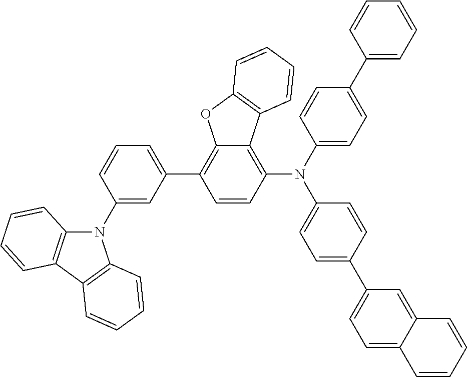 [SMILES: c1ccc(-c2ccc(N(c3ccc(-c4ccc5ccccc5c4)cc3)c3ccc(-c4cccc(-n5c6ccccc6c6ccccc65)c4)c4oc5ccccc5c34)cc2)cc1] | 9.8 g (81%) | 778.30 |
| | 59 | 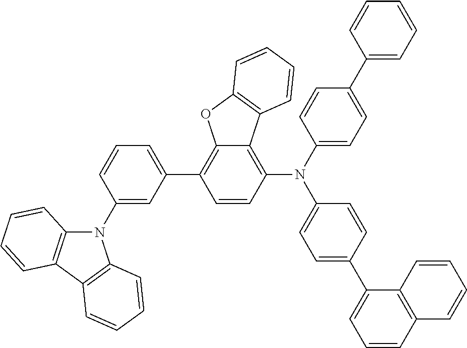 [SMILES: c1ccc(-c2ccc(N(c3ccc(-c4cccc5ccccc45)cc3)c3ccc(-c4cccc(-n5c6ccccc6c6ccccc65)c4)c4oc5ccccc5c34)cc2)cc1] | 9.9 g (82%) | 778.30 |
| | 60 | 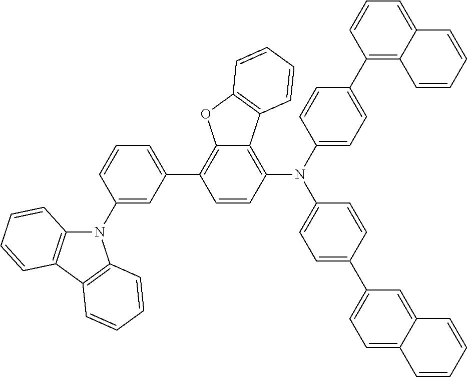 [SMILES: c1cc(-c2ccc(N(c3ccc(-c4ccc5ccccc5c4)cc3)c3ccc(-c4cccc5ccccc45)cc3)c3c2oc2ccccc23)cc(-n2c3ccccc3c3ccccc32)c1] | 10.6 g (82%) | 828.31 |
| | 61 | 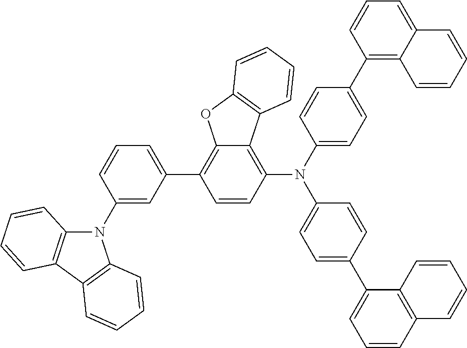 [SMILES: c1cc(-c2ccc(N(c3ccc(-c4cccc5ccccc45)cc3)c3ccc(-c4cccc5ccccc45)cc3)c3c2oc2ccccc23)cc(-n2c3ccccc3c3ccccc32)c1] | 10.8 g (84%) | 828.31 |
| | 62 | 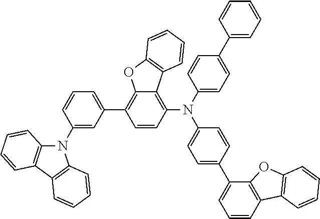 [SMILES: c1ccc(-c2ccc(N(c3ccc(-c4cccc5c4oc4ccccc45)cc3)c3ccc(-c4cccc(-n5c6ccccc6c6ccccc65)c4)c4oc5ccccc5c34)cc2)cc1] | 10.6 g (83%) | 818.29 |
| | 63 | 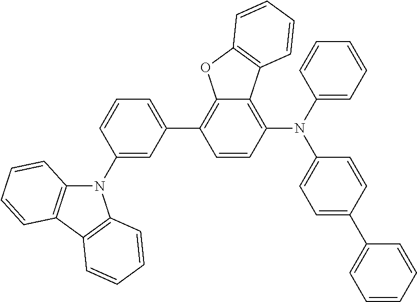 [SMILES: c1ccc(-c2ccc(N(c3ccccc3)c3ccc(-c4cccc(-n5c6ccccc6c6ccccc65)c4)c4oc5ccccc5c34)cc2)cc1] | 8.9 g (88%) | 652.25 |
| | 64 | 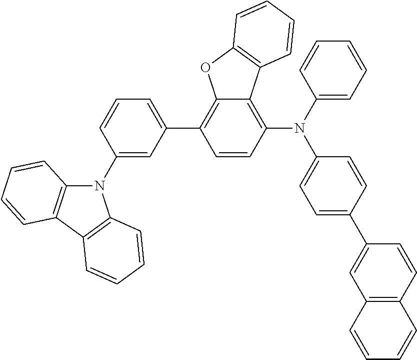 [SMILES: c1ccc(N(c2ccc(-c3ccc4ccccc4c3)cc2)c2ccc(-c3cccc(-n4c5ccccc5c5ccccc54)c3)c3oc4ccccc4c23)cc1] | 8.6 g (79%) | 702.27 |
| | 65 | 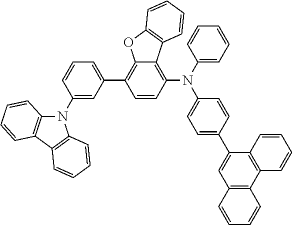 [SMILES: c1ccc(N(c2ccc(-c3cc4ccccc4c4ccccc34)cc2)c2ccc(-c3cccc(-n4c5ccccc5c5ccccc54)c3)c3oc4ccccc4c23)cc1] | 9.1 g (78%) | 752.28 |
| | 66 | 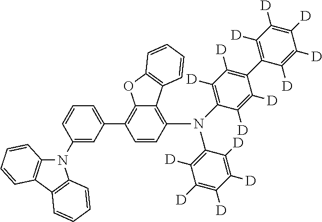 [SMILES: [2H]c1c([2H])c([2H])c(-c2c([2H])c([2H])c(N(c3c([2H])c([2H])c([2H])c([2H])c3[2H])c3ccc(-c4cccc(-n5c6ccccc6c6ccccc65)c4)c4oc5ccccc5c34)c([2H])c2[2H])c([2H])c1[2H]] | 8.7 g (84%) | 666.34 |
| | 67 | 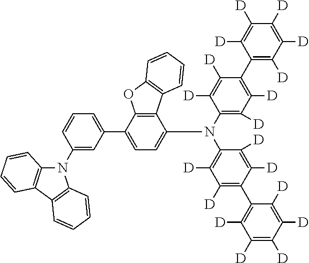 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3ccc(-c4cccc(-n5c6ccccc6c6ccccc65)c4)c4oc5ccccc5c34)c([2H])c2[2H])c([2H])c1[2H]] | 9.4 g (81%) | 746.40 |
| | 68 | 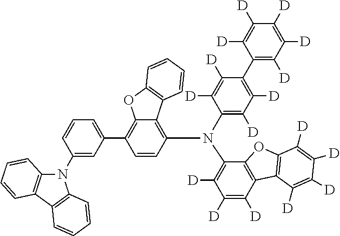 [SMILES: [2H]c1c([2H])c([2H])c(-c2c([2H])c([2H])c(N(c3c([2H])c([2H])c([2H])c4c3oc3c([2H])c([2H])c([2H])c([2H])c34)c3ccc(-c4cccc(-n5c6ccccc6c6ccccc65)c4)c4oc5ccccc5c34)c([2H])c2[2H])c([2H])c1[2H]] | 8.7 g (74%) | 758.36 |
| | 69 | 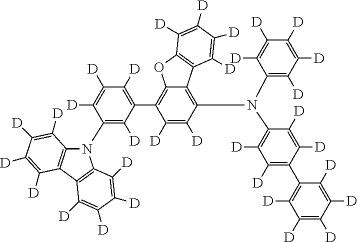 [SMILES: [2H]c1c([2H])c([2H])c(-c2c([2H])c([2H])c(N(c3c([2H])c([2H])c([2H])c([2H])c3[2H])c3c([2H])c([2H])c(-c4c([2H])c([2H])c([2H])c(-n5c6c([2H])c([2H])c([2H])c([2H])c6c6c([2H])c([2H])c([2H])c([2H])c65)c4[2H])c4oc5c([2H])c([2H])c([2H])c([2H])c5c34)c([2H])c2[2H])c([2H])c1[2H]] | 8.5 g (80%) | 684.45 |
| | 70 | 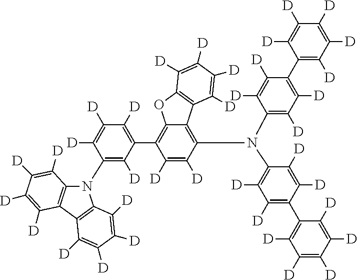 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c(-c4c([2H])c([2H])c([2H])c(-n5c6c([2H])c([2H])c([2H])c([2H])c6c6c([2H])c([2H])c([2H])c([2H])c65)c4[2H])c4oc5c([2H])c([2H])c([2H])c([2H])c5c34)c([2H])c2[2H])c([2H])c1[2H]] | 9.9 g (83%) | 764.51 |
| | 71 | 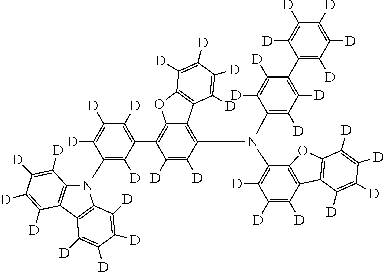 [SMILES: [2H]c1c([2H])c([2H])c(-c2c([2H])c([2H])c(N(c3c([2H])c([2H])c([2H])c4c3oc3c([2H])c([2H])c([2H])c([2H])c34)c3c([2H])c([2H])c(-c4c([2H])c([2H])c([2H])c(-n5c6c([2H])c([2H])c([2H])c([2H])c6c6c([2H])c([2H])c([2H])c([2H])c65)c4[2H])c4oc5c([2H])c([2H])c([2H])c([2H])c5c34)c([2H])c2[2H])c([2H])c1[2H]] | 9.5 g (79%) | 776.48 |

TABLE 8

| | SUB 2-6 | |
|---|---|---|
| Compound | SUB 2-18 | SUB 1 |
| 72 | 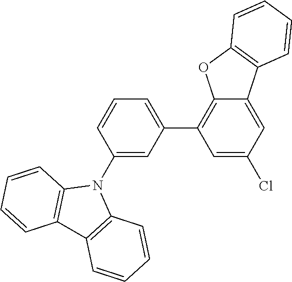 [SMILES: Clc1cc(-c2cccc(-n3c4ccccc4c4ccccc43)c2)c2oc3ccccc3c2c1] | 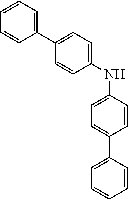 [SMILES: c1ccc(-c2ccc(Nc3ccc(-c4ccccc4)cc3)cc2)cc1] |
| 73 | | 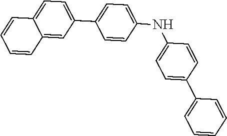 [SMILES: c1ccc(-c2ccc(Nc3ccc(-c4ccc5ccccc5c4)cc3)cc2)cc1] |
| 74 | | 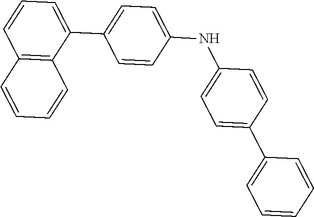 [SMILES: c1ccc(-c2ccc(Nc3ccc(-c4cccc5ccccc45)cc3)cc2)cc1] |
| 75 | | 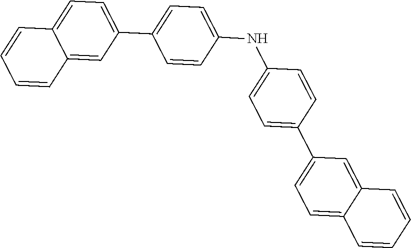 [SMILES: c1ccc2cc(-c3ccc(Nc4ccc(-c5ccc6ccccc6c5)cc4)cc3)ccc2c1] |
| 76 | | 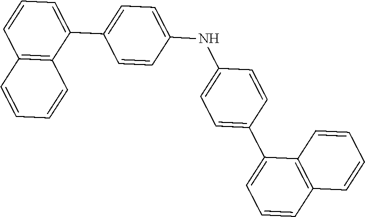 [SMILES: c1ccc2c(-c3ccc(Nc4ccc(-c5cccc6ccccc56)cc4)cc3)cccc2c1] |
| 77 | | 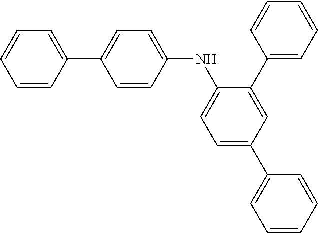 [SMILES: c1ccc(-c2ccc(Nc3ccc(-c4ccccc4)cc3-c3ccccc3)cc2)cc1] |
| 78 | | 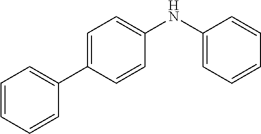 [SMILES: c1ccc(Nc2ccc(-c3ccccc3)cc2)cc1] |
| 79 | | 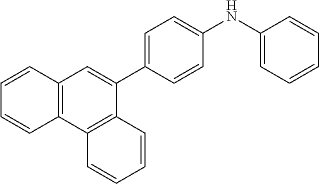 [SMILES: c1ccc(Nc2ccc(-c3cc4ccccc4c4ccccc34)cc2)cc1] |
| 80 | | 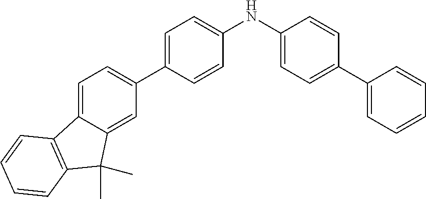 [SMILES: CC1(C)c2ccccc2-c2ccc(-c3ccc(Nc4ccc(-c5ccccc5)cc4)cc3)cc21] |
| 81 | | 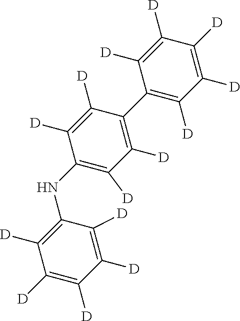 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] |
| 82 | | 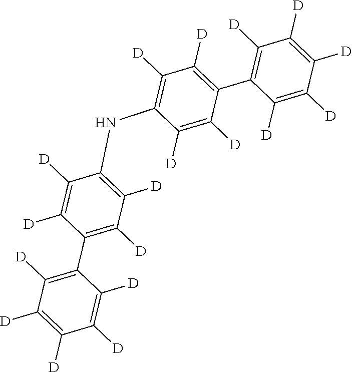 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] |
| 83 | | 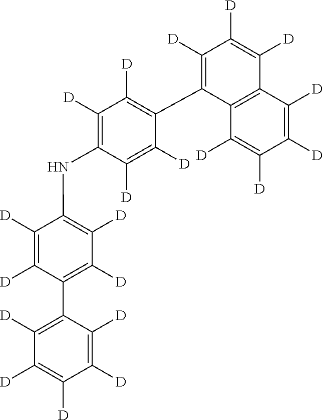 [SMILES: [2H]c1c([2H])c([2H])c(-c2c([2H])c([2H])c(Nc3c([2H])c([2H])c(-c4c([2H])c([2H])c([2H])c5c([2H])c([2H])c([2H])c([2H])c45)c([2H])c3[2H])c([2H])c2[2H])c([2H])c1[2H]] |
| 84 | 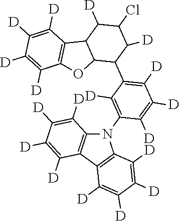 [SMILES: [2H]c1c([2H])c([2H])c2c(c1[2H])OC1C(c3c([2H])c([2H])c([2H])c(-n4c5c([2H])c([2H])c([2H])c([2H])c5c5c([2H])c([2H])c([2H])c([2H])c54)c3[2H])C([2H])C(Cl)C([2H])C21] | 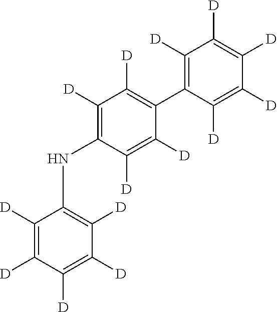 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] |
| 85 | | 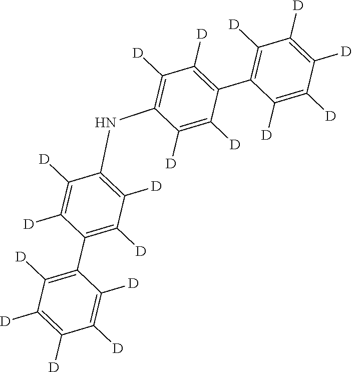 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] |
| 86 | | 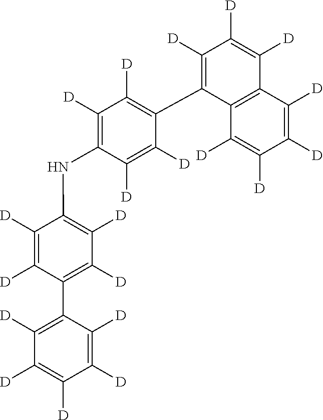 [SMILES: [2H]c1c([2H])c([2H])c(-c2c([2H])c([2H])c(Nc3c([2H])c([2H])c(-c4c([2H])c([2H])c([2H])c5c([2H])c([2H])c([2H])c([2H])c45)c([2H])c3[2H])c([2H])c2[2H])c([2H])c1[2H]] |

| | | Obtained | |
|---|---|---|---|
| | | amount | |
| Compound | Product | (Yield) | [M + H]⁺ |
| 72 | 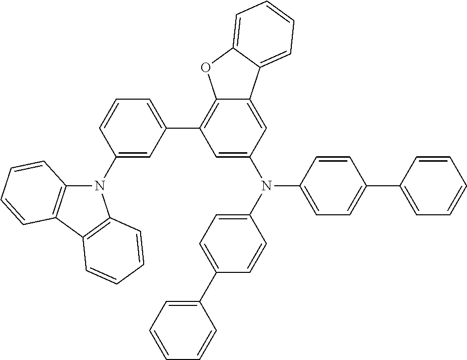 [SMILES: c1ccc(-c2ccc(N(c3ccc(-c4ccccc4)cc3)c3cc(-c4cccc(-n5c6ccccc6c6ccccc65)c4)c4oc5ccccc5c4c3)cc2)cc1] | 8.6 g (76%) | 728.28 |
| 73 | 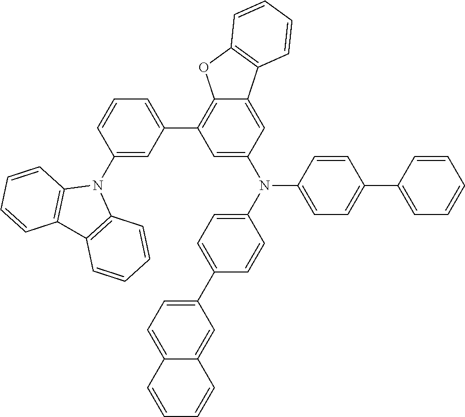 [SMILES: c1ccc(-c2ccc(N(c3ccc(-c4ccc5ccccc5c4)cc3)c3cc(-c4cccc(-n5c6ccccc6c6ccccc65)c4)c4oc5ccccc5c4c3)cc2)cc1] | 8.7 g (72%) | 778.30 |
| 74 | 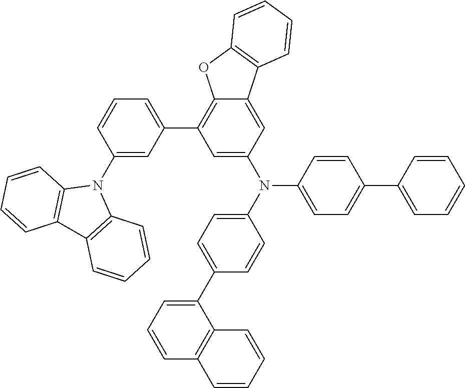 [SMILES: c1ccc(-c2ccc(N(c3ccc(-c4cccc5ccccc45)cc3)c3cc(-c4cccc(-n5c6ccccc6c6ccccc65)c4)c4oc5ccccc5c4c3)cc2)cc1] | 9.1 g (75%) | 778.30 |
| 75 | 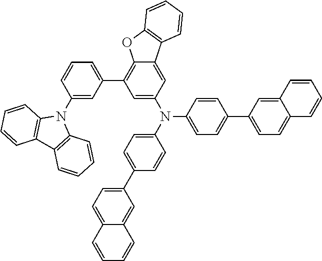 [SMILES: c1cc(-c2cc(N(c3ccc(-c4ccc5ccccc5c4)cc3)c3ccc(-c4ccc5ccccc5c4)cc3)cc3c2oc2ccccc23)cc(-n2c3ccccc3c3ccccc32)c1] | 10.2 g (79%) | 828.31 |
| 76 | 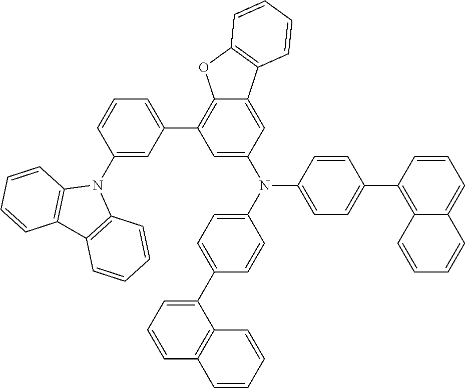 [SMILES: c1cc(-c2cc(N(c3ccc(-c4cccc5ccccc45)cc3)c3ccc(-c4cccc5ccccc45)cc3)cc3c2oc2ccccc23)cc(-n2c3ccccc3c3ccccc32)c1] | 10.4 g (81%) | 828.31 |
| 77 | 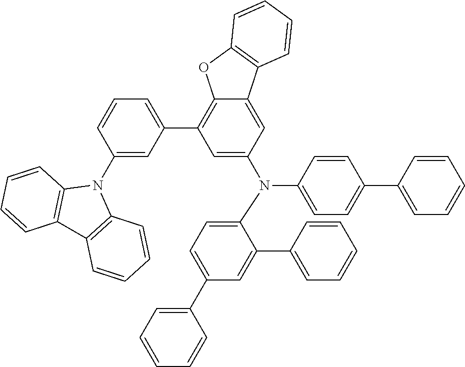 [SMILES: c1ccc(-c2ccc(N(c3cc(-c4cccc(-n5c6ccccc6c6ccccc65)c4)c4oc5ccccc5c4c3)c3ccc(-c4ccccc4)cc3-c3ccccc3)cc2)cc1] | 10.4 g (83%) | 804.31 |
| 78 | 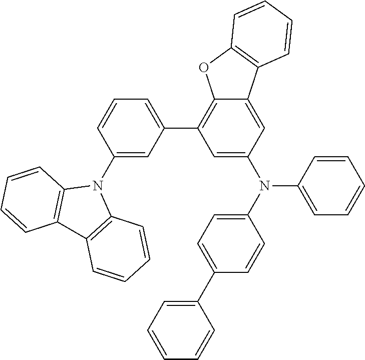 [SMILES: c1ccc(-c2ccc(N(c3ccccc3)c3cc(-c4cccc(-n5c6ccccc6c6ccccc65)c4)c4oc5ccccc5c4c3)cc2)cc1] | 8.1 g (80%) | 652.25 |
| 79 | 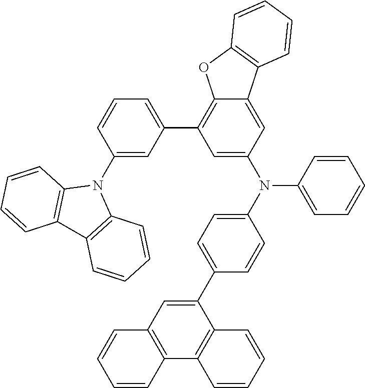 [SMILES: c1ccc(N(c2ccc(-c3cc4ccccc4c4ccccc34)cc2)c2cc(-c3cccc(-n4c5ccccc5c5ccccc54)c3)c3oc4ccccc4c3c2)cc1] | 9.7 g (83%) | 752.28 |
| 80 | 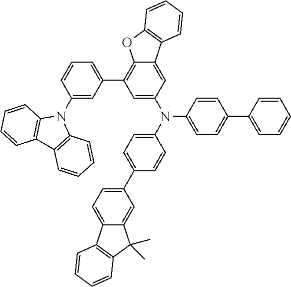 [SMILES: CC1(C)c2ccccc2-c2ccc(-c3ccc(N(c4ccc(-c5ccccc5)cc4)c4cc(-c5cccc(-n6c7ccccc7c7ccccc76)c5)c5oc6ccccc6c5c4)cc3)cc21] | 11.3 g (86%) | 844.35 |
| 81 | 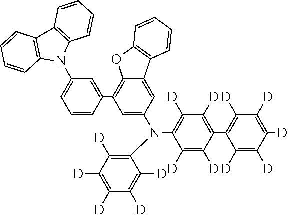 [SMILES: [2H]c1c([2H])c([2H])c(-c2c([2H])c([2H])c(N(c3cc(-c4cccc(-n5c6ccccc6c6ccccc65)c4)c4oc5ccccc5c4c3)c3c([2H])c([2H])c([2H])c([2H])c3[2H])c([2H])c2[2H])c([2H])c1[2H]] | 8.9 g (86%) | 666.34 |
| 82 | 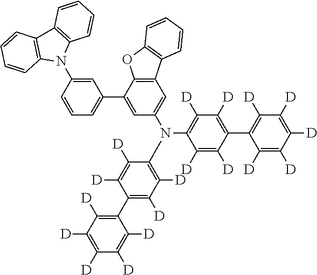 [SMILES: [2H]c1c([2H])c([2H])c(-c2c([2H])c([2H])c(N(c3cc(-c4cccc(-n5c6ccccc6c6ccccc65)c4)c4oc5ccccc5c4c3)c3c([2H])c([2H])c(-c4c([2H])c([2H])c([2H])c([2H])c4[2H])c([2H])c3[2H])c([2H])c2[2H])c([2H])c1[2H]] | 9.1 g (78%) | 746.40 |
| 83 | 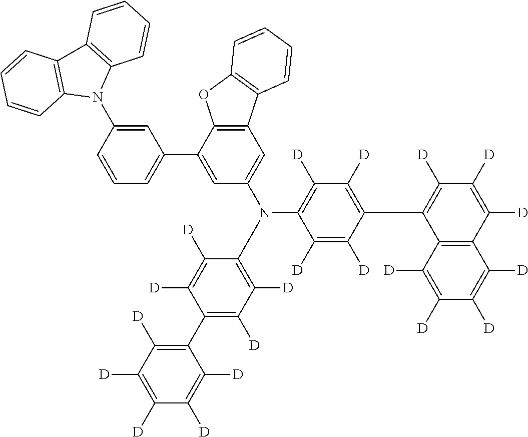 [SMILES: [2H]c1c([2H])c([2H])c(-c2c([2H])c([2H])c(N(c3cc(-c4cccc(-n5c6ccccc6c6ccccc65)c4)c4oc5ccccc5c4c3)c3c([2H])c([2H])c(-c4c([2H])c([2H])c([2H])c5c([2H])c([2H])c([2H])c([2H])c45)c([2H])c3[2H])c([2H])c2[2H])c([2H])c1[2H]] | 9.6 g (77%) | 798.42 |
| 84 | 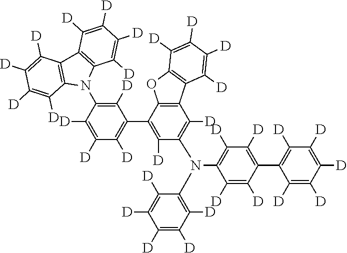 [SMILES: [2H]c1c([2H])c([2H])c(-c2c([2H])c([2H])c(N(c3c([2H])c([2H])c([2H])c([2H])c3[2H])c3c([2H])c(-c4c([2H])c([2H])c([2H])c(-n5c6c([2H])c([2H])c([2H])c([2H])c6c6c([2H])c([2H])c([2H])c([2H])c65)c4[2H])c4oc5c([2H])c([2H])c([2H])c([2H])c5c4c3[2H])c([2H])c2[2H])c([2H])c1[2H]] | 8.7 g (82%) | 684.45 |
| 85 | 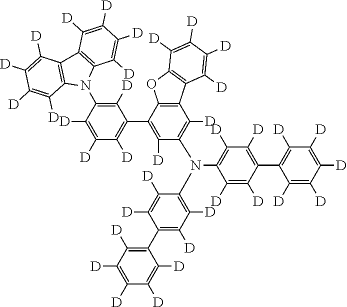 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c(-c4c([2H])c([2H])c([2H])c(-n5c6c([2H])c([2H])c([2H])c([2H])c6c6c([2H])c([2H])c([2H])c([2H])c65)c4[2H])c4oc5c([2H])c([2H])c([2H])c([2H])c5c4c3[2H])c([2H])c2[2H])c([2H])c1[2H]] | 9.9 g (83%) | 764.51 |
| 86 | 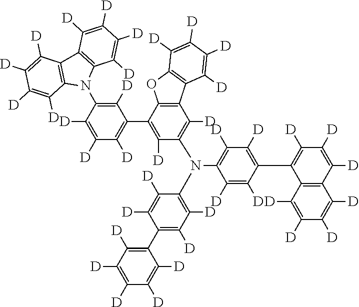 [SMILES: [2H]c1c([2H])c([2H])c(-c2c([2H])c([2H])c(N(c3c([2H])c([2H])c(-c4c([2H])c([2H])c([2H])c5c([2H])c([2H])c([2H])c([2H])c45)c([2H])c3[2H])c3c([2H])c(-c4c([2H])c([2H])c([2H])c(-n5c6c([2H])c([2H])c([2H])c([2H])c6c6c([2H])c([2H])c([2H])c([2H])c65)c4[2H])c4oc5c([2H])c([2H])c([2H])c([2H])c5c4c3[2H])c([2H])c2[2H])c([2H])c1[2H]] | 10.2 g (80%) | 816.54 |

TABLE 9

| | SUB 2-7 | | | |
|---|---|---|---|---|
| Compound | SUB 2-19 | SUB 1 | | |
| 87 | 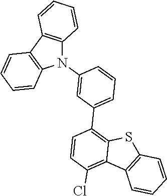 [SMILES: Clc1ccc(-c2cccc(-n3c4ccccc4c4ccccc43)c2)c2sc3ccccc3c12] | 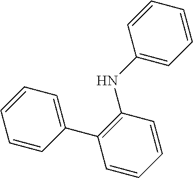 [SMILES: c1ccc(Nc2ccccc2-c2ccccc2)cc1] | | |
| 88 | | 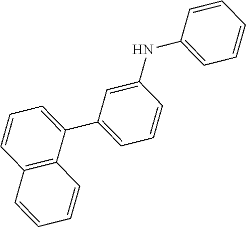 [SMILES: c1ccc(Nc2cccc(-c3cccc4ccccc34)c2)cc1] | | |
| 89 | | 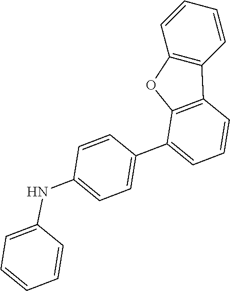 [SMILES: c1ccc(Nc2ccc(-c3cccc4c3oc3ccccc34)cc2)cc1] | | |
| 90 | | 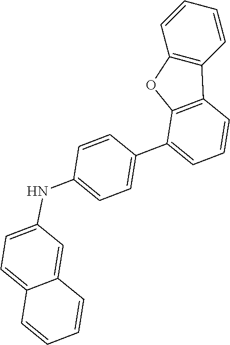 [SMILES: c1ccc2cc(Nc3ccc(-c4cccc5c4oc4ccccc45)cc3)ccc2c1] | | |
| 91 | | 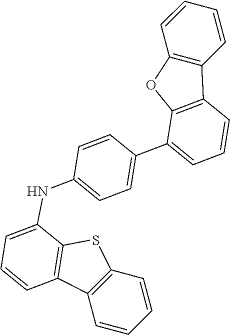 [SMILES: c1ccc2c(c1)oc1c(-c3ccc(Nc4cccc5c4sc4ccccc45)cc3)cccc12] | | |
| 92 | | 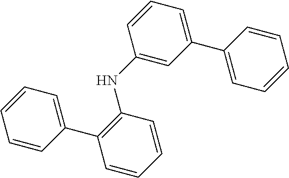 [SMILES: c1ccc(-c2cccc(Nc3ccccc3-c3ccccc3)c2)cc1] | | |
| 93 | | 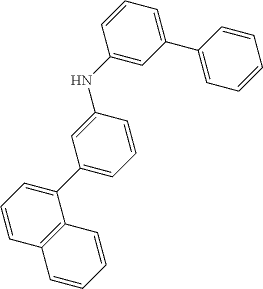 [SMILES: c1ccc(-c2cccc(Nc3cccc(-c4cccc5ccccc45)c3)c2)cc1] | | |
| 94 | | 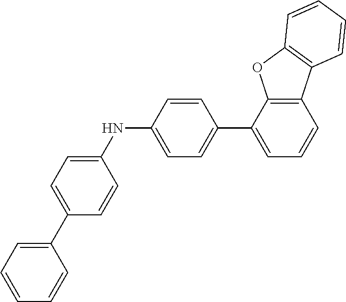 [SMILES: c1ccc(-c2ccc(Nc3ccc(-c4cccc5c4oc4ccccc45)cc3)cc2)cc1] | | |
| 95 | | 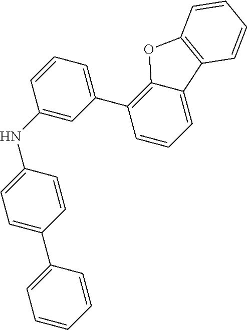 [SMILES: c1ccc(-c2ccc(Nc3cccc(-c4cccc5c4oc4ccccc45)c3)cc2)cc1] | | |
| 96 | | 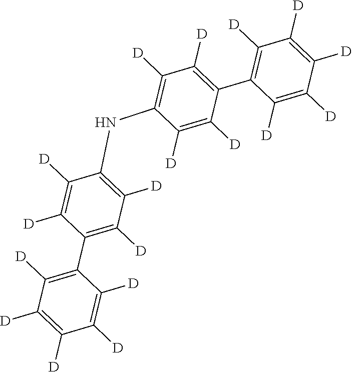 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] | | |
| 97 | | 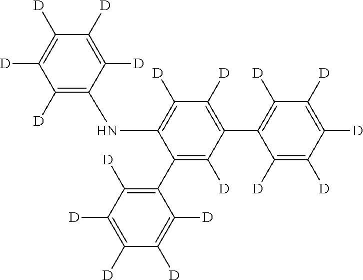 [SMILES: [2H]c1c([2H])c([2H])c(Nc2c([2H])c([2H])c(-c3c([2H])c([2H])c([2H])c([2H])c3[2H])c([2H])c2-c2c([2H])c([2H])c([2H])c([2H])c2[2H])c([2H])c1[2H]] | | |
| 98 | 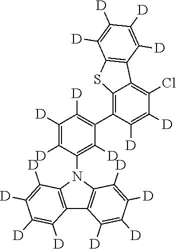 [SMILES: [2H]c1c([2H])c(-c2c([2H])c([2H])c(Cl)c3c2sc2c([2H])c([2H])c([2H])c([2H])c23)c([2H])c(-n2c3c([2H])c([2H])c([2H])c([2H])c3c3c([2H])c([2H])c([2H])c([2H])c32)c1[2H]] | 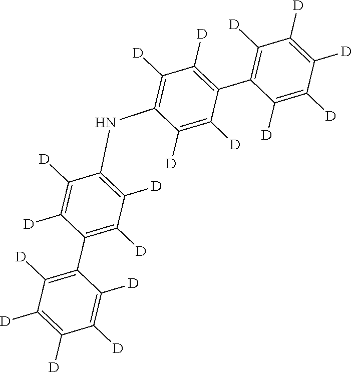 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] | | |
| 99 | | 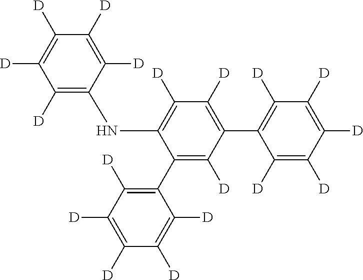 [SMILES: [2H]c1c([2H])c([2H])c(Nc2c([2H])c([2H])c(-c3c([2H])c([2H])c([2H])c([2H])c3[2H])c([2H])c2-c2c([2H])c([2H])c([2H])c([2H])c2[2H])c([2H])c1[2H]] | | |
| | | | Obtained | |
| | | | amount | [M + |
| | Compound | Product | (Yield) | H]⁺ |
| | 87 | 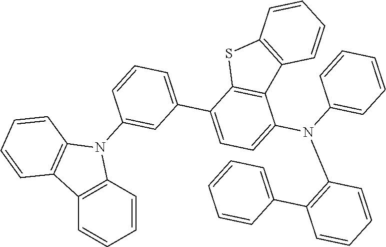 [SMILES: c1ccc(-c2ccccc2N(c2ccccc2)c2ccc(-c3cccc(-n4c5ccccc5c5ccccc54)c3)c3sc4ccccc4c23)cc1] | 8.2 g (79%) | 668.23 |
| | 88 | 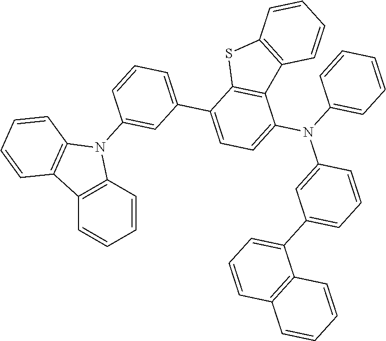 [SMILES: c1ccc(N(c2cccc(-c3cccc4ccccc34)c2)c2ccc(-c3cccc(-n4c5ccccc5c5ccccc54)c3)c3sc4ccccc4c23)cc1] | 8.5 g (76%) | 718.24 |
| | 89 | 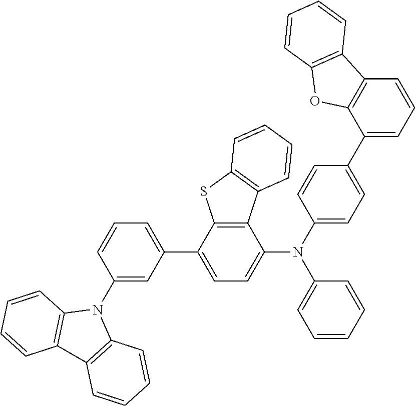 [SMILES: c1ccc(N(c2ccc(-c3cccc4c3oc3ccccc34)cc2)c2ccc(-c3cccc(-n4c5ccccc5c5ccccc54)c3)c3sc4ccccc4c23)cc1] | 9.8 g (83%) | 758.24 |
| | 90 | 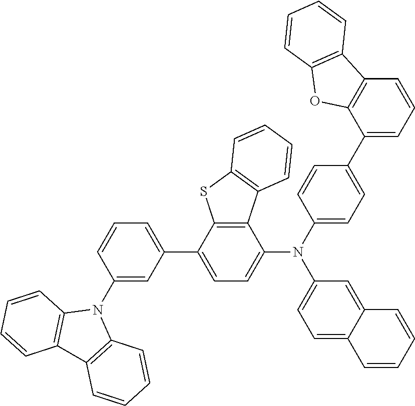 [SMILES: c1cc(-c2ccc(N(c3ccc(-c4cccc5c4oc4ccccc45)cc3)c3ccc4ccccc4c3)c3c2sc2ccccc23)cc(-n2c3ccccc3c3ccccc32)c1] | 9.1 g (72%) | 808.25 |
| | 91 | 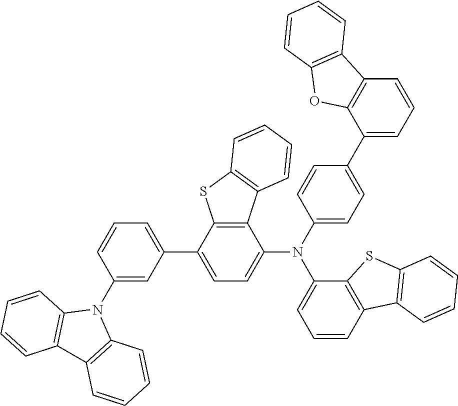 [SMILES: c1cc(-c2ccc(N(c3ccc(-c4cccc5c4oc4ccccc45)cc3)c3cccc4c3sc3ccccc34)c3c2sc2ccccc23)cc(-n2c3ccccc3c3ccccc32)c1] | 10.9 g (81%) | 864.23 |
| | 92 | 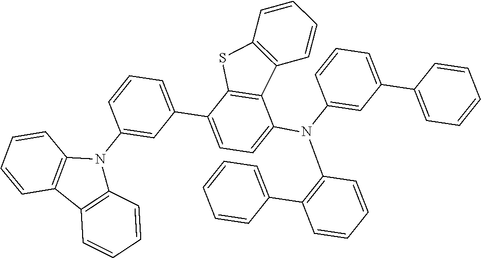 [SMILES: c1ccc(-c2cccc(N(c3ccccc3-c3ccccc3)c3ccc(-c4cccc(-n5c6ccccc6c6ccccc65)c4)c4sc5ccccc5c34)c2)cc1] | 9.6 g (83%) | 744.26 |
| | 93 | 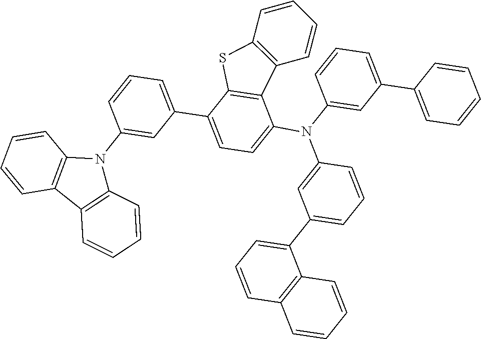 [SMILES: c1ccc(-c2cccc(N(c3cccc(-c4cccc5ccccc45)c3)c3ccc(-c4cccc(-n5c6ccccc6c6ccccc65)c4)c4sc5ccccc5c34)c2)cc1] | 9.8 g (79%) | 794.28 |
| | 94 | 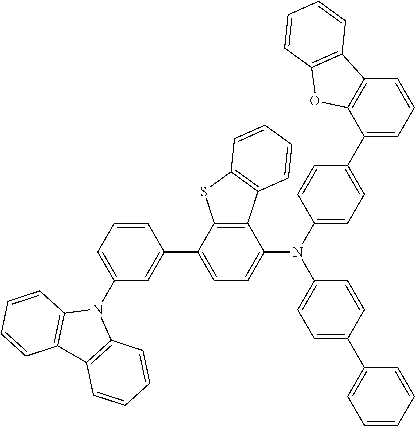 [SMILES: c1ccc(-c2ccc(N(c3ccc(-c4cccc5c4oc4ccccc45)cc3)c3ccc(-c4cccc(-n5c6ccccc6c6ccccc65)c4)c4sc5ccccc5c34)cc2)cc1] | 11.2 g (86%) | 834.27 |
| | 95 | 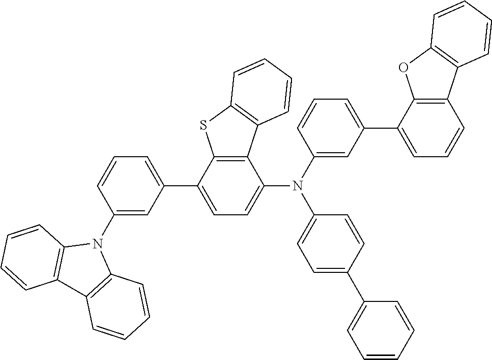 [SMILES: c1ccc(-c2ccc(N(c3cccc(-c4cccc5c4oc4ccccc45)c3)c3ccc(-c4cccc(-n5c6ccccc6c6ccccc65)c4)c4sc5ccccc5c34)cc2)cc1] | 11.4 g (88%) | 834.27 |
| | 96 | 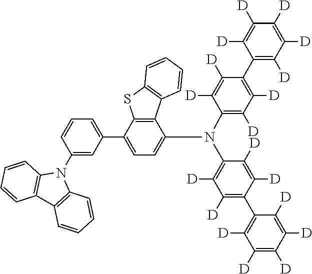 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3ccc(-c4cccc(-n5c6ccccc6c6ccccc65)c4)c4sc5ccccc5c34)c([2H])c2[2H])c([2H])c1[2H]] | 10.2 g (86%) | 762.37 |
| | 97 | 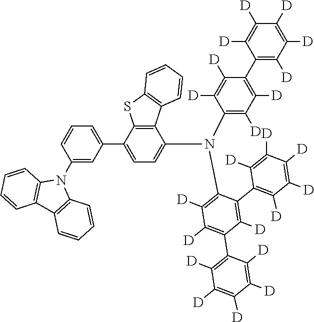 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3-c3c([2H])c([2H])c([2H])c([2H])c3[2H])c3ccc(-c4cccc(-n5c6ccccc6c6ccccc65)c4)c4sc5ccccc5c34)c([2H])c2[2H])c([2H])c1[2H]] | 11.0 g (84%) | 842.43 |
| | 98 | 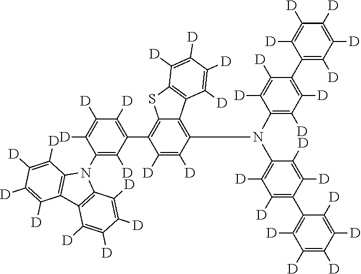 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c(-c4c([2H])c([2H])c([2H])c(-n5c6c([2H])c([2H])c([2H])c([2H])c6c6c([2H])c([2H])c([2H])c([2H])c65)c4[2H])c4sc5c([2H])c([2H])c([2H])c([2H])c5c34)c([2H])c2[2H])c([2H])c1[2H]] | 9.2 g (76%) | 780.49 |
| | 99 | 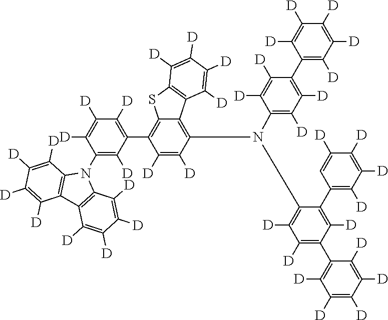 [SMILES: [2H]c1cc(-c2c([2H])c(-c3c([2H])c([2H])c([2H])c([2H])c3[2H])c([2H])c([2H])c2N(c2c([2H])c([2H])c(-c3c([2H])c([2H])c([2H])c([2H])c3[2H])c([2H])c2[2H])c2c([2H])c([2H])c(-c3c([2H])c([2H])c([2H])c(-n4c5c([2H])c([2H])c([2H])c([2H])c5c5c([2H])c([2H])c([2H])c([2H])c54)c3[2H])c3sc4c([2H])c([2H])c([2H])c([2H])c4c23)c([2H])c([2H])c1[2H]] | 10.3 g (77%) | 860.54 |

TABLE 10

| | SUB 2-8 | | | |
|---|---|---|---|---|
| Compound | SUB 2-20 | SUB 1 | | |
| 100 | 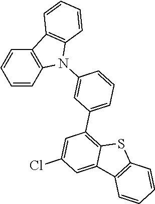 [SMILES: Clc1cc(-c2cccc(-n3c4ccccc4c4ccccc43)c2)c2sc3ccccc3c2c1] | 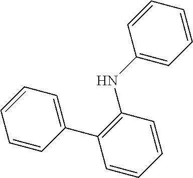 [SMILES: c1ccc(Nc2ccccc2-c2ccccc2)cc1] | | |
| 101 | | 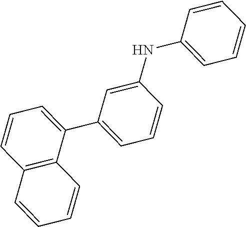 [SMILES: c1ccc(Nc2cccc(-c3cccc4ccccc34)c2)cc1] | | |
| 102 | | 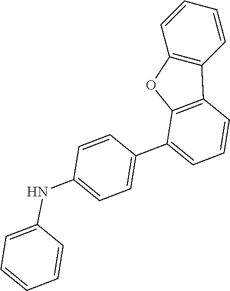 [SMILES: c1ccc(Nc2ccc(-c3cccc4c3oc3ccccc34)cc2)cc1] | | |
| 103 | | 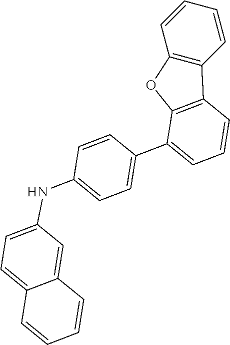 [SMILES: c1ccc2cc(Nc3ccc(-c4cccc5c4oc4ccccc45)cc3)ccc2c1] | | |
| 104 | | 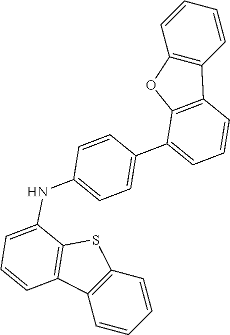 [SMILES: c1ccc2c(c1)oc1c(-c3ccc(Nc4cccc5c4sc4ccccc45)cc3)cccc12] | | |
| 105 | | 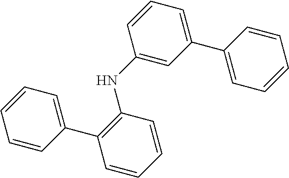 [SMILES: c1ccc(-c2cccc(Nc3ccccc3-c3ccccc3)c2)cc1] | | |
| 106 | | 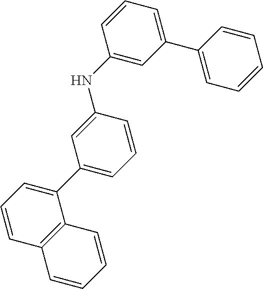 [SMILES: c1ccc(-c2cccc(Nc3cccc(-c4cccc5ccccc45)c3)c2)cc1] | | |
| 107 | | 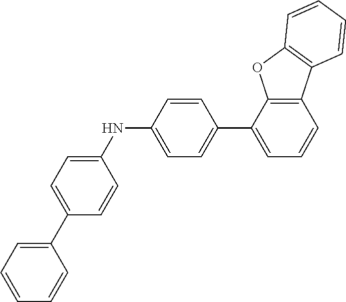 [SMILES: c1ccc(-c2ccc(Nc3ccc(-c4cccc5c4oc4ccccc45)cc3)cc2)cc1] | | |
| 108 | | 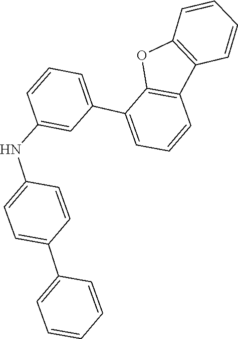 [SMILES: c1ccc(-c2ccc(Nc3cccc(-c4cccc5c4oc4ccccc45)c3)cc2)cc1] | | |
| 109 | | 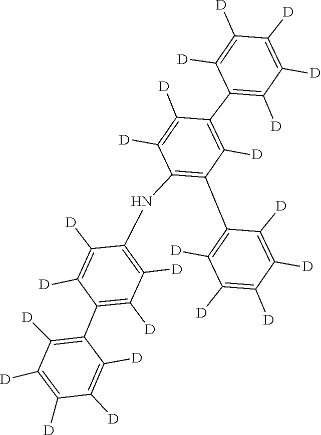 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3-c3c([2H])c([2H])c([2H])c([2H])c3[2H])c([2H])c2[2H])c([2H])c1[2H]] | | |
| 110 | | 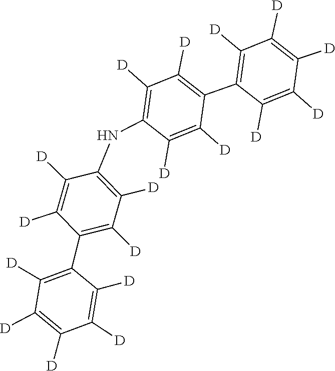 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] | | |
| 111 | 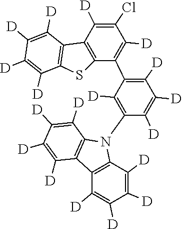 [SMILES: [2H]c1c([2H])c(-c2c([2H])c(Cl)c([2H])c3c2sc2c([2H])c([2H])c([2H])c([2H])c23)c([2H])c(-n2c3c([2H])c([2H])c([2H])c([2H])c3c3c([2H])c([2H])c([2H])c([2H])c32)c1[2H]] | 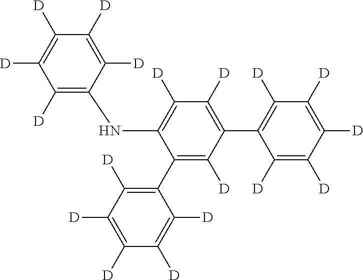 [SMILES: [2H]c1c([2H])c([2H])c(Nc2c([2H])c([2H])c(-c3c([2H])c([2H])c([2H])c([2H])c3[2H])c([2H])c2-c2c([2H])c([2H])c([2H])c([2H])c2[2H])c([2H])c1[2H]] | | |
| 112 | | 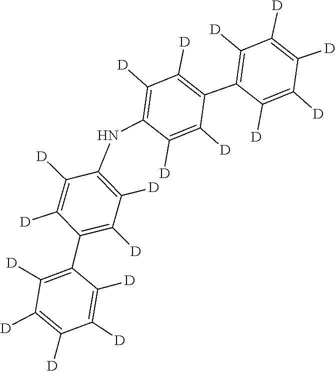 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] | | |
| | | | Obtained | |
| | | | amount | |
| | Compound | Product | (Yield) | [M + H]⁺ |
| | 100 | 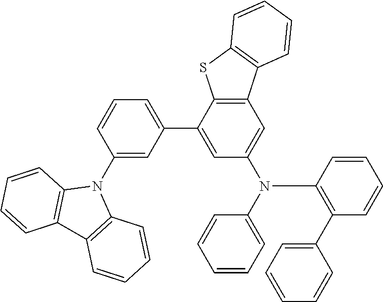 [SMILES: c1ccc(-c2ccccc2N(c2ccccc2)c2cc(-c3cccc(-n4c5ccccc5c5ccccc54)c3)c3sc4ccccc4c3c2)cc1] | 7.7 g (74%) | 668.23 |
| | 101 | 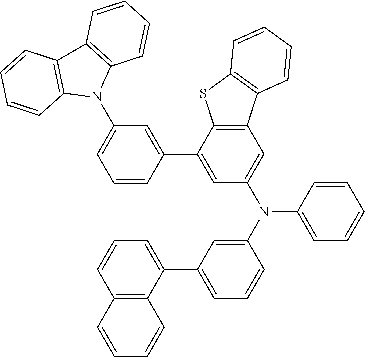 [SMILES: c1ccc(N(c2cccc(-c3cccc4ccccc34)c2)c2cc(-c3cccc(-n4c5ccccc5c5ccccc54)c3)c3sc4ccccc4c3c2)cc1] | 8.6 g (77%) | 718.24 |
| | 102 | 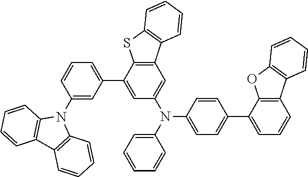 [SMILES: c1ccc(N(c2ccc(-c3cccc4c3oc3ccccc34)cc2)c2cc(-c3cccc(-n4c5ccccc5c5ccccc54)c3)c3sc4ccccc4c3c2)cc1] | 9.3 g (79%) | 758.24 |
| | 103 | 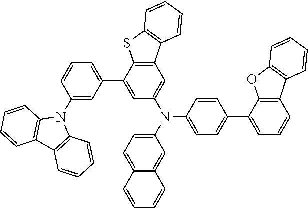 [SMILES: c1cc(-c2cc(N(c3ccc(-c4cccc5c4oc4ccccc45)cc3)c3ccc4ccccc4c3)cc3c2sc2ccccc23)cc(-n2c3ccccc3c3ccccc32)c1] | 10.2 g (81%) | 808.25 |
| | 104 | 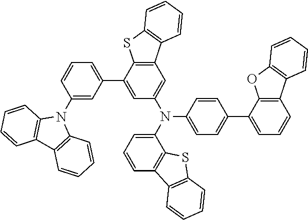 [SMILES: c1cc(-c2cc(N(c3ccc(-c4cccc5c4oc4ccccc45)cc3)c3cccc4c3sc3ccccc34)cc3c2sc2ccccc23)cc(-n2c3ccccc3c3ccccc32)c1] | 10.2 g (76%) | 864.23 |
| | 105 | 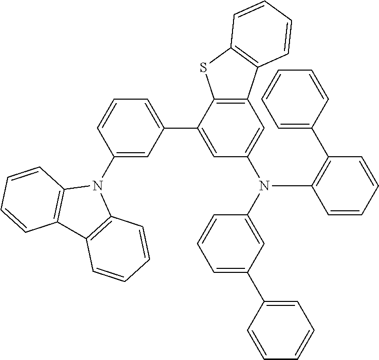 [SMILES: c1ccc(-c2cccc(N(c3cc(-c4cccc(-n5c6ccccc6c6ccccc65)c4)c4sc5ccccc5c4c3)c3ccccc3-c3ccccc3)c2)cc1] | 9.3 g (80%) | 744.26 |
| | 106 | 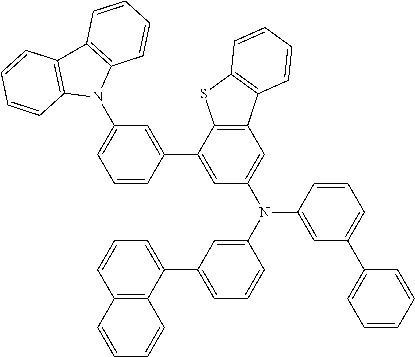 [SMILES: c1ccc(-c2cccc(N(c3cccc(-c4cccc5ccccc45)c3)c3cc(-c4cccc(-n5c6ccccc6c6ccccc65)c4)c4sc5ccccc5c4c3)c2)cc1] | 9.4 g (76%) | 794.28 |
| | 107 | 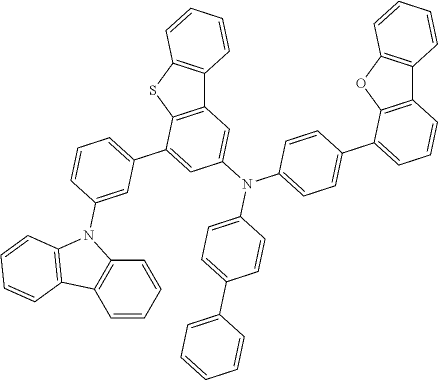 [SMILES: c1ccc(-c2ccc(N(c3ccc(-c4cccc5c4oc4ccccc45)cc3)c3cc(-c4cccc(-n5c6ccccc6c6ccccc65)c4)c4sc5ccccc5c4c3)cc2)cc1] | 9.6 g (74%) | 834.27 |
| | 108 | 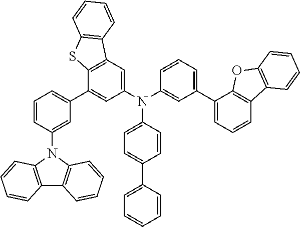 [SMILES: c1ccc(-c2ccc(N(c3cccc(-c4cccc5c4oc4ccccc45)c3)c3cc(-c4cccc(-n5c6ccccc6c6ccccc65)c4)c4sc5ccccc5c4c3)cc2)cc1] | 9.5 g (73%) | 834.27 |
| | 109 | 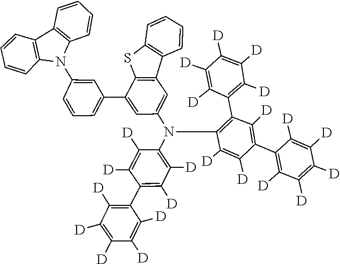 [SMILES: [2H]c1c([2H])c([2H])c(-c2c([2H])c([2H])c(N(c3cc(-c4cccc(-n5c6ccccc6c6ccccc65)c4)c4sc5ccccc5c4c3)c3c([2H])c([2H])c(-c4c([2H])c([2H])c([2H])c([2H])c4[2H])c([2H])c3-c3c([2H])c([2H])c([2H])c([2H])c3[2H])c([2H])c2[2H])c([2H])c1[2H]] | 9.7 g (72%) | 826.45 |
| | 110 | 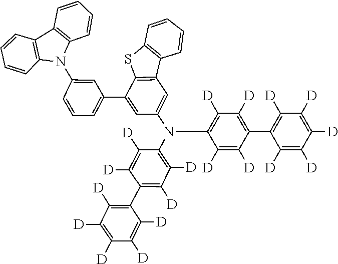 [SMILES: [2H]c1c([2H])c([2H])c(-c2c([2H])c([2H])c(N(c3cc(-c4cccc(-n5c6ccccc6c6ccccc65)c4)c4sc5ccccc5c4c3)c3c([2H])c([2H])c(-c4c([2H])c([2H])c([2H])c([2H])c4[2H])c([2H])c3[2H])c([2H])c2[2H])c([2H])c1[2H]] | 9.3 g (78%) | 762.37 |
| | 111 | 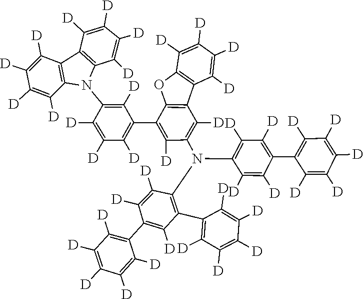 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3-c3c([2H])c([2H])c([2H])c([2H])c3[2H])c3c([2H])c(-c4c([2H])c([2H])c([2H])c(-n5c6c([2H])c([2H])c([2H])c([2H])c6c6c([2H])c([2H])c([2H])c([2H])c65)c4[2H])c4oc5c([2H])c([2H])c([2H])c([2H])c5c4c3[2H])c([2H])c2[2H])c([2H])c1[2H]] | 10.0 g (76%) | 844.57 |
| | 112 | 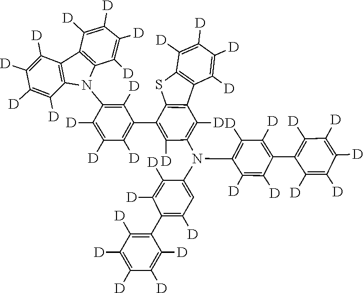 [SMILES: [2H]c1cc(N(c2c([2H])c([2H])c(-c3c([2H])c([2H])c([2H])c([2H])c3[2H])c([2H])c2[2H])c2c([2H])c(-c3c([2H])c([2H])c([2H])c(-n4c5c([2H])c([2H])c([2H])c([2H])c5c5c([2H])c([2H])c([2H])c([2H])c54)c3[2H])c3sc4c([2H])c([2H])c([2H])c([2H])c4c3c2[2H])c([2H])c([2H])c1-c1c([2H])c([2H])c([2H])c([2H])c1[2H]] | 9.7 g (80%) | 780.49 |

TABLE 11

| | SUB 2-9 | | |
|---|---|---|---|
| Compound | SUB 2-21 | SUB 1 | |
| 113 | 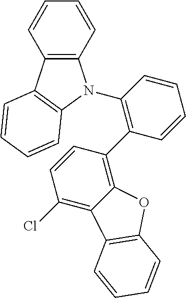 [SMILES: Clc1ccc(-c2ccccc2-n2c3ccccc3c3ccccc32)c2oc3ccccc3c12] | 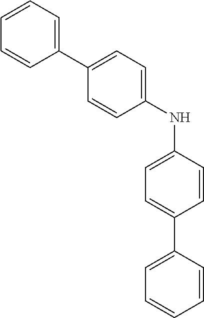 [SMILES: c1ccc(-c2ccc(Nc3ccc(-c4ccccc4)cc3)cc2)cc1] | |
| 114 | | 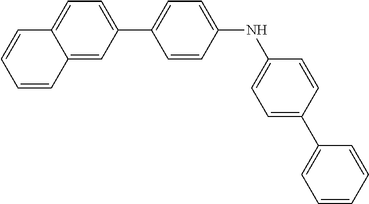 [SMILES: c1ccc(-c2ccc(Nc3ccc(-c4ccc5ccccc5c4)cc3)cc2)cc1] | |
| 115 | | 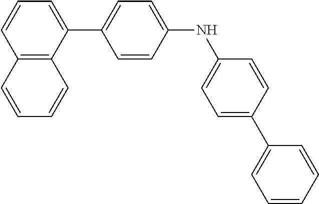 [SMILES: c1ccc(-c2ccc(Nc3ccc(-c4cccc5ccccc45)cc3)cc2)cc1] | |
| 116 | | 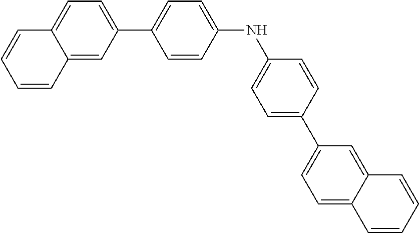 [SMILES: c1ccc2cc(-c3ccc(Nc4ccc(-c5ccc6ccccc6c5)cc4)cc3)ccc2c1] | |
| 117 | | 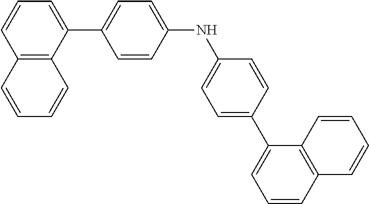 [SMILES: c1ccc2c(-c3ccc(Nc4ccc(-c5cccc6ccccc56)cc4)cc3)cccc2c1] | |
| 118 | | 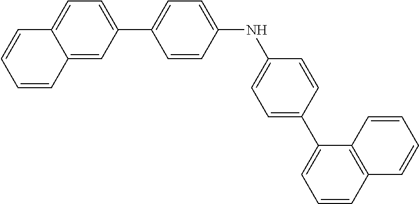 [SMILES: c1ccc2cc(-c3ccc(Nc4ccc(-c5cccc6ccccc56)cc4)cc3)ccc2c1] | |
| 119 | | 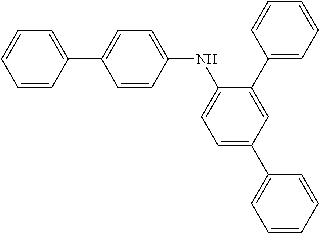 [SMILES: c1ccc(-c2ccc(Nc3ccc(-c4ccccc4)cc3-c3ccccc3)cc2)cc1] | |
| 120 | | 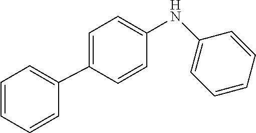 [SMILES: c1ccc(Nc2ccc(-c3ccccc3)cc2)cc1] | |
| 121 | | 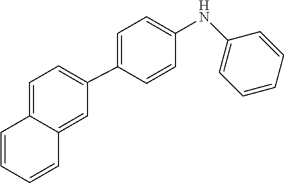 [SMILES: c1ccc(Nc2ccc(-c3ccc4ccccc4c3)cc2)cc1] | |
| 122 | | 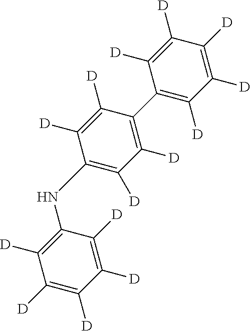 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] | |
| 123 | | 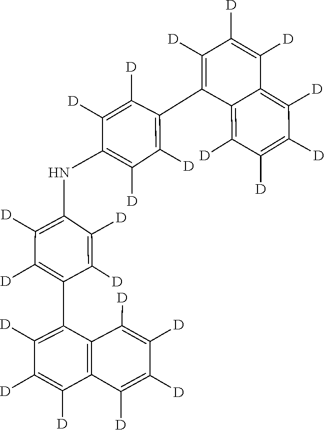 [SMILES: [2H]c1c([2H])c(-c2c([2H])c([2H])c([2H])c3c([2H])c([2H])c([2H])c([2H])c23)c([2H])c([2H])c1Nc1c([2H])c([2H])c(-c2c([2H])c([2H])c([2H])c3c([2H])c([2H])c([2H])c([2H])c23)c([2H])c1[2H]] | |
| 124 | | 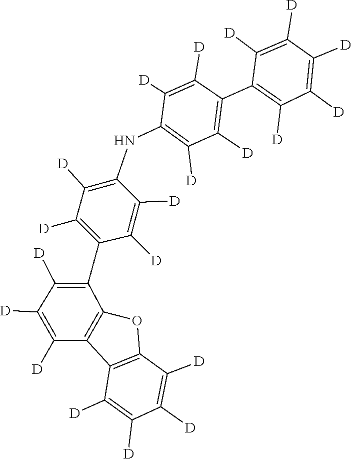 [SMILES: [2H]c1c([2H])c([2H])c(-c2c([2H])c([2H])c(Nc3c([2H])c([2H])c(-c4c([2H])c([2H])c([2H])c5c4oc4c([2H])c([2H])c([2H])c([2H])c45)c([2H])c3[2H])c([2H])c2[2H])c([2H])c1[2H]] | |
| 125 | 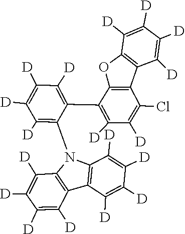 [SMILES: [2H]c1c([2H])c([2H])c(-n2c3c([2H])c([2H])c([2H])c([2H])c3c3c([2H])c([2H])c([2H])c([2H])c32)c(-c2c([2H])c([2H])c(Cl)c3c2oc2c([2H])c([2H])c([2H])c([2H])c23)c1[2H]] | 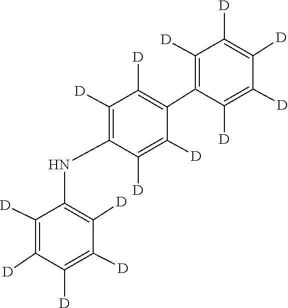 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] | |
| 126 | | 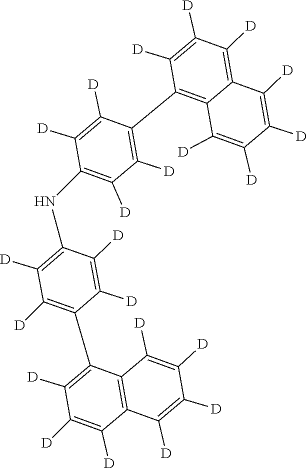 [SMILES: [2H]c1c([2H])c(-c2c([2H])c([2H])c([2H])c3c([2H])c([2H])c([2H])c([2H])c23)c([2H])c([2H])c1Nc1c([2H])c([2H])c(-c2c([2H])c([2H])c([2H])c3c([2H])c([2H])c([2H])c([2H])c23)c([2H])c1[2H]] | |
| 127 | | 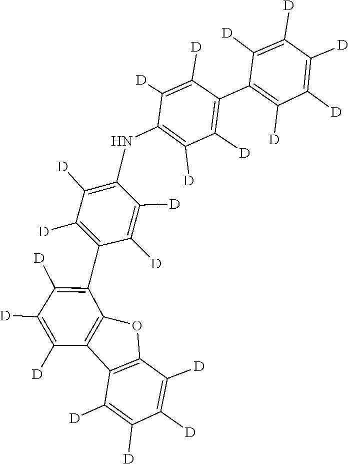 [SMILES: [2H]c1c([2H])c([2H])c(-c2c([2H])c([2H])c(Nc3c([2H])c([2H])c(-c4c([2H])c([2H])c([2H])c5c4oc4c([2H])c([2H])c([2H])c([2H])c45)c([2H])c3[2H])c([2H])c2[2H])c([2H])c1[2H]] | |
| | | | Obtained |
| | | | amount |
| | Compound | Product | (Yield) |
| | 113 | 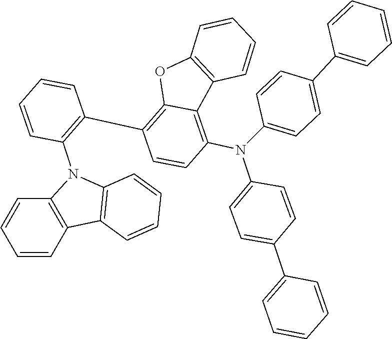 [SMILES: c1ccc(-c2ccc(N(c3ccc(-c4ccccc4)cc3)c3ccc(-c4ccccc4-n4c5ccccc5c5ccccc54)c4oc5ccccc5c34)cc2)cc1] | 9.9 g (87%) |
| | 114 | 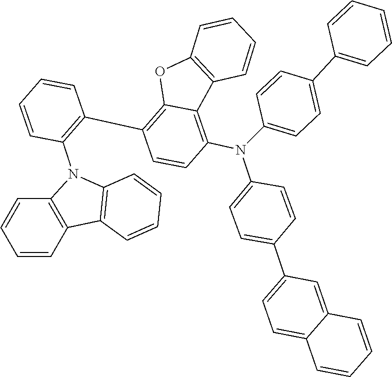 [SMILES: c1ccc(-c2ccc(N(c3ccc(-c4ccc5ccccc5c4)cc3)c3ccc(-c4ccccc4-n4c5ccccc5c5ccccc54)c4oc5ccccc5c34)cc2)cc1] | 9.8 g (81%) |
| | 115 | 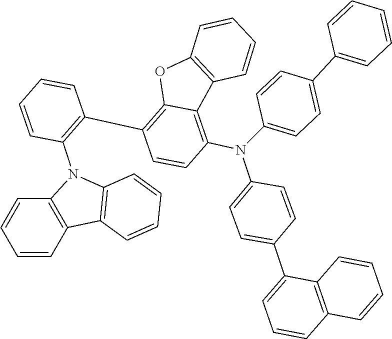 [SMILES: c1ccc(-c2ccc(N(c3ccc(-c4cccc5ccccc45)cc3)c3ccc(-c4ccccc4-n4c5ccccc5c5ccccc54)c4oc5ccccc5c34)cc2)cc1] | 10.1 g (83%) |
| | 116 | 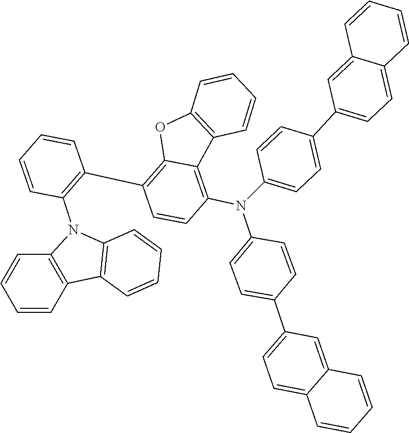 [SMILES: c1ccc(-n2c3ccccc3c3ccccc32)c(-c2ccc(N(c3ccc(-c4ccc5ccccc5c4)cc3)c3ccc(-c4ccc5ccccc5c4)cc3)c3c2oc2ccccc23)c1] | 11.1 g (86%) |
| | 117 | 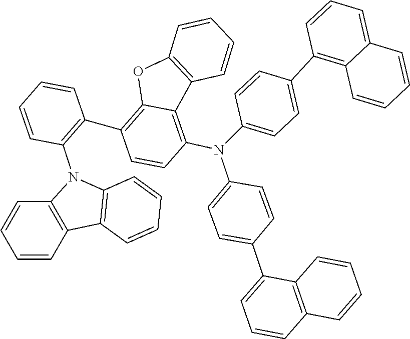 [SMILES: c1ccc(-n2c3ccccc3c3ccccc32)c(-c2ccc(N(c3ccc(-c4cccc5ccccc45)cc3)c3ccc(-c4cccc5ccccc45)cc3)c3c2oc2ccccc23)c1] | 11.0 g (85%) |
| | 118 | 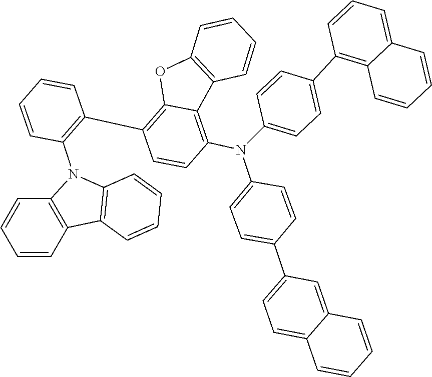 [SMILES: c1ccc(-n2c3ccccc3c3ccccc32)c(-c2ccc(N(c3ccc(-c4ccc5ccccc5c4)cc3)c3ccc(-c4cccc5ccccc45)cc3)c3c2oc2ccccc23)c1] | 11.1 g (86%) |
| | 119 | 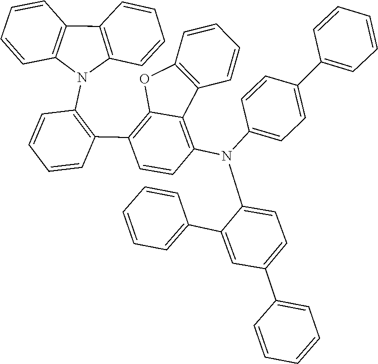 [SMILES: c1ccc(-c2ccc(N(c3ccc(-c4ccccc4)cc3-c3ccccc3)c3ccc(-c4ccccc4-n4c5ccccc5c5ccccc54)c4oc5ccccc5c34)cc2)cc1] | 10.8 g (86%) |
| | 120 | 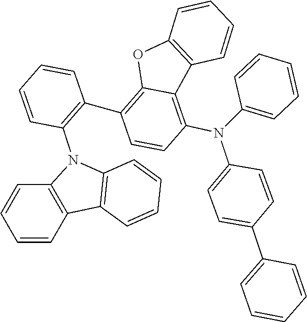 [SMILES: c1ccc(-c2ccc(N(c3ccccc3)c3ccc(-c4ccccc4-n4c5ccccc5c5ccccc54)c4oc5ccccc5c34)cc2)cc1] | 8.3 g (82%) |
| | 121 | 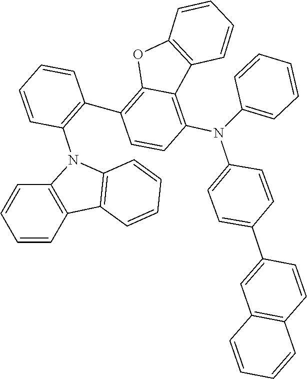 [SMILES: c1ccc(N(c2ccc(-c3ccc4ccccc4c3)cc2)c2ccc(-c3ccccc3-n3c4ccccc4c4ccccc43)c3oc4ccccc4c23)cc1] | 8.6 g (79%) |
| | 122 | 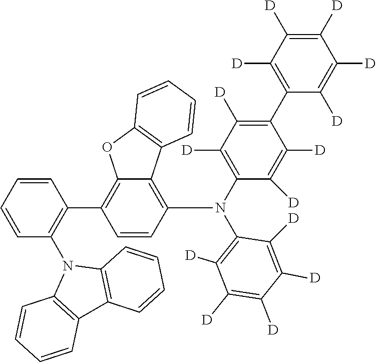 [SMILES: [2H]c1c([2H])c([2H])c(-c2c([2H])c([2H])c(N(c3c([2H])c([2H])c([2H])c([2H])c3[2H])c3ccc(-c4ccccc4-n4c5ccccc5c5ccccc54)c4oc5ccccc5c34)c([2H])c2[2H])c([2H])c1[2H]] | 8.2 g (79%) |
| | 123 | 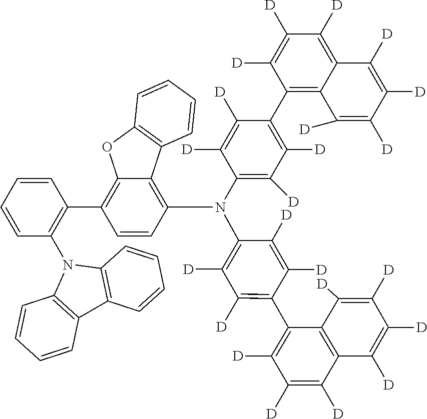 [SMILES: [2H]c1c([2H])c(N(c2c([2H])c([2H])c(-c3c([2H])c([2H])c([2H])c4c([2H])c([2H])c([2H])c([2H])c34)c([2H])c2[2H])c2ccc(-c3ccccc3-n3c4ccccc4c4ccccc43)c3oc4ccccc4c23)c([2H])c([2H])c1-c1c([2H])c([2H])c([2H])c2c([2H])c([2H])c([2H])c([2H])c12] | 10.3 g (78%) |
| | 124 | 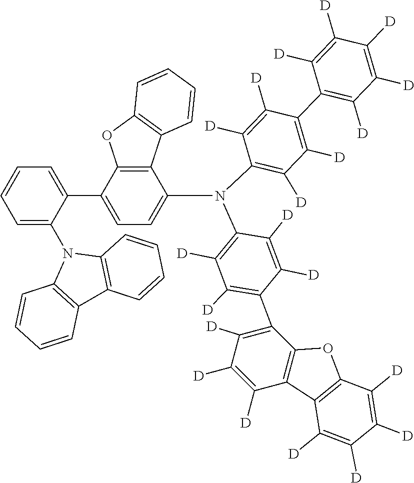 [SMILES: [2H]c1c([2H])c([2H])c(-c2c([2H])c([2H])c(N(c3c([2H])c([2H])c(-c4c([2H])c([2H])c([2H])c5c4oc4c([2H])c([2H])c([2H])c([2H])c45)c([2H])c3[2H])c3ccc(-c4ccccc4-n4c5ccccc5c5ccccc54)c4oc5ccccc5c34)c([2H])c2[2H])c([2H])c1[2H]] | 10.0 g (77%) |
| | 125 | 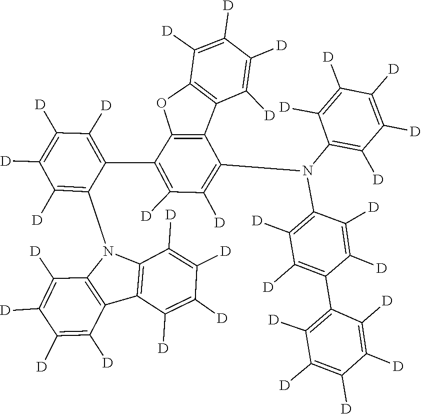 [SMILES: [2H]c1c([2H])c([2H])c(-c2c([2H])c([2H])c(N(c3c([2H])c([2H])c([2H])c([2H])c3[2H])c3c([2H])c([2H])c(-c4c([2H])c([2H])c([2H])c([2H])c4-n4c5c([2H])c([2H])c([2H])c([2H])c5c5c([2H])c([2H])c([2H])c([2H])c54)c4oc5c([2H])c([2H])c([2H])c([2H])c5c34)c([2H])c2[2H])c([2H])c1[2H]] | 8.4 g (79%) |
| | 126 | 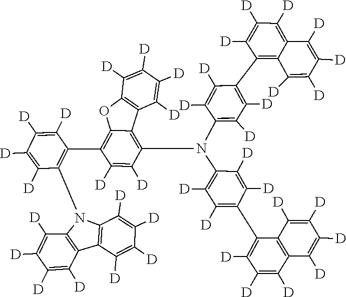 [SMILES: [2H]c1c([2H])c([2H])c(-n2c3c([2H])c([2H])c([2H])c([2H])c3c3c([2H])c([2H])c([2H])c([2H])c32)c(-c2c([2H])c([2H])c(N(c3c([2H])c([2H])c(-c4c([2H])c([2H])c([2H])c5c([2H])c([2H])c([2H])c([2H])c45)c([2H])c3[2H])c3c([2H])c([2H])c(-c4c([2H])c([2H])c([2H])c5c([2H])c([2H])c([2H])c([2H])c45)c([2H])c3[2H])c3c2oc2c([2H])c([2H])c([2H])c([2H])c23)c1[2H]] | 11.2 g (83%) |
| | 127 | 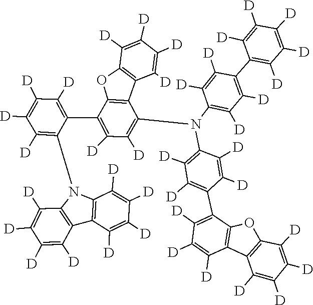 [SMILES: [2H]c1c([2H])c([2H])c(-c2c([2H])c([2H])c(N(c3c([2H])c([2H])c(-c4c([2H])c([2H])c([2H])c5c4oc4c([2H])c([2H])c([2H])c([2H])c45)c([2H])c3[2H])c3c([2H])c([2H])c(-c4c([2H])c([2H])c([2H])c([2H])c4-n4c5c([2H])c([2H])c([2H])c([2H])c5c5c([2H])c([2H])c([2H])c([2H])c54)c4oc5c([2H])c([2H])c([2H])c([2H])c5c34)c([2H])c2[2H])c([2H])c1[2H]] | 10.7 g (80%) |

TABLE 12

| | SUB 2-10 | | | |
|---|---|---|---|---|
| Compound | SUB 2-22 | SUB 1 | | |
| 128 | 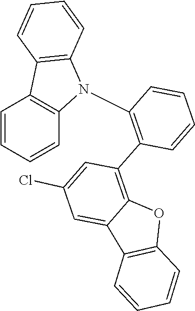 [SMILES: Clc1cc(-c2ccccc2-n2c3ccccc3c3ccccc32)c2oc3ccccc3c2c1] | 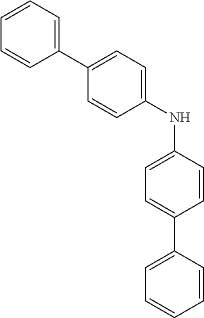 [SMILES: c1ccc(-c2ccc(Nc3ccc(-c4ccccc4)cc3)cc2)cc1] | | |
| 129 | | 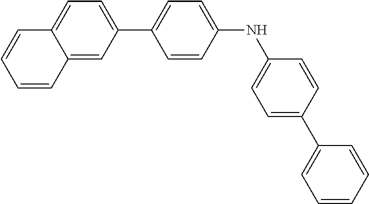 [SMILES: c1ccc(-c2ccc(Nc3ccc(-c4ccc5ccccc5c4)cc3)cc2)cc1] | | |
| 130 | | 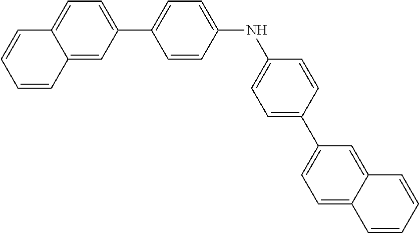 [SMILES: c1ccc2cc(-c3ccc(Nc4ccc(-c5ccc6ccccc6c5)cc4)cc3)ccc2c1] | | |
| 131 | | 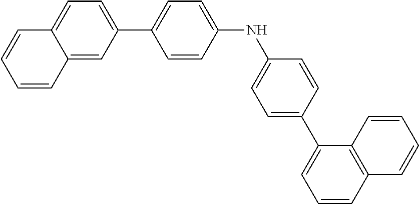 [SMILES: c1ccc2cc(-c3ccc(Nc4ccc(-c5cccc6ccccc56)cc4)cc3)ccc2c1] | | |
| 132 | | 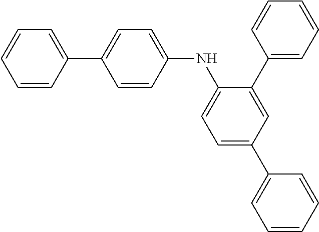 [SMILES: c1ccc(-c2ccc(Nc3ccc(-c4ccccc4)cc3-c3ccccc3)cc2)cc1] | | |
| 133 | | 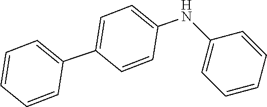 [SMILES: c1ccc(Nc2ccc(-c3ccccc3)cc2)cc1] | | |
| 134 | | 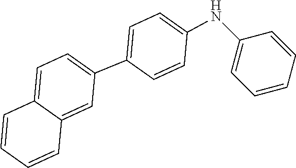 [SMILES: c1ccc(Nc2ccc(-c3ccc4ccccc4c3)cc2)cc1] | | |
| 135 | | 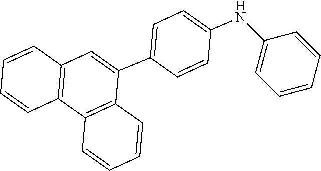 [SMILES: c1ccc(Nc2ccc(-c3cc4ccccc4c4ccccc34)cc2)cc1] | | |
| 136 | | 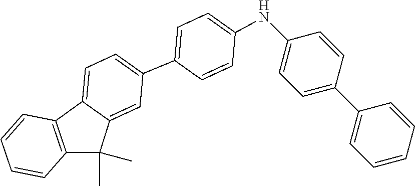 [SMILES: CC1(C)c2ccccc2-c2ccc(-c3ccc(Nc4ccc(-c5ccccc5)cc4)cc3)cc21] | | |
| 137 | | 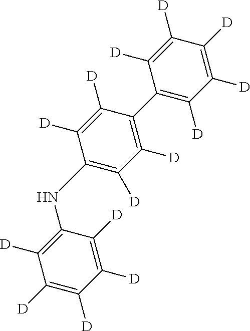 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] | | |
| 138 | | 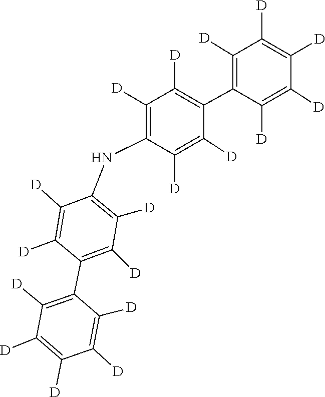 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] | | |
| 139 | | 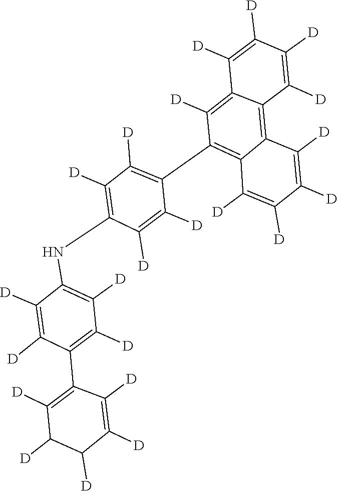 [SMILES: [2H]C1=C([2H])C([2H])C([2H])C([2H])=C1c1c([2H])c([2H])c(Nc2c([2H])c([2H])c(-c3c([2H])c4c([2H])c([2H])c([2H])c([2H])c4c4c([2H])c([2H])c([2H])c([2H])c34)c([2H])c2[2H])c([2H])c1[2H]] | | |
| 140 | 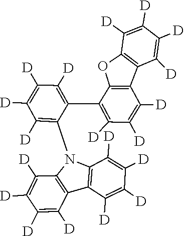 [SMILES: [2H]c1c([2H])c([2H])c(-n2c3c([2H])c([2H])c([2H])c([2H])c3c3c([2H])c([2H])c([2H])c([2H])c32)c(-c2c([2H])c([2H])c([2H])c3c2oc2c([2H])c([2H])c([2H])c([2H])c23)c1[2H]] | 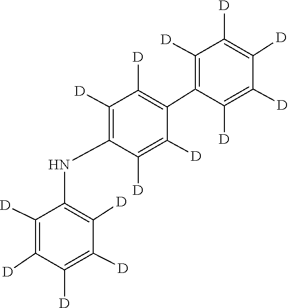 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] | | |
| 141 | | 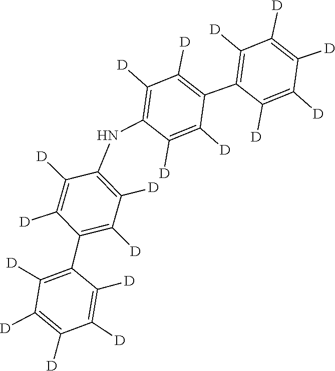 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] | | |
| 142 | | 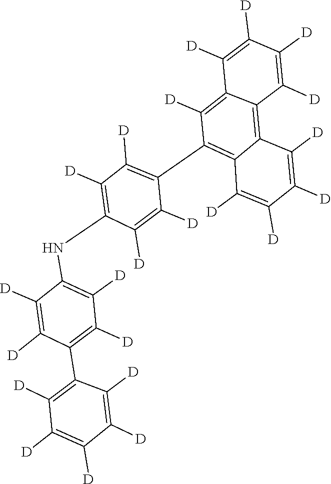 [SMILES: [2H]c1c([2H])c([2H])c(-c2c([2H])c([2H])c(Nc3c([2H])c([2H])c(-c4c([2H])c5c([2H])c([2H])c([2H])c([2H])c5c5c([2H])c([2H])c([2H])c([2H])c45)c([2H])c3[2H])c([2H])c2[2H])c([2H])c1[2H]] | | |
| | | | Obtained | |
| | | | amount | |
| | Compound | Product | (Yield) | [M + H]⁺ |
| | 128 | 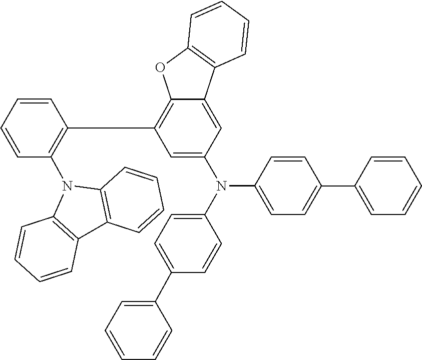 [SMILES: c1ccc(-c2ccc(N(c3ccc(-c4ccccc4)cc3)c3cc(-c4ccccc4-n4c5ccccc5c5ccccc54)c4oc5ccccc5c4c3)cc2)cc1] | 9.2 g (81%) | 728.28 |
| | 129 | 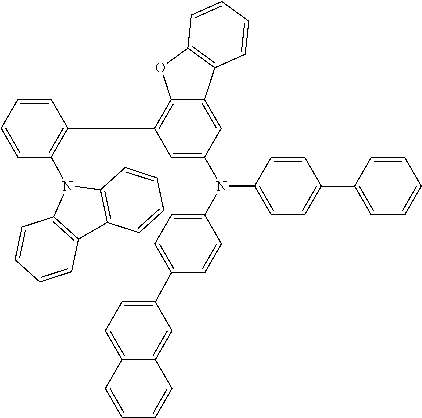 [SMILES: c1ccc(-c2ccc(N(c3ccc(-c4ccc5ccccc5c4)cc3)c3cc(-c4ccccc4-n4c5ccccc5c5ccccc54)c4oc5ccccc5c4c3)cc2)cc1] | 9.2 g (76%) | 778.30 |
| | 130 | 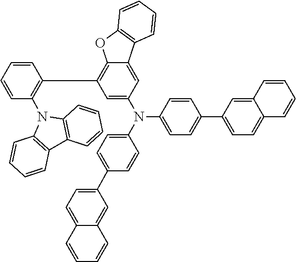 [SMILES: c1ccc(-n2c3ccccc3c3ccccc32)c(-c2cc(N(c3ccc(-c4ccc5ccccc5c4)cc3)c3ccc(-c4ccc5ccccc5c4)cc3)cc3c2oc2ccccc23)c1] | 10.2 g (79%) | 828.31 |
| | 131 | 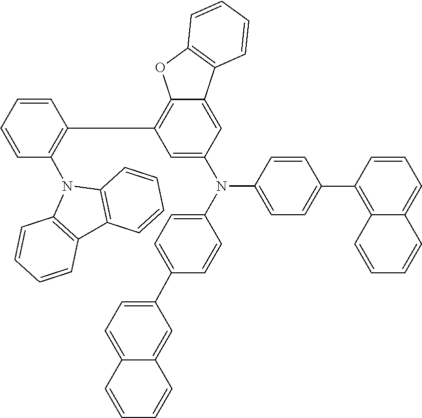 [SMILES: c1ccc(-n2c3ccccc3c3ccccc32)c(-c2cc(N(c3ccc(-c4ccc5ccccc5c4)cc3)c3ccc(-c4cccc5ccccc45)cc3)cc3c2oc2ccccc23)c1] | 9.8 g (76%) | 828.31 |
| | 132 | 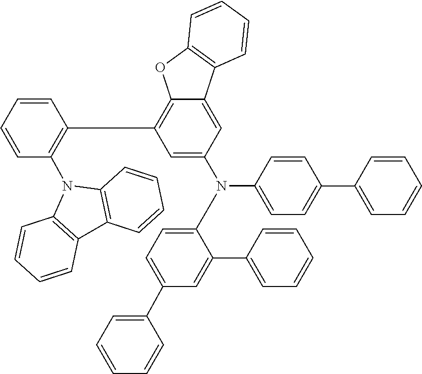 [SMILES: c1ccc(-c2ccc(N(c3cc(-c4ccccc4-n4c5ccccc5c5ccccc54)c4oc5ccccc5c4c3)c3ccc(-c4ccccc4)cc3-c3ccccc3)cc2)cc1] | 10.1 g (81%) | 804.31 |
| | 133 | 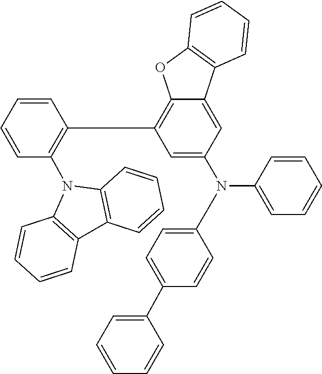 [SMILES: c1ccc(-c2ccc(N(c3ccccc3)c3cc(-c4ccccc4-n4c5ccccc5c5ccccc54)c4oc5ccccc5c4c3)cc2)cc1] | 7.8 g (77%) | 652.25 |
| | 134 | 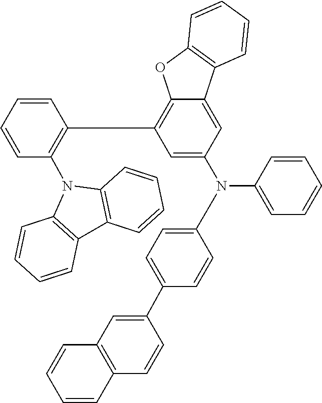 [SMILES: c1ccc(N(c2ccc(-c3ccc4ccccc4c3)cc2)c2cc(-c3ccccc3-n3c4ccccc4c4ccccc43)c3oc4ccccc4c3c2)cc1] | 8.1 g (74%) | 702.27 |
| | 135 | 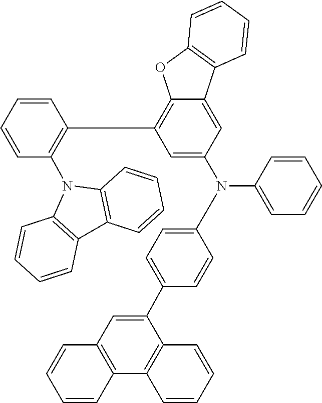 [SMILES: c1ccc(N(c2ccc(-c3cc4ccccc4c4ccccc34)cc2)c2cc(-c3ccccc3-n3c4ccccc4c4ccccc43)c3oc4ccccc4c3c2)cc1] | 9.3 g (79%) | 752.28 |
| | 136 | 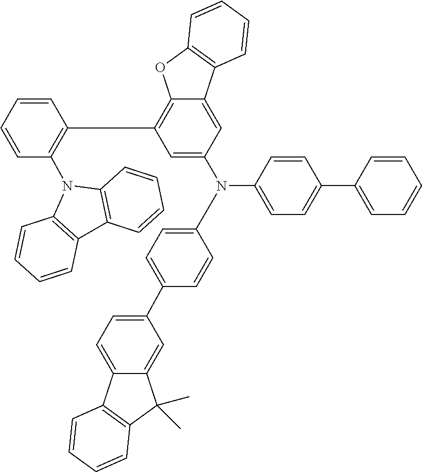 [SMILES: CC1(C)c2ccccc2-c2ccc(-c3ccc(N(c4ccc(-c5ccccc5)cc4)c4cc(-c5ccccc5-n5c6ccccc6c6ccccc65)c5oc6ccccc6c5c4)cc3)cc21] | 10.9 g (83%) | 844.35 |
| | 137 | 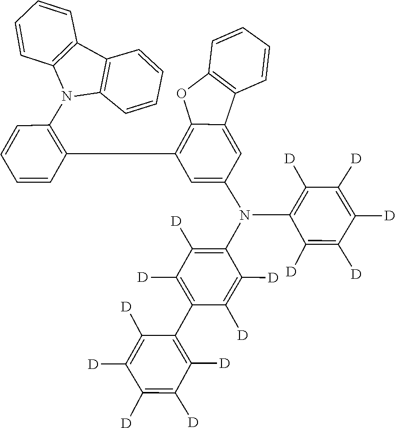 [SMILES: [2H]c1c([2H])c([2H])c(-c2c([2H])c([2H])c(N(c3cc(-c4ccccc4-n4c5ccccc5c5ccccc54)c4oc5ccccc5c4c3)c3c([2H])c([2H])c([2H])c([2H])c3[2H])c([2H])c2[2H])c([2H])c1[2H]] | 8.5 g (82%) | 666.34 |
| | 138 | 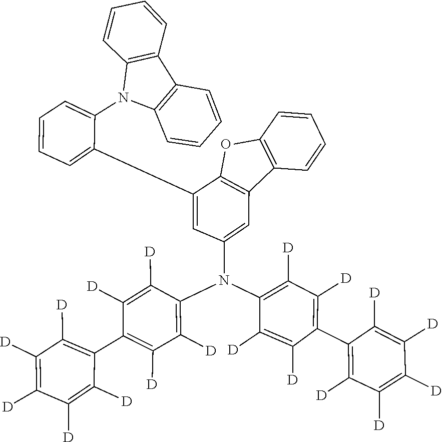 [SMILES: [2H]c1c([2H])c([2H])c(-c2c([2H])c([2H])c(N(c3cc(-c4ccccc4-n4c5ccccc5c5ccccc54)c4oc5ccccc5c4c3)c3c([2H])c([2H])c(-c4c([2H])c([2H])c([2H])c([2H])c4[2H])c([2H])c3[2H])c([2H])c2[2H])c([2H])c1[2H]] | 9.1 g (78%) | 746.40 |
| | 139 | 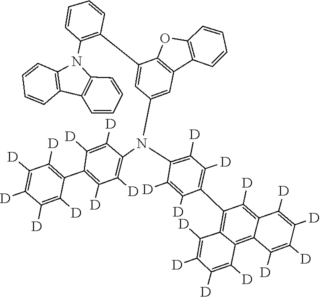 [SMILES: [2H]c1c([2H])c([2H])c(-c2c([2H])c([2H])c(N(c3cc(-c4ccccc4-n4c5ccccc5c5ccccc54)c4oc5ccccc5c4c3)c3c([2H])c([2H])c(-c4c([2H])c5c([2H])c([2H])c([2H])c([2H])c5c5c([2H])c([2H])c([2H])c([2H])c45)c([2H])c3[2H])c([2H])c2[2H])c([2H])c1[2H]] | 10.5 g (79%) | 850.45 |
| | 140 | 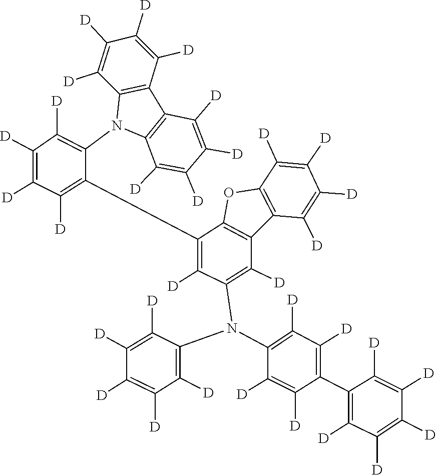 [SMILES: [2H]c1c([2H])c([2H])c(-c2c([2H])c([2H])c(N(c3c([2H])c([2H])c([2H])c([2H])c3[2H])c3c([2H])c(-c4c([2H])c([2H])c([2H])c([2H])c4-n4c5c([2H])c([2H])c([2H])c([2H])c5c5c([2H])c([2H])c([2H])c([2H])c54)c4oc5c([2H])c([2H])c([2H])c([2H])c5c4c3[2H])c([2H])c2[2H])c([2H])c1[2H]] | 7.7 g (72%) | 684.45 |
| | 141 | 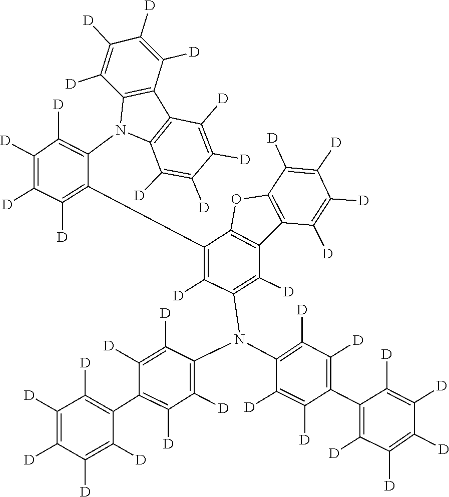 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c(-c4c([2H])c([2H])c([2H])c([2H])c4-n4c5c([2H])c([2H])c([2H])c([2H])c5c5c([2H])c([2H])c([2H])c([2H])c54)c4oc5c([2H])c([2H])c([2H])c([2H])c5c4c3[2H])c([2H])c2[2H])c([2H])c1[2H]] | 8.9 g (75%) | 764.51 |
| | 142 | 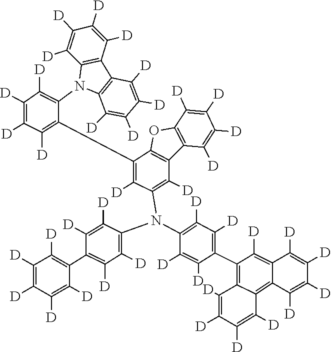 [SMILES: [2H]c1c([2H])c([2H])c(-c2c([2H])c([2H])c(N(c3c([2H])c([2H])c(-c4c([2H])c5c([2H])c([2H])c([2H])c([2H])c5c5c([2H])c([2H])c([2H])c([2H])c45)c([2H])c3[2H])c3c([2H])c(-c4c([2H])c([2H])c([2H])c([2H])c4-n4c5c([2H])c([2H])c([2H])c([2H])c5c5c([2H])c([2H])c([2H])c([2H])c54)c4oc5c([2H])c([2H])c([2H])c([2H])c5c4c3[2H])c([2H])c2[2H])c([2H])c1[2H]] | 10.4 g (77%) | 868.57 |

TABLE 13

| | SUB 2-11 | |
|---|---|---|
| Compound | SUB 2-23 | SUB 1 |
| 143 | 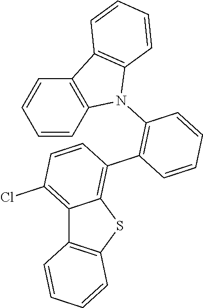 [SMILES: Clc1ccc(-c2ccccc2-n2c3ccccc3c3ccccc32)c2sc3ccccc3c12] | 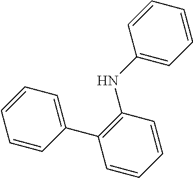 [SMILES: c1ccc(Nc2ccccc2-c2ccccc2)cc1] |
| 144 | | 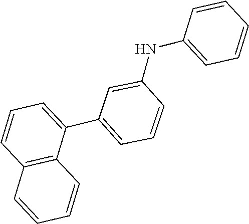 [SMILES: c1ccc(Nc2cccc(-c3cccc4ccccc34)c2)cc1] |
| 145 | | 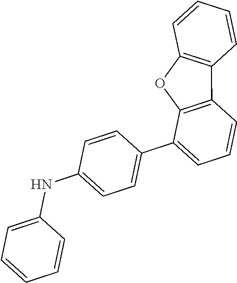 [SMILES: c1ccc(Nc2ccc(-c3cccc4c3oc3ccccc34)cc2)cc1] |
| 146 | | 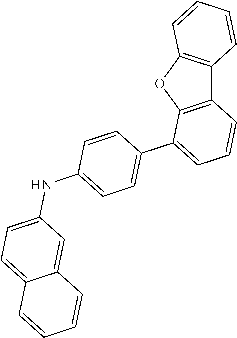 [SMILES: c1ccc2cc(Nc3ccc(-c4cccc5c4oc4ccccc45)cc3)ccc2c1] |
| 147 | | 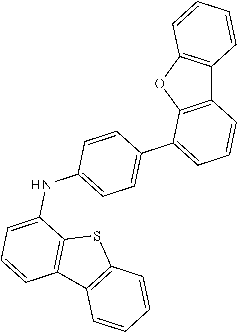 [SMILES: c1ccc2c(c1)oc1c(-c3ccc(Nc4cccc5c4sc4ccccc45)cc3)cccc12] |
| 148 | | 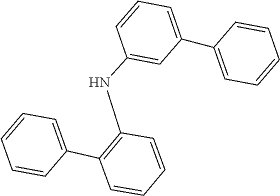 [SMILES: c1ccc(-c2cccc(Nc3ccccc3-c3ccccc3)c2)cc1] |
| 149 | | 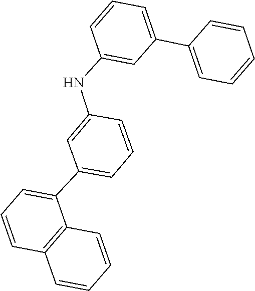 [SMILES: c1ccc(-c2cccc(Nc3cccc(-c4cccc5ccccc45)c3)c2)cc1] |
| 150 | | 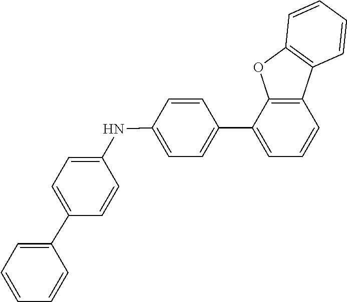 [SMILES: c1ccc(-c2ccc(Nc3ccc(-c4cccc5c4oc4ccccc45)cc3)cc2)cc1] |
| 151 | | 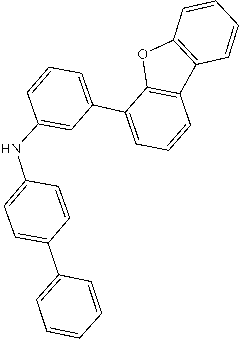 [SMILES: c1ccc(-c2ccc(Nc3cccc(-c4cccc5c4oc4ccccc45)c3)cc2)cc1] |
| 152 | | 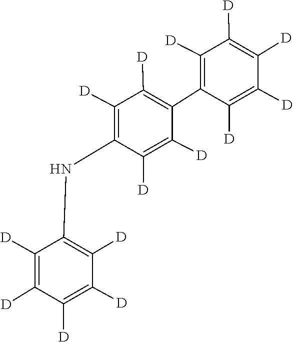 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] |
| 153 | | 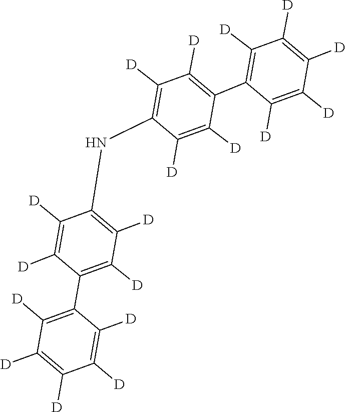 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] |
| 154 | 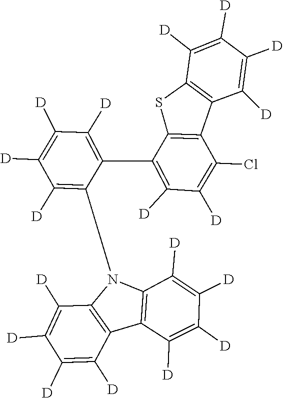 [SMILES: [2H]c1c([2H])c([2H])c(-n2c3c([2H])c([2H])c([2H])c([2H])c3c3c([2H])c([2H])c([2H])c([2H])c32)c(-c2c([2H])c([2H])c(Cl)c3c2sc2c([2H])c([2H])c([2H])c([2H])c23)c1[2H]] | 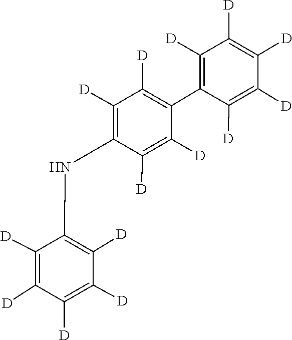 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] |
| 155 | | 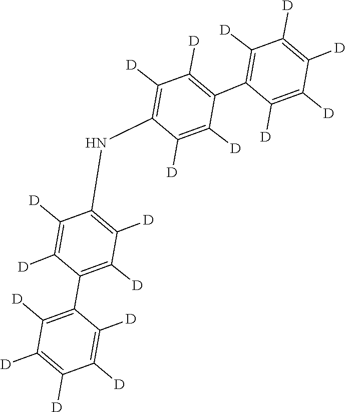 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] |

| | | Obtained | |
|---|---|---|---|
| Compound | Product | amount | [M + H]⁺ |
| 143 | 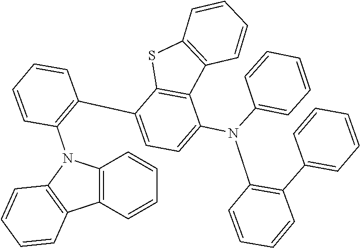 [SMILES: c1ccc(-c2ccccc2N(c2ccccc2)c2ccc(-c3ccccc3-n3c4ccccc4c4ccccc43)c3sc4ccccc4c23)cc1] | 8.5 g (82%) | 668.23 |
| 144 | 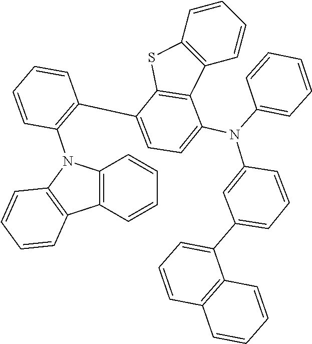 [SMILES: c1ccc(N(c2cccc(-c3cccc4ccccc34)c2)c2ccc(-c3ccccc3-n3c4ccccc4c4ccccc43)c3sc4ccccc4c23)cc1] | 8.9 g (80%) | 718.24 |
| 145 | 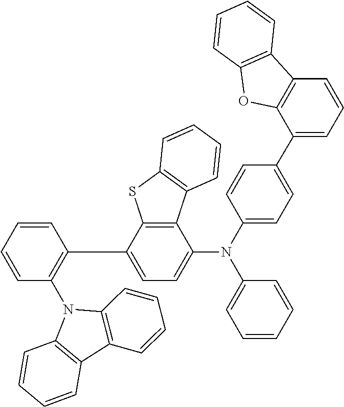 [SMILES: c1ccc(N(c2ccc(-c3cccc4c3oc3ccccc34)cc2)c2ccc(-c3ccccc3-n3c4ccccc4c4ccccc43)c3sc4ccccc4c23)cc1] | 9.0 g (76%) | 758.24 |
| 146 | 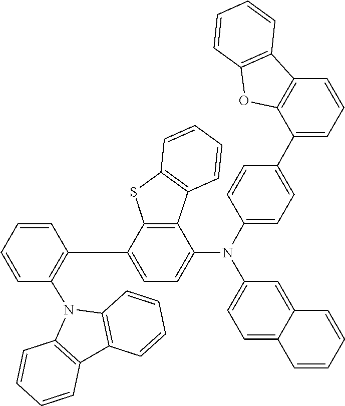 [SMILES: c1ccc(-n2c3ccccc3c3ccccc32)c(-c2ccc(N(c3ccc(-c4cccc5c4oc4ccccc45)cc3)c3ccc4ccccc4c3)c3c2sc2ccccc23)c1] | 9.3 g (74%) | 808.25 |
| 147 | 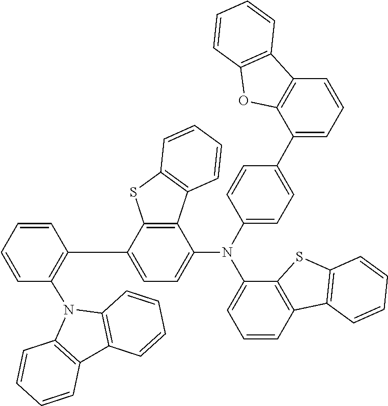 [SMILES: c1ccc(-n2c3ccccc3c3ccccc32)c(-c2ccc(N(c3ccc(-c4cccc5c4oc4ccccc45)cc3)c3cccc4c3sc3ccccc34)c3c2sc2ccccc23)c1] | 10.2 g (74%) | 864.23 |
| 148 | 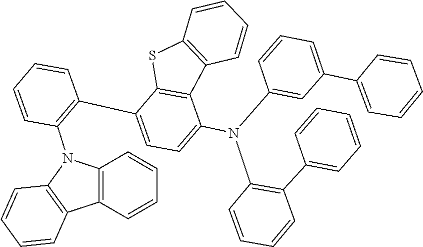 [SMILES: c1ccc(-c2cccc(N(c3ccccc3-c3ccccc3)c3ccc(-c4ccccc4-n4c5ccccc5c5ccccc54)c4sc5ccccc5c34)c2)cc1] | 9.4 g (81%) | 744.26 |
| 149 | 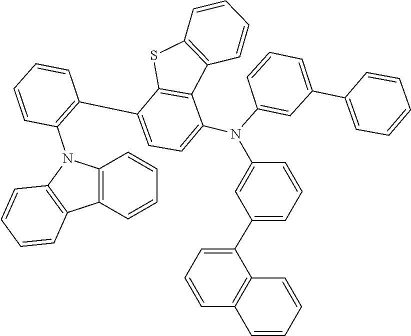 [SMILES: c1ccc(-c2cccc(N(c3cccc(-c4cccc5ccccc45)c3)c3ccc(-c4ccccc4-n4c5ccccc5c5ccccc54)c4sc5ccccc5c34)c2)cc1] | 9.8 g (79%) | 794.28 |
| 150 | 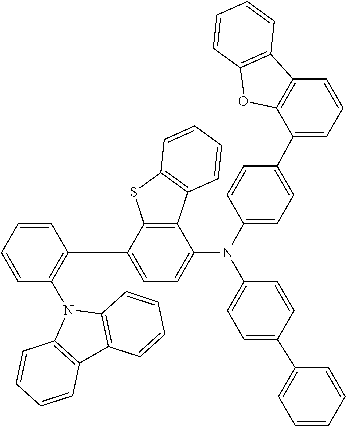 [SMILES: c1ccc(-c2ccc(N(c3ccc(-c4cccc5c4oc4ccccc45)cc3)c3ccc(-c4ccccc4-n4c5ccccc5c5ccccc54)c4sc5ccccc5c34)cc2)cc1] | 9.9 g (76%) | 834.27 |
| 151 | 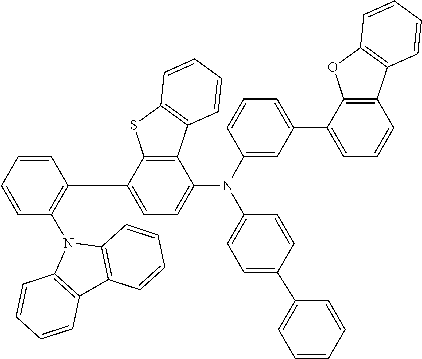 [SMILES: c1ccc(-c2ccc(N(c3cccc(-c4cccc5c4oc4ccccc45)c3)c3ccc(-c4ccccc4-n4c5ccccc5c5ccccc54)c4sc5ccccc5c34)cc2)cc1] | 10.0 g (77%) | 834.27 |
| 152 | 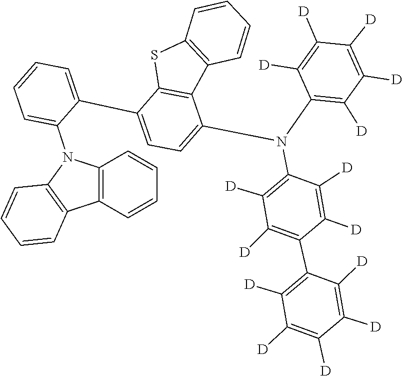 [SMILES: [2H]c1c([2H])c([2H])c(-c2c([2H])c([2H])c(N(c3c([2H])c([2H])c([2H])c([2H])c3[2H])c3ccc(-c4ccccc4-n4c5ccccc5c5ccccc54)c4sc5ccccc5c34)c([2H])c2[2H])c([2H])c1[2H]] | 8.5 g (80%) | 682.32 |
| 153 | 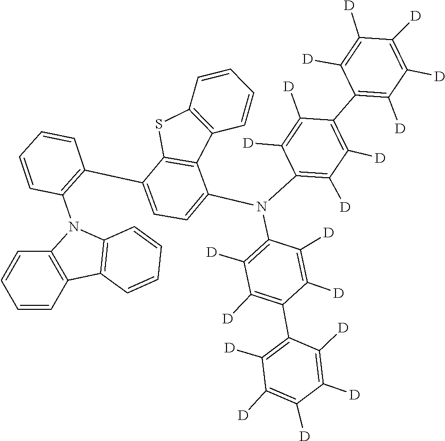 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3ccc(-c4ccccc4-n4c5ccccc5c5ccccc54)c4sc5ccccc5c34)c([2H])c2[2H])c([2H])c1[2H]] | 9.0 g (76%) | 762.37 |
| 154 | 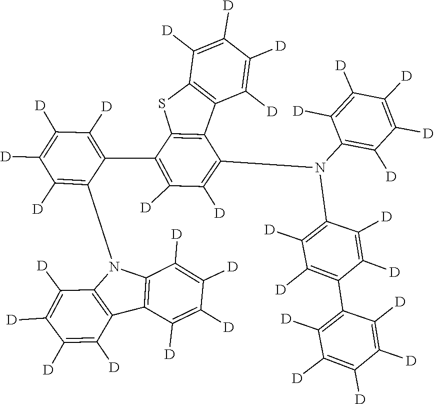 [SMILES: [2H]c1c([2H])c([2H])c(-c2c([2H])c([2H])c(N(c3c([2H])c([2H])c([2H])c([2H])c3[2H])c3c([2H])c([2H])c(-c4c([2H])c([2H])c([2H])c([2H])c4-n4c5c([2H])c([2H])c([2H])c([2H])c5c5c([2H])c([2H])c([2H])c([2H])c54)c4sc5c([2H])c([2H])c([2H])c([2H])c5c34)c([2H])c2[2H])c([2H])c1[2H]] | 9.0 g (83%) | 700.43 |
| 155 | 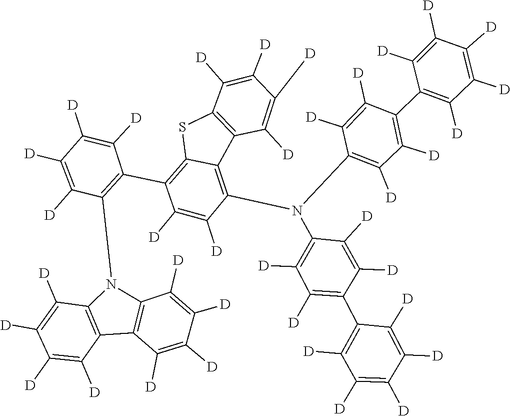 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c(-c4c([2H])c([2H])c([2H])c([2H])c4-n4c5c([2H])c([2H])c([2H])c([2H])c5c5c([2H])c([2H])c([2H])c([2H])c54)c4sc5c([2H])c([2H])c([2H])c([2H])c5c34)c([2H])c2[2H])c([2H])c1[2H]] | 9.8 g (81%) | 780.49 |

TABLE 14

| | SUB 2-12 | |
|---|---|---|
| Compound | SUB 2-24 | SUB 1 |
| 156 | 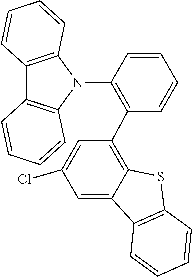 [SMILES: Clc1cc(-c2ccccc2-n2c3ccccc3c3ccccc32)c2sc3ccccc3c2c1] | 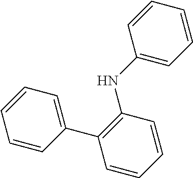 [SMILES: c1ccc(Nc2ccccc2-c2ccccc2)cc1] |
| 157 | | 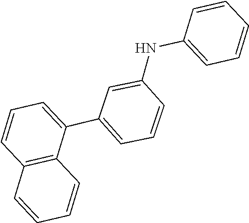 [SMILES: c1ccc(Nc2cccc(-c3cccc4ccccc34)c2)cc1] |
| 158 | | 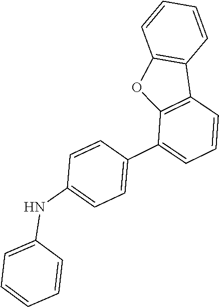 [SMILES: c1ccc(Nc2ccc(-c3cccc4c3oc3ccccc34)cc2)cc1] |
| 159 | | 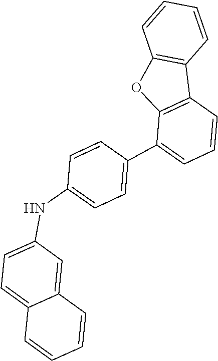 [SMILES: c1ccc2cc(Nc3ccc(-c4cccc5c4oc4ccccc45)cc3)ccc2c1] |
| 160 | | 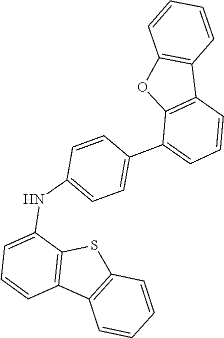 [SMILES: c1ccc2c(c1)oc1c(-c3ccc(Nc4cccc5c4sc4ccccc45)cc3)cccc12] |
| 161 | | 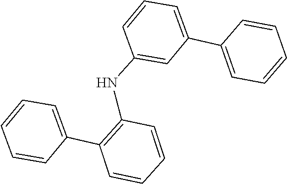 [SMILES: c1ccc(-c2cccc(Nc3ccccc3-c3ccccc3)c2)cc1] |
| 162 | | 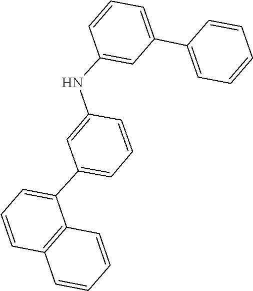 [SMILES: c1ccc(-c2cccc(Nc3cccc(-c4cccc5ccccc45)c3)c2)cc1] |
| 163 | | 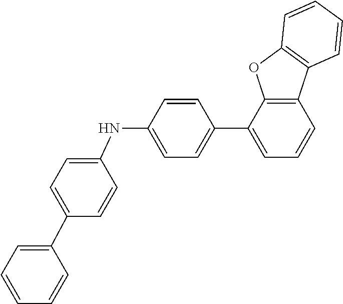 [SMILES: c1ccc(-c2ccc(Nc3ccc(-c4cccc5c4oc4ccccc45)cc3)cc2)cc1] |
| 164 | | 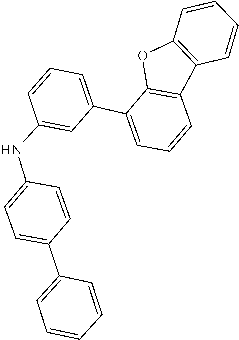 [SMILES: c1ccc(-c2ccc(Nc3cccc(-c4cccc5c4oc4ccccc45)c3)cc2)cc1] |
| 165 | | 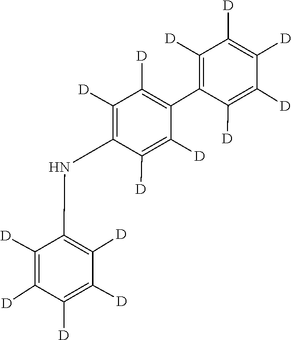 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] |
| 166 | | 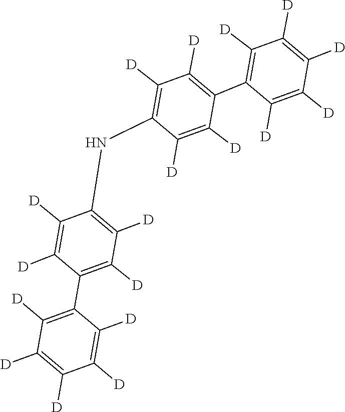 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] |
| 167 | 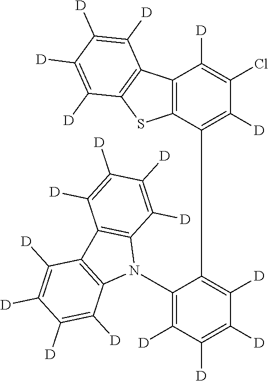 | 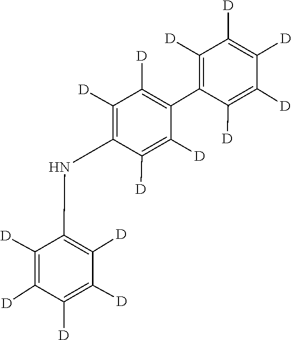 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] |
| 168 | | 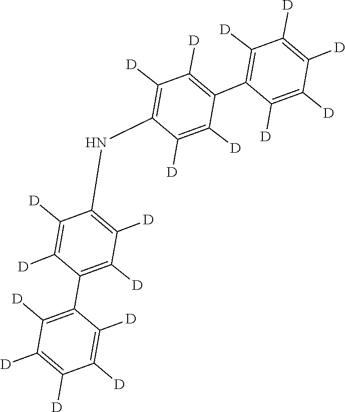 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] |

| | | Obtained | |
|---|---|---|---|
| Compound | Product | amount | [M + H]⁺ |
| 156 | 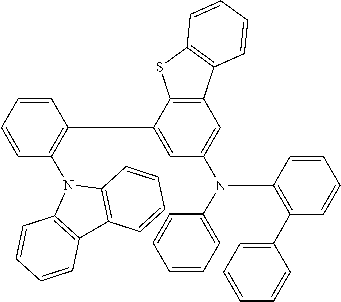 [SMILES: c1ccc(-c2ccccc2N(c2ccccc2)c2cc(-c3ccccc3-n3c4ccccc4c4ccccc43)c3sc4ccccc4c3c2)cc1] | 7.9 g (76%) | 668.23 |
| 157 | 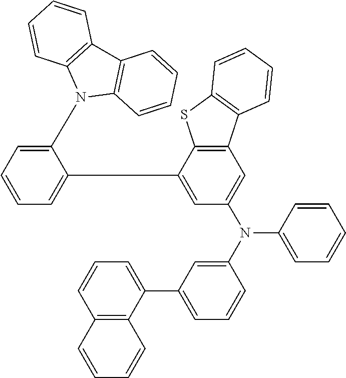 [SMILES: c1ccc(N(c2cccc(-c3cccc4ccccc34)c2)c2cc(-c3ccccc3-n3c4ccccc4c4ccccc43)c3sc4ccccc4c3c2)cc1] | 8.5 g (76%) | 718.24 |
| 158 | 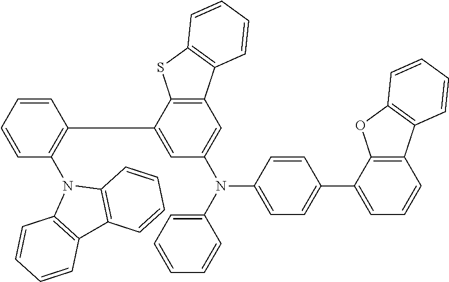 [SMILES: c1ccc(N(c2ccc(-c3cccc4c3oc3ccccc34)cc2)c2cc(-c3ccccc3-n3c4ccccc4c4ccccc43)c3sc4ccccc4c3c2)cc1] | 8.7 g (74%) | 758.24 |
| 159 | 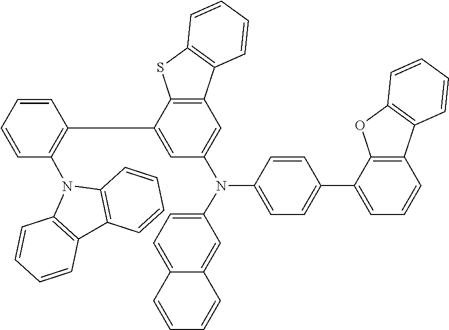 [SMILES: c1ccc(-n2c3ccccc3c3ccccc32)c(-c2cc(N(c3ccc(-c4cccc5c4oc4ccccc45)cc3)c3ccc4ccccc4c3)cc3c2sc2ccccc23)c1] | 8.6 g (68%) | 808.25 |
| 160 | 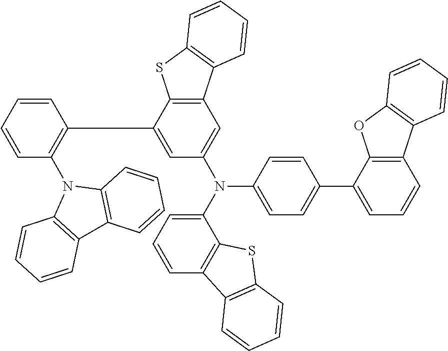 [SMILES: c1ccc(-n2c3ccccc3c3ccccc32)c(-c2cc(N(c3ccc(-c4cccc5c4oc4ccccc45)cc3)c3cccc4c3sc3ccccc34)cc3c2sc2ccccc23)c1] | 9.8 g (73%) | 864.23 |
| 161 | 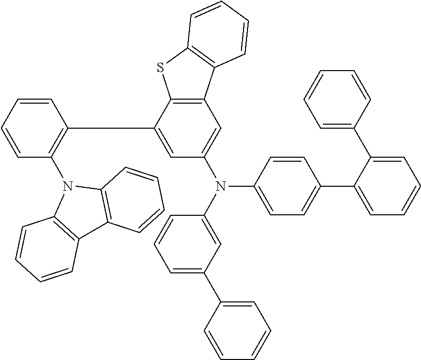 [SMILES: c1ccc(-c2cccc(N(c3ccc(-c4ccccc4-c4ccccc4)cc3)c3cc(-c4ccccc4-n4c5ccccc5c5ccccc54)c4sc5ccccc5c4c3)c2)cc1] | 8.2 g (71%) | 744.26 |
| 162 | 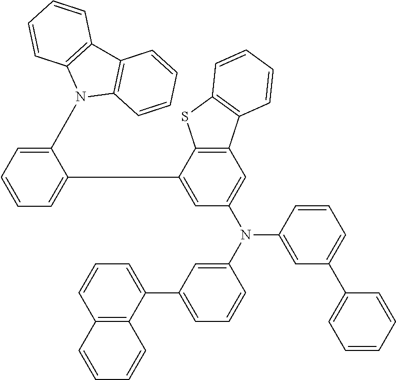 [SMILES: c1ccc(-c2cccc(N(c3cccc(-c4cccc5ccccc45)c3)c3cc(-c4ccccc4-n4c5ccccc5c5ccccc54)c4sc5ccccc5c4c3)c2)cc1] | 9.1 g (74%) | 794.28 |
| 163 | 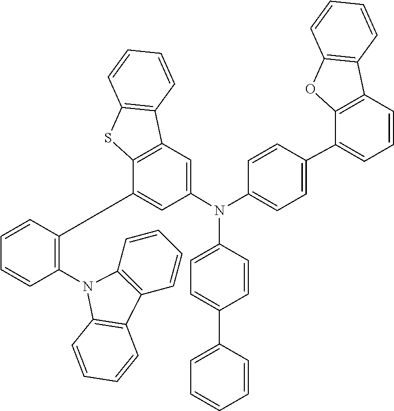 [SMILES: c1ccc(-c2ccc(N(c3ccc(-c4cccc5c4oc4ccccc45)cc3)c3cc(-c4ccccc4-n4c5ccccc5c5ccccc54)c4sc5ccccc5c4c3)cc2)cc1] | 10.4 g (80%) | 834.27 |
| 164 | 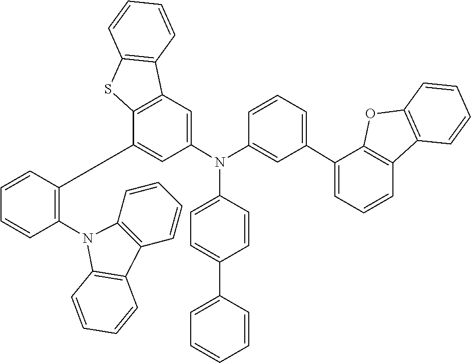 [SMILES: c1ccc(-c2ccc(N(c3cccc(-c4cccc5c4oc4ccccc45)c3)c3cc(-c4ccccc4-n4c5ccccc5c5ccccc54)c4sc5ccccc5c4c3)cc2)cc1] | 9.3 g (72%) | 834.27 |
| 165 | 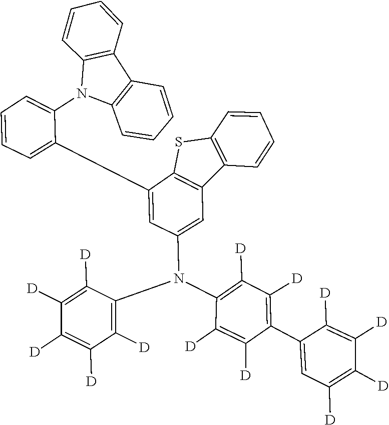 [SMILES: [2H]c1cc(-c2c([2H])c([2H])c(N(c3cc(-c4ccccc4-n4c5ccccc5c5ccccc54)c4sc5ccccc5c4c3)c3c([2H])c([2H])c([2H])c([2H])c3[2H])c([2H])c2[2H])c([2H])c([2H])c1[2H]] | 8.1 g (76%) | 681.31 |
| 166 | 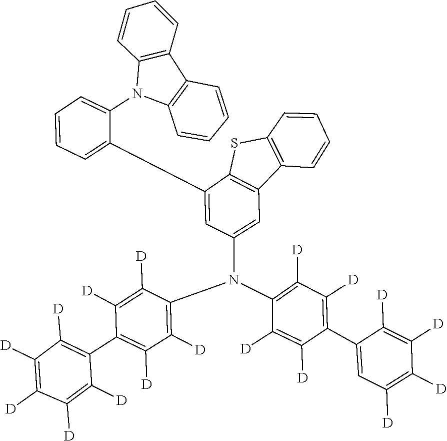 [SMILES: [2H]c1cc(-c2c([2H])c([2H])c(N(c3cc(-c4ccccc4-n4c5ccccc5c5ccccc54)c4sc5ccccc5c4c3)c3c([2H])c([2H])c(-c4c([2H])c([2H])c([2H])c([2H])c4[2H])c([2H])c3[2H])c([2H])c2[2H])c([2H])c([2H])c1[2H]] | 8.5 g (72%) | 761.37 |
| 167 | 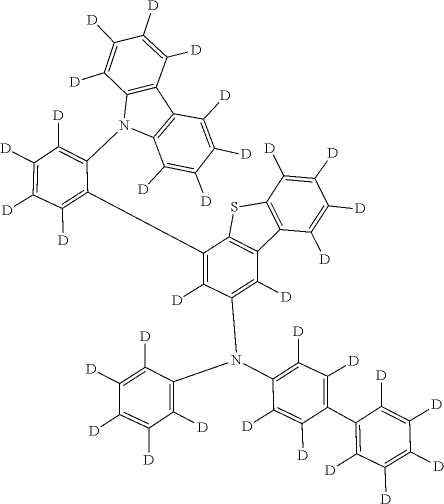 [SMILES: [2H]c1c([2H])c([2H])c(-c2c([2H])c([2H])c(N(c3c([2H])c([2H])c([2H])c([2H])c3[2H])c3c([2H])c(-c4c([2H])c([2H])c([2H])c([2H])c4-n4c5c([2H])c([2H])c([2H])c([2H])c5c5c([2H])c([2H])c([2H])c([2H])c54)c4sc5c([2H])c([2H])c([2H])c([2H])c5c4c3[2H])c([2H])c2[2H])c([2H])c1[2H]] | 8.6 g (79%) | 700.43 |
| 168 | 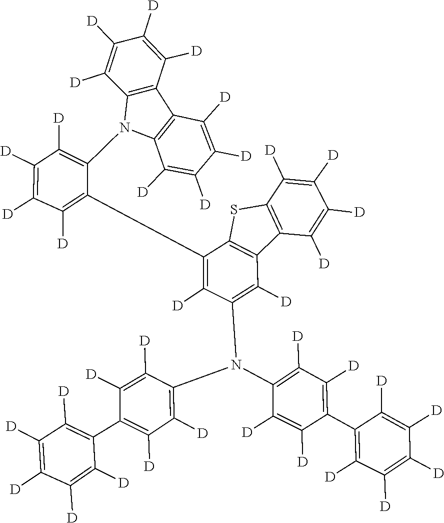 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c(-c4c([2H])c([2H])c([2H])c([2H])c4-n4c5c([2H])c([2H])c([2H])c([2H])c5c5c([2H])c([2H])c([2H])c([2H])c54)c4sc5c([2H])c([2H])c([2H])c([2H])c5c4c3[2H])c([2H])c2[2H])c([2H])c1[2H]] | 9.7 g (80%) | 780.49 |

TABLE 15

| | SUB 2-25 | |
|---|---|---|
| Compound | SUB 2-31 | SUB 1 |
| 169 | 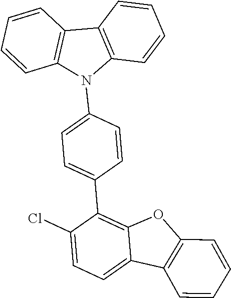 [SMILES: Clc1ccc2c(oc3ccccc32)c1-c1ccc(-n2c3ccccc3c3ccccc32)cc1] | 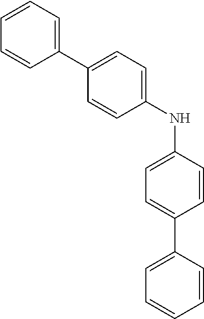 [SMILES: c1ccc(-c2ccc(Nc3ccc(-c4ccccc4)cc3)cc2)cc1] |
| 170 | | 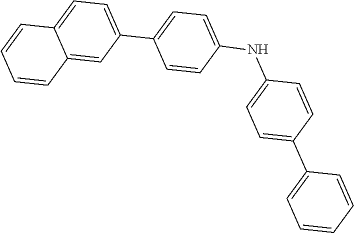 [SMILES: c1ccc(-c2ccc(Nc3ccc(-c4ccc5ccccc5c4)cc3)cc2)cc1] |
| 171 | | 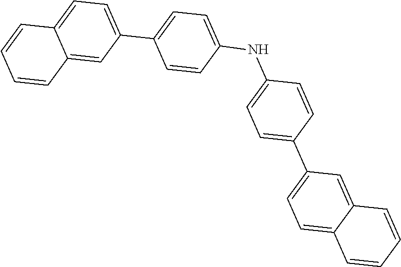 [SMILES: c1ccc2cc(-c3ccc(Nc4ccc(-c5ccc6ccccc6c5)cc4)cc3)ccc2c1] |
| 172 | | 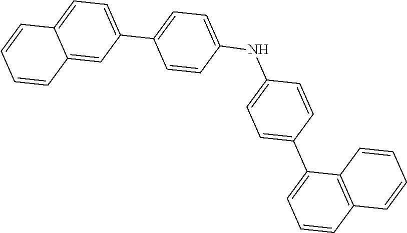 [SMILES: c1ccc2cc(-c3ccc(Nc4ccc(-c5cccc6ccccc56)cc4)cc3)ccc2c1] |
| 173 | | 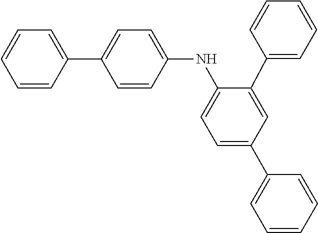 [SMILES: c1ccc(-c2ccc(Nc3ccc(-c4ccccc4)cc3-c3ccccc3)cc2)cc1] |
| 174 | | 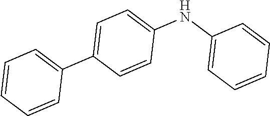 [SMILES: c1ccc(Nc2ccc(-c3ccccc3)cc2)cc1] |
| 175 | | 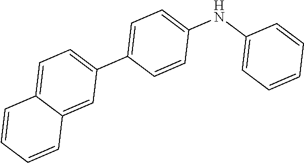 [SMILES: c1ccc(Nc2ccc(-c3ccc4ccccc4c3)cc2)cc1] |
| 176 | | 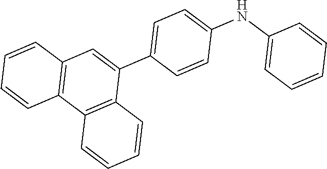 [SMILES: c1ccc(Nc2ccc(-c3cc4ccccc4c4ccccc34)cc2)cc1] |
| 177 | | 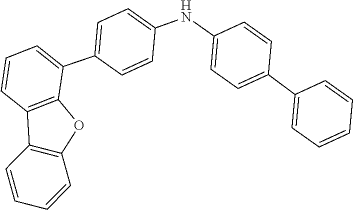 [SMILES: c1ccc(-c2ccc(Nc3ccc(-c4cccc5c4oc4ccccc45)cc3)cc2)cc1] |
| 178 | | 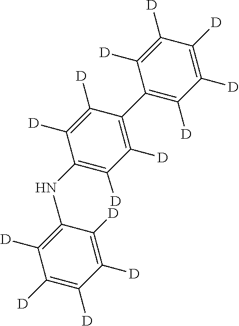 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] |
| 179 | | 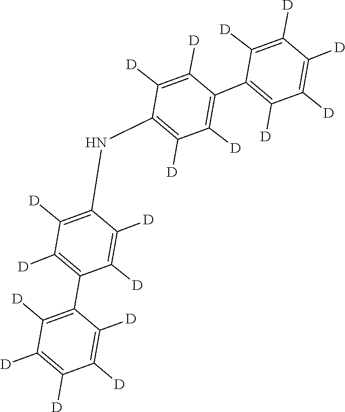 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] |
| 180 | | 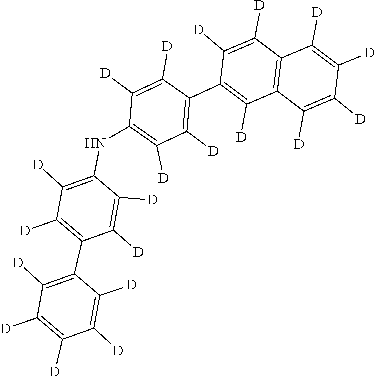 [SMILES: [2H]c1c([2H])c([2H])c(-c2c([2H])c([2H])c(Nc3c([2H])c([2H])c(-c4c([2H])c([2H])c5c([2H])c([2H])c([2H])c([2H])c5c4[2H])c([2H])c3[2H])c([2H])c2[2H])c([2H])c1[2H]] |
| 181 | 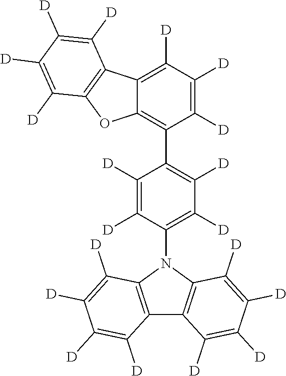 [SMILES: [2H]c1c([2H])c(-n2c3c([2H])c([2H])c([2H])c([2H])c3c3c([2H])c([2H])c([2H])c([2H])c32)c([2H])c([2H])c1-c1c([2H])c([2H])c([2H])c2c1oc1c([2H])c([2H])c([2H])c([2H])c12] | 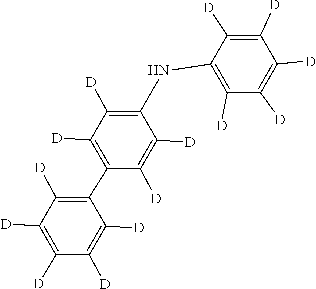 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] |
| 182 | | 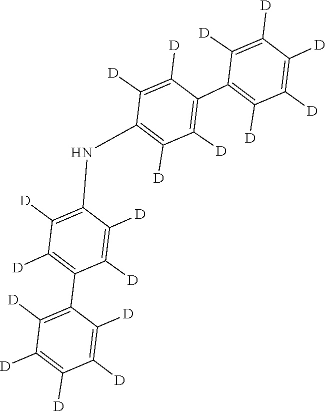 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] |
| 183 | | 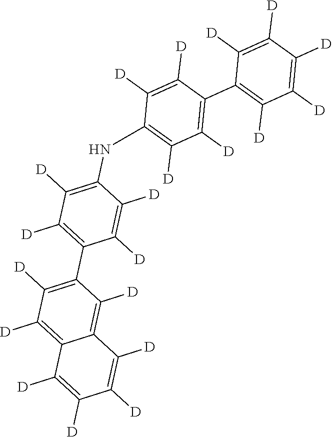 [SMILES: [2H]c1c([2H])c([2H])c(-c2c([2H])c([2H])c(Nc3c([2H])c([2H])c(-c4c([2H])c([2H])c5c([2H])c([2H])c([2H])c([2H])c5c4[2H])c([2H])c3[2H])c([2H])c2[2H])c([2H])c1[2H]] |

| | | Obtained | |
|---|---|---|---|
| Compound | Product | amount | [M + H]⁺ |
| 169 | 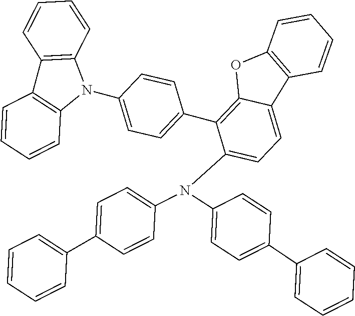 [SMILES: c1ccc(-c2ccc(N(c3ccc(-c4ccccc4)cc3)c3ccc4c(oc5ccccc54)c3-c3ccc(-n4c5ccccc5c5ccccc54)cc3)cc2)cc1] | 7.9 g (70%) | 728.28 |
| 170 | 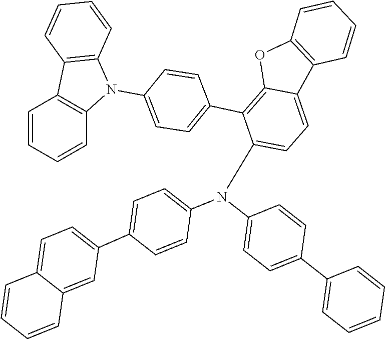 [SMILES: c1ccc(-c2ccc(N(c3ccc(-c4ccc5ccccc5c4)cc3)c3ccc4c(oc5ccccc54)c3-c3ccc(-n4c5ccccc5c5ccccc54)cc3)cc2)cc1] | 8.1 g (67%) | 778.30 |
| 171 | 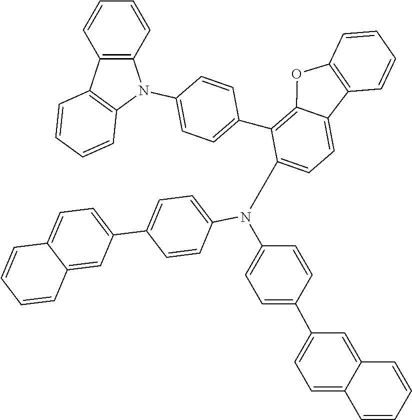 [SMILES: c1ccc2cc(-c3ccc(N(c4ccc(-c5ccc6ccccc6c5)cc4)c4ccc5c(oc6ccccc65)c4-c4ccc(-n5c6ccccc6c6ccccc65)cc4)cc3)ccc2c1] | 8.0 g (62%) | 828.31 |
| 172 | 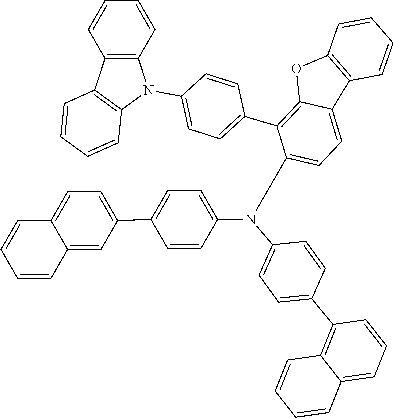 [SMILES: c1ccc2cc(-c3ccc(N(c4ccc(-c5cccc6ccccc56)cc4)c4ccc5c(oc6ccccc65)c4-c4ccc(-n5c6ccccc6c6ccccc65)cc4)cc3)ccc2c1] | 8.2 g (64%) | 828.31 |
| 173 | 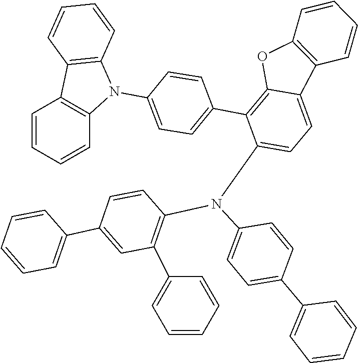 [SMILES: c1ccc(-c2ccc(N(c3ccc(-c4ccccc4)cc3-c3ccccc3)c3ccc4c(oc5ccccc54)c3-c3ccc(-n4c5ccccc5c5ccccc54)cc3)cc2)cc1] | 8.4 g (67%) | 804.31 |
| 174 | 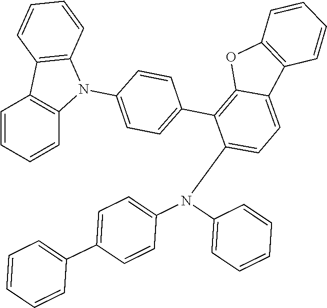 [SMILES: c1ccc(-c2ccc(N(c3ccccc3)c3ccc4c(oc5ccccc54)c3-c3ccc(-n4c5ccccc5c5ccccc54)cc3)cc2)cc1] | 7.0 g (72%) | 625.25 |
| 175 | 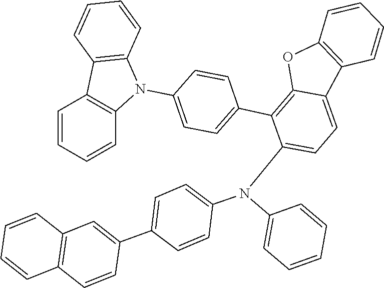 [SMILES: c1ccc(N(c2ccc(-c3ccc4ccccc4c3)cc2)c2ccc3c(oc4ccccc43)c2-c2ccc(-n3c4ccccc4c4ccccc43)cc2)cc1] | 7.3 g (67%) | 702.27 |
| 176 | 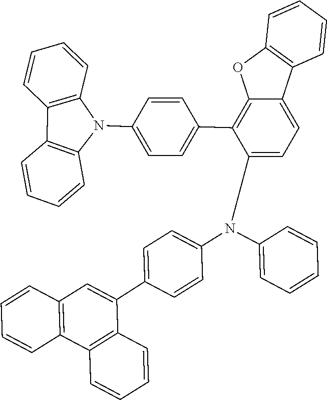 [SMILES: c1ccc(N(c2ccc(-c3cc4ccccc4c4ccccc34)cc2)c2ccc3c(oc4ccccc43)c2-c2ccc(-n3c4ccccc4c4ccccc43)cc2)cc1] | 7.7 g (66%) | 752.28 |
| 177 | 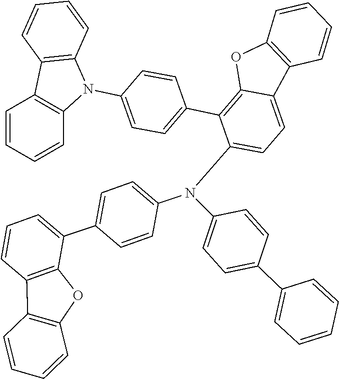 [SMILES: c1ccc(-c2ccc(N(c3ccc(-c4cccc5c4oc4ccccc45)cc3)c3ccc4c(oc5ccccc54)c3-c3ccc(-n4c5ccccc5c5ccccc54)cc3)cc2)cc1] | 8.8 g (69%) | 818.29 |
| 178 | 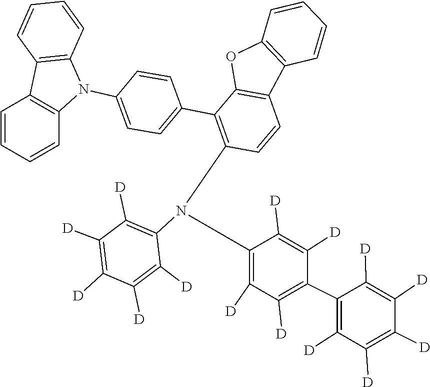 [SMILES: [2H]c1c([2H])c([2H])c(-c2c([2H])c([2H])c(N(c3ccc4c(oc5ccccc54)c3-c3ccc(-n4c5ccccc5c5ccccc54)cc3)c3c([2H])c([2H])c([2H])c([2H])c3[2H])c([2H])c2[2H])c([2H])c1[2H]] | 7.7 g (74%) | 666.34 |
| 179 | 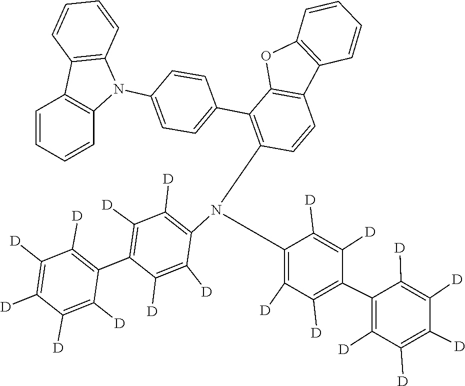 [SMILES: [2H]c1c([2H])c([2H])c(-c2c([2H])c([2H])c(N(c3ccc4c(oc5ccccc54)c3-c3ccc(-n4c5ccccc5c5ccccc54)cc3)c3c([2H])c([2H])c(-c4c([2H])c([2H])c([2H])c([2H])c4[2H])c([2H])c3[2H])c([2H])c2[2H])c([2H])c1[2H]] | 8.4 g (72%) | 746.40 |
| 180 | 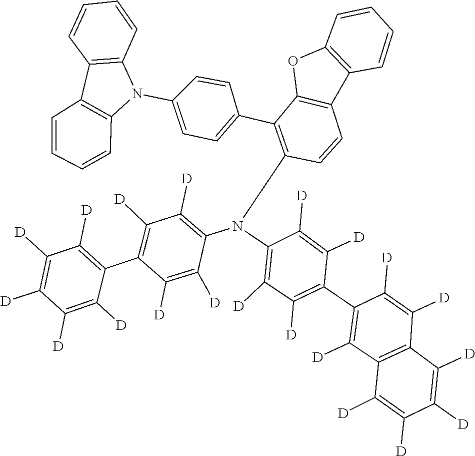 [SMILES: [2H]c1c([2H])c([2H])c(-c2c([2H])c([2H])c(N(c3ccc4c(oc5ccccc54)c3-c3ccc(-n4c5ccccc5c5ccccc54)cc3)c3c([2H])c([2H])c(-c4c([2H])c([2H])c5c([2H])c([2H])c([2H])c([2H])c5c4[2H])c([2H])c3[2H])c([2H])c2[2H])c([2H])c1[2H]] | 8.3 g (67%) | 798.42 |
| 181 | 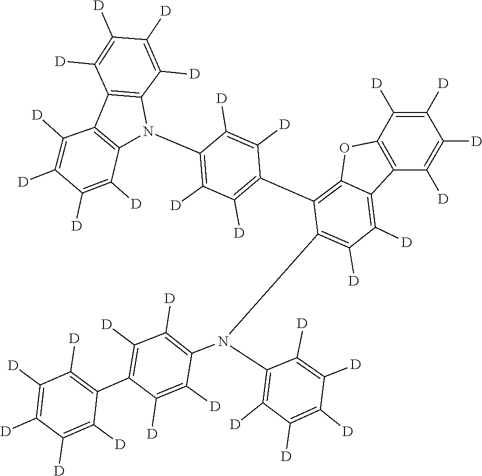 [SMILES: [2H]c1c([2H])c([2H])c(-c2c([2H])c([2H])c(N(c3c([2H])c([2H])c([2H])c([2H])c3[2H])c3c([2H])c([2H])c4c(oc5c([2H])c([2H])c([2H])c([2H])c54)c3-c3c([2H])c([2H])c(-n4c5c([2H])c([2H])c([2H])c([2H])c5c5c([2H])c([2H])c([2H])c([2H])c54)c([2H])c3[2H])c([2H])c2[2H])c([2H])c1[2H]] | 7.5 g (70%) | 684.45 |
| 182 | 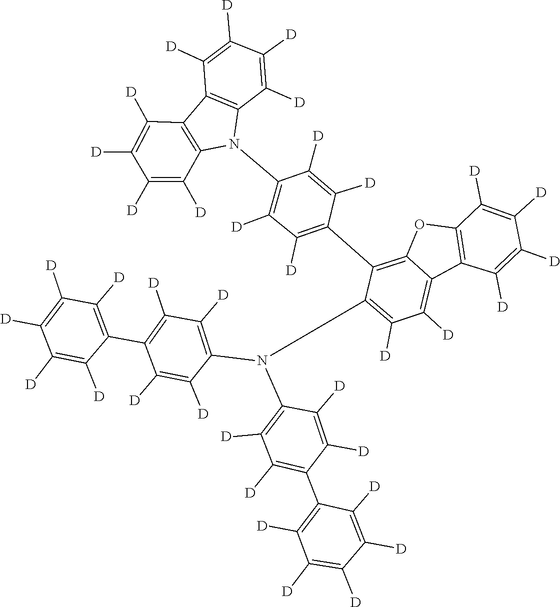 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4c(oc5c([2H])c([2H])c([2H])c([2H])c54)c3-c3c([2H])c([2H])c(-n4c5c([2H])c([2H])c([2H])c([2H])c5c5c([2H])c([2H])c([2H])c([2H])c54)c([2H])c3[2H])c([2H])c2[2H])c([2H])c1[2H]] | 9.0 g (76%) | 764.51 |
| 183 | 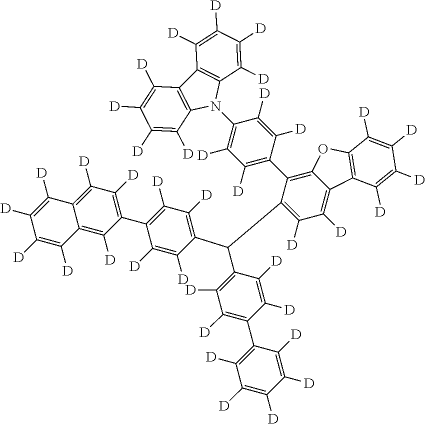 [SMILES: [2H]c1c([2H])c([2H])c(-c2c([2H])c([2H])c(C(c3c([2H])c([2H])c(-c4c([2H])c([2H])c5c([2H])c([2H])c([2H])c([2H])c5c4[2H])c([2H])c3[2H])c3c([2H])c([2H])c4c(oc5c([2H])c([2H])c([2H])c([2H])c54)c3-c3c([2H])c([2H])c(-n4c5c([2H])c([2H])c([2H])c([2H])c5c5c([2H])c([2H])c([2H])c([2H])c54)c([2H])c3[2H])c([2H])c2[2H])c([2H])c1[2H]] | 9.0 g (71%) | 816.54 |

TABLE 16

| | SUB 2-27 | |
|---|---|---|
| Compound | SUB 2-33 | SUB 1 |
| 184 | 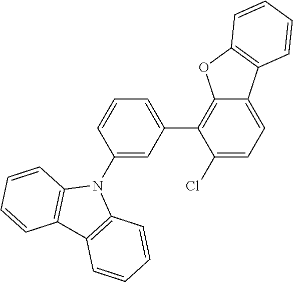 [SMILES: Clc1ccc2c(oc3ccccc32)c1-c1cccc(-n2c3ccccc3c3ccccc32)c1] | 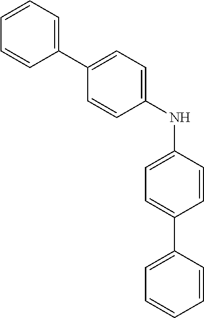 [SMILES: c1ccc(-c2ccc(Nc3ccc(-c4ccccc4)cc3)cc2)cc1] |
| 185 | | 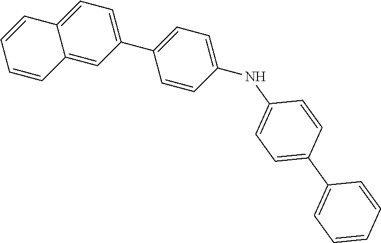 [SMILES: c1ccc(-c2ccc(Nc3ccc(-c4ccc5ccccc5c4)cc3)cc2)cc1] |
| 186 | | 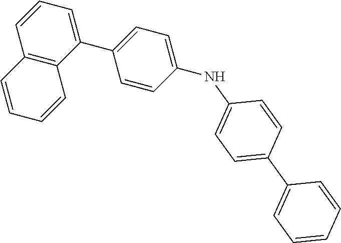 [SMILES: c1ccc(-c2ccc(Nc3ccc(-c4cccc5ccccc45)cc3)cc2)cc1] |
| 187 | | 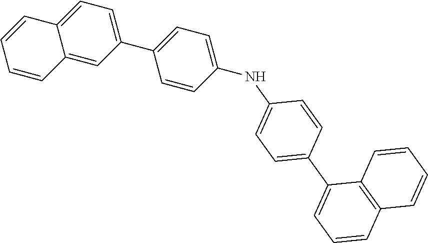 [SMILES: c1ccc2cc(-c3ccc(Nc4ccc(-c5cccc6ccccc56)cc4)cc3)ccc2c1] |
| 188 | | 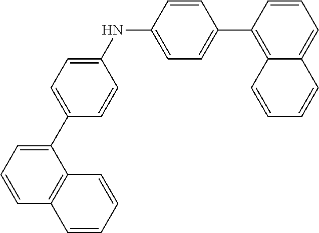 [SMILES: c1ccc2c(-c3ccc(Nc4ccc(-c5cccc6ccccc56)cc4)cc3)cccc2c1] |
| 189 | | 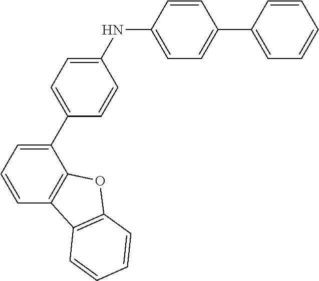 [SMILES: c1ccc(-c2ccc(Nc3ccc(-c4cccc5c4oc4ccccc45)cc3)cc2)cc1] |
| 190 | | 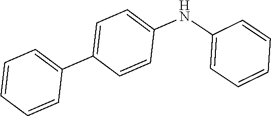 [SMILES: c1ccc(Nc2ccc(-c3ccccc3)cc2)cc1] |
| 191 | | 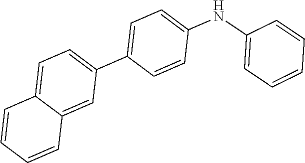 [SMILES: c1ccc(Nc2ccc(-c3ccc4ccccc4c3)cc2)cc1] |
| 192 | | 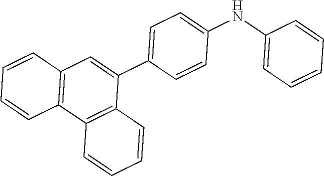 [SMILES: c1ccc(Nc2ccc(-c3cc4ccccc4c4ccccc34)cc2)cc1] |
| 193 | | 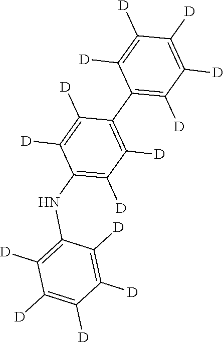 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] |
| 194 | | 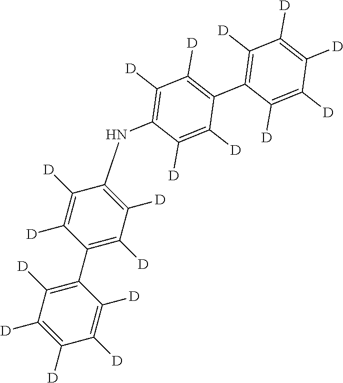 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] |
| 195 | | 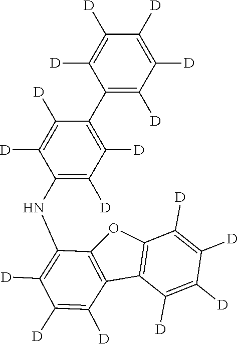 [SMILES: [2H]c1c([2H])c([2H])c(-c2c([2H])c([2H])c(Nc3c([2H])c([2H])c([2H])c4c3oc3c([2H])c([2H])c([2H])c([2H])c34)c([2H])c2[2H])c([2H])c1[2H]] |
| 196 | 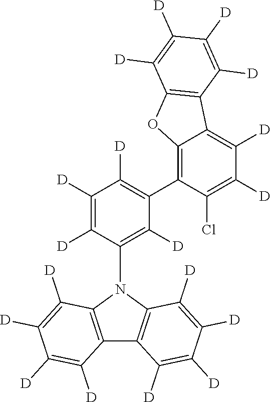 [SMILES: [2H]c1c([2H])c(-c2c(Cl)c([2H])c([2H])c3c2oc2c([2H])c([2H])c([2H])c([2H])c23)c([2H])c(-n2c3c([2H])c([2H])c([2H])c([2H])c3c3c([2H])c([2H])c([2H])c([2H])c32)c1[2H]] | 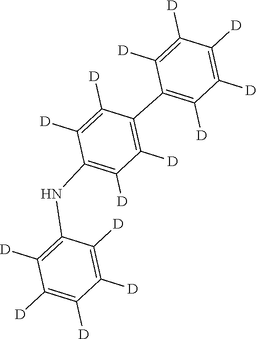 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] |
| 197 | | 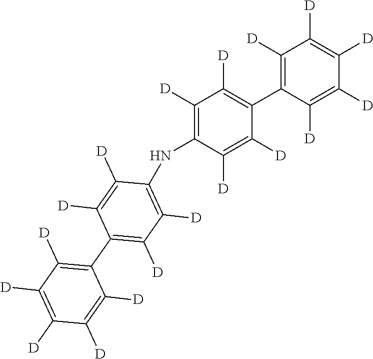 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] |
| 198 | | 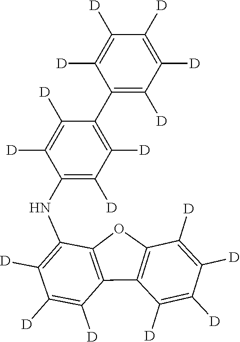 [SMILES: [2H]c1c([2H])c([2H])c(-c2c([2H])c([2H])c(Nc3c([2H])c([2H])c([2H])c4c3oc3c([2H])c([2H])c([2H])c([2H])c34)c([2H])c2[2H])c([2H])c1[2H]] |

| | | Obtained | |
|---|---|---|---|
| Compound | Product | amount | [M + H]⁺ |
| 184 | 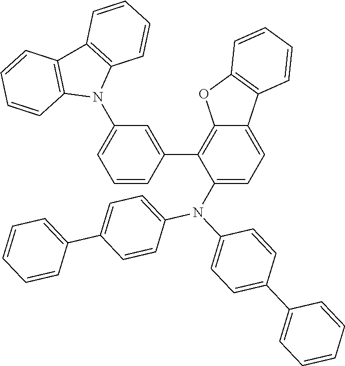 [SMILES: c1ccc(-c2ccc(N(c3ccc(-c4ccccc4)cc3)c3ccc4c(oc5ccccc54)c3-c3cccc(-n4c5ccccc5c5ccccc54)c3)cc2)cc1] | 8.2 g (72%) | 729.29 |
| 185 | 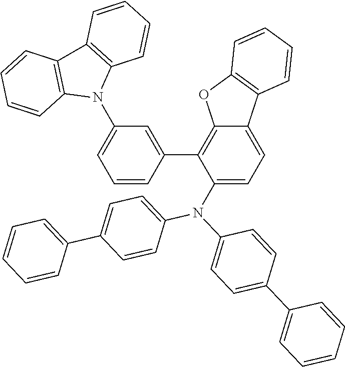 [SMILES: c1ccc(-c2ccc(N(c3ccc(-c4ccccc4)cc3)c3ccc4c(oc5ccccc54)c3-c3cccc(-n4c5ccccc5c5ccccc54)c3)cc2)cc1] | 8.6 g (71%) | 778.30 |
| 186 | 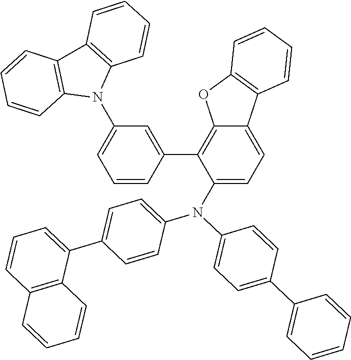 [SMILES: c1ccc(-c2ccc(N(c3ccc(-c4cccc5ccccc45)cc3)c3ccc4c(oc5ccccc54)c3-c3cccc(-n4c5ccccc5c5ccccc54)c3)cc2)cc1] | 8.1 g (67%) | 778.30 |
| 187 | 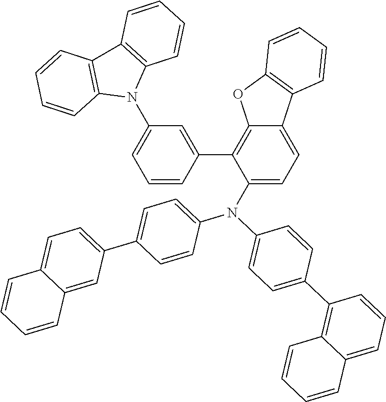 [SMILES: c1cc(-c2c(N(c3ccc(-c4ccc5ccccc5c4)cc3)c3ccc(-c4cccc5ccccc45)cc3)ccc3c2oc2ccccc23)cc(-n2c3ccccc3c3ccccc32)c1] | 8.4 g (65%) | 828.31 |
| 188 | 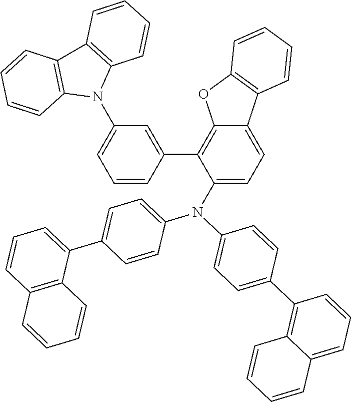 [SMILES: c1cc(-c2c(N(c3ccc(-c4cccc5ccccc45)cc3)c3ccc(-c4cccc5ccccc45)cc3)ccc3c2oc2ccccc23)cc(-n2c3ccccc3c3ccccc32)c1] | 8.0 g (62%) | 828.31 |
| 189 | 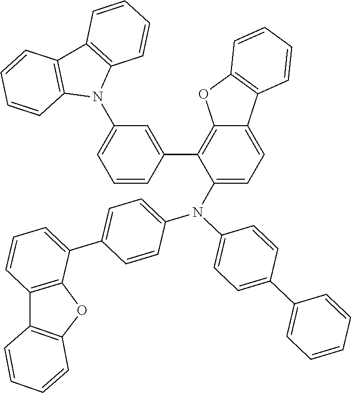 [SMILES: c1ccc(-c2ccc(N(c3ccc(-c4cccc5c4oc4ccccc45)cc3)c3ccc4c(oc5ccccc54)c3-c3cccc(-n4c5ccccc5c5ccccc54)c3)cc2)cc1] | 8.5 g (67%) | 818.29 |
| 190 | 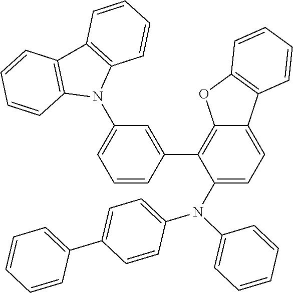 [SMILES: c1ccc(-c2ccc(N(c3ccccc3)c3ccc4c(oc5ccccc54)c3-c3cccc(-n4c5ccccc5c5ccccc54)c3)cc2)cc1] | 7.7 g (76%) | 652.25 |
| 191 | 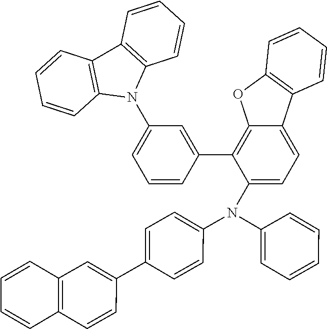 [SMILES: c1ccc(N(c2ccc(-c3ccc4ccccc4c3)cc2)c2ccc3c(oc4ccccc43)c2-c2cccc(-n3c4ccccc4c4ccccc43)c2)cc1] | 8.1 g (74%) | 702.27 |
| 192 | 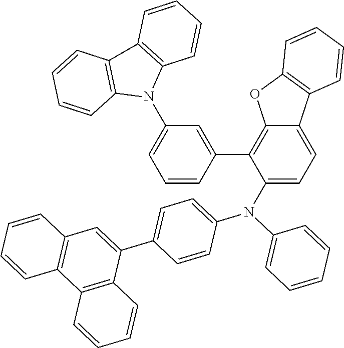 [SMILES: c1ccc(N(c2ccc(-c3cc4ccccc4c4ccccc34)cc2)c2ccc3c(oc4ccccc43)c2-c2cccc(-n3c4ccccc4c4ccccc43)c2)cc1] | 8.4 g (72%) | 752.28 |
| 193 | 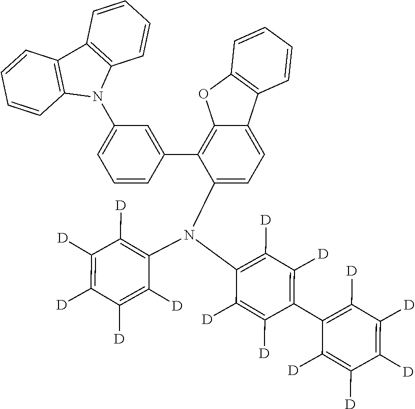 [SMILES: [2H]c1c([2H])c([2H])c(-c2c([2H])c([2H])c(N(c3ccc4c(oc5ccccc54)c3-c3cccc(-n4c5ccccc5c5ccccc54)c3)c3c([2H])c([2H])c([2H])c([2H])c3[2H])c([2H])c2[2H])c([2H])c1[2H]] | 7.7 g (74%) | 666.34 |
| 194 | 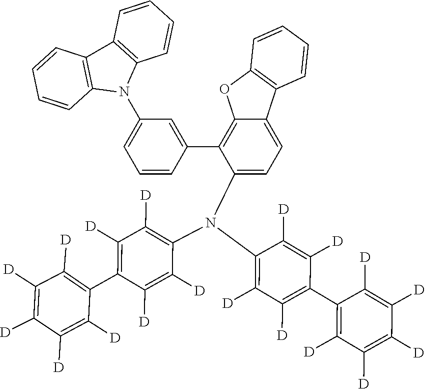 [SMILES: [2H]c1c([2H])c([2H])c(-c2c([2H])c([2H])c(N(c3ccc4c(oc5ccccc54)c3-c3cccc(-n4c5ccccc5c5ccccc54)c3)c3c([2H])c([2H])c(-c4c([2H])c([2H])c([2H])c([2H])c4[2H])c([2H])c3[2H])c([2H])c2[2H])c([2H])c1[2H]] | 8.7 g (75%) | 746.40 |
| 195 | 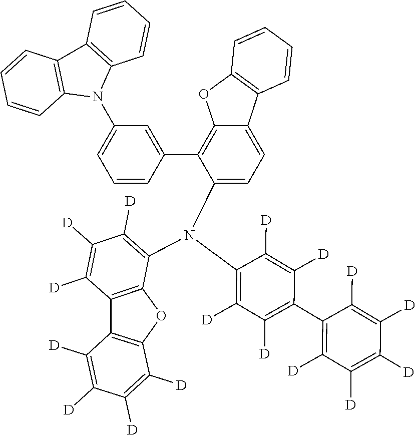 [SMILES: [2H]c1c([2H])c([2H])c(-c2c([2H])c([2H])c(N(c3ccc4c(oc5ccccc54)c3-c3cccc(-n4c5ccccc5c5ccccc54)c3)c3c([2H])c([2H])c([2H])c4c3oc3c([2H])c([2H])c([2H])c([2H])c34)c([2H])c2[2H])c([2H])c1[2H]] | 8.4 g (71%) | 758.36 |
| 196 | 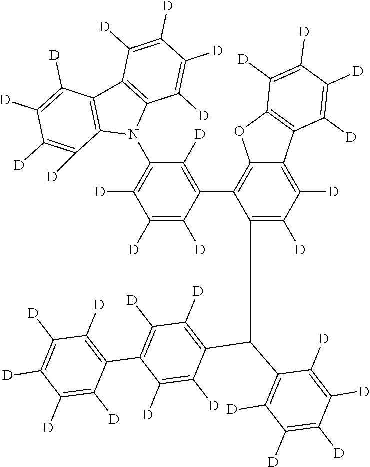 [SMILES: [2H]c1c([2H])c([2H])c(-c2c([2H])c([2H])c(C(c3c([2H])c([2H])c([2H])c([2H])c3[2H])c3c([2H])c([2H])c4c(oc5c([2H])c([2H])c([2H])c([2H])c54)c3-c3c([2H])c([2H])c([2H])c(-n4c5c([2H])c([2H])c([2H])c([2H])c5c5c([2H])c([2H])c([2H])c([2H])c54)c3[2H])c([2H])c2[2H])c([2H])c1[2H]] | 7.5 g (70%) | 684.45 |
| 197 | 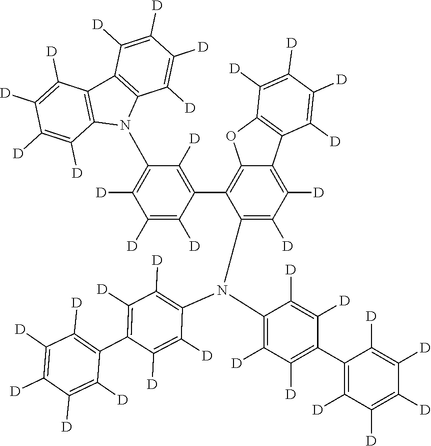 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4c(oc5c([2H])c([2H])c([2H])c([2H])c54)c3-c3c([2H])c([2H])c([2H])c(-n4c5c([2H])c([2H])c([2H])c([2H])c5c5c([2H])c([2H])c([2H])c([2H])c54)c3[2H])c([2H])c2[2H])c([2H])c1[2H]] | 8.1 g (68%) | 764.51 |
| 198 | 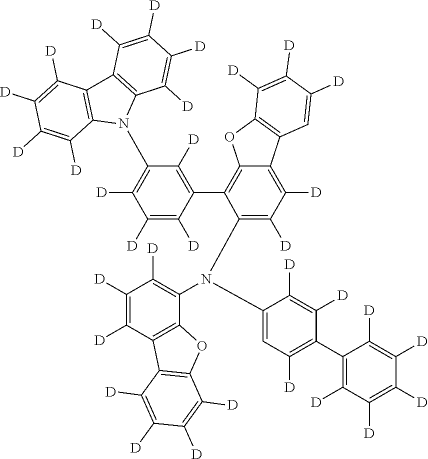 [SMILES: [2H]c1cc2c(oc3c(-c4c([2H])c([2H])c([2H])c(-n5c6c([2H])c([2H])c([2H])c([2H])c6c6c([2H])c([2H])c([2H])c([2H])c65)c4[2H])c(N(c4cc([2H])c(-c5c([2H])c([2H])c([2H])c([2H])c5[2H])c([2H])c4[2H])c4c([2H])c([2H])c([2H])c5c4oc4c([2H])c([2H])c([2H])c([2H])c45)c([2H])c([2H])c32)c([2H])c1[2H]] | 9.2 g (76%) | 776.48 |

TABLE 17

| | SUB 2-29 | |
|---|---|---|
| Compound | SUB 2-35 | SUB 1 |
| 199 | 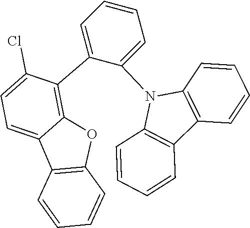 [SMILES: Clc1ccc2c(oc3ccccc32)c1-c1ccccc1-n1c2ccccc2c2ccccc21] | 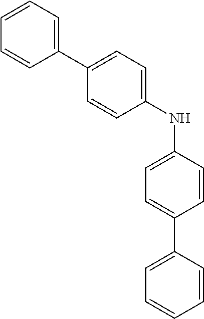 [SMILES: c1ccc(-c2ccc(Nc3ccc(-c4ccccc4)cc3)cc2)cc1] |
| 200 | | 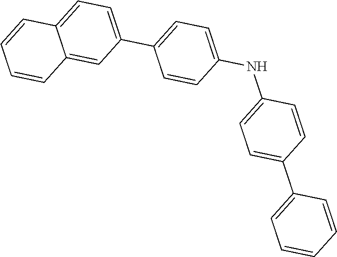 [SMILES: c1ccc(-c2ccc(Nc3ccc(-c4ccc5ccccc5c4)cc3)cc2)cc1] |
| 201 | | 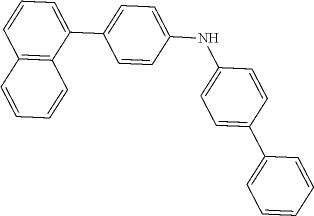 [SMILES: c1ccc(-c2ccc(Nc3ccc(-c4cccc5ccccc45)cc3)cc2)cc1] |
| 202 | | 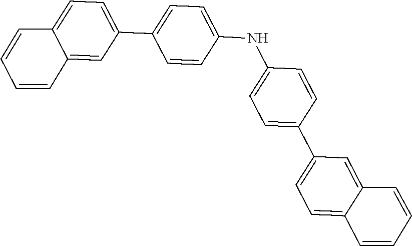 [SMILES: c1ccc2cc(-c3ccc(Nc4ccc(-c5ccc6ccccc6c5)cc4)cc3)ccc2c1] |
| 203 | | 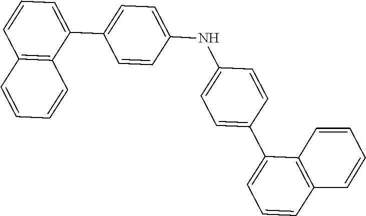 [SMILES: c1ccc2c(-c3ccc(Nc4ccc(-c5cccc6ccccc56)cc4)cc3)cccc2c1] |
| 204 | | 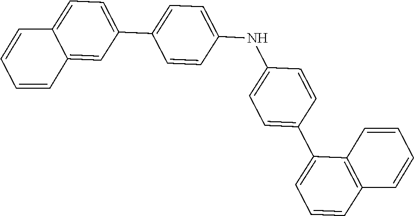 [SMILES: c1ccc2cc(-c3ccc(Nc4ccc(-c5cccc6ccccc56)cc4)cc3)ccc2c1] |
| 205 | | 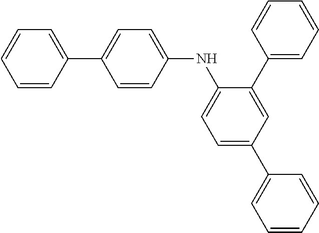 [SMILES: c1ccc(-c2ccc(Nc3ccc(-c4ccccc4)cc3-c3ccccc3)cc2)cc1] |
| 206 | | 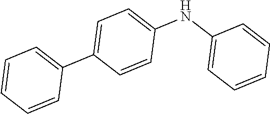 [SMILES: c1ccc(Nc2ccc(-c3ccccc3)cc2)cc1] |
| 207 | | 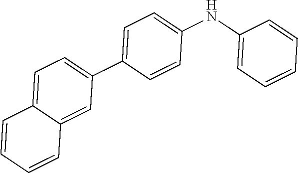 [SMILES: c1ccc(Nc2ccc(-c3ccc4ccccc4c3)cc2)cc1] |
| 208 | | 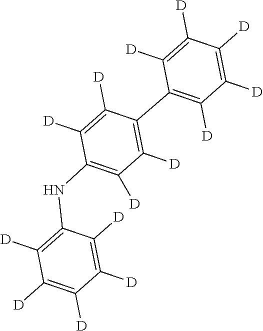 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] |
| 209 | | 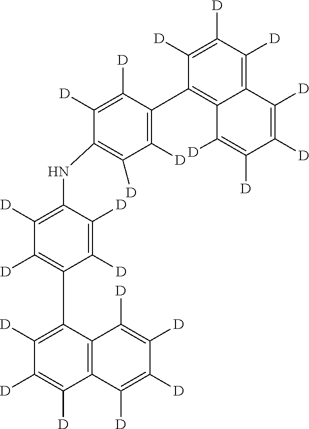 [SMILES: [2H]c1c([2H])c(-c2c([2H])c([2H])c([2H])c3c([2H])c([2H])c([2H])c([2H])c23)c([2H])c([2H])c1Nc1c([2H])c([2H])c(-c2c([2H])c([2H])c([2H])c3c([2H])c([2H])c([2H])c([2H])c23)c([2H])c1[2H]] |
| 210 | | 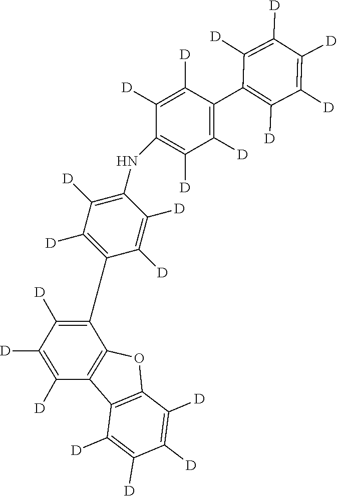 [SMILES: [2H]c1c([2H])c([2H])c(-c2c([2H])c([2H])c(Nc3c([2H])c([2H])c(-c4c([2H])c([2H])c([2H])c5c4oc4c([2H])c([2H])c([2H])c([2H])c45)c([2H])c3[2H])c([2H])c2[2H])c([2H])c1[2H]] |
| 211 | 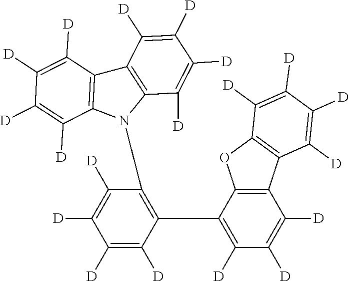 [SMILES: [2H]c1c([2H])c([2H])c(-n2c3c([2H])c([2H])c([2H])c([2H])c3c3c([2H])c([2H])c([2H])c([2H])c32)c(-c2c([2H])c([2H])c([2H])c3c2oc2c([2H])c([2H])c([2H])c([2H])c23)c1[2H]] | 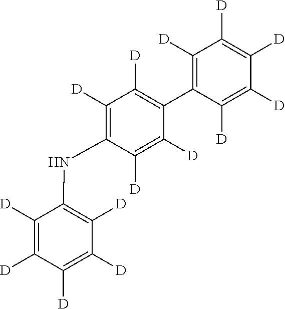 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] |
| 212 | | 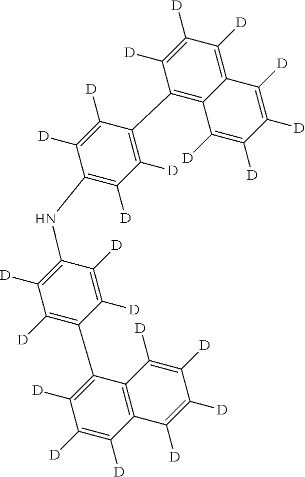 [SMILES: [2H]c1c([2H])c(-c2c([2H])c([2H])c([2H])c3c([2H])c([2H])c([2H])c([2H])c23)c([2H])c([2H])c1Nc1c([2H])c([2H])c(-c2c([2H])c([2H])c([2H])c3c([2H])c([2H])c([2H])c([2H])c23)c([2H])c1[2H]] |
| 213 | | 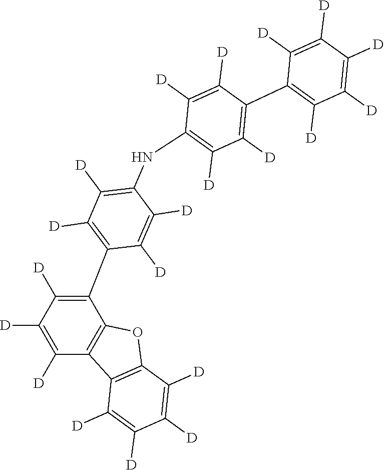 [SMILES: [2H]c1c([2H])c([2H])c(-c2c([2H])c([2H])c(Nc3c([2H])c([2H])c(-c4c([2H])c([2H])c([2H])c5c4oc4c([2H])c([2H])c([2H])c([2H])c45)c([2H])c3[2H])c([2H])c2[2H])c([2H])c1[2H]] |

| | | Obtained | |
|---|---|---|---|
| Compound | Product | amount | [M + H]⁺ |
| 199 | 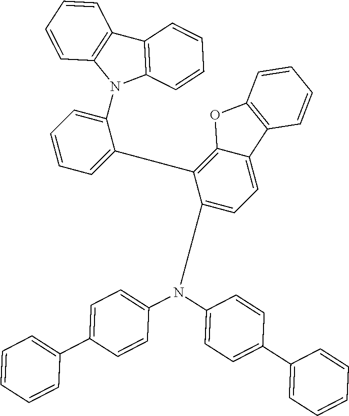 [SMILES: c1ccc(-c2ccc(N(c3ccc(-c4ccccc4)cc3)c3ccc4c(oc5ccccc54)c3-c3ccccc3-n3c4ccccc4c4ccccc43)cc2)cc1] | 8.0 g (71%) | 728.28 |
| 200 | 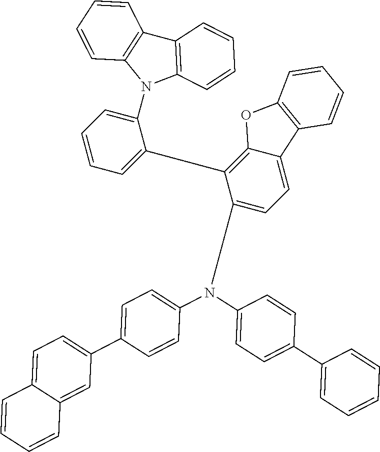 [SMILES: c1ccc(-c2ccc(N(c3ccc(-c4ccc5ccccc5c4)cc3)c3ccc4c(oc5ccccc54)c3-c3ccccc3-n3c4ccccc4c4ccccc43)cc2)cc1] | 9.1 g (75%) | 778.30 |
| 201 | 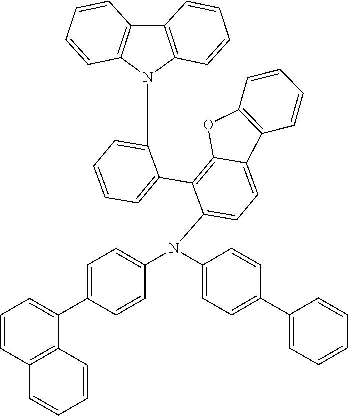 [SMILES: c1ccc(-c2ccc(N(c3ccc(-c4cccc5ccccc45)cc3)c3ccc4c(oc5ccccc54)c3-c3ccccc3-n3c4ccccc4c4ccccc43)cc2)cc1] | 9.0 g (74%) | 778.30 |
| 202 | 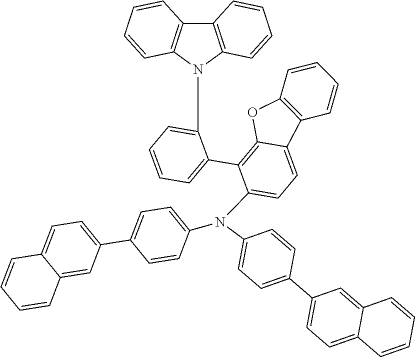 [SMILES: c1ccc(-n2c3ccccc3c3ccccc32)c(-c2c(N(c3ccc(-c4ccc5ccccc5c4)cc3)c3ccc(-c4ccc5ccccc5c4)cc3)ccc3c2oc2ccccc23)c1] | 9.0 g (70%) | 828.31 |
| 203 | 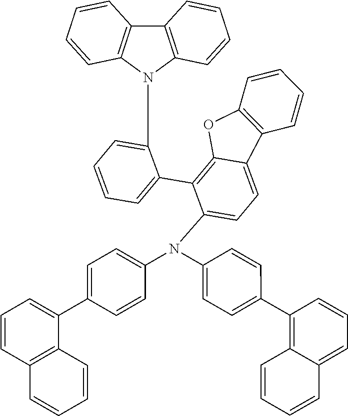 [SMILES: c1ccc(-n2c3ccccc3c3ccccc32)c(-c2c(N(c3ccc(-c4cccc5ccccc45)cc3)c3ccc(-c4cccc5ccccc45)cc3)ccc3c2oc2ccccc23)c1] | 9.3 g (72%) | 828.31 |
| 204 | 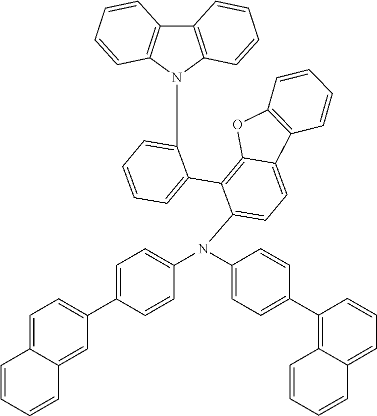 [SMILES: c1ccc(-n2c3ccccc3c3ccccc32)c(-c2c(N(c3ccc(-c4ccc5ccccc5c4)cc3)c3ccc(-c4cccc5ccccc45)cc3)ccc3c2oc2ccccc23)c1] | 8.8 g (68%) | 828.31 |
| 205 | 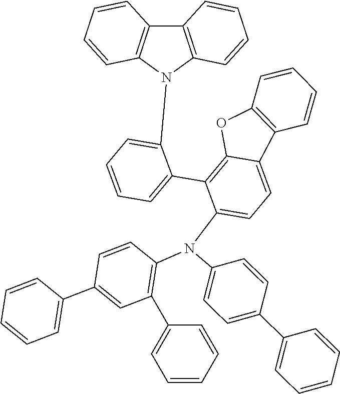 [SMILES: c1ccc(-c2ccc(N(c3ccc(-c4ccccc4)cc3-c3ccccc3)c3ccc4c(oc5ccccc54)c3-c3ccccc3-n3c4ccccc4c4ccccc43)cc2)cc1] | 8.4 g (67%) | 804.31 |
| 206 | 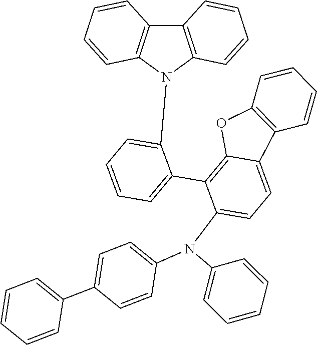 [SMILES: c1ccc(-c2ccc(N(c3ccccc3)c3ccc4c(oc5ccccc54)c3-c3ccccc3-n3c4ccccc4c4ccccc43)cc2)cc1] | 7.2 g (71%) | 652.25 |
| 207 | 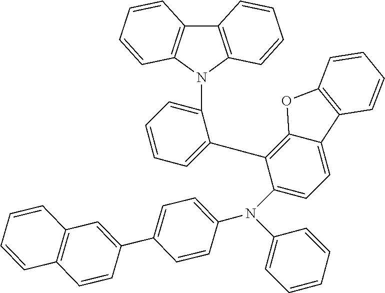 [SMILES: c1ccc(N(c2ccc(-c3ccc4ccccc4c3)cc2)c2ccc3c(oc4ccccc43)c2-c2ccccc2-n2c3ccccc3c3ccccc32)cc1] | 8.3 g (76%) | 702.27 |
| 208 | 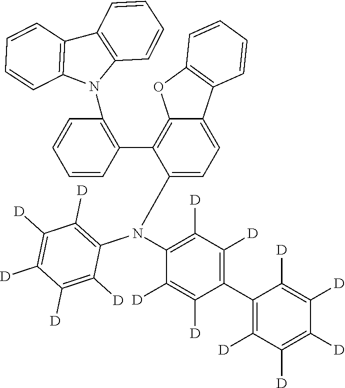 [SMILES: [2H]c1c([2H])c([2H])c(-c2c([2H])c([2H])c(N(c3ccc4c(oc5ccccc54)c3-c3ccccc3-n3c4ccccc4c4ccccc43)c3c([2H])c([2H])c([2H])c([2H])c3[2H])c([2H])c2[2H])c([2H])c1[2H]] | 7.5 g (72%) | 666.34 |
| 209 | 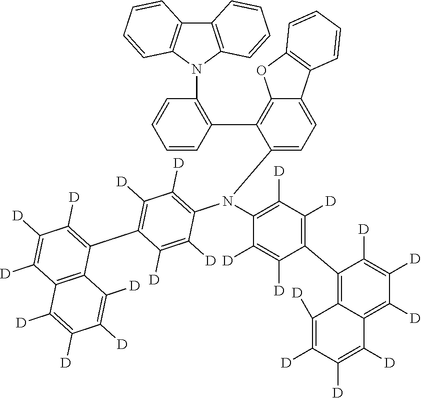 [SMILES: [2H]c1c([2H])c(N(c2ccc3c(oc4ccccc43)c2-c2ccccc2-n2c3ccccc3c3ccccc32)c2c([2H])c([2H])c(-c3c([2H])c([2H])c([2H])c4c([2H])c([2H])c([2H])c([2H])c34)c([2H])c2[2H])c([2H])c([2H])c1-c1c([2H])c([2H])c([2H])c2c([2H])c([2H])c([2H])c([2H])c12] | 10.7 g (81%) | 850.45 |
| 210 | 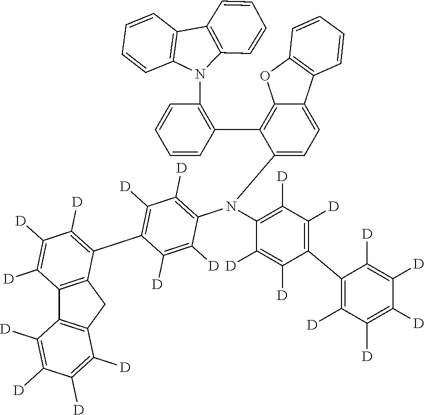 [SMILES: [2H]c1c([2H])c([2H])c(-c2c([2H])c([2H])c(N(c3ccc4c(oc5ccccc54)c3-c3ccccc3-n3c4ccccc4c4ccccc43)c3c([2H])c([2H])c(-c4c([2H])c([2H])c([2H])c5c4Cc4c([2H])c([2H])c([2H])c([2H])c4-5)c([2H])c3[2H])c([2H])c2[2H])c([2H])c1[2H]] | 9.7 g (74%) | 838.42 |
| 211 | 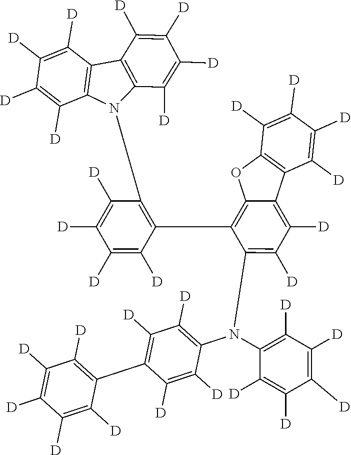 [SMILES: [2H]c1c([2H])c([2H])c(-c2c([2H])c([2H])c(N(c3c([2H])c([2H])c([2H])c([2H])c3[2H])c3c([2H])c([2H])c4c(oc5c([2H])c([2H])c([2H])c([2H])c54)c3-c3c([2H])c([2H])c([2H])c([2H])c3-n3c4c([2H])c([2H])c([2H])c([2H])c4c4c([2H])c([2H])c([2H])c([2H])c43)c([2H])c2[2H])c([2H])c1[2H]] | 8.7 g (82%) | 684.45 |
| 212 | 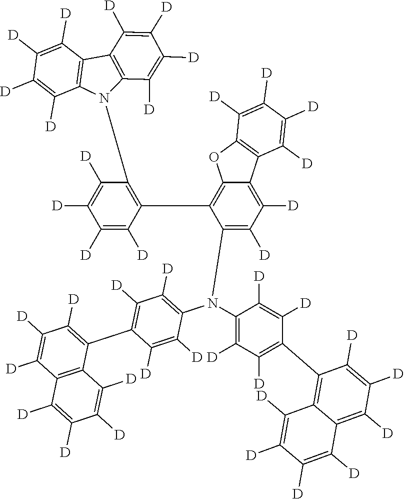 [SMILES: [2H]c1c([2H])c([2H])c(-n2c3c([2H])c([2H])c([2H])c([2H])c3c3c([2H])c([2H])c([2H])c([2H])c32)c(-c2c(N(c3c([2H])c([2H])c(-c4c([2H])c([2H])c([2H])c5c([2H])c([2H])c([2H])c([2H])c45)c([2H])c3[2H])c3c([2H])c([2H])c(-c4c([2H])c([2H])c([2H])c5c([2H])c([2H])c([2H])c([2H])c45)c([2H])c3[2H])c([2H])c([2H])c3c2oc2c([2H])c([2H])c([2H])c([2H])c23)c1[2H]] | 11.1 g (82%) | 868.57 |
| 213 | 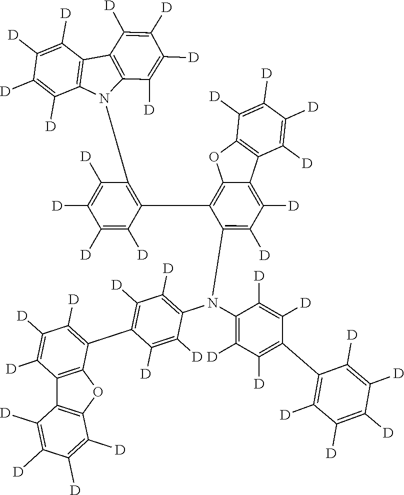 [SMILES: [2H]c1c([2H])c([2H])c(-c2c([2H])c([2H])c(N(c3c([2H])c([2H])c(-c4c([2H])c([2H])c([2H])c5c4oc4c([2H])c([2H])c([2H])c([2H])c45)c([2H])c3[2H])c3c([2H])c([2H])c4c(oc5c([2H])c([2H])c([2H])c([2H])c54)c3-c3c([2H])c([2H])c([2H])c([2H])c3-n3c4c([2H])c([2H])c([2H])c([2H])c4c4c([2H])c([2H])c([2H])c([2H])c43)c([2H])c2[2H])c([2H])c1[2H]] | 9.7 g (73%) | 856.53 |

TABLE 18

| | SUB 2-26 | |
|---|---|---|
| Compound | SUB 2-32 | SUB 1 |
| 214 | 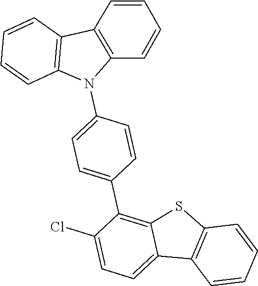 [SMILES: Clc1ccc2c(sc3ccccc32)c1-c1ccc(-n2c3ccccc3c3ccccc32)cc1] | 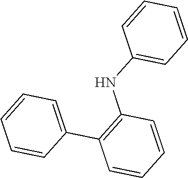 [SMILES: c1ccc(Nc2ccccc2-c2ccccc2)cc1] |
| 215 | | 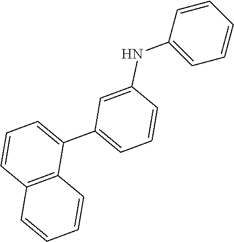 [SMILES: c1ccc(Nc2cccc(-c3cccc4ccccc34)c2)cc1] |
| 216 | | 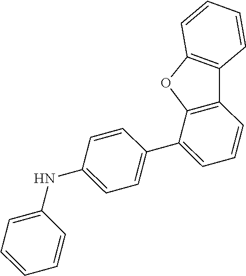 [SMILES: c1ccc(Nc2ccc(-c3cccc4c3oc3ccccc34)cc2)cc1] |
| 217 | | 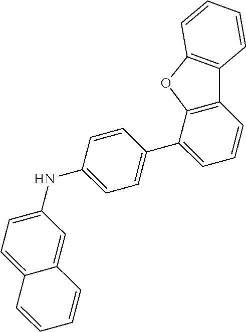 [SMILES: c1ccc2cc(Nc3ccc(-c4cccc5c4oc4ccccc45)cc3)ccc2c1] |
| 218 | | 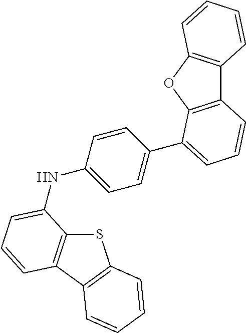 [SMILES: c1ccc2c(c1)oc1c(-c3ccc(Nc4cccc5c4sc4ccccc45)cc3)cccc12] |
| 219 | | 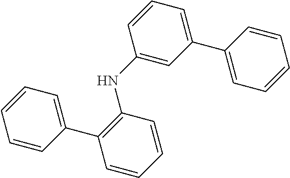 [SMILES: c1ccc(-c2cccc(Nc3ccccc3-c3ccccc3)c2)cc1] |
| 220 | | 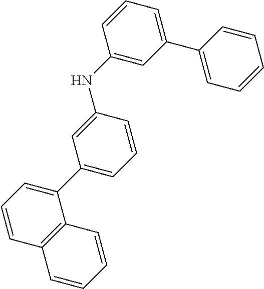 [SMILES: c1ccc(-c2cccc(Nc3cccc(-c4cccc5ccccc45)c3)c2)cc1] |
| 221 | | 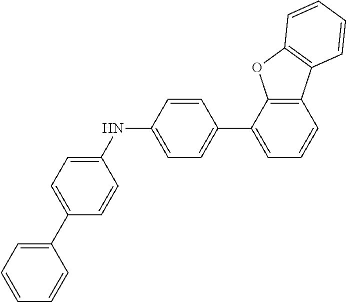 [SMILES: c1ccc(-c2ccc(Nc3ccc(-c4cccc5c4oc4ccccc45)cc3)cc2)cc1] |
| 222 | | 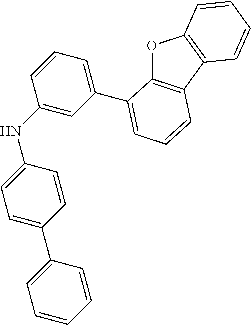 [SMILES: c1ccc(-c2ccc(Nc3cccc(-c4cccc5c4oc4ccccc45)c3)cc2)cc1] |
| 223 | | 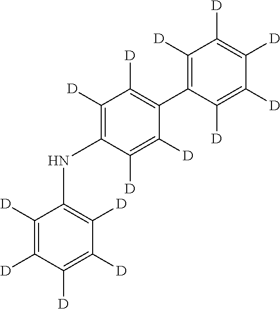 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] |
| 224 | | 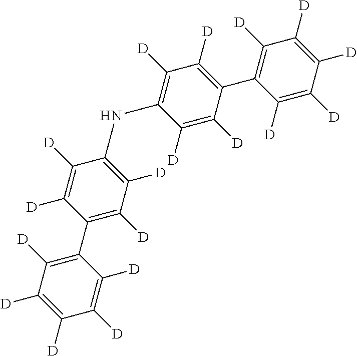 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] |
| 225 | 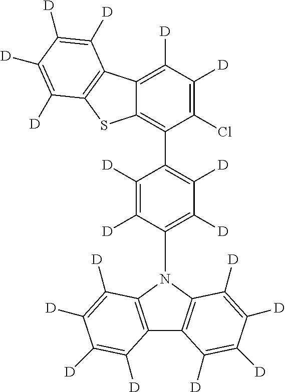 [SMILES: [2H]c1c([2H])c(-n2c3c([2H])c([2H])c([2H])c([2H])c3c3c([2H])c([2H])c([2H])c([2H])c32)c([2H])c([2H])c1-c1c(Cl)c([2H])c([2H])c2c1sc1c([2H])c([2H])c([2H])c([2H])c12] | 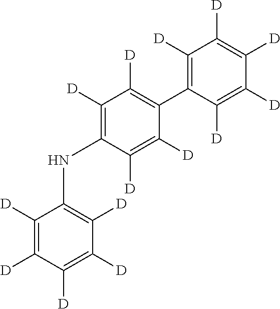 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] |
| 226 | | 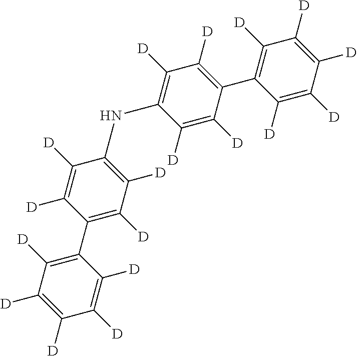 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] |

| | | Obtained | |
|---|---|---|---|
| Compound | Product | amount | [M + H]⁺ |
| 214 | 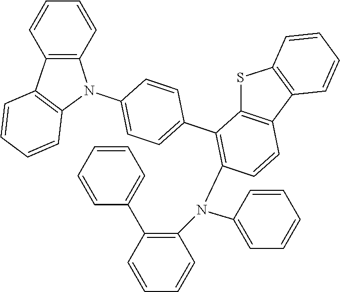 [SMILES: c1ccc(-c2ccccc2N(c2ccccc2)c2ccc3c(sc4ccccc43)c2-c2ccc(-n3c4ccccc4c4ccccc43)cc2)cc1] | 7.3 g (70%) | 668.23 |
| 215 | 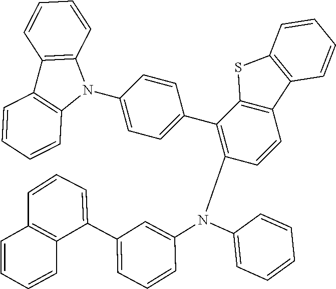 [SMILES: c1ccc(N(c2cccc(-c3cccc4ccccc34)c2)c2ccc3c(sc4ccccc43)c2-c2ccc(-n3c4ccccc4c4ccccc43)cc2)cc1] | 7.5 g (67%) | 718.24 |
| 216 | 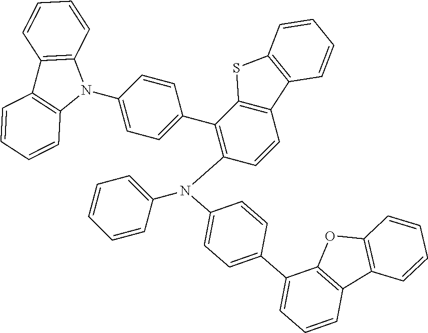 [SMILES: c1ccc(N(c2ccc(-c3cccc4c3oc3ccccc34)cc2)c2ccc3c(sc4ccccc43)c2-c2ccc(-n3c4ccccc4c4ccccc43)cc2)cc1] | 7.9 g (67%) | 758.24 |
| 217 | 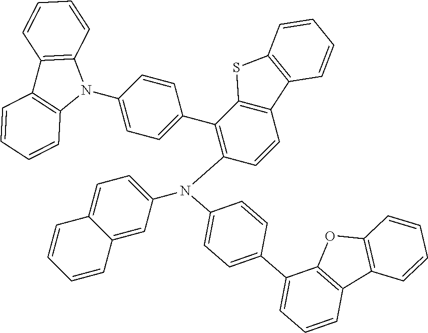 [SMILES: c1ccc2cc(N(c3ccc(-c4cccc5c4oc4ccccc45)cc3)c3ccc4c(sc5ccccc54)c3-c3ccc(-n4c5ccccc5c5ccccc54)cc3)ccc2c1] | 8.6 g (68%) | 808.25 |
| 218 | 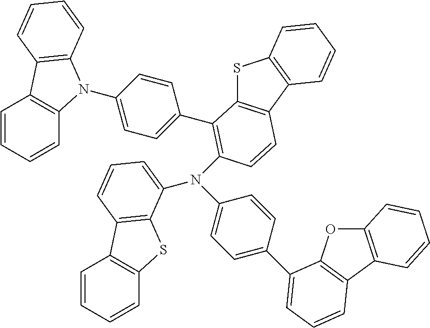 [SMILES: c1ccc2c(c1)oc1c(-c3ccc(N(c4ccc5c(sc6ccccc65)c4-c4ccc(-n5c6ccccc6c6ccccc65)cc4)c4cccc5c4sc4ccccc45)cc3)cccc12] | 8.2 g (61%) | 864.23 |
| 219 | 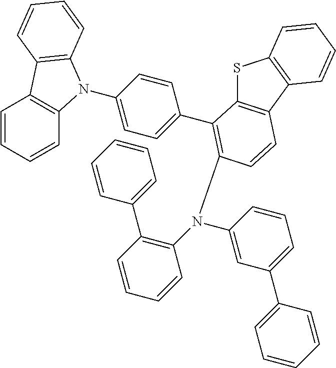 [SMILES: c1ccc(-c2cccc(N(c3ccccc3-c3ccccc3)c3ccc4c(sc5ccccc54)c3-c3ccc(-n4c5ccccc5c5ccccc54)cc3)c2)cc1] | 7.2 g (62%) | 744.26 |
| 220 | 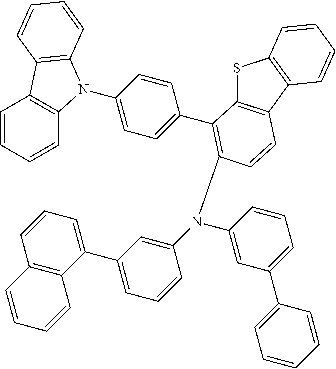 [SMILES: c1ccc(-c2cccc(N(c3cccc(-c4cccc5ccccc45)c3)c3ccc4c(sc5ccccc54)c3-c3ccc(-n4c5ccccc5c5ccccc54)cc3)c2)cc1] | 8.5 g (69%) | 794.28 |
| 221 | 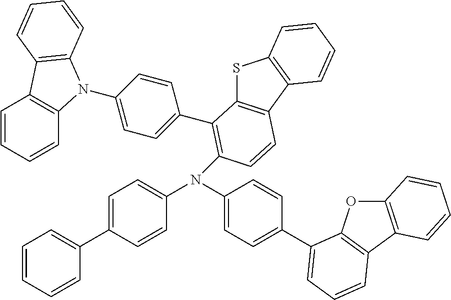 [SMILES: c1ccc(-c2ccc(N(c3ccc(-c4cccc5c4oc4ccccc45)cc3)c3ccc4c(sc5ccccc54)c3-c3ccc(-n4c5ccccc5c5ccccc54)cc3)cc2)cc1] | 9.2 g (71%) | 834.27 |
| 222 | 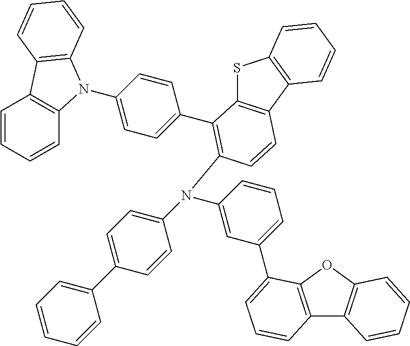 [SMILES: c1ccc(-c2ccc(N(c3cccc(-c4cccc5c4oc4ccccc45)c3)c3ccc4c(sc5ccccc54)c3-c3ccc(-n4c5ccccc5c5ccccc54)cc3)cc2)cc1] | 9.1 g (70%) | 834.27 |
| 223 | 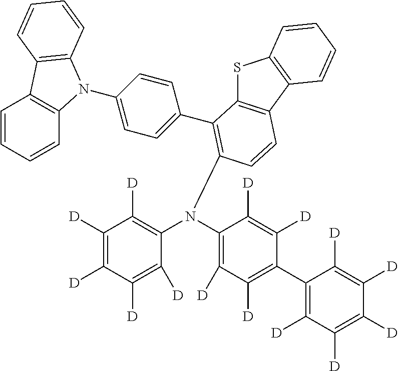 [SMILES: [2H]c1c([2H])c([2H])c(-c2c([2H])c([2H])c(N(c3ccc4c(sc5ccccc54)c3-c3ccc(-n4c5ccccc5c5ccccc54)cc3)c3c([2H])c([2H])c([2H])c([2H])c3[2H])c([2H])c2[2H])c([2H])c1[2H]] | 7.4 g (70%) | 682.32 |
| 224 | 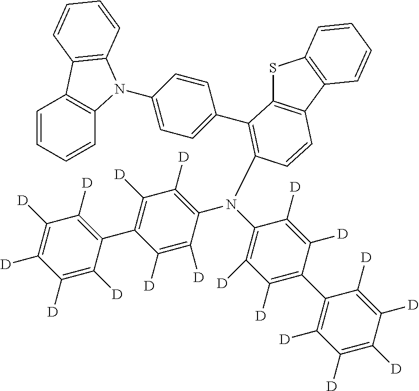 [SMILES: [2H]c1c([2H])c([2H])c(-c2c([2H])c([2H])c(N(c3ccc4c(sc5ccccc54)c3-c3ccc(-n4c5ccccc5c5ccccc54)cc3)c3c([2H])c([2H])c(-c4c([2H])c([2H])c([2H])c([2H])c4[2H])c([2H])c3[2H])c([2H])c2[2H])c([2H])c1[2H]] | 8.1 g (68%) | 762.37 |
| 225 | 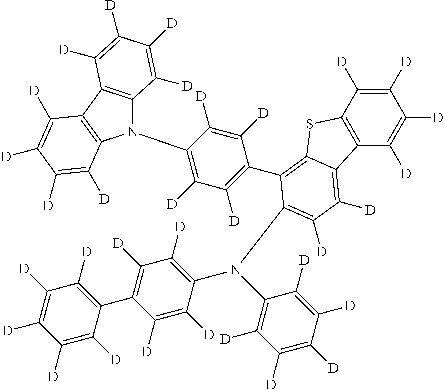 [SMILES: [2H]c1c([2H])c([2H])c(-c2c([2H])c([2H])c(N(c3c([2H])c([2H])c([2H])c([2H])c3[2H])c3c([2H])c([2H])c4c(sc5c([2H])c([2H])c([2H])c([2H])c54)c3-c3c([2H])c([2H])c(-n4c5c([2H])c([2H])c([2H])c([2H])c5c5c([2H])c([2H])c([2H])c([2H])c54)c([2H])c3[2H])c([2H])c2[2H])c([2H])c1[2H]] | 7.0 g (64%) | 700.43 |
| 226 | 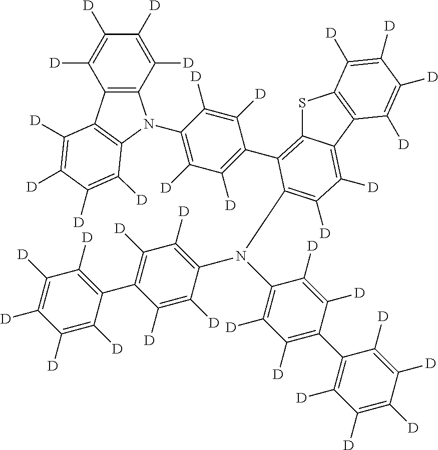 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4c(sc5c([2H])c([2H])c([2H])c([2H])c54)c3-c3c([2H])c([2H])c(-n4c5c([2H])c([2H])c([2H])c([2H])c5c5c([2H])c([2H])c([2H])c([2H])c54)c([2H])c3[2H])c([2H])c2[2H])c([2H])c1[2H]] | 7.4 g (61%) | 780.49 |

TABLE 19

| | SUB 2-28 | |
|---|---|---|
| Compound | SUB 2-34 | SUB 1 |
| 227 | 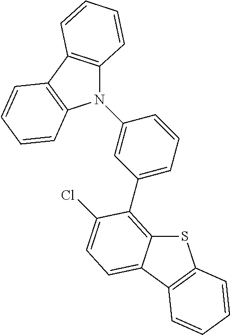 [SMILES: Clc1ccc2c(sc3ccccc32)c1-c1cccc(-n2c3ccccc3c3ccccc32)c1] | 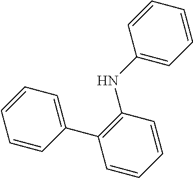 [SMILES: c1ccc(Nc2ccccc2-c2ccccc2)cc1] |
| 228 | | 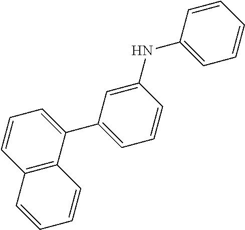 [SMILES: c1ccc(Nc2cccc(-c3cccc4ccccc34)c2)cc1] |
| 229 | | 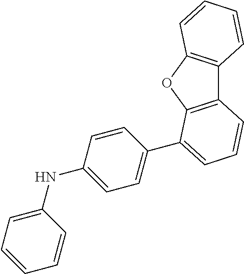 [SMILES: c1ccc(Nc2ccc(-c3cccc4c3oc3ccccc34)cc2)cc1] |
| 230 | | 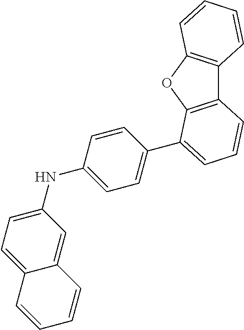 [SMILES: c1ccc2cc(Nc3ccc(-c4cccc5c4oc4ccccc45)cc3)ccc2c1] |
| 231 | | 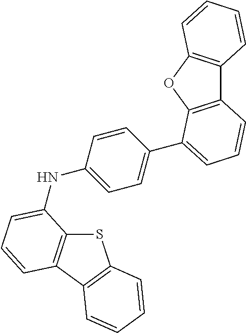 [SMILES: c1ccc2c(c1)oc1c(-c3ccc(Nc4cccc5c4sc4ccccc45)cc3)cccc12] |
| 232 | | 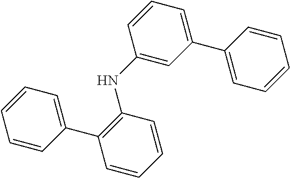 [SMILES: c1ccc(-c2cccc(Nc3ccccc3-c3ccccc3)c2)cc1] |
| 233 | | 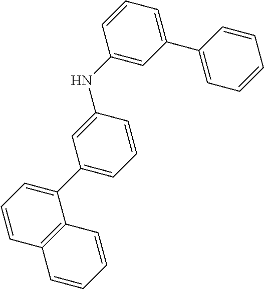 [SMILES: c1ccc(-c2cccc(Nc3cccc(-c4cccc5ccccc45)c3)c2)cc1] |
| 234 | | 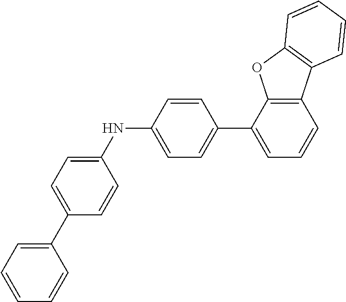 [SMILES: c1ccc(-c2ccc(Nc3ccc(-c4cccc5c4oc4ccccc45)cc3)cc2)cc1] |
| 235 | | 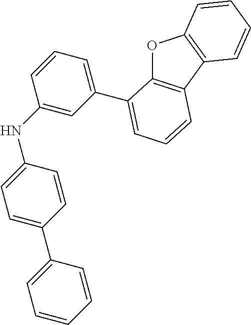 [SMILES: c1ccc(-c2ccc(Nc3cccc(-c4cccc5c4oc4ccccc45)c3)cc2)cc1] |
| 236 | | 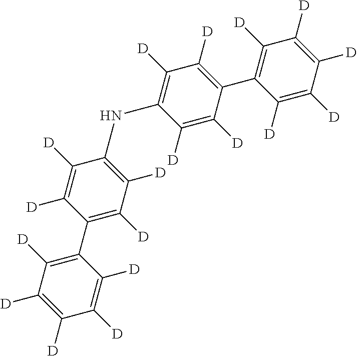 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] |
| 237 | | 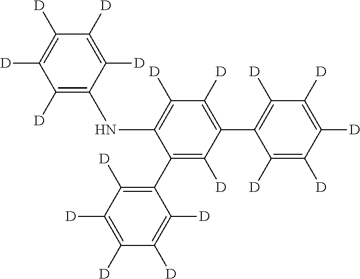 [SMILES: [2H]c1c([2H])c([2H])c(Nc2c([2H])c([2H])c(-c3c([2H])c([2H])c([2H])c([2H])c3[2H])c([2H])c2-c2c([2H])c([2H])c([2H])c([2H])c2[2H])c([2H])c1[2H]] |
| 238 | 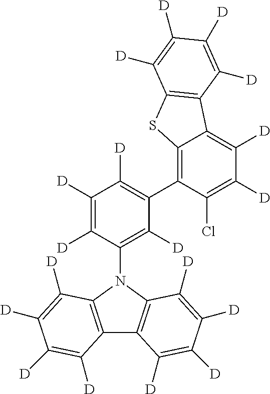 [SMILES: [2H]c1c([2H])c(-c2c(Cl)c([2H])c([2H])c3c2sc2c([2H])c([2H])c([2H])c([2H])c23)c([2H])c(-n2c3c([2H])c([2H])c([2H])c([2H])c3c3c([2H])c([2H])c([2H])c([2H])c32)c1[2H]] | 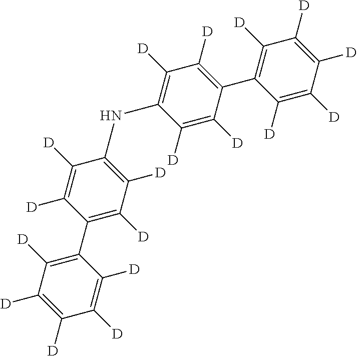 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] |
| 239 | | 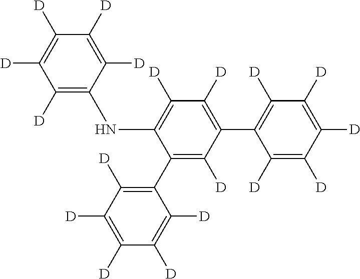 [SMILES: [2H]c1c([2H])c([2H])c(Nc2c([2H])c([2H])c(-c3c([2H])c([2H])c([2H])c([2H])c3[2H])c([2H])c2-c2c([2H])c([2H])c([2H])c([2H])c2[2H])c([2H])c1[2H]] |

| | | Obtained | |
|---|---|---|---|
| Compound | Product | amount | [M + H]⁺ |
| 227 | 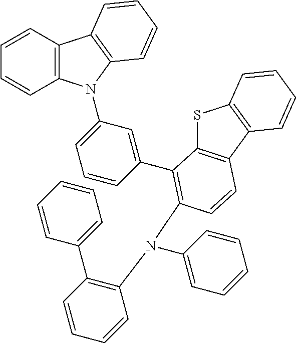 [SMILES: c1ccc(-c2ccccc2N(c2ccccc2)c2ccc3c(sc4ccccc43)c2-c2cccc(-n3c4ccccc4c4ccccc43)c2)cc1] | 7.6 g (73%) | 668.23 |
| 228 | 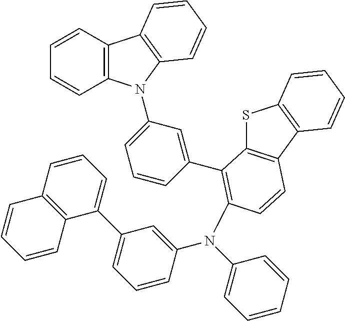 [SMILES: c1ccc(N(c2cccc(-c3cccc4ccccc34)c2)c2ccc3c(sc4ccccc43)c2-c2cccc(-n3c4ccccc4c4ccccc43)c2)cc1] | 8.0 g (72%) | 718.24 |
| 229 | 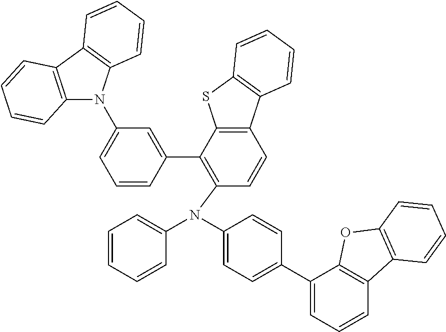 [SMILES: c1ccc(N(c2ccc(-c3cccc4c3oc3ccccc34)cc2)c2ccc3c(sc4ccccc43)c2-c2cccc(-n3c4ccccc4c4ccccc43)c2)cc1] | 9.3 g (79%) | 758.24 |
| 230 | 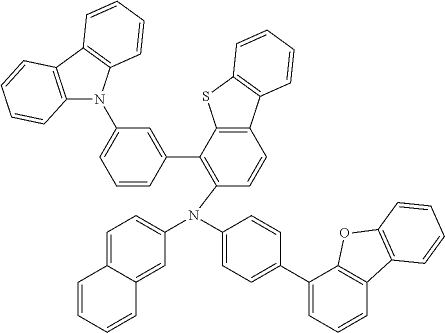 [SMILES: c1cc(-c2c(N(c3ccc(-c4cccc5c4oc4ccccc45)cc3)c3ccc4ccccc4c3)ccc3c2sc2ccccc23)cc(-n2c3ccccc3c3ccccc32)c1] | 10.1 g (80%) | 808.25 |
| 231 | 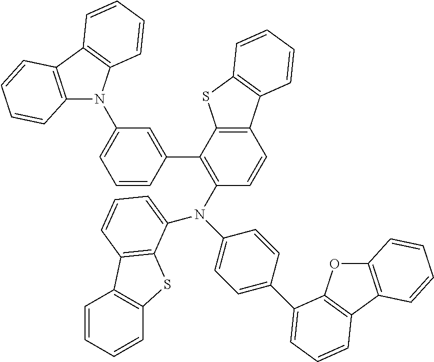 [SMILES: c1cc(-c2c(N(c3ccc(-c4cccc5c4oc4ccccc45)cc3)c3cccc4c3sc3ccccc34)ccc3c2sc2ccccc23)cc(-n2c3ccccc3c3ccccc32)c1] | 10.8 g (80%) | 864.23 |
| 232 | 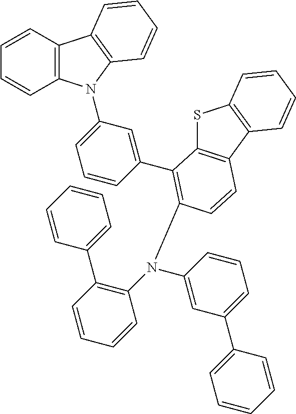 [SMILES: c1ccc(-c2cccc(N(c3ccccc3-c3ccccc3)c3ccc4c(sc5ccccc54)c3-c3cccc(-n4c5ccccc5c5ccccc54)c3)c2)cc1] | 8.1 g (70%) | 744.26 |
| 233 | 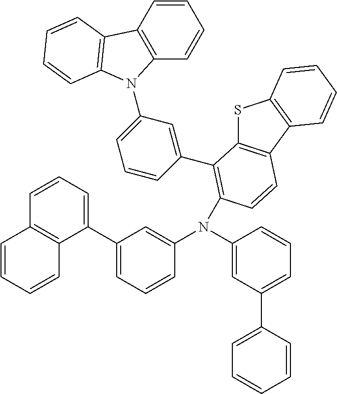 [SMILES: c1ccc(-c2cccc(N(c3cccc(-c4cccc5ccccc45)c3)c3ccc4c(sc5ccccc54)c3-c3cccc(-n4c5ccccc5c5ccccc54)c3)c2)cc1] | 8.3 g (67%) | 794.28 |
| 234 | 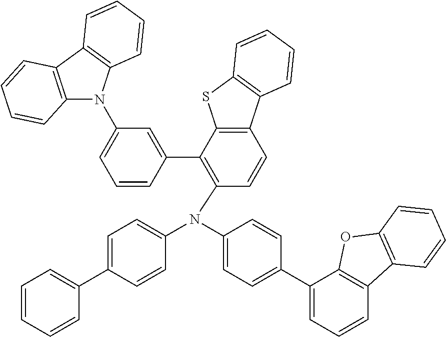 [SMILES: c1ccc(-c2ccc(N(c3ccc(-c4cccc5c4oc4ccccc45)cc3)c3ccc4c(sc5ccccc54)c3-c3cccc(-n4c5ccccc5c5ccccc54)c3)cc2)cc1] | 8.0 g (62%) | 834.27 |
| 235 | 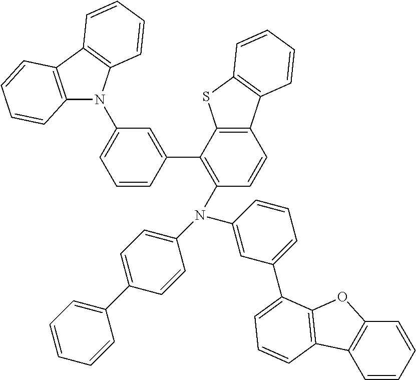 [SMILES: c1ccc(-c2ccc(N(c3cccc(-c4cccc5c4oc4ccccc45)c3)c3ccc4c(sc5ccccc54)c3-c3cccc(-n4c5ccccc5c5ccccc54)c3)cc2)cc1] | 8.3 g (64%) | 834.27 |
| 236 | 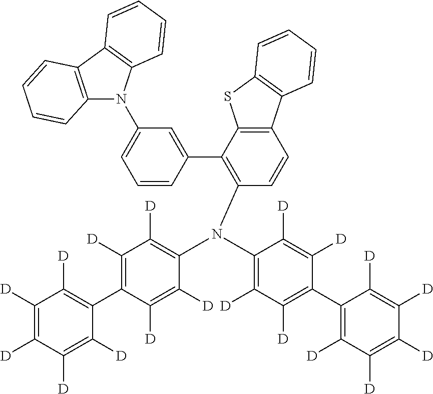 [SMILES: [2H]c1c([2H])c([2H])c(-c2c([2H])c([2H])c(N(c3ccc4c(sc5ccccc54)c3-c3cccc(-n4c5ccccc5c5ccccc54)c3)c3c([2H])c([2H])c(-c4c([2H])c([2H])c([2H])c([2H])c4[2H])c([2H])c3[2H])c([2H])c2[2H])c([2H])c1[2H]] | 7.9 g (67%) | 762.37 |
| 237 | 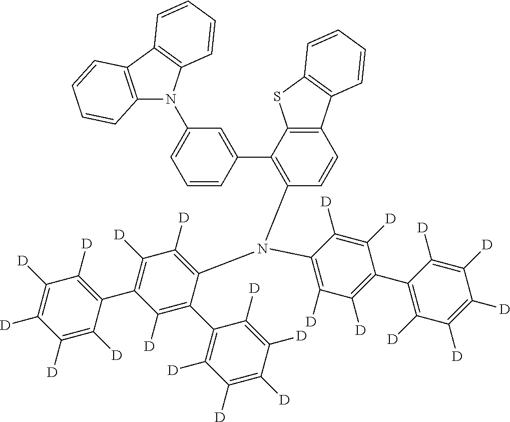 [SMILES: [2H]c1c([2H])c([2H])c(-c2c([2H])c([2H])c(N(c3ccc4c(sc5ccccc54)c3-c3cccc(-n4c5ccccc5c5ccccc54)c3)c3c([2H])c([2H])c(-c4c([2H])c([2H])c([2H])c([2H])c4[2H])c([2H])c3-c3c([2H])c([2H])c([2H])c([2H])c3[2H])c([2H])c2[2H])c([2H])c1[2H]] | 9.4 g (72%) | 842.43 |
| 238 | 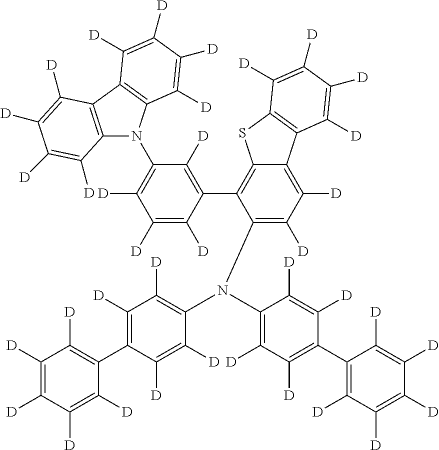 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4c(sc5c([2H])c([2H])c([2H])c([2H])c54)c3-c3c([2H])c([2H])c([2H])c(-n4c5c([2H])c([2H])c([2H])c([2H])c5c5c([2H])c([2H])c([2H])c([2H])c54)c3[2H])c([2H])c2[2H])c([2H])c1[2H]] | 8.1 g (67%) | 780.49 |
| 239 | 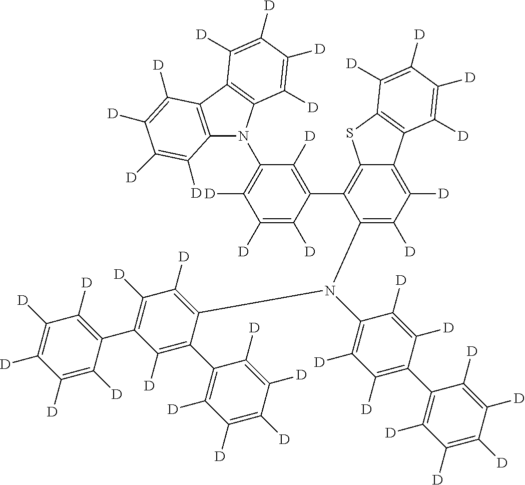 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3-c3c([2H])c([2H])c([2H])c([2H])c3[2H])c3c([2H])c([2H])c4c(sc5c([2H])c([2H])c([2H])c([2H])c54)c3-c3c([2H])c([2H])c([2H])c(-n4c5c([2H])c([2H])c([2H])c([2H])c5c5c([2H])c([2H])c([2H])c([2H])c54)c3[2H])c([2H])c2[2H])c([2H])c1[2H]] | 8.8 g (66%) | 860.54 |

TABLE 20

| | SUB 2-30 | |
|---|---|---|
| Compound | SUB 2-36 | SUB 1 |
| 240 | 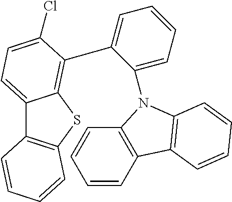 [SMILES: Clc1ccc2c(sc3ccccc32)c1-c1ccccc1-n1c2ccccc2c2ccccc21] | 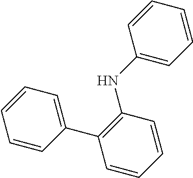 [SMILES: c1ccc(Nc2ccccc2-c2ccccc2)cc1] |
| 241 | | 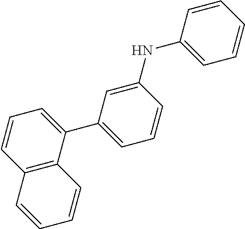 [SMILES: c1ccc(Nc2cccc(-c3cccc4ccccc34)c2)cc1] |
| 242 | | 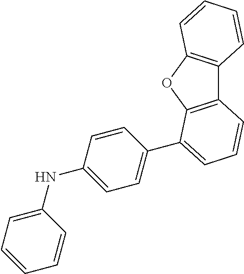 [SMILES: c1ccc(Nc2ccc(-c3cccc4c3oc3ccccc34)cc2)cc1] |
| 243 | | 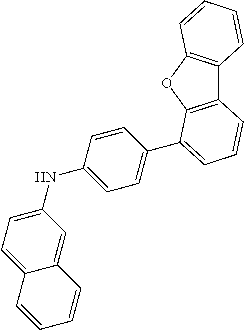 [SMILES: c1ccc2cc(Nc3ccc(-c4cccc5c4oc4ccccc45)cc3)ccc2c1] |
| 244 | | 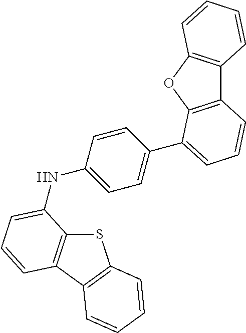 [SMILES: c1ccc2c(c1)oc1c(-c3ccc(Nc4cccc5c4sc4ccccc45)cc3)cccc12] |
| 245 | | 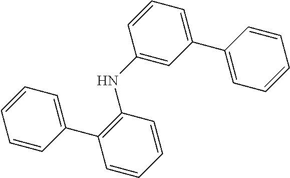 [SMILES: c1ccc(-c2cccc(Nc3ccccc3-c3ccccc3)c2)cc1] |
| 246 | | 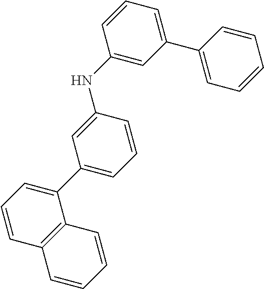 [SMILES: c1ccc(-c2cccc(Nc3cccc(-c4cccc5ccccc45)c3)c2)cc1] |
| 247 | | 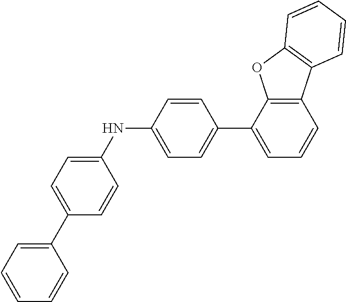 [SMILES: c1ccc(-c2ccc(Nc3ccc(-c4cccc5c4oc4ccccc45)cc3)cc2)cc1] |
| 248 | | 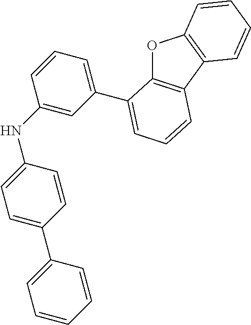 [SMILES: c1ccc(-c2ccc(Nc3cccc(-c4cccc5c4oc4ccccc45)c3)cc2)cc1] |
| 249 | | 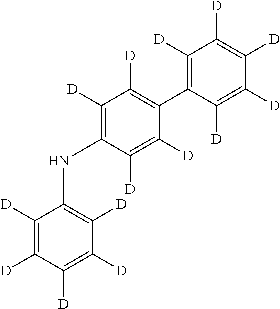 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] |
| 250 | | 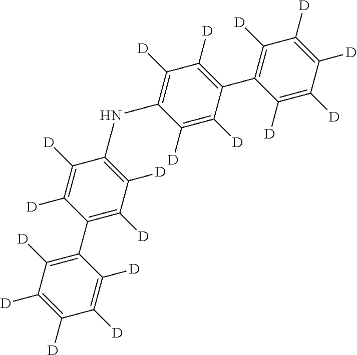 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] |
| 251 | 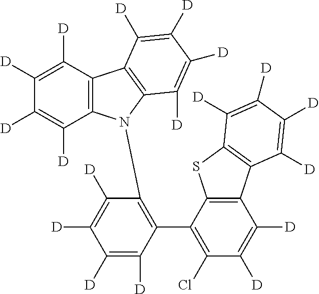 [SMILES: [2H]c1c([2H])c([2H])c(-n2c3c([2H])c([2H])c([2H])c([2H])c3c3c([2H])c([2H])c([2H])c([2H])c32)c(-c2c(Cl)c([2H])c([2H])c3c2sc2c([2H])c([2H])c([2H])c([2H])c23)c1[2H]] | 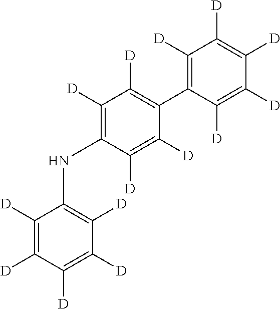 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] |
| 252 | | 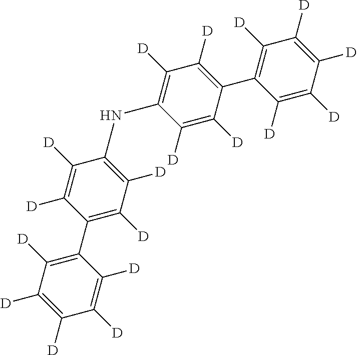 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] |

| | | Obtained | |
|---|---|---|---|
| Compound | Product | amount | [M + H]⁺ |
| 240 | 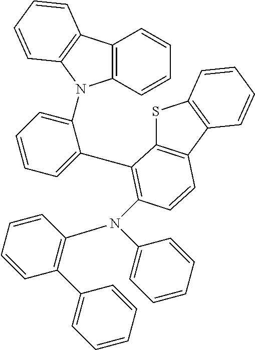 [SMILES: c1ccc(-c2ccccc2N(c2ccccc2)c2ccc3c(sc4ccccc43)c2-c2ccccc2-n2c3ccccc3c3ccccc32)cc1] | 7.5 g (72%) | 668.23 |
| 241 | 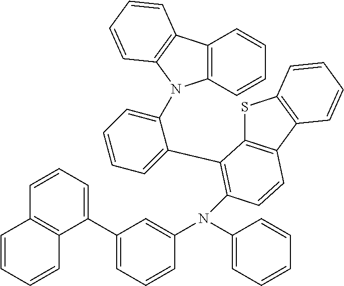 [SMILES: c1ccc(N(c2cccc(-c3cccc4ccccc34)c2)c2ccc3c(sc4ccccc43)c2-c2ccccc2-n2c3ccccc3c3ccccc32)cc1] | 7.6 g (68%) | 718.24 |
| 242 | 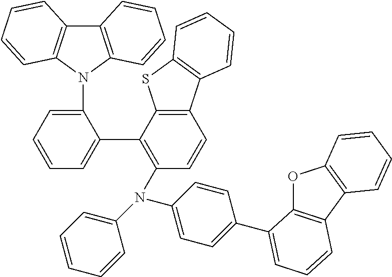 [SMILES: c1ccc(N(c2ccc(-c3cccc4c3oc3ccccc34)cc2)c2ccc3c(sc4ccccc43)c2-c2ccccc2-n2c3ccccc3c3ccccc32)cc1] | 8.6 g (73%) | 758.24 |
| 243 | 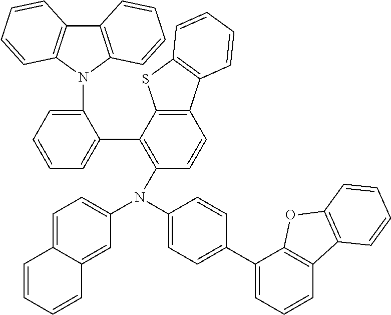 [SMILES: c1ccc(-n2c3ccccc3c3ccccc32)c(-c2c(N(c3ccc(-c4cccc5c4oc4ccccc45)cc3)c3ccc4ccccc4c3)ccc3c2sc2ccccc23)c1] | 9.1 g (72%) | 808.25 |
| 244 | 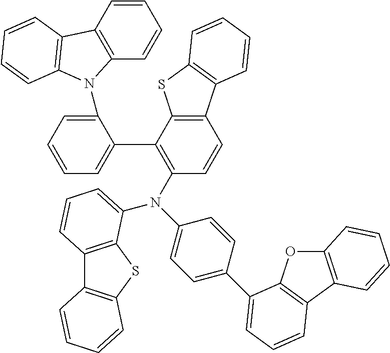 [SMILES: c1ccc(-n2c3ccccc3c3ccccc32)c(-c2c(N(c3ccc(-c4cccc5c4oc4ccccc45)cc3)c3cccc4c3sc3ccccc34)ccc3c2sc2ccccc23)c1] | 8.7 g (65%) | 864.23 |
| 245 | 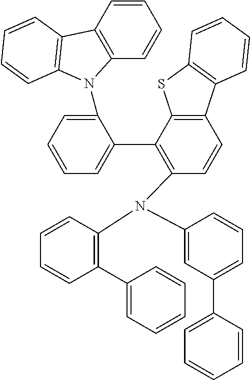 [SMILES: c1ccc(-c2cccc(N(c3ccccc3-c3ccccc3)c3ccc4c(sc5ccccc54)c3-c3ccccc3-n3c4ccccc4c4ccccc43)c2)cc1] | 7.1 g (61%) | 744.26 |
| 246 | 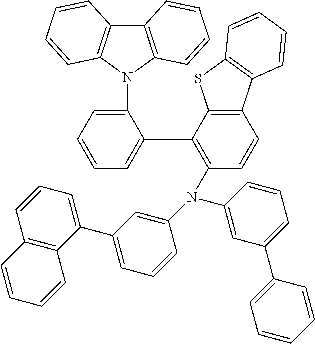 [SMILES: c1ccc(-c2cccc(N(c3cccc(-c4cccc5ccccc45)c3)c3ccc4c(sc5ccccc54)c3-c3ccccc3-n3c4ccccc4c4ccccc43)c2)cc1] | 8.4 g (68%) | 794.28 |
| 247 | 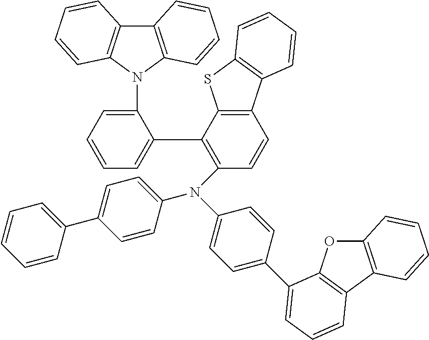 [SMILES: c1ccc(-c2ccc(N(c3ccc(-c4cccc5c4oc4ccccc45)cc3)c3ccc4c(sc5ccccc54)c3-c3ccccc3-n3c4ccccc4c4ccccc43)cc2)cc1] | 9.3 g (72%) | 834.27 |
| 248 | 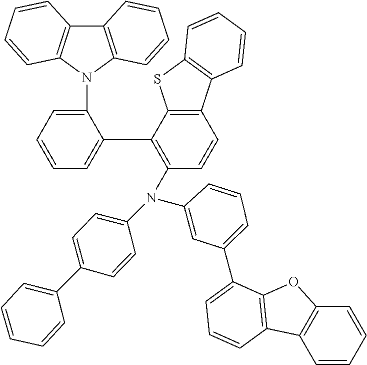 [SMILES: c1ccc(-c2ccc(N(c3cccc(-c4cccc5c4oc4ccccc45)c3)c3ccc4c(sc5ccccc54)c3-c3ccccc3-n3c4ccccc4c4ccccc43)cc2)cc1] | 8.8 g (68%) | 834.27 |
| 249 | 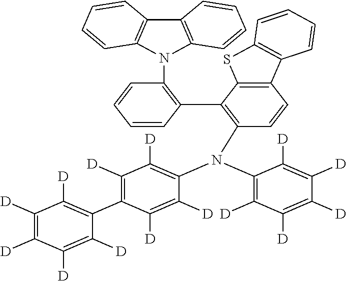 [SMILES: [2H]c1c([2H])c([2H])c(-c2c([2H])c([2H])c(N(c3ccc4c(sc5ccccc54)c3-c3ccccc3-n3c4ccccc4c4ccccc43)c3c([2H])c([2H])c([2H])c([2H])c3[2H])c([2H])c2[2H])c([2H])c1[2H]] | 7.8 g (73%) | 682.32 |
| 250 | 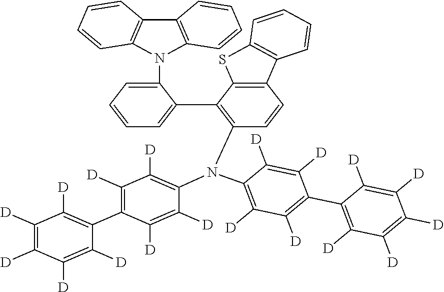 [SMILES: [2H]c1c([2H])c([2H])c(-c2c([2H])c([2H])c(N(c3ccc4c(sc5ccccc54)c3-c3ccccc3-n3c4ccccc4c4ccccc43)c3c([2H])c([2H])c(-c4c([2H])c([2H])c([2H])c([2H])c4[2H])c([2H])c3[2H])c([2H])c2[2H])c([2H])c1[2H]] | 8.3 g (70%) | 762.37 |
| 251 | 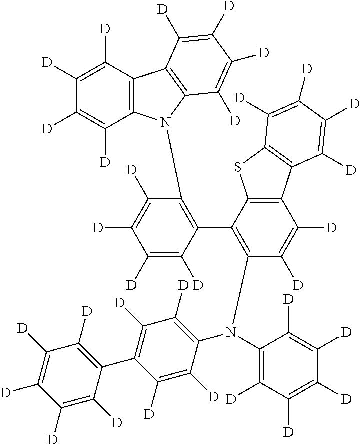 [SMILES: [2H]c1c([2H])c([2H])c(-c2c([2H])c([2H])c(N(c3c([2H])c([2H])c([2H])c([2H])c3[2H])c3c([2H])c([2H])c4c(sc5c([2H])c([2H])c([2H])c([2H])c54)c3-c3c([2H])c([2H])c([2H])c([2H])c3-n3c4c([2H])c([2H])c([2H])c([2H])c4c4c([2H])c([2H])c([2H])c([2H])c43)c([2H])c2[2H])c([2H])c1[2H]] | 7.1 g (65%) | 700.43 |
| 252 | 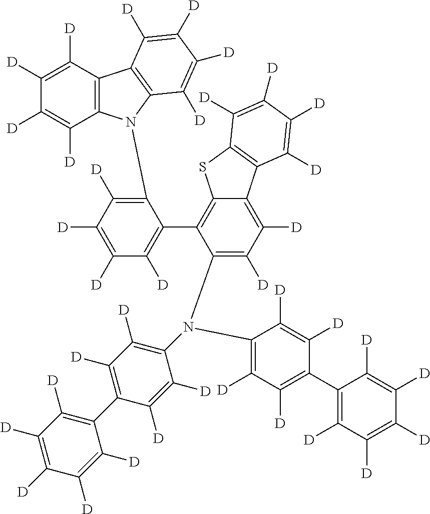 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4c(sc5c([2H])c([2H])c([2H])c([2H])c54)c3-c3c([2H])c([2H])c([2H])c([2H])c3-n3c4c([2H])c([2H])c([2H])c([2H])c4c4c([2H])c([2H])c([2H])c([2H])c43)c([2H])c2[2H])c([2H])c1[2H]] | 7.5 g (62%) | 780.49 |

### Example 1: Manufacturing of Organic Electroluminescent Device (Green

A positive electrode was formed with ITO on a substrate on which a reflective layer was formed and surface-treated with N₂ plasma or UV-ozone. HAT-CN was deposited above the positive electrode in a thickness of 10 nm as a hole injection layer (HIL). Subsequently, a hole transport layer (HTL) was formed by depositing N4,N4,N4′,N4′-tetra([1,1′-biphenyl]-4-yl)-[1,1′-biphenyl]-4,4′-diamine in a thickness of 110 nm.

A hole transport auxiliary layer was formed by forming Compound 1 of the present invention above the hole transport layer by vacuum deposition in a thickness of 40 nm, and Ir(ppy)₃[tris(2-phenylpyridine)-iridium] as a dopant was doped at about 5% while 4,4′-N,N′-dicarbazole-biphenyl(CBP) as an emitting layer (EML) was deposited above the hole transport auxiliary layer in a thickness of 35 nm.

An electron transport layer (ETL) was deposited above the light emitting layer (EML) in a thickness of 30 nm by mixing anthracene derivative and Liq at 1:1, and Liq as an electron injection layer (EIL) was deposited above the electron transport layer (ETL) in a thickness of 1 nm. Then, a mixture mixing magnesium and silver (Ag) at 1:4 was deposited in a thickness of 16 nm as a negative electrode, and N4,N4′-bis[4-[bis(3-methylphenyl)amino]phenyl]-N4,N4′-diphenyl-[1,1′-biphenyl]-4,4′-diamine (DNTPD) as a capping layer was deposited above the negative electrode in a thickness of 60 nm. An organic electroluminescent device was manufactured by bonding a sealcap containing a moisture absorbent to the capping layer using a UV curable adhesive to protect the organic electroluminescent device from O₂ or moisture in the atmosphere.

### Examples 2 to 252

An organic electroluminescent device was manufactured in the same manner as Example 1 except that compounds listed in Table 21 below were used instead of Compound 1 as the hole transport auxiliary layer in Example 1.

### Comparative Examples 1 to 3

An organic electroluminescent device was manufactured in the same manner as Example 1 except that Compounds A to C were used instead of Compound 1 as the hole transport auxiliary layer in Example 1.

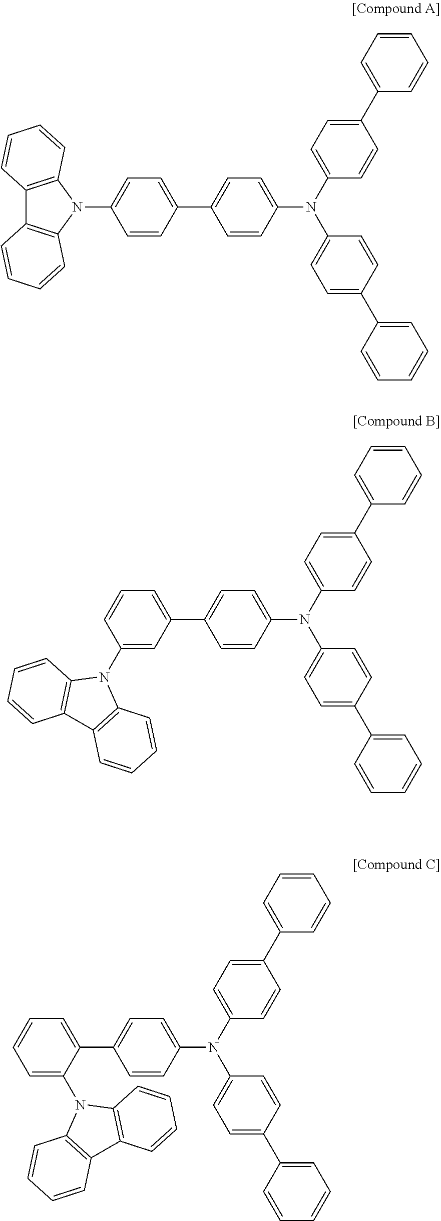 [SMILES: c1ccc(-c2ccc(N(c3ccc(-c4ccccc4)cc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4cccc(-n5c6ccccc6c6ccccc65)c4)cc3)cc2)cc1 ; c1ccc(-c2ccc(N(c3ccc(-c4ccccc4)cc3)c3ccc(-c4ccccc4-n4c5ccccc5c5ccccc54)cc3)cc2)cc1]

### Experimental Example 1: Device Performance Analysis

For the organic electroluminescent devices manufactured in Examples 1 to 252 and Comparative Examples 1 to 3, electroluminescent characteristics when driven at a current of 10 mA/cm² and lifetime reduced by 95% when driven at a constant current of 20 mA/cm² were measured, and a result of measurement is expressed in Table 21.

TABLE 21

| | Hole transport | Driving | | | | | |
|---|---|---|---|---|---|---|---|
| | auxiliary | voltage | Efficiency | | Lifetime | Color | |
| Items | layer | (V) | Cd/A | EQE(%) | T95(hrs) | CIEx | CIEy |
| Example 1 | Compound 1 | 3.86 | 144.2 | 33.7 | 260 | 0.272 | 0.697 |
| Example 2 | Compound 2 | 3.87 | 126.9 | 29.6 | 240 | 0.275 | 0.695 |
| Example 3 | Compound 3 | 3.77 | 131.2 | 30.6 | 220 | 0.274 | 0.696 |
| Example 4 | Compound 4 | 3.83 | 120.0 | 28.1 | 245 | 0.268 | 0.700 |
| Example 5 | Compound 5 | 3.78 | 134.0 | 31.8 | 210 | 0.256 | 0.707 |
| Example 6 | Compound 6 | 3.84 | 126.9 | 29.7 | 230 | 0.258 | 0.708 |
| Example 7 | Compound 7 | 3.84 | 146.2 | 34.4 | 230 | 0.258 | 0.708 |
| Example 8 | Compound 8 | 3.53 | 144.9 | 34.3 | 220 | 0.237 | 0.723 |
| Example 9 | Compound 9 | 3.55 | 147.1 | 34.8 | 240 | 0.250 | 0.713 |
| Example 10 | Compound 10 | 3.53 | 147.1 | 34.8 | 255 | 0.250 | 0.713 |
| Example 11 | Compound 11 | 3.62 | 141.3 | 33.3 | 285 | 0.259 | 0.707 |
| Example 12 | Compound 12 | 3.61 | 145.2 | 34.3 | 280 | 0.256 | 0.709 |
| Example 13 | Compound 13 | 3.65 | 143.9 | 33.8 | 275 | 0.258 | 0.708 |
| Example 14 | Compound 14 | 3.61 | 147.5 | 34.7 | 306 | 0.254 | 0.711 |
| Example 15 | Compound 15 | 3.70 | 130.1 | 31.4 | 327 | 0.236 | 0.719 |
| Example 16 | Compound 16 | 3.64 | 143.7 | 33.2 | 250 | 0.277 | 0.702 |
| Example 17 | Compound 17 | 3.61 | 126.4 | 29.1 | 230 | 0.280 | 0.700 |
| Example 18 | Compound 18 | 3.68 | 130.7 | 30.1 | 210 | 0.279 | 0.701 |
| Example 19 | Compound 19 | 3.74 | 119.5 | 27.6 | 235 | 0.273 | 0.705 |
| Example 20 | Compound 20 | 3.57 | 133.5 | 31.3 | 200 | 0.261 | 0.712 |
| Example 21 | Compound 21 | 3.58 | 126.4 | 29.2 | 220 | 0.263 | 0.713 |
| Example 22 | Compound 22 | 3.68 | 145.7 | 33.9 | 220 | 0.263 | 0.713 |
| Example 23 | Compound 23 | 3.78 | 144.4 | 33.8 | 210 | 0.242 | 0.728 |
| Example 24 | Compound 24 | 3.64 | 146.6 | 34.3 | 230 | 0.255 | 0.718 |
| Example 25 | Compound 25 | 3.55 | 146.6 | 34.3 | 245 | 0.255 | 0.718 |
| Example 26 | Compound 26 | 3.61 | 140.8 | 32.8 | 275 | 0.264 | 0.712 |
| Example 27 | Compound 27 | 3.64 | 144.7 | 33.8 | 270 | 0.261 | 0.714 |
| Example 28 | Compound 28 | 3.59 | 143.4 | 33.3 | 265 | 0.263 | 0.713 |
| Example 29 | Compound 29 | 3.62 | 147.0 | 34.2 | 296 | 0.259 | 0.716 |
| Example 30 | Compound 30 | 3.66 | 129.6 | 30.9 | 317 | 0.241 | 0.724 |
| Example 31 | Compound 31 | 3.65 | 157.6 | 36.3 | 235 | 0.265 | 0.698 |
| Example 32 | Compound 32 | 3.68 | 152.3 | 35.0 | 215 | 0.269 | 0.696 |
| Example 33 | Compound 33 | 3.54 | 158.6 | 36.8 | 236 | 0.244 | 0.715 |
| Example 34 | Compound 34 | 3.64 | 154.6 | 35.8 | 232 | 0.249 | 0.712 |
| Example 35 | Compound 35 | 3.65 | 158.1 | 36.7 | 250 | 0.244 | 0.715 |
| Example 36 | Compound 36 | 3.55 | 153.2 | 35.4 | 253 | 0.362 | 0.701 |
| Example 37 | Compound 37 | 3.58 | 160.1 | 37.1 | 240 | 0.255 | 0.706 |
| Example 38 | Compound 38 | 3.66 | 134.5 | 32.3 | 245 | 0.251 | 0.702 |
| Example 39 | Compound 39 | 3.67 | 137.7 | 32.6 | 253 | 0.252 | 0.704 |
| Example 40 | Compound 40 | 3.63 | 126.2 | 33.0 | 260 | 0.211 | 0.731 |
| Example 41 | Compound 41 | 3.69 | 151.1 | 35.8 | 273 | 0.257 | 0.701 |
| Example 42 | Compound 42 | 3.59 | 141.9 | 33.3 | 282 | 0.264 | 0.696 |
| Example 43 | Compound 43 | 3.69 | 137.3 | 32.1 | 300 | 0.262 | 0.698 |
| Example 44 | Compound 44 | 3.76 | 157.1 | 35.8 | 220 | 0.270 | 0.703 |
| Example 45 | Compound 45 | 3.71 | 151.8 | 34.5 | 200 | 0.274 | 0.701 |
| Example 46 | Compound 46 | 3.70 | 158.1 | 36.3 | 221 | 0.249 | 0.720 |
| Example 47 | Compound 47 | 3.75 | 154.1 | 35.3 | 217 | 0.254 | 0.717 |
| Example 48 | Compound 48 | 3.78 | 157.6 | 36.2 | 235 | 0.249 | 0.720 |
| Example 49 | Compound 49 | 3.79 | 152.7 | 34.9 | 238 | 0.367 | 0.706 |
| Example 50 | Compound 50 | 3.82 | 159.6 | 36.6 | 225 | 0.260 | 0.711 |
| Example 51 | Compound 51 | 3.68 | 134.0 | 31.8 | 230 | 0.256 | 0.707 |
| Example 52 | Compound 52 | 3.61 | 137.2 | 32.1 | 238 | 0.257 | 0.709 |
| Example 53 | Compound 53 | 3.60 | 125.7 | 32.5 | 245 | 0.216 | 0.736 |
| Example 54 | Compound 54 | 3.52 | 150.6 | 35.3 | 258 | 0.262 | 0.706 |
| Example 55 | Compound 55 | 3.50 | 141.4 | 32.8 | 267 | 0.269 | 0.701 |
| Example 56 | Compound 56 | 3.73 | 136.8 | 31.6 | 285 | 0.267 | 0.703 |
| Example 57 | Compound 57 | 3.73 | 157.6 | 36.3 | 215 | 0.273 | 0.706 |
| Example 58 | Compound 58 | 3.69 | 152.3 | 35.0 | 195 | 0.277 | 0.704 |
| Example 59 | Compound 59 | 3.77 | 158.6 | 36.8 | 216 | 0.252 | 0.723 |
| Example 60 | Compound 60 | 3.67 | 154.6 | 35.8 | 212 | 0.257 | 0.720 |
| Example 61 | Compound 61 | 3.78 | 158.1 | 36.7 | 230 | 0.252 | 0.723 |
| Example 62 | Compound 62 | 3.62 | 153.2 | 35.4 | 233 | 0.370 | 0.709 |
| Example 63 | Compound 63 | 3.60 | 160.1 | 37.1 | 220 | 0.263 | 0.714 |
| Example 64 | Compound 64 | 3.59 | 134.5 | 32.3 | 225 | 0.259 | 0.710 |
| Example 65 | Compound 65 | 3.69 | 137.7 | 32.6 | 233 | 0.260 | 0.712 |
| Example 66 | Compound 66 | 3.56 | 126.2 | 33.0 | 240 | 0.219 | 0.739 |
| Example 67 | Compound 67 | 3.58 | 151.1 | 35.8 | 253 | 0.265 | 0.709 |
| Example 68 | Compound 68 | 3.66 | 141.9 | 33.3 | 262 | 0.272 | 0.704 |
| Example 69 | Compound 69 | 3.56 | 137.3 | 32.1 | 280 | 0.270 | 0.706 |
| Example 70 | Compound 70 | 3.59 | 158.1 | 36.8 | 210 | 0.276 | 0.709 |
| Example 71 | Compound 71 | 3.61 | 152.8 | 35.5 | 190 | 0.280 | 0.707 |
| Example 72 | Compound 72 | 3.68 | 143.9 | 33.7 | 219 | 0.268 | 0.700 |
| Example 73 | Compound 73 | 3.71 | 137.7 | 32.3 | 228 | 0.778 | 0.692 |
| Example 74 | Compound 74 | 3.73 | 139.8 | 32.6 | 235 | 0.274 | 0.696 |
| Example 75 | Compound 75 | 3.75 | 136.2 | 32.6 | 228 | 0.239 | 0.719 |
| Example 76 | Compound 76 | 3.79 | 139.5 | 33.4 | 231 | 0.251 | 0.709 |
| Example 77 | Compound 77 | 3.76 | 135.7 | 32.6 | 240 | 0.228 | 0.727 |
| Example 78 | Compound 78 | 3.73 | 133.3 | 32.1 | 245 | 0.226 | 0.729 |
| Example 79 | Compound 79 | 3.77 | 131.3 | 31.5 | 233 | 0.238 | 0.720 |
| Example 80 | Compound 80 | 3.84 | 139.0 | 33.2 | 227 | 0.245 | 0.714 |
| Example 81 | Compound 81 | 3.70 | 135.7 | 32.6 | 267 | 0.228 | 0.727 |
| Example 82 | Compound 82 | 3.69 | 133.3 | 32.1 | 265 | 0.226 | 0.729 |
| Example 83 | Compound 83 | 3.71 | 137.2 | 32.5 | 270 | 0.245 | 0.717 |
| Example 84 | Compound 84 | 3.70 | 136.7 | 32.5 | 305 | 0.243 | 0.717 |
| Example 85 | Compound 85 | 3.75 | 139.2 | 33.0 | 310 | 0.244 | 0.717 |
| Example 86 | Compound 86 | 3.78 | 144.3 | 33.7 | 326 | 0.264 | 0.704 |
| Example 87 | Compound 87 | 3.63 | 126.6 | 30.1 | 248 | 0.209 | 0.725 |
| Example 88 | Compound 88 | 3.66 | 128.0 | 30.4 | 263 | 0.214 | 0.721 |
| Example 89 | Compound 89 | 3.65 | 129.2 | 29.5 | 281 | 0.211 | 0.723 |
| Example 90 | Compound 90 | 3.64 | 127.2 | 30.6 | 287 | 0.232 | 0.713 |
| Example 91 | Compound 91 | 3.65 | 144.6 | 34.5 | 250 | 0.238 | 0.709 |
| Example 92 | Compound 92 | 3.67 | 126.6 | 29.3 | 240 | 0.229 | 0.715 |
| Example 93 | Compound 93 | 3.68 | 126.3 | 30.8 | 232 | 0.217 | 0.721 |
| Example 94 | Compound 94 | 3.64 | 129.8 | 27.6 | 250 | 0.200 | 0.727 |
| Example 95 | Compound 95 | 3.66 | 123.9 | 26.3 | 232 | 0.197 | 0.728 |
| Example 96 | Compound 96 | 3.69 | 145.6 | 34.0 | 258 | 0.261 | 0.695 |
| Example 97 | Compound 97 | 3.64 | 148.6 | 34.7 | 267 | 0.259 | 0.696 |
| Example 98 | Compound 98 | 3.63 | 141.1 | 33.5 | 302 | 0.515 | 0.707 |
| Example 99 | Compound 99 | 3.69 | 144.5 | 33.9 | 314 | 0.259 | 0.695 |
| Example 100 | Compound 100 | 3.69 | 126.3 | 29.8 | 233 | 0.219 | 0.730 |
| Example 101 | Compound 101 | 3.72 | 127.7 | 30.1 | 248 | 0.224 | 0.726 |
| Example 102 | Compound 102 | 3.72 | 128.9 | 29.2 | 266 | 0.221 | 0.728 |
| Example 103 | Compound 103 | 3.77 | 126.9 | 30.3 | 272 | 0.242 | 0.718 |
| Example 104 | Compound 104 | 3.74 | 144.3 | 34.2 | 235 | 0.248 | 0.714 |
| Example 105 | Compound 105 | 3.66 | 126.3 | 29.0 | 225 | 0.239 | 0.720 |
| Example 106 | Compound 106 | 3.69 | 126.0 | 30.5 | 217 | 0.227 | 0.726 |
| Example 107 | Compound 107 | 3.65 | 129.5 | 27.3 | 235 | 0.210 | 0.732 |
| Example 108 | Compound 108 | 3.63 | 123.6 | 26.0 | 217 | 0.207 | 0.733 |
| Example 109 | Compound 109 | 3.76 | 145.3 | 33.7 | 243 | 0.271 | 0.700 |
| Example 110 | Compound 110 | 3.75 | 148.3 | 34.4 | 252 | 0.269 | 0.701 |
| Example 111 | Compound 111 | 3.80 | 140.8 | 33.2 | 287 | 0.525 | 0.712 |
| Example 112 | Compound 112 | 3.78 | 144.2 | 33.6 | 299 | 0.269 | 0.700 |
| Example 113 | Compound 113 | 3.65 | 127.0 | 29.6 | 233 | 0.220 | 0.725 |
| Example 114 | Compound 114 | 3.70 | 125.9 | 29.9 | 248 | 0.225 | 0.721 |
| Example 115 | Compound 115 | 3.69 | 134.6 | 29.0 | 266 | 0.222 | 0.723 |
| Example 116 | Compound 116 | 3.75 | 121.7 | 30.1 | 272 | 0.243 | 0.713 |
| Example 117 | Compound 117 | 3.70 | 137.6 | 34.0 | 235 | 0.249 | 0.709 |
| Example 118 | Compound 118 | 3.64 | 131.6 | 28.8 | 225 | 0.240 | 0.715 |
| Example 119 | Compound 119 | 3.77 | 133.4 | 30.3 | 217 | 0.228 | 0.721 |
| Example 120 | Compound 120 | 3.66 | 136.2 | 27.1 | 235 | 0.211 | 0.727 |
| Example 121 | Compound 121 | 3.71 | 140.0 | 25.8 | 217 | 0.208 | 0.728 |
| Example 122 | Compound 122 | 3.65 | 143.6 | 33.5 | 243 | 0.272 | 0.695 |
| Example 123 | Compound 123 | 3.65 | 137.0 | 34.2 | 252 | 0.270 | 0.696 |
| Example 124 | Compound 124 | 3.75 | 136.8 | 33.0 | 287 | 0.526 | 0.707 |
| Example 125 | Compound 125 | 3.58 | 142.4 | 33.4 | 299 | 0.270 | 0.695 |
| Example 126 | Compound 126 | 3.61 | 136.7 | 29.4 | 233 | 0.221 | 0.720 |
| Example 127 | Compound 127 | 3.61 | 135.7 | 29.7 | 248 | 0.226 | 0.716 |
| Example 128 | Compound 128 | 3.76 | 126.5 | 30.8 | 230 | 0.222 | 0.729 |
| Example 129 | Compound 129 | 3.77 | 125.4 | 30.2 | 211 | 0.224 | 0.728 |
| Example 130 | Compound 130 | 3.81 | 134.1 | 32.1 | 205 | 0.238 | 0.720 |
| Example 131 | Compound 131 | 3.82 | 121.2 | 29.5 | 222 | 0.238 | 0.715 |
| Example 132 | Compound 132 | 3.82 | 137.1 | 33.1 | 236 | 0.229 | 0.725 |
| Example 133 | Compound 133 | 3.80 | 131.1 | 31.7 | 243 | 0.224 | 0.729 |
| Example 134 | Compound 134 | 3.83 | 132.9 | 32.3 | 240 | 0.233 | 0.721 |
| Example 135 | Compound 135 | 3.77 | 135.7 | 32.7 | 235 | 0.245 | 0.713 |
| Example 136 | Compound 136 | 3.73 | 139.5 | 33.4 | 270 | 0.251 | 0.709 |
| Example 137 | Compound 137 | 3.68 | 143.1 | 33.5 | 271 | 0.265 | 0.702 |
| Example 138 | Compound 138 | 3.56 | 136.5 | 32.2 | 275 | 0.252 | 0.712 |
| Example 139 | Compound 139 | 3.74 | 136.3 | 32.1 | 242 | 0.263 | 0.703 |
| Example 140 | Compound 140 | 3.78 | 141.9 | 33.5 | 305 | 0.253 | 0.711 |
| Example 141 | Compound 141 | 3.73 | 136.2 | 32.6 | 310 | 0.239 | 0.719 |
| Example 142 | Compound 142 | 3.77 | 135.2 | 32.7 | 300 | 0.244 | 0.712 |
| Example 143 | Compound 143 | 3.74 | 131.0 | 31.8 | 235 | 0.257 | 0.705 |
| Example 144 | Compound 144 | 3.78 | 126.9 | 30.8 | 230 | 0.225 | 0.728 |
| Example 145 | Compound 145 | 3.81 | 126.8 | 30.7 | 220 | 0.226 | 0.728 |
| Example 146 | Compound 146 | 3.83 | 128.6 | 31.1 | 220 | 0.226 | 0.728 |
| Example 147 | Compound 147 | 3.80 | 139.0 | 33.2 | 285 | 0.245 | 0.714 |
| Example 148 | Compound 148 | 3.82 | 143.3 | 34.1 | 232 | 0.249 | 0.712 |
| Example 149 | Compound 149 | 3.79 | 139.5 | 33.4 | 270 | 0.251 | 0.709 |
| Example 150 | Compound 150 | 3.75 | 131.0 | 31.8 | 245 | 0.257 | 0.705 |
| Example 151 | Compound 151 | 3.74 | 136.5 | 32.2 | 225 | 0.252 | 0.712 |
| Example 152 | Compound 152 | 3.72 | 137.2 | 32.5 | 270 | 0.271 | 0.696 |
| Example 153 | Compound 153 | 3.70 | 135.5 | 32.3 | 280 | 0.259 | 0.703 |
| Example 154 | Compound 154 | 3.70 | 134.7 | 32.2 | 313 | 0.262 | 0.701 |
| Example 155 | Compound 155 | 3.69 | 128.9 | 31.2 | 321 | 0.242 | 0.713 |
| Example 156 | Compound 156 | 3.72 | 130.6 | 31.4 | 230 | 0.259 | 0.707 |
| Example 157 | Compound 157 | 3.74 | 126.5 | 30.4 | 225 | 0.227 | 0.730 |
| Example 158 | Compound 158 | 3.79 | 126.4 | 30.3 | 215 | 0.228 | 0.730 |
| Example 159 | Compound 159 | 3.77 | 128.2 | 30.7 | 215 | 0.228 | 0.730 |
| Example 160 | Compound 160 | 3.79 | 138.6 | 32.8 | 280 | 0.247 | 0.716 |
| Example 161 | Compound 161 | 3.78 | 142.9 | 33.7 | 227 | 0.251 | 0.714 |
| Example 162 | Compound 162 | 3.79 | 139.1 | 33.0 | 265 | 0.253 | 0.711 |
| Example 163 | Compound 163 | 3.77 | 130.6 | 31.4 | 240 | 0.259 | 0.707 |
| Example 164 | Compound 164 | 3.75 | 136.1 | 31.8 | 220 | 0.254 | 0.714 |
| Example 165 | Compound 165 | 3.70 | 136.8 | 32.1 | 265 | 0.273 | 0.698 |
| Example 166 | Compound 166 | 3.71 | 135.1 | 31.9 | 275 | 0.261 | 0.705 |
| Example 167 | Compound 167 | 3.69 | 134.3 | 31.8 | 308 | 0.264 | 0.703 |
| Example 168 | Compound 168 | 3.70 | 128.5 | 30.8 | 316 | 0.244 | 0.715 |
| Example 169 | Compound 169 | 3.93 | 133.7 | 31.7 | 245 | 0.277 | 0.702 |
| Example 170 | Compound 170 | 3.94 | 116.4 | 27.6 | 225 | 0.280 | 0.700 |
| Example 171 | Compound 171 | 3.84 | 120.7 | 28.6 | 205 | 0.279 | 0.701 |
| Example 172 | Compound 172 | 3.90 | 109.5 | 26.1 | 230 | 0.273 | 0.705 |
| Example 173 | Compound 173 | 3.85 | 123.5 | 29.8 | 195 | 0.261 | 0.712 |
| Example 174 | Compound 174 | 3.91 | 116.4 | 27.7 | 215 | 0.263 | 0.713 |
| Example 175 | Compound 175 | 3.91 | 135.7 | 32.4 | 215 | 0.263 | 0.713 |
| Example 176 | Compound 176 | 3.60 | 134.4 | 32.3 | 205 | 0.242 | 0.728 |
| Example 177 | Compound 177 | 3.62 | 136.6 | 32.8 | 225 | 0.255 | 0.718 |
| Example 178 | Compound 178 | 3.60 | 136.6 | 32.8 | 240 | 0.255 | 0.718 |
| Example 179 | Compound 179 | 3.69 | 130.8 | 31.3 | 270 | 0.264 | 0.712 |
| Example 180 | Compound 180 | 3.68 | 134.7 | 32.3 | 265 | 0.261 | 0.714 |
| Example 181 | Compound 181 | 3.72 | 133.4 | 31.8 | 260 | 0.263 | 0.713 |
| Example 182 | Compound 182 | 3.68 | 137.0 | 32.7 | 291 | 0.259 | 0.716 |
| Example 183 | Compound 183 | 3.77 | 119.6 | 29.4 | 312 | 0.241 | 0.724 |
| Example 184 | Compound 184 | 3.71 | 133.2 | 31.2 | 235 | 0.282 | 0.707 |
| Example 185 | Compound 185 | 3.68 | 115.9 | 27.1 | 215 | 0.285 | 0.705 |
| Example 186 | Compound 186 | 3.75 | 120.2 | 28.1 | 195 | 0.284 | 0.706 |
| Example 187 | Compound 187 | 3.81 | 109.0 | 25.6 | 220 | 0.278 | 0.710 |
| Example 188 | Compound 188 | 3.64 | 123.0 | 29.3 | 185 | 0.266 | 0.717 |
| Example 189 | Compound 189 | 3.65 | 115.9 | 27.2 | 205 | 0.268 | 0.718 |
| Example 190 | Compound 190 | 3.75 | 135.2 | 31.9 | 205 | 0.268 | 0.718 |
| Example 191 | Compound 191 | 3.85 | 133.9 | 31.8 | 195 | 0.247 | 0.733 |
| Example 192 | Compound 192 | 3.71 | 136.1 | 32.3 | 215 | 0.260 | 0.723 |
| Example 193 | Compound 193 | 3.62 | 136.1 | 32.3 | 230 | 0.260 | 0.723 |
| Example 194 | Compound 194 | 3.68 | 130.3 | 30.8 | 260 | 0.269 | 0.717 |
| Example 195 | Compound 195 | 3.71 | 134.2 | 31.8 | 255 | 0.266 | 0.719 |
| Example 196 | Compound 196 | 3.66 | 132.9 | 31.3 | 250 | 0.268 | 0.718 |
| Example 197 | Compound 197 | 3.69 | 136.5 | 32.2 | 281 | 0.264 | 0.721 |
| Example 198 | Compound 198 | 3.73 | 119.1 | 28.9 | 302 | 0.246 | 0.729 |
| Example 199 | Compound 199 | 3.72 | 147.1 | 34.3 | 220 | 0.270 | 0.703 |
| Example 200 | Compound 200 | 3.75 | 141.8 | 33.0 | 200 | 0.274 | 0.701 |
| Example 201 | Compound 201 | 3.61 | 148.1 | 34.8 | 221 | 0.249 | 0.720 |
| Example 202 | Compound 202 | 3.71 | 144.1 | 33.8 | 217 | 0.254 | 0.717 |
| Example 203 | Compound 203 | 3.72 | 147.6 | 34.7 | 235 | 0.249 | 0.720 |
| Example 204 | Compound 204 | 3.62 | 142.7 | 33.4 | 238 | 0.367 | 0.706 |
| Example 205 | Compound 205 | 3.65 | 149.6 | 35.1 | 225 | 0.260 | 0.711 |
| Example 206 | Compound 206 | 3.73 | 124.0 | 30.3 | 230 | 0.256 | 0.707 |
| Example 207 | Compound 207 | 3.74 | 127.2 | 30.6 | 238 | 0.257 | 0.709 |
| Example 208 | Compound 208 | 3.70 | 115.7 | 31.0 | 245 | 0.216 | 0.736 |
| Example 209 | Compound 209 | 3.76 | 140.6 | 33.8 | 258 | 0.262 | 0.706 |
| Example 210 | Compound 210 | 3.66 | 131.4 | 31.3 | 267 | 0.269 | 0.701 |
| Example 211 | Compound 211 | 3.76 | 126.8 | 30.1 | 285 | 0.267 | 0.703 |
| Example 212 | Compound 212 | 3.83 | 146.6 | 33.8 | 205 | 0.275 | 0.708 |
| Example 213 | Compound 213 | 3.78 | 141.3 | 32.5 | 185 | 0.279 | 0.706 |
| Example 214 | Compound 214 | 3.77 | 147.6 | 34.3 | 206 | 0.254 | 0.725 |
| Example 215 | Compound 215 | 3.82 | 143.6 | 33.3 | 202 | 0.259 | 0.722 |
| Example 216 | Compound 216 | 3.85 | 147.1 | 34.2 | 220 | 0.254 | 0.725 |
| Example 217 | Compound 217 | 3.86 | 142.2 | 32.9 | 223 | 0.372 | 0.711 |
| Example 218 | Compound 218 | 3.89 | 149.1 | 34.6 | 210 | 0.265 | 0.716 |
| Example 219 | Compound 219 | 3.75 | 123.5 | 29.8 | 215 | 0.261 | 0.712 |
| Example 220 | Compound 220 | 3.68 | 126.7 | 30.1 | 223 | 0.262 | 0.714 |
| Example 221 | Compound 221 | 3.67 | 115.2 | 30.5 | 205 | 0.221 | 0.741 |
| Example 222 | Compound 222 | 3.59 | 140.1 | 33.3 | 198 | 0.267 | 0.711 |
| Example 223 | Compound 223 | 3.57 | 130.9 | 30.8 | 252 | 0.274 | 0.706 |
| Example 224 | Compound 224 | 3.80 | 126.3 | 29.6 | 270 | 0.272 | 0.708 |
| Example 225 | Compound 225 | 3.80 | 147.1 | 34.3 | 200 | 0.278 | 0.711 |
| Example 226 | Compound 226 | 3.76 | 141.8 | 33.0 | 195 | 0.282 | 0.709 |
| Example 227 | Compound 227 | 3.84 | 148.1 | 34.8 | 201 | 0.257 | 0.728 |
| Example 228 | Compound 228 | 3.74 | 144.1 | 33.8 | 197 | 0.262 | 0.725 |
| Example 229 | Compound 229 | 3.85 | 147.6 | 34.7 | 215 | 0.257 | 0.728 |
| Example 230 | Compound 230 | 3.69 | 142.7 | 33.4 | 218 | 0.375 | 0.714 |
| Example 231 | Compound 231 | 3.67 | 149.6 | 35.1 | 205 | 0.268 | 0.719 |
| Example 232 | Compound 232 | 3.66 | 124.0 | 30.3 | 210 | 0.264 | 0.715 |
| Example 233 | Compound 233 | 3.76 | 127.2 | 30.6 | 218 | 0.265 | 0.717 |
| Example 234 | Compound 234 | 3.63 | 115.7 | 31.0 | 213 | 0.224 | 0.744 |
| Example 235 | Compound 235 | 3.65 | 140.6 | 33.8 | 225 | 0.270 | 0.714 |
| Example 236 | Compound 236 | 3.73 | 131.4 | 31.3 | 247 | 0.277 | 0.709 |
| Example 237 | Compound 237 | 3.63 | 126.8 | 30.1 | 265 | 0.275 | 0.711 |
| Example 238 | Compound 238 | 3.66 | 147.6 | 34.8 | 195 | 0.281 | 0.714 |
| Example 239 | Compound 239 | 3.68 | 142.3 | 33.5 | 175 | 0.285 | 0.712 |
| Example 240 | Compound 240 | 3.75 | 133.4 | 31.7 | 204 | 0.273 | 0.705 |
| Example 241 | Compound 241 | 3.78 | 127.2 | 30.3 | 213 | 0.783 | 0.697 |
| Example 242 | Compound 242 | 3.80 | 129.3 | 30.6 | 220 | 0.279 | 0.701 |
| Example 243 | Compound 243 | 3.82 | 125.7 | 30.6 | 213 | 0.244 | 0.724 |
| Example 244 | Compound 244 | 3.86 | 129.0 | 31.4 | 216 | 0.256 | 0.714 |
| Example 245 | Compound 245 | 3.83 | 125.2 | 30.6 | 225 | 0.233 | 0.732 |
| Example 246 | Compound 246 | 3.80 | 122.8 | 30.1 | 230 | 0.231 | 0.734 |
| Example 247 | Compound 247 | 3.84 | 120.8 | 29.5 | 218 | 0.243 | 0.725 |
| Example 248 | Compound 248 | 3.91 | 128.5 | 31.2 | 212 | 0.250 | 0.719 |
| Example 249 | Compound 249 | 3.77 | 125.2 | 30.6 | 252 | 0.233 | 0.732 |
| Example 250 | Compound 250 | 3.76 | 122.8 | 30.1 | 250 | 0.231 | 0.734 |
| Example 251 | Compound 251 | 3.78 | 126.7 | 30.5 | 255 | 0.250 | 0.722 |
| Example 252 | Compound 252 | 3.77 | 126.2 | 30.5 | 290 | 0.248 | 0.722 |
| Comparative | [Compound A] | 4.05 | 108.8 | 24.9 | 175 | 0.220 | 0.727 |
| Example 1 | | | | | | | |
| Comparative | [Compound B] | 4.01 | 108.7 | 24.1 | 165 | 0.231 | 0.719 |
| Example 2 | | | | | | | |
| Comparative | [Compound C] | 4.15 | 102.5 | 23.6 | 185 | 0.222 | 0.722 |
| Example 3 | | | | | | | |

### Example 253: Manufacturing of Organic Electroluminescent Device (Blue)

A positive electrode was formed with ITO on a substrate on which a reflective layer was formed and surface-treated with N₂ plasma or UV-ozone. HAT-CN was deposited above the positive electrode in a thickness of 10 nm as a hole injection layer (HIL). Subsequently, a hole transport layer (HTL) was formed by depositing N4,N4,N4′,N4′-tetra([1,1′-biphenyl]-4-yl)-[1,1′-biphenyl]-4,4′-diamine in a thickness of 110 nm.

A hole transport auxiliary layer was formed above the hole transport layer by forming Compound 1 by vacuum deposition in a thickness of 15 nm, and N1,N1,N6,N6-tetrakis(4-(1-silyl)phenyl)pyrene-1,6-diamine as a dopant was doped at about 3 wt % while 9,10-bis(2-naphthyl)anthracene (ADN) capable of forming a blue EML as a light emitting layer (EML) was deposited above the hole transport auxiliary layer in a thickness of 25 nm.

An electron transport layer (ETL) was deposited above the light emitting layer (EML) in a thickness of 30 nm by mixing anthracene derivative and Liq at a mass ratio of 1:1, and Liq as an electron injection layer (EIL) was deposited above the electron transport layer (ETL) in a thickness of 1 nm. Then, a mixture mixing magnesium and silver (Ag) at 9:1 was deposited in a thickness of 15 nm as a negative electrode, and N4,N4′-bis[4-[bis(3-methylphenyl)amino]phenyl]-N4,N4′-diphenyl-[1,1′-biphenyl]-4,4′-diamine (DNTPD) as a capping layer was deposited above the negative electrode in a thickness of 60 nm. An organic electroluminescent device was manufactured by bonding a sealcap containing a moisture absorbent to the capping layer using a UV curable adhesive to protect the organic electroluminescent device from 02 or moisture in the atmosphere.

### Examples 254 to 511

An organic electroluminescent device was manufactured in the same manner as Example 253 except that Compounds 2 to 259 listed in Table 22 below were used instead of Compound 1 as the hole transport auxiliary layer in Example 253.

### Comparative Examples 4 to 6

An organic electroluminescent device was manufactured in the same manner as Example 253 except that Compounds A to C were used instead of Compound 1 as the hole transport auxiliary layer in Example 253.

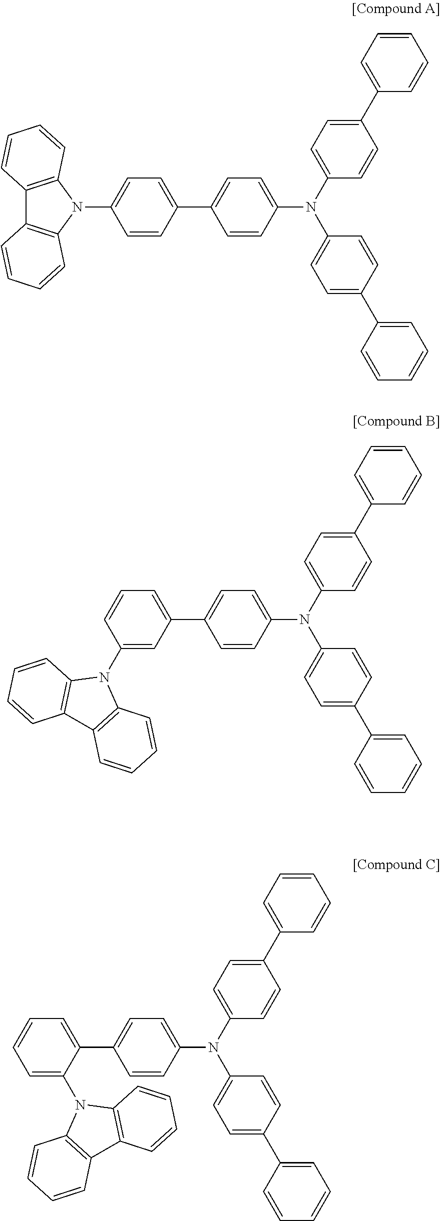 [SMILES: c1ccc(-c2ccc(N(c3ccc(-c4ccccc4)cc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4cccc(-n5c6ccccc6c6ccccc65)c4)cc3)cc2)cc1 ; c1ccc(-c2ccc(N(c3ccc(-c4ccccc4)cc3)c3ccc(-c4ccccc4-n4c5ccccc5c5ccccc54)cc3)cc2)cc1]

### Experimental Example 2: Device Performance Analysis

For the organic electroluminescent devices manufactured in Examples 253 to 511 and Comparative Examples 4 to 6, electroluminescent characteristics when driven at a current of 10 mA/cm² and lifetime reduced by 95% when driven at a constant current of 20 mA/cm² were measured, and a result of measurement is expressed in Table 22.

TABLE 22

| | Hole transport | Driving | Efficiency | | Lifetime | Color | |
|---|---|---|---|---|---|---|---|
| Items | auxiliary layer | voltage (V) | Cd/A | EQE(%) | T95(hrs) | CIEx | CIEy |
| Example 253 | Compound 1 | 3.71 | 10.2 | 18.5 | 345 | 0.122 | 0.054 |
| Example 254 | Compound 2 | 3.72 | 8.9 | 17.9 | 335 | 0.126 | 0.046 |
| Example 255 | Compound 3 | 3.62 | 8.5 | 17.2 | 345 | 0.127 | 0.045 |
| Example 256 | Compound 4 | 3.68 | 8.6 | 17.3 | 375 | 0.127 | 0.045 |
| Example 257 | Compound 5 | 3.63 | 8.4 | 17.8 | 335 | 0.128 | 0.042 |
| Example 258 | Compound 6 | 3.69 | 8.7 | 18.0 | 315 | 0.128 | 0.043 |
| Example 259 | Compound 7 | 3.69 | 8.3 | 17.3 | 325 | 0.128 | 0.043 |
| Example 260 | Compound 8 | 3.38 | 9.6 | 19.1 | 365 | 0.126 | 0.046 |
| Example 261 | Compound 9 | 3.40 | 9.1 | 18.2 | 365 | 0.127 | 0.046 |
| Example 262 | Compound 10 | 3.38 | 9.6 | 19.3 | 365 | 0.127 | 0.046 |
| Example 263 | Compound 11 | 3.47 | 9.7 | 19.1 | 380 | 0.127 | 0.047 |
| Example 264 | Compound 12 | 3.46 | 8.4 | 17.6 | 390 | 0.128 | 0.043 |
| Example 265 | Compound 13 | 3.50 | 8.1 | 17.2 | 425 | 0.129 | 0.041 |
| Example 266 | Compound 14 | 3.46 | 8.4 | 17.4 | 405 | 0.128 | 0.043 |
| Example 267 | Compound 15 | 3.55 | 8.1 | 17.3 | 405 | 0.129 | 0.041 |
| Example 268 | Compound 16 | 3.54 | 10.1 | 18.4 | 330 | 0.132 | 0.057 |
| Example 269 | Compound 17 | 3.51 | 8.8 | 17.8 | 320 | 0.136 | 0.049 |
| Example 270 | Compound 18 | 3.58 | 8.4 | 17.1 | 330 | 0.137 | 0.048 |
| Example 271 | Compound 19 | 3.64 | 8.5 | 17.2 | 360 | 0.137 | 0.048 |
| Example 272 | Compound 20 | 3.47 | 8.3 | 17.7 | 320 | 0.138 | 0.045 |
| Example 273 | Compound 21 | 3.48 | 8.6 | 17.9 | 300 | 0.138 | 0.046 |
| Example 274 | Compound 22 | 3.58 | 8.2 | 17.2 | 310 | 0.138 | 0.046 |
| Example 275 | Compound 23 | 3.68 | 9.5 | 19.0 | 350 | 0.136 | 0.049 |
| Example 276 | Compound 24 | 3.54 | 9.0 | 18.1 | 350 | 0.137 | 0.049 |
| Example 277 | Compound 25 | 3.45 | 9.5 | 19.2 | 350 | 0.137 | 0.049 |
| Example 278 | Compound 26 | 3.51 | 9.6 | 19.0 | 365 | 0.137 | 0.050 |
| Example 279 | Compound 27 | 3.54 | 8.3 | 17.5 | 375 | 0.138 | 0.046 |
| Example 280 | Compound 28 | 3.49 | 8.0 | 17.1 | 410 | 0.139 | 0.044 |
| Example 281 | Compound 29 | 3.52 | 8.3 | 17.3 | 390 | 0.138 | 0.046 |
| Example 282 | Compound 30 | 3.56 | 8.0 | 17.2 | 390 | 0.139 | 0.044 |
| Example 283 | Compound 31 | 3.55 | 8.2 | 17.4 | 310 | 0.138 | 0.046 |
| Example 284 | Compound 32 | 3.58 | 8.1 | 17.4 | 320 | 0.139 | 0.044 |
| Example 285 | Compound 33 | 3.44 | 8.0 | 17.0 | 420 | 0.140 | 0.043 |
| Example 286 | Compound 34 | 3.54 | 9.0 | 18.1 | 350 | 0.137 | 0.049 |
| Example 287 | Compound 35 | 3.55 | 8.4 | 17.6 | 360 | 0.138 | 0.046 |
| Example 288 | Compound 36 | 3.45 | 8.0 | 17.7 | 300 | 0.140 | 0.042 |
| Example 289 | Compound 37 | 3.48 | 8.1 | 17.4 | 350 | 0.139 | 0.044 |
| Example 290 | Compound 38 | 3.56 | 9.1 | 18.6 | 350 | 0.137 | 0.048 |
| Example 291 | Compound 39 | 3.57 | 10.2 | 18.6 | 295 | 0.132 | 0.057 |
| Example 292 | Compound 40 | 3.53 | 9.5 | 18.3 | 340 | 0.134 | 0.053 |
| Example 293 | Compound 41 | 3.59 | 8.5 | 17.6 | 350 | 0.138 | 0.047 |
| Example 294 | Compound 42 | 3.49 | 9.1 | 18.3 | 370 | 0.136 | 0.049 |
| Example 295 | Compound 43 | 3.59 | 9.3 | 18.8 | 380 | 0.136 | 0.049 |
| Example 296 | Compound 44 | 3.61 | 7.7 | 16.9 | 300 | 0.139 | 0.047 |
| Example 297 | Compound 45 | 3.56 | 7.6 | 16.9 | 310 | 0.140 | 0.045 |
| Example 298 | Compound 46 | 3.55 | 7.5 | 16.5 | 410 | 0.141 | 0.044 |
| Example 299 | Compound 47 | 3.60 | 8.5 | 17.6 | 340 | 0.138 | 0.050 |
| Example 300 | Compound 48 | 3.63 | 7.9 | 17.1 | 350 | 0.139 | 0.047 |
| Example 301 | Compound 49 | 3.64 | 7.5 | 17.2 | 290 | 0.141 | 0.043 |
| Example 302 | Compound 50 | 3.67 | 7.6 | 16.9 | 340 | 0.140 | 0.045 |
| Example 303 | Compound 51 | 3.53 | 8.6 | 18.1 | 340 | 0.138 | 0.049 |
| Example 304 | Compound 52 | 3.46 | 9.7 | 18.1 | 285 | 0.133 | 0.058 |
| Example 305 | Compound 53 | 3.45 | 9.0 | 17.8 | 330 | 0.135 | 0.054 |
| Example 306 | Compound 54 | 3.37 | 8.0 | 17.1 | 340 | 0.139 | 0.048 |
| Example 307 | Compound 55 | 3.35 | 8.6 | 17.8 | 360 | 0.137 | 0.050 |
| Example 308 | Compound 56 | 3.58 | 8.8 | 18.3 | 370 | 0.137 | 0.050 |
| Example 309 | Compound 57 | 3.63 | 8.5 | 18.7 | 300 | 0.140 | 0.043 |
| Example 310 | Compound 58 | 3.59 | 8.1 | 17.9 | 309 | 0.141 | 0.041 |
| Example 311 | Compound 59 | 3.67 | 8.0 | 17.4 | 290 | 0.143 | 0.038 |
| Example 312 | Compound 60 | 3.57 | 8.5 | 17.8 | 315 | 0.138 | 0.046 |
| Example 313 | Compound 61 | 3.68 | 8.1 | 18.7 | 295 | 0.141 | 0.040 |
| Example 314 | Compound 62 | 3.52 | 8.3 | 17.2 | 300 | 0.137 | 0.048 |
| Example 315 | Compound 63 | 3.50 | 10.0 | 19.1 | 330 | 0.135 | 0.053 |
| Example 316 | Compound 64 | 3.49 | 8.8 | 17.9 | 300 | 0.137 | 0.048 |
| Example 317 | Compound 65 | 3.59 | 8.6 | 17.9 | 315 | 0.137 | 0.047 |
| Example 318 | Compound 66 | 3.46 | 9.4 | 18.1 | 380 | 0.135 | 0.052 |
| Example 319 | Compound 67 | 3.48 | 8.8 | 18.3 | 360 | 0.138 | 0.047 |
| Example 320 | Compound 68 | 3.56 | 8.5 | 18.5 | 340 | 0.140 | 0.043 |
| Example 321 | Compound 69 | 3.46 | 9.0 | 18.3 | 430 | 0.137 | 0.048 |
| Example 322 | Compound 70 | 3.49 | 9.1 | 18.4 | 420 | 0.136 | 0.049 |
| Example 323 | Compound 71 | 3.51 | 8.5 | 17.5 | 380 | 0.137 | 0.048 |
| Example 324 | Compound 72 | 3.53 | 8.0 | 18.2 | 280 | 0.141 | 0.044 |
| Example 325 | Compound 73 | 3.56 | 7.6 | 17.4 | 289 | 0.142 | 0.042 |
| Example 326 | Compound 74 | 3.58 | 7.5 | 16.9 | 270 | 0.144 | 0.039 |
| Example 327 | Compound 75 | 3.60 | 8.0 | 17.3 | 295 | 0.139 | 0.047 |
| Example 328 | Compound 76 | 3.64 | 7.6 | 18.2 | 275 | 0.142 | 0.041 |
| Example 329 | Compound 77 | 3.61 | 7.8 | 16.7 | 280 | 0.138 | 0.049 |
| Example 330 | Compound 78 | 3.58 | 9.5 | 18.6 | 310 | 0.136 | 0.054 |
| Example 331 | Compound 79 | 3.62 | 8.3 | 17.4 | 280 | 0.138 | 0.049 |
| Example 332 | Compound 80 | 3.69 | 8.1 | 17.4 | 295 | 0.138 | 0.048 |
| Example 333 | Compound 81 | 3.55 | 8.9 | 17.6 | 360 | 0.136 | 0.053 |
| Example 334 | Compound 82 | 3.54 | 8.3 | 17.8 | 340 | 0.139 | 0.048 |
| Example 335 | Compound 83 | 3.56 | 8.0 | 18.0 | 320 | 0.141 | 0.044 |
| Example 336 | Compound 84 | 3.55 | 8.5 | 17.8 | 410 | 0.138 | 0.049 |
| Example 337 | Compound 85 | 3.60 | 8.6 | 17.9 | 400 | 0.137 | 0.050 |
| Example 338 | Compound 86 | 3.63 | 8.0 | 17.0 | 360 | 0.138 | 0.049 |
| Example 339 | Compound 87 | 3.53 | 9.5 | 18.4 | 295 | 0.134 | 0.052 |
| Example 340 | Compound 88 | 3.56 | 10.1 | 18.4 | 340 | 0.132 | 0.057 |
| Example 341 | Compound 89 | 3.55 | 8.5 | 17.6 | 340 | 0.138 | 0.046 |
| Example 342 | Compound 90 | 3.54 | 8.2 | 17.1 | 380 | 0.138 | 0.046 |
| Example 343 | Compound 91 | 3.55 | 8.1 | 17.0 | 380 | 0.138 | 0.045 |
| Example 344 | Compound 92 | 3.57 | 9.7 | 17.9 | 330 | 0.134 | 0.055 |
| Example 345 | Compound 93 | 3.58 | 9.6 | 18.1 | 450 | 0.135 | 0.053 |
| Example 346 | Compound 94 | 3.54 | 8.4 | 17.8 | 320 | 0.138 | 0.045 |
| Example 347 | Compound 95 | 3.56 | 8.0 | 17.9 | 320 | 0.141 | 0.040 |
| Example 348 | Compound 96 | 3.59 | 8.3 | 17.0 | 390 | 0.139 | 0.043 |
| Example 349 | Compound 97 | 3.54 | 8.2 | 17.2 | 380 | 0.139 | 0.043 |
| Example 350 | Compound 98 | 3.53 | 8.0 | 17.1 | 450 | 0.138 | 0.045 |
| Example 351 | Compound 99 | 3.59 | 8.7 | 17.8 | 410 | 0.137 | 0.048 |
| Example 352 | Compound 100 | 3.54 | 9.0 | 17.9 | 255 | 0.135 | 0.053 |
| Example 353 | Compound 101 | 3.57 | 9.6 | 17.9 | 300 | 0.133 | 0.058 |
| Example 354 | Compound 102 | 3.57 | 8.0 | 17.1 | 300 | 0.139 | 0.047 |
| Example 355 | Compound 103 | 3.62 | 7.7 | 16.6 | 340 | 0.139 | 0.047 |
| Example 356 | Compound 104 | 3.59 | 7.6 | 16.5 | 340 | 0.139 | 0.046 |
| Example 357 | Compound 105 | 3.51 | 9.2 | 17.4 | 290 | 0.135 | 0.056 |
| Example 358 | Compound 106 | 3.54 | 9.1 | 17.6 | 410 | 0.136 | 0.054 |
| Example 359 | Compound 107 | 3.50 | 7.9 | 17.3 | 280 | 0.139 | 0.046 |
| Example 360 | Compound 108 | 3.48 | 7.5 | 17.4 | 280 | 0.142 | 0.041 |
| Example 361 | Compound 109 | 3.61 | 7.8 | 16.5 | 350 | 0.140 | 0.044 |
| Example 362 | Compound 110 | 3.60 | 7.7 | 16.7 | 340 | 0.140 | 0.044 |
| Example 363 | Compound 111 | 3.65 | 7.5 | 16.6 | 410 | 0.139 | 0.046 |
| Example 364 | Compound 112 | 3.63 | 8.2 | 17.3 | 370 | 0.138 | 0.049 |
| Example 365 | Compound 113 | 3.55 | 9.1 | 18.0 | 390 | 0.136 | 0.050 |
| Example 366 | Compound 114 | 3.60 | 8.0 | 17.9 | 500 | 0.138 | 0.046 |
| Example 367 | Compound 115 | 3.59 | 10.0 | 18.5 | 380 | 0.134 | 0.055 |
| Example 368 | Compound 116 | 3.65 | 8.6 | 17.5 | 300 | 0.137 | 0.048 |
| Example 369 | Compound 117 | 3.60 | 8.7 | 18.3 | 365 | 0.138 | 0.046 |
| Example 370 | Compound 118 | 3.54 | 8.8 | 17.8 | 320 | 0.137 | 0.048 |
| Example 371 | Compound 119 | 3.67 | 9.2 | 18.3 | 300 | 0.136 | 0.049 |
| Example 372 | Compound 120 | 3.56 | 9.3 | 18.5 | 360 | 0.136 | 0.050 |
| Example 373 | Compound 121 | 3.61 | 9.1 | 18.3 | 550 | 0.136 | 0.049 |
| Example 374 | Compound 122 | 3.55 | 9.2 | 18.0 | 430 | 0.136 | 0.051 |
| Example 375 | Compound 123 | 3.55 | 8.7 | 18.7 | 430 | 0.139 | 0.044 |
| Example 376 | Compound 124 | 3.65 | 9.5 | 18.4 | 380 | 0.136 | 0.049 |
| Example 377 | Compound 125 | 3.48 | 8.8 | 18.3 | 450 | 0.138 | 0.047 |
| Example 378 | Compound 126 | 3.51 | 9.7 | 18.4 | 460 | 0.134 | 0.054 |
| Example 379 | Compound 127 | 3.51 | 9.4 | 18.4 | 510 | 0.136 | 0.049 |
| Example 380 | Compound 128 | 3.61 | 8.6 | 17.5 | 340 | 0.138 | 0.052 |
| Example 381 | Compound 129 | 3.62 | 7.5 | 17.4 | 450 | 0.140 | 0.048 |
| Example 382 | Compound 130 | 3.66 | 9.5 | 18.0 | 330 | 0.136 | 0.057 |
| Example 383 | Compound 131 | 3.67 | 8.1 | 17.0 | 250 | 0.139 | 0.050 |
| Example 384 | Compound 132 | 3.67 | 8.2 | 17.8 | 315 | 0.140 | 0.048 |
| Example 385 | Compound 133 | 3.65 | 8.3 | 17.3 | 270 | 0.139 | 0.050 |
| Example 386 | Compound 134 | 3.68 | 8.7 | 17.8 | 250 | 0.138 | 0.051 |
| Example 387 | Compound 135 | 3.62 | 8.8 | 18.0 | 310 | 0.138 | 0.052 |
| Example 387 | Compound 136 | 3.58 | 8.6 | 17.8 | 500 | 0.138 | 0.051 |
| Example 389 | Compound 137 | 3.53 | 8.7 | 17.5 | 380 | 0.138 | 0.053 |
| Example 390 | Compound 138 | 3.41 | 8.2 | 18.2 | 380 | 0.141 | 0.046 |
| Example 391 | Compound 139 | 3.59 | 9.0 | 17.9 | 330 | 0.138 | 0.051 |
| Example 392 | Compound 140 | 3.63 | 8.3 | 17.8 | 400 | 0.140 | 0.049 |
| Example 393 | Compound 141 | 3.58 | 9.2 | 17.9 | 410 | 0.136 | 0.056 |
| Example 394 | Compound 142 | 3.62 | 8.9 | 17.9 | 460 | 0.138 | 0.051 |
| Example 395 | Compound 143 | 3.59 | 9.5 | 18.4 | 355 | 0.135 | 0.049 |
| Example 396 | Compound 144 | 3.63 | 9.5 | 18.9 | 385 | 0.136 | 0.046 |
| Example 397 | Compound 145 | 3.66 | 9.5 | 18.0 | 395 | 0.134 | 0.050 |
| Example 398 | Compound 146 | 3.68 | 8.5 | 17.4 | 340 | 0.138 | 0.043 |
| Example 399 | Compound 147 | 3.65 | 9.5 | 18.0 | 370 | 0.134 | 0.050 |
| Example 400 | Compound 148 | 3.67 | 9.3 | 18.4 | 390 | 0.135 | 0.047 |
| Example 401 | Compound 149 | 3.64 | 9.3 | 18.5 | 390 | 0.136 | 0.047 |
| Example 402 | Compound 150 | 3.60 | 8.5 | 17.4 | 385 | 0.138 | 0.043 |
| Example 403 | Compound 151 | 3.59 | 8.9 | 18.3 | 360 | 0.137 | 0.045 |
| Example 404 | Compound 152 | 3.57 | 9.6 | 18.7 | 410 | 0.135 | 0.048 |
| Example 405 | Compound 153 | 3.55 | 9.6 | 18.9 | 440 | 0.135 | 0.048 |
| Example 406 | Compound 154 | 3.55 | 9.5 | 18.7 | 450 | 0.135 | 0.048 |
| Example 407 | Compound 155 | 3.54 | 9.8 | 19.0 | 475 | 0.136 | 0.046 |
| Example 408 | Compound 156 | 3.62 | 9.0 | 17.9 | 315 | 0.136 | 0.050 |
| Example 409 | Compound 157 | 3.64 | 9.0 | 18.4 | 345 | 0.137 | 0.047 |
| Example 410 | Compound 158 | 3.69 | 9.0 | 17.5 | 375 | 0.135 | 0.051 |
| Example 411 | Compound 159 | 3.67 | 8.0 | 16.9 | 300 | 0.139 | 0.044 |
| Example 412 | Compound 160 | 3.69 | 9.0 | 17.5 | 330 | 0.135 | 0.051 |
| Example 413 | Compound 161 | 3.68 | 8.8 | 17.9 | 350 | 0.136 | 0.048 |
| Example 414 | Compound 162 | 3.69 | 8.8 | 18.0 | 350 | 0.137 | 0.048 |
| Example 415 | Compound 163 | 3.67 | 8.0 | 16.9 | 380 | 0.139 | 0.044 |
| Example 416 | Compound 164 | 3.65 | 8.4 | 17.8 | 320 | 0.138 | 0.046 |
| Example 417 | Compound 165 | 3.60 | 9.1 | 18.2 | 370 | 0.136 | 0.049 |
| Example 418 | Compound 166 | 3.61 | 9.1 | 18.4 | 400 | 0.136 | 0.049 |
| Example 419 | Compound 167 | 3.59 | 9.0 | 18.2 | 410 | 0.136 | 0.049 |
| Example 420 | Compound 168 | 3.60 | 9.3 | 18.5 | 435 | 0.137 | 0.047 |
| Example 421 | Compound 169 | 3.78 | 10.0 | 18.2 | 280 | 0.125 | 0.057 |
| Example 422 | Compound 170 | 3.79 | 8.7 | 17.6 | 270 | 0.129 | 0.049 |
| Example 423 | Compound 171 | 3.69 | 8.3 | 16.9 | 280 | 0.130 | 0.048 |
| Example 424 | Compound 172 | 3.75 | 8.4 | 17.0 | 310 | 0.130 | 0.048 |
| Example 425 | Compound 173 | 3.70 | 8.2 | 17.5 | 270 | 0.131 | 0.045 |
| Example 426 | Compound 174 | 3.76 | 8.5 | 17.7 | 250 | 0.131 | 0.046 |
| Example 427 | Compound 175 | 3.76 | 8.1 | 17.0 | 260 | 0.131 | 0.046 |
| Example 428 | Compound 176 | 3.45 | 9.4 | 18.8 | 300 | 0.129 | 0.049 |
| Example 429 | Compound 177 | 3.47 | 8.9 | 17.9 | 300 | 0.130 | 0.049 |
| Example 430 | Compound 178 | 3.45 | 9.4 | 19.0 | 300 | 0.130 | 0.049 |
| Example 431 | Compound 179 | 3.54 | 9.5 | 18.8 | 315 | 0.130 | 0.050 |
| Example 432 | Compound 180 | 3.53 | 8.2 | 17.3 | 325 | 0.131 | 0.046 |
| Example 433 | Compound 181 | 3.57 | 7.9 | 16.9 | 360 | 0.132 | 0.044 |
| Example 434 | Compound 182 | 3.53 | 8.2 | 17.1 | 340 | 0.131 | 0.046 |
| Example 435 | Compound 183 | 3.62 | 7.9 | 17.0 | 340 | 0.132 | 0.044 |
| Example 436 | Compound 184 | 3.61 | 9.9 | 18.1 | 265 | 0.135 | 0.060 |
| Example 437 | Compound 185 | 3.58 | 8.6 | 17.5 | 255 | 0.139 | 0.052 |
| Example 438 | Compound 186 | 3.65 | 8.2 | 16.8 | 265 | 0.140 | 0.051 |
| Example 439 | Compound 187 | 3.71 | 8.3 | 16.9 | 295 | 0.140 | 0.051 |
| Example 440 | Compound 188 | 3.54 | 8.1 | 17.4 | 255 | 0.141 | 0.048 |
| Example 441 | Compound 189 | 3.55 | 8.4 | 17.6 | 240 | 0.141 | 0.049 |
| Example 442 | Compound 190 | 3.65 | 8.0 | 16.9 | 245 | 0.141 | 0.049 |
| Example 443 | Compound 191 | 3.75 | 9.3 | 18.7 | 285 | 0.139 | 0.052 |
| Example 444 | Compound 192 | 3.61 | 8.8 | 17.8 | 285 | 0.140 | 0.052 |
| Example 445 | Compound 193 | 3.52 | 9.3 | 18.9 | 285 | 0.140 | 0.052 |
| Example 446 | Compound 194 | 3.58 | 9.4 | 18.7 | 300 | 0.140 | 0.053 |
| Example 447 | Compound 195 | 3.61 | 8.1 | 17.2 | 310 | 0.141 | 0.049 |
| Example 448 | Compound 196 | 3.56 | 7.8 | 16.8 | 345 | 0.142 | 0.047 |
| Example 449 | Compound 197 | 3.59 | 8.1 | 17.0 | 325 | 0.141 | 0.049 |
| Example 450 | Compound 198 | 3.63 | 7.8 | 16.9 | 325 | 0.142 | 0.047 |
| Example 451 | Compound 199 | 3.62 | 8.0 | 17.1 | 245 | 0.141 | 0.049 |
| Example 452 | Compound 200 | 3.65 | 7.9 | 17.1 | 255 | 0.142 | 0.047 |
| Example 453 | Compound 201 | 3.51 | 7.8 | 16.7 | 355 | 0.143 | 0.046 |
| Example 454 | Compound 202 | 3.61 | 8.8 | 17.8 | 285 | 0.140 | 0.052 |
| Example 455 | Compound 203 | 3.62 | 8.2 | 17.3 | 295 | 0.141 | 0.049 |
| Example 456 | Compound 204 | 3.52 | 7.8 | 17.4 | 245 | 0.143 | 0.045 |
| Example 457 | Compound 205 | 3.55 | 7.9 | 17.1 | 285 | 0.142 | 0.047 |
| Example 458 | Compound 206 | 3.63 | 8.9 | 18.3 | 285 | 0.140 | 0.051 |
| Example 459 | Compound 207 | 3.64 | 10.0 | 18.3 | 247 | 0.135 | 0.060 |
| Example 460 | Compound 208 | 3.60 | 9.3 | 18.0 | 275 | 0.137 | 0.056 |
| Example 461 | Compound 209 | 3.66 | 8.3 | 17.3 | 285 | 0.141 | 0.050 |
| Example 462 | Compound 210 | 3.56 | 8.9 | 18.0 | 305 | 0.139 | 0.052 |
| Example 463 | Compound 211 | 3.66 | 9.1 | 18.5 | 315 | 0.139 | 0.052 |
| Example 464 | Compound 212 | 3.68 | 7.5 | 16.6 | 285 | 0.142 | 0.050 |
| Example 465 | Compound 213 | 3.63 | 7.4 | 16.6 | 298 | 0.143 | 0.048 |
| Example 466 | Compound 214 | 3.62 | 7.3 | 16.2 | 345 | 0.144 | 0.047 |
| Example 467 | Compound 215 | 3.67 | 8.3 | 17.3 | 275 | 0.141 | 0.053 |
| Example 468 | Compound 216 | 3.70 | 7.7 | 16.8 | 285 | 0.142 | 0.050 |
| Example 469 | Compound 217 | 3.71 | 7.3 | 16.9 | 256 | 0.144 | 0.046 |
| Example 470 | Compound 218 | 3.74 | 7.4 | 16.6 | 275 | 0.143 | 0.048 |
| Example 471 | Compound 219 | 3.60 | 8.4 | 17.8 | 275 | 0.141 | 0.052 |
| Example 472 | Compound 220 | 3.53 | 9.5 | 17.8 | 257 | 0.136 | 0.061 |
| Example 473 | Compound 221 | 3.52 | 8.8 | 17.5 | 252 | 0.138 | 0.057 |
| Example 474 | Compound 222 | 3.44 | 7.8 | 16.8 | 261 | 0.142 | 0.051 |
| Example 475 | Compound 223 | 3.42 | 8.4 | 17.5 | 295 | 0.140 | 0.053 |
| Example 476 | Compound 224 | 3.65 | 8.6 | 18.0 | 305 | 0.140 | 0.053 |
| Example 477 | Compound 225 | 3.70 | 8.3 | 18.4 | 257 | 0.143 | 0.046 |
| Example 478 | Compound 226 | 3.66 | 7.9 | 17.6 | 244 | 0.144 | 0.044 |
| Example 479 | Compound 227 | 3.74 | 7.8 | 17.1 | 267 | 0.146 | 0.041 |
| Example 480 | Compound 228 | 3.64 | 8.3 | 17.5 | 250 | 0.141 | 0.049 |
| Example 481 | Compound 229 | 3.75 | 7.9 | 18.4 | 277 | 0.144 | 0.043 |
| Example 482 | Compound 230 | 3.59 | 8.1 | 16.9 | 255 | 0.140 | 0.051 |
| Example 483 | Compound 231 | 3.57 | 9.8 | 18.8 | 265 | 0.138 | 0.056 |
| Example 484 | Compound 232 | 3.56 | 8.6 | 17.6 | 245 | 0.140 | 0.051 |
| Example 485 | Compound 233 | 3.66 | 8.4 | 17.6 | 250 | 0.140 | 0.050 |
| Example 486 | Compound 234 | 3.53 | 9.2 | 17.8 | 268 | 0.138 | 0.055 |
| Example 487 | Compound 235 | 3.55 | 8.6 | 18.0 | 259 | 0.141 | 0.050 |
| Example 488 | Compound 236 | 3.63 | 8.3 | 18.2 | 275 | 0.143 | 0.046 |
| Example 489 | Compound 237 | 3.53 | 8.8 | 18.0 | 365 | 0.140 | 0.051 |
| Example 490 | Compound 238 | 3.56 | 8.9 | 18.1 | 355 | 0.139 | 0.052 |
| Example 491 | Compound 239 | 3.58 | 8.3 | 17.2 | 315 | 0.140 | 0.051 |
| Example 492 | Compound 240 | 3.60 | 7.8 | 17.9 | 270 | 0.144 | 0.047 |
| Example 493 | Compound 241 | 3.63 | 7.4 | 17.1 | 265 | 0.145 | 0.045 |
| Example 494 | Compound 242 | 3.65 | 7.3 | 16.6 | 249 | 0.147 | 0.042 |
| Example 495 | Compound 243 | 3.67 | 7.8 | 17.0 | 254 | 0.142 | 0.050 |
| Example 496 | Compound 244 | 3.71 | 7.4 | 17.9 | 272 | 0.145 | 0.044 |
| Example 497 | Compound 245 | 3.68 | 7.6 | 16.4 | 263 | 0.141 | 0.052 |
| Example 498 | Compound 246 | 3.65 | 9.3 | 18.3 | 245 | 0.139 | 0.057 |
| Example 499 | Compound 247 | 3.69 | 8.1 | 17.1 | 252 | 0.141 | 0.052 |
| Example 500 | Compound 248 | 3.76 | 7.9 | 17.1 | 256 | 0.141 | 0.051 |
| Example 501 | Compound 249 | 3.62 | 8.7 | 17.3 | 295 | 0.139 | 0.056 |
| Example 502 | Compound 250 | 3.61 | 8.1 | 17.5 | 275 | 0.142 | 0.051 |
| Example 503 | Compound 251 | 3.63 | 7.8 | 17.7 | 255 | 0.144 | 0.047 |
| Example 504 | Compound 252 | 3.62 | 8.3 | 17.5 | 345 | 0.141 | 0.052 |
| Example 505 | Compound 253 | 3.58 | 8.4 | 17.8 | 310 | 0.137 | 0.048 |
| Example 506 | Compound 254 | 3.69 | 8.7 | 17.6 | 325 | 0.130 | 0.043 |
| Example 507 | Compound 255 | 3.65 | 8.2 | 16.8 | 265 | 0.139 | 0.052 |
| Example 508 | Compound 256 | 3.51 | 7.8 | 16.7 | 330 | 0.142 | 0.047 |
| Example 509 | Compound 257 | 3.62 | 7.7 | 16.8 | 285 | 0.142 | 0.050 |
| Example 510 | Compound 258 | 3.63 | 7.9 | 17.1 | 330 | 0.139 | 0.047 |
| Example 511 | Compound 259 | 3.57 | 9.8 | 18.8 | 265 | 0.138 | 0.056 |
| Comparative | [Compound A] | 3.82 | 6.9 | 15.5 | 210 | 0.140 | 0.042 |
| Example 4 | | | | | | | |
| Comparative | [Compound B] | 3.80 | 7.1 | 15.9 | 225 | 0.140 | 0.043 |
| Example 5 | | | | | | | |
| Comparative | [Compound C] | 3.83 | 7.0 | 15.8 | 205 | 0.139 | 0.044 |
| Example 6 | | | | | | | |

Compounds 253 to 259 expressed in Table 22 are as follows.

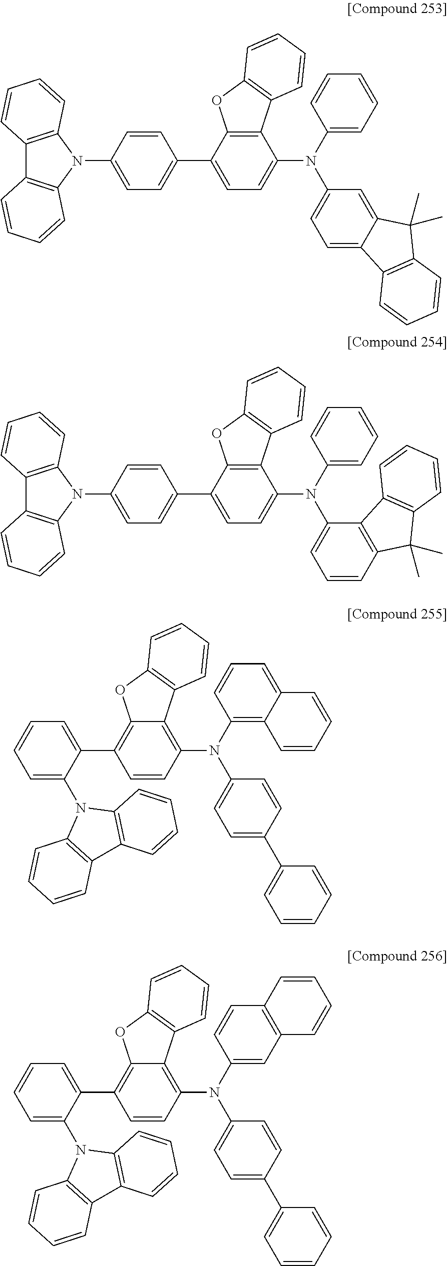 [SMILES: CC1(C)c2ccccc2-c2ccc(N(c3ccccc3)c3ccc(-c4ccc(-n5c6ccccc6c6ccccc65)cc4)c4oc5ccccc5c34)cc21 ; CC1(C)c2ccccc2-c2c(N(c3ccccc3)c3ccc(-c4ccc(-n5c6ccccc6c6ccccc65)cc4)c4oc5ccccc5c34)cccc21 ; c1ccc(-c2ccc(N(c3cccc4ccccc34)c3ccc(-c4ccccc4-n4c5ccccc5c5ccccc54)c4oc5ccccc5c34)cc2)cc1 ; c1ccc(-c2ccc(N(c3ccc4ccccc4c3)c3ccc(-c4ccccc4-n4c5ccccc5c5ccccc54)c4oc5ccccc5c34)cc2)cc1]

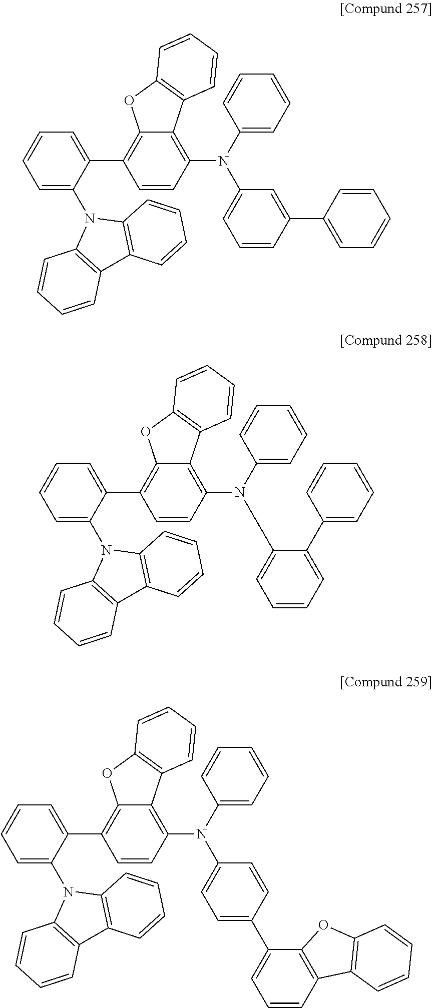 [SMILES: c1ccc(-c2cccc(N(c3ccccc3)c3ccc(-c4ccccc4-n4c5ccccc5c5ccccc54)c4oc5ccccc5c34)c2)cc1 ; c1ccc(-c2ccccc2N(c2ccccc2)c2ccc(-c3ccccc3-n3c4ccccc4c4ccccc43)c3oc4ccccc4c23)cc1 ; c1ccc(N(c2ccc(-c3cccc4c3oc3ccccc34)cc2)c2ccc(-c3ccccc3-n3c4ccccc4c4ccccc43)c3oc4ccccc4c23)cc1]

According to the experimental result in Tables 21 and 22, it was confirmed that when the compounds of the present invention were used as a material for the hole transport auxiliary layer of the organic electroluminescent device, the driving voltage was low, and excellent device efficiency characteristics and long lifetime characteristics were shown as compared to Comparative Examples.

In other words, it was confirmed that the compounds in Comparative Examples are compounds not bonded to dibenzofuran or dibenzothiophene between the amine group and the carbazole group substituted with the aryl group, as shown in the experimental result of Comparative Examples in Tables 21 and 22, the driving voltage was high and the device efficiency and lifetime were low.

According to the experimental results in Tables 21 and 22, it was confirmed that when the compounds in part or whole substituted with deuterium of the present invention were used as a material for the hole transport auxiliary layer of the organic electroluminescent device, the driving voltage was low, and excellent device efficiency characteristics and long lifetime characteristics were shown as compared to Comparative Examples.

The present invention is not limited by the embodiments disclosed above in the specification, and it is apparent that various modifications may be made by those skilled in the art within the scope of the technical spirit of the present invention. In addition, even when the operational effects according to the configuration of the present invention have not been explicitly described in the description of the embodiments of the present invention, it goes without saying that the effects predictable by the corresponding configuration should also be recognized.

### INDUSTRIAL APPLICABILITY

The present invention relates to an organic compound and an organic electroluminescent device including the same.

1. A compound represented by Chemical Formula 1: 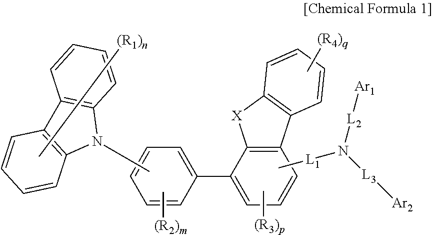
wherein,
n is an integer from 0 to 8,
m and q is the same as or different from each other and is each independently an integer from 0 to 4,
p is an integer from 0 to 2,
X is O or S,
L₁ to L₃ are the same as or different from each other and are each independently selected from the group consisting of a single bond, a substituted or unsubstituted arylene group having 6 to 30 carbon atoms, a substituted or unsubstituted heteroarylene group having 1 to 30 carbon atoms, a substituted or unsubstituted alkylen group having 1 to 10 carbon atoms, a substituted or unsubstituted cycloalkylene group having 3 to 10 carbon atoms, a substituted or unsubstituted alkenylene group with 2 to 10 carbon atoms, a substituted or unsubstituted cycloalkenylene group having 3 to 10 carbon atoms, a substituted or unsubstituted heteroalkylene group having 1 to 10 carbon atoms, a substituted or unsubstituted heterocycloalkylene group having 2 to 10 carbon atoms, a substituted or unsubstituted heteroalkenylene group having 2 to 10 carbon atoms, and a substituted or unsubstituted heterocycloalkenylene group having 2 to 10 carbon atoms,
Ar₁ and Ar₂ are the same as or different from each other and are each independently selected from the group consisting of a substituted or unsubstituted aryl group having 6 to 30 carbon atoms, a substituted or unsubstituted heteroaryl group having 1 to 30 carbon atoms, a substituted or unsubstituted alkyl group having 1 to 20 carbon atoms, a substituted or unsubstituted cycloalkyl group having 3 to 20 carbon atoms, a substituted or unsubstituted heteroalkyl group having 1 to 20 carbon atoms, a substituted or unsubstituted heterocycloalkyl group having 2 to 20 carbon atoms, a substituted or unsubstituted aralkyl group having 6 to 30 carbon atoms, a substituted or unsubstituted alkenyl group having 2 to 20 carbon atoms, a substituted or unsubstituted cycloalkenyl group having 3 to 20 carbon atoms, and a substituted or unsubstituted heteroalkenyl groups having 2 to 20 carbon atoms, and
R₁ to R₄ are the same as or different from each other and are each independently selected from the group consisting of hydrogen, deuterium, a cyano group, a nitro group, a halogen group, a hydroxy group, a substituted or unsubstituted alkylthio group having 1 to 4 carbon atoms, a substituted or unsubstituted alkyl group having 1 to 30 carbon atoms, a substituted or unsubstituted cycloalkyl group having 3 to 20 carbon atoms, a substituted or unsubstituted alkenyl group having 2 to 30 carbon atoms, a substituted or unsubstituted alkynyl group having 2 to 24 carbon atoms, a substituted or unsubstituted aralkyl group having 7 to 30 carbon atoms, a substituted or unsubstituted aryl group having 6 to 30 carbon atoms, a substituted or unsubstituted heteroaryl group having 1 to 60 carbon atoms, a substituted or unsubstituted heteroarylalkyl group having 2 to 30 carbon atoms, a substituted or unsubstituted alkoxy group having 1 to 30 carbon atoms, a substituted or unsubstituted alkylamino group having 1 to 30 carbon atoms, a substituted or unsubstituted arylamino group having 6 to 30 carbon atoms, a substituted or unsubstituted aralkylamino group having 7 to 30 carbon atoms, a substituted or unsubstituted heteroarylamino group having 1 to 24 carbon atoms, a substituted or unsubstituted alkylsilyl group having 1 to 30 carbon atoms, a substituted or unsubstituted arylsilyl group having 6 to 30 carbon atoms, and a substituted or unsubstituted aryloxy group having 6 to 30 carbon atoms.

2. The compound of claim 1, wherein the compound represented by Chemical Formula 1 is represented by Chemical Formulas 2 to 4 below: 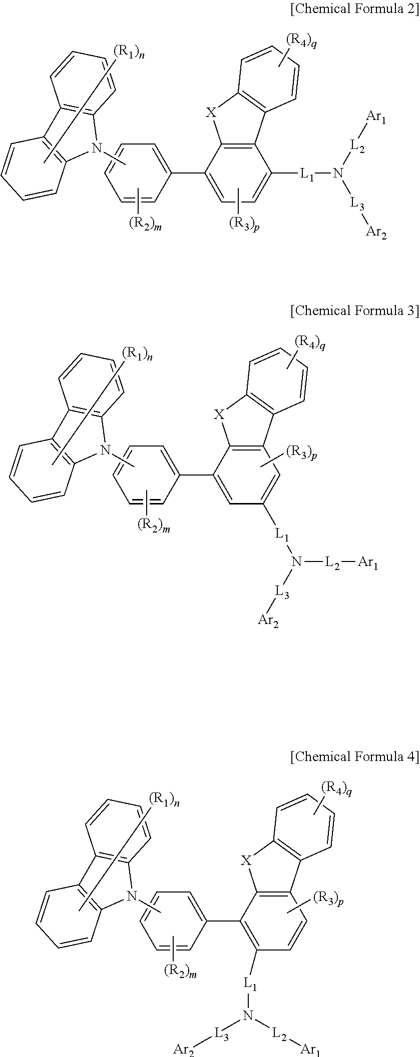
wherein,
n, m, p, q, X, L₁ to L₃, Ar₁, Ar₂, and R₁ to R₄ are same as defined in claim 1.

3. The compound of claim 2, wherein the compound represented by Chemical Formula 2 is represented by Chemical Formulas 5 to 7 below: 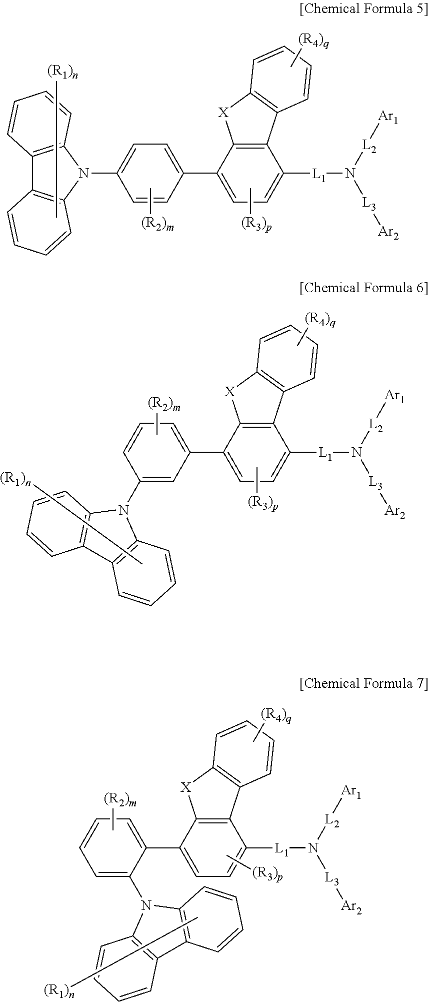
wherein,
n, m, p, q, X, L₁ to L₃, Ar₁, Ar₂, and R₁ to R₄ are same as defined in claim 1.

4. The compound of claim 2, wherein the compound represented by Chemical Formula 3 is represented by Chemical Formulas 8 to 10 below: 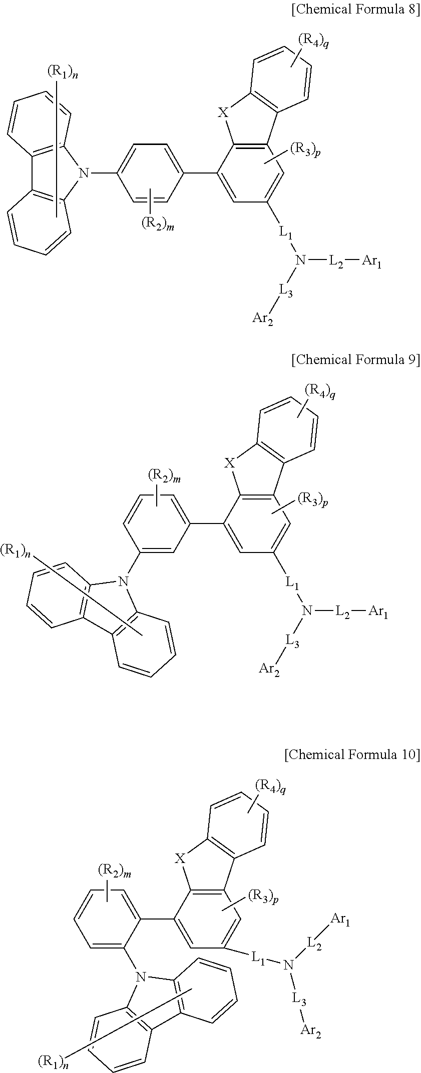
wherein,
n, m, p, q, X, L₁ to L₃, Ar₁, Ar₂, and R₁ to R₄ are same as defined in claim 1.

5. The compound of claim 2, wherein the compound represented by Chemical Formula 4 is represented by Chemical Formulas 11 to 13 below: 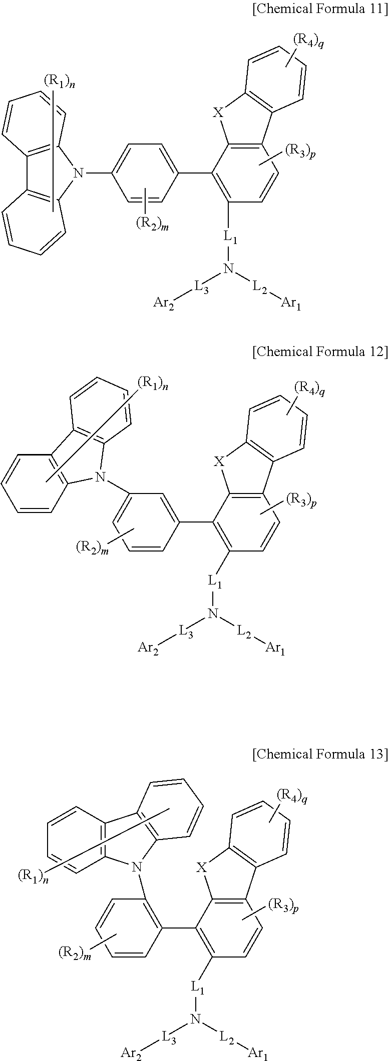
wherein,
n, m, p, q, X, L₁ to L₃, Ar₁, Ar₂, and R₁ to R₄ are same as defined in claim 1.

6. The compound of claim 1, wherein L₁ to L₃ are the same as or different from each other and are each independently be selected from the group consisting of a single bond, a substituted or unsubstituted arylene group having 6 to 30 carbon atoms, and a substituted or unsubstituted heteroarylene group having 1 to 30 carbon atoms.

7. The compound of claim 1, wherein Ar₁ and Ar₂ are the same as or different from each other and are each independently be selected from the group consisting of compounds represented by Chemical Formulas 14 to 17 below: 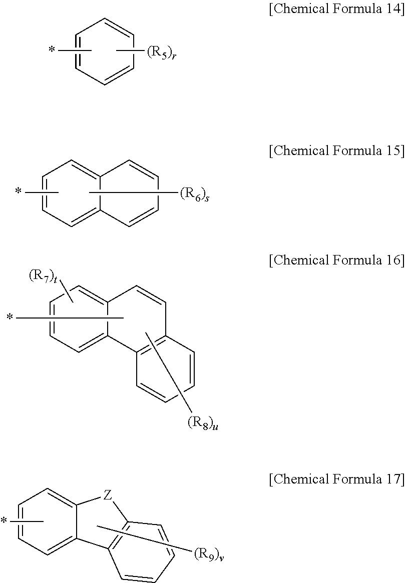
wherein,
* indicates a portion which is bonded,
r is an integer from 0 to 5,
s and v are the same as or different from each other and are each independently an integer from 0 to 7,
t is an integer from 0 to 4,
u is an integer from 0 to 6,
Z is C(R₁₀)(R₁₁), N(R₁₂), O or S, and
R₅ to R₁₂ are the same as or different from each other, are each independently selected from the group consisting of hydrogen, deuterium, a cyano group, a nitro group, a halogen group, a hydroxy group, a substituted or unsubstituted alkylthio group having 1 to 4 carbon atoms, a substituted or unsubstituted alkyl group having 1 to 30 carbon atoms, a substituted or unsubstituted cycloalkyl group having 3 to 20 carbon atoms, a substituted or unsubstituted alkenyl group having 2 to 30 carbon atoms, a substituted or unsubstituted alkynyl group having 2 to 24 carbon atoms, a substituted or unsubstituted aralkyl group having 7 to 30 carbon atoms, a substituted or unsubstituted aryl group having 6 to 30 carbon atoms, a substituted or unsubstituted heteroaryl group having 1 to 60 carbon atoms, a substituted or unsubstituted heteroarylalkyl group having 2 to 30 carbon atoms, a substituted or unsubstituted alkoxy group having 1 to 30 carbon atoms, a substituted or unsubstituted alkylamino group having 1 to 30 carbon atoms, a substituted or unsubstituted arylamino group having 6 to 30 carbon atoms, a substituted or unsubstituted aralkylamino group having 7 to 30 carbon atoms, a substituted or unsubstituted heteroarylamino group having 1 to 24 carbon atoms, a substituted or unsubstituted alkylsilyl group having 1 to 30 carbon atoms, a substituted or unsubstituted arylsilyl group having 6 to 30 carbon atoms, and a substituted or unsubstituted aryloxy group having 6 to 30 carbon atoms.

8. The compound of claim 1, wherein Ar₁ and Ar₂ are the same as or different from each other and are each independently selected from the group consisting of a substituted or unsubstituted phenylene group, a substituted or unsubstituted biphenylene group, a substituted or unsubstituted terphenylene group, a substituted or unsubstituted naphthalenyl group, a substituted or unsubstituted phenanthrenyl group, a substituted or unsubstituted fluorene group, a substituted or unsubstituted spirofluorene group, a substituted or unsubstituted dibenzofuran group, a substituted or unsubstituted dibenzothiophene group, a substituted or unsubstituted furan group, and a substituted or unsubstituted carbazole group.

9. An organic electroluminescent device comprising:
a first electrode; a second electrode provided to face the first electrode; and one or more organic layers provided between the first electrode and the second electrode,
wherein at least one of the one or more organic layers includes the compound of claim 1.

10. The organic electroluminescent device of claim 9, wherein the organic layer is selected from the group consisting of a hole injection layer, a hole transport layer, a hole transport auxiliary layer, a light emitting layer, an electron transport layer, and an electron injection layer.

11. The organic electroluminescent device of claim 9, wherein the organic layer is a hole transport auxiliary layer.
